(19) United States
(12) Patent Application Publication
(10) Pub. No.: US 2025/0282783 A1
(43) Pub. Date: Sep. 11, 2025

(54) PROTEIN STABILIZING COMPOUNDS CONTAINING USP28 AND/OR USP25 TARGETING LIGANDS

(71) Applicant: Stablix, Inc., Boston, MA (US)

(72) Inventors: Kevin Tyler Sprott, Boston, MA (US); Moses Moustakim, Boston, MA (US); Mark George Saulnier, Higganum, CT (US); Richard James Steel, Foster City, CA (US); Jorden Kass, Arlington, MA (US)

(21) Appl. No.: 18/723,820

(22) PCT Filed: Dec. 22, 2022
(86) PCT No.: PCT/US2022/053863

§ 371 Date: Jun. 24, 2024

Related U.S. Application Data
(60) Provisional application No. 63/292,950, filed on Dec. 22, 2021.

(51) Int. Cl. C07D 487/04; A61K 31/4155; A61K 31/4365; A61K 31/519; C07D 403/12

(52) U.S. Cl. CPC C07D 487/04; A61K 31/4155; A61K 31/4365; A61K 31/519; C07D 403/12

(57) ABSTRACT

This invention provides protein stabilizing compounds that have a USP28 Targeting Ligand or USP25 Targeting Ligand, a Protein Targeting Ligand, and optionally a Linker, wherein the Target Protein is ubiquitinated. The protein stabilizing compounds of the present invention can be used to deubiquitinate the Ubiquitinated Target Protein and thus increase the concentration and/or function of the Target Protein.

### CROSS-REFERENCE TO RELATED APPLICATIONS

This application claims the benefit of U.S. Provisional Application No. 63/292,950 filed Dec. 22, 2021. This application is incorporated by reference in its entirety for all purposes.

### FIELD OF THE INVENTION

This invention provides bifunctional molecules that stabilize Target Ubiquitinated Proteins, compositions, and methods of use thereof. The bifunctional molecules include a USP28 Targeting Ligand, a Ubiquitinated Protein Targeting Ligand, and optionally a Linker that connects the two for the restoration of the Target Protein to treat a disorder mediated by deficiencies of the Target Protein.

### BACKGROUND OF THE INVENTION

The ubiquitination of proteins is a dynamic multifaceted post-translational modification that allows the body to mark proteins for degradation, sub-cellular localization, and translocation.

Ubiquitin is a 76-amino acid protein that has several locations that can attach to other ubiquitins and other proteins. Ubiquitin commonly attaches to proteins at one of seven lysine residues or on the N-terminus. These reactive sites on ubiquitin can then be modified by other ubiquitin peptides or ubiquitin-like molecules (for example SUMO or NEDD8). The resulting three-dimensional polyubiquitin structure can be complex and can provide a multitude of signals. Swatek et. al., “Ubiquitin Modifications” Cell Research 2016 (26) 399. One of the common signals given by ubiquitin is that of proteasomal degradation. More than 700 E3 ubiquitin ligase proteins have been identified and these ligases can recognize ubiquitinated proteins and then orchestrate a complex cascade that results in protein degradation. Humphreys et. al., “The Role of E3 Ubiquitin Ligases in the Development and Progression of Glioblasoma” Cell Death & Differentiation 2021 (28) 522.

Difficult to treat diseases can occur when ubiquitination signals the degradation of proteins that the body needs. For example, in cystic fibrosis one or more mutations in the cystic fibrosis transmembrane conductance regulator (CFTR) gene causes CFTR to be less efficient in transporting ions in and out of the cellular membrane. Lee et. al., “Interference with Ubiquitination in CFTR Modifies Stability of Core Glycosylated and Cell Surface Pools” Mol. Cell Biol. 2014 (34) 2554. The body recognizes the mutant CFTR proteins as deficient and ubiquitinates them to signal degradation and thus makes the inability to transport ions in and out of the cell membrane even more pronounced. The result is a thickening of mucus, difficulty breathing, and eventual death.

The body has deubiquitinase proteins (DUBs) that partially or fully remove ubiquitin from proteins. There are over one hundred known DUBs. DUBS have been split into five families: the ubiquitin-specific proteases (USPs), the ovarian tumor proteases (OTUs), the ubiquitin C-terminal hydrolases (UCHs), the Josephin family, and the motif interacting with ubiquitin containing novel DUB family (MINDY). Mevissen et. al., “Mechanisms of Deubiquitinase Specificity and Regulation” Annu. Rev. Biochem. 2017 (86) 159. These DUBS have specificity for different functions and cleave different bonds in polyubiquitin.

The Colecraft lab has developed engineered DUB proteins “enDUBs” that have a highly selective nanobody portion connected to a DUB. Kanner et. al., “Targeted Deubiquitination Rescues Distinct Trafficking-Deficient Ion Channelopathies” Nature Methods 2020 (17) 1245. These molecules target a protein of interest, deubiquitinate it, and restore its function. Various enDUBs are disclosed in WO2019/090234, WO2020/198637, and WO2021/146390. Heterobifunctional molecules for targeted protein stabilization are described in WO2021/146386A1.

Locki Therapeutics Limited has described the use of small molecule compounds containing a protein targeting ligand, a linker, and a DUB targeting ligand for deubiquitinating the protein of interest in WO2020/169650. Locki Therapeutics has also disclosed USP7- and USP5-specific heterobifunctional compounds in WO2022/148821 and WO2022/148822.

The Nomura lab has described small molecule compounds containing a protein targeting ligand, a linker, and a DUB targeting ligand to deubiquitinate CFTR. Henning et. al., “Deubiquitinase-Targeting Chimeras for Targeted Protein Stabilization” bioRxiv 2021 441959 and WO2022/232643.

The Liu lab has disclosed small molecules that bind to USP28 in “Discovery of [1,2,3]triazolo[4,5-d]pyrimidine derivatives as highly potent, selective, and cellularly active USP28 inhibitors” Acta Pharmaceutica Sinica B 2020, 10(8), 1476-1491.

The Buhrlage lab has disclosed small molecules that bind to USP28 in WO 2022/035804, WO 2022/035805, and WO 2022/035806. Additional USP28 and USP25 ligands are described in “Identification and characterization of dual inhibitors of the USP25/28 deubiquitinating enzyme subfamily” ACS Chem. Biol. 12, 3113-3125 (2017); “USP28 deletion and small molecule inhibition destabilizes c-Myc and elicits regression of squamous cell lung carcinoma” bioRxiv 2021 37705; CN 111909181; CN112898314; U.S. Pat. No. 10,913,753; US 2019/359,628; WO 2017/139779; WO 2020/224652; and WO 2020/033709.

Despite these efforts their remains a need to develop small molecule protein function restoring molecules, along with their uses for therapeutic purposes and methods of manufacture.

### SUMMARY OF THE INVENTION

Protein stabilizing and/or function restoring bifunctional compounds and their uses and manufacture are provided that stabilize a Target Ubiquitinated Protein by deubiquitinating it. In some embodiments, the protein stabilizing and/or function restoring bifunctional compound restores some amount of the protein's function. The protein stabilizing and/or function restoring bifunctional compounds described herein include a Ubiquitin Specific Protease 28 (USP28) and or Ubiquitin Specific Protease 25 (USP25) Targeting Ligand, a Ubiquitinated Protein Targeting Ligand, and optionally a Linker that links the two. USP28 is a cysteine protease that can cleave major polyubiquitin bonds including for example lysine 11, lysine 48, and lysine 63. USP25 is a close homolog of USP28 and can cleave lysine 48 and lysine 63 linked polyubiquitin bonds. USP28 is a key regulator of ubiquitination in protein degradation pathways. By interacting with USP28 and/or USP25 and a Target Ubiquitinated Protein the protein stabilizing compounds described herein can restore a target protein's function and can thus be used to treat loss of function disorders.

When USP28 or USP25 removes ubiquitins from a protein, the proteasomal degradation of the protein may be prevented or minimized (i.e. the protein is stabilized). Further, the protein may resume its activity (i.e. the protein's function is restored).

A selected compound described herein removes ubiquitin from the Target Ubiquitinated Protein in a manner that stabilizes the protein and in some embodiments restores the protein's function. For example, a compound of the present invention may increase a target protein's function by at least about 1%, 5%, 10%, 15%, 20%, 25%, 30%, 40%, 50%, 60%, 70%, 80%, 90%, 100%, or more, as compared to the target protein's level of function in the absence of the compound. In certain embodiments, the protein's function may be enhanced over the protein as existing in the cell prior to treatment with the compound described herein. When the target protein has a loss of function mutation a compound of the present invention may restore its function relative to the wild type protein or relative to the mutated form.

By both stabilizing and restoring the protein's function various disorders that are caused by a deficiency of a protein's activity can be treated. For example, disorders caused by loss of function protein mutations or haploid insufficiency can be treated by restoring the function of the downregulated wildtype protein of interest or a mutant thereof. Difficult to treat cancers can also be treated with a protein stabilizing compound of the present invention. For example, cancers that downregulate tumor suppressors can be treated by restoring the function of the tumor suppressor. A protein stabilizing compound described herein can also prompt an immunological response in the treatment of cancer and thus treat the cancer by activating the immune system.

In certain aspects of the invention a protein stabilizing compound is used in combination with a protein activating compound such as an agonist, potentiator, chaperone, or corrector to treat a disease mediated by the Target Ubiquitinated Protein. This protein activating compound can either be administered separately or may be the Ubiquitinated Protein Targeting Ligand used in the heterobifunctional compound. In other aspects the protein stabilizing compound prevents degradation of the Target Ubiquitinated Protein and that protein forms one or more complexes with downstream phenotypic effects. In certain embodiments the protein stabilizing compound stabilizes and restores the proteins activity.

In certain embodiments the USP28 Targeting Ligand used in the present invention is an inhibitor of USP28. Despite being an inhibitor of USP28, a USP28 Targeting Ligand promotes the deubiquitination, stabilization, and/or restoration of activity for the Targeted Protein when used within a compound described herein. In certain embodiments the USP28 Targeting Ligand also binds to USP25. In certain embodiments the USP28 Targeting Ligand binds an allosteric site and does not cause significant inhibition of USP28. In certain embodiments the USP28 Targeting Ligand binds an allosteric site with inhibitor activity. In other embodiments the USP28 Targeting Ligand binds an active site.

In certain embodiments the USP28 Targeting Ligand used in the present invention is not an inhibitor of USP28. For example, in certain embodiments the USP28 Targeting Ligand is an agonist, activator, potentiator, or ligand without appreciable binding activity.

In certain aspects a protein stabilizing compound of Formula I is schematically shown as Formula I:

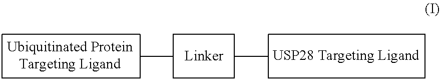

or a pharmaceutically acceptable salt thereof;
wherein:
the Ubiquitinated Protein Targeting Ligand is a ligand that binds a Target Ubiquitinated Protein; in certain embodiments the Protein's biological function can be fully or partially restored by deubiquitination as described herein;
the Linker is a bond or a bivalent moiety that links the Ubiquitinated Protein Targeting Ligand and the USP28 Targeting Ligand; and
the USP28 Targeting Ligand is a USP28 Targeting Ligand described herein for example a compound in FIG. 1 that binds USP28.

In certain embodiments the USP28 Targeting Ligand also interacts with USP25. In certain embodiments the USP28 Targeting Ligand is at least about 2-, 3-, 4-, 5-, 10-, 15-, 20-, 25-, 30-, 35-, 40-, 45-, 50-, 100-, or 500-fold selective for USP28 over other DUBs including for example USP25.

In certain embodiments the compound of the present invention is of Formula:

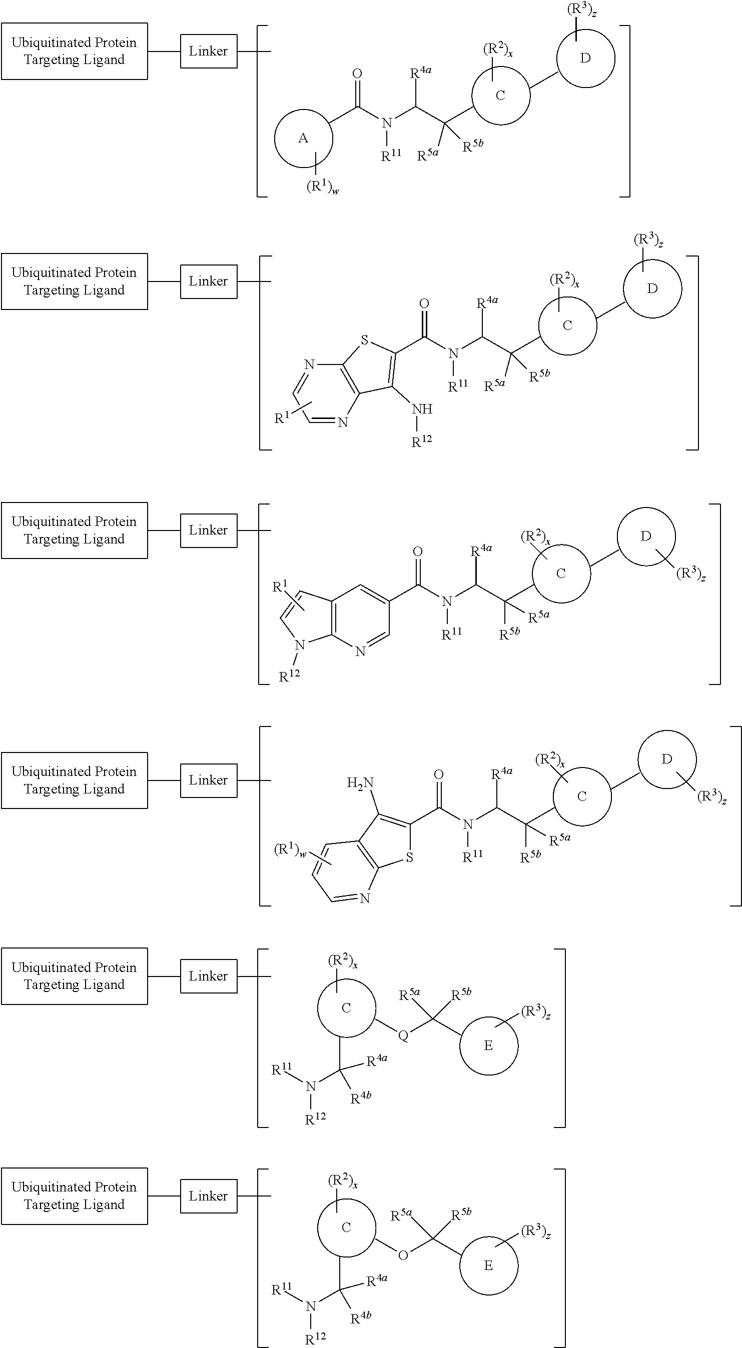

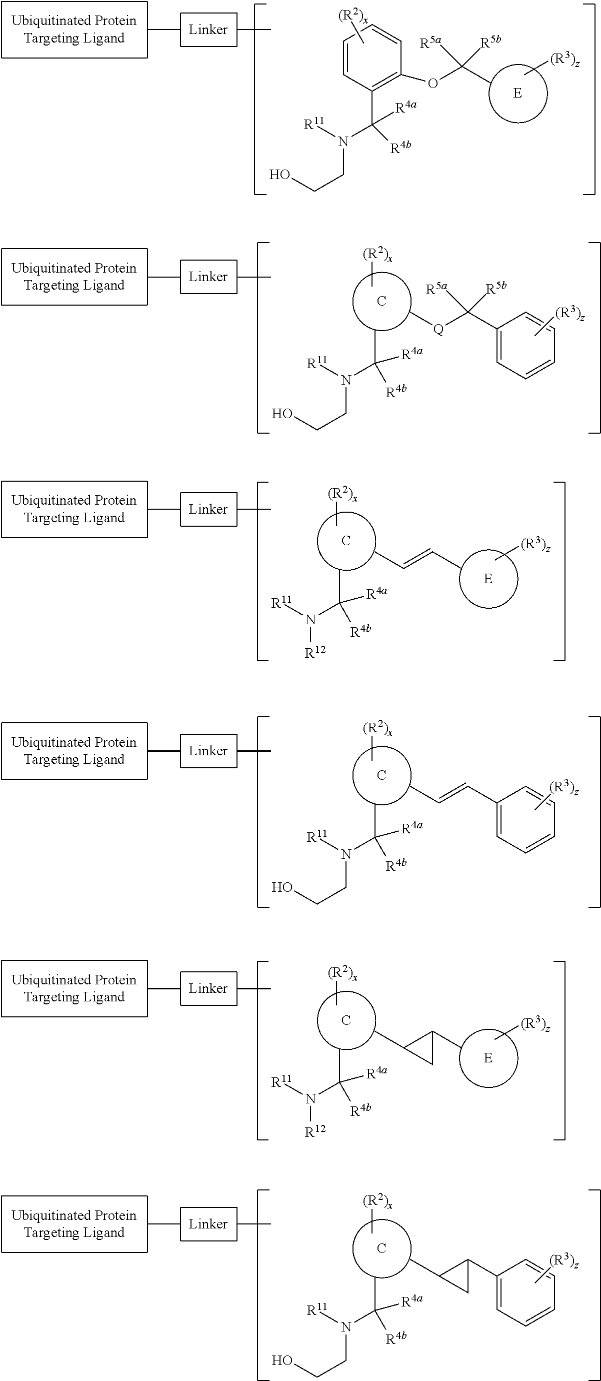

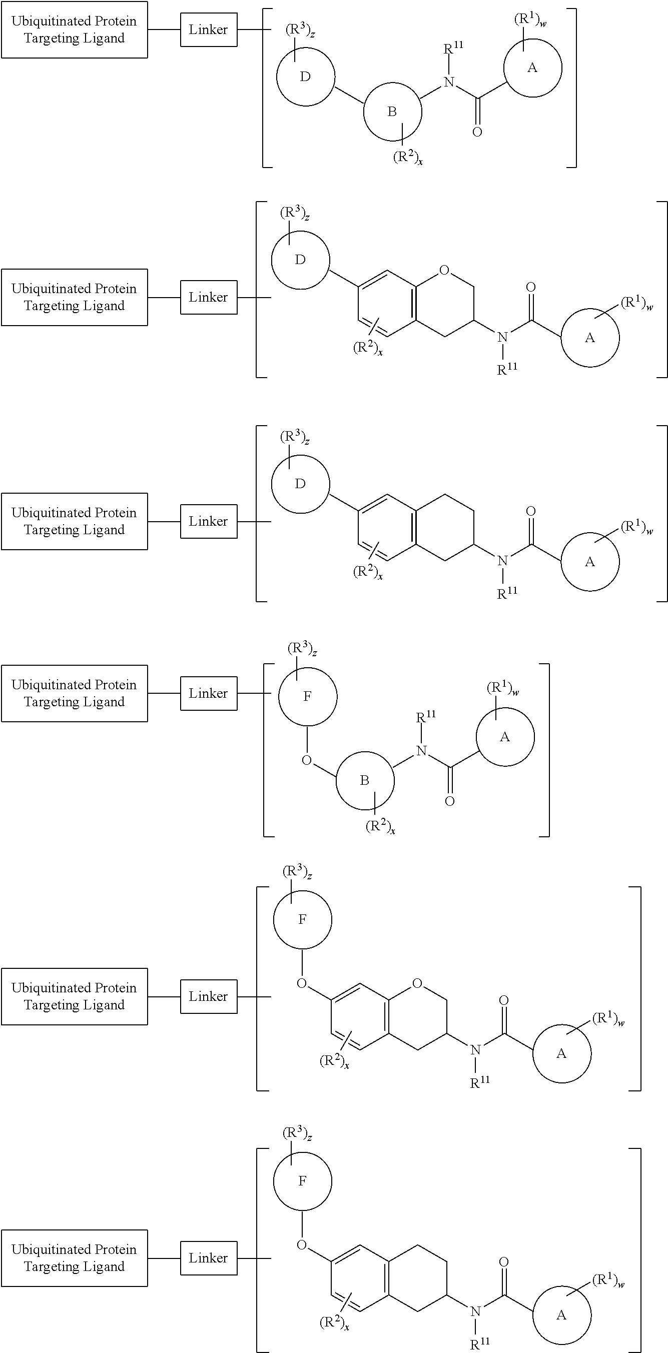

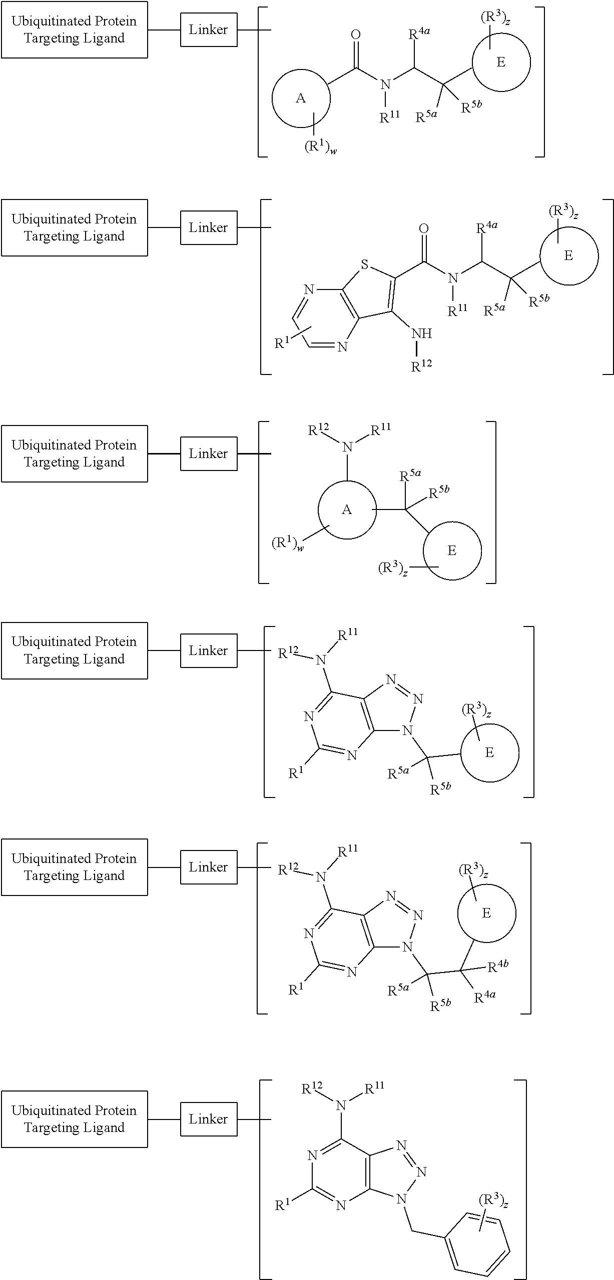

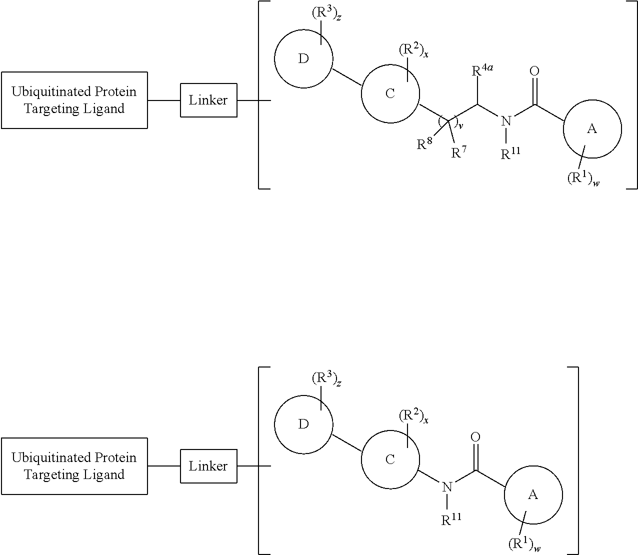

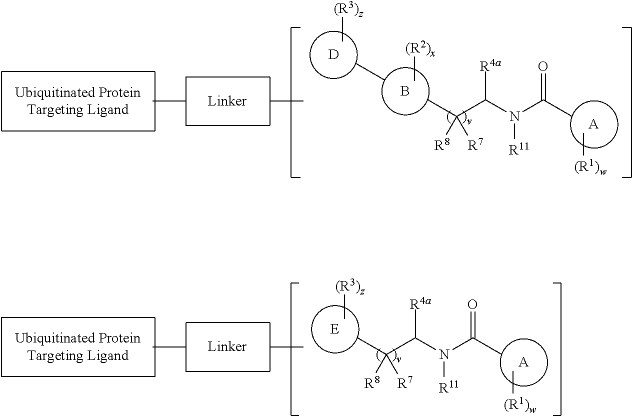

or a pharmaceutically acceptable salt thereof.
wherein:
v is 0, 1, 2, or 3;
w is 0, 1, 2, 3, or 4 as allowed by valence;
x is 0, 1, 2, 3, or 4 as allowed by valence;
z is 0, 1, 2, 3, or 4 as allowed by valence;
Q is O, NR¹¹, CR⁷R⁸, or S;
R¹ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²¹;
R² is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²²;
R³ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²³;
R^{4a} and R^{5a} are independently selected at each instance from hydrogen, halogen, alkyl, haloalkyl, alkenyl, alkynyl, heterocycle, aryl, and heteroaryl, wherein each alkyl, haloalkyl, alkenyl, alkynyl, heterocycle, aryl, and heteroaryl is optionally substituted as allowed by valence with 1, 2, 3, or 4 substituents selected from R²⁴;
R^{4b} and R^{5b} are independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²⁵;
or R^{4a} and R^{4b} together with the atom to which they are attached are combined to form a spirocycle;
or R^{5a} and R^{5b} together with the atom to which they are attached are combined to form a spirocycle;
R⁶ is hydrogen, cyano, alkyl, haloalkyl, alkenyl, alkynyl, aryl, heterocycle, heteroaryl, —C(O)R⁴⁰, —S(O)R⁴⁰, and —S(O)₂R⁴⁰; each of which alkyl, haloalkyl, alkenyl, alkynyl, aryl, heterocycle, and heteroaryl is optionally substituted as allowed by valence with 1, 2, 3, or 4 substituents selected from R³¹;
each R⁷ and R⁸ is independently selected from hydrogen, alkyl, and haloalkyl;
in certain embodiments R⁷ and R⁸ are both hydrogen;
R¹⁰ is independently selected at each instance from hydrogen, alkyl, haloalkyl, alkenyl, alkynyl, —OR¹¹, —NR¹¹R¹², —SR¹¹, aryl, heterocycle, and heteroaryl; each of which alkyl, haloalkyl, alkenyl, alkynyl, heterocycle, aryl, and heteroaryl is optionally substituted as allowed by valence with 1, 2, 3, or 4 substituents selected from R³⁰;
R¹¹ and R¹² are independently selected at each instance from hydrogen, alkyl, haloalkyl, alkenyl, alkynyl, aryl, heterocycle, heteroaryl, —C(O)R⁴⁰, —S(O)R⁴⁰, and —S(O)₂R⁴⁰; each of which alkyl, haloalkyl, alkenyl, alkynyl, aryl, heterocycle, and heteroaryl is optionally substituted as allowed by valence with 1, 2, 3, or 4 substituents selected from R³¹;
in certain embodiments R¹¹ is CH₂CH₂OH and R¹² is H;
R²¹, R²², R²³, R²⁴, R²⁵, and R²⁶ are independently selected at each instance from hydrogen, halogen, alkyl, haloalkyl, alkenyl, alkynyl, heterocycle, aryl, heteroaryl, cyano, nitro, —C(O)R⁴⁰, —OC(O)R⁴⁰, —NR⁴¹C(O)R⁴⁰, —OR⁴¹, —NR⁴¹R⁴², —S(O)R⁴⁰, —S(O)₂R⁴⁰, —OS(O)R⁴⁰, —OS(O)₂R⁴⁰, —NR⁴¹S(O)R⁴⁰, —NR⁴¹S(O)₂R⁴⁰, and —SR⁴¹, wherein each alkyl, haloalkyl, alkenyl, alkynyl, heterocycle, aryl, and heteroaryl is optionally substituted as allowed by valence with 1, 2, 3, or 4 substituents selected from R⁴³;
R³⁰ and R³¹ are independently selected at each instance from hydrogen, halogen, alkyl, haloalkyl, alkenyl, alkynyl, heterocycle, aryl, heteroaryl, cyano, nitro, —C(O)R⁴⁰, —OC(O)R⁴⁰, —NR⁴¹C(O)R⁴⁰, —OR⁴¹, —NR⁴¹R⁴², —S(O)R⁴⁰, —S(O)₂R⁴⁰, —OS(O)R⁴⁰, —OS(O)₂R⁴⁰, —NR⁴¹S(O)R⁴⁰, —NR⁴¹S(O)₂R⁴⁰, and —SR⁴¹, wherein each alkyl, haloalkyl, alkenyl, alkynyl, heterocycle, aryl, and heteroaryl is optionally substituted as allowed by valence with 1, 2, 3, or 4 substituents selected from R⁴³;
R⁴⁰ is independently selected at each instance from hydrogen, alkyl, haloalkyl, alkenyl, alkynyl, aryl, heterocycle, heteroaryl, amino, hydroxyl, alkoxy, —NHalkyl, and —N(alkyl)₂, each of which except hydrogen is optionally substituted as allowed by valence with 1, 2, 3, or 4 substituents selected from R⁴³;
R⁴¹ and R⁴² are independently selected at each instance from hydrogen, alkyl, haloalkyl, alkenyl, alkynyl, aryl, heterocycle, and heteroaryl; each of which except hydrogen is optionally substituted as allowed by valence with 1, 2, 3, or 4 substituents selected from R⁴³;
R⁴³ is independently selected at each instance from hydrogen, halogen, cyano, nitro, alkyl, haloalkyl, alkenyl, alkynyl, aryl, heterocycle, heteroaryl, amino, hydroxyl, alkoxy, —NHalkyl, —N(alkyl)₂, —OC(O)alkyl, —NHC(O)alkyl, and —N(alkyl)C(O)alkyl;

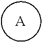

is aryl, heteroaryl, or bicycle;

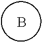

is a bicycle;

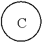

is aryl, heteroaryl, or bicycle;

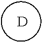

is a heterocycle;

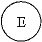

is aryl or heteroaryl; and

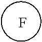

is a heterocycle bonded through a carbon atom.

In certain embodiments the Linker-Ubiquitinated Protein Targeting Ligand replaces a R¹, R², R³, R^{4a}, R^{4b}, R^{5a}, R^{5b}, R⁷, R⁸, R¹⁰, R¹¹, or R¹². In certain embodiments Linker-Ubiquitinated Protein Targeting Ligand is covalent attached to a R¹, R², R³, R^{4a}, R^{4b}, R^{5a}, R^{5b}, R⁷, R⁸, R¹⁰, R¹¹, or R² as allowed by valence. In certain embodiments, the Linker is covalently bound in a position other than R¹, R², R³, R^{4a}, R^{4b}, RSa, R^{5b}, R⁷, R⁸, R¹⁰, R¹¹, or R¹².

In certain embodiments Linker is of Formula:

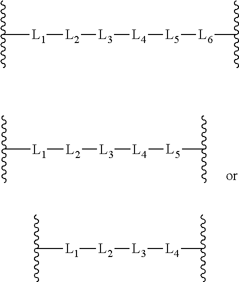

wherein
L₁, L₂, L₃, L₄, L₅, and L₆ are independently selected from the group consisting of a bond, alkyl, alkene, alkyne, haloalkyl, alkoxy, aryl, heterocycle, heteroaryl, bicycle, —C(O)—, —C(O)O—, —OC(O)—, —SO₂—, —S(O)—, —C(S)—, —C(O)NR¹¹—, —NR¹¹C(O)—, —O—, —S—, —NR¹¹—, —P(O)(OR¹¹)O—, —P(O)(OR¹¹)—, polyethylene glycol, lactic acid, and glycolic acid, each of which except bond is optionally substituted with 1, 2, 3, or 4 substituents independently selected from R⁴⁴; wherein Li, L₂, L₃, L₄, L₅, and L₆ are selected such that there are no more than two of the same moieties connected together (e.g, L₁, L₂, and L₃ cannot all three be —C(O)—) and O and N atoms are not directly linked together except within aromatic rings (e.g. L₁ and L₂ cannot both be —O— or NR¹¹);
R⁴⁴ is independently selected at each instance from hydrogen, alkyl, haloalkyl, alkenyl, alkynyl, aryl, heterocycle, heteroaryl, amino, hydroxyl, alkoxy, —NR¹¹R¹², halogen, cyano, nitro, —OC(O)R⁴⁰, —NR¹¹C(O)R⁴⁰, —C(O)R⁴⁰, —OP(O)(R⁴⁰)₂, —P(O)(R⁴⁰)₂, —NR¹¹P(O)(R⁴⁰)₂, —SR¹¹, —OR¹¹, —S(O)R⁴⁰, —S(O)₂R⁴⁰, and —N(alkyl)C(O)R⁴⁰, each of which except hydrogen is optionally substituted as allowed by valence with 1, 2, 3, or 4 substituents selected from R⁴⁵; and
R⁴⁵ is independently selected at each instance from hydrogen, halogen, cyano, nitro, alkyl, haloalkyl, alkenyl, alkynyl, aryl, heterocycle, heteroaryl, amino, hydroxyl, alkoxy, —NHalkyl, —N(alkyl)₂, —OC(O)alkyl, —NHC(O)alkyl, and —N(alkyl)C(O)alkyl.

In certain embodiments the compound of the present invention is of Formula:

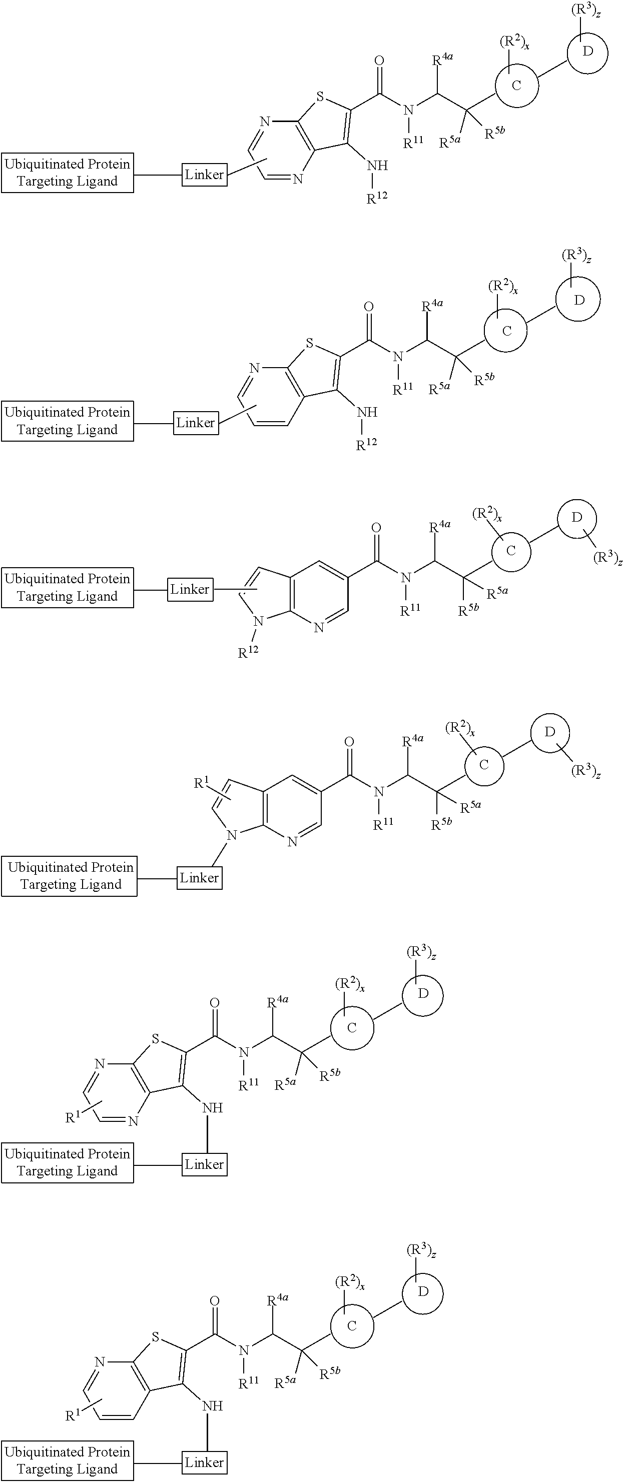

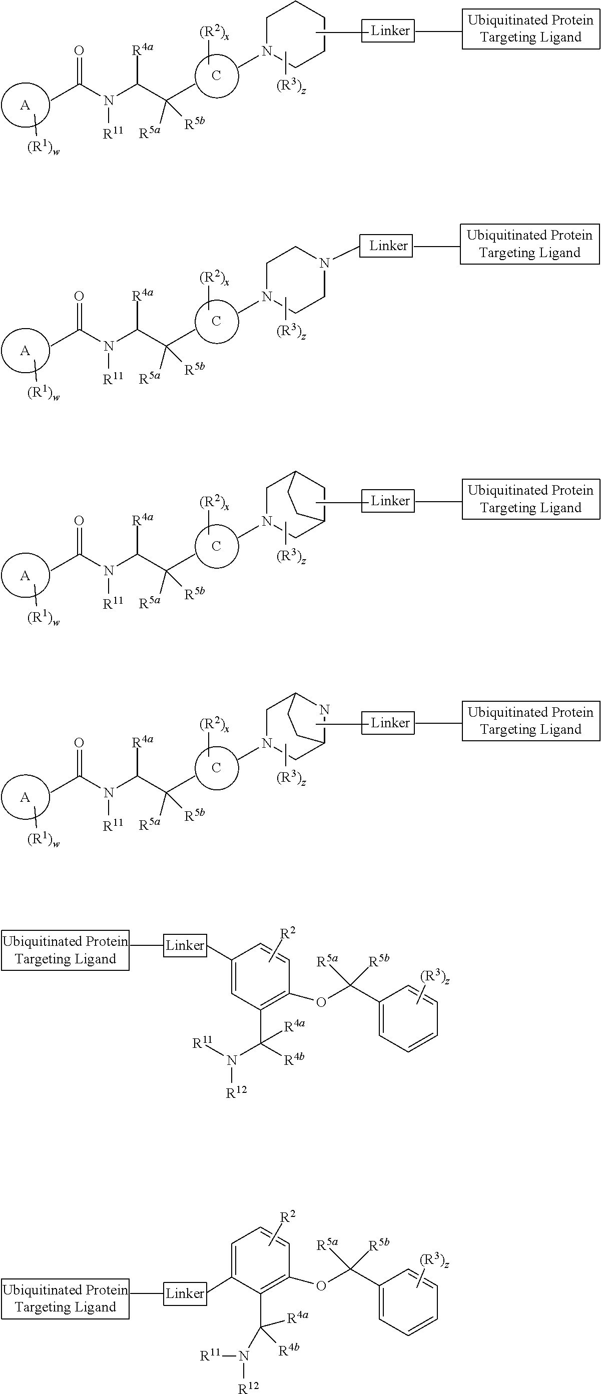

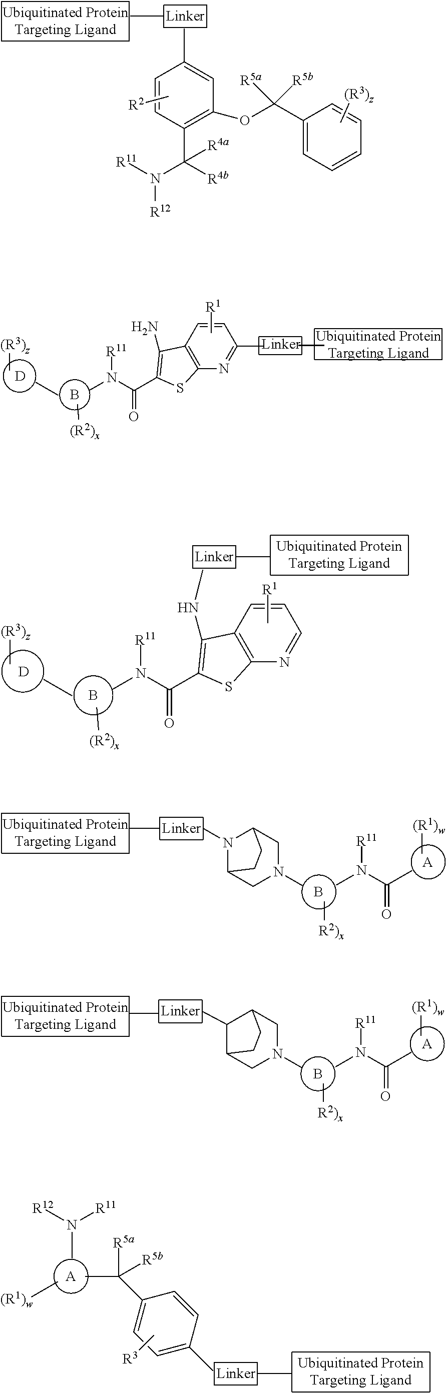

or a pharmaceutically acceptable salt thereof.

In certain embodiments the compound of the present invention is of Formula:

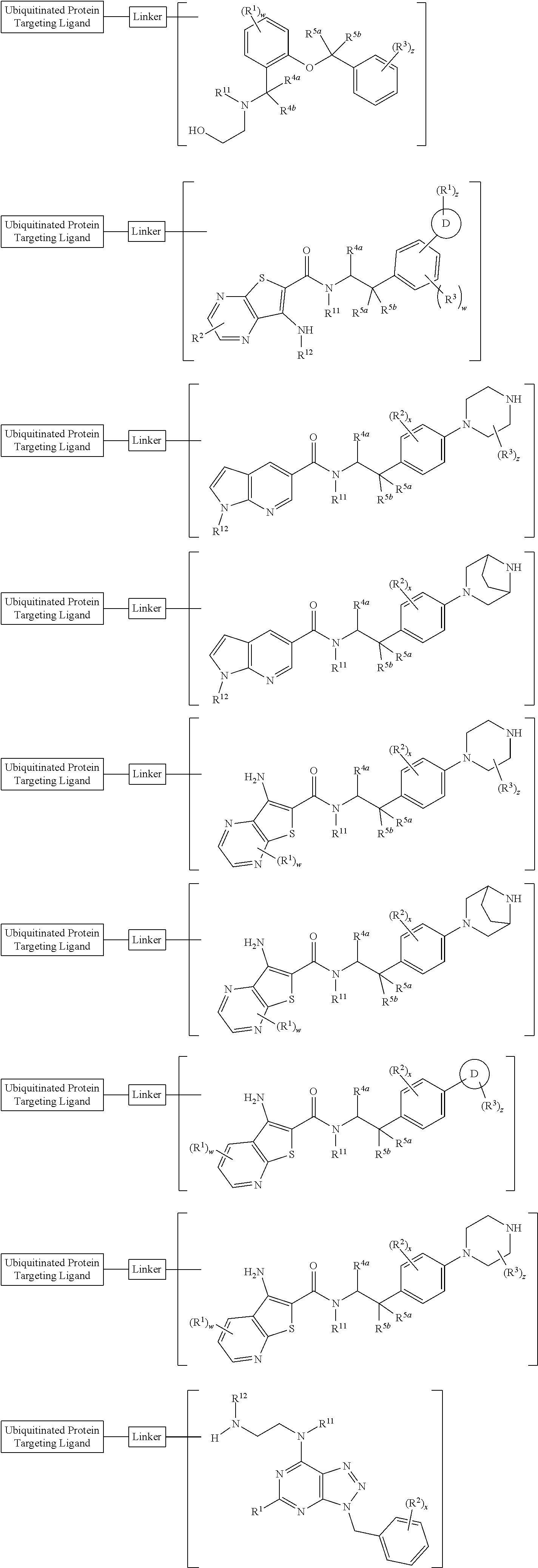

or a pharmaceutically acceptable salt thereof.

In certain aspects a protein stabilizing compound of Formula I is schematically shown as Formula II:

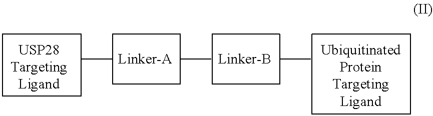

or a pharmaceutically acceptable salt thereof;
wherein
Linker-A is a bivalent moiety that links Linker-B and the USP28 Targeting; and
Linker-B is a bivalent moiety that links the Ubiquitinated Protein Targeting Ligand and Linker-A.

In certain embodiments Linker-A is of Formula:

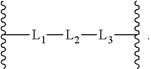

In certain embodiments Linker-B is of Formula:

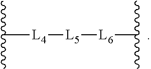

In certain embodiments, the Ubiquitinated Protein Targeting Ligand is a pharmaceutical organic ligand (e.g. not an inorganic substance) that binds to the Target Ubiquitinated Protein adequately to facilitate deubiquitination. In certain embodiments of the invention, the Ubiquitinated Protein Targeting Ligand is a peptide or oligonucleotide that binds to the Target Ubiquitinated Protein adequately to facilitate deubiquitination. In certain embodiments the Ubiquitinated Protein Targeting Ligand is a pharmaceutically active compound or a fragment thereof that binds to the Target Ubiquitinated Protein (for example an approved drug or a compound in development with known binding affinity for the Target Ubiquitinated Protein in either the ubiquitinated or nonubiquitinated form). A plethora of illustrative nonlimiting examples or Ubiquitinated Protein Targeting Ligands for use in the present invention are provided in the Detailed Description and Figures. Additional Ubiquitinated Protein Targeting Ligands are known in the art.

The protein stabilizing compounds described herein stabilize and restore function to a Target Protein by binding and deubiquitinating a Target Ubiquitinated Protein. For example, when the Ubiquitinated Protein Targeting Ligand is an inhibitor of the Target Ubiquitinated Protein then the protein stabilizing compound will deubiquitinate the Target Ubiquitinated Protein and at least partially restore its function, however, the Target Ubiquitinated Protein's activity will not be increased beyond the activity of the non-ubiquitinated version of the protein. In other embodiments a protein stabilizing compound described herein stabilizes, restores, and activates the Target Ubiquitinated Protein. For example, when the Ubiquitinated Protein Targeting Ligand is an agonist or activator of the Target Ubiquitinated Protein then the protein stabilizing compound will deubiquitinate the Target Ubiquitinated Protein, restore its function, and increase its activity.

By restoring function to proteins which have beneficial activity the compounds described herein can be used to treat a variety of difficult to treat disorders. Non-limiting examples of Target Ubiquitinated Proteins include RIPK1, BRD7, c-Myc, rhodopsin, p53, PAH, CFTR, MSH2, PDCD4, p27-kip1, ABCA4, and ABCB11-4 or a mutant form, splice variant, or altered sequence thereof. Additional examples of Target Ubiquitinated Proteins include KEAP1, PKLR, KCNQ1, TK2, STING1, IRAK4, PTEN, SERPINA1, P21, BAX, and RIPK2 or a mutant form, splice variant, or altered sequence thereof. In certain embodiments, a method of treating a disorder mediated by a Target Ubiquitinated Protein is provided comprising administering an effective amount of a protein stabilizing compound described herein, or a pharmaceutically acceptable salt thereof, to a patient in need thereof, for example a human, optionally in a pharmaceutically acceptable carrier. For example, in certain embodiments, a protein stabilizing compound of Formula I or Formula II, is administered to a human to treat a cancer or tumor where the protein stabilizing compound has a Ubiquitinated Protein Targeting Ligand that binds the Target Ubiquitinated Protein, and the tumor or cancer is mediated by the Target Ubiquitinated Protein.

In certain embodiments the Target Ubiquitinated Protein is ChAT (for example P17A/P19A mutant ChAT), CYLD (for example missense mutant CYLD), NEMO, AIP (for example missense AIP or nonsense mutant AIP), or Eyal (for example S454P, L472R, or L550P Eyal).

Non-limiting examples of disorders that can be treated by a protein stabilizing compound of the present invention include inflammation (for example wherein the compound stabilizes RIPK2 or a mutant thereof), a cancer (for example wherein the compound stabilizes BAX, PTEN, or KEAP1), pulmonary emphysema (for example wherein the compound stabilizes alpha antitrypsin (SERPINA1) or a mutant thereof), immunodeficiency (IRAK4, STING1), mitochondrial depletion syndrome (TK2), pituitary hormone deficiency (KCNQ1)

Additional non-limiting examples of disorders that can be treated by a protein stabilizing compound of the present invention include cystic fibrosis (for example wherein the compound stabilizes CFTR or a mutant thereof), phenylketonuria (for example wherein the compound stabilizes PAH or a mutant thereof), progressive familial intrahepatic cholestasis (for example wherein the compound stabilizes ABCB11/4 or a mutant thereof), Stargardt Disease (for example wherein the compound stabilizes ABCA4 or a mutant thereof), retinitis pigmentosa (for example wherein the compound stabilizes rhodopsin or a mutant thereof), a cancer (for example wherein the compound stabilizes p53, cMyc, P27^{KiP1}, PDCD4, MSH2, or RIPK1 or a mutant thereof), congenital myasthenic syndrome (for example wherein the compound stabilizes ChAT or a mutant thereof), Brooke-Spiegler syndrome (for example wherein the protein stabilizes CYLD or NEMO or a mutant thereof), pituitary adenoma (for example wherein the compound stabilizes AIP or a mutant thereof), or BOR syndrome (for example wherein the protein stabilizes Eyal or a mutant thereof).

A protein stabilizing compound of the present invention can be administered in any manner that allows the compound to stabilize the Target Ubiquitinated Protein and/or restore its function. As such, examples of methods to deliver the protein stabilizing compound of the present invention include, but are not limited to, systemic, parenteral, topical, oral, intravenous, buccal, sublingual, subcutaneous, or transnasal administration.

In certain embodiments, the protein stabilizing compound of the present invention has at least one desired isotopic substitution of an atom, at an amount above the natural abundance of the isotope, i.e., enriched.

In one embodiment, the protein stabilizing compound of the present invention includes a deuterium or multiple deuterium atoms.

Another aspect of the present invention provides a protein stabilizing compound as described herein, or an enantiomer, diastereomer, or stereoisomer thereof, or pharmaceutically acceptable salt, hydrate, or solvate thereof, or a pharmaceutical composition, for use in the manufacture of a medicament for treating or preventing a disease in which the Target Ubiquitinated Protein plays a role.

In certain embodiments a method of stabilizing and restoring a protein's function is provided. The skilled artisan will recognize how to assess whether or not a protein's function has been restored in vivo or in vitro depending on context. For example, when the Target Ubiquitinated Protein is an ion channel, such as CFTR, surface representation assays or ion current assays can be used to assay protein function restoration in vitro. Additionally, a reduction of symptoms associated with a disease mediated by the Target Ubiquitinated Protein will show in vivo efficacy. For example, when the Target Ubiquitinated Protein is CFTR amelioration of cystic fibrosis symptoms will result from protein function restoration in vivo. When the Target Ubiquitinated Protein is an oncological target, such as p53, cell death assays or cell cycle assays can be used to demonstrate the restoration of function. When the Target Ubiquitinated Protein is an enzyme then its enzymatic activity can be assayed to demonstrate the restoration of function.

The Target Ubiquitinated Protein can be in a prokaryotic cell or a eukaryotic cell, including but not limited to eukaryotic cells in multicellular organisms. In certain embodiments the Target Ubiquitinated Protein is in a eukaryotic cell in an animal, including but not limited to humans.

Other features and advantages of the present application will be apparent from the following detailed description.

The present invention thus includes at least the following features:
(a) A protein stabilizing compound of Formula I or Formula II as described herein, or a pharmaceutically acceptable salt or isotopic derivative (including a deuterated derivative) thereof;
(b) A method for treating a disorder mediated by a Target Ubiquitinated Protein, comprising administering an effective amount of a protein stabilizing compound of Formula I or Formula II, or pharmaceutically acceptable salt thereof, as described herein, to a patient in need thereof wherein the protein stabilizing compound contains a Ubiquitinated Protein Targeting Ligand that binds the Target Ubiquitinated Protein;
(c) A protein stabilizing compound of Formula I or Formula II, or a pharmaceutically acceptable salt thereof for use in the treatment of a disorder that is mediated by a Target Ubiquitinated Protein, wherein the protein stabilizing compound contains a Ubiquitinated Protein Targeting Ligand that binds the Target Ubiquitinated Protein;
(d) Use of a protein stabilizing compound of Formula I or Formula II, or a pharmaceutically acceptable salt thereof, in an effective amount in the treatment of a patient in need thereof, typically a human, with disorder mediated by a Target Ubiquitinated Protein, wherein the protein stabilizing compound contains a Ubiquitinated Protein Targeting Ligand that binds the Target Ubiquitinated Protein;
(e) Use of a protein stabilizing compound of Formula I or Formula II, or a pharmaceutically acceptable salt thereof in the manufacture of a medicament for the treatment of a disorder mediated by a Ubiquitinated Protein Targeting Ligand that binds the Target Ubiquitinated Protein;
(f) A pharmaceutical composition comprising a protein stabilizing compound of Formula I or Formula II, or a pharmaceutically acceptable salt thereof; and a pharmaceutically acceptable carrier or diluent;
(g) A protein stabilizing compound of Formula I or Formula II, as described herein as a mixture of enantiomers or diastereomers (as relevant), including as a racemate;
(h) A protein stabilizing compound of Formula I or Formula II, as described herein in enantiomerically or diastereomerically (as relevant) enriched form, including an isolated enantiomer or diastereomer (i.e., greater than 85, 90, 95, 97, or 99% pure); and
(i) A process for the preparation of therapeutic products that contain an effective amount of a protein stabilizing compound of Formula I or Formula II, or a pharmaceutically acceptable salt thereof, as described herein.

### BRIEF DESCRIPTION OF THE FIGURES

As used in the figures:
y is 0, 1, 2, or 3;
yy is 0, 1, 2, or 3;
R⁹⁹ is the attachment point to Linker-Ubiquitinated Protein Targeting Ligand;
R¹⁰⁰ is the attachment point to Linker-USP28 Targeting Ligand;
R²⁰⁰ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²¹.

As used herein, where a cyclic group within a drawn molecule has a number in the middle of the cycle these numbers are used to denote cycles to which the Linker may be attached as allowed by valence.

In certain embodiments the Linker is attached to the cycle marked with a 1.

In certain embodiments the Linker is attached to the cycle marked with a 2.

In certain embodiments the Linker is attached to the cycle marked with a 3.

In certain embodiments the Linker is attached to the cycle marked with a 4.

In certain embodiments the Linker is attached to the cycle marked with a 5.

In certain embodiments the Linker is attached to the cycle marked with a 6.

In certain embodiments the Linker is attached to the cycle marked with a 7.

For example

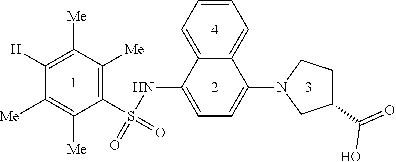

when attached to the Linker in the cycle marked with a 1 includes the following non-limiting exemplary structure:

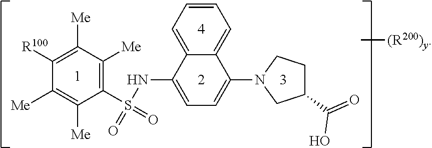

Where a substituent is already on the cycle marked 1, 2, 3, 4, 5, or 6, the linker may be on or replace that substituent as allowed by valence. For example

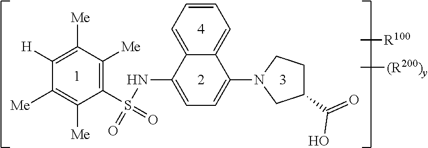

when attached to the Linker in the cycle marked with a 1 also includes the following non-limiting exemplary structures:

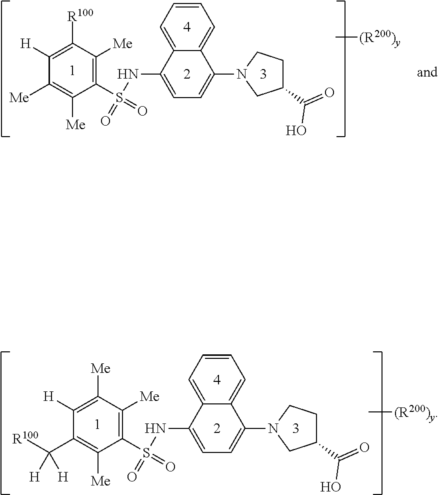

FIG. 1A, FIG. 1B, FIG. 1C, FIG. 1D, FIG. 1E, FIG. 1F, FIG. 1G, FIG. 1H and FIG. 1I present non-limiting examples of ligands that bind to Ubiquitin Specific Peptidase 28 (USP28). Additional non-limiting examples and related ligands, are identified in “Identification and characterization of dual inhibitors of the USP25/28 deubiquitinating enzyme subfamily” ACS Chem. Biol. 12, 3113-3125 (2017); “Discovery of [1,2,3]triazolo[4,5-d]pyrimidine derivatives as highly potent, selective, and cellularly active USP28 inhibitors” Acta Pharm Sinica B 10, 1476-1491 (2020); USP28 deletion and small molecule inhibition destabilizes c-Myc and elicits regression of squamous cell lung carcinoma” Biorxiv 2020.11.17.377705 (2020) doi:10.1101/2020.11.17.377705; WO 2020/2246524; WO 2019/032863; CN 111909181; and CN 112898314.

FIG. 2A, FIG. 2B, FIG. 2C, and FIG. 2D present non-limiting examples of ligands that bind to Cystic fibrosis transmembrane conductance regulator (CFTR), including the compounds LIP, CLR, AJP, VX7, POV, FSC, AP5, 4HY, A99, 64N, 64L, and 640. For additional non-limiting examples and related ligands, see ligands identified by Liu, F., et al., “Structural identification of a hotspot on CFTR for potentiation”, Science, 2019, 364: 1184-1188; Stevers, L. M., et al., “Characterization and small-molecule stabilization of the multisite tandem binding between 14-3-3 and the R domain of CFTR”, Proc Natl Acad Sci USA, 2016,113: El 152-El 161; Lammens, A., Hopfner, K. P., “Structural Basis for Adenylate Kinase Activity in ABC ATPases”, J Mol Biol., 2010, 401: 265-273; Bahl, C. D., et al., “ ”, Angew Chem Int Ed Engl., 2015, 54: 9881-9885; Voellmecke, C., et al., “Conformational Changes in the Catalytic Domain of the Cpx-ATPase Copb-B Upon Nucleotide Binding”, to be published; Kitamura, S., et al., “Rational Design of Potent and Selective Inhibitors of an Epoxide Hydrolase Virulence Factor from Pseudomonas aeruginosa”, J Med Chem., 2016, 59: 4790-4799; Ridley K, et al., “Elexacaftor-Tezacaftor-Ivacaftor: The First Triple-Combination Cystic Fibrosis Transmembrane Conductance Regulator Modulating” Therapy. J Pediatr Pharmacol Ther. 2020; 25(3):192-197; Ghelani et al., “Emerging Cystic Fibrosis Transrnembrane Conductance Regulator Modulators as New Drugs for Cystic Fibrosis: A Portrait of in Vitro Pharmacology and Clinical Translation” ACS Pharmacol. Transl. Sci. 2020, 3, 1, 4-10; Fiedorczuk K, et al., “Mechanism of CFTR Correction by Type I Folding Correctors, bioRxiv prepring 2021, doi.org/10.1101/2021.06.18.449063; Grand et al., “Discovery of Icenticaftor (GBW251), a Cystic Fibrosis Transmembrane Conductance Regulator Potentiator with Clinical Efficacy in Cystic Fibrosis and Chronic Obstructive Pulmonary Disease” J. Med. Chem 2021, 64, 11, 7241-7260; Plas et al.; “Discovery of GLPG2451, a Novel Once Daily Potentiator for the Treatment of Cystic Fibrosis” J. Med. Chem. 2021, 64, 1, 343-353; Hadida et al., “Discovery of N-(2,4-Di-tert-butyl-5-hydroxyphenyl)-4-oxo-1,4-dihydroquinoline-3-carboxamide (VX-770, Ivacaftor), a Potent and Orally Bioavailable CFTR Potentiator” J. Med. Chem. 2014, 57, 23, 9776-9795; Hughes “Patent Review of Synthetic Routes and Crystalline Forms of the CFTR-Modulator Drugs Ivacaftor, Lumacaftor, Tezacaftor, and Elexacaftor” Org. Process Res. Dev. 2019, 23, 11, 2302-2322. Plas et al., “Discovery of N-(3-Carbamoyl-5,5,7,7-tetramethyl-5,7-dihydro-4H-thieno[2,3-c]pyran-2-yl)-IH-pyrazole-5-carboxamide (GLPG1837), a Novel Potentiator Which Can Open Class III Mutant Cystic Fibrosis Transmembrane Conductance Regulator (CFTR) Channels to a High Extent” J. Med. Chem. 2018, 61, 4, 1425-1435; Wang et al., “Discovery of 4-[(2R,4R)-4-({[1-(2,2-Difluoro-1,3-benzodioxol-5-yl)cyclopropyl]carbonyl}amino)-7-(difluoromethoxy)-3,4-dihydro-2H-chromen-2-yl]benzoic Acid (ABBV/GLPG-2222), a Potent Cystic Fibrosis Transmembrane Conductance Regulator (CFTR) Corrector for the Treatment of Cystic Fibrosis” J. Med. Chem. 2018, 61, 4, 1436-1449.

FIG. 3A, FIG. 3B, and FIG. 3C present non-limiting examples of ligands that bind to Phenylalanine Hydroxylase (PAH) including the compounds PHE, HBI, 3QI, H4B, TIH, H2B, XDE, LNR, LDP, DAH, and PIN. For additional non-limiting examples and related ligands, see ligands identified by Ronau et al., “An additional substrate binding site in a bacterial phenylalanine hydroxylase”, Eur Biophys J., 2013, 42: 691-708; Erlandsen et al., “Structural comparison of bacterial and human iron-dependent phenylalanine hydroxylases: similar fold, different stability and reaction rates”, J Mol Biol., 2002, 320: 645-661; Torreblanca et al., “Structural and Mechanistic Basis of the Interaction between a Pharmacological Chaperone and Human Phenylalanine Hydroxylase”, Chembiochem., 2012, 13: 1266; Anderson et al., “Crystal Structure of the Ternary Complex of the Catalytic Domain of Human Phenylalanine Hydroxylase with Tetrahydrobiopterin and 3-(2-thienyl)-L-alanine, and its Implications for the Mechanism of Catalysis and Substrate Activation”, J Mol Biol., 2002, 320: 1095-1108; Erlandsen et al., “Correction of kinetic and stability defects by tetrahydrobiopterin in phenylketonuria patients with certain phenylalanine hydroxylase mutations”, Proc Natl Acad Sci USA, 2004, 101: 16903-16908; Erlandsen et al., “Crystallographic analysis of the human phenylalanine hydroxylase catalytic domain with bound catechol inhibitors at 2.0 A resolution”, Biochemistry, 1998m 37: 15638-15646; Zhuang et al., “Phenylalanine hydroxylase from dictyostelium—BH2 complex”, to be published; Perchik et al., “The Effects of Ligand Deprotonation on the Binding Selectivity of the Phenylalanine Hydroxylase Active Site” Computation and Theoretical Chemistry, 2019, 1153, 19-24.

FIG. 4A, FIG. 4B, and FIG. 4C present non-limiting examples of ligands that bind to Tumor protein P53 (p5³). For additional non-limiting examples and related ligands, see ligands identified by Baud et al., “Aminobenzothiazole derivatives stabilize the thermolabile p53 cancer mutant Y220C and show anticancer activity in p53-Y220C cell lines”, Eur J Med Chem., 2018, 152: 101-114; Allen et al., “Discovery and optimization of chromenotriazolopyrimidines as potent inhibitors of the mouse double minute 2-tumor protein 53 protein-protein interaction”, J Med Chem., 2009, 52: 7044-7053; Bauer et al., “A structure-guided molecular chaperone approach for restoring the transcriptional activity of the p53 cancer mutant Y220C”, Future Med Chem., 2019, 11: 2491-2504; Boeckler et al., “Targeted Rescue of a Destabilized Mutant of P53 by an in Silico Screened Drug”, Proc Natl Acad Sci USA, 2008, 105: 10360; Liu et al., “Small molecule induced reactivation of mutant p53 in cancer cells”, Nucleic Acids Res., 2013, 41: 6034-6044; Wilcken et al., “Halogen-Enriched Fragment Libraries as Leads for Drug Rescue of Mutant P53”, J Am Chem Soc., 2012, 134: 6810; Bauer et al., “Harnessing Fluorine-Sulfur Contacts and Multipolar Interactions for the Design of P53 Mutant Y220C Rescue Drugs”, ACS Chem Biol., 2016, 11: 2265; Joerger et al., “Exploiting Transient Protein States for the Design of Small-Molecule Stabilizers of Mutant P53”, Structure, 2015, 23: 2246; Basse et al., “Toward the Rational Design of p53-Stabilizing Drugs: Probing the Surface of the Oncogenic Y220C Mutant”, Chemistry and Biology, 2010, 29, 46-56.

FIG. 5A and FIG. 5B presents non-limiting examples of ligands that bind to Rhodopsin including the compounds DOK, DNZ, DO5, DL2, DLB, DLH, DN5, and 7AB. For additional non-limiting examples and related ligands, see ligands identified by Murakami et al., “Crystallographic Analysis of the Primary Photochemical Reaction of Squid Rhodopsin”, J Mol Biol., 2011, 413: 615-627; Okada et al., “Functional role of internal water molecules in rhodopsin revealed by X-ray crystallography”, Proc Natl Acad Sci USA, 2002, 99: 5982-5987; Mattle et al., “Ligand channel in pharmacologically stabilized rhodopsin”, Proc Natl Acad Sci USA., 2018, 115: 3640-3645; Gulati et al., “Photocyclic behavior of rhodopsin induced by an atypical isomerization mechanism”, Proc Natl Acad Sci USA, 2017, 114: E2608-E2615, Zhou et al. “Structure and Activation of Rhodopsin”, Acta Pharmacol Sin. 2020, 33, 291-299.

FIG. 6A and FIG. 6B present non-limiting examples of ligands that bind to c-Myc including the compounds QUL, 9WP, B06, QUE, Q8P, Q8D, Q8G, Q8S, Q8M, and QF1. For additional non-limiting examples and related ligands, see ligands identified by Dai et al., “Solution Structure of a 2:1 Quindoline-c-MYC G-Quadruplex: Insights into G-Quadruplex-Interactive Small Molecule Drug Design”, J Am Chem Soc., 2011, 133: 17673-17680; Calabrese et al., “Chemical and structural studies provide a mechanistic basis for recognition of the MYC G-quadruplex”, Nat Commun., 2018, 9: 4229-4229; Liu et al., “Structures of 1:1 and 2:1 complexes of BMVC and MYC promoter G-quadruplex reveal a mechanism of ligand conformation adjustment for G4-recognition”, Nucleic Acids Res., 2019, 47: 11931-11942; Kumar et al., “Solution structure for quercetin complexed with c-myc G-quadruplex DNA”, to be published; Chacon Simon et al., “Discovery of WD Repeat-Containing Protein 5 (WDR5)-MYC Inhibitors Using Fragment-Based Methods and Structure-Based Design”, J Med Chem., 2020, 63: 4315-4333; Whitefield et al., “Strategies to Inhibit Myc and Their Clinical Applicability” Front Cell Dev. Biol., 2017, 5, 10.

FIG. 7A, FIG. 7B, FIG. 7C, FIG. 7D, and FIG. 7E present non-limiting examples of ligands that bind to Receptor-interacting protein kinase 1 (RIPK1 or RIP1 kinase) including the compounds L4Y, L8D, NAG, UDP, EJP, EJY, LN4, QOK, RCM, 1HW, 1HX, Q1A, 65U, M5J, JSW, 7MJ, K8K, and G4W. For additional non-limiting examples and related ligands, see ligands identified by Hamilton et al., “Potent and selective inhibitors of receptor-interacting protein kinase 1 that lack an aromatic back pocket group”, Bioorg Med Chem Lett., 2019, 29: 1497-1501; Patel et al., “RIP1 inhibition blocks inflammatory diseases but not tumor growth or metastases”, Cell Death Differ., 2020, 27: 161-175; Ding et al., “Structural and Functional Insights into Host Death Domains Inactivation by the Bacterial Arginine GlcNAcyltransferase Effector”, Mol Cell, 2019, 74: 922; Yoshikawa et al., “Discovery of 7-Oxo-2,4,5,7-tetrahydro-6H-pyrazolo[3,4c]pyridine Derivatives as Potent, Orally Available, and Brain-Penetrating Receptor Interacting Protein 1 (RIP1) Kinase Inhibitors: Analysis of Structure-Kinetic Relationships”, J Med Chem., 2018, 61: 2384-2409; Pierotti et al., “Potent Inhibition of Necroptosis by Simultaneously Targeting Multiple Effectors of the Pathway”, ACS Chem Biol., 2020, 15: 2702-2713; Rubbelke et al., “Locking mixed-lineage kinase domain-like protein in its auto-inhibited state prevents necroptosis”, Proc Natl Acad Sci USA, 2020, 117: 33272-33281; Xie et al., “Structural Basis of RIP1 Inhibition by Necrostatins”, Structure, 2013, 21: 493-499; Harris et al., “Discovery of Small Molecule RIP1 Kinase Inhibitors for the Treatment of Pathologies Associated with Necroptosis”, ACS Med Chem Lett., 2013, 4: 1238-1243; Harris et al., “DNA-Encoded Library Screening Identifies Benzo[b][1,4]oxazepin-4-ones as Highly Potent and Monoselective Receptor Interacting Protein 1 Kinase Inhibitors”, J Med Chem., 2016, 59: 2163-2178; Harris et al., “Discovery and Lead-Optimization of 4,5-Dihydropyrazoles as Mono-Kinase Selective, Orally Bioavailable and Efficacious Inhibitors of Receptor Interacting Protein 1 (RIP1) Kinase”, J Med Chem., 2019, 62: 5096-5110; Harris et al., “Discovery of a First-in-Class Receptor Interacting Protein 1 (RIP1) Kinase Specific Clinical Candidate (GSK2982772) for the Treatment of Inflammatory Diseases”, J Med Chem., 2017, 60: 1247-1261; Harris et al., “Identification of a RIP1 Kinase Inhibitor Clinical Candidate (GSK3145095) for the Treatment of Pancreatic Cancer”, ACS Med Chem Lett, 2019, 10: 857-862; Wang et al., “RIP1 Kinase Drives Macrophage-Mediated Adaptive Immune Tolerance in Pancreatic Cancer”, Cancer Cell, 2018, 34: 757-774.e7.

FIG. 8 presents non-limiting examples of ligands that bind to DNA mismatch repair protein Msh2 (MSH2, MutS protein homolog 2) in the MSH2-MSH6 complex, including the ligands identified in Vasilyeva et al. DNA Repair, 2009, 8(1): 103-113 and Nair et al. Nucleic Acids Res., 2018, 42: 256-266.

FIG. 9A and FIG. 9B present non-limiting examples of ligands that bind to Cyclin-dependent kinase inhibitor 1B (Cyclin-dependent kinase inhibitor p27, CDKN1B, p27Kip1). For additional non-limiting examples and related ligands, see ligands identified by Frankel et al. J. Biol. Chem. 2008, 283(2): 1026-1033 and Iconaru et al. Sci. Rep. 2015, 5: 15686.

FIG. 10 presents a non-limiting example of a ligand that binds to retinal-specific phospholipid-transporting ATPase ABCA4 (ABCA4, RTM ABC transporter, ATP-binding cassette sub-family A member 4, Stargardt disease protein) including AJP and CLR. For additional non-limiting examples and related ligands, see Liu et al. eLife, 2021, 10: e63524.

FIG. 11A and FIG. 11B present non-limiting examples of ligands that bind to bile salt export pump (ABCB 11, ATP-binding cassette sub-family B member 11). For additional non-limiting examples and related ligands, see ligands identified by Ritschel et al., Chem. Res. Toxicol., 2014, 27, 873-881 and Jain et al. J. Comput. Aided Mol. Des. 2017, 31(6): 507-521.

FIG. 12 presents non-limiting examples of ligands that bind to Choline O-acetyltransferase (ChAT, choline acetylase, CHOACTase), including the compound RMW. For additional non-limiting examples and related ligands, see ligands identified by Wiktelius et al. Angew. Chem. Int. Ed. 2021, 60(2): 813-819 and Kim et al. Biochemistry, 2006, 45(49), 14621-14631.

FIG. 13 presents a non-limiting example of a ligand that binds to ubiquitin carboxyl-terminal hydrolyase CYLD (CYLD, deubiquitinating enzyme CYLD, ubiquitin-specific-processing protease CYLD), as identified in Yamanaka et al. Biochem. Biophys. Res. Commun., 2020, 524(1): 1-7.

FIG. 14 presents non-limiting examples of ligands that bind to NF-kappa-B essential modulator (NEMO, FIP-3, IkB kinase-associated protein 1, IKKAP1, IKKG). For additional non-limiting examples and related ligands, see ligands identified by Vincendeau et al., Sci. Rep., 2016, 6: 1894 and De Falco et al. Biochemical Pharmacology, 2016, 104: 83-94.

FIG. 15A and FIG. 15B present non-limiting examples of ligands that bind to AH receptor-interacting protein (AIP, Aryl-hydrocarbon receptor-interacting protein, HBV X-associated protein 2). For additional non-limiting examples and related ligands, see ligands identified by Schmees et al. AACR Annual Meeting 2019, Atlanta, GA, Boitano et al., Science, 2010, 329(5997): 1345-1348, Fukuda et al., Biochem. Biophys. Res. Commun., 2007, 359(3): 822-827, Mukai et al., Archives of Biochemistry and Biophysics, 2010, 501: 134-141, and Smith et al., J. Investig. Dermatol., 2017, 137(10): 2110-2119.

FIG. 16 presents non-limiting examples of ligands that binds to programmed cell death protein 4 (PDCD4). For additional non-limiting examples and related ligands, see ligands identified in Frankel et al., J. Biol. Chem. 2008, 283(2): 1026-1033 and Wang et al., “Targeting Programmed Cell Death 4 (PDCD4) with Biogenic Compounds in ARDS by Gaussian Process-Based QSAR Virtual Screening” Journal of Chemometrics 2016, 30: 621-627.

FIG. 17A, FIG. 17B, FIG. 17C and FIG. 17D present non-limiting examples of ligands that binds to Receptor-interacting serine/threonine-protein kinase 2 (RIPK2) including OLI, E7N, 9WS, 9XA, BW8, KRE, GEZ, Q9J, M5W, M2B, 6GD, 6GE, K9T, KA2, SB2, IQ7, ACP, XYW, and SR8. For additional non-limiting examples and related ligands, see ligands identified in Hrdinka et al. Small molecule inhibitors reveal an indispensable scaffolding role of RIPK2 in NOD2 signaling. (2018) EMBO J 37. He et al. Identification of Potent and Selective RIPK2 Inhibitors for the Treatment of Inflammatory Diseases. (2017) ACS Med Chem Lett 8: 1048-1053. Canning et al. Inflammatory Signaling by NOD-RIPK2 Is Inhibited by Clinically Relevant Type II Kinase Inhibitors. (2015) Chem Biol 22: 1174-1184. Suubsuwong, et al. Activation loop targeting strategy for design of receptor-interacting protein kinase 2 (RIPK2) inhibitors. (2018) Bioorg Med Chem Lett 28: 577-583. Suebsuwong, et al. Design of 3,5-diaryl-2-aminopyridines as receptor-interacting protein kinase 2 (RIPK2) and nucleotide-binding oligomerization domain (NOD) cell signaling inhibitors. Unpublished. Haile, et al. Identification of Quinoline-Based RIP2 Kinase Inhibitors with an Improved Therapeutic Index to the hERG Ion Channel. (2018) ACS Med Chem Lett 9: 1039-1044. Haffner, et al. Discovery of Pyrazolocarboxamides as Potent and Selective Receptor Interacting Protein 2 (RIP2) Kinase Inhibitors. (2019) ACS Med Chem Lett 10: 1518-1523. Pellegrini, et al. Structures of the inactive and active states of RIP2 kinase inform on the mechanism of activation. (2017) PLoS One 12: e0177161-e0177161. Charnley, et al. Crystal Structures of Human Rip2 Kinase Catalytic Domain Complexed with ATP-Competitive Inhibitors: Foundations for Understanding Inhibitor Selectivity. (2015) Bioorg Med Chem 23: 7000.

FIG. 18A FIG. 18B and FIG. 18C. present non-limiting examples of ligands that binds to apoptosis regulator BAX. For additional non-limiting examples and related ligands, see Li et. al U.S. Pat. No. 9,561,215, Halazy, et al. Preparation of 9-(piperazinylalkyl) carbazoles as Bax-modulators WO2001/029028. Halazy et al, Synthesis of substituted N-acyl/sulfonyl pyrrolidine derivatives as bax inhibitors. WO2001/072705A1. Halazy, et al. Preparation of pyrrolidines as inhibitors of Bax function. WO2001/074769A1. Xingming et al. Preparation of fluoren-9-ylidenemethylpyridine derivatives as Bax agonists WO2013/028543A1. Walensky et al. Preparation of pyrazol-3-ones as activators of pro-apoptotic BAX. WO2013055949A2. Gavathiotis, et al. Direct and selective small-molecule activation of proapoptotic BAX. Nature Chemical Biology 8, 639-645 (2012). Garner et al. Small-molecule allosteric inhibitors of BAX. Nat Chem Biol 15, 322-330 (2019). Stornaiuolo et al. Structure-Based Lead Optimization and Biological Evaluation of BAX Direct Activators as Novel Potential Anticancer Agents J. Med. Chem. 2015, 58, 5, 2135-2148. Spitz et al. Eltrombopag directly inhibits BAX and prevents cell death. Nature Communications 12, 1134 (2021). Reyna et al. Direct Activation of BAX by BTSA1 Overcomes Apoptosis Resistance in Acute Myeloid Leukemia. Cancer Cell 32, 490-505.e10 (2017).

FIG. 19A and FIG. 19B present non-limiting examples of ligands that bind to P21 (CDKN1A, P21Cip1/Waf1, CAP20, Cyclin-Dependent Kinase Inhibitor 1A). For additional non-limiting examples and related ligands, see Weiss et al. US 2015/0132408, Weiss et al. WO 2014/007998, Park et al. High throughput screening of a small molecule one-bead-one-compound combinatorial library to identify attenuators of p21 as chemotherapy sensitizers. Cancer Biology & Therapy, (7), 12, 2015-2022, and Weiss et al. US 2011/0301192.

FIG. 20 presents a non-limiting example of ligands that bind to alpha-i-antitrypsin (AAT, SERPINA1). For additional non-limiting examples, see Smith et al. WO2019/243841. Mallya et al. Small Molecules Block the Polymerization of Z al-Antitrypsin and Increase the Clearance of Intracellular Aggregates. J. Med. Chem. (2007), 50(22), 5357-5363. Patschull, et al. In silico assessment of potential druggable pockets on the surface of al-antitrypsin conformers PLoS One (2012), 7(5), e36612 FIGS. 21A, 21B, and 21C present non-limiting examples of ligands that bind to pyruvate kinase liver/red blood cell (Pyruvate kinase L/R, PKLR). For additional non-limiting examples, see WO 2019/035863, WO 2019/035863, WO2020198067, and WO2019/075367.

FIG. 22 presents a non-limiting example of ligands that bind to Kelch-like ECH-associated protein 1 (KEAP1). For additional non-limiting examples, see Tran et al. A Comparative Assessment Study of Known Small-Molecule Keapl-Nrf2 Protein-Protein Interaction Inhibitors: Chemical Synthesis, Binding Properties, and Cellular Activity. J Med Chem 62, 8028-8052 (2019).

FIG. 23 presents a non-limiting example of ligands that bind to Phosphatase and Tensin Homolog (PTEN). For additional non-limiting examples, see Li et al. Pretreatment with phosphatase and tensin homolog deleted on chromosome 10 (PTEN) inhibitor SF1670 augments the efficacy of granulocyte transfusion in a clinically relevant mouse model. Blood (2011) 117 (24): 6702-6713.

FIG. 24 presents a non-limiting example of ligands that bind to Interleukin 1 Receptor Associated Kinase 4 (IRAK4). For additional non-limiting examples, see McElroy, W. T. Interleukin-1 receptor-associated kinase 4 (IRAK4) inhibitors: an updated patent review (2016-2018). Expert Opin Ther Pat 29, 243-259 (2019); Lee et al. J. Med. Chem. 2017, 60, 13, 5521-5542, WO 2017205762A1, WO 2017205766A1, WO 2017205769A1

FIG. 25A and FIG. 25B present non-limiting examples of ligands that bind to Thymidine kinase 2, mitochondrial (TK2). For additional non limiting examples, see Van Poeke et al. 3′-[4-Aryl-(1,2,3-triazol-1-yl)]-3′-deoxythymidine Analogues as Potent and Selective Inhibitors of Human Mitochondrial Thymidine Kinase J. Med. Chem. 2010, 53, 7, 2902-2912; Kierdaszuk et al. Substrate/Inhibitor Properties of Human Deoxycitidine Kinase (dCK) and Thymidine Kinases (Tk1 and Tk2) Towards the Sugar Moiety of Nucleosides, Including O′-Alkyl Analogues Nucleosides Nucleotides Nucleic Acids 1999, 18, 1883-1903; and Priego et al. Recent Advances in Thymidine Kinase 2 (TK2) Inhibitors and New Perspectives for Potential Applications. Current Pharmaceutical Design, 2012, 18, 2981-2994

FIG. 26 presents a non-limiting example of ligands that bind to Potassium Voltage-Gated Channel Subfamily Q Member 1 (KCNQ1). For additional non-limiting examples, see Mattmann Identification of (R)—N-(4-(4-methoxyphenyl)thiazol-2-yl)-1-tosylpiperidine-2-carboxamide, ML277, as a novel, potent and selective Kv7.1 (KCNQ1) potassium channel activator. Bioorg Med Chem Lett. 2012 Sep. 15; 22(18): 5936-5941; Salata, J. et al. A Novel Benzodiazapine that Activated Cardiac Slow Delayed Rectifier K+Currents. Molecular Pharmacology. 1998, 53, 220; Abbott, G. KCNQs: Ligand- and Voltage-Gated Potassium Channels. Front. Physiol. 2020, 11, 583.

FIG. 27 presents a non-limiting example of ligands that bind to Stimulator of Interferon Genes (transmembrane protein 173, ERIS, MITA, TMEM173, encoded by gene STING1). For additional non-limiting examples, see Pryde, D. C. et al. The discovery of potent small molecule activators of human STING. Eur J Med Chem 209, 112869 (2021); Ramanjulu, J. M. et al. Design of amidobenzimidazole STING receptor agonists with systemic activity. Nature 564, 439-443 (2018).

FIG. 28 is a non-limiting example of a Formula of the present invention.

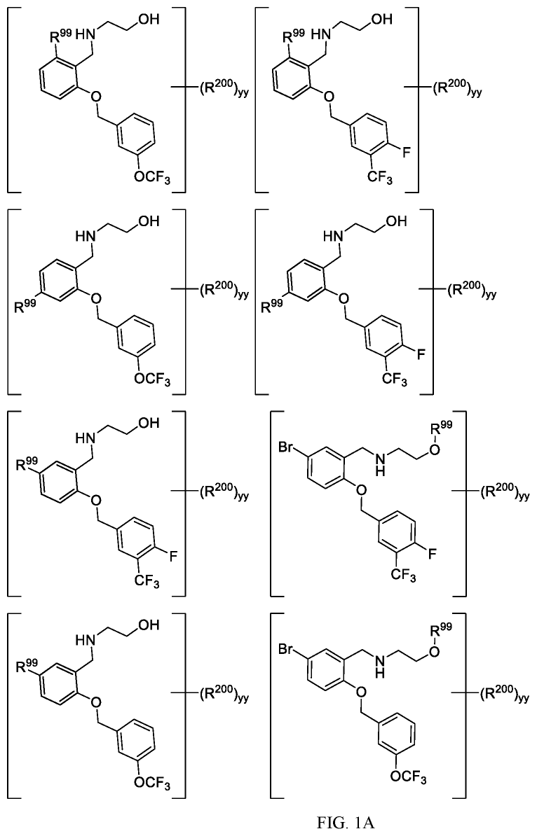

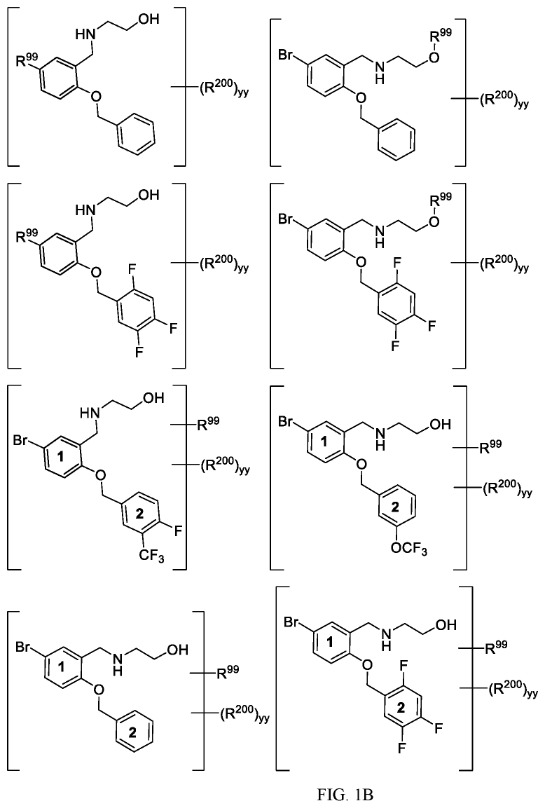

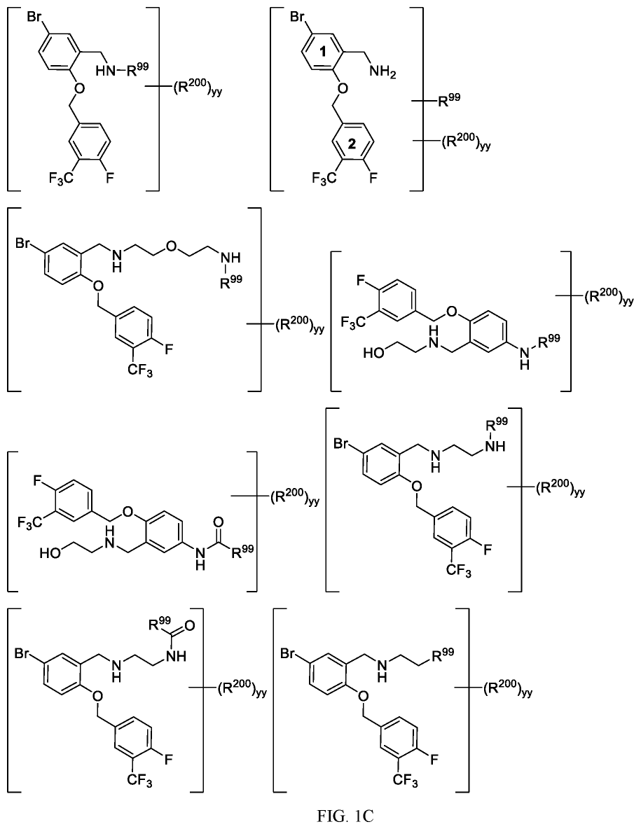

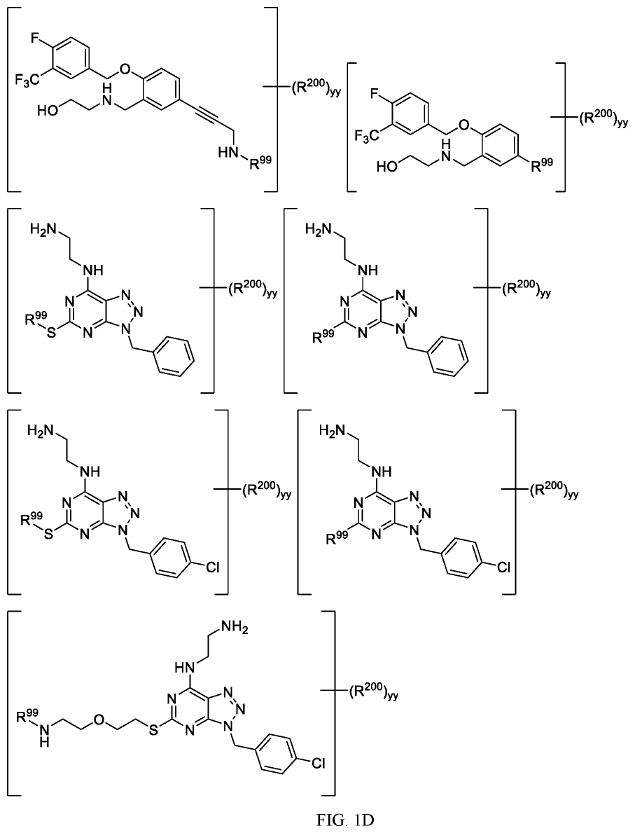

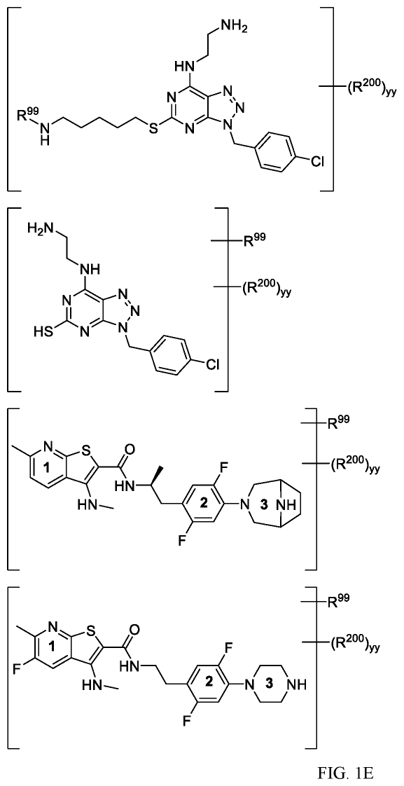

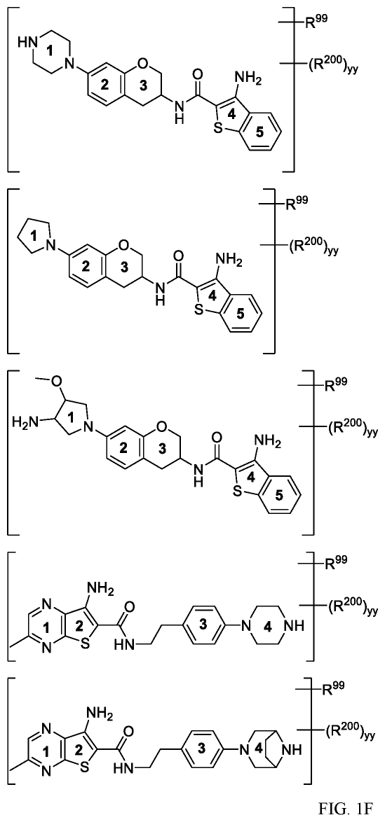

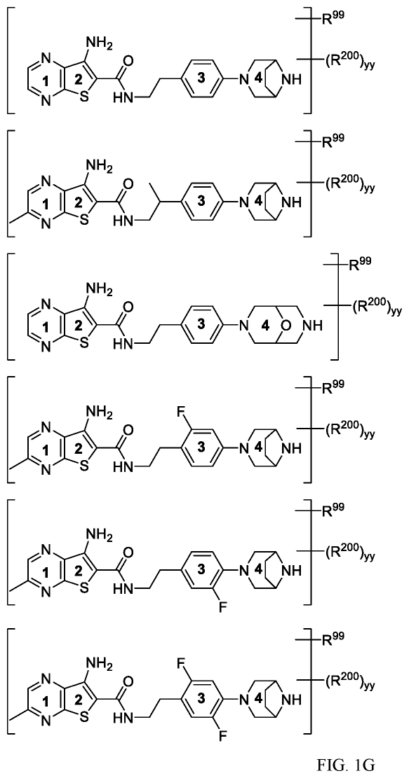

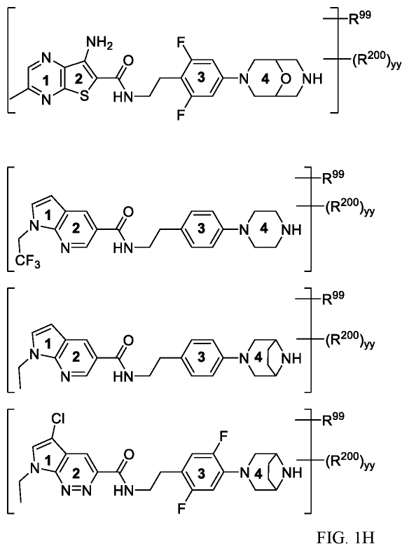

### DETAILED DESCRIPTION OF THE INVENTION

Protein stabilizing and/or function restoring compounds and their uses and manufacture are provided that stabilize a Target Ubiquitinated Protein by deubiquitinating it and in some embodiments restore at least a partial amount of the protein's function. The protein stabilizing and/or function restoring compounds described herein include a USP28 Targeting Ligand, a Ubiquitinated Protein Targeting Ligand, and optionally a Linker. In some embodiments, the protein's function is restored by at least about 1%, 2.5%, 5%, 7.5%, 10%, 15% or more over the native protein or a mutated or altered form of the protein, as relevant in context.

When a deubiquitinase removes ubiquitins from a protein the proteasomal degradation of the protein may be prevented (i.e. the protein is stabilized), the protein may resume its activity (i.e. the protein's function is restored), or the deubiquitination may be insufficient to prevent degradation or restore function. A compound described herein removes ubiquitin from the Target Ubiquitinated Protein in a manner that stabilizes the protein and in some embodiments restore the protein's function (for example restoring at least about 1%, 2.5%, 5%, 7.5%, 10%, 15%, 20%, 25%, 30%, 40%, 50%, 60%, 70%, 80%, 90%, or 100% protein function). By both stabilizing and restoring the protein's function various disorders that are caused by a deficiency of a protein's activity can be treated. For example, disorders caused by loss of function protein mutations or haploid insufficiency can be treated by restoring the function of the downregulated wildtype protein or interest or a mutant thereof. Difficult to treat cancers can also be treated with a protein stabilizing compound of the present invention. For example, cancers that downregulate tumor suppressors can be treated by restoring the function of the tumor suppressor. A protein stabilizing compound described herein can also prompt an immunological response in the treatment of cancer and thus treat the cancer by activating the immune system.

The protein stabilizing compound as described herein in principle embodiments has a stable shelf life for at least 2 months, 3 months, 6 months or 1 year or more neat or as part of a pharmaceutically acceptable dosage form, and itself is pharmaceutically acceptable.

### Embodiments of Formula I

In certain embodiments the compound of the present invention is selected from:

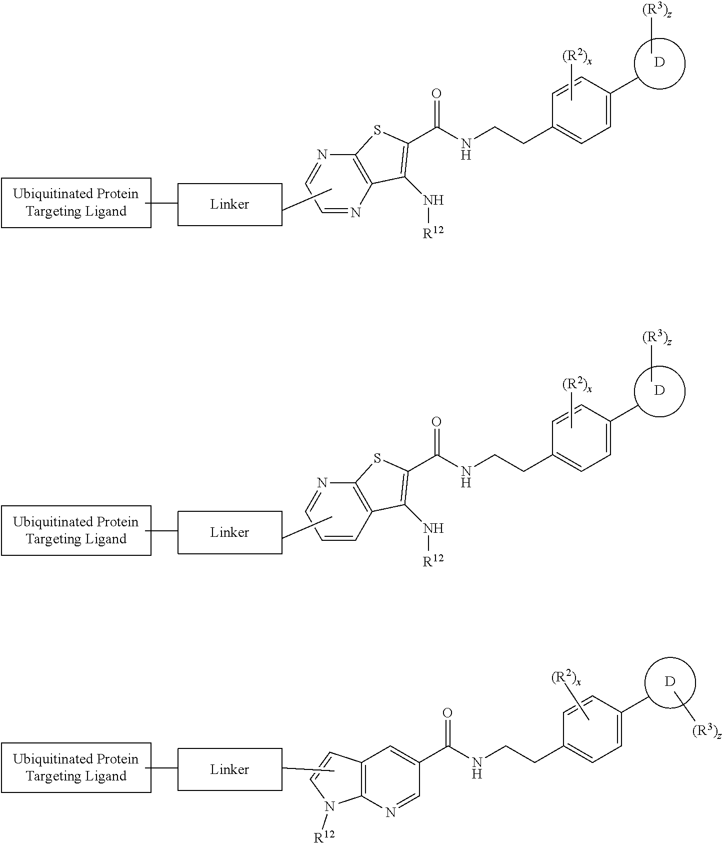

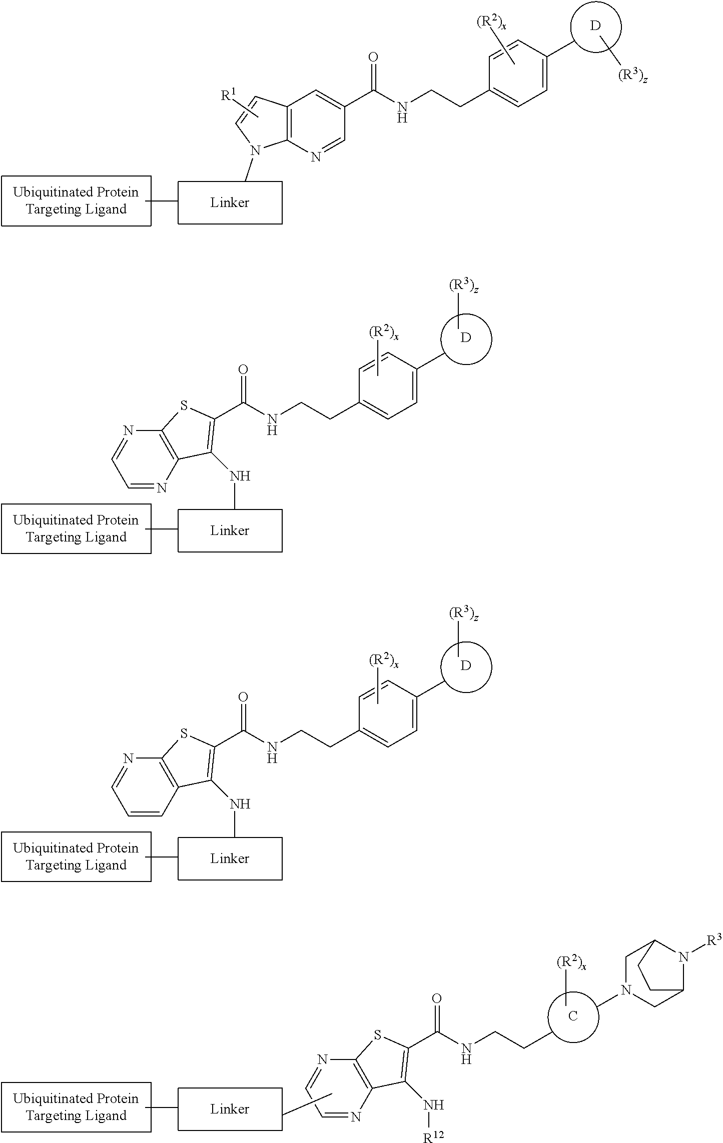

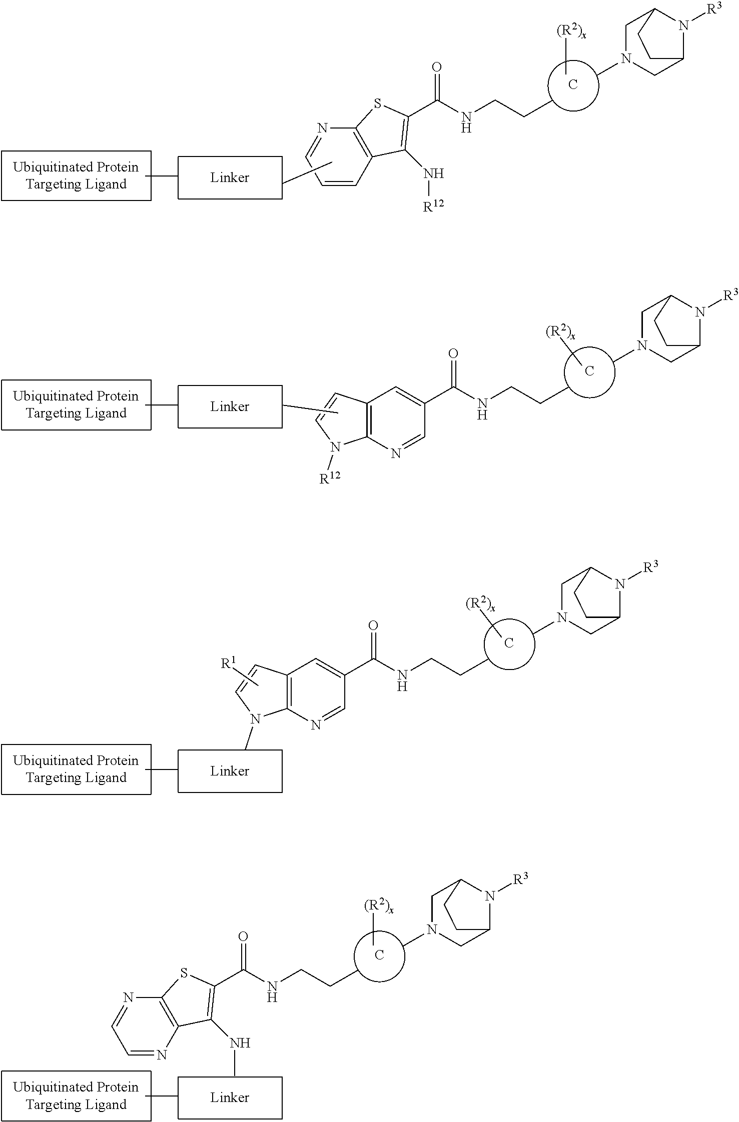

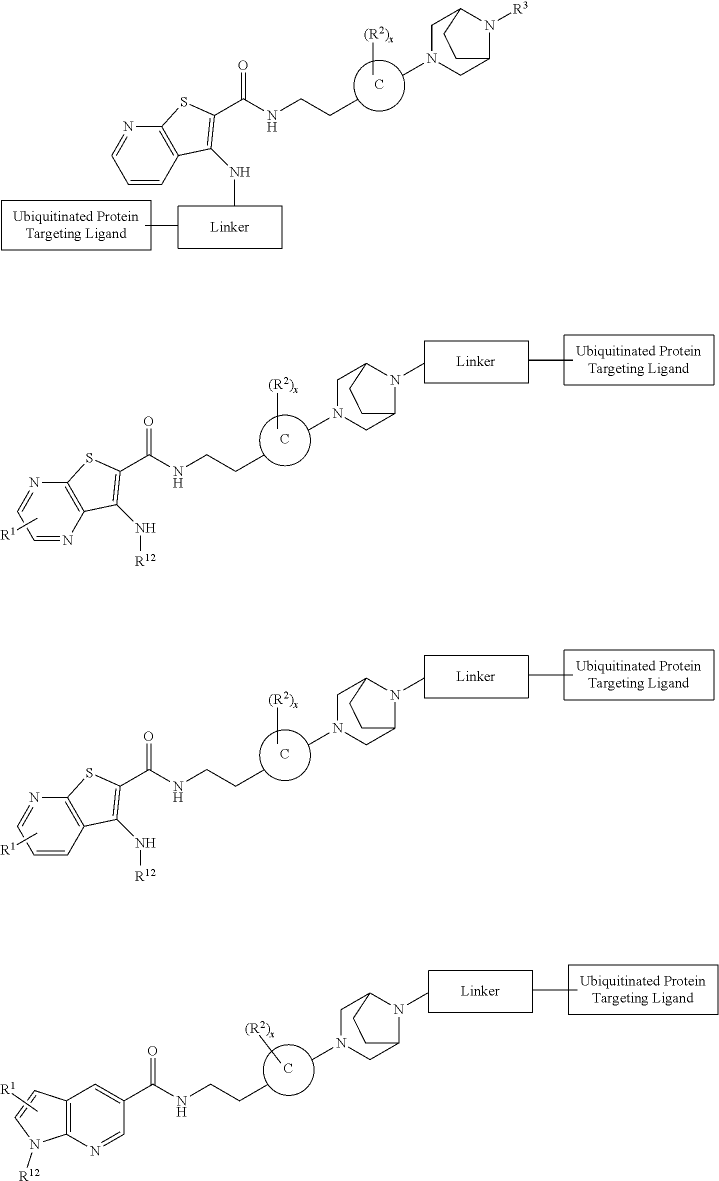

or a pharmaceutically acceptable salt thereof.

In certain embodiments the compound of the present invention is selected from:

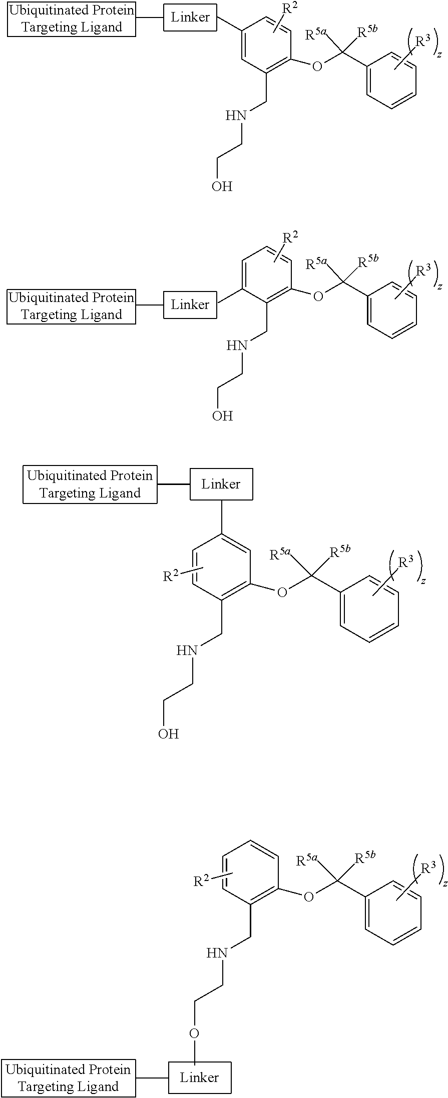

or a pharmaceutically acceptable salt thereof.

In certain embodiments the compound of the present invention is selected from:

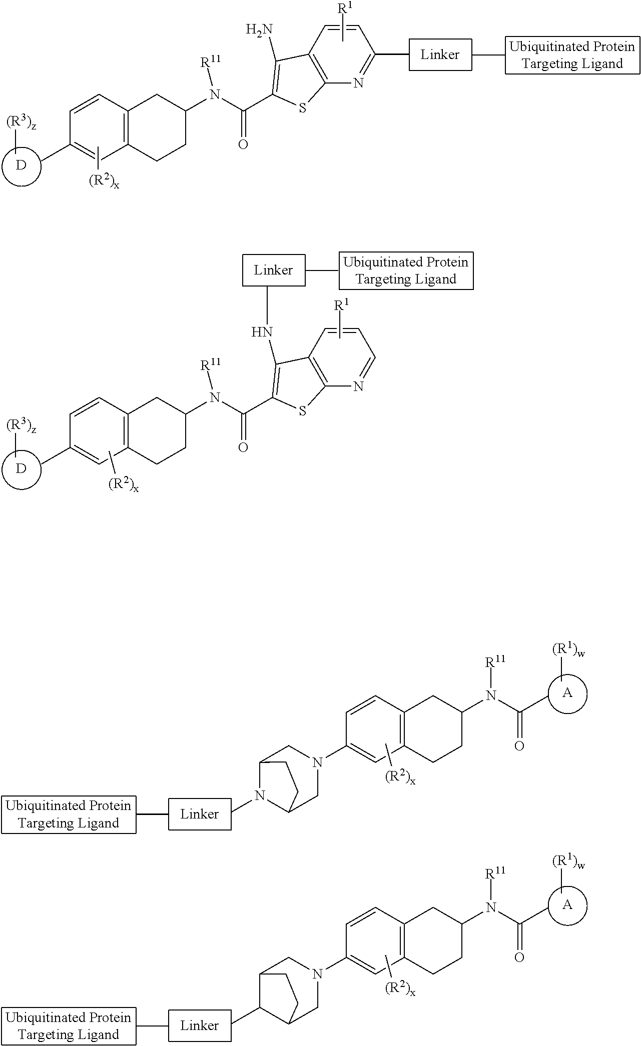

or a pharmaceutically acceptable salt thereof.

In certain embodiments the compound of the present invention is selected from:

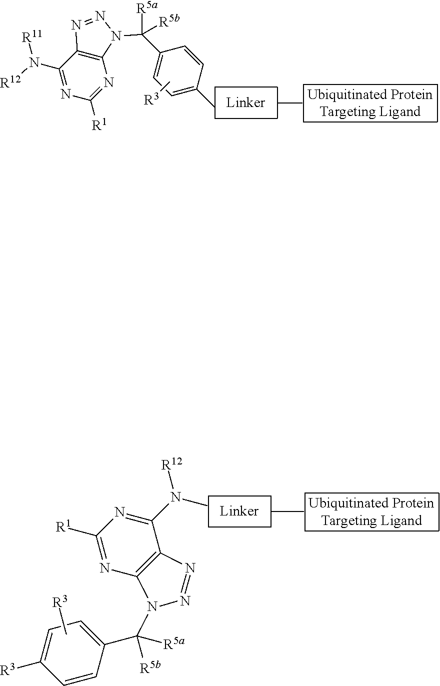

or a pharmaceutically acceptable salt thereof.

In certain embodiments the protein stabilizing compound of the present invention is selected from

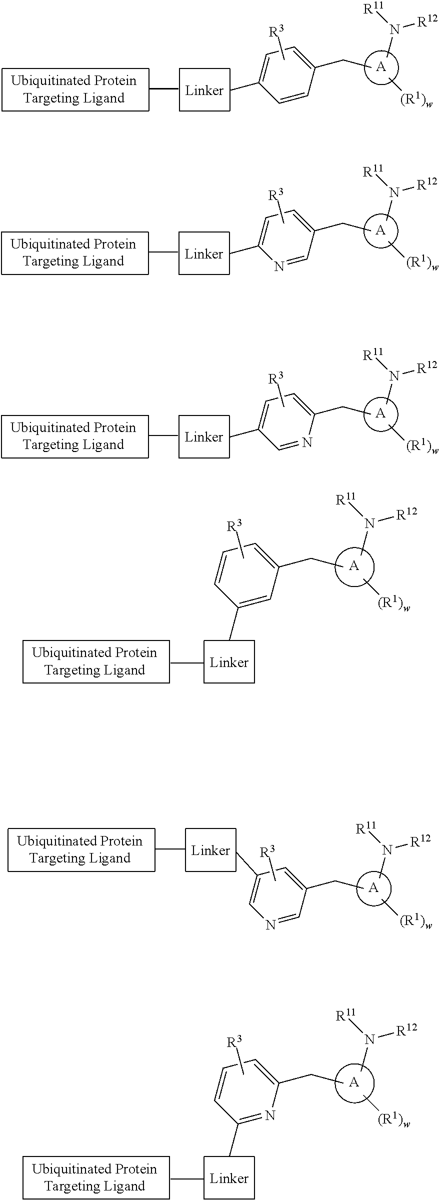

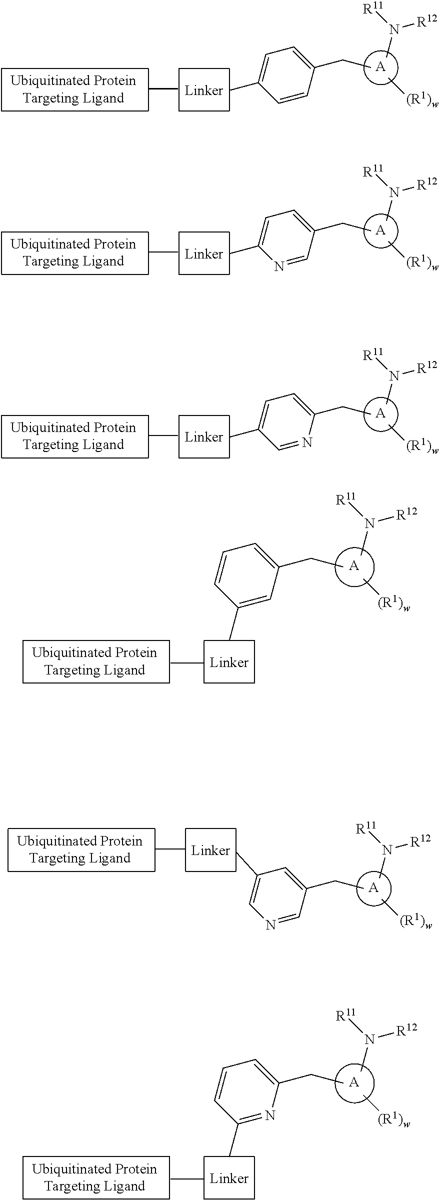

or a pharmaceutically acceptable salt thereof.

In certain embodiments the protein stabilizing compound of the present invention is selected from

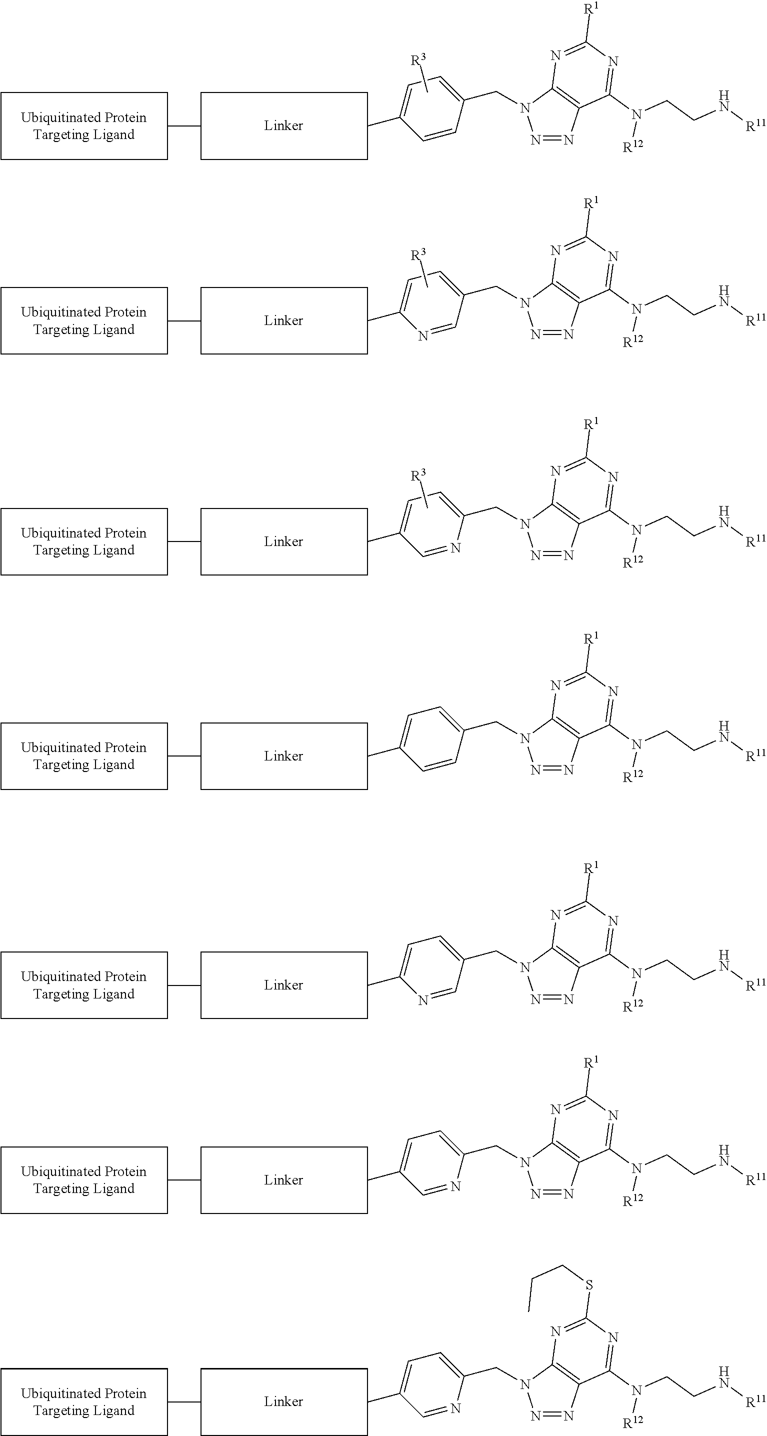

or a pharmaceutically acceptable salt thereof.

In certain embodiments the protein stabilizing compound of the present invention is selected from

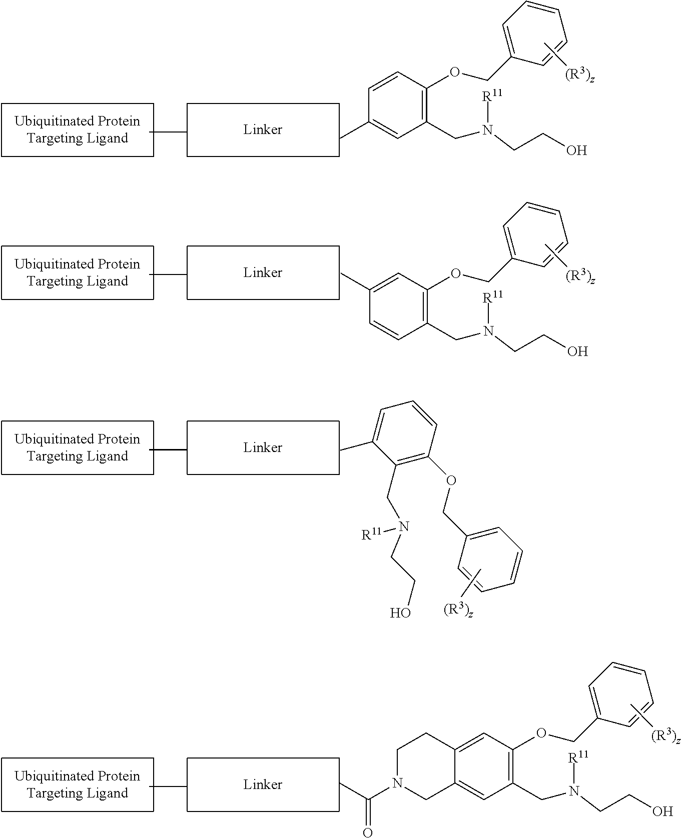

or a pharmaceutically acceptable salt thereof.

In the below embodiments the bond to linker can be on an atom allowed by valence or replace a drawn substituent. For example

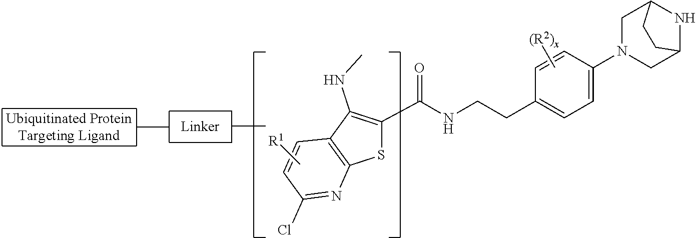

includes:

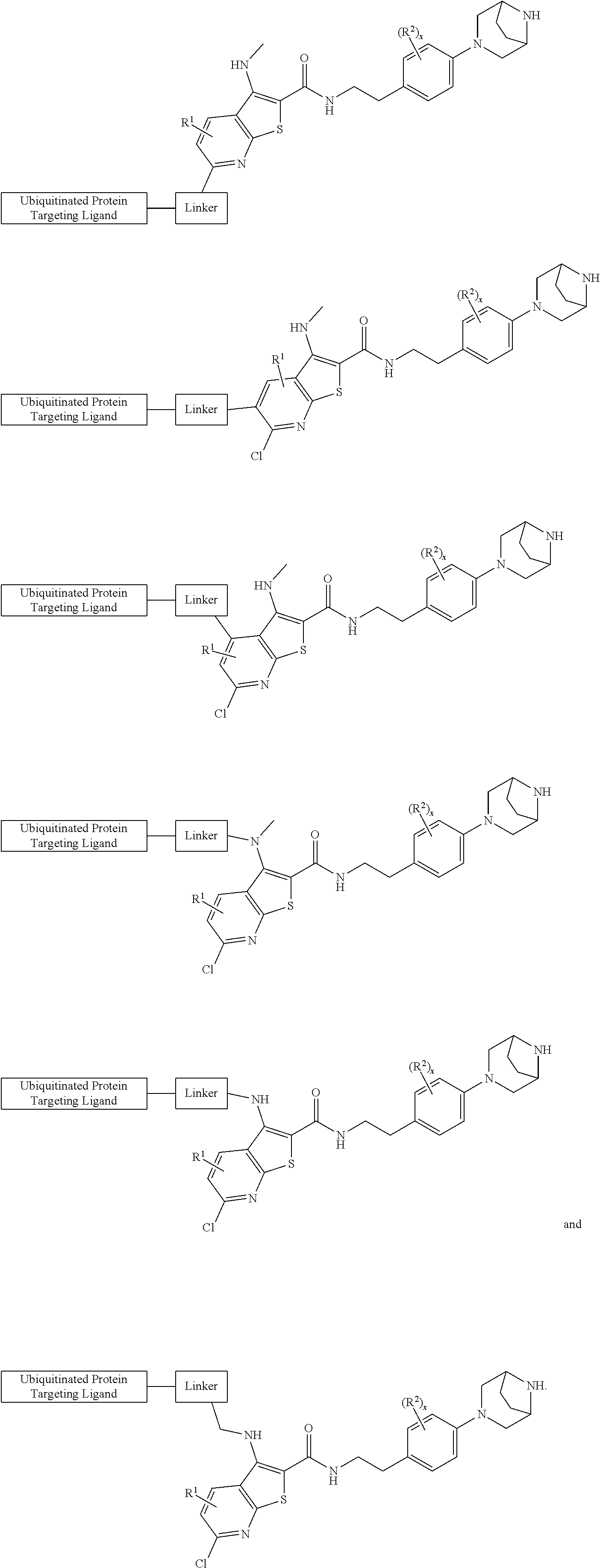

In certain embodiments the protein stabilizing compound of the present invention is selected from:

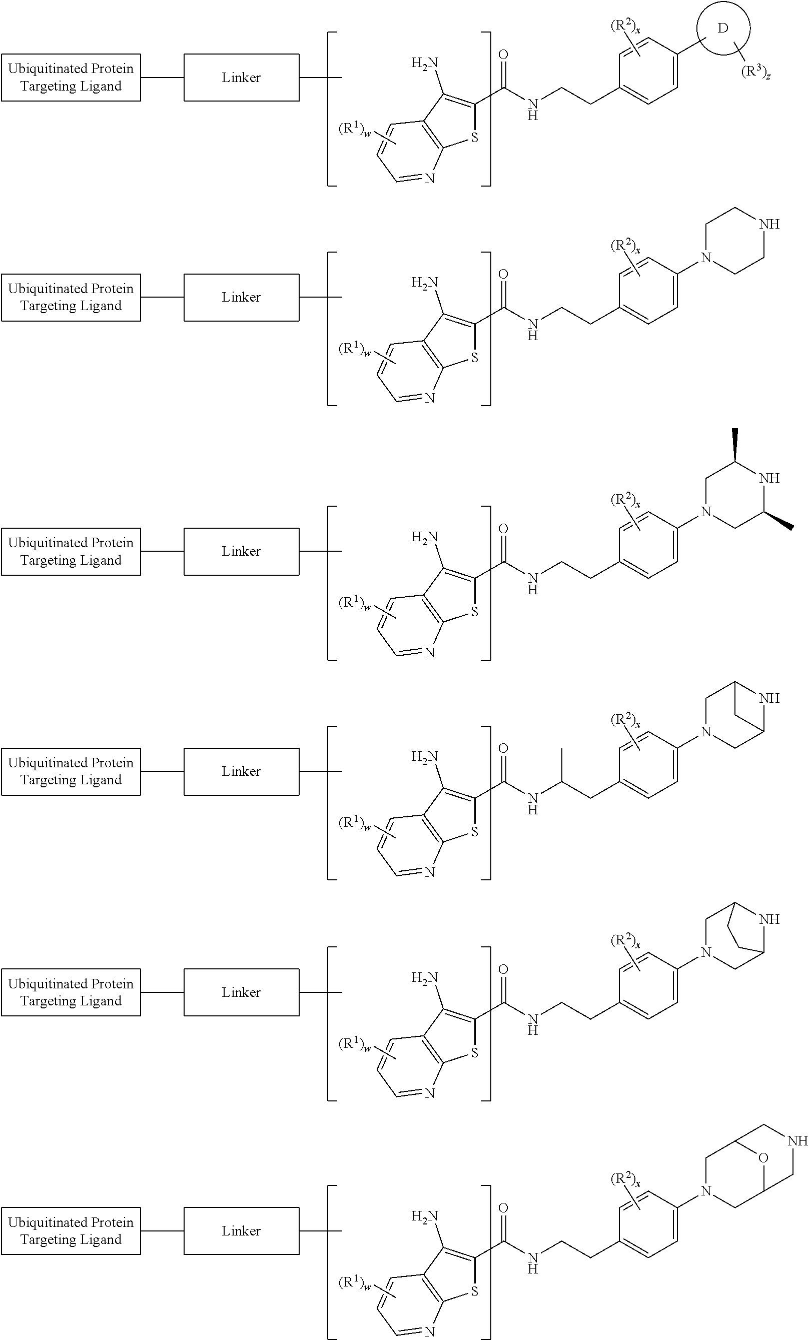

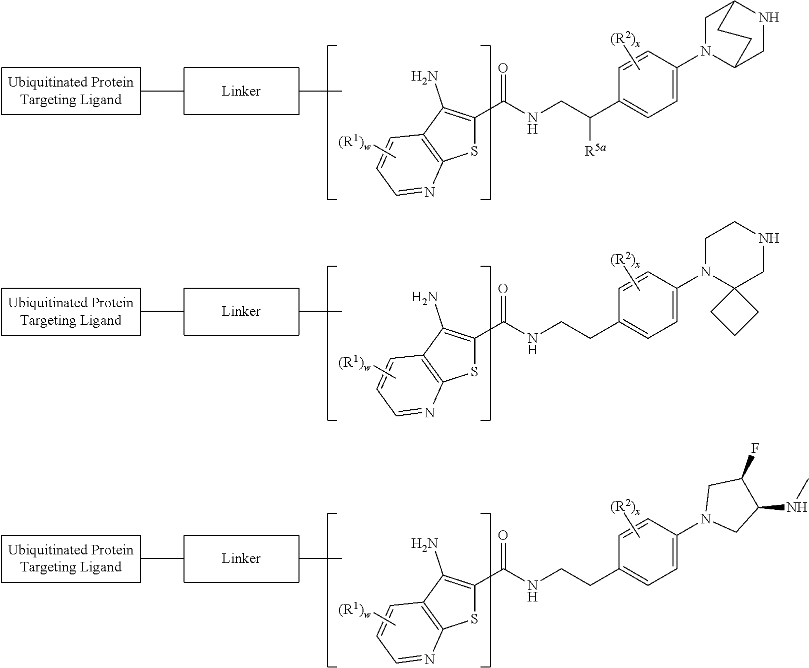

or a pharmaceutically acceptable salt thereof.

In certain embodiments the protein stabilizing compound of the present invention is selected from

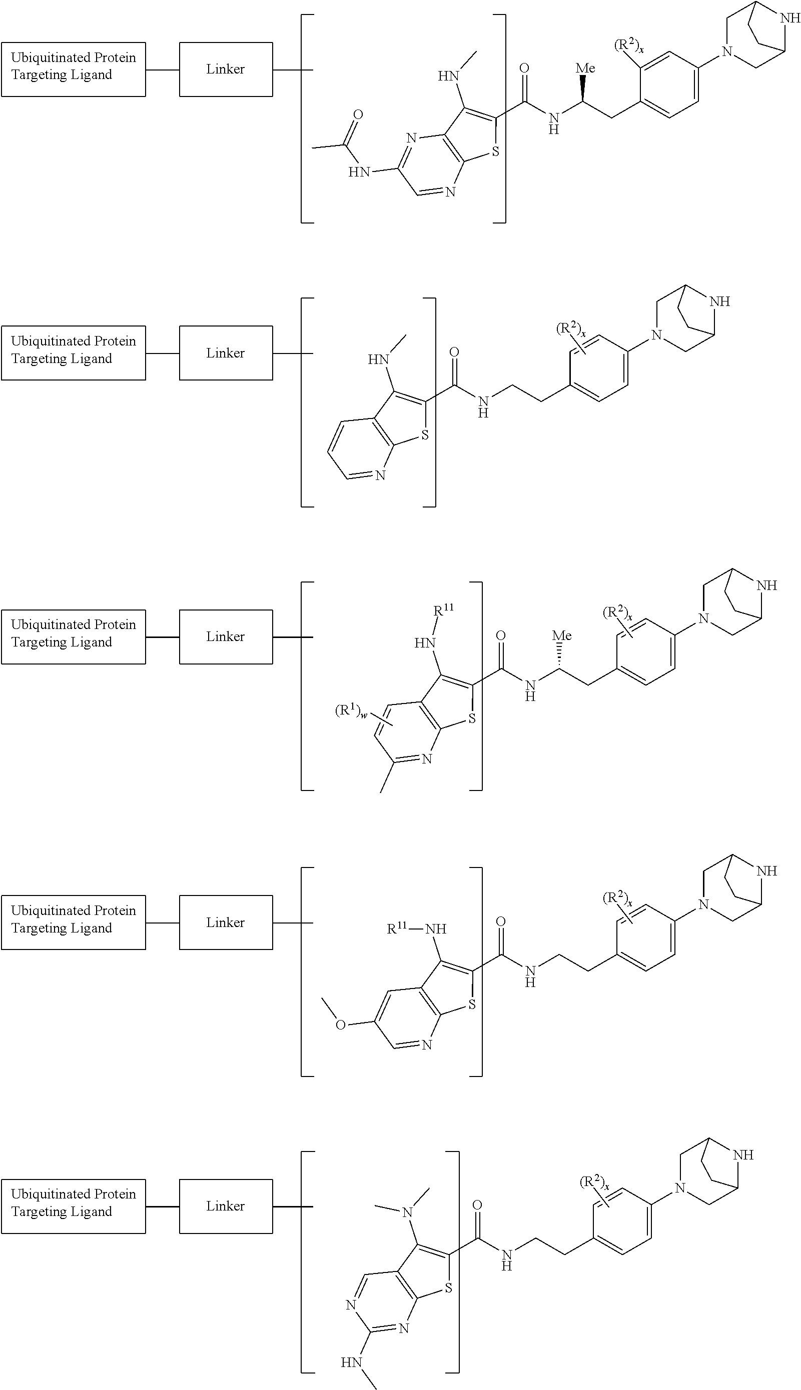

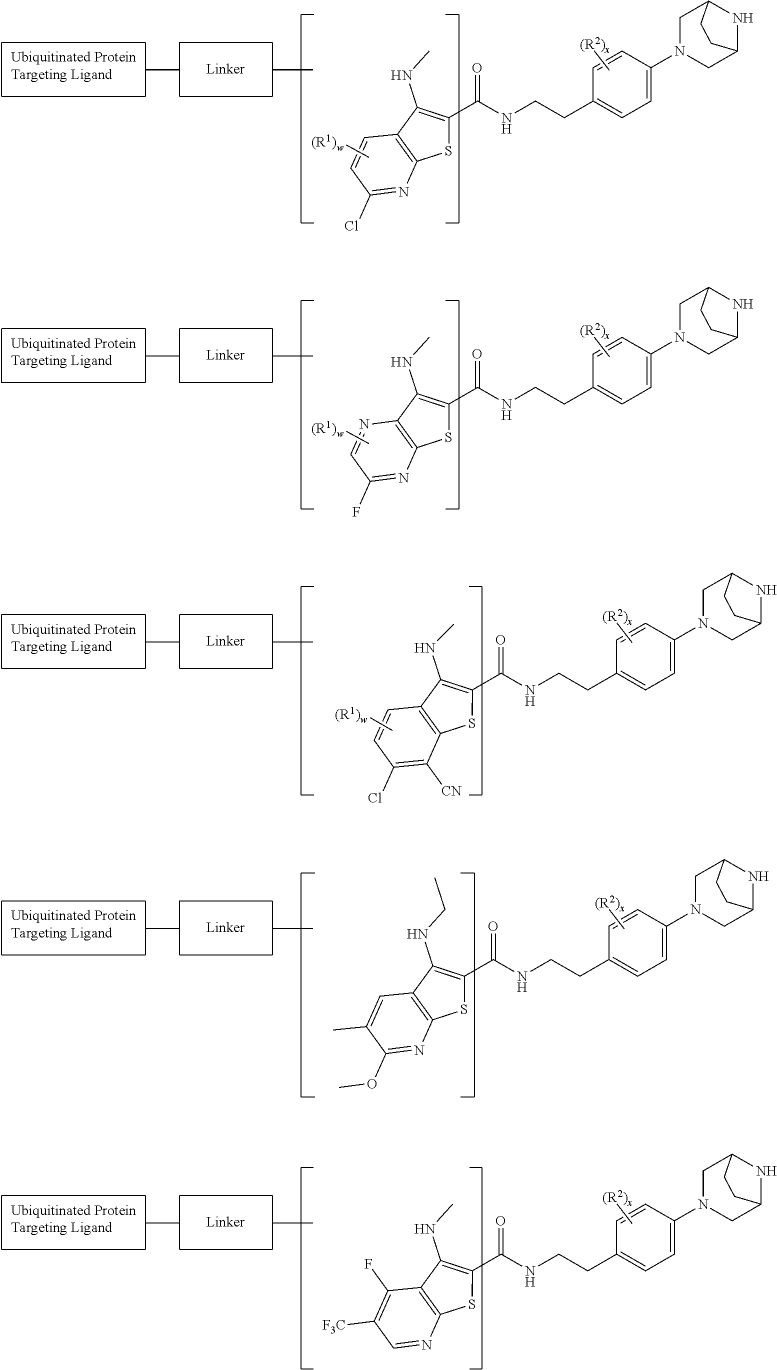

or a pharmaceutically acceptable salt thereof.

In certain embodiments the protein stabilizing compound of the present invention is selected from

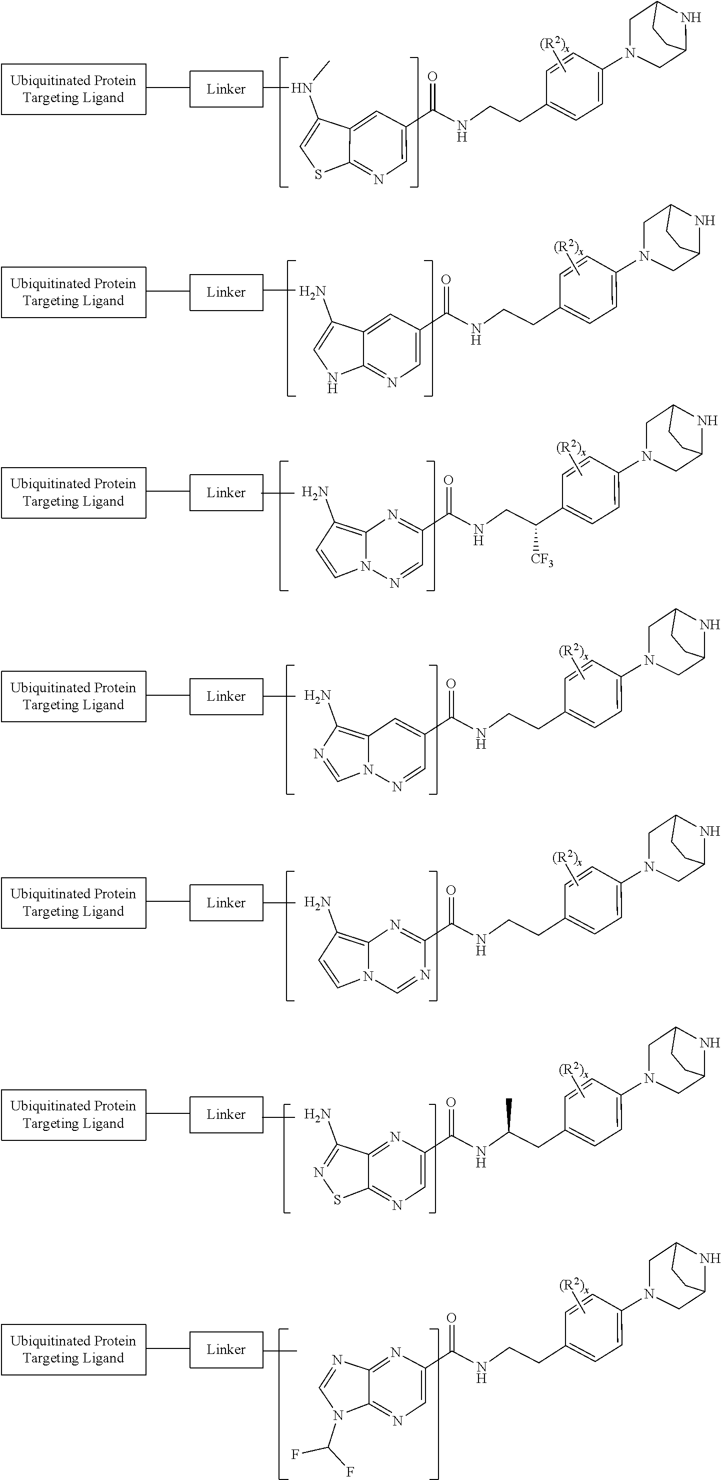

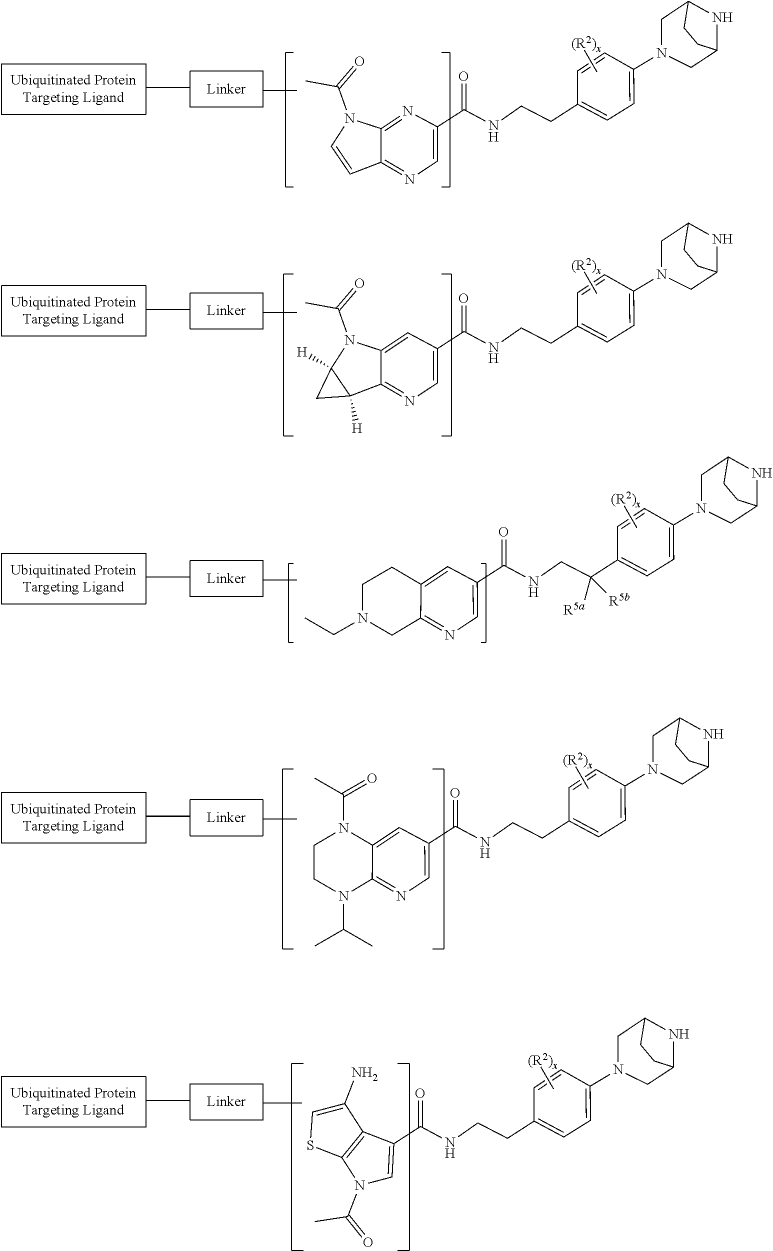

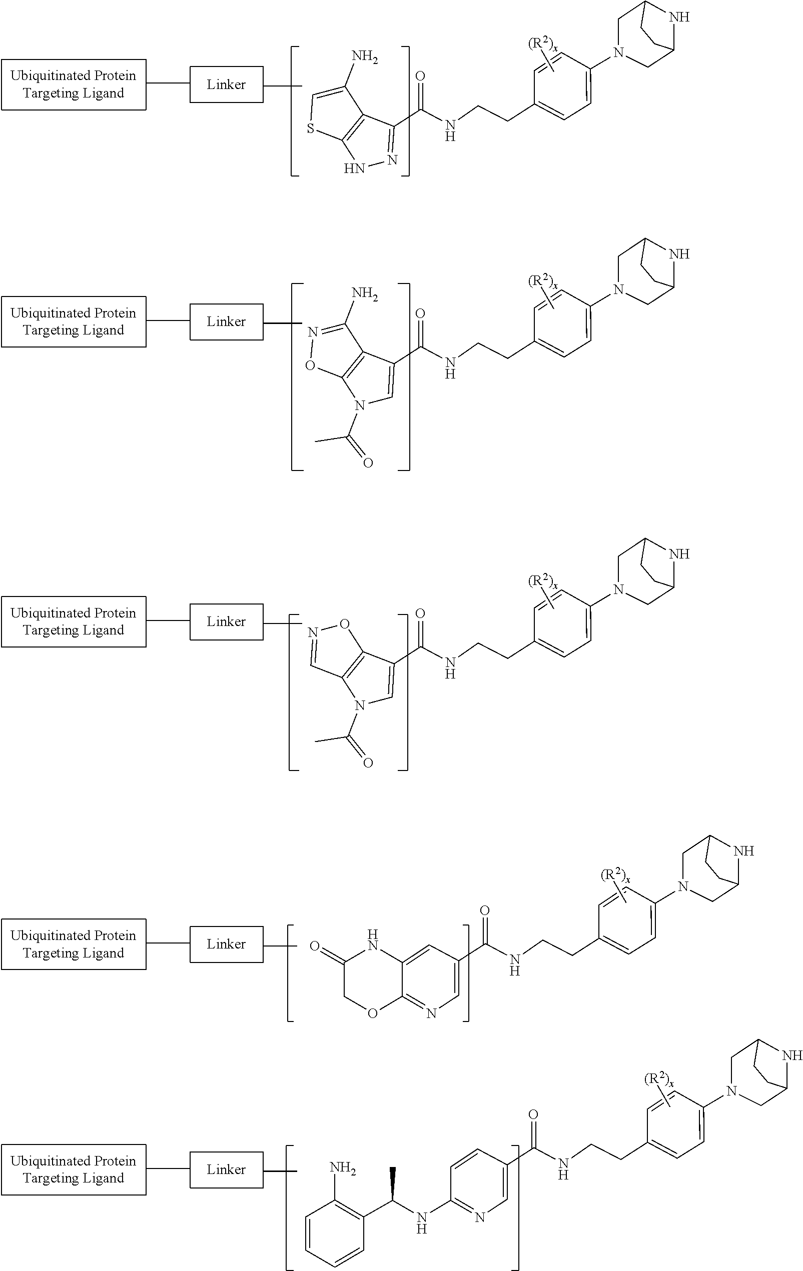

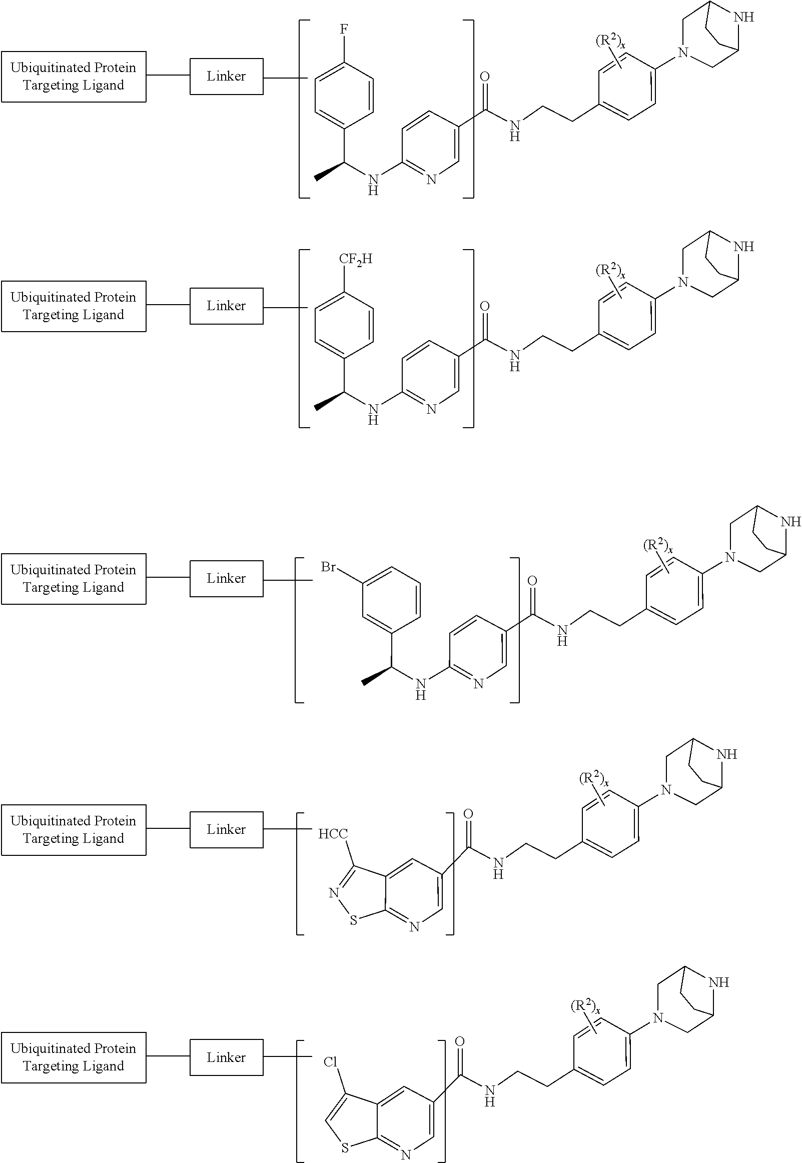

or a pharmaceutically acceptable salt thereof.

In certain embodiments the protein stabilizing compound of the present invention is selected from

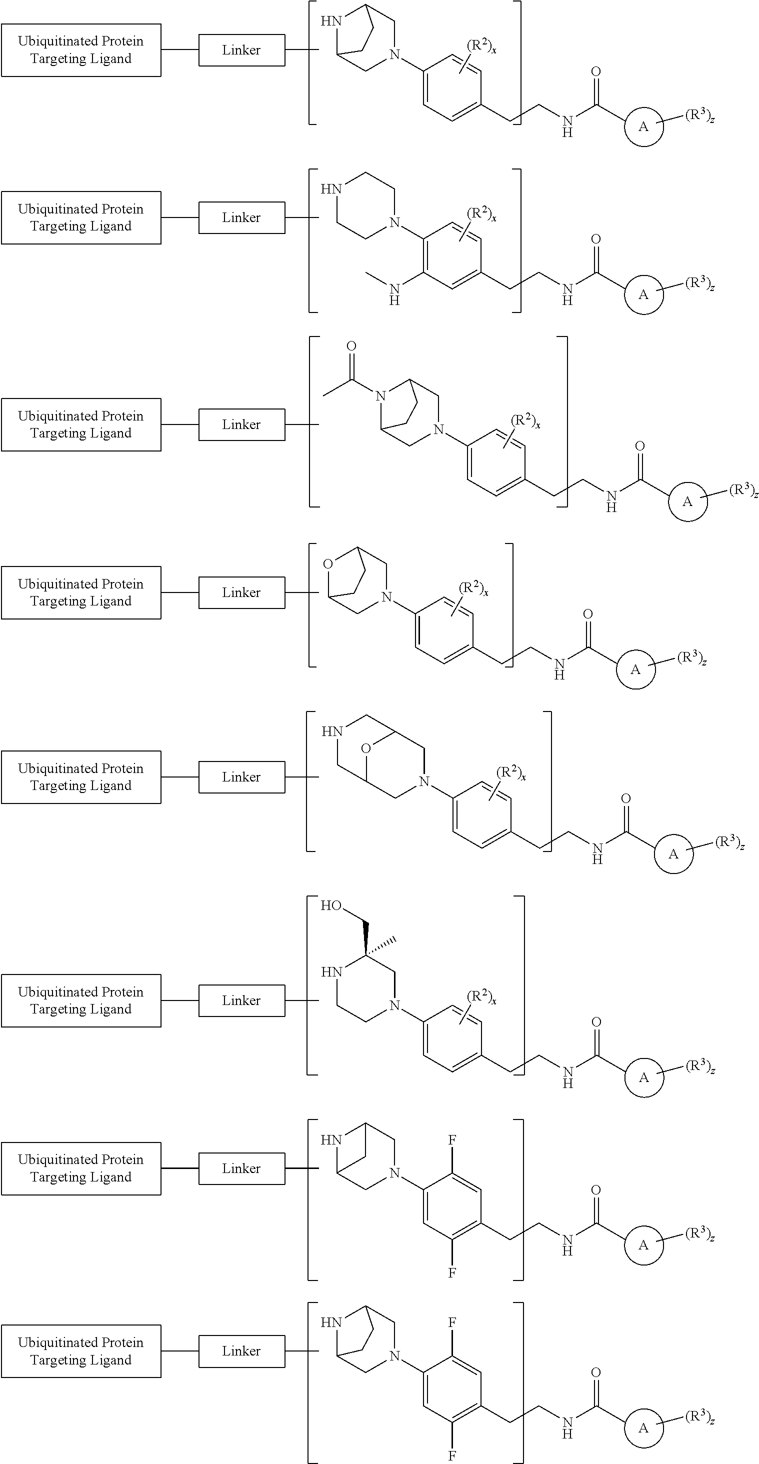

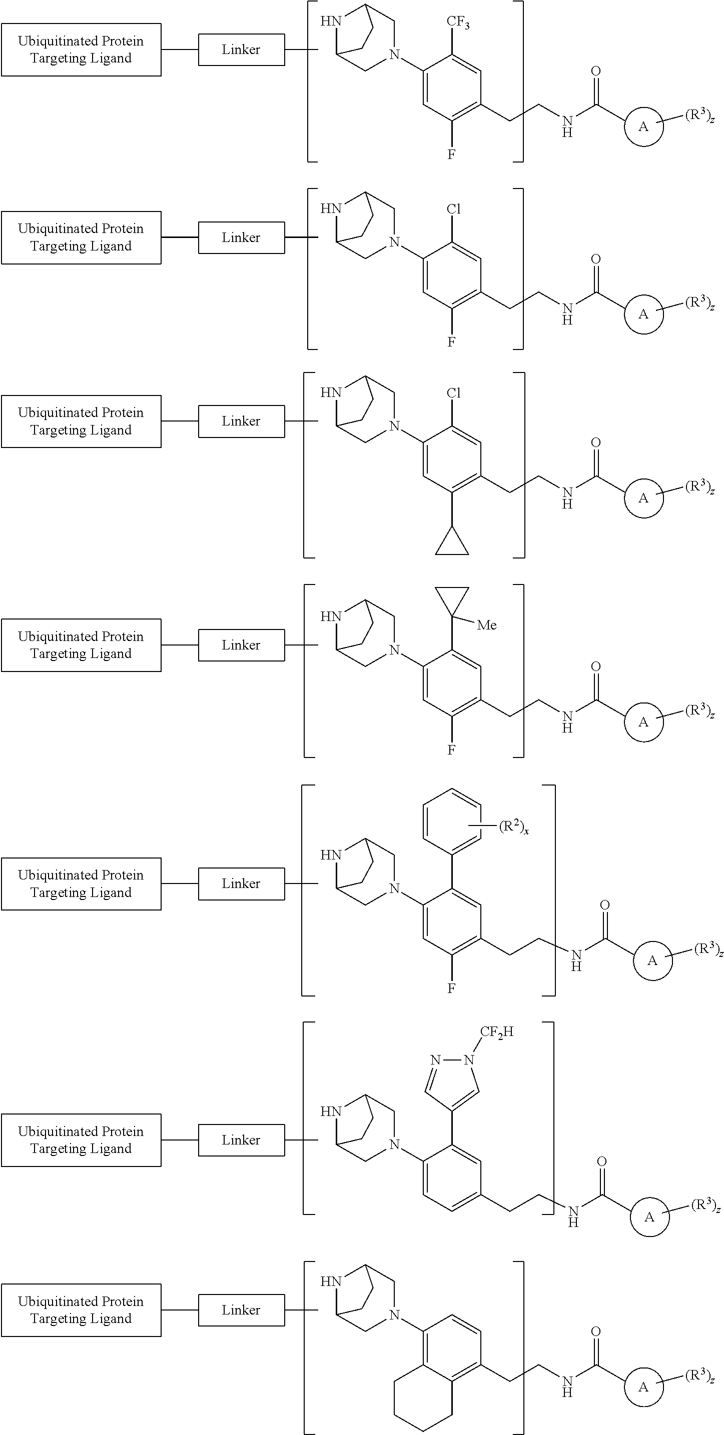

or a pharmaceutically acceptable salt thereof.

In certain embodiments the protein stabilizing compound of the present invention is selected from

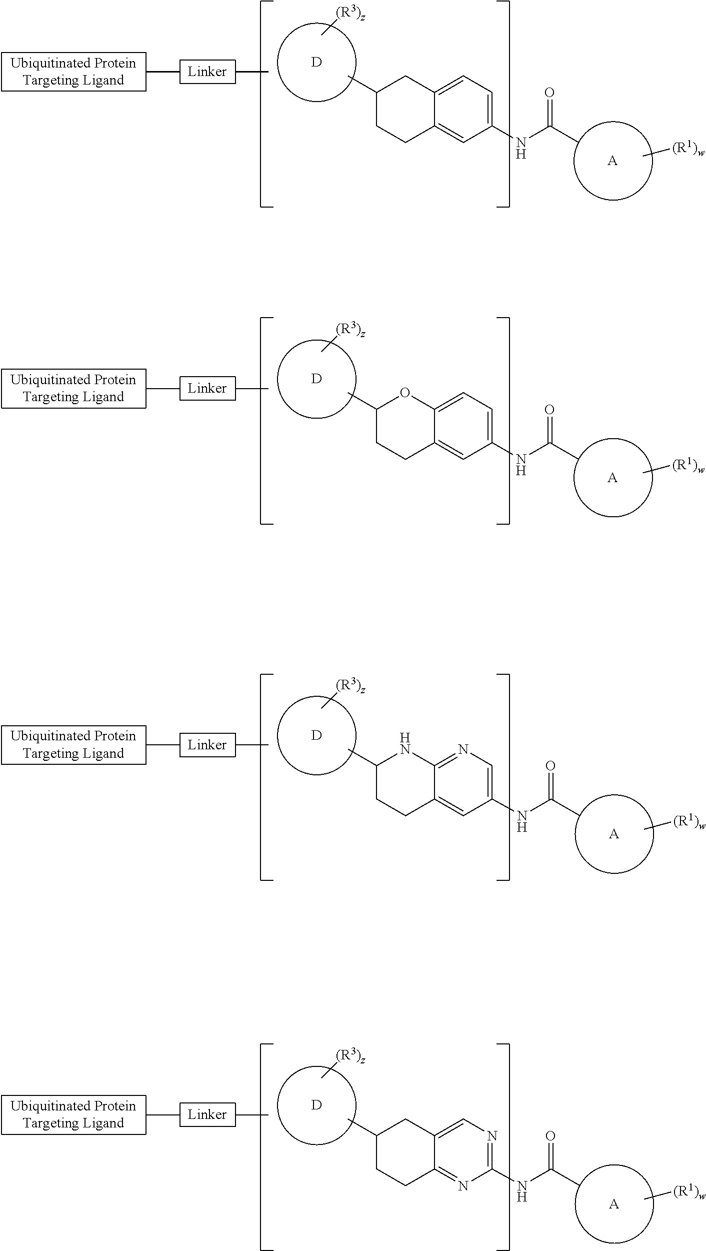

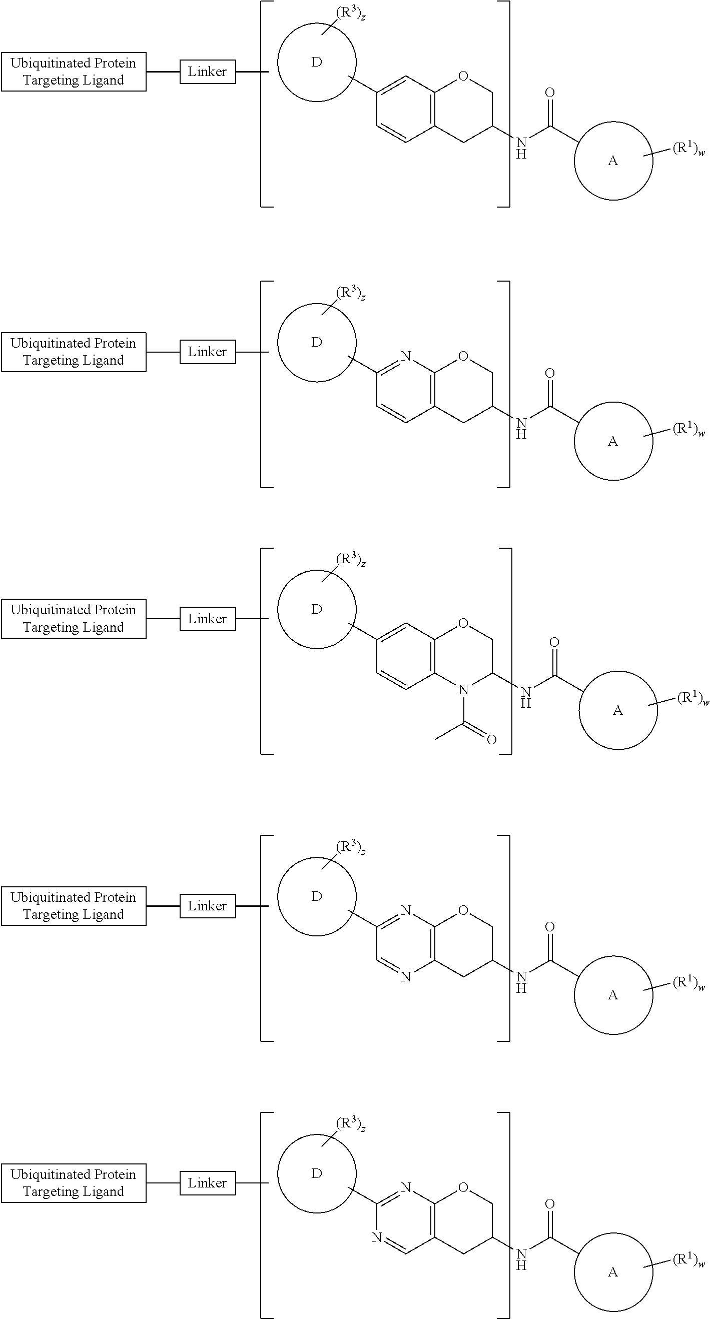

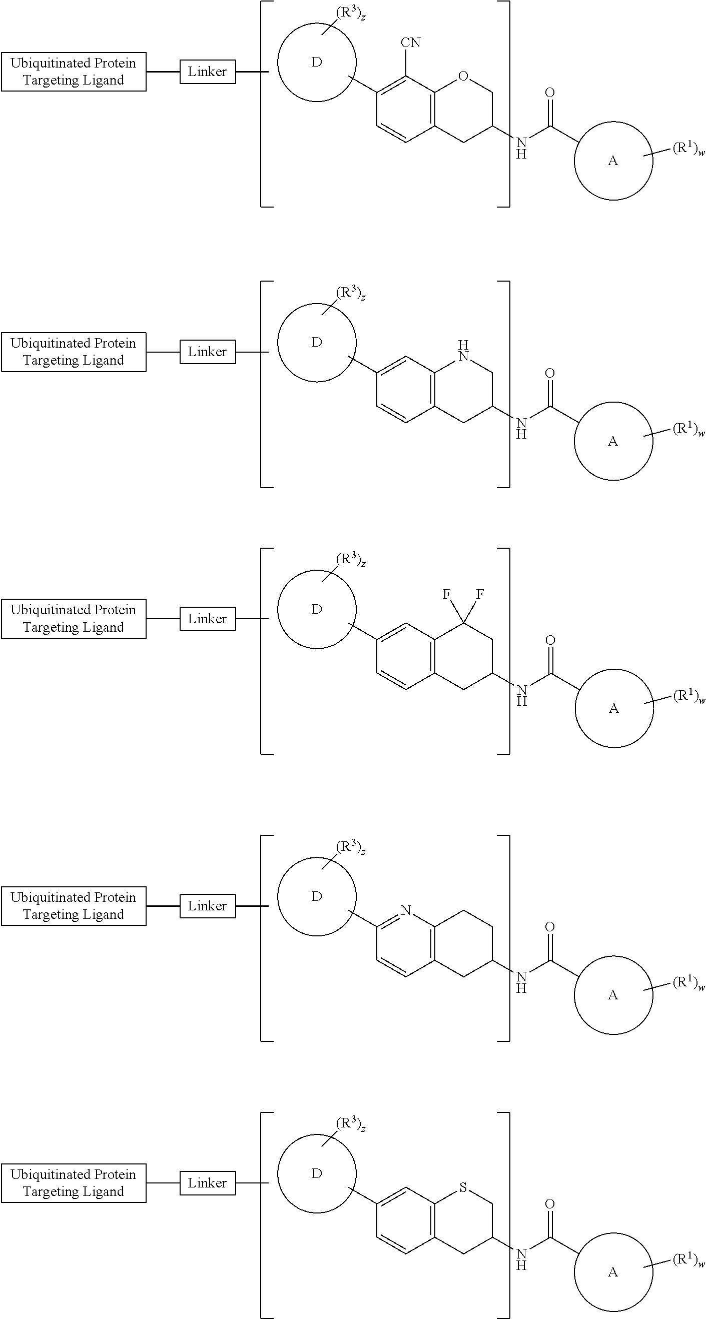

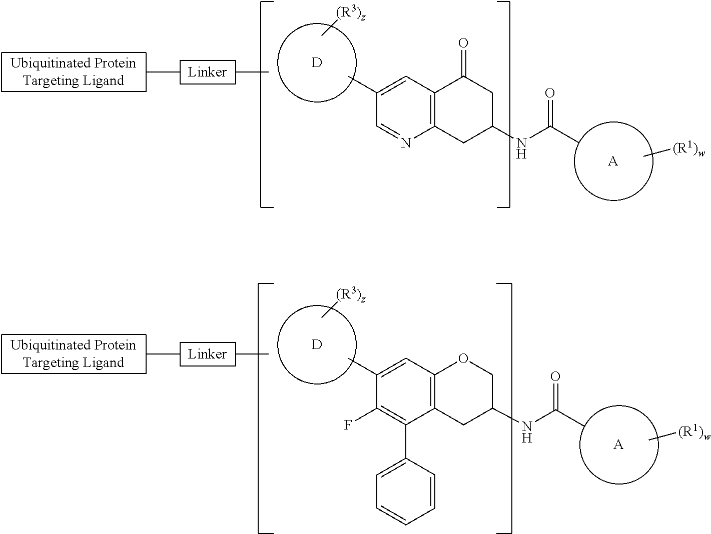

or a pharmaceutically acceptable salt thereof.

### Embodiments of

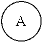

In certain embodiments

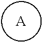

is an aryl group.

In certain embodiments

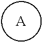

is a phenyl group.

In certain embodiments

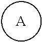

is a heteroaryl group.

In certain embodiments

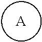

is a bicycle group.

In certain embodiments

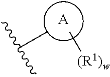

is selected from

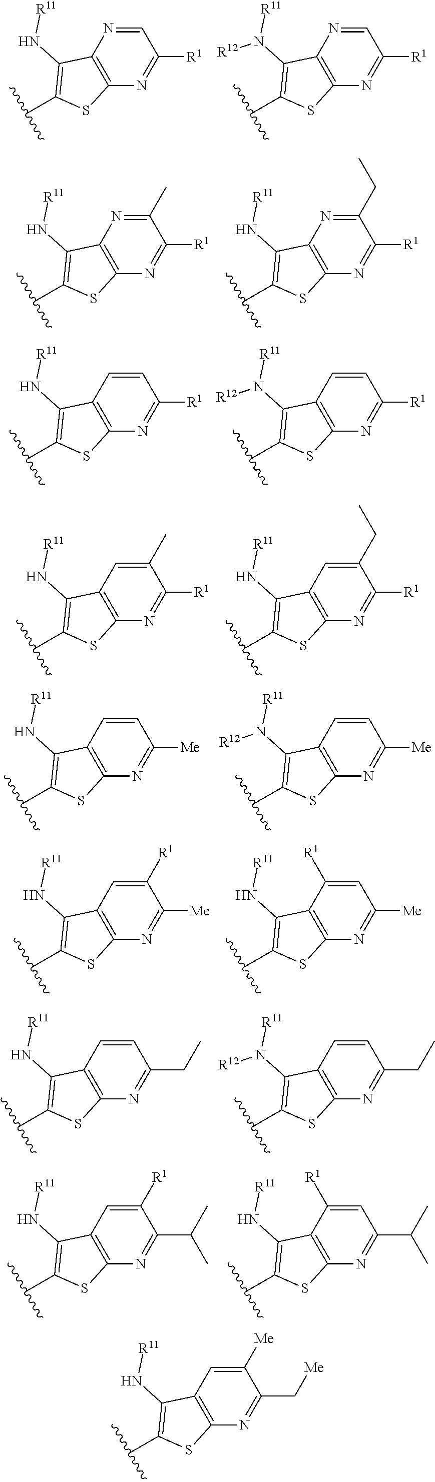

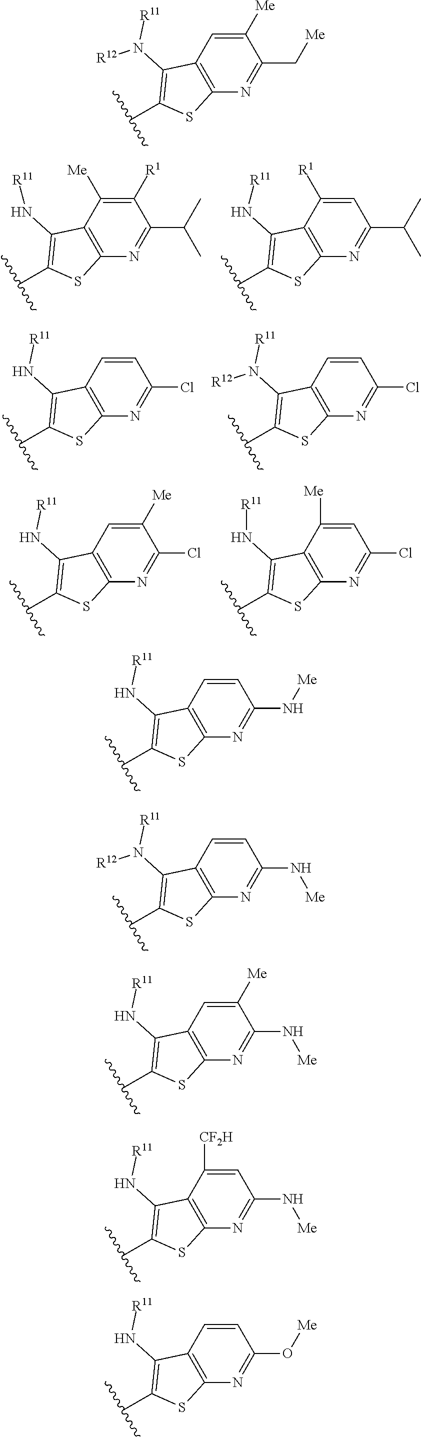

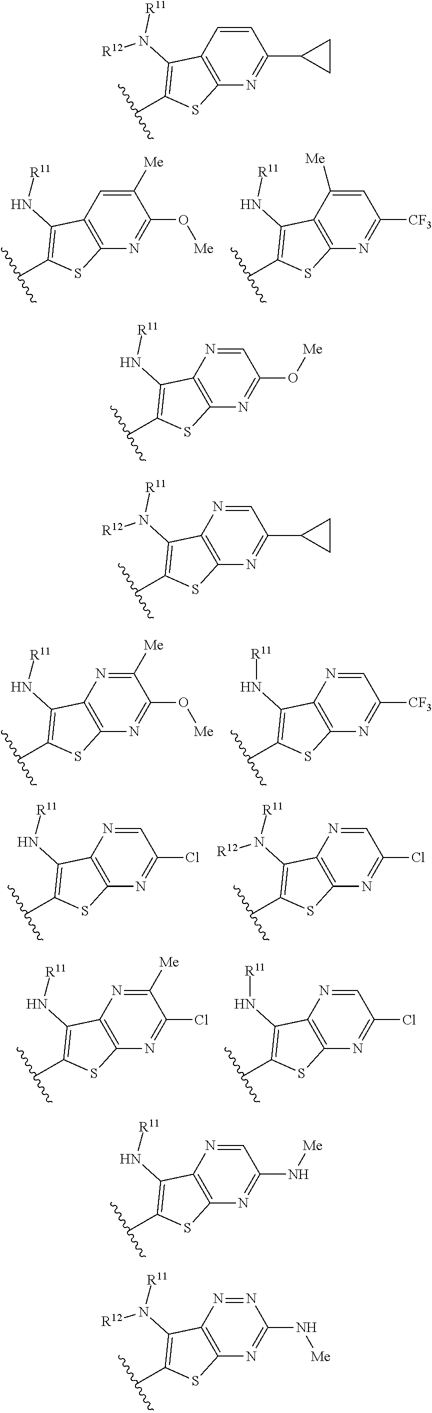

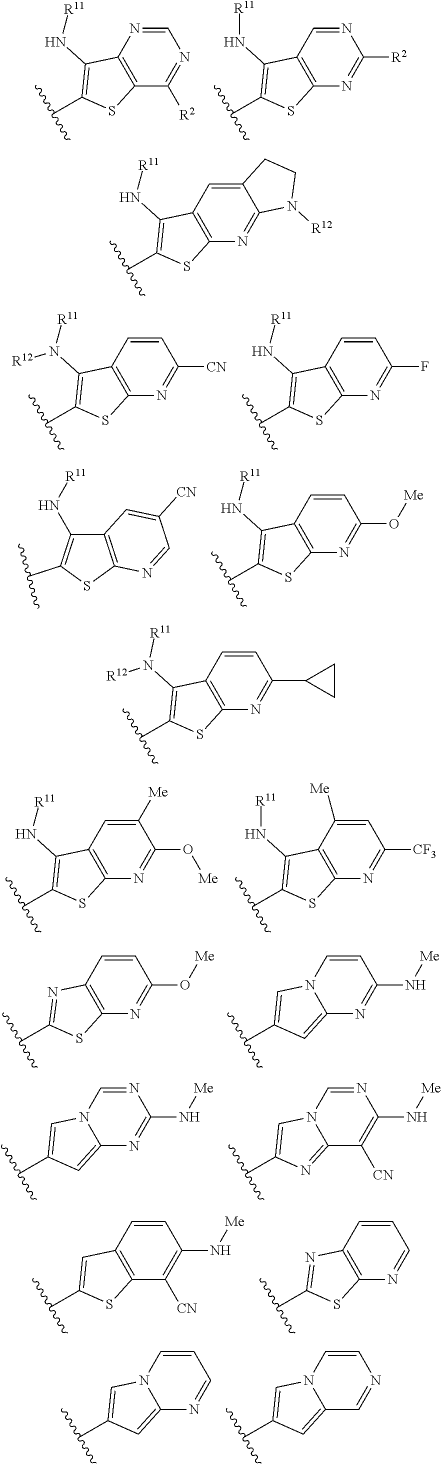

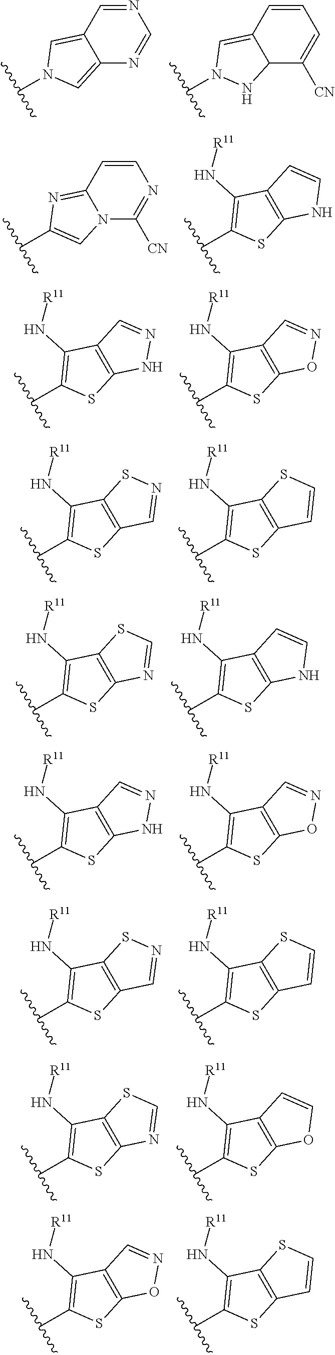

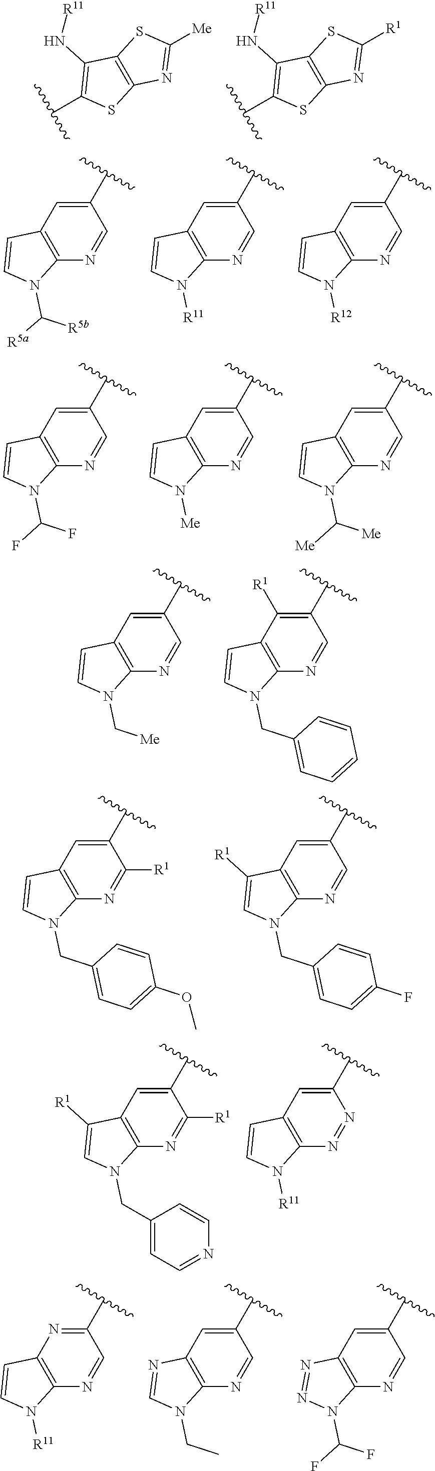

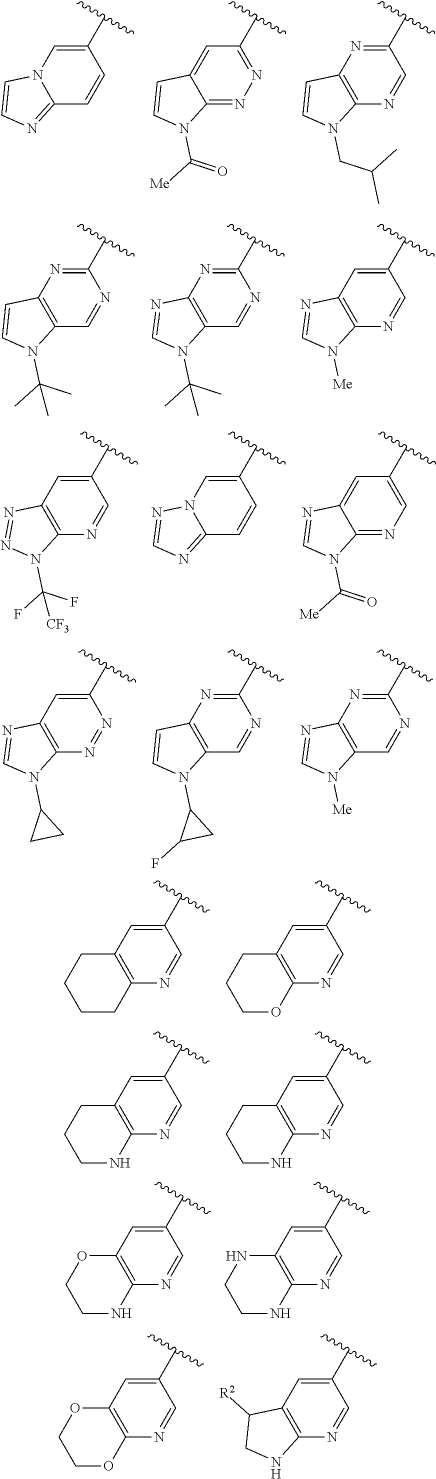

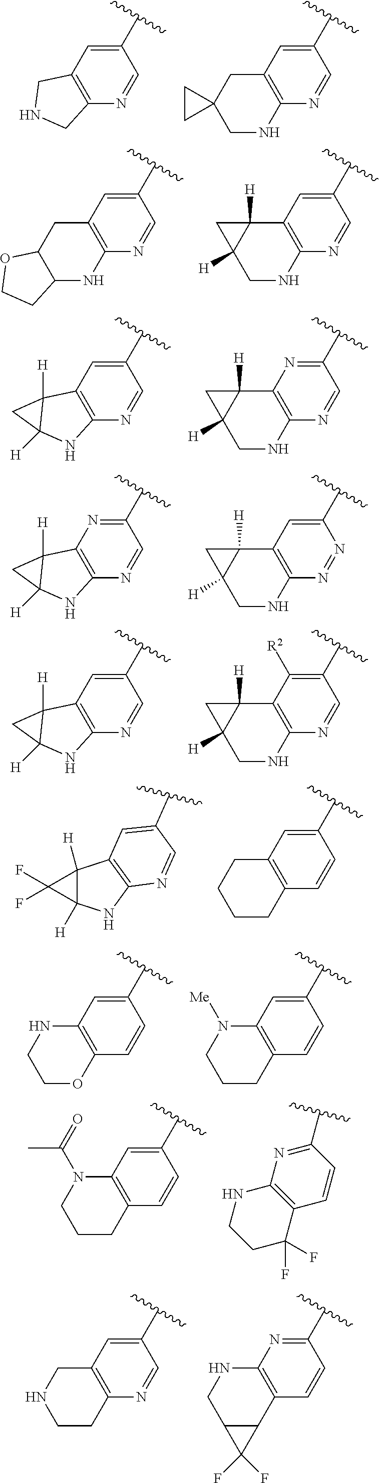

In certain embodiments

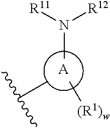

is selected from

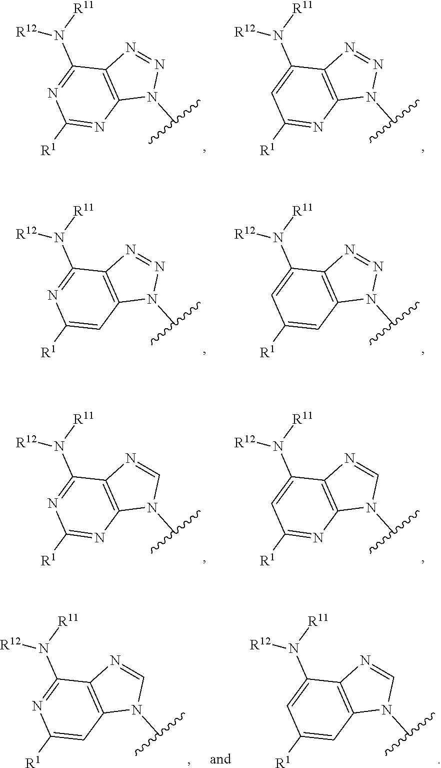

In certain embodiments

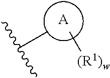

is selected from

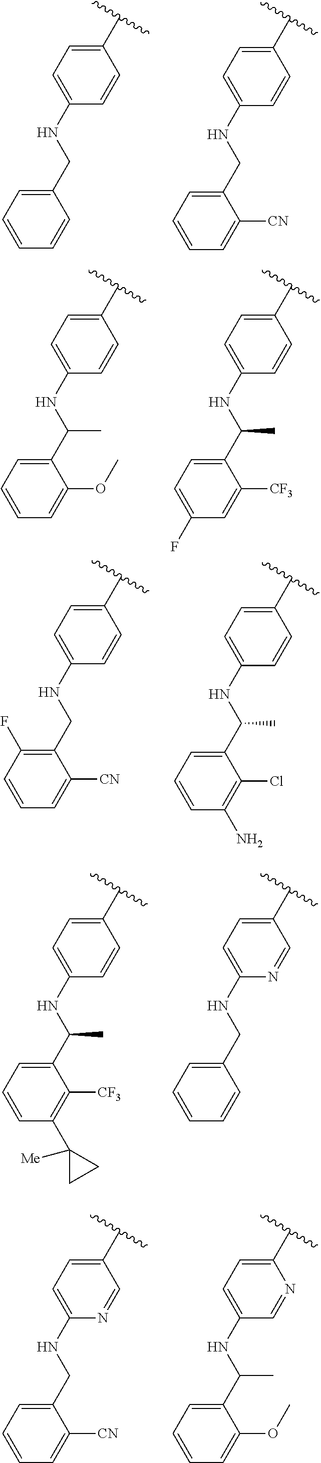

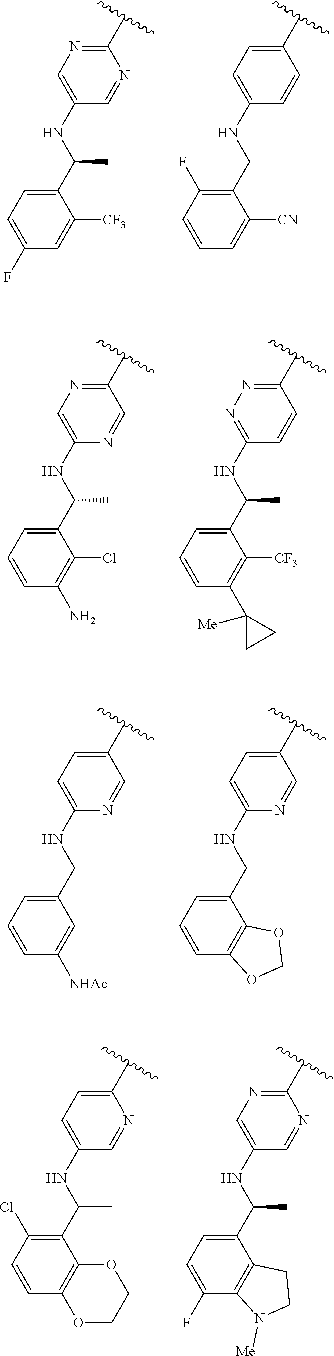

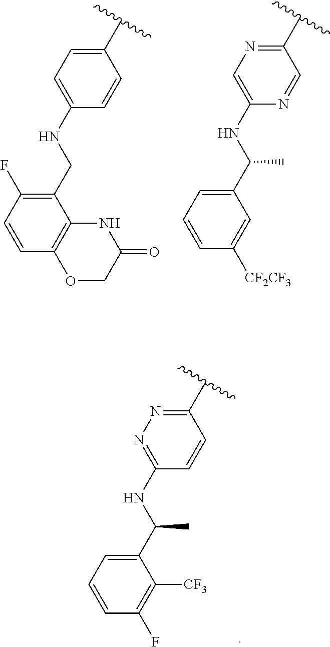

In certain embodiments

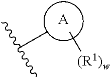

is selected from

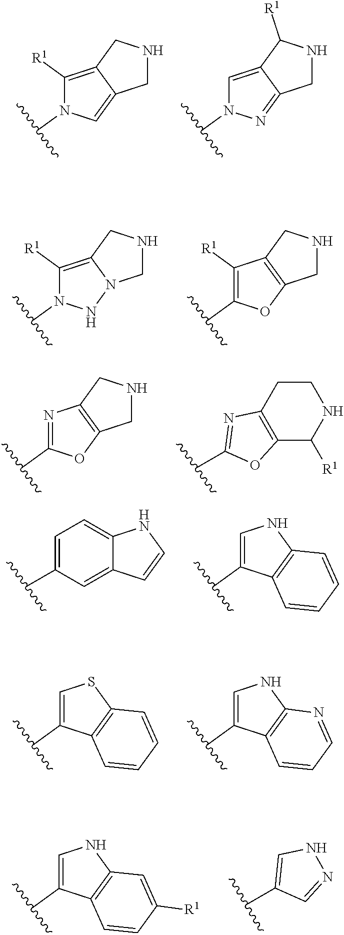

### Embodiments of

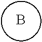

In certain embodiments

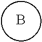

is a bicycle group.

In certain embodiments

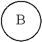

is a bicycle group composed of two aryl rings.

In certain embodiments

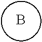

is a bicycle group composed of one heterocyclic and one aryl ring.

In certain embodiments

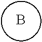

is a bicycle group composed of at least one heterocyclic ring.

In certain embodiments

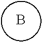

is a bicycle group composed of at least one heteroaryl ring.

In certain embodiments

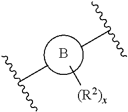

is selected from

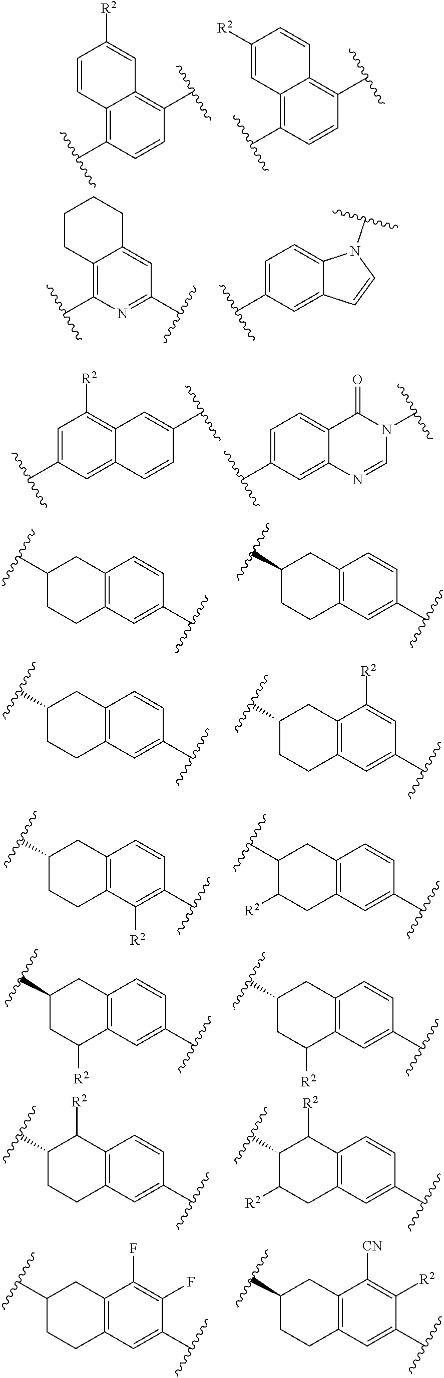

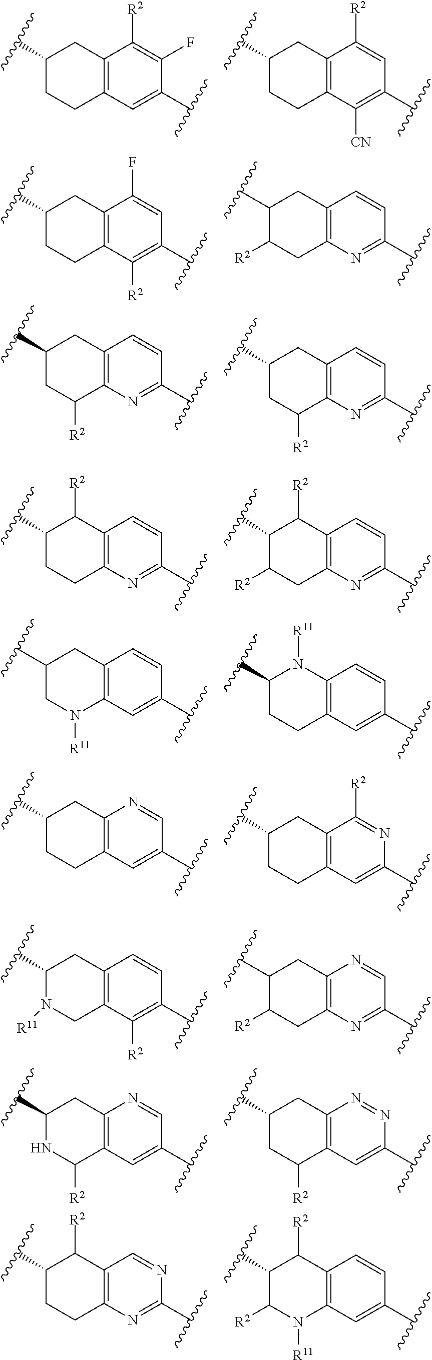

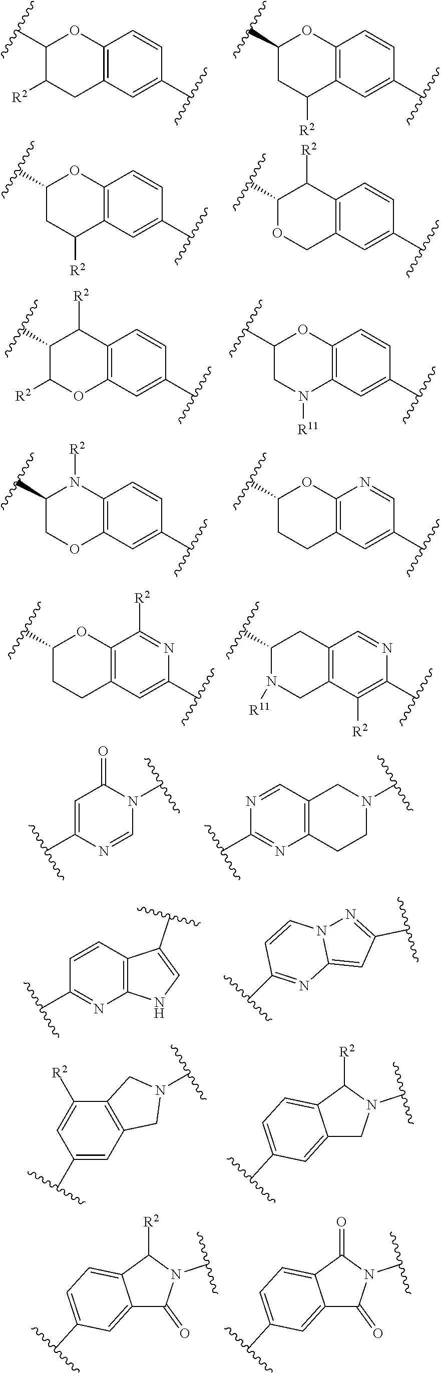

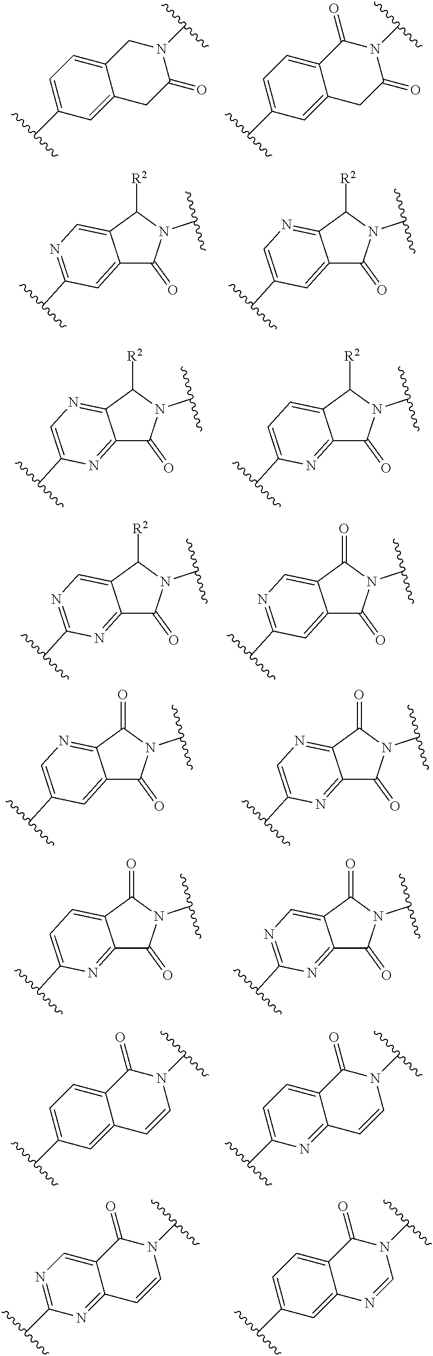

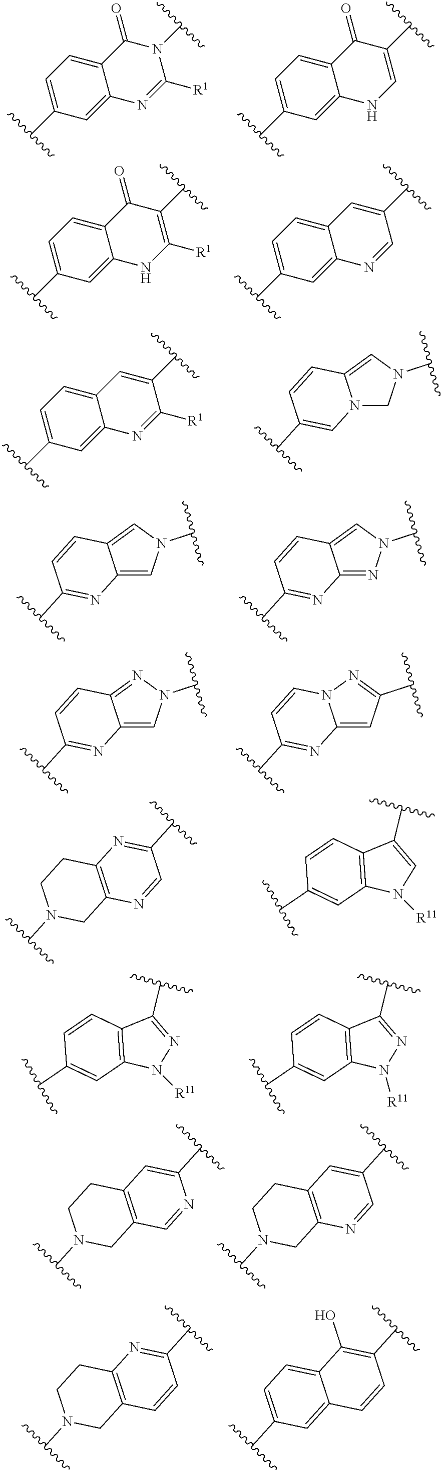

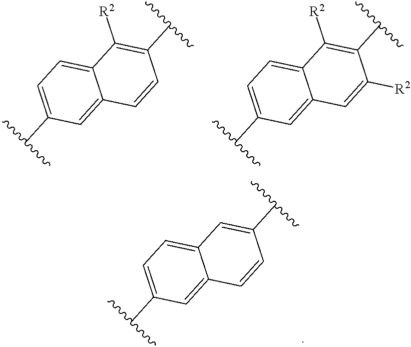

### Embodiments of \textcircled{C}

In certain embodiments

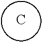

is an aryl group.

In certain embodiments

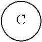

is a phenyl group.

In certain embodiments

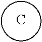

is a heteroaryl group.

In certain embodiments

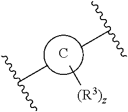

is selected from

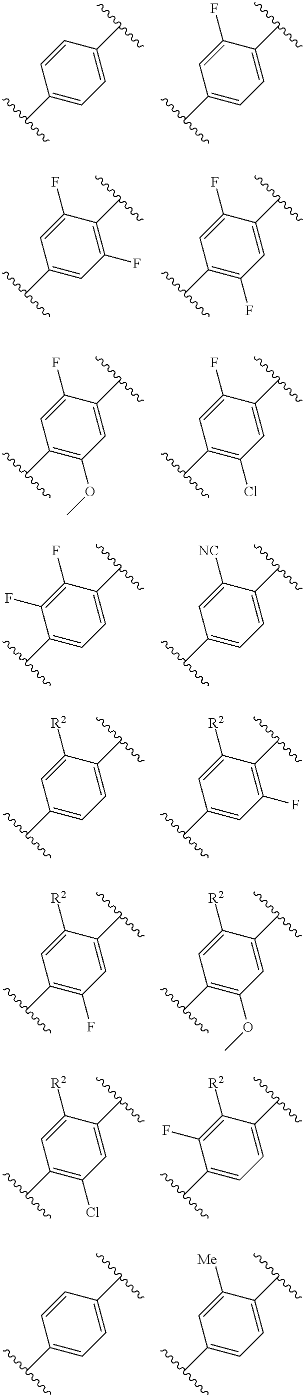

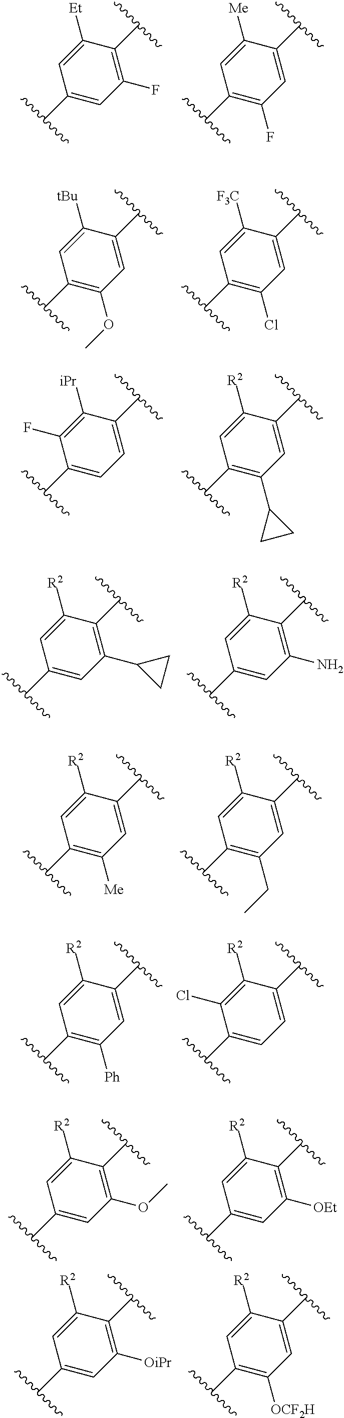

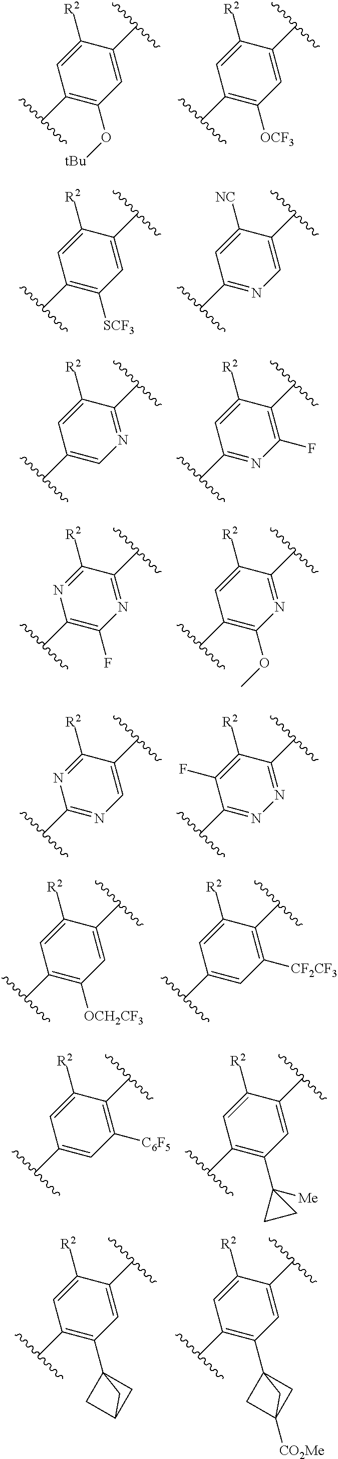

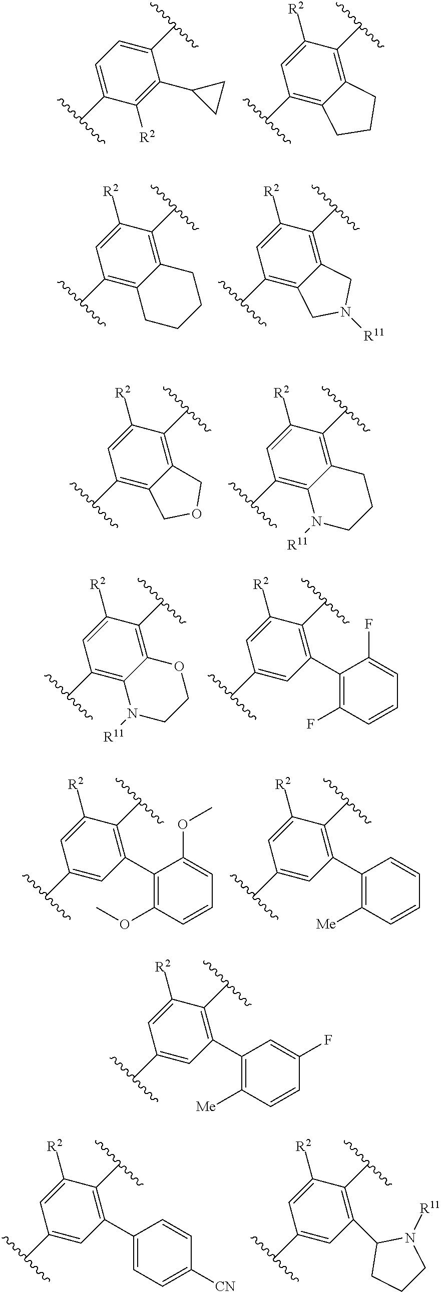

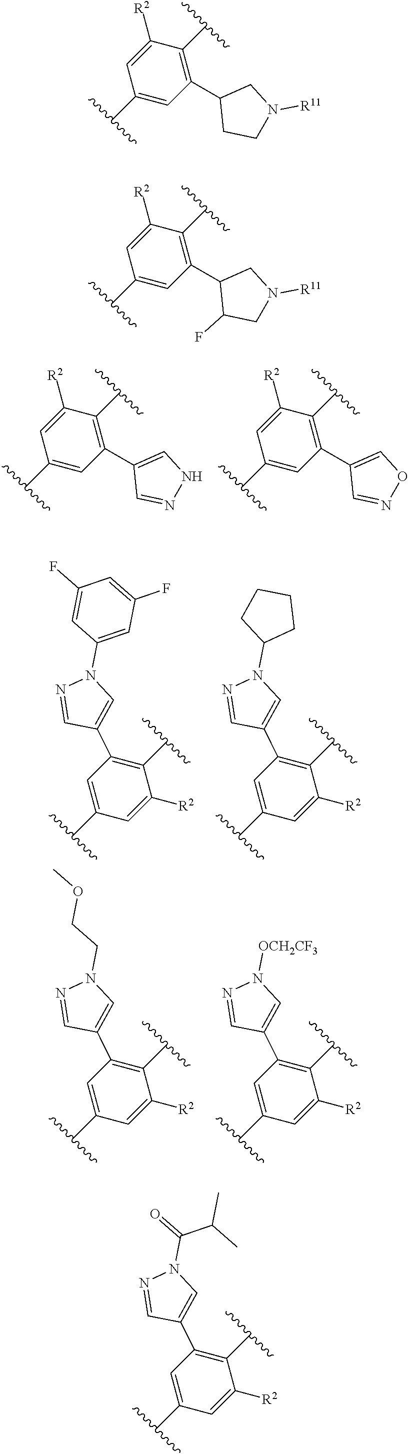

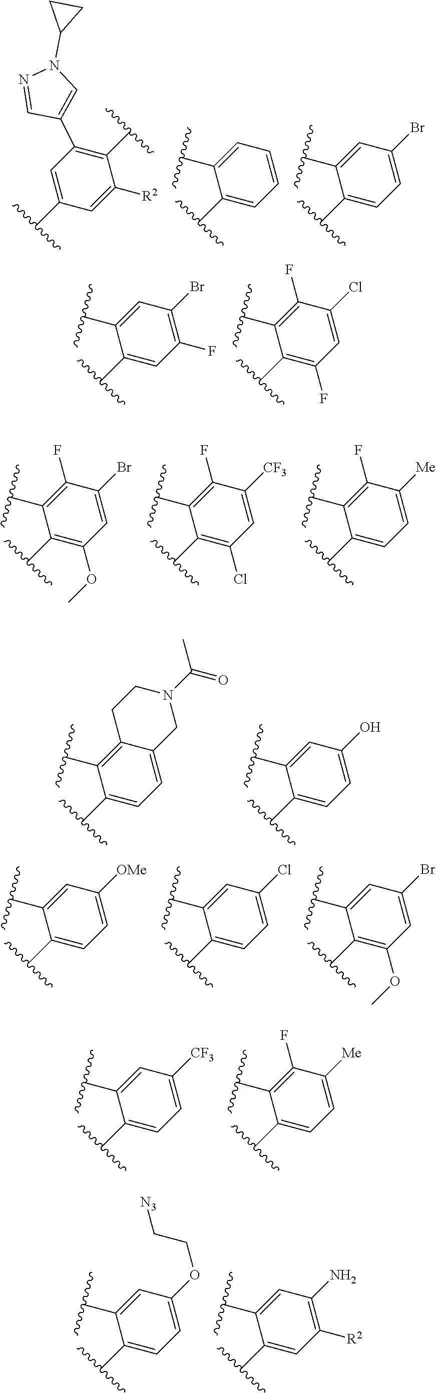

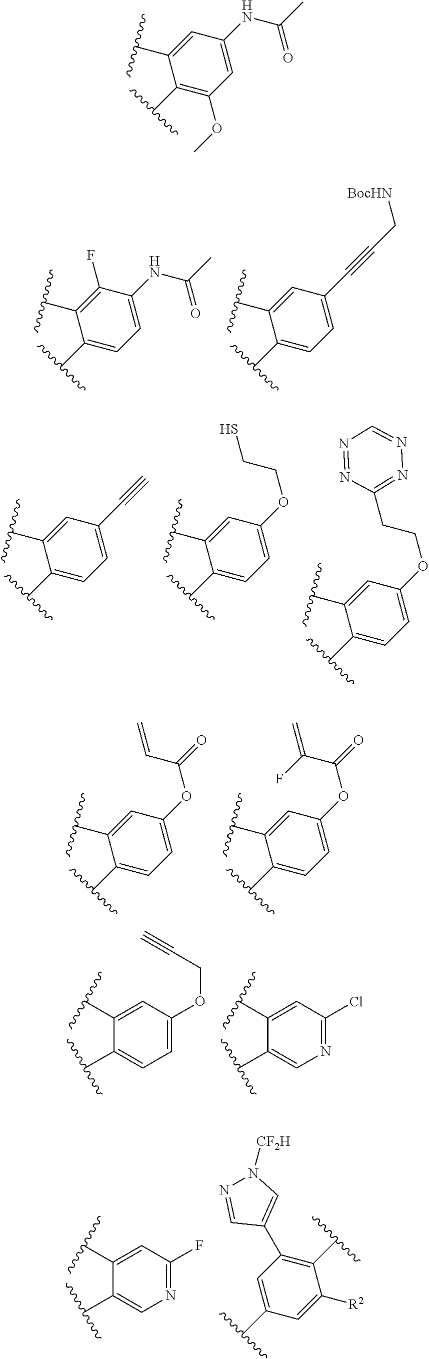

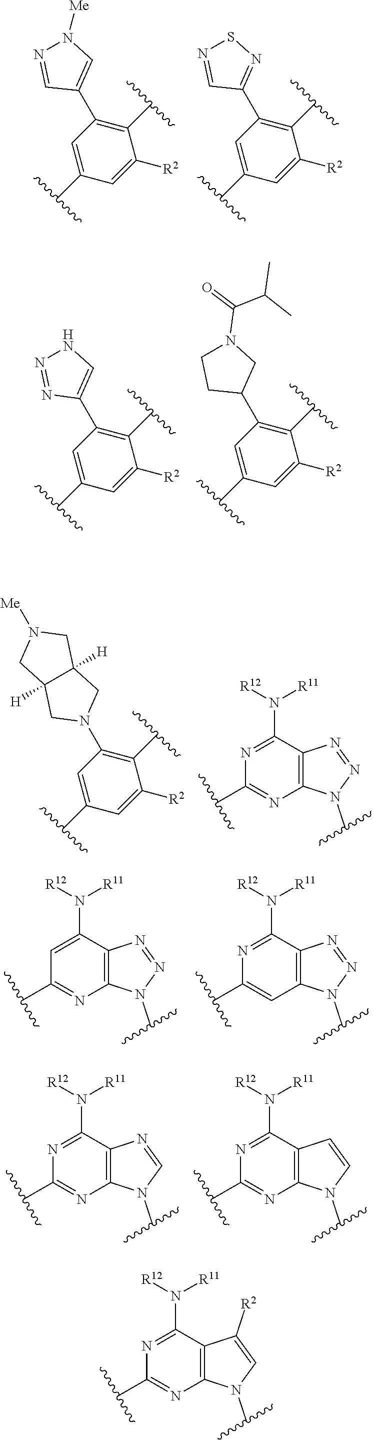

### Embodiments of

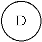

In certain embodiments

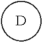

is a heterocycle group.

In certain embodiments

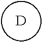

is a substituted piperazine.

In certain embodiments

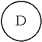

is a substituted bicyclic piperazine group.

In certain embodiments

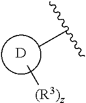

is selected from

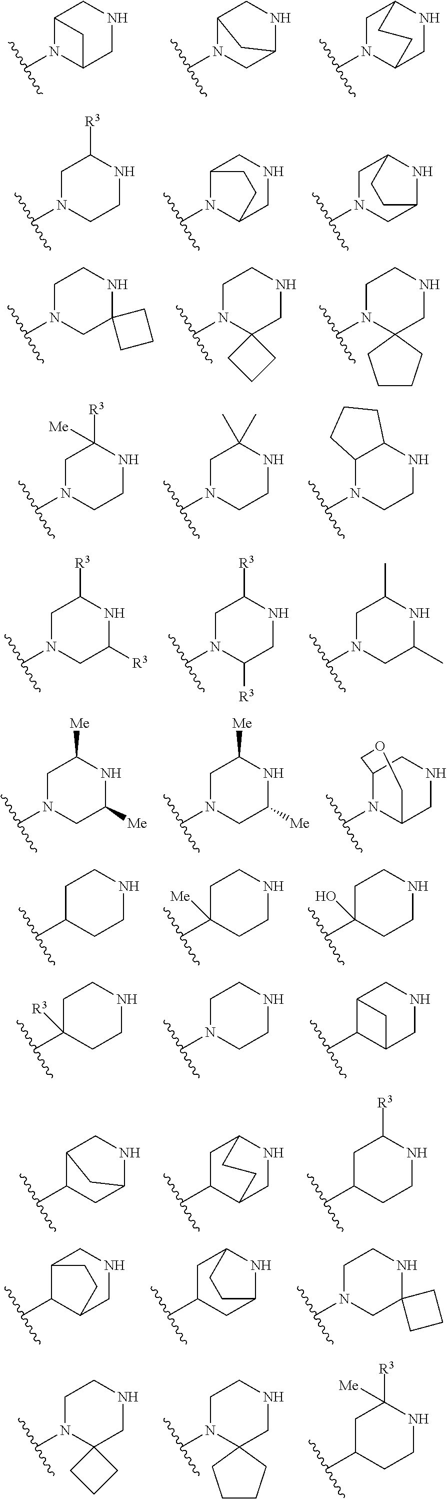

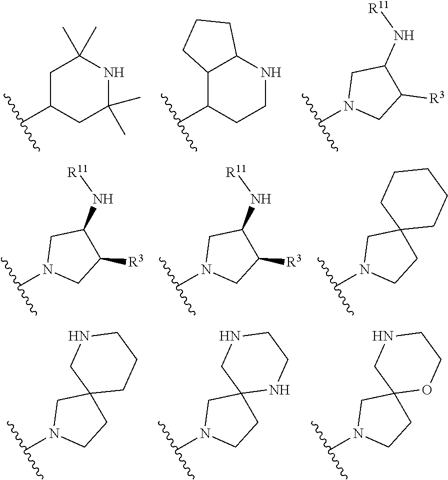

In certain embodiments

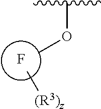

is selected from

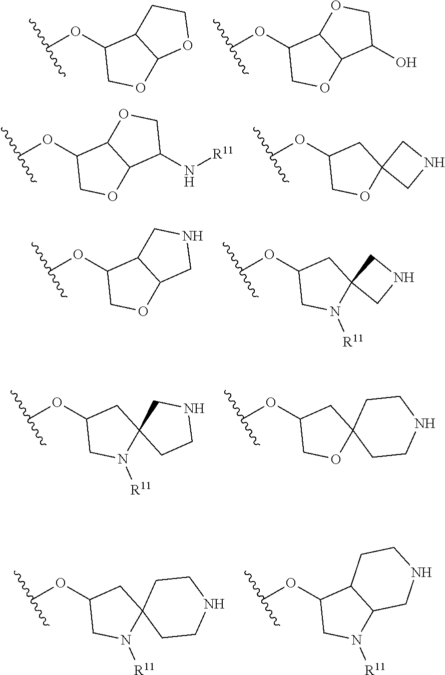

In certain embodiments

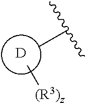

is selected from

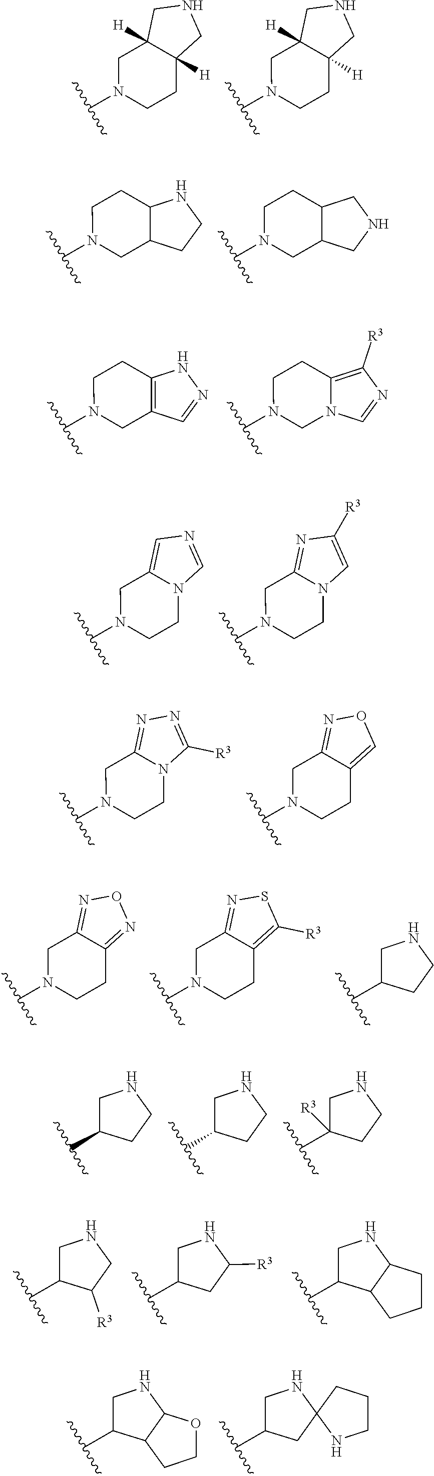

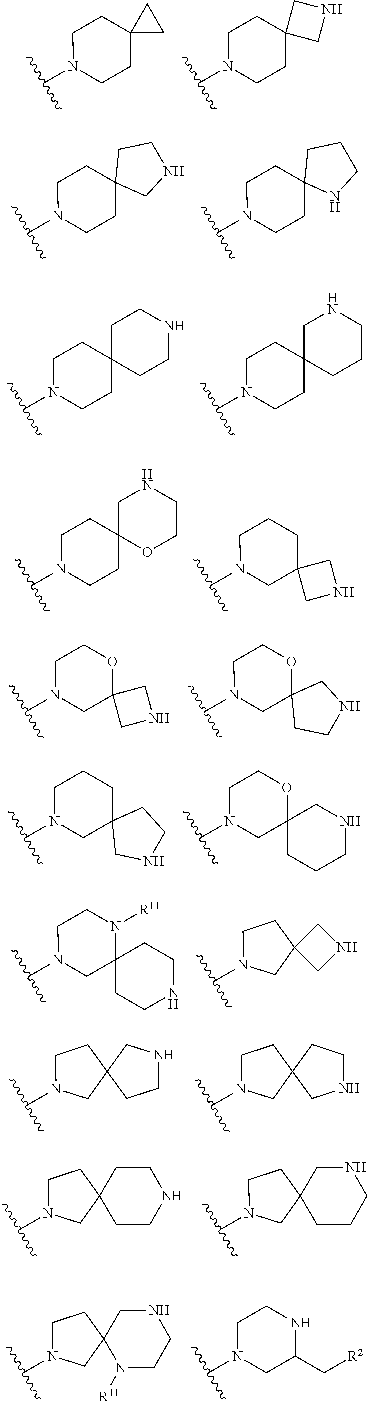

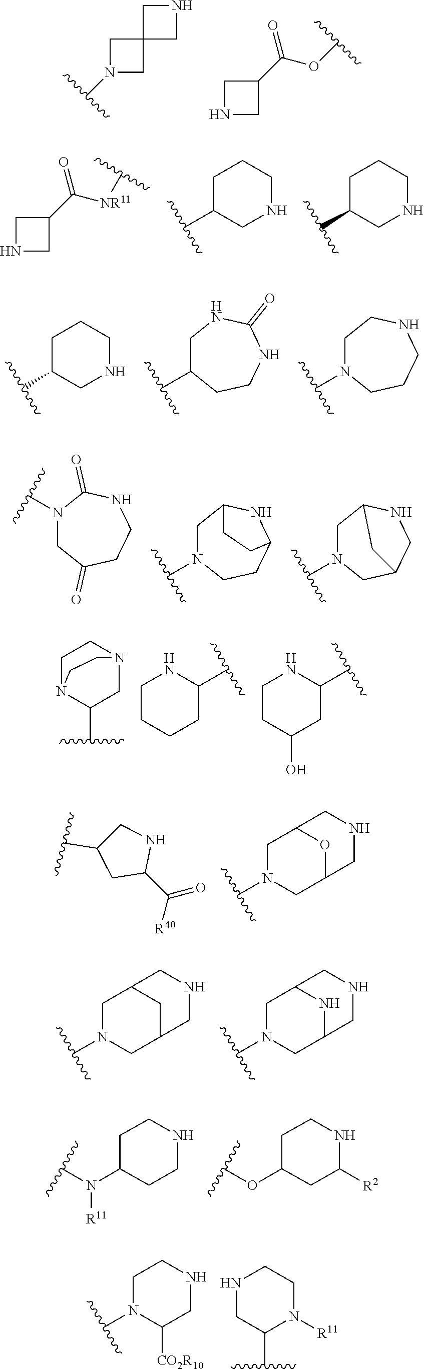

### Embodiments of

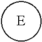

In certain embodiments

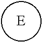

is an aryl group.

In certain embodiments

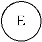

is a phenyl group.

In certain embodiments

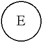

is a heteroaryl group.

In certain embodiments

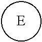

is a heterocycle group.

In certain embodiments

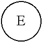

is a cycloalkyl group.

In certain embodiments

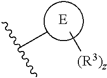

is selected from

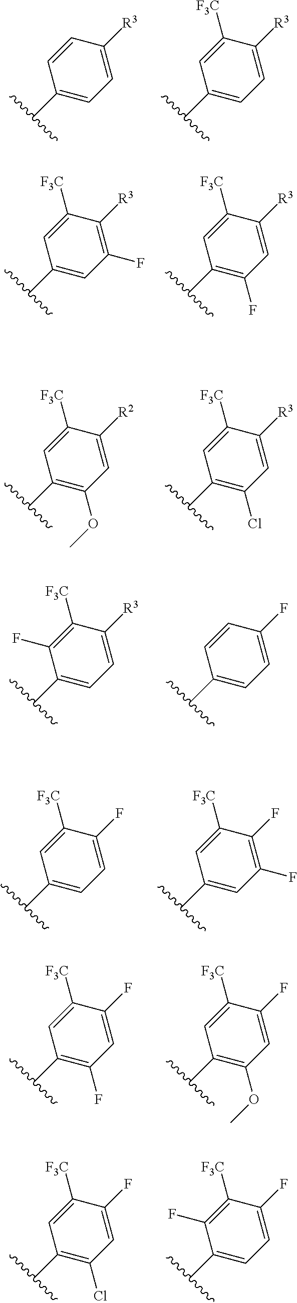

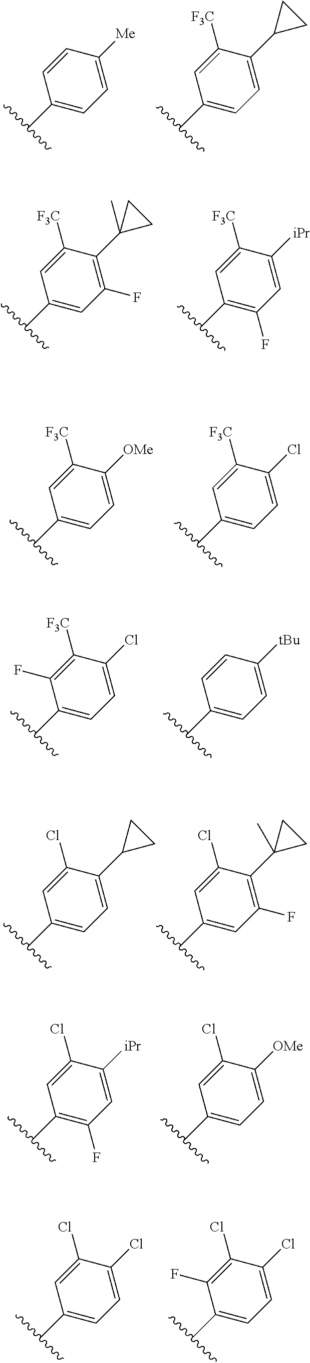

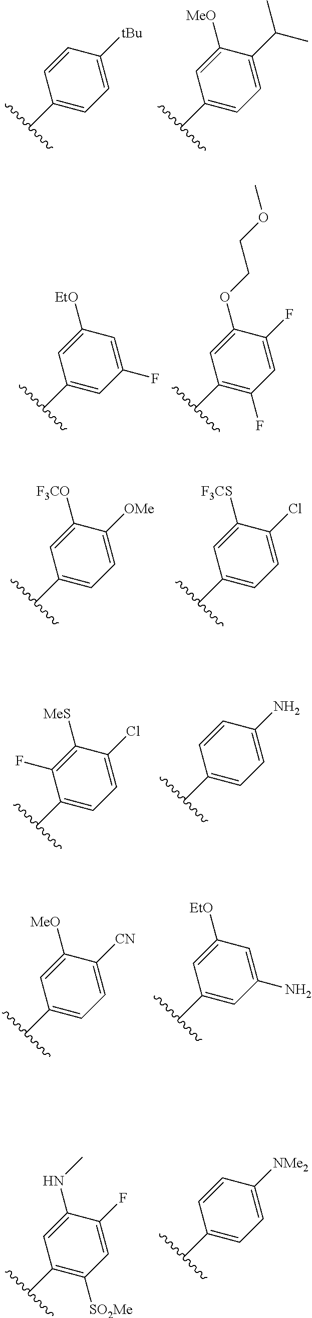

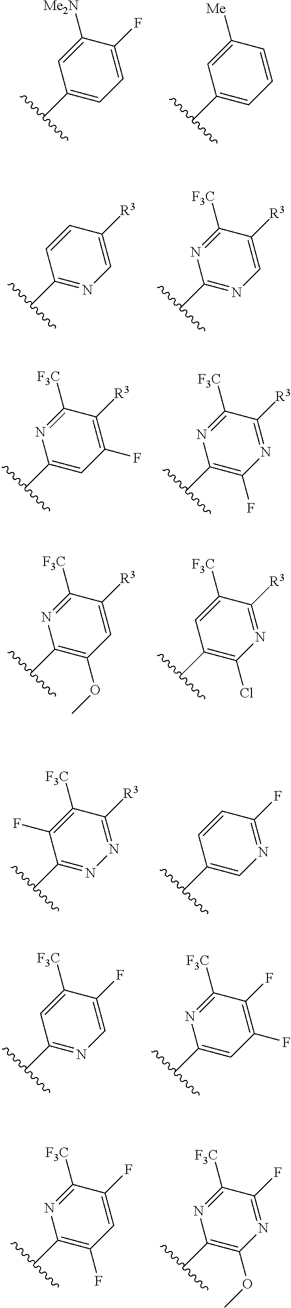

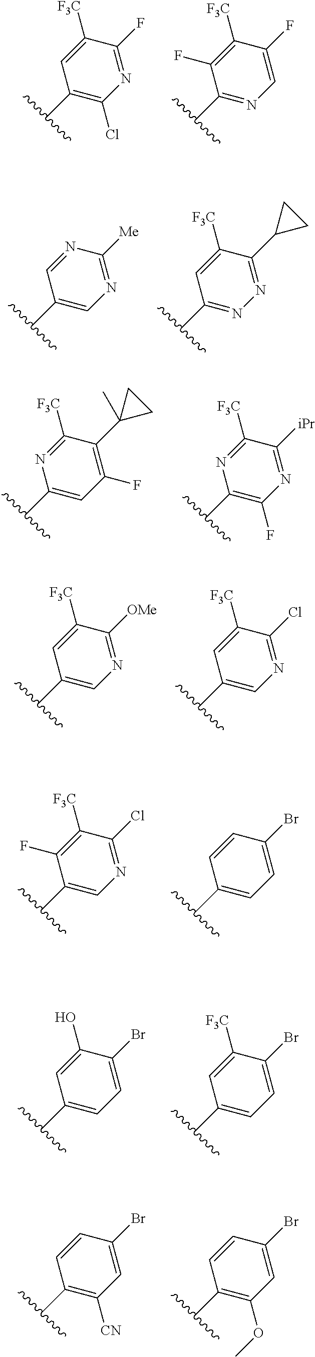

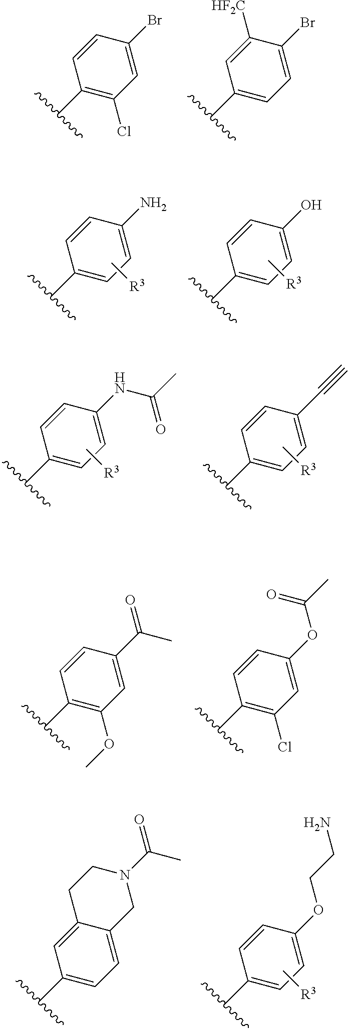

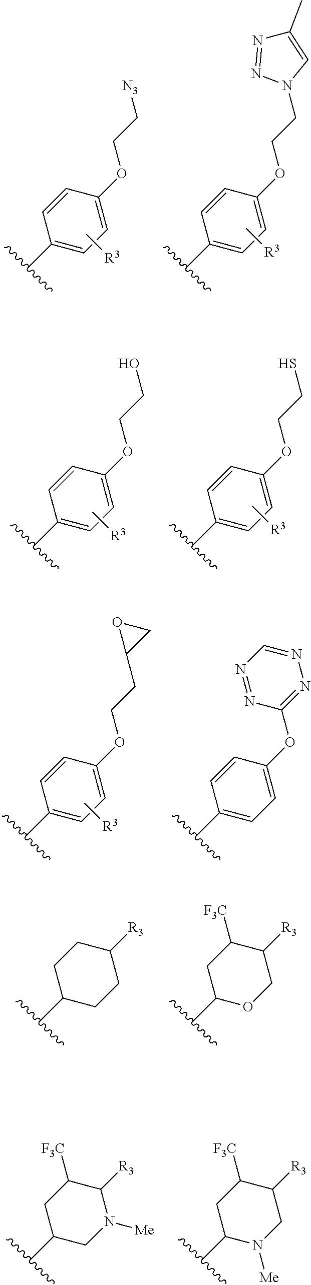

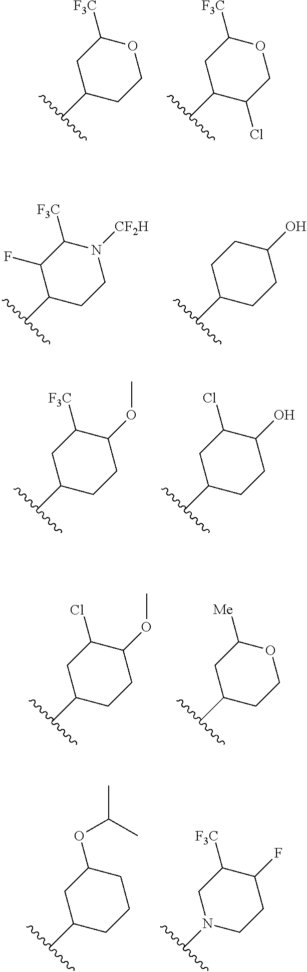

### Embodiments of x, y, and z

In certain embodiments x is 0.

In certain embodiments x is 1.

In certain embodiments x is 2.

In certain embodiments x is 3.

In certain embodiments x is 4.

In certain embodiments y is 0.

In certain embodiments y is 1.

In certain embodiments y is 2.

In certain embodiments y is 3.

In certain embodiments yy is 0.

In certain embodiments yy is 1.

In certain embodiments yy is 2.

In certain embodiments yy is 3.

In certain embodiments z is 0.

In certain embodiments z is 1.

In certain embodiments z is 2.

In certain embodiments z is 3.

In certain embodiments z is 4.

### Embodiments of R¹

In certain embodiments a R¹ is hydrogen.

In certain embodiments one R¹ is hydrogen.

In certain embodiments all R¹ groups are hydrogen.

In certain embodiments a R¹ is halogen.

In certain embodiments one R¹ is halogen.

In certain embodiments a R¹ is alkyl optionally substituted with 1, 2, 3, or 4 substituents selected from R²¹.

In certain embodiments one R¹ is alkyl optionally substituted with 1, 2, 3, or 4 substituents selected from R²¹.

In certain embodiments a R¹ is haloalkyl optionally substituted with 1, 2, 3, or 4 substituents selected from R²¹.

In certain embodiments one R¹ is haloalkyl optionally substituted with 1, 2, 3, or 4 substituents selected from R²¹.

In certain embodiments a R¹ is alkenyl optionally substituted with 1, 2, 3, or 4 substituents selected from R²¹.

In certain embodiments one R¹ is alkenyl optionally substituted with 1, 2, 3, or 4 substituents selected from R²¹.

In certain embodiments a R¹ is alkynyl optionally substituted with 1, 2, 3, or 4 substituents selected from R²¹.

In certain embodiments one R¹ is alkynyl optionally substituted with 1, 2, 3, or 4 substituents selected from R²¹.

In certain embodiments a R¹ is heterocycle optionally substituted with 1, 2, 3, or 4 substituents selected from R²¹.

In certain embodiments one R¹ is heterocycle optionally substituted with 1, 2, 3, or 4 substituents selected from R²¹.

In certain embodiments a R¹ is aryl optionally substituted with 1, 2, 3, or 4 substituents selected from R²¹.

In certain embodiments one R¹ is aryl optionally substituted with 1, 2, 3, or 4 substituents selected from R²¹

In certain embodiments a R¹ is heteroaryl optionally substituted with 1, 2, 3, or 4 substituents selected from R²¹.

In certain embodiments one R¹ is heteroaryl optionally substituted with 1, 2, 3, or 4 substituents selected from R²¹.

In certain embodiments a R¹ is cyano.

In certain embodiments one R¹ is cyano.

In certain embodiments a R¹ is nitro.

In certain embodiments one R¹ is nitro.

In certain embodiments a R¹ is —C(O)R¹⁰.

In certain embodiments one R¹ is —C(O)R¹⁰.

In certain embodiments a R¹ is —OC(O)R¹⁰.

In certain embodiments one R¹ is —OC(O)R¹⁰.

In certain embodiments a R¹ is —NR¹¹C(O)R¹⁰.

In certain embodiments one R¹ is —NR¹¹C(O)R¹⁰.

In certain embodiments a R¹ is —OR¹¹.

In certain embodiments one R¹ is —OR¹¹.

In certain embodiments a R¹ is —NR¹¹R¹².

In certain embodiments one R¹ is —NR¹¹R¹².

In certain embodiments a R¹ is —S(O)R¹⁰.

In certain embodiments one R¹ is —S(O)R¹⁰.

In certain embodiments a R¹ is —S(O)₂R¹⁰.

In certain embodiments one R¹ is —S(O)₂R¹⁰.

In certain embodiments a R¹ is —OS(O)R¹⁰.

In certain embodiments one R¹ is —OS(O)R¹⁰.

In certain embodiments a R¹ is —OS(O)₂R¹⁰.

In certain embodiments one R¹ is —OS(O)₂R¹⁰.

In certain embodiments a R¹ is —NR¹¹S(O)R¹⁰.

In certain embodiments one R¹ is —NR¹¹S(O)R¹⁰.

In certain embodiments a R¹ is —NR¹¹S(O)₂R¹⁰.

In certain embodiments one R¹ is —NR¹¹S(O)₂R¹⁰.

In certain embodiments a R¹ is —SR¹¹.

In certain embodiments one R¹ is —SR¹¹.

### Embodiments of R²

In certain embodiments a R² is hydrogen.

In certain embodiments one R² is hydrogen.

In certain embodiments all R² groups are hydrogen.

In certain embodiments a R² is halogen.

In certain embodiments one R² is halogen.

In certain embodiments a R² is alkyl optionally substituted with 1, 2, 3, or 4 substituents selected from R²².

In certain embodiments one R² is alkyl optionally substituted with 1, 2, 3, or 4 substituents selected from R²².

In certain embodiments a R² is haloalkyl optionally substituted with 1, 2, 3, or 4 substituents selected from R²².

In certain embodiments one R² is haloalkyl optionally substituted with 1, 2, 3, or 4 substituents selected from R²².

In certain embodiments a R² is alkenyl optionally substituted with 1, 2, 3, or 4 substituents selected from R²².

In certain embodiments one R² is alkenyl optionally substituted with 1, 2, 3, or 4 substituents selected from R²².

In certain embodiments a R² is alkynyl optionally substituted with 1, 2, 3, or 4 substituents selected from R²².

In certain embodiments one R² is alkynyl optionally substituted with 1, 2, 3, or 4 substituents selected from R²².

In certain embodiments a R² is heterocycle optionally substituted with 1, 2, 3, or 4 substituents selected from R²².

In certain embodiments one R² is heterocycle optionally substituted with 1, 2, 3, or 4 substituents selected from R²².

In certain embodiments a R² is aryl optionally substituted with 1, 2, 3, or 4 substituents selected from R²².

In certain embodiments one R² is aryl optionally substituted with 1, 2, 3, or 4 substituents selected from R²².

In certain embodiments a R² is heteroaryl optionally substituted with 1, 2, 3, or 4 substituents selected from R²².

In certain embodiments one R² is heteroaryl optionally substituted with 1, 2, 3, or 4 substituents selected from R²².

In certain embodiments a R² is cyano.

In certain embodiments one R² is cyano.

In certain embodiments a R² is nitro.

In certain embodiments one R² is nitro.

In certain embodiments a R² is —C(O)R¹⁰.

In certain embodiments one R² is —C(O)R¹⁰.

In certain embodiments a R² is —OC(O)R¹⁰.

In certain embodiments one R² is —OC(O)R¹⁰.

In certain embodiments a R² is —NR¹¹C(O)R¹⁰.

In certain embodiments one R² is —NR¹¹C(O)R¹⁰.

In certain embodiments a R² is —OR¹¹.

In certain embodiments one R² is —OR¹¹.

In certain embodiments a R² is —NR¹¹R¹².

In certain embodiments one R² is —NR¹¹R¹².

In certain embodiments a R² is —S(O)R¹⁰.

In certain embodiments one R² is —S(O)R¹⁰.

In certain embodiments a R² is —S(O)₂R¹⁰.

In certain embodiments one R² is —S(O)₂R¹⁰.

In certain embodiments a R² is —OS(O)R¹⁰.

In certain embodiments one R² is —OS(O)R¹⁰.

In certain embodiments a R² is —OS(O)₂R¹⁰.

In certain embodiments one R² is —OS(O)₂R¹⁰.

In certain embodiments a R² is —NR¹¹S(O)R¹⁰.

In certain embodiments one R² is —NR¹¹S(O)R¹⁰.

In certain embodiments a R² is —NR¹¹S(O)₂R¹⁰.

In certain embodiments one R² is —NR¹¹S(O)₂R¹⁰.

In certain embodiments a R² is —SR¹¹.

In certain embodiments one R² is —SR¹¹.

### Embodiments of R³

In certain embodiments a R³ is hydrogen.

In certain embodiments one R³ is hydrogen.

In certain embodiments all R³ groups are hydrogen.

In certain embodiments a R³ is halogen.

In certain embodiments one R³ is halogen.

In certain embodiments a R³ is alkyl optionally substituted with 1, 2, 3, or 4 substituents selected from R²³.

In certain embodiments one R³ is alkyl optionally substituted with 1, 2, 3, or 4 substituents selected from R²³.

In certain embodiments a R³ is haloalkyl optionally substituted with 1, 2, 3, or 4 substituents selected from R²³.

In certain embodiments one R³ is haloalkyl optionally substituted with 1, 2, 3, or 4 substituents selected from R²³.

In certain embodiments a R³ is alkenyl optionally substituted with 1, 2, 3, or 4 substituents selected from R²³.

In certain embodiments one R³ is alkenyl optionally substituted with 1, 2, 3, or 4 substituents selected from R²³.

In certain embodiments a R³ is alkynyl optionally substituted with 1, 2, 3, or 4 substituents selected from R²³.

In certain embodiments one R³ is alkynyl optionally substituted with 1, 2, 3, or 4 substituents selected from R²³.

In certain embodiments a R³ is heterocycle optionally substituted with 1, 2, 3, or 4 substituents selected from R²³.

In certain embodiments one R³ is heterocycle optionally substituted with 1, 2, 3, or 4 substituents selected from R²³.

In certain embodiments a R³ is aryl optionally substituted with 1, 2, 3, or 4 substituents selected from R²³.

In certain embodiments one R³ is aryl optionally substituted with 1, 2, 3, or 4 substituents selected from R²³.

In certain embodiments a R³ is heteroaryl optionally substituted with 1, 2, 3, or 4 substituents selected from R²³.

In certain embodiments one R³ is heteroaryl optionally substituted with 1, 2, 3, or 4 substituents selected from R²³.

In certain embodiments a R³ is cyano.

In certain embodiments one R³ is cyano.

In certain embodiments a R³ is nitro.

In certain embodiments one R³ is nitro.

In certain embodiments a R³ is —C(O)R¹⁰.

In certain embodiments one R³ is —C(O)R¹⁰.

In certain embodiments a R³ is —OC(O)R¹⁰.

In certain embodiments one R³ is —OC(O)R¹⁰.

In certain embodiments a R³ is —NR¹¹C(O)R¹⁰.

In certain embodiments one R³ is —NR¹¹C(O)R¹⁰.

In certain embodiments a R³ is —OR¹¹.

In certain embodiments one R³ is —OR¹¹.

In certain embodiments a R³ is —NR¹¹R¹².

In certain embodiments one R³ is —NR¹¹R¹².

In certain embodiments a R³ is —S(O)R¹⁰.

In certain embodiments one R³ is —S(O)R¹⁰.

In certain embodiments a R³ is —S(O)₂R¹⁰.

In certain embodiments one R³ is —S(O)₂R¹⁰.

In certain embodiments a R³ is —OS(O)R¹⁰.

In certain embodiments one R³ is —OS(O)R¹⁰.

In certain embodiments a R³ is —OS(O)₂R¹⁰.

In certain embodiments one R³ is —OS(O)₂R¹⁰.

In certain embodiments a R³ is —NR¹¹S(O)R¹⁰.

In certain embodiments one R³ is —NR¹¹S(O)R¹⁰.

In certain embodiments a R³ is —NR¹¹S(O)₂R¹⁰.

In certain embodiments one R³ is —NR¹¹S(O)₂R¹⁰.

In certain embodiments a R³ is —SR¹¹.

In certain embodiments one R³ is —SR¹¹.

### Embodiments of R⁴

In certain embodiments a R⁴ is hydrogen.

In certain embodiments one R⁴ is hydrogen.

In certain embodiments all R⁴ groups are hydrogen.

In certain embodiments a R⁴ is halogen.

In certain embodiments one R⁴ is halogen.

In certain embodiments a R⁴ is alkyl optionally substituted with 1, 2, 3, or 4 substituents selected from R²⁴.

In certain embodiments one R⁴ is alkyl optionally substituted with 1, 2, 3, or 4 substituents selected from R²⁴.

In certain embodiments a R⁴ is haloalkyl optionally substituted with 1, 2, 3, or 4 substituents selected from R²⁴.

In certain embodiments one R⁴ is haloalkyl optionally substituted with 1, 2, 3, or 4 substituents selected from R²⁴.

In certain embodiments a R⁴ is alkenyl optionally substituted with 1, 2, 3, or 4 substituents selected from R²⁴.

In certain embodiments one R⁴ is alkenyl optionally substituted with 1, 2, 3, or 4 substituents selected from R²⁴.

In certain embodiments a R⁴ is alkynyl optionally substituted with 1, 2, 3, or 4 substituents selected from R²⁴.

In certain embodiments one R⁴ is alkynyl optionally substituted with 1, 2, 3, or 4 substituents selected from R²⁴.

In certain embodiments a R⁴ is heterocycle optionally substituted with 1, 2, 3, or 4 substituents selected from R²⁴.

In certain embodiments one R⁴ is heterocycle optionally substituted with 1, 2, 3, or 4 substituents selected from R²⁴.

In certain embodiments a R⁴ is aryl optionally substituted with 1, 2, 3, or 4 substituents selected from R²⁴.

In certain embodiments one R⁴ is aryl optionally substituted with 1, 2, 3, or 4 substituents selected from R²⁴.

In certain embodiments a R⁴ is heteroaryl optionally substituted with 1, 2, 3, or 4 substituents selected from R²⁴.

In certain embodiments one R⁴ is heteroaryl optionally substituted with 1, 2, 3, or 4 substituents selected from R²⁴.

In certain embodiments a R⁴ is cyano.

In certain embodiments one R⁴ is cyano.

In certain embodiments a R⁴ is nitro.

In certain embodiments one R⁴ is nitro.

In certain embodiments a R⁴ is —C(O)R¹⁰.

In certain embodiments one R⁴ is —C(O)R¹⁰.

In certain embodiments a R⁴ is —OC(O)R¹⁰.

In certain embodiments one R⁴ is —OC(O)R¹⁰.

In certain embodiments a R⁴ is —NR¹¹C(O)R¹⁰.

In certain embodiments one R⁴ is —NR¹¹C(O)R¹⁰.

In certain embodiments a R⁴ is —OR¹¹.

In certain embodiments one R⁴ is —OR¹¹.

In certain embodiments a R⁴ is —NR¹¹R¹².

In certain embodiments one R⁴ is —NR¹¹R¹².

In certain embodiments a R⁴ is —S(O)R¹⁰.

In certain embodiments one R⁴ is —S(O)R¹⁰.

In certain embodiments a R⁴ is —S(O)₂R¹⁰.

In certain embodiments one R⁴ is —S(O)₂R¹⁰.

In certain embodiments a R⁴ is —OS(O)R¹⁰.

In certain embodiments one R⁴ is —OS(O)R¹⁰.

In certain embodiments a R⁴ is —OS(O)₂R¹⁰.

In certain embodiments one R⁴ is —OS(O)₂R¹⁰.

In certain embodiments a R⁴ is —NR¹¹S(O)R¹⁰.

In certain embodiments one R⁴ is —NR¹¹S(O)R¹⁰.

In certain embodiments a R⁴ is —NR¹¹S(O)₂R¹⁰.

In certain embodiments one R⁴ is —NR¹¹S(O)₂R¹⁰.

In certain embodiments a R⁴ is —SR¹¹.

In certain embodiments one R⁴ is —SR¹¹.

### Embodiments of R⁵

In certain embodiments a R⁵ is hydrogen.

In certain embodiments one R⁵ is hydrogen.

In certain embodiments all R⁵ groups are hydrogen.

In certain embodiments a R⁵ is halogen.

In certain embodiments one R⁵ is halogen.

In certain embodiments a R⁵ is alkyl optionally substituted with 1, 2, 3, or 4 substituents selected from R²⁵.

In certain embodiments one R⁵ is alkyl optionally substituted with 1, 2, 3, or 4 substituents selected from R²⁵.

In certain embodiments a R⁵ is haloalkyl optionally substituted with 1, 2, 3, or 4 substituents selected from R²⁵.

In certain embodiments one R⁵ is haloalkyl optionally substituted with 1, 2, 3, or 4 substituents selected from R²⁵.

In certain embodiments a R⁵ is alkenyl optionally substituted with 1, 2, 3, or 4 substituents selected from R²⁵.

In certain embodiments one R⁵ is alkenyl optionally substituted with 1, 2, 3, or 4 substituents selected from R²⁵.

In certain embodiments a R⁵ is alkynyl optionally substituted with 1, 2, 3, or 4 substituents selected from R²⁵.

In certain embodiments one R⁵ is alkynyl optionally substituted with 1, 2, 3, or 4 substituents selected from R²⁵.

In certain embodiments a R⁵ is heterocycle optionally substituted with 1, 2, 3, or 4 substituents selected from R²⁵.

In certain embodiments one R⁵ is heterocycle optionally substituted with 1, 2, 3, or 4 substituents selected from R²⁵.

In certain embodiments a R⁵ is aryl optionally substituted with 1, 2, 3, or 4 substituents selected from R²⁵.

In certain embodiments one R⁵ is aryl optionally substituted with 1, 2, 3, or 4 substituents selected from R²⁵.

In certain embodiments a R⁵ is heteroaryl optionally substituted with 1, 2, 3, or 4 substituents selected from R²⁵.

In certain embodiments one R⁵ is heteroaryl optionally substituted with 1, 2, 3, or 4 substituents selected from R²⁵.

In certain embodiments a R⁵ is cyano.

In certain embodiments one R⁵ is cyano.

In certain embodiments a R⁵ is nitro.

In certain embodiments one R⁵ is nitro.

In certain embodiments a R⁵ is —C(O)R¹⁰.

In certain embodiments one R⁵ is —C(O)R¹⁰.

In certain embodiments a R⁵ is —OC(O)R¹⁰.

In certain embodiments one R⁵ is —OC(O)R¹⁰.

In certain embodiments a R⁵ is —NR¹¹C(O)R¹⁰.

In certain embodiments one R⁵ is —NR¹¹C(O)R¹⁰.

In certain embodiments a R⁵ is —OR¹¹.

In certain embodiments one R⁵ is —OR¹¹.

In certain embodiments a R^{S} is —NR¹¹R¹².

In certain embodiments one R^{S} is —NR¹¹R¹².

In certain embodiments a R⁵ is —S(O)R¹⁰.

In certain embodiments one R⁵ is —S(O)R¹⁰.

In certain embodiments a R⁵ is —S(O)₂R¹⁰.

In certain embodiments one R⁵ is —S(O)₂R¹⁰.

In certain embodiments a R⁵ is —OS(O)R¹⁰.

In certain embodiments one R⁵ is —OS(O)R¹⁰.

In certain embodiments a R⁵ is —OS(O)₂R¹⁰.

In certain embodiments one R⁵ is —OS(O)₂R¹⁰.

In certain embodiments a R⁵ is —NR¹¹S(O)R¹⁰.

In certain embodiments one R⁵ is —NR¹¹S(O)R¹⁰.

In certain embodiments a R⁵ is —NR¹¹S(O)₂R¹⁰.

In certain embodiments one R⁵ is —NR¹¹S(O)₂R¹⁰.

In certain embodiments a R⁵ is —SR¹¹.

In certain embodiments one R⁵ is —SR¹¹.

### Embodiments of R⁶

In certain embodiments a R⁶ is hydrogen.

In certain embodiments one R⁶ is hydrogen.

In certain embodiments all R⁶ groups are hydrogen.

In certain embodiments a R⁶ is halogen.

In certain embodiments one R⁶ is halogen.

In certain embodiments a R⁶ is alkyl optionally substituted with 1, 2, 3, or 4 substituents selected from R²⁶.

In certain embodiments one R⁶ is alkyl optionally substituted with 1, 2, 3, or 4 substituents selected from R²⁶.

In certain embodiments a R⁶ is haloalkyl optionally substituted with 1, 2, 3, or 4 substituents selected from R²⁶.

In certain embodiments one R⁶ is haloalkyl optionally substituted with 1, 2, 3, or 4 substituents selected from R²⁶.

In certain embodiments a R⁶ is alkenyl optionally substituted with 1, 2, 3, or 4 substituents selected from R²⁶.

In certain embodiments one R⁶ is alkenyl optionally substituted with 1, 2, 3, or 4 substituents selected from R²⁶.

In certain embodiments a R⁶ is alkynyl optionally substituted with 1, 2, 3, or 4 substituents selected from R²⁶.

In certain embodiments one R⁶ is alkynyl optionally substituted with 1, 2, 3, or 4 substituents selected from R²⁶.

In certain embodiments a R⁶ is heterocycle optionally substituted with 1, 2, 3, or 4 substituents selected from R²⁶.

In certain embodiments one R⁶ is heterocycle optionally substituted with 1, 2, 3, or 4 substituents selected from R²⁶.

In certain embodiments a R⁶ is aryl optionally substituted with 1, 2, 3, or 4 substituents selected from R²⁶.

In certain embodiments one R⁶ is aryl optionally substituted with 1, 2, 3, or 4 substituents selected from R²⁶.

In certain embodiments a R⁶ is heteroaryl optionally substituted with 1, 2, 3, or 4 substituents selected from R²⁶.

In certain embodiments one R⁶ is heteroaryl optionally substituted with 1, 2, 3, or 4 substituents selected from R²⁶.

In certain embodiments a R⁶ is cyano.

In certain embodiments one R⁶ is cyano.

In certain embodiments a R⁶ is nitro.

In certain embodiments one R⁶ is nitro.

In certain embodiments a R⁶ is —C(O)R¹⁰.

In certain embodiments one R⁶ is —C(O)R¹⁰.

In certain embodiments a R⁶ is —OC(O)R¹⁰.

In certain embodiments one R⁶ is —OC(O)R¹⁰.

In certain embodiments a R⁶ is —NR¹¹C(O)R¹⁰.

In certain embodiments one R⁶ is —NR¹¹C(O)R¹⁰.

In certain embodiments a R⁶ is —OR¹¹.

In certain embodiments one R⁶ is —OR¹¹.

In certain embodiments a R⁶ is —NR¹¹R¹².

In certain embodiments one R⁶ is —NR¹¹R¹².

In certain embodiments a R⁶ is —S(O)R¹⁰.

In certain embodiments one R⁶ is —S(O)R¹⁰.

In certain embodiments a R⁶ is —S(O)₂R¹⁰.

In certain embodiments one R⁶ is —S(O)₂R¹⁰.

In certain embodiments a R⁶ is —OS(O)R¹⁰.

In certain embodiments one R⁶ is —OS(O)R¹⁰.

In certain embodiments a R⁶ is —OS(O)₂R¹⁰.

In certain embodiments one R⁶ is —OS(O)₂R¹⁰.

In certain embodiments a R⁶ is —NR¹¹S(O)R¹⁰.

In certain embodiments one R⁶ is —NR¹¹S(O)R¹⁰.

In certain embodiments a R⁶ is —NR¹¹S(O)₂R¹⁰.

In certain embodiments one R⁶ is —NR¹¹S(O)₂R¹⁰.

In certain embodiments a R⁶ is —SR¹¹.

In certain embodiments one R⁶ is —SR¹¹.

### Embodiments of R¹¹

In certain embodiments R¹⁰ is independently selected at each instance from hydrogen, and alkyl.

In certain embodiments each R¹⁰ is hydrogen.

In certain embodiments each R¹⁰ is alkyl.

In certain embodiments each R¹⁰ is methyl.

In certain embodiments a R¹⁰ is alkyl optionally substituted as allowed by valence with 1, 2, 3, or 4 substituents selected from R³⁰.

In certain embodiments a R¹⁰ is haloalkyl optionally substituted as allowed by valence with 1, 2, 3, or 4 substituents selected from R³⁰.

In certain embodiments a R¹⁰ is alkenyl or alkynyl optionally substituted as allowed by valence with 1, 2, 3, or 4 substituents selected from R³⁰.

In certain embodiments a R¹⁰ is —OR¹¹.

In certain embodiments a R¹⁰ is —NR¹¹R¹².

In certain embodiments a R¹⁰ is —SR¹¹.

In certain embodiments a R¹⁰ is aryl optionally substituted as allowed by valence with 1, 2, 3, or 4 substituents selected from R³⁰.

In certain embodiments a R¹⁰ is phenyl optionally substituted as allowed by valence with 1, 2, 3, or 4 substituents selected from R³⁰.

In certain embodiments a R¹⁰ is heterocycle optionally substituted as allowed by valence with 1, 2, 3, or 4 substituents selected from R³¹.

In certain embodiments a R¹⁰ is heteroaryl optionally substituted as allowed by valence with 1, 2, 3, or 4 substituents selected from R³¹.

### Embodiments of R¹¹ and R¹²

In certain embodiments R¹¹ and R¹² are hydrogen.

In certain embodiments a R¹¹ is hydrogen.

In certain embodiments a R¹² is hydrogen.

In certain embodiments R¹¹ and R¹² are alkyl.

In certain embodiments a R¹¹ is alkyl.

In certain embodiments a R¹² is alkyl.

In certain embodiments R¹¹ and R¹² are methyl.

In certain embodiments a R¹¹ is methyl.

In certain embodiments a R¹² is methyl.

In certain embodiments R¹¹ or R¹² is haloalkyl optionally substituted as allowed by valence with 1, 2, 3, or 4 substituents selected from R³¹.

In certain embodiments R¹¹ or R¹² is alkenyl or alkynyl optionally substituted as allowed by valence with 1, 2, 3, or 4 substituents selected from R³¹.

In certain embodiments R¹¹ or R¹² is aryl optionally substituted as allowed by valence with 1, 2, 3, or 4 substituents selected from R³¹.

In certain embodiments R¹¹ or R¹² is phenyl optionally substituted as allowed by valence with 1, 2, 3, or 4 substituents selected from R³¹.

In certain embodiments R¹¹ or R¹² is heterocycle optionally substituted as allowed by valence with 1, 2, 3, or 4 substituents selected from R³¹.

In certain embodiments R¹¹ or R¹² is heteroaryl optionally substituted as allowed by valence with 1, 2, 3, or 4 substituents selected from R³¹.

In certain embodiments R¹¹ or R¹² is —C(O)R⁴⁰ optionally substituted as allowed by valence with 1, 2, 3, or 4 substituents selected from R³¹.

In certain embodiments R¹¹ or R¹² is —S(O)R⁴⁰ optionally substituted as allowed by valence with 1, 2, 3, or 4 substituents selected from R³¹.

In certain embodiments R¹¹ or R¹² is —S(O)₂R⁴⁰ optionally substituted as allowed by valence with 1, 2, 3, or 4 substituents selected from R³¹.

### Embodiments of R²¹, R²², R²³, R²⁴, R²⁵, and R²⁶

In certain embodiments R²¹, R²², R²³, R²⁴, R²⁵, and R²⁶ are selected at each instance from hydrogen, halogen, alkyl, and haloalkyl.

In certain embodiments at least one of R²¹, R²², R²³, R²⁴, R²⁵, and R²⁶ is halogen.

In certain embodiments at least one of R²¹, R²², R²³, R²⁴, R²⁵, and R²⁶ is alkyl optionally substituted as allowed by valence with 1, 2, 3, or 4 substituents selected from R⁴³.

In certain embodiments at least one of R²¹, R²², R²³, R²⁴, R²⁵, and R²⁶ is haloalkyl optionally substituted as allowed by valence with 1, 2, 3, or 4 substituents selected from R⁴³.

In certain embodiments at least one of R²¹, R²², R²³, R²⁴, R²⁵, and R²⁶ is alkenyl optionally substituted as allowed by valence with 1, 2, 3, or 4 substituents selected from R⁴³.

In certain embodiments at least one of R²¹, R²², R²³, R²⁴, R²⁵, and R²⁶ is alkynyl optionally substituted as allowed by valence with 1, 2, 3, or 4 substituents selected from R⁴³.

In certain embodiments at least one of R²¹, R²², R²³, R²⁴, R²⁵, and R²⁶ is heterocycle optionally substituted as allowed by valence with 1, 2, 3, or 4 substituents selected from R⁴³.

In certain embodiments at least one of R²¹, R²², R²³, R²⁴, R²⁵, and R²⁶ is aryl optionally substituted as allowed by valence with 1, 2, 3, or 4 substituents selected from R⁴³.

In certain embodiments at least one of R²¹, R²², R²³, R²⁴, R²⁵, and R²⁶ is heteroaryl optionally substituted as allowed by valence with 1, 2, 3, or 4 substituents selected from R⁴³.

In certain embodiments at least one of R²¹, R²², R²³, R²⁴, R²⁵, and R²⁶ is cyano.

In certain embodiments at least one of R²¹, R²², R²³, R²⁴, R²⁵, and R²⁶ is nitro.

In certain embodiments at least one of R²¹, R²², R²³, R²⁴, R²⁵, and R²⁶ is —C(O)R⁴⁰.

In certain embodiments at least one of R²¹, R²², R²³, R²⁴, R²⁵, and R²⁶ is —OC(O)R⁴⁰.

In certain embodiments at least one of R²¹, R²², R²³, R²⁴, R²⁵, and R²⁶ is —NR⁴¹C(O)R⁴⁰.

In certain embodiments at least one of R²¹, R²², R²³, R²⁴, R²⁵, and R²⁶ is —OR⁴¹.

In certain embodiments at least one of R²¹, R²², R²³, R²⁴, R²⁵, and R²⁶ is —NR⁴¹R⁴².

In certain embodiments at least one of R²¹, R²², R²³, R²⁴, R²⁵, and R²⁶ is —S(O)R⁴⁰.

In certain embodiments at least one of R²¹, R²², R²³, R²⁴, R²⁵, and R²⁶ is —OS(O)R⁴⁰.

In certain embodiments at least one of R²¹, R²², R²³, R²⁴, R²⁵, and R²⁶ is —OS(O)₂R⁴⁰.

In certain embodiments at least one of R²¹, R²², R²³, R²⁴, R²⁵, and R²⁶ is —NR⁴¹S(O)R⁴⁰.

In certain embodiments at least one of R²¹, R²², R²³, R²⁴, R²⁵, and R²⁶ is —NR⁴¹S(O)₂R⁴⁰.

In certain embodiments at least one of R²¹, R²², R²³, R²⁴, R²⁵, and R²⁶ is —SR⁴¹.

### Embodiments of R³⁰ and R³¹

In certain embodiments R³⁰ or R³¹ is hydrogen.

In certain embodiments R³⁰ or R³¹ is halogen.

In certain embodiments R³⁰ or R³¹ is alkyl optionally substituted as allowed by valence with 1, 2, 3, or 4 substituents selected from R⁴³.

In certain embodiments R³⁰ or R³¹ is haloalkyl optionally substituted as allowed by valence with 1, 2, 3, or 4 substituents selected from R⁴³.

In certain embodiments R³⁰ or R³¹ is alkenyl optionally substituted as allowed by valence with 1, 2, 3, or 4 substituents selected from R⁴³.

In certain embodiments R³⁰ or R³¹ is alkynyl optionally substituted as allowed by valence with 1, 2, 3, or 4 substituents selected from R⁴³.

In certain embodiments R³⁰ or R³¹ is heterocycle optionally substituted as allowed by valence with 1, 2, 3, or 4 substituents selected from R⁴³.

In certain embodiments R³⁰ or R³¹ is aryl optionally substituted as allowed by valence with 1, 2, 3, or 4 substituents selected from R⁴³.

In certain embodiments R³⁰ or R³¹ is heteroaryl optionally substituted as allowed by valence with 1, 2, 3, or 4 substituents selected from R⁴³.

In certain embodiments R³⁰ or R³¹ is cyano.

In certain embodiments R³⁰ or R³¹ is nitro.

In certain embodiments R³⁰ or R³¹ is —C(O)R⁴⁰.

In certain embodiments R³⁰ or R³¹ is —OC(O)R⁴⁰.

In certain embodiments R³⁰ or R³¹ is —NR⁴¹C(O)R⁴⁰.

In certain embodiments R³⁰ or R³¹ is —OR⁴¹.

In certain embodiments R³⁰ or R³¹ is —NR⁴¹R⁴².

In certain embodiments R³⁰ or R³¹ is —S(O)R⁴⁰.

In certain embodiments R³⁰ or R³¹ is —S(O)₂R⁴⁰.

In certain embodiments R³⁰ or R³¹ is —OS(O)R⁴⁰.

In certain embodiments R³⁰ or R³¹ is —OS(O)₂R⁴⁰.

In certain embodiments R³⁰ or R³¹ is —NR⁴¹S(O)R⁴⁰.

In certain embodiments R³⁰ or R³¹ is —NR⁴¹S(O)₂R⁴⁰.

In certain embodiments R³⁰ or R³¹ is —SR⁴¹.

### Embodiments of R⁴⁰

In certain embodiments a R⁴⁰ is hydrogen.

In certain embodiments a R⁴⁰ is alkyl optionally substituted as allowed by valence with 1, 2, 3, or 4 substituents selected from R⁴³.

In certain embodiments a R⁴⁰ is haloalkyl optionally substituted as allowed by valence with 1, 2, 3, or 4 substituents selected from R⁴³.

In certain embodiments a R⁴⁰ is alkenyl optionally substituted as allowed by valence with 1, 2, 3, or 4 substituents selected from R⁴³.

In certain embodiments a R⁴⁰ is alkynyl optionally substituted as allowed by valence with 15 1, 2, 3, or 4 substituents selected from R⁴³.

In certain embodiments a R⁴⁰ is aryl optionally substituted as allowed by valence with 1, 2, 3, or 4 substituents selected from R⁴³.

In certain embodiments a R⁴⁰ is heterocycle optionally substituted as allowed by valence with 1, 2, 3, or 4 substituents selected from R⁴³.

In certain embodiments a R⁴⁰ is heteroaryl optionally substituted as allowed by valence with 1, 2, 3, or 4 substituents selected from R⁴³.

In certain embodiments a R⁴⁰ is amino optionally substituted as allowed by valence with 1, 2, 3, or 4 substituents selected from R⁴³.

In certain embodiments a R⁴⁰ is hydroxyl optionally substituted as allowed by valence with 25 1, 2, 3, or 4 substituents selected from R⁴³.

In certain embodiments a R⁴⁰ is alkoxy optionally substituted as allowed by valence with 1, 2, 3, or 4 substituents selected from R⁴³.

In certain embodiments a R⁴⁰ is heteroaryl optionally substituted as allowed by valence with 1, 2, 3, or 4 substituents selected from R⁴³.

### Embodiments of R⁴¹ and R⁴²

In certain embodiments R⁴¹ and R⁴² are hydrogen.

In certain embodiments a R⁴¹ is hydrogen.

In certain embodiments a R⁴² is hydrogen.

In certain embodiments R⁴¹ and R⁴² are alkyl.

In certain embodiments a R⁴¹ is alkyl.

In certain embodiments a R⁴² is alkyl.

In certain embodiments R⁴¹ and R⁴² are methyl.

In certain embodiments a R⁴¹ is methyl.

In certain embodiments a R⁴² is methyl.

In certain embodiments R⁴¹ or R⁴² is haloalkyl optionally substituted as allowed by valence with 1, 2, 3, or 4 substituents selected from R⁴³.

In certain embodiments R⁴¹ or R⁴² is alkenyl or alkynyl optionally substituted as allowed by valence with 1, 2, 3, or 4 substituents selected from R⁴³.

In certain embodiments R⁴¹ or R⁴² is aryl optionally substituted as allowed by valence with 1, 2, 3, or 4 substituents selected from R⁴³.

In certain embodiments R⁴¹ or R⁴² is phenyl optionally substituted as allowed by valence with 1, 2, 3, or 4 substituents selected from R⁴³.

In certain embodiments R⁴¹ or R⁴² is heterocycle optionally substituted as allowed by valence with 1, 2, 3, or 4 substituents selected from R⁴³.

In certain embodiments R⁴¹ or R⁴² is heteroaryl optionally substituted as allowed by valence with 1, 2, 3, or 4 substituents selected from R⁴³.

In certain embodiments R⁴¹ or R⁴² is —C(O)R⁴⁰ optionally substituted as allowed by valence with 1, 2, 3, or 4 substituents selected from R⁴³.

In certain embodiments R⁴¹ or R⁴² is —S(O)R⁴⁰ optionally substituted as allowed by valence with 1, 2, 3, or 4 substituents selected from R⁴³.

In certain embodiments R⁴¹ or R⁴² is —S(O)₂R⁴⁰ optionally substituted as allowed by valence with 1, 2, 3, or 4 substituents selected from R⁴³.

### Embodiments of R⁴³

In certain embodiments a R⁴³ is halogen.

In certain embodiments a R⁴³ is cyano.

In certain embodiments a R⁴³ is nitro.

In certain embodiments a R⁴³ is alkyl.

In certain embodiments a R⁴³ is haloalkyl.

In certain embodiments a R⁴³ is alkenyl.

In certain embodiments a R⁴³ is alkynyl.

In certain embodiments a R⁴³ is aryl.

In certain embodiments a R⁴³ is heterocycle.

In certain embodiments a R⁴³ is heteroaryl.

In certain embodiments a R⁴³ is amino.

In certain embodiments a R⁴³ is hydroxyl.

In certain embodiments a R⁴³ is alkoxy.

In certain embodiments a R⁴³ is —NHalkyl.

In certain embodiments a R⁴³ is —N(alkyl)₂.

In certain embodiments a R⁴³ is —OC(O)alkyl.

In certain embodiments a R⁴³ is —NHC(O)alkyl.

In certain embodiments a R⁴³ is —N(alkyl)C(O)alkyl.

### Embodiments of R¹⁰¹

In certain embodiments a R¹⁰¹ is halogen.

In certain embodiments a R¹⁰¹ is F.

In certain embodiments a R¹⁰¹ is Cl.

In certain embodiments a R¹⁰¹ is Br.

In certain embodiments a R¹⁰¹ is alkyl.

In certain embodiments a R¹⁰¹ is methyl.

In certain embodiments a R¹⁰¹ is alkyl optionally substituted as allowed by valence with 1, 2, 3, or 4 substituents selected from R²¹.

In certain embodiments a R¹⁰¹ is haloalkyl optionally substituted as allowed by valence with 1, 2, 3, or 4 substituents selected from R²¹.

In certain embodiments a R¹⁰¹ is alkenyl optionally substituted as allowed by valence with 1, 2, 3, or 4 substituents selected from R²¹.

In certain embodiments a R¹⁰¹ is alkynyl optionally substituted as allowed by valence with 1, 2, 3, or 4 substituents selected from R²¹.

In certain embodiments a R¹⁰¹ is heterocycle optionally substituted as allowed by valence with 1, 2, 3, or 4 substituents selected from R²¹.

In certain embodiments a R¹⁰¹ is aryl optionally substituted as allowed by valence with 1, 2, 3, or 4 substituents selected from R²¹.

In certain embodiments a R¹⁰¹ is heteroaryl optionally substituted as allowed by valence with 1, 2, 3, or 4 substituents selected from R²¹.

In certain embodiments a R¹⁰¹ is cyano.

In certain embodiments a R¹⁰ is nitro.

In certain embodiments a R¹⁰¹ is —C(O)R¹⁰.

In certain embodiments a R¹⁰¹ is —OC(O)R¹⁰.

In certain embodiments a R¹⁰¹ is —NR¹¹C(O)R¹⁰.

In certain embodiments a R¹⁰¹ is —OR¹¹.

In certain embodiments a R¹⁰¹ is —NR¹¹R¹².

In certain embodiments a R¹⁰¹ is —S(O)R¹⁰.

In certain embodiments a R¹⁰¹ is —S(O)₂R¹⁰.

In certain embodiments a R¹⁰¹ is —OS(O)R¹⁰.

In certain embodiments a R¹⁰¹ is —OS(O)₂R¹⁰.

In certain embodiments a R¹⁰¹ is —NR¹¹S(O)R¹⁰.

In certain embodiments a R¹⁰¹ is —NR¹¹S(O)₂R¹⁰.

In certain embodiments a R¹⁰¹ is —SR¹¹.

### Embodiments of R²⁰⁰

In certain embodiments a R²⁰⁰ is halogen.

In certain embodiments a R²⁰⁰ is F.

In certain embodiments a R²⁰⁰ is Cl.

In certain embodiments a R²⁰⁰ is Br.

In certain embodiments a R²⁰⁰ is alkyl.

In certain embodiments a R²⁰⁰ is methyl.

In certain embodiments a R²⁰⁰ is alkyl optionally substituted as allowed by valence with 1, 2, 3, or 4 substituents selected from R²¹.

In certain embodiments a R²⁰⁰ is haloalkyl optionally substituted as allowed by valence with 1, 2, 3, or 4 substituents selected from R²¹.

In certain embodiments a R²⁰⁰ is alkenyl optionally substituted as allowed by valence with 1, 2, 3, or 4 substituents selected from R²¹.

In certain embodiments a R²⁰⁰ is alkynyl optionally substituted as allowed by valence with 1, 2, 3, or 4 substituents selected from R²¹.

In certain embodiments a R²⁰⁰ is heterocycle optionally substituted as allowed by valence with 1, 2, 3, or 4 substituents selected from R²¹.

In certain embodiments a R²⁰⁰ is aryl optionally substituted as allowed by valence with 1, 2, 3, or 4 substituents selected from R²¹.

In certain embodiments a R²⁰⁰ is heteroaryl optionally substituted as allowed by valence with 1, 2, 3, or 4 substituents selected from R²¹.

In certain embodiments a R²⁰⁰ is cyano.

In certain embodiments a R²⁰⁰ is nitro.

In certain embodiments a R²⁰⁰ is —C(O)R¹⁰.

In certain embodiments a R²⁰⁰ is —OC(O)R¹⁰.

In certain embodiments a R²⁰⁰ is —NR¹¹C(O)R¹⁰.

In certain embodiments a R²⁰⁰ is —OR¹¹.

In certain embodiments a R²⁰⁰ is —NR¹¹R¹².

In certain embodiments a R²⁰⁰ is —S(O)R¹⁰.

In certain embodiments a R²⁰⁰ is —S(O)₂R¹⁰.

In certain embodiments a R²⁰⁰ is —OS(O)R¹⁰.

In certain embodiments a R²⁰⁰ is —OS(O)₂R¹⁰.

In certain embodiments a R²⁰⁰ is —NR¹¹S(O)R¹⁰.

In certain embodiments a R²⁰⁰ is —NR¹¹S(O)₂R¹⁰.

In certain embodiments a R²⁰⁰ is —SR¹¹.

### Embodiments of “Alkyl”

In one embodiment “alkyl” is a C₁-C₁₀alkyl, C₁-C₉alkyl, C₁-C₈alkyl, C₁-C₇alkyl, C₁-C₆alkyl, C₁-C₅alkyl, C₁-C₄alkyl, C₁-C₃alkyl, or C₁-C₂alkyl.

In one embodiment “alkyl” has one carbon.

In one embodiment “alkyl” has two carbons.

In one embodiment “alkyl” has three carbons.

In one embodiment “alkyl” has four carbons.

In one embodiment “alkyl” has five carbons.

In one embodiment “alkyl” has six carbons.

Non-limiting examples of “alkyl” include: methyl, ethyl, propyl, butyl, pentyl, and hexyl.

Additional non-limiting examples of “alkyl” include: isopropyl, isobutyl, isopentyl, and isohexyl.

Additional non-limiting examples of “alkyl” include: sec-butyl, sec-pentyl, and sec-hexyl.

Additional non-limiting examples of “alkyl” include: tert-butyl, tert-pentyl, and tert-hexyl.

Additional non-limiting examples of “alkyl” include: neopentyl, 3-pentyl, and active pentyl.

In an alternative embodiment the “alkyl” group is optionally substituted.

In an alternative embodiment the “alkenyl” group is optionally substituted.

In an alternative embodiment the “alkynyl” group is optionally substituted.

### Embodiments of “Haloalkyl”

In one embodiment “haloalkyl” is a C₁-C₁₀haloalkyl, C₁-C₉haloalkyl, C₁-C₈haloalkyl, C₁-C₇haloalkyl, C₁-C₆haloalkyl, C₁-C₅haloalkyl, C₁-C₄haloalkyl, C₁-C₃haloalkyl, and C₁-C₂haloalkyl.

In one embodiment “haloalkyl” has one carbon.

In one embodiment “haloalkyl” has one carbon and one halogen.

In one embodiment “haloalkyl” has one carbon and two halogens.

In one embodiment “haloalkyl” has one carbon and three halogens.

In one embodiment “haloalkyl” has two carbons.

In one embodiment “haloalkyl” has three carbons.

In one embodiment “haloalkyl” has four carbons.

In one embodiment “haloalkyl” has five carbons.

In one embodiment “haloalkyl” has six carbons.

Non-limiting examples of “haloalkyl” include:

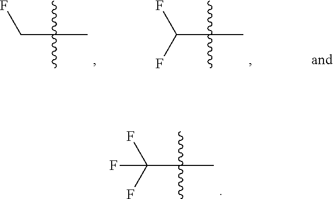

Additional non-limiting examples of “haloalkyl” include:

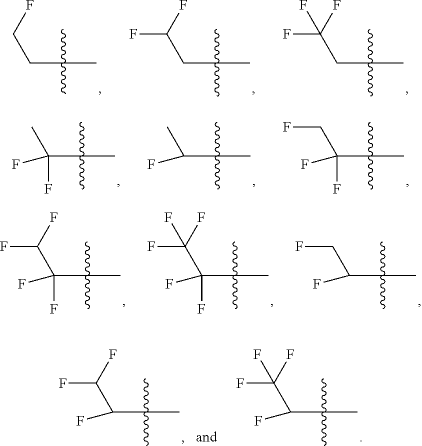

Additional non-limiting examples of “haloalkyl” include:

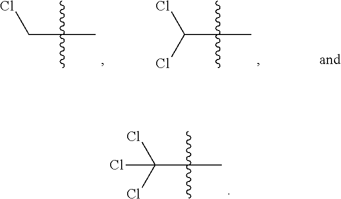

Additional non-limiting examples of “haloalkyl” include:

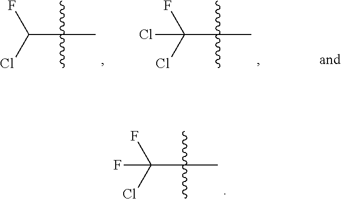

### Embodiments of “Heteroaryl”

Non-limiting examples of 5 membered “heteroaryl” groups include pyrrole, furan, thiophene, pyrazole, imidazole, triazole, isoxazole, oxazole, oxadiazole, oxatriazole, isothiazole, thiazole, thiadiazole, and thiatriazole.

Additional non-limiting examples of 5 membered “heteroaryl” groups include:

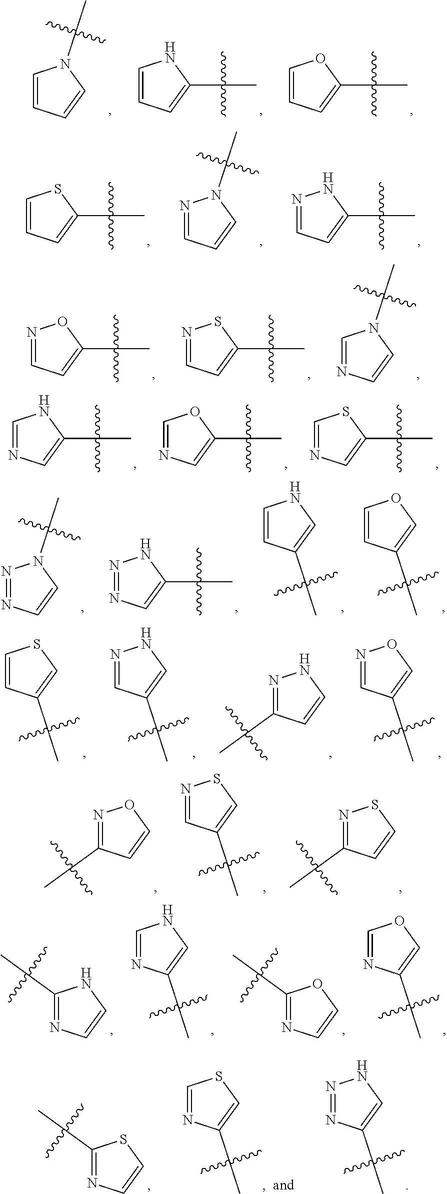

In one embodiment “heteroaryl” is a 6 membered aromatic group containing 1, 2, or 3 nitrogen atoms (i.e. pyridinyl, pyridazinyl, triazinyl, pyrimidinyl, and pyrazinyl).

Non-limiting examples of 6 membered “heteroaryl” groups with 1 or 2 nitrogen atoms include:

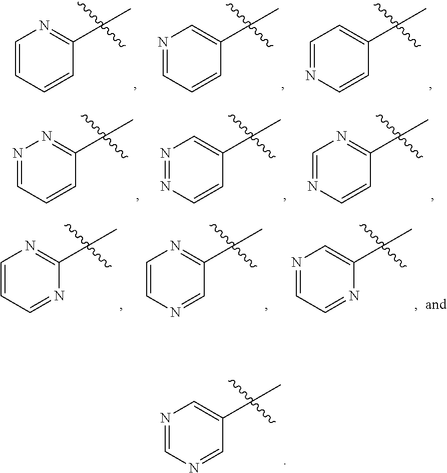

In one embodiment “heteroaryl” is a 9 membered bicyclic aromatic group containing 1 or 2 atoms selected from nitrogen, oxygen, and sulfur.

Non-limiting examples of “heteroaryl” groups that are bicyclic include indole, benzofuran, isoindole, indazole, benzimidazole, azaindole, azaindazole, purine, isobenzofuran, benzothiophene, benzoisoxazole, benzoisothiazole, benzooxazole, and benzothiazole.

Additional non-limiting examples of “heteroaryl” groups that are bicyclic include:

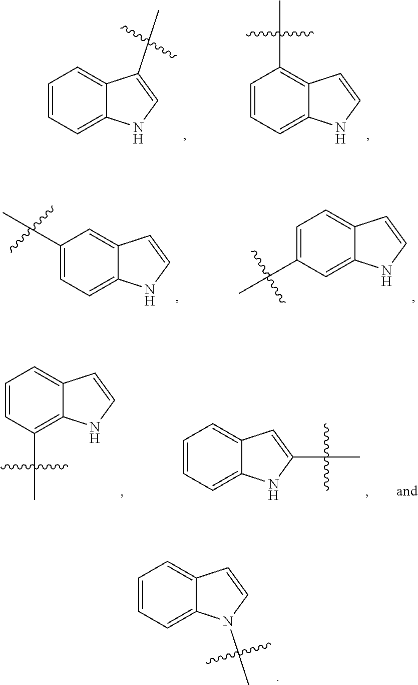

Additional non-limiting examples of “heteroaryl” groups that are bicyclic include:

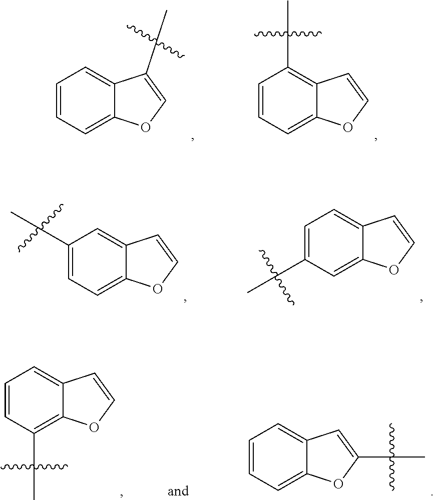 [SMILES: *c1coc2ccccc12 ; *c1cccc2occc12 ; *c1ccc2occc2c1 ; *c1ccc2ccoc2c1 ; *c1cccc2ccoc12 ; *c1cc2ccccc2o1]

Additional non-limiting examples of “heteroaryl” groups that are bicyclic include:

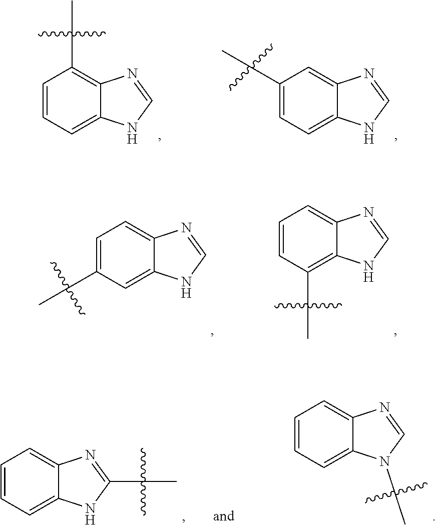 [SMILES: *c1cccc2[nH]cnc12 ; *c1ccc2[nH]cnc2c1 ; *c1ccc2nc[nH]c2c1 ; *c1cccc2nc[nH]c12 ; *c1nc2ccccc2[nH]1 ; *n1cnc2ccccc21]

In one embodiment “heteroaryl” is a 10 membered bicyclic aromatic group containing 1 or 2 atoms selected from nitrogen, oxygen, and sulfur.

Non-limiting examples of “heteroaryl” groups that are bicyclic include quinoline, isoquinoline, quinoxaline, phthalazine, quinazoline, cinnoline, and naphthyridine.

Additional non-limiting examples of “heteroaryl” groups that are bicyclic include:

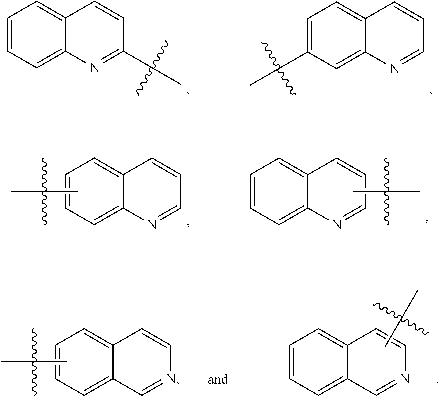

### Embodiments of “Heterocycle”

In one embodiment “heterocycle” refers to a cyclic ring with one nitrogen and 3, 4, 5, 6, 7, or 8 carbon atoms.

In one embodiment “heterocycle” refers to a cyclic ring with one nitrogen and one oxygen and 3, 4, 5, 6, 7, or 8 carbon atoms.

In one embodiment “heterocycle” refers to a cyclic ring with two nitrogens and 3, 4, 5, 6, 7, or 8 carbon atoms.

In one embodiment “heterocycle” refers to a cyclic ring with one oxygen and 3, 4, 5, 6, 7, or 8 carbon atoms.

In one embodiment “heterocycle” refers to a cyclic ring with one sulfur and 3, 4, 5, 6, 7, or 8 carbon atoms.

Non-limiting examples of “heterocycle” include aziridine, oxirane, thiirane, azetidine, 1,3-diazetidine, oxetane, and thietane.

Additional non-limiting examples of “heterocycle” include pyrrolidine, 3-pyrroline, 2-pyrroline, pyrazolidine, and imidazolidine.

Additional non-limiting examples of “heterocycle” include tetrahydrofuran, 1,3-dioxolane, tetrahydrothiophene, 1,2-oxathiolane, and 1,3-oxathiolane.

Additional non-limiting examples of “heterocycle” include piperidine, piperazine, tetrahydropyran, 1,4-dioxane, thiane, 1,3-dithiane, 1,4-dithiane, morpholine, and thiomorpholine.

Additional non-limiting examples of “heterocycle” include indoline, tetrahydroquinoline, tetrahydroisoquinoline, and dihydrobenzofuran wherein the point of attachment for each group is on the heterocyclic ring.

For example,

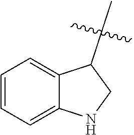 [SMILES: *C1CNc2ccccc21]

is a “heterocycle” group.

However,

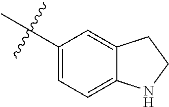 [SMILES: *c1ccc2c(c1)CCN2]

is an “aryl” group.

Non-limiting examples of “heterocycle” also include:

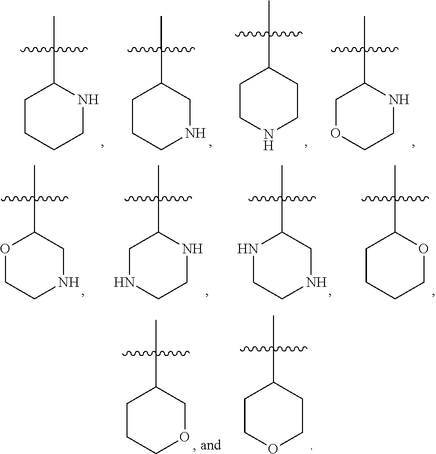

Additional non-limiting examples of “heterocycle” include:

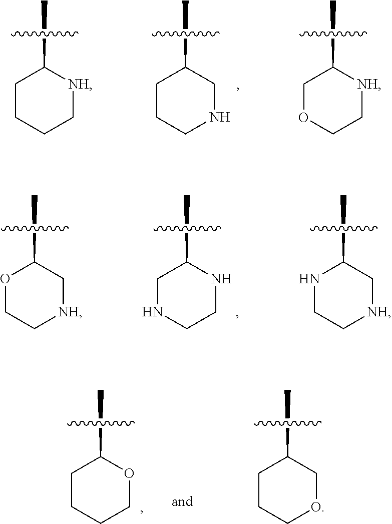

Additional non-limiting examples of “heterocycle” include:

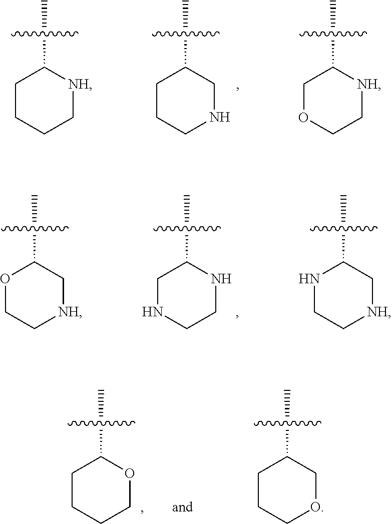

Non-limiting examples of “heterocycle” also include:

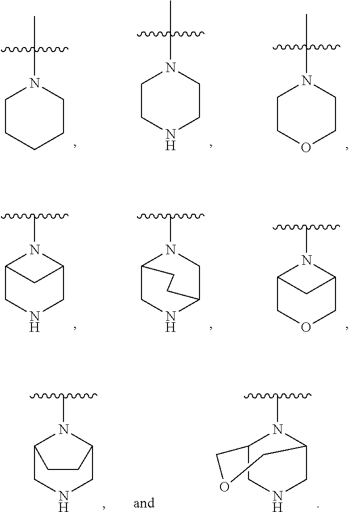

Non-limiting examples of “heterocycle” also include:

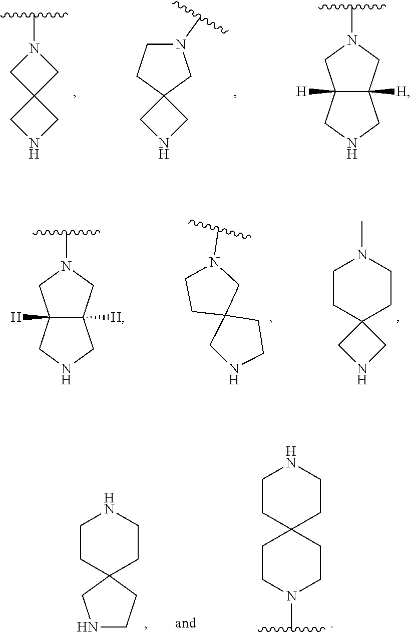

Non-limiting examples of “heterocycle” also include:

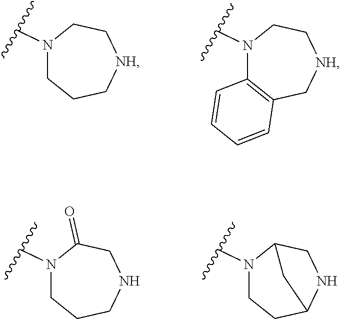 [SMILES: *N1CCCNCC1 ; *N1CCNCc2ccccc21 ; *N1CCCNCC1=O ; *N1CCC2CC1CN2]

Non-limiting examples of “heterocycle” also include:

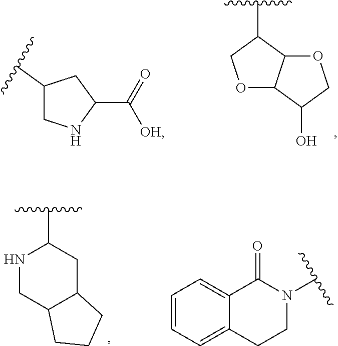 [SMILES: *C1CNC(C(=O)O)C1 ; *C1COC2C(O)COC12 ; *C1CC2CCCC2CN1 ; *N1CCc2ccccc2C1=O]

Non-limiting examples of “heterocycle” also include:

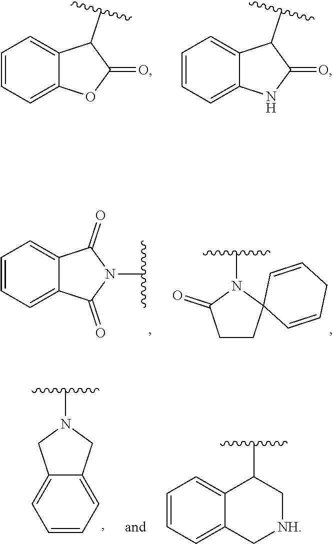

Non-limiting examples of “heterocycle” also include:

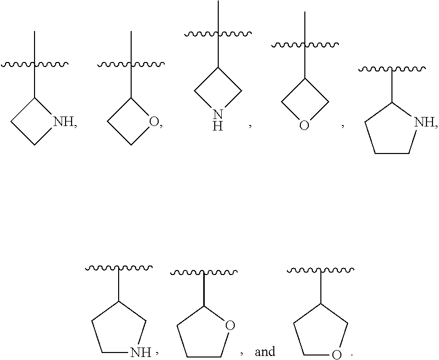

Additional non-limiting examples of “heterocycle” include:

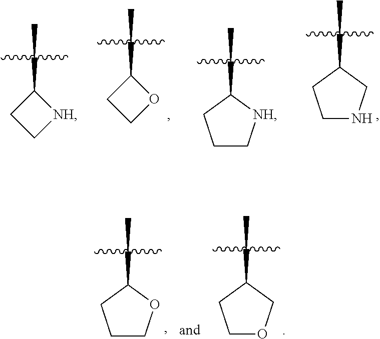 [SMILES: *[C@@H]1CCN1 ; *[C@@H]1CCO1 ; *[C@@H]1CCCN1 ; *[C@@H]1CCNC1 ; *[C@@H]1CCCO1 ; *[C@@H]1CCOC1]

Additional non-limiting examples of “heterocycle” include:

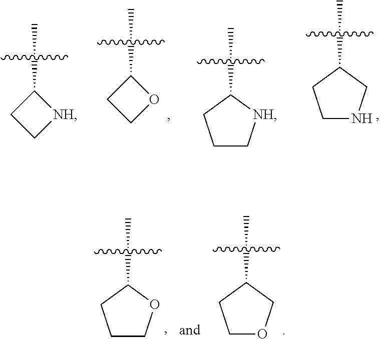 [SMILES: *[C@H]1CCN1 ; *[C@H]1CCO1 ; *[C@H]1CCCN1 ; *[C@H]1CCNC1 ; *[C@H]1CCCO1 ; *[C@H]1CCOC1]

Additional non-limiting examples of “heterocycle” include:

### Embodiments of “Aryl”

In one embodiment “aryl” is a 6 carbon aromatic group (phenyl).

In one embodiment “aryl” is a 10 carbon aromatic group (naphthyl).

In one embodiment “aryl” is a 6 carbon aromatic group fused to a heterocycle wherein the point of attachment is the aryl ring. Non-limiting examples of “aryl” include indoline, tetrahydroquinoline, tetrahydroisoquinoline, and dihydrobenzofuran wherein the point of attachment for each group is on the aromatic ring.

For example

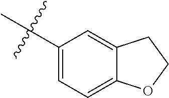 [SMILES: *c1ccc2c(c1)CCO2]

is an “aryl” group.

However,

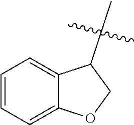 [SMILES: *C1COc2ccccc21]

is a “heterocycle” group.

### Embodiments of “Arylalkyl”

Non-limiting examples of “arylalkyl” include:

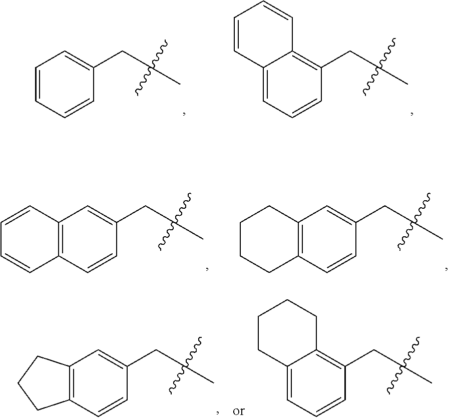

In one embodiment “arylalkyl” is

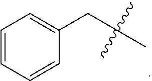 [SMILES: *Cc1ccccc1]

In one embodiment the “arylalkyl” refers to a 2 carbon alkyl group substituted with an aryl group.

Non-limiting examples of “arylalkyl” include:

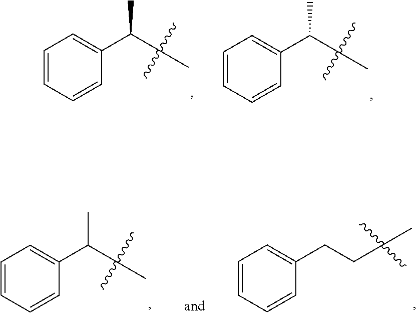

### Additional Embodiments of Formula I or Formula II

### Terminology

Compounds are described using standard nomenclature. Unless defined otherwise, all technical and scientific terms used herein have the same meaning as is commonly understood by one of skill in the art to which this invention belongs.

The protein stabilizing compounds in any of the Formulas described herein include enantiomers, mixtures of enantiomers, diastereomers, tautomers, racemates and other isomers, such as rotamers, as if each is specifically described, unless otherwise indicated or otherwise excluded by context.

The terms “a” and “an” do not denote a limitation of quantity, but rather denote the presence of at least one of the referenced item. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed. Unless defined otherwise, technical and scientific terms used herein have the same meaning as is commonly understood by one of skill in the art to which this invention belongs.

In certain embodiments the present invention includes protein stabilizing compounds with at least one desired isotopic substitution of an atom, at an amount above the natural abundance of the isotope, i.e., enriched. In certain embodiments the present invention includes protein stabilizing compounds that are not isotopically labeled.

Examples of isotopes that can be incorporated into protein stabilizing compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁷O, ¹⁸O, ¹⁸F, ³¹P, ³²P, ³⁵S, ³⁶Cl, and ¹²⁵I respectively.

In one embodiment, isotopically labelled protein stabilizing compounds can be used in metabolic studies (with, for example ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. For example, a ¹⁸F labeled protein stabilizing compound may be desirable for PET or SPECT studies. Isotopically labeled protein stabilizing compounds of this invention and prodrugs thereof can generally be prepared by carrying out the procedures disclosed in the schemes or in the examples and preparations described below by substituting a readily available isotopically labeled reagent for a non-isotopically labeled reagent.

By way of general example and without limitation, isotopes of hydrogen, for example, deuterium (²H) and tritium (³H) may optionally be used anywhere in described structures that achieves the desired result. Alternatively, or in addition, isotopes of carbon, e.g., ¹³C and ¹⁴C, may be used. In one embodiment, the isotopic substitution is replacing hydrogen with a deuterium at one or more locations on the molecule to improve the performance of the drug, for example, the pharmacodynamics, pharmacokinetics, biodistribution, half-life, stability, AUC, Tmax, Cmax, etc. For example, the deuterium can be bound to carbon in a location of bond breakage during metabolism (an α-deuterium kinetic isotope effect) or next to or near the site of bond breakage (a β-deuterium kinetic isotope effect).

Isotopic substitutions, for example deuterium substitutions, can be partial or complete. Partial deuterium substitution means that at least one hydrogen is substituted with deuterium. In certain embodiments, the isotope is 80, 85, 90, 95 or 99% or more enriched in an isotope at any location of interest. In certain embodiments deuterium is 80, 85, 90, 95 or 99% enriched at a desired location. Unless otherwise stated, the enrichment at any point is above natural abundance, and in an embodiment is enough to alter a detectable property of the drug in a human.

In one embodiment, the substitution of a hydrogen atom for a deuterium atom occurs within any variable group. For example, when any variable group is, or contain for example through substitution, methyl, ethyl, or methoxy, the alkyl residue may be deuterated (in nonlimiting embodiments, CDH₂, CD₂H, CD₃, CD₂CD₃, CHDCH₂D, CH₂CD₃, CHDCHD₂, OCDH₂, OCD₂H, or OCD₃ etc.). In certain other embodiments, a variable group has a “′” or an “a” designation, which in one embodiment can be deuterated.

The protein stabilizing compound of the present invention may form a solvate with solvents (including water). Therefore, in one embodiment, the invention includes a solvated form of the active protein stabilizing compound. The term “solvate” refers to a molecular complex of a protein stabilizing compound of the present invention (including a salt thereof) with one or more solvent molecules. Nonlimiting examples of solvents are water, ethanol, dimethyl sulfoxide, acetone and other common organic solvents. The term “hydrate” refers to a molecular complex comprising a protein stabilizing compound of the invention and water. Pharmaceutically acceptable solvates in accordance with the invention include those wherein the solvent of crystallization may be isotopically substituted, e.g. D₂O, d₆-acetone, d₆-DMSO. A solvate can be in a liquid or solid form.

A dash (“-”) that is not between two letters or symbols is used to indicate a point of attachment for a substituent. For example, —(C═O)NH₂ is attached through carbon of the keto (C═O) group.

The term “substituted”, as used herein, means that any one or more hydrogens on the designated atom or group is replaced with a moiety selected from the indicated group, provided that the designated atom's normal valence is not exceeded and the resulting protein stabilizing compound is stable. For example, when the substituent is oxo (i.e., ═O) then two hydrogens on the atom are replaced. For example a pyridyl group substituted by oxo is a pyridone. Combinations of substituents and/or variables are permissible only if such combinations result in stable protein stabilizing compounds or useful synthetic intermediates.

“Alkyl” is a branched, straight chain, or cyclic saturated aliphatic hydrocarbon group. In one embodiment, the alkyl contains from 1 to about 12 carbon atoms, more generally from 1 to about 6 carbon atoms, from 1 to about 4 carbon atoms, or from 1 to 3 carbon atoms. In one embodiment, the alkyl contains from 1 to about 8 carbon atoms. In certain embodiments, the alkyl is C₁-C₂, C₁-C₃, C₁-C₄, C₁-C₅ or C₁-C₆. The specified ranges as used herein indicate an alkyl group which is considered to explicitly disclose as individual species each member of the range described as a unique species. For example, the term C₁-C₆ alkyl as used herein indicates a straight or branched alkyl group having from 1, 2, 3, 4, 5, or 6 carbon atoms and also a carbocyclic alkyl group of 3, 4, 5, or 6 carbon atoms and is intended to mean that each of these is described as an independent species. For example, the term C₁-C₄alkyl as used herein indicates a straight or branched alkyl group having from 1, 2, 3, or 4 carbon atoms and is intended to mean that each of these is described as an independent species. When C₀-Cₙ alkyl is used herein in conjunction with another group, for example, (C₃-C₇cycloalkyl)C₀-C₄ alkyl, or —C₀-C₄alkyl(C₃-C₇cycloalkyl), the indicated group, in this case cycloalkyl, is either directly bound by a single covalent bond (C₀alkyl), or attached by an alkyl chain in this case 1, 2, 3, or 4 carbon atoms. Alkyls can also be attached via other groups such as heteroatoms as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, 2,3-dimethylbutane, and hexyl.

When a term is used that includes “alk” it should be understood that “cycloalkyl” or “carbocyclic” can be considered part of the definition, unless unambiguously excluded by the context. For example and without limitation, the terms alkyl, alkenyl, alkynyl, alkoxy, alkanoyl, alkenloxy, haloalkyl, etc. can all be considered to include the cyclic forms of alkyl, unless unambiguously excluded by context.

“Alkenyl” is a branched or straight chain aliphatic hydrocarbon group having one or more carbon-carbon double bonds that may occur at a stable point along the chain. Nonlimiting examples are C₂-C₈alkenyl, C₂-C₇alkenyl, C₂-C₆alkenyl, C₂-C₅alkenyl and C₂-C₄alkenyl. The specified ranges as used herein indicate an alkenyl group having each member of the range described as an independent species, as described above for the alkyl moiety. Examples of alkenyl include, but are not limited to, ethenyl and propenyl.

“Alkynyl” is a branched or straight chain aliphatic hydrocarbon group having one or more carbon-carbon triple bonds that may occur at any stable point along the chain, for example, C₂-C₈alkynyl or C₂-C₆alkynyl. The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

“Alkoxy” is an alkyl group as defined above covalently bound through an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Similarly an “alkylthio” or a “thioalkyl” group is an alkyl group as defined above with the indicated number of carbon atoms covalently bound through a sulfur bridge (—S—). In one embodiment, the alkoxy group is optionally substituted as described above.

“Haloalkyl” indicates both branched and straight-chain alkyl groups substituted with 1 or more halogen atoms, up to the maximum allowable number of halogen atoms. Examples of haloalkyl include, but are not limited to, trifluoromethyl, monofluoromethyl, difluoromethyl, 2-fluoroethyl, and penta-fluoroethyl.

“Aryl” indicates an aromatic group containing only carbon in the aromatic ring or rings. In one embodiment, the aryl group contains 1 to 3 separate or fused rings and is 6 to 14 or 18 ring atoms, without heteroatoms as ring members. The term “aryl” includes groups where a saturated or partially unsaturated carbocycle group is fused with an aromatic ring. The term “aryl” also includes groups where a saturated or partially unsaturated heterocycle group is fused with an aromatic ring so long as the attachment point is the aromatic ring. Such protein stabilizing compounds may include aryl rings fused to a 4 to 7 or a 5 to 7-membered saturated or partially unsaturated cyclic group that optionally contains 1, 2 or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group.

The term “heterocycle” refers to saturated and partially saturated heteroatom-containing ring radicals, where the heteroatoms may be selected from N, S, and O. The term “heterocycle” includes monocyclic 3-12 membered rings, as well as bicyclic 5-16 membered ring systems (which can include fused, bridged, or spiro, bicyclic ring systems). It does not include rings containing —O—O— or —S—S— portions. Examples of saturated heterocycle groups include saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4 to 6-membered monocyclic groups containing 1 to 2 oxygen atoms and 1 to 3 nitrogen atoms [e.g., morpholinyl]; saturated 3 to 6-membered heteromonocyclic group containing 1 to 2 sulfur atoms and 1 to 3 nitrogen atoms [e.g., thiazolidinyl]. Examples of partially saturated heterocycle radicals include but are not limited to, dihydrothienyl, dihydropyranyl, dihydrofuryl, and dihydrothiazolyl. Examples of partially saturated and saturated heterocycle groups include but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ′-benzo[d]isothiazol-6-yl, dihydropyranyl, dihydrofuryl and dihydrothiazolyl. “Bicyclic heterocycle” includes groups wherein the heterocyclic radical is fused with an aryl radical wherein the point of attachment is the heterocycle ring. “Bicyclic heterocycle” also includes heterocyclic radicals that are fused or bridged with a carbocycle radical. For example partially unsaturated condensed heterocyclic group containing 1 to 5 nitrogen atoms, for example, indoline, isoindoline, partially unsaturated condensed heterocyclic group containing 1 to 2 oxygen atoms and 1 to 3 nitrogen atoms, partially unsaturated condensed heterocyclic group containing 1 to 2 sulfur atoms and 1 to 3 nitrogen atoms, and saturated condensed heterocyclic group containing 1 to 2 oxygen or sulfur atoms.

Non-limiting examples of bicyclic heterocycles include:

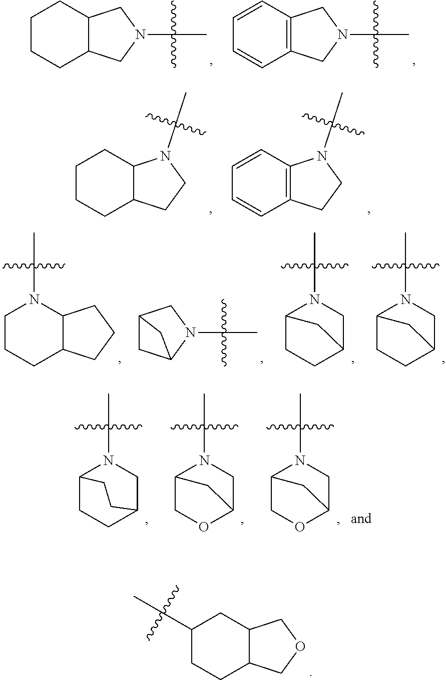

Unless otherwise drawn or clear from the context, the term “bicyclic heterocycle” includes cis and trans diastereomers. Non-limiting examples of chiral bicyclic heterocycles include:

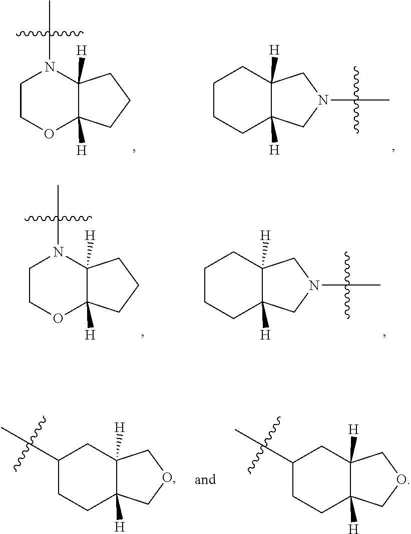 [SMILES: *N1CCO[C@H]2CCC[C@H]21 ; *N1C[C@H]2CCCC[C@H]2C1 ; *N1CCO[C@H]2CCC[C@@H]21 ; *N1C[C@@H]2CCCC[C@H]2C1 ; *C1CC[C@H]2COC[C@@H]2C1 ; *C1CC[C@H]2COC[C@H]2C1]

In certain alternative embodiments the term “heterocycle” refers to saturated and partially saturated heteroatom-containing ring radicals, where the heteroatoms may be selected from N, S, O, B, Si, and P.

The term “bicycle” refers to a ring system wherein two rings are fused together and each ring is independently selected from carbocycle, heterocycle, aryl, and heteroaryl. Non-limiting examples of bicycle groups include:

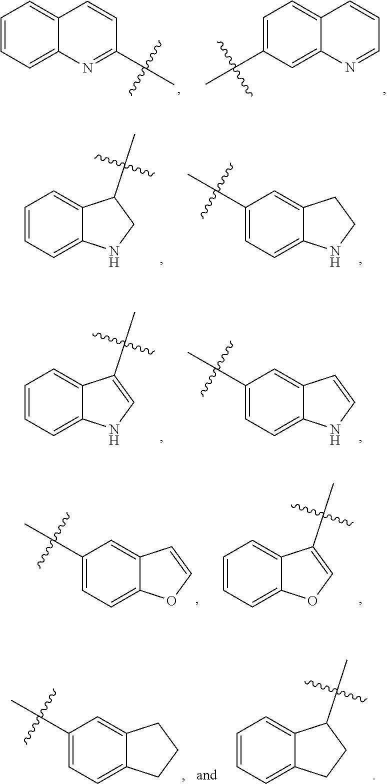

When the term “bicycle” is used in the context of a bivalent residue such as R², R³, or R⁵, the attachment points can be on separate rings or on the same ring. In certain embodiments both attachment points are on the same ring. In certain embodiments both attachment points are on different rings. Non-limiting examples of bivalent bicycle groups include:

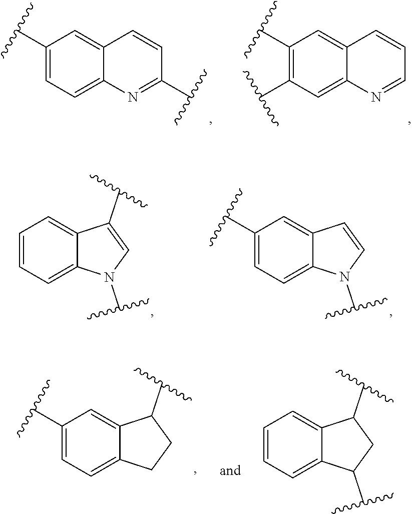 [SMILES: *c1ccc2nc(*)ccc2c1 ; *c1cc2cccnc2cc1* ; *c1cn(*)c2ccccc12 ; *c1ccc2c(ccn2*)c1 ; *c1ccc2c(c1)C(*)CC2 ; *C1CC(*)c2ccccc21]

“Heteroaryl” refers to a stable monocyclic, bicyclic, or multicyclic aromatic ring which contains from 1 to 5, or in some embodiments from 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 3, or in some embodiments from 1 to 2, heteroatoms selected from N, O, S, B or P with remaining ring atoms being carbon. In one embodiment, the only heteroatom is nitrogen. In one embodiment, the only heteroatom is oxygen. In one embodiment, the only heteroatom is sulfur. Monocyclic heteroaryl groups typically have from 5 or 6 ring atoms. In some embodiments bicyclic heteroaryl groups are 8- to 10-membered heteroaryl groups, that is, groups containing 8 or 10 ring atoms in which one 5, 6, or 7-member aromatic ring is fused to a second aromatic or non-aromatic ring wherein the point of attachment is the aromatic ring. When the total number of S and O atoms in the heteroaryl group exceeds 1, these heteroatoms are not adjacent to one another. In one embodiment, the total number of S and O atoms in the heteroaryl group is not more than 2. In another embodiment, the total number of S and O atoms in the aromatic heterocycle is not more than 1. Examples of heteroaryl groups include, but are not limited to, pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, tetrahydrofuranyl, and furopyridinyl. Heteroaryl groups are optionally substituted independently with one or more substituents described herein. “Heteroaryloxy” is a heteroaryl group as described bound to the group it substituted via an oxygen, —O—, linker.

“Heteroarylalkyl” is an alkyl group as described herein substituted with a heteroaryl group as described herein.

“Arylalkyl” is an alkyl group as described herein substituted with an aryl group as described herein.

“Heterocycloalkyl” is an alkyl group as described herein substituted with a heterocyclo group as described herein.

The term “heteroalkyl” refers to an alkyl, alkenyl, alkynyl, or haloalkyl moiety as defined herein wherein a CH₂ group is either replaced by a heteroatom or a carbon atom is substituted with a heteroatom for example, an amine, carbonyl, carboxy, oxo, thio, phosphate, phosphonate, nitrogen, phosphorus, silicon, or boron. In one embodiment, the only heteroatom is nitrogen. In one embodiment, the only heteroatom is oxygen. In one embodiment, the only heteroatom is sulfur. In one embodiment, “heteroalkyl” is used to indicate a heteroaliphatic group (cyclic, acyclic, substituted, unsubstituted, branched or unbranched) having 1-20 carbon atoms. Nonlimiting examples of heteroalkyl moieties include polyethylene glycol, polyalkylene glycol, amide, polyamide, polylactide, polyglycolide, thioether, ether, alkyl-heterocycle-alkyl, —O-alkyl-O-alkyl, alkyl-O-haloalkyl, etc.

A “dosage form” means a unit of administration of an active agent. Examples of dosage forms include tablets, capsules, injections, suspensions, liquids, emulsions, implants, particles, spheres, creams, ointments, suppositories, inhalable forms, transdermal forms, buccal, sublingual, topical, gel, mucosal, and the like. A “dosage form” can also include an implant, for example an optical implant.

“Pharmaceutical compositions” are compositions comprising at least one active agent, and at least one other substance, such as a carrier. The present invention includes pharmaceutical compositions of the described compounds.

“Pharmaceutical combinations” are combinations of at least two active agents which may be combined in a single dosage form or provided together in separate dosage forms with instructions that the active agents are to be used together to treat any disorder described herein.

A “pharmaceutically acceptable salt” is a derivative of the disclosed protein stabilizing compound in which the parent protein stabilizing compound is modified by making inorganic and organic, pharmaceutically acceptable, acid or base addition salts thereof. The salts of the present protein stabilizing compounds can be synthesized from a parent protein stabilizing compound that contains a basic or acidic moiety by conventional chemical methods. Generally, such salts can be prepared by reacting free acid forms of these protein stabilizing compounds with a stoichiometric amount of the appropriate base (such as Na, Ca, Mg, or K hydroxide, carbonate, bicarbonate, or the like), or by reacting free base forms of these protein stabilizing compounds with a stoichiometric amount of the appropriate acid. Such reactions are typically carried out in water or in an organic solvent, or in a mixture of the two. Salts of the present protein stabilizing compounds further include solvates of the protein stabilizing compounds and of the protein stabilizing compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent protein stabilizing compound formed, for example, from inorganic or organic acids. Examples, of such salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

The term “carrier” applied to pharmaceutical compositions/combinations of the invention refers to a diluent, excipient, or vehicle with which an active protein stabilizing compound is provided.

A “pharmaceutically acceptable excipient” means an excipient that is useful in preparing a pharmaceutical composition/combination that is generally safe, acceptable for human consumption, and neither biologically nor otherwise inappropriate for administration to a host, typically a human. In one embodiment, an excipient is used that is acceptable for veterinary use.

A “patient” or “host” or “subject” is a human or non-human animal in need of treatment or prevention of any of the disorders as specifically described herein. Typically, the host is a human. A “patient” or “host” or “subject” also refers to for example, a mammal, primate (e.g., human), cow, sheep, goat, horse, dog, cat, rabbit, rat, mice, bird and the like.

A “therapeutically effective amount” of a compound, pharmaceutical composition, or combination of this invention means an amount effective, when administered to a host, provides a therapeutic benefit such as an amelioration of symptoms or reduction or diminution of the disease itself.

### Pharmaceutical Compositions

A protein stabilizing compound of the present invention or a pharmaceutically acceptable salt, solvate or prodrug thereof as disclosed herein can be administered as a neat chemical, but is more typically administered as a pharmaceutical composition that includes an effective amount for a host, typically a human, in need of such treatment to treat a disorder mediated by the Target Ubiquitinated Protein, as described herein or otherwise well-known for that Target Ubiquitinated Protein.

A protein stabilizing compound of the present invention can be administered in any manner that allows the protein stabilizing compound to stabilize the Target Ubiquitinated Protein. As such, examples of methods to deliver a protein stabilizing compound of the present invention include, but are not limited to, oral, intravenous, sublingual, subcutaneous, parenteral, buccal, rectal, intra-aortal, intracranial, subdermal, transdermal, controlled drug delivery, intramuscular, or transnasal, or by other means, in dosage unit formulations containing one or more conventional pharmaceutically acceptable carriers, as appropriate. In certain embodiments, a protein stabilizing compound of the present invention is provided in a liquid dosage form, a solid dosage form, a gel, particle, etc.

In certain embodiments the protein stabilizing compound of the present invention is administered subcutaneously. Typically, the protein stabilizing compound will be formulated in a liquid dosage form for subcutaneous injection, such as a buffered solution. Non-limiting examples of solutions for subcutaneous injection include phosphate buffered solution and saline buffered solution. In certain embodiments the solution is buffered with multiple salts.

In certain embodiments the protein stabilizing compound of the present invention is administered intravenously. Typically, if administered intravenously, the protein stabilizing compound will be formulated in a liquid dosage form for intravenous injection, such as a buffered solution. Non-limiting examples of solutions for intravenous injection include phosphate buffered solution and saline buffered solution. In certain embodiments the solution is buffered with multiple salts.

Therefore, the disclosure provides pharmaceutical compositions comprising an effective amount of protein stabilizing compound or its pharmaceutically acceptable salt together with at least one pharmaceutically acceptable carrier for any appropriate use thereof. The pharmaceutical composition may contain a protein stabilizing compound or salt as the only active agent, or, in an alternative embodiment, the protein stabilizing compound and at least one additional active agent.

The term “pharmaceutically acceptable salt” as used herein refers to a salt of the described protein stabilizing compound which is, within the scope of sound medical judgment, suitable for administration to a host such as a human without undue toxicity, irritation, allergic response, and the like, commensurate with a reasonable benefit/risk ratio, and effective for its intended use. Thus, the term “pharmaceutically acceptable salt” refers to the relatively non-toxic, inorganic and organic acid addition salts of the presently disclosed protein stabilizing compounds. These salts can be prepared during the final isolation and purification of the protein stabilizing compounds or by separately reacting the purified protein stabilizing compound in its free form with a suitable organic or inorganic acid and then isolating the salt thus formed. Basic protein stabilizing compounds are capable of forming a wide variety of different salts with various inorganic and organic acids. Acid addition salts of the basic protein stabilizing compounds are prepared by contacting the free base form with a sufficient amount of the desired acid to produce the salt in the conventional manner. The free base form can be regenerated by contacting the salt form with a base and isolating the free base in the conventional manner. The free base forms may differ from their respective salt forms in certain physical properties such as solubility in polar solvents. Pharmaceutically acceptable base addition salts may be formed with a metal or amine, such as alkali and alkaline earth metal hydroxide, or an organic amine. Examples of metals used as cations, include, but are not limited to, sodium, potassium, magnesium, calcium, and the like. Examples of suitable amines include, but are not limited to, N,N′-dibenzylethylenediamine, chloroprocaine, choline, diethanolamine, ethylenediamine, N-methylglucamine, and procaine. The base addition salts of acidic protein stabilizing compounds are prepared by contacting the free acid form with a sufficient amount of the desired base to produce the salt in the conventional manner. The free acid form can be regenerated by contacting the salt form with an acid and isolating the free acid in a conventional manner. The free acid forms may differ from their respective salt forms somewhat in certain physical properties such as solubility in polar solvents.

Salts can be prepared from inorganic acids sulfate, pyrosulfate, bisulfate, sulfite, bisulfite, nitrate, phosphate, monohydrogenphosphate, dihydrogenphosphate, metaphosphate, pyrophosphate, chloride, bromide, iodide, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts can also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts can include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. See, for example, Berge et al., J. Pharm. Sci., 1977, 66, 1-19, which is incorporated herein by reference.

Any dosage form can be used that achieves the desired results. In certain embodiments the pharmaceutical composition is in a dosage form that contains from about 0.1 mg to about 1500 mg, from about 10 mg to about 1000 mg, from about 100 mg to about 800 mg, or from about 200 mg to about 600 mg of the active protein stabilizing compound and optionally from about 0.1 mg to about 1500 mg, from about 10 mg to about 1000 mg, from about 100 mg to about 800 mg, or from about 200 mg to about 600 mg of an additional active agent in a unit dosage form. Examples are dosage forms with at least 0.1, 1, 5, 10, 25, 50, 100, 200, 250, 300, 400, 500, 600, 700, or 750 mg of active protein stabilizing compound, or its salt.

In certain embodiments the dose ranges from about 0.01-100 mg/kg of patient bodyweight, for example about 0.01 mg/kg, about 0.05 mg/kg, about 0.1 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg.

In some embodiments, a protein stabilizing compound disclosed herein or used as described is administered once a day (QD), twice a day (BID), or three times a day (TID). In some embodiments, a protein stabilizing compound disclosed herein or used as described is administered at least once a day for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 35 days, at least 45 days, at least 60 days, at least 75 days, at least 90 days, at least 120 days, at least 150 days, at least 180 days, or longer.

In certain embodiments the protein stabilizing compound of the present invention is administered once a day, twice a day, three times a day, or four times a day.

The pharmaceutical composition may be formulated as any pharmaceutically useful form, e.g., a pill, capsule, tablet, an injection or infusion solution, a syrup, an inhalation formulation, a suppository, a buccal or sublingual formulation, a parenteral formulation, or in a medical device. Some dosage forms, such as tablets and capsules, can be subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the protein stabilizing compound is sufficient to provide a practical quantity of material for administration per unit dose of the protein stabilizing compound. If provided as in a liquid, it can be a solution or a suspension.

Representative carriers include phosphate buffered saline, water, solvent(s), diluents, pH modifying agents, preservatives, antioxidants, suspending agents, wetting agent, viscosity agents, tonicity agents, stabilizing agents, and combinations thereof. In some embodiments, the carrier is an aqueous carrier. Examples of aqueous carries include, but are not limited to, an aqueous solution or suspension, such as saline, plasma, bone marrow aspirate, buffers, such as Hank's Buffered Salt Solution (HBSS), HEPES (4-(2-hydroxyethyl)-1-piperazineethanesulfonic acid), Ringers buffer, ProVisc®, diluted ProVisc®, Provisc® diluted with PBS, Krebs buffer, Dulbecco's PBS, normal PBS, sodium hyaluronate solution, citrate buffer, simulated body fluids, plasma platelet concentrate and tissue culture medium or an aqueous solution or suspension comprising an organic solvent. Acceptable solutions include, for example, water, Ringer's solution and isotonic sodium chloride solutions. The formulation may also be a sterile solution, suspension, or emulsion in a non-toxic diluent or solvent such as 1,3-butanediol.

Viscosity agents may be added to the pharmaceutical composition to increase the viscosity of the composition as desired. Examples of useful viscosity agents include, but are not limited to, hyaluronic acid, sodium hyaluronate, carbomers, polyacrylic acid, cellulosic derivatives, polycarbophil, polyvinylpyrrolidone, gelatin, dextin, polysaccharides, polyacrylamide, polyvinyl alcohol (including partially hydrolyzed polyvinyl acetate), polyvinyl acetate, derivatives thereof and mixtures thereof.

Solutions, suspensions, or emulsions for administration may be buffered with an effective amount necessary to maintain a pH suitable for the selected administration. Suitable buffers are well known by those skilled in the art. Some examples of useful buffers are acetate, borate, carbonate, citrate, and phosphate buffers. Solutions, suspensions, or emulsions for topical, for example, ocular administration may also contain one or more tonicity agents to adjust the isotonic range of the formulation. Suitable tonicity agents are well known in the art. Some examples include glycerin, mannitol, sorbitol, sodium chloride, and other electrolytes.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the protein stabilizing compound of the present invention.

The pharmaceutical compositions/combinations can be formulated for oral administration. These compositions can contain any amount of active protein stabilizing compound that achieves the desired result, for example between 0.1 and 99 weight % (wt. %) of the protein stabilizing compound and usually at least about 1 wt. % of the protein stabilizing compound. Some embodiments contain from about 25 wt. % to about 50 wt. % or from about 5 wt. % to about 75 wt. % of the protein stabilizing compound. Enteric coated oral tablets may also be used to enhance bioavailability of the protein stabilizing compound for an oral route of administration.

Formulations suitable for rectal administration are typically presented as unit dose suppositories. These may be prepared by admixing the active protein stabilizing compound with one or more conventional solid carriers, for example, cocoa butter, and then shaping the resulting mixture.

### Target Ubiquitinated Protein and Ubiquitinated Protein Targeting Ligands

The compounds described herein include a Ubiquitinated Protein Targeting Ligand. In certain embodiments, the Ubiquitinated Protein Targeting Ligand is a small organic molecule (e.g. not an inorganic substance or peptide) that binds to the Target Ubiquitinated Protein adequately to facilitate deubiquitination. In certain embodiments of the invention, the Ubiquitinated Protein Targeting Ligand is a is a peptide or oligonucleotide that binds to the Target Ubiquitinated Protein adequately to facilitate deubiquitination. In certain embodiments the Ubiquitinated Protein Targeting Ligand is a pharmaceutically active compound or a fragment thereof that binds to the Target Ubiquitinated Protein (for example an approved drug or a compound in development with known binding affinity for the Target Ubiquitinated Protein in either the ubiquitinated or nonubiquitinated form). A plethora of illustrative nonlimiting examples or Ubiquitinated Protein Targeting Ligands for use in the present invention are provided in the Detailed Description and Figures. Additional Ubiquitinated Protein Targeting Ligand are known in the art.

Where proteins are referred to by their abbreviations both wild type and non-wild type versions of the protein are contemplated unless excluded by context. For example, where the Target Ubiquitinated Protein is CFTR the CFTR may be wild-type or have one or more mutations.

In certain embodiments the Ubiquitinated Protein Targeting Ligand binds the Target Ubiquitinated Protein before it is ubiquitinated and prevents ubiquitination or removes ubiquitins that are added subsequently. In other embodiments the Ubiquitinated Protein Targeting Ligand binds the Target Ubiquitinated Protein after it is ubiquitinated and prevents further ubiquitination or removes ubiquitins that are added subsequently.

In certain embodiments the Target Ubiquitinated Protein is a mediator of a renal disease, for example CLDN16, CLDN19, FXYD2, UMOD, SLC12A3, SLC4A1, SCNN1B, SCNN1G, AVPR2, AQP2, CFTR, GLA, COL4A3, COL4A4, COL4A5, COL4A1, ACTN4, TRPC6, INF2, MYO1E, NPHS1, NPHS2, LAMB2, CTNS, SLC3A1, CLCN5, OCRL, SLC34A3, PHEX, FGF23, DMP1, OCRL, SLC4A4, SLC5A2, SLC5A1, SLC12A1, KCNJ1, BSND.

Non-limiting examples of renal disease include hypomagnesaemia type 2, hypomagnesaemia type 3, hypomagnesaemia type 5, uromodulin-associated kidney disease, gitelman syndrome, distal renal tubular acidosis, Liddle syndrome, nephrogenic diabetes insipidus, cystic fibrosis, Fabry disease, Alport syndrome, hereditary angiopathy with nephropathy aneurysms and muscle cramps (HANAC), focal segmental glomerulosclerosis 1, focal segmental glomerulosclerosis 2, focal segmental glomerulosclerosis 5, focal segmental glomerulosclerosis 6, nephrotic syndrome type 1, nephrotic syndrome type 2, Pierson syndrome, cystinosis, cystinuria type A, Dent's disease 1, Dent's disease 2, hypophosphataemic rickets with hypercalciuria, hypophosphataemic rickets, Lowe syndrome, proximal renal tubular acidosis, renal glucosuria, Bartter syndrome antenatal type 1, Bartter syndrome antenatal type 2, Bartter syndrome type 4,

As used herein 4-character identifier referring to crystal structures are RCS Protein Data Base (PDB) crystal structure identifiers and 3-character identifiers referring to ligands are PDB ligand identifiers. The skilled artisan will recognize that these codes can be entered into the PDB to view crystal structures of the referenced proteins and ligands. These crystal structures provide direction for where to attach the linker to the targeting ligand while maintaining binding efficacy. For example 602P refers to a crystal structure of cystic fibrosis transmembrane conductance regulator protein (CFTR) in complex with ivacaftor. By entering 602P into the PDB (for example at https://www.rcsb.org/) the crystal structure can be viewed.

### CFTR

In certain embodiments the protein stabilizing compound of the present invention includes a CFTR targeting ligand and can be used in the treatment of a CFTR mediated disease such as cystic fibrosis, male infertility, polycystic kidney disease, obstructive lung disease, intestinal obstruction syndromes, liver dysfunction, exocrine and endocrine pancreatic dysfunction, or secretory diarrhea.

CFTR is a glycoprotein with 1480 amino acids and is classified as an ABC (ATP-binding cassette) transporter. The cystic fibrosis transmembrane conductance regulator protein (CFTR) is a cAMP activated chloride ion (Cr) channel responsible for Cl— transport. CFTR is expressed in epithelial cells in mammalian airways, intestine, pancreas and testis. It is there where CFTR provides a pathway for the movement of Cl— ions across the apical membrane and a key point at which to regulate the rate of transepithelial salt and water transport. Hormones, such as a 0-adrenergic agonist, or toxins, such as cholera toxin, lead to an increase in cAMP, activation of cAMP-dependent protein kinase, and phosphorylation of the CFTR Cl— channel, which causes the channel to open. An increase in the concentration of Ca2+ in a cell can also activate different apical membrane channels. Phosphorylation by protein kinase C can either open or shut Cl— channels in the apical membrane.

The CFTR protein consists of five domains. There are two nucleotide binding domains (NBD1 and NBD2), regulatory domain (RD) and two transmembrane domains (TMD1 and TMD2). The protein activity is regulated by cAMP-dependent Protein Kinase (PKA) which catalyze phosphorylation of regulatory domain (RD) and also binding of two ATP molecules to NBD1 and NBD2 domains. Nonlimiting examples of CFTR mutant proteins include ΔF508 CFTR, G551D-CFTR, G1349D-CFTR, D1152H-CFTR, E56K, P67L, E92K, L206W. These mutations cause CFTR to be dysfunctional (e.g. operate with less activity that WT CFTR).

Dysfunction of CFTR is associated with a wide spectrum of disease, including cystic fibrosis (CF) and with some forms of male infertility, polycystic kidney disease, obstructive lung disease, intestinal obstruction syndromes, liver dysfunction, exocrine and endocrine pancreatic dysfunction and secretory diarrhea. CF is a hereditary disease that mainly affects the lungs and digestive system, causing progressive disability and early death. With an average life expectancy of around 31 years, CF is one of the most common life-shortening, childhood-onset inherited diseases. This disease is caused by mutation of the gene encoding CFTR, and is autosomal recessive.

In certain embodiments, the Ubiquitinated Protein Targeting Ligand is a ligand for CFTR selected from a small molecule, polypeptide, peptidomimetic, antibody, antibody fragment, antibody-like protein, and nucleic acid. In some embodiments, the CFTR Targeting Ligand is a corrector agent (e.g., a ligand that activates CFTR or rescues CFTR or mutant CFTR from degradation).

In certain embodiments, CFTR correctors are molecules that correct one or more defects by rescuing proteins from endoplasmic reticulum degradation, improving trafficking of CFTR to the cell surface, and/or inhibiting proteins that are involved in the recycling of CFTR in the cell membrane. Several correctors have been identified using high throughput assays (O'Sullivan & Freedman (2009) Lancet 373:1991-2004).

In certain embodiments, CFTR corrector compound is selected from corr-4a (Pedemonte, et al. (2005) J. Clin. Invest. 115:2564) and Lumacaftor (VX-809), which partially alleviate the folding defect and allows some ΔF508-CFTR to reach the apical membrane (Van Goor, et al. (2009) Pediatr. Pulmonol. 44:S154-S155; Van Goor, et al. (2011) Proc. Natl. Acad. Sci. USA 108:18843-18848).

In certain embodiments the CFTR Targeting Ligand is a compound described in WO2016077413A1, WO2010048125A2, or WO2013070529A1.

In certain embodiments the CFTR Targeting Ligand is a polypeptide. In certain embodiments the polypeptide is at least about 3, 5, 10, 15, 20, 25, 30, 35, 40, 45, 50, 55, 60, 65, 70, 75, 80, 90, 100, 110, 120, 130, 140, 150, 175, 200, 225 or 250 amino acids in length. In certain embodiments, the polypeptide is about 5-10, 5-25, 5-50, 5-75, 5-100, 5-150 or 5-200 amino acids in length. In certain embodiments, the polypeptide is membrane permeable.

In certain embodiments, the CFTR Targeting Ligand comprises a chimeric polypeptide which further comprises one or more fusion domains. Nonlimiting examples of chimeric polypeptides comprising one or more fusion domains include polyhistidine, Glu-Glu, glutathione S transferase (GST), thioredoxin, protein A, protein G, and an immunoglobulin heavy chain constant region (Fc), maltose binding protein (MBP).

In certain embodiments, the CFTR Targeting Ligand comprises a chimeric polypeptide comprising a first portion that is a polypeptide corrector agent, and a second portion that serves as a targeting moiety. In certain embodiments, the targeting moiety targets a subject's lungs, pancreas, liver, intestines, sinuses, and/or sex organs.

In certain embodiments, the CFTR Targeting Ligand may further comprise post-translational modifications. Exemplary post-translational protein modifications include phosphorylation, acetylation, methylation, ADP-ribosylation, ubiquitination, glycosylation, carbonylation, sumoylation, biotinylation or addition of a polypeptide side chain or of a hydrophobic group. As a result, the CFTR Targeting Ligand may contain non-amino acid elements, such as lipids, poly- or mono-saccharide, and phosphates.

In certain embodiments, the CFTR Targeting Ligand is a potentiator which enhances the activity of CFTR that is correctly located at the cell membrane. CFTR potentiators are particularly useful in the treatment of subjects with class III mutations.

Non-limiting examples of CFTR potentiators include, but are not limited to, certain flavones and isoflavones, such as genistein, which are capable of stimulating CFTR-mediated chloride transport in epithelial tissues in a cyclic-AMP independent manner (See U.S. Pat. No. 6,329,422, incorporated herein by reference in its entirety); phenylglycine-01 (2-[(2-1H-indol-3-yl-acetyl)-methylamino]-N-(4-isopropylphenyl)-2-phenylacetamide); felodipine (Ethylmethyl-4-(2,3-dichlorophenyl)-2,6-dimethyl-1,4-dihydro-3, 5-pyridinedicarboxylate); sulfonamide SF-01 (6-(ethylphenylsulfamoyl)-4-oxo-1,4-dihydroquinoline-3-carboxylic acid cycloheptylamide); UCCF-152 (3-[2-(benzyloxy) phenyl]-5-(chloromethyl) isoxazole), and Ivacaftor (VX-770; N—(2,-Di-tert-butyl-5-hydroxyphenyl)-4-oxo-1, 4-dihydroquinoline-3-carboxamide).

In certain embodiments, the compounds described herein is used in addition to a dual corrector and potentiator activities. In certain embodiments, non-limiting examples of dual correctors and potentiators include VRT-532 (3-(2-hydroxy-5-methylphenyl)-5-phenylpyrazole) and cyanoquinolines such as N—(2-((3-Cyano-5,7-dimethylquinolin-2-yl) amino) ethyl)-3-methoxybenzamide (CoPo-2), hybrid bithiazole-phenylglycine corrector-potentiators which, when cleaved by intestinal enzymes, yield an active bithiazole corrector and phenylglycine potentiator (Mills, et al. (2010) Bioorg. Med. Chem. Lett. 20:87-91). The only FDA-approved CFTR activator, VX-770, is a “potentiator” developed by the treatment of CF by correcting the channel gating of certain CFTR mutations.

A compound described herein with a CFTR Targeting Ligand removes ubiquitin from Ubiquitinated CFTR in a manner that stabilizes CFTR and in some embodiments restore the CFTR's function. For example, when the Target Ubiquitinated CFTR has a mutation that causes it to incorrectly fold, a compound of the present invention with a CFTR Targeting Ligand that is a corrector may increase its activity by removing ubiquitins and correcting its folding so that it may function correctly. When the Target Ubiquitinated CFTR has a mutation that causes it to less effectively function as a gating and conduction protein, a compound of the present invention with a CFTR Targeting Ligand that is a potentiator may increase its activity by removing ubiquitins and potentiating the protein.

In certain embodiments a compound of the present invention with a CFTR Targeting Ligand or a pharmaceutically acceptable salt thereof is used in combination with a potentiator of CFTR or a pharmaceutically acceptable salt thereof to treat cystic fibrosis. In certain embodiments a compound of the present invention with a CFTR Targeting Ligand or a pharmaceutically acceptable salt thereof is used in combination with a corrector of CFTR or a pharmaceutically acceptable salt thereof to treat cystic fibrosis. Non-limiting examples of CFTR potentiators include ivacaftor, deutivacaftor, and ABBV-974. Non-limiting examples of CFTR correctors include lumacaftor, tezacaftor, posenacaftor, olacaftor, bamocaftor, and elexacaftor. In certain embodiments a compound of the present invention has a CFTR Targeting Ligand that is a potentiator and the compound is used in combination with a CFTR corrector. In certain embodiments a compound of the present invention has a CFTR Targeting Ligand that is a corrector and the compound is used in combination with a CFTR potentiator.

In certain embodiments, the CFTR Targeting Ligand is selected from Ataluren (3˜[5-(2-Fluorophenyl)-1, 2, 4-oxadiazol-3-yl]benzoic acid), Lumacaftor (VX-809; 3-{6-{[1-(2, 2-difluoro-1, 3-benzodioxol-5-yl) cyclopropanecarbonyl]amino}-3-methylpyridin-2-yl}benzoic acid), ivacaftor, VX-661, FDL169, N91115, QBW251, Riociguat, QR-010, lumacaftor, GLPG222, VX-152, VX-440, VX-445, VX-561 (aka CTP-656), VX-659, PTI-428, PTI-801, and PTI-808.

In certain embodiments a compound described herein stabilizes wildtype CFTR and/or mutant CFTR that has been ubiquitinated and thus tagged for proteasomal degradation and removes enough ubiquitins to allow the compound to be trafficked back to the cell membrane and thus restore function.

In certain embodiments the protein stabilizing compound contains lumacaftor or a derivative or fragment thereof:

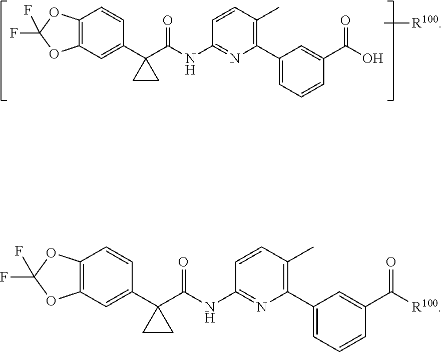

In certain embodiments the protein stabilizing compound contains ivacaftor or a derivative or fragment thereof:

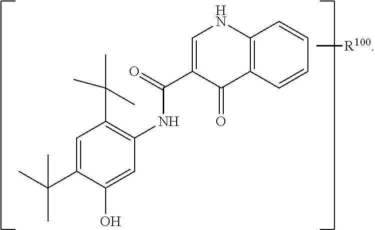

In certain embodiments the protein stabilizing compound contains tezacaftor or a derivative or fragment thereof:

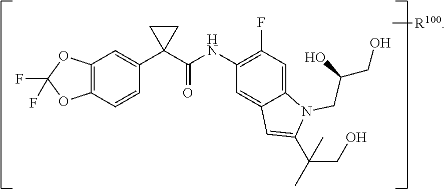

### PAH

In certain embodiments the protein stabilizing compound of the present invention includes a PAH targeting ligand and can be used in the treatment of a PAH-mediated disease such as PAH deficiency (e.g. phenylketonuria (PKU), non-PKU hyperphenylalaninemia (HPA), or variant PKU).

Phenylalanine hydroxylase (PAH) catalyzes the hydroxylation of phenylalanine to tyrosine. It exists as an equilibrium of monomeric and dimeric forms (monomer size 51.9 kDa) and contains a catalytic nonheme iron in the catalytic site. The hydroxylation proceeds through an iron (IV) oxo intermediate generated by the tetrahydrobiopterin cofactor. Although phenylalanine is utilized in protein synthesis, most of the dietary phenylalanine is broken down into carbon dioxide and water over a series of steps. The rate limiting step in phenylalanine catabolism is hydroxylation to tyrosine, which provides a synthetic handle for later enzymes to break down the aromatic side chain. Deficiencies in PAH are inherited in an autosomal recessive manner, and lead to a dangerous buildup of phenylalanine causing seizures, intellectual disability, and microcephaly in infected children. Preventing symptomatic PKU requires strict adherence to a physician prescribed diet to reduce the intake of the amino acid phenylalanine. Additional supplementation with tyrosine and other downstream metabolites is required for proper development.

Non-limiting examples of crystal structures of PAH with Protein Recognition Moieties include 4JPY, 1LTZ, 4ANP, 1KW0, 1TG2, 3PAH, 4PAH, 5PAH, 6PAH, and 5JK5.

In certain embodiments the PAH Targeting Ligand is selected from

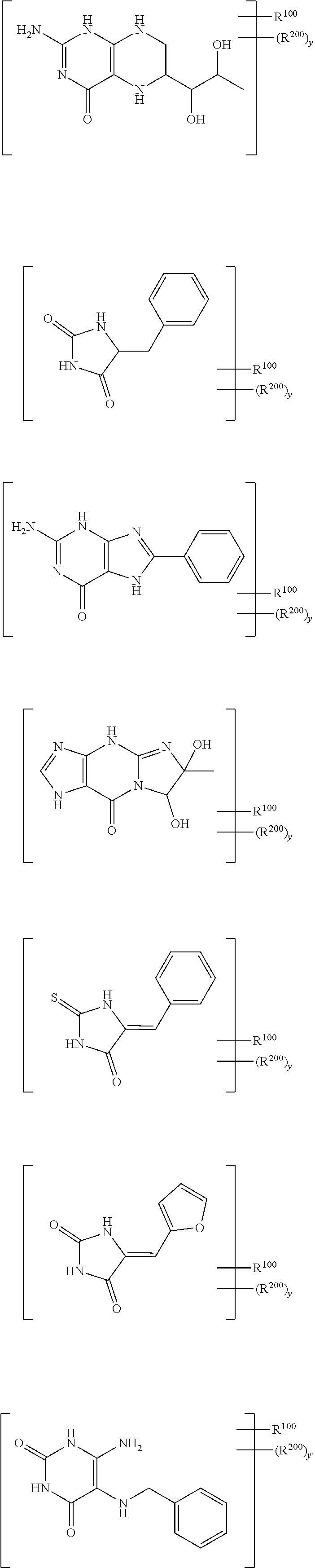

### ABCA4

In certain embodiments the protein stabilizing compound of the present invention includes a ABCA4 Targeting Ligand and can be used in the treatment of a ABCA4-mediated disease such as Stargardt disease or retinal degeneration.

ATP-binding cassette, sub family A, member 4 (ABCA4) is a transporter protein expressed in rod photoreceptors of the eye. The protein consists of two extracellular domains, two intracellular domains, and two transmembrane domains. Upon binding of ATP to the intracellular nucleotide binding site, the transmembrane domain changes shape to facilitate transport of retinoid ligands. As retinoids degrade, they form covalent adducts with phosphatidoethanolamine which generates a charged species that is recognized by ABCA4. In knockout mice, photobleaching the retina with strong light causes a significant buildup of the N-retinyl-phosphatidylethanolamine. Toxic levels of this molecule cause age-related macular degeneration. In humans, mutations of ABCA4 lead to Stargardt macular dystrophy, a juvenile macular degeneration in which the photoreceptors of the macula die off causing central blindness.

In certain embodiments the protein stabilizing compound contains lumacaftor or a derivative or fragment thereof and can be used for the treatment of an ABCA4-mediated disorder such as Stargardt disease:

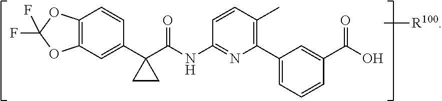

Non-limiting examples of crystal structures of ABCA4 with Protein Recognition Moieties include 7LKP and 7LKZ.

### Rhodopsin

In certain embodiments the protein stabilizing compound of the present invention includes a rhodopsin Targeting Ligand and can be used in the treatment of a rhodopsin-mediated disease such as retinitis pigmentosa, leber congenital amaurosis, or congenital night blindness.

Rhodopsin is a G-protein-coupled receptor (GCPR) expressed in rod cells of the retina and is responsible for vision in low light conditions. Within the seven transmembrane domains lies a photosensitive molecule, retinal. Upon isomerization of the alkenes within retinal, the G protein is activated causing a cGMP messenger cascade. Many retinopathies are caused by mutations in the rhodopsin gene, causing pathological ubiquitinization of rhodopsin. Ubiquitinization of rhodopsin ultimately leads to photoreceptor apoptosis and blindness.

Non-limiting examples of crystal structures of Rhodopsin 1 with Protein Recognition Moieties include 6I9K and 5AWZ. Non-limiting examples of crystal structures of Rhodopsin with Protein Recognition Moieties include 3AYM, 1L9H, 6FK6, 6FK8, 6FK7, 6FKD, 6FKC, 6FKB, 6FKA and 5TE5. Non-limiting examples of crystal structures of Rhodopsin II with Protein Recognition Moieties include 1H2S and 3AM6.

### ABCB4

In certain embodiments the protein stabilizing compound of the present invention includes an ABCB4 Targeting Ligand and can be used in the treatment of an ABCB4-mediated disease such as progressive familial intrahepatic cholestasis (PFIC), for example PFIC3.

ATP-binding cassette 4, or multidrug resistance protein 3, is a transporter protein responsible for transfer of phosphatidylcholine into the bile ducts. The phospholipid is crucial for chaperoning the bile acid into the gut, thereby protecting the duct itself. Mutations in the gene are inherited in an autosomal recessive manner and lead to progressive familial intrahepatic cholestasis-3 (PFIC-3). Patients with PFIC-3 develop bile plugs and infarcts, as well as hepatocellular injury early in childhood. If untreated the disease progresses to liver failure and death before adolescence.

### ABCB11

In certain embodiments the protein stabilizing compound of the present invention includes an ABCB11 Targeting Ligand and can be used in the treatment of an ABCB11-mediated disease such as progressive familial intrahepatic cholestasis (PFIC), for example PFIC2.

ATP-binding cassette, sub-family B member 11 (ABCB11) is a transmembrane transport protein that is responsible for bile acid homeostasis in the body. Upon binding of ATP, the triphosphate is hydrolyzed causing the transport of one molecule of cholate. Proper transport of bile acids prevents toxic buildup in hepatocytes as well as proper processing of toxins, and absorption of vitamins and fat from the diet. A deficiency in this protein causes excessive pruritis (itching), jaundice, liver cancer, leading to cirrhosis within five to ten years of life. The current treatment options are limited to invasive biliary diversion surgery or complete liver transplant.

### Dystrophin

In certain embodiments the protein stabilizing compound of the present invention includes a dystrophin Targeting Ligand and can be used in the treatment of a dystrophin-mediated disease such as muscular dystrophy for example Duchenne muscular dystrophy.

Dystrophin is a crucial structural protein responsible for the attachment of muscle cytoskeleton to the surrounding extracellular matrix. The protein is localized between the muscular cell plasma membrane (sarcolemma) and the myofiber, allowing it to attach the muscle fibers to the plasma membrane. This is the fundamental connection between tendons and the motive part of the muscular system. Due to its presence on the X chromosome, deficiencies in this gene are inherited in an X-linked recessive manner and most affected individuals are male. Dystrophin mutations cause a range of diseases known as muscular dystrophy, including Duchenne muscular dystrophy.

Antisense oligonucleotides have been examined as potential therapies, however none have been able to establish statistically significant benefit. There remains tremendous unmet medical need for patients with dystrophin mutations.

In certain embodiments, the Ubiquitinated Protein Targeting Ligand is a ligand for dystrophin selected from a small molecule, polypeptide, peptidomimetic, antibody, antibody fragment, antibody-like protein, and nucleic acid.

### P27 and P27^{Kip1}

In certain embodiments the protein stabilizing compound of the present invention includes a P27 or P27^{Kip1} Targeting Ligand and can be used in the treatment of a P27 or P27^{Kip1}-mediated disease such as a cancer for example oro-pharyngo-laryngeal cancer, esophageal cancer, gastric cancer, colon cancer, biliary tract cancer, lung cancer, melanoma, glioma, glioblastoma, breast cancer, renal cell cancer, prostate cancer, transitional cell cancer, cervix cancer, endometrial cancer, ovarian cancer, Kaposi sarcoma, soft tissue sarcoma, lymphoma, or leukemia.

P27 (encoded by the CDKN1B gene) is a cell cycle inhibitor that prevents rapid cell division. Transcription of CDKN1B is activated by FoxO, which then serves as a nuclear localization signal for P27 and decreases the levels of a P27 degrading protein COPS5. This process occurs predominantly during quiescence and early G1. To enter the cell cycle, P27 is ubiquitinated by two different proteins, SCFSKP2 kinase associate protein 1 as well as the KIP1 ubiquitylation promoting complex. These complexes polyubiquitinate P27, causing its degradation and release of inhibitory signal. Once the levels of P27 decrease, the cell begins to replicate.

Many cancers are a result of dysfunction in the synthesis, localization, or degradation of P27 and stabilizing its presence is an attractive strategy to limit replication.

Non-limiting examples of crystal structures of P27KIP1 with Protein Recognition Moieties include 3A99.

In certain embodiments the P27 or P27^{Kip1} Targeting Ligand is selected from

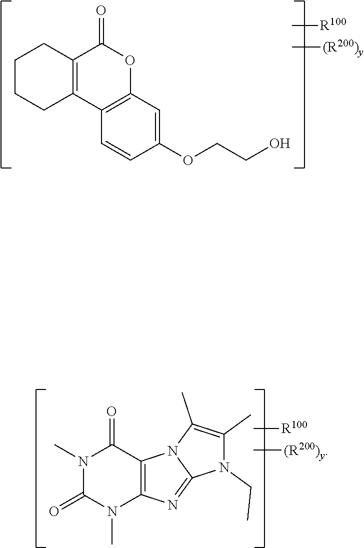

### PDCD4

In certain embodiments the protein stabilizing compound of the present invention includes a PDCD4 Targeting Ligand and can be used in the treatment of a PDCD4-mediated disease such as a cancer for example pregnancy-associated breast cancer, pancreatic cancer, lung cancer, and primary lung cancer.

Programmed cell death protein 4 (PDCD4) is a tumor suppressor protein that regulates transcription in addition to cell proliferation and tumor metastasis. PDCD4 suppresses the expression of protumor kinases JNK and MAP4K1, both proteins responsible for cell cycle initiation. PDCD4 is phosphorylated by S6 kinase (downstream of PI3K-Akt-mTOR signaling) at which point it is ubiquitinylated and then degraded. Removal of PDCD4 either through siRNA knockdown or knockout experiments shows a phenotype of aggressive cellular proliferation.

In certain embodiments the PDCD4 Targeting Ligand is a ligand described in Frankel et al. J. Biol. Chem. 2008, 283(2): 1026-1033, for example SEQ ID. 1 UAGCUUAUCAGACUGAUGUUGA.

### P53 Tumor Suppressor

In certain embodiments the protein stabilizing compound of the present invention includes a p53 Targeting Ligand and can be used in the treatment of a p53-mediated disease such as a cancer. In certain embodiments the p53 Targeting Ligand targets a p53 mutant protein. For example an amino-terminal (AT) mutation, oligomerization domain (OD) mutation, DBD mutation, or loss of function mutation.

P53 is a 43.7 kDa protein that is responsible for tumor suppression in multicellular vertebrates, and is mutated in over 50% of cancers. It plays multiple roles in preventing the development in cancers, including activation of DNA repair proteins, pausing the cell cycle to allow DNA repair to occur, and initiating apoptosis if the DNA damage is unrepairable. If p53 is mutated or otherwise inoperable, then p21 will not be produced in sufficient quantity to halt DNA replication and cell division. This allows cells with damaged DNA, a hallmark of cancer, to divide uncontrolled. In cells that are unstressed, p53 is produced but rapidly degraded through ubiquitination via Mdm2. However, when cells are stressed, the ubiquitin is cleaved and p53 is allowed to halt replication for the necessary repair processes. Given the significance of aberrant p53 regulation in cancer, it is advantageous to be able to deubiquitinate p53 to slow the growth of tumors.

In certain embodiments the p53 Targeting Ligand targets p53 with one or more mutations selected from Q136P, Y234H V272M, F270V, P278A, R213L, Y126H, T253N, T253I, R158L, Q136E, P142F, A129D, L194R, R110P, V172G, C176F, 1254N, K305R, E285D, T155P, H296D, E258G, G279V, T211A, R213P, C229Y, 1232F, E294K, P152R, R196P, M160T, N131S, N131H, K139N, L330H, Y220N, Y220C, E298Q, D148E, L64R, E224D, H168P, N263H, K320N, S227C, E286D, K292T, V203A, M237R, F212L, K132Q, Y236S, Y126S, Q136H, E221A, 1232S, Y163H, P190T, C182Y, P142L, Y163S, V218E, 1195S, V272A, and/or S106R. In certain embodiments the p53 Targeting Ligand targets Y220C p53 mutant.

Non-limiting examples of crystal structures of p53 with Protein Recognition Moieties include, 501C, 501F, 6GGA, 6GGE, 6GGC, 2VUK, 6GGN, 3ZME, 4AGN, 4AGO, 4AGM, 4AGP, 4AGQ, 5G40, and 5ABA.

c-Myc

In certain embodiments the protein stabilizing compound of the present invention includes a c-Myc Targeting Ligand and can be used in the treatment of a c-Myc-mediated disease such as a cancer. Non-limiting examples of crystal structures of c-Myc with Protein Recognition Moieties include 2L7V, 5W77, 6JJ0, 2N6C, 6UIF, 6UHZ, 6UHY, 6UJ4, 6UIK, 6UOZ.

### MSH2

In certain embodiments the protein stabilizing compound of the present invention includes a MSH2 Targeting Ligand and can be used in the treatment of a MSH2-mediated disease such as a cancer, lynch disorder, colon cancer, or endometrial cancer. DNA mismatch repair protein MSH2 is a tumor suppressor protein that forms a heterodimer with MSH6 which binds to DNA mismatches, stimulating repair. It is involved in transcription coupled repair, homologous recombination, and base excision repair. Loss of the mismatch repair system leads to microsatellite instability, an important component of colon cancer as well as others.

Non-limiting examples of crystal structures of MSH2 with Protein Recognition Moieties include 208E.

### RIPK1

In certain embodiments the protein stabilizing compound of the present invention includes a RIPK1 Targeting Ligand and can be used in the treatment of a RIPK1-mediated disease such as an inflammatory disorder, an immune disorder, an inflammatory immune disorder, cancer, or melanoma.

Receptor-interacting protein kinase 1 (RIPK1) is a serine/threonine kinase that is a crucial regulator of TNF-mediated apoptosis. RIPK1 kinase activation has been seen in samples of autoimmune and neurodegenerative conditions. RIPK1 activation begins with polyubiquitination, which then promotes the recruitment of TAK1 kinase and LUBAC complex. This complex in turn leads to necrosis and the generation of proinflammatory signaling.

Non-limiting examples of crystal structures of RIPK1 with Protein Recognition Moieties include 6NW2, 6NYH, 6AC5, 6ACI, 6C4D, 6C3E, 605Z, 6ZZ1, 5KO1, 4ITH, 4ITI, 4ITJ, 4NEU, 5HX6, 6OCQ, 6R5F, 5TX5, 6RLN, and 6HHO.

### RIPK2

In certain embodiments the protein stabilizing compound of the present invention includes a RIPK2 Targeting Ligand and can be used in the treatment of a RIPK2-mediated disease such as an inflammatory disorder, an immune disorder, an inflammatory immune disorder, cancer, or melanoma.

Receptor-interacting protein kinase 2 (RIPK2) is a serine/threoning/tyrosine kinase that is involved in immunological signaling as well as an inducer of apoptosis. Once ubiquitinated, RIPK2 recruits MAP3K7 to NEMO and this stimulates the release of NF-kappa-B, ultimately leading to activation of genes involved in cell proliferation and protection against apoptosis.

Non-limiting examples of crystal structures of RIPK1 with Protein Recognition Moieties include 6FU5, 4C8B, 5W5O, 5W5J, 6ES0, 6S1F, SYRN, 6SZJ, 6SZE, 6HMX, 6GGS, 6RNA, 6RN8, 5NG2, 5NG0, 5J7B, 5J79, 5AR8, 5AR7, 5AR5, and 5AR4.

### BAX

In certain embodiments the protein stabilizing compound of the present invention includes a BAX Targeting Ligand and can be used in the treatment of a BAX-mediated disease such as cancer, neurological disorders, neurodegenerative diseases, or inflammatory diseases.

Apoptosis regulator BAX (Bcl-2 like protein 4) is a member of the Bcl-2 family of proteins. BAX acts as an apoptotic activator through depletion of membrane potential in the mitochondria. The protein is located in the mitochondrial outer membrane. BAX deletions have been implicated in progressive neurological disorders that lead to ataxia and granule cell apoptosis. Furthermore BAX is critical in maintaining the number of B cells in both immature and mature stages.

Non-limiting examples of crystal structures of BAX with Protein Recognition Moieties include 4S0O, 3PK1, 4S0P, 4BD5, 5W63, 5W62, 4BD8, 4BD7, 5W61, 5W60, 4BD2, 3PL7. In certain embodiments the BAX stabilizing compound of the present invention is selected from:

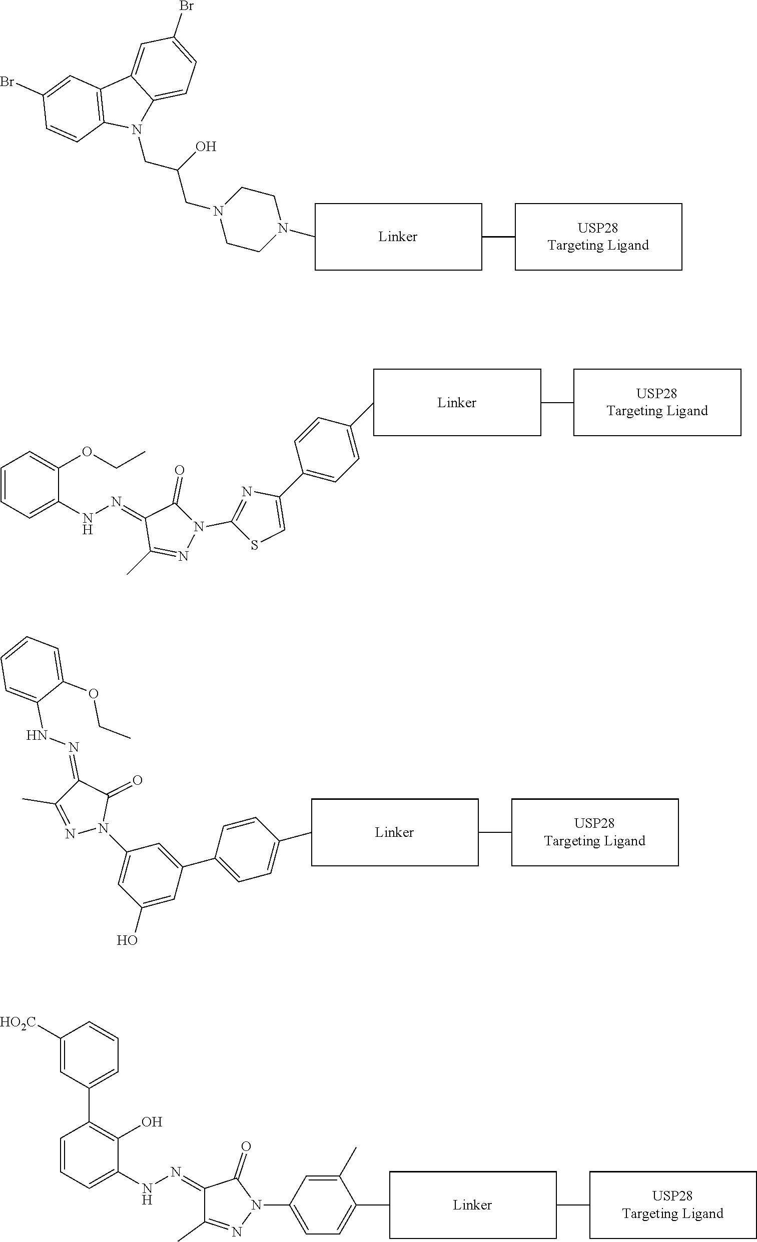 [SMILES: OC(CN1CCN(C2CCC(C3CCCCC3)CC2)CC1)Cn1c2ccc(Br)cc2c2cc(Br)ccc21 ; CCOc1ccccc1N/N=C1/C(=O)N(c2nc(-c3ccc(C4CCC(C5CCCCC5)CC4)cc3)cs2)N=C1C ; CCOc1ccccc1N/N=C1/C(=O)N(c2cc(O)cc(-c3ccc(C4CCC(C5CCCCC5)CC4)cc3)c2)N=C1C ; CC1=NN(c2ccc(C3CCC(C4CCCCC4)CC3)c(C)c2)C(=O)/C1=N/Nc1cccc(-c2cccc(C(=O)O)c2)c1O]

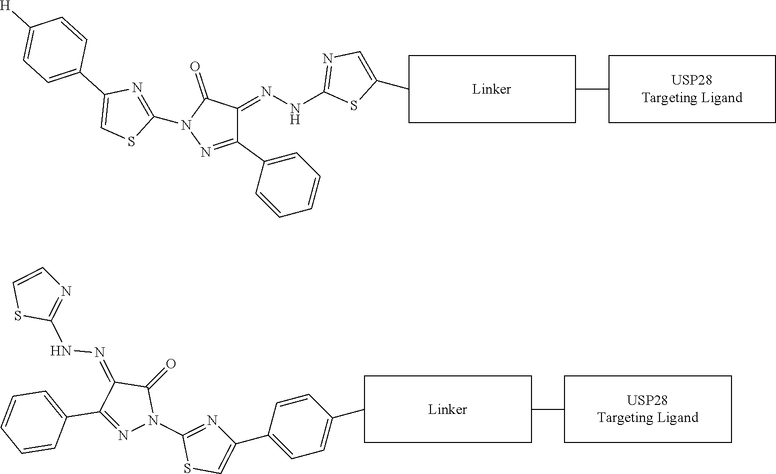 [SMILES: O=C1/C(=N/Nc2ncc(C3CCC(C4CCCCC4)CC3)s2)C(c2ccccc2)=NN1c1nc(-c2ccccc2)cs1 ; O=C1/C(=N/Nc2nccs2)C(c2ccccc2)=NN1c1nc(-c2ccc(C3CCC(C4CCCCC4)CC3)cc2)cs1]

or a pharmaceutically acceptable salt thereof.

### Alpha-Antitrypsin

In certain embodiments the protein stabilizing compound of the present invention includes an alpha antitrypsin Targeting Ligand and can be used in the treatment of an alpha antitrypsin-mediated disease such as chronic obstructive pulmonary disease, emphysema, jaundice, and liver related diseases including hepatitis and cirrhosis,

Alpha antitrypsin, encoded by the gene SERPINA1, is a serine protease inhibitor. This protein is produced by the liver and inhibits the digestive enzyme trypsin as well as neutrophil elastase. When there is insufficient alpha antitrypsin, the immune system attacks the alveolar sacs in the lungs which leads to difficulty breathing, COPD, and emphysema.

Non-limiting examples of crystal structures of alpha antitrypsin with Protein Recognition Moieties include 1D5S, 8API, 3DRM, 3DRU, 3CWL, 2QUG, 9API, 7API, 3TIP, 1HP7, 3CWM, 5IO1, 1QLP, 3NE4, IATU, 1PSI, 1QMB, 1KCT, 3DNF, 3NDD, 7AEL, 1IZ2, 1OO8, 1OPH, and 1EZX,

### PKLR

In certain embodiments the protein stabilizing compound of the present invention includes a PKLR Targeting Ligand and can be used in the treatment of a PKLR-mediated disease such as chronic hereditary nonspherocytic hemolytic anemia, jaundice, fatigue, dyspnea, Gilbert syndrome, and bone fractures.

PKLR (pyruvate kinase L/R) is a protein that catalyzes the transphosphorylation of phosphoenolpyruvate into pyruvate and ATP. This is the rate limiting step in glycolysis and leads to a lack of ATP in red blood cells. The red blood cells dehydrate and form altered shapes, which leads to hemolytic anemia.

Non-limiting examples of crystal structures of PKLR with Protein Recognition Moieties include 6NN4, 6ECH, 6NN8, 6ECK, 2VGI, 2VGG, 2VGF, 2VGB, 6NN7, 6NN5 41P7, and 4IMA.

In certain embodiments the PKLR stabilizing compound of the present invention is selected from:

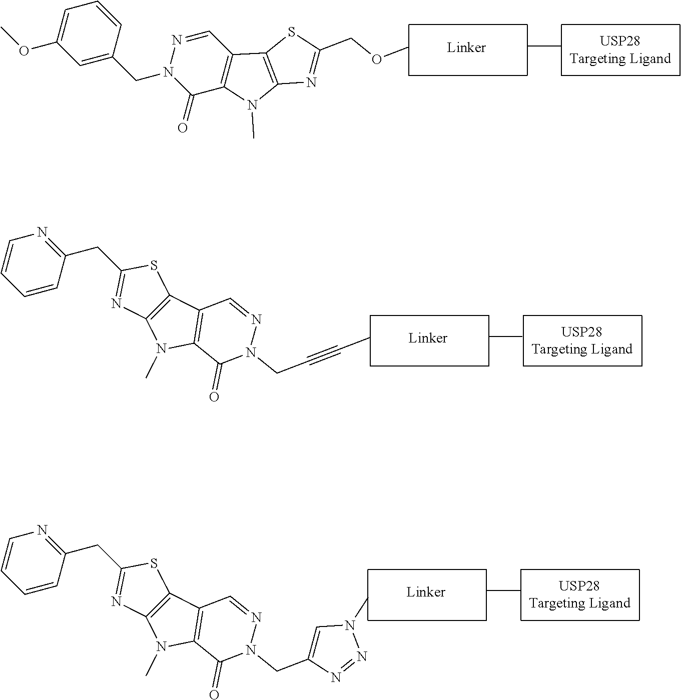

or a pharmaceutically acceptable salt thereof.

### KEAP1

In certain embodiments the protein stabilizing compound of the present invention includes a KEAP1 Targeting Ligand and can be used in the treatment of a KEAP1-mediated disease such as inflammation, chronic kidney disease, hepatocellular carcinoma and lung cancer.

KEAP1 (Kelch-like ECH-associated protein 1) regulates the activity of a BCR E3 ubiquitin ligase complex. This protein complex is responsible for responding to oxidative stress by regulating the expression of cytoprotective genes. The protein has four domains, including one domain responsible for stress signaling. This domain contains a number of cysteine residues which undergo Michael addition to reactive electrophilic species in the cell, activating KEAPl. Non-limiting examples of crystal structures of KEAP1 with Protein Recognition Moieties include 6LRZ, 7C60, 7C5E, 2Z32, 5FZN, 5FZJ, 5FNU, 5FNT, 5FNS, 5FNR, 5FNQ, 1X2J, 4CXT, 6ZEZ, 4CXJ, 7K2M, 7K2L, 7K2J, 7K2I, 6ZF8, 6ZF7, 6ZF6, 6ZF5, 6ZF4, 6ZF3, 6ZF2, 6ZF1, 6ZF0, 6ZEY, 6SP4, 6SP1, 5CGJ, 4IFN, 4IFJ, IU6D, 7K2S, 7K2R, 7K2Q, 7K2P, 7K20, 7K2N, 7K2H, 7K2G AND 6ZEX.

In certain embodiments the KEAP1 stabilizing compound of the present invention is selected from:

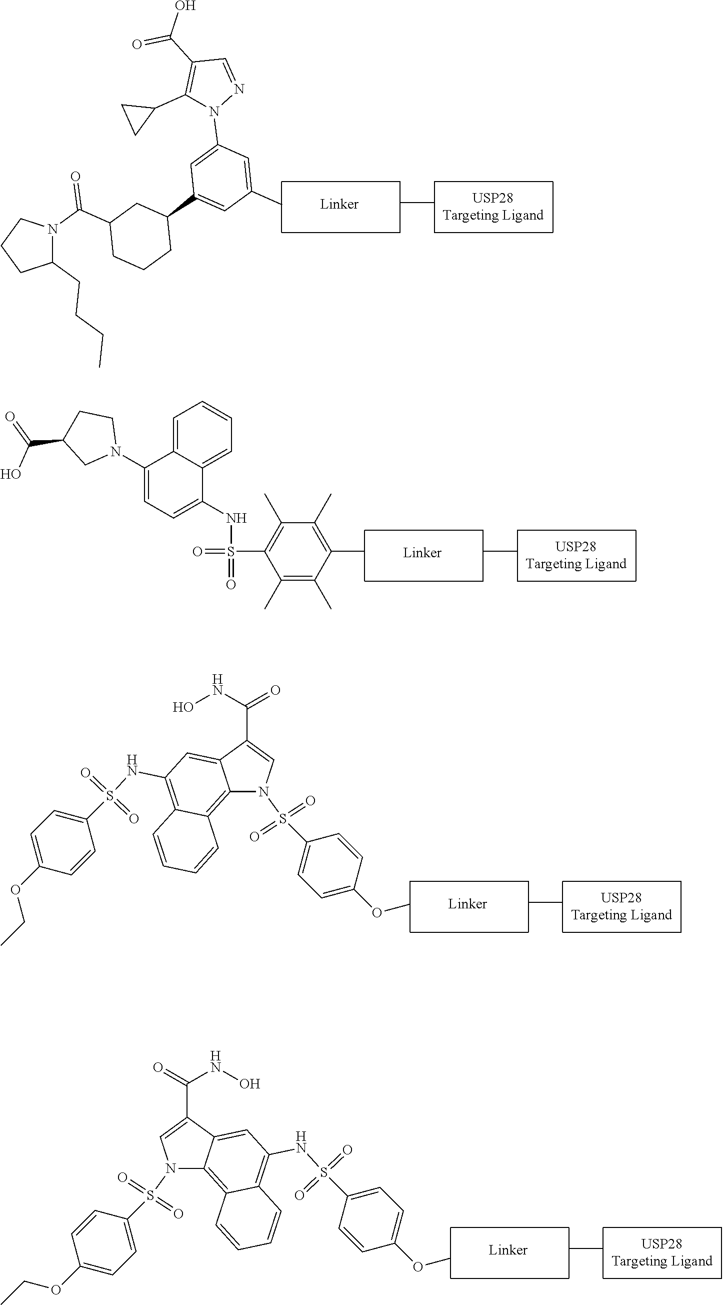

or a pharmaceutically acceptable salt thereof.

### IRAK4

In certain embodiments the protein stabilizing compound of the present invention includes a IRAK4 Targeting Ligand and can be used in the treatment of a IRAK4-mediated disease such as inflammation, infectious disease, autoimmune disease, rheumatoid arthritis and inflammatory bowel disease.

IRAK4 (interleukin-1 receptor-associated kinase 4) is a protein kinase within the toll-like receptor pathway (TLR). IRAK4 activity is required for activation of NF-kappa-B and activation of the mitogen activated protein kinase pathway that induces the cell cycle. The protein is a crucial component to an organism's response to IL-1. Without IRAK4, the animal does not adequately sense the presence of viruses or bacteria and set off the appropriate innate immune response of cytokines and chemokines. In human patients, IRAK4 deficiency presents as a defective immune system.

In certain embodiments the IRAK4 stabilizing compound of the present invention is

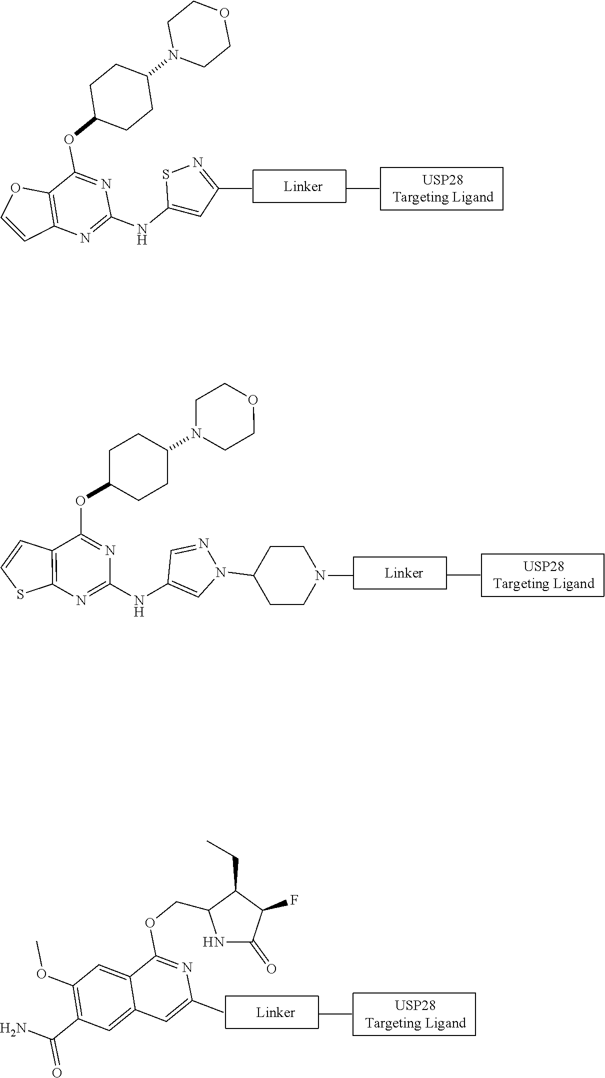

or a pharmaceutically acceptable salt thereof.

In certain embodiments the Target Ubiquitinated Protein is selected from Cystic fibrosis transmembrane conductance regulator (CFTR), Phenylalanine hydroxylase (PAH), ATP-binding cassette, sub-family A, member 4 (ABCA4), rhodopsin, ATP-Binding Cassette Sub-Family B Member 4 (ABC1B4) ATP-binding cassette, sub-family B member 11 (ABCB11), dystrophin, cyclin-dependent kinase inhibitor 1B (CDKN1B, P27, p27^{Kip1}), Programmed cell death protein 4 (PDCD4), P53, c-Myc, DNA mismatch repair protein Msh2 (MSH2), Rhodopsin, choline acetyltransferase (ChAT), NF-kappa-B essential modulator (NEMO), ubiquitin carboxy-terminal hydrolase (CYLD), aryl hydrocarbon receptor-interacting protein (AIP), Programmed cell death protein 4 (PDCD4), Receptor-interacting protein kinase 2 (RIPK2), Bcl-2-associated X protein (BAX), cyclin dependent kinase inhibitor 1A (CDKN1A, P21), alpha antitrypsin, Pyruvate kinase isozyme R/L (PKLR), Kelch like ECH associated protein 1 (KEAP1), phosphate and tensin homolog (PTEN), interleukin-1 receptor-associated kinase 4 (IRAK4), thymidine kinase 2 (TK2), potassium voltage-gated channel subfamily Q (KCNQ1), stimulator of interferon genes (STING1) and Receptor-interacting protein kinase 1 (RIPK1)

In certain embodiments the Ubiquitinated Protein Targeting Ligand binds a protein that is selected from Cystic fibrosis transmembrane conductance regulator (CFTR), Phenylalanine hydroxylase (PAH), ATP-binding cassette, sub-family A, member 4 (ABCA4), rhodopsin, ATP-Binding Cassette Sub-Farnily B Meinber 4 (ABCB4), ATP-binding cassette, sub-family B member 11 (ABCB11), dystrophin, cyclin-dependent kinase inhibitor 1B (CDKN1B, P27, p27^{Kip1}) Programmed cell death protein 4 (PDCD4), P53, c-Myc, DNA mismatch repair protein Msh2 (MSH2), Rhodopsin, choline acetyltransferase (ChAT), NF-kappa-B essential modulator (NEMO), ubiquitin carboxy-terminal hydrolase (CYLD), aryl hydrocarbon receptor-interacting protein (AIP), Programmed cell death protein 4 (PDCD4), Receptor-interacting protein kinase 2 (RIPK2), Bcl-2-associated X protein (BAX), cyclin dependent kinase inhibitor 1A (CDKN1A, P21), alpha antitrypsin, Pyruvate kinase isozyme R/L (PKLR), Kelch like ECH associated protein 1 (KEAP1), phosphate and tensin homolog (PTEN), interleukin-1 receptor-associated kinase 4 (IRAK4), thymidine kinase 2 (TK2), potassium voltage-gated channel subfamily Q (KCNQ1), stimulator of interferon genes (STING1) and Receptor-interacting protein kinase 1 (RIPK1)

### Methods of Treatment

A protein stabilizing compound described herein can be used to treat a disorder mediated by a Target Ubiquitinated Protein. For example, when restoring the function of the Target Ubiquitinated Protein ameliorates a cancer than the protein stabilizing compound can be used in the treatment of that cancer.

In certain embodiments, the Target Ubiquitinated Protein is the wild type protein. In certain embodiments, the Target Ubiquitinated Protein is a mutant protein. In certain embodiments, the Target Ubiquitinated Protein is in a prokaryotic or eukaryotic cell. In certain embodiments, the Target Ubiquitinated Protein is in a eukaryotic cell that is within a multicellular organism. In certain embodiments, the Target Ubiquitinated Protein is in an animal, including but not limited to humans.

Exemplary cancers which may be treated by a disclosed protein stabilizing compound either alone or in combination with at least one additional anti-cancer agent include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using the a disclosed protein stabilizing compound according to the present invention include, for example, acute granulocytic leukemia, acute lymphocytic leukemia (ALL), acute myelogenous leukemia (AML), adenocarcinoma, adenosarcoma, adrenal cancer, adrenocortical carcinoma, anal cancer, anaplastic astrocytoma, angiosarcoma, appendix cancer, astrocytoma, Basal cell carcinoma, B-Cell lymphoma, bile duct cancer, bladder cancer, bone cancer, bone marrow cancer, bowel cancer, brain cancer, brain stem glioma, breast cancer, triple (estrogen, progesterone and HER-2) negative breast cancer, double negative breast cancer (two of estrogen, progesterone and HER-2 are negative), single negative (one of estrogen, progesterone and HER-2 is negative), estrogen-receptor positive, HER2-negative breast cancer, estrogen receptor-negative breast cancer, estrogen receptor positive breast cancer, metastatic breast cancer, luminal A breast cancer, luminal B breast cancer, Her2-negative breast cancer, HER2-positive or negative breast cancer, progesterone receptor-negative breast cancer, progesterone receptor-positive breast cancer, recurrent breast cancer, carcinoid tumors, cervical cancer, cholangiocarcinoma, chondrosarcoma, chronic lymphocytic leukemia (CLL), chronic myelogenous leukemia (CML), colon cancer, colorectal cancer, craniopharyngioma, cutaneous lymphoma, cutaneous melanoma, diffuse astrocytoma, ductal carcinoma in situ (DCIS), endometrial cancer, ependymoma, epithelioid sarcoma, esophageal cancer, ewing sarcoma, extrahepatic bile duct cancer, eye cancer, fallopian tube cancer, fibrosarcoma, gallbladder cancer, gastric cancer, gastrointestinal cancer, gastrointestinal carcinoid cancer, gastrointestinal stromal tumors (GIST), germ cell tumor glioblastoma multiforme (GBM), glioblastoma, recurrent glioblastoma, glioma, hairy cell leukemia, head and neck cancer, hemangioendothelioma, Hodgkin lymphoma, hypopharyngeal cancer, infiltrating ductal carcinoma (IDC), infiltrating lobular carcinoma (ILC), inflammatory breast cancer (IBC), intestinal Cancer, intrahepatic bile duct cancer, invasive/infiltrating breast cancer, Islet cell cancer, jaw cancer, Kaposi sarcoma, kidney cancer, laryngeal cancer, leiomyosarcoma, leptomeningeal metastases, leukemia, lip cancer, liposarcoma, liver cancer, lobular carcinoma in situ, low-grade astrocytoma, lung cancer, lymph node cancer, lymphoma, male breast cancer, medullary carcinoma, medulloblastoma, melanoma, meningioma, Merkel cell carcinoma, mesenchymal chondrosarcoma, mesenchymous, mesothelioma metastatic breast cancer, metastatic melanoma metastatic squamous neck cancer, mixed gliomas, monodermal teratoma, mouth cancer mucinous carcinoma, mucosal melanoma, multiple myeloma, Mycosis Fungoides, myelodysplastic syndrome, nasal cavity cancer, nasopharyngeal cancer, neck cancer, neuroblastoma, neuroendocrine tumors (NETs), non-Hodgkin's lymphoma, non-small cell lung cancer (NSCLC), oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer ovarian germ cell tumor, ovarian primary peritoneal carcinoma, ovarian sex cord stromal tumor, Paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pineoblastoma, pituitary gland cancer, primary central nervous system (CNS) lymphoma, prostate cancer, rectal cancer, renal cell carcinoma, renal pelvis cancer, rhabdomyosarcoma, salivary gland cancer, soft tissue sarcoma, bone sarcoma, sarcoma, sinus cancer, skin cancer, small cell lung cancer (SCLC), small intestine cancer, spinal cancer, spinal column cancer, spinal cord cancer, squamous cell carcinoma, stomach cancer, synovial sarcoma, T-cell lymphoma, testicular cancer, throat cancer, thymoma/thymic carcinoma, thyroid cancer, tongue cancer, tonsil cancer, transitional cell cancer, tubal cancer, tubular carcinoma, undiagnosed cancer, ureteral cancer, urethral cancer, uterine adenocarcinoma, uterine cancer, uterine sarcoma, vaginal cancer, vulvar cancer, T-cell lineage acute lymphoblastic leukemia (T-ALL), T-cell lineage lymphoblastic lymphoma (T-LL), peripheral T-cell lymphoma, Adult T-cell leukemia, Pre-B ALL, Pre-B lymphomas, large B-cell lymphoma, Burkitts lymphoma, B-cell ALL, Philadelphia chromosome positive ALL, Philadelphia chromosome positive CML, juvenile myelomonocytic leukemia (JMML), acute promyelocytic leukemia (a subtype of AML), large granular lymphocytic leukemia, Adult T-cell chronic leukemia, diffuse large B cell lymphoma, follicular lymphoma; Mucosa-Associated Lymphatic Tissue lymphoma (MALT), small cell lymphocytic lymphoma, mediastinal large B cell lymphoma, nodal marginal zone B cell lymphoma (NMZL); splenic marginal zone lymphoma (SMZL); intravascular large B-cell lymphoma; primary effusion lymphoma; or lymphomatoid granulomatosis; B-cell prolymphocytic leukemia; splenic lymphoma/leukemia, unclassifiable, splenic diffuse red pulp small B-cell lymphoma; lymphoplasmacytic lymphoma; heavy chain diseases, for example, Alpha heavy chain disease, Gamma heavy chain disease, Mu heavy chain disease, plasma cell myeloma, solitary plasmacytoma of bone; extraosseous plasmacytoma; primary cutaneous follicle center lymphoma, T cell/histocyte rich large B-cell lymphoma, DLBCL associated with chronic inflammation; Epstein-Barr virus (EBV)+DLBCL of the elderly; primary mediastinal (thymic) large B-cell lymphoma, primary cutaneous DLBCL, leg type, ALK+large B-cell lymphoma, plasmablastic lymphoma; large B-cell lymphoma arising in HHV8-associated multicentric, Castleman disease; B-cell lymphoma, unclassifiable, with features intermediate between diffuse large B-cell lymphoma, or B-cell lymphoma, unclassifiable, with features intermediate between diffuse large B-cell lymphoma and classical Hodgkin lymphoma (Yu et al., “DNA damage induces cdk2 protein levels and histone H2B phosphorylation in SH-SY5Y neuroblastoma cells”, J Alzheimer's Dis., 2005 September; 8(1):7-21).

Additional, non-limiting examples of cancers that can be treated according to the present invention include, but are not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL)—also known as acute lymphoblastic leukemia or acute lymphoid leukemia (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In certain embodiments, the cancer is a hematopoietic cancer. In certain embodiments, the hematopoietic cancer is a lymphoma. In certain embodiments, the hematopoietic cancer is a leukemia. In certain embodiments, the leukemia is acute myelocytic leukemia (AML).

In certain embodiments, the proliferative disorder is a myeloproliferative neoplasm. In certain embodiments, the myeloproliferative neoplasm (MPN) is primary myelofibrosis (PMF).

In certain embodiments, the cancer is a solid tumor. A solid tumor, as used herein, refers to an abnormal mass of tissue that usually does not contain cysts or liquid areas. Different types of solid tumors are named for the type of cells that form them. Examples of classes of solid tumors include, but are not limited to, sarcomas, carcinomas, and lymphomas, as described above herein. Additional examples of solid tumors include, but are not limited to, squamous cell carcinoma, colon cancer, breast cancer, prostate cancer, lung cancer, liver cancer, pancreatic cancer, and melanoma.

In certain embodiments the disorder is a renal disease.

Non-limiting examples of renal disease include hypomagnesaemia type 2, hypomagnesaemia type 3, hypomagnesaemia type 5, uromodulin-associated kidney disease, gitelman syndrome, distal renal tubular acidosis, Liddle syndrome, nephrogenic diabetes insipidus, cystic fibrosis, Fabry disease, Alport syndrome, hereditary angiopathy with nephropathy aneurysms and muscle cramps (HANAC), focal segmental glomerulosclerosis 1, focal segmental glomerulosclerosis 2, focal segmental glomerulosclerosis 5, focal segmental glomerulosclerosis 6, nephrotic syndrome type 1, nephrotic syndrome type 2, Pierson syndrome, cystinosis, cystinuria type A, Dent's disease 1, Dent's disease 2, hypophosphataemic rickets with hypercalciuria, hypophosphataemic rickets, Lowe syndrome, proximal renal tubular acidosis, renal glucosuria, Bartter syndrome antenatal type 1, Bartter syndrome antenatal type 2, and Bartter syndrome type 4.

In certain embodiments the disorder is cystic fibrosis.

In certain embodiments the disorder is phenylketonuria (PKU), non-PKU hyperphenylalaninemia (HPA), or variant PKU.

In certain embodiments the disorder is Stargardt disease or retinal degeneration.

In certain embodiments the disorder is retinitis pigmentosa, leber congenital amaurosis, or congenital night blindness.

In certain embodiments the disorder is progressive familial intrahepatic cholestasis (PFIC).

In certain embodiments the disorder is muscular dystrophy for example Duchenne muscular dystrophy.

In certain embodiments the disorder is oro-pharyngo-laryngeal cancer, esophageal cancer, gastric cancer, colon cancer, biliary tract cancer, lung cancer, melanoma, glioma, glioblastoma, breast cancer, renal cell cancer, prostate cancer, transitional cell cancer, cervix cancer, endometrial cancer, ovarian cancer, Kaposi sarcoma, soft tissue sarcoma, lymphoma, or leukemia.

In certain embodiments the disorder is pregnancy-associated breast cancer, pancreatic cancer, lung cancer, and primary lung cancer.

In certain embodiments the disorder is inflammatory disorder, an immune disorder, an inflammatory immune disorder, cancer, or melanoma.

### Linker

The USP28 Targeting Ligand and Ubiquitinated Protein Targeting Ligand are linked by a Linker group.

In certain embodiments the Linker-USP28 Targeting Ligand or Linker-Ubiquitinated Protein Targeting Ligand replaces an atom, for example a halogen, alkyl, hydroxy, alkoxy, cyano, or nitro group. For example wherein Linker is

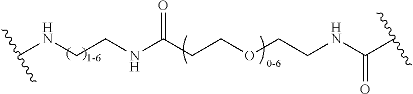

### and the USP28 Targeting Ligand is

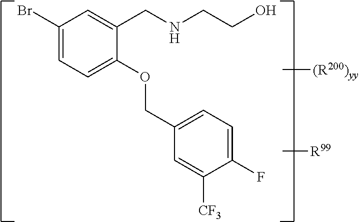

the Linker group can replace the bromine group to form the following compound:

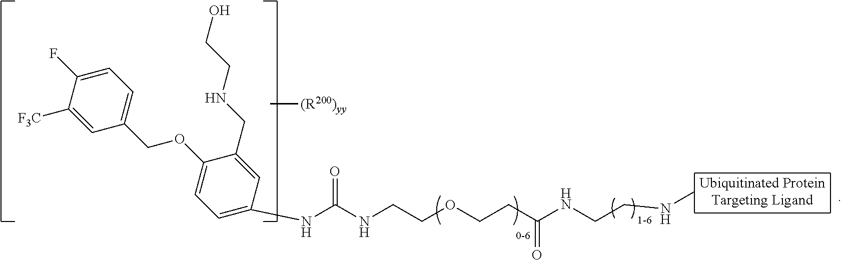

In certain embodiments the Linker-USP28 Targeting Ligand or Linker-Ubiquitinated Protein Targeting Ligand replaces a halogen.

In certain embodiments the Linker-USP28 Targeting Ligand or Linker-Ubiquitinated Protein Targeting Ligand replaces an iodine.

In certain embodiments the Linker-USP28 Targeting Ligand or Linker-Ubiquitinated Protein Targeting Ligand replaces a bromine.

In certain embodiments the Linker-USP28 Targeting Ligand or Linker-Ubiquitinated Protein Targeting Ligand replaces a chlorine.

In certain embodiments the Linker-USP28 Targeting Ligand or Linker-Ubiquitinated Protein Targeting Ligand replaces a fluorine.

In certain embodiments the Linker-USP28 Targeting Ligand or Linker-Ubiquitinated Protein Targeting Ligand replaces an alkyl.

In certain embodiments the Linker-USP28 Targeting Ligand or Linker-Ubiquitinated Protein Targeting Ligand replaces a methyl

In certain embodiments the Linker-USP28 Targeting Ligand or Linker-Ubiquitinated Protein Targeting Ligand replaces a ethyl

In certain embodiments the Linker-USP28 Targeting Ligand or Linker-Ubiquitinated Protein Targeting Ligand replaces an alkoxy.

In certain embodiments the Linker-USP28 Targeting Ligand or Linker-Ubiquitinated Protein Targeting Ligand replaces a cyano.

In certain embodiments the Linker-USP28 Targeting Ligand or Linker-Ubiquitinated Protein Targeting Ligand replaces a nitro.

Non-limiting examples of Linkers that can be used in a protein stabilizing compound of the present invention are exemplified by the compounds drawn herein and the following embodiments.
1. In certain embodiments Linker is:

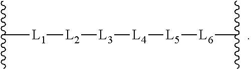

2. The Linker of embodiment 1, wherein L₁ is bond.
3. The Linker of embodiment 1, wherein L₁ is alkyl optionally substituted with 1, 2, 3, or 4 substituents independently selected from R⁴⁴.
4. The Linker of embodiment 1, wherein L₁ is alkene optionally substituted with 1, 2, 3, or 4 substituents independently selected from R⁴⁴.
5. The Linker of embodiment 1, wherein L₁ is alkyne optionally substituted with 1, 2, 3, or 4 substituents independently selected from R⁴⁴.
6. The Linker of embodiment 1, wherein L₁ is haloalkyl optionally substituted with 1, 2, 3, or 4 substituents independently selected from R⁴⁴.
7. The Linker of embodiment 1, wherein L₁ is aryl optionally substituted with 1, 2, 3, or 4 substituents independently selected from R⁴⁴.
8. The Linker of embodiment 1, wherein L₁ is heterocycle optionally substituted with 1, 2, 3, or 4 substituents independently selected from R⁴⁴.
9. The Linker of embodiment 1, wherein L₁ is heteroaryl optionally substituted with 1, 2, 3, or 4 substituents independently selected from R⁴⁴.
10. The Linker of embodiment 1, wherein L₁ is bicycle optionally substituted with 1, 2, 3, or 4 substituents independently selected from R⁴⁴.
11. The Linker of embodiment 1, wherein L₁ is —C(O)—.
12. The Linker of embodiment 1, wherein L₁ is —C(O)O—.
13. The Linker of embodiment 1, wherein L₁ is —OC(O)—.
14. The Linker of embodiment 1, wherein L₁ is —SO₂—.
15. The Linker of embodiment 1, wherein L₁ is —S(O)—.
16. The Linker of embodiment 1, wherein L₁ is —C(S)—.
17. The Linker of embodiment 1, wherein L₁ is —C(O)NR¹¹—.
18. The Linker of embodiment 1, wherein L₁ is —NR¹¹C(O)—.
19. The Linker of embodiment 1, wherein L₁ is —O—.
20. The Linker of embodiment 1, wherein L₁ is —S—.
21. The Linker of embodiment 1, wherein L₁ is —NR¹¹—.
22. The Linker of embodiment 1, wherein L₁ is —P(O)(OR¹¹)O—.
23. The Linker of embodiment 1, wherein L₁ is —P(O)(OR¹¹)—.
24. The Linker of embodiment 1, wherein L₁ is polyethylene glycol.
25. The Linker of embodiment 1, wherein L₁ is lactic acid.
26. The Linker of embodiment 1, wherein L₁ is glycolic acid.
27. The Linker of any one of embodiments 1-26, wherein L₂ is bond.
28. The Linker of any one of embodiments 1-26, wherein L₂ is alkyl optionally substituted with 1, 2, 3, or 4 substituents independently selected from R⁴⁴.
29. The Linker of any one of embodiments 1-26, wherein L₂ is alkene optionally substituted with 1, 2, 3, or 4 substituents independently selected from R⁴⁴.
30. The Linker of any one of embodiments 1-26, wherein L₂ is alkyne optionally substituted with 1, 2, 3, or 4 substituents independently selected from R⁴⁴.
31. The Linker of any one of embodiments 1-26, wherein L₂ is haloalkyl optionally substituted with 1, 2, 3, or 4 substituents independently selected from R⁴⁴.
32. The Linker of any one of embodiments 1-26, wherein L₂ is aryl optionally substituted with 1, 2, 3, or 4 substituents independently selected from R⁴⁴.
33. The Linker of any one of embodiments 1-26, wherein L₂ is heterocycle optionally substituted with 1, 2, 3, or 4 substituents independently selected from R⁴⁴.
34. The Linker of any one of embodiments 1-26, wherein L₂ is heteroaryl optionally substituted with 1, 2, 3, or 4 substituents independently selected from R⁴⁴.
35. The Linker of any one of embodiments 1-26, wherein L₂ is bicycle optionally substituted with 1, 2, 3, or 4 substituents independently selected from R⁴⁴.
36. The Linker of any one of embodiments 1-35, wherein L₃ is bond.
37. The Linker of any one of embodiments 1-35, wherein L₃ is alkyl optionally substituted with 1, 2, 3, or 4 substituents independently selected from R⁴⁴.
38. The Linker of any one of embodiments 1-35, wherein L₃ is alkene optionally substituted with 1, 2, 3, or 4 substituents independently selected from R⁴⁴.
39. The Linker of any one of embodiments 1-35, wherein L₃ is alkyne optionally substituted with 1, 2, 3, or 4 substituents independently selected from R⁴⁴.
40. The Linker of any one of embodiments 1-35, wherein L₃ is haloalkyl optionally substituted with 1, 2, 3, or 4 substituents independently selected from R⁴⁴.
41. The Linker of any one of embodiments 1-35, wherein L₃ is aryl optionally substituted with 1, 2, 3, or 4 substituents independently selected from R⁴⁴.
42. The Linker of any one of embodiments 1-35, wherein L₃ is heterocycle optionally substituted with 1, 2, 3, or 4 substituents independently selected from R⁴⁴.
43. The Linker of any one of embodiments 1-35, wherein L₃ is heteroaryl optionally substituted with 1, 2, 3, or 4 substituents independently selected from R⁴⁴.
44. The Linker of any one of embodiments 1-35, wherein L₃ is bicycle optionally substituted with 1, 2, 3, or 4 substituents independently selected from R⁴⁴.
45. The Linker of any one of embodiments 1-35, wherein L₃ is —C(O)—.
46. The Linker of any one of embodiments 1-35, wherein L₃ is —C(O)O—.
47. The Linker of any one of embodiments 1-35, wherein L₃ is —OC(O)—.
48. The Linker of any one of embodiments 1-35, wherein L₃ is —SO₂—.
49. The Linker of any one of embodiments 1-35, wherein L₃ is —S(O)—.
50. The Linker of any one of embodiments 1-35, wherein L₃ is —C(S)—.
51. The Linker of any one of embodiments 1-35, wherein L₃ is —C(O)NR¹¹—.
52. The Linker of any one of embodiments 1-35, wherein L₃ is —NR¹¹C(O)—.
53. The Linker of any one of embodiments 1-35, wherein L₃ is —O—.
54. The Linker of any one of embodiments 1-35, wherein L₃ is —S—.
55. The Linker of any one of embodiments 1-35, wherein L₃ is —NR¹¹—.
56. The Linker of any one of embodiments 1-35, wherein L₃ is —P(O)(OR¹¹)O—.
57. The Linker of any one of embodiments 1-35, wherein L₃ is —P(O)(OR¹¹)—.
58. The Linker of any one of embodiments 1-35, wherein L₃ is polyethylene glycol.
59. The Linker of any one of embodiments 1-35, wherein L₃ is lactic acid.
60. The Linker of any one of embodiments 1-35, wherein L₃ is glycolic acid.
61. The Linker of any one of embodiments 1-60, wherein L₄ is bond.
62. The Linker of any one of embodiments 1-60, wherein L₄ is alkyl optionally substituted with 1, 2, 3, or 4 substituents independently selected from R⁴⁴.
63. The Linker of any one of embodiments 1-60, wherein L₄ is alkene optionally substituted with 1, 2, 3, or 4 substituents independently selected from R⁴⁴.
64. The Linker of any one of embodiments 1-60, wherein L₄ is alkyne optionally substituted with 1, 2, 3, or 4 substituents independently selected from R⁴⁴.
65. The Linker of any one of embodiments 1-60, wherein L₄ is haloalkyl optionally substituted with 1, 2, 3, or 4 substituents independently selected from R⁴⁴.
66. The Linker of any one of embodiments 1-60, wherein L₄ is aryl optionally substituted with 1, 2, 3, or 4 substituents independently selected from R⁴⁴.
67. The Linker of any one of embodiments 1-60, wherein L₄ is heterocycle optionally substituted with 1, 2, 3, or 4 substituents independently selected from R⁴⁴.
68. The Linker of any one of embodiments 1-60, wherein L₄ is heteroaryl optionally substituted with 1, 2, 3, or 4 substituents independently selected from R⁴⁴.
69. The Linker of any one of embodiments 1-60, wherein L₄ is bicycle optionally substituted with 1, 2, 3, or 4 substituents independently selected from R⁴⁴.
70. The Linker of any one of embodiments 1-69, wherein L₅ is bond.
71. The Linker of any one of embodiments 1-69, wherein L₅ is alkyl optionally substituted with 1, 2, 3, or 4 substituents independently selected from R⁴⁴.
72. The Linker of any one of embodiments 1-69, wherein L₅ is alkene optionally substituted with 1, 2, 3, or 4 substituents independently selected from R⁴⁴.
73. The Linker of any one of embodiments 1-69, wherein L₅ is alkyne optionally substituted with 1, 2, 3, or 4 substituents independently selected from R⁴⁴.
74. The Linker of any one of embodiments 1-69, wherein L₅ is haloalkyl optionally substituted with 1, 2, 3, or 4 substituents independently selected from R⁴⁴.
75. The Linker of any one of embodiments 1-69, wherein L₅ is aryl optionally substituted with 1, 2, 3, or 4 substituents independently selected from R⁴⁴.
76. The Linker of any one of embodiments 1-69, wherein L₅ is heterocycle optionally substituted with 1, 2, 3, or 4 substituents independently selected from R⁴⁴.
77. The Linker of any one of embodiments 1-69, wherein L₅ is heteroaryl optionally substituted with 1, 2, 3, or 4 substituents independently selected from R⁴⁴.
78. The Linker of any one of embodiments 1-69, wherein L₅ is bicycle optionally substituted with 1, 2, 3, or 4 substituents independently selected from R⁴⁴.
79. The Linker of any one of embodiments 1-78, wherein L₆ is bond.
80. The Linker of any one of embodiments 1-78, wherein L₆ is alkyl optionally substituted with 1, 2, 3, or 4 substituents independently selected from R⁴⁴.
81. The Linker of any one of embodiments 1-78, wherein L₆ is alkene optionally substituted with 1, 2, 3, or 4 substituents independently selected from R⁴⁴.
82. The Linker of any one of embodiments 1-78, wherein L₆ is alkyne optionally substituted with 1, 2, 3, or 4 substituents independently selected from R⁴⁴.
83. The Linker of any one of embodiments 1-78, wherein L₆ is haloalkyl optionally substituted with 1, 2, 3, or 4 substituents independently selected from R⁴⁴.
84. The Linker of any one of embodiments 1-78, wherein L₆ is aryl optionally substituted with 1, 2, 3, or 4 substituents independently selected from R⁴⁴.
85. The Linker of any one of embodiments 1-78, wherein L₆ is heterocycle optionally substituted with 1, 2, 3, or 4 substituents independently selected from R⁴⁴.
86. The Linker of any one of embodiments 1-78, wherein L₆ is heteroaryl optionally substituted with 1, 2, 3, or 4 substituents independently selected from R⁴⁴.
87. The Linker of any one of embodiments 1-78, wherein L₆ is bicycle optionally substituted with 1, 2, 3, or 4 substituents independently selected from R⁴⁴.
88. The Linker of any one of embodiments 1-87, wherein L₁ is bound to USP28 Targeting Ligand.
89. The Linker of any one of embodiments 1-87, wherein L₁ is bound to Ubiquitinated Protein Targeting Ligand.
90. The Linker of any one of embodiments 1-89, wherein R⁴⁴ is independently selected at each instance from alkyl, halogen, and haloalkyl.
91. The Linker of any one of embodiments 1-89, wherein R⁴⁴ is alkyl optionally substituted as allowed by valence with 1, 2, 3, or 4 substituents selected from R⁴⁵.
92. The Linker of any one of embodiments 1-89, wherein R⁴⁴ is aryl optionally substituted as allowed by valence with 1, 2, 3, or 4 substituents selected from R⁴⁵.
93. The Linker of any one of embodiments 1-89, wherein R⁴⁴ is heterocycle optionally substituted as allowed by valence with 1, 2, 3, or 4 substituents selected from R⁴⁵.
94. The Linker of any one of embodiments 1-89, wherein R⁴⁴ is heteroaryl optionally substituted as allowed by valence with 1, 2, 3, or 4 substituents selected from R⁴⁵.
95. The Linker of any one of embodiments 1-89, wherein R⁴⁴ is amino optionally substituted as allowed by valence with 1, 2, 3, or 4 substituents selected from R⁴⁵.
96. The Linker of any one of embodiments 1-89, wherein R⁴⁴ is hydroxyl optionally substituted as allowed by valence with 1, 2, 3, or 4 substituents selected from R⁴⁵.
97. The Linker of any one of embodiments 1-89, wherein R⁴⁴ is alkoxy optionally substituted as allowed by valence with 1, 2, 3, or 4 substituents selected from R⁴⁵.
98. The Linker of any one of embodiments 1-89, wherein R⁴⁴ is cyano.
99. The Linker of any one of embodiments 1-89, wherein R⁴⁴ is nitro.
100. The Linker of any one of embodiments 1-89, wherein R⁴⁴ is —OC(O)R⁴⁰.
101. The Linker of any one of embodiments 1-89, wherein R⁴⁴ is —NR¹¹C(O)R⁴⁰.
102. The Linker of any one of embodiments 1-89, wherein R⁴⁴ is —C(O)R⁴⁰.
103. The Linker of any one of embodiments 1-89, wherein R⁴⁴ is —OP(O)(R⁴⁰)₂.
104. The Linker of any one of embodiments 1-89, wherein R⁴⁴ is —P(O)(R⁴⁰)₂.
105. The Linker of any one of embodiments 1-89, wherein R⁴⁴ is —NR¹¹P(O)(R⁴⁰)₂.
106. The Linker of any one of embodiments 1-89, wherein R⁴⁴ is —SR¹¹.
107. The Linker of any one of embodiments 1-89, wherein R⁴⁴ is —OR¹¹.
108. The Linker of any one of embodiments 1-89, wherein R⁴⁴ is —S(O)R⁴⁰.
109. The Linker of any one of embodiments 1-89, wherein R⁴⁴ is —S(O)₂R⁴⁰.
110. The Linker of any one of embodiments 1-89, wherein R⁴⁴ is —N(alkyl)C(O)R⁴⁰.
111. The Linker of any one of embodiments 90-97, wherein R⁴⁵ is independently selected from halogen, alkyl, and haloalkyl.
112. The Linker of any one of embodiments 90-97, wherein R⁴⁵ is independently selected from amino, hydroxyl, alkoxy, —NHalkyl, —N(alkyl)₂, —OC(O)alkyl, —NHC(O)alkyl, and —N(alkyl)C(O)alkyl.

In certain embodiments, Linker is selected from:

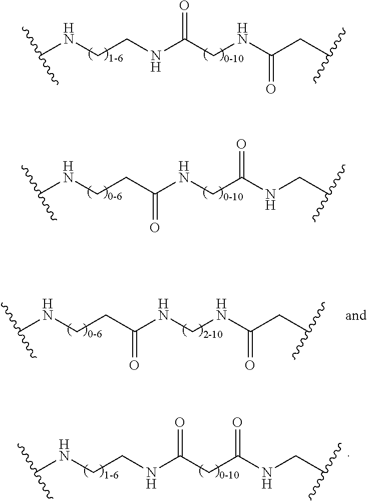

In certain embodiments, Linker is selected from:

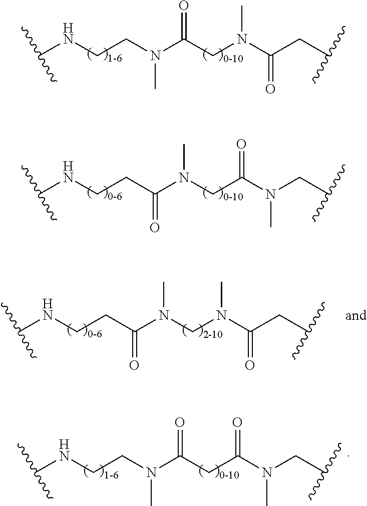

In certain embodiments, Linker is selected from:

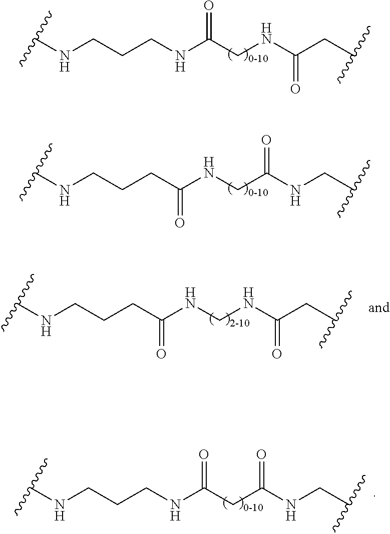

In certain embodiments, Linker is selected from:

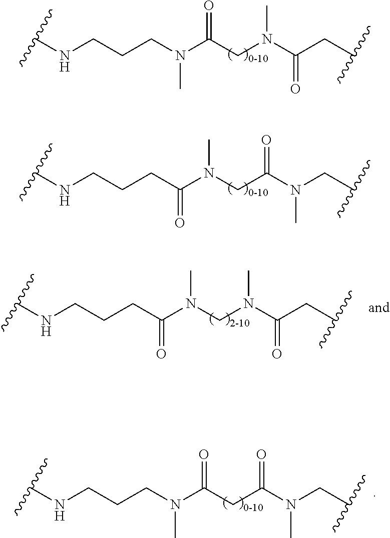

In certain embodiments, Linker is selected from:

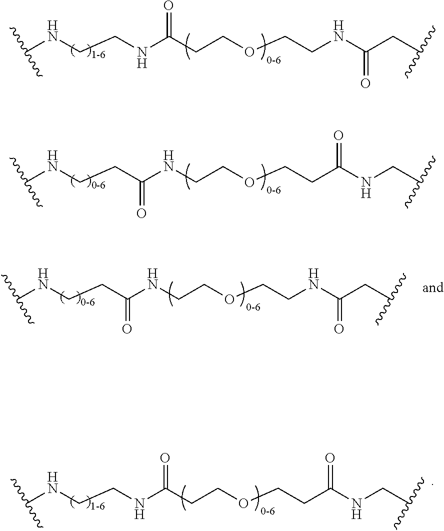

In certain embodiments, Linker is selected from:

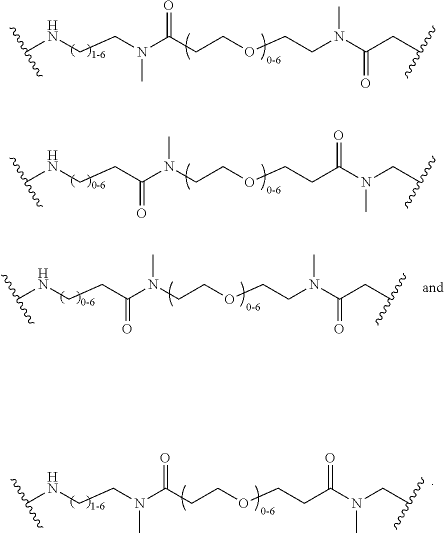

In certain embodiments, Linker is selected from:

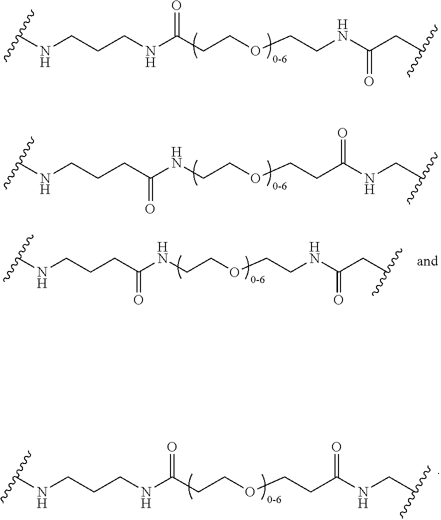

In certain embodiments, Linker is selected from:

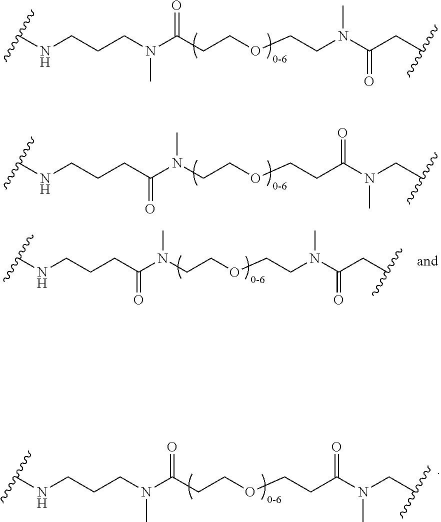

In certain embodiments, Linker is selected from:

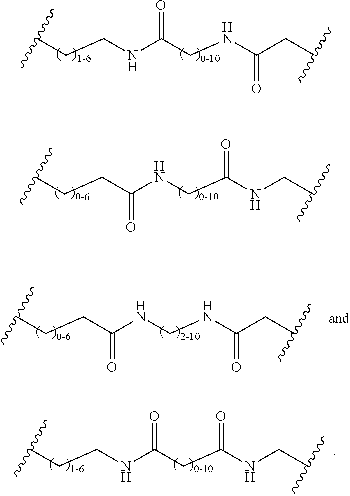

In certain embodiments, Linker is selected from:

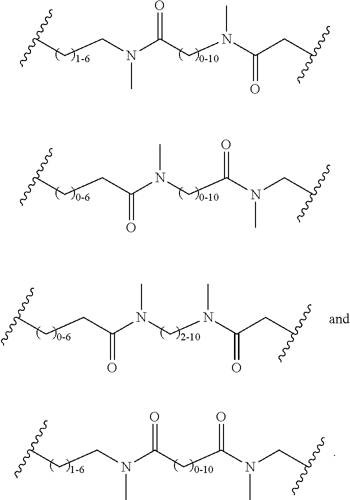

In certain embodiments, Linker is selected from:

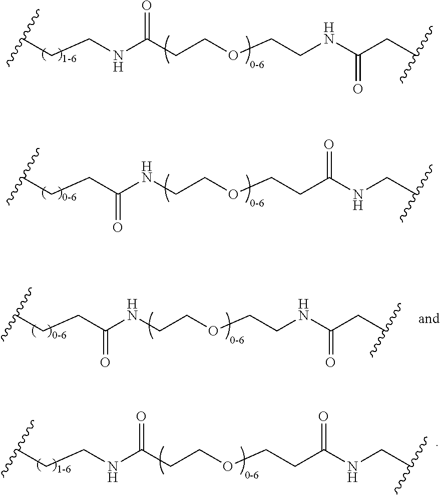

In certain embodiments, Linker is selected from:

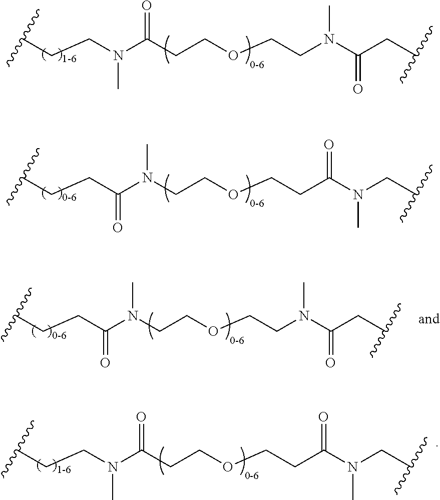

In certain embodiments, Linker is selected from:

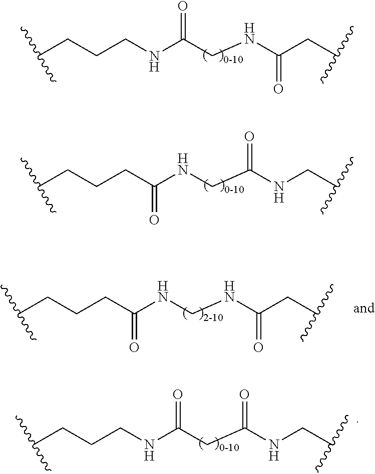

In certain embodiments, Linker is selected from:

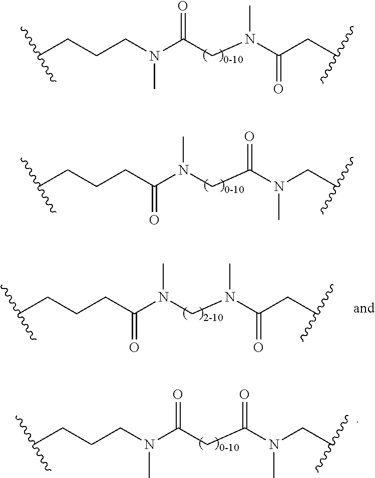

In certain embodiments, Linker is selected from:

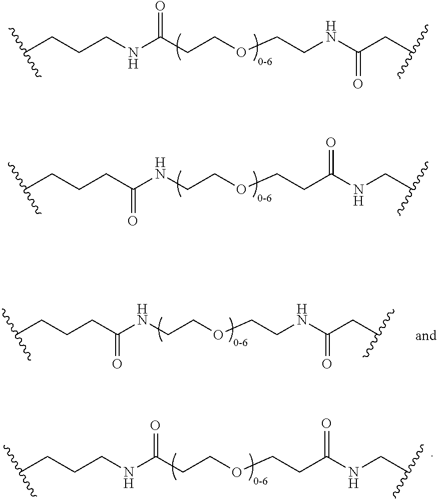

In certain embodiments, Linker is selected from:

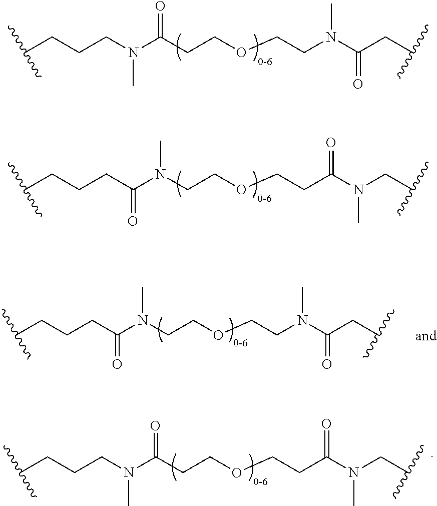

In certain embodiments, Linker is selected from:

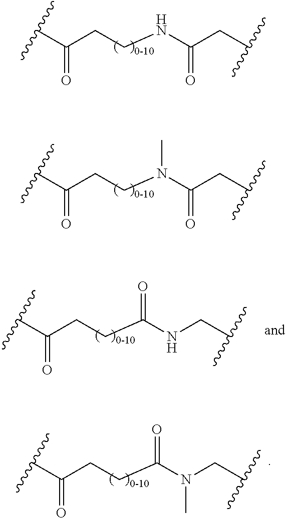

In certain embodiments, Linker is selected from:

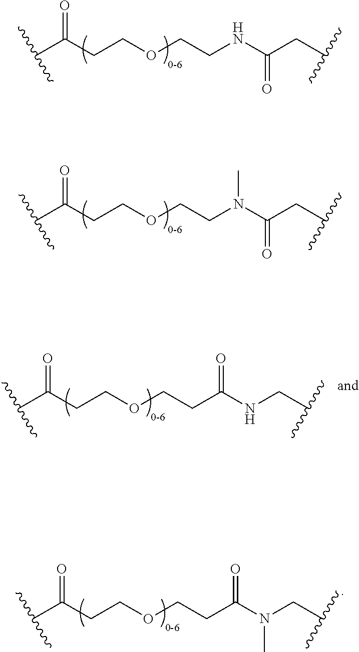

In certain embodiments, Linker is selected from:

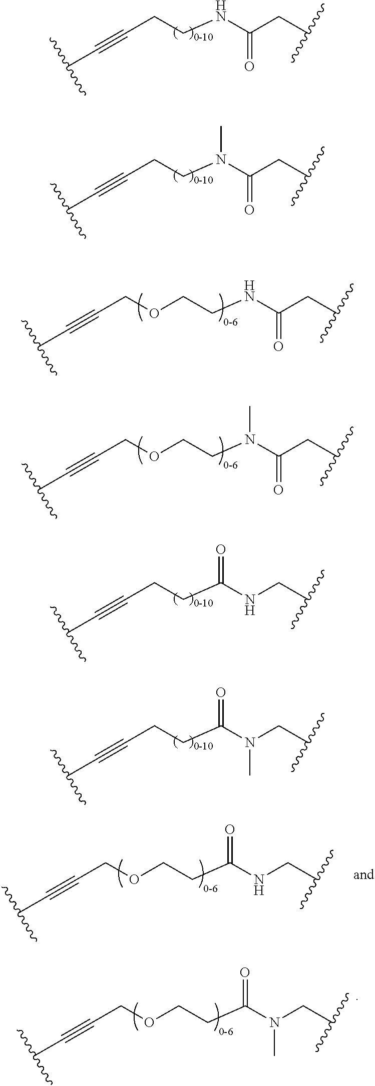

In certain embodiments, Linker is selected from:

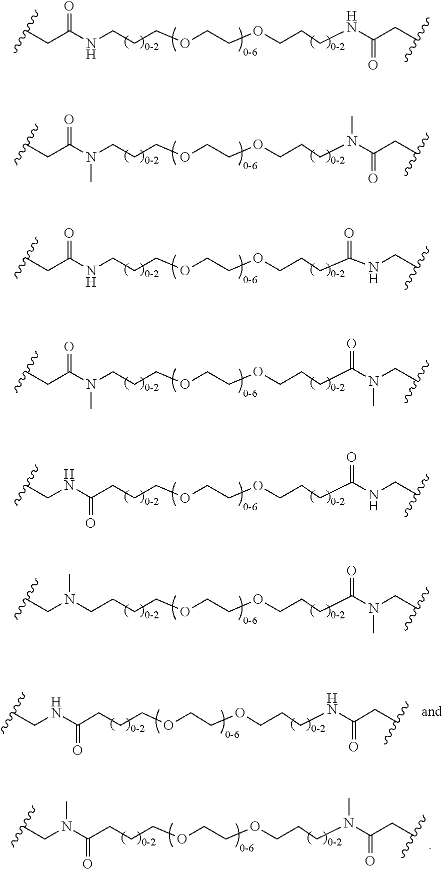

In certain embodiments, Linker is selected from:

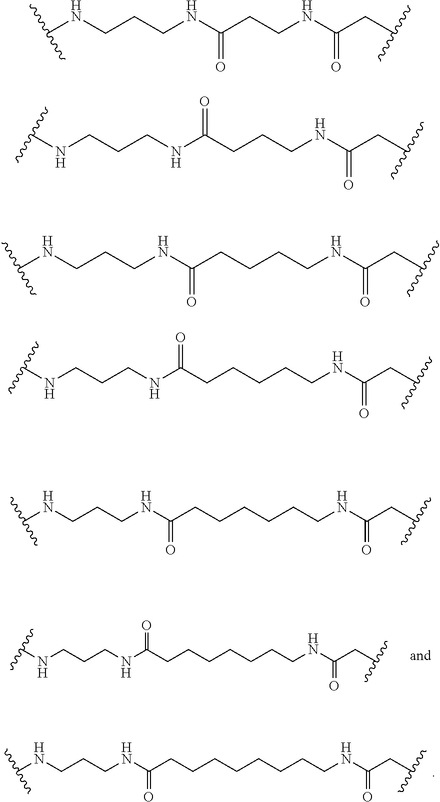

In certain embodiments, Linker is selected from:

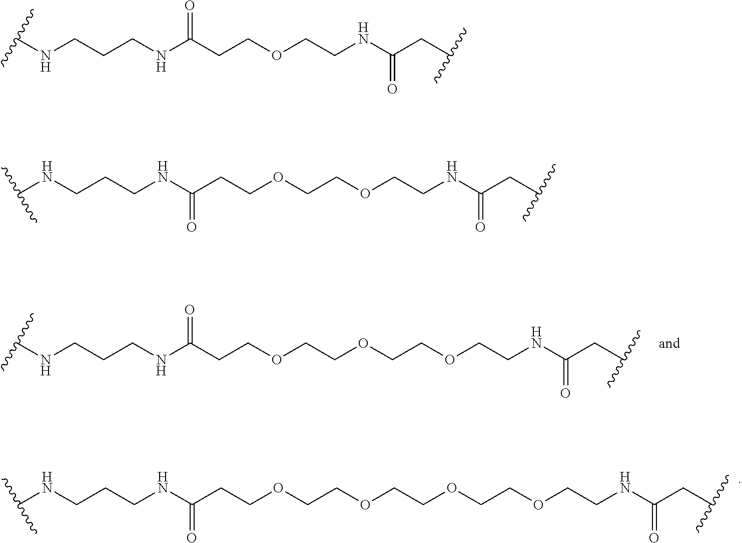 [SMILES: *CC(=O)NCCOCCC(=O)NCCCN* ; *CC(=O)NCCOCCOCCC(=O)NCCCN* ; *CC(=O)NCCOCCOCCOCCC(=O)NCCCN* ; *CC(=O)NCCOCCOCCOCCOCCC(=O)NCCCN*]

In certain embodiments, Linker is selected from:

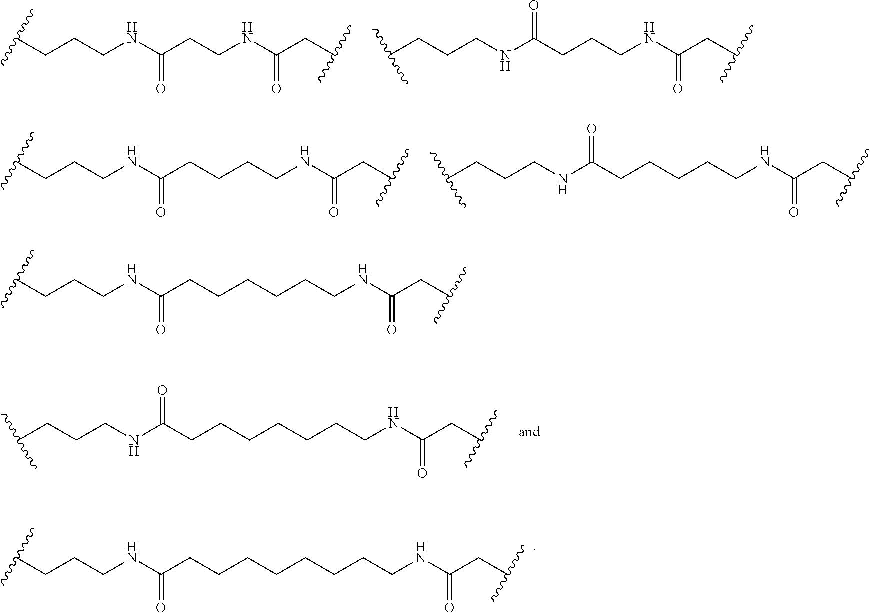

In certain embodiments, Linker is selected from:

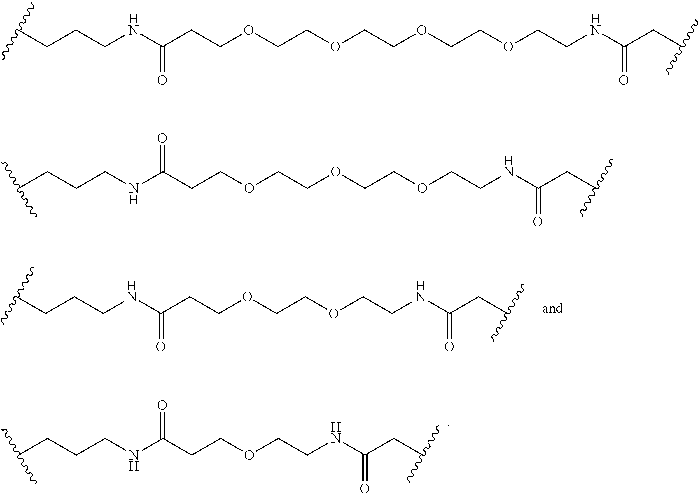 [SMILES: *CCCNC(=O)CCOCCOCCOCCOCCNC(=O)C* ; *CCCNC(=O)CCOCCOCCOCCNC(=O)C* ; *CCCNC(=O)CCOCCOCCNC(=O)C* ; *CCCNC(=O)CCOCCNC(=O)C*]

In certain embodiments, Linker is selected from:

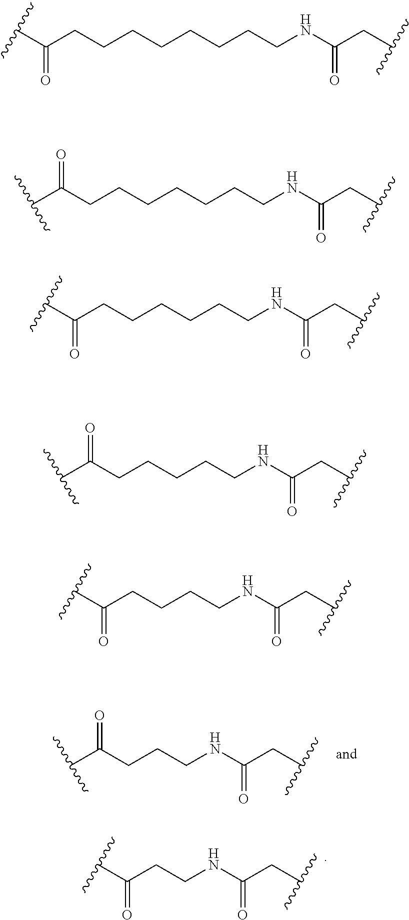

In certain embodiments, Linker is selected from:

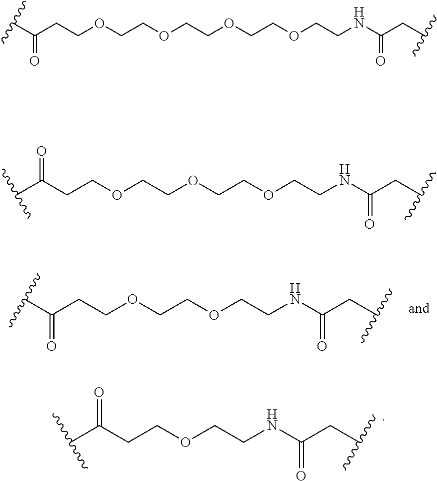 [SMILES: *CC(=O)NCCOCCOCCOCCOCCC(*)=O ; *CC(=O)NCCOCCOCCOCCC(*)=O ; *CC(=O)NCCOCCOCCC(*)=O ; *CC(=O)NCCOCCC(*)=O]

In certain embodiments, Linker is selected from:

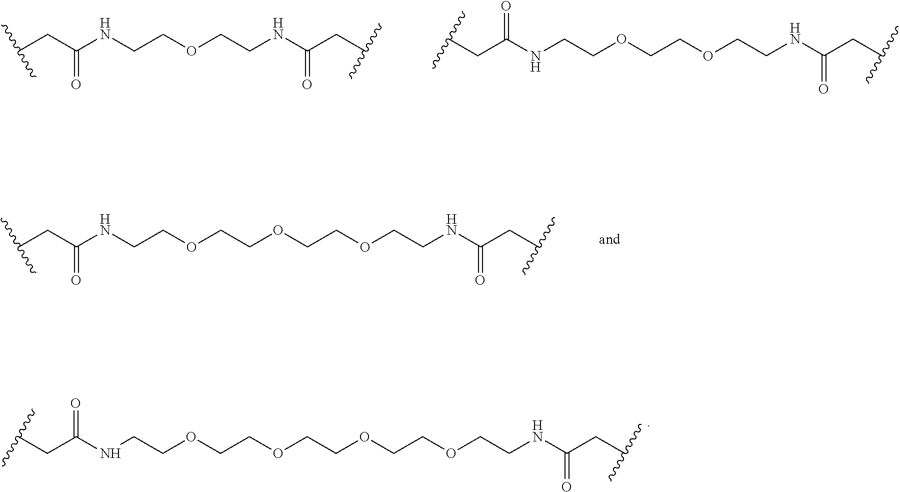 [SMILES: *CC(=O)NCCOCCNC(=O)C* ; *CC(=O)NCCOCCOCCNC(=O)C* ; *CC(=O)NCCOCCOCCOCCNC(=O)C* ; *CC(=O)NCCOCCOCCOCCOCCNC(=O)C*]

In certain embodiments, Linker is selected from:

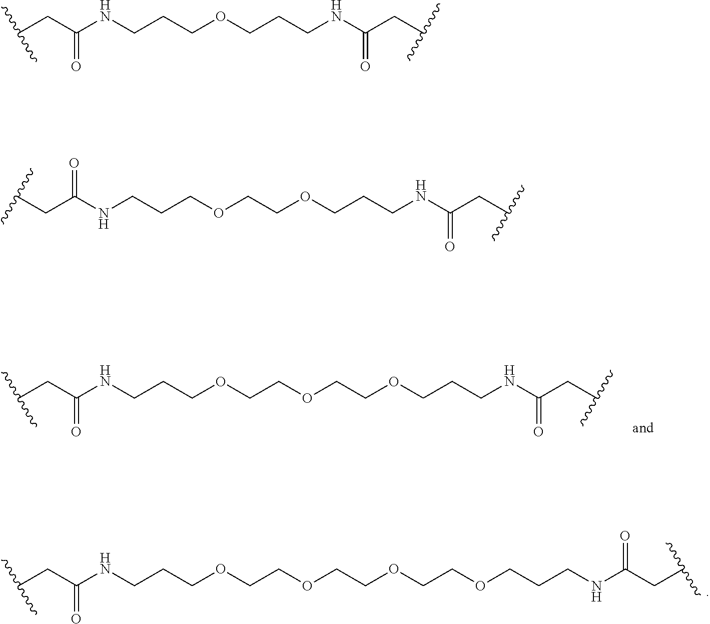 [SMILES: *CC(=O)NCCCOCCCNC(=O)C* ; *CC(=O)NCCCOCCOCCCNC(=O)C* ; *CC(=O)NCCCOCCOCCOCCCNC(=O)C* ; *CC(=O)NCCCOCCOCCOCCOCCCNC(=O)C*]

In certain embodiments, Linker is selected from:

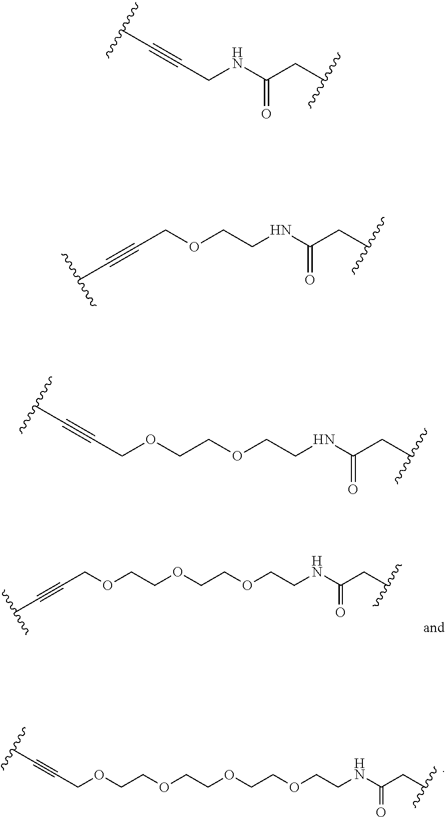 [SMILES: *C#CCNC(=O)C* ; *C#CCOCCNC(=O)C* ; *C#CCOCCOCCNC(=O)C* ; *C#CCOCCOCCOCCNC(=O)C* ; *C#CCOCCOCCOCCOCCNC(=O)C*]

In certain embodiments Linker is selected from:

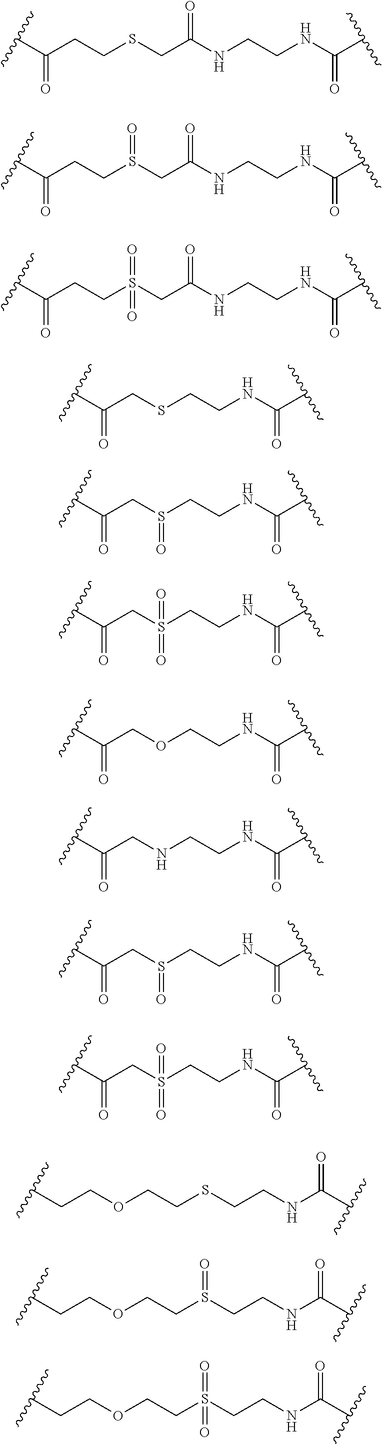

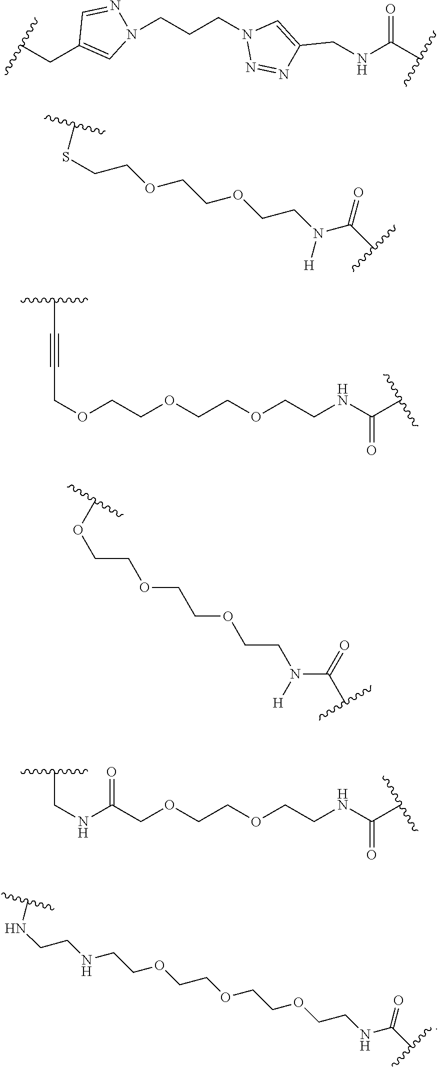 [SMILES: *Cc1cnn(CCCn2cc(CNC(*)=O)nn2)c1 ; *SCCOCCOCCNC(*)=O ; *C#CCOCCOCCOCCNC(*)=O ; *OCCOCCOCCNC(*)=O ; *CNC(=O)COCCOCCNC(*)=O ; *NCCNCCOCCOCCOCCNC(*)=O]

In certain embodiments, Linker, Linker-A, and/or Linker-B is selected from:

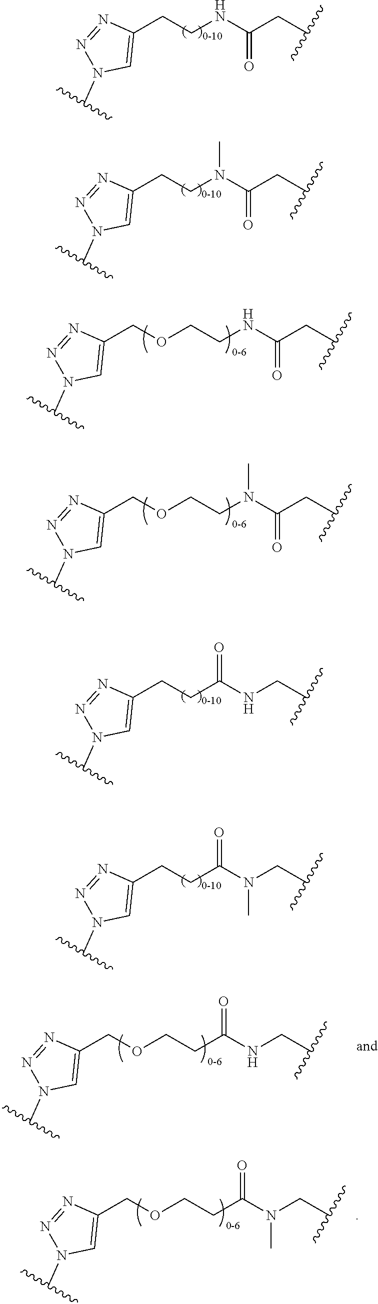

In certain embodiments, Linker, Linker-A, and/or Linker-B is selected from:

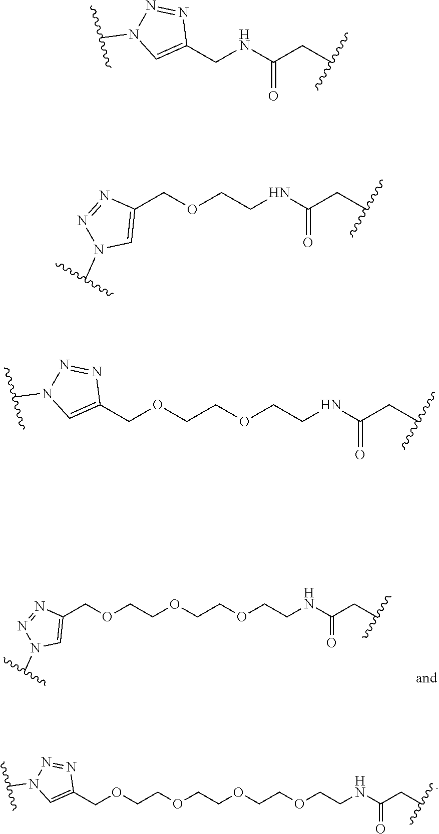 [SMILES: *CC(=O)NCc1cn(*)nn1 ; *CC(=O)NCCOCc1cn(*)nn1 ; *CC(=O)NCCOCCOCc1cn(*)nn1 ; *CC(=O)NCCOCCOCCOCc1cn(*)nn1 ; *CC(=O)NCCOCCOCCOCCOCc1cn(*)nn1]

In certain embodiments, Linker, Linker-A, and/or Linker-B is selected from:

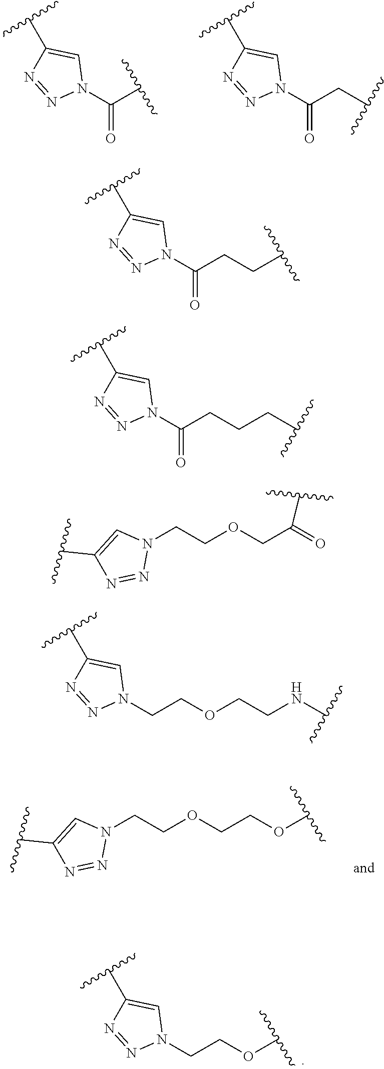

In certain embodiments, Linker-A is selected from:

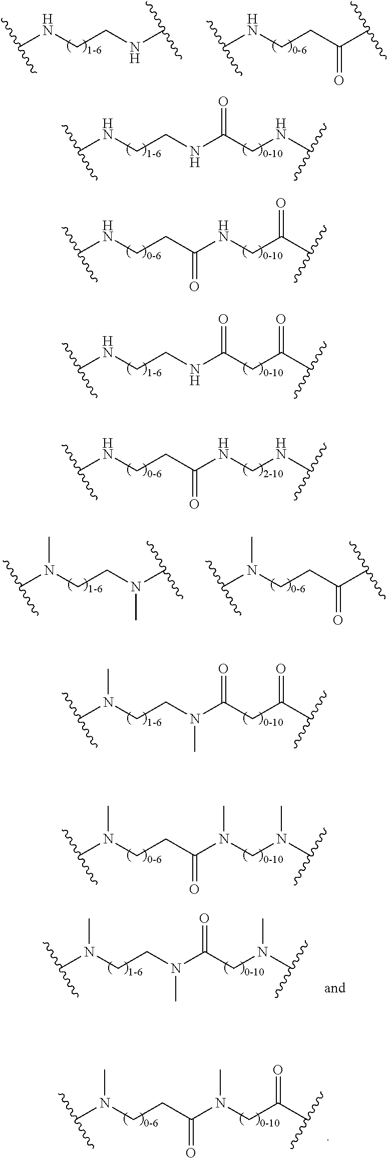

In certain embodiments, Linker-A is selected from:

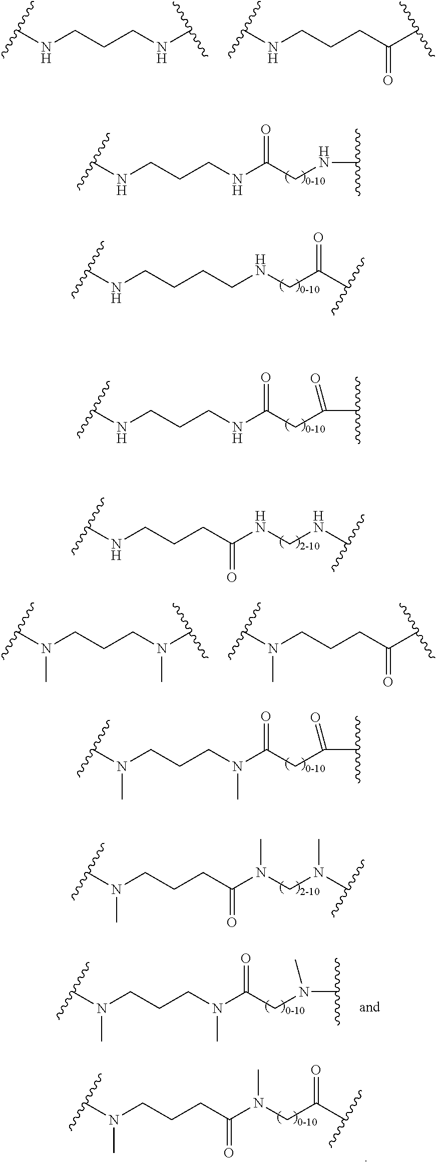

In certain embodiments, Linker-A is selected from:

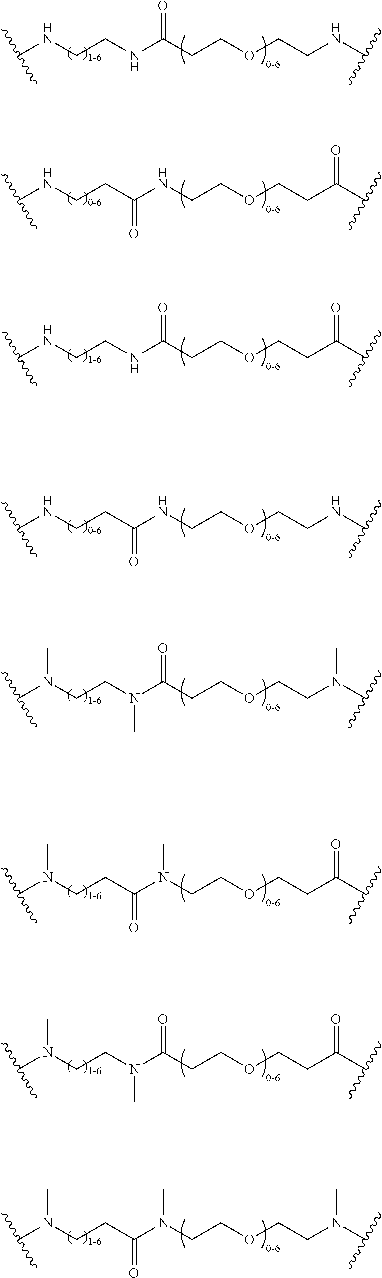

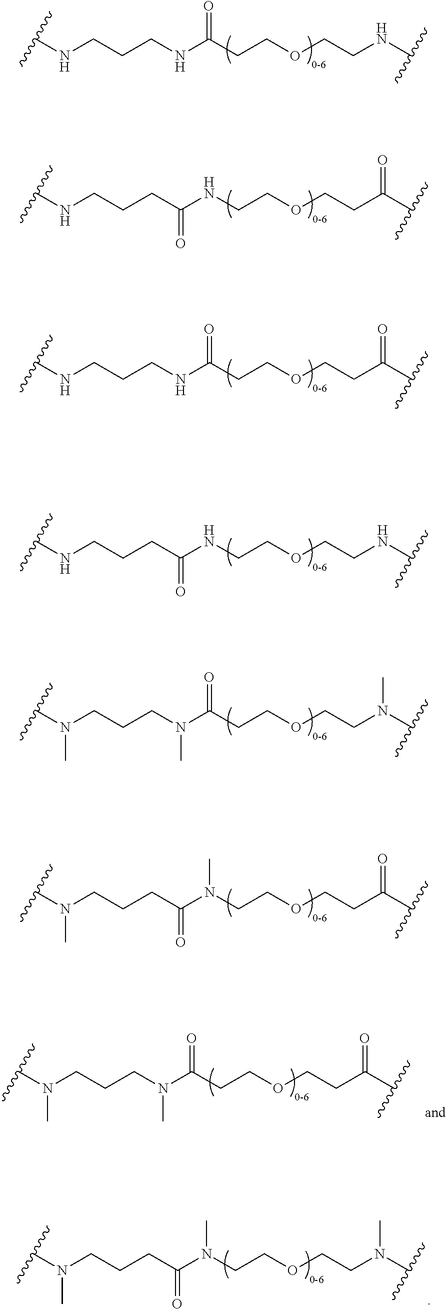

In certain embodiments, Linker-A is selected from:

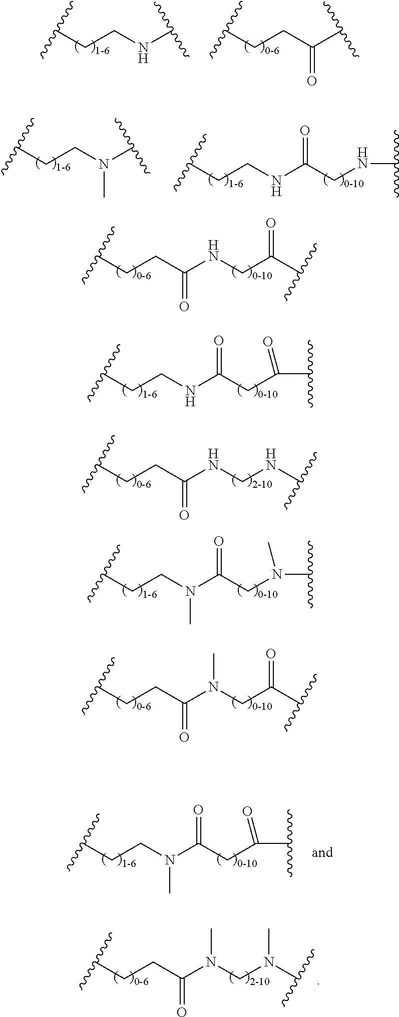

In certain embodiments, Linker-A is selected from:

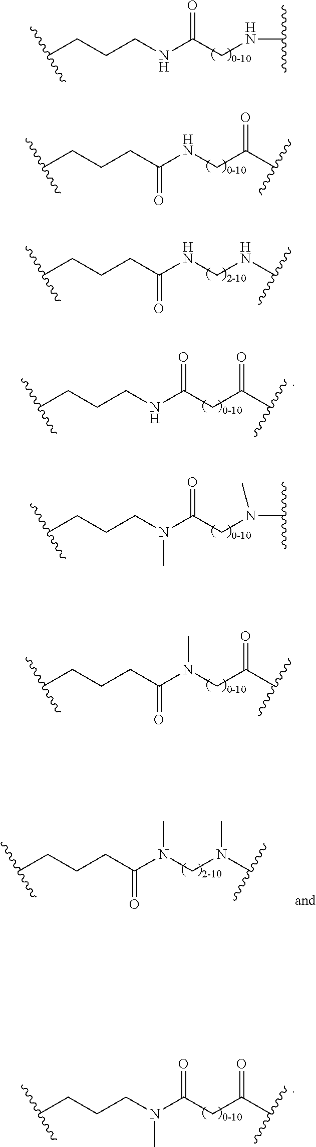

In certain embodiments, Linker-A is selected from:

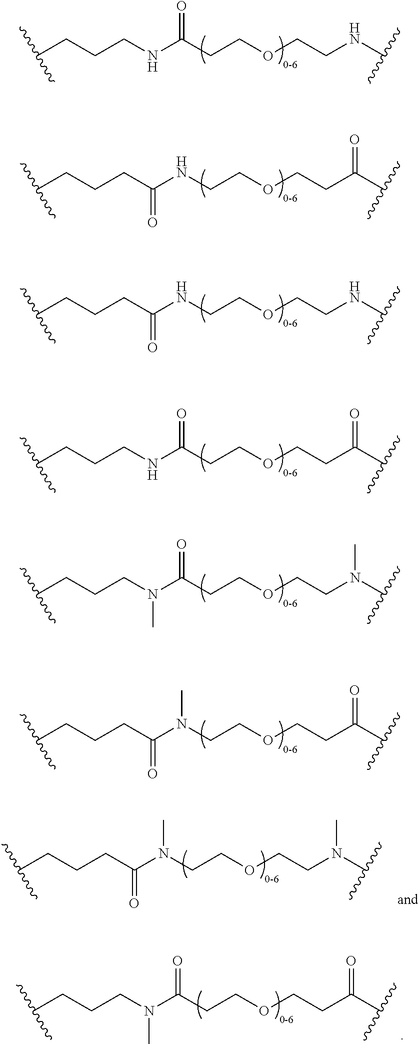

In certain embodiments, Linker-A is selected from:

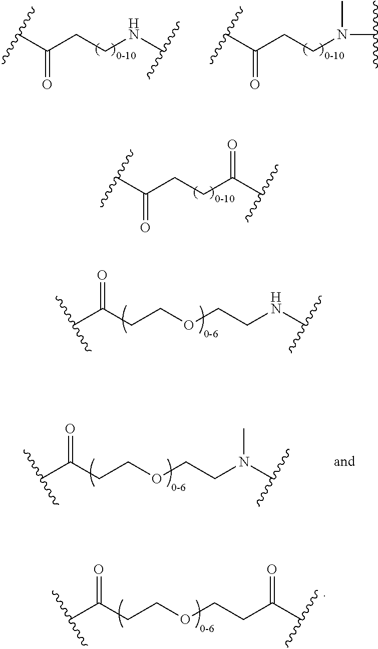

In certain embodiments, Linker-A is selected from:

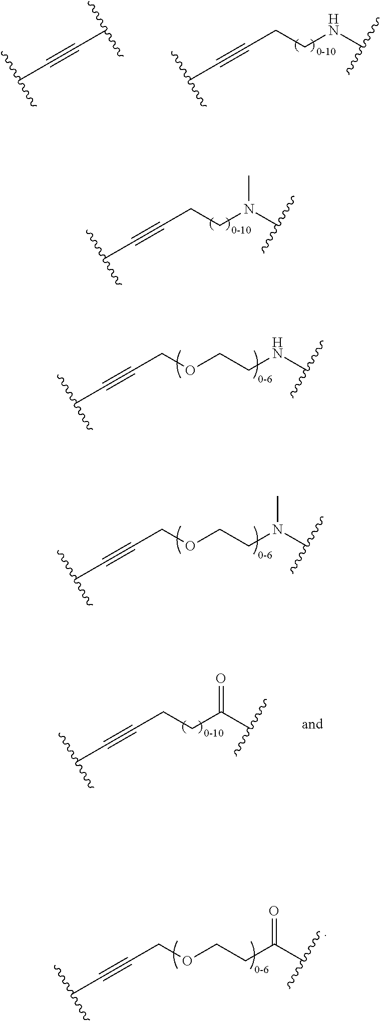

In certain embodiments, Linker-A is selected from:

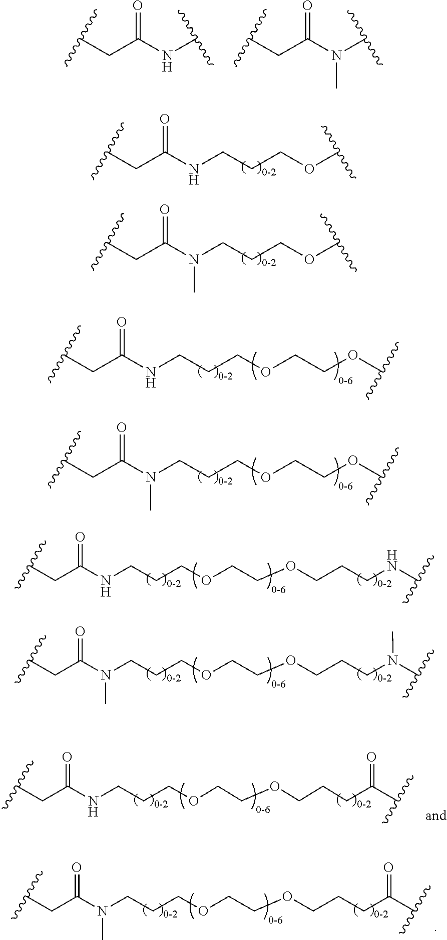

In certain embodiments, Linker-A is selected from:

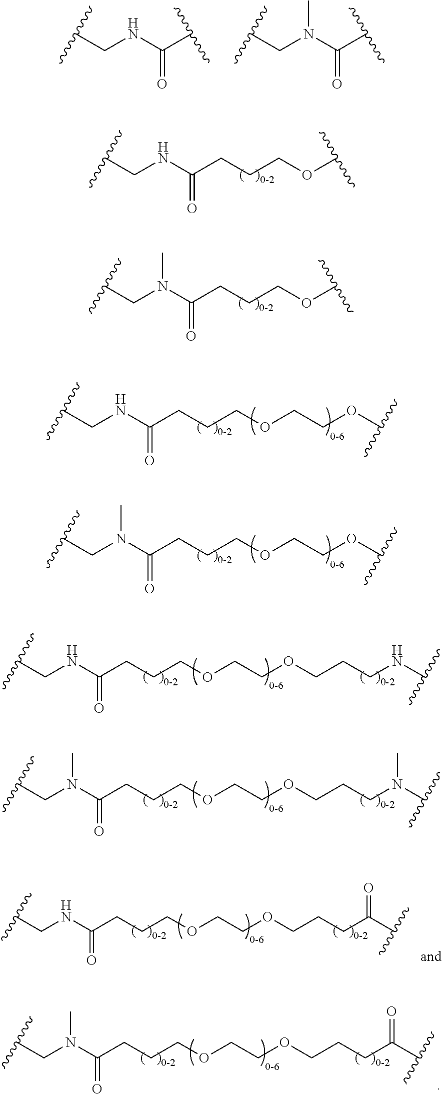

In certain embodiments, Linker-A is selected from:

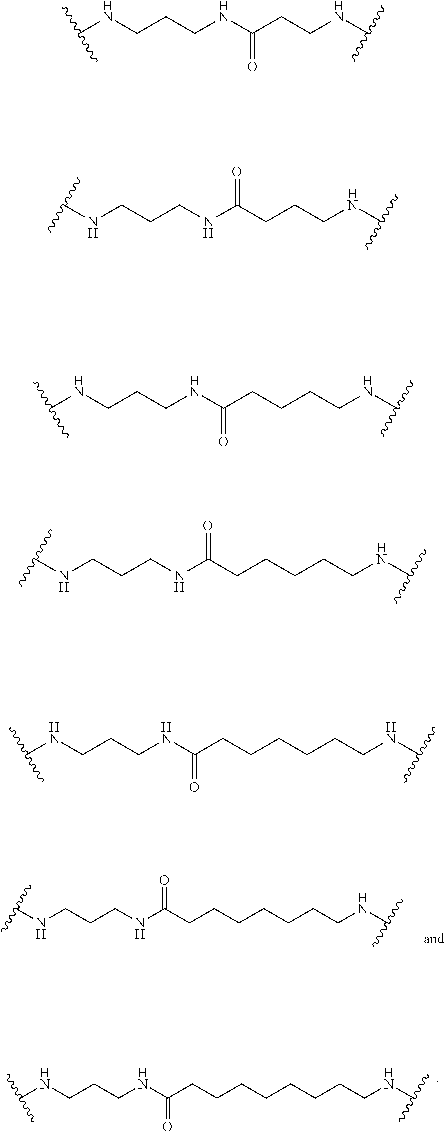

In certain embodiments, Linker-A is selected from:

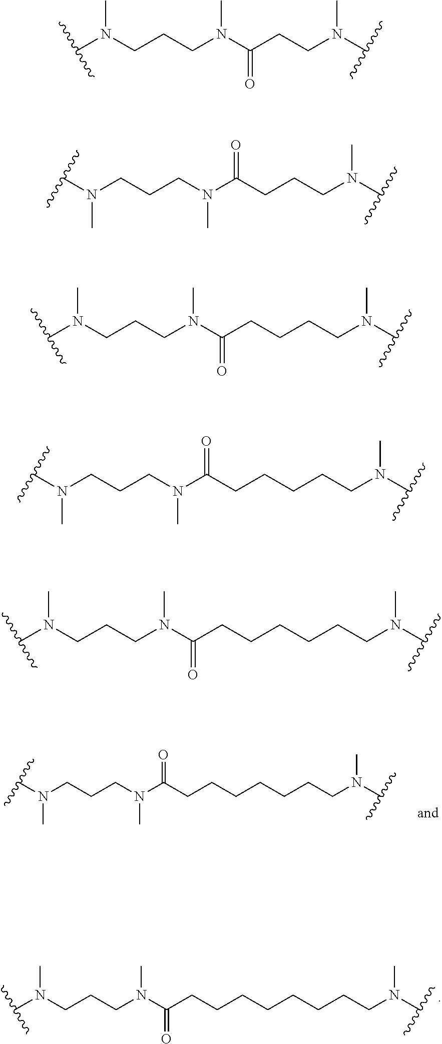

In certain embodiments, Linker-A is selected from:

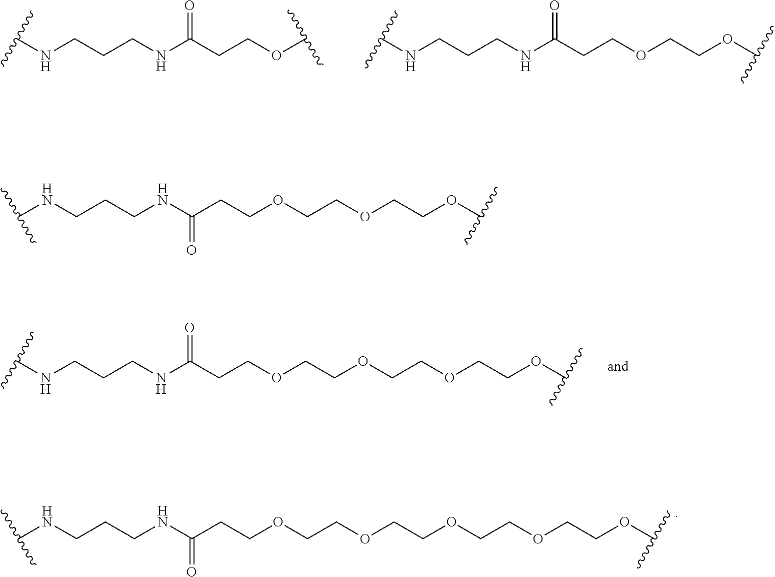 [SMILES: *NCCCNC(=O)CCO* ; *NCCCNC(=O)CCOCCO* ; *NCCCNC(=O)CCOCCOCCO* ; *NCCCNC(=O)CCOCCOCCOCCO* ; *NCCCNC(=O)CCOCCOCCOCCOCCO*]

In certain embodiments, Linker-A is selected from:

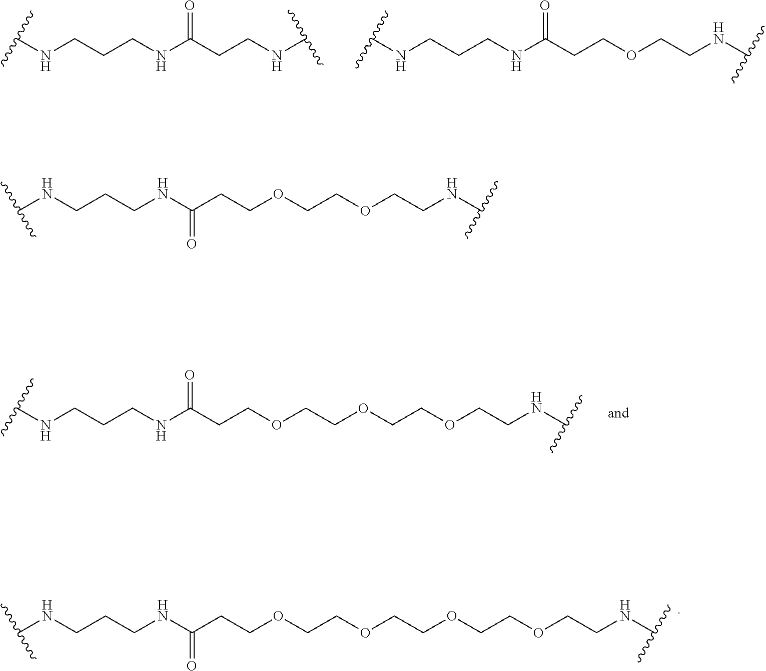 [SMILES: *NCCCNC(=O)CCN* ; *NCCCNC(=O)CCOCCN* ; *NCCCNC(=O)CCOCCOCCN* ; *NCCCNC(=O)CCOCCOCCOCCN* ; *NCCCNC(=O)CCOCCOCCOCCOCCN*]

In certain embodiments, Linker-A is selected from:

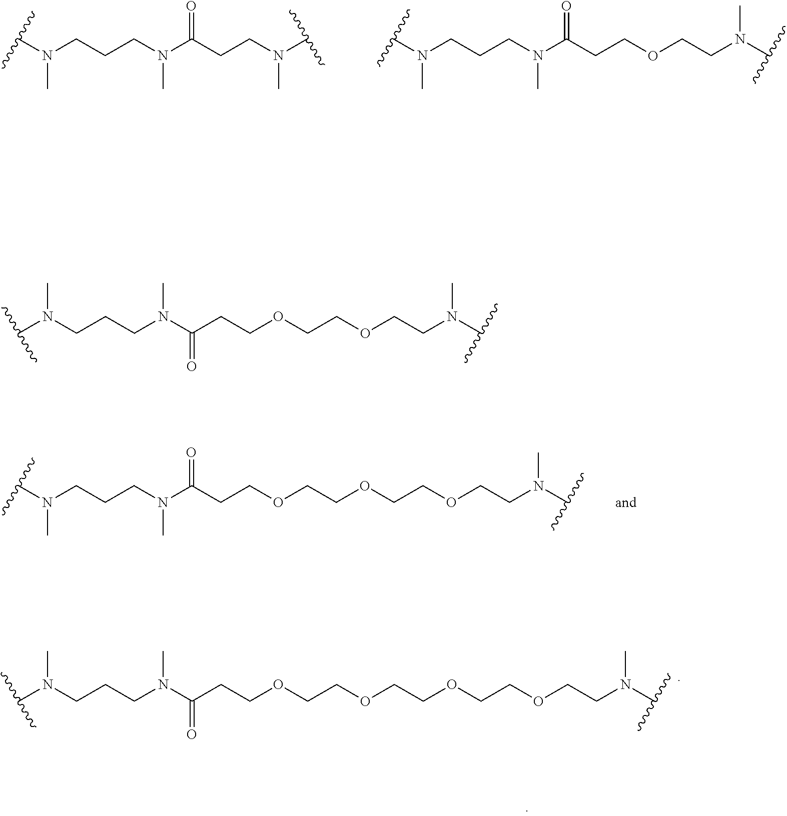 [SMILES: *N(C)CCCN(C)C(=O)CCN(*)C ; *N(C)CCCN(C)C(=O)CCOCCN(*)C ; *N(C)CCCN(C)C(=O)CCOCCOCCN(*)C ; *N(C)CCCN(C)C(=O)CCOCCOCCOCCN(*)C ; *N(C)CCCN(C)C(=O)CCOCCOCCOCCOCCN(*)C]

In certain embodiments, Linker-A is selected from:

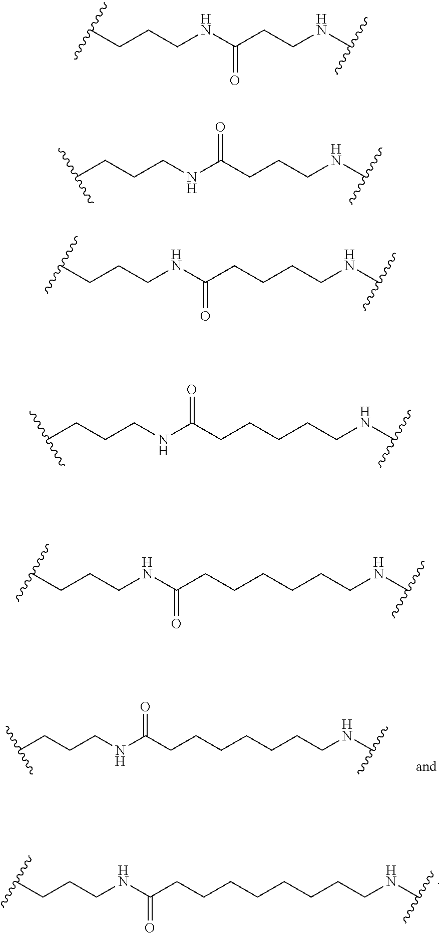

In certain embodiments, Linker-A is selected from:

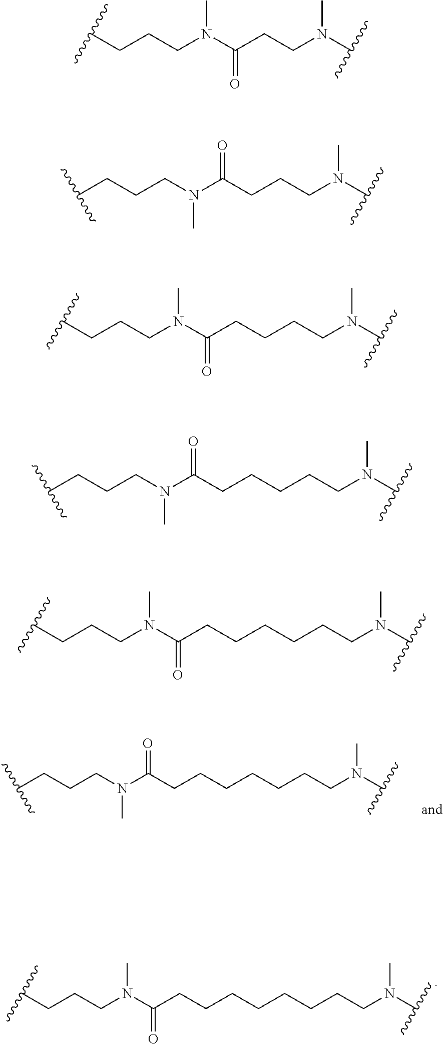

In certain embodiments, Linker-A is selected from:

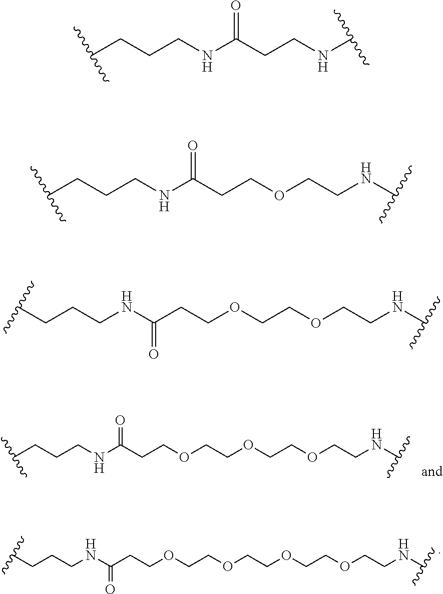 [SMILES: *CCCNC(=O)CCN* ; *CCCNC(=O)CCOCCN* ; *CCCNC(=O)CCOCCOCCN* ; *CCCNC(=O)CCOCCOCCOCCN* ; *CCCNC(=O)CCOCCOCCOCCOCCN*]

In certain embodiments, Linker-A is selected from:

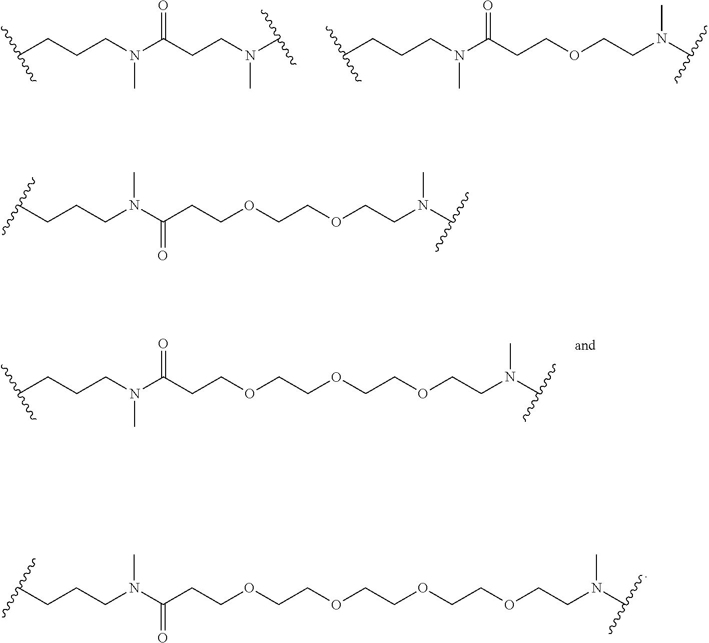 [SMILES: *CCCN(C)C(=O)CCN(*)C ; *CCCN(C)C(=O)CCOCCN(*)C ; *CCCN(C)C(=O)CCOCCOCCN(*)C ; *CCCN(C)C(=O)CCOCCOCCOCCN(*)C ; *CCCN(C)C(=O)CCOCCOCCOCCOCCN(*)C]

In certain embodiments, Linker-A is selected from:

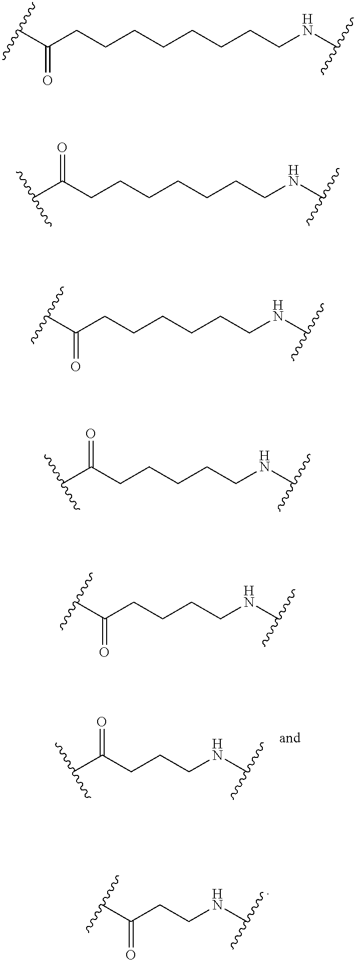

In certain embodiments, Linker-A is selected from:

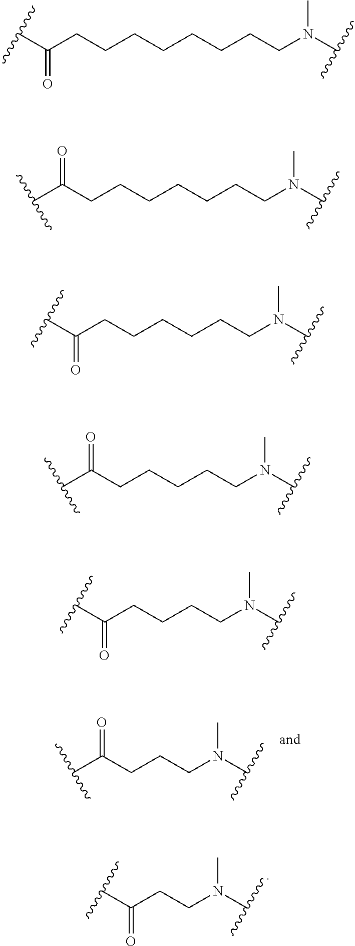

In certain embodiments, Linker-A is selected from:

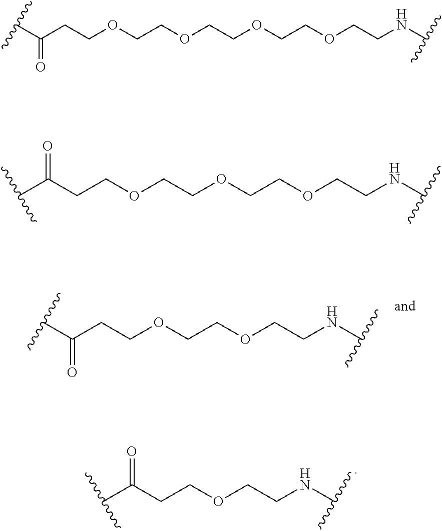 [SMILES: *NCCOCCOCCOCCOCCC(*)=O ; *NCCOCCOCCOCCC(*)=O ; *NCCOCCOCCC(*)=O ; *NCCOCCC(*)=O]

In certain embodiments, Linker-A is selected from:

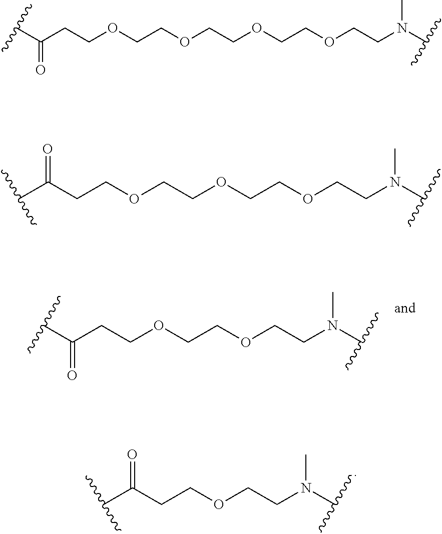 [SMILES: *C(=O)CCOCCOCCOCCOCCN(*)C ; *C(=O)CCOCCOCCOCCN(*)C ; *C(=O)CCOCCOCCN(*)C ; *C(=O)CCOCCN(*)C]

In certain embodiments, Linker-A is selected from:

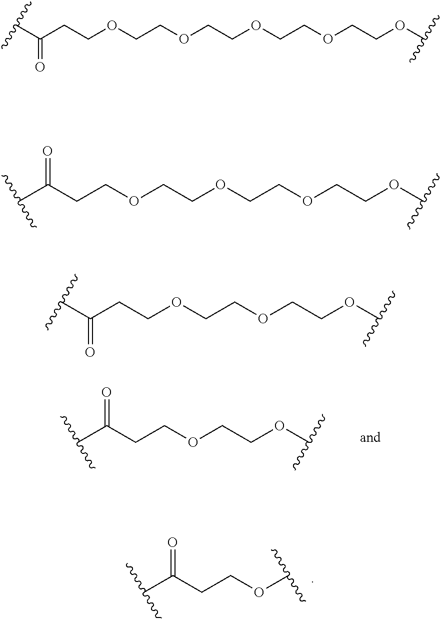

In certain embodiments, Linker-A is selected from:

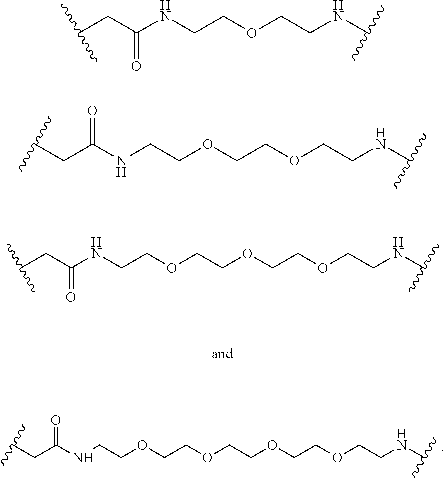 [SMILES: *CC(=O)NCCOCCN* ; *CC(=O)NCCOCCOCCN* ; *CC(=O)NCCOCCOCCOCCN* ; *CC(=O)NCCOCCOCCOCCOCCN*]

In certain embodiments, Linker-A is selected from:

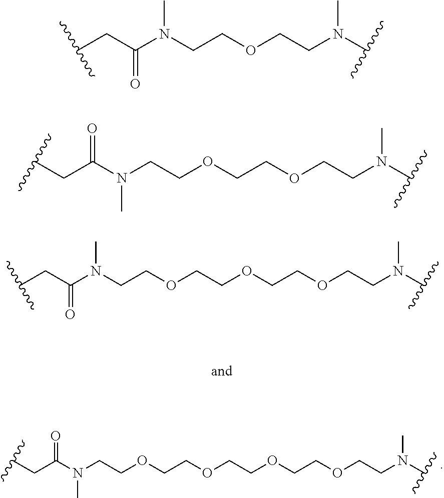

In certain embodiments, Linker-A is selected from:

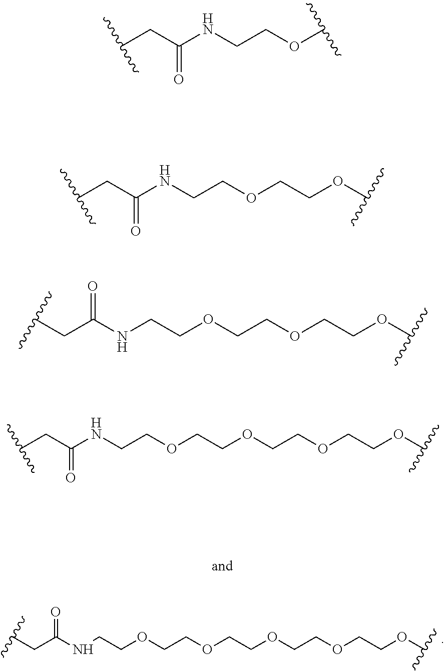 [SMILES: *CC(=O)NCCO* ; *CC(=O)NCCOCCO* ; *CC(=O)NCCOCCOCCO* ; *CC(=O)NCCOCCOCCOCCO* ; *CC(=O)NCCOCCOCCOCCOCCO*]

In certain embodiments, Linker-A is selected from:

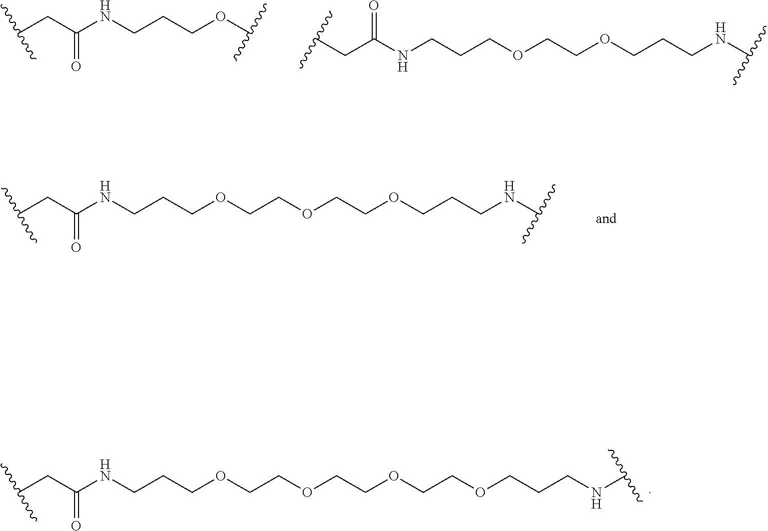 [SMILES: *CC(=O)NCCCO* ; *CC(=O)NCCCOCCOCCCN* ; *CC(=O)NCCCOCCOCCOCCCN* ; *CC(=O)NCCCOCCOCCOCCOCCCN*]

In certain embodiments, Linker-A is selected from:

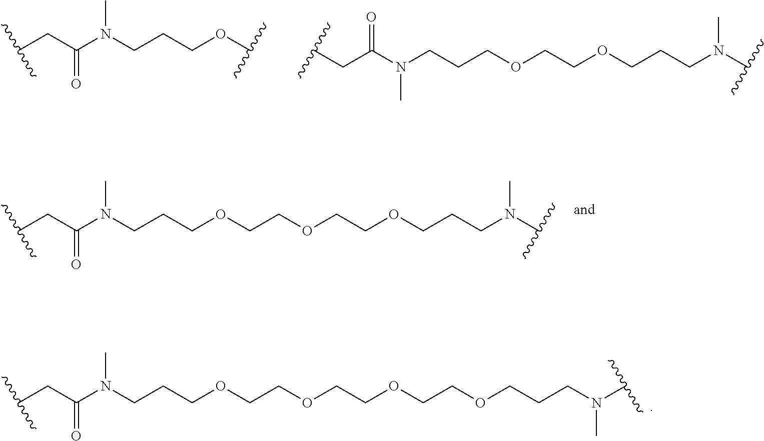 [SMILES: *CC(=O)N(C)CCCO* ; *CC(=O)N(C)CCCOCCOCCCN(*)C ; *CC(=O)N(C)CCCOCCOCCOCCCN(*)C ; *CC(=O)N(C)CCCOCCOCCOCCOCCCN(*)C]

In certain embodiments, Linker-A is selected from:

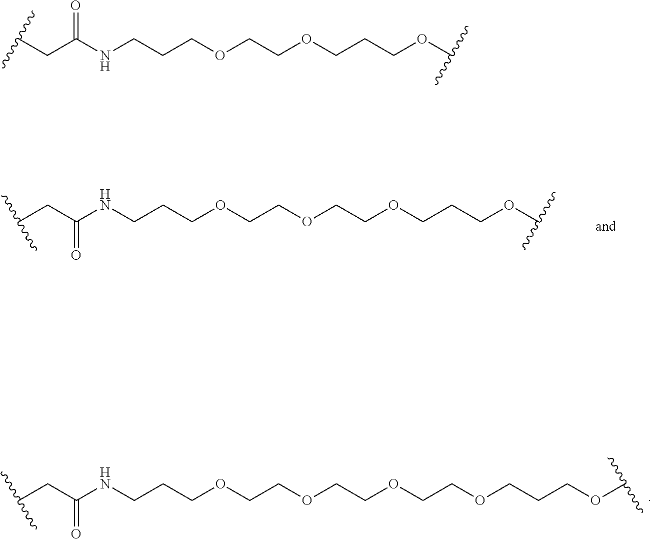 [SMILES: *CC(=O)NCCCOCCOCCCO* ; *CC(=O)NCCCOCCOCCOCCCO* ; *CC(=O)NCCCOCCOCCOCCOCCCO*]

In certain embodiments, Linker-A is selected from:

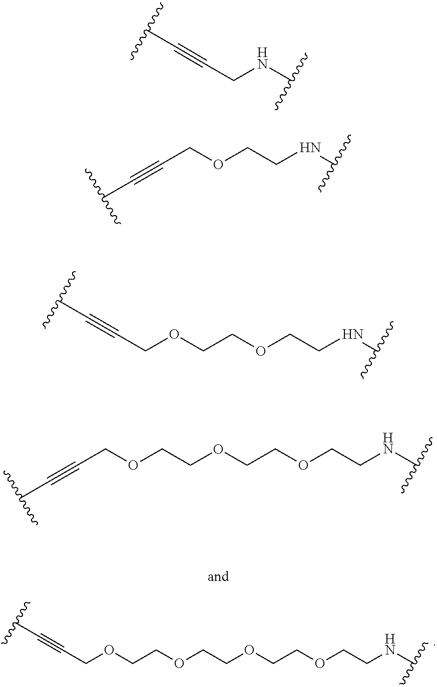 [SMILES: *C#CCN* ; *C#CCOCCN* ; *C#CCOCCOCCN* ; *C#CCOCCOCCOCCN* ; *C#CCOCCOCCOCCOCCN*]

In certain embodiments, Linker-A is selected from:

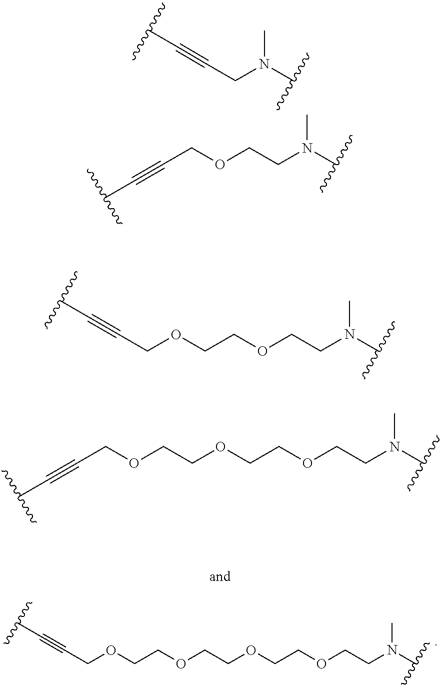

In certain embodiments, Linker-A is selected from:

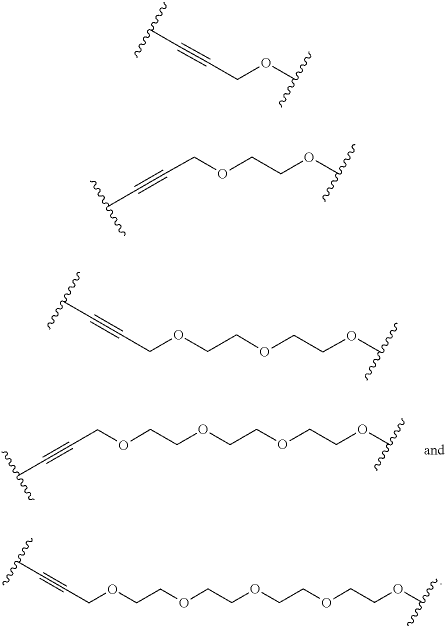 [SMILES: *C#CCO* ; *C#CCOCCO* ; *C#CCOCCOCCO* ; *C#CCOCCOCCOCCO* ; *C#CCOCCOCCOCCOCCO*]

In certain embodiments, Linker-B is selected from:

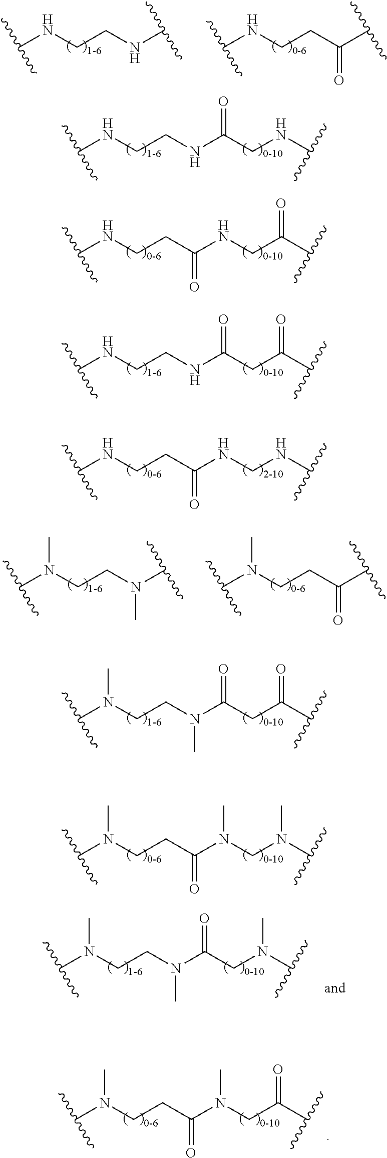

In certain embodiments, Linker-B is selected from:

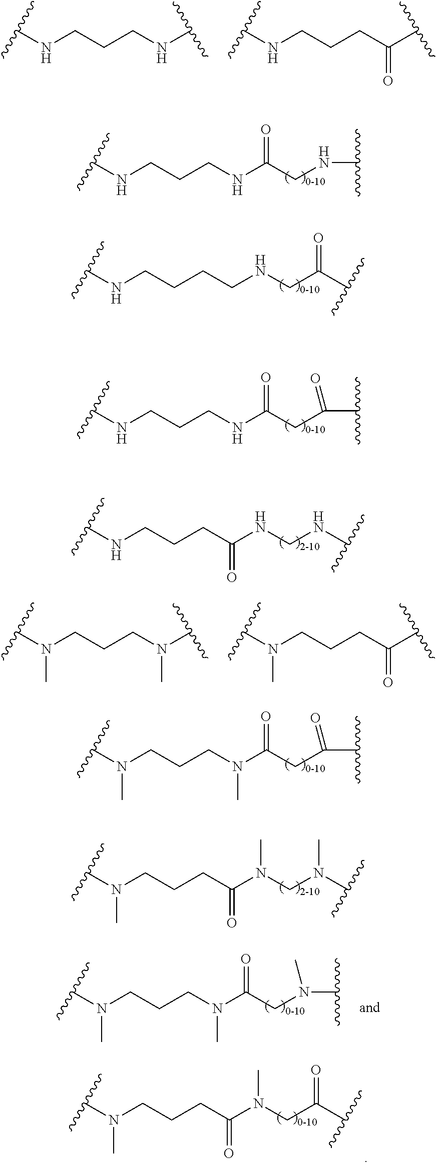

In certain embodiments, Linker-B is selected from:

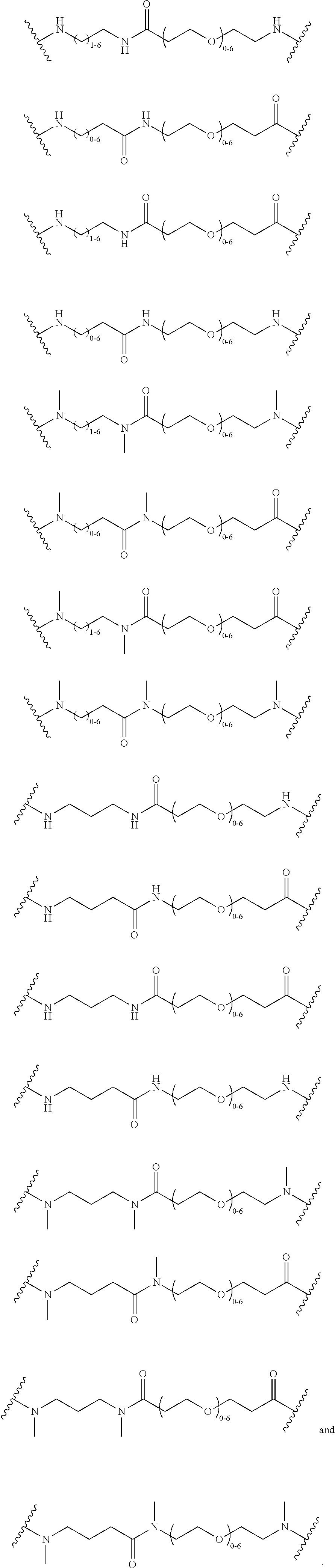

In certain embodiments, Linker-B is selected from:

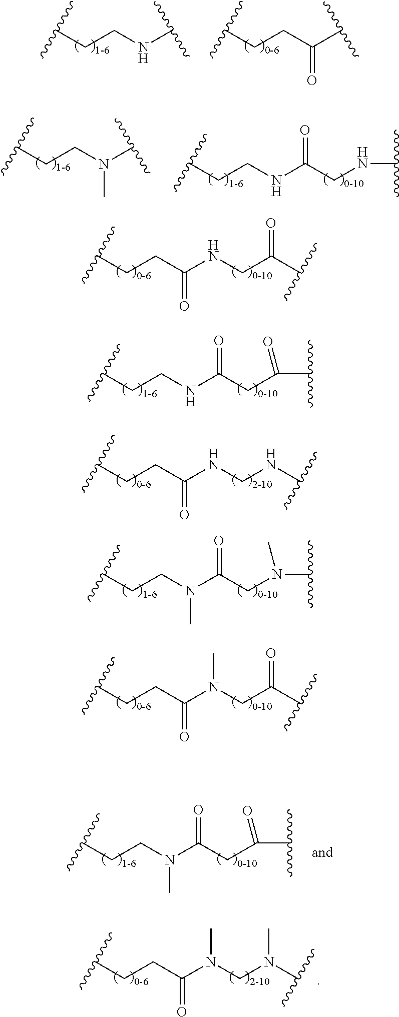

In certain embodiments, Linker-B is selected from:

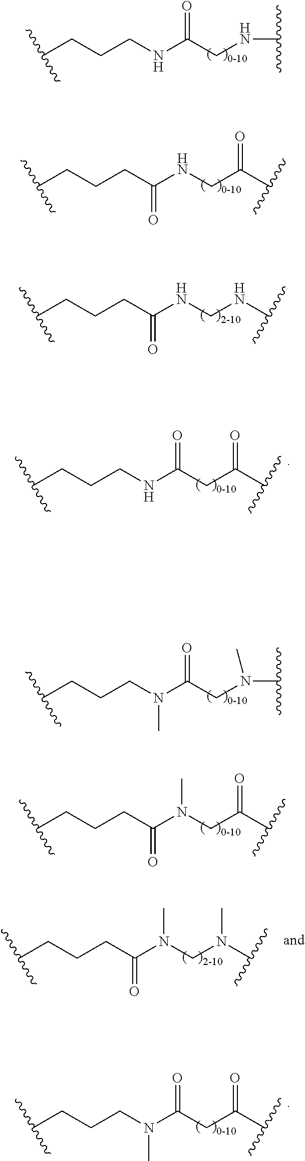

In certain embodiments, Linker-B is selected from:

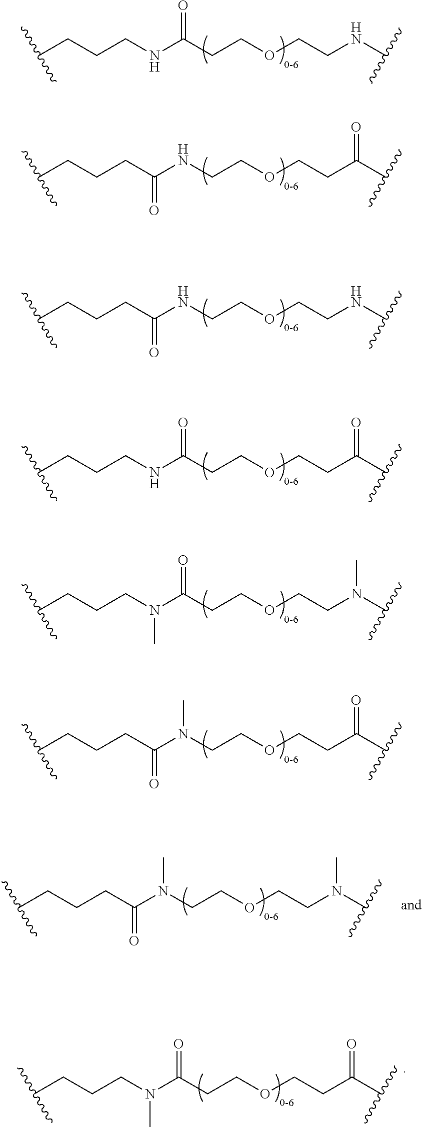

In certain embodiments, Linker-B is selected from:

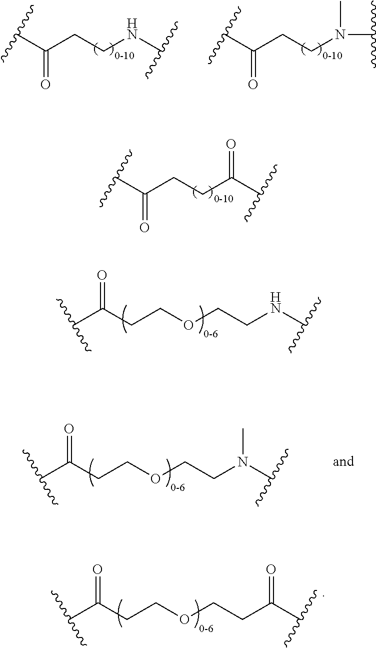

In certain embodiments, Linker-B is selected from:

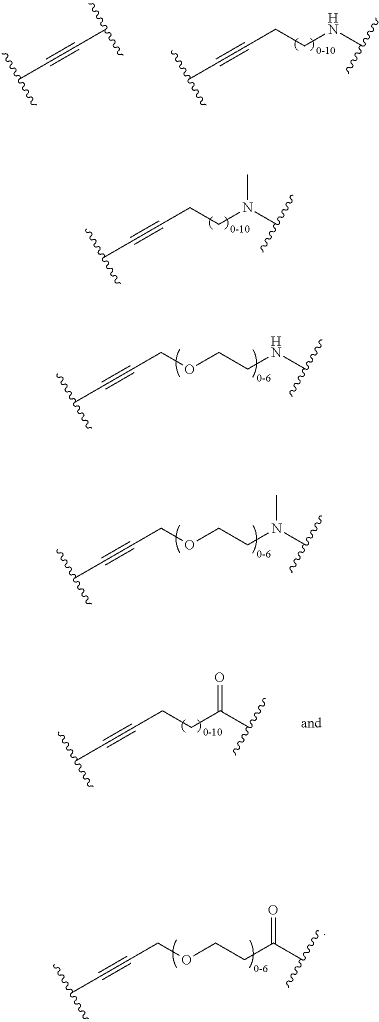

In certain embodiments, Linker-B is selected from:

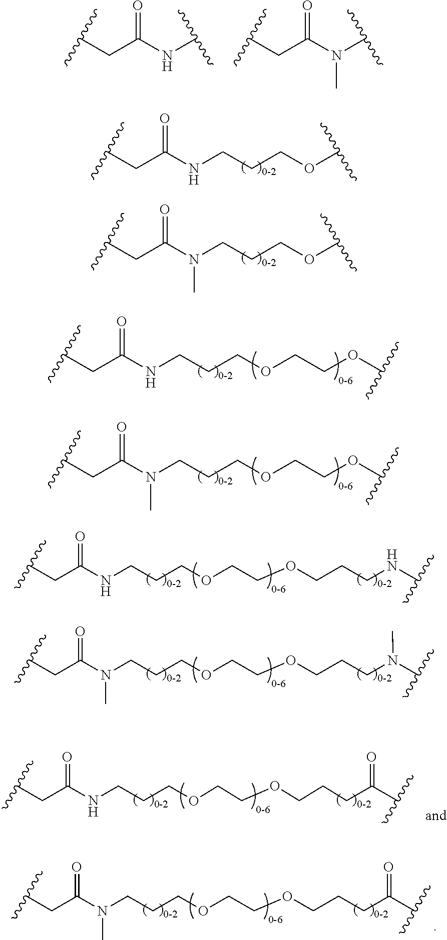

In certain embodiments, Linker-B is selected from:

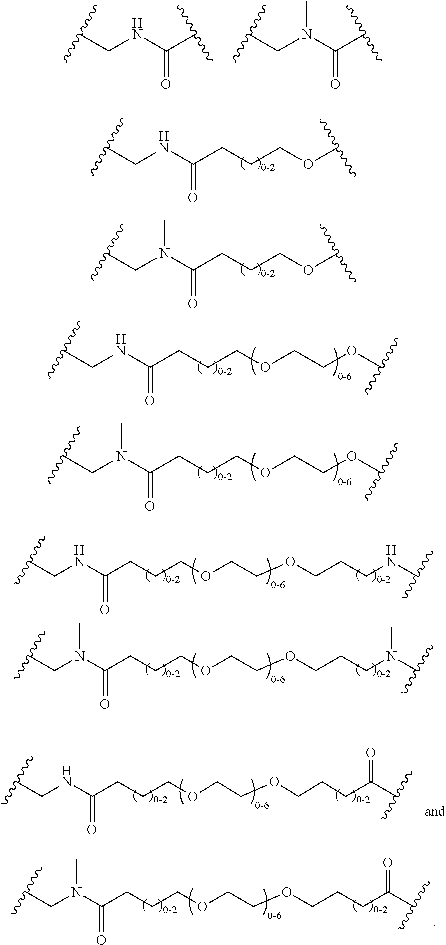

In certain embodiments, Linker-B is selected from:

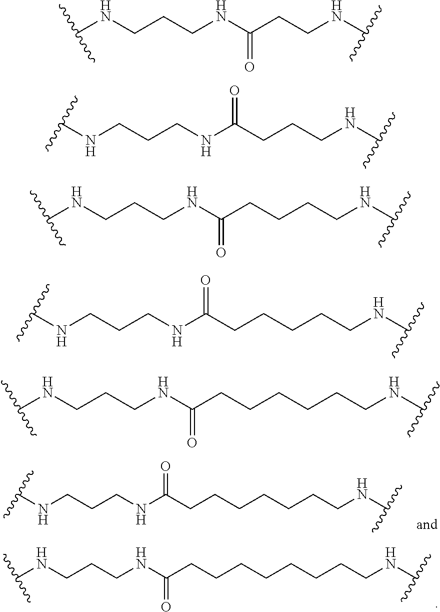

In certain embodiments, Linker-B is selected from:

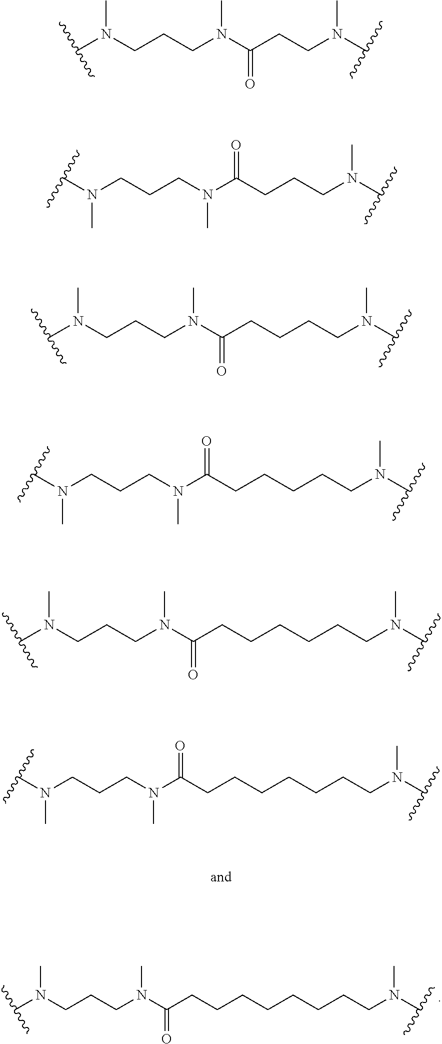

In certain embodiments, Linker-B is selected from:

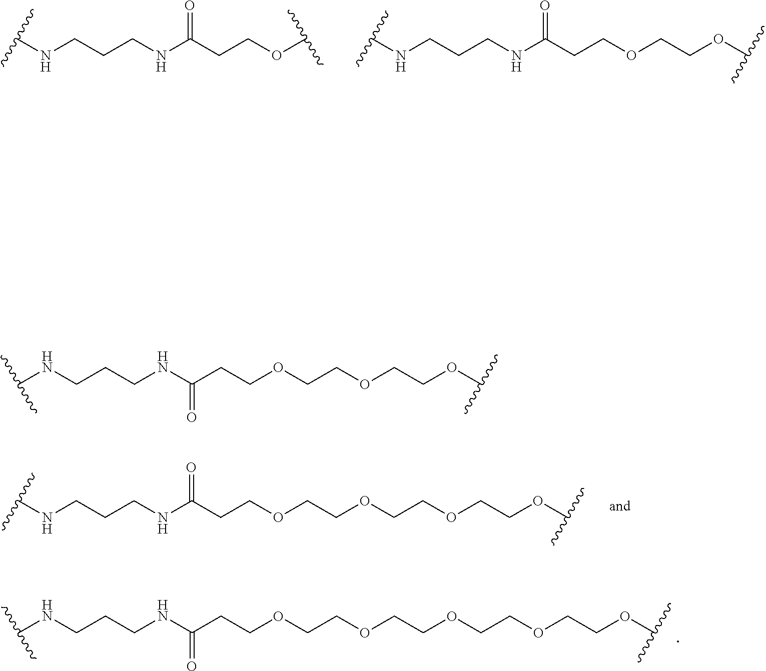 [SMILES: *NCCCNC(=O)CCO* ; *NCCCNC(=O)CCOCCO* ; *NCCCNC(=O)CCOCCOCCO* ; *NCCCNC(=O)CCOCCOCCOCCO* ; *NCCCNC(=O)CCOCCOCCOCCOCCO*]

In certain embodiments, Linker-B is selected from:

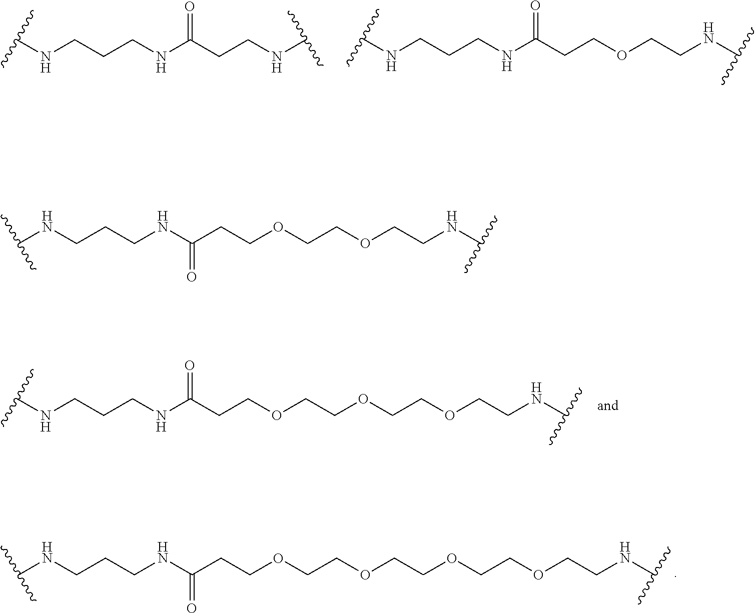 [SMILES: *NCCCNC(=O)CCN* ; *NCCCNC(=O)CCOCCN* ; *NCCCNC(=O)CCOCCOCCN* ; *NCCCNC(=O)CCOCCOCCOCCN* ; *NCCCNC(=O)CCOCCOCCOCCOCCN*]

In certain embodiments, Linker-B is selected from:

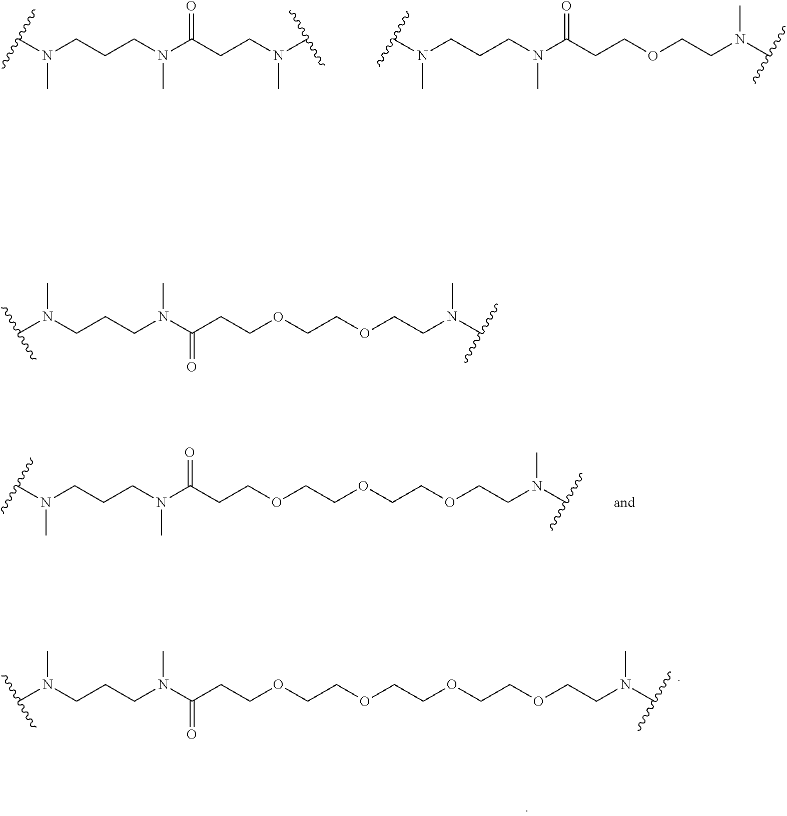 [SMILES: *N(C)CCCN(C)C(=O)CCN(*)C ; *N(C)CCCN(C)C(=O)CCOCCN(*)C ; *N(C)CCCN(C)C(=O)CCOCCOCCN(*)C ; *N(C)CCCN(C)C(=O)CCOCCOCCOCCN(*)C ; *N(C)CCCN(C)C(=O)CCOCCOCCOCCOCCN(*)C]

In certain embodiments, Linker-B is selected from:

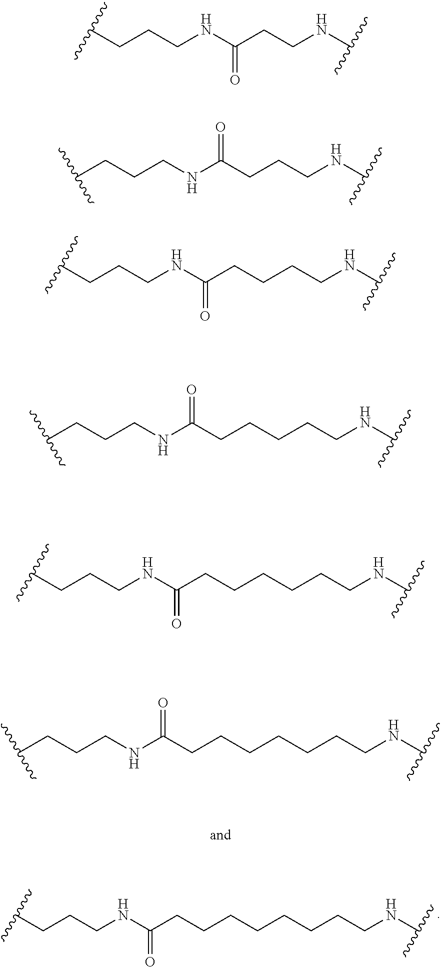

In certain embodiments, Linker-B is selected from:

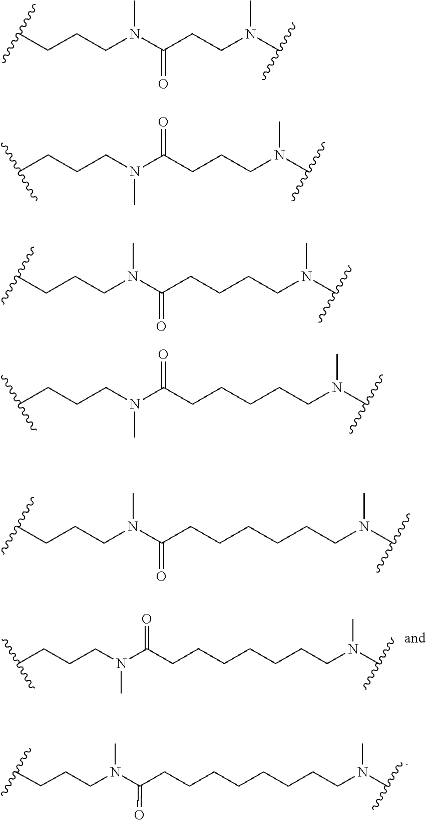

In certain embodiments, Linker-B is selected from:

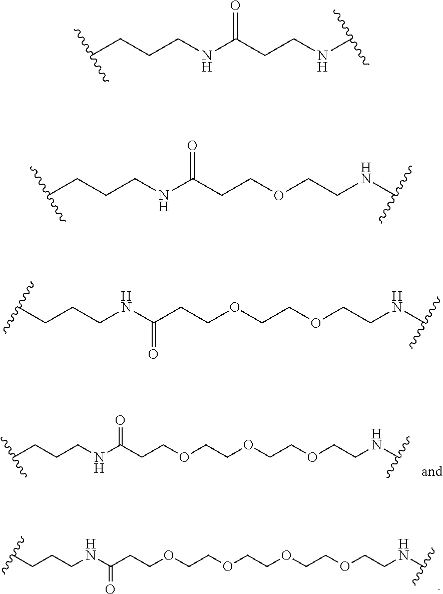 [SMILES: *CCCNC(=O)CCN* ; *CCCNC(=O)CCOCCN* ; *CCCNC(=O)CCOCCOCCN* ; *CCCNC(=O)CCOCCOCCOCCN* ; *CCCNC(=O)CCOCCOCCOCCOCCN*]

In certain embodiments, Linker-B is selected from:

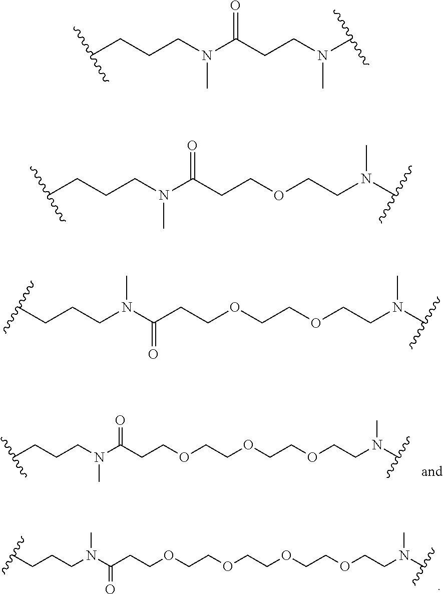 [SMILES: *CCCN(C)C(=O)CCN(*)C ; *CCCN(C)C(=O)CCOCCN(*)C ; *CCCN(C)C(=O)CCOCCOCCN(*)C ; *CCCN(C)C(=O)CCOCCOCCOCCN(*)C ; *CCCN(C)C(=O)CCOCCOCCOCCOCCN(*)C]

In certain embodiments, Linker-B is selected from:

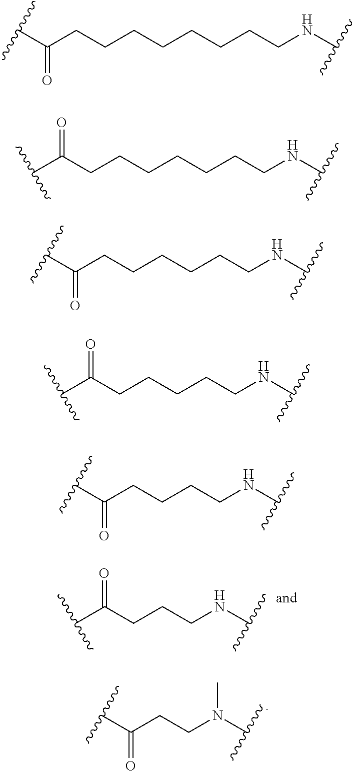

In certain embodiments, Linker-B is selected from:

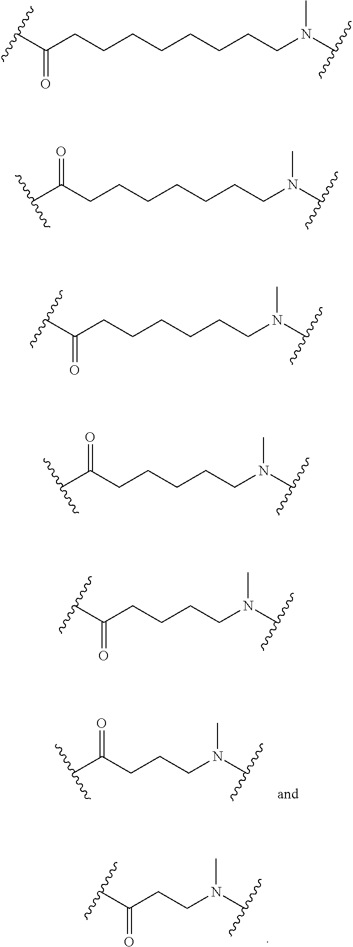

In certain embodiments, Linker-B is selected from:

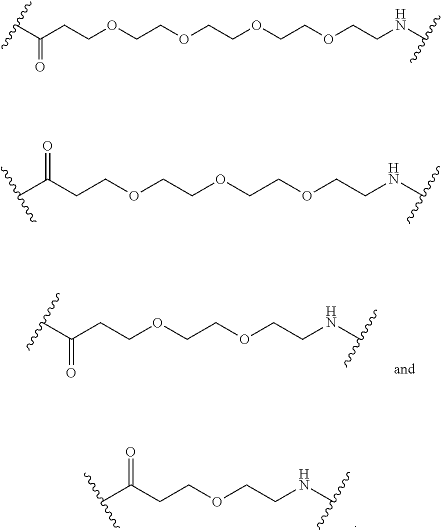 [SMILES: *NCCOCCOCCOCCOCCC(*)=O ; *NCCOCCOCCOCCC(*)=O ; *NCCOCCOCCC(*)=O ; *NCCOCCC(*)=O]

In certain embodiments, Linker-B is selected from:

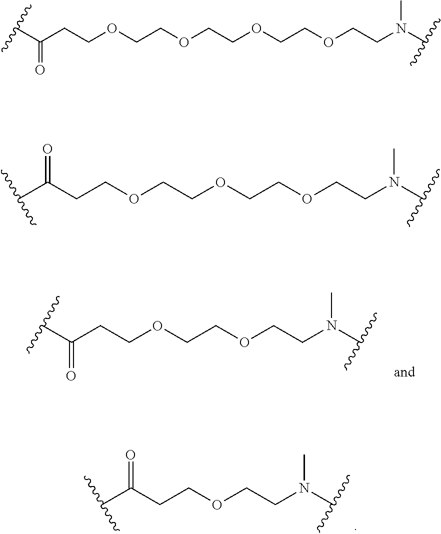 [SMILES: *C(=O)CCOCCOCCOCCOCCN(*)C ; *C(=O)CCOCCOCCOCCN(*)C ; *C(=O)CCOCCOCCN(*)C ; *C(=O)CCOCCN(*)C]

In certain embodiments, Linker-B is selected from:

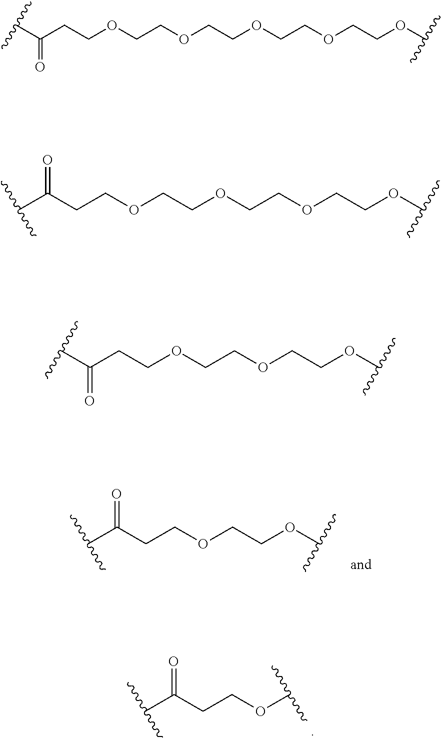 [SMILES: *OCCOCCOCCOCCOCCC(*)=O ; *OCCOCCOCCOCCC(*)=O ; *OCCOCCOCCC(*)=O ; *OCCOCCC(*)=O ; *OCCC(*)=O]

In certain embodiments, Linker-B is selected from:

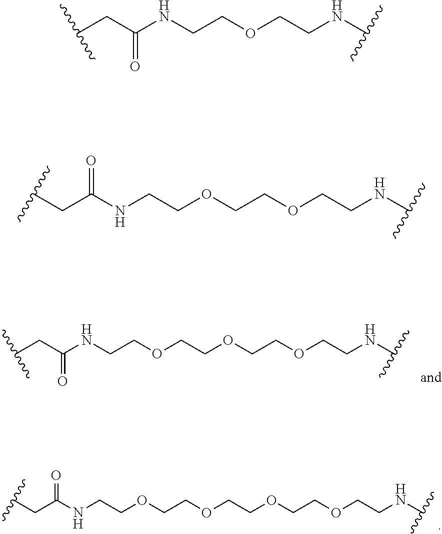 [SMILES: *CC(=O)NCCOCCN* ; *CC(=O)NCCOCCOCCN* ; *CC(=O)NCCOCCOCCOCCN* ; *CC(=O)NCCOCCOCCOCCOCCN*]

In certain embodiments, Linker-B is selected from:

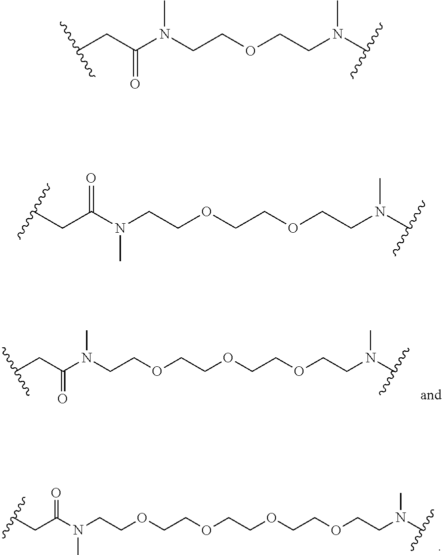 [SMILES: *CC(=O)N(C)CCOCCN(*)C ; *CC(=O)N(C)CCOCCOCCN(*)C ; *CC(=O)N(C)CCOCCOCCOCCN(*)C ; *CC(=O)N(C)CCOCCOCCOCCOCCN(*)C]

In certain embodiments, Linker-B is selected from:

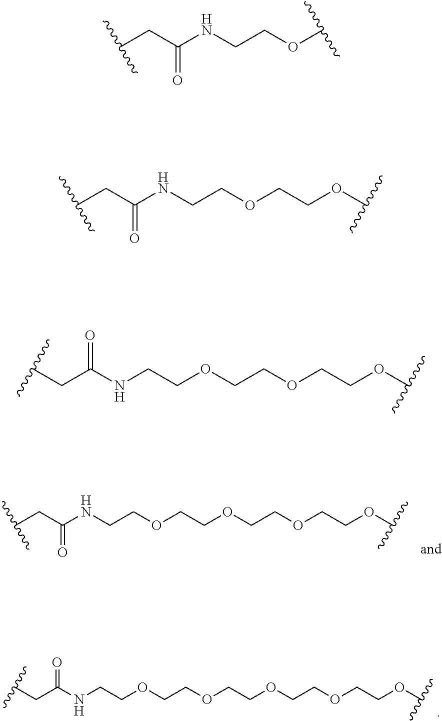 [SMILES: *CC(=O)NCCO* ; *CC(=O)NCCOCCO* ; *CC(=O)NCCOCCOCCO* ; *CC(=O)NCCOCCOCCOCCO* ; *CC(=O)NCCOCCOCCOCCOCCO*]

In certain embodiments, Linker-B is selected from:

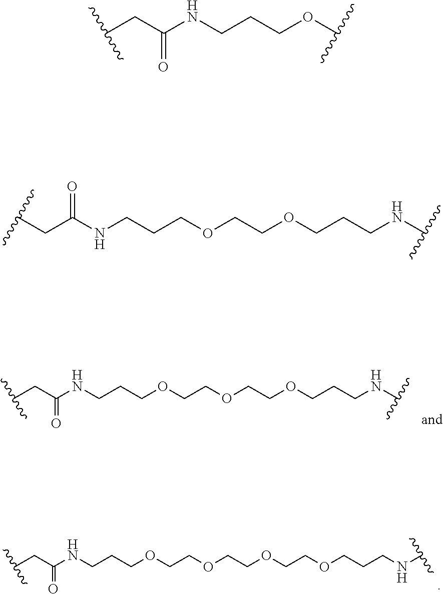 [SMILES: *CC(=O)NCCCO* ; *CC(=O)NCCCOCCOCCCN* ; *CC(=O)NCCCOCCOCCOCCCN* ; *CC(=O)NCCCOCCOCCOCCOCCCN*]

In certain embodiments, Linker-B is selected from:

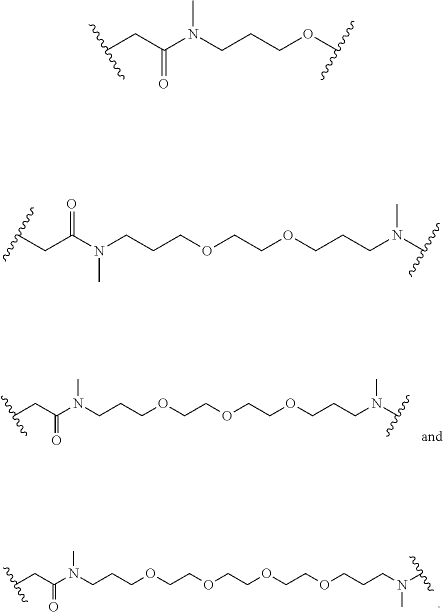 [SMILES: *CC(=O)N(C)CCCO* ; *CC(=O)N(C)CCCOCCOCCCN(*)C ; *CC(=O)N(C)CCCOCCOCCOCCCN(*)C ; *CC(=O)N(C)CCCOCCOCCOCCOCCCN(*)C]

In certain embodiments, Linker-B is selected from:

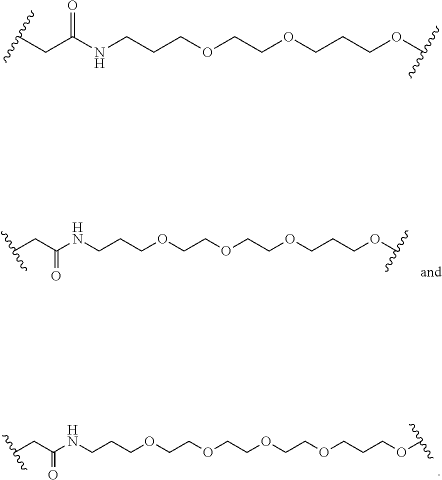 [SMILES: *CC(=O)NCCCOCCOCCCO* ; *CC(=O)NCCCOCCOCCOCCCO* ; *CC(=O)NCCCOCCOCCOCCOCCCO*]

In certain embodiments, Linker-B is selected from:

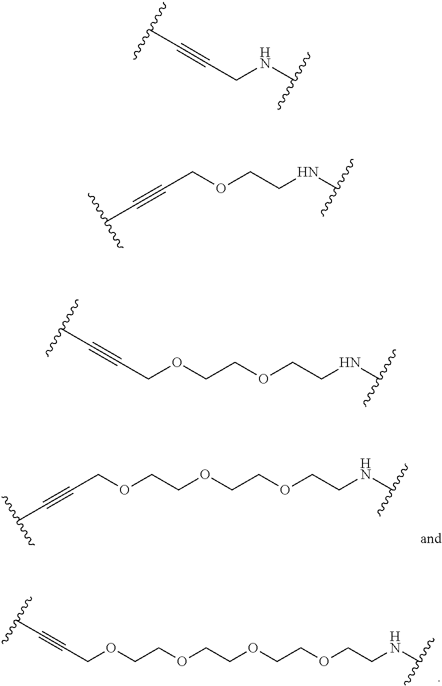 [SMILES: *C#CCN* ; *C#CCOCCN* ; *C#CCOCCOCCN* ; *C#CCOCCOCCOCCN* ; *C#CCOCCOCCOCCOCCN*]

In certain embodiments, Linker-B is selected from:

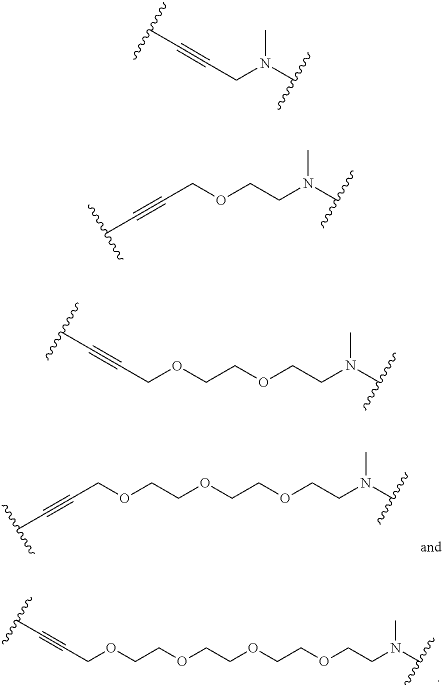

In certain embodiments, Linker-B is selected from:

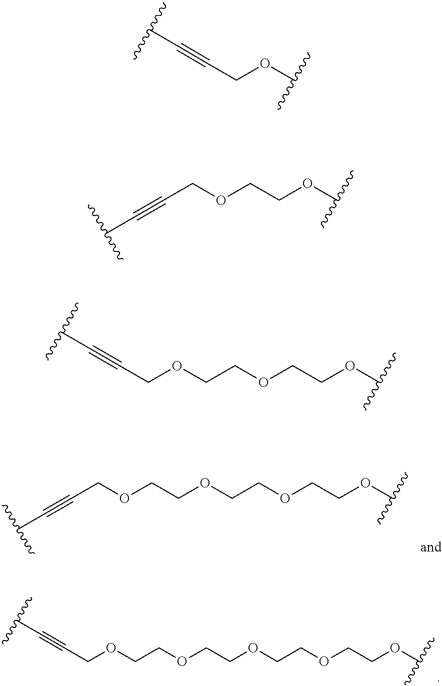 [SMILES: *C#CCO* ; *C#CCOCCO* ; *C#CCOCCOCCO* ; *C#CCOCCOCCOCCO* ; *C#CCOCCOCCOCCOCCO*]

In certain embodiments Linker-A and/or Linker-B is selected from:

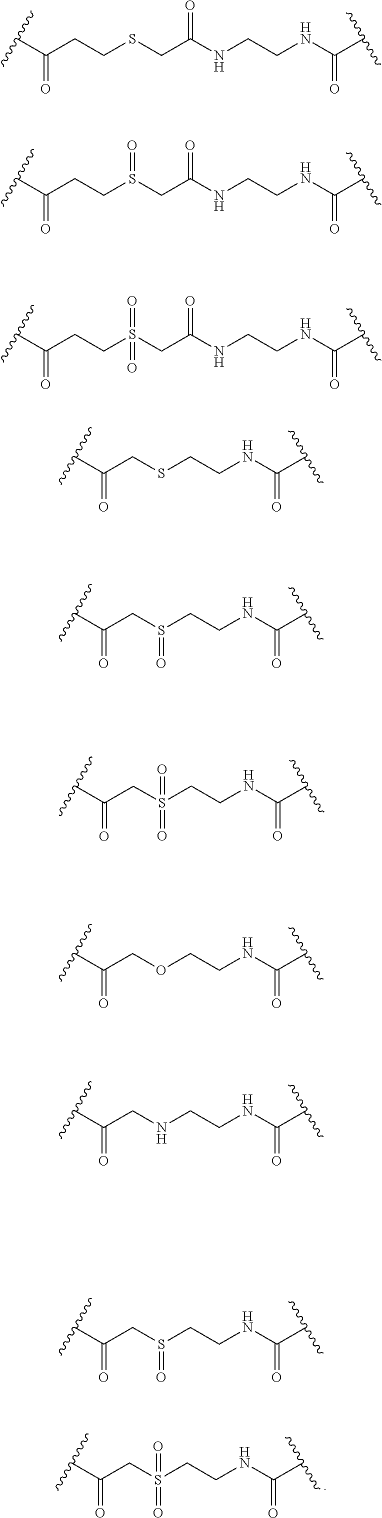

In certain embodiments Linker-A and/or Linker-B is selected from:

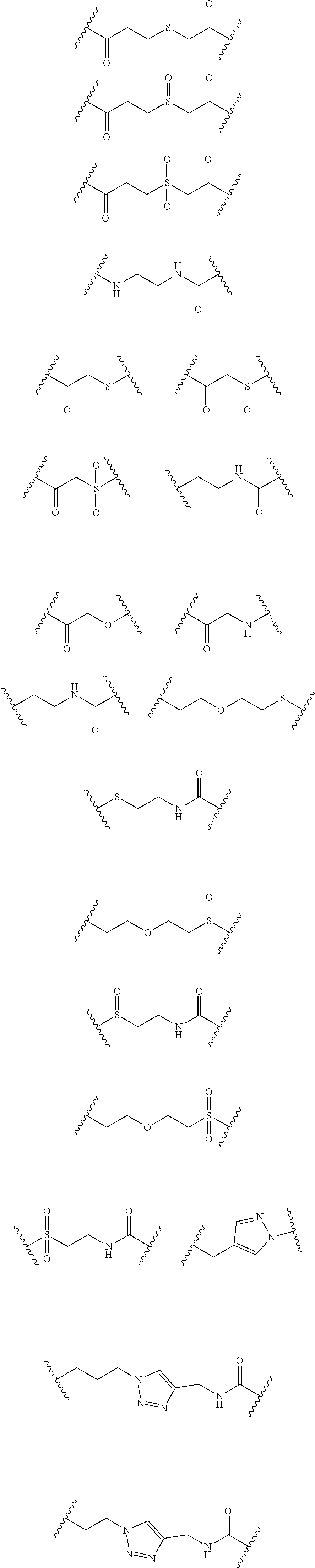

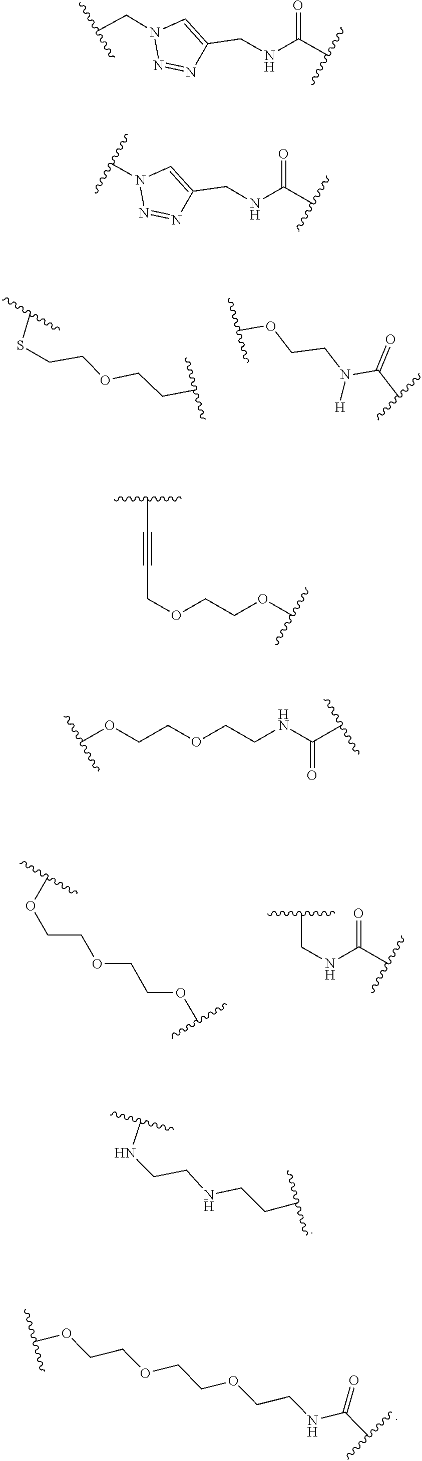

### Usp28 Targeting Ligands

In certain embodiments, the crystal structure of USP28 is searchable by 6HEJ, 2MUU, 6H4I, 6HEK, 6HEI, 2LVA, 6H4H, 6HEH, and 6H4I.

Non-limiting examples of ligands that bind USP28 include those described in Ruiz, E. J. et al. “USP28 deletion and small molecule inhibition destabilises c-Myc and elicits regression of squamous cell lung carcinoma” bioRxiv, 2021, doi:10.1101/2020.11.17.377705; Wrigley, J. D. et al. “Identification and Characterization of Dual Inhibitors of the USP25/28 Deubiquitinating Enzyme Subfamily” ACS Chem. Biol. 2017, 12, 3113-3125; Liu, Z. et al. “Discovery of [1,2,3]triazolo[4,5-d]pyrimiding derivatives as highly potent, selective, and cellularly active USP28 inhibitors” Acta Pharm. Sin. B 2020, 10(8), 1476-1491; Guerin, D. J. et al. US2019/0359628, U.S. Pat. No. 10,913,753, WO 2020/033709, WO 2017/139779; Peng, J. et al. WO 2020/224652; CN 112898314; and CN 111909181.

In certain embodiments the USP28 Targeting Ligand of the present invention is selected from:

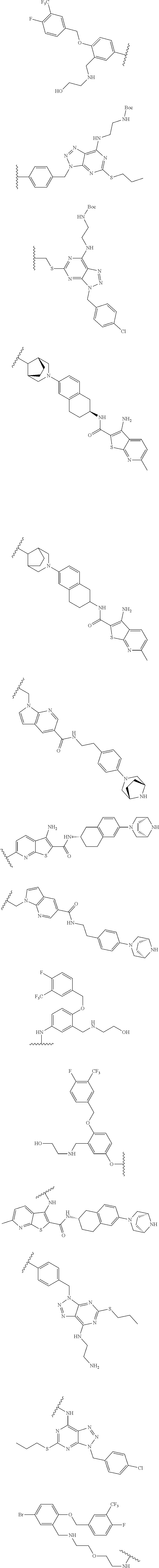

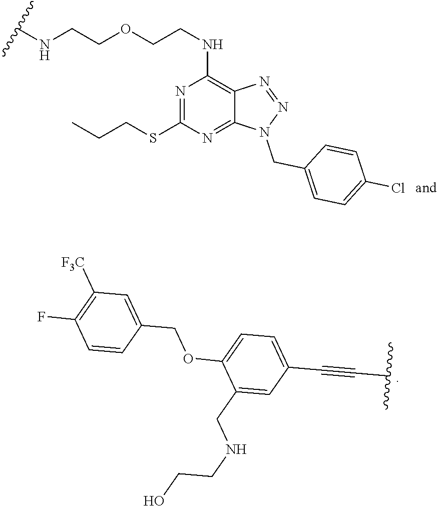 [SMILES: *NCCOCCNc1nc(SCCC)nc2c1nnn2Cc1ccc(Cl)cc1 ; *C#Cc1ccc(OCc2ccc(F)c(C(F)(F)F)c2)c(CNCCO)c1]

In certain embodiments the USP28 Targeting Ligand of the present invention is selected from:

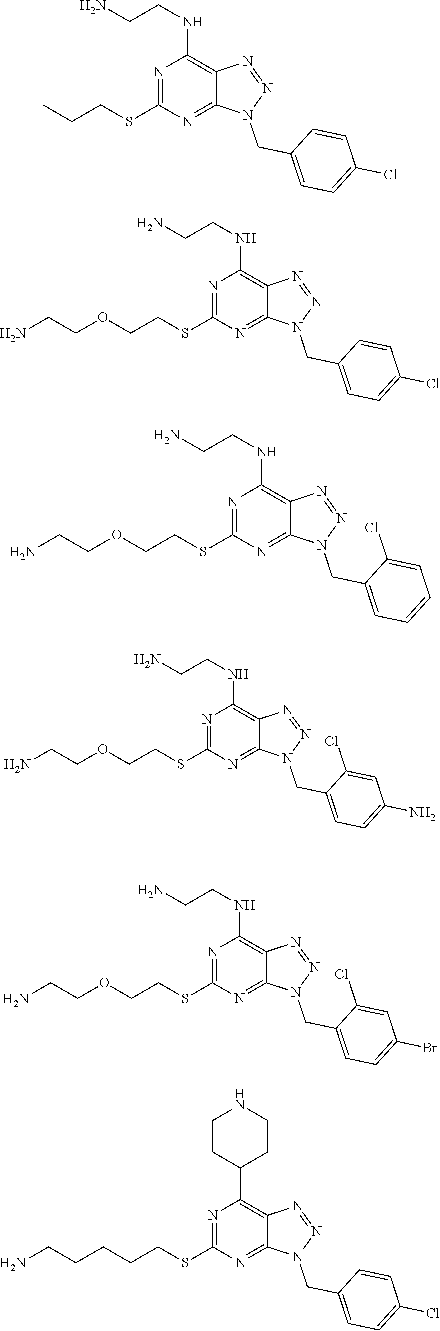

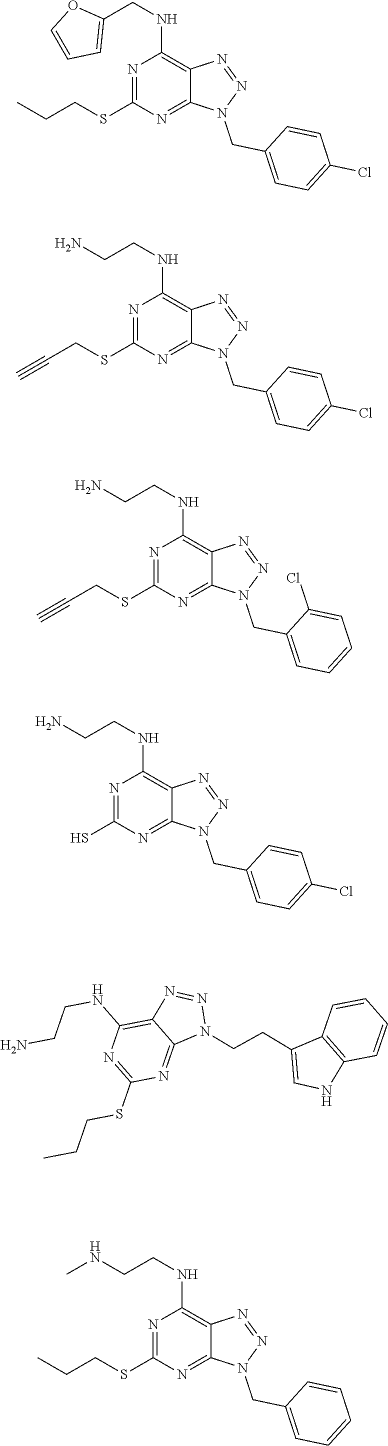

wherein the attachment point to the Linker-Ubiquitinated Protein Targeting Ligand is made at an atom allowed by valence or replaces a substituent for example non-limiting examples of attachment points for

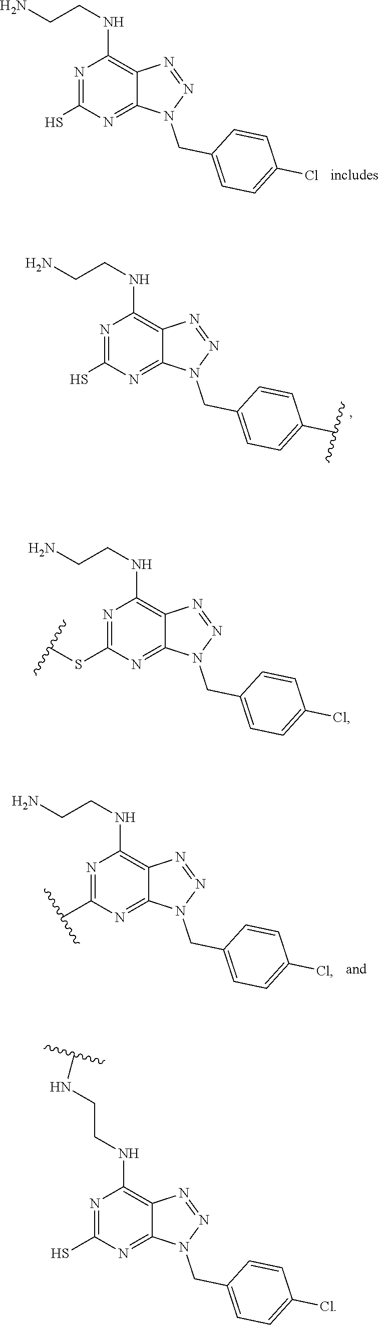 [SMILES: NCCNc1nc(S)nc2c1nnn2Cc1ccc(Cl)cc1 ; *c1ccc(Cn2nnc3c(NCCN)nc(S)nc32)cc1 ; *Sc1nc(NCCN)c2nnn(Cc3ccc(Cl)cc3)c2n1 ; *c1nc(NCCN)c2nnn(Cc3ccc(Cl)cc3)c2n1 ; *NCCNc1nc(S)nc2c1nnn2Cc1ccc(Cl)cc1]

In certain embodiments the USP28 Targeting Ligand of the present invention is selected from:

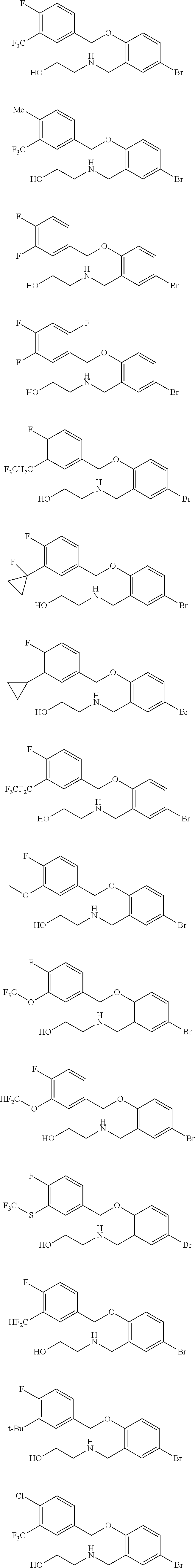

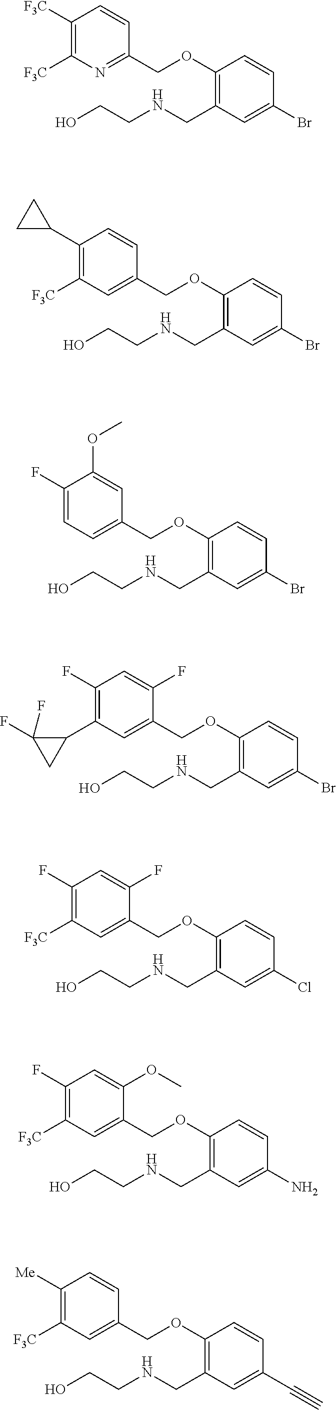

wherein the attachment point to the Linker-Ubiquitinated Protein Targeting Ligand is made at an atom allowed by valence or replaces a substituent.

In certain embodiments the USP28 Targeting Ligand of the present invention is selected from:

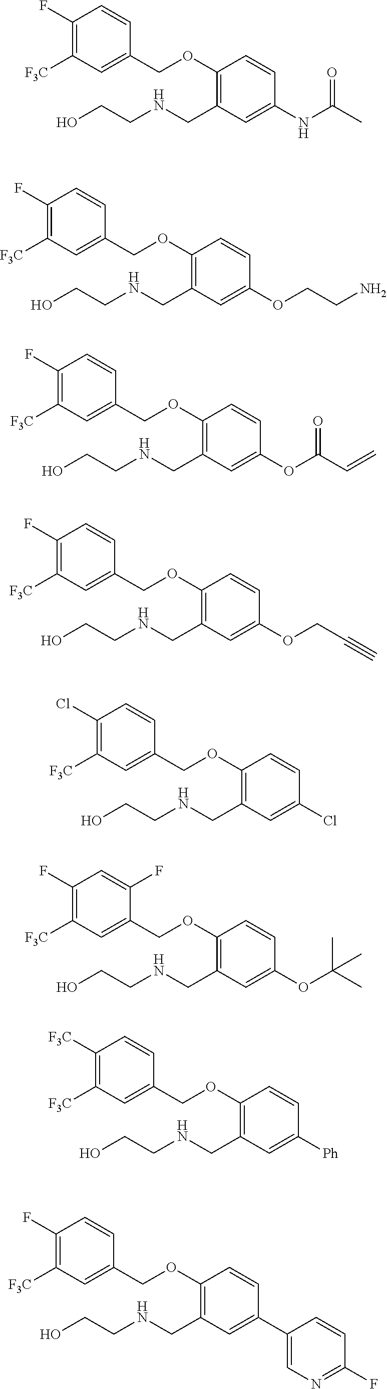

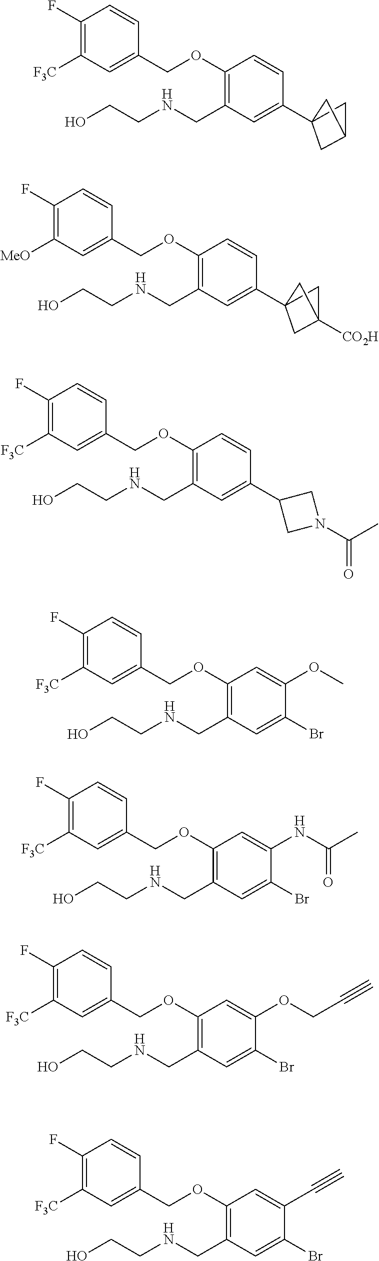

wherein the attachment point to the Linker-Ubiquitinated Protein Targeting Ligand is made at an atom allowed by valence or replaces a substituent.

In certain embodiments the USP28 Targeting Ligand of the present invention is selected from:

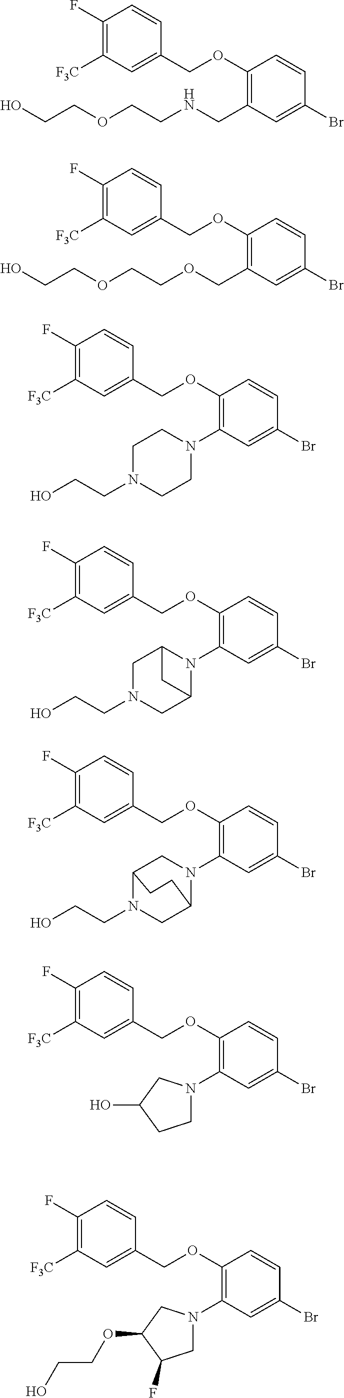

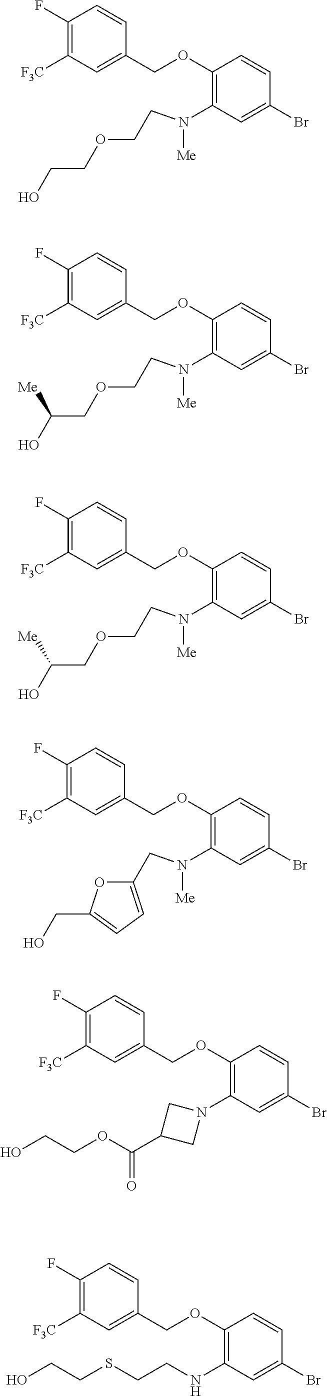

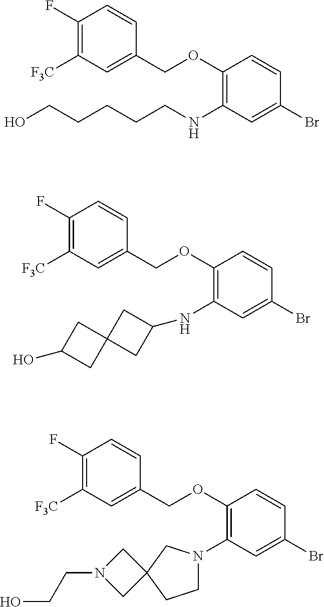

wherein the attachment point to the Linker-Ubiquitinated Protein Targeting Ligand is made at an atom allowed by valence or replaces a substituent.

In certain embodiments the USP28 Targeting Ligand of the present invention is selected from:

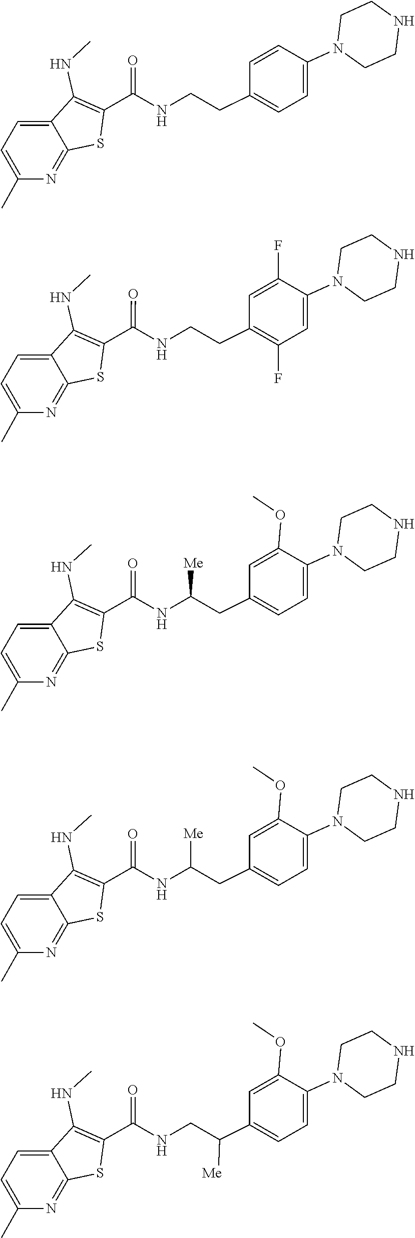

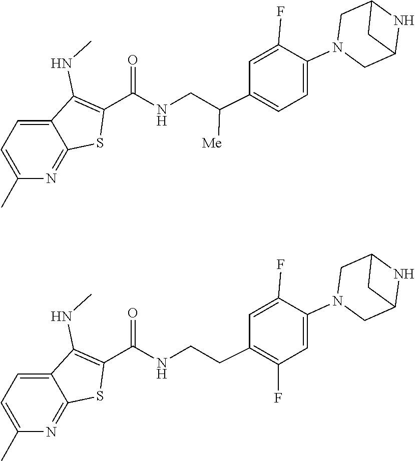

wherein the attachment point to the Linker-Ubiquitinated Protein Targeting Ligand is made at an atom allowed by valence or replaces a substituent.

In certain embodiments the USP28 Targeting Ligand of the present invention is selected from:

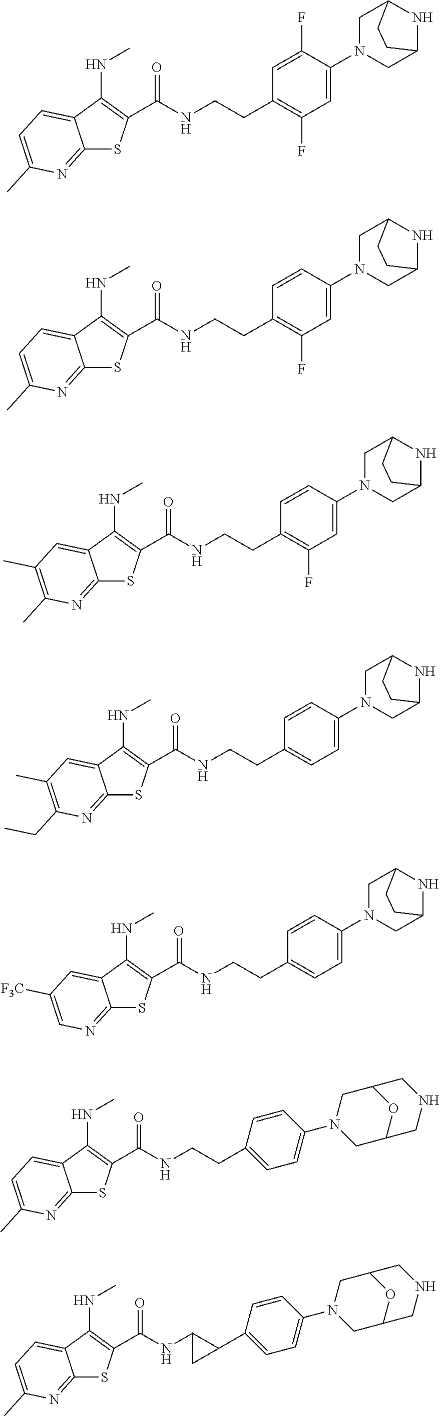

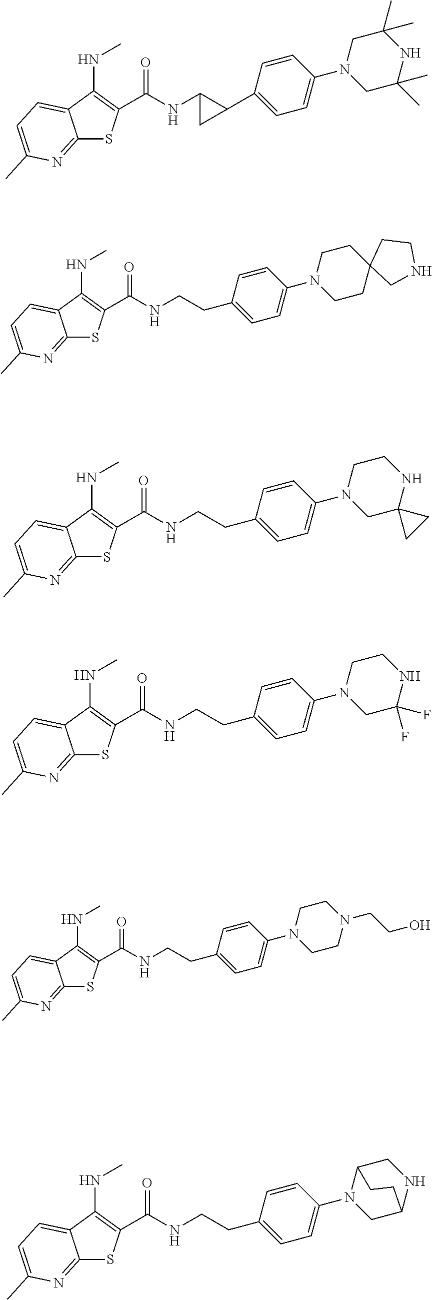

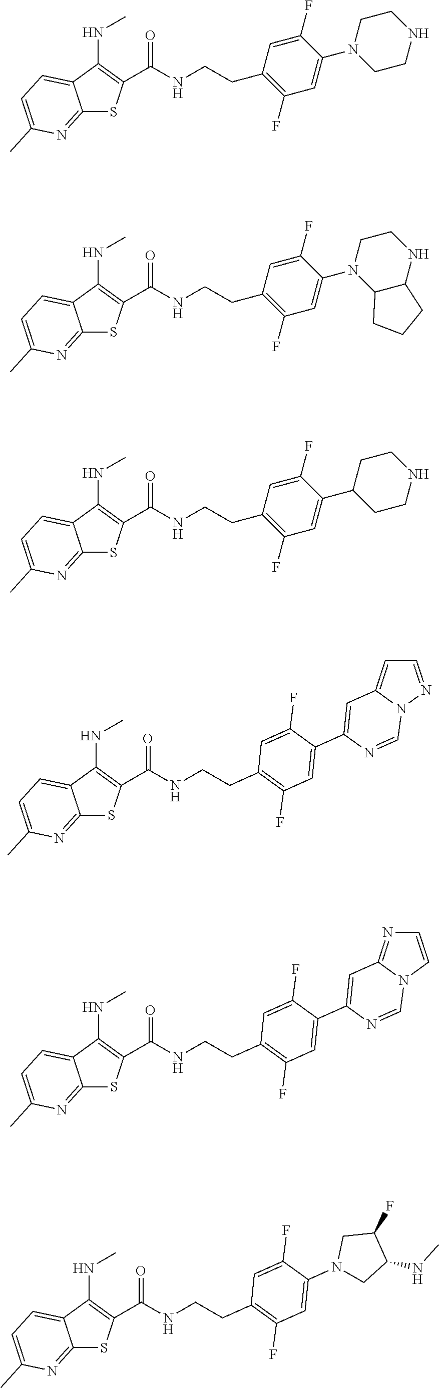

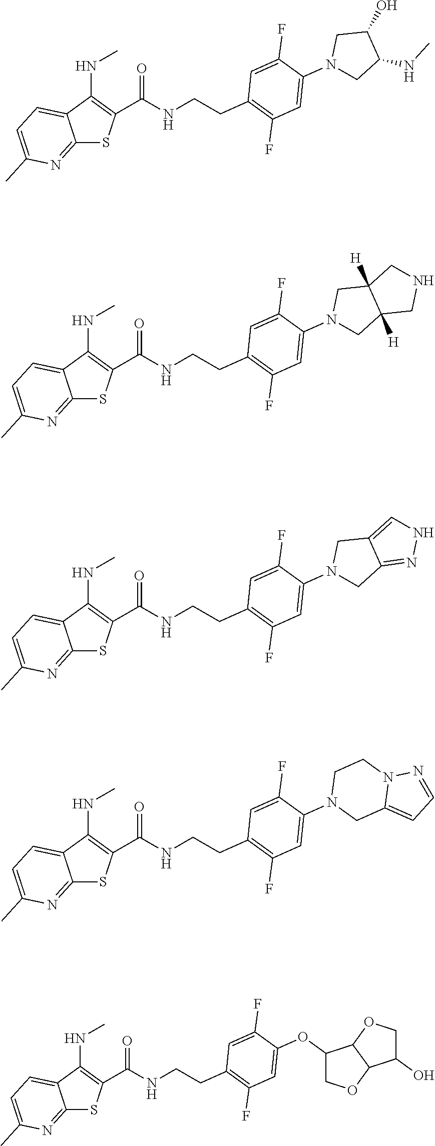

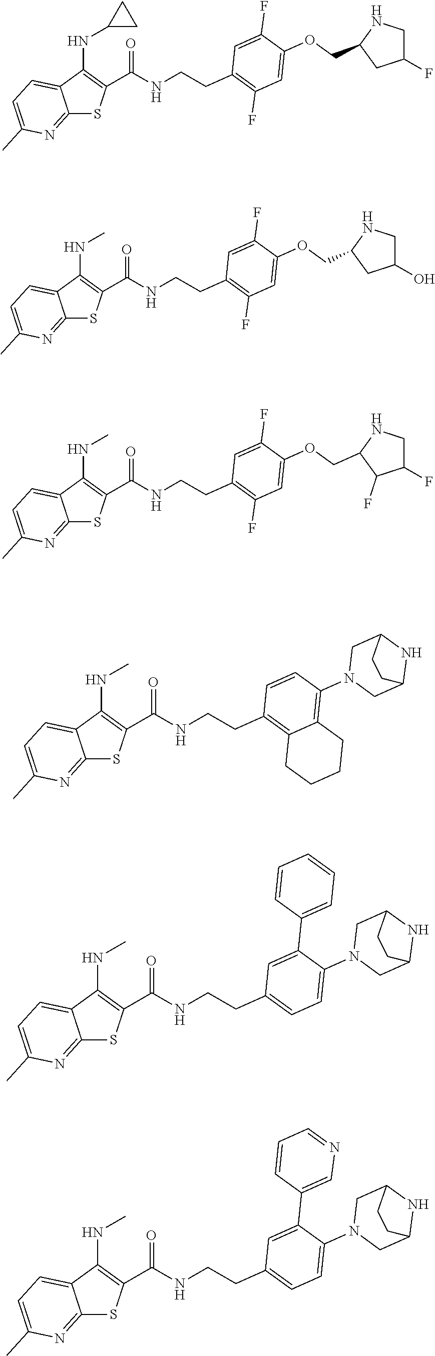

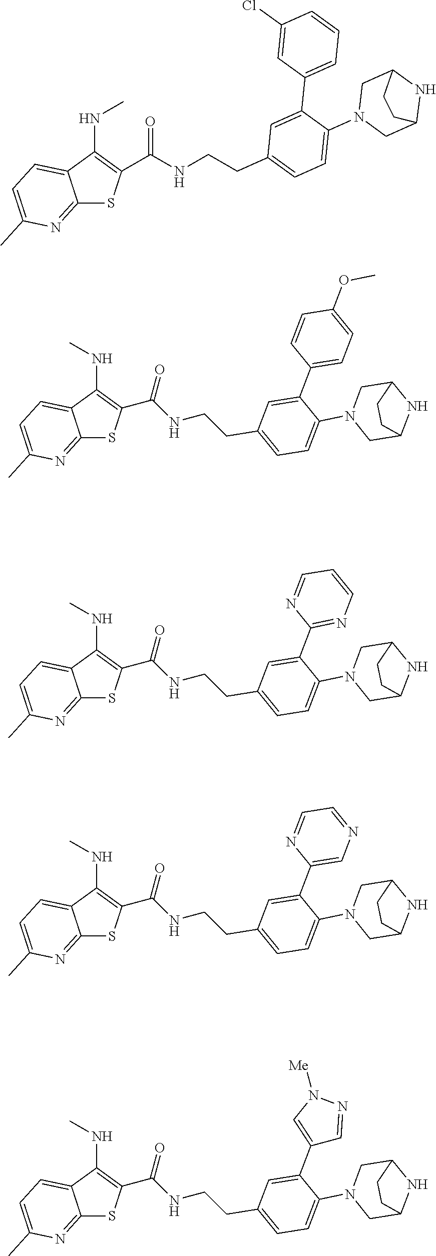

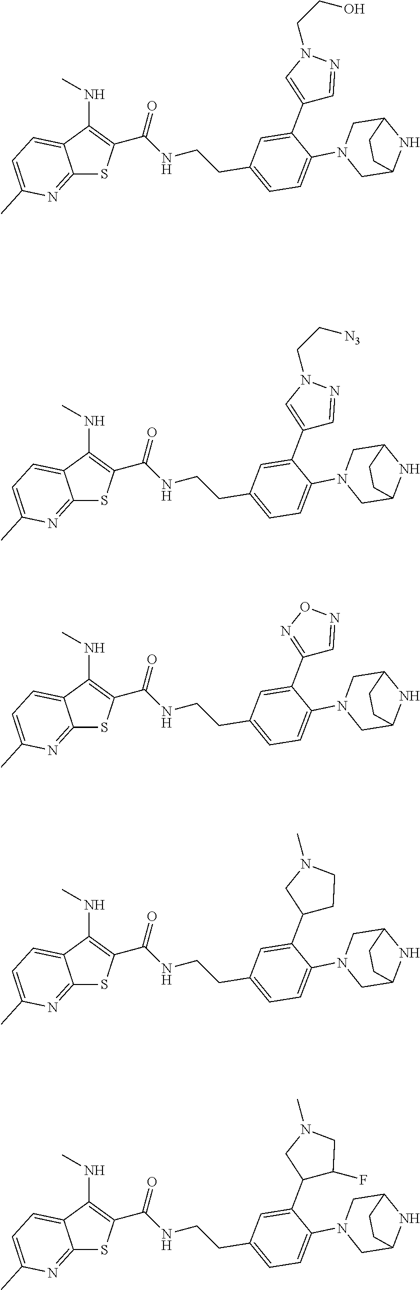

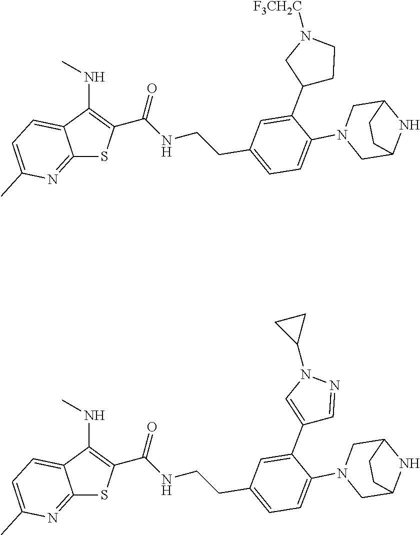

wherein the attachment point to the Linker-Ubiquitinated Protein Targeting Ligand is made at an atom allowed by valence or replaces a substituent.

In certain embodiments the USP28 Targeting Ligand of the present invention is selected from:

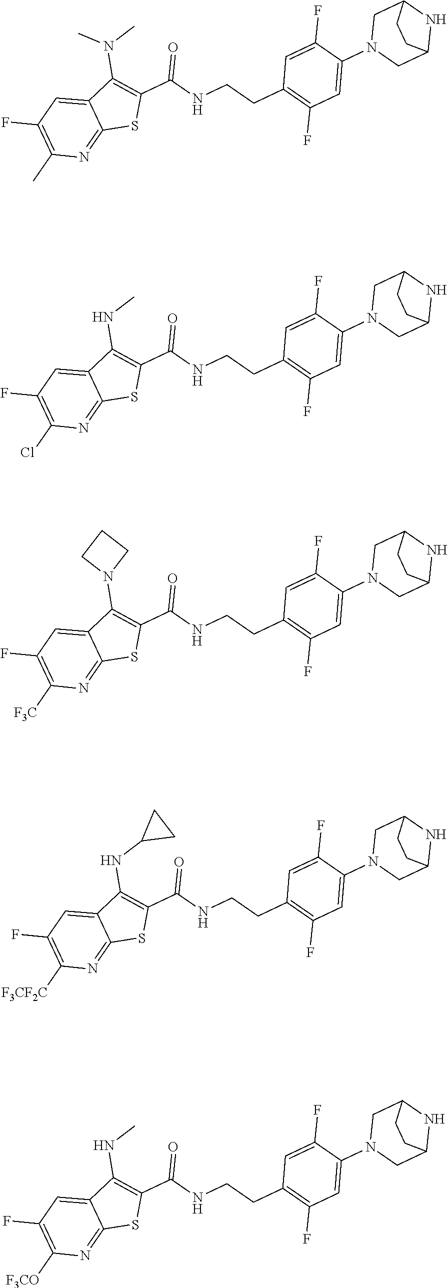

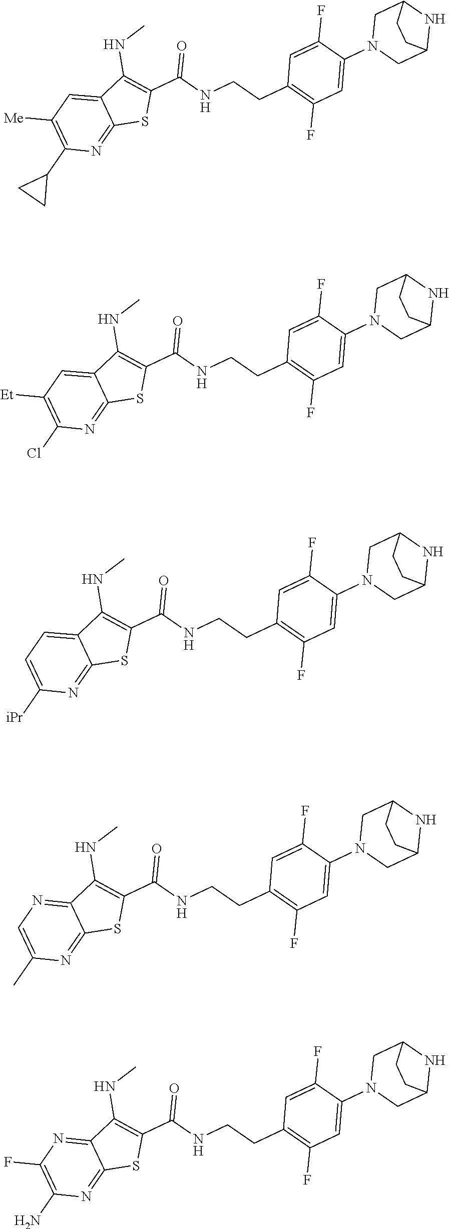

wherein the attachment point to the Linker-Ubiquitinated Protein Targeting Ligand is made at an atom allowed by valence or replaces a substituent.

In certain embodiments the USP28 Targeting Ligand of the present invention is selected from:

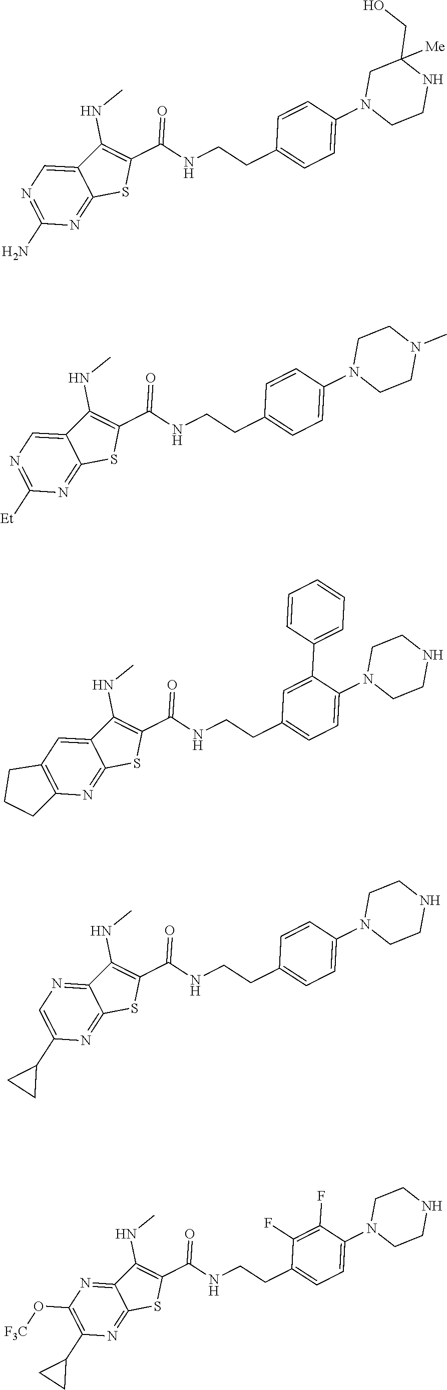

wherein the attachment point to the Linker-Ubiquitinated Protein Targeting Ligand is made at an atom allowed by valence or replaces a substituent.

In certain embodiments the USP28 Targeting Ligand of the present invention is selected from:

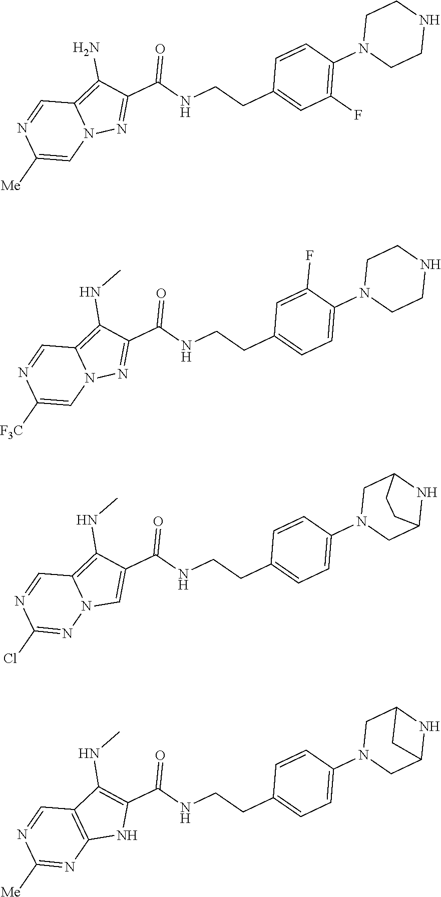

wherein the attachment point to the Linker-Ubiquitinated Protein Targeting Ligand is made at an atom allowed by valence or replaces a substituent.

In certain embodiments the USP28 Targeting Ligand of the present invention is selected from:

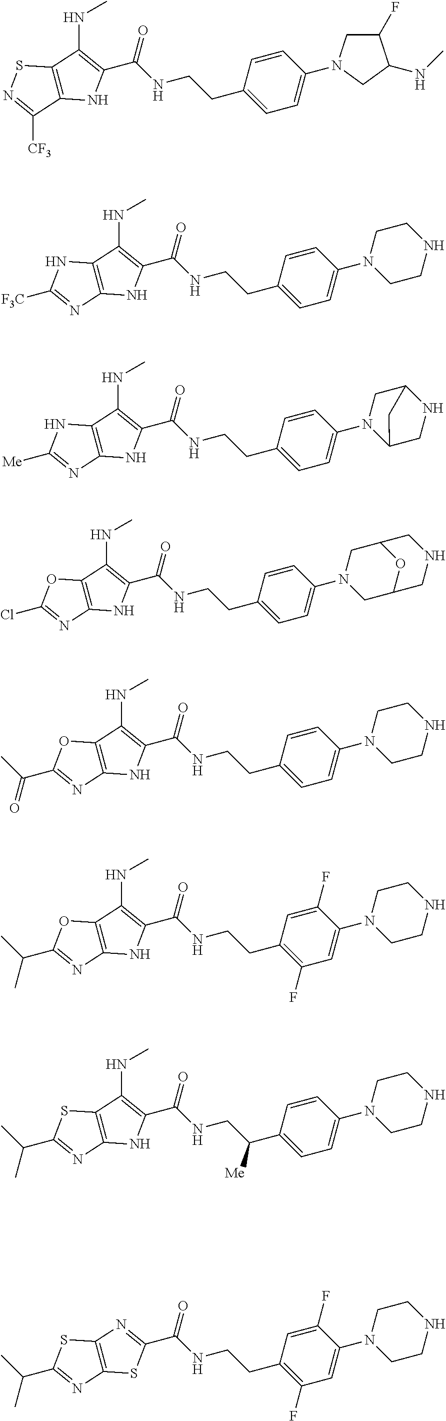

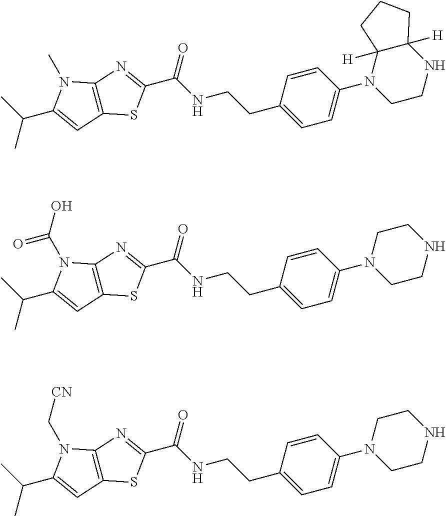

wherein the attachment point to the Linker-Ubiquitinated Protein Targeting Ligand is made at an atom allowed by valence or replaces a substituent.

In certain embodiments the USP28 Targeting Ligand of the present invention is selected from:

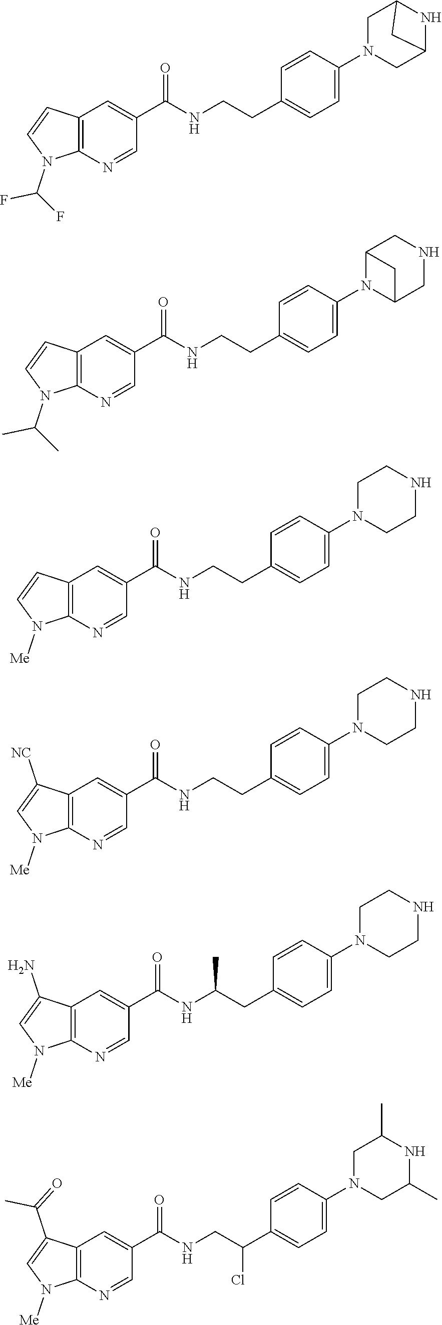

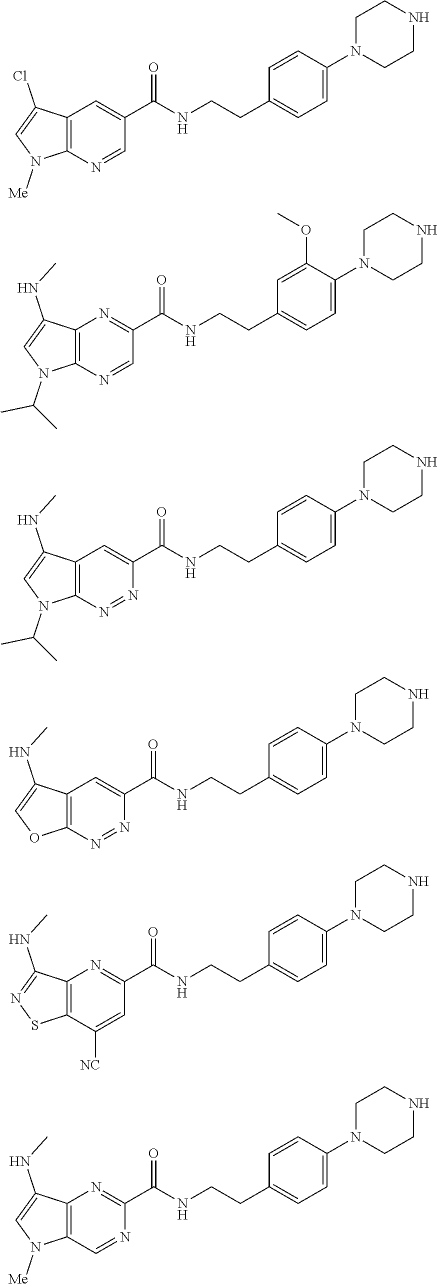

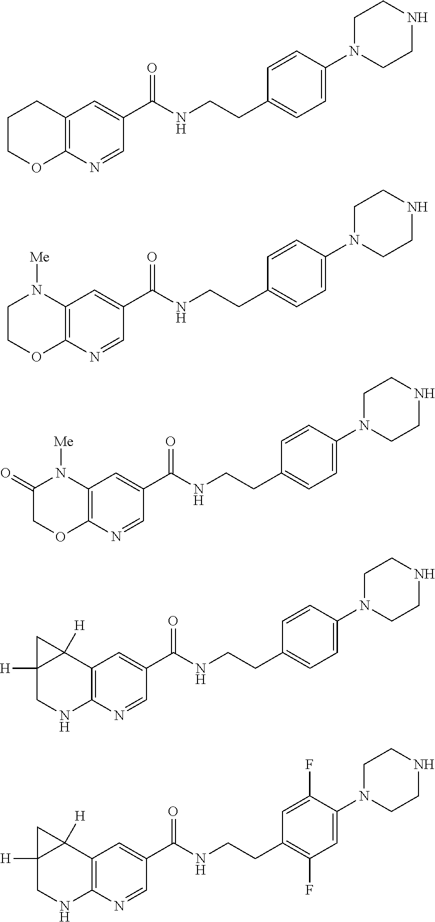

wherein the attachment point to the Linker-Ubiquitinated Protein Targeting Ligand is made at an atom allowed by valence or replaces a substituent.

In certain embodiments the USP28 Targeting Ligand of the present invention is selected from:

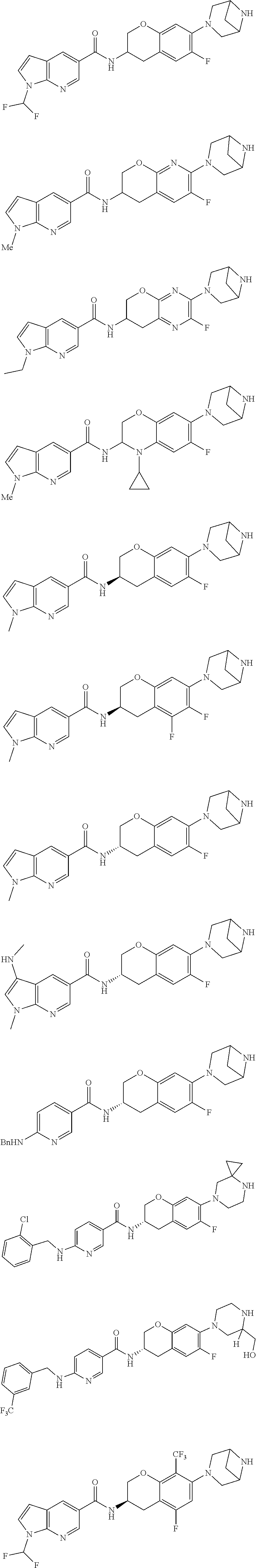

wherein the attachment point to the Linker-Ubiquitinated Protein Targeting Ligand is made at an atom allowed by valence or replaces a substituent.

In certain embodiments the USP28 Targeting Ligand of the present invention is selected from:

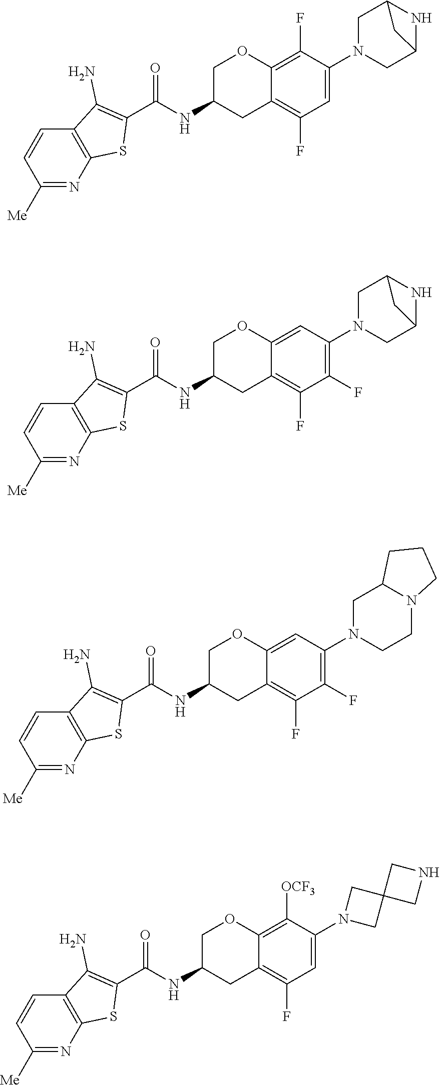

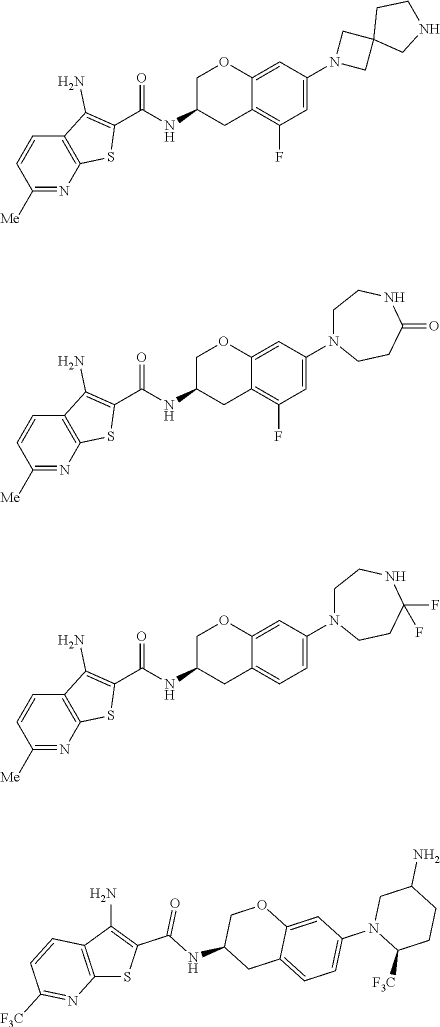

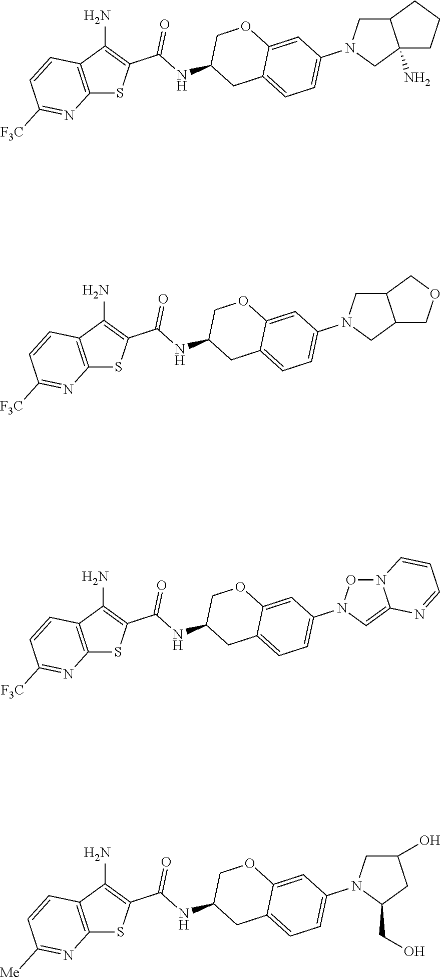

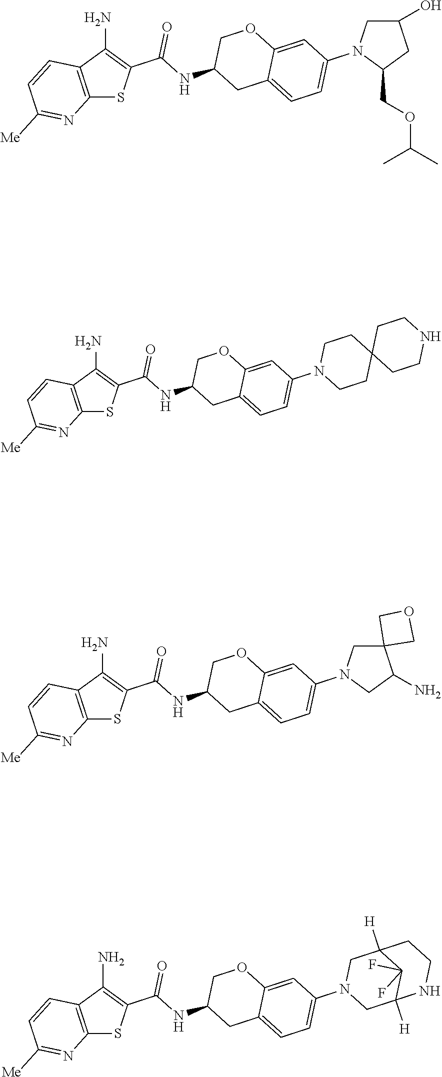

wherein the attachment point to the Linker-Ubiquitinated Protein Targeting Ligand is made at an atom allowed by valence or replaces a substituent.

In certain embodiments the USP28 Targeting Ligand is selected from:

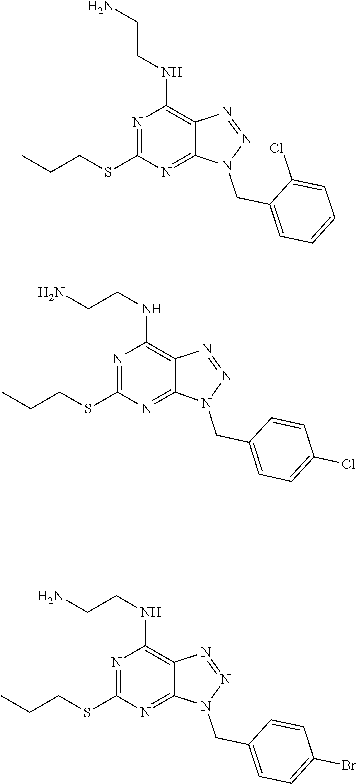 [SMILES: CCCSc1nc(NCCN)c2nnn(Cc3ccccc3Cl)c2n1 ; CCCSc1nc(NCCN)c2nnn(Cc3ccc(Cl)cc3)c2n1 ; CCCSc1nc(NCCN)c2nnn(Cc3ccc(Br)cc3)c2n1]

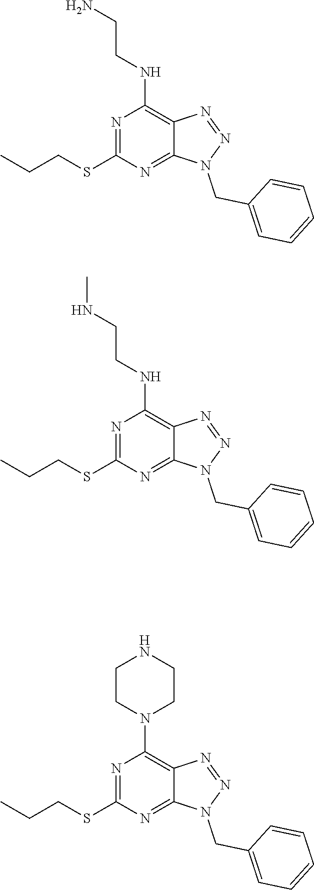 [SMILES: CCCSc1nc(NCCN)c2nnn(Cc3ccccc3)c2n1 ; CCCSc1nc(NCCNC)c2nnn(Cc3ccccc3)c2n1 ; CCCSc1nc(N2CCNCC2)c2nnn(Cc3ccccc3)c2n1]

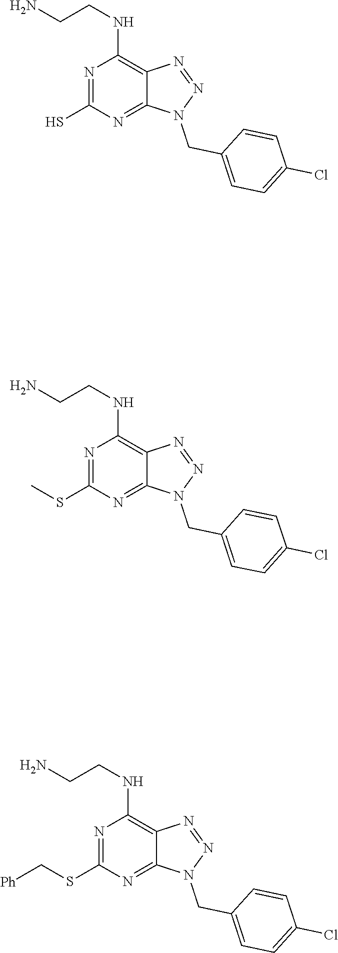 [SMILES: NCCNc1nc(S)nc2c1nnn2Cc1ccc(Cl)cc1 ; CSc1nc(NCCN)c2nnn(Cc3ccc(Cl)cc3)c2n1 ; NCCNc1nc(SCc2ccccc2)nc2c1nnn2Cc1ccc(Cl)cc1]

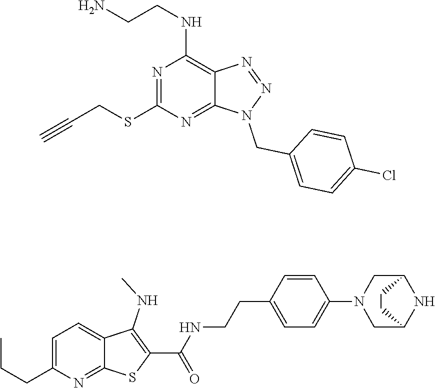 [SMILES: C#CCSc1nc(NCCN)c2nnn(Cc3ccc(Cl)cc3)c2n1 ; CCCc1ccc2c(NC)c(C(=O)NCCc3ccc(N4C[C@H]5CC[C@@H](C4)N5)cc3)sc2n1]

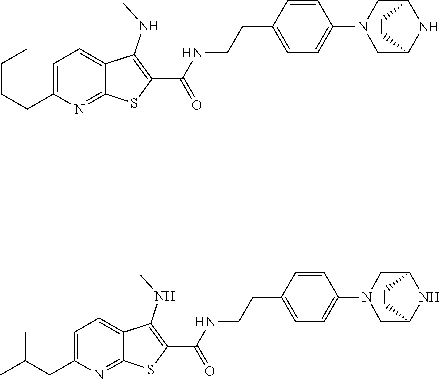 [SMILES: CCCCc1ccc2c(NC)c(C(=O)NCCc3ccc(N4C[C@H]5CC[C@@H](C4)N5)cc3)sc2n1 ; CNc1c(C(=O)NCCc2ccc(N3C[C@H]4CC[C@@H](C3)N4)cc2)sc2nc(CC(C)C)ccc12]

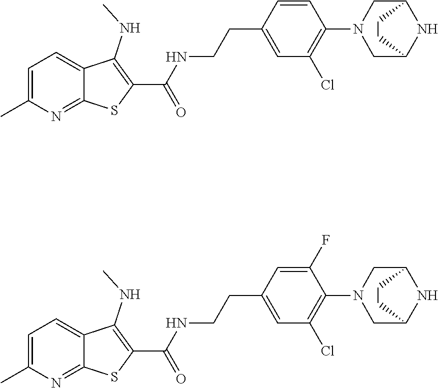 [SMILES: CNc1c(C(=O)NCCc2ccc(N3C[C@H]4CC[C@@H](C3)N4)c(Cl)c2)sc2nc(C)ccc12 ; CNc1c(C(=O)NCCc2cc(F)c(N3C[C@H]4CC[C@@H](C3)N4)c(Cl)c2)sc2nc(C)ccc12]

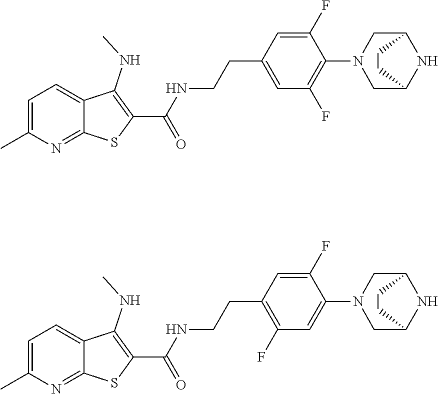 [SMILES: CNc1c(C(=O)NCCc2cc(F)c(N3C[C@H]4CC[C@@H](C3)N4)c(F)c2)sc2nc(C)ccc12 ; CNc1c(C(=O)NCCc2cc(F)c(N3C[C@H]4CC[C@@H](C3)N4)cc2F)sc2nc(C)ccc12]

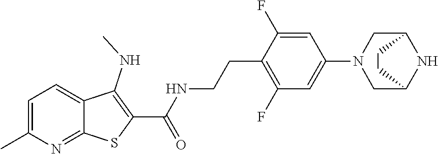 [SMILES: CNc1c(C(=O)NCCc2c(F)cc(N3C[C@H]4CC[C@@H](C3)N4)cc2F)sc2nc(C)ccc12]

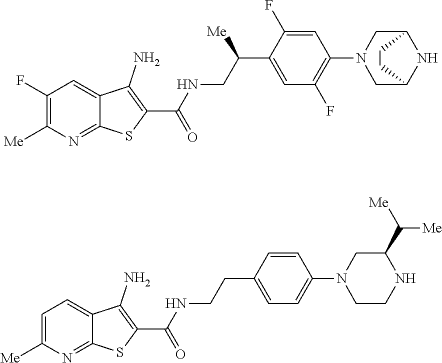

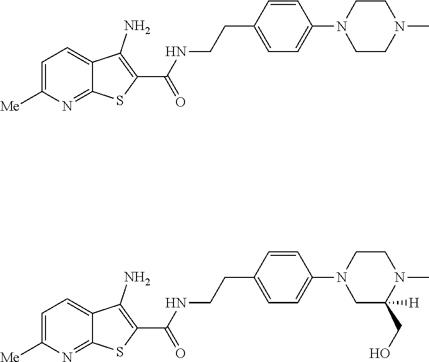 [SMILES: Cc1ccc2c(N)c(C(=O)NCCc3ccc(N4CCN(C)CC4)cc3)sc2n1 ; Cc1ccc2c(N)c(C(=O)NCCc3ccc(N4CCN(C)[C@@H](CO)C4)cc3)sc2n1]

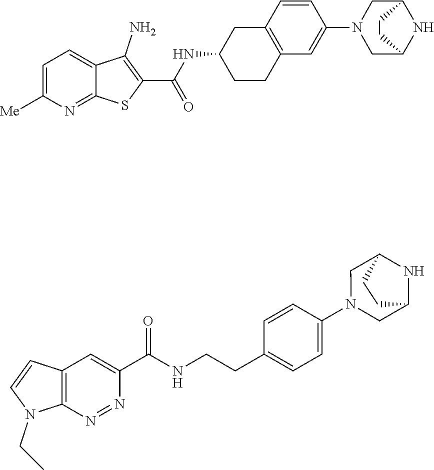 [SMILES: Cc1ccc2c(N)c(C(=O)N[C@H]3CCc4cc(N5C[C@H]6CC[C@@H](C5)N6)ccc4C3)sc2n1 ; CCn1ccc2cc(C(=O)NCCc3ccc(N4C[C@H]5CC[C@@H](C4)N5)cc3)nnc21]

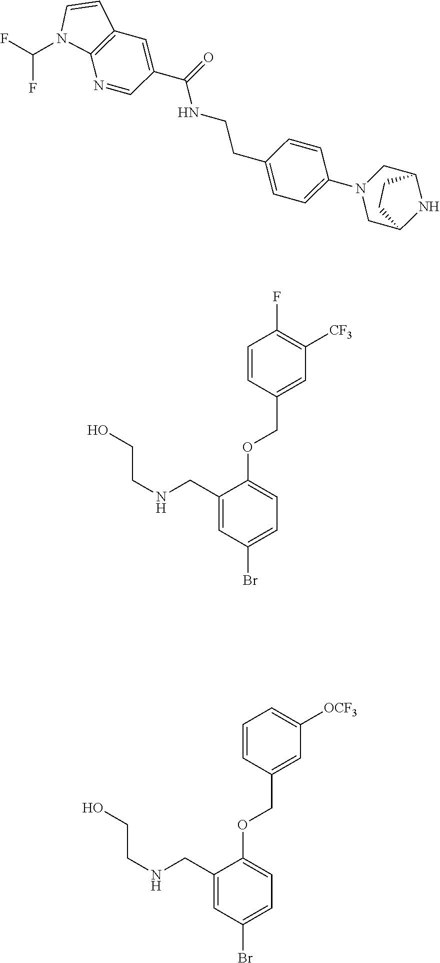 [SMILES: O=C(NCCc1ccc(N2C[C@H]3CC[C@@H](C2)N3)cc1)c1cnc2c(ccn2C(F)F)c1 ; OCCNCc1cc(Br)ccc1OCc1ccc(F)c(C(F)(F)F)c1 ; OCCNCc1cc(Br)ccc1OCc1cccc(OC(F)(F)F)c1]

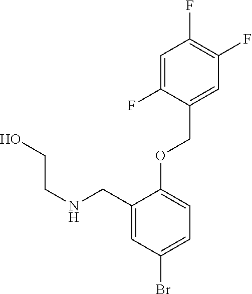 [SMILES: OCCNCc1cc(Br)ccc1OCc1cc(F)c(F)cc1F]

or a pharmaceutically acceptable salt thereof, wherein each of the above USP28 Targeting Ligands is substituted by 1-Linker-Ubiquitinated Protein Target Ligand and 0, 1, 2, or 3, R¹⁰¹ substituents; and

R¹⁰¹ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²¹

### Specific Target Protein Stabilizing Compounds of the Present Invention

1. A compound of Formula

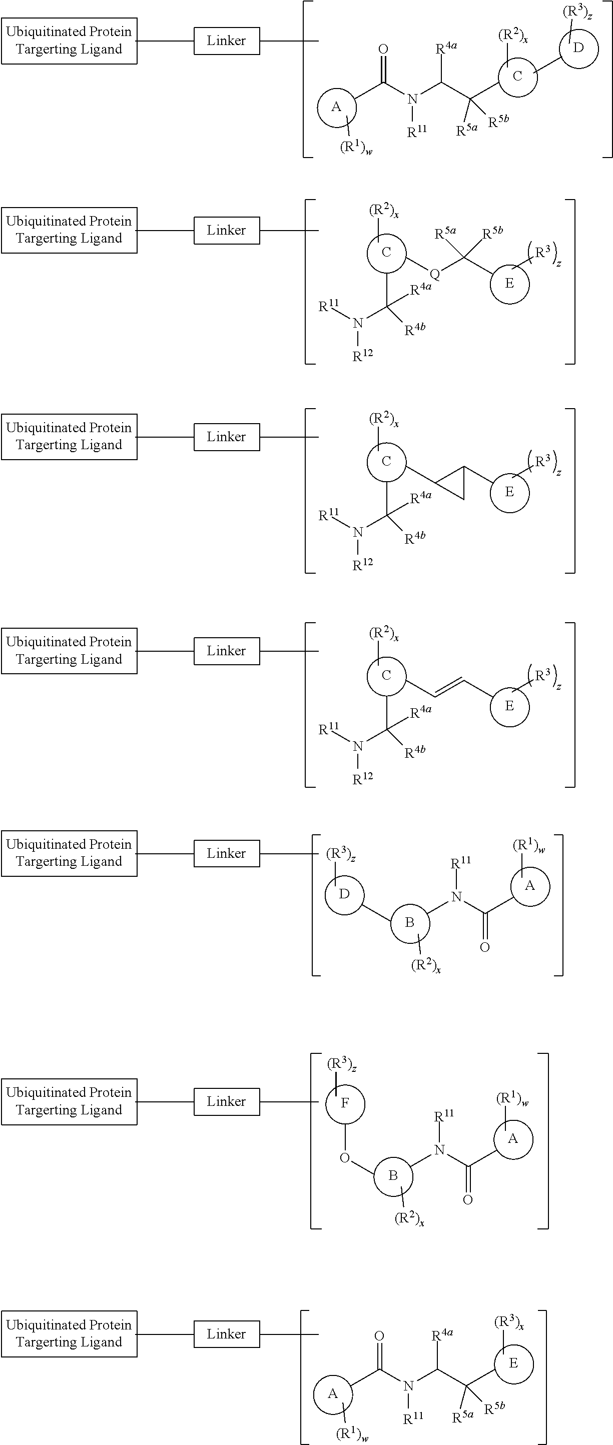

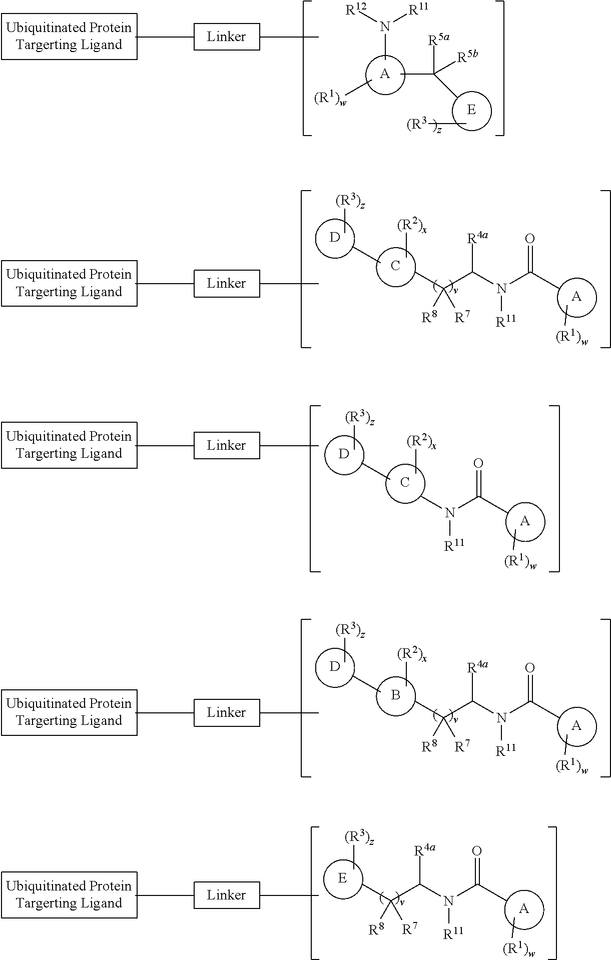

or a pharmaceutically acceptable salt thereof.
wherein:
v is 0, 1, 2, or 3;
w is 0, 1, 2, 3, or 4 as allowed by valence;
x is 0, 1, 2, 3, or 4 as allowed by valence;
z is 0, 1, 2, 3, or 4 as allowed by valence;
Q is 0, NR¹¹, CR⁷R⁸, or S;
R¹ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²¹;
R² is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²²;
R³ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²³;
R^{4a} and R^{5a} are independently selected at each instance from hydrogen, halogen, alkyl, haloalkyl, alkenyl, alkynyl, heterocycle, aryl, and heteroaryl, wherein each alkyl, haloalkyl, alkenyl, alkynyl, heterocycle, aryl, and heteroaryl is optionally substituted as allowed by valence with 1, 2, 3, or 4 substituents selected from R²⁴;
R^{4b} and R^{5b} are independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²⁵;
or R^{4a} and R^{4b} together with the atom to which they are attached are combined to form a spirocycle;
or R^{5a} and R^{5b} together with the atom to which they are attached are combined to form a spirocycle;
R⁶ is hydrogen, cyano, alkyl, haloalkyl, alkenyl, alkynyl, aryl, heterocycle, heteroaryl, —C(O)R⁴⁰, —S(O)R⁴⁰, and —S(O)₂R⁴⁰; each of which alkyl, haloalkyl, alkenyl, alkynyl, aryl, heterocycle, and heteroaryl is optionally substituted as allowed by valence with 1, 2, 3, or 4 substituents selected from R³¹;
each R⁷ and R⁸ is independently selected from hydrogen, alkyl, and haloalkyl;
in certain embodiments R⁷ and R⁸ are both hydrogen;
R¹⁰ is independently selected at each instance from hydrogen, alkyl, haloalkyl, alkenyl, alkynyl, —OR¹¹, —NR¹¹R¹², —SR¹¹, aryl, heterocycle, and heteroaryl; each of which alkyl, haloalkyl, alkenyl, alkynyl, heterocycle, aryl, and heteroaryl is optionally substituted as allowed by valence with 1, 2, 3, or 4 substituents selected from R³⁰;
R¹¹ and R¹² are independently selected at each instance from hydrogen, alkyl, haloalkyl, alkenyl, alkynyl, aryl, heterocycle, heteroaryl, —C(O)R⁴⁰, —S(O)R⁴⁰, and —S(O)₂R⁴⁰; each of which alkyl, haloalkyl, alkenyl, alkynyl, aryl, heterocycle, and heteroaryl is optionally substituted as allowed by valence with 1, 2, 3, or 4 substituents selected from R³¹;
R²¹, R²², R²³, R²⁴, R²⁵, and R²⁶ are independently selected at each instance from hydrogen, halogen, alkyl, haloalkyl, alkenyl, alkynyl, heterocycle, aryl, heteroaryl, cyano, nitro, —C(O)R⁴⁰, —OC(O)R⁴⁰, —NR⁴¹C(O)R⁴⁰, —OR⁴¹, —NR⁴¹R⁴², —S(O)R⁴⁰, —S(O)₂R⁴⁰, —OS(O)R⁴⁰, —OS(O)₂R⁴⁰, —NR⁴¹S(O)R⁴⁰, —NR⁴¹S(O)₂R⁴⁰, and —SR⁴¹, wherein each alkyl, haloalkyl, alkenyl, alkynyl, heterocycle, aryl, and heteroaryl is optionally substituted as allowed by valence with 1, 2, 3, or 4 substituents selected from R⁴³;
R³⁰ and R³¹ are independently selected at each instance from hydrogen, halogen, alkyl, haloalkyl, alkenyl, alkynyl, heterocycle, aryl, heteroaryl, cyano, nitro, —C(O)R⁴⁰, —OC(O)R⁴⁰, —NR⁴¹C(O)R⁴⁰, —OR⁴¹, —NR⁴¹R⁴², —S(O)R⁴⁰, —S(O)₂R⁴⁰, —OS(O)R⁴⁰, —OS(O)₂R⁴⁰, —NR⁴¹S(O)R⁴⁰, —NR⁴¹S(O)₂R⁴⁰, and —SR⁴¹, wherein each alkyl, haloalkyl, alkenyl, alkynyl, heterocycle, aryl, and heteroaryl is optionally substituted as allowed by valence with 1, 2, 3, or 4 substituents selected from R⁴³;
R⁴⁰ is independently selected at each instance from hydrogen, alkyl, haloalkyl, alkenyl, alkynyl, aryl, heterocycle, heteroaryl, amino, hydroxyl, alkoxy, —NHalkyl, and —N(alkyl)₂, each of which except hydrogen is optionally substituted as allowed by valence with 1, 2, 3, or 4 substituents selected from R⁴³;
R⁴¹ and R⁴² are independently selected at each instance from hydrogen, alkyl, haloalkyl, alkenyl, alkynyl, aryl, heterocycle, and heteroaryl; each of which except hydrogen is optionally substituted as allowed by valence with 1, 2, 3, or 4 substituents selected from R⁴³;
R⁴³ is independently selected at each instance from hydrogen, halogen, cyano, nitro, alkyl, haloalkyl, alkenyl, alkynyl, aryl, heterocycle, heteroaryl, amino, hydroxyl, alkoxy, —NHalkyl, —N(alkyl)₂, —OC(O)alkyl, —NHC(O)alkyl, and —N(alkyl)C(O)alkyl;

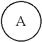

is a aryl, heteroaryl, or bicycle;

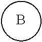

is a bicycle;

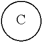

is aryl, heteroaryl, or bicycle;

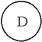

is a heterocycle;

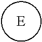

is aryl or heteroaryl; and

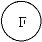

is a heterocycle bonded through a carbon atom;
the Linker is a bond or a bivalent moiety that links the Ubiquitinated Protein Targeting Ligand and the USP28 and/or USP25 Targeting Ligand; and
the Ubiquitinated Protein Targeting Ligand is a ligand that binds a Target Ubiquitinated Protein.

2. The compound of embodiment 1, wherein the compound is of Formula:

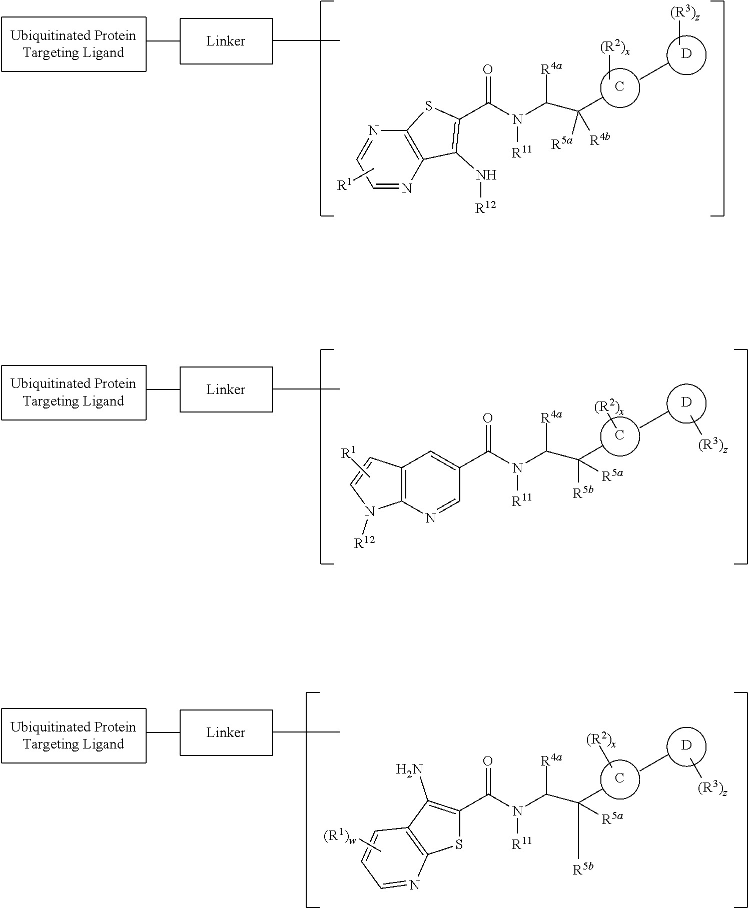

or a pharmaceutically acceptable salt thereof.

3. The compound of embodiment 29, wherein the compound is of Formula:

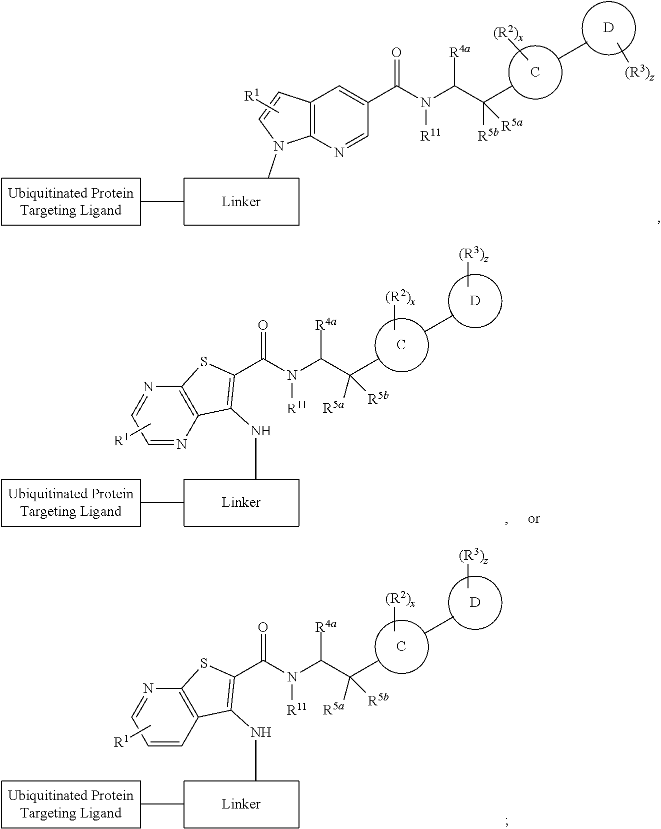

or a pharmaceutically acceptable salt thereof.

4. The compound of embodiment 1, wherein the compound is of Formula:

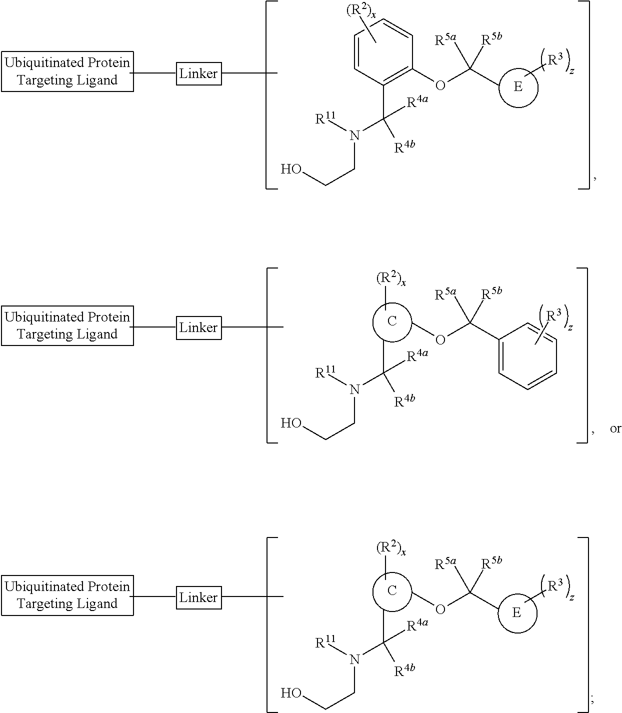

or a pharmaceutically acceptable salt thereof.

5. The compound of embodiment 31, wherein the compound is of Formula

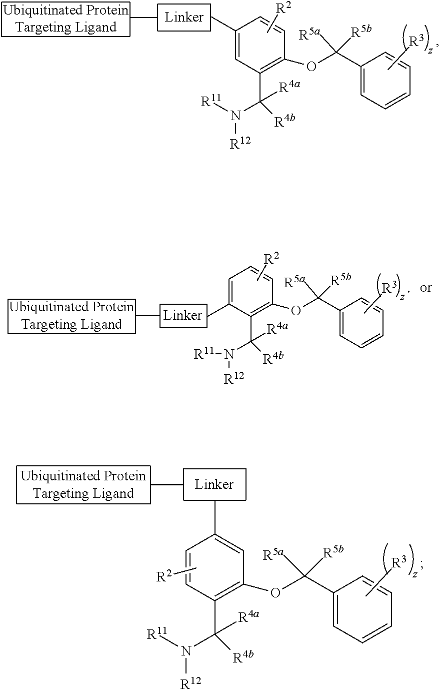

or a pharmaceutically acceptable salt thereof.

6. The compound of embodiment 1, wherein the compound is of Formula:

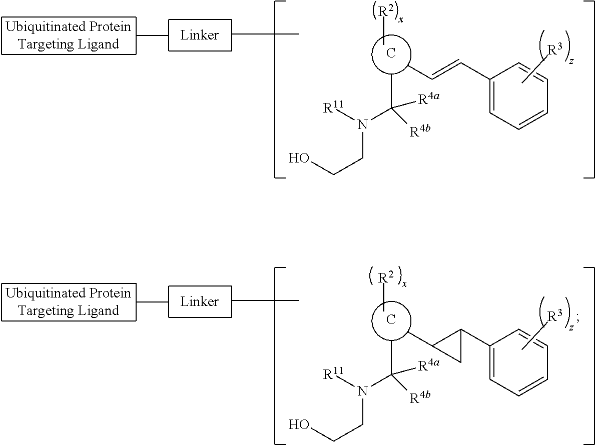

or a pharmaceutically acceptable salt thereof.

7. The compound of embodiment 1, wherein the compound is of Formula:

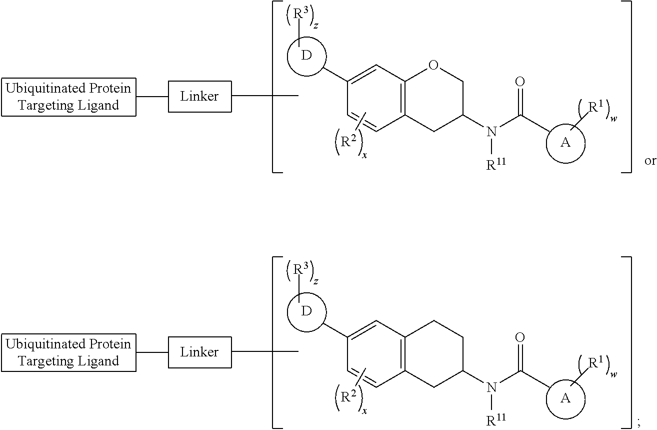

or pharmaceutically acceptable salt thereof.

8. The compound of embodiment 1, wherein the compound is of Formula:

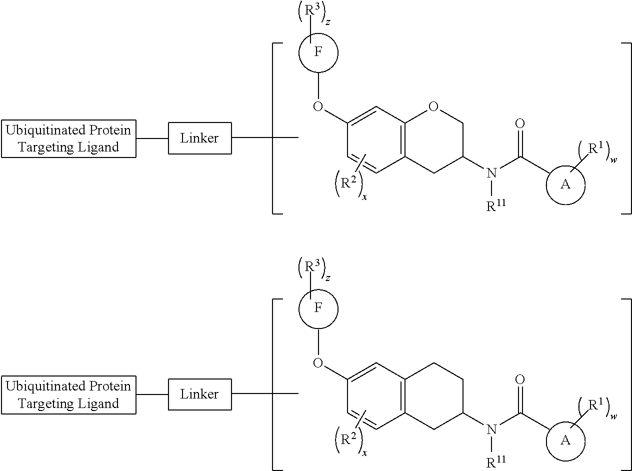

or a pharmaceutically acceptable salt thereof.

9. The compound of embodiment 1, wherein the compound is of Formula:

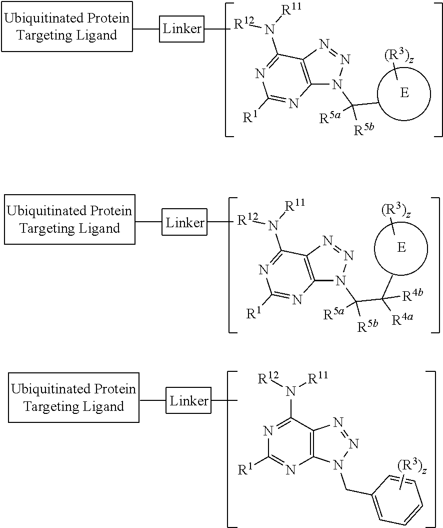

or a pharmaceutically acceptable salt thereof.

10. The compound of embodiment 36, wherein the compound is of Formula:

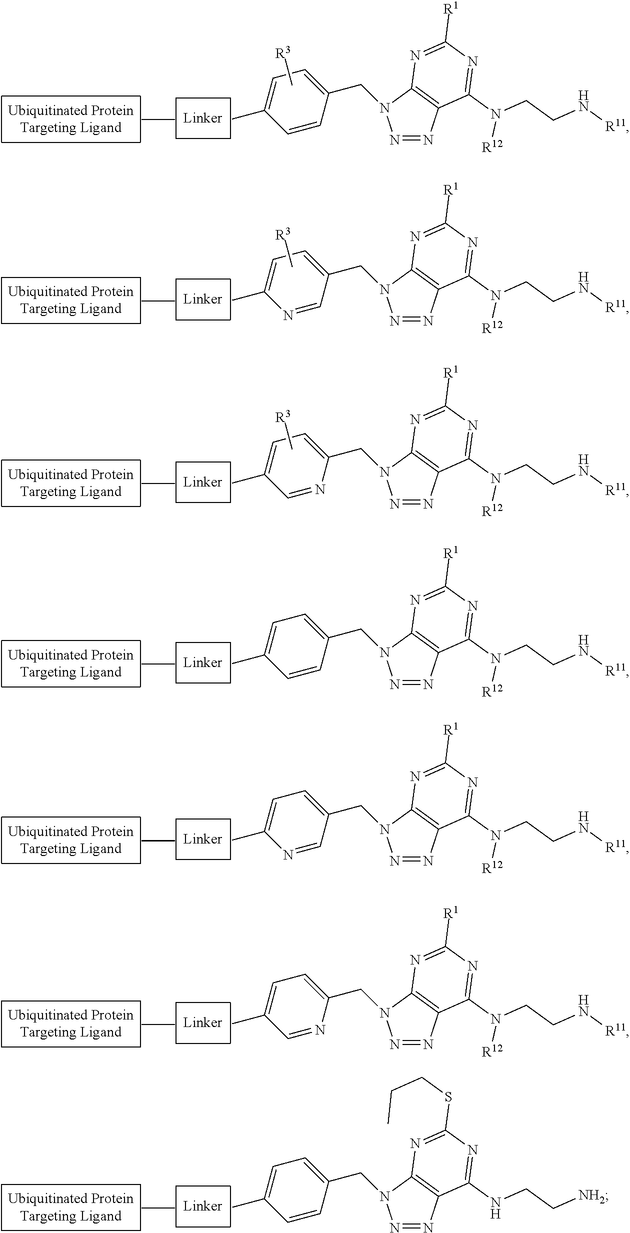

or a pharmaceutically acceptable salt thereof.

11. The compound of embodiment 1, wherein the compound is of Formula:

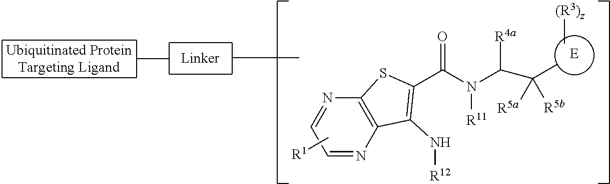

or a pharmaceutically acceptable salt thereof.

12. The compound of any one of embodiments 1-11, wherein

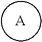

is an aryl group.

13. The compound of any one of embodiments 1-11, wherein

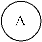

is a bicycle group.

14. The compound of any one of embodiments 1-11, wherein

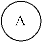

is

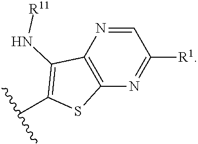

15. The compound of any one of embodiments 1-11, wherein

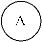

is

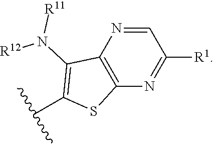

16. The compound of any one of embodiments 1-11, wherein

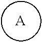

is

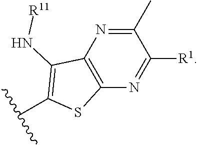

17. The compound of any one of embodiments 1-11, wherein

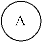

is

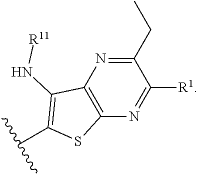

18. The compound of any one of embodiments 1-11, wherein

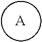

is

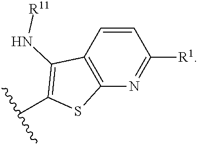

19. The compound of any one of embodiments 1-11, wherein

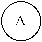

is

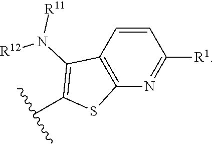

20. The compound of any one of embodiments 1-11, wherein

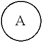

is

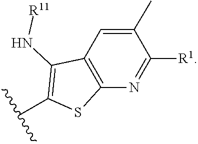

21. The compound of any one of embodiments 1-11, wherein

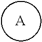

is

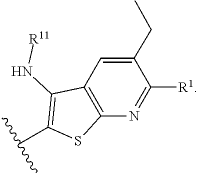

22. The compound of any one of embodiments 1-11, wherein

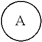

is

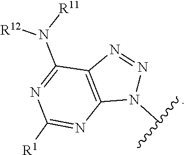

23. The compound of any one of embodiments 1-11, wherein

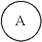

is

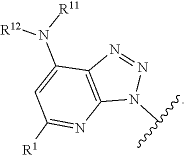

24. The compound of any one of embodiments 1-11, wherein

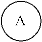

is

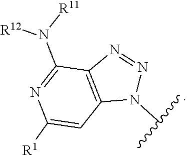

25. The compound of any one of embodiments 1-11, wherein

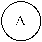

is

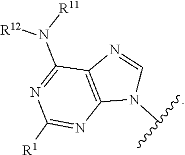

26. The compound of any one of embodiments 1-11, wherein

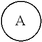

is

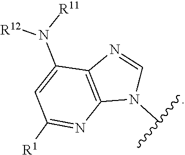

27. The compound of any one of embodiments 1-11, wherein

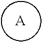

is

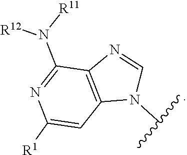

28. The compound of any one of embodiments 1-11, wherein

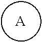

is

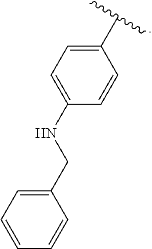 [SMILES: *c1ccc(NCc2ccccc2)cc1]

29. The compound of any one of embodiments 1-11, wherein

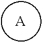

is

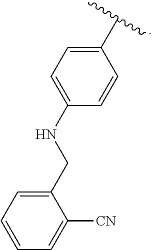 [SMILES: *c1ccc(NCc2ccccc2C#N)cc1]

30. The compound of any one of embodiments 1-11, wherein

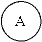

is

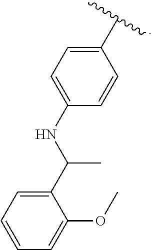 [SMILES: *c1ccc(NC(C)c2ccccc2OC)cc1]

31. The compound of any one of embodiments 1-11, wherein

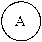

is

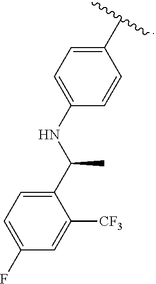 [SMILES: *c1ccc(N[C@@H](C)c2ccc(F)cc2C(F)(F)F)cc1]

32. The compound of any one of embodiments 1-11, wherein

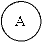

is

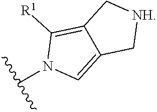

33. The compound of any one of embodiments 1-11, wherein

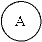

is

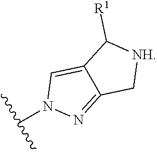

34. The compound of any one of embodiments 1-11, wherein

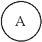

is

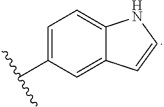 [SMILES: *c1ccc2[nH]ccc2c1]

35. The compound of any one of embodiments 1-11, wherein

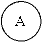

is

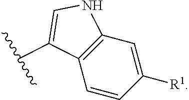

36. The compound of any one of embodiments 1-35, wherein

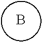

is a bicycle.

37. The compound of any one of embodiments 1-35, wherein

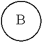

is a bicycle composed of two aryl rings.

38. The compound of any one of embodiments 1-35, wherein

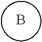

is a bicycle composed of one aryl ring and one heterocyclic ring.

39. The compound of any one of embodiments 1-35, wherein

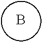

is

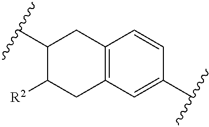

40. The compound of any one of embodiments 1-35, wherein

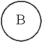

is

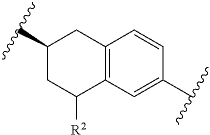

41. The compound of any one of embodiments 1-35, wherein

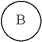

is

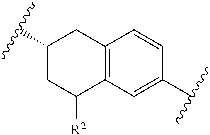

42. The compound of any one of embodiments 1-35, wherein

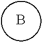

is

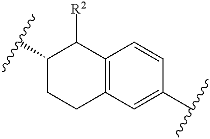

43. The compound of any one of embodiments 1-35, wherein

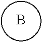

is

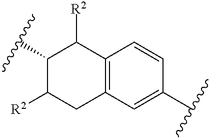

44. The compound of any one of embodiments 1-35, wherein

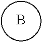

is

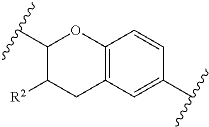

45. The compound of any one of embodiments 1-35, wherein

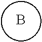

is

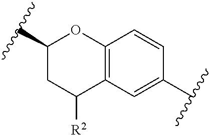

46. The compound of any one of embodiments 1-35, wherein

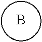

is

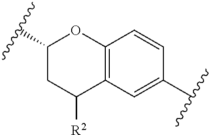

47. The compound of any one of embodiments 1-46, wherein

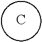

is a phenyl group.

48. The compound of any one of embodiments 1-46, wherein

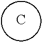

is

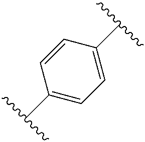 [SMILES: *c1ccc(*)cc1]

49. The compound of any one of embodiments 1-46, wherein

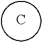

is

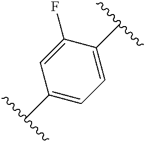 [SMILES: *c1ccc(*)c(F)c1]

50. The compound of any one of embodiments 1-46, wherein

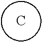

is

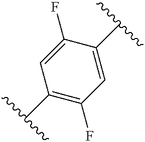 [SMILES: *c1cc(F)c(*)cc1F]

51. The compound of any one of embodiments 1-46, wherein

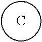

is

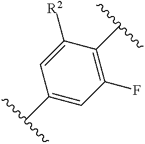

52. The compound of any one of embodiments 1-46, wherein

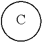

is

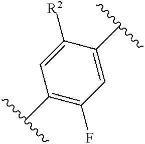

53. The compound of any one of embodiments 1-46, wherein

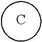

is

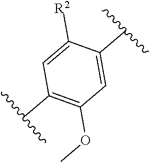

54. The compound of any one of embodiments 1-46, wherein

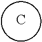

is

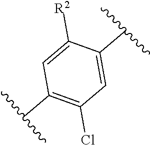

55. The compound of any one of embodiments 1-46, wherein

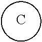

is

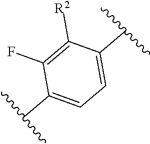

56. The compound of any one of embodiments 1-46, wherein

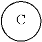

is

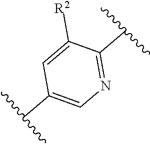

57. The compound of any one of embodiments 1-46, wherein

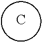

is

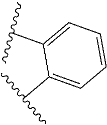 [SMILES: *c1ccccc1*]

58. The compound of any one of embodiments 1-46, wherein

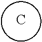

is

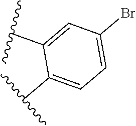 [SMILES: *c1ccc(Br)cc1*]

59. The compound of any one of embodiments 1-46, wherein

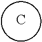

is

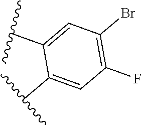 [SMILES: *c1cc(F)c(Br)cc1*]

60. The compound of any one of embodiments 1-46, wherein

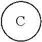

is

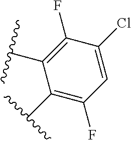 [SMILES: *c1c(F)cc(Cl)c(F)c1*]

61. The compound of any one of embodiments 1-46, wherein

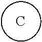

is

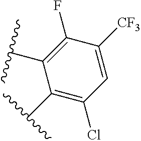 [SMILES: *c1c(Cl)cc(C(F)(F)F)c(F)c1*]

62. The compound of any one of embodiments 1-46, wherein

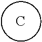

is

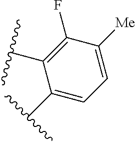 [SMILES: *c1ccc(C)c(F)c1*]

63. The compound of any one of embodiments 1-46, wherein

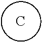

is

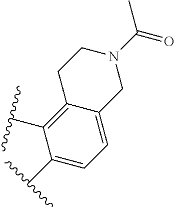 [SMILES: *c1ccc2c(c1*)CCN(C(C)=O)C2]

64. The compound of any one of embodiments 1-46, wherein

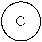

is

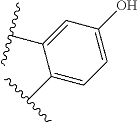 [SMILES: *c1ccc(O)cc1*]

65. The compound of any one of embodiments 1-46, wherein

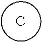

is

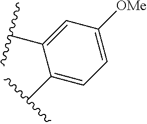 [SMILES: *c1ccc(OC)cc1*]

66. The compound of any one of embodiments 1-46, wherein

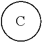

is

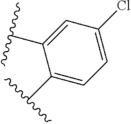 [SMILES: *c1ccc(Cl)cc1*]

67. The compound of any one of embodiments 1-46, wherein

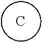

is

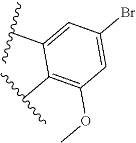 [SMILES: *c1cc(Br)cc(OC)c1*]

68. The compound of any one of embodiments 1-46, wherein

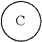

is

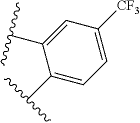 [SMILES: *c1ccc(C(F)(F)F)cc1*]

69. The compound of any one of embodiments 1-46, wherein

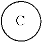

is

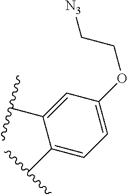 [SMILES: *c1ccc(OCCN=[N+]=[N-])cc1*]

70. The compound of any one of embodiments 1-46, wherein

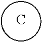

is

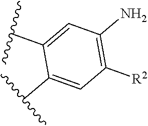

71. The compound of any one of embodiments 1-46, wherein

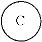

is

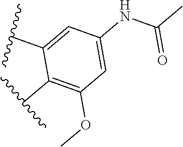 [SMILES: *c1cc(NC(C)=O)cc(OC)c1*]

72. The compound of any one of embodiments 1-46, wherein

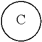

is

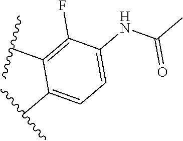 [SMILES: *c1ccc(NC(C)=O)c(F)c1*]

73. The compound of any one of embodiments 1-46, wherein

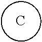

is

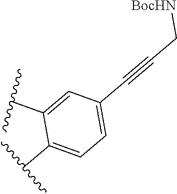 [SMILES: *c1ccc(C#CCNC(=O)OC(C)(C)C)cc1*]

74. The compound of any one of embodiments 1-46, wherein

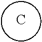

is

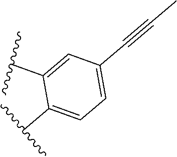 [SMILES: *c1ccc(C#CC)cc1*]

75. The compound of any one of embodiments 1-46, wherein

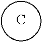

is

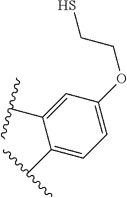 [SMILES: *c1ccc(OCCS)cc1*]

76. The compound of any one of embodiments 1-46, wherein

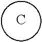

is

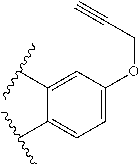 [SMILES: *c1ccc(OCC#C)cc1*]

77. The compound of any one of embodiments 1-46, wherein

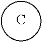

is

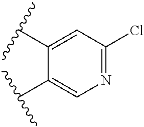 [SMILES: *c1cnc(Cl)cc1*]

78. The compound of any one of embodiments 1-46, wherein

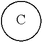

is

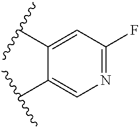 [SMILES: *c1cnc(F)cc1*]

79. The compound of any one of embodiments 1-46, wherein

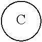

is

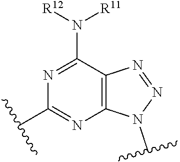

80. The compound of any one of embodiments 1-46, wherein

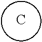

### is N

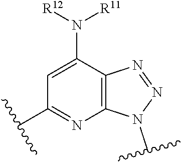

81. The compound of any one of embodiments 1-46, wherein

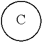

is

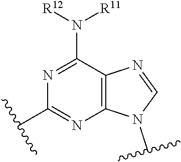

82. The compound of any one of embodiments 1-81, wherein

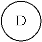

is a heterocycle.

83. The compound of any one of embodiments 1-81, wherein

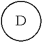

is a substituted piperazine.

84. The compound of any one of embodiments 1-81, wherein

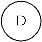

is substituted bicyclic piperazine.

85. The compound of any one of embodiments 1-81, wherein

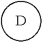

is

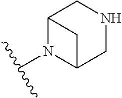 [SMILES: *N1C2CNCC1C2]

86. The compound of any one of embodiments 1-81, wherein

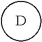

is

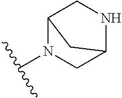 [SMILES: *N1CC2CC1CN2]

87. The compound of any one of embodiments 1-81, wherein

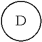

is

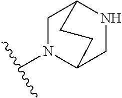 [SMILES: *N1CC2CCC1CN2]

88. The compound of any one of embodiments 1-81, wherein

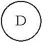

is

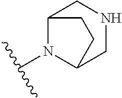 [SMILES: *N1C2CCC1CNC2]

89. The compound of any one of embodiments 1-81, wherein

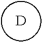

is

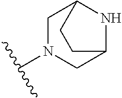 [SMILES: *N1CC2CCC(C1)N2]

90. The compound of any one of embodiments 1-81, wherein

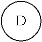

is

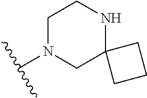 [SMILES: *N1CCNC2(CCC2)C1]

91. The compound of any one of embodiments 1-81, wherein

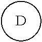

is

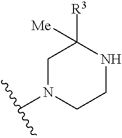

92. The compound of any one of embodiments 1-81, wherein

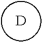

is

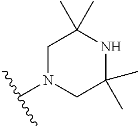 [SMILES: *N1CC(C)(C)NC(C)(C)C1]

93. The compound of any one of embodiments 1-81, wherein

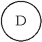

is

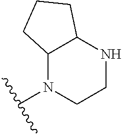 [SMILES: *N1CCNC2CCCC21]

94. The compound of any one of embodiments 1-81, wherein

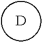

is

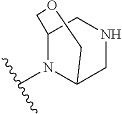 [SMILES: *N1C2CNCC1COC2]

95. The compound of any one of embodiments 1-81, wherein

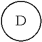

is

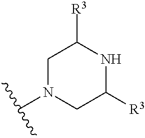

96. The compound of any one of embodiments 1-81, wherein

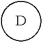

is

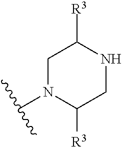

97. The compound of any one of embodiments 1-81, wherein

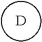

### is Me

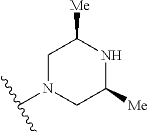 [SMILES: *N1C[C@@H](C)N[C@@H](C)C1]

98. The compound of any one of embodiments 1-81, wherein

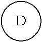

is

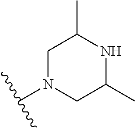 [SMILES: *N1CC(C)NC(C)C1]

99. The compound of any one of embodiments 1-81, wherein

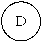

is

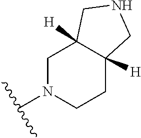 [SMILES: *N1CC[C@H]2CNC[C@H]2C1]

100. The compound of any one of embodiments 1-81, wherein

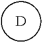

is

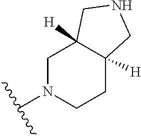 [SMILES: *N1CC[C@@H]2CNC[C@H]2C1]

101. The compound of any one of embodiments 1-81, wherein

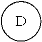

is

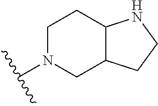 [SMILES: *N1CCC2NCCC2C1]

102. The compound of any one of embodiments 1-81, wherein

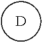

is

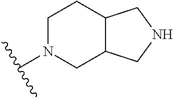 [SMILES: *N1CCC2CNCC2C1]

103. The compound of any one of embodiments 1-81, wherein

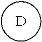

is

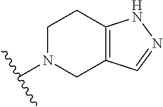 [SMILES: *N1CCc2[nH]ncc2C1]

104. The compound of any one of embodiments 1-81, wherein

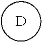

is

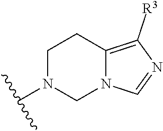

105. The compound of any one of embodiments 1-104, wherein

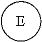

is an aryl group.

106. The compound of any one of embodiments 1-104, wherein

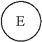

is a phenyl group.

107. The compound of any one of embodiments 1-104, wherein

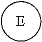

is

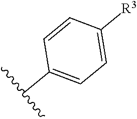

108. The compound of any one of embodiments 1-104, wherein

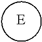

is

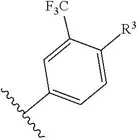

109. The compound of any one of embodiments 1-104, wherein

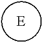

is

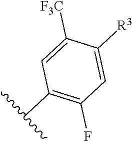

110. The compound of any one of embodiments 1-104, wherein

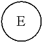

is

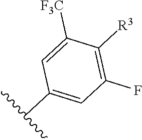

111. The compound of any one of embodiments 1-104, wherein

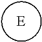

is

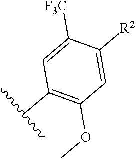

112. The compound of any one of embodiments 1-104, wherein

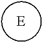

is

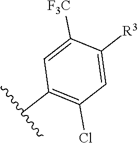

113. The compound of any one of embodiments 1-104, wherein

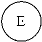

is

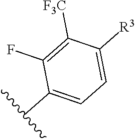

114. The compound of any one of embodiments 1-104, wherein

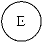

is

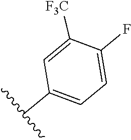 [SMILES: *c1ccc(F)c(C(F)(F)F)c1]

115. The compound of any one of embodiments 1-104, wherein

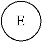

is

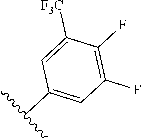 [SMILES: *c1cc(F)c(F)c(C(F)(F)F)c1]

116. The compound of any one of embodiments 1-104, wherein

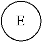

is

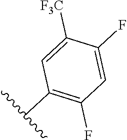 [SMILES: *c1cc(C(F)(F)F)c(F)cc1F]

117. The compound of any one of embodiments 1-104, wherein

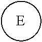

is

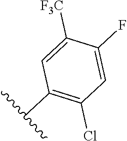 [SMILES: *c1cc(C(F)(F)F)c(F)cc1Cl]

118. The compound of any one of embodiments 1-104, wherein

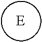

is

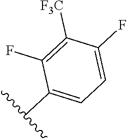 [SMILES: *c1ccc(F)c(C(F)(F)F)c1F]

119. The compound of any one of embodiments 1-104, wherein

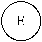

is

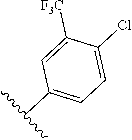 [SMILES: *c1ccc(Cl)c(C(F)(F)F)c1]

120. The compound of any one of embodiments 1-104, wherein

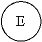

is

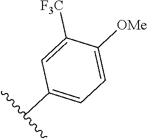 [SMILES: *c1ccc(OC)c(C(F)(F)F)c1]

121. The compound of any one of embodiments 1-104, wherein

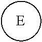

is

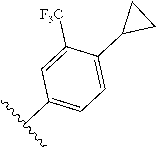 [SMILES: *c1ccc(C2CC2)c(C(F)(F)F)c1]

122. The compound of any one of embodiments 1-104, wherein

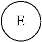

is

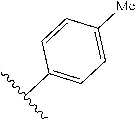 [SMILES: *c1ccc(C)cc1]

123. The compound of any one of embodiments 1-104, wherein

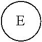

is

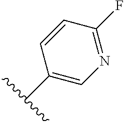 [SMILES: *c1ccc(F)nc1]

124. The compound of any one of embodiments 1-104 wherein

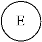

is

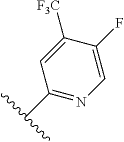 [SMILES: *c1cc(C(F)(F)F)c(F)cn1]

125. The compound of any one of embodiments 1-104, wherein

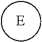

is

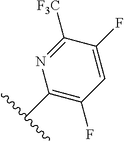 [SMILES: *c1nc(C(F)(F)F)c(F)cc1F]

126. The compound of any one of embodiments 1-104, wherein

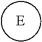

is

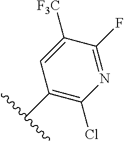 [SMILES: *c1cc(C(F)(F)F)c(F)nc1Cl]

127. The compound of any one of embodiments 1-126, wherein a R¹ is hydrogen.

128. The compound of any one of embodiments 1-126, wherein one R¹ is hydrogen.

129. The compound of any one of embodiments 1-126, wherein all R¹ groups are hydrogen.

130. The compound of any one of embodiments 1-126, wherein a R¹ is halogen.

131. The compound of any one of embodiments 1-126, wherein one R¹ is halogen.

132. The compound of any one of embodiments 1-126, wherein a R¹ is alkyl optionally substituted with 1, 2, 3, or 4 substituents selected from R²¹.

133. The compound of any one of embodiments 1-126, wherein one R¹ is alkyl optionally substituted with 1, 2, 3, or 4 substituents selected from R²¹.

134. The compound of any one of embodiments 1-126, wherein a R¹ is haloalkyl optionally substituted with 1, 2, 3, or 4 substituents selected from R²¹.

135. The compound of any one of embodiments 1-126, wherein one R¹ is haloalkyl optionally substituted with 1, 2, 3, or 4 substituents selected from R²¹.

136. The compound of any one of embodiments 1-126, wherein a R¹ is alkenyl optionally substituted with 1, 2, 3, or 4 substituents selected from R²¹.

137. The compound of any one of embodiments 1-126, wherein one R¹ is alkenyl optionally substituted with 1, 2, 3, or 4 substituents selected from R²¹.

138. The compound of any one of embodiments 1-126, wherein a R¹ is alkynyl optionally substituted with 1, 2, 3, or 4 substituents selected from R²¹.

139. The compound of any one of embodiments 1-126, wherein one R¹ is alkynyl optionally substituted with 1, 2, 3, or 4 substituents selected from R²¹.

140. The compound of any one of embodiments 1-126, wherein a R¹ is heterocycle optionally substituted with 1, 2, 3, or 4 substituents selected from R²¹.

141. The compound of any one of embodiments 1-126, wherein one R¹ is heterocycle optionally substituted with 1, 2, 3, or 4 substituents selected from R²¹.

142. The compound of any one of embodiments 1-126, wherein a R¹ is aryl optionally substituted with 1, 2, 3, or 4 substituents selected from R²¹.

143. The compound of any one of embodiments 1-126, wherein one R¹ is aryl optionally substituted with 1, 2, 3, or 4 substituents selected from R²¹.

144. The compound of any one of embodiments 1-126, wherein a R¹ is heteroaryl optionally substituted with 1, 2, 3, or 4 substituents selected from R²¹.

145. The compound of any one of embodiments 1-126, wherein one R¹ is heteroaryl optionally substituted with 1, 2, 3, or 4 substituents selected from R²¹.

146. The compound of any one of embodiments 1-126, wherein a R¹ is cyano.

147. The compound of any one of embodiments 1-126, wherein one R¹ is cyano.

148. The compound of any one of embodiments 1-126, wherein a R¹ is nitro.

149. The compound of any one of embodiments 1-126, wherein one R¹ is nitro.

150. The compound of any one of embodiments 1-126, wherein a R¹ is —C(O)R¹⁰.

151. The compound of any one of embodiments 1-126, wherein one R¹ is —C(O)R¹⁰.

152. The compound of any one of embodiments 1-126, wherein a R¹ is —OC(O)R¹⁰.

153. The compound of any one of embodiments 1-126, wherein one R¹ is —OC(O)R¹⁰.

154. The compound of any one of embodiments 1-126, wherein a R¹ is —NR¹¹C(O)R¹⁰.

155. The compound of any one of embodiments 1-126, wherein one R¹ is —NR¹¹C(O)R¹⁰.

156. The compound of any one of embodiments 1-126, wherein a R¹ is —OR¹¹.

157. The compound of any one of embodiments 1-126, wherein one R¹ is —OR¹¹.

158. The compound of any one of embodiments 1-126, wherein a R¹ is —NR¹¹R¹².

159. The compound of any one of embodiments 1-126, wherein one R¹ is —NR¹¹R¹².

160. The compound of any one of embodiments 1-126, wherein a R¹ is —S(O)R¹⁰.

161. The compound of any one of embodiments 1-126, wherein one R¹ is —S(O)R¹⁰.

162. The compound of any one of embodiments 1-126, wherein a R¹ is —S(O)₂R¹⁰.

163. The compound of any one of embodiments 1-126, wherein one R¹ is —S(O)₂R¹⁰.

164. The compound of any one of embodiments 1-126, wherein a R¹ is —OS(O)R¹⁰.

165. The compound of any one of embodiments 1-126, wherein one R¹ is —OS(O)R¹⁰.

166. The compound of any one of embodiments 1-126, wherein a R¹ is —OS(O)₂R¹⁰.

167. The compound of any one of embodiments 1-126, wherein one R¹ is —OS(O)₂R¹⁰.

168. The compound of any one of embodiments 1-126, wherein a R¹ is —NR¹¹S(O)R¹⁰.

169. The compound of any one of embodiments 1-126, wherein one R¹ is —NR¹¹S(O)R¹⁰.

170. The compound of any one of embodiments 1-126, wherein a R¹ is —NR¹¹S(O)₂R¹⁰.

171. The compound of any one of embodiments 1-126, wherein one R¹ is —NR¹¹S(O)₂R¹⁰.

172. The compound of any one of embodiments 1-126, wherein a R¹ is —SR¹¹.

173. The compound of any one of embodiments 1-126, wherein one R¹ is —SR¹¹.

174. The compound of any one of embodiments 1-173, wherein a R² is hydrogen.

175. The compound of any one of embodiments 1-173, wherein one R² is hydrogen.

176. The compound of any one of embodiments 1-173, wherein all R² groups are hydrogen.

177. The compound of any one of embodiments 1-173, wherein a R² is halogen.

178. The compound of any one of embodiments 1-173, wherein one R² is halogen.

179. The compound of any one of embodiments 1-173, wherein a R² is alkyl optionally substituted with 1, 2, 3, or 4 substituents selected from R²².

180. The compound of any one of embodiments 1-173, wherein one R² is alkyl optionally substituted with 1, 2, 3, or 4 substituents selected from R²².

181. The compound of any one of embodiments 1-173, wherein a R² is haloalkyl optionally substituted with 1, 2, 3, or 4 substituents selected from R²².

182. The compound of any one of embodiments 1-173, wherein one R² is haloalkyl optionally substituted with 1, 2, 3, or 4 substituents selected from R²².

183. The compound of any one of embodiments 1-173, wherein a R² is alkenyl optionally substituted with 1, 2, 3, or 4 substituents selected from R²².

184. The compound of any one of embodiments 1-173, wherein one R² is alkenyl optionally substituted with 1, 2, 3, or 4 substituents selected from R²².

185. The compound of any one of embodiments 1-173, wherein a R² is alkynyl optionally substituted with 1, 2, 3, or 4 substituents selected from R²².

186. The compound of any one of embodiments 1-173, wherein one R² is alkynyl optionally substituted with 1, 2, 3, or 4 substituents selected from R²².

187. The compound of any one of embodiments 1-173, wherein a R² is heterocycle optionally substituted with 1, 2, 3, or 4 substituents selected from R²².

188. The compound of any one of embodiments 1-173, wherein one R² is heterocycle optionally substituted with 1, 2, 3, or 4 substituents selected from R²².

189. The compound of any one of embodiments 1-173, wherein a R² is aryl optionally substituted with 1, 2, 3, or 4 substituents selected from R²².

190. The compound of any one of embodiments 1-173, wherein one R² is aryl optionally substituted with 1, 2, 3, or 4 substituents selected from R²².

191. The compound of any one of embodiments 1-173, wherein a R² is heteroaryl optionally substituted with 1, 2, 3, or 4 substituents selected from R²².

192. The compound of any one of embodiments 1-173, wherein one R² is heteroaryl optionally substituted with 1, 2, 3, or 4 substituents selected from R²².

193. The compound of any one of embodiments 1-173, wherein a R² is cyano.

194. The compound of any one of embodiments 1-173, wherein one R² is cyano.

195. The compound of any one of embodiments 1-173, wherein a R² is nitro.

196. The compound of any one of embodiments 1-173, wherein one R² is nitro.

197. The compound of any one of embodiments 1-173, wherein a R² is —C(O)R¹⁰.

198. The compound of any one of embodiments 1-173, wherein one R² is —C(O)R¹⁰.

199. The compound of any one of embodiments 1-173, wherein a R² is —OC(O)R¹⁰.

200. The compound of any one of embodiments 1-173, wherein one R² is —OC(O)R¹⁰.

201. The compound of any one of embodiments 1-173, wherein a R² is —NR¹¹C(O)R¹⁰.

202. The compound of any one of embodiments 1-173, wherein one R² is —NR¹¹C(O)R¹⁰.

203. The compound of any one of embodiments 1-173, wherein a R² is —OR¹¹.

204. The compound of any one of embodiments 1-173, wherein one R² is —OR¹¹.

205. The compound of any one of embodiments 1-173, wherein a R² is —NR¹¹R¹².

206. The compound of any one of embodiments 1-173, wherein one R² is —NR¹¹R¹².

207. The compound of any one of embodiments 1-173, wherein a R² is —S(O)R¹⁰.

208. The compound of any one of embodiments 1-173, wherein one R² is —S(O)R¹⁰.

209. The compound of any one of embodiments 1-173, wherein a R² is —S(O)₂R¹⁰.

210. The compound of any one of embodiments 1-173, wherein one R² is —S(O)₂R¹⁰.

211. The compound of any one of embodiments 1-173, wherein a R² is —OS(O)R¹⁰.

212. The compound of any one of embodiments 1-173, wherein one R² is —OS(O)R¹⁰.

213. The compound of any one of embodiments 1-173, wherein a R² is —OS(O)₂R¹⁰.

214. The compound of any one of embodiments 1-173, wherein one R² is —OS(O)₂R¹⁰.

215. The compound of any one of embodiments 1-173, wherein a R² is —NR¹¹S(O)R¹⁰.

216. The compound of any one of embodiments 1-173, wherein one R² is —NR¹¹S(O)R¹⁰.

217. The compound of any one of embodiments 1-173, wherein a R² is —NR¹¹S(O)₂R¹⁰.

218. The compound of any one of embodiments 1-173, wherein one R² is —NR¹¹S(O)₂R¹⁰.

219. The compound of any one of embodiments 1-173, wherein a R² is —SR¹¹.

220. The compound of any one of embodiments 1-173, wherein one R² is —SR¹¹.

221. The compound of any one of embodiments 1-220, wherein a R³ is hydrogen.

222. The compound of any one of embodiments 1-220, wherein one R³ is hydrogen.

223. The compound of any one of embodiments 1-220, wherein all R³ groups are hydrogen.

224. The compound of any one of embodiments 1-220, wherein a R³ is halogen.

225. The compound of any one of embodiments 1-220, wherein one R³ is halogen.

226. The compound of any one of embodiments 1-220, wherein a R³ is alkyl optionally substituted with 1, 2, 3, or 4 substituents selected from R²³.

227. The compound of any one of embodiments 1-220, wherein one R³ is alkyl optionally substituted with 1, 2, 3, or 4 substituents selected from R²³.

228. The compound of any one of embodiments 1-220, wherein a R³ is haloalkyl optionally substituted with 1, 2, 3, or 4 substituents selected from R²³.

229. The compound of any one of embodiments 1-220, wherein one R³ is haloalkyl optionally substituted with 1, 2, 3, or 4 substituents selected from R²³.

230. The compound of any one of embodiments 1-220, wherein a R³ is alkenyl optionally substituted with 1, 2, 3, or 4 substituents selected from R²³.

231. The compound of any one of embodiments 1-220, wherein one R³ is alkenyl optionally substituted with 1, 2, 3, or 4 substituents selected from R²³.

232. The compound of any one of embodiments 1-220, wherein a R³ is alkynyl optionally substituted with 1, 2, 3, or 4 substituents selected from R²³.

233. The compound of any one of embodiments 1-220, wherein one R³ is alkynyl optionally substituted with 1, 2, 3, or 4 substituents selected from R²³.

234. The compound of any one of embodiments 1-220, wherein a R³ is heterocycle optionally substituted with 1, 2, 3, or 4 substituents selected from R²³.

235. The compound of any one of embodiments 1-220, wherein one R³ is heterocycle optionally substituted with 1, 2, 3, or 4 substituents selected from R²³

236. The compound of any one of embodiments 1-220, wherein a R³ is aryl optionally substituted with 1, 2, 3, or 4 substituents selected from R²³.

237. The compound of any one of embodiments 1-220, wherein one R³ is aryl optionally substituted with 1, 2, 3, or 4 substituents selected from R²³.

238. The compound of any one of embodiments 1-220, wherein a R³ is heteroaryl optionally substituted with 1, 2, 3, or 4 substituents selected from R²³.

239. The compound of any one of embodiments 1-220, wherein one R³ is heteroaryl optionally substituted with 1, 2, 3, or 4 substituents selected from R²³.

240. The compound of any one of embodiments 1-220, wherein a R³ is cyano.

241. The compound of any one of embodiments 1-220, wherein one R³ is cyano.

242. The compound of any one of embodiments 1-220, wherein a R³ is nitro.

243. The compound of any one of embodiments 1-220, wherein one R³ is nitro.

244. The compound of any one of embodiments 1-220, wherein a R³ is —C(O)R¹⁰.

245. The compound of any one of embodiments 1-220, wherein one R³ is —C(O)R¹⁰.

246. The compound of any one of embodiments 1-220, wherein a R³ is —OC(O)R¹⁰.

247. The compound of any one of embodiments 1-220, wherein one R³ is —OC(O)R¹⁰.

248. The compound of any one of embodiments 1-220, wherein a R³ is —NR¹¹C(O)R¹⁰.

249. The compound of any one of embodiments 1-220, wherein one R³ is —NR¹¹C(O)R¹⁰.

250. The compound of any one of embodiments 1-220, wherein a R³ is —OR¹¹.

251. The compound of any one of embodiments 1-220, wherein one R³ is —OR¹¹.

252. The compound of any one of embodiments 1-220, wherein a R³ is —NR₁₁R¹².

253. The compound of any one of embodiments 1-220, wherein one R³ is —NR₁₁R¹².

254. The compound of any one of embodiments 1-220, wherein a R³ is —S(O)R¹⁰.

255. The compound of any one of embodiments 1-220, wherein one R³ is —S(O)R¹⁰.

256. The compound of any one of embodiments 1-220, wherein a R³ is —S(O)₂R¹⁰.

257. The compound of any one of embodiments 1-220, wherein one R³ is —S(O)₂R¹⁰.

258. The compound of any one of embodiments 1-220, wherein a R³ is —OS(O)R¹⁰.

259. The compound of any one of embodiments 1-220, wherein one R³ is —OS(O)R¹⁰.

260. The compound of any one of embodiments 1-220, wherein a R³ is —OS(O)₂R¹⁰.

261. The compound of any one of embodiments 1-220, wherein one R³ is —OS(O)₂R¹⁰.

262. The compound of any one of embodiments 1-220, wherein a R³ is —NR¹¹S(O)R¹⁰.

263. The compound of any one of embodiments 1-220, wherein one R³ is —NR¹¹S(O)R¹⁰.

264. The compound of any one of embodiments 1-220, wherein a R³ is —NR¹¹S(O)₂R¹⁰.

265. The compound of any one of embodiments 1-220, wherein one R³ is —NR¹¹S(O)₂R¹⁰.

266. The compound of any one of embodiments 1-220, wherein a R³ is —SR¹¹.

267. The compound of any one of embodiments 1-220, wherein one R³ is —SR¹¹.

268. The compound of any one of embodiments 1-267, wherein a R⁴ is hydrogen.

269. The compound of any one of embodiments 1-267, wherein one R⁴ is hydrogen.

270. The compound of any one of embodiments 1-267, wherein all R⁴ groups are hydrogen.

271. The compound of any one of embodiments 1-267, wherein a R⁴ is halogen.

272. The compound of any one of embodiments 1-267, wherein one R⁴ is halogen.

273. The compound of any one of embodiments 1-267, wherein a R⁴ is alkyl optionally substituted with 1, 2, 3, or 4 substituents selected from R²⁴.

274. The compound of any one of embodiments 1-267, wherein one R⁴ is alkyl optionally substituted with 1, 2, 3, or 4 substituents selected from R²⁴.

275. The compound of any one of embodiments 1-267, wherein a R⁴ is haloalkyl optionally substituted with 1, 2, 3, or 4 substituents selected from R²⁴.

276. The compound of any one of embodiments 1-267, wherein one R⁴ is haloalkyl optionally substituted with 1, 2, 3, or 4 substituents selected from R²⁴.

277. The compound of any one of embodiments 1-267, wherein a R⁴ is alkenyl optionally substituted with 1, 2, 3, or 4 substituents selected from R²⁴.

278. The compound of any one of embodiments 1-267, wherein one R⁴ is alkenyl optionally substituted with 1, 2, 3, or 4 substituents selected from R²⁴.

279. The compound of any one of embodiments 1-267, wherein a R⁴ is alkynyl optionally substituted with 1, 2, 3, or 4 substituents selected from R²⁴.

280. The compound of any one of embodiments 1-267, wherein one R⁴ is alkynyl optionally substituted with 1, 2, 3, or 4 substituents selected from R²⁴.

281. The compound of any one of embodiments 1-267, wherein a R⁴ is heterocycle optionally substituted with 1, 2, 3, or 4 substituents selected from R²⁴.

282. The compound of any one of embodiments 1-267, wherein one R⁴ is heterocycle optionally substituted with 1, 2, 3, or 4 substituents selected from R²⁴.

283. The compound of any one of embodiments 1-267, wherein a R⁴ is aryl optionally substituted with 1, 2, 3, or 4 substituents selected from R²⁴.

284. The compound of any one of embodiments 1-267, wherein one R⁴ is aryl optionally substituted with 1, 2, 3, or 4 substituents selected from R²⁴.

285. The compound of any one of embodiments 1-267, wherein a R⁴ is heteroaryl optionally substituted with 1, 2, 3, or 4 substituents selected from R²⁴.

286. The compound of any one of embodiments 1-267, wherein one R⁴ is heteroaryl optionally substituted with 1, 2, 3, or 4 substituents selected from R²⁴.

287. The compound of any one of embodiments 1-267, wherein a R⁴ is cyano.

288. The compound of any one of embodiments 1-267, wherein one R⁴ is cyano.

289. The compound of any one of embodiments 1-267, wherein a R⁴ is nitro.

290. The compound of any one of embodiments 1-267, wherein one R⁴ is nitro.

291. The compound of any one of embodiments 1-267, wherein a R⁴ is —C(O)R¹⁰.

292. The compound of any one of embodiments 1-267, wherein one R⁴ is —C(O)R¹⁰.

293. The compound of any one of embodiments 1-267, wherein a R⁴ is —OC(O)R¹⁰.

294. The compound of any one of embodiments 1-267, wherein one R⁴ is —OC(O)R¹⁰.

295. The compound of any one of embodiments 1-267, wherein a R⁴ is —NR¹¹C(O)R¹⁰.

296. The compound of any one of embodiments 1-267, wherein one R⁴ is —NR¹¹C(O)R¹⁰.

297. The compound of any one of embodiments 1-267, wherein a R⁴ is —OR¹¹.

298. The compound of any one of embodiments 1-267, wherein one R⁴ is —OR¹¹.

299. The compound of any one of embodiments 1-267, wherein a R⁴ is —NR¹¹R¹².

300. The compound of any one of embodiments 1-267, wherein one R⁴ is —NR¹¹R¹².

301. The compound of any one of embodiments 1-267, wherein a R⁴ is —S(O)R¹⁰.

302. The compound of any one of embodiments 1-267, wherein one R⁴ is —S(O)R¹⁰.

303. The compound of any one of embodiments 1-267, wherein a R⁴ is —S(O)₂R¹⁰.

304. The compound of any one of embodiments 1-267, wherein one R⁴ is —S(O)₂R¹⁰.

305. The compound of any one of embodiments 1-267, wherein a R⁴ is —OS(O)R¹⁰.

306. The compound of any one of embodiments 1-267, wherein one R⁴ is —OS(O)R¹⁰.

307. The compound of any one of embodiments 1-267, wherein a R⁴ is —OS(O)₂R¹⁰.

308. The compound of any one of embodiments 1-267, wherein one R⁴ is —OS(O)₂R¹⁰.

309. The compound of any one of embodiments 1-267, wherein a R⁴ is —NR¹¹S(O)R¹⁰.

310. The compound of any one of embodiments 1-267, wherein one R⁴ is —NR¹¹S(O)R¹⁰.

311. The compound of any one of embodiments 1-267, wherein a R⁴ is —NR¹¹S(O)₂R¹⁰.

312. The compound of any one of embodiments 1-267, wherein one R⁴ is —NR¹¹S(O)₂R¹⁰.

313. The compound of any one of embodiments 1-267, wherein a R⁴ is —SR¹¹.

314. The compound of any one of embodiments 1-267, wherein one R⁴ is —SR¹¹.

315. The compound of any one of embodiments 1-314, wherein a R⁵ is hydrogen.

316. The compound of any one of embodiments 1-314, wherein one R⁵ is hydrogen.

317. The compound of any one of embodiments 1-314, wherein all R⁵ groups are hydrogen.

318. The compound of any one of embodiments 1-314, wherein a R⁵ is halogen.

319. The compound of any one of embodiments 1-314, wherein one R⁵ is halogen.

320. The compound of any one of embodiments 1-314, wherein a R⁵ is alkyl optionally substituted with 1, 2, 3, or 4 substituents selected from R²⁵.

321. The compound of any one of embodiments 1-314, wherein one R⁵ is alkyl optionally substituted with 1, 2, 3, or 4 substituents selected from R²⁵.

322. The compound of any one of embodiments 1-314, wherein a R⁵ is haloalkyl optionally substituted with 1, 2, 3, or 4 substituents selected from R²⁵.

323. The compound of any one of embodiments 1-314, wherein one R⁵ is haloalkyl optionally substituted with 1, 2, 3, or 4 substituents selected from R²⁵.

324. The compound of any one of embodiments 1-314, wherein a R⁵ is alkenyl optionally substituted with 1, 2, 3, or 4 substituents selected from R²⁵.

325. The compound of any one of embodiments 1-314, wherein one R⁵ is alkenyl optionally substituted with 1, 2, 3, or 4 substituents selected from R²⁵.

326. The compound of any one of embodiments 1-314, wherein a R⁵ is alkynyl optionally substituted with 1, 2, 3, or 4 substituents selected from R²⁵.

327. The compound of any one of embodiments 1-314, wherein one R⁵ is alkynyl optionally substituted with 1, 2, 3, or 4 substituents selected from R²⁵.

328. The compound of any one of embodiments 1-314, wherein a R⁵ is heterocycle optionally substituted with 1, 2, 3, or 4 substituents selected from R²⁵.

329. The compound of any one of embodiments 1-314, wherein one R⁵ is heterocycle optionally substituted with 1, 2, 3, or 4 substituents selected from R²⁵.

330. The compound of any one of embodiments 1-314, wherein a R⁵ is aryl optionally substituted with 1, 2, 3, or 4 substituents selected from R²⁵.

331. The compound of any one of embodiments 1-314, wherein one R⁵ is aryl optionally substituted with 1, 2, 3, or 4 substituents selected from R²⁵.

332. The compound of any one of embodiments 1-314, wherein a R⁵ is heteroaryl optionally substituted with 1, 2, 3, or 4 substituents selected from R²⁵.

333. The compound of any one of embodiments 1-314, wherein one R⁵ is heteroaryl optionally substituted with 1, 2, 3, or 4 substituents selected from R²⁵.

334. The compound of any one of embodiments 1-314, wherein a R⁵ is cyano.

335. The compound of any one of embodiments 1-314, wherein one R⁵ is cyano.

336. The compound of any one of embodiments 1-314, wherein a R⁵ is nitro.

337. The compound of any one of embodiments 1-314, wherein one R⁵ is nitro.

338. The compound of any one of embodiments 1-314, wherein a R⁵ is —C(O)R¹⁰.

339. The compound of any one of embodiments 1-314, wherein one R⁵ is —C(O)R¹⁰.

340. The compound of any one of embodiments 1-314, wherein a R⁵ is —OC(O)R¹⁰.

341. The compound of any one of embodiments 1-314, wherein one R⁵ is —OC(O)R¹⁰.

342. The compound of any one of embodiments 1-314, wherein a R⁵ is —NR¹¹C(O)R¹⁰.

343. The compound of any one of embodiments 1-314, wherein one R⁵ is —NR¹¹C(O)R¹⁰.

344. The compound of any one of embodiments 1-314, wherein a R⁵ is —OR¹¹.

345. The compound of any one of embodiments 1-314, wherein one R⁵ is —OR¹¹.

346. The compound of any one of embodiments 1-314, wherein a R⁵ is —NR¹¹R¹².

347. The compound of any one of embodiments 1-314, wherein one R⁵ is —NR¹¹R¹².

348. The compound of any one of embodiments 1-314, wherein a R⁵ is —S(O)R¹⁰.

349. The compound of any one of embodiments 1-314, wherein one R⁵ is —S(O)R¹⁰.

350. The compound of any one of embodiments 1-314, wherein a R⁵ is —S(O)₂R¹⁰.

351. The compound of any one of embodiments 1-314, wherein one R⁵ is —S(O)₂R¹⁰.

352. The compound of any one of embodiments 1-314, wherein a R⁵ is —OS(O)R¹⁰.

353. The compound of any one of embodiments 1-314, wherein one R⁵ is —OS(O)R¹⁰.

354. The compound of any one of embodiments 1-314, wherein a R⁵ is —OS(O)₂R¹⁰.

355. The compound of any one of embodiments 1-314, wherein one R⁵ is —OS(O)₂R¹⁰.

356. The compound of any one of embodiments 1-314, wherein a R⁵ is —NR¹¹S(O)R¹⁰.

357. The compound of any one of embodiments 1-314, wherein one R⁵ is —NR¹¹S(O)R¹⁰.

358. The compound of any one of embodiments 1-314, wherein a R⁵ is —NR¹¹S(O)₂R¹⁰.

359. The compound of any one of embodiments 1-314, wherein one R⁵ is —NR¹¹S(O)₂R¹⁰.

360. The compound of any one of embodiments 1-314, wherein a R^{S} is —SR¹¹.

361. The compound of any one of embodiments 1-314, wherein one R⁵ is —SR¹¹.

362. The compound of any one of embodiments 1-362, wherein R¹¹ and R¹² are hydrogen.

363. The compound of any one of embodiments 1-362, wherein a R¹¹ is hydrogen.

364. The compound of any one of embodiments 1-362, wherein a R¹² is hydrogen.

365. The compound of any one of embodiments 1-362, wherein R¹¹ and R¹² are alkyl.

366. The compound of any one of embodiments 1-362, wherein a R¹¹ is alkyl.

367. The compound of any one of embodiments 1-362, wherein a R¹² is alkyl.

368. The compound of any one of embodiments 1-362, wherein R¹¹ and R¹² are methyl.

369. The compound of any one of embodiments 1-362, wherein a R¹¹ is methyl.

370. The compound of any one of embodiments 1-362, wherein a R¹² is methyl.

371. The compound of any one of embodiments 1-362, wherein R¹¹ or R¹² is haloalkyl optionally substituted as allowed by valence with 1, 2, 3, or 4 substituents selected from R³¹.

372. The compound of any one of embodiments 1-362, wherein R¹¹ or R¹² is alkenyl or alkynyl optionally substituted as allowed by valence with 1, 2, 3, or 4 substituents selected from R³¹.

373. The compound of any one of embodiments 1-362, wherein R¹¹ or R¹² is aryl optionally substituted as allowed by valence with 1, 2, 3, or 4 substituents selected from R³¹.

374. The compound of any one of embodiments 1-362, wherein R¹¹ or R¹² is phenyl optionally substituted as allowed by valence with 1, 2, 3, or 4 substituents selected from R³¹.

375. The compound of any one of embodiments 1-362, wherein R¹¹ or R¹² is heterocycle optionally substituted as allowed by valence with 1, 2, 3, or 4 substituents selected from R³¹.

376. The compound of any one of embodiments 1-362, wherein R¹¹ or R¹² is heteroaryl optionally substituted as allowed by valence with 1, 2, 3, or 4 substituents selected from R³¹.

377. The compound of any one of embodiments 1-362, wherein R¹¹ or R¹² is —C(O)R⁴⁰ optionally substituted as allowed by valence with 1, 2, 3, or 4 substituents selected from R³¹.

378. The compound of any one of embodiments 1-362, wherein R¹¹ or R¹² is —S(O)R⁴⁰ optionally substituted as allowed by valence with 1, 2, 3, or 4 substituents selected from R³¹.

379. The compound of any one of embodiments 1-362, wherein R¹¹ or R¹² is —S(O)₂R⁴⁰ optionally substituted as allowed by valence with 1, 2, 3, or 4 substituents selected from R³¹.

380. The compound of any one of embodiments 1-379, wherein R³⁰ or R³¹ is hydrogen.

381. The compound of any one of embodiments 1-379, wherein R³⁰ or R³¹ is halogen.

382. The compound of any one of embodiments 1-379, wherein R³⁰ or R³¹ is alkyl optionally substituted as allowed by valence with 1, 2, 3, or 4 substituents selected from R⁴³.

383. The compound of any one of embodiments 1-379, wherein R³⁰ or R³¹ is haloalkyl optionally substituted as allowed by valence with 1, 2, 3, or 4 substituents selected from R⁴³.

384. The compound of any one of embodiments 1-379, wherein R³⁰ or R³¹ is alkenyl optionally substituted as allowed by valence with 1, 2, 3, or 4 substituents selected from R⁴³.

385. The compound of any one of embodiments 1-379, wherein R³⁰ or R³¹ is alkynyl optionally substituted as allowed by valence with 1, 2, 3, or 4 substituents selected from R⁴³.

386. The compound of any one of embodiments 1-379, wherein R³⁰ or R³¹ is heterocycle optionally substituted as allowed by valence with 1, 2, 3, or 4 substituents selected from R⁴³.

387. The compound of any one of embodiments 1-379, wherein R³⁰ or R³¹ is aryl optionally substituted as allowed by valence with 1, 2, 3, or 4 substituents selected from R⁴³.

388. The compound of any one of embodiments 1-379, wherein R³⁰ or R³¹ is heteroaryl optionally substituted as allowed by valence with 1, 2, 3, or 4 substituents selected from R⁴³.

389. The compound of any one of embodiments 1-379, wherein R³⁰ or R³¹ is cyano.

390. The compound of any one of embodiments 1-379, wherein R³⁰ or R³¹ is nitro.

391. The compound of any one of embodiments 1-379, wherein R³⁰ or R³¹ is —C(O)R⁴⁰.

392. The compound of any one of embodiments 1-379, wherein R³⁰ or R³¹ is —OC(O)R⁴⁰.

393. The compound of any one of embodiments 1-379, wherein R³⁰ or R³¹ is —NR⁴¹C(O)R⁴⁰.

394. The compound of any one of embodiments 1-379, wherein R³⁰ or R³¹ is —OR⁴¹.

395. The compound of any one of embodiments 1-379, wherein R³⁰ or R³¹ is —NR⁴¹R⁴²

396. The compound of any one of embodiments 1-379, wherein R³⁰ or R³¹ is —S(O)R⁴⁰.

397. The compound of any one of embodiments 1-379, wherein R³⁰ or R³¹ is —S(O)₂R⁴⁰.

398. The compound of any one of embodiments 1-379, wherein R³⁰ or R³¹ is —OS(O)R⁴⁰.

399. The compound of any one of embodiments 1-379, wherein R³⁰ or R³¹ is —OS(O)₂R⁴⁰.

400. The compound of any one of embodiments 1-379, wherein R³⁰ or R³¹ is —NR⁴¹S(O)R⁴⁰.

401. The compound of any one of embodiments 1-379, wherein R³⁰ or R³¹ is —NR⁴¹S(O)₂R⁴⁰.

402. The compound of any one of embodiments 1-379, wherein R³⁰ or R³¹ is —SR⁴¹.

403. The compound of any one of embodiments 1-402, wherein Linker is of Formula:

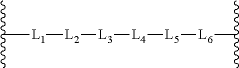

wherein
L₁, L₂, L₃, L₄, L₅, and L₆ are independently selected from the group consisting of a bond, alkyl, alkene, alkyne, haloalkyl, alkoxy, aryl, heterocycle, heteroaryl, bicycle, —C(O)—, —C(O)O—, —OC(O)—, —SO₂—, —S(O)—, —C(S)—, —C(O)NR¹¹—, —NR¹¹C(O)—, —O—, —S—, —NR¹¹—, —P(O)(OR¹¹)O—, —P(O)(OR¹¹)—, polyethylene glycol, lactic acid, and glycolic acid, each of which except bond is optionally substituted with 1, 2, 3, or 4 substituents independently selected from R⁴⁴; wherein L₁, L₂, L₃, L₄, L₅, and L₆ are selected such that there are no more than two of the same moieties connected together (e.g, L₁, L₂, and L₃ cannot all three be —C(O)—) and O and N atoms are not directly linked together except within aromatic rings (e.g. L₁ and L₂ cannot both be —O— or NR¹¹);
R⁴⁴ is independently selected at each instance from hydrogen, alkyl, haloalkyl, alkenyl, alkynyl, aryl, heterocycle, heteroaryl, amino, hydroxyl, alkoxy, —NR¹¹R¹², halogen, cyano, nitro, —OC(O)R⁴⁰, —NR¹¹C(O)R⁴⁰, —C(O)R⁴⁰, —OP(O)(R⁴⁰)₂, —P(O)(R⁴⁰)₂, —NR¹¹P(O)(R⁴⁰)₂, —SR¹¹, —OR¹¹, —S(O)R⁴⁰, —S(O)₂R⁴⁰, and —N(alkyl)C(O)R⁴⁰, each of which except hydrogen is optionally substituted as allowed by valence with 1, 2, 3, or 4 substituents selected from R⁴⁵;
R⁴⁵ is independently selected at each instance from hydrogen, halogen, cyano, nitro, alkyl, haloalkyl, alkenyl, alkynyl, aryl, heterocycle, heteroaryl, amino, hydroxyl, alkoxy, —NHalkyl, —N(alkyl)₂, —OC(O)alkyl, —NHC(O)alkyl, and —N(alkyl)C(O)alkyl; and
Linker replaces or is covalently attached to a R¹, R², R³, R^{4a}, R^{4b}, R^{5a}, R^{5b}, R⁷, R⁸, R¹⁰, R¹¹, or R¹².

404. The compound of embodiment 403, wherein Linker-Ubiquitinated Protein Targeting Ligand replaces a R¹, R², R³, R^{4a}, R^{4b}, R^{5a}, R^{5b}, R⁷, R⁸, R¹⁰, R¹¹, or R¹².

405. The compound of embodiment 403, wherein Linker-Ubiquitinated Protein Targeting Ligand is covalently attached to a R¹, R², R³, R^{4a}, R^{4b}, R^{5a}, R^{5b}, R⁷, R⁸, R¹⁰, R¹¹, or R¹² as allowed by valence.

406. The compound of embodiments 403-405, wherein Linker replaces a R¹.

407. The compound of embodiments 403-405, wherein Linker replaces a R².

408. The compound of embodiments 403-405, wherein Linker replaces a R³.

409. The compound of embodiments 403-405, wherein Linker replaces a R^{4a} or R^{4b}.

410. The compound of embodiments 403-405, wherein Linker replaces a R^{5a} or R^{5b}.

411. The compound of embodiments 403-405, wherein Linker replaces a R⁶.

412. The compound of embodiments 403-405, wherein Linker replaces a R⁷.

413. The compound of embodiments 403-405, wherein Linker replaces a R⁸.

414. The compound of embodiments 403-405, wherein Linker replaces a R⁹.

415. The compound of embodiments 403-405, wherein Linker replaces a R¹⁰.

416. The compound of embodiments 403-405, wherein Linker replaces a R¹¹.

417. The compound of embodiments 403-405, wherein Linker replaces a R¹².

418. The compound of embodiments 403-405, wherein Linker is attached to a R¹.

419. The compound of embodiments 403-405, wherein Linker is attached to a R².

420. The compound of embodiments 403-405, wherein Linker is attached to a R³.

421. The compound of embodiments 403-405, wherein Linker is attached to a R^{4a} or R^{4b}.

422. The compound of embodiments 403-405, wherein Linker is attached to a Rsa or R.

423. The compound of embodiments 403-405, wherein Linker is attached to a R⁶.

424. The compound of embodiments 403-405, wherein Linker is attached to a R⁷.

425. The compound of embodiments 403-405, wherein Linker is attached to a R⁸.

426. The compound of embodiments 403-405, wherein Linker is attached to a R⁹.

427. The compound of embodiments 403-405, wherein Linker is attached to a R¹⁰.

428. The compound of embodiments 403-405, wherein Linker is attached to a R¹¹.

429. The compound of embodiments 403-405, wherein Linker is attached to a R¹².

430. The compound of any one of embodiments 1-429, wherein the Ubiquitinated Protein Targeting Ligand is a ligand that binds CFTR.

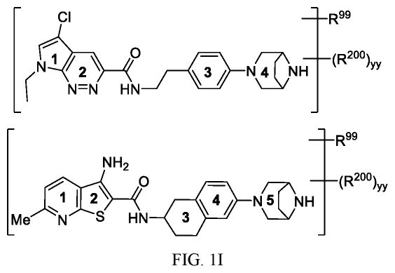

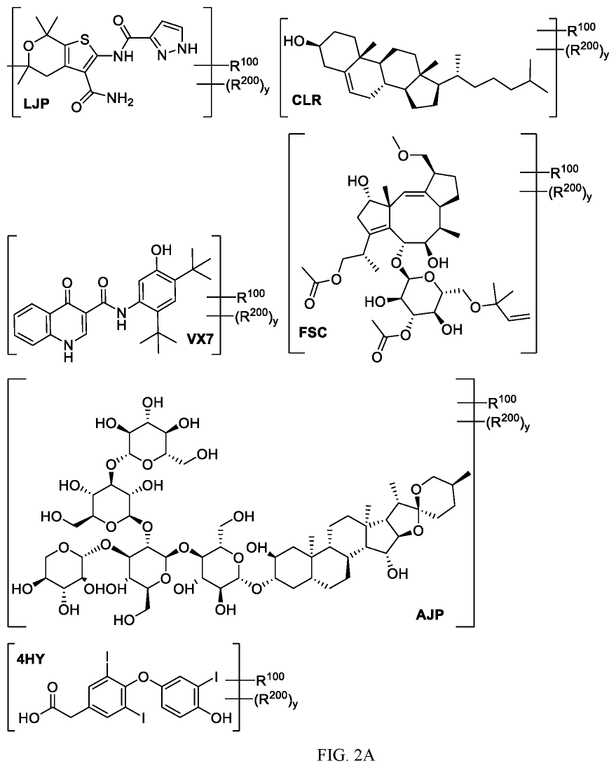

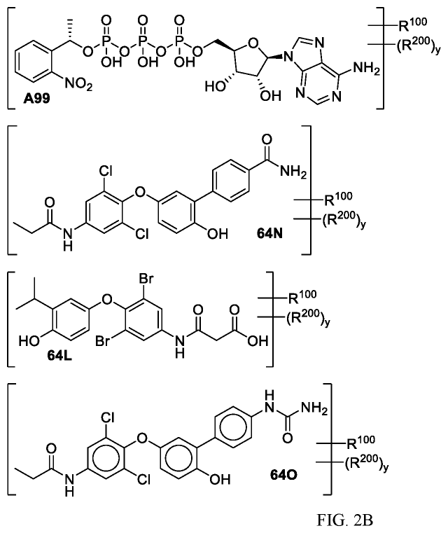

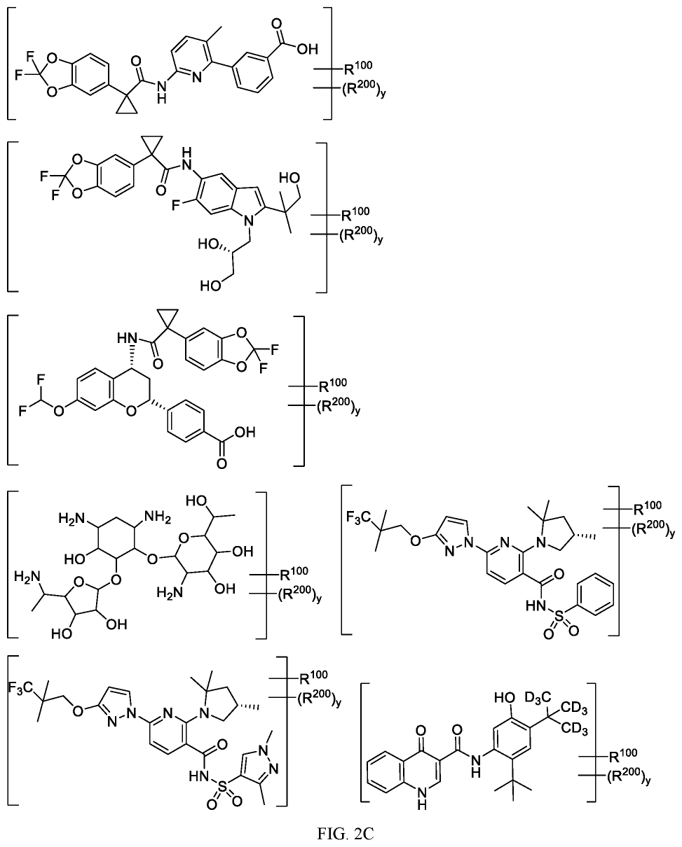

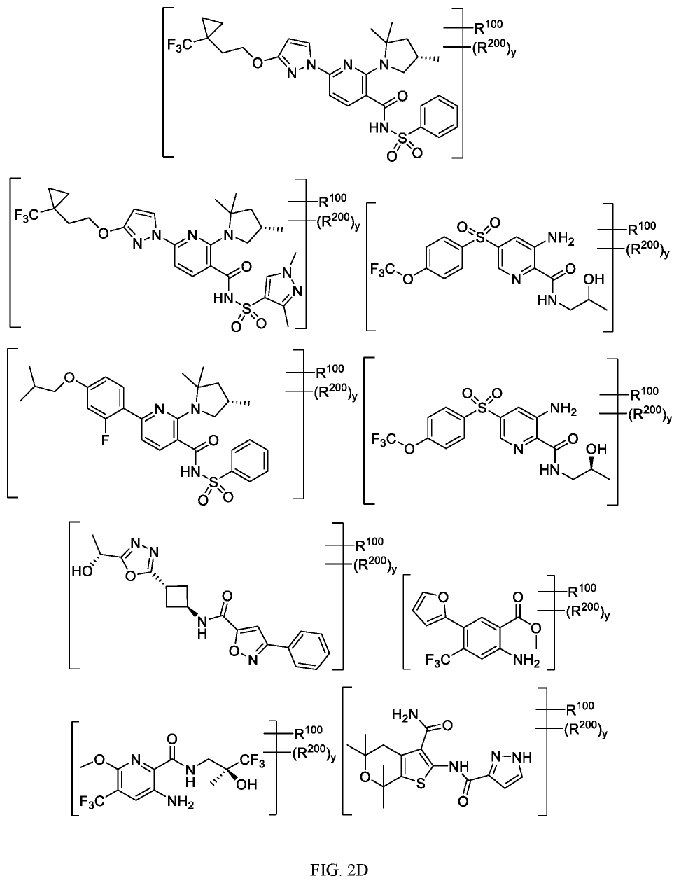

431. The compound of embodiment 430, wherein the Ubiquitinated Protein Targeting Ligand is selected from FIG. 2A, FIG. 2B, FIG. 2C, and FIG. 2D.

432. The compound of any one of embodiments 1-429, wherein the Ubiquitinated Protein Targeting Ligand is a ligand that binds phenylalanine hydroxylase.

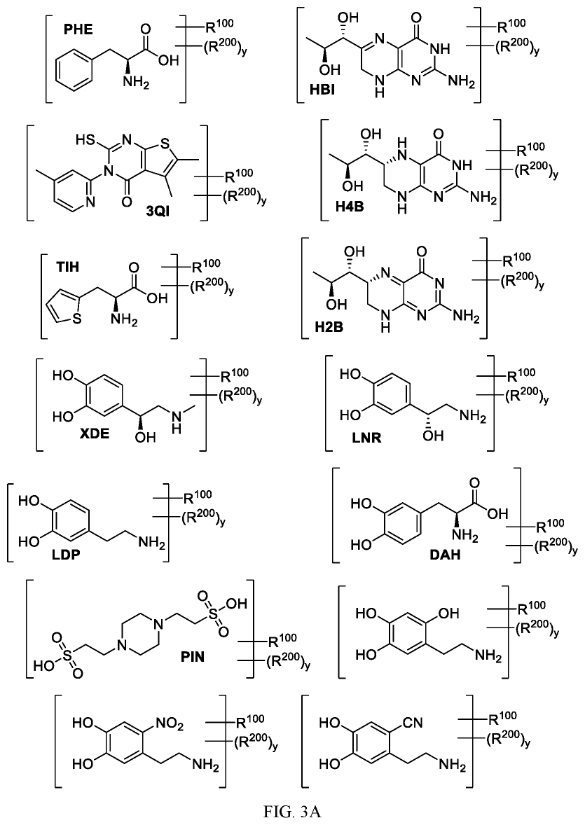

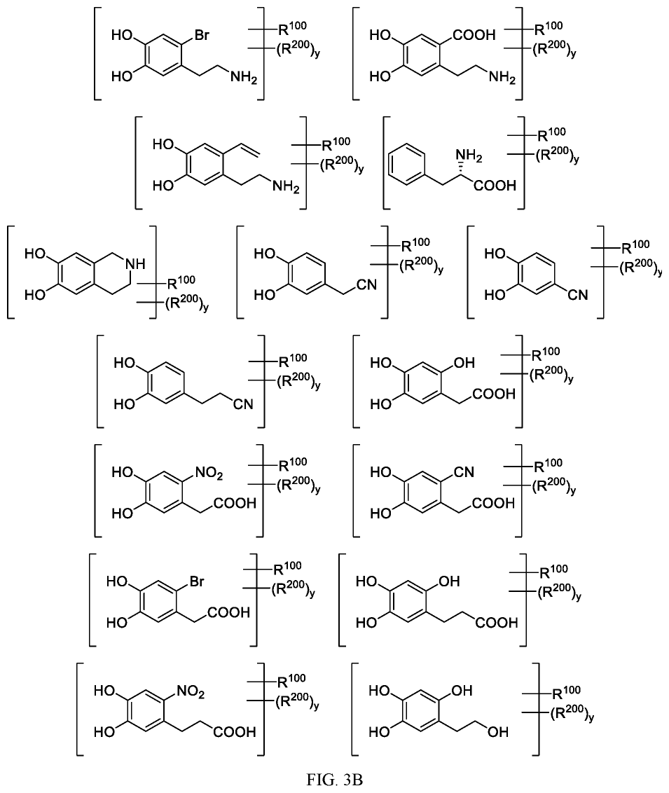

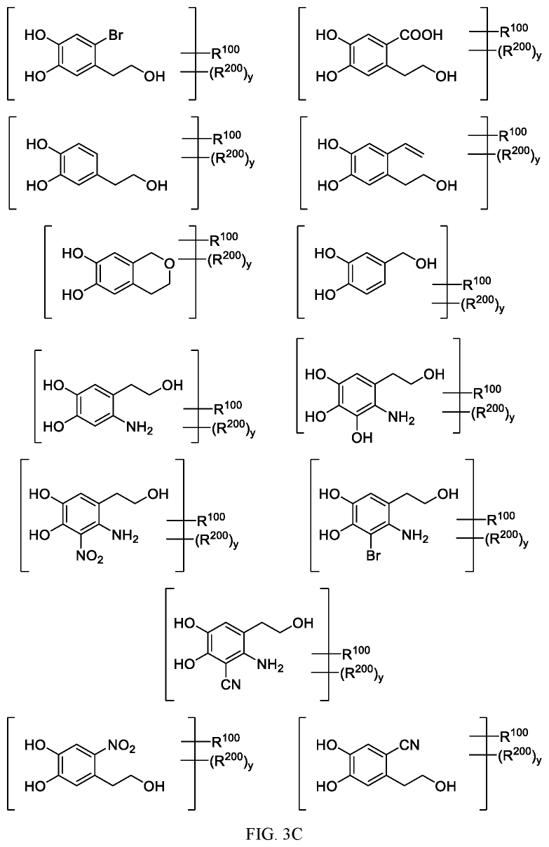

433. The compound of embodiment 432, wherein the Ubiquitinated Protein Targeting Ligand is selected from FIG. 3A, FIG. 3B, and FIG. 3C.

434. The compound of any one of embodiments 1-429, wherein the Ubiquitinated Protein Targeting Ligand is a ligand that binds p53.

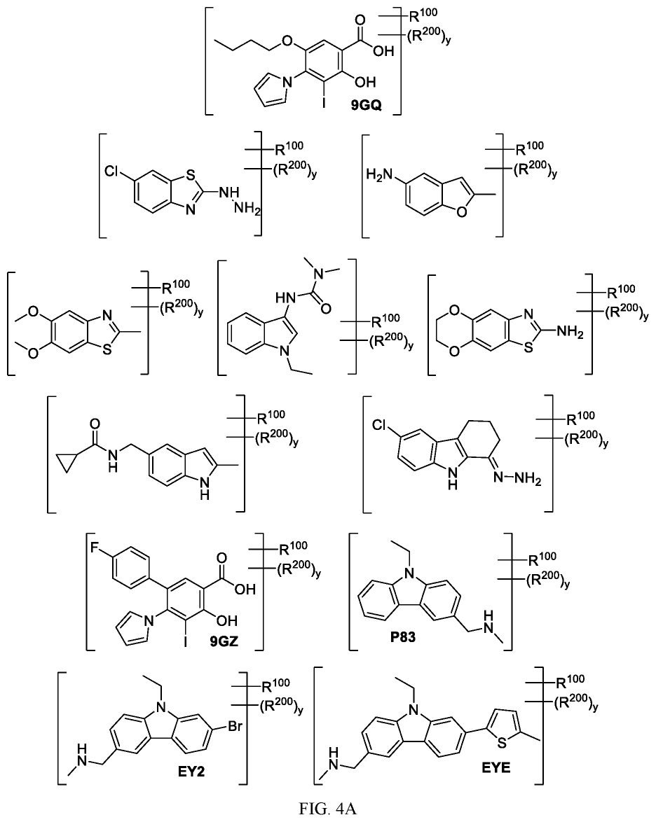

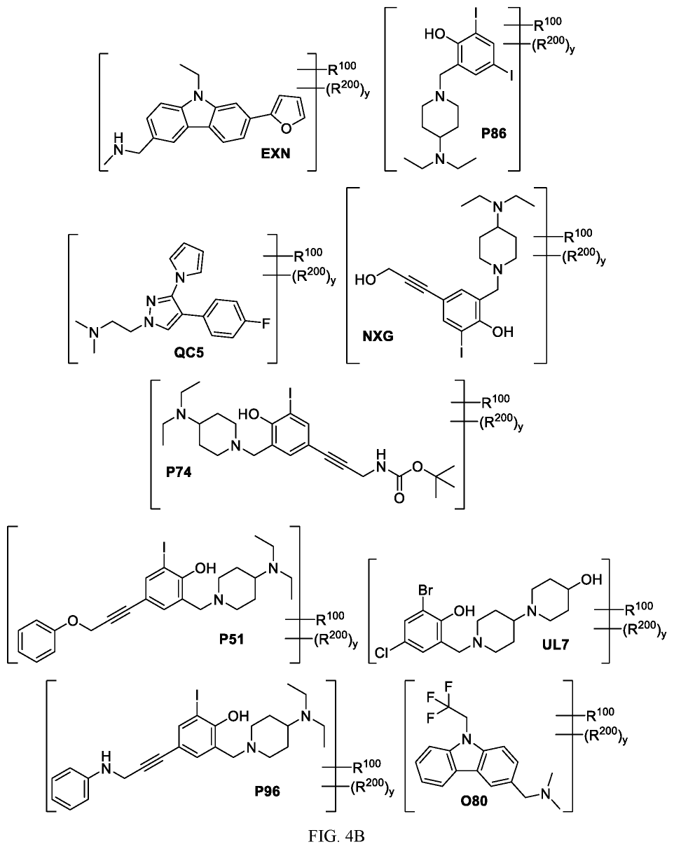

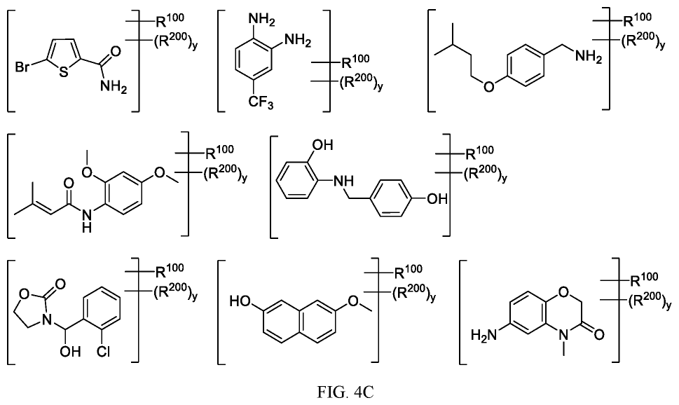

435. The compound of embodiment 434, wherein the Ubiquitinated Protein Targeting Ligand is selected from FIG. 4A, FIG. 4B, and FIG. 4C.

436. The compound of any one of embodiments 1-429, wherein the Ubiquitinated Protein Targeting Ligand is a ligand that binds rhodopsin.

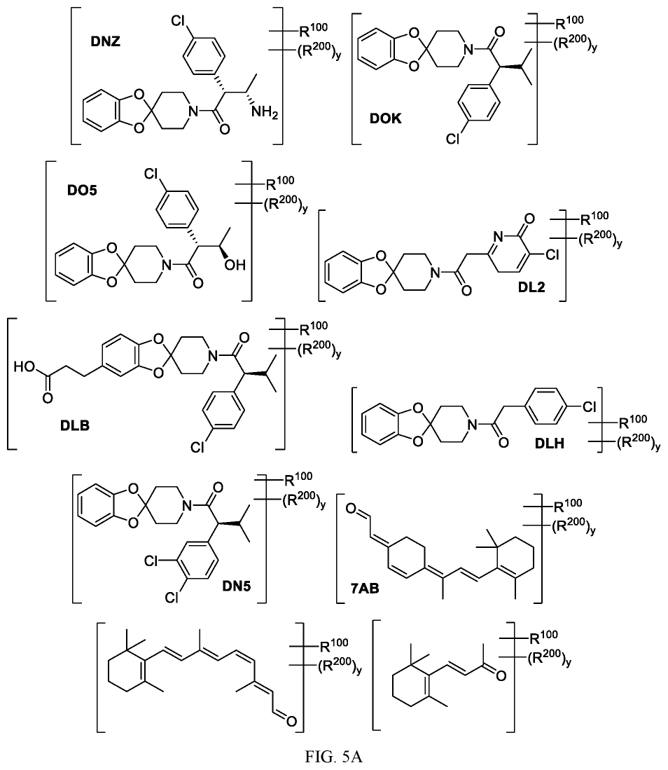

437. The compound of embodiment 436, wherein the Ubiquitinated Protein Targeting Ligand is selected from FIG. 5A and FIG. 5B.

438. The compound of any one of embodiments 1-429, wherein the Ubiquitinated Protein Targeting Ligand is a ligand that binds c-myc.

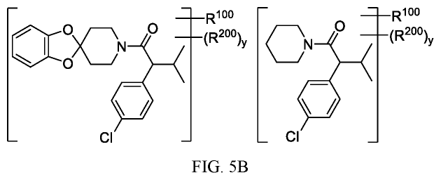

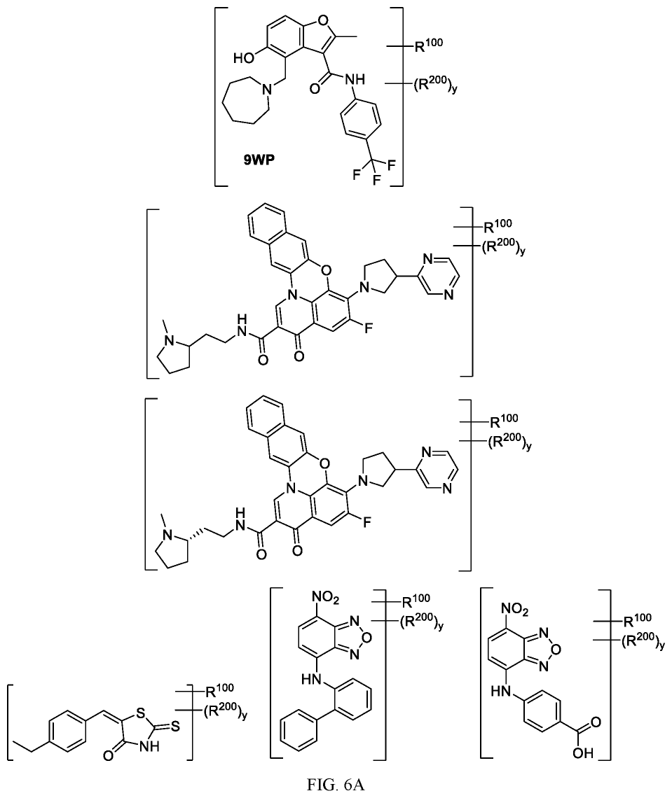

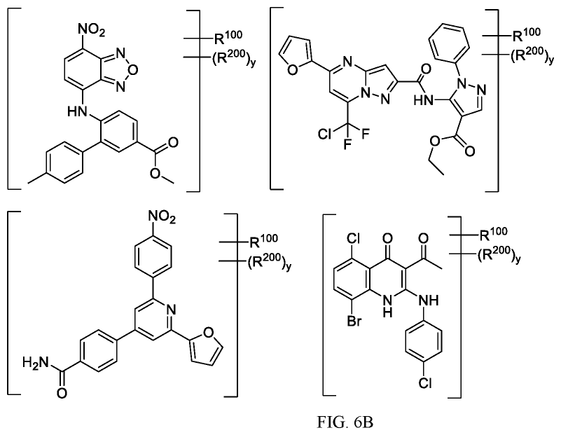

439. The compound of embodiment 438, wherein the Ubiquitinated Protein Targeting Ligand is selected from FIG. 6A and FIG. 6B.

440. The compound of any one of embodiments 1-429, wherein the Ubiquitinated Protein Targeting Ligand is a ligand that binds RIPK1.

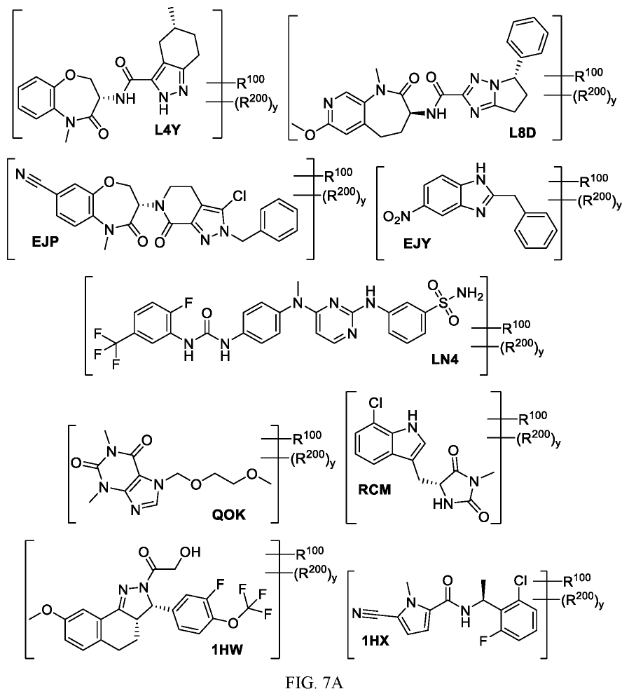

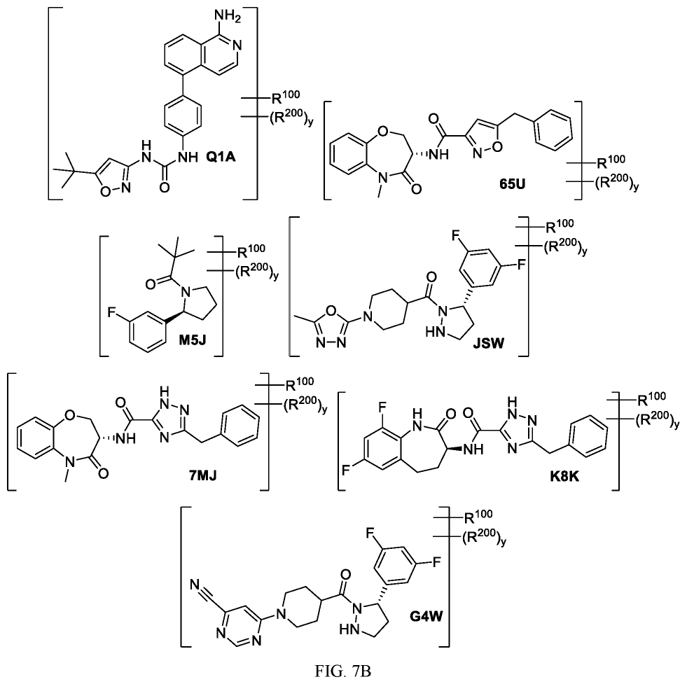

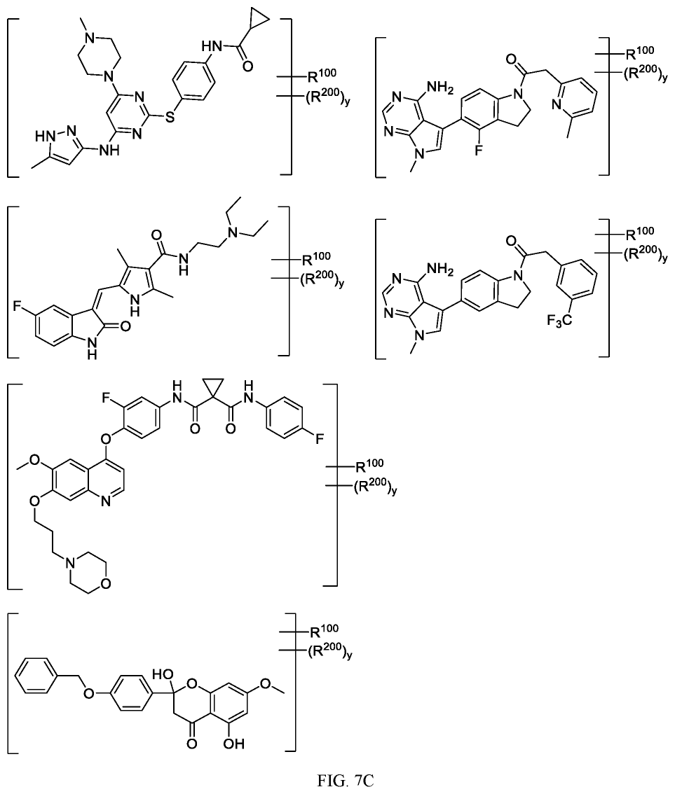

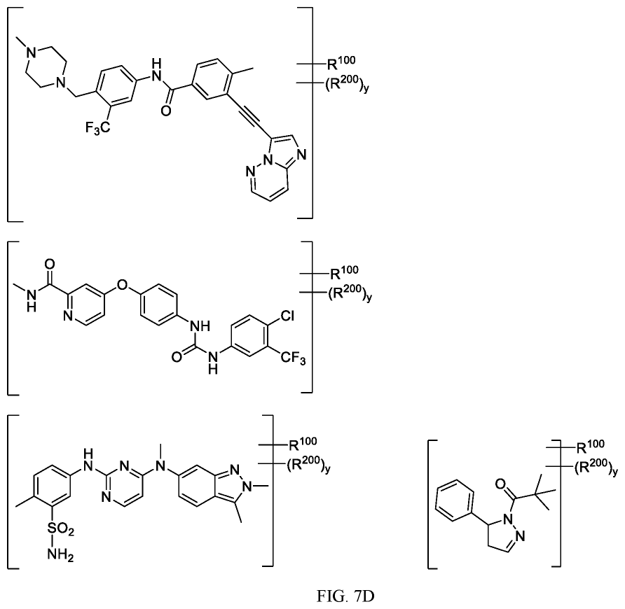

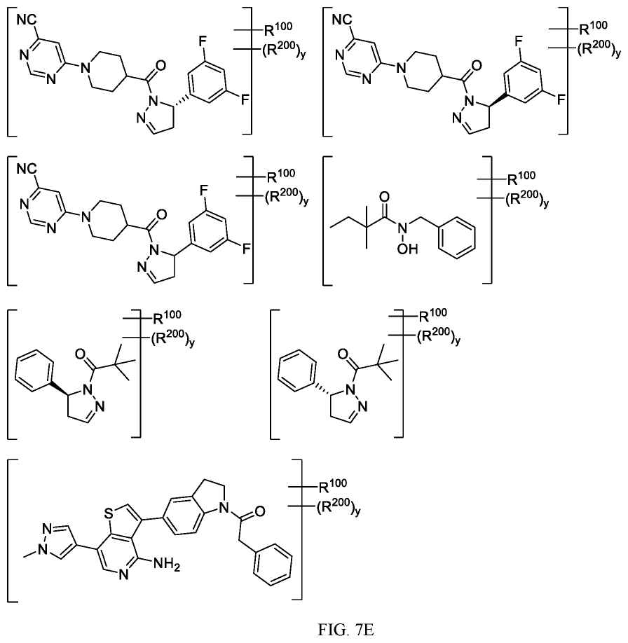

441. The compound of embodiment 440, wherein the Ubiquitinated Protein Targeting Ligand is selected from FIG. 7A, FIG. 7B, FIG. 7C, FIG. 7D, and FIG. 7E.

442. The compound of any one of embodiments 1-429, wherein the Ubiquitinated Protein Targeting Ligand is a ligand that binds RIPK1.

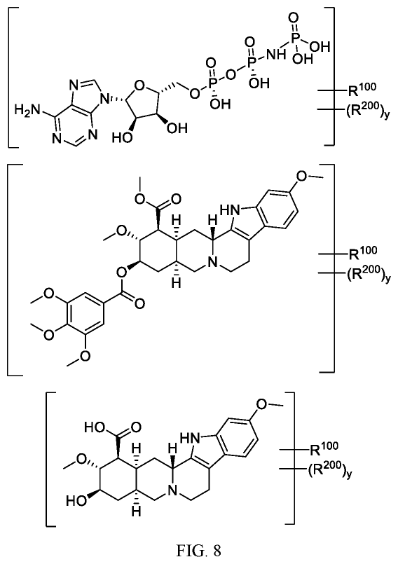

443. The compound of embodiment 442, wherein the Ubiquitinated Protein Targeting Ligand is selected from FIG. 8.

444. The compound of any one of embodiments 1-429, wherein the Ubiquitinated Protein Targeting Ligand is a ligand that binds CDKN1B.

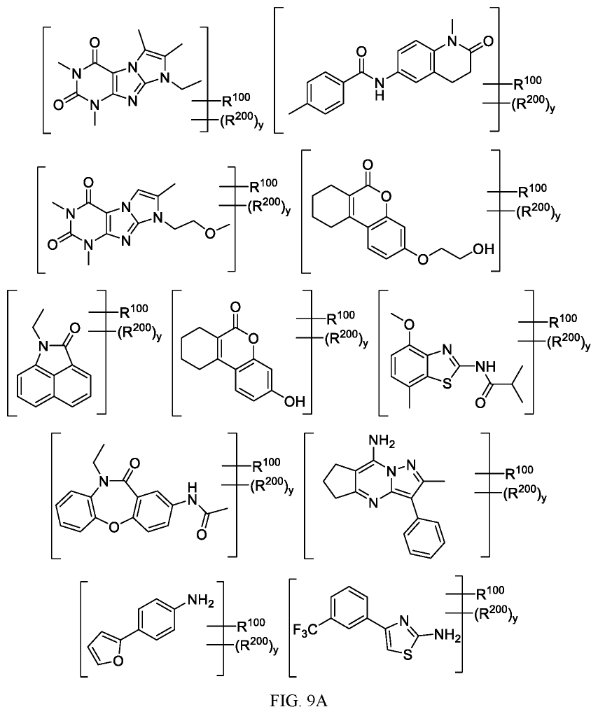

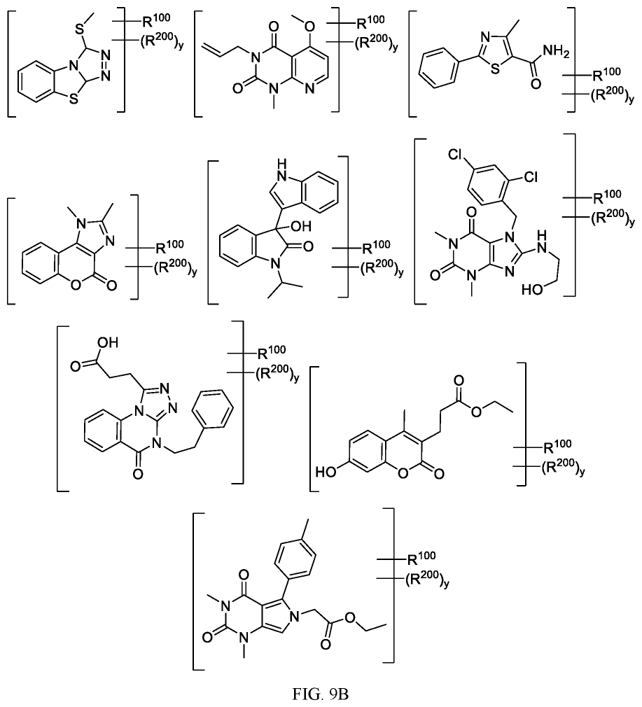

445. The compound of embodiment 444, wherein the Ubiquitinated Protein Targeting Ligand is selected from FIG. 9A and FIG. 9B.

446. The compound of any one of embodiments 1-429, wherein the Ubiquitinated Protein Targeting Ligand is a ligand that binds ABCA4.

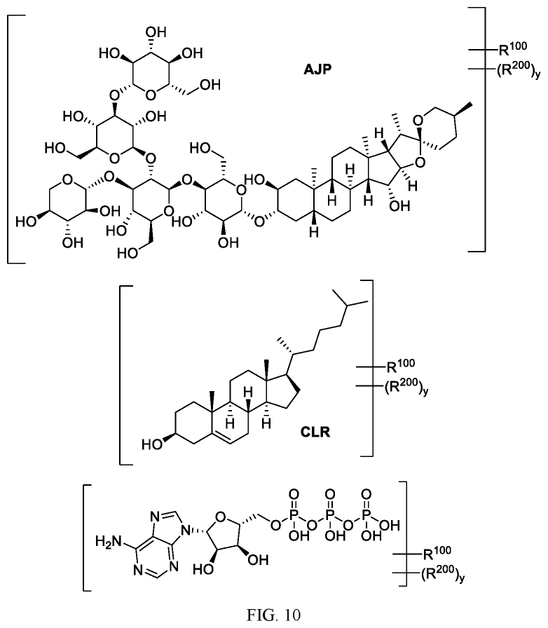

447. The compound of embodiment 446, wherein the Ubiquitinated Protein Targeting Ligand is selected from FIG. 10.

448. The compound of any one of embodiments 1-429, wherein the Ubiquitinated Protein Targeting Ligand is a ligand that binds ABCB11.

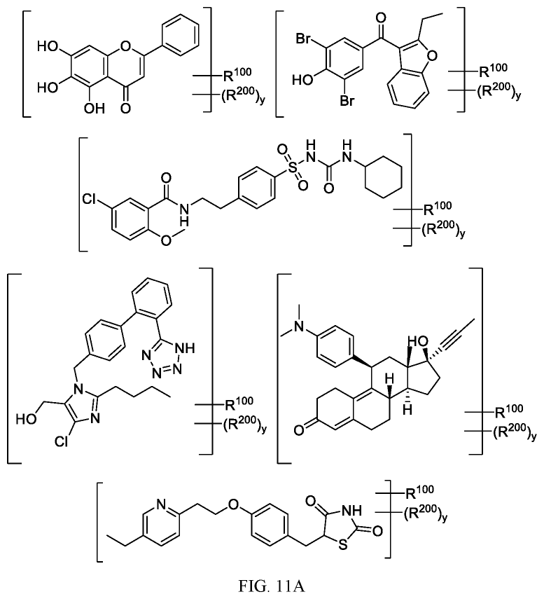

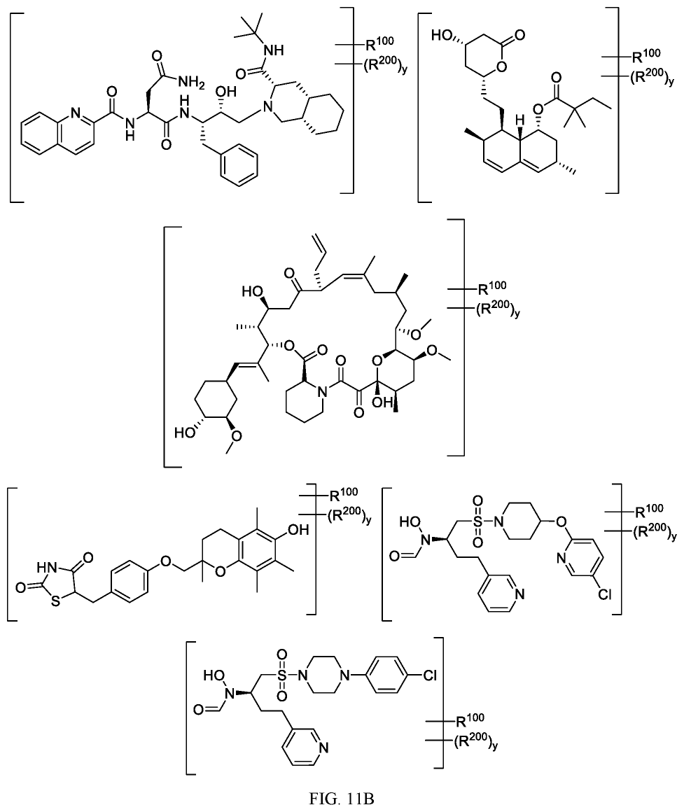

449. The compound of embodiment 448, wherein the Ubiquitinated Protein Targeting Ligand is selected from FIG. 11A and FIG. 11B.

450. The compound of any one of embodiments 1-429, wherein the Ubiquitinated Protein Targeting Ligand is a ligand that binds choline acetylase.

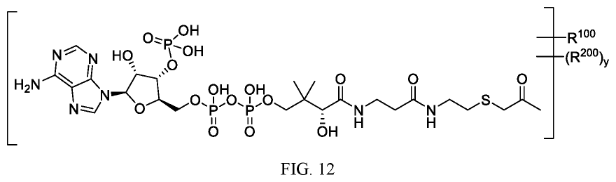

451. The compound of embodiment 450, wherein the Ubiquitinated Protein Targeting Ligand is selected from FIG. 12.

452. The compound of any one of embodiments 1-429, wherein the Ubiquitinated Protein Targeting Ligand is a ligand that binds CYLD.

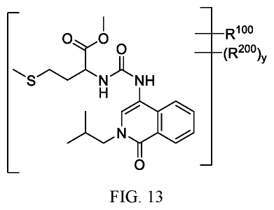

453. The compound of embodiment 452, wherein the Ubiquitinated Protein Targeting Ligand is selected from FIG. 13.

454. The compound of any one of embodiments 1-429, wherein the Ubiquitinated Protein Targeting Ligand is a ligand that binds NEMO.

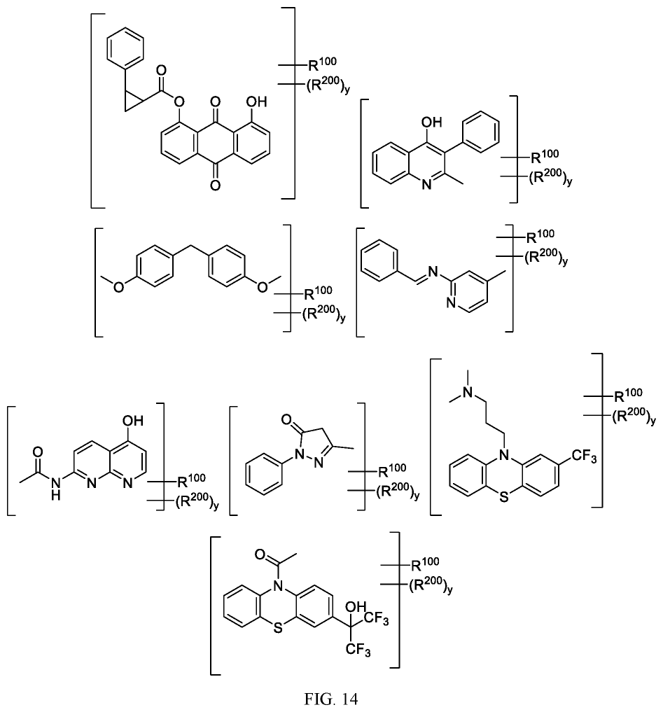

455. The compound of embodiment 454, wherein the Ubiquitinated Protein Targeting Ligand is selected from FIG. 14.

456. The compound of any one of embodiments 1-429, wherein the Ubiquitinated Protein Targeting Ligand is a ligand that binds AH receptor-interacting protein.

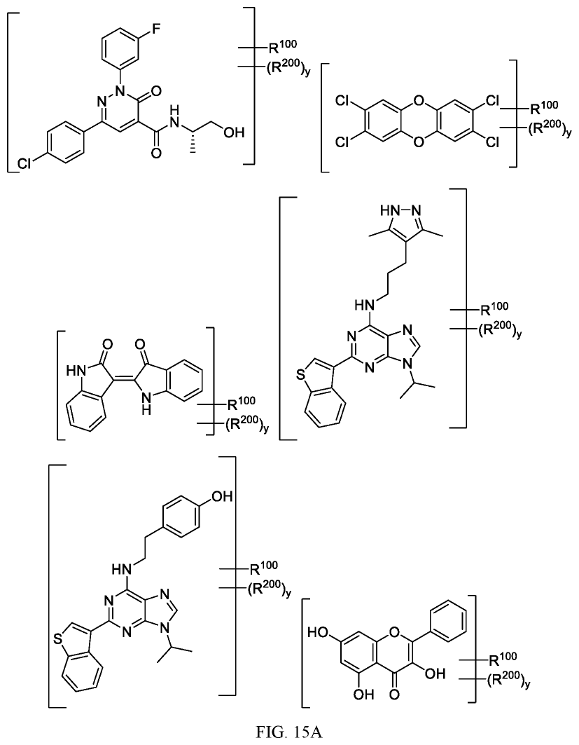

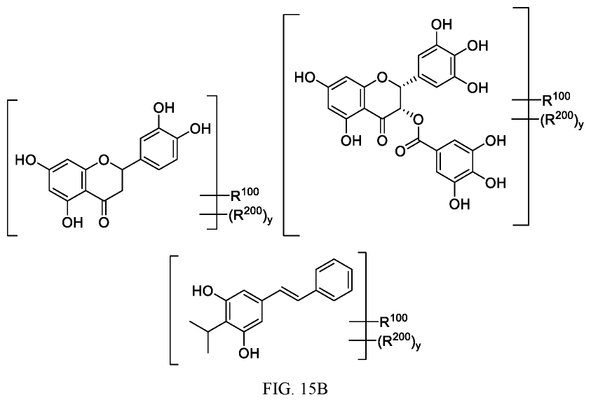

457. The compound of embodiment 456, wherein the Ubiquitinated Protein Targeting Ligand is selected from FIG. 15A and FIG. 15B.

458. The compound of any one of embodiments 1-429, wherein the Ubiquitinated Protein Targeting Ligand is a ligand that binds PDCD4.

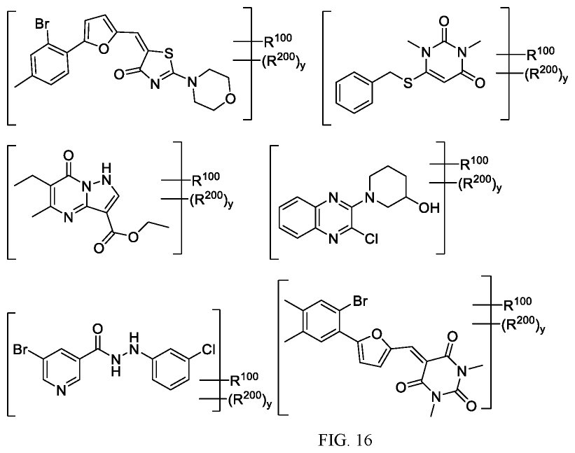

459. The compound of embodiment 458, wherein the Ubiquitinated Protein Targeting Ligand is selected from FIG. 16.

460. The compound of any one of embodiments 1-429, wherein the Ubiquitinated Protein Targeting Ligand is a ligand that binds RIPK2.

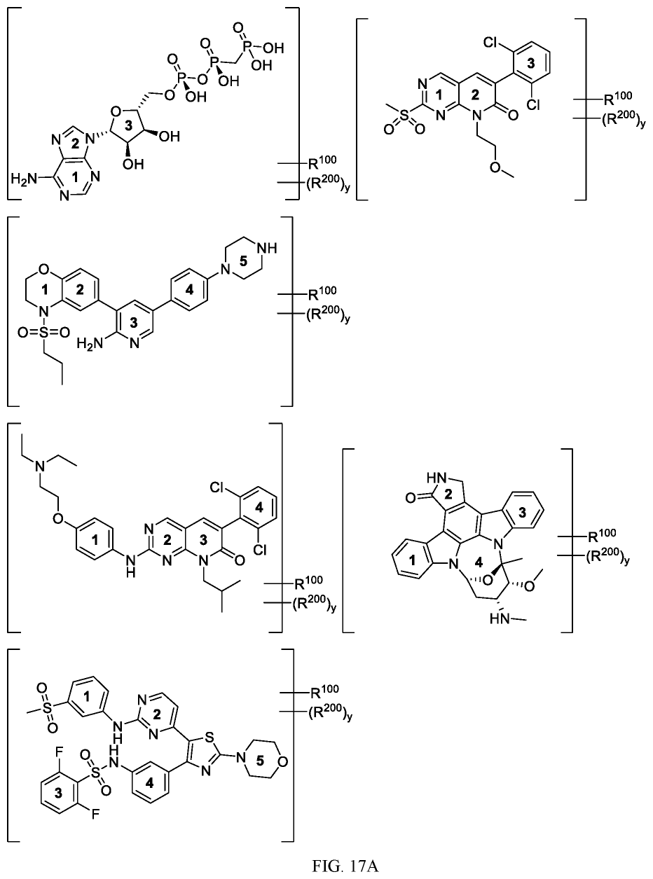

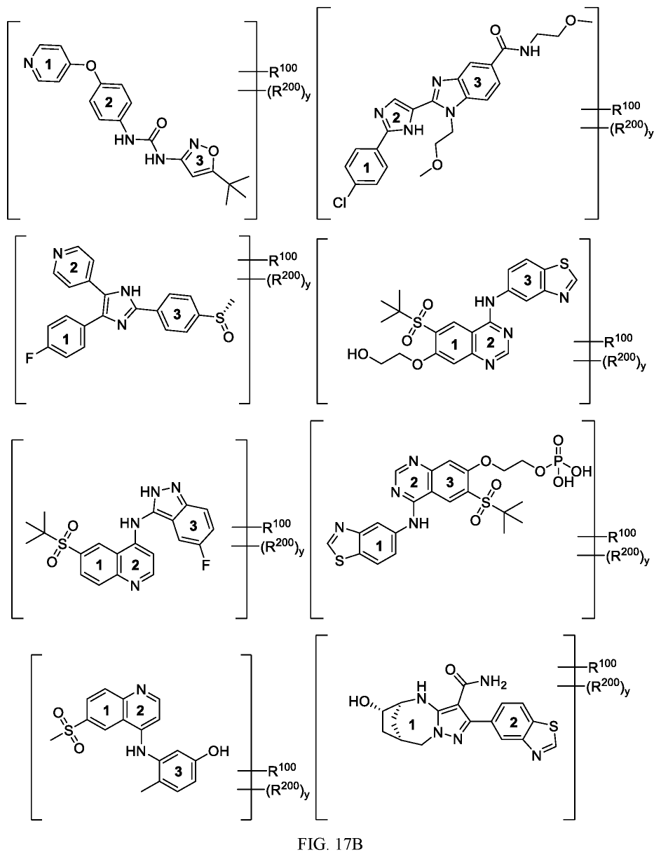

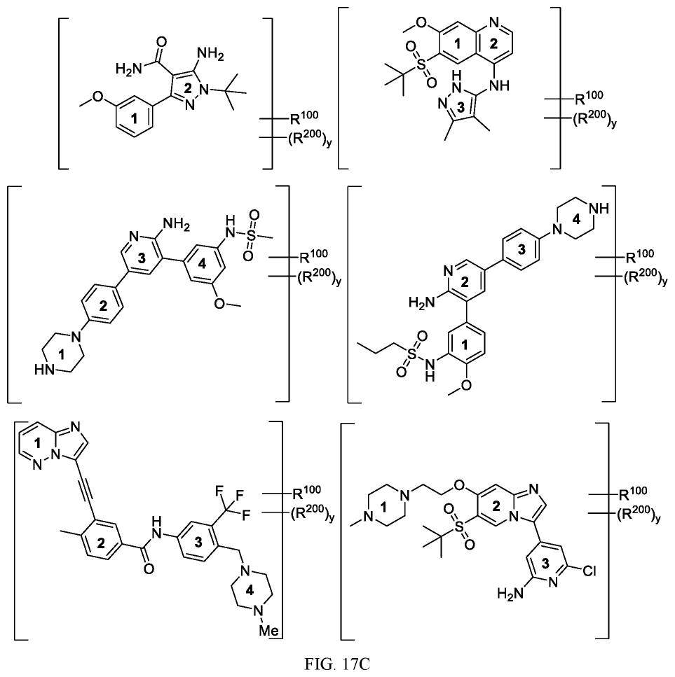

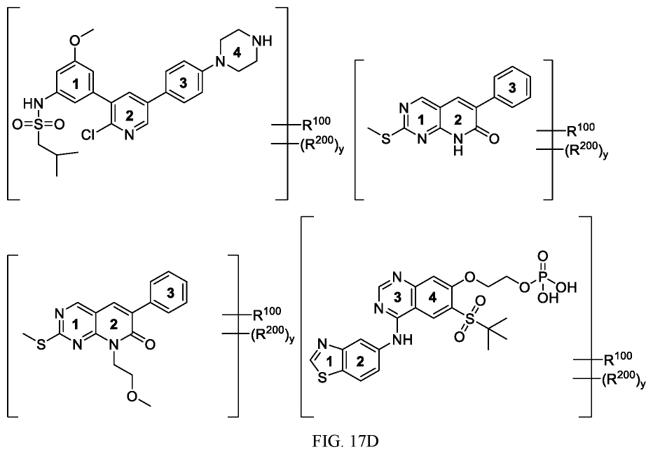

461. The compound of embodiment 460, wherein the Ubiquitinated Protein Targeting Ligand is selected from FIG. 17A, FIG. 17B, FIG. 17C, and FIG. 17D.

462. The compound of any one of embodiments 1-429, wherein the Ubiquitinated Protein Targeting Ligand is a ligand that binds BAX.

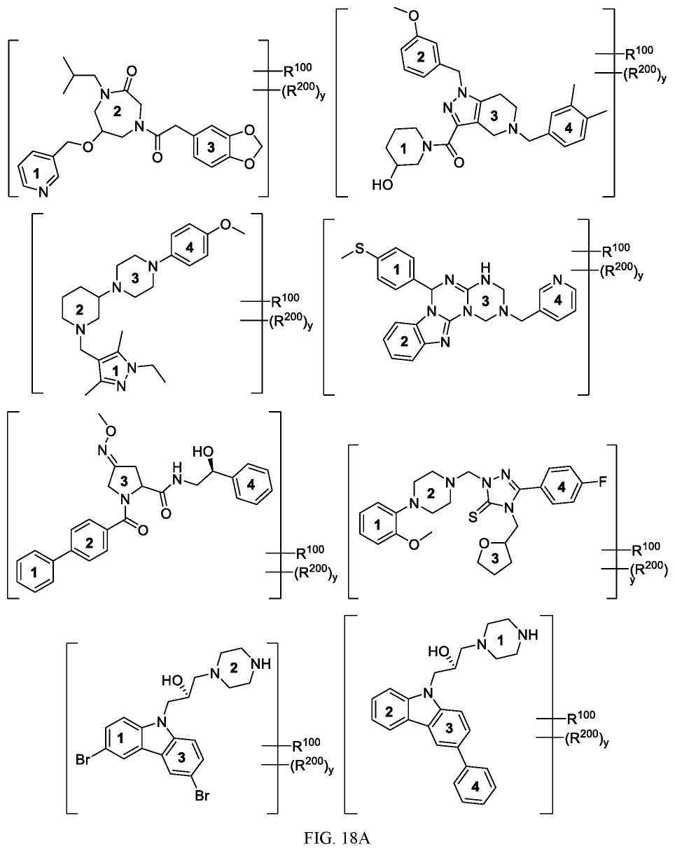

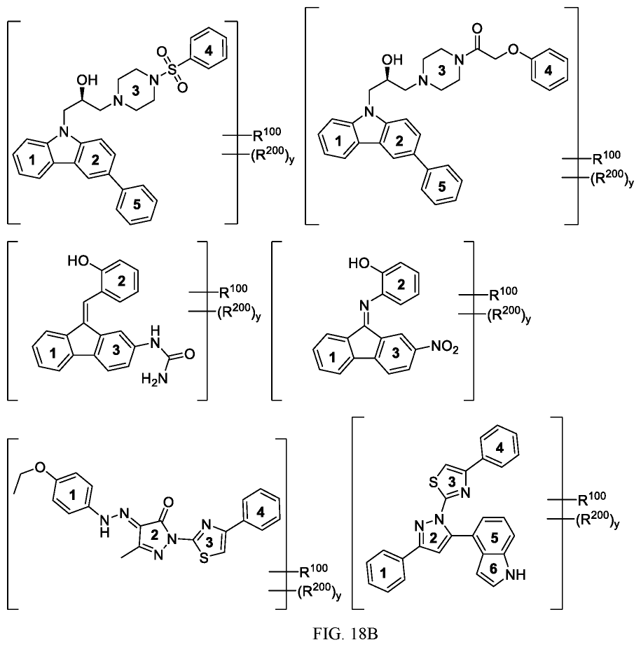

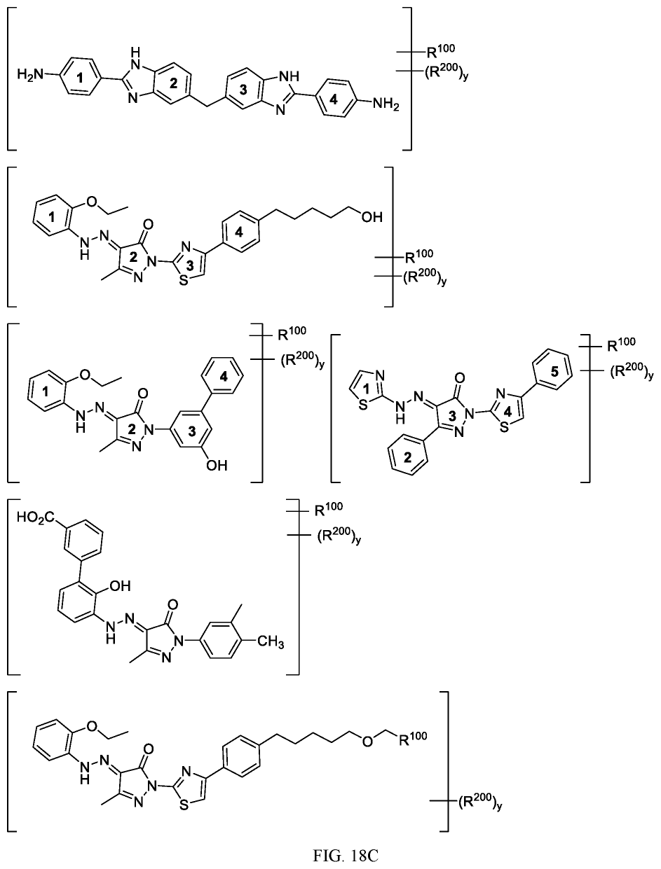

463. The compound of embodiment 462, wherein the Ubiquitinated Protein Targeting Ligand is selected from FIG. 18A, FIG. 18B, and FIG. 18C.

464. The compound of any one of embodiments 1-429, wherein the Ubiquitinated Protein Targeting Ligand is a ligand that binds P21.

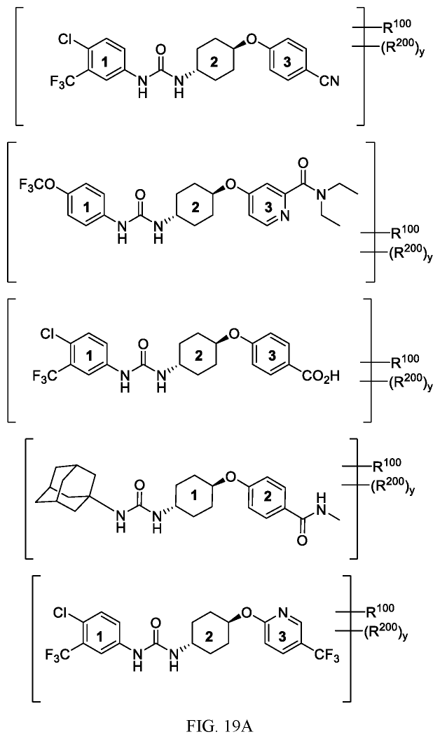

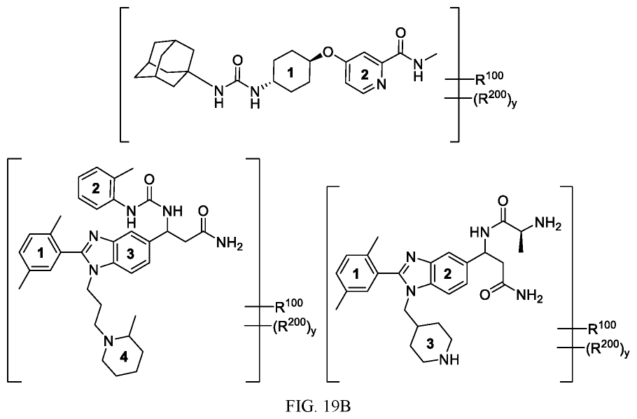

465. The compound of embodiment 464, wherein the Ubiquitinated Protein Targeting Ligand is selected from FIG. 19A and FIG. 19B.

466. The compound of any one of embodiments 1-429, wherein the Ubiquitinated Protein Targeting Ligand is a ligand that binds SERPINA1.

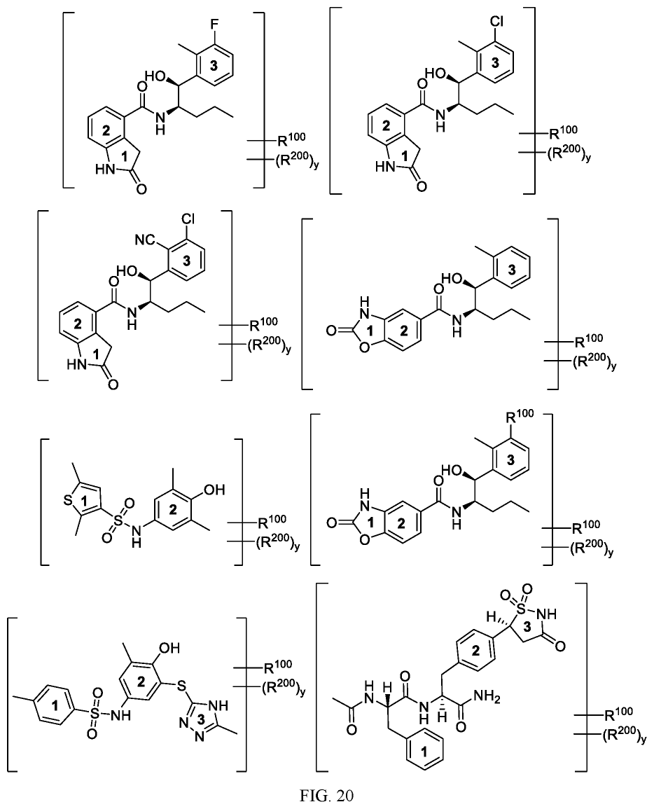

467. The compound of embodiment 466, wherein the Ubiquitinated Protein Targeting Ligand is selected from FIG. 20.

468. The compound of any one of embodiments 1-429, wherein the Ubiquitinated Protein Targeting Ligand is a ligand that binds PKLR.

469. The compound of embodiment 468, wherein the Ubiquitinated Protein Targeting Ligand is selected from FIG. 21A, FIG. 21B, and FIG. 21C.

470. The compound of any one of embodiments 1-429, wherein the Ubiquitinated Protein Targeting Ligand is a ligand that binds KEAPl.

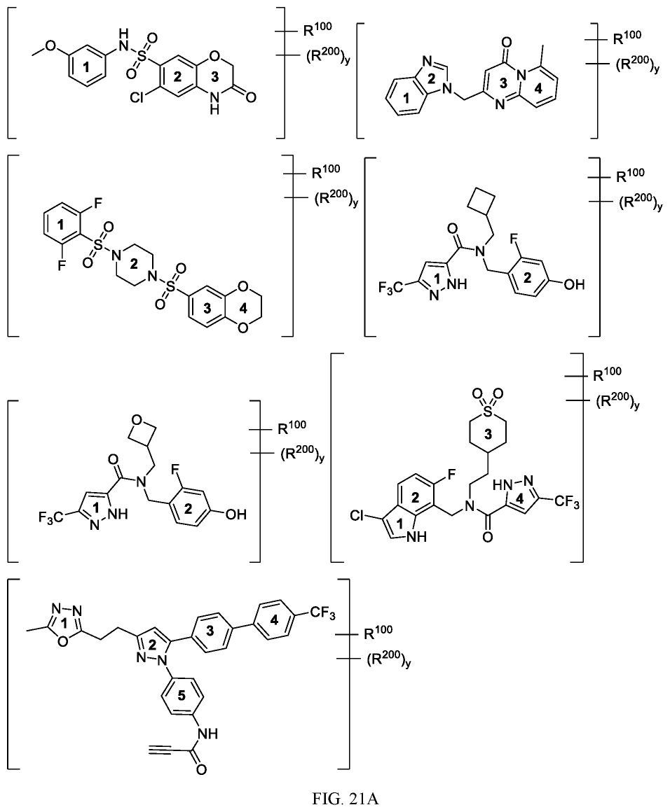

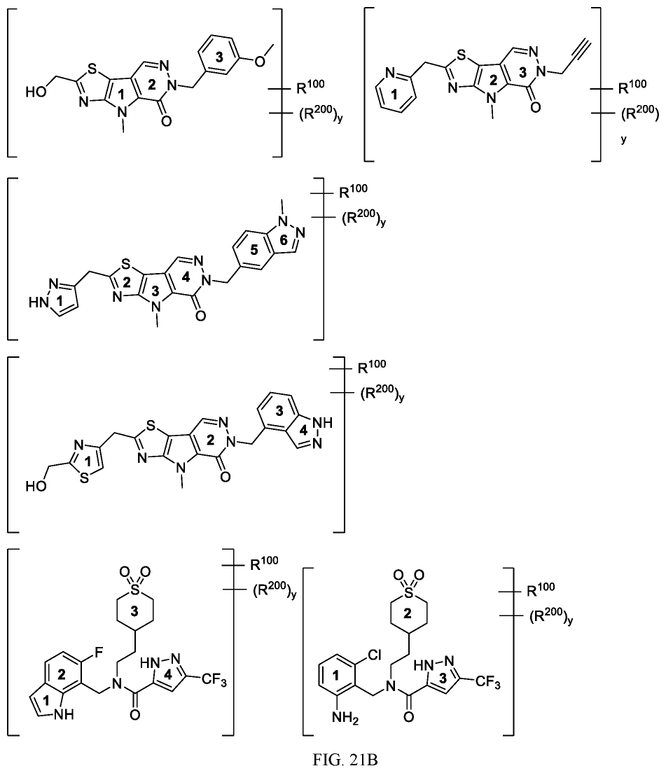

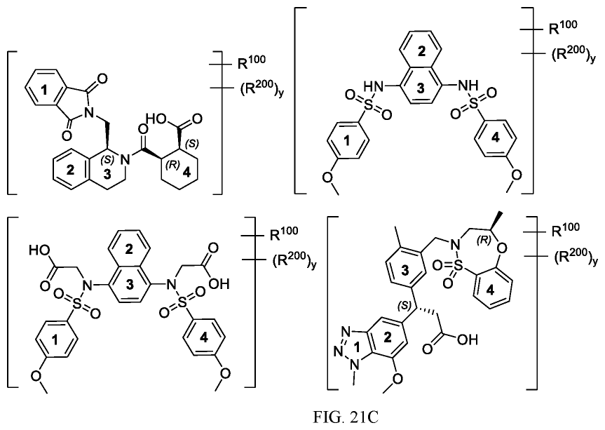

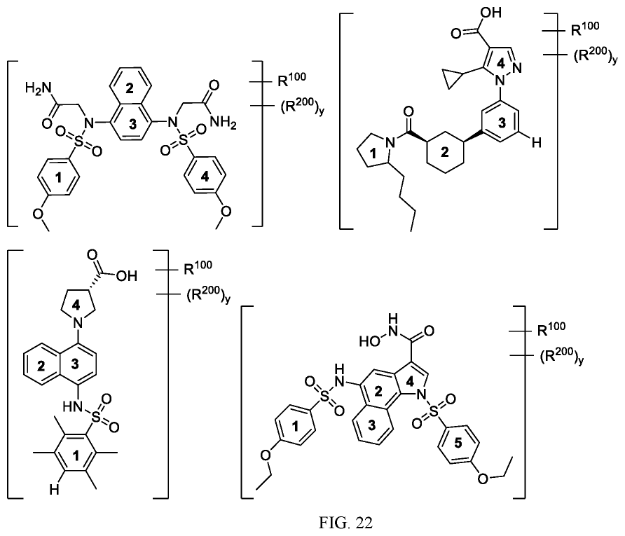

471. The compound of embodiment 470, wherein the Ubiquitinated Protein Targeting Ligand is selected from FIG. 22.

472. The compound of any one of embodiments 1-429, wherein the Ubiquitinated Protein Targeting Ligand is a ligand that binds PTEN.

473. The compound of embodiment 472, wherein the Ubiquitinated Protein Targeting Ligand is selected from FIG. 23.

474. The compound of any one of embodiments 1-429, wherein the Ubiquitinated Protein Targeting Ligand is a ligand that binds IRAK4.

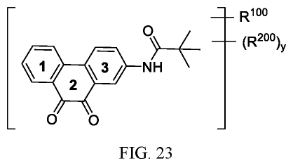

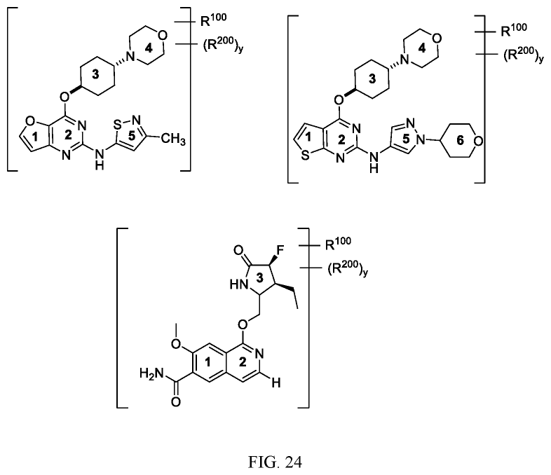

475. The compound of embodiment 474, wherein the Ubiquitinated Protein Targeting Ligand is selected from FIG. 24.

476. The compound of any one of embodiments 1-429, wherein the Ubiquitinated Protein Targeting Ligand is a ligand that binds TK2.

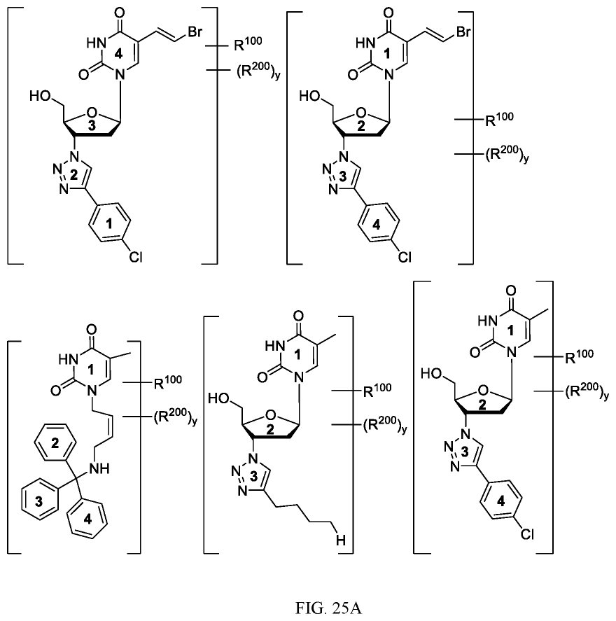

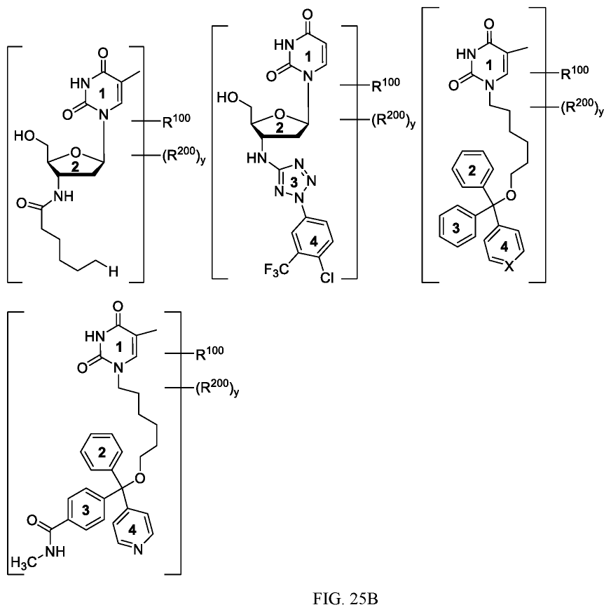

477. The compound of embodiment 476, wherein the Ubiquitinated Protein Targeting Ligand is selected from FIG. 25A and FIG. 25B.

478. The compound of any one of embodiments 1-429, wherein the Ubiquitinated Protein Targeting Ligand is a ligand that binds KCNQ1.

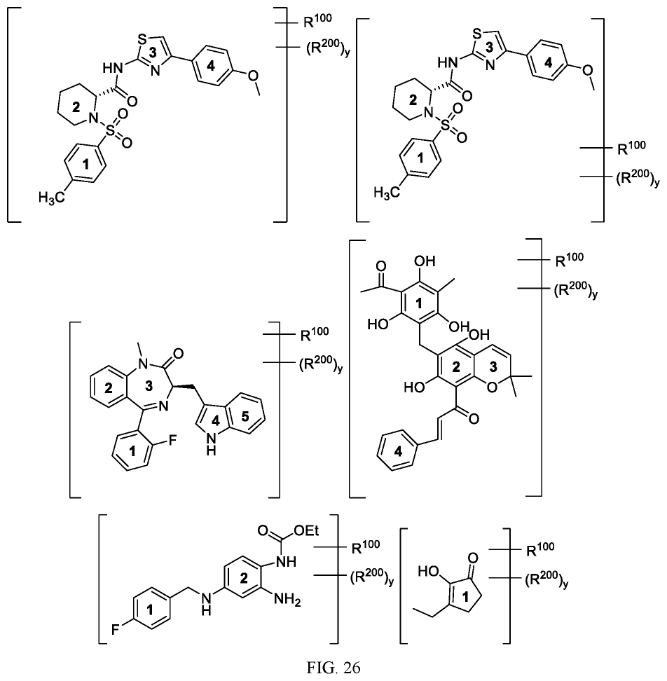

479. The compound of embodiment 478, wherein the Ubiquitinated Protein Targeting Ligand is selected from FIG. 26.

480. The compound of any one of embodiments 1-429, wherein the Ubiquitinated Protein Targeting Ligand is a ligand that binds STINGI.

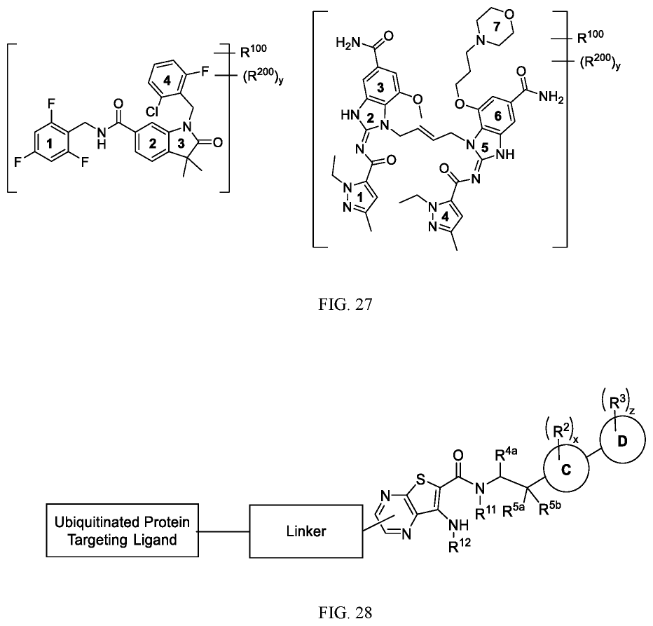

481. The compound of embodiment 480, wherein the Ubiquitinated Protein Targeting Ligand is selected from FIG. 27.

482. A pharmaceutical composition comprising a compound of any one of embodiments 1-481, or a pharmaceutically acceptable salt thereof, optionally in a pharmaceutically acceptable carrier.

483. A method of increasing the concentration of a target protein in a cell comprising delivery of a compound of any one of embodiments 1-481, or a pharmaceutically acceptable salt thereof.

484. The method of embodiment 483, wherein the target protein is the wild type protein.

485. The method of embodiment 483, wherein the target protein is a mutant protein.

486. A method of removing ubiquitin from a target protein comprising delivery of a compound of any one of embodiments 1-481, or a pharmaceutically acceptable salt thereof.

487. The method of embodiment 486, wherein the target protein is a natural target of USP28.

488. The method of embodiment 486, wherein the target protein is not a natural target of USP28.

489. A method of preventing or reducing the degradation of a target protein in a cell comprising delivering a compound of any one of embodiments 1-481, or a pharmaceutically acceptable salt thereof.

490. A method of treating or ameliorating a disease mediated by a target protein comprising delivery of a compound of any one of embodiments 1-481, or a pharmaceutically acceptable salt thereof.

491. The method of embodiment 490, wherein treatment or amelioration of the disease comprises removing ubiquitin from the target protein.

492. The method of embodiment 490, wherein treatment or amelioration of the disease comprises increasing the concentration of the target protein in a cell.

In certain embodiments the BAX stabilizing compound of the present invention is selected from:

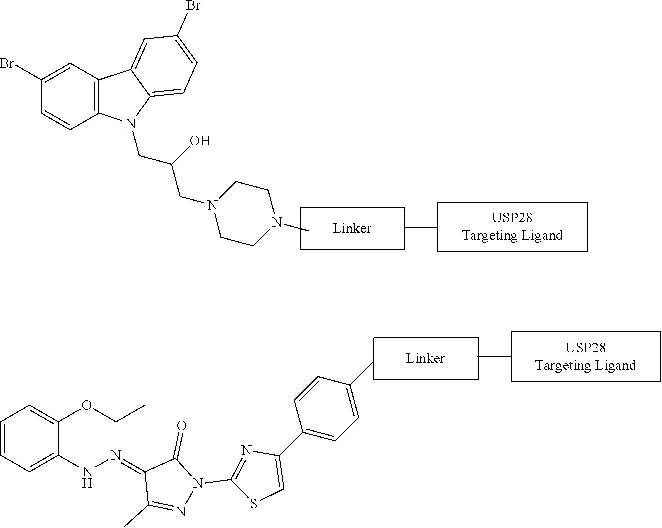

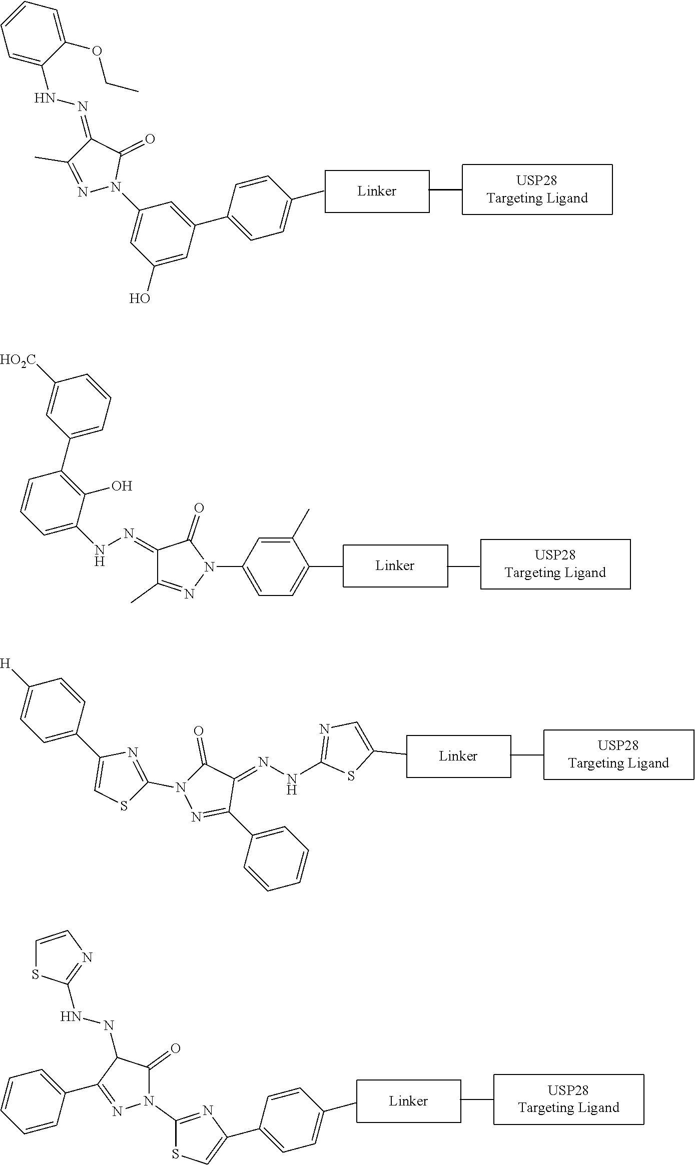

or a pharmaceutically acceptable salt thereof.

In certain embodiments the PKLR stabilizing compound of the present invention is selected from:

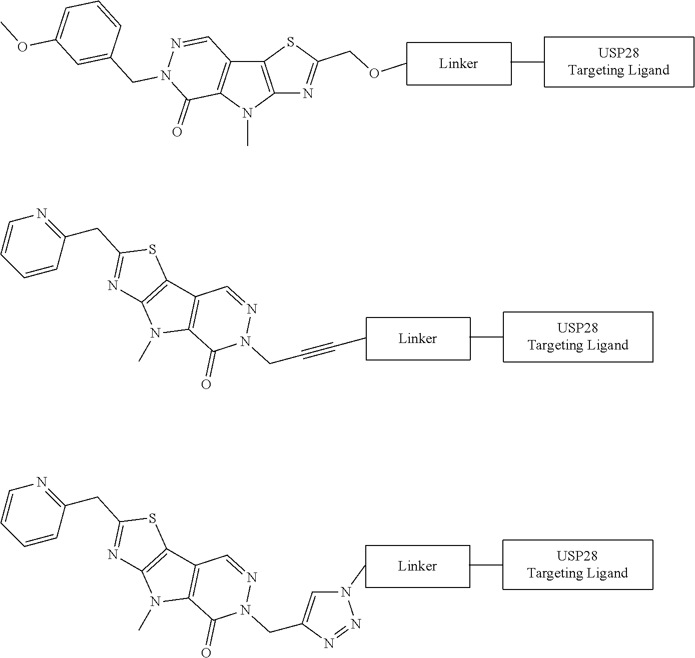

or a pharmaceutically acceptable salt thereof.

In certain embodiments the KEAP1 stabilizing compound of the present invention is selected from:

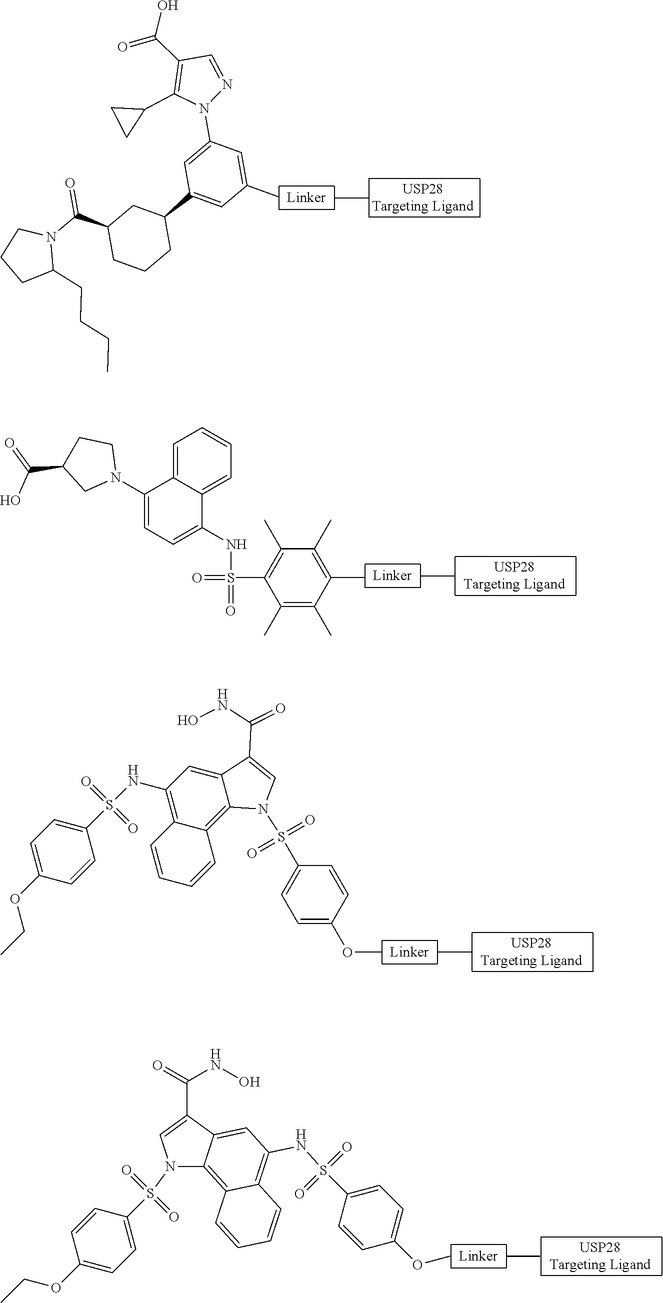

or a pharmaceutically acceptable salt thereof.

In certain embodiments the IRAK4 stabilizing compound of the present invention is selected from:

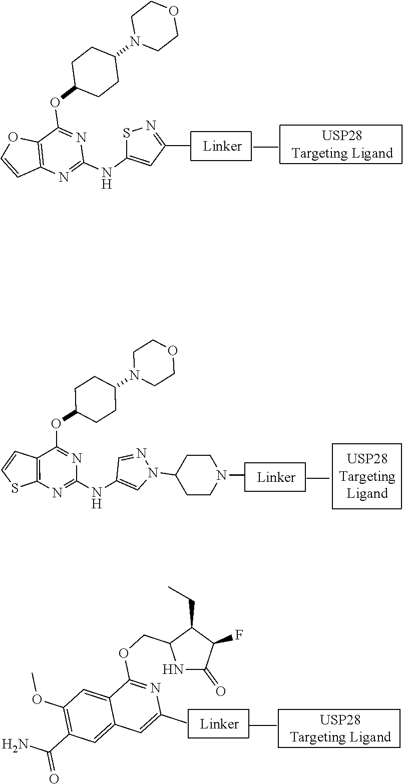

or a pharmaceutically acceptable salt thereof.

In certain embodiments the PTEN stabilizing compound of the present invention is selected from:

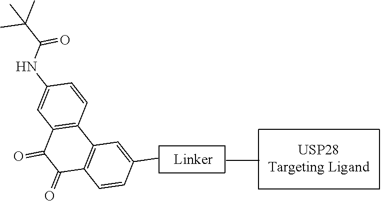

or a pharmaceutically acceptable salt thereof.

In certain embodiments the TK2 stabilizing compound of the present invention is selected from:

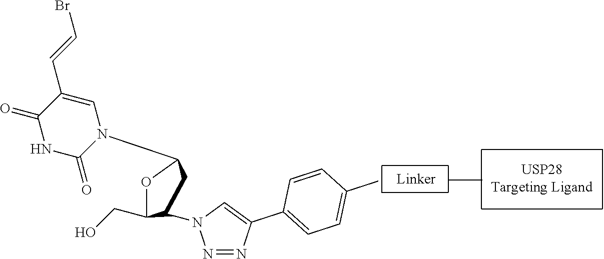

or a pharmaceutically acceptable salt thereof.

In certain embodiments the KCNQ1 stabilizing compound of the present invention is selected from:

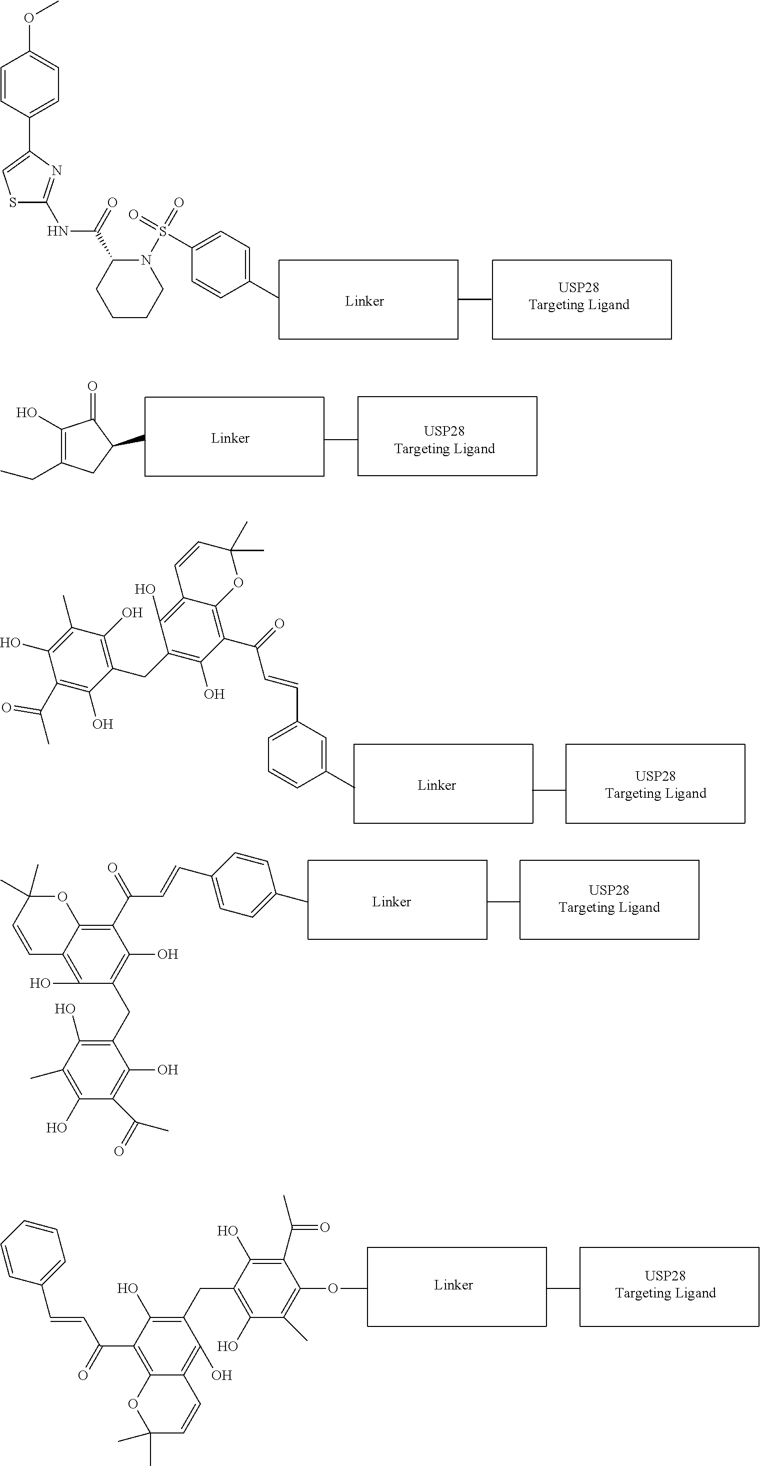

or a pharmaceutically acceptable salt thereof.

In certain embodiments the compound of the present invention is selected from:

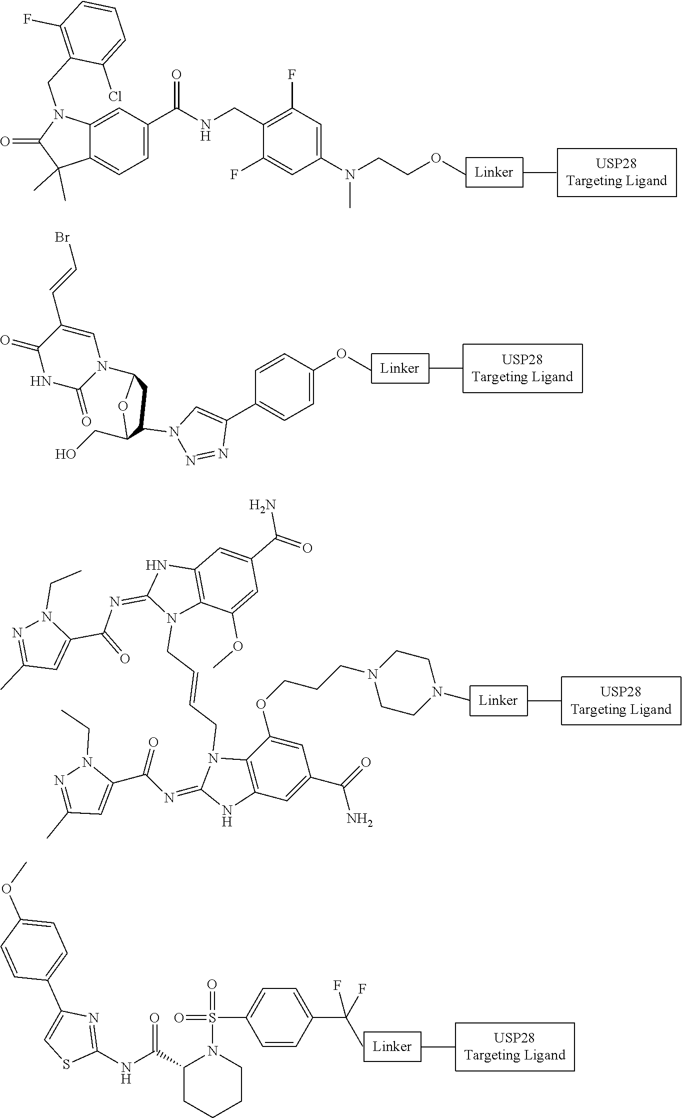

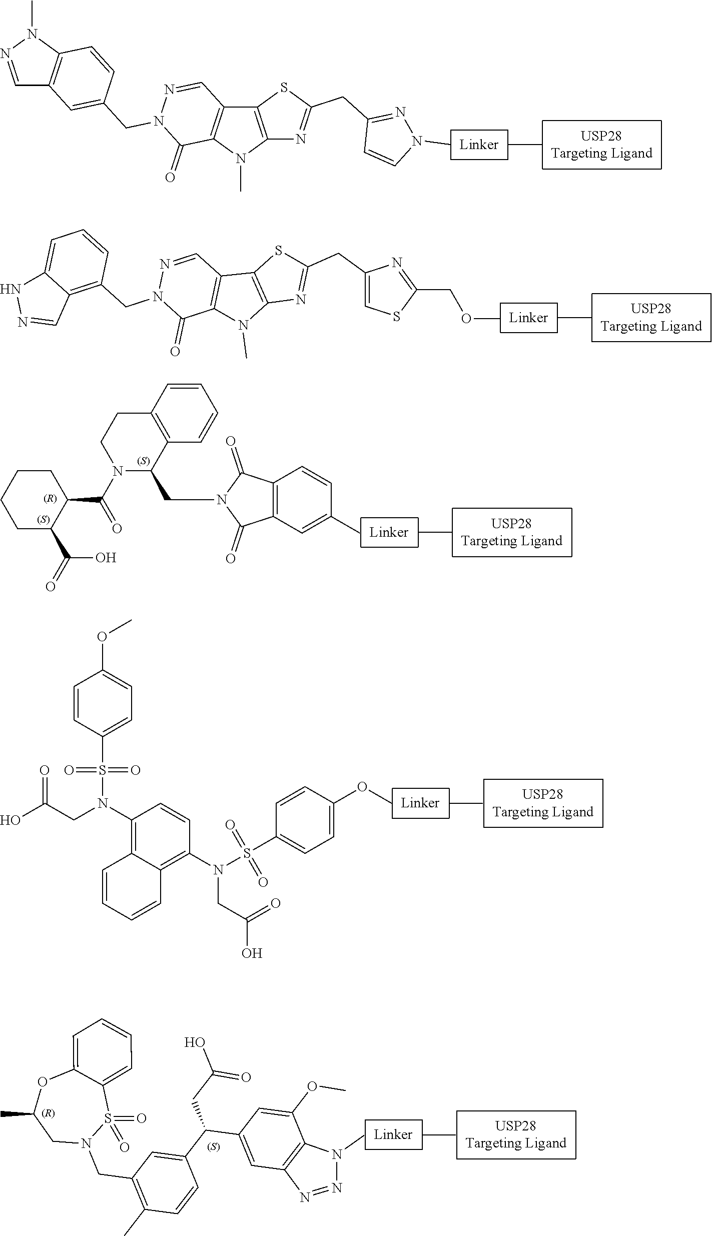

or a pharmaceutically acceptable salt thereof.

In certain embodiments the compound of the present invention is selected from:

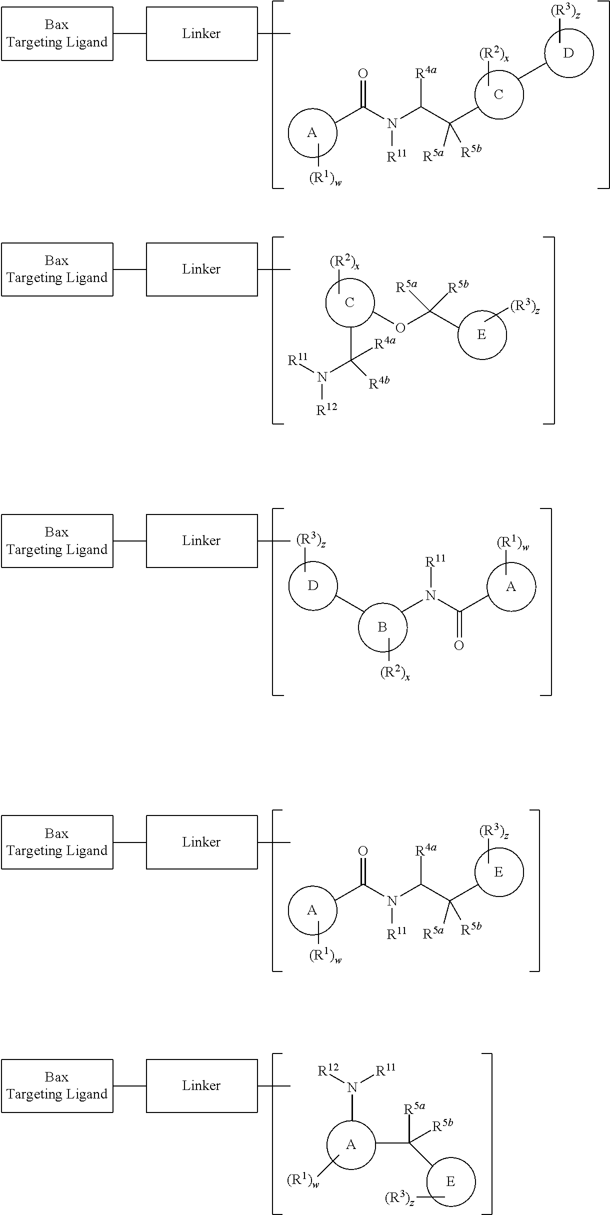

or a pharmaceutically acceptable salt thereof.

In certain embodiments the compound of the present invention is selected from:

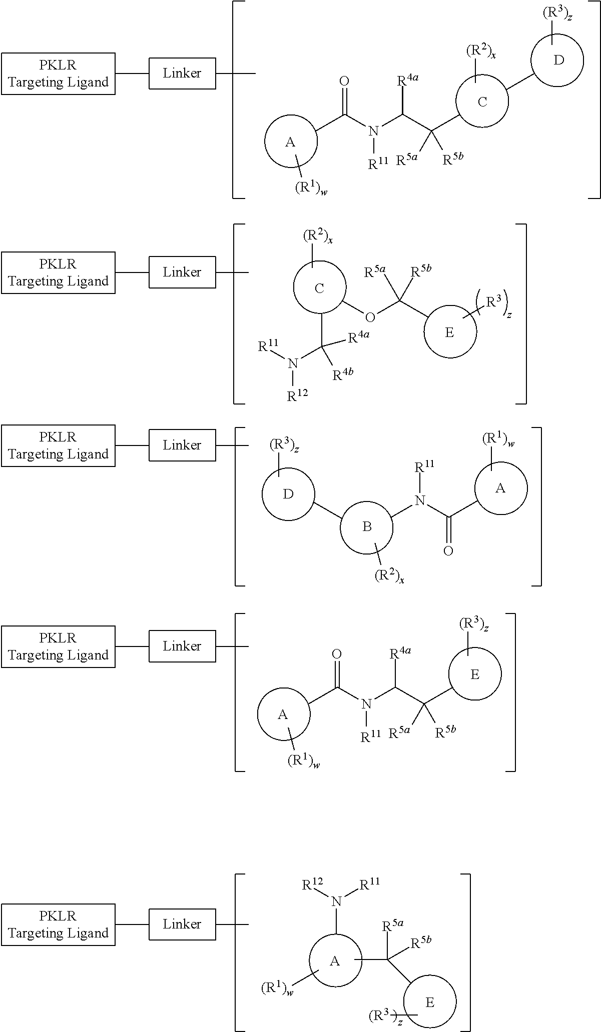

or a pharmaceutically acceptable salt thereof.

In certain embodiments the compound of the present invention is selected from:

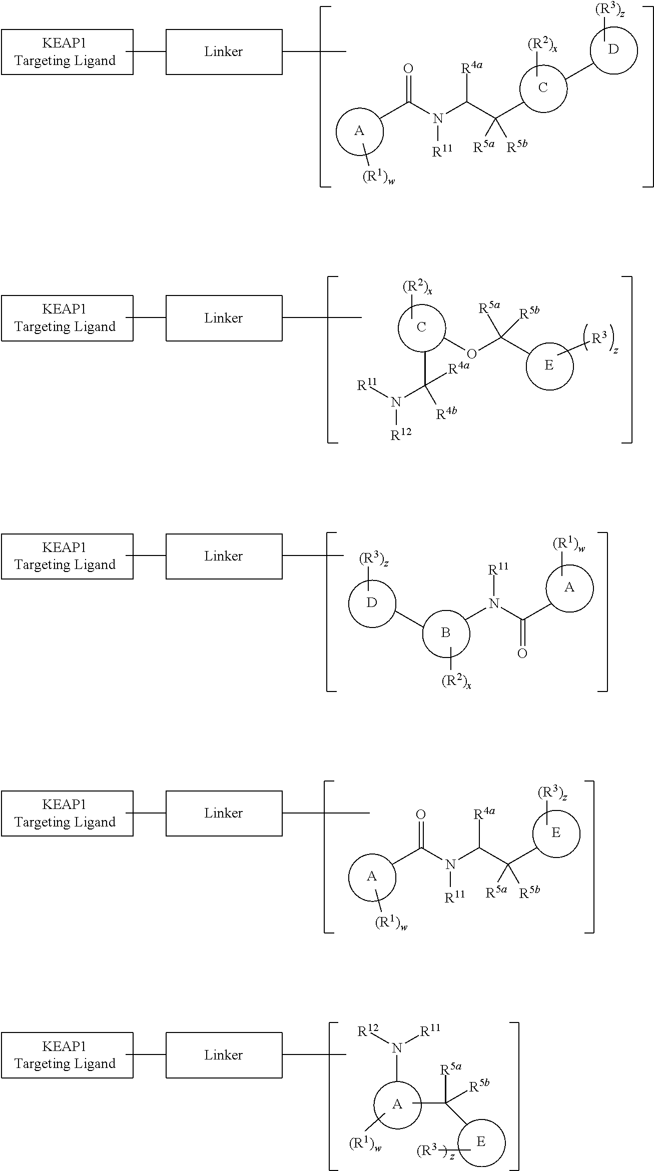

or a pharmaceutically acceptable salt thereof.

In certain embodiments the compound of the present invention is selected from:

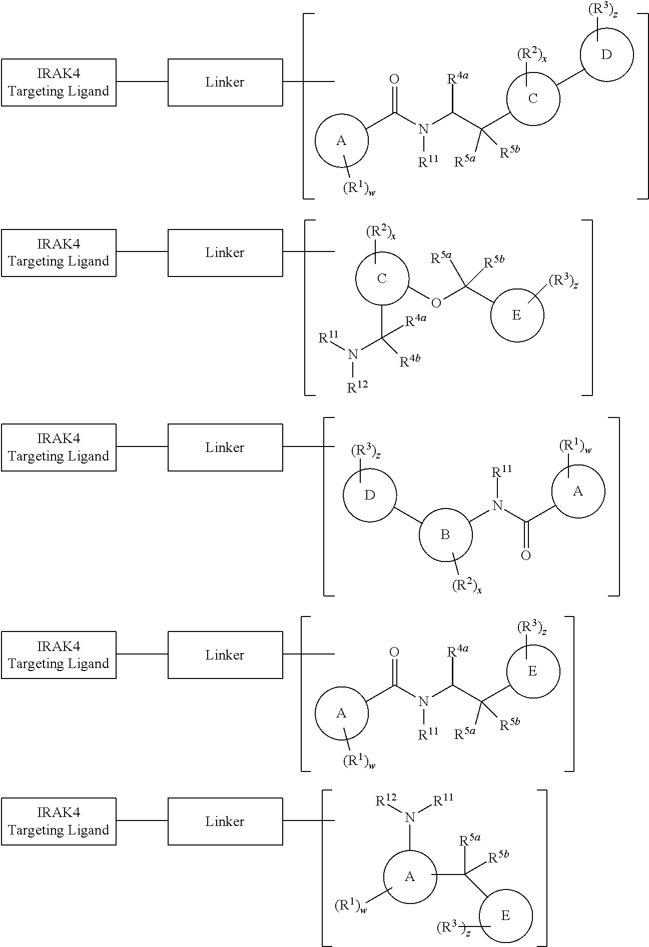

or a pharmaceutically acceptable salt thereof.

In certain embodiments the compound of the present invention is selected from:

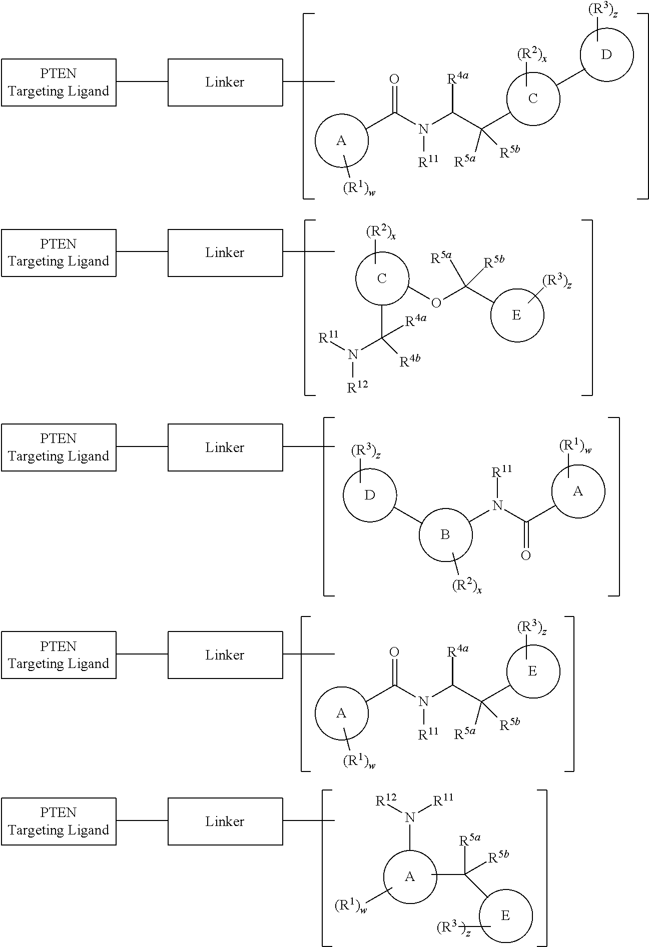

or a pharmaceutically acceptable salt thereof.

In certain embodiments the compound of the present invention is selected from:

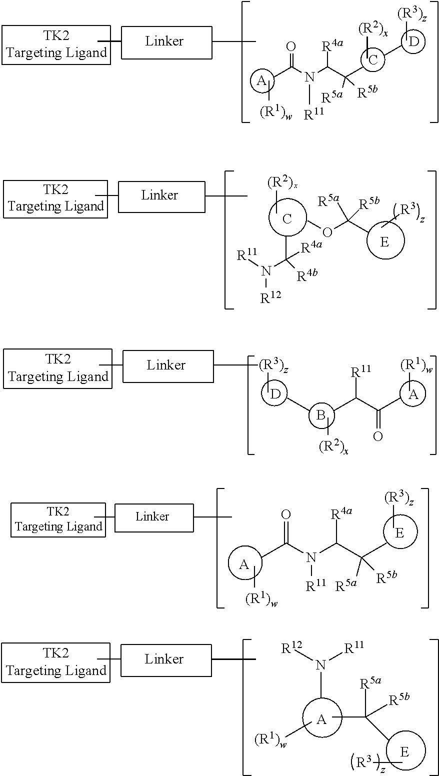

or a pharmaceutically acceptable salt thereof.

In certain embodiments the compound of the present invention is selected from:

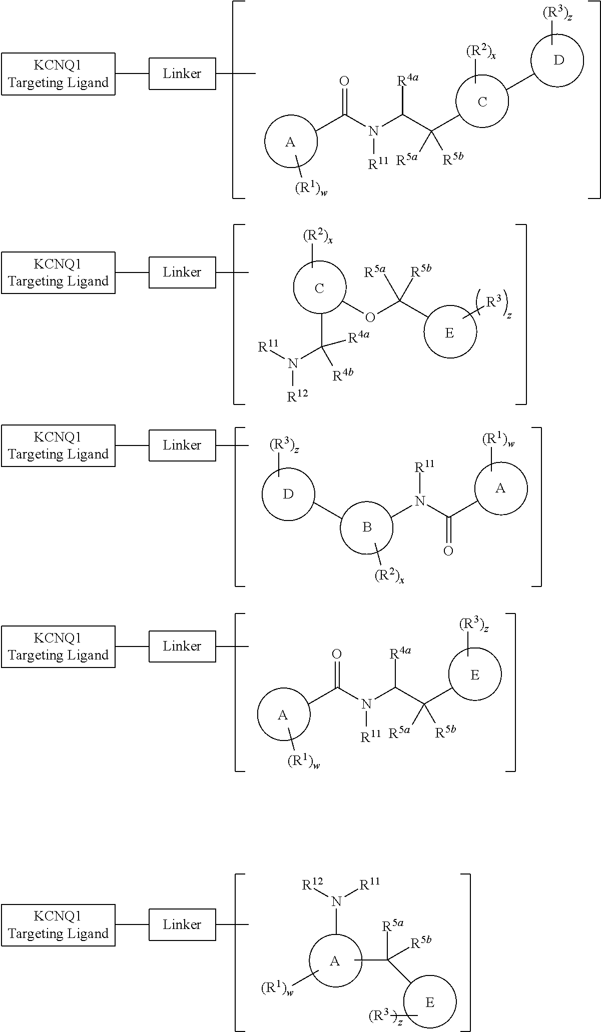

or a pharmaceutically acceptable salt thereof.

In certain embodiments the compound of the present invention is selected from:

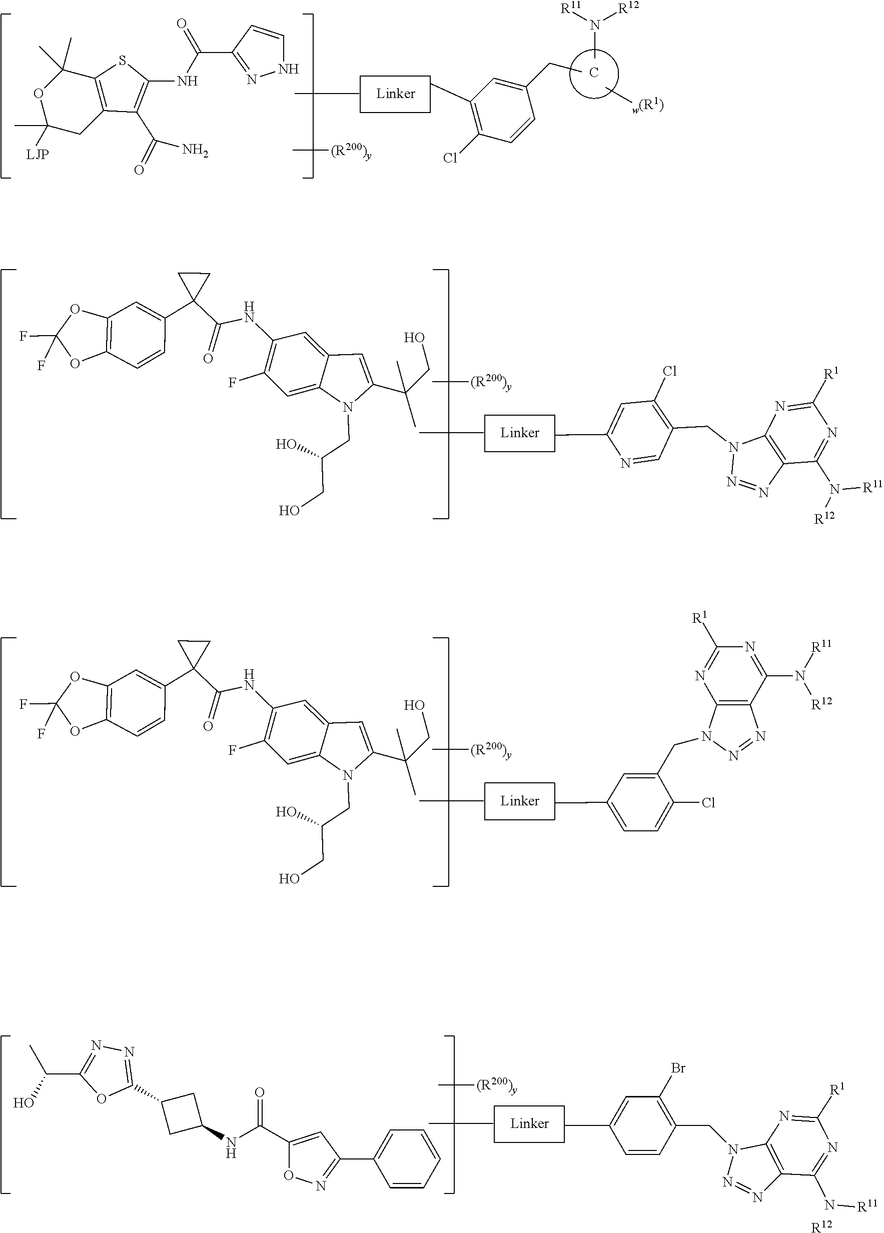

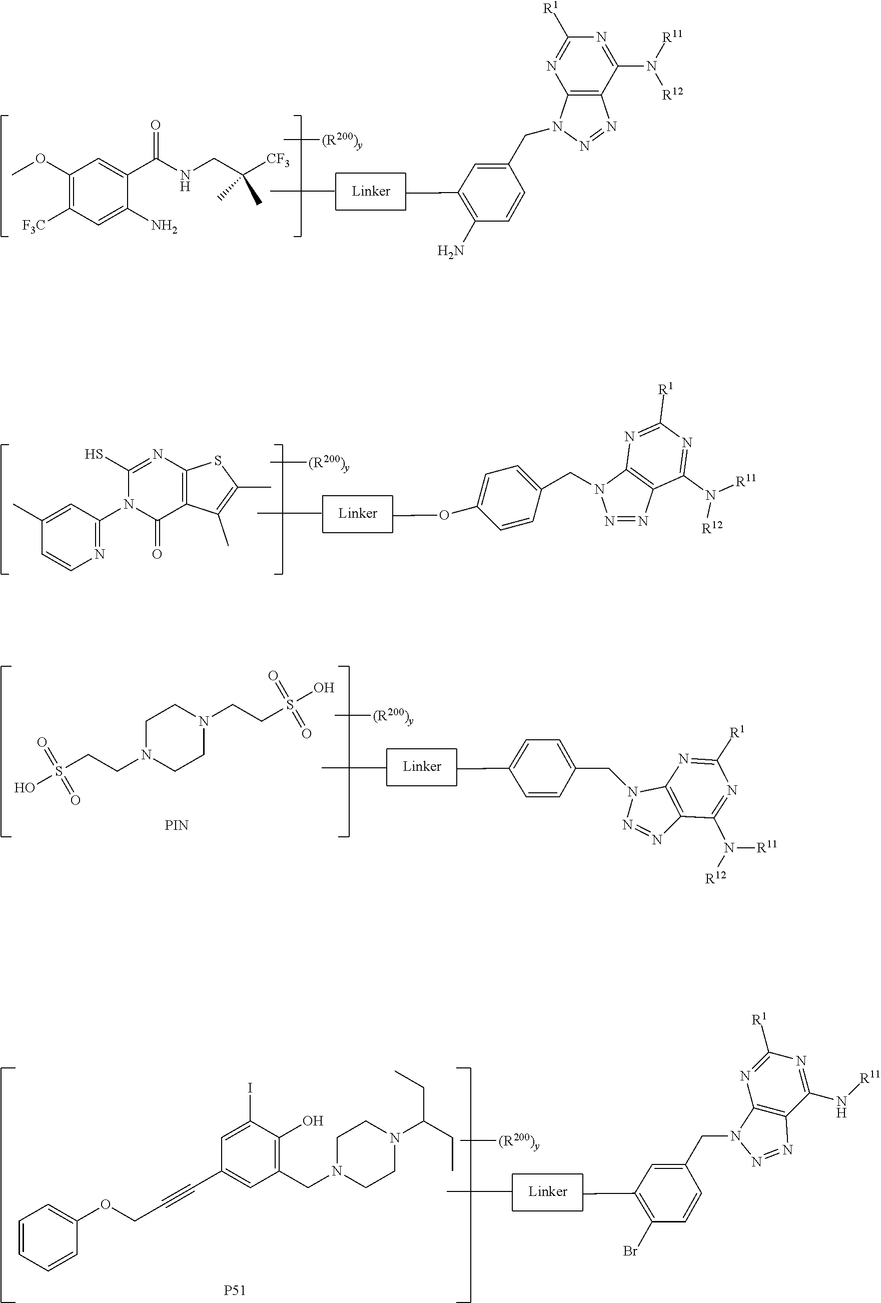

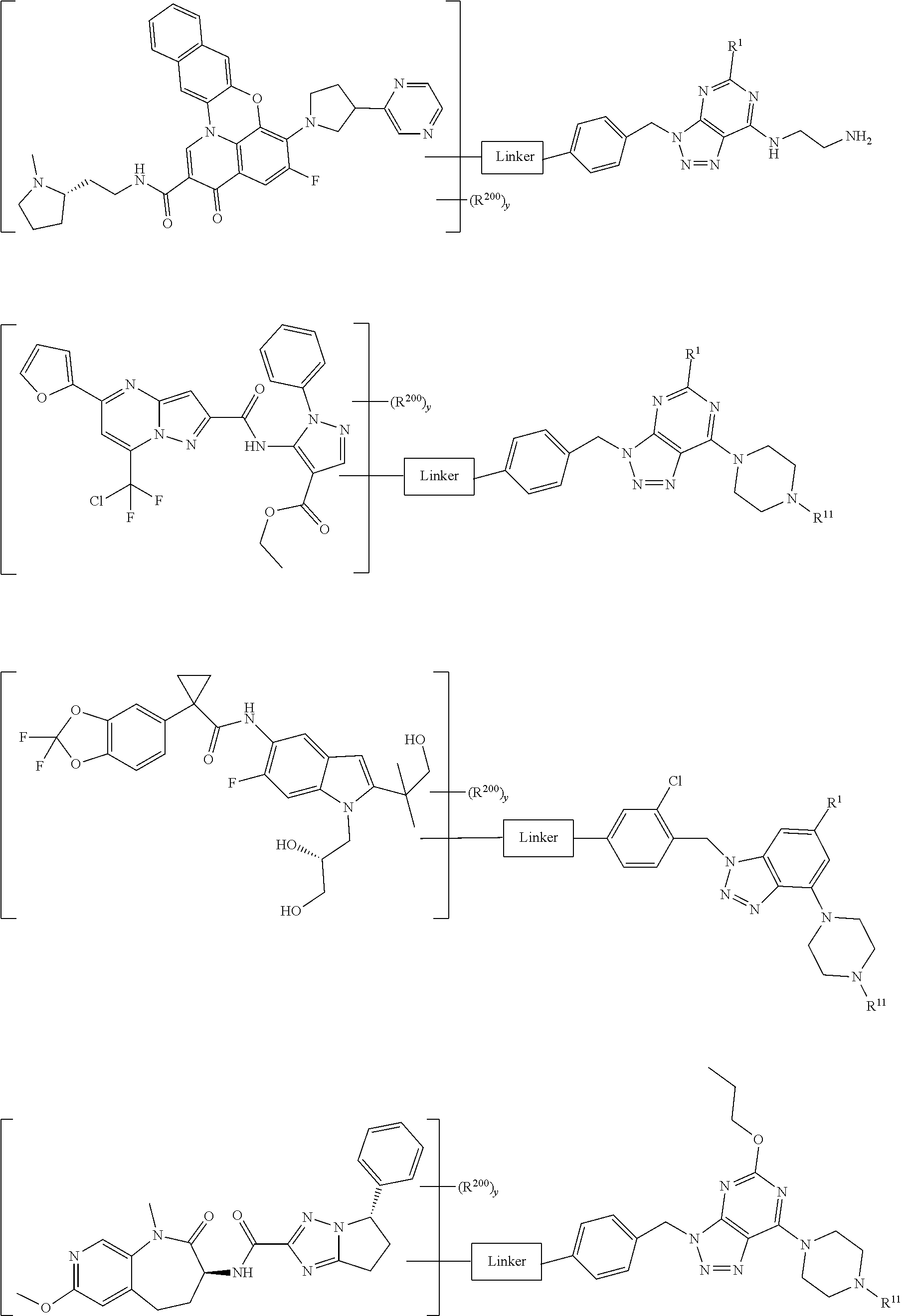

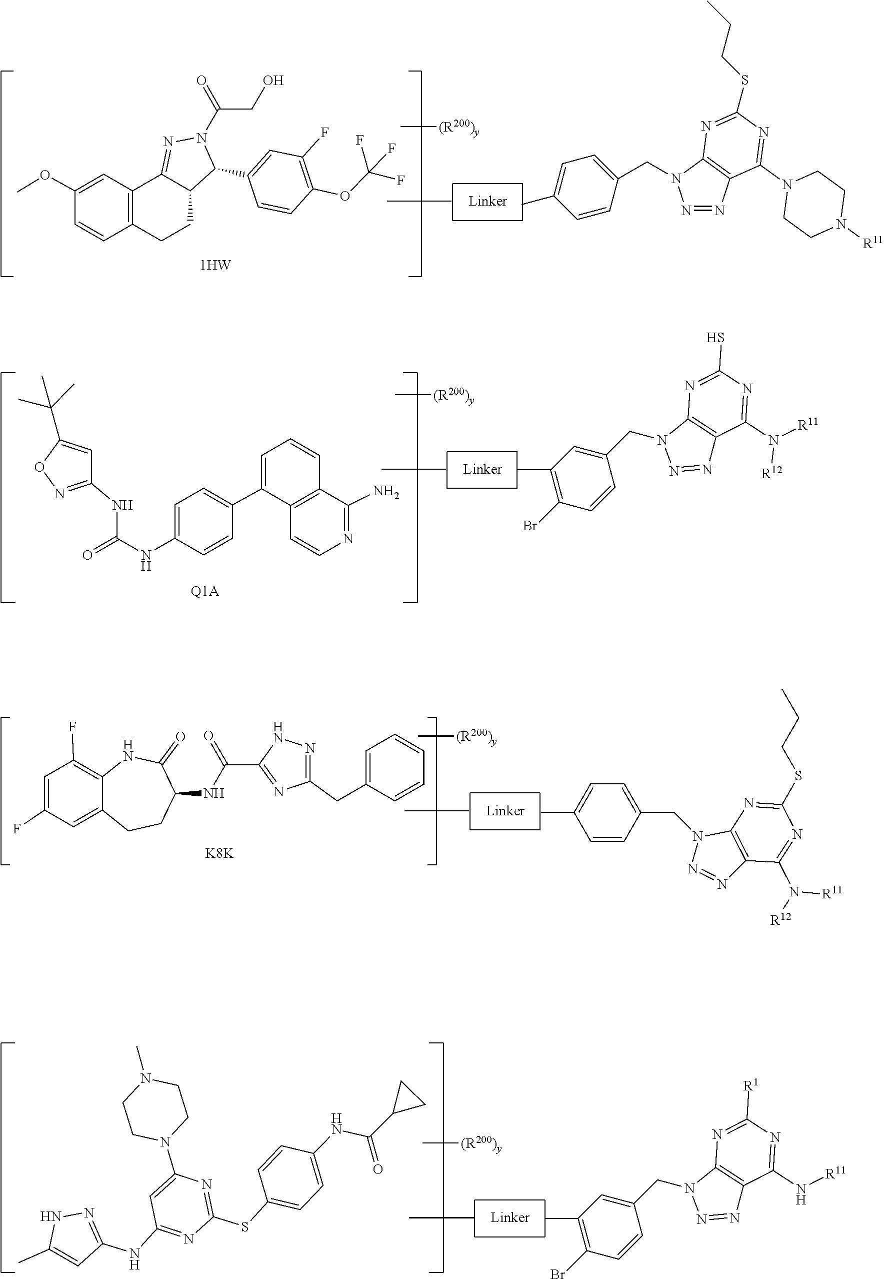

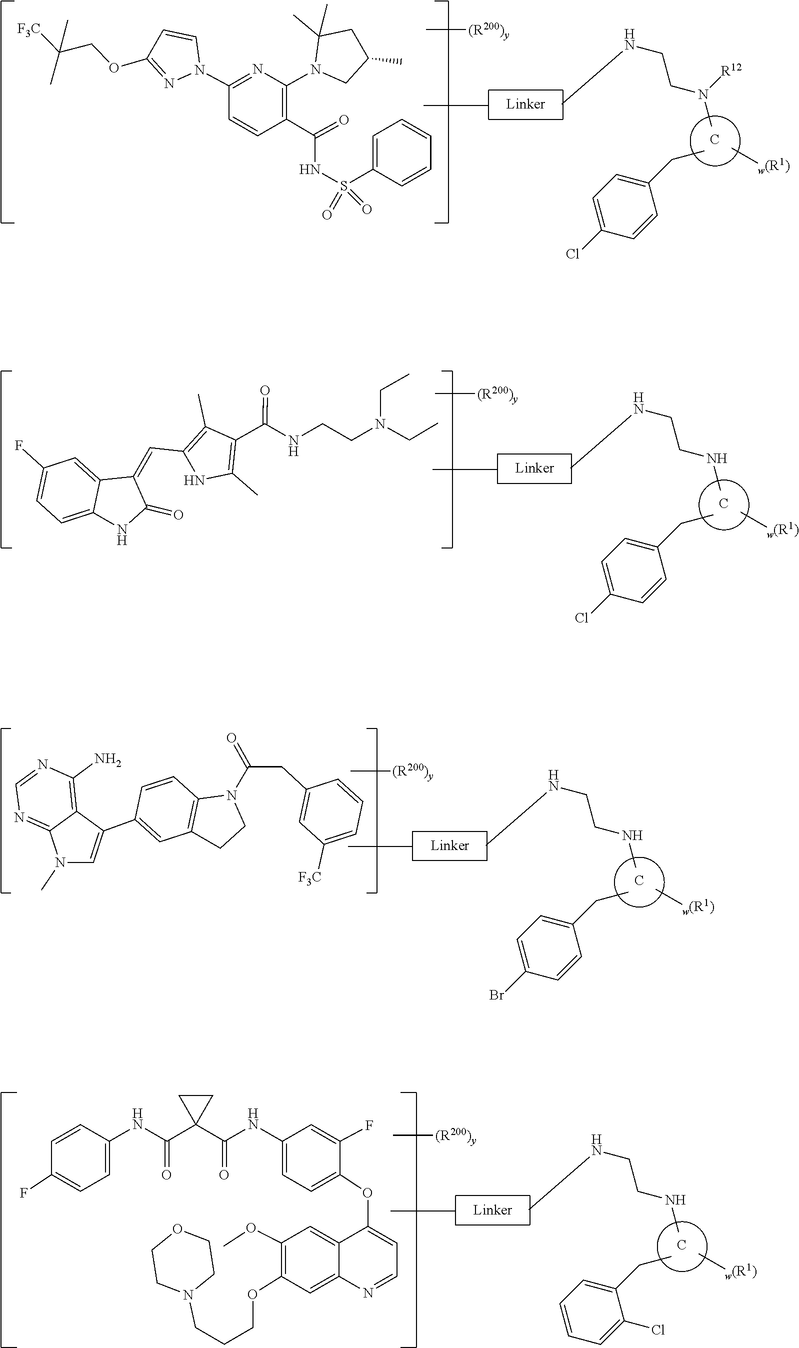

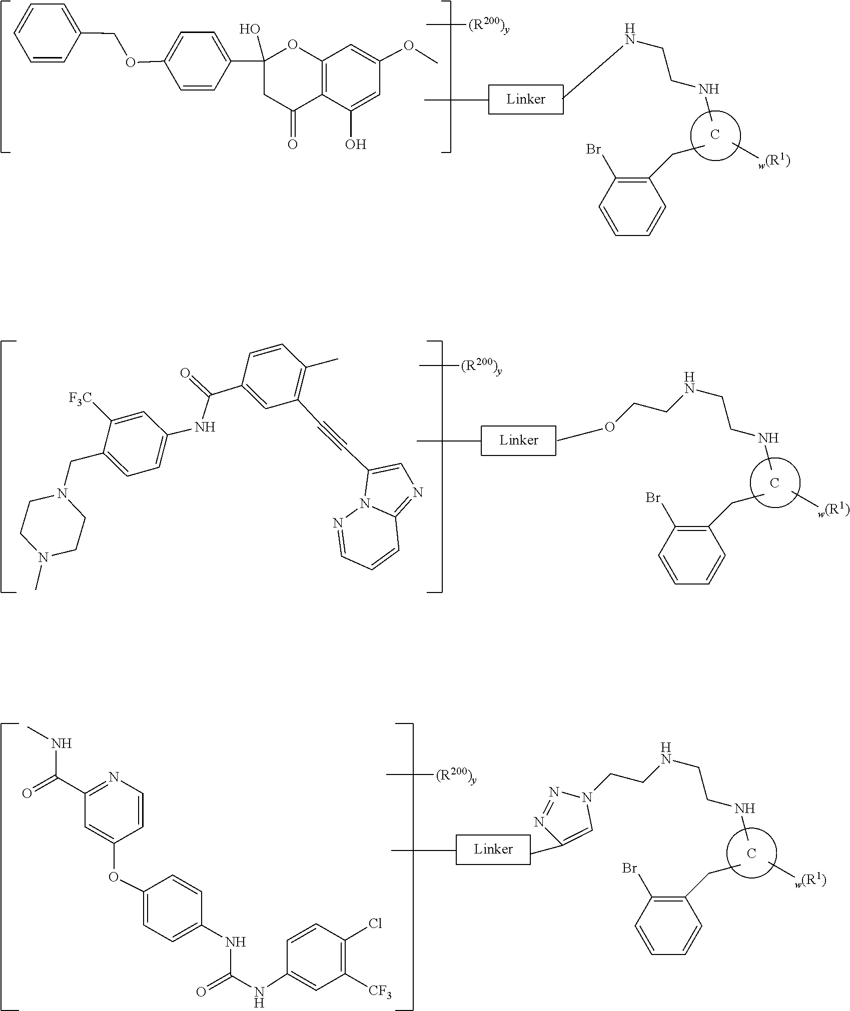

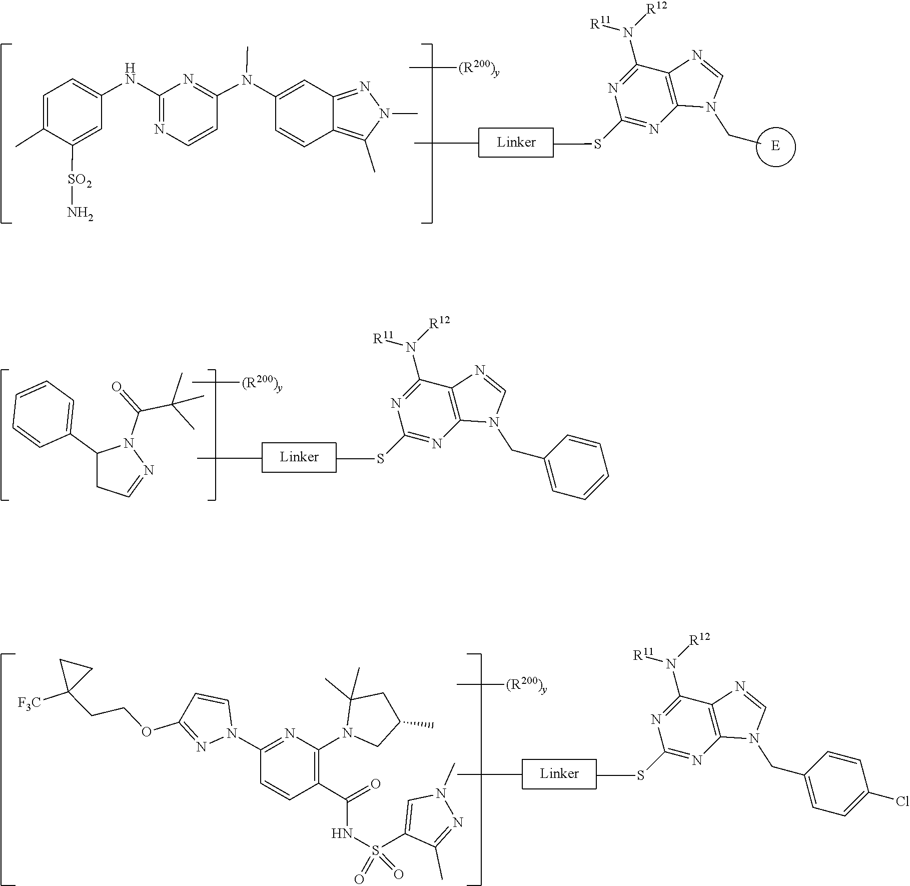

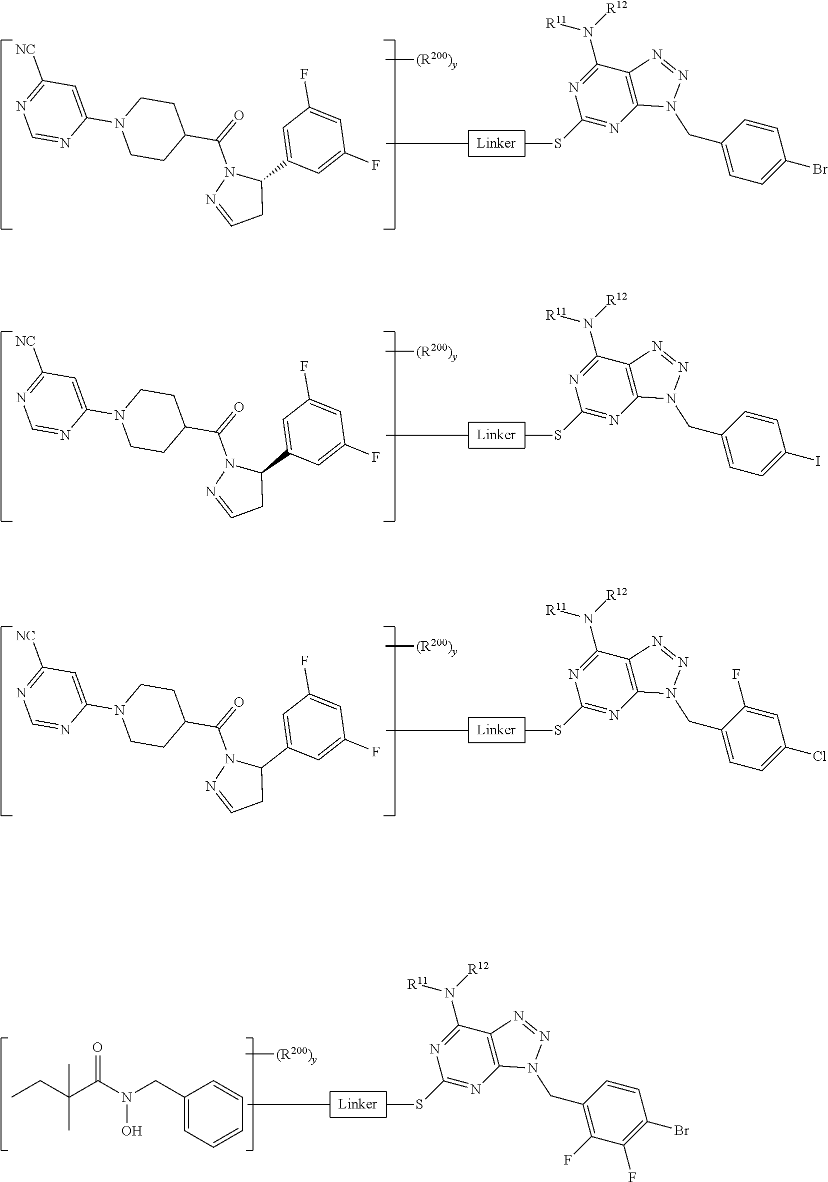

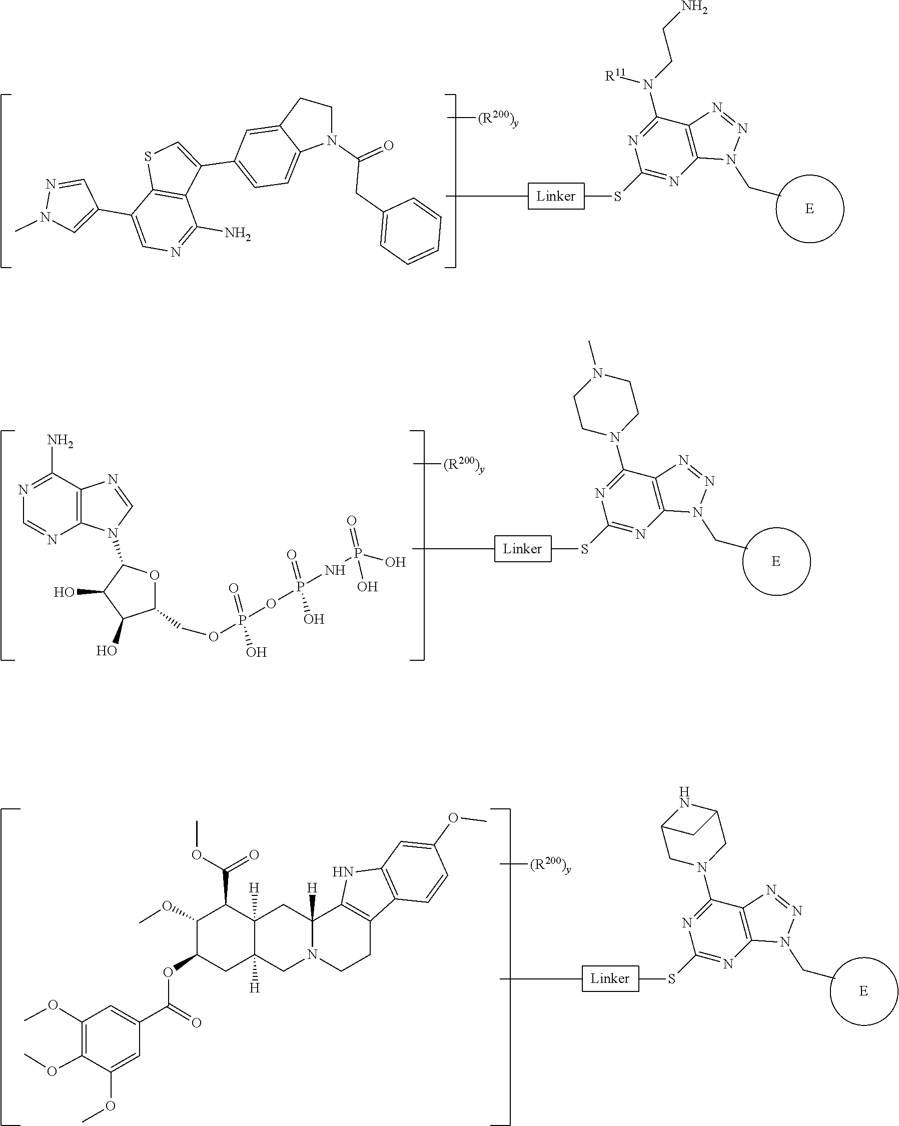

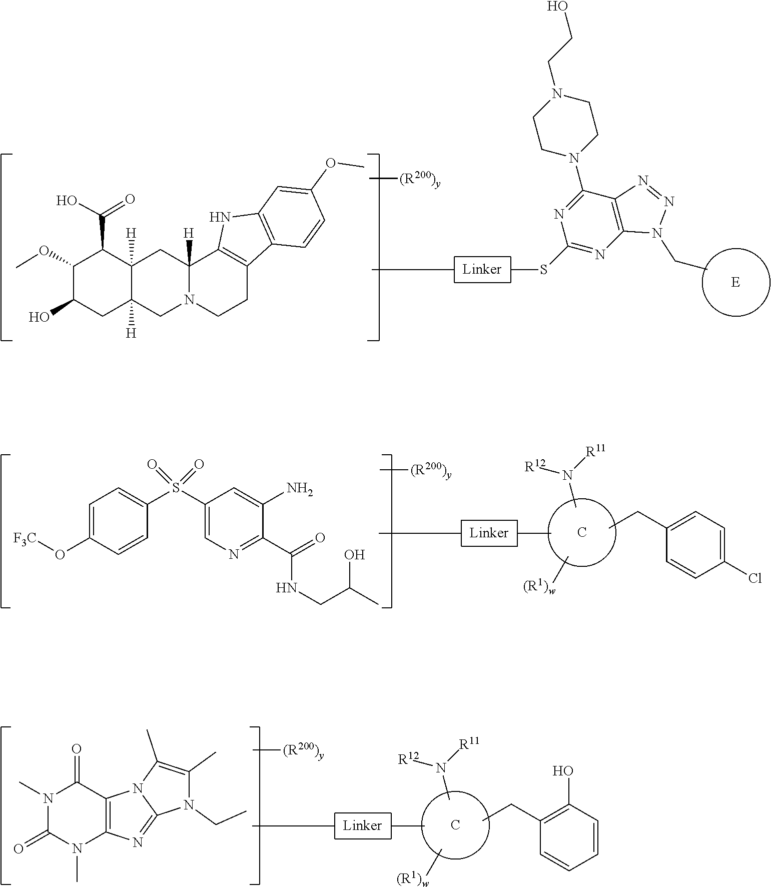

or a pharmaceutically acceptable salt thereof.

In certain embodiments the protein stabilizing compound of the present invention is selected from:

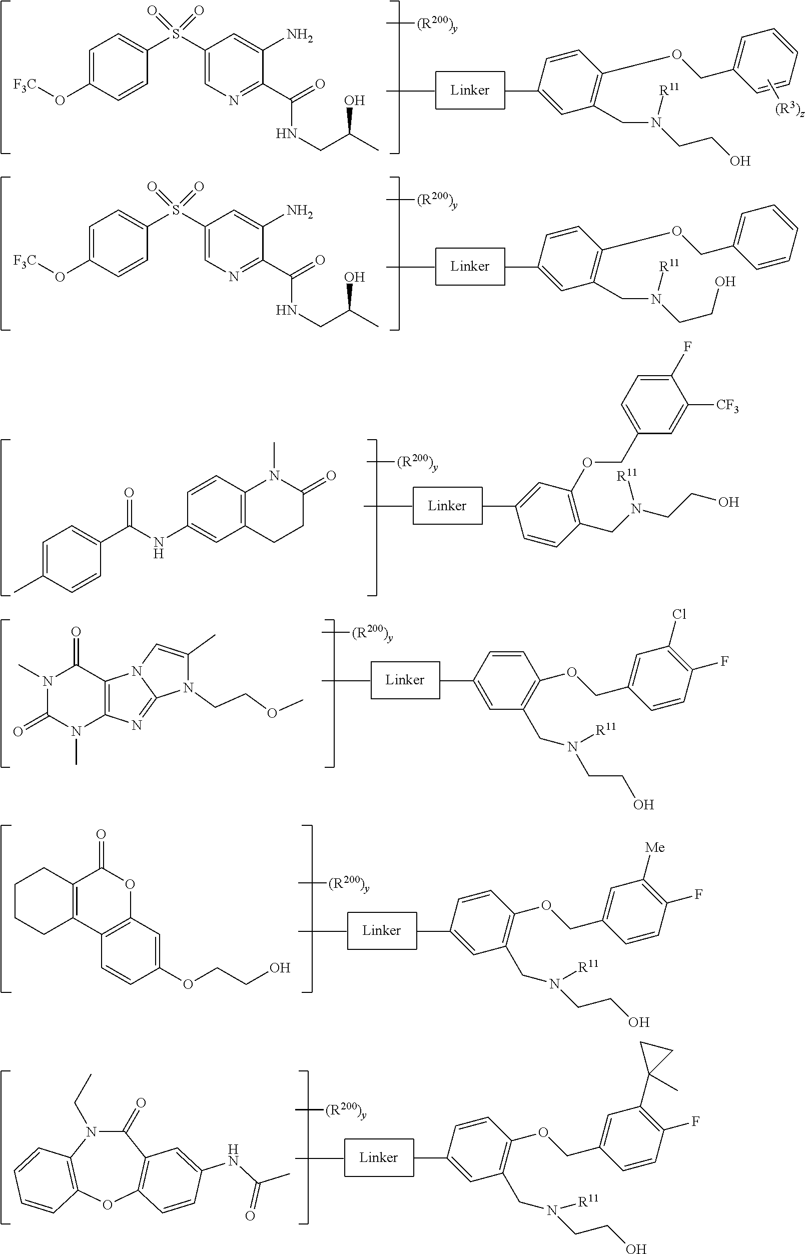

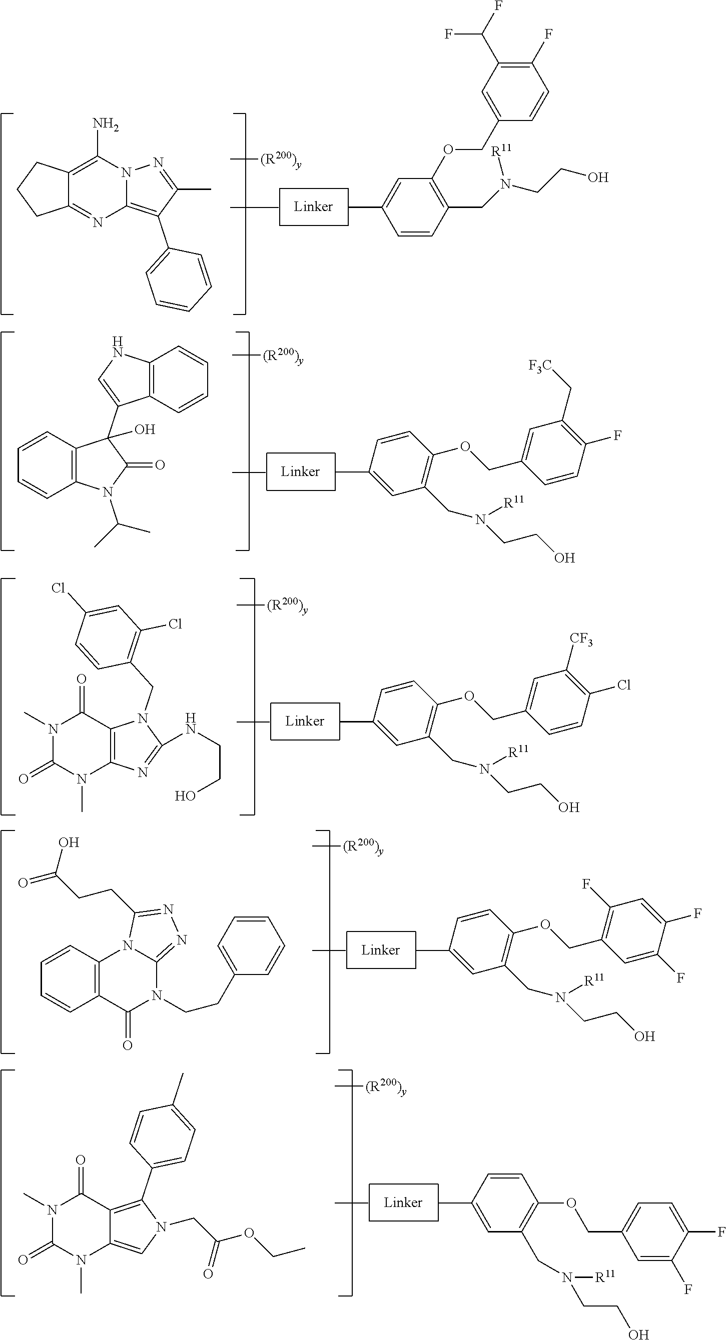

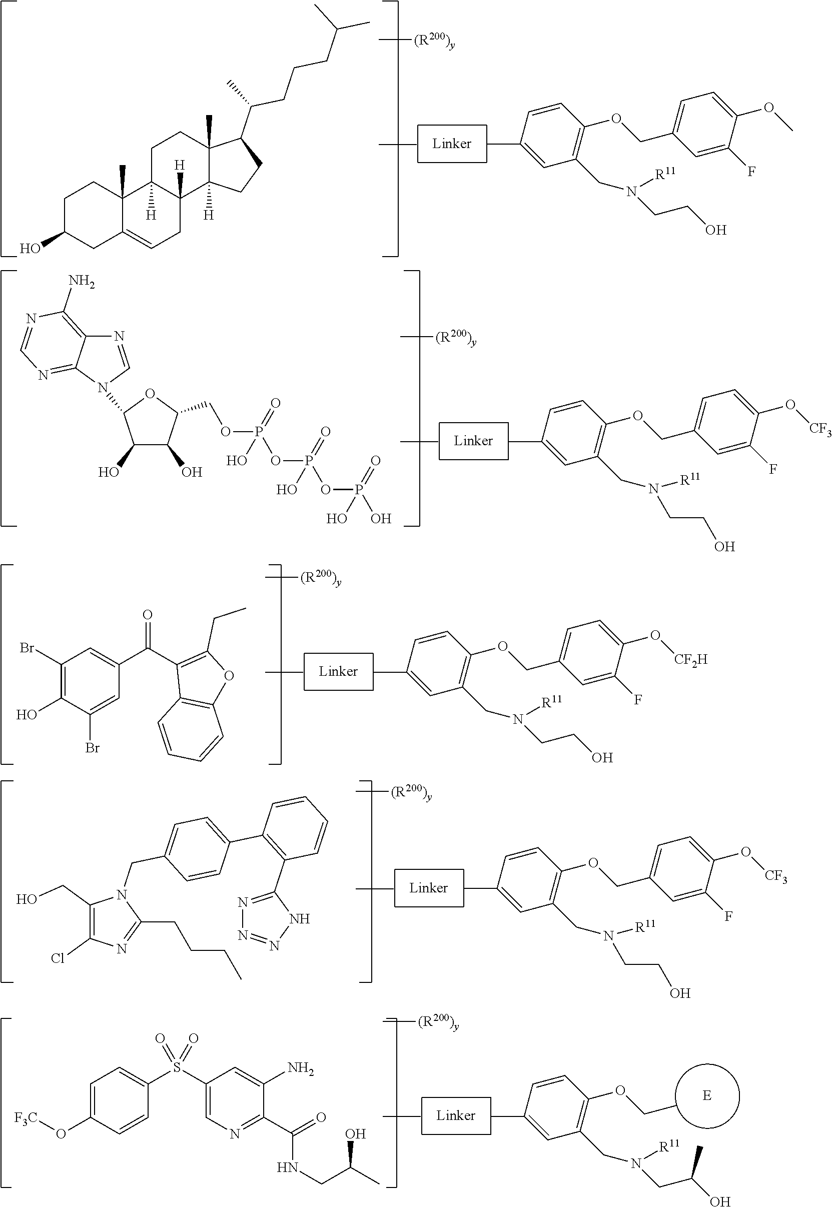

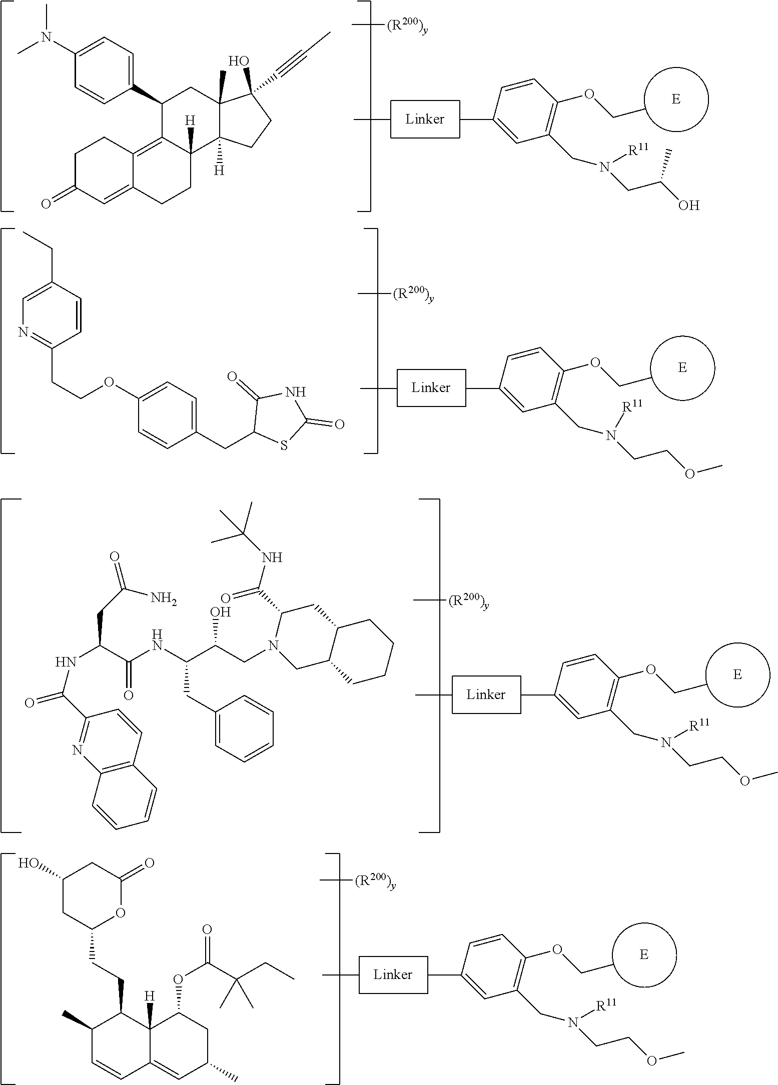

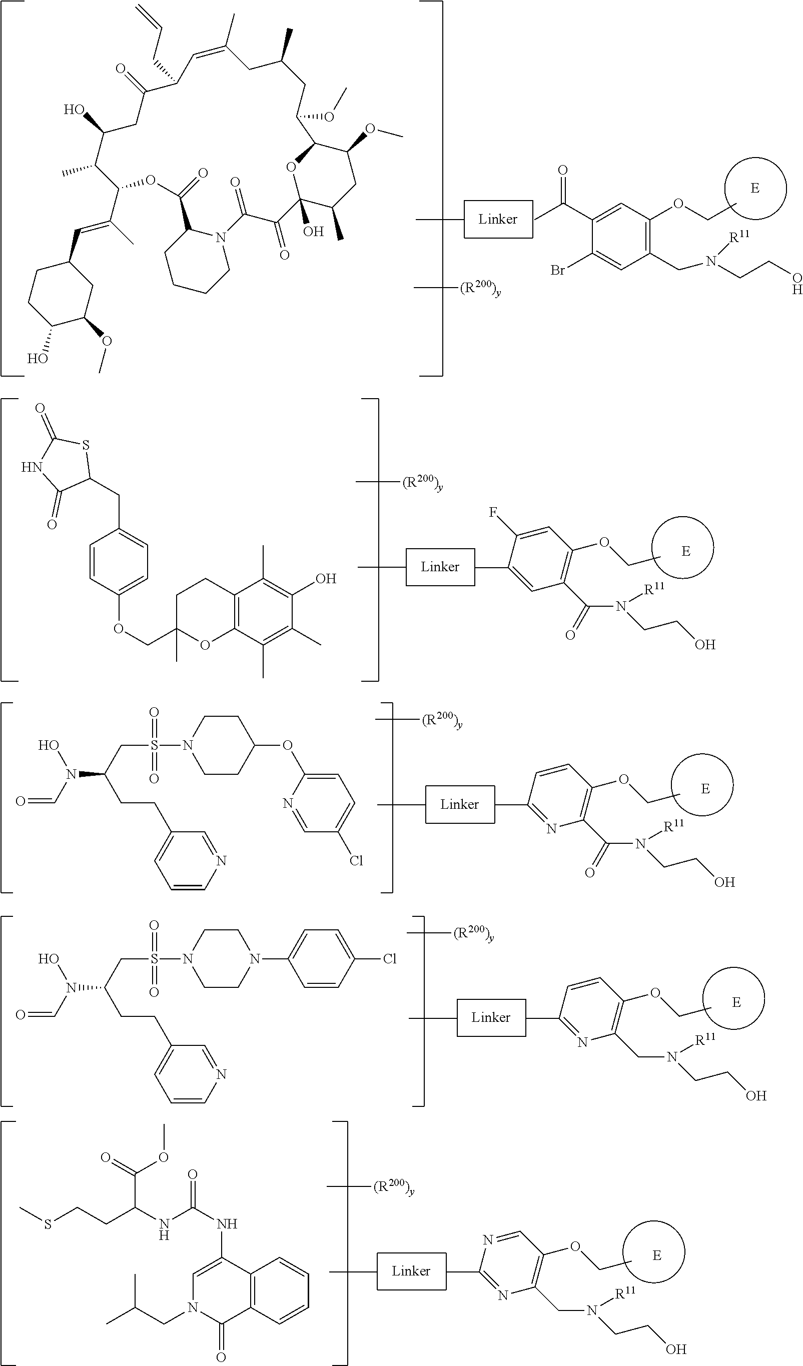

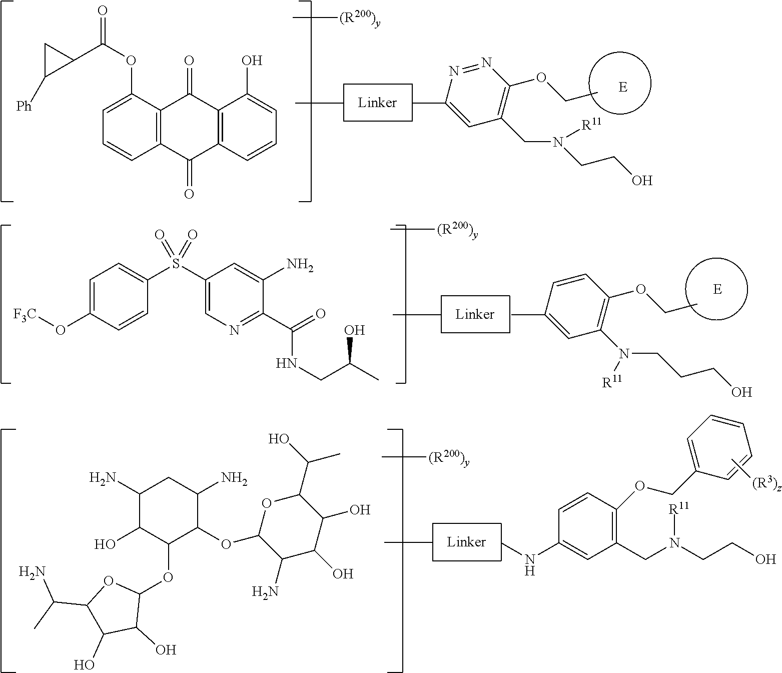

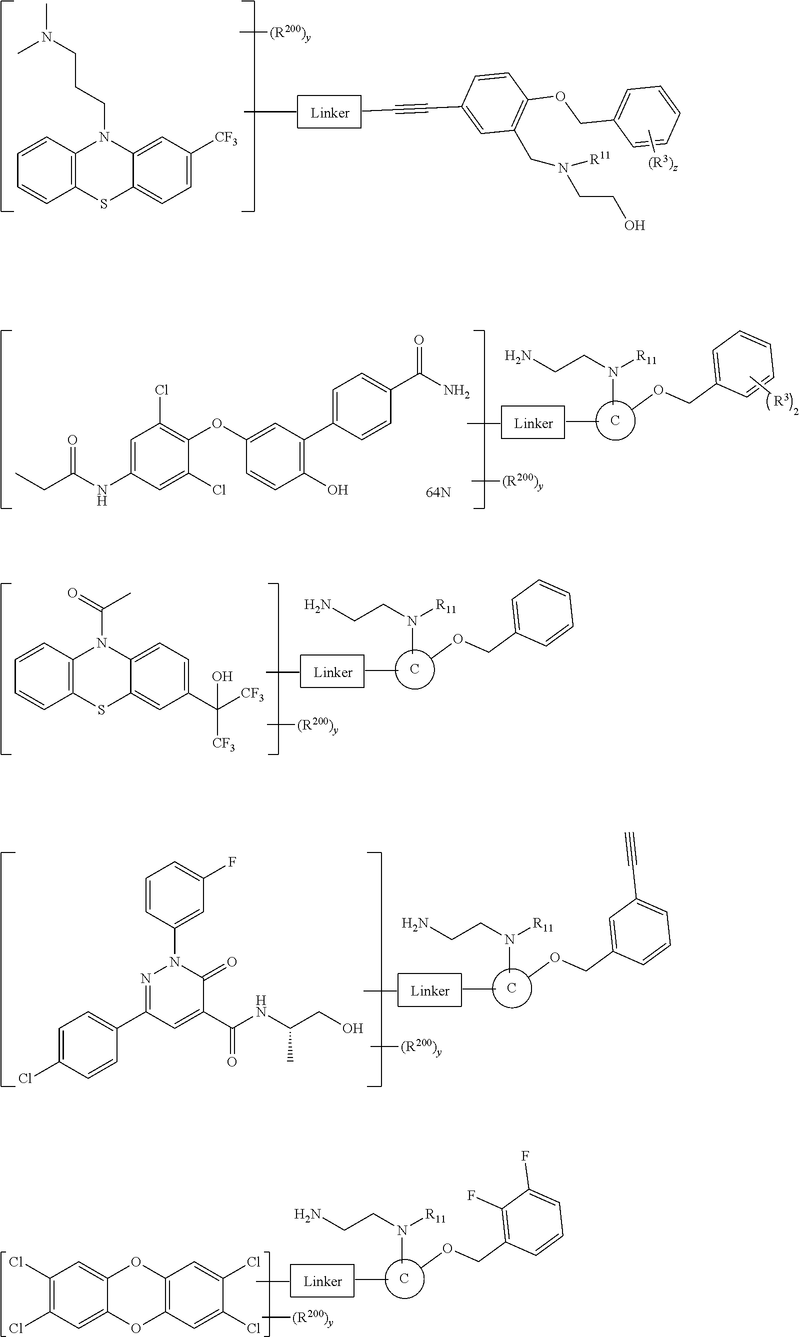

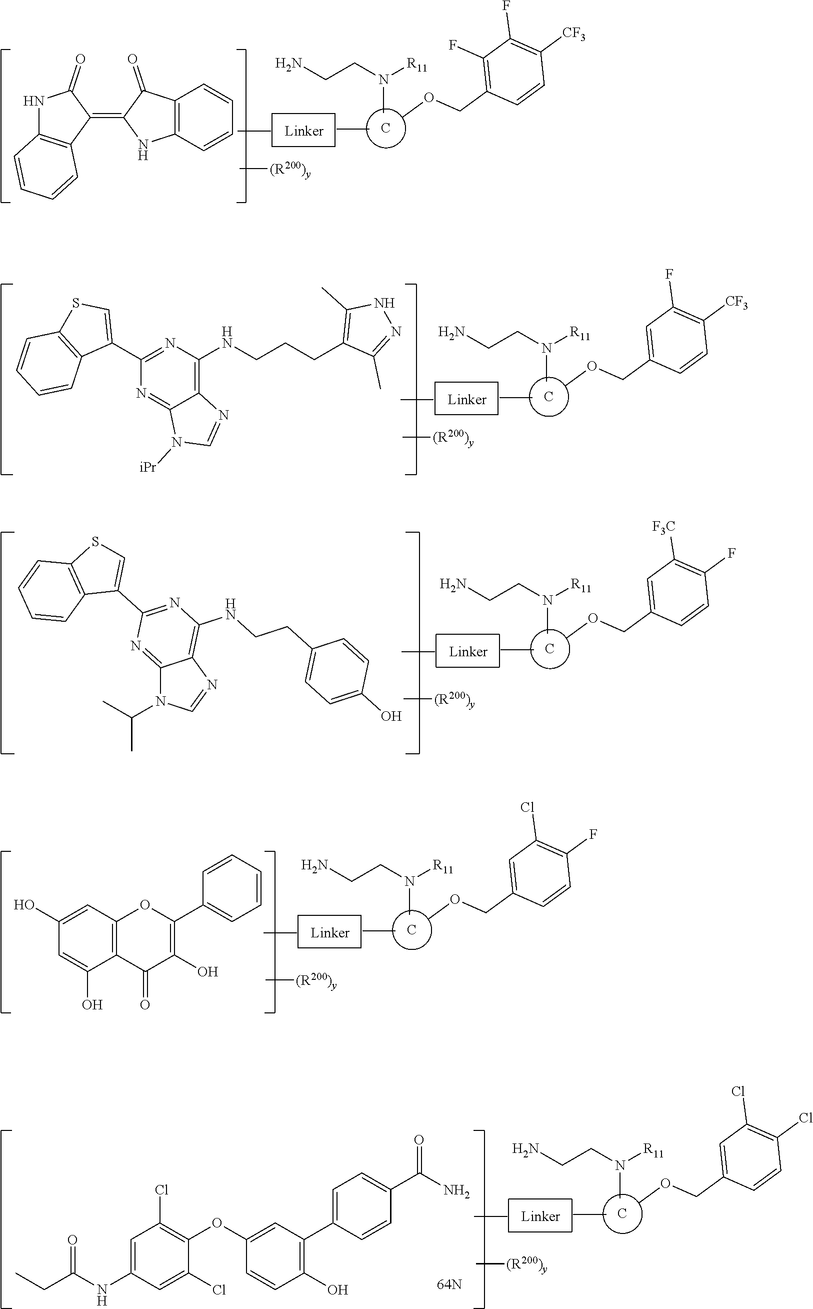

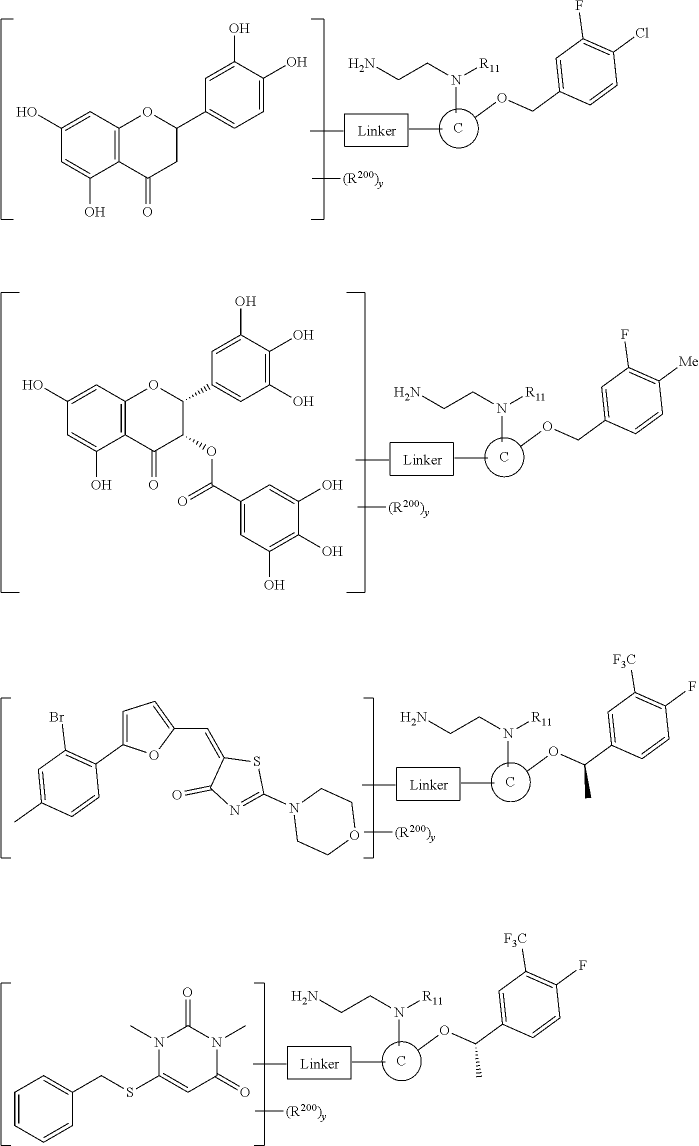

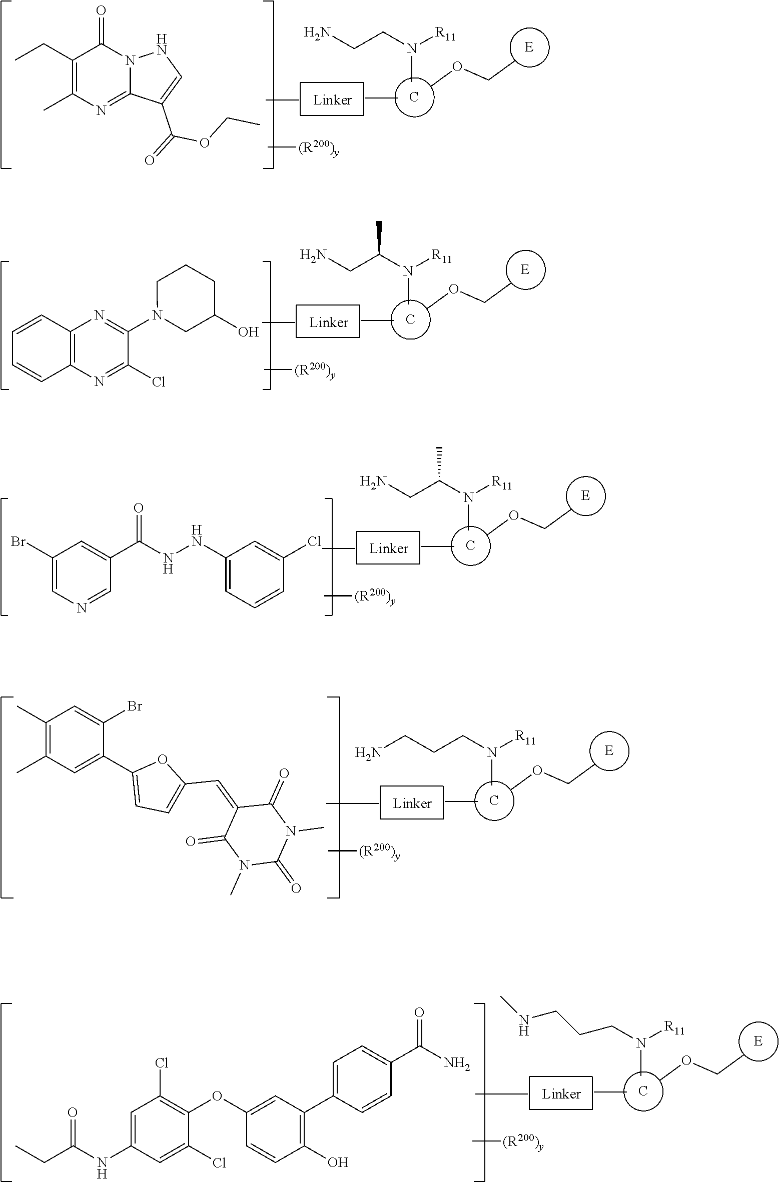

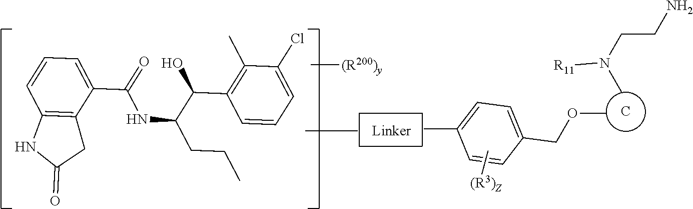

or a pharmaceutically acceptable salt thereof.

In certain embodiments the protein stabilizing compound of the present invention is selected from:

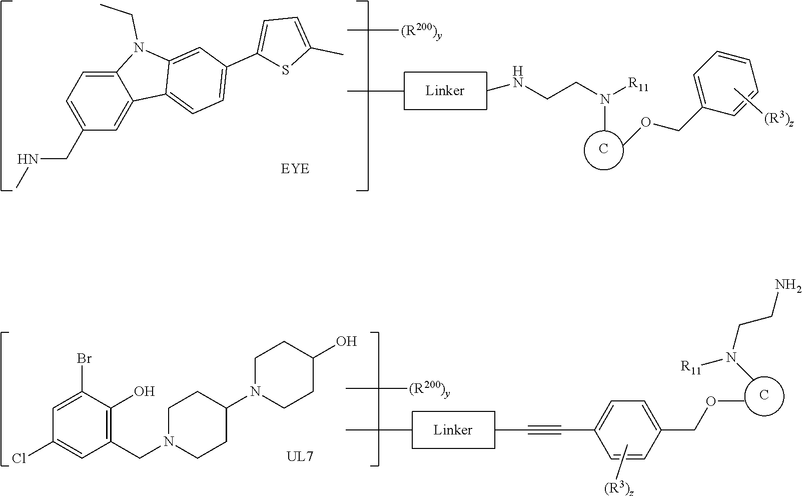

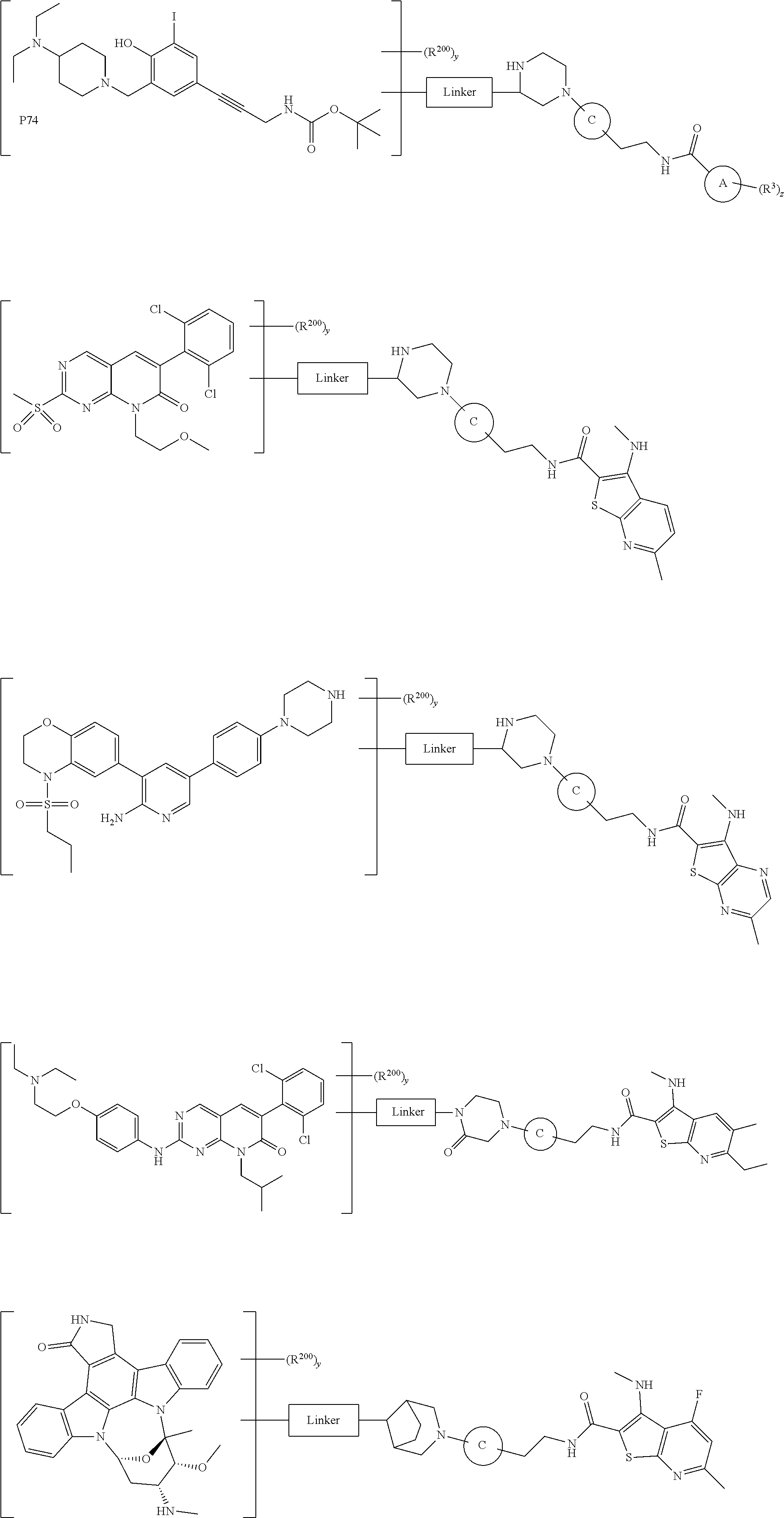

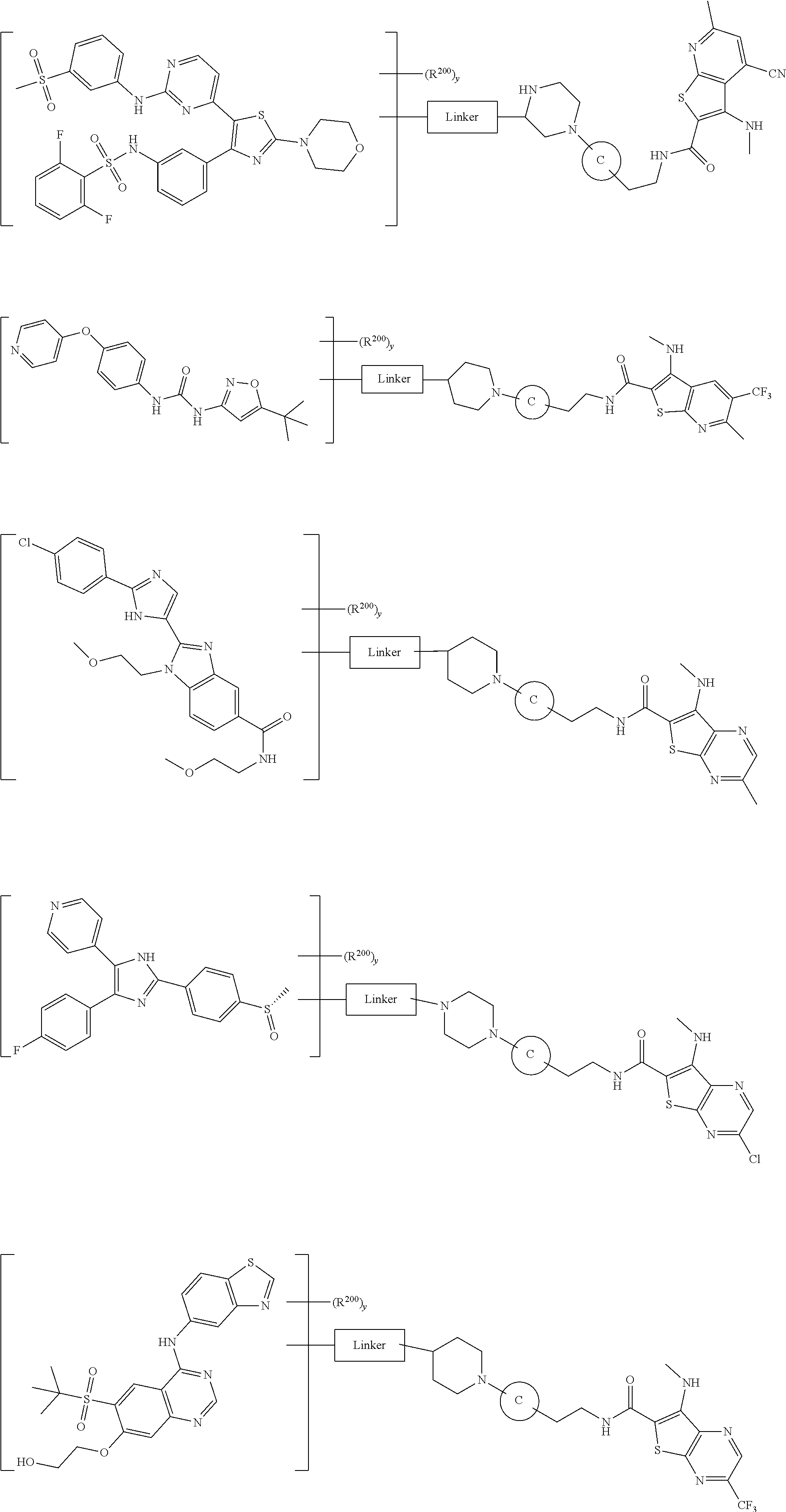

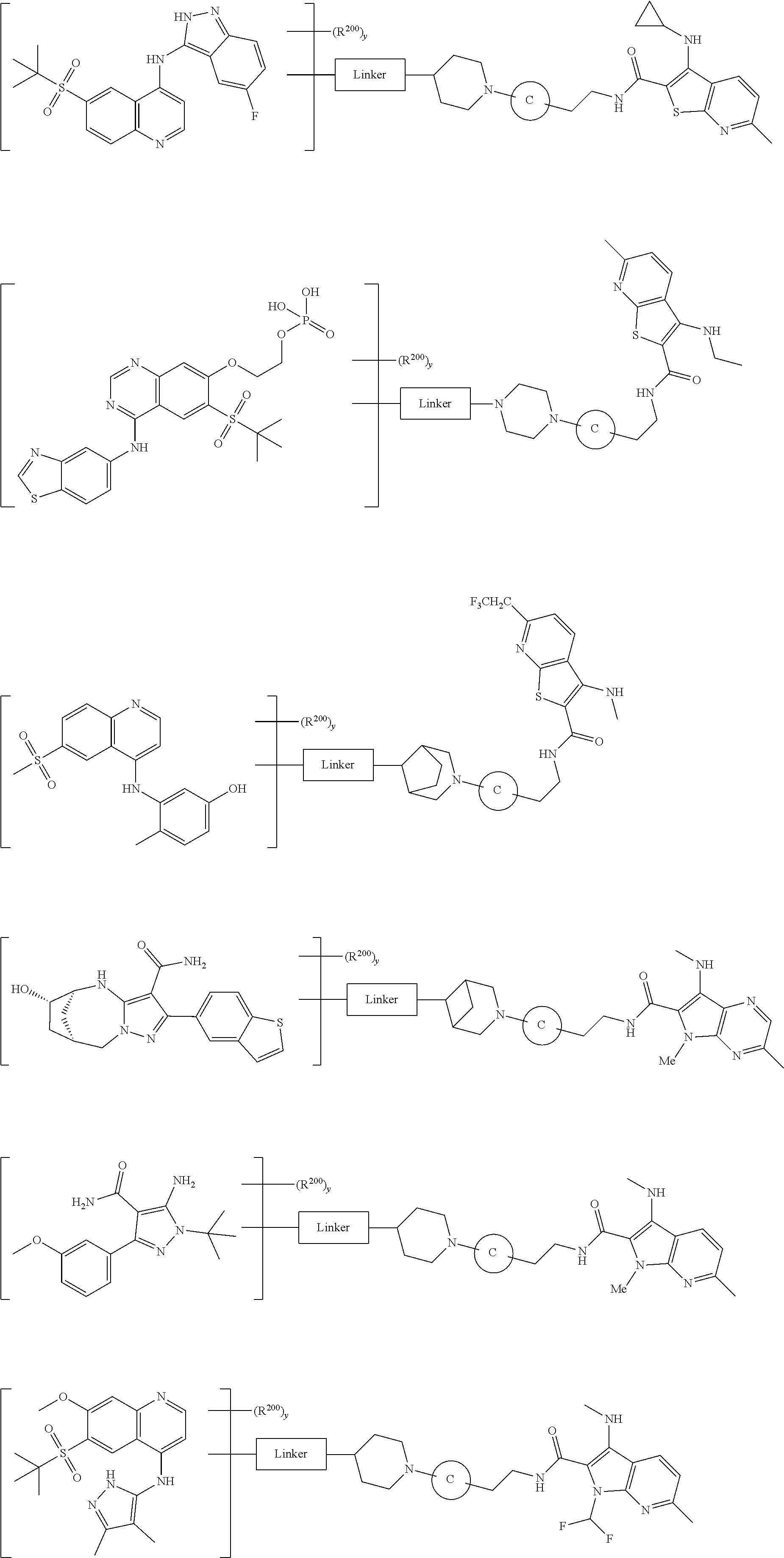

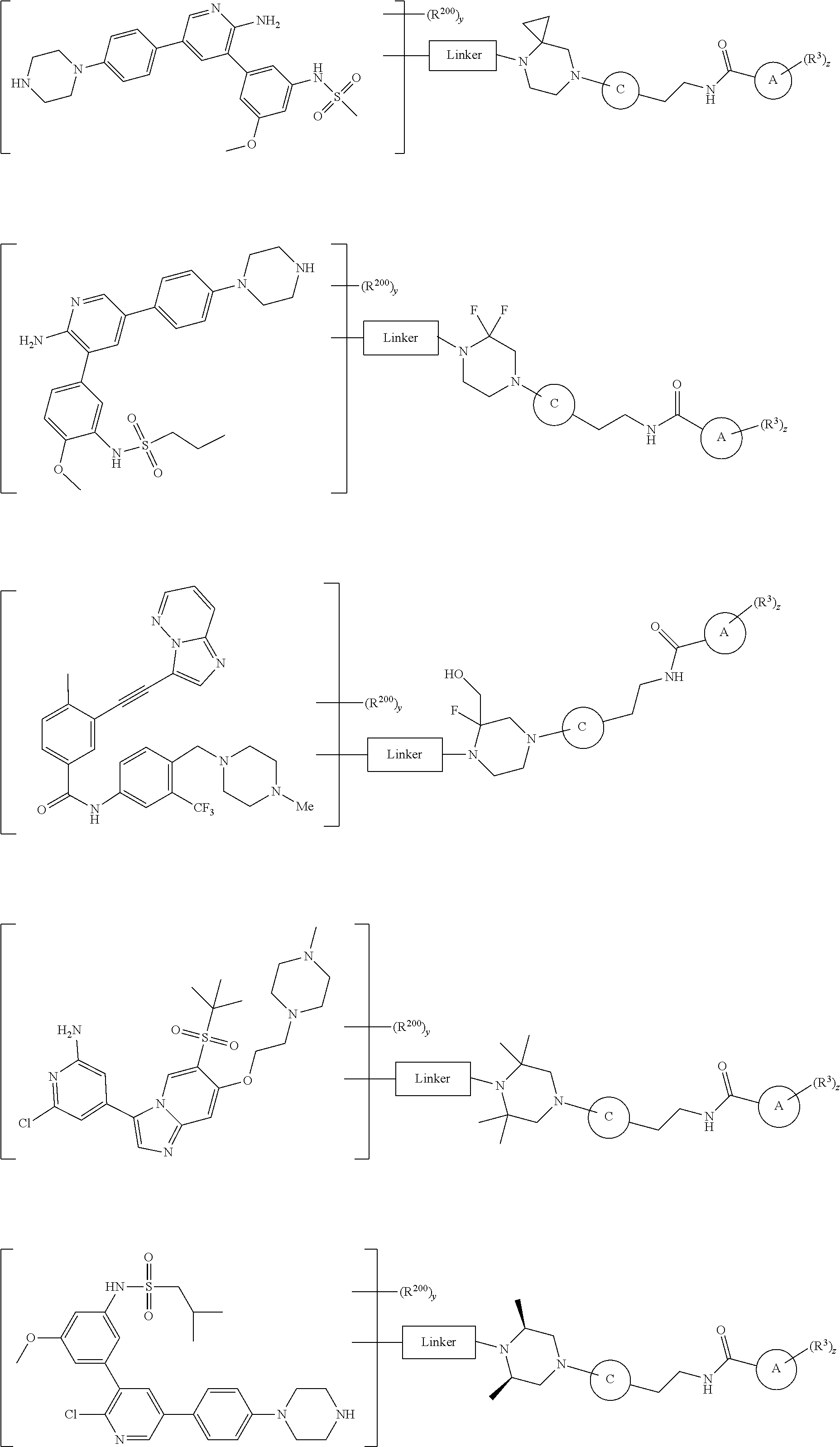

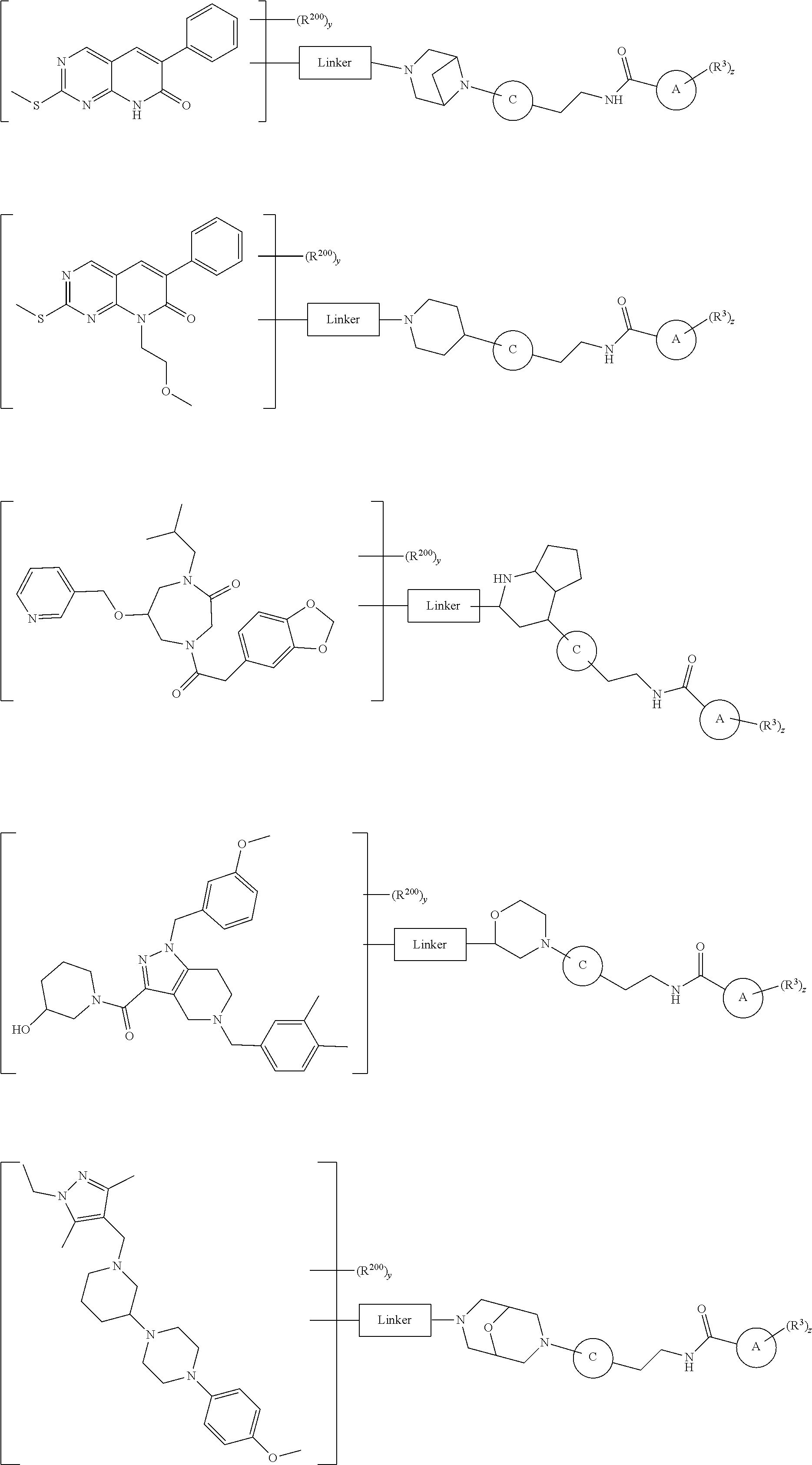

or a pharmaceutically acceptable salt thereof

In certain embodiments the protein stabilizing compound of the present invention is selected from:

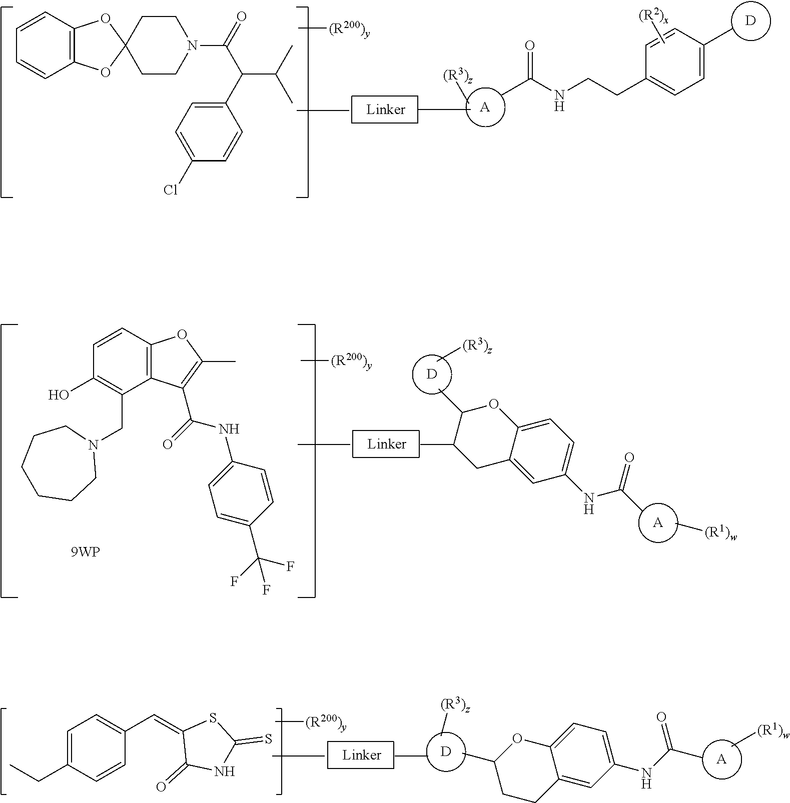

or a pharmaceutically acceptable salt thereof.

In certain embodiments the protein stabilizing compound of the present invention is selected from:

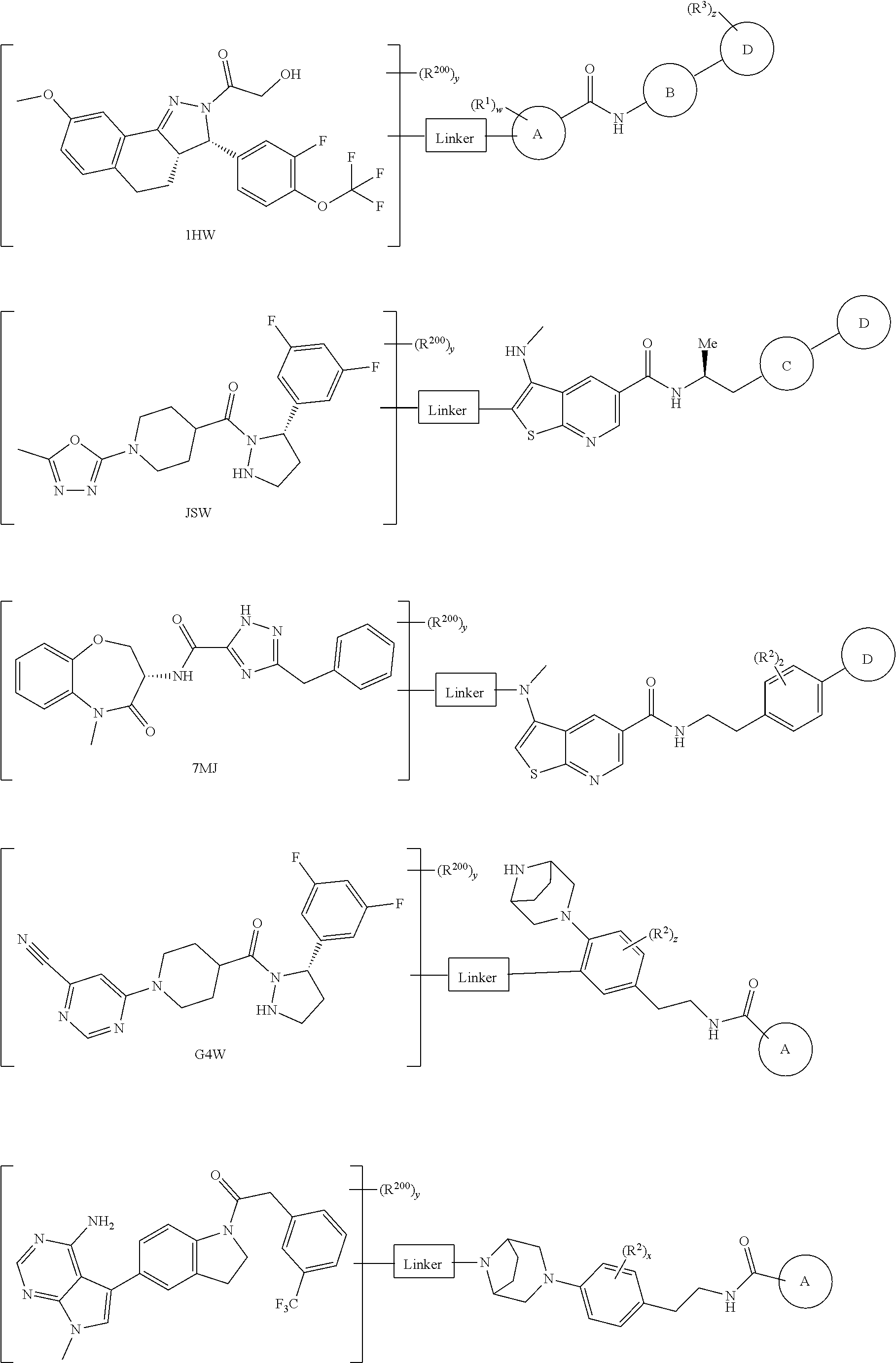

or a pharmaceutically acceptable salt thereof.

In certain embodiments the protein stabilizing compound of the present invention is selected from:

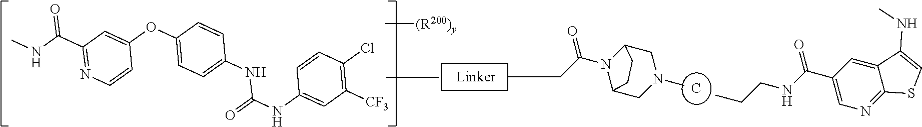

or a pharmaceutically acceptable salt thereof

In certain embodiments the protein stabilizing compound of the present invention is selected from:

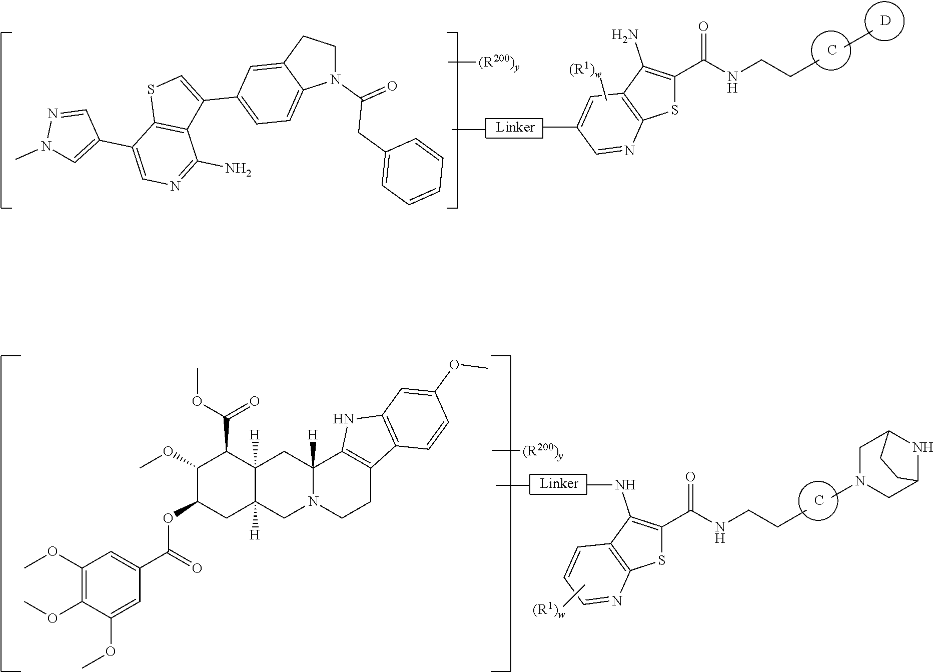

or a pharmaceutically acceptable salt thereof.

In certain embodiments the protein stabilizing compound of the present invention is selected from:

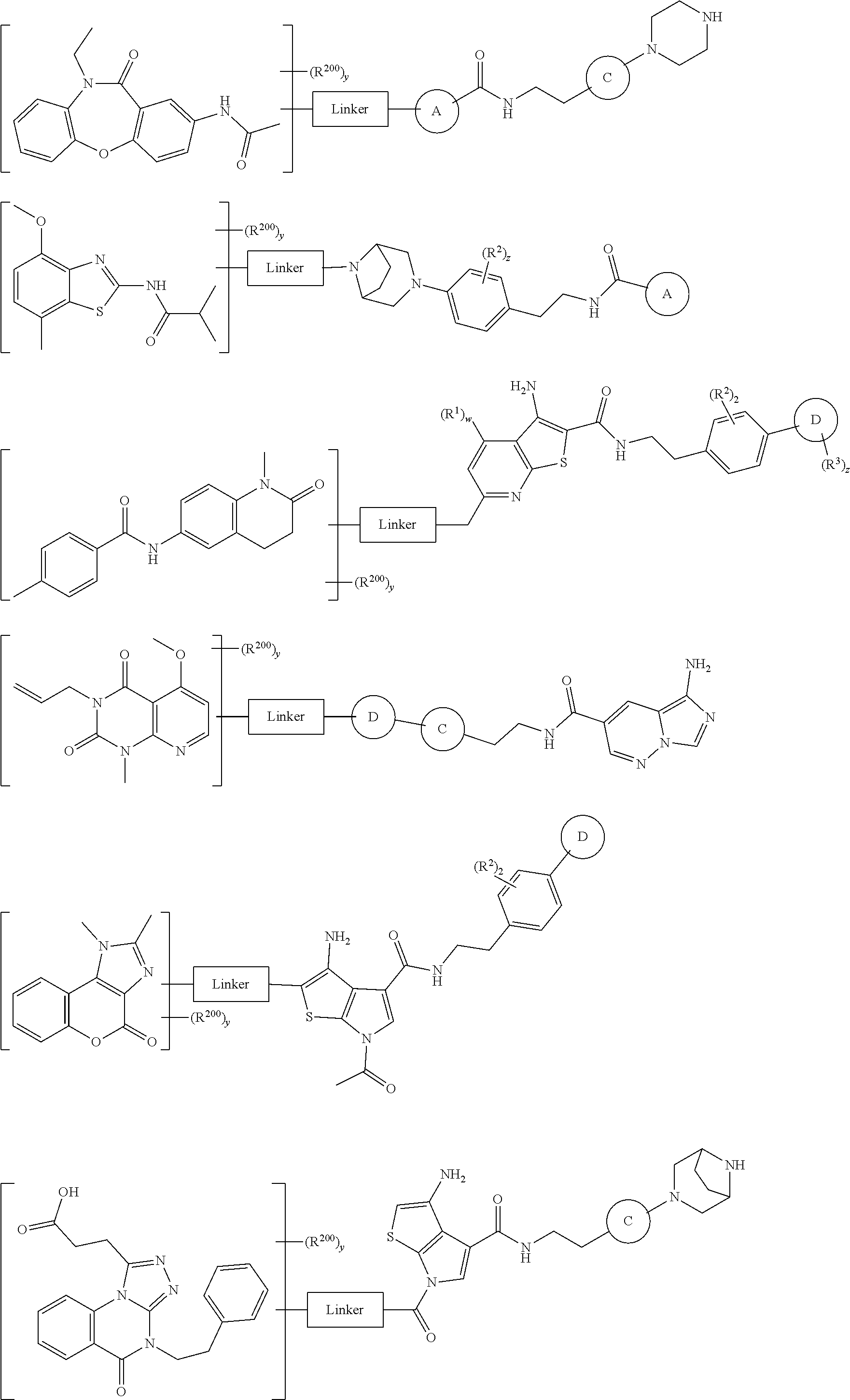

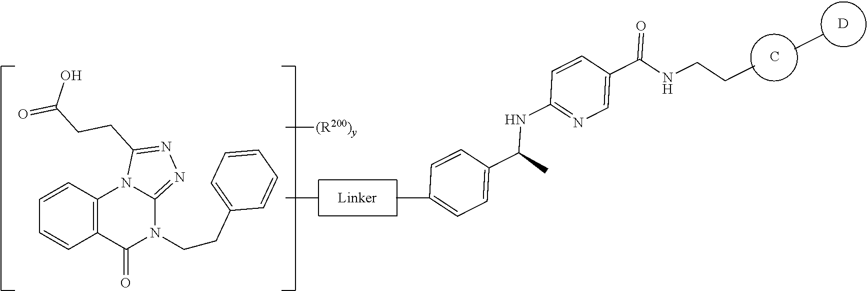

or a pharmaceutically acceptable salt thereof.

In certain embodiments the protein stabilizing compound of the present invention is selected from:

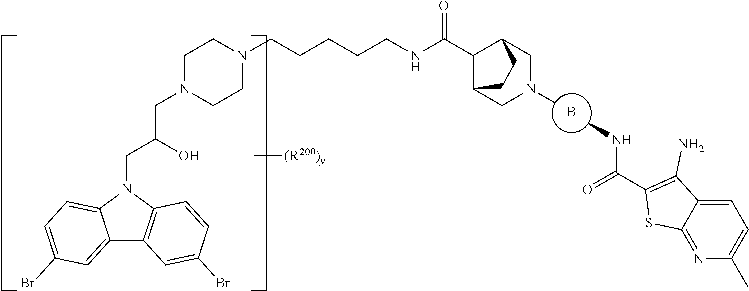

or a pharmaceutically acceptable salt thereof.

In certain embodiments the protein stabilizing compound of the present invention is selected from:

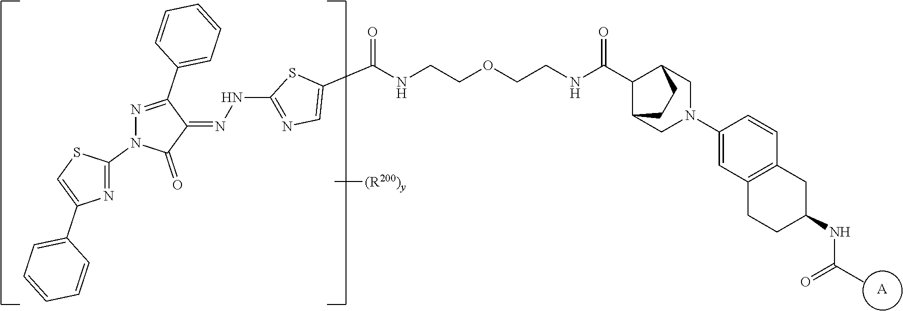

or a pharmaceutically acceptable salt thereof.

In certain embodiments the protein stabilizing compound of the present invention is selected from:

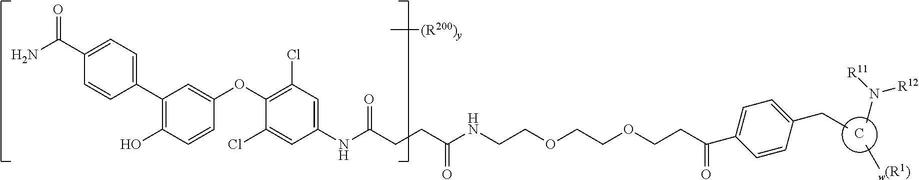

or a pharmaceutically acceptable salt thereof.

In certain embodiments the protein stabilizing compound of the present invention is selected from:

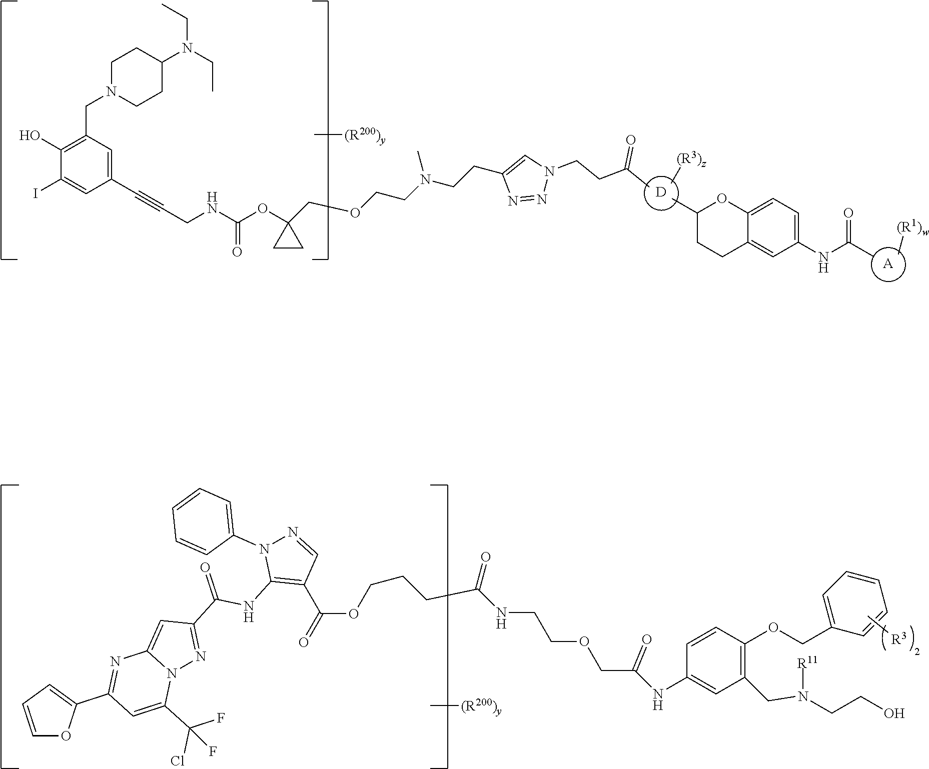

or a pharmaceutically acceptable salt thereof.

In certain embodiments the protein stabilizing compound of the present invention is selected from:

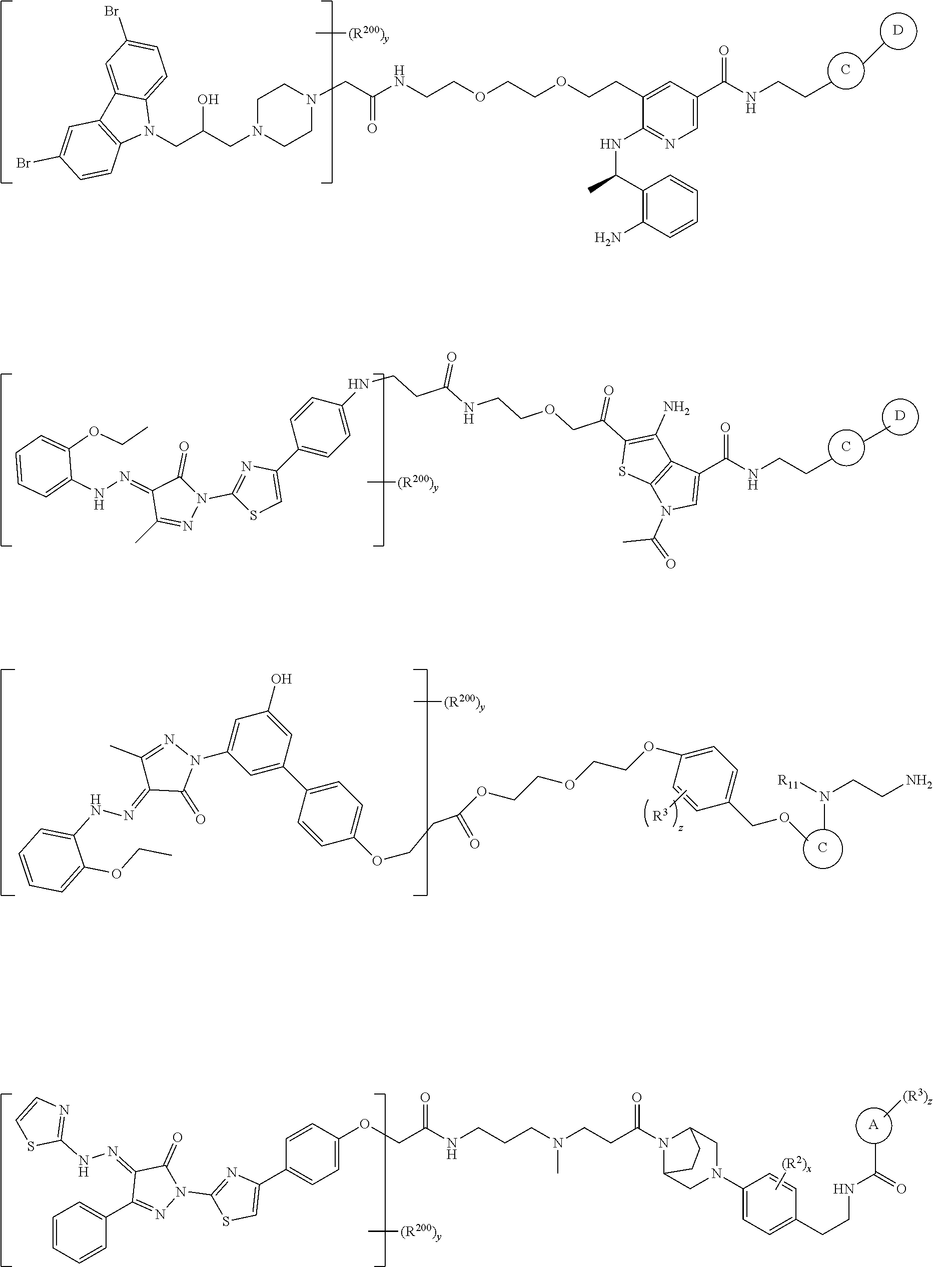

or a pharmaceutically acceptable salt thereof.

In certain embodiments the protein stabilizing compound of the present invention is selected from:

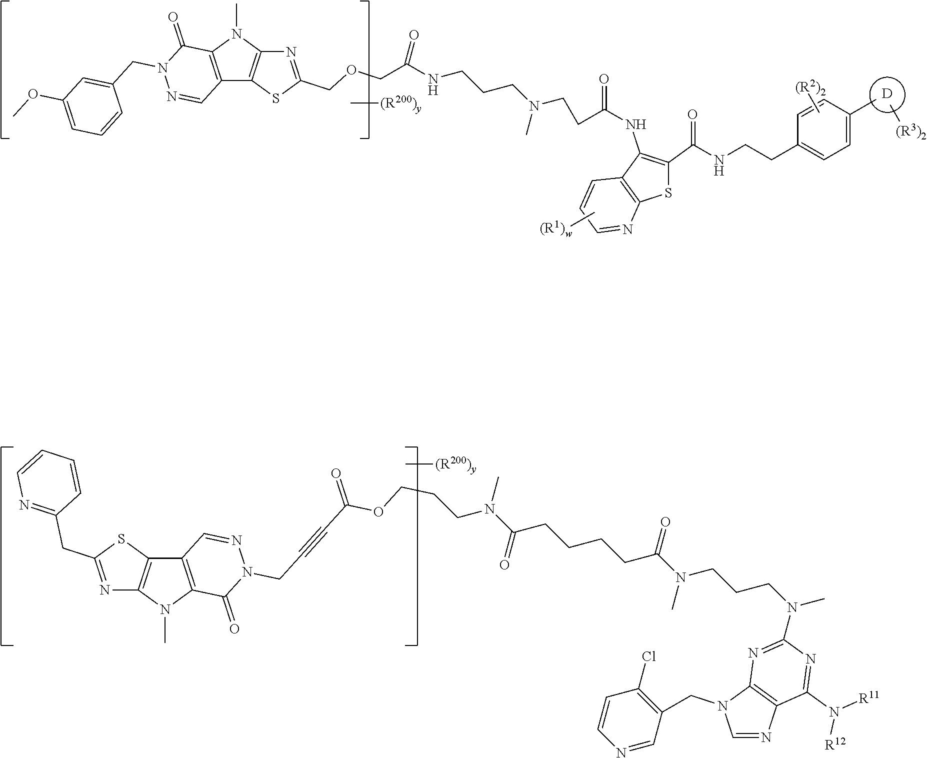

or a pharmaceutically acceptable salt thereof.

In certain embodiments the protein stabilizing compound of the present invention is selected from:

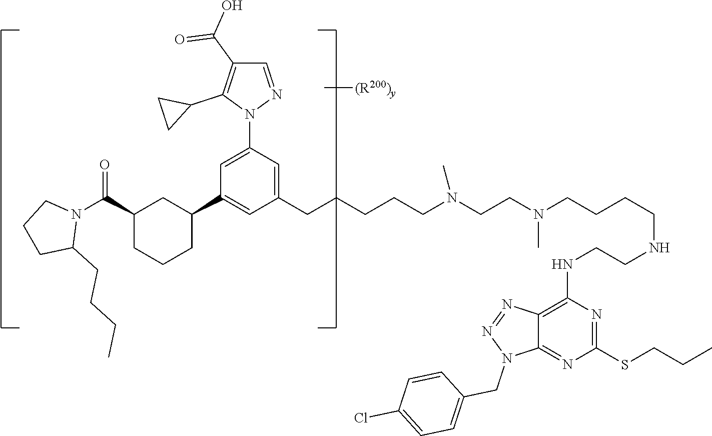

or a pharmaceutically acceptable salt thereof.

In certain embodiments the protein stabilizing compound of the present invention is selected from:

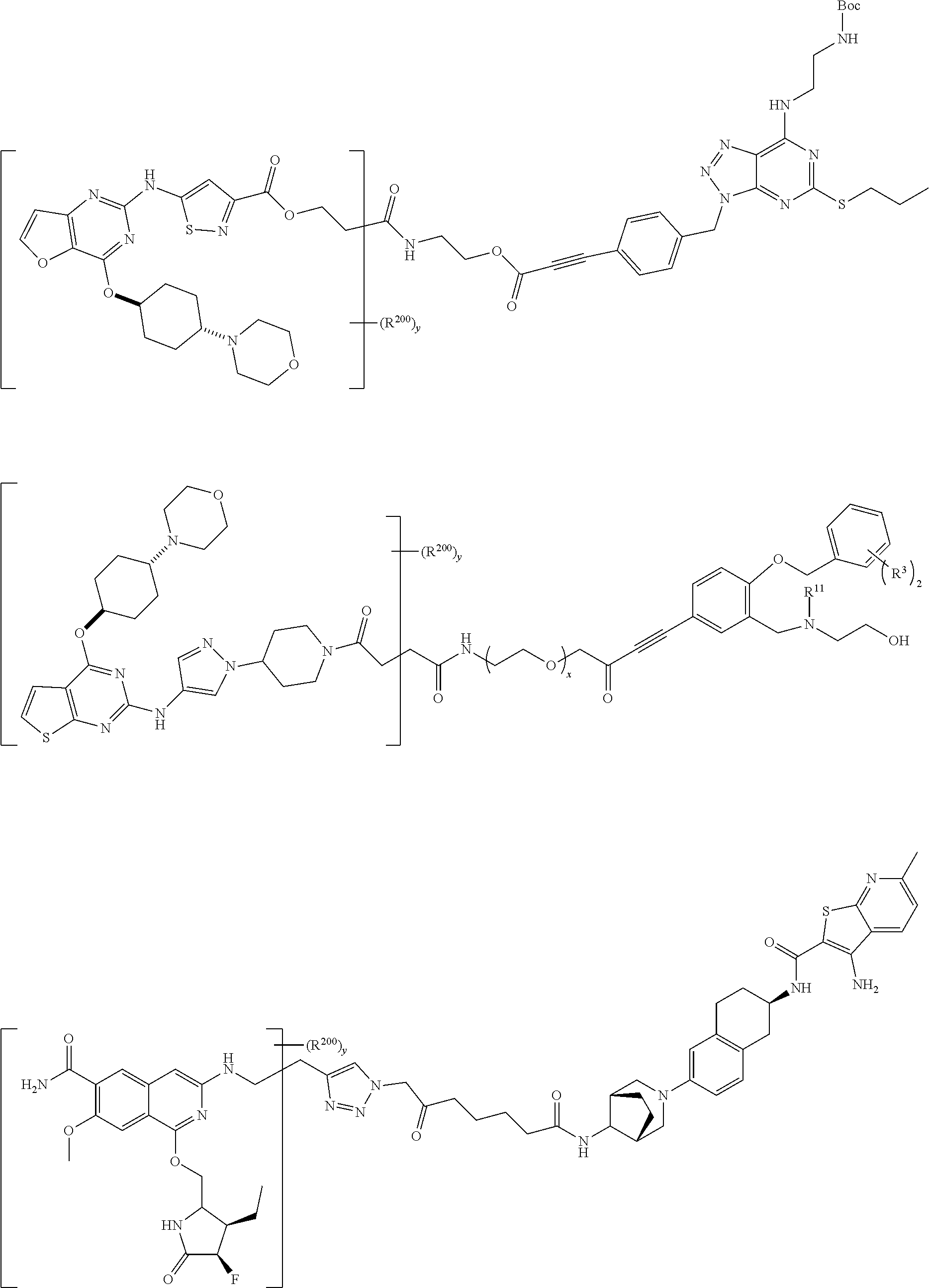

or a pharmaceutically acceptable salt thereof.

In certain embodiments the protein stabilizing compound of the present invention is selected from:

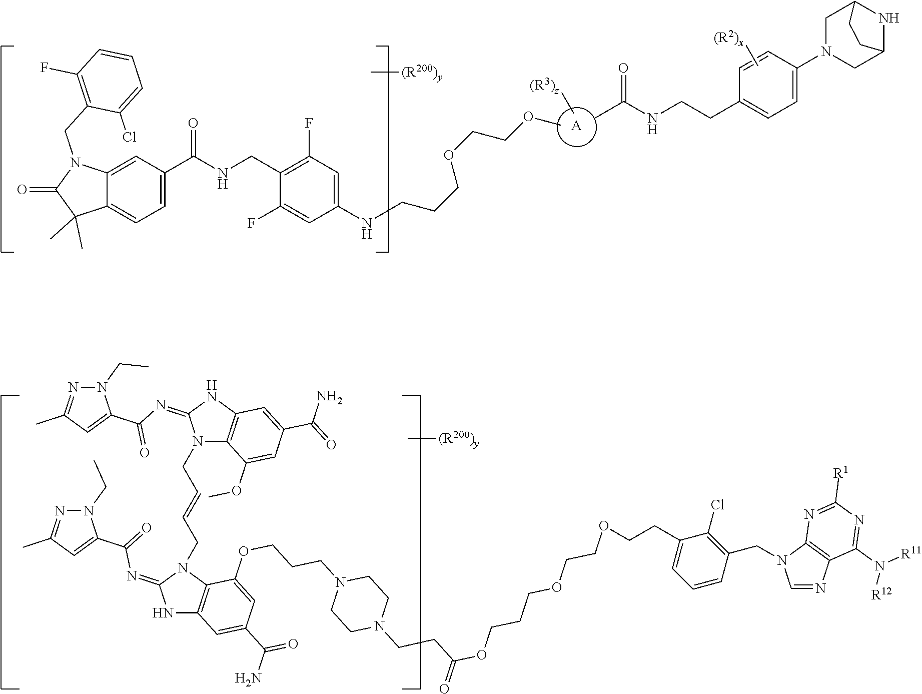

or a pharmaceutically acceptable salt thereof.

In certain embodiments the protein stabilizing compound of the present invention is selected from:

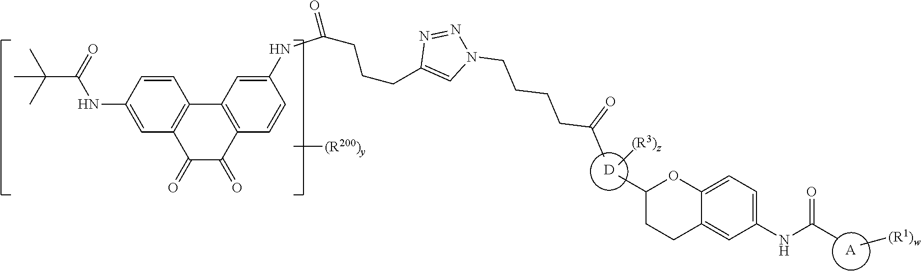

or a pharmaceutically acceptable salt thereof.

In certain embodiments the protein stabilizing compound of the present invention is selected from:

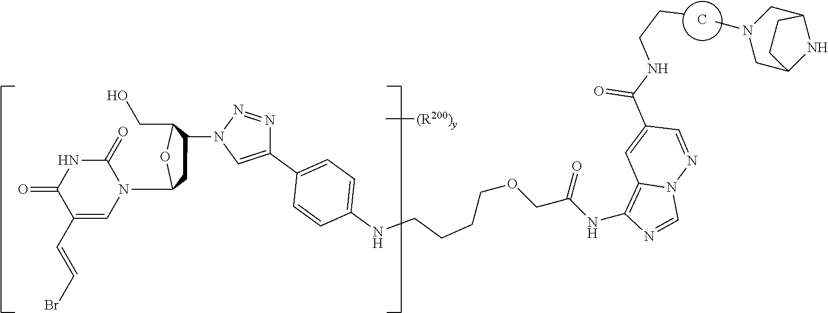

or a pharmaceutically acceptable salt thereof.

In certain embodiments the protein stabilizing compound of the present invention is selected from:

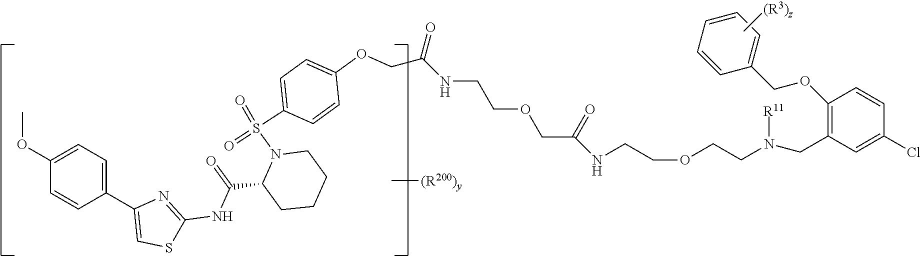

or a pharmaceutically acceptable salt thereof,
wherein:

R⁹⁹ is the attachment point to Linker-Ubiquitinated Protein Targeting Ligand;

R¹⁰⁰ is the attachment point to Linker-USP28 Targeting Ligand; and

R²⁰⁰ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²¹.

### Protein Function Restoration Assays

In certain embodiments a method of stabilizing and restoring a protein's function is provided. The skilled artisan will recognize how to assess whether protein function has been restored in vivo or in vitro depending on context. For example, when the Target Ubiquitinated Protein is an ion channel, such as CFTR, surface representation assays or ion current assays can be used to assay protein function restoration in vitro. Additionally, a reduction of symptoms associated with a disease mediated by the Target Ubiquitinated Protein will show in vivo efficacy. For example, when the Target Ubiquitinated Protein is CFTR amelioration of cystic fibrosis symptoms will result from protein function restoration in vivo. When the Target Ubiquitinated Protein is an oncological target, such as p53, cell death assays or cell cycle assays can be used to demonstrate the restoration of function. When the Target Ubiquitinated Protein is an enzyme then its enzymatic activity can be assayed to demonstrate the restoration of function. Non-limiting examples of these assays are provided below.

### Protein Concentration Assays

The degree of deubiquitination and protein concentration of a protein target of interest in a cell upon treatment with varying concentrations of a compound can be assessed. Briefly, cells that express the target of interest and that have been treated with varying concentrations of compounds will be washed once with PBS without Ca²⁺, harvested, and resuspended in RIPA lysis buffer containing (in mM) Tris (20, pH 7.4), EDTA (1), NaCl (150), 0.1% (wt/vol) SDS, 1% Triton X-100, 1% sodium deoxycholate and supplemented with protease inhibitor mixture (10 μL/mL, Sigma-Aldrich), PMSF (1 mM, Sigma-Aldrich), N-ethylmaleimide (2 mM, Sigma-Aldrich) and PR-619 deubiquitinase inhibitor (50 μM, LifeSensors). Lysates will be prepared by incubation at 4° C. for 1 hr, with occasional vortex, and cleared by centrifugation (10,000×g, 10 min, 4° C.). Supernatants will be transferred to new tubes, with aliquots removed for quantification of total protein concentration determined by the bis-cinchonic acid protein estimation kit (Pierce Technologies). Lysates will be pre-cleared by incubation with 10 μL Protein A/G Sepharose beads (Rockland) for 40 min at 4° C. and then incubated with 0.75 μg anti-Q1 antibody (Alomone) for 1 hr at 4° C. Equivalent total protein amounts will be added to spin-columns containing 25 μL Protein A/G Sepharose beads, tumbling overnight at 4° C. Equivalent total protein amounts of pre-cleared lysates for the target of interest pulldowns will be added directly to 20 μL RFP-Trap conjugated agarose beads (Chromotek, rta-20), tumbling overnight at 4° C. Immunoprecipitates will be washed twice with RIPA buffer, 3 times with high salt RIPA (500 mM NaCl), spun down at 500× g, and eluted with 40 μL of warmed sample buffer [50 mM Tris, 10% (vol/vol) glycerol, 2% SDS, 100 mM DTT, and 0.2 mg/mL bromophenol blue], and boiled (55° C., 15 min). Proteins will be resolved on a 4-12% Bis Tris gradient precast gel (Life Technologies) in Mops-SDS running buffer (Life Technologies) at 200 V constant for ˜1 h. Protein bands will be transferred by tank transfer onto a nitrocellulose membrane in transfer buffer (25 mM Tris pH 8.3, 192 mM glycine, 15% (vol/vol) methanol, and 0.1% SDS). The membranes will be blocked with a solution of 5% nonfat milk in tris-buffered saline-tween (TBS-T) (25 mM Tris pH 7.4, 150 mM NaCl, and 0.1% Tween-20) for 1 hr at RT and then incubated overnight at 4° C. with primary antibodies against the target of interest in blocking solution. The blots will be washed with TBS-T three times for 10 min each and then incubated with secondary horseradish peroxidase-conjugated antibody for 1 hr at RT. After washing in TBS-T, the blots will be developed with a chemiluminiscent detection kit (Pierce Technologies) and then visualized on a gel imager. Membranes can then be stripped with harsh stripping buffer (2% SDS, 62 mM Tris pH 6.8, 0.8% 13-mercaptoethanol) at 50° C. for 30 min, rinsed under running water for 2 min, and washed with TBST (3×, 10 min). Membranes can then be pre-treated with 0.5% glutaraldehyde and re-blotted with an anti-ubiquitin antibody (LifeSensors VU1, 1:500) to assess the effect of treatment on the amount of ubiquitin present on the target.

Additional methods that can be used to determine the concentration of a target protein after administration of a compound of the invention include but are not limited to LC-MS/MS, Bradford assay, BCA assay.

Protein Stabilization Assays-HiBiT Assay

### I. Cell Line Overview

HiBiT Stable Cell Lines are generated by using site-specific insertion via CRISPR-Cas9 to fuse the 11-amino-acid HiBiT peptide tag to either the N‘ or C’ terminus of the protein of interest (POI) depending on factors such as success of tagged POI expression or tag location (intracellular vs. extracellular side of a membrane protein). POI may include but are not limited to intracellular or intramembrane proteins. In the case of heterologous cells (i.e. HEK293), the HiBiT Stable Cell Line may also stably express intracellular NanoLuc luciferase-based LgBiT protein. The HiBiT and LgBiT proteins, when combined, reconstitute the active NanoBiT luciferase enzyme, which emits a luminescent signal in the presence of substrate (i.e. Nano-Glo Live Cell furimazine-based substrates). Stable Cells may stably express the HiBiT protein as a pool of cells or as a single clone (heterozygous or homozygous expression depending on target).

Ii. HiBiT Kinetic Assay Protocol to Determine Protein Stabilization

The following protocol describes a high throughput assay capable of screening multiple compounds at several doses on a HiBiT-tagged POI.
1. HiBiT cell lines are plated up to 1 day prior to the assay in a tissue-culture-treated white 96 well plate with a lid using 100 μl DMEM+8% FBS+1% penicillin/streptomycin/glutamine media/well at a cell density of 5-20k cells/well.
2. The following day, cells are equilibrated for 2.5 hours with lx Nano-Glo Endurazine Live Cell substrate (50p/well) in C₀₂ independent media+8% FBS+1% penicillin/streptomycin/glutamine to generate a stable background luminescent signal.
3. Cycloheximide is added at 2× concentration (i.e. 200 μM) in 50 μL/well to achieve a final 100 μM per well. For dose response measurement of compounds, suitable stock solutions are prepared at desired concentrations and are added concomitantly with the cycloheximide treatment.
4. Well Plates with cells are immediately moved to a plate reader capable of measuring luminescence with temperature set at 37° C. (e.g. Promega Glomax).
5. Luminescence signal is measured at 1-3 time points*optimized to the POI to observe differences in protein levels. At the final time point, cells are assessed for compound toxicity via CellTiter-Glo (see separate protocol).
6. Raw Data is converted to fold change over DMSO control at the specific time point and normalized with cell viability data to account for protein levels that may change with cell viability.
7. Compounds are selected for a secondary screen if protein levels from co-treatment with cycloheximide are significantly higher than that of with cycloheximide-only treatment.
8. Cells treated with compound in a secondary screen (follow Protocol item 1-4) are assessed over a continuous time course as the cells are incubated in compound, with an integration time of 0.5-2 seconds every 1-2 hrs for 24-72 hrs (depending on half-life of assayed POI).
9. Raw Data is converted to fold change over DMSO control at the specific time point and plotted as a one phase decay plot. Half life calculations of the POI are determined based on the decay plot and compared between cycloheximide alone (steady-state POI degradation) cell treatment and cell treatment with cycloheximide plus the compound. componds that significantly extend the half-life of the POI are considered to stabilize the POI by deubiquitination from the recruited DUB.
NOTE: optimization of this time point is based on running a continuous 24-72 hr kinetic assay on the POI using cycloheximide, which generates data on protein half life. Each new target may be assessed initially in a cycloheximide chase screen before running the screen.

### Ion Channel Function Assays

Cell surface and total ion channel pools will be assayed by flow cytometry in live, transfected HEK293 cells that are treated with varying concentrations of compounds. 48 hrs post-transfection, cells cultured in 12-well plates will be gently washed with ice cold PBS containing Ca²⁺ and Mg²⁺ (in mM: 0.9 CaCl₂, 0.49 MgCl₂, pH 7.4), and incubated for 30 min in blocking medium (DMEM with 3% BSA) at 4° C. HEK293 cells expressing the ion channel of import will then be incubated with 1 μM Alexa Fluor 647 conjugated α-bungarotoxin (BTX₆₄₇; Life Technologies) in DMEM13% BSA on a rocker at 4° C. for 1 hr, followed by washing three times with PBS (containing Ca²⁺ and Mg²⁺). Cells will be harvested in Ca²⁺-free PBS, and assayed by flow cytometry. CFP—and YFP-tagged proteins are excited at 405 and 488 nm, respectively, and Alexa Fluor 647 is excited at 633 nm. The amount of ion channel at the surface (strength of fluorescent signal with Alexa Fluor 647) will be compared across the cell samples treated with differing amounts of compound.

To measure the functional restoration of ion channels upon treatment, electrophysiology experiments will be performed. For potassium channel measurements, whole-cell membrane currents will be recorded at room temperature in CHO cells using a patch-clamp amplifier. A coverslip with adherent CHO cells will be placed on the glass bottom of a recording chamber (0.7-1 mL in volume) mounted on the stage of an inverted microscope. An internal solution containing (mM): 133 KCl, 0.4 GTP, 10 EGTA, 1 MgSO₄, 5 K₂ATP, 0.5 CaCl₂, and 10 HEPES (pH 7.2) and an external solution containing (in mM): 147 NaCl, 4 KCl, 2 CaCl₂, and 10 HEPES (pH 7.4) will be used. Pipette resistance will be typically 1.5 MΩ when filled with the internal solution. I-V curves will be generated from a family of step depolarizations (−40 to +100 mV in 10 mV steps from a holding potential of −80 mV). Currents will be sampled at 20 kHz and filtered at 5 kHz. Traces will be acquired at a repetition interval of 10 s.

For whole-cell recordings of cardiomyocytes (KCQN1 target), they will be performed 48-72 hrs after expression of the channel and treatment with the compounds. The same internal and external solutions as are being used above will be used for the experiments. A slow voltage ramp protocol (from −80 my to +100 mV over 2 s) will be used to evoke whole-cell currents. Action potential recordings under current clamp will be obtained via 0.25 Hz stimulation with short current pulses (150 pA. 10 ms).

For CFTR channel measurements, whole-cell recordings will be carried out in HEK293 and FRT cells at room temperature. An internal solution containing (mM): 113 L-aspartic acid, 113 CsOH, 27 CsCl, 1 NaCl, 1 MgCl₂, 1 EGTA, 10 TES, 3 MgATP (pH 7.2) and an external solution containing (in mM): 145 NaCl, 4 CsCl, 1 CaCl₂, 1 MgCl₂, 10 glucose, and 10 TES (pH 7.4) will be used for the experiments. I-V curves will be generated from a family of step depolarizations (−80 to +80 mV in 20 mV steps from a holding potential of −40 mV). CFTR currents are activated by perfusion with 10 μM forskolin. In experiments utilizing VX809 (3 μM) (as a positive control), the drug will be added for 24 hrs post-transfection and incubated at 37° C. VX770 (positive control) will be used acutely at 5 μM concentration. For experiments using compounds, multiple concentrations will be tried. Currents will be sampled at 20 kHz and filtered at 7 kHz. Traces will be acquired at a repetition interval of 10 sec.

### Cell Death Assays

A luciferase-based assay reaction will be used to assess cell viability. This assay can be used to determine the effects on cell viability with differing treatments of a test agent. The assay format results in cell lysis and generation of a luminescent signal that is proportional to the amount of ATP present. The amount of ATP is directly proportional to the number of live cells present in a test sample. Briefly, in opaque-walled multiwell plates mammalian cells will be plated at a density of 20k/well in culture medium. Prepare control wells containing medium without cells to determine background signal. After 24 hrs. add compounds to experimental wells and incubate for another 24 hrs. Equilibrate the plate and its contents to room temperature for approximately 30 minutes. Add 100 μL of pre-equilibrated test reagent volume (i.e. CellTiter-Glo® 2.0 Reagent) to each well equal to the volume of cell culture medium present in each well. Mix the contents for 2 minutes on an orbital shaker to induce cell lysis on a plate shaker at 500-700 rpm. Record luminescence using an integration time of 0.25-1 second per well as a guideline. The brighter the luminescent signal the more live cells you have in the sample. Viability curves versus amount of compound added can be analyzed to assess the effect of a compound on the restoration of a target of interest that results in increased cell viability.

### Cell Cycle Assays

The ability of a stabilizing compound described herein to restore the function of a protein such as a tumor suppressor can result in the cell persisting in a particular phase of the cell cycle leading to prolonging of the cell cycle and ultimately programmed cell death. The cell cycle stage at which a population of cells exists can be determined by analyzing the DNA content and distribution of the cellular DNA using flow cytometry. The assays described in Gray et al., “Cell cycle analysis using flow cytometry” International Journal of Radiation Biology and Related Studies in Physics, Chemistry and Medicine 1986, (49:2), 237-255, can be used to determine which phase of the cell cycle a cell population is in and allow for the monitoring of cell cycle changes as populations of cells are perturbed in the presence or absence of a test article.

### Enzymatic Activity Assays

Enzymatic assays will be run on targets that are enzymes such as phenylalanine hydroxylase, (PAH). Patient derived primary cells or stable cell-lines (i.e. HEK293) expressing wild type or clinically relevant mutations of PAH (i.e. R261Q or Y414C) will be used for further study. These cells will be treated with various concentrations of compounds to quantify their restorative effect. Cells will be harvested and lysed using 3× freeze-thaw cycles in Tris-KCL (0.03 uM Tris, 0.2M KCL, pH7.2) lysis buffer containing protease inhibitors. Cell lysates will be clarified for 20 min centrifugation at 3000 rcf at 4° C. The lysates will be used for activity assays. 20 ul of lysate will be incubated with 1M phenylalanine and 1 mg/ml catalase for 5 min at room temperature in 15 mM HEPES pH 7.3 followed by 1 min incubation with 10 uM ferrous ammonium sulfate. The reaction will be initiated by addition of 75 uM BH₄ stabilized in 2 mM DTT for 60 min at 25° C. and stopped by acetic acid followed by 10 min incubation at 95° C. Total reaction volume is 100 ul. The amount of tyrosine production will be measured and quantified by HPLC. The more amount of tyrosine produced will correlate with increased amounts of the PAH enzyme produced and stabilized as a function of cell treatment with a compound.

### Immunology and Immuno-Oncology Assays (Part 1)

Assays to monitor cytokine expression and release upon cell treatment with a compound will be run. To monitor the gene expression of a cytokine it is possible to use a real time RT-PCR approach. Briefly, purify cellular RNA from cells that are both treated (experimental set) and untreated (control) with compounds. Using at least 10⁶ cells aspirate media and wash with ice cold PBS. Aspirate PBS and add 1 ml TRizol. Scrape the plate and transfer the TRizol/cell lysate into an 1.5 ml tube. Leave at RT for 5 min. Add 250 ul of chloroform and shake tube vigorously for 15 sec. Leave at RT for 5 min and then centrifuge sample at 10k for 5 min. The resultant mixture will have three phases; remove the top phase (aqueous) and place in another tube. Add 550 ul of isopropanol to the aqueous phase and mix gently. Let sit at RT for 5 min. Centrifuge at 14k rpm for 30 min. Place samples on ice. Pour off isopropanol and wash pellet with 75% ethanol. Recentrifuge at 9.5K rpm for 5 min. Resuspend the pellet in 25 μL of water. The resulting RNA prep should have a 260/280 ratio of >1.8. The purified RNA can now be used to create cDNA. Briefly, prepare the following reaction tube with 5 ug total RNA, 3 ul random hexamer primers (50 ng/ul), 10 mM dNTP, and bring up to 10 ul with water. Incubate the samples at 65° C. for 5 min and then on ice for at least 1 min. For each reaction add 4 ul of 25 mM MgCl₂, 1M DTT, and RNAase inhibitor, mix briefly, and then place at room temperature for 2 min. Add 50 units of reverse transcriptase to each reaction, mix and incubate at 25° C. for 10 min. Incubate the reactions at 42° C. for 50 min, heat inactivate at 70° C. for 15 min, and then chill on ice. Add 1 μl RNase H and incubate at 37° C. for 20 min. Store the cDNA at −20° C. for use in the real-time PCR experiment.

For Real time PCR design primers specific for the cytokine gene of interest you are looking to analyze the change in expression upon treatment. For each gene-specific forward and reverse primer pair add 2 μL of a 5 pmol/ul stock, 0.5 ul cDNA (5 ng total), 25 ul SYBR green mix, 22.5 ul water.

Run the PCR reaction in a Real Time PCR machine with the following extension times:
1. 50° C. 2 min, 1 cycle
2. 95° C. 10 min, 1 cycle
3. 95° C. 15 s->60° C. 30 s->72° C. 30 s, 40 cycles
4. 72° C. 10 min, 1 cycle

After the PCR is finished perform a dissociation curve analysis comparing the treated samples to the untreated control set. A decrease of the cycle time for amplification of a particular cytokine gene under an experimental condition (treatment) suggests that restoration of a target of interest has led to an increase in the gene expression of a particular cytokine.

In addition to looking at cytokine expression at the transcriptional level, it is possible to analyze cytokine protein expression levels that are either secreted or produced internally in cells that are treated with varying amounts of compounds. The use of cytokine arrays has the advantage of looking at multiple cytokines at once. Briefly, seed plates and transfer media to low-serum medium (<0.2% calf serum). Treat cells with varying amounts of compounds (experimental). After 24 hrs. Collect the conditioned media. Spin at 1000g at 4° C. for 10 min. Remove supernatant and freeze until use. Use protein concentration of cell lysate to normalize the protein amounts for the array. The cytokine array procedure is based on the sandwich ELISA technique. Commercially available membranes with immobilized antibodies to the cytokines of interest will be used. Block the membranes with bovine serum albumin for 30 min at room temperature. Incubate the membrane with sample conditioned media at room temperature for 1-2 hr. Wash membranes with TBS/Tween-20. Incubate membranes with biotin-labeled secondary antibodies at room temperature for 1-2 hours. Wash membrane with TBS/Tween-20. Incubate membranes with Horseradish peroxidate-streptavidn (HRP) at room temperature for 1 hr. Wash membranes, add HRP substrate, and visualize signal. Wells that light up are indicative of the presence of a particular cytokine secreted into the conditioned media. Comparing the signals between the test sample and the controls will allow determination of cytokine production in response to treatment.

### Immunology and Immuno-Oncology Assays (Part 2)

In vitro assays to analyze the effect of compounds on T-cell function will be run. For example a luciferase based assay to determine T-cell proliferation in response to treatment will be run that is similar to the viability assay described above in the Cell Death Assays. Briefly human primary blood mononuclear cells will be seeded and treated with varying concentrations of compounds. The population of cells will then be stimulated with anti-CD28 and anti-CD03 antibodies (10 ug/ml) and the cell proliferation measured 2-day and 5-days post treatment. Cell proliferation will be measured using the amount of ATP as a surrogate for live cell proliferation (i.e. CellTiter-Glo® 2.0 Reagent). Differences in cell number between treated samples and untreated samples will be assessed for restoration of target function and their subsequent effect on T-cell proliferation.

### Processes of Manufacture:

The protein stabilizing compound of the present invention can be manufactured according to routes described in the Working Examples below or as otherwise known in the patent or scientific literature and if appropriate supported by the knowledge of the ordinary worker or common general knowledge.

Some of the carbons in the compounds described herein are drawn with designated stereochemistry. Other carbons are drawn without stereochemical designation. When drawn without designated stereochemistry, that carbon can be in any desired stereochemical configuration that achieves the desired purpose. One skilled in the art will recognize that pure enantiomers, enantiomerically enriched compounds, racemates and diastereomers can be prepared by methods known in the art as guided by the information provided herein. Examples of methods to obtain optically active materials include at least the following:
i) chiral liquid chromatography—a technique whereby diastereomers are separated in a liquid mobile phase by virtue of their differing interactions with a stationary phase (including vial chiral HPLC). The stationary phase can be made of chiral material or the mobile phase can contain an additional chiral material to provoke the differing interactions;
ii) non-chiral chromatography of diastereomers—often diastereomers can be separated using normal non-chiral column conditions;
iii) chiral gas chromatography—a technique whereby the racemate is volatilized and enantiomers are separated by virtue of their differing interactions in the gaseous mobile phase with a column containing a fixed non-racemic chiral adsorbent phase;
iv) simultaneous crystallization—a technique whereby the individual diastereomers are separately crystallized from a solution;
v) enzymatic resolutions—a technique whereby partial or complete separation of diastereomers are separated by virtue of differing rates of reaction with an enzyme;
vi) chemical asymmetric synthesis—a synthetic technique whereby the desired diastereomer is synthesized from an achiral precursor under conditions that produce asymmetry (i.e. chirality) in the product, which may be achieved by chiral catalysts or chiral auxiliaries;
vii) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences the chiral auxiliary later removed to obtain the desired enantiomer; and
viii) extraction with chiral solvents—a technique whereby diastereomers are separated by virtue of preferential dissolution of one over the others in a particular chiral solvent.

TABLE 1
Abbreviations table

| Abbrev. | Name |
|---|---|
| 4-DMAP | 4-Dimethylaminopyridine |
| AcCN | acetyl cyanide |
| ACN | acetonitrile |
| AcOH, HOAc | acetic acid |
| AcONa, | sodium acetate, |
| NaOAc | |
| B₂Pin₂ | Bis(pinacolato)diboron |
| Boc₂O | Di-tert-butyl dicarbonate |
| BOP-Cl | Bis(2-oxo-3-oxazolidinyl)phosphinic chloride |
| Br₂ | bromine gas |
| BuOH | butanol |
| CbzCl | Benzyl chloroformate, |
| ClCH₂COCl | chloroacetyl chloride |
| Cs₂CO₃ | cesium carbonate |
| CuI | copper (I) iodide |
| CuSO₄ | copper (II) sulfate |
| DCE | 1,2-dichloroethane |
| DCM | dichloromethane |
| DIAD | Diisopropyl azodicarboxylate |
| DIEA, DIPEA | N,N-Diisopropylethylamine |
| DMF | Dimethylformamide |
| DMSO | dimethylsulfoxide |
| DPPA | Diphenylphosphoryl azide |
| DTT | Dithiothreitol |
| EA | ethyl acetate |
| EDCI | 1-Ethyl-3-(3-dimethylaminopropyl)carbodiimide |
| Et₃N, TEA | triethylamine |
| Et₃SiH | Triethylsilane |
| EtOH | ethanol |
| FA, HCO₂H | formic acid |
| Fe | iron |
| H₂ | hydrogen gas |
| H₂O | water |
| H₂SO₄ | sulfuric acid |
| HATU | 1-[Bis(dimethylamino)methylene]-1H-1,2,3- |
| | triazolo[4,5-b]pyridinium 3-oxide |
| | hexafluorophosphate, |
| HCl | hydrochloric acid |
| HOAt | 1-Hydroxy-7-azabenzotriazole |
| HOBt | Hydroxybenzotriazole |
| HPLC | high performance liquid chromatography |
| K₂CO₃ | potassium carbonate |
| K₃PO₄ | potassium phosphate |
| KI | potassium iodide |
| KOAc | potassium acetate |
| KSCOCH₃ | Potassium thioacetate |
| LCMS | liquid chromatography mass spectrometry |
| m-CPBA | meta-Chloroperoxybenzoic acid |
| MeMgBr | Methylmagnesium bromide |
| MeOH | methanol |
| MsCl | Methanesulfonyl chloride |
| MTBE | methyl tert-butyl ether |
| N₂ | nitrogen gas |
| Na₂CO₃ | sodium carbonate |
| Na₂SO₃ | sodium sulfite |
| Na₂SO₄ | sodium sulfate |
| NaBH₃CN | Sodium cyanoborohydride |
| NaBH₄ | sodium borohydride |
| NaCl | sodium chloride |
| NaClO | sodium chlorite |
| NaH | sodium hydride |
| NaHCO₃ | sodium hydrogen carbonate |
| NaOH | sodium hydroxide |
| NBS | N-Bromosuccinimide |
| n-BuLi | n-Butyllithium |
| n-BuOH | butyl alcohol (1-butanol) |
| NH₂Boc, | tert-Butyl carbamate; Boc-amide |
| BocNH₂ | |
| NH₃ | ammonia |
| NH₃H₂O | ammonia solution |
| NH₄Cl | ammonium chloride |
| NHMeOMe | N,O-Dimethylhydroxylamine hydrochloride |
| NIS | N-Iodosuccinimide |
| NMM | N-Methylmorpholine |
| Pd(dppf)Cl₂ | 1,1′- |
| | Bis(diphenylphosphino)ferrocenedichloropalladium(II) |
| Pd(dtbpf)Cl₂ | [1,1′-Bis(di-tert- |
| | butylphosphino)ferrocene]dichloropalladium(II) |
| Pd(OAc)₂ | Palladium(II) acetate |
| Pd(OH)₂ | Palladium hydroxide |
| Pd(PPh₃)₂Cl₂, | Bis(triphenylphosphine)palladium chloride |
| Pd(Ph₃P)₂Cl₂ | |
| Pd(PPh₃)₄ | Tetrakis(triphenylphosphine)palladium(0) |
| Pd/C | palladium carbon |
| Pd- | Dichloro[1,3-bis(2,6-di-4-heptylphenyl)imidazol-2- |
| PEPPSI(HeptCl) | ylidene](3-chloropyridyl)palladium(II) |
| Pd-PEPPSI-IPent | 1,3-bis[2,6-bis(1-ethylpropyl)phenyl]-2H-imidazole; 3- |
| | chloropyridine; dichloropalladium |
| PE | petroleum ether |
| PMA | para-Methoxyamphetamine |
| PPh₃, Ph₃P | Triphenylphosphine |
| PyBOP | (benzotriazol-1-yloxytripyrrolidinophosphonium |
| | hexafluorophosphate) |
| Sat. | saturated |
| SFC | Supercritical fluid chromatography |
| SiO₂ | silicon dioxide |
| SOCl₂ | thionyl chloride |
| SPDP | succinimidyl 3-(2-pyridyldithio)propionate |
| TBAF | Tetra-n-butylammonium fluoride |
| TBD | Triazabicyclodecene |
| t-BiXphos-Pd-G3 | [(2-Di-tert-butylphosphino-2′,4′,6′-triisopropyl-1,1′- |
| | biphenyl)-2-(2′-amino-1,1′-biphenyl)] palladium(II) |
| | methanesulfonate |
| t-BuNH₂ | tert-Butylamine |
| TEA | triethylamine |
| TFA | trifluoroacetic acid |
| THF | tetrahydrofuran |
| THPTA | tris-hydroxypropyltriazolylmethylamine |
| TLC | thin layer chromatography |
| TMPMgCl | 2,2,6,6 Tetramethylpiperidinylmagnesium chloride |
| tol., PhCH₃ | toluene |
| TSO₂ | thiophene sulfone |

### Example 1. General Schemes

The compounds of the present invention can by synthesized in a modular manner using techniques known to the skilled artisan. Provided in this example are general strategies for linking a USP28 Targeting Ligand described herein to a Ubiquitinated Protein Targeting Ligand described herein. These strategies can be used to install multiple linking moieties together (for example Linker-A and Linker-B) in a stepwise fashion. The reagents listed in this example are non-limiting reagents to perform routine chemical reactions and can be readily substituted for other reagents known in the art as desired.

### Example 1A. Attachment of Triazole-Containing Alkyl or Polyethylene Glycol Chains as Linker

### For Linear Alkyl:

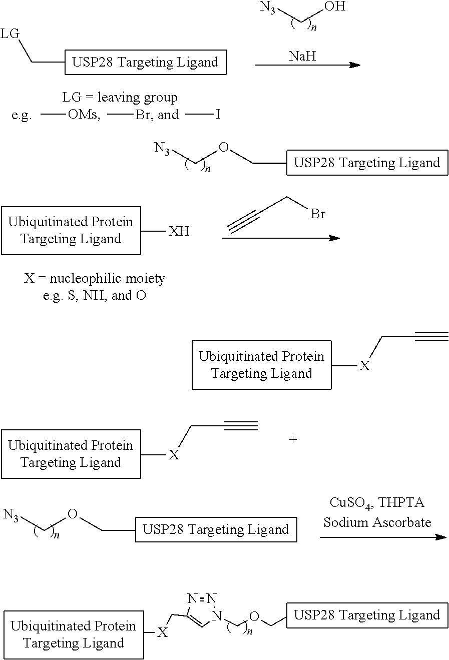

### For Polyethylene Glycol:

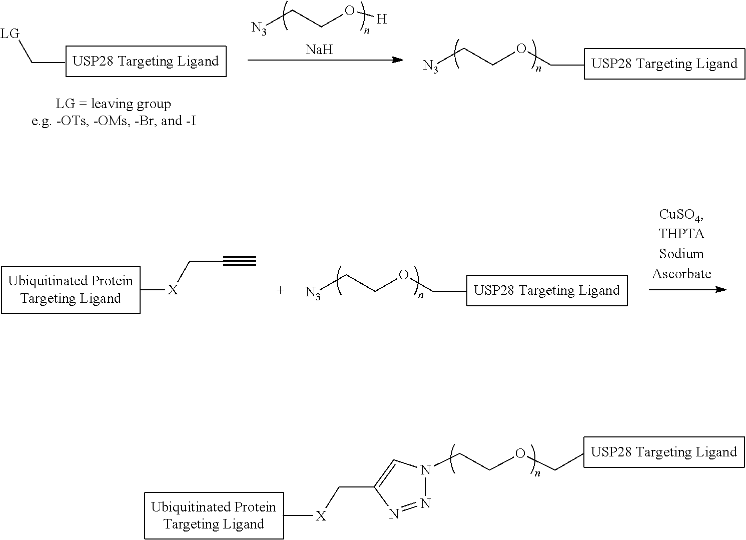

In certain embodiments, the reactive groups on the ligands shown herein are switched. For example, the nucleophilic moiety is bonded to the USP28 Targeting Ligand and the leaving group is on the Ubiquitinated Protein Targeting Ligand.

### Example 1B. Attachment of Succinimide-Containing Groups as Linker

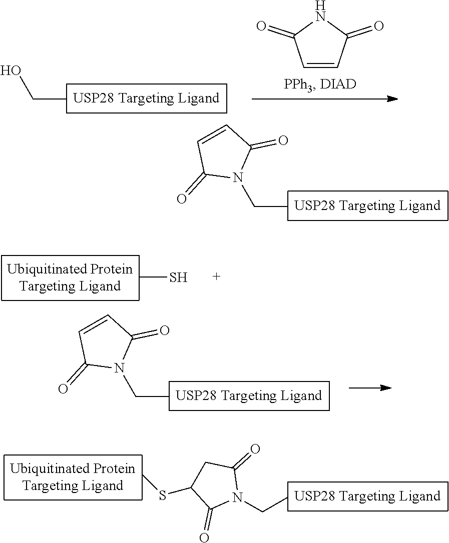

In certain embodiments, the reactive groups on the ligands shown herein are switched. For example, the electrophilic maleimide moiety is bonded to the Ubiquitinated Protein Targeting Ligand and the nucleophilic moiety is on the USP28 Targeting Ligand.

### Example 1C. Attachment of Amide-Containing Alkyl or Polyethylene Glycol Chains as Linker

### For Linear Alkyl:

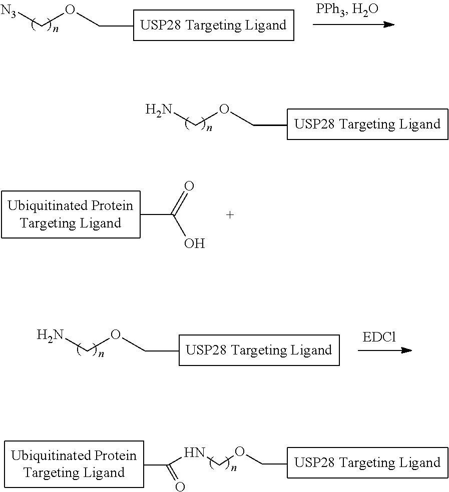

### For Polyethylene Glycol:

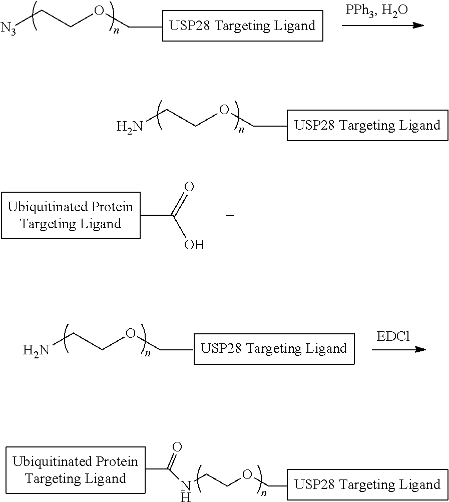

In certain embodiments, the reactive groups on the ligands shown herein are switched. For example, the amine moiety is bonded to the Ubiquitinated Protein Targeting Ligand and the carboxylic acid moiety is on the USP28 Targeting Ligand.

### Example 1D Attachment of Triazole-Containing Alkyl or Polyethylene Glycol Chains as Linker-A or Linker-B

### Linear Alkyl as Linker-a:

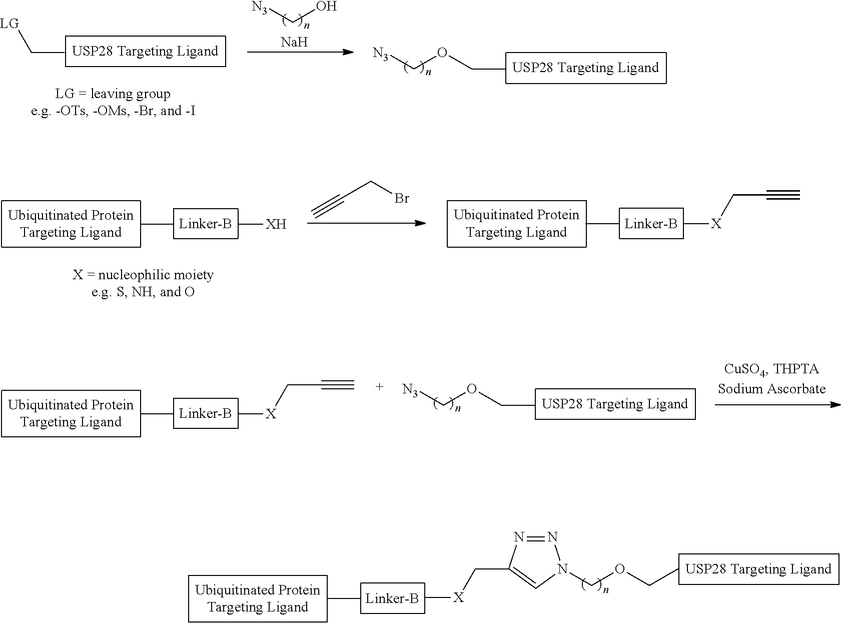

In certain embodiments, the reactive groups on the ligands shown herein are switched. For example, the leaving group moiety is bonded to the Ubiquitinated Protein Targeting Ligand and the nucleophilic moiety is on the Linker-B.

### Alternatively for Linear Alkyl as Linker-B:

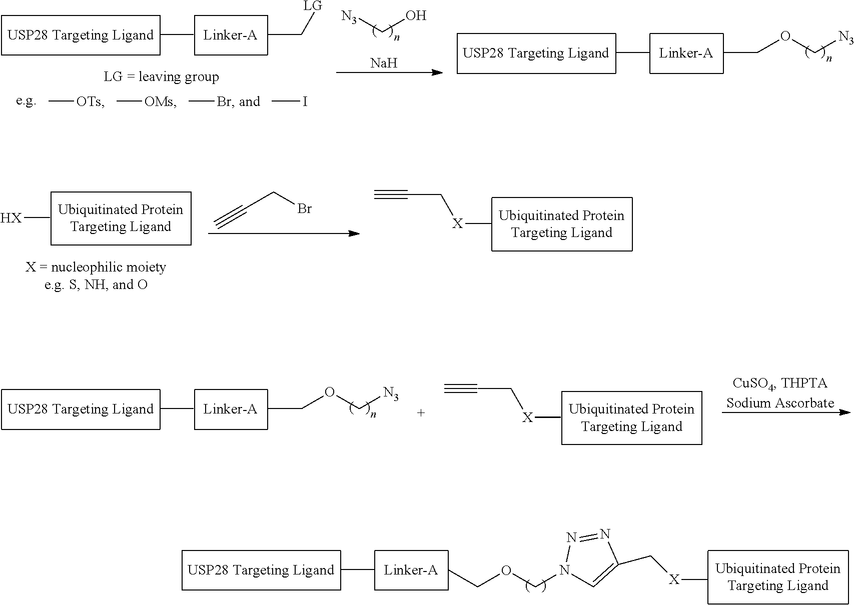

In certain embodiments, the reactive groups on the ligands shown herein are switched. For example, the leaving group moiety is bonded to the Ubiquitinated Protein Targeting Ligand and the nucleophilic moiety is on the Linker-A.

### For Polyethylene Glycol as Linker-a:

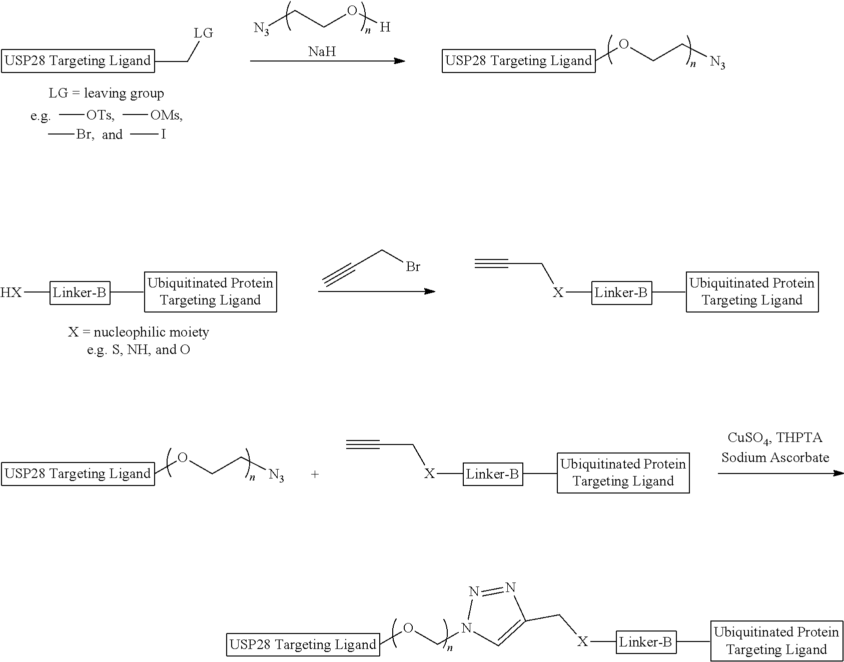

In certain embodiments, the reactive groups on the ligands shown herein are switched. For example, the leaving group moiety is bonded to the Linker-B and the nucleophilic moiety is on the USP28 Targeting Ligand.

### Alternatively, for Polyethylene Glycol as Linker-B:

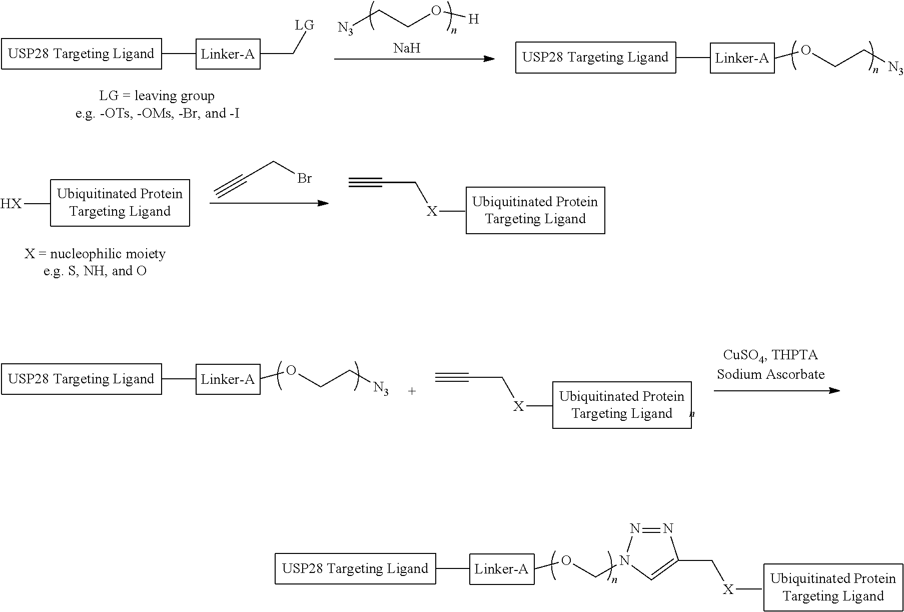

In certain embodiments, the reactive groups on the ligands shown herein are switched. For example, the leaving group moiety is bonded to the Ubiquitinated Protein Targeting Ligand and the nucleophilic moiety is on the Linker-A.

### Example 1E. Attachment of Succinimide-Containing Groups as Linker-A or Linker-B Succinimide-Containing Group as Linker-A

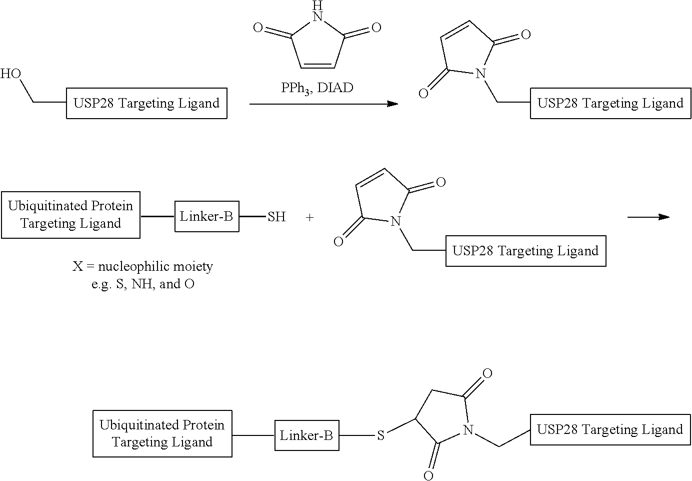

In certain embodiments, the reactive groups on the ligands shown herein are switched. For example, the electrophilic maleimide moiety is bonded to the Linker-B and the nucleophilic moiety is on the USP28 Targeting Ligand.

### Succinimide-Containing Group as Linker-B:

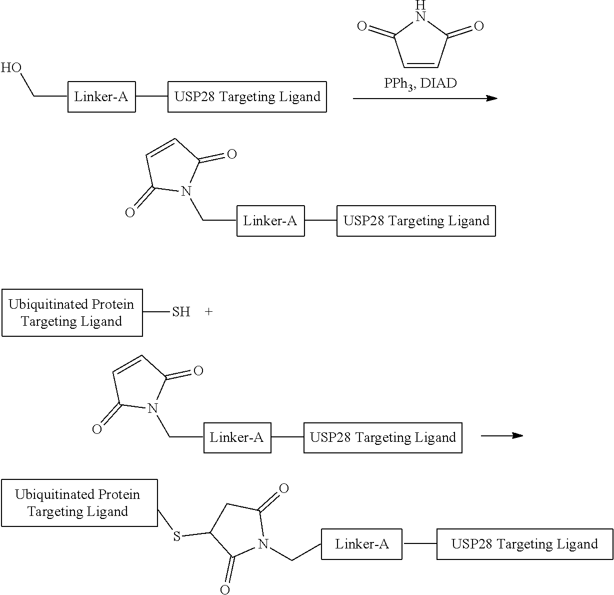

In certain embodiments, the reactive groups on the ligands shown herein are switched. For example, the electrophilic maleimide moiety is bonded to the Ubiquitinated Protein Targeting Ligand and the nucleophilic moiety is on the Linker-A.

### Example 1F. Attachment of Amide-Containing Alkyl or Polyethylene Glycol Chains as Linker

### For Linear Alkyl as Linker-A:

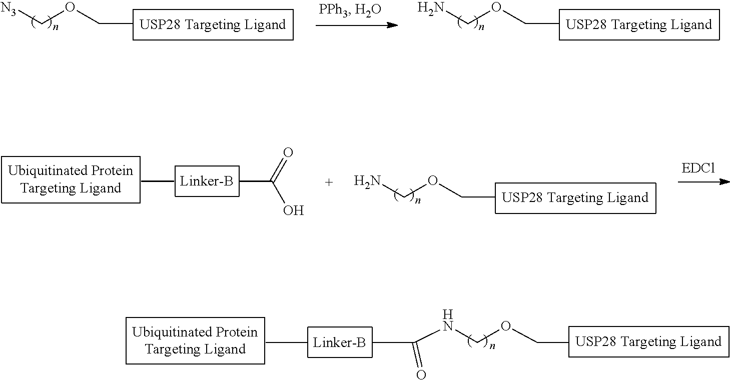

### For Polyethylene Glycol as Linker-A:

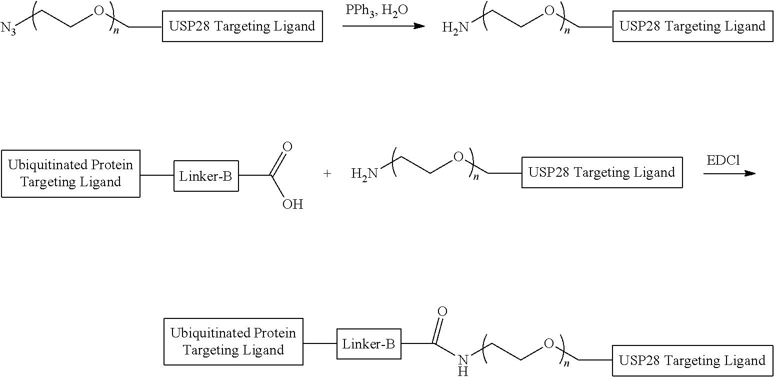

### For Linear Alkyl as Linker-B:

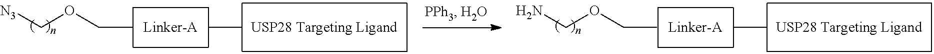

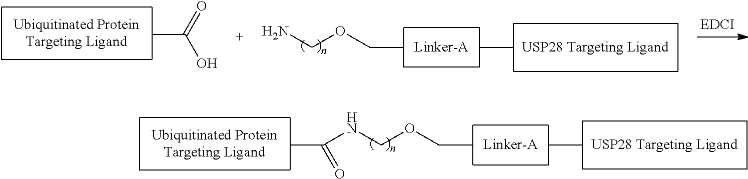

### For Polyethylene Glycol as Linker-B:

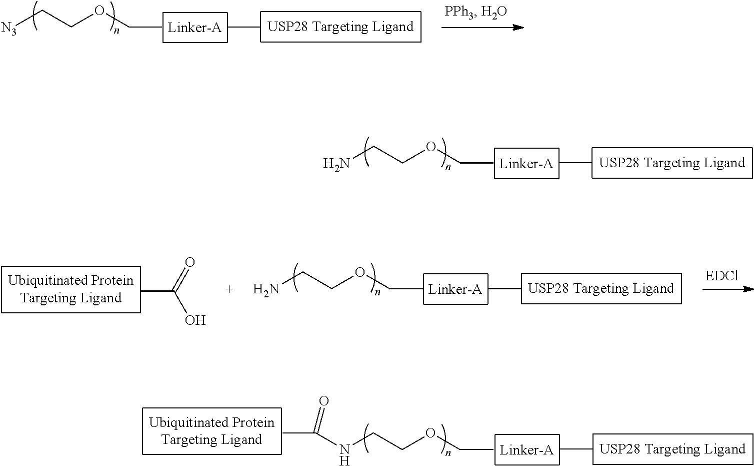

### Example 1G. Attachment Point of Linker

The compounds of the present invention can be prepared using a desired attachment point linking the Ubiquitinated Protein Targeting Ligand by preparing or procuring appropriate starting materials with corresponding functionality. For example,

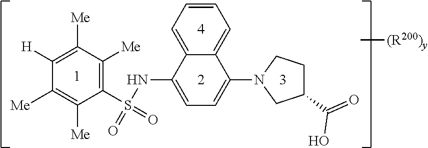

when attached to the Linker in the cycle marked with a 1 includes the following non-limiting exemplary structure:

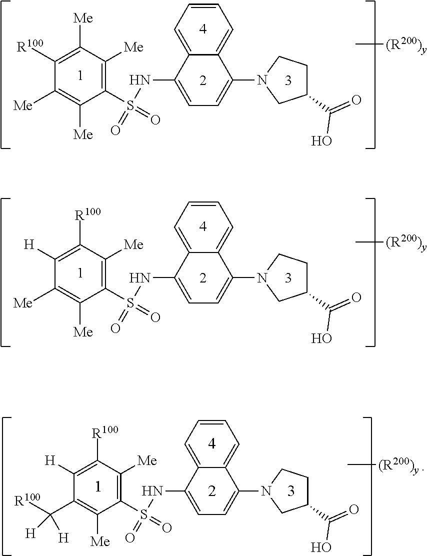

The synthesis of this KEAP1 Targeting Ligand has been reported in the literature. For example in Journal of Medicinal Chemistry (2019), 62(17), 8028-8052:

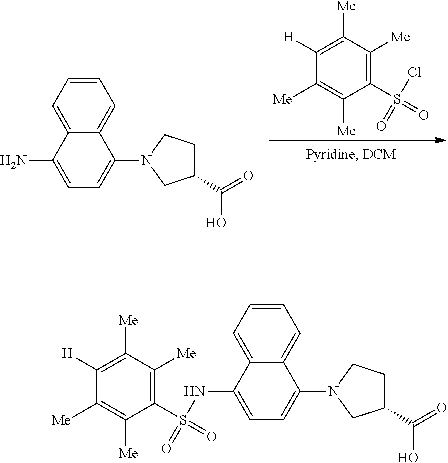 [SMILES: Nc1ccc(N2CC[C@H](C(=O)O)C2)c2ccccc12 ; Cc1cc(C)c(C)c(S(=O)(=O)Cl)c1C ; Cc1cc(C)c(C)c(S(=O)(=O)Nc2ccc(N3CC[C@H](C(=O)O)C3)c3ccccc23)c1C]

The starting materials in this synthesis can be replaced as necessary to provide functional groups that can be linked at the cycle 1 position. For example:

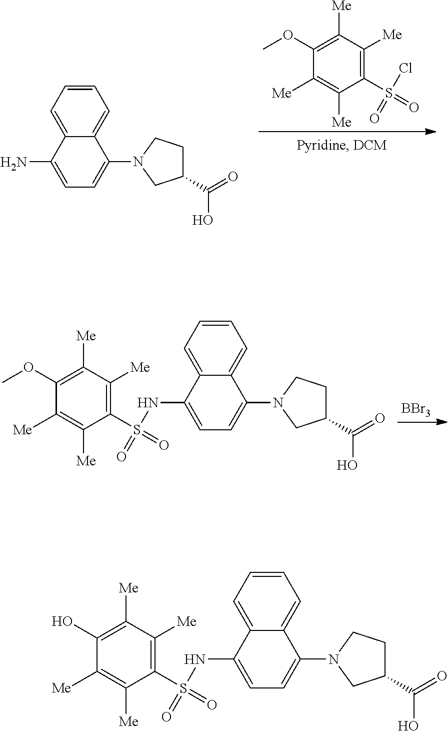

Additional transformations can be employed as needed to use other linking locations. For example:

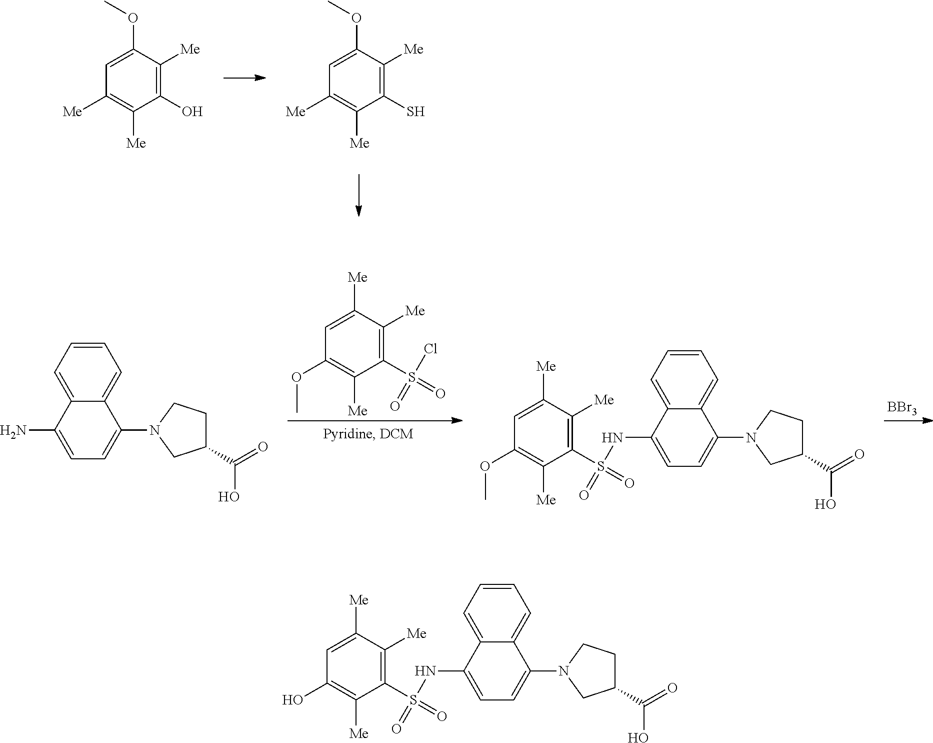

These techniques as well as other well-known reactions such as nucleophilic substitutions and coupling reactions can be used to prepare compounds that are linked differently to cycle 1 than those described above. Additional non-limiting examples of starting materials that can be employed to attach a linker to cycle 1 include:

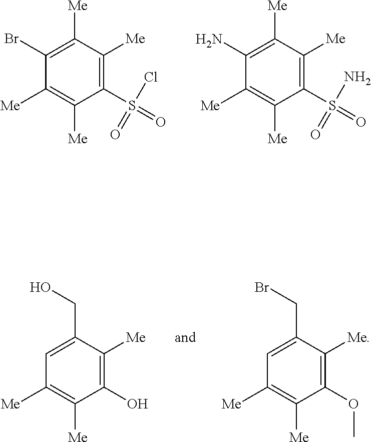

TABLE 1
Non-limiting Representative Compounds of the Present Invention

| # | MW | Structure |
|---|---|---|
| 1a | 1024.96 | 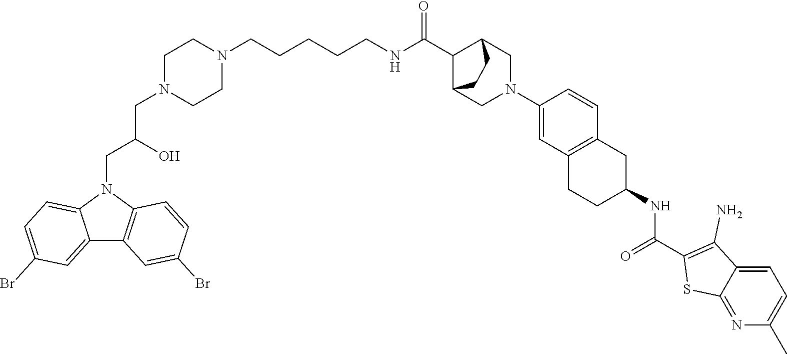 [SMILES: Cc1ccc2c(N)c(C(=O)N[C@H]3CCc4cc(N5C[C@H]6CC[C@@H](C5)C6C(=O)NCCCCCN5CCN(CC(O)Cn6c7ccc(Br)cc7c7cc(Br)ccc76)CC5)ccc4C3)sc2n1] |
| 1b | 1024.96 | 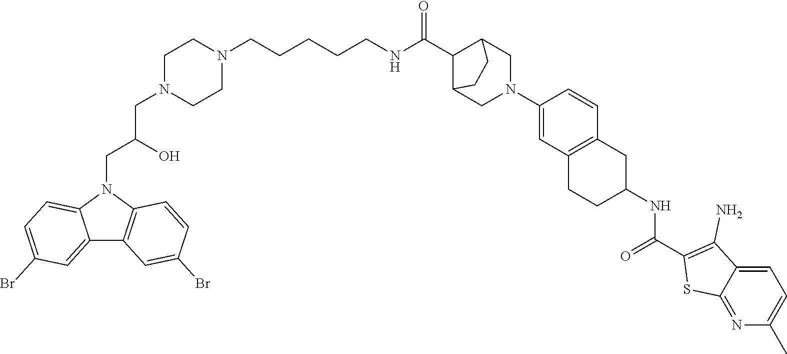 [SMILES: Cc1ccc2c(N)c(C(=O)NC3CCc4cc(N5CC6CCC(C5)C6C(=O)NCCCCCN5CCN(CC(O)Cn6c7ccc(Br)cc7c7cc(Br)ccc76)CC5)ccc4C3)sc2n1] |
| 2a | 1033.26 | 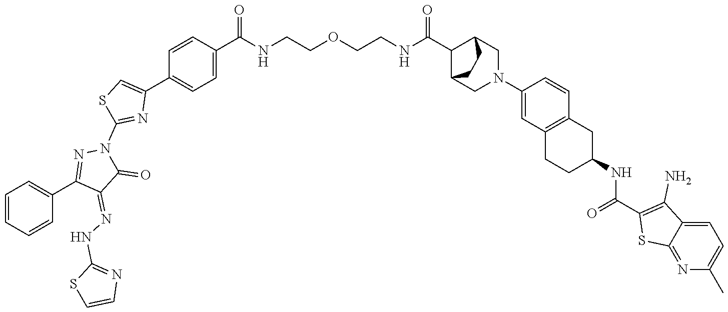 [SMILES: Cc1ccc2c(N)c(C(=O)N[C@H]3CCc4cc(N5C[C@H]6CC[C@@H](C5)C6C(=O)NCCOCCNC(=O)c5ccc(-c6csc(N7N=C(c8ccccc8)/C(=N\Nc8nccs8)C7=O)n6)cc5)ccc4C3)sc2n1] |
| 2b | 1033.26 | 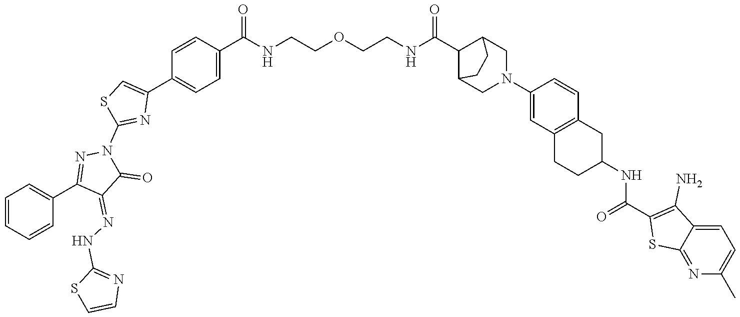 [SMILES: Cc1ccc2c(N)c(C(=O)NC3CCc4cc(N5CC6CCC(C5)C6C(=O)NCCOCCNC(=O)c5ccc(-c6csc(N7N=C(c8ccccc8)/C(=N\Nc8nccs8)C7=O)n6)cc5)ccc4C3)sc2n1] |
| 3 | 1033.26 | 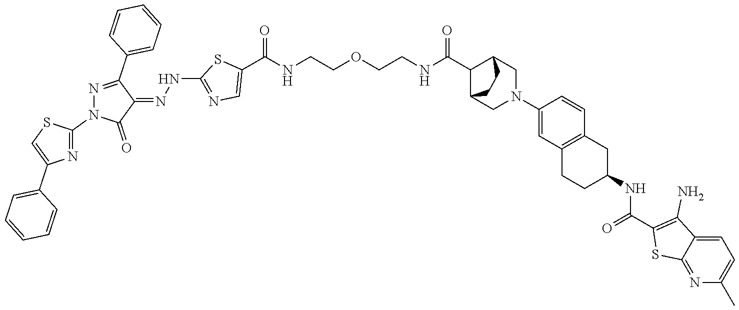 [SMILES: Cc1ccc2c(N)c(C(=O)N[C@H]3CCc4cc(N5C[C@H]6CC[C@@H](C5)C6C(=O)NCCOCCNC(=O)c5cnc(N/N=C6/C(=O)N(c7nc(-c8ccccc8)cs7)N=C6c6ccccc6)s5)ccc4C3)sc2n1] |
| 4 | 1109.04 | 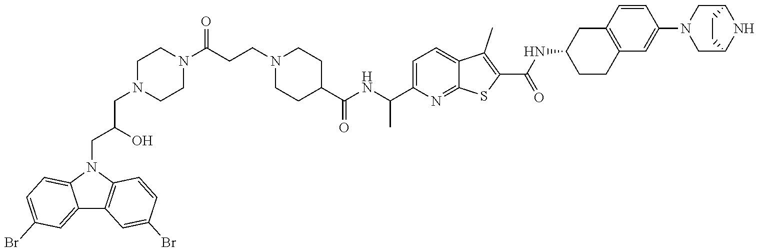 [SMILES: Cc1c(C(=O)N[C@H]2CCc3cc(N4C[C@H]5CC[C@@H](C4)N5)ccc3C2)sc2nc(C(C)NC(=O)C3CCN(CCC(=O)N4CCN(CC(O)Cn5c6ccc(Br)cc6c6cc(Br)ccc65)CC4)CC3)ccc12] |
| 5 | 1087.35 | 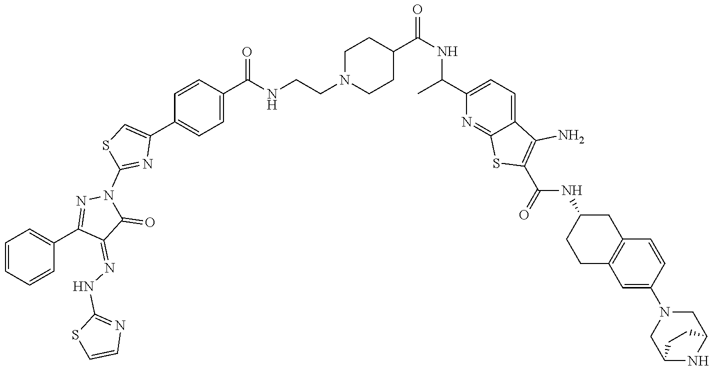 [SMILES: CC(NC(=O)C1CCN(CCNC(=O)c2ccc(-c3csc(N4N=C(c5ccccc5)/C(=N\Nc5nccs5)C4=O)n3)cc2)CC1)c1ccc2c(N)c(C(=O)N[C@H]3CCc4cc(N5C[C@H]6CC[C@@H](C5)N6)ccc4C3)sc2n1] |
| 6a | 1087.35 | 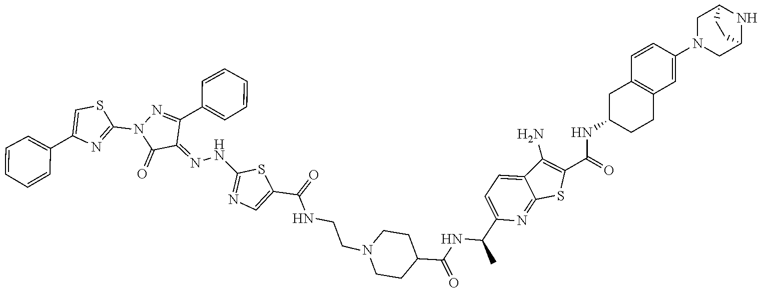 [SMILES: C[C@@H](NC(=O)C1CCN(CCNC(=O)c2cnc(N/N=C3/C(=O)N(c4nc(-c5ccccc5)cs4)N=C3c3ccccc3)s2)CC1)c1ccc2c(N)c(C(=O)N[C@H]3CCc4cc(N5C[C@H]6CC[C@@H](C5)N6)ccc4C3)sc2n1] |
| 6b | 1087.35 | 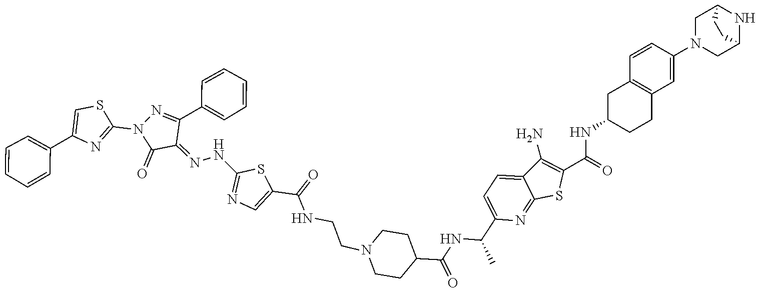 [SMILES: C[C@H](NC(=O)C1CCN(CCNC(=O)c2cnc(N/N=C3/C(=O)N(c4nc(-c5ccccc5)cs4)N=C3c3ccccc3)s2)CC1)c1ccc2c(N)c(C(=O)N[C@H]3CCc4cc(N5C[C@H]6CC[C@@H](C5)N6)ccc4C3)sc2n1] |
| 7a | 967.85 | 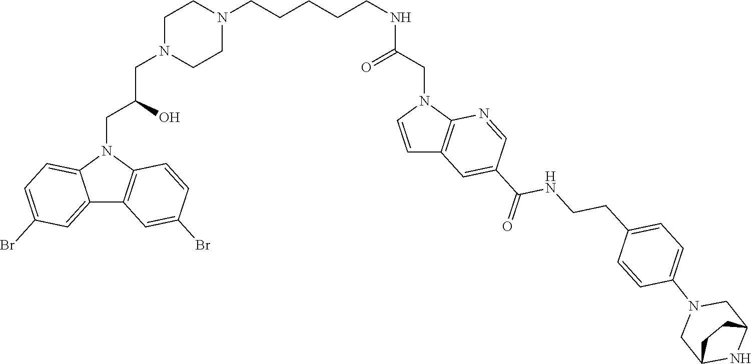 [SMILES: O=C(Cn1ccc2cc(C(=O)NCCc3ccc(N4C[C@H]5CC[C@@H](C4)N5)cc3)cnc21)NCCCCCN1CCN(C[C@@H](O)Cn2c3ccc(Br)cc3c3cc(Br)ccc32)CC1] |
| 7b | 967.85 | 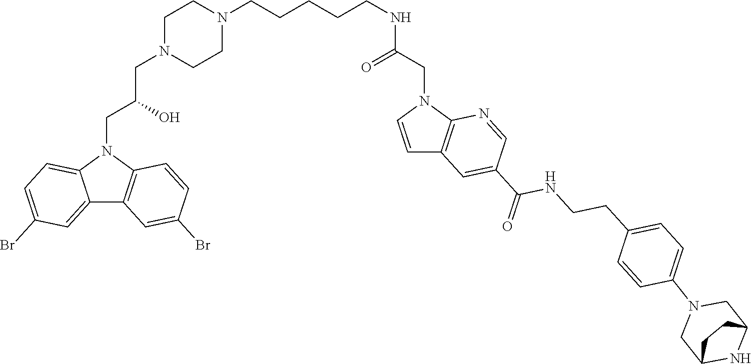 [SMILES: O=C(Cn1ccc2cc(C(=O)NCCc3ccc(N4C[C@H]5CC[C@@H](C4)N5)cc3)cnc21)NCCCCCN1CCN(C[C@H](O)Cn2c3ccc(Br)cc3c3cc(Br)ccc32)CC1] |
| 8 | 976.15 | 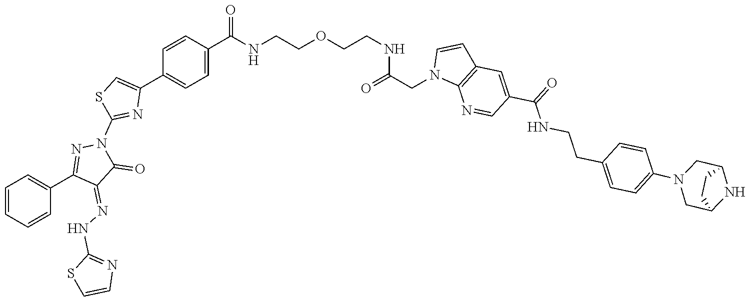 [SMILES: O=C(Cn1ccc2cc(C(=O)NCCc3ccc(N4C[C@H]5CC[C@@H](C4)N5)cc3)cnc21)NCCOCCNC(=O)c1ccc(-c2csc(N3N=C(c4ccccc4)/C(=N\Nc4nccs4)C3=O)n2)cc1] |
| 9 | 976.15 | 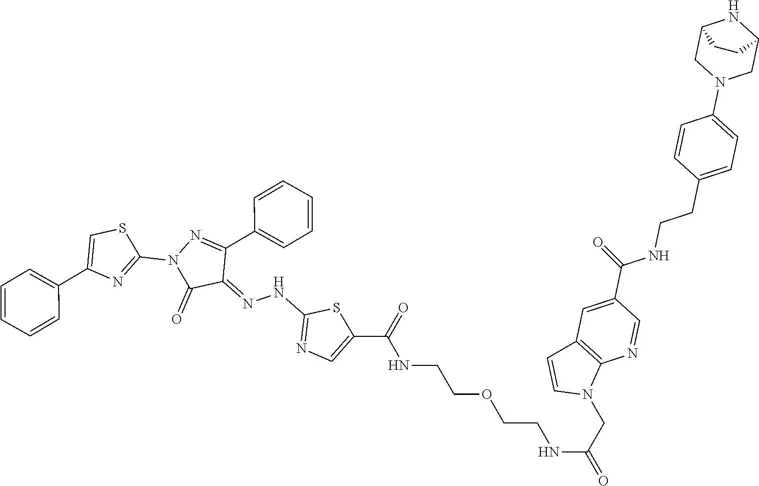 [SMILES: O=C(Cn1ccc2cc(C(=O)NCCc3ccc(N4C[C@H]5CC[C@@H](C4)N5)cc3)cnc21)NCCOCCNC(=O)c1cnc(N/N=C2/C(=O)N(c3nc(-c4ccccc4)cs3)N=C2c2ccccc2)s1] |
| 10 | 903.10 | 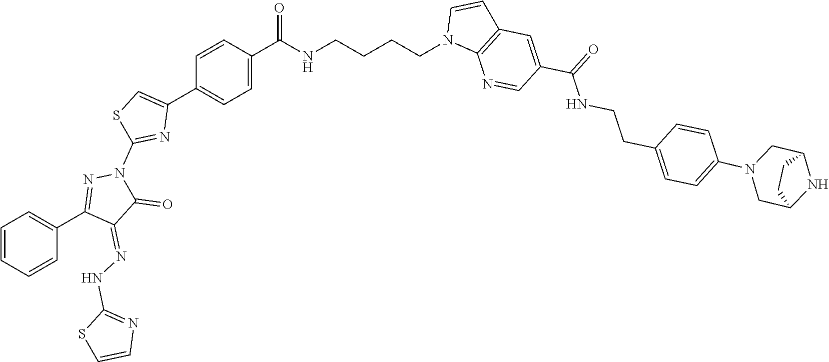 [SMILES: O=C(NCCCCn1ccc2cc(C(=O)NCCc3ccc(N4C[C@H]5CC[C@@H](C4)N5)cc3)cnc21)c1ccc(-c2csc(N3N=C(c4ccccc4)/C(=N\Nc4nccs4)C3=O)n2)cc1] |
| 11 | 903.10 | 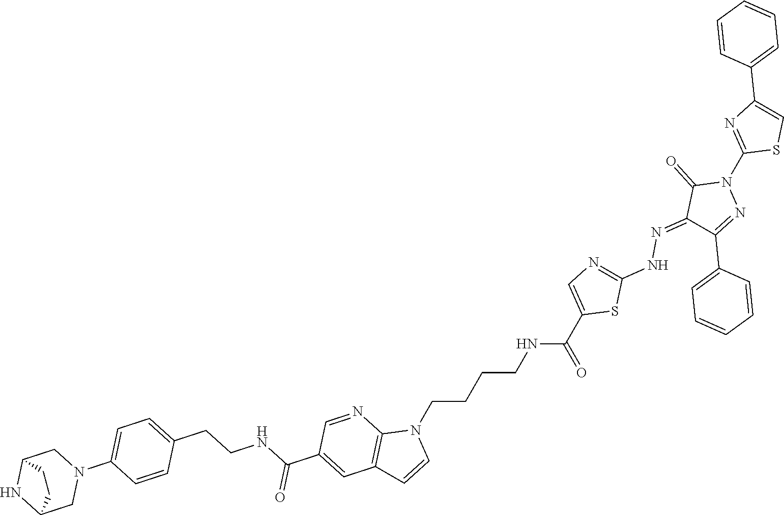 |
| 12 | 1051.99 | 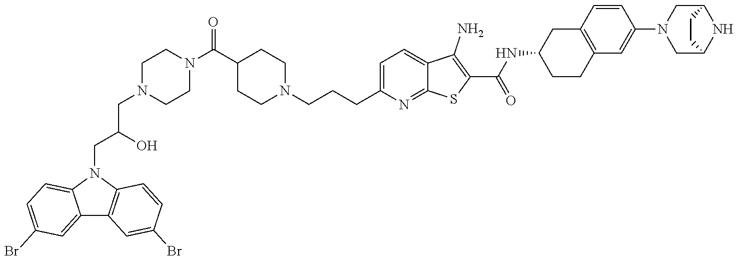 [SMILES: Nc1c(C(=O)N[C@H]2CCc3cc(N4C[C@H]5CC[C@@H](C4)N5)ccc3C2)sc2nc(CCCN3CCC(C(=O)N4CCN(CC(O)Cn5c6ccc(Br)cc6c6cc(Br)ccc65)CC4)CC3)ccc12] |
| 13 | 991.22 | 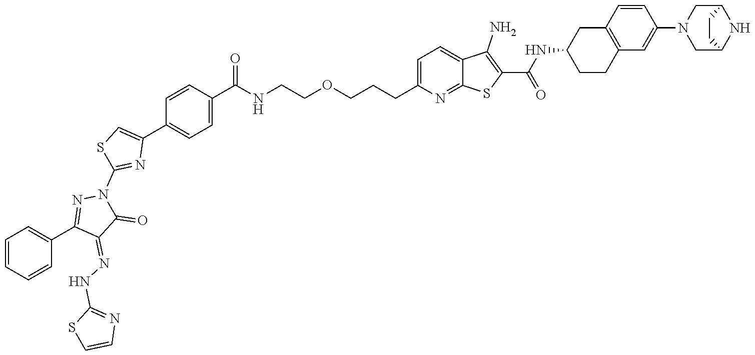 [SMILES: Nc1c(C(=O)N[C@H]2CCc3cc(N4C[C@H]5CC[C@@H](C4)N5)ccc3C2)sc2nc(CCCOCCNC(=O)c3ccc(-c4csc(N5N=C(c6ccccc6)/C(=N\Nc6nccs6)C5=O)n4)cc3)ccc12] |
| 14 | 991.22 | 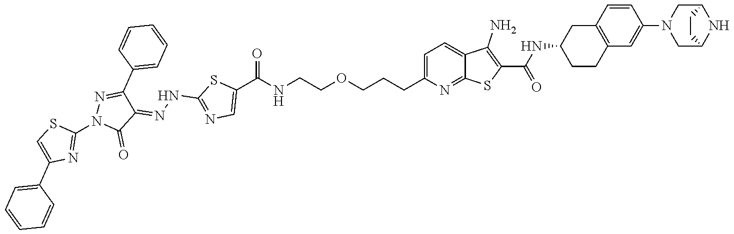 [SMILES: Nc1c(C(=O)N[C@H]2CCc3cc(N4C[C@H]5CC[C@@H](C4)N5)ccc3C2)sc2nc(CCCOCCNC(=O)c3cnc(N/N=C4/C(=O)N(c5nc(-c6ccccc6)cs5)N=C4c4ccccc4)s3)ccc12] |
| 15 | 996.91 | 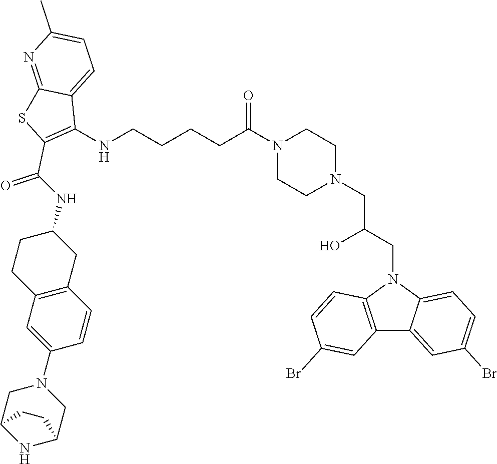 [SMILES: Cc1ccc2c(NCCCCC(=O)N3CCN(CC(O)Cn4c5ccc(Br)cc5c5cc(Br)ccc54)CC3)c(C(=O)N[C@H]3CCc4cc(N5C[C@H]6CC[C@@H](C5)N6)ccc4C3)sc2n1] |
| 16 | 975.22 | 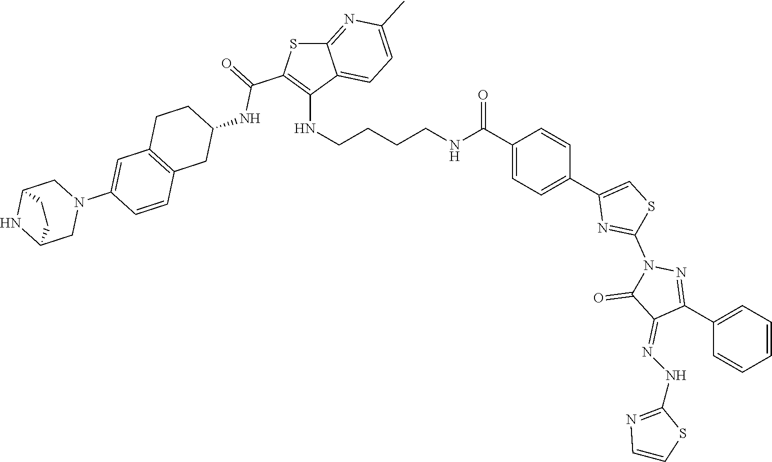 [SMILES: Cc1ccc2c(NCCCCNC(=O)c3ccc(-c4csc(N5N=C(c6ccccc6)/C(=N\Nc6nccs6)C5=O)n4)cc3)c(C(=O)N[C@H]3CCc4cc(N5C[C@H]6CC[C@@H](C5)N6)ccc4C3)sc2n1] |
| 17 | 975.22 | 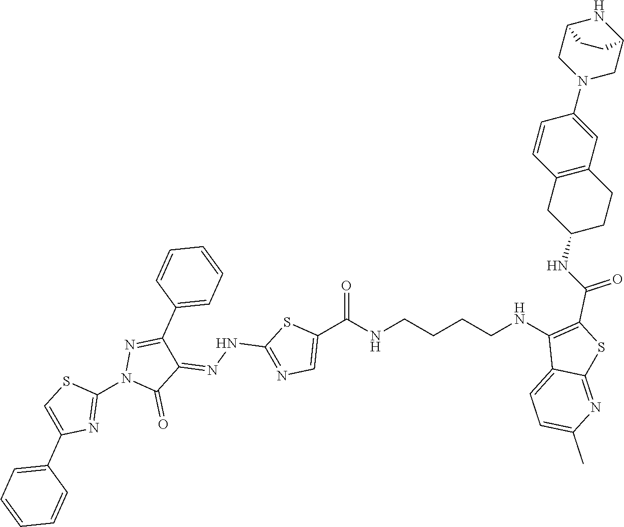 [SMILES: Cc1ccc2c(NCCCCNC(=O)c3cnc(N/N=C4/C(=O)N(c5nc(-c6ccccc6)cs5)N=C4c4ccccc4)s3)c(C(=O)N[C@H]3CCc4cc(N5C[C@H]6CC[C@@H](C5)N6)ccc4C3)sc2n1] |
| 18 | 945.82 | 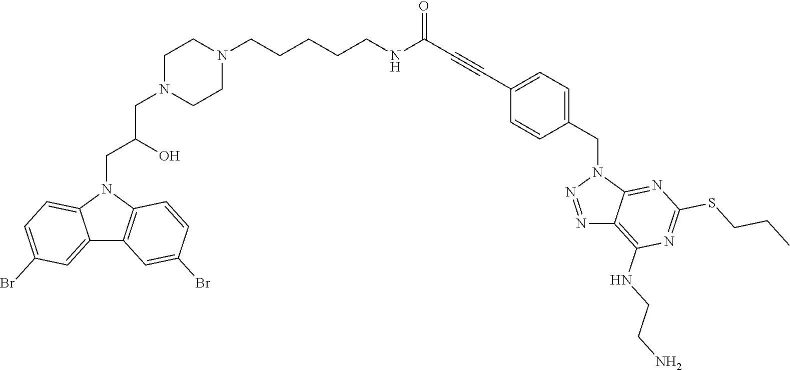 [SMILES: CCCSc1nc(NCCN)c2nnn(Cc3ccc(C#CC(=O)NCCCCCN4CCN(CC(O)Cn5c6ccc(Br)cc6c6cc(Br)ccc65)CC4)cc3)c2n1] |
| 19 | 949.86 | 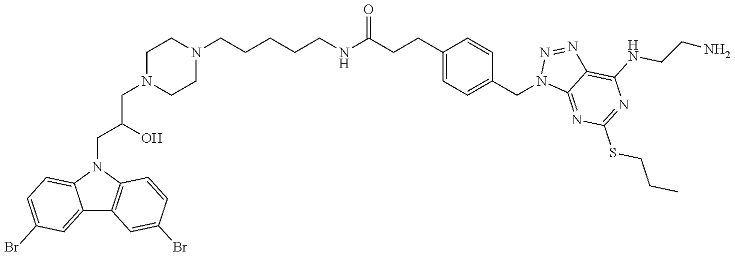 [SMILES: CCCSc1nc(NCCN)c2nnn(Cc3ccc(CCC(=O)NCCCCCN4CCN(CC(O)Cn5c6ccc(Br)cc6c6cc(Br)ccc65)CC4)cc3)c2n1] |
| 20 | 921.80 | 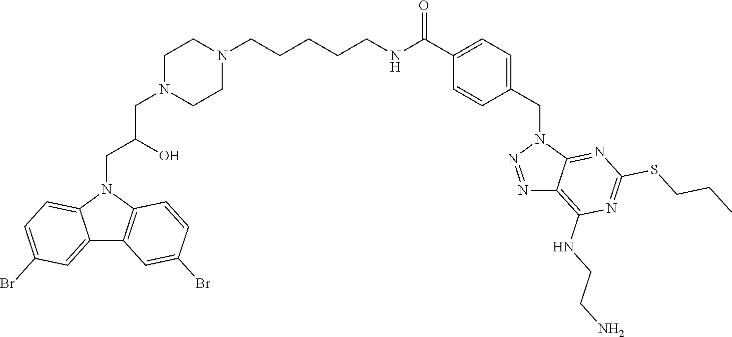 [SMILES: CCCSc1nc(NCCN)c2nnn(Cc3ccc(C(=O)NCCCCCN4CCN(CC(O)Cn5c6ccc(Br)cc6c6cc(Br)ccc65)CC4)cc3)c2n1] |
| 21 | 870.19 | 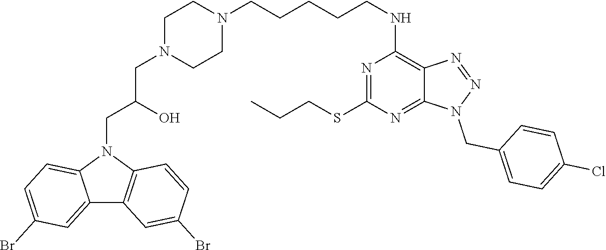 [SMILES: CCCSc1nc(NCCCCCN2CCN(CC(O)Cn3c4ccc(Br)cc4c4cc(Br)ccc43)CC2)c2nnn(Cc3ccc(Cl)cc3)c2n1] |
| 22 | 954.12 | 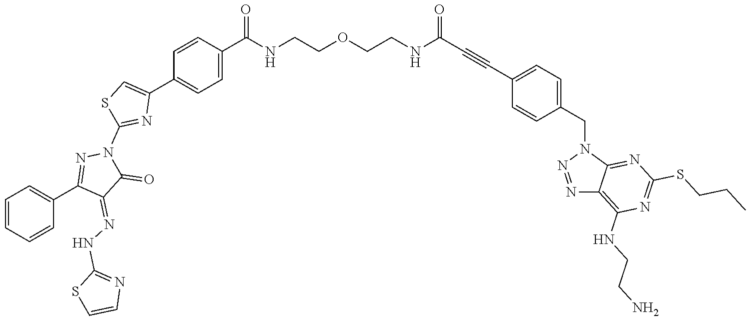 [SMILES: CCCSc1nc(NCCN)c2nnn(Cc3ccc(C#CC(=O)NCCOCCNC(=O)c4ccc(-c5csc(N6N=C(c7ccccc7)/C(=N\Nc7nccs7)C6=O)n5)cc4)cc3)c2n1] |
| 23 | 958.15 | 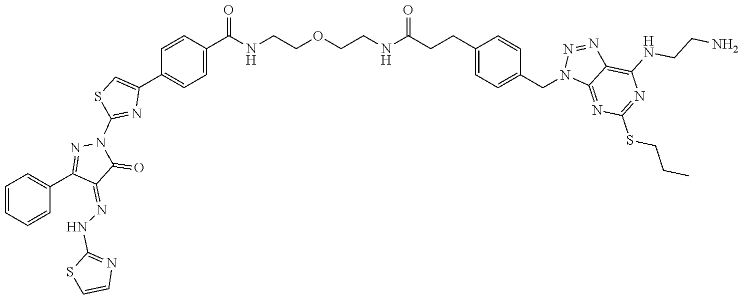 [SMILES: CCCSc1nc(NCCN)c2nnn(Cc3ccc(CCC(=O)NCCOCCNC(=O)c4ccc(-c5csc(N6N=C(c7ccccc7)/C(=N\Nc7nccs7)C6=O)n5)cc4)cc3)c2n1] |
| 24 | 930.10 | 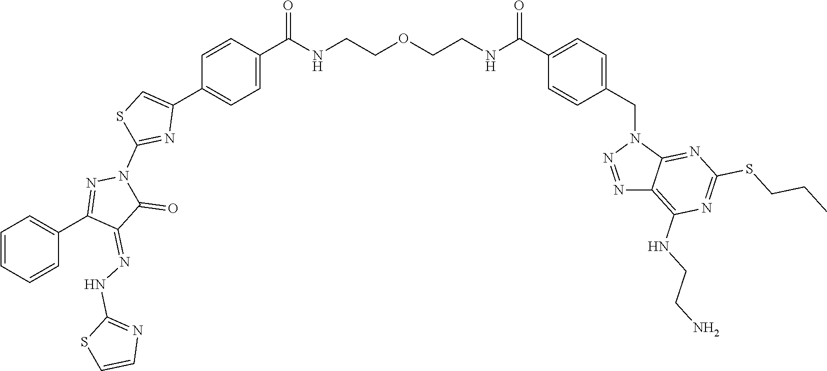 [SMILES: CCCSc1nc(NCCN)c2nnn(Cc3ccc(C(=O)NCCOCCNC(=O)c4ccc(-c5csc(N6N=C(c7ccccc7)/C(=N\Nc7nccs7)C6=O)n5)cc4)cc3)c2n1] |
| 25 | 878.45 | 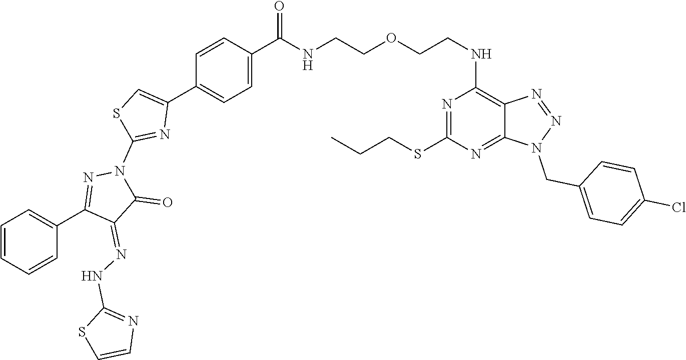 [SMILES: CCCSc1nc(NCCOCCNC(=O)c2ccc(-c3csc(N4N=C(c5ccccc5)/C(=N\Nc5nccs5)C4=O)n3)cc2)c2nnn(Cc3ccc(Cl)cc3)c2n1] |
| 26 | 954.12 | 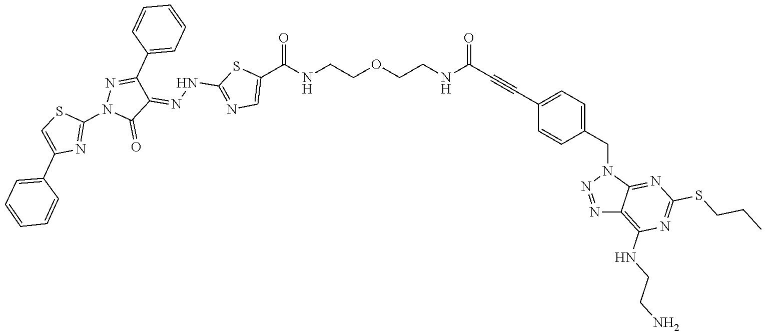 [SMILES: CCCSc1nc(NCCN)c2nnn(Cc3ccc(C#CC(=O)NCCOCCNC(=O)c4cnc(N/N=C5/C(=O)N(c6nc(-c7ccccc7)cs6)N=C5c5ccccc5)s4)cc3)c2n1] |
| 27 | 958.15 | 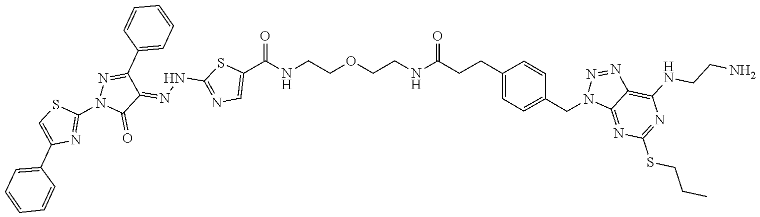 [SMILES: CCCSc1nc(NCCN)c2nnn(Cc3ccc(CCC(=O)NCCOCCNC(=O)c4cnc(N/N=C5/C(=O)N(c6nc(-c7ccccc7)cs6)N=C5c5ccccc5)s4)cc3)c2n1] |
| 28 | 930.10 | 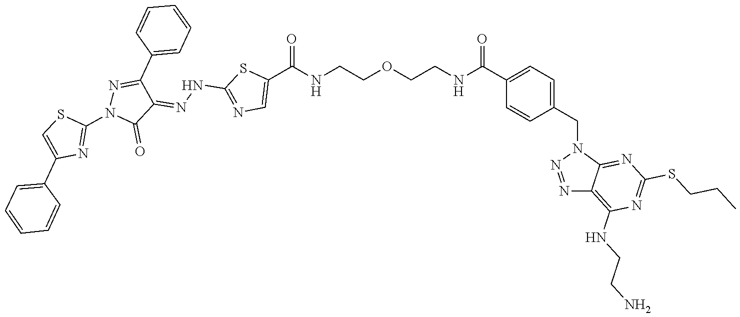 [SMILES: CCCSc1nc(NCCN)c2nnn(Cc3ccc(C(=O)NCCOCCNC(=O)c4cnc(N/N=C5/C(=O)N(c6nc(-c7ccccc7)cs6)N=C5c5ccccc5)s4)cc3)c2n1] |
| 29 | 878.45 | 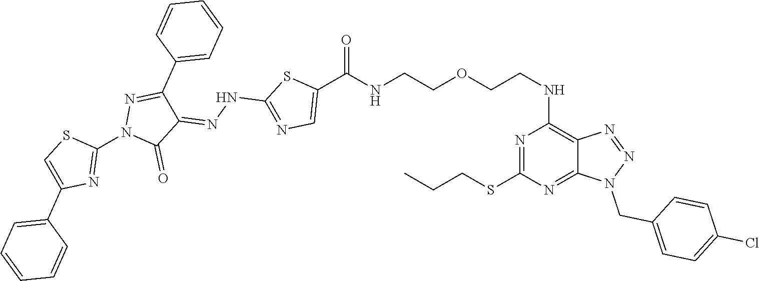 [SMILES: CCCSc1nc(NCCOCCNC(=O)c2cnc(N/N=C3/C(=O)N(c4nc(-c5ccccc5)cs4)N=C3c3ccccc3)s2)c2nnn(Cc3ccc(Cl)cc3)c2n1] |
| 30 | 1028.91 | 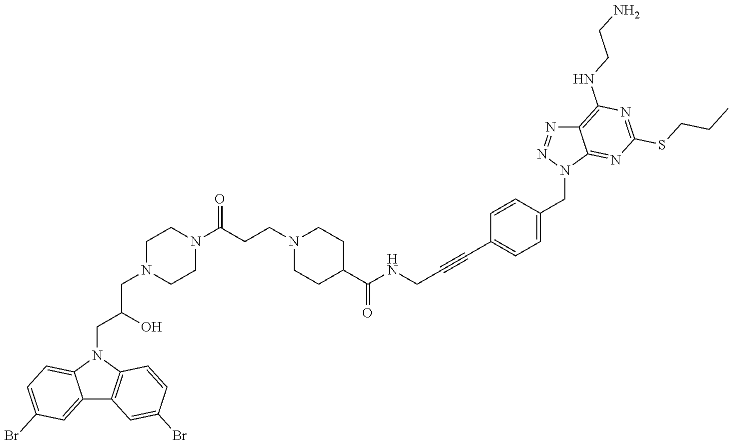 [SMILES: CCCSc1nc(NCCN)c2nnn(Cc3ccc(C#CCNC(=O)C4CCN(CCC(=O)N5CCN(CC(O)Cn6c7ccc(Br)cc7c7cc(Br)ccc76)CC5)CC4)cc3)c2n1] |
| 31 | 1032.95 | 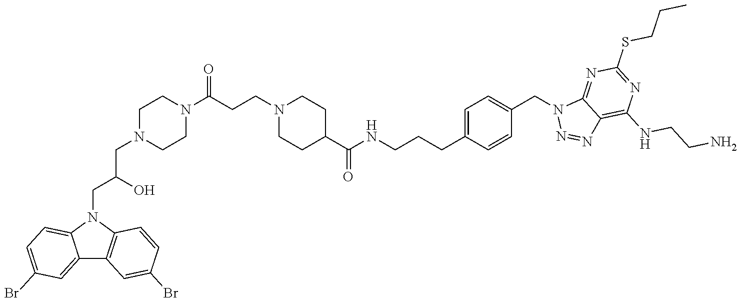 [SMILES: CCCSc1nc(NCCN)c2nnn(Cc3ccc(CCCNC(=O)C4CCN(CCC(=O)N5CCN(CC(O)Cn6c7ccc(Br)cc7c7cc(Br)ccc76)CC5)CC4)cc3)c2n1] |
| 32 | 1007.23 | 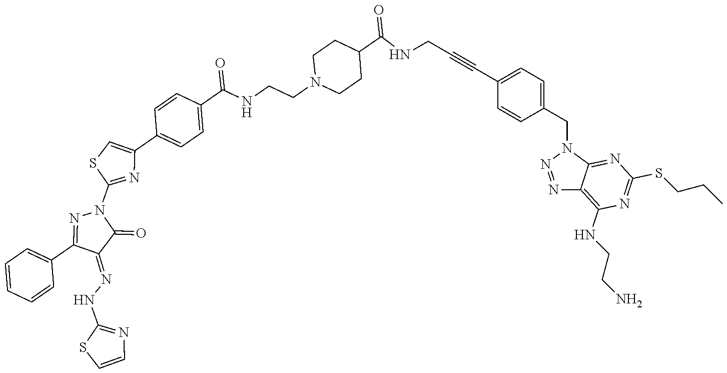 [SMILES: CCCSc1nc(NCCN)c2nnn(Cc3ccc(C#CCNC(=O)C4CCN(CCNC(=O)c5ccc(-c6csc(N7N=C(c8ccccc8)/C(=N\Nc8nccs8)C7=O)n6)cc5)CC4)cc3)c2n1] |
| 33 | 1011.27 | 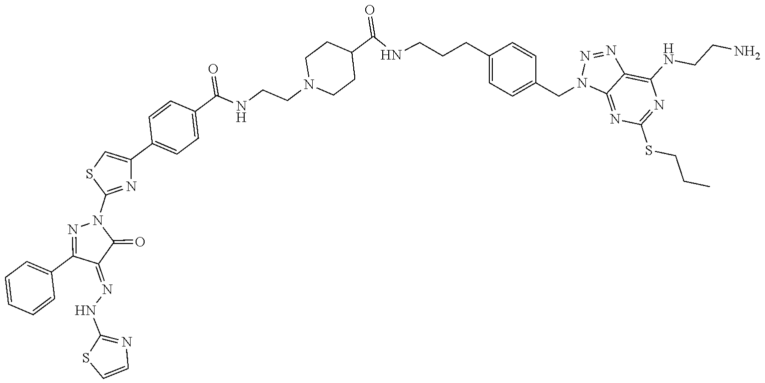 [SMILES: CCCSc1nc(NCCN)c2nnn(Cc3ccc(CCCNC(=O)C4CCN(CCNC(=O)c5ccc(-c6csc(N7N=C(c8ccccc8)/C(=N\Nc8nccs8)C7=O)n6)cc5)CC4)cc3)c2n1] |
| 34 | 1007.23 | 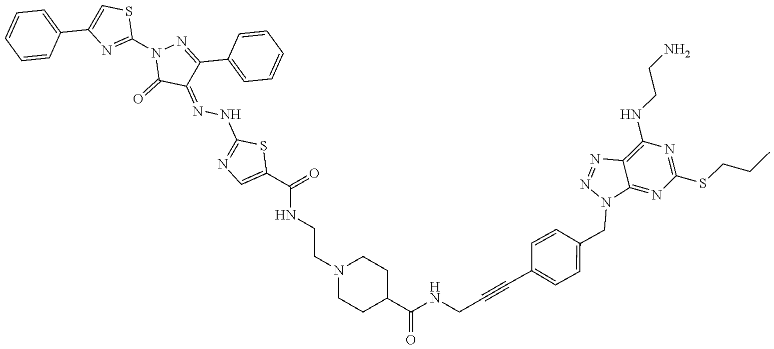 [SMILES: CCCSc1nc(NCCN)c2nnn(Cc3ccc(C#CCNC(=O)C4CCN(CCNC(=O)c5cnc(N/N=C6/C(=O)N(c7nc(-c8ccccc8)cs7)N=C6c6ccccc6)s5)CC4)cc3)c2n1] |
| 35 | 1011.27 | 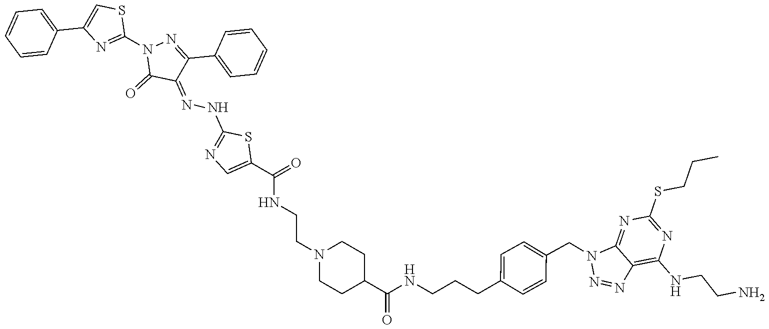 [SMILES: CCCSc1nc(NCCN)c2nnn(Cc3ccc(CCCNC(=O)C4CCN(CCNC(=O)c5cnc(N/N=C6/C(=O)N(c7nc(-c8ccccc8)cs7)N=C6c6ccccc6)s5)CC4)cc3)c2n1] |
| 36 | 945.83 | 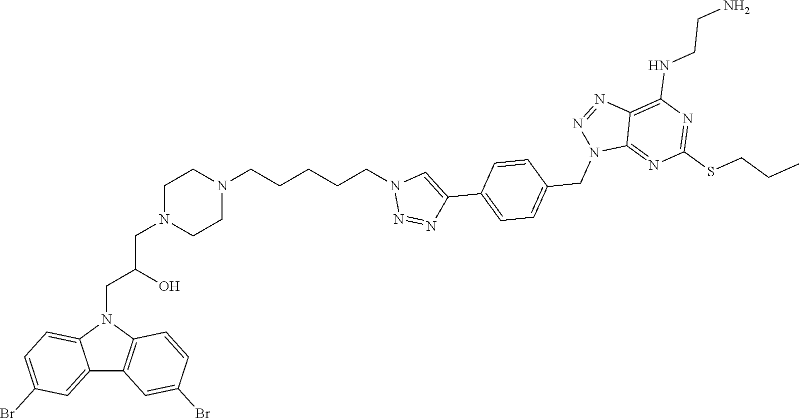 [SMILES: CCCSc1nc(NCCN)c2nnn(Cc3ccc(-c4cn(CCCCCN5CCN(CC(O)Cn6c7ccc(Br)cc7c7cc(Br)ccc76)CC5)nn4)cc3)c2n1] |
| 37 | 952.22 | 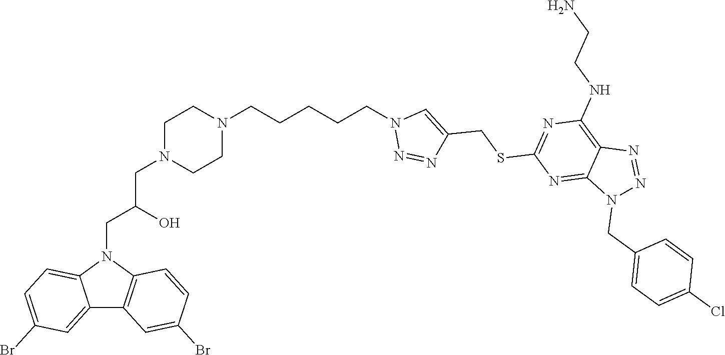 [SMILES: NCCNc1nc(SCc2cn(CCCCCN3CCN(CC(O)Cn4c5ccc(Br)cc5c5cc(Br)ccc54)CC3)nn2)nc2c1nnn2Cc1ccc(Cl)cc1] |
| 38 | 954.12 | 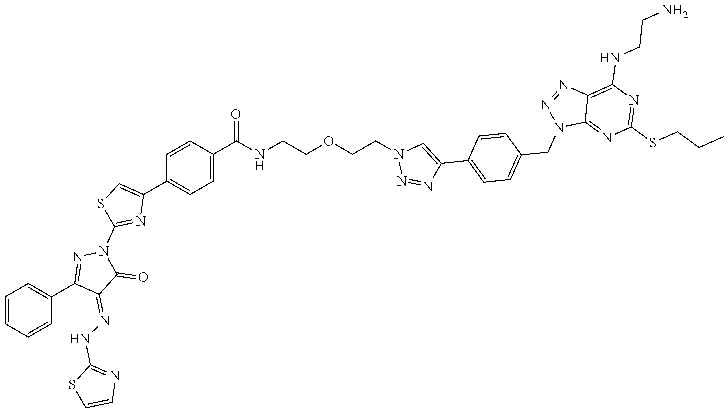 |
| 39 | 960.51 | 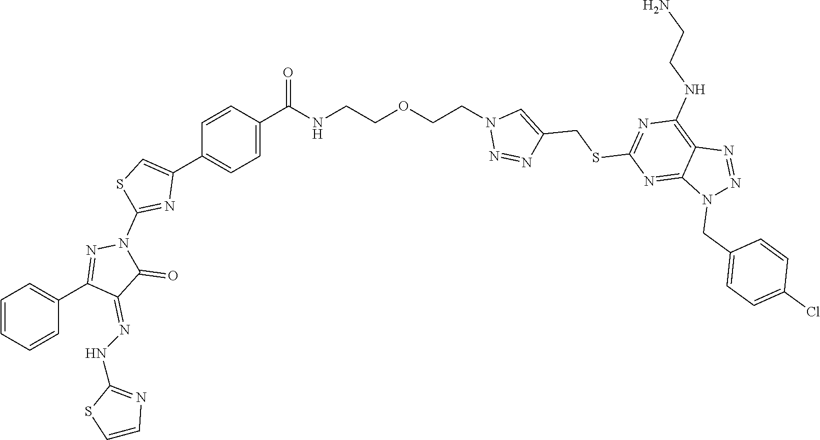 [SMILES: NCCNc1nc(SCc2cn(CCOCCNC(=O)c3ccc(-c4csc(N5N=C(c6ccccc6)/C(=N\Nc6nccs6)C5=O)n4)cc3)nn2)nc2c1nnn2Cc1ccc(Cl)cc1] |
| 40 | 954.12 | 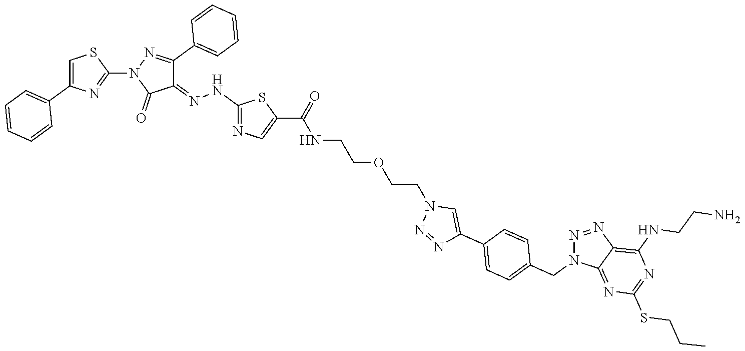 [SMILES: CCCSc1nc(NCCN)c2nnn(Cc3ccc(-c4cn(CCOCCNC(=O)c5cnc(N/N=C6/C(=O)N(c7nc(-c8ccccc8)cs7)N=C6c6ccccc6)s5)nn4)cc3)c2n1] |
| 41 | 960.51 | 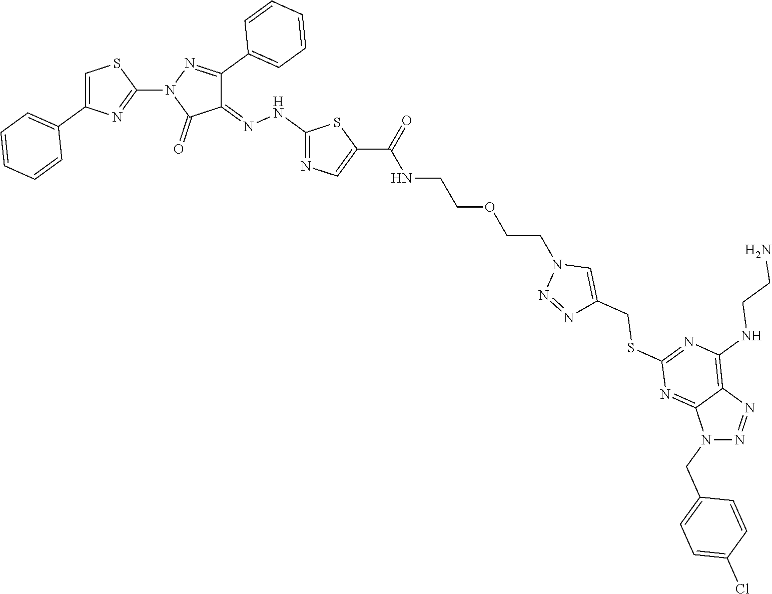 [SMILES: NCCNc1nc(SCc2cn(CCOCCNC(=O)c3cnc(N/N=C4/C(=O)N(c5nc(-c6ccccc6)cs5)N=C4c4ccccc4)s3)nn2)nc2c1nnn2Cc1ccc(Cl)cc1] |
| 42 | 953.99 | 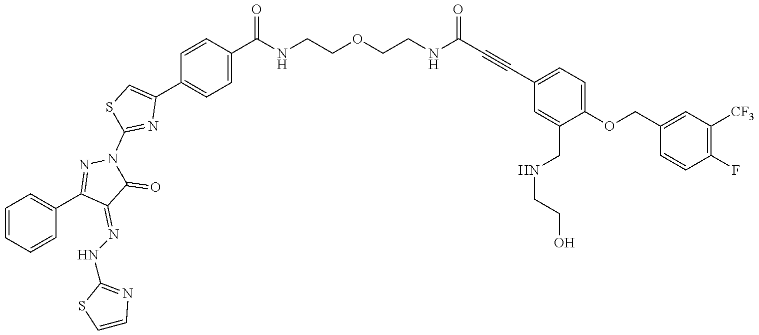 [SMILES: O=C(C#Cc1ccc(OCc2ccc(F)c(C(F)(F)F)c2)c(CNCCO)c1)NCCOCCNC(=O)c1ccc(-c2csc(N3N=C(c4ccccc4)/C(=N\Nc4nccs4)C3=O)n2)cc1] |
| 43 | 958.02 | 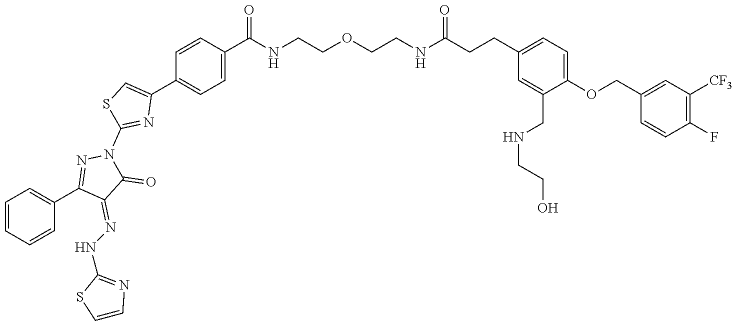 [SMILES: O=C(CCc1ccc(OCc2ccc(F)c(C(F)(F)F)c2)c(CNCCO)c1)NCCOCCNC(=O)c1ccc(-c2csc(N3N=C(c4ccccc4)/C(=N\Nc4nccs4)C3=O)n2)cc1] |
| 44 | 921.78 | 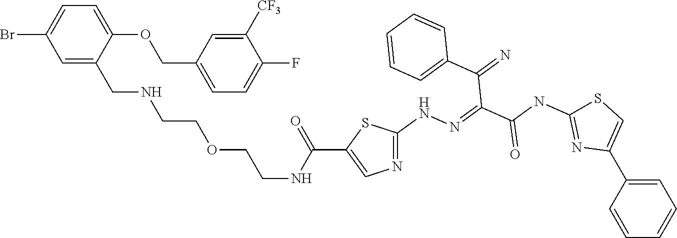 |
| 45 | 866.91 | 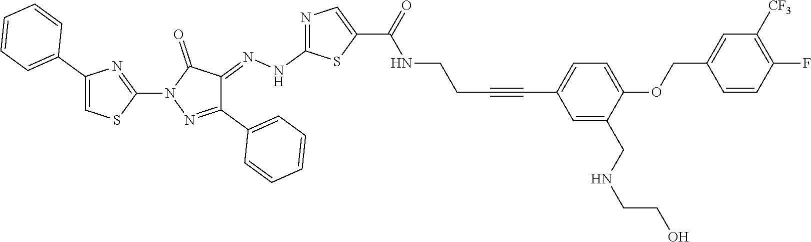 [SMILES: O=C(NCCC#Cc1ccc(OCc2ccc(F)c(C(F)(F)F)c2)c(CNCCO)c1)c1cnc(N/N=C2/C(=O)N(c3nc(-c4ccccc4)cs3)N=C2c2ccccc2)s1] |
| 46 | 870.94 | 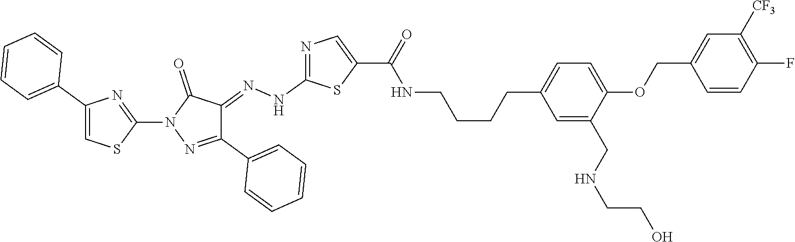 [SMILES: O=C(NCCCCc1ccc(OCc2ccc(F)c(C(F)(F)F)c2)c(CNCCO)c1)c1cnc(N/N=C2/C(=O)N(c3nc(-c4ccccc4)cs3)N=C2c2ccccc2)s1] |
| 47 | 866.91 | 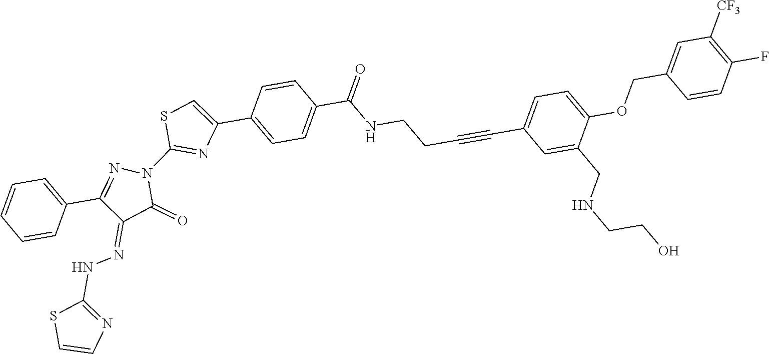 [SMILES: O=C(NCCC#Cc1ccc(OCc2ccc(F)c(C(F)(F)F)c2)c(CNCCO)c1)c1ccc(-c2csc(N3N=C(c4ccccc4)/C(=N\Nc4nccs4)C3=O)n2)cc1] |
| 48 | 870.94 | 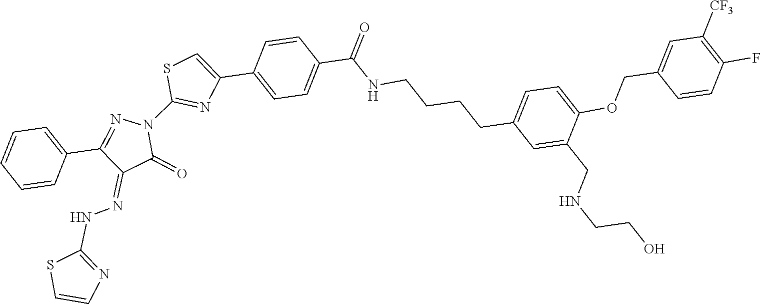 [SMILES: O=C(NCCCCc1ccc(OCc2ccc(F)c(C(F)(F)F)c2)c(CNCCO)c1)c1ccc(-c2csc(N3N=C(c4ccccc4)/C(=N\Nc4nccs4)C3=O)n2)cc1] |
| 49 | 860.59 | 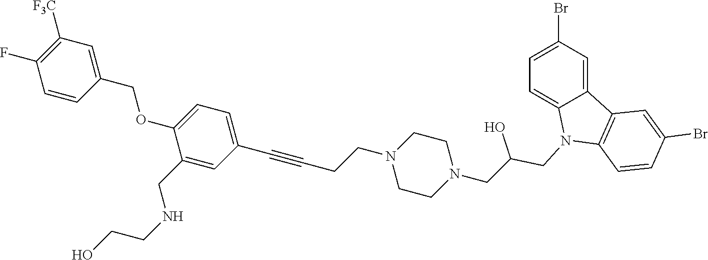 [SMILES: OCCNCc1cc(C#CCCN2CCN(CC(O)Cn3c4ccc(Br)cc4c4cc(Br)ccc43)CC2)ccc1OCc1ccc(F)c(C(F)(F)F)c1] |
| 50 | 864.62 | 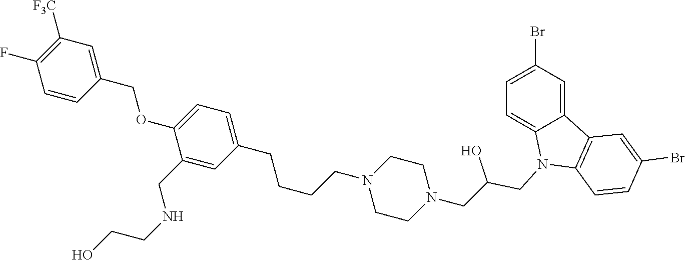 [SMILES: OCCNCc1cc(CCCCN2CCN(CC(O)Cn3c4ccc(Br)cc4c4cc(Br)ccc43)CC2)ccc1OCc1ccc(F)c(C(F)(F)F)c1] |
| 51 | 1224.05 | 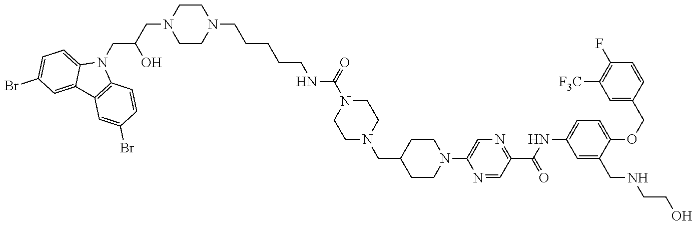 [SMILES: O=C(Nc1ccc(OCc2ccc(F)c(C(F)(F)F)c2)c(CNCCO)c1)c1cnc(N2CCC(CN3CCN(C(=O)NCCCCCN4CCN(CC(O)Cn5c6ccc(Br)cc6c6cc(Br)ccc65)CC4)CC3)CC2)cn1] |
| 52 | 947.23 | 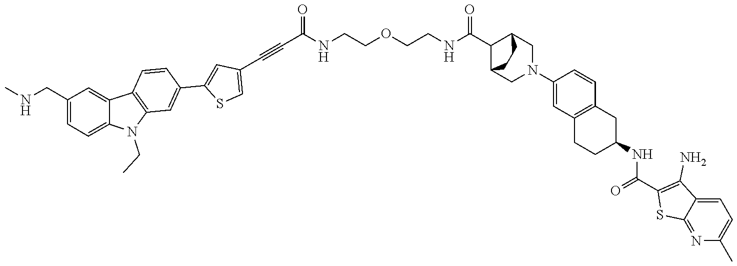 |
| 53 | 880.18 | 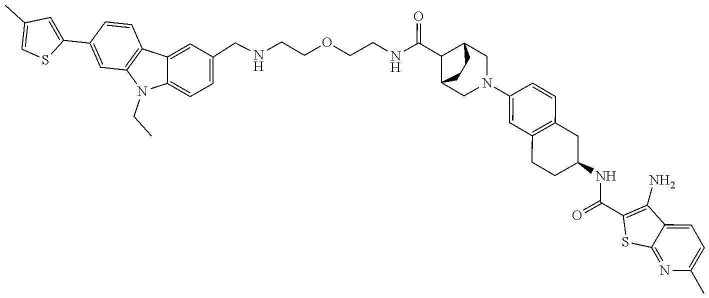 [SMILES: CCn1c2ccc(CNCCOCCNC(=O)C3[C@@H]4CC[C@H]3CN(c3ccc5c(c3)CC[C@H](NC(=O)c3sc6nc(C)ccc6c3N)C5)C4)cc2c2ccc(-c3cc(C)cs3)cc21] |
| 54 | 1062.33 | 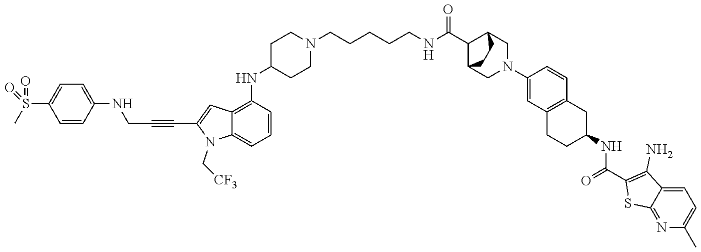 [SMILES: Cc1ccc2c(N)c(C(=O)N[C@H]3CCc4cc(N5C[C@H]6CC[C@@H](C5)C6C(=O)NCCCCCN5CCC(Nc6cccc7c6cc(C#CCNc6ccc(S(C)(=O)=O)cc6)n7CC(F)(F)F)CC5)ccc4C3)sc2n1] |
| 55 | 1029.44 | 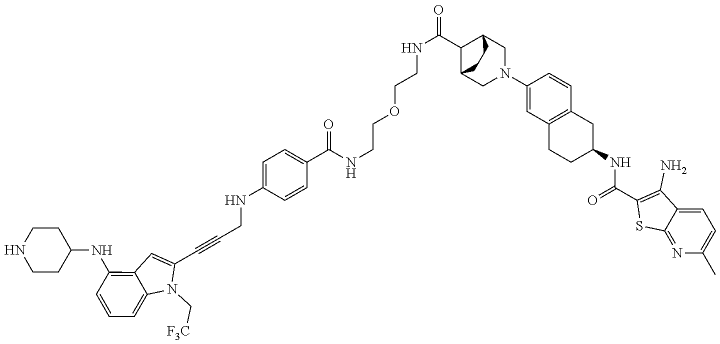 [SMILES: Cc1ccc2c(N)c(C(=O)N[C@H]3CCc4cc(N5C[C@H]6CC[C@@H](C5)C6C(=O)NCCOCCNC(=O)c5ccc(NCC#Cc6cc7c(NC8CCNCC8)cccc7n6CC(F)(F)F)cc5)ccc4C3)sc2n1] |
| 56 | 1001.33 | 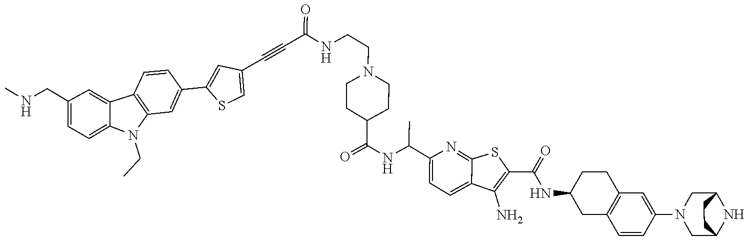 [SMILES: CCn1c2ccc(CNC)cc2c2ccc(-c3cc(C#CC(=O)NCCN4CCC(C(=O)NC(C)c5ccc6c(N)c(C(=O)N[C@H]7CCc8cc(N9C[C@H]%10CC[C@@H](C9)N%10)ccc8C7)sc6n5)CC4)cs3)cc21] |
| 57 | 934.28 | 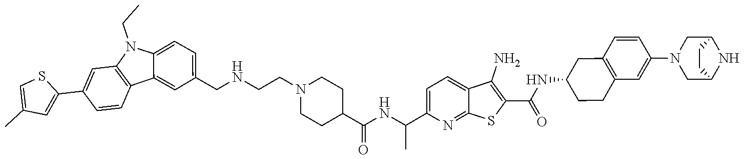 [SMILES: CCn1c2ccc(CNCCN3CCC(C(=O)NC(C)c4ccc5c(N)c(C(=O)N[C@H]6CCc7cc(N8C[C@H]9CC[C@@H](C8)N9)ccc7C6)sc5n4)CC3)cc2c2ccc(-c3cc(C)cs3)cc21] |
| 58 | 1118.40 | 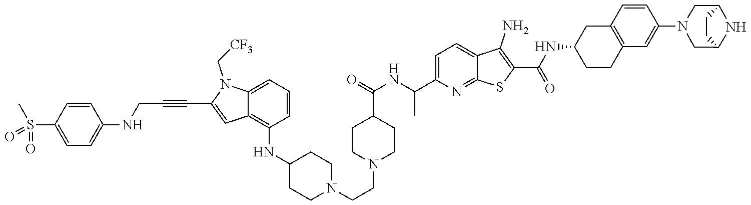 [SMILES: CC(NC(=O)C1CCN(CCN2CCC(Nc3cccc4c3cc(C#CCNc3ccc(S(C)(=O)=O)cc3)n4CC(F)(F)F)CC2)CC1)c1ccc2c(N)c(C(=O)N[C@H]3CCc4cc(N5C[C@H]6CC[C@@H](C5)N6)ccc4C3)sc2n1] |
| 59 | 1083.34 | 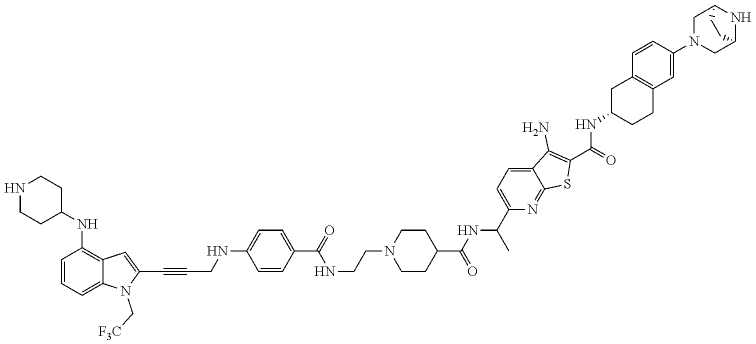 [SMILES: CC(NC(=O)C1CCN(CCNC(=O)c2ccc(NCC#Cc3cc4c(NC5CCNCC5)cccc4n3CC(F)(F)F)cc2)CC1)c1ccc2c(N)c(C(=O)N[C@H]3CCc4cc(N5C[C@H]6CC[C@@H](C5)N6)ccc4C3)sc2n1] |
| 60 | 890.12 | 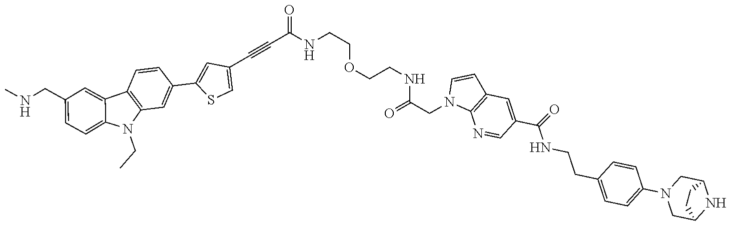 [SMILES: CCn1c2ccc(CNC)cc2c2ccc(-c3cc(C#CC(=O)NCCOCCNC(=O)Cn4ccc5cc(C(=O)NCCc6ccc(N7C[C@H]8CC[C@@H](C7)N8)cc6)cnc54)cs3)cc21] |
| 61 | 823.07 | 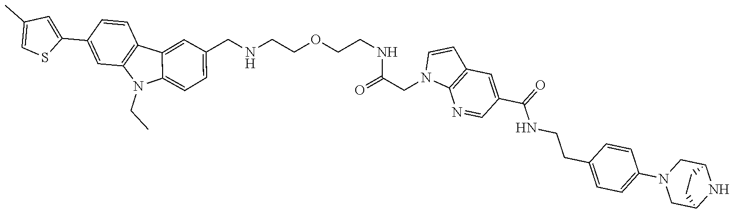 [SMILES: CCn1c2ccc(CNCCOCCNC(=O)Cn3ccc4cc(C(=O)NCCc5ccc(N6C[C@H]7CC[C@@H](C6)N7)cc5)cnc43)cc2c2ccc(-c3cc(C)cs3)cc21] |
| 62 | 1004.47 | 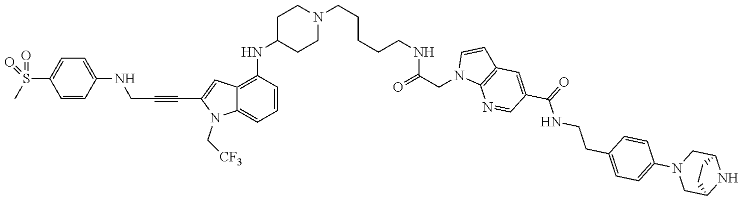 [SMILES: CS(=O)(=O)c1ccc(NCC#Cc2cc3c(NC4CCN(CCCCCNC(=O)Cn5ccc6cc(C(=O)NCCc7ccc(N8C[C@H]9CC[C@@H](C8)N9)cc7)cnc65)CC4)cccc3n2CC(F)(F)F)cc1] |
| 63 | 972.13 | 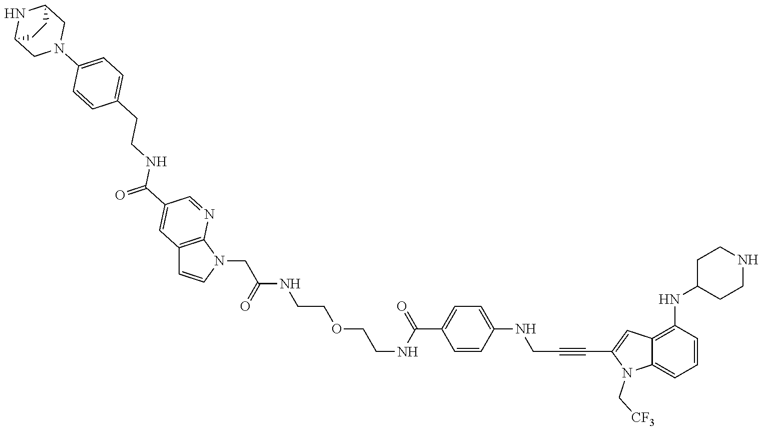 [SMILES: O=C(Cn1ccc2cc(C(=O)NCCc3ccc(N4C[C@H]5CC[C@@H](C4)N5)cc3)cnc21)NCCOCCNC(=O)c1ccc(NCC#Cc2cc3c(NC4CCNCC4)cccc3n2CC(F)(F)F)cc1] |
| 64 | 817.07 | 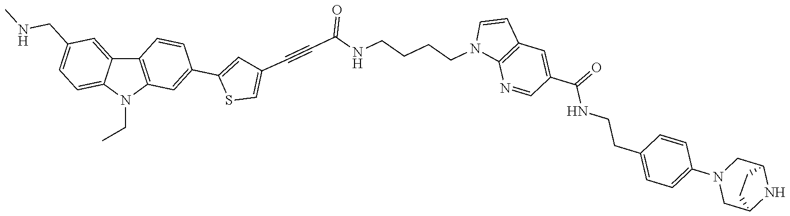 [SMILES: CCn1c2ccc(CNC)cc2c2ccc(-c3cc(C#CC(=O)NCCCCn4ccc5cc(C(=O)NCCc6ccc(N7C[C@H]8CC[C@@H](C7)N8)cc6)cnc54)cs3)cc21] |
| 65 | 934.14 | 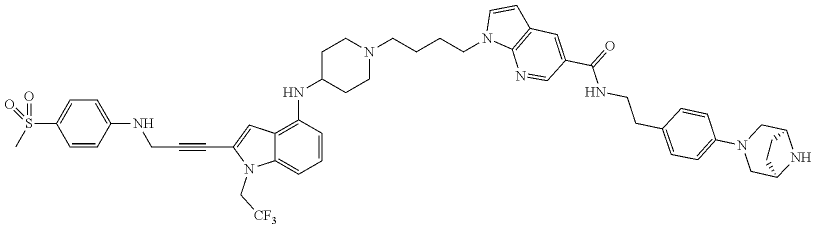 [SMILES: CS(=O)(=O)c1ccc(NCC#Cc2cc3c(NC4CCN(CCCCn5ccc6cc(C(=O)NCCc7ccc(N8C[C@H]9CC[C@@H](C8)N9)cc7)cnc65)CC4)cccc3n2CC(F)(F)F)cc1] |
| 66 | 899.08 | 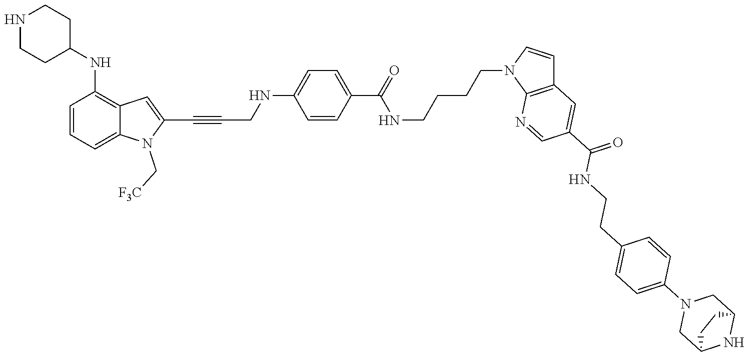 [SMILES: O=C(NCCCCn1ccc2cc(C(=O)NCCc3ccc(N4C[C@H]5CC[C@@H](C4)N5)cc3)cnc21)c1ccc(NCC#Cc2cc3c(NC4CCNCC4)cccc3n2CC(F)(F)F)cc1] |
| 67 | 838.15 | 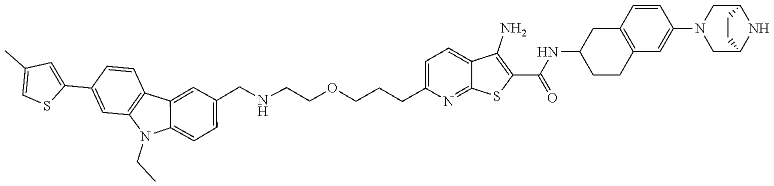 [SMILES: CCn1c2ccc(CNCCOCCCc3ccc4c(N)c(C(=O)NC5CCc6cc(N7C[C@H]8CC[C@@H](C7)N8)ccc6C5)sc4n3)cc2c2ccc(-c3cc(C)cs3)cc21] |
| 68 | 889.19 | 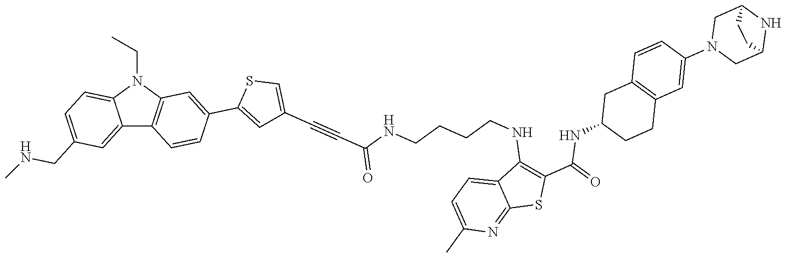 [SMILES: CCn1c2ccc(CNC)cc2c2ccc(-c3cc(C#CC(=O)NCCCCNc4c(C(=O)N[C@H]5CCc6cc(N7C[C@H]8CC[C@@H](C7)N8)ccc6C5)sc5nc(C)ccc45)cs3)cc21] |
| 69 | 822.15 | 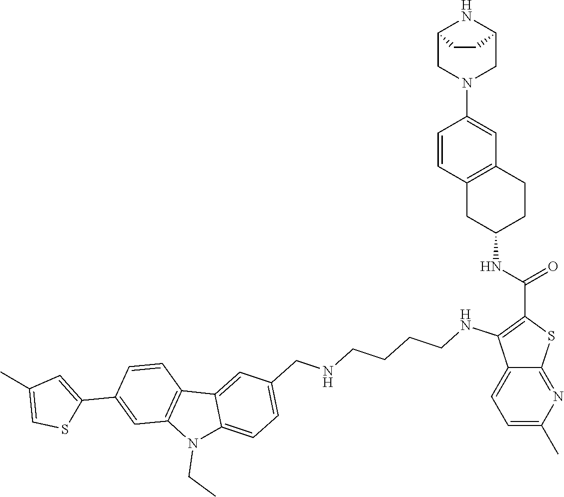 [SMILES: CCn1c2ccc(CNCCCCNc3c(C(=O)N[C@H]4CCc5cc(N6C[C@H]7CC[C@@H](C6)N7)ccc5C4)sc4nc(C)ccc34)cc2c2ccc(-c3cc(C)cs3)cc21] |
| 70 | 1006.27 | 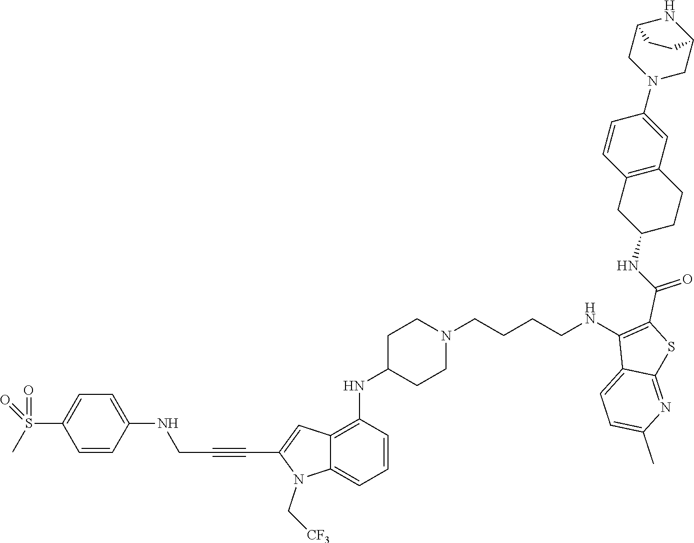 [SMILES: Cc1ccc2c(NCCCCN3CCC(Nc4cccc5c4cc(C#CCNc4ccc(S(C)(=O)=O)cc4)n5CC(F)(F)F)CC3)c(C(=O)N[C@H]3CCc4cc(N5C[C@H]6CC[C@@H](C5)N6)ccc4C3)sc2n1] |
| 71 | 971.21 | 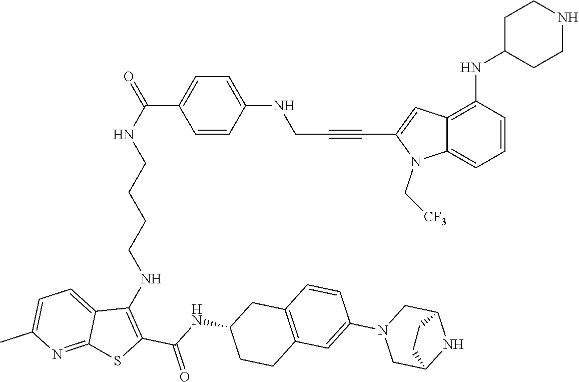 [SMILES: Cc1ccc2c(NCCCCNC(=O)c3ccc(NCC#Cc4cc5c(NC6CCNCC6)cccc5n4CC(F)(F)F)cc3)c(C(=O)N[C@H]3CCc4cc(N5C[C@H]6CC[C@@H](C5)N6)ccc4C3)sc2n1] |
| 72 | 868.09 | 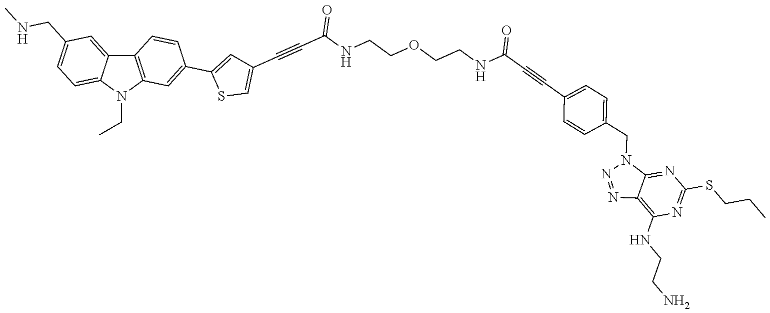 [SMILES: CCCSc1nc(NCCN)c2nnn(Cc3ccc(C#CC(=O)NCCOCCNC(=O)C#Cc4csc(-c5ccc6c7cc(CNC)ccc7n(CC)c6c5)c4)cc3)c2n1] |
| 73 | 876.16 | 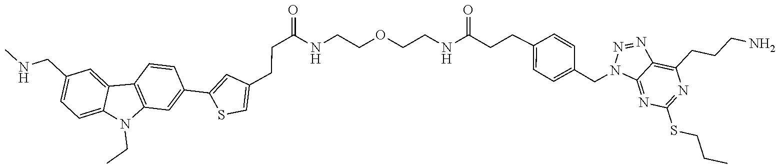 [SMILES: CCCSc1nc(CCCN)c2nnn(Cc3ccc(CCC(=O)NCCOCCNC(=O)CCc4csc(-c5ccc6c7cc(CNC)ccc7n(CC)c6c5)c4)cc3)c2n1] |
| 74 | 844.07 | 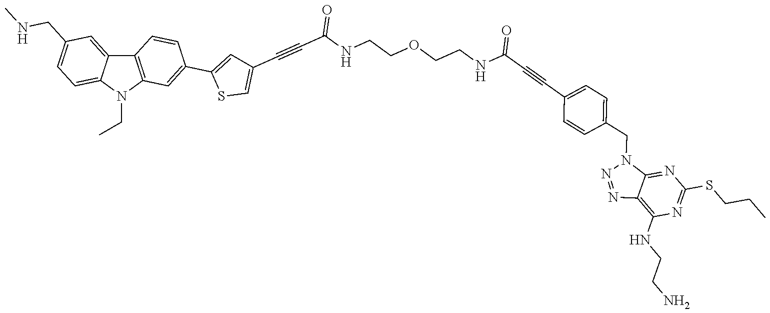 [SMILES: CCCSc1nc(NCCN)c2nnn(Cc3ccc(C#CC(=O)NCCOCCNC(=O)C#Cc4csc(-c5ccc6c7cc(CNC)ccc7n(CC)c6c5)c4)cc3)c2n1] |
| 75 | 848.10 | 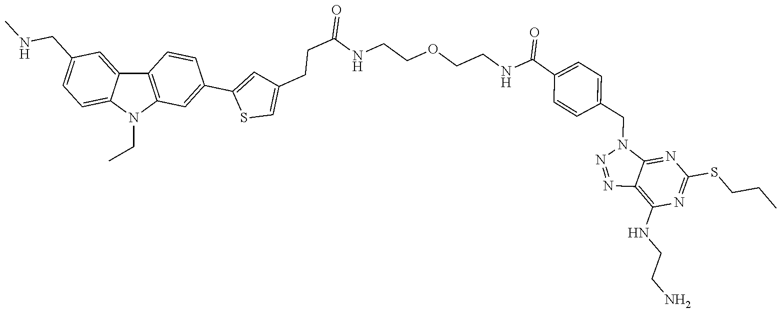 [SMILES: CCCSc1nc(NCCN)c2nnn(Cc3ccc(C(=O)NCCOCCNC(=O)CCc4csc(-c5ccc6c7cc(CNC)ccc7n(CC)c6c5)c4)cc3)c2n1] |
| 76 | 792.42 | 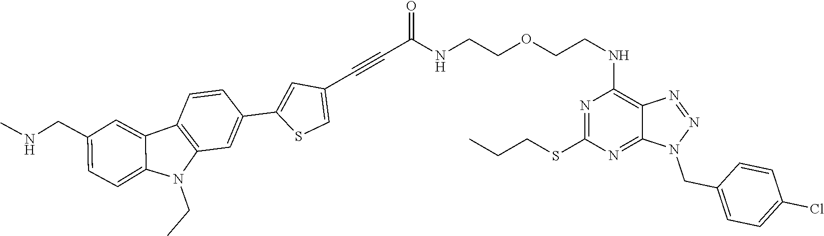 [SMILES: CCCSc1nc(NCCOCCNC(=O)C#Cc2csc(-c3ccc4c5cc(CNC)ccc5n(CC)c4c3)c2)c2nnn(Cc3ccc(Cl)cc3)c2n1] |
| 77 | 796.45 | 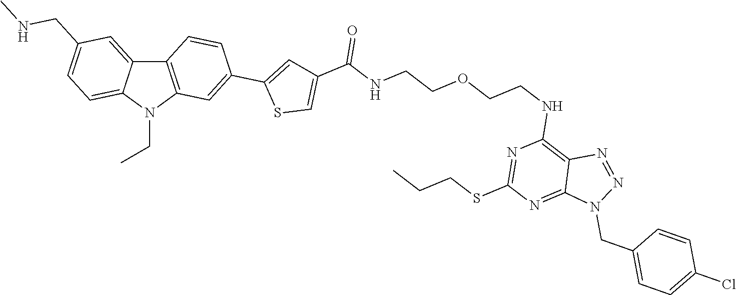 [SMILES: CCCSc1nc(NCCOCCNC(=O)c2csc(-c3ccc4c5cc(CNC)ccc5n(CC)c4c3)c2)c2nnn(Cc3ccc(Cl)cc3)c2n1] |
| 78 | 801.05 | 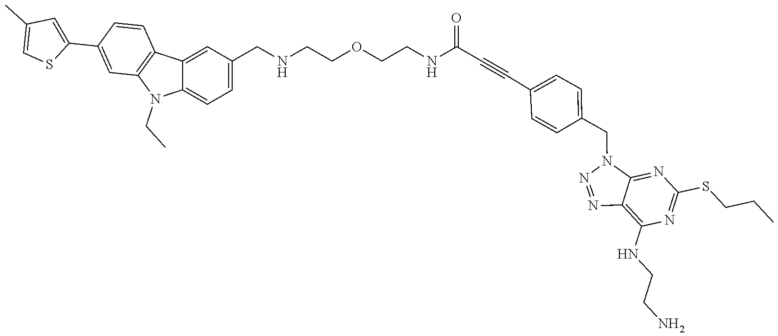 [SMILES: CCCSc1nc(NCCN)c2nnn(Cc3ccc(C#CC(=O)NCCOCCNCc4ccc5c(c4)c4ccc(-c6cc(C)cs6)cc4n5CC)cc3)c2n1] |
| 79 | 805.08 | 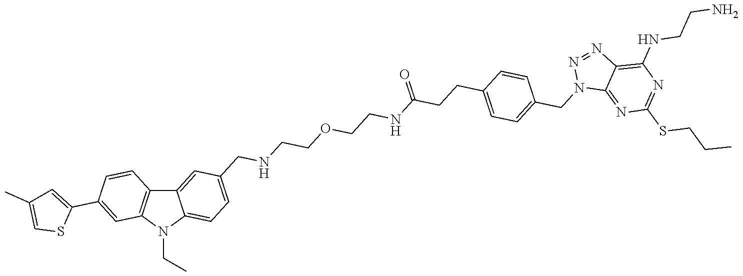 [SMILES: CCCSc1nc(NCCN)c2nnn(Cc3ccc(CCC(=O)NCCOCCNCc4ccc5c(c4)c4ccc(-c6cc(C)cs6)cc4n5CC)cc3)c2n1] |
| 80 | 725.37 | 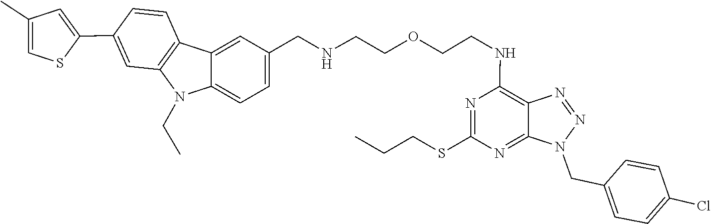 [SMILES: CCCSc1nc(NCCOCCNCc2ccc3c(c2)c2ccc(-c4cc(C)cs4)cc2n3CC)c2nnn(Cc3ccc(Cl)cc3)c2n1] |
| 81 | 983.19 | 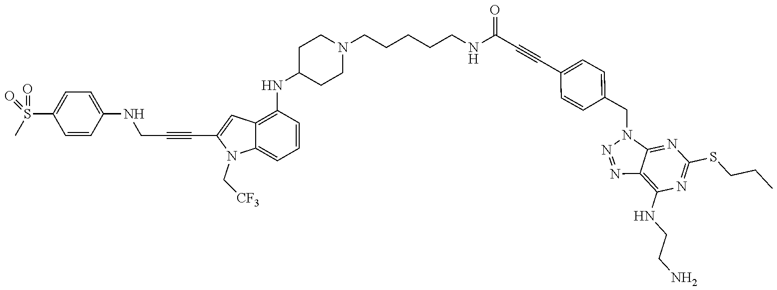 [SMILES: CCCSc1nc(NCCN)c2nnn(Cc3ccc(C#CC(=O)NCCCCCN4CCC(Nc5cccc6c5cc(C#CCNc5ccc(S(C)(=O)=O)cc5)n6CC(F)(F)F)CC4)cc3)c2n1] |
| 82 | 907.52 | 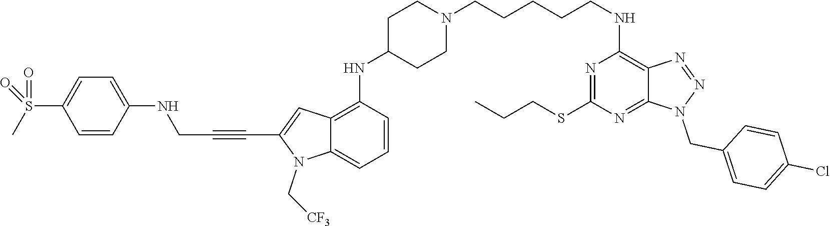 [SMILES: CCCSc1nc(NCCCCCN2CCC(Nc3cccc4c3cc(C#CCNc3ccc(S(C)(=O)=O)cc3)n4CC(F)(F)F)CC2)c2nnn(Cc3ccc(Cl)cc3)c2n1] |
| 83 | 950.10 | 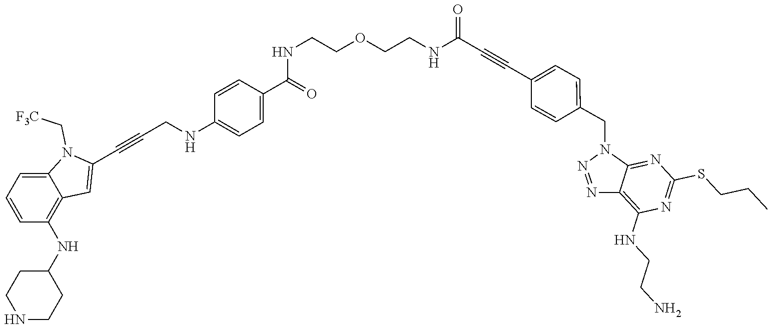 [SMILES: CCCSc1nc(NCCN)c2nnn(Cc3ccc(C#CC(=O)NCCOCCNC(=O)c4ccc(NCC#Cc5cc6c(NC7CCNCC7)cccc6n5CC(F)(F)F)cc4)cc3)c2n1] |
| 84 | 874.43 | 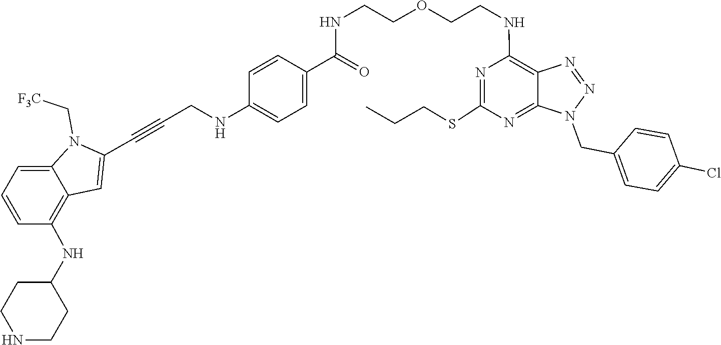 [SMILES: CCCSc1nc(NCCOCCNC(=O)c2ccc(NCC#Cc3cc4c(NC5CCNCC5)cccc4n3CC(F)(F)F)cc2)c2nnn(Cc3ccc(Cl)cc3)c2n1] |
| 85 | 921.20 | 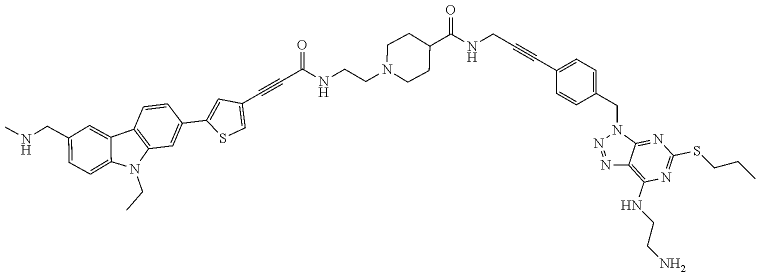 [SMILES: CCCSc1nc(NCCN)c2nnn(Cc3ccc(C#CCNC(=O)C4CCN(CCNC(=O)C#Cc5csc(-c6ccc7c8cc(CNC)ccc8n(CC)c7c6)c5)CC4)cc3)c2n1] |
| 86 | 929.26 | 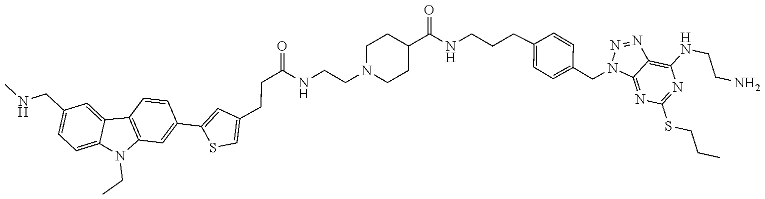 [SMILES: CCCSc1nc(NCCN)c2nnn(Cc3ccc(CCCNC(=O)C4CCN(CCNC(=O)CCc5csc(-c6ccc7c8cc(CNC)ccc8n(CC)c7c6)c5)CC4)cc3)c2n1] |
| 87 | 845.15 | 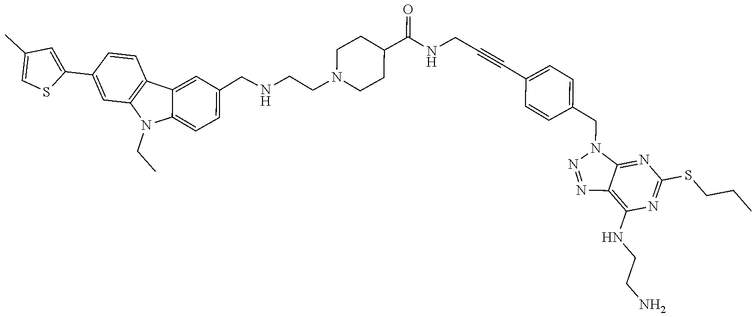 [SMILES: CCCSc1nc(NCCN)c2nnn(Cc3ccc(C#CCNC(=O)C4CCN(CCNCc5ccc6c(c5)c5ccc(-c7cc(C)cs7)cc5n6CC)CC4)cc3)c2n1] |
| 88 | 858.19 | 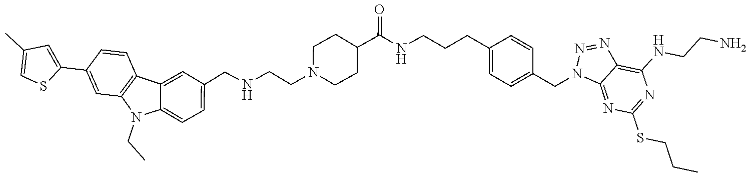 [SMILES: CCCSc1nc(NCCN)c2nnn(Cc3ccc(CCCNC(=O)C4CCN(CCNCc5ccc6c(c5)c5ccc(-c7cc(C)cs7)cc5n6CC)CC4)cc3)c2n1] |
| 89 | 1038.27 | 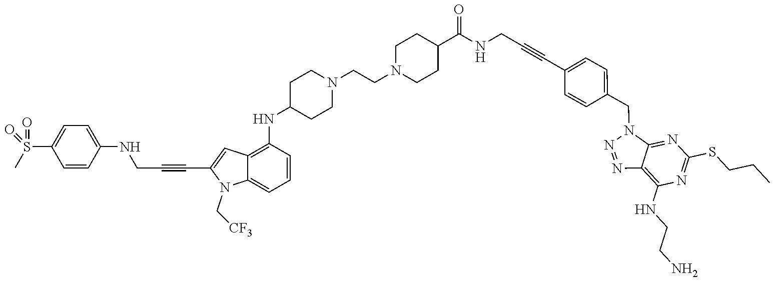 [SMILES: CCCSc1nc(NCCN)c2nnn(Cc3ccc(C#CCNC(=O)C4CCN(CCN5CCC(Nc6cccc7c6cc(C#CCNc6ccc(S(C)(=O)=O)cc6)n7CC(F)(F)F)CC5)CC4)cc3)c2n1] |
| 90 | 1003.21 | 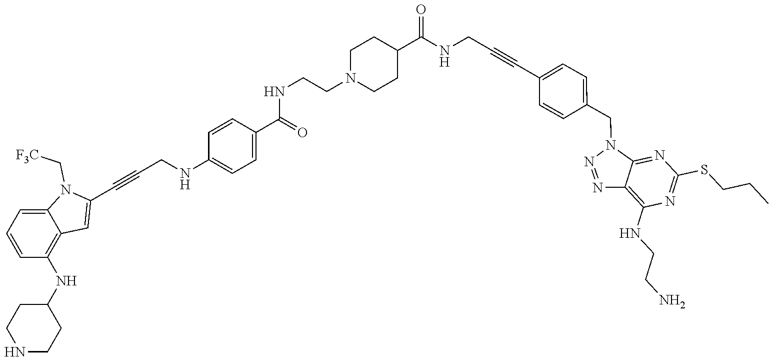 [SMILES: CCCSc1nc(NCCN)c2nnn(Cc3ccc(C#CCNC(=O)C4CCN(CCNC(=O)c5ccc(NCC#Cc6cc7c(NC8CCNCC8)cccc7n6CC(F)(F)F)cc5)CC4)cc3)c2n1] |
| 91 | 1016.22 | 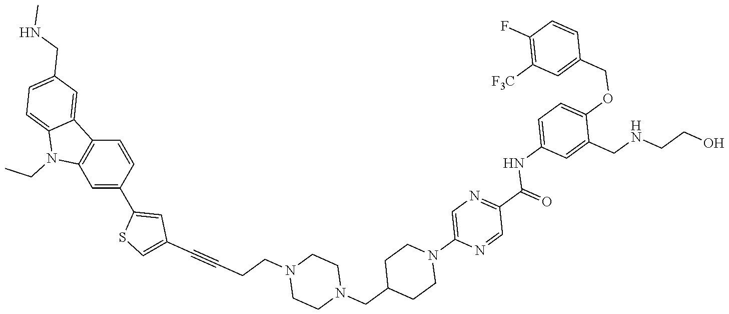 [SMILES: CCn1c2ccc(CNC)cc2c2ccc(-c3cc(C#CCCN4CCN(CC5CCN(c6cnc(C(=O)Nc7ccc(OCc8ccc(F)c(C(F)(F)F)c8)c(CNCCO)c7)cn6)CC5)CC4)cs3)cc21] |
| 92 | 1020.25 | 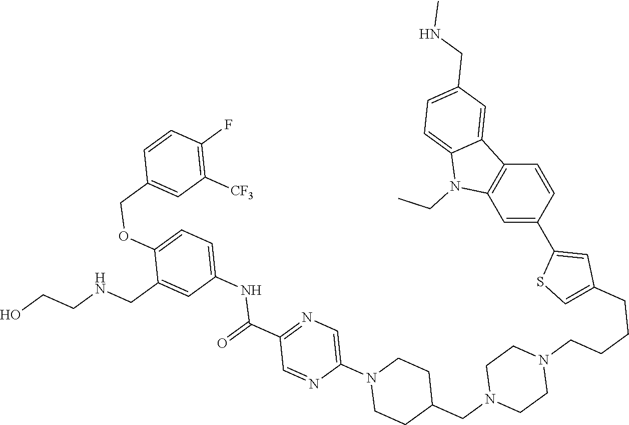 [SMILES: CCn1c2ccc(CNC)cc2c2ccc(-c3cc(CCCCN4CCN(CC5CCN(c6cnc(C(=O)Nc7ccc(OCc8ccc(F)c(C(F)(F)F)c8)c(CNCCO)c7)cn6)CC5)CC4)cs3)cc21] |
| 93 | 831.93 | 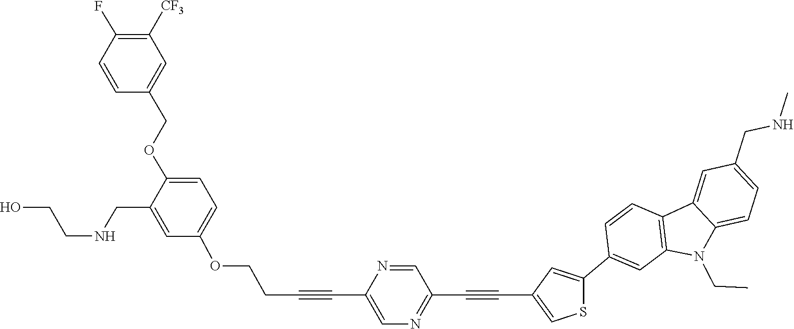 [SMILES: CCn1c2ccc(CNC)cc2c2ccc(-c3cc(C#Cc4cnc(C#CCCOc5ccc(OCc6ccc(F)c(C(F)(F)F)c6)c(CNCCO)c5)cn4)cs3)cc21] |
| 94 | 839.99 | 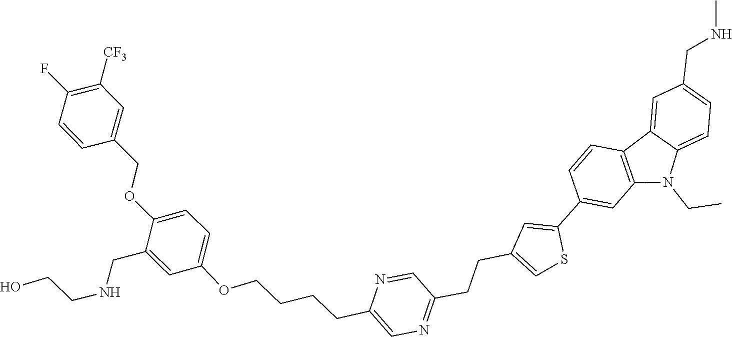 [SMILES: CCn1c2ccc(CNC)cc2c2ccc(-c3cc(CCc4cnc(CCCCOc5ccc(OCc6ccc(F)c(C(F)(F)F)c6)c(CNCCO)c5)cn4)cs3)cc21] |
| 95 | 814.94 | 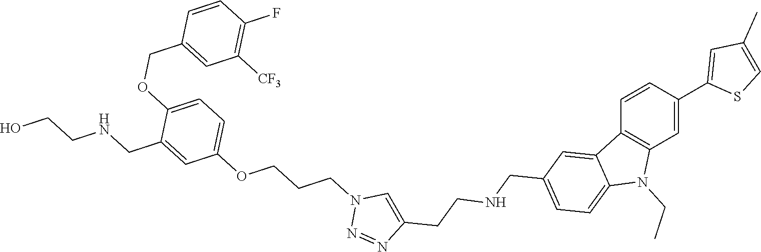 [SMILES: CCn1c2ccc(CNCCc3cn(CCCOc4ccc(OCc5ccc(F)c(C(F)(F)F)c5)c(CNCCO)c4)nn3)cc2c2ccc(-c3cc(C)cs3)cc21] |
| 96 | 785.90 | 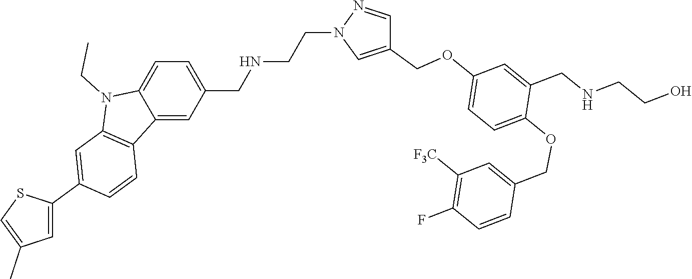 [SMILES: CCn1c2ccc(CNCCn3cc(COc4ccc(OCc5ccc(F)c(C(F)(F)F)c5)c(CNCCO)c4)cn3)cc2c2ccc(-c3cc(C)cs3)cc21] |
| 97 | 891.87 | 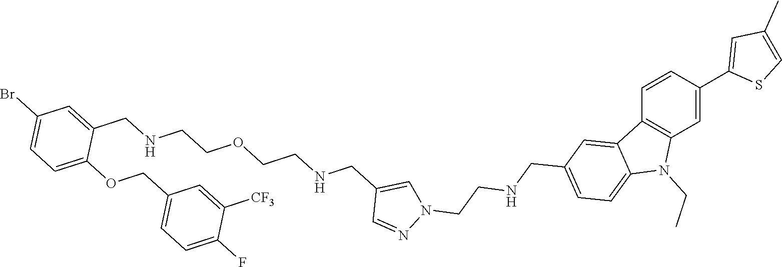 [SMILES: CCn1c2ccc(CNCCn3cc(CNCCOCCNCc4cc(Br)ccc4OCc4ccc(F)c(C(F)(F)F)c4)cn3)cc2c2ccc(-c3cc(C)cs3)cc21] |
| 98 | 859.99 | 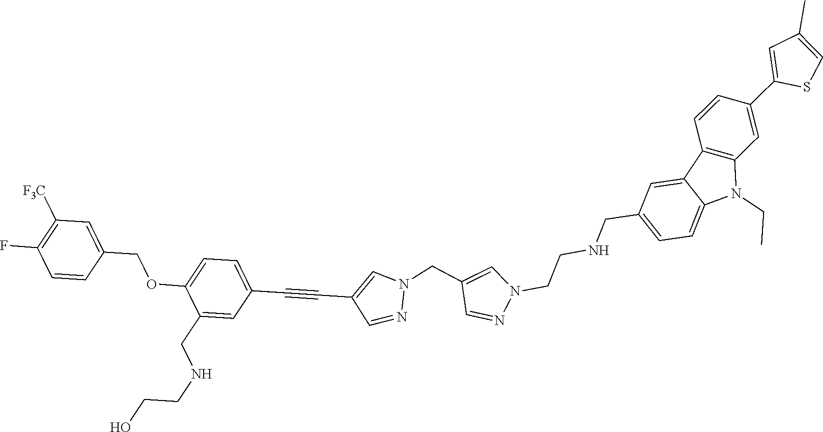 [SMILES: CCn1c2ccc(CNCCn3cc(Cn4cc(C#Cc5ccc(OCc6ccc(F)c(C(F)(F)F)c6)c(CNCCO)c5)cn4)cn3)cc2c2ccc(-c3cc(C)cs3)cc21] |
| 99 | 1011.16 | 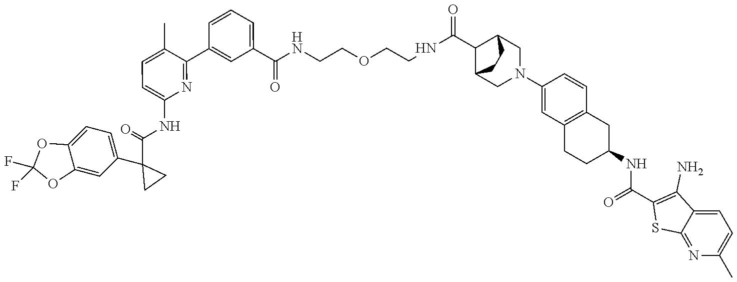 [SMILES: Cc1ccc2c(N)c(C(=O)N[C@H]3CCc4cc(N5C[C@H]6CC[C@@H](C5)C6C(=O)NCCOCCNC(=O)c5cccc(-c6nc(NC(=O)C7(c8ccc9c(c8)OC(F)(F)O9)CC7)ccc6C)c5)ccc4C3)sc2n1] |
| 100 | 1079.25 | 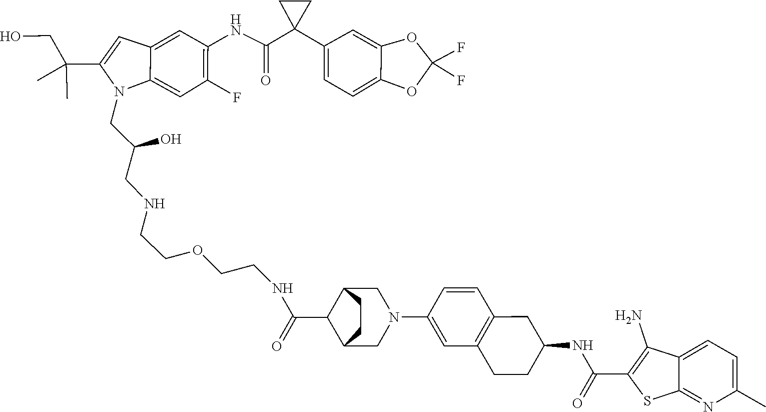 [SMILES: Cc1ccc2c(N)c(C(=O)N[C@H]3CCc4cc(N5C[C@H]6CC[C@@H](C5)C6C(=O)NCCOCCNC[C@H](O)Cn5c(C(C)(C)CO)cc6cc(NC(=O)C7(c8ccc9c(c8)OC(F)(F)O9)CC7)c(F)cc65)ccc4C3)sc2n1] |
| 101 | 1036.18 | 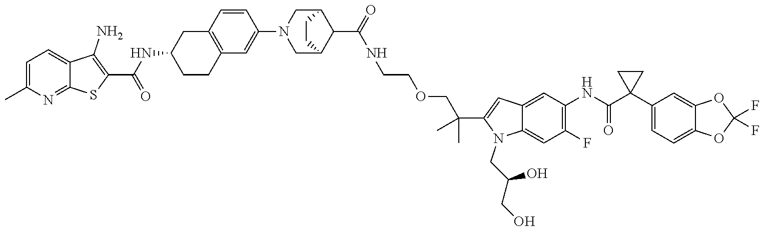 [SMILES: Cc1ccc2c(N)c(C(=O)N[C@H]3CCc4cc(N5C[C@H]6CC[C@@H](C5)C6C(=O)NCCOCC(C)(C)c5cc6cc(NC(=O)C7(c8ccc9c(c8)OC(F)(F)O9)CC7)c(F)cc6n5C[C@@H](O)CO)ccc4C3)sc2n1] |
| 102 | 1065.25 | 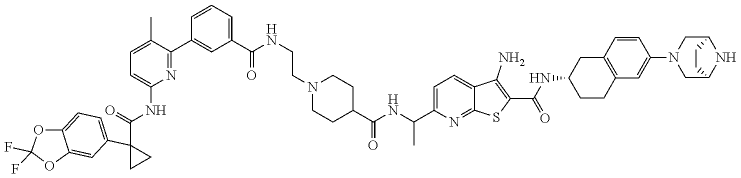 [SMILES: Cc1ccc(NC(=O)C2(c3ccc4c(c3)OC(F)(F)O4)CC2)nc1-c1cccc(C(=O)NCCN2CCC(C(=O)NC(C)c3ccc4c(N)c(C(=O)N[C@H]5CCc6cc(N7C[C@H]8CC[C@@H](C7)N8)ccc6C5)sc4n3)CC2)c1] |
| 103 | 1233.46 | 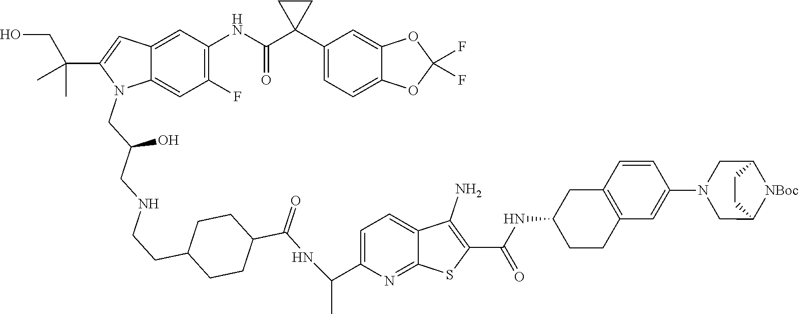 [SMILES: CC(NC(=O)C1CCC(CCNC[C@H](O)Cn2c(C(C)(C)CO)cc3cc(NC(=O)C4(c5ccc6c(c5)OC(F)(F)O6)CC4)c(F)cc32)CC1)c1ccc2c(N)c(C(=O)N[C@H]3CCc4cc(N5C[C@H]6CC[C@@H](C5)N6C(=O)OC(C)(C)C)ccc4C3)sc2n1] |
| 104 | 1134.33 | 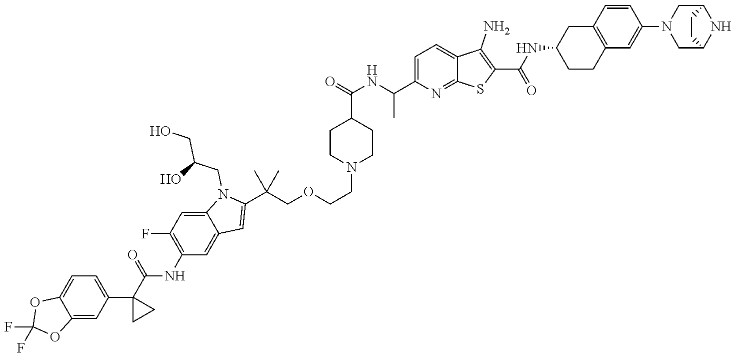 [SMILES: CC(NC(=O)C1CCN(CCOCC(C)(C)c2cc3cc(NC(=O)C4(c5ccc6c(c5)OC(F)(F)O6)CC4)c(F)cc3n2C[C@@H](O)CO)CC1)c1ccc2c(N)c(C(=O)N[C@H]3CCc4cc(N5C[C@H]6CC[C@@H](C5)N6)ccc4C3)sc2n1] |
| 105 | 954.05 | 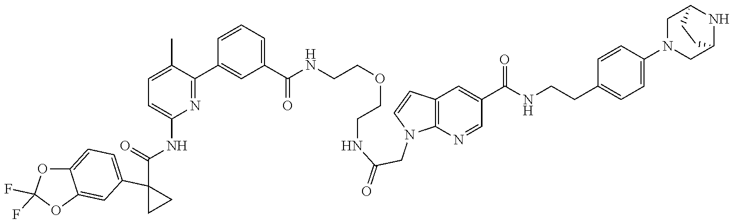 [SMILES: Cc1ccc(NC(=O)C2(c3ccc4c(c3)OC(F)(F)O4)CC2)nc1-c1cccc(C(=O)NCCOCCNC(=O)Cn2ccc3cc(C(=O)NCCc4ccc(N5C[C@H]6CC[C@@H](C5)N6)cc4)cnc32)c1] |
| 106 | 1022.14 | 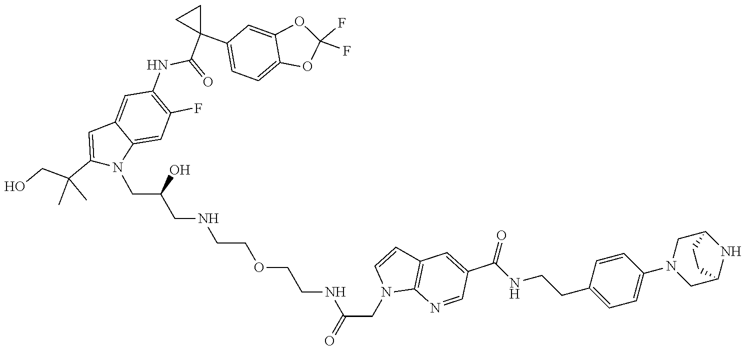 [SMILES: CC(C)(CO)c1cc2cc(NC(=O)C3(c4ccc5c(c4)OC(F)(F)O5)CC3)c(F)cc2n1C[C@@H](O)CNCCOCCNC(=O)Cn1ccc2cc(C(=O)NCCc3ccc(N4C[C@H]5CC[C@@H](C4)N5)cc3)cnc21] |
| 107 | 979.07 | 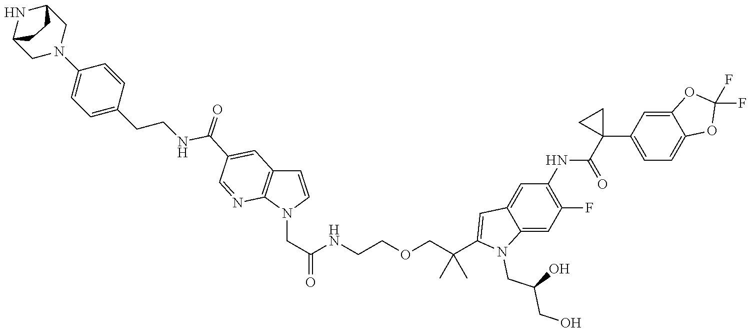 [SMILES: CC(C)(COCCNC(=O)Cn1ccc2cc(C(=O)NCCc3ccc(N4C[C@H]5CC[C@@H](C4)N5)cc3)cnc21)c1cc2cc(NC(=O)C3(c4ccc5c(c4)OC(F)(F)O5)CC3)c(F)cc2n1C[C@@H](O)CO] |
| 108 | 881.00 | 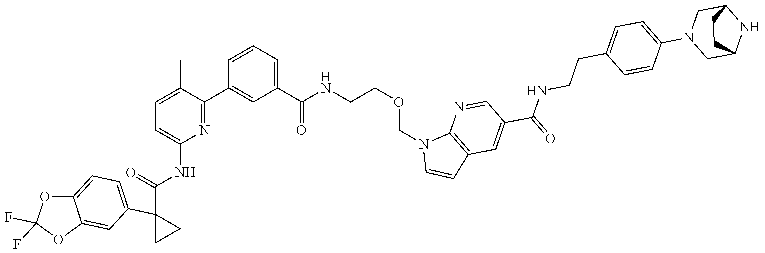 [SMILES: Cc1ccc(NC(=O)C2(c3ccc4c(c3)OC(F)(F)O4)CC2)nc1-c1cccc(C(=O)NCCOCn2ccc3cc(C(=O)NCCc4ccc(N5C[C@H]6CC[C@@H](C5)N6)cc4)cnc32)c1] |
| 109 | 969.12 | 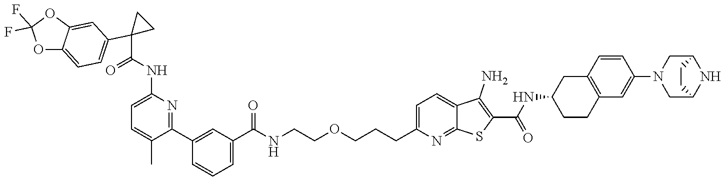 [SMILES: Cc1ccc(NC(=O)C2(c3ccc4c(c3)OC(F)(F)O4)CC2)nc1-c1cccc(C(=O)NCCOCCCc2ccc3c(N)c(C(=O)N[C@H]4CCc5cc(N6C[C@H]7CC[C@@H](C6)N7)ccc5C4)sc3n2)c1] |
| 110 | 1037.21 | 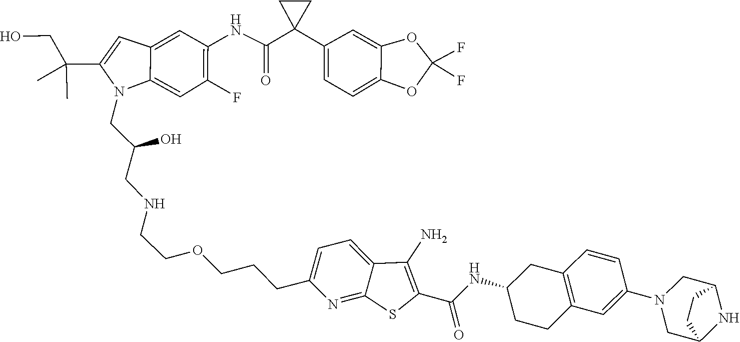 [SMILES: CC(C)(CO)c1cc2cc(NC(=O)C3(c4ccc5c(c4)OC(F)(F)O5)CC3)c(F)cc2n1C[C@@H](O)CNCCOCCCc1ccc2c(N)c(C(=O)N[C@H]3CCc4cc(N5C[C@H]6CC[C@@H](C5)N6)ccc4C3)sc2n1] |
| 111 | 953.12 | 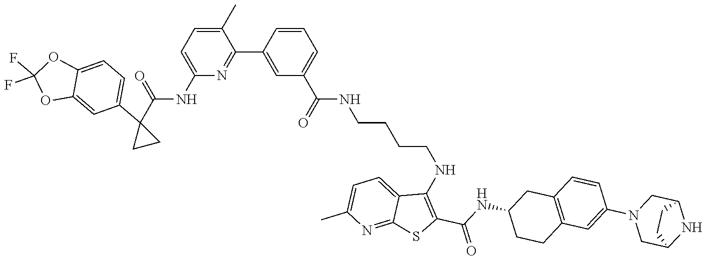 [SMILES: Cc1ccc2c(NCCCCNC(=O)c3cccc(-c4nc(NC(=O)C5(c6ccc7c(c6)OC(F)(F)O7)CC5)ccc4C)c3)c(C(=O)N[C@H]3CCc4cc(N5C[C@H]6CC[C@@H](C5)N6)ccc4C3)sc2n1] |
| 112 | 1021.21 | 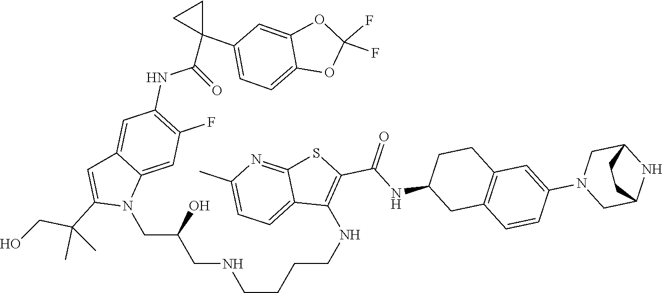 [SMILES: Cc1ccc2c(NCCCCNC[C@H](O)Cn3c(C(C)(C)CO)cc4cc(NC(=O)C5(c6ccc7c(c6)OC(F)(F)O7)CC5)c(F)cc43)c(C(=O)N[C@H]3CCc4cc(N5C[C@H]6CC[C@@H](C5)N6)ccc4C3)sc2n1] |
| 113 | 1022.20 | 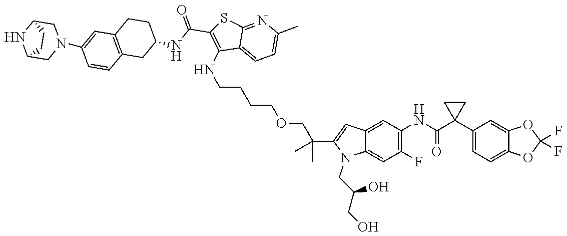 [SMILES: Cc1ccc2c(NCCCCOCC(C)(C)c3cc4cc(NC(=O)C5(c6ccc7c(c6)OC(F)(F)O7)CC5)c(F)cc4n3C[C@@H](O)CO)c(C(=O)N[C@H]3CCc4cc(N5C[C@H]6CC[C@@H](C5)N6)ccc4C3)sc2n1] |
| 114 | 932.02 | 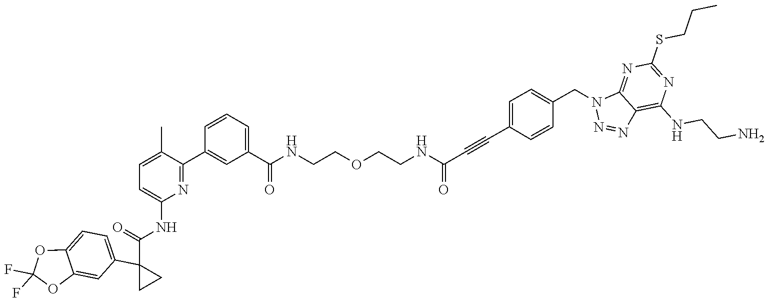 [SMILES: CCCSc1nc(NCCN)c2nnn(Cc3ccc(C#CC(=O)NCCOCCNC(=O)c4cccc(-c5nc(NC(=O)C6(c7ccc8c(c7)OC(F)(F)O8)CC6)ccc5C)c4)cc3)c2n1] |
| 115 | 936.05 | 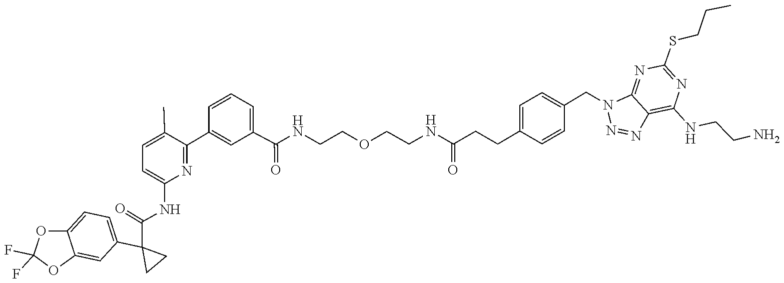 [SMILES: CCCSc1nc(NCCN)c2nnn(Cc3ccc(CCC(=O)NCCOCCNC(=O)c4cccc(-c5nc(NC(=O)C6(c7ccc8c(c7)OC(F)(F)O8)CC6)ccc5C)c4)cc3)c2n1] |
| 116 | 856.35 | 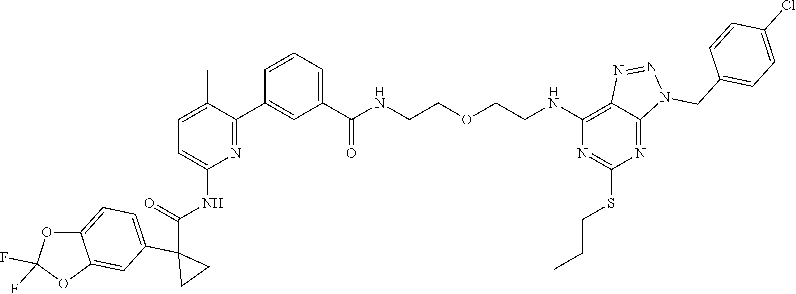 [SMILES: CCCSc1nc(NCCOCCNC(=O)c2cccc(-c3nc(NC(=O)C4(c5ccc6c(c5)OC(F)(F)O6)CC4)ccc3C)c2)c2nnn(Cc3ccc(Cl)cc3)c2n1] |
| 117 | 971.07 | 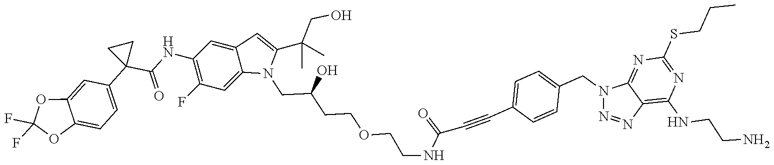 [SMILES: CCCSc1nc(NCCN)c2nnn(Cc3ccc(C#CC(=O)NCCOCC[C@H](O)Cn4c(C(C)(C)CO)cc5cc(NC(=O)C6(c7ccc8c(c7)OC(F)(F)O8)CC6)c(F)cc54)cc3)c2n1] |
| 118 | 924.44 | 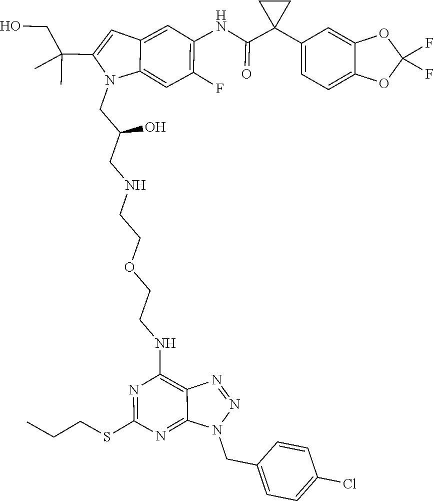 [SMILES: CCCSc1nc(NCCOCCNC[C@H](O)Cn2c(C(C)(C)CO)cc3cc(NC(=O)C4(c5ccc6c(c5)OC(F)(F)O6)CC4)c(F)cc32)c2nnn(Cc3ccc(Cl)cc3)c2n1] |
| 119 | 957.04 | 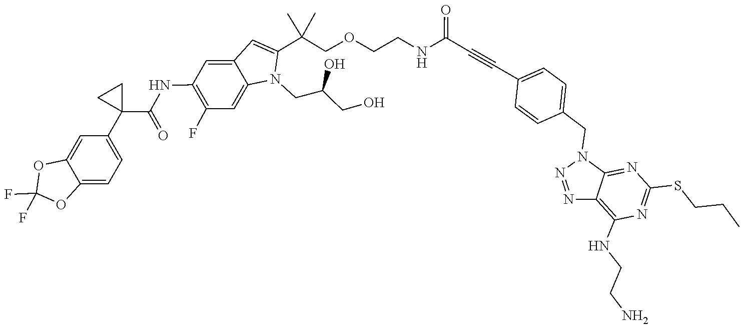 [SMILES: CCCSc1nc(NCCN)c2nnn(Cc3ccc(C#CC(=O)NCCOCC(C)(C)c4cc5cc(NC(=O)C6(c7ccc8c(c7)OC(F)(F)O8)CC6)c(F)cc5n4C[C@@H](O)CO)cc3)c2n1] |
| 120 | 881.37 | 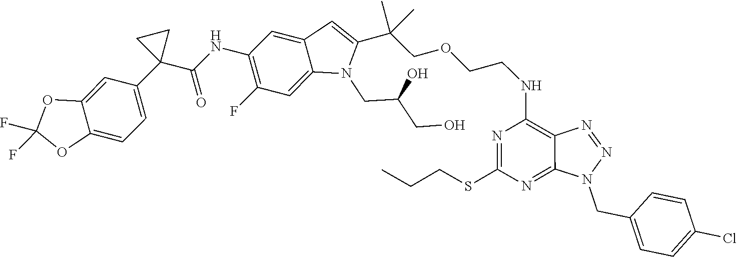 [SMILES: CCCSc1nc(NCCOCC(C)(C)c2cc3cc(NC(=O)C4(c5ccc6c(c5)OC(F)(F)O6)CC4)c(F)cc3n2C[C@@H](O)CO)c2nnn(Cc3ccc(Cl)cc3)c2n1] |
| 121 | 985.13 | 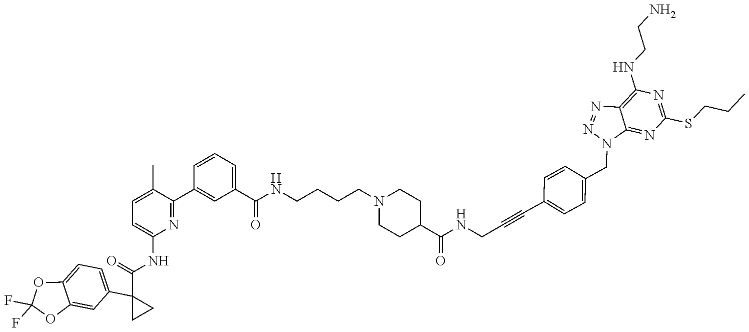 [SMILES: CCCSc1nc(NCCN)c2nnn(Cc3ccc(C#CCNC(=O)C4CCN(CCCCNC(=O)c5cccc(-c6nc(NC(=O)C7(c8ccc9c(c8)OC(F)(F)O9)CC7)ccc6C)c5)CC4)cc3)c2n1] |
| 122 | 989.16 | 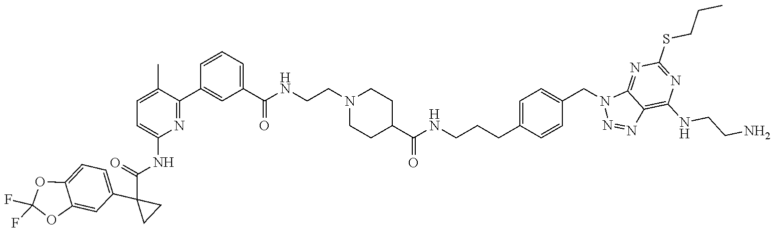 [SMILES: CCCSc1nc(NCCN)c2nnn(Cc3ccc(CCCNC(=O)C4CCN(CCNC(=O)c5cccc(-c6nc(NC(=O)C7(c8ccc9c(c8)OC(F)(F)O9)CC7)ccc6C)c5)CC4)cc3)c2n1] |
| 123 | 1053.22 | 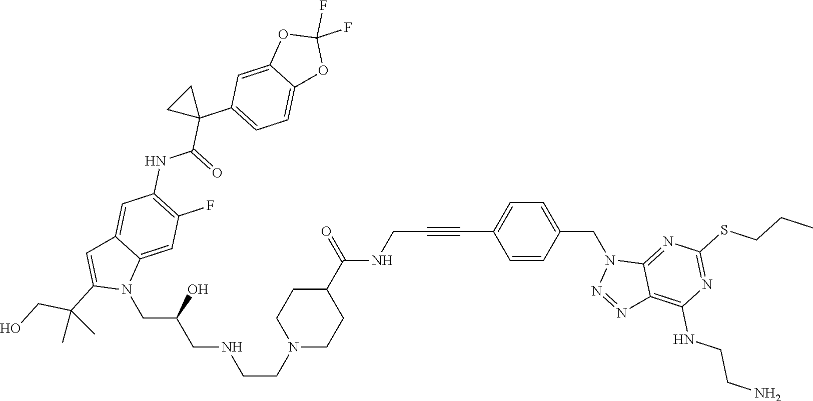 [SMILES: CCCSc1nc(NCCN)c2nnn(Cc3ccc(C#CCNC(=O)C4CCN(CCNC[C@H](O)Cn5c(C(C)(C)CO)cc6cc(NC(=O)C7(c8ccc9c(c8)OC(F)(F)O9)CC7)c(F)cc65)CC4)cc3)c2n1] |
| 124 | 1057.25 | 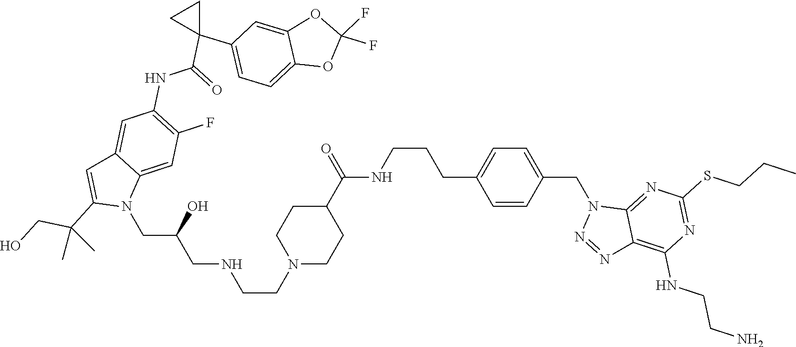 [SMILES: CCCSc1nc(NCCN)c2nnn(Cc3ccc(CCCNC(=O)C4CCN(CCNC[C@H](O)Cn5c(C(C)(C)CO)cc6cc(NC(=O)C7(c8ccc9c(c8)OC(F)(F)O9)CC7)c(F)cc65)CC4)cc3)c2n1] |
| 125 | 1054.20 | 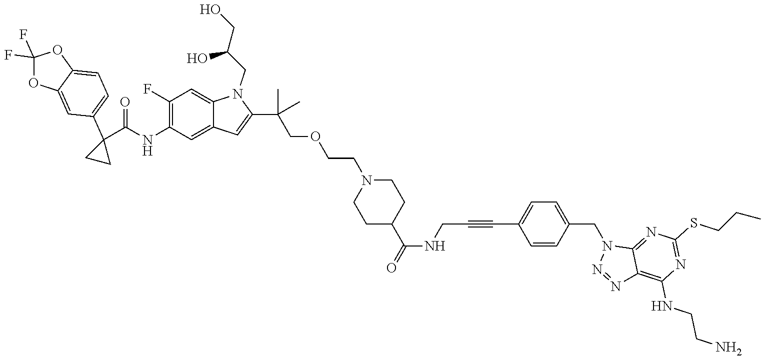 [SMILES: CCCSc1nc(NCCN)c2nnn(Cc3ccc(C#CCNC(=O)C4CCN(CCOCC(C)(C)c5cc6cc(NC(=O)C7(c8ccc9c(c8)OC(F)(F)O9)CC7)c(F)cc6n5C[C@@H](O)CO)CC4)cc3)c2n1] |
| 126 | 1058.24 | 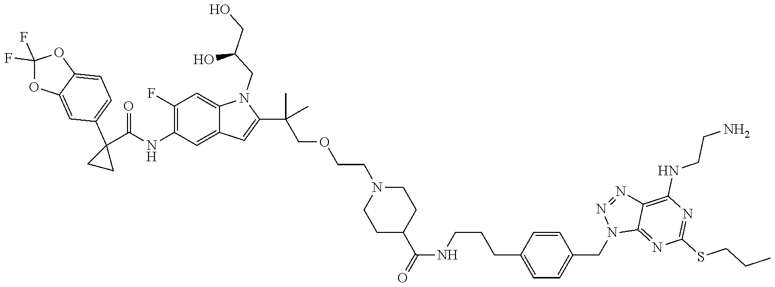 [SMILES: CCCSc1nc(NCCN)c2nnn(Cc3ccc(CCCNC(=O)C4CCN(CCOCC(C)(C)c5cc6cc(NC(=O)C7(c8ccc9c(c8)OC(F)(F)O9)CC7)c(F)cc6n5C[C@@H](O)CO)CC4)cc3)c2n1] |
| 127 | 976.93 | 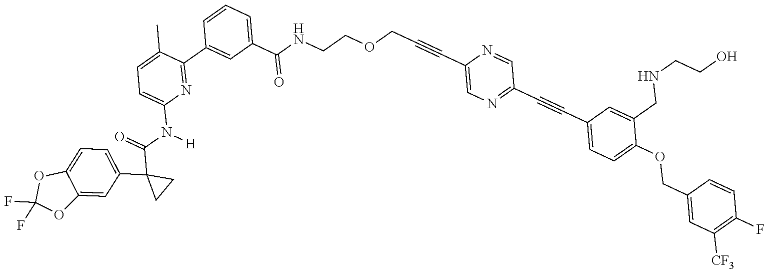 [SMILES: Cc1ccc(NC(=O)C2(c3ccc4c(c3)OC(F)(F)O4)CC2)nc1-c1cccc(C(=O)NCCOCC#Cc2cnc(C#Cc3ccc(OCc4ccc(F)c(C(F)(F)F)c4)c(CNCCO)c3)cn2)c1] |
| 128 | 985.00 | 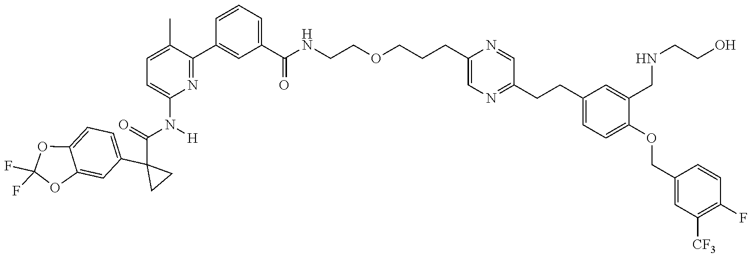 [SMILES: Cc1ccc(NC(=O)C2(c3ccc4c(c3)OC(F)(F)O4)CC2)nc1-c1cccc(C(=O)NCCOCCCc2cnc(CCc3ccc(OCc4ccc(F)c(C(F)(F)F)c4)c(CNCCO)c3)cn2)c1] |
| 129 | 992.98 | 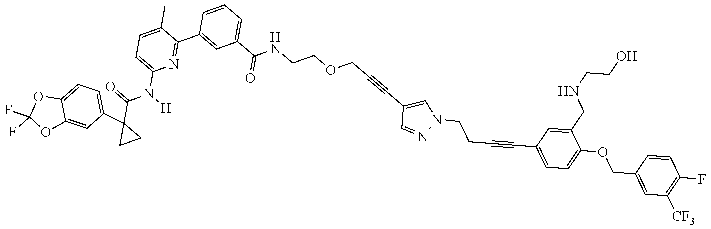 [SMILES: Cc1ccc(NC(=O)C2(c3ccc4c(c3)OC(F)(F)O4)CC2)nc1-c1cccc(C(=O)NCCOCC#Cc2cnn(CCC#Cc3ccc(OCc4ccc(F)c(C(F)(F)F)c4)c(CNCCO)c3)c2)c1] |
| 130 | 1001.04 | 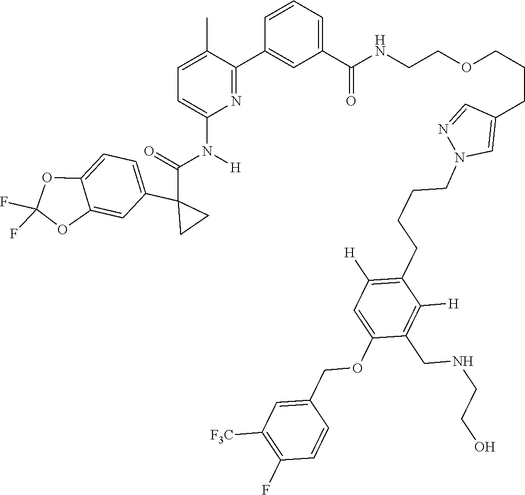 [SMILES: Cc1ccc(NC(=O)C2(c3ccc4c(c3)OC(F)(F)O4)CC2)nc1-c1cccc(C(=O)NCCOCCCc2cnn(CCCCc3ccc(OCc4ccc(F)c(C(F)(F)F)c4)c(CNCCO)c3)c2)c1] |
| 131 | 1215.24 | 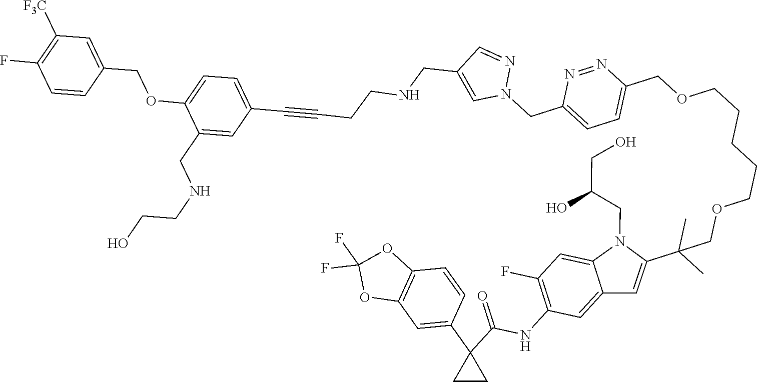 [SMILES: CC(C)(COCCCCCOCc1ccc(Cn2cc(CNCCC#Cc3ccc(OCc4ccc(F)c(C(F)(F)F)c4)c(CNCCO)c3)cn2)nn1)c1cc2cc(NC(=O)C3(c4ccc5c(c4)OC(F)(F)O5)CC3)c(F)cc2n1C[C@@H](O)CO] |
| 132 | 1219.27 | 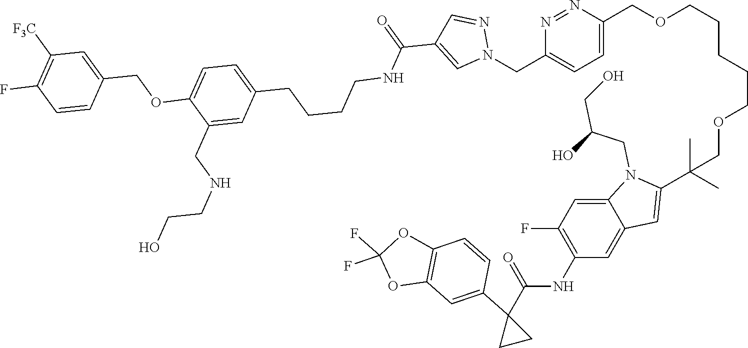 [SMILES: CC(C)(COCCCCCOCc1ccc(Cn2cc(C(=O)NCCCCc3ccc(OCc4ccc(F)c(C(F)(F)F)c4)c(CNCCO)c3)cn2)nn1)c1cc2cc(NC(=O)C3(c4ccc5c(c4)OC(F)(F)O5)CC3)c(F)cc2n1C[C@@H](O)CO] |
| 133 | 1270.11 | 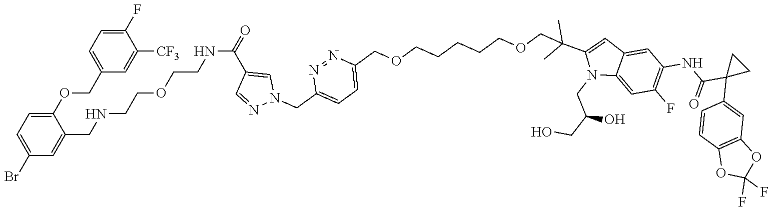 [SMILES: CC(C)(COCCCCCOCc1ccc(Cn2cc(C(=O)NCCOCCNCc3cc(Br)ccc3OCc3ccc(F)c(C(F)(F)F)c3)cn2)nn1)c1cc2cc(NC(=O)C3(c4ccc5c(c4)OC(F)(F)O5)CC3)c(F)cc2n1C[C@@H](O)CO] |
| 134 | 899.69 | 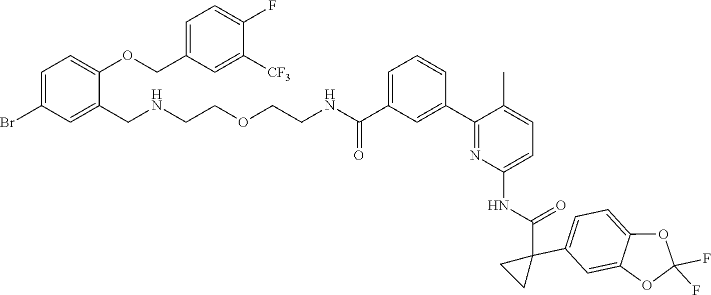 [SMILES: Cc1ccc(NC(=O)C2(c3ccc4c(c3)OC(F)(F)O4)CC2)nc1-c1cccc(C(=O)NCCOCCNCc2cc(Br)ccc2OCc2ccc(F)c(C(F)(F)F)c2)c1] |
| 135 | 1048.01 | 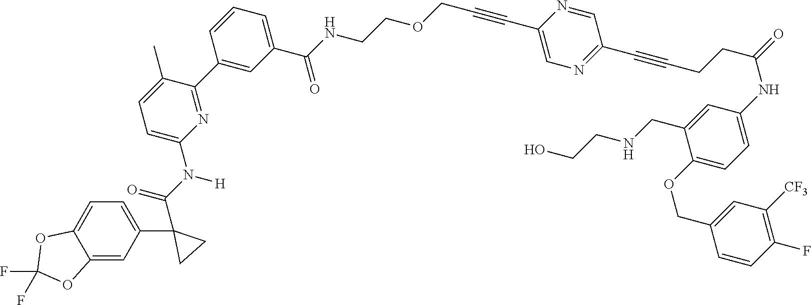 [SMILES: Cc1ccc(NC(=O)C2(c3ccc4c(c3)OC(F)(F)O4)CC2)nc1-c1cccc(C(=O)NCCOCC#Cc2cnc(C#CCCC(=O)Nc3ccc(OCc4ccc(F)c(C(F)(F)F)c4)c(CNCCO)c3)cn2)c1] |
| 136 | 1056.08 | 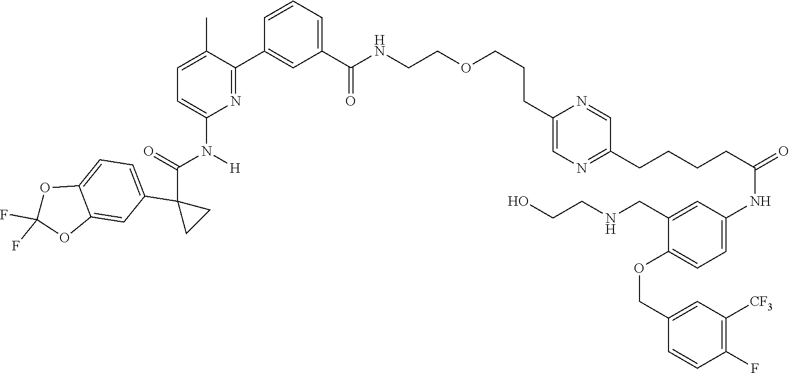 [SMILES: Cc1ccc(NC(=O)C2(c3ccc4c(c3)OC(F)(F)O4)CC2)nc1-c1cccc(C(=O)NCCOCCCc2cnc(CCCCC(=O)Nc3ccc(OCc4ccc(F)c(C(F)(F)F)c4)c(CNCCO)c3)cn2)c1] |
| 137 | 1012.98 | 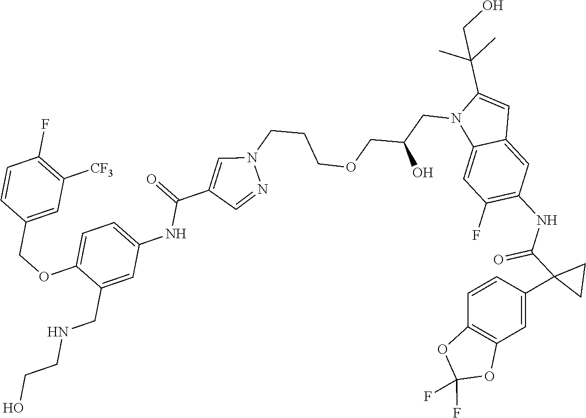 [SMILES: CC(C)(CO)c1cc2cc(NC(=O)C3(c4ccc5c(c4)OC(F)(F)O5)CC3)c(F)cc2n1C[C@@H](O)COCCCn1cc(C(=O)Nc2ccc(OCc3ccc(F)c(C(F)(F)F)c3)c(CNCCO)c2)cn1] |
| 138 | 948.15 | 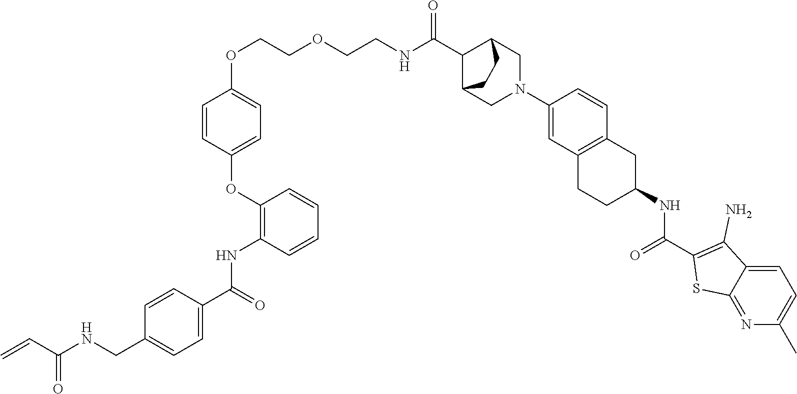 [SMILES: C=CC(=O)NCc1ccc(C(=O)Nc2ccccc2Oc2ccc(OCCOCCNC(=O)C3[C@@H]4CC[C@H]3CN(c3ccc5c(c3)CC[C@H](NC(=O)c3sc6nc(C)ccc6c3N)C5)C4)cc2)cc1] |
| 139 | 1002.25 | 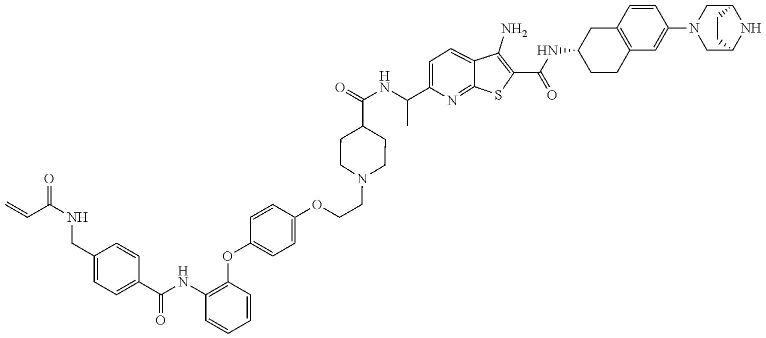 [SMILES: C=CC(=O)NCc1ccc(C(=O)Nc2ccccc2Oc2ccc(OCCN3CCC(C(=O)NC(C)c4ccc5c(N)c(C(=O)N[C@H]6CCc7cc(N8C[C@H]9CC[C@@H](C8)N9)ccc7C6)sc5n4)CC3)cc2)cc1] |
| 140 | 891.04 | 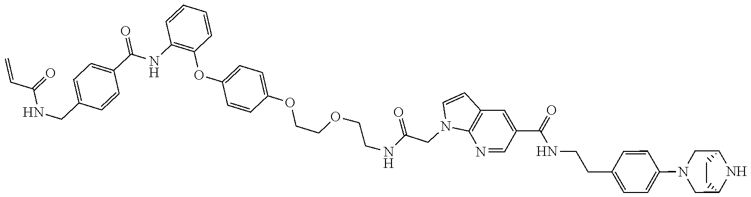 [SMILES: C=CC(=O)NCc1ccc(C(=O)Nc2ccccc2Oc2ccc(OCCOCCNC(=O)Cn3ccc4cc(C(=O)NCCc5ccc(N6C[C@H]7CC[C@@H](C6)N7)cc5)cnc43)cc2)cc1] |
| 141 | 817.09 | 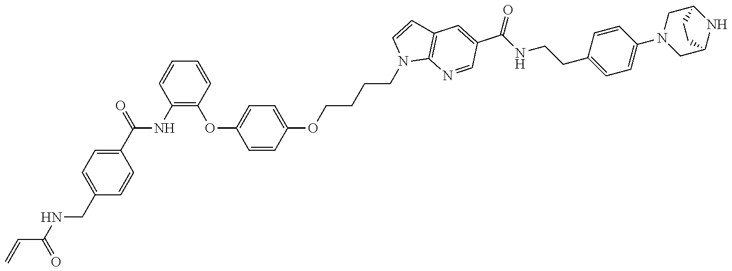 [SMILES: C=CC(=O)NCc1ccc(C(=O)Nc2ccccc2Oc2ccc(OCCCCn3ccc4cc(C(=O)NCCc5ccc(N6C[C@H]7CC[C@@H](C6)N7)cc5)cnc43)cc2)cc1] |
| 142 | 974.24 | 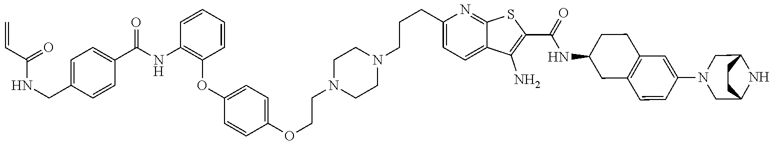 [SMILES: C=CC(=O)NCc1ccc(C(=O)Nc2ccccc2Oc2ccc(OCCN3CCN(CCCc4ccc5c(N)c(C(=O)N[C@H]6CCc7cc(N8C[C@H]9CC[C@@H](C8)N9)ccc7C6)sc5n4)CC3)cc2)cc1] |
| 143 | 890.12 | 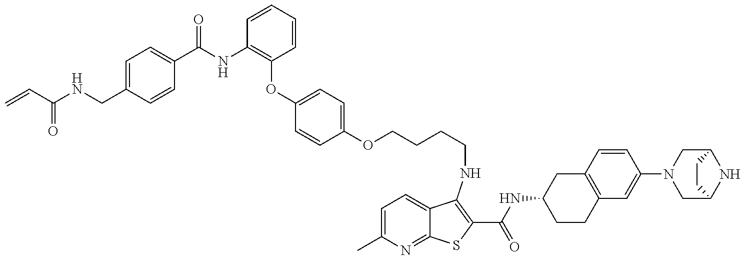 [SMILES: C=CC(=O)NCc1ccc(C(=O)Nc2ccccc2Oc2ccc(OCCCCNc3c(C(=O)N[C@H]4CCc5cc(N6C[C@H]7CC[C@@H](C6)N7)ccc5C4)sc4nc(C)ccc34)cc2)cc1] |
| 144 | 868.03 | 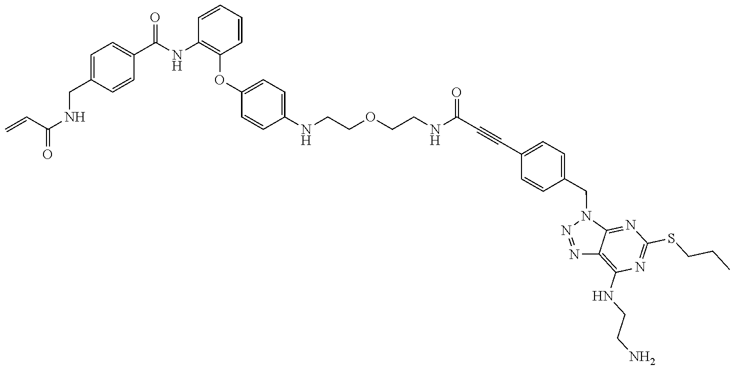 [SMILES: C=CC(=O)NCc1ccc(C(=O)Nc2ccccc2Oc2ccc(NCCOCCNC(=O)C#Cc3ccc(Cn4nnc5c(NCCN)nc(SCCC)nc54)cc3)cc2)cc1] |
| 145 | 793.34 | 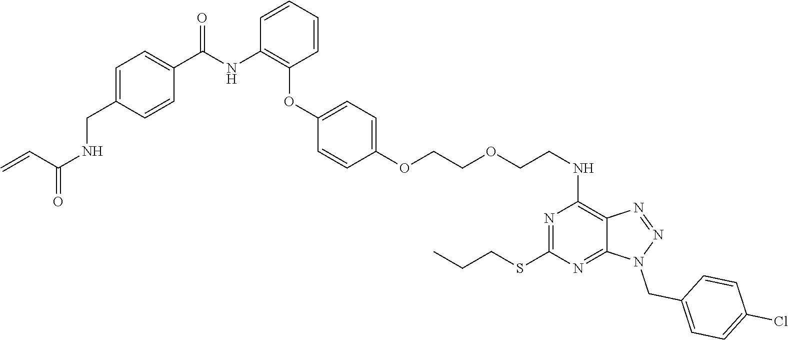 [SMILES: C=CC(=O)NCc1ccc(C(=O)Nc2ccccc2Oc2ccc(OCCOCCNc3nc(SCCC)nc4c3nnn4Cc3ccc(Cl)cc3)cc2)cc1] |
| 146 | 869.02 | 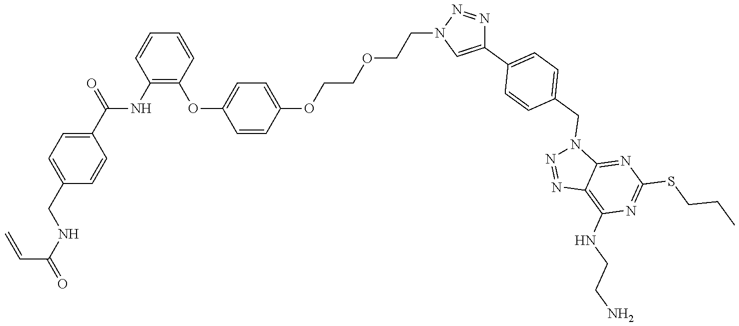 [SMILES: C=CC(=O)NCc1ccc(C(=O)Nc2ccccc2Oc2ccc(OCCOCCn3cc(-c4ccc(Cn5nnc6c(NCCN)nc(SCCC)nc65)cc4)nn3)cc2)cc1] |
| 147 | 875.41 | 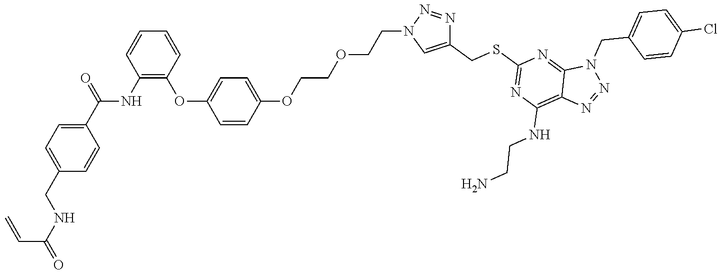 [SMILES: C=CC(=O)NCc1ccc(C(=O)Nc2ccccc2Oc2ccc(OCCOCCn3cc(CSc4nc(NCCN)c5nnn(Cc6ccc(Cl)cc6)c5n4)nn3)cc2)cc1] |
| 148 | 918.94 | 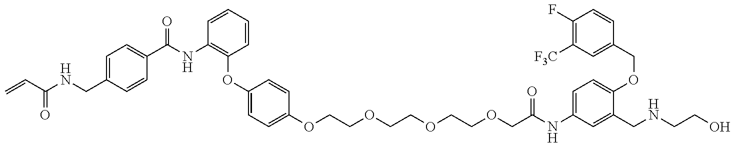 [SMILES: C=CC(=O)NCc1ccc(C(=O)Nc2ccccc2Oc2ccc(OCCOCCOCCOCC(=O)Nc3ccc(OCc4ccc(F)c(C(F)(F)F)c4)c(CNCCO)c3)cc2)cc1] |
| 149 | 971.02 | 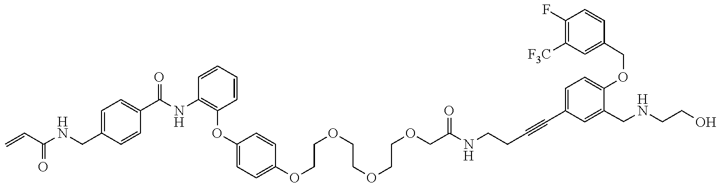 [SMILES: C=CC(=O)NCc1ccc(C(=O)Nc2ccccc2Oc2ccc(OCCOCCOCCOCC(=O)NCCC#Cc3ccc(OCc4ccc(F)c(C(F)(F)F)c4)c(CNCCO)c3)cc2)cc1] |
| 150 | 975.05 | 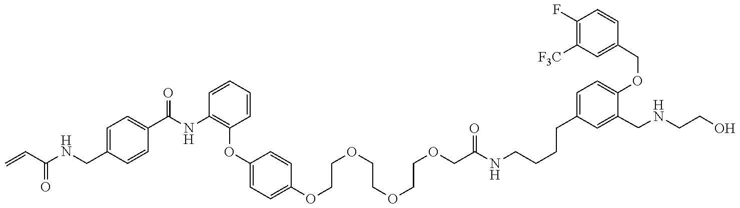 [SMILES: C=CC(=O)NCc1ccc(C(=O)Nc2ccccc2Oc2ccc(OCCOCCOCCOCC(=O)NCCCCc3ccc(OCc4ccc(F)c(C(F)(F)F)c4)c(CNCCO)c3)cc2)cc1] |
| 151 | 1012.66 | 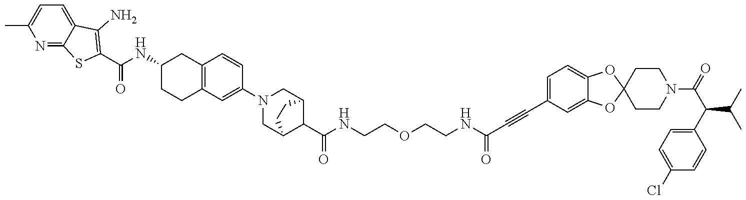 [SMILES: Cc1ccc2c(N)c(C(=O)N[C@H]3CCc4cc(N5C[C@H]6CC[C@@H](C5)C6C(=O)NCCOCCNC(=O)C#Cc5ccc6c(c5)OC5(CCN(C(=O)[C@H](c7ccc(Cl)cc7)C(C)C)CC5)O6)ccc4C3)sc2n1] |
| 152 | 1066.76 | 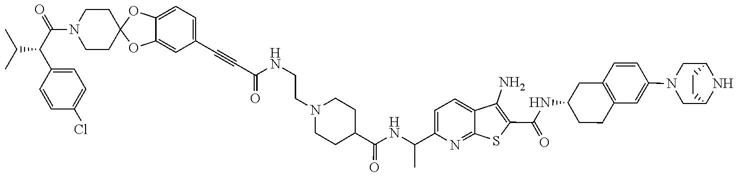 [SMILES: CC(NC(=O)C1CCN(CCNC(=O)C#Cc2ccc3c(c2)OC2(CCN(C(=O)[C@H](c4ccc(Cl)cc4)C(C)C)CC2)O3)CC1)c1ccc2c(N)c(C(=O)N[C@H]3CCc4cc(N5C[C@H]6CC[C@@H](C5)N6)ccc4C3)sc2n1] |
| 153 | 955.55 | 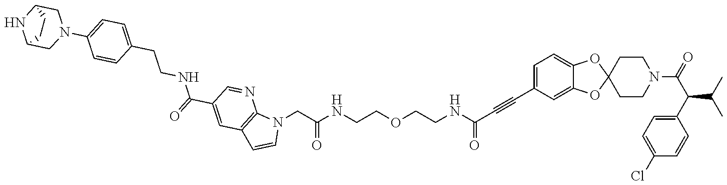 [SMILES: CC(C)[C@H](C(=O)N1CCC2(CC1)Oc1ccc(C#CC(=O)NCCOCCNC(=O)Cn3ccc4cc(C(=O)NCCc5ccc(N6C[C@H]7CC[C@@H](C6)N7)cc5)cnc43)cc1O2)c1ccc(Cl)cc1] |
| 154 | 882.50 | 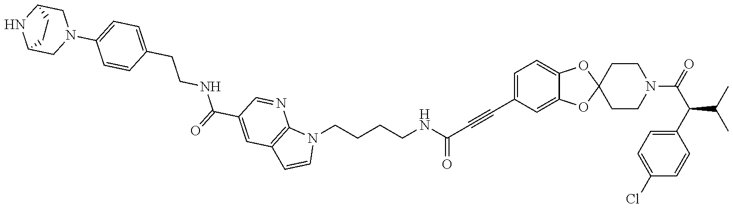 [SMILES: CC(C)[C@H](C(=O)N1CCC2(CC1)Oc1ccc(C#CC(=O)NCCCCn3ccc4cc(C(=O)NCCc5ccc(N6C[C@H]7CC[C@@H](C6)N7)cc5)cnc43)cc1O2)c1ccc(Cl)cc1] |
| 155 | 954.63 | 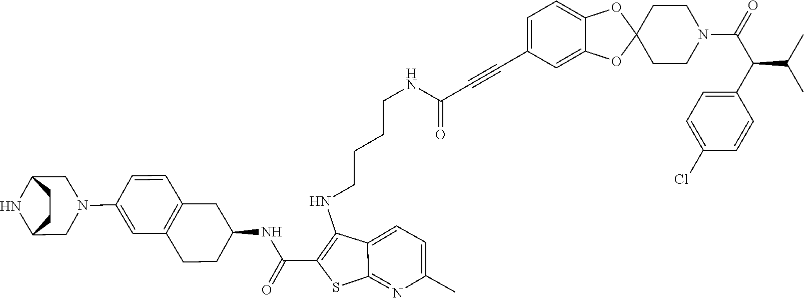 [SMILES: Cc1ccc2c(NCCCCNC(=O)C#Cc3ccc4c(c3)OC3(CCN(C(=O)[C@H](c5ccc(Cl)cc5)C(C)C)CC3)O4)c(C(=O)N[C@H]3CCc4cc(N5C[C@H]6CC[C@@H](C5)N6)ccc4C3)sc2n1] |
| 156 | 933.53 | 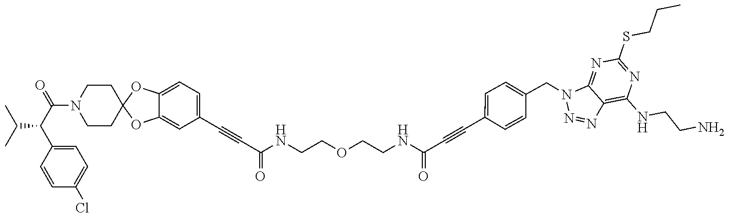 [SMILES: CCCSc1nc(NCCN)c2nnn(Cc3ccc(C#CC(=O)NCCOCCNC(=O)C#Cc4ccc5c(c4)OC4(CCN(C(=O)[C@H](c6ccc(Cl)cc6)C(C)C)CC4)O5)cc3)c2n1] |
| 157 | 857.85 | 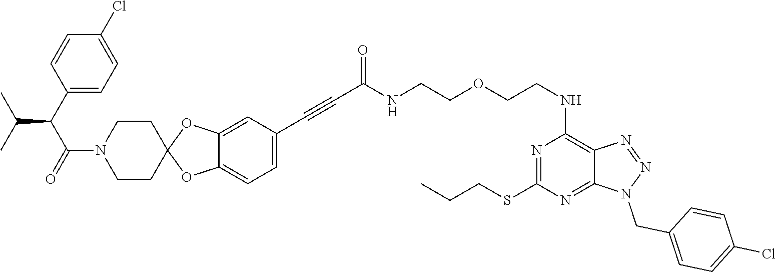 [SMILES: CCCSc1nc(NCCOCCNC(=O)C#Cc2ccc3c(c2)OC2(CCN(C(=O)[C@H](c4ccc(Cl)cc4)C(C)C)CC2)O3)c2nnn(Cc3ccc(Cl)cc3)c2n1] |
| 158 | 986.63 | 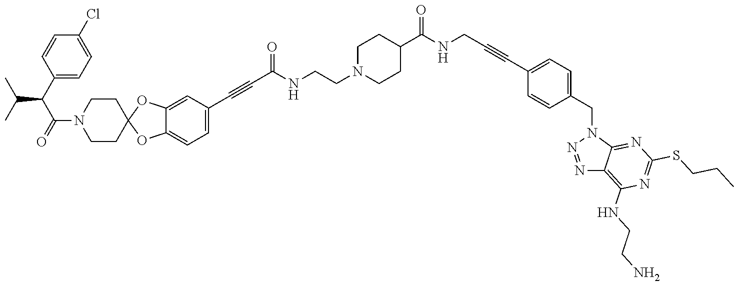 [SMILES: CCCSc1nc(NCCN)c2nnn(Cc3ccc(C#CCNC(=O)C4CCN(CCNC(=O)C#Cc5ccc6c(c5)OC5(CCN(C(=O)[C@H](c7ccc(Cl)cc7)C(C)C)CC5)O6)CC4)cc3)c2n1] |
| 159 | 933.53 | 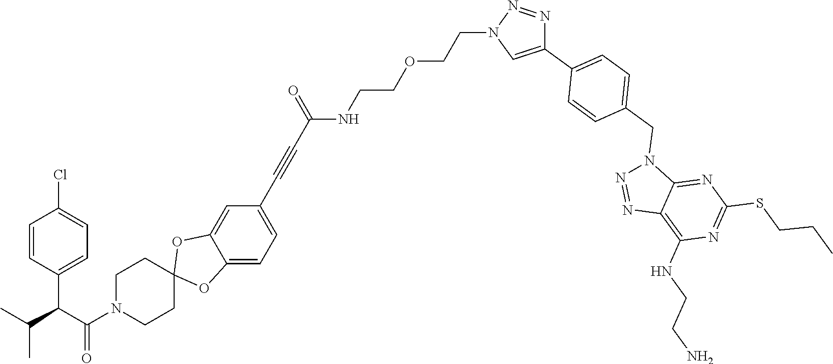 [SMILES: CCCSc1nc(NCCN)c2nnn(Cc3ccc(-c4cn(CCOCCNC(=O)C#Cc5ccc6c(c5)OC5(CCN(C(=O)[C@H](c7ccc(Cl)cc7)C(C)C)CC5)O6)nn4)cc3)c2n1] |
| 160 | 939.92 | 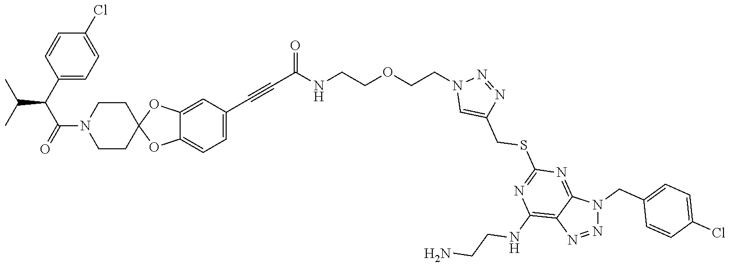 [SMILES: CC(C)[C@H](C(=O)N1CCC2(CC1)Oc1ccc(C#CC(=O)NCCOCCn3cc(CSc4nc(NCCN)c5nnn(Cc6ccc(Cl)cc6)c5n4)nn3)cc1O2)c1ccc(Cl)cc1] |
| 161 | 869.35 | 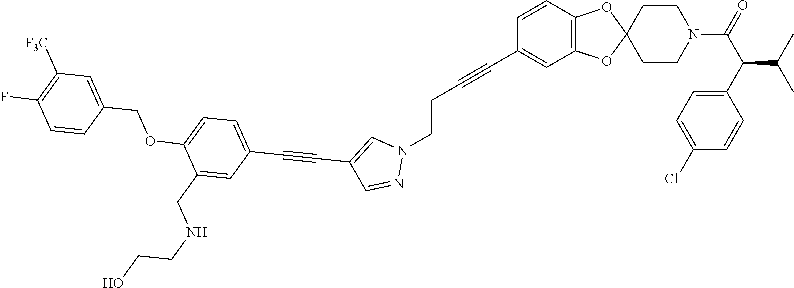 [SMILES: CC(C)[C@H](C(=O)N1CCC2(CC1)Oc1ccc(C#CCCn3cc(C#Cc4ccc(OCc5ccc(F)c(C(F)(F)F)c5)c(CNCCO)c4)cn3)cc1O2)c1ccc(Cl)cc1] |
| 162 | 877.42 | 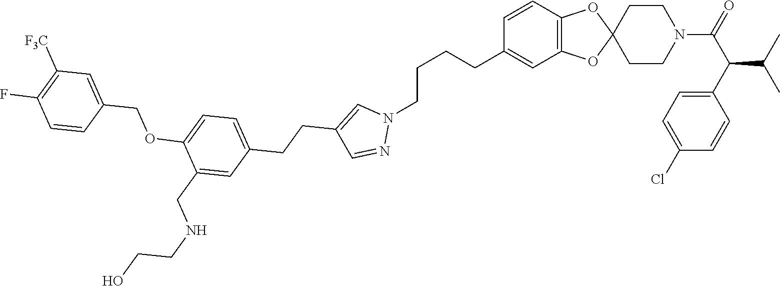 [SMILES: CC(C)[C@H](C(=O)N1CCC2(CC1)Oc1ccc(CCCCn3cc(CCc4ccc(OCc5ccc(F)c(C(F)(F)F)c5)c(CNCCO)c4)cn3)cc1O2)c1ccc(Cl)cc1] |
| 163 | 869.35 | 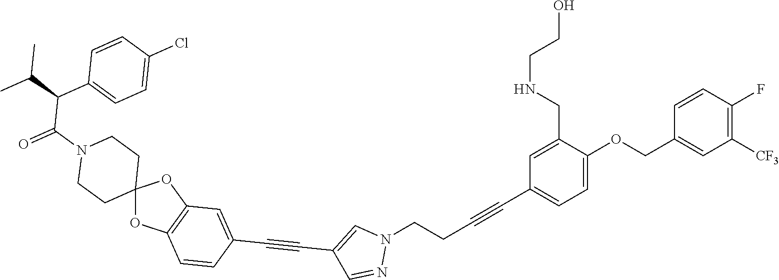 [SMILES: CC(C)[C@H](C(=O)N1CCC2(CC1)Oc1ccc(C#Cc3cnn(CCC#Cc4ccc(OCc5ccc(F)c(C(F)(F)F)c5)c(CNCCO)c4)c3)cc1O2)c1ccc(Cl)cc1] |
| 164 | 877.42 | 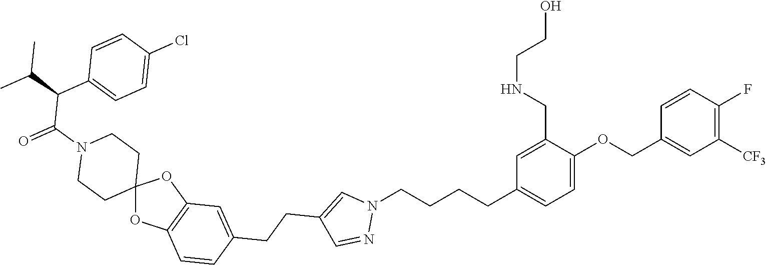 [SMILES: CC(C)[C@H](C(=O)N1CCC2(CC1)Oc1ccc(CCc3cnn(CCCCc4ccc(OCc5ccc(F)c(C(F)(F)F)c5)c(CNCCO)c4)c3)cc1O2)c1ccc(Cl)cc1] |
| 165 | 913.41 | 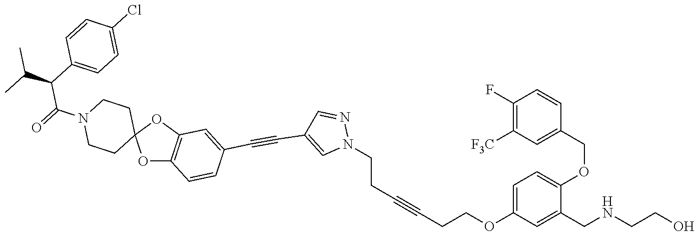 [SMILES: CC(C)[C@H](C(=O)N1CCC2(CC1)Oc1ccc(C#Cc3cnn(CCC#CCCOc4ccc(OCc5ccc(F)c(C(F)(F)F)c5)c(CNCCO)c4)c3)cc1O2)c1ccc(Cl)cc1] |
| 166 | 921.47 | 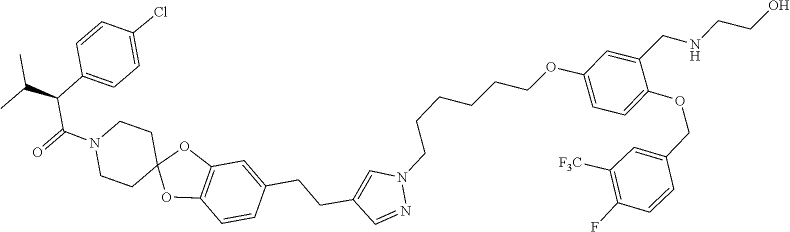 [SMILES: CC(C)[C@H](C(=O)N1CCC2(CC1)Oc1ccc(CCc3cnn(CCCCCCOc4ccc(OCc5ccc(F)c(C(F)(F)F)c5)c(CNCCO)c4)c3)cc1O2)c1ccc(Cl)cc1] |
| 167 | 916.15 | 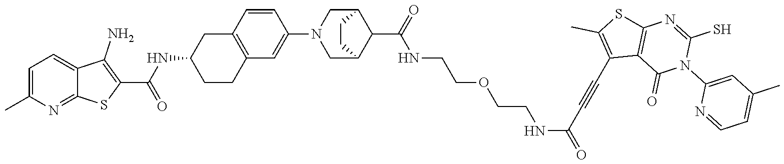 |
| 168 | 970.24 | 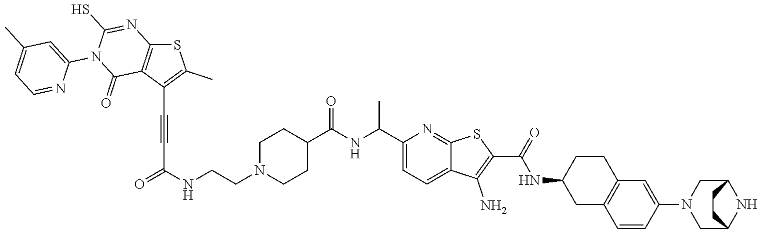 [SMILES: Cc1ccnc(-n2c(S)nc3sc(C)c(C#CC(=O)NCCN4CCC(C(=O)NC(C)c5ccc6c(N)c(C(=O)N[C@H]7CCc8cc(N9C[C@H]%10CC[C@@H](C9)N%10)ccc8C7)sc6n5)CC4)c3c2=O)c1] |
| 169 | 859.04 | 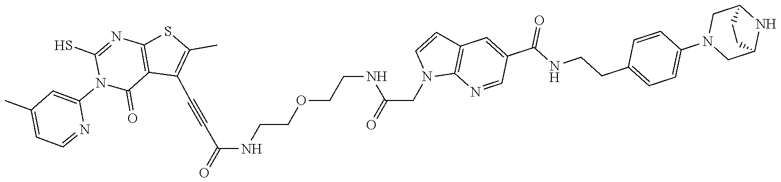 [SMILES: Cc1ccnc(-n2c(S)nc3sc(C)c(C#CC(=O)NCCOCCNC(=O)Cn4ccc5cc(C(=O)NCCc6ccc(N7C[C@H]8CC[C@@H](C7)N8)cc6)cnc54)c3c2=O)c1] |
| 170 | 785.99 | 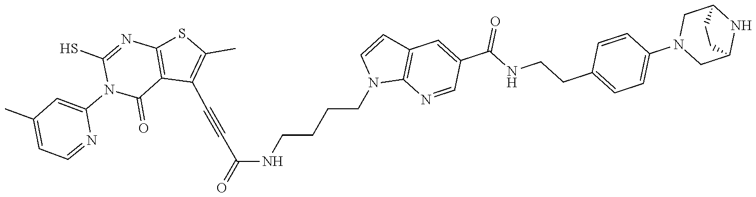 [SMILES: Cc1ccnc(-n2c(S)nc3sc(C)c(C#CC(=O)NCCCCn4ccc5cc(C(=O)NCCc6ccc(N7C[C@H]8CC[C@@H](C7)N8)cc6)cnc54)c3c2=O)c1] |
| 171 | 858.11 | 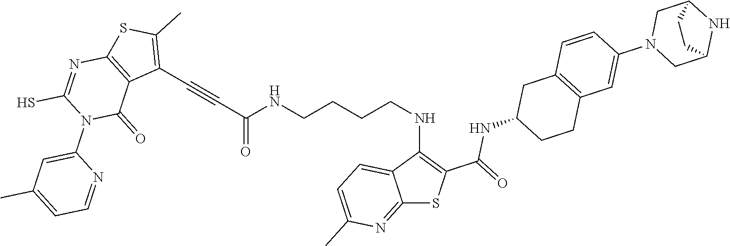 [SMILES: Cc1ccnc(-n2c(S)nc3sc(C)c(C#CC(=O)NCCCCNc4c(C(=O)N[C@H]5CCc6cc(N7C[C@H]8CC[C@@H](C7)N8)ccc6C5)sc5nc(C)ccc45)c3c2=O)c1] |
| 172 | 837.01 | 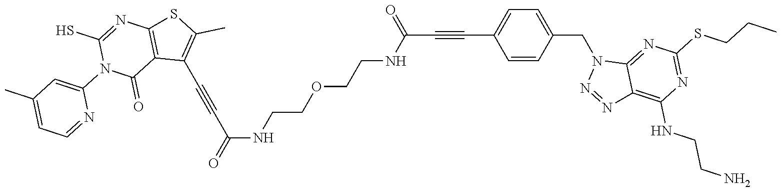 [SMILES: CCCSc1nc(NCCN)c2nnn(Cc3ccc(C#CC(=O)NCCOCCNC(=O)C#Cc4c(C)sc5nc(S)n(-c6cc(C)ccn6)c(=O)c45)cc3)c2n1] |
| 173 | 761.34 | 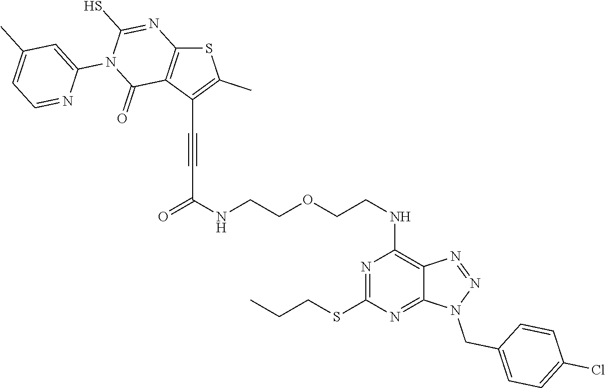 [SMILES: CCCSc1nc(NCCOCCNC(=O)C#Cc2c(C)sc3nc(S)n(-c4cc(C)ccn4)c(=O)c23)c2nnn(Cc3ccc(Cl)cc3)c2n1] |
| 174 | 837.01 | 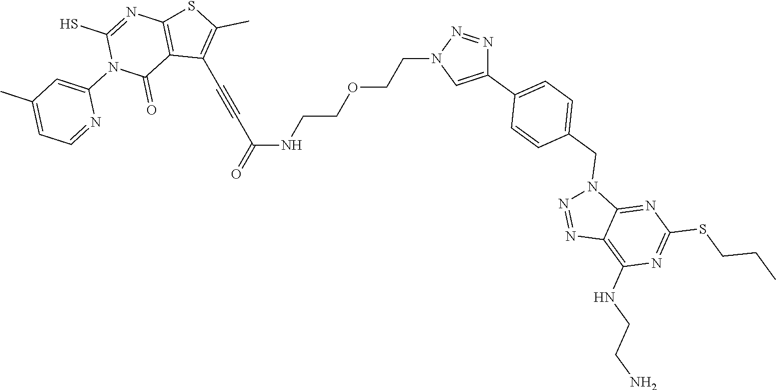 [SMILES: CCCSc1nc(NCCN)c2nnn(Cc3ccc(-c4cn(CCOCCNC(=O)C#Cc5c(C)sc6nc(S)n(-c7cc(C)ccn7)c(=O)c56)nn4)cc3)c2n1] |
| 175 | 843.40 | 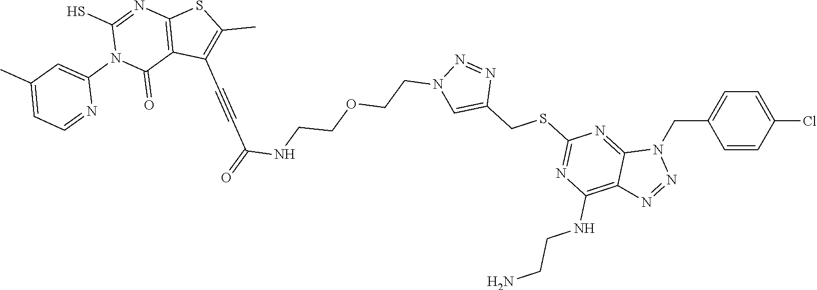 [SMILES: Cc1ccnc(-n2c(S)nc3sc(C)c(C#CC(=O)NCCOCCn4cc(CSc5nc(NCCN)c6nnn(Cc7ccc(Cl)cc7)c6n5)nn4)c3c2=O)c1] |
| 176 | 884.93 | 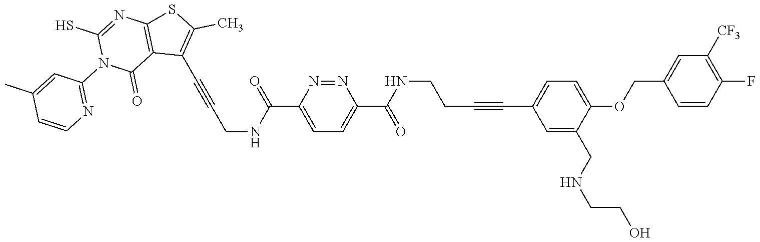 [SMILES: Cc1ccnc(-n2c(S)nc3sc(C)c(C#CCNC(=O)c4ccc(C(=O)NCCC#Cc5ccc(OCc6ccc(F)c(C(F)(F)F)c6)c(CNCCO)c5)nn4)c3c2=O)c1] |
| 177 | 892.99 | 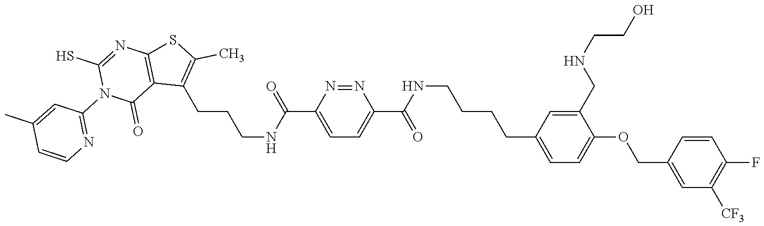 [SMILES: Cc1ccnc(-n2c(S)nc3sc(C)c(CCCNC(=O)c4ccc(C(=O)NCCCCc5ccc(OCc6ccc(F)c(C(F)(F)F)c6)c(CNCCO)c5)nn4)c3c2=O)c1] |
| 178 | 875.80 | 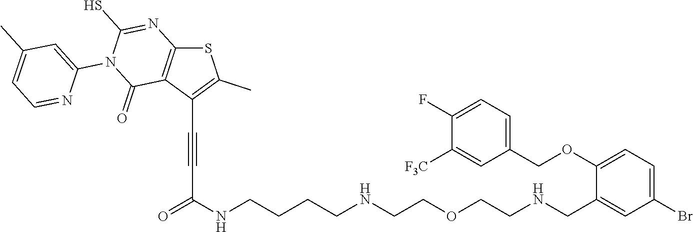 [SMILES: Cc1ccnc(-n2c(S)nc3sc(C)c(C#CC(=O)NCCCCNCCOCCNCc4cc(Br)ccc4OCc4ccc(F)c(C(F)(F)F)c4)c3c2=O)c1] |
| 179 | 879.83 | 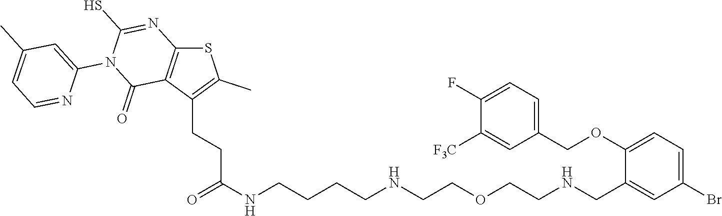 [SMILES: Cc1ccnc(-n2c(S)nc3sc(C)c(CCC(=O)NCCCCNCCOCCNCc4cc(Br)ccc4OCc4ccc(F)c(C(F)(F)F)c4)c3c2=O)c1] |
| 180 | 1004.18 | 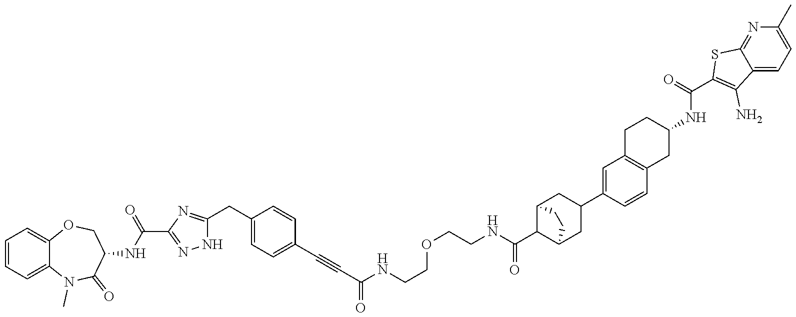 [SMILES: Cc1ccc2c(N)c(C(=O)N[C@H]3CCc4cc(C5C[C@H]6CC[C@@H](C5)C6C(=O)NCCOCCNC(=O)C#Cc5ccc(Cc6nc(C(=O)N[C@H]7COc8ccccc8N(C)C7=O)n[nH]6)cc5)ccc4C3)sc2n1] |
| 181 | 1058.28 | 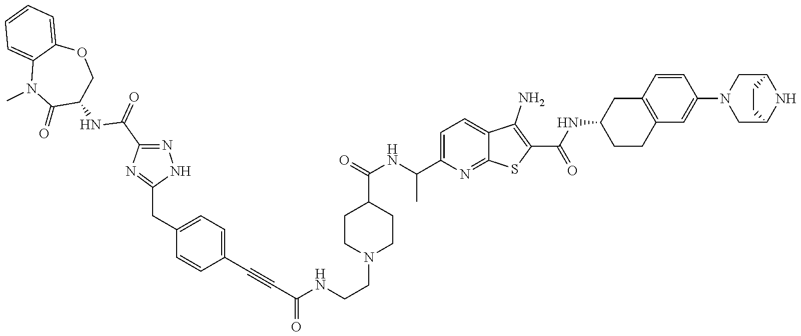 [SMILES: CC(NC(=O)C1CCN(CCNC(=O)C#Cc2ccc(Cc3nc(C(=O)N[C@H]4COc5ccccc5N(C)C4=O)n[nH]3)cc2)CC1)c1ccc2c(N)c(C(=O)N[C@H]3CCc4cc(N5C[C@H]6CC[C@@H](C5)N6)ccc4C3)sc2n1] |
| 182 | 947.07 | 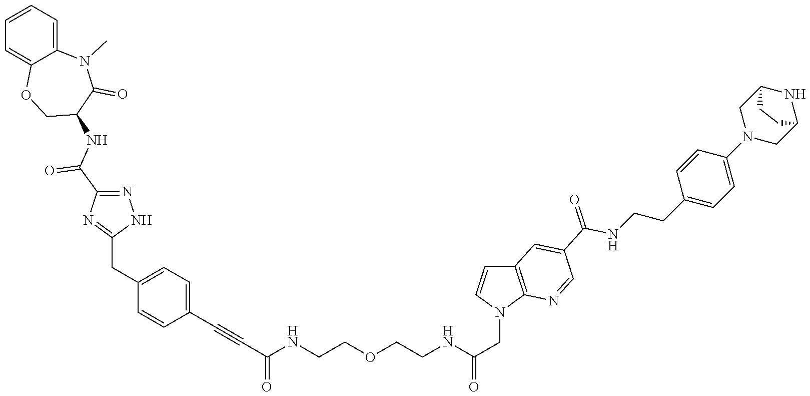 [SMILES: CN1C(=O)[C@@H](NC(=O)c2n[nH]c(Cc3ccc(C#CC(=O)NCCOCCNC(=O)Cn4ccc5cc(C(=O)NCCc6ccc(N7C[C@H]8CC[C@@H](C7)N8)cc6)cnc54)cc3)n2)COc2ccccc21] |
| 183 | 874.02 | 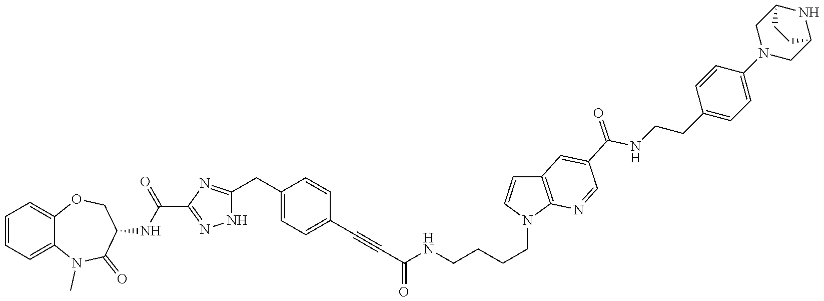 [SMILES: CN1C(=O)[C@@H](NC(=O)c2n[nH]c(Cc3ccc(C#CC(=O)NCCCCn4ccc5cc(C(=O)NCCc6ccc(N7C[C@H]8CC[C@@H](C7)N8)cc6)cnc54)cc3)n2)COc2ccccc21] |
| 184 | 946.14 | 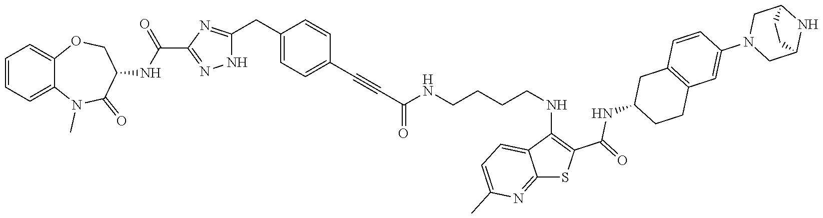 [SMILES: Cc1ccc2c(NCCCCNC(=O)C#Cc3ccc(Cc4nc(C(=O)N[C@H]5COc6ccccc6N(C)C5=O)n[nH]4)cc3)c(C(=O)N[C@H]3CCc4cc(N5C[C@H]6CC[C@@H](C5)N6)ccc4C3)sc2n1] |
| 185 | 925.04 | 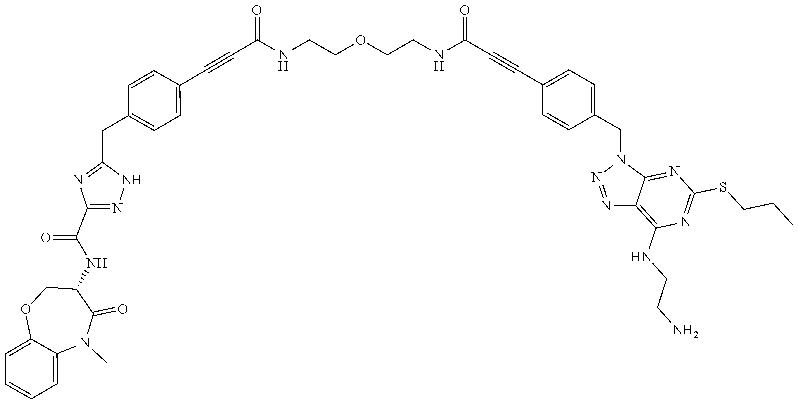 [SMILES: CCCSc1nc(NCCN)c2nnn(Cc3ccc(C#CC(=O)NCCOCCNC(=O)C#Cc4ccc(Cc5nc(C(=O)N[C@H]6COc7ccccc7N(C)C6=O)n[nH]5)cc4)cc3)c2n1] |
| 186 | 849.37 | 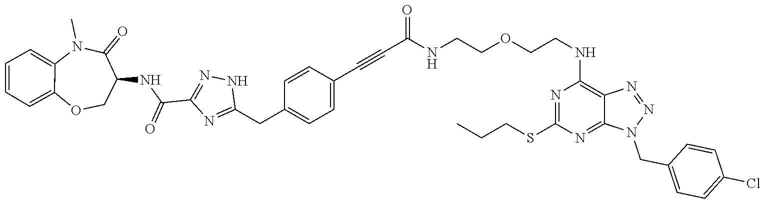 [SMILES: CCCSc1nc(NCCOCCNC(=O)C#Cc2ccc(Cc3nc(C(=O)N[C@H]4COc5ccccc5N(C)C4=O)n[nH]3)cc2)c2nnn(Cc3ccc(Cl)cc3)c2n1] |
| 187 | 925.05 | 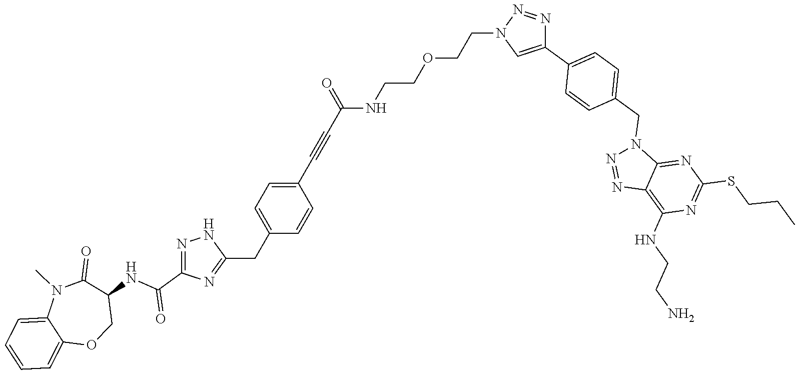 [SMILES: CCCSc1nc(NCCN)c2nnn(Cc3ccc(-c4cn(CCOCCNC(=O)C#Cc5ccc(Cc6nc(C(=O)N[C@H]7COc8ccccc8N(C)C7=O)n[nH]6)cc5)nn4)cc3)c2n1] |
| 188 | 931.43 | 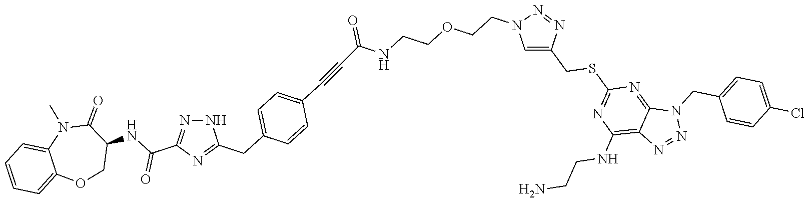 [SMILES: CN1C(=O)[C@@H](NC(=O)c2n[nH]c(Cc3ccc(C#CC(=O)NCCOCCn4cc(CSc5nc(NCCN)c6nnn(Cc7ccc(Cl)cc7)c6n5)nn4)cc3)n2)COc2ccccc21] |
| 189 | 955.97 | 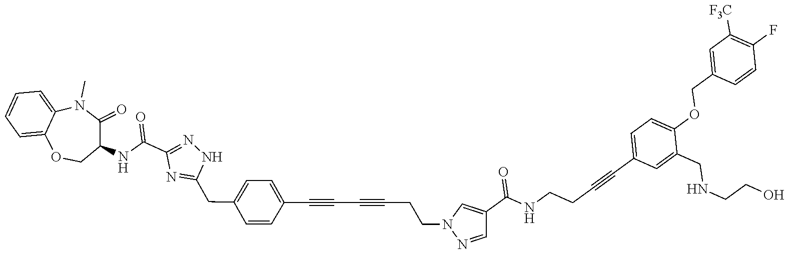 [SMILES: CN1C(=O)[C@@H](NC(=O)c2n[nH]c(Cc3ccc(C#CC#CCCn4cc(C(=O)NCCC#Cc5ccc(OCc6ccc(F)c(C(F)(F)F)c6)c(CNCCO)c5)cn4)cc3)n2)COc2ccccc21] |
| 190 | 968.07 | 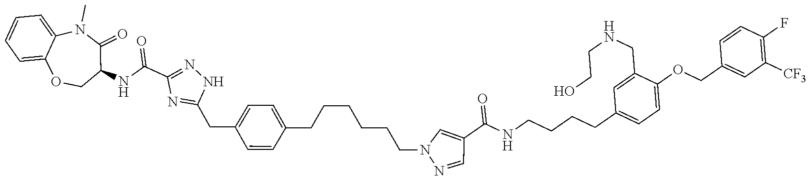 [SMILES: CN1C(=O)[C@@H](NC(=O)c2n[nH]c(Cc3ccc(CCCCCCn4cc(C(=O)NCCCCc5ccc(OCc6ccc(F)c(C(F)(F)F)c6)c(CNCCO)c5)cn4)cc3)n2)COc2ccccc21] |
| 191 | 982.07 | 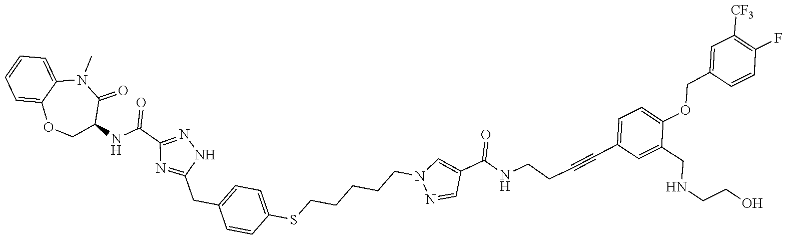 [SMILES: CN1C(=O)[C@@H](NC(=O)c2n[nH]c(Cc3ccc(SCCCCCn4cc(C(=O)NCCC#Cc5ccc(OCc6ccc(F)c(C(F)(F)F)c6)c(CNCCO)c5)cn4)cc3)n2)COc2ccccc21] |
| 192 | 986.10 | 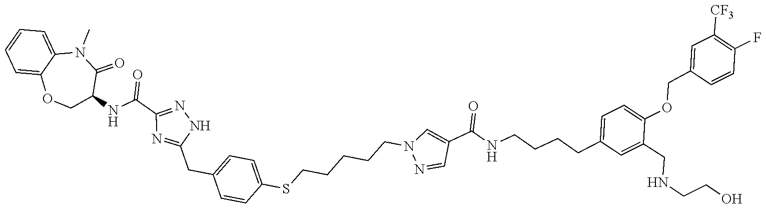 [SMILES: CN1C(=O)[C@@H](NC(=O)c2n[nH]c(Cc3ccc(SCCCCCn4cc(C(=O)NCCCCc5ccc(OCc6ccc(F)c(C(F)(F)F)c6)c(CNCCO)c5)cn4)cc3)n2)COc2ccccc21] |
| 193 | 916.13 | 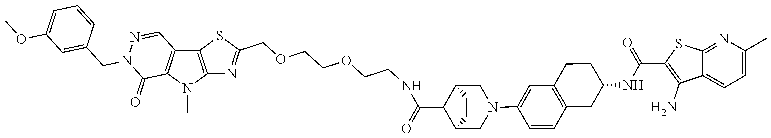 |
| 194 | 970.23 | 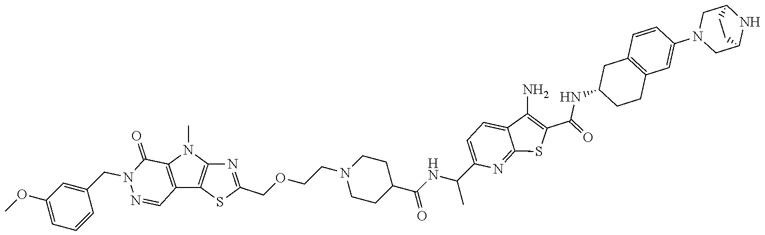 [SMILES: COc1cccc(Cn2ncc3c4sc(COCCN5CCC(C(=O)NC(C)c6ccc7c(N)c(C(=O)N[C@H]8CCc9cc(N%10C[C@H]%11CC[C@@H](C%10)N%11)ccc9C8)sc7n6)CC5)nc4n(C)c3c2=O)c1] |
| 195 | 859.02 | 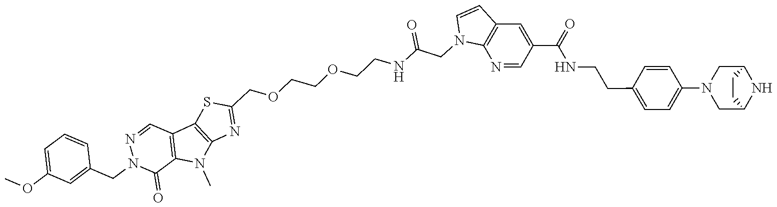 [SMILES: COc1cccc(Cn2ncc3c4sc(COCCOCCNC(=O)Cn5ccc6cc(C(=O)NCCc7ccc(N8C[C@H]9CC[C@@H](C8)N9)cc7)cnc65)nc4n(C)c3c2=O)c1] |
| 196 | 785.97 | 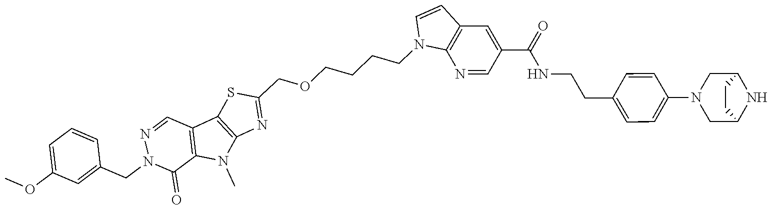 [SMILES: COc1cccc(Cn2ncc3c4sc(COCCCCn5ccc6cc(C(=O)NCCc7ccc(N8C[C@H]9CC[C@@H](C8)N9)cc7)cnc65)nc4n(C)c3c2=O)c1] |
| 197 | 942.22 | 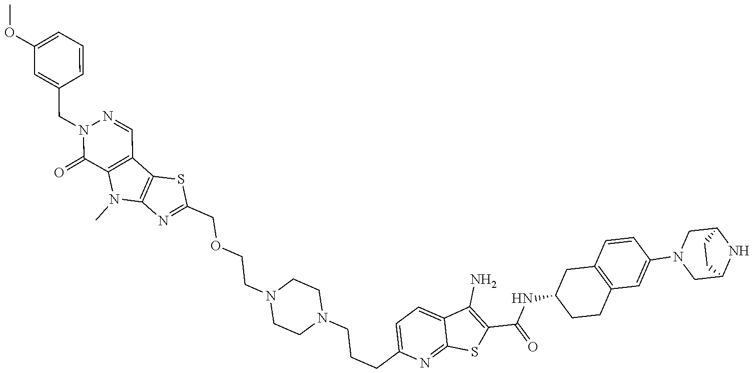 [SMILES: COc1cccc(Cn2ncc3c4sc(COCCN5CCN(CCCc6ccc7c(N)c(C(=O)N[C@H]8CCc9cc(N%10C[C@H]%11CC[C@@H](C%10)N%11)ccc9C8)sc7n6)CC5)nc4n(C)c3c2=O)c1] |
| 198 | 858.09 | 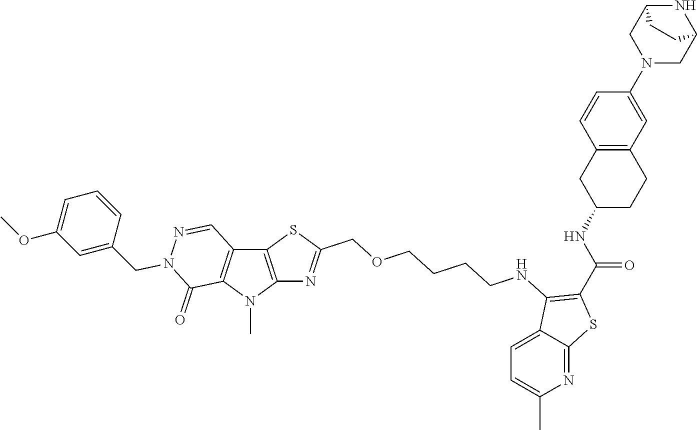 [SMILES: COc1cccc(Cn2ncc3c4sc(COCCCCNc5c(C(=O)N[C@H]6CCc7cc(N8C[C@H]9CC[C@@H](C8)N9)ccc7C6)sc6nc(C)ccc56)nc4n(C)c3c2=O)c1] |
| 199 | 836.99 | 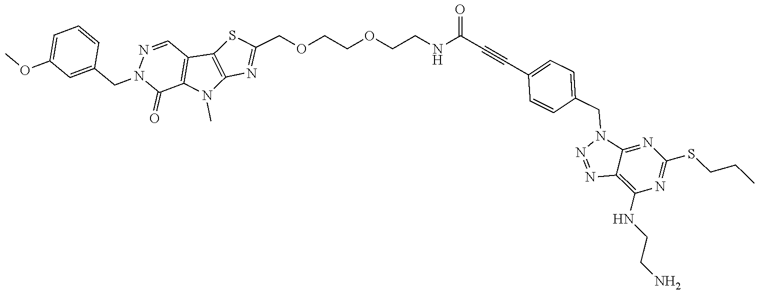 [SMILES: CCCSc1nc(NCCN)c2nnn(Cc3ccc(C#CC(=O)NCCOCCOCc4nc5c(s4)c4cnn(Cc6cccc(OC)c6)c(=O)c4n5C)cc3)c2n1] |
| 200 | 761.32 | 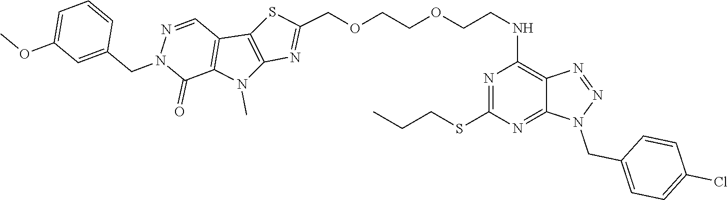 [SMILES: CCCSc1nc(NCCOCCOCc2nc3c(s2)c2cnn(Cc4cccc(OC)c4)c(=O)c2n3C)c2nnn(Cc3ccc(Cl)cc3)c2n1] |
| 201 | 778.31 | 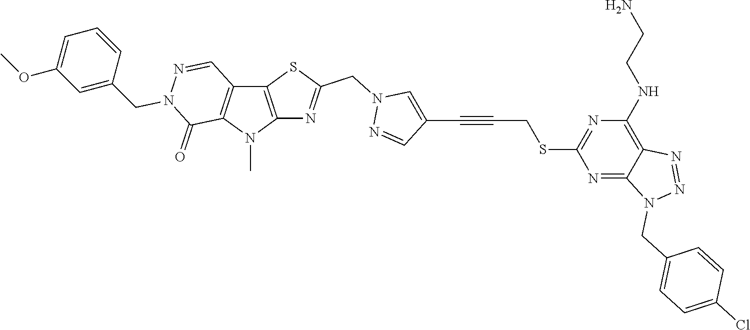 [SMILES: COc1cccc(Cn2ncc3c4sc(Cn5cc(C#CCSc6nc(NCCN)c7nnn(Cc8ccc(Cl)cc8)c7n6)cn5)nc4n(C)c3c2=O)c1] |
| 202 | 782.34 | 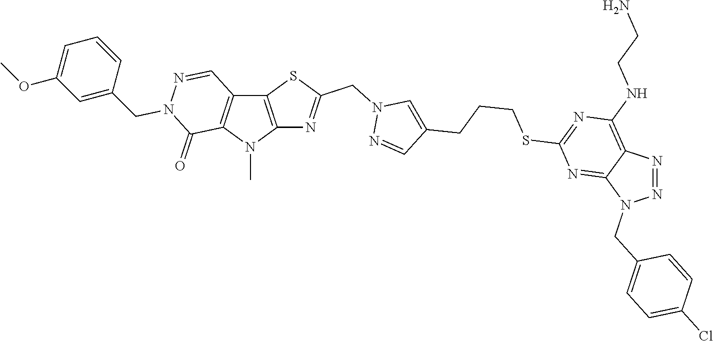 [SMILES: COc1cccc(Cn2ncc3c4sc(Cn5cc(CCCSc6nc(NCCN)c7nnn(Cc8ccc(Cl)cc8)c7n6)cn5)nc4n(C)c3c2=O)c1] |
| 203 | 903.99 | 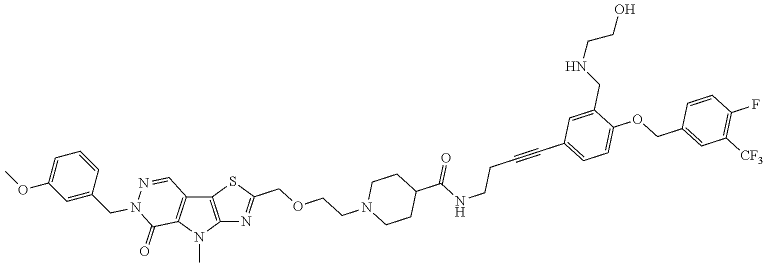 [SMILES: COc1cccc(Cn2ncc3c4sc(COCCN5CCC(C(=O)NCCC#Cc6ccc(OCc7ccc(F)c(C(F)(F)F)c7)c(CNCCO)c6)CC5)nc4n(C)c3c2=O)c1] |
| 204 | 908.03 | 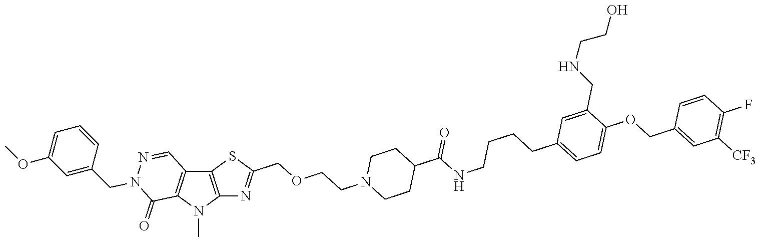 [SMILES: COc1cccc(Cn2ncc3c4sc(COCCN5CCC(C(=O)NCCCCc6ccc(OCc7ccc(F)c(C(F)(F)F)c7)c(CNCCO)c6)CC5)nc4n(C)c3c2=O)c1] |
| 205 | 815.84 | 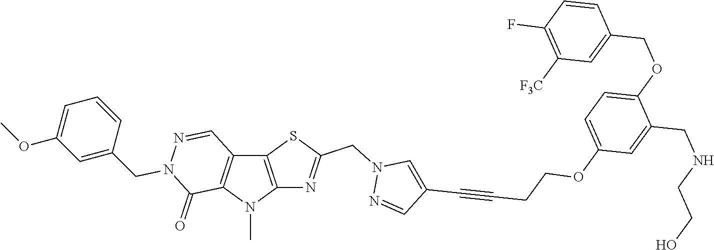 [SMILES: COc1cccc(Cn2ncc3c4sc(Cn5cc(C#CCCOc6ccc(OCc7ccc(F)c(C(F)(F)F)c7)c(CNCCO)c6)cn5)nc4n(C)c3c2=O)c1] |
| 206 | 819.88 | 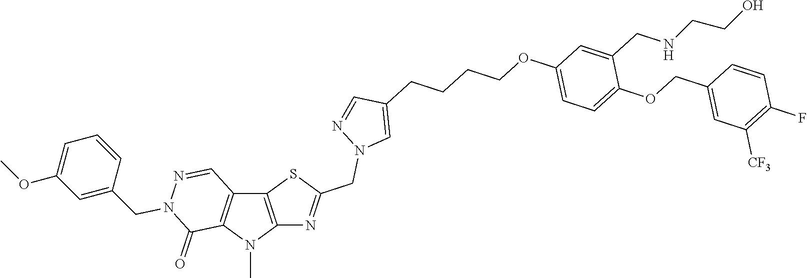 [SMILES: COc1cccc(Cn2ncc3c4sc(Cn5cc(CCCCOc6ccc(OCc7ccc(F)c(C(F)(F)F)c7)c(CNCCO)c6)cn5)nc4n(C)c3c2=O)c1] |
| 207 | 850.89 | 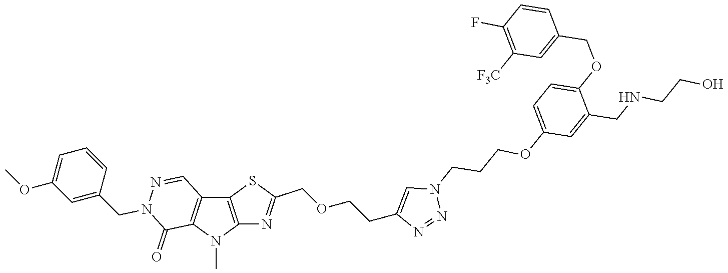 [SMILES: COc1cccc(Cn2ncc3c4sc(COCCc5cn(CCCOc6ccc(OCc7ccc(F)c(C(F)(F)F)c7)c(CNCCO)c6)nn5)nc4n(C)c3c2=O)c1] |
| 208 | 889.93 | 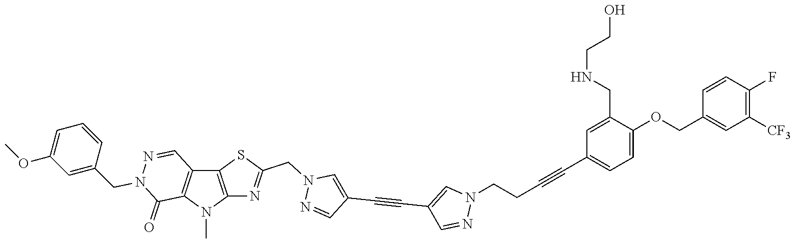 [SMILES: COc1cccc(Cn2ncc3c4sc(Cn5cc(C#Cc6cnn(CCC#Cc7ccc(OCc8ccc(F)c(C(F)(F)F)c8)c(CNCCO)c7)c6)cn5)nc4n(C)c3c2=O)c1] |
| 209 | 897.99 | 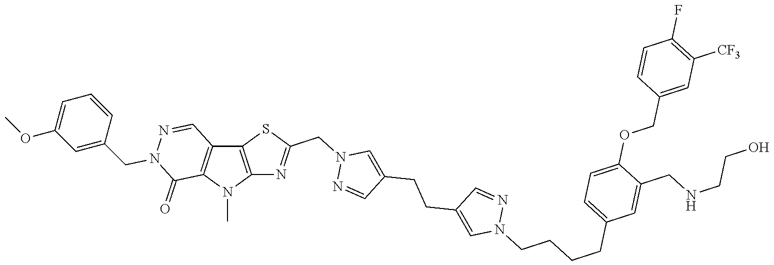 [SMILES: COc1cccc(Cn2ncc3c4sc(Cn5cc(CCc6cnn(CCCCc7ccc(OCc8ccc(F)c(C(F)(F)F)c8)c(CNCCO)c7)c6)cn5)nc4n(C)c3c2=O)c1] |
| 210 | 822.84 | 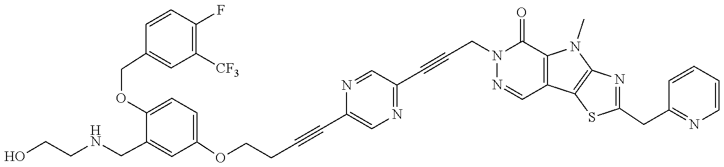 [SMILES: Cn1c2nc(Cc3ccccn3)sc2c2cnn(CC#Cc3cnc(C#CCCOc4ccc(OCc5ccc(F)c(C(F)(F)F)c5)c(CNCCO)c4)cn3)c(=O)c21] |
| 211 | 830.90 | 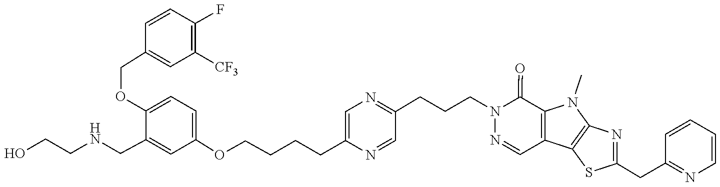 [SMILES: Cn1c2nc(Cc3ccccn3)sc2c2cnn(CCCc3cnc(CCCCOc4ccc(OCc5ccc(F)c(C(F)(F)F)c5)c(CNCCO)c4)cn3)c(=O)c21] |
| 212 | 822.84 | 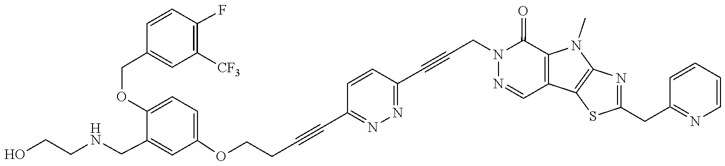 [SMILES: Cn1c2nc(Cc3ccccn3)sc2c2cnn(CC#Cc3ccc(C#CCCOc4ccc(OCc5ccc(F)c(C(F)(F)F)c5)c(CNCCO)c4)nn3)c(=O)c21] |
| 213 | 830.90 | 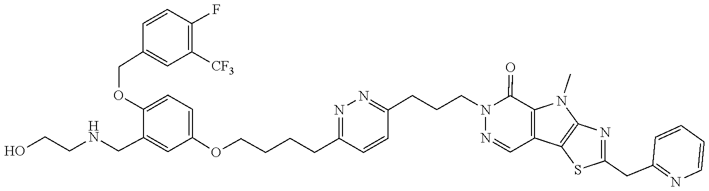 [SMILES: Cn1c2nc(Cc3ccccn3)sc2c2cnn(CCCc3ccc(CCCCOc4ccc(OCc5ccc(F)c(C(F)(F)F)c5)c(CNCCO)c4)nn3)c(=O)c21] |
| 214 | 744.77 | 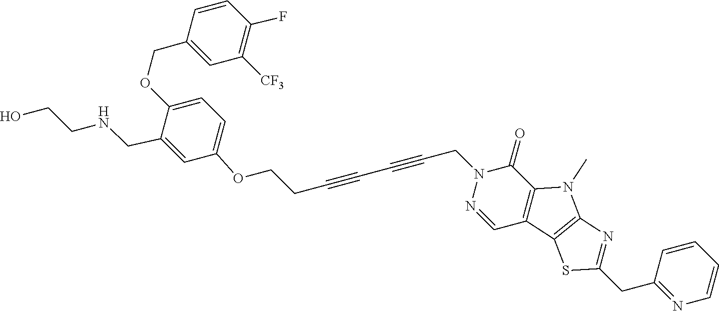 [SMILES: Cn1c2nc(Cc3ccccn3)sc2c2cnn(CC#CC#CCCOc3ccc(OCc4ccc(F)c(C(F)(F)F)c4)c(CNCCO)c3)c(=O)c21] |
| 215 | 752.83 | 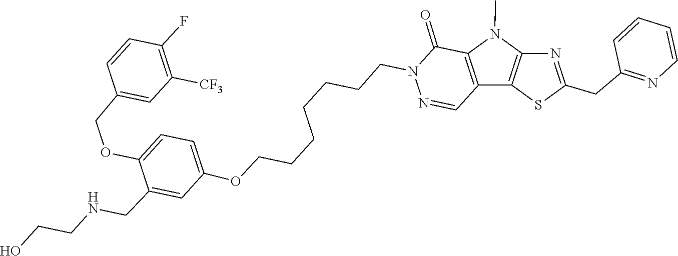 [SMILES: Cn1c2nc(Cc3ccccn3)sc2c2cnn(CCCCCCCOc3ccc(OCc4ccc(F)c(C(F)(F)F)c4)c(CNCCO)c3)c(=O)c21] |
| 216 | 1099.67 | 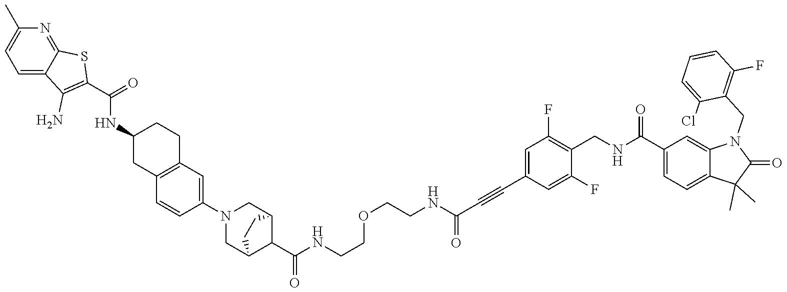 [SMILES: Cc1ccc2c(N)c(C(=O)N[C@H]3CCc4cc(N5C[C@H]6CC[C@@H](C5)C6C(=O)NCCOCCNC(=O)C#Cc5cc(F)c(CNC(=O)c6ccc7c(c6)N(Cc6c(F)cccc6Cl)C(=O)C7(C)C)c(F)c5)ccc4C3)sc2n1] |
| 217 | 1434.78 | 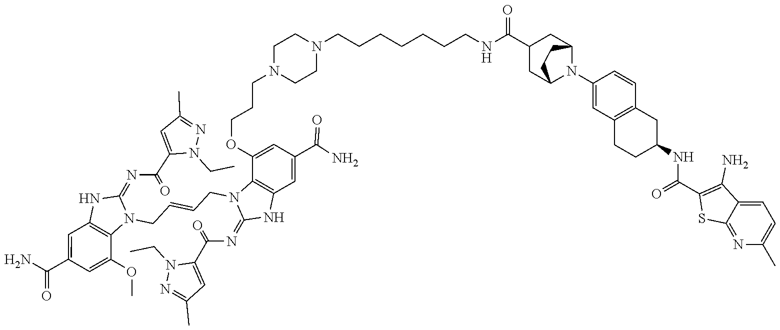 [SMILES: CCn1nc(C)cc1C(=O)/N=c1/[nH]c2cc(C(N)=O)cc(OC)c2n1C/C=C/Cn1/c(=N\C(=O)c2cc(C)nn2CC)[nH]c2cc(C(N)=O)cc(OCCCN3CCN(CCCCCCCNC(=O)C4C[C@H]5CC[C@@H](C4)N5c4ccc5c(c4)CC[C@H](NC(=O)c4sc6nc(C)ccc6c4N)C5)CC3)c21] |
| 218 | 1153.76 | 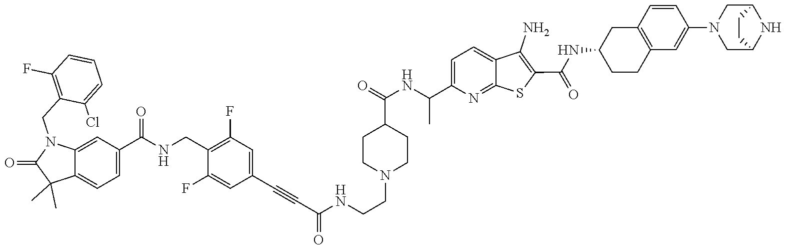 [SMILES: CC(NC(=O)C1CCN(CCNC(=O)C#Cc2cc(F)c(CNC(=O)c3ccc4c(c3)N(Cc3c(F)cccc3Cl)C(=O)C4(C)C)c(F)c2)CC1)c1ccc2c(N)c(C(=O)N[C@H]3CCc4cc(N5C[C@H]6CC[C@@H](C5)N6)ccc4C3)sc2n1] |
| 219 | 1449.80 | 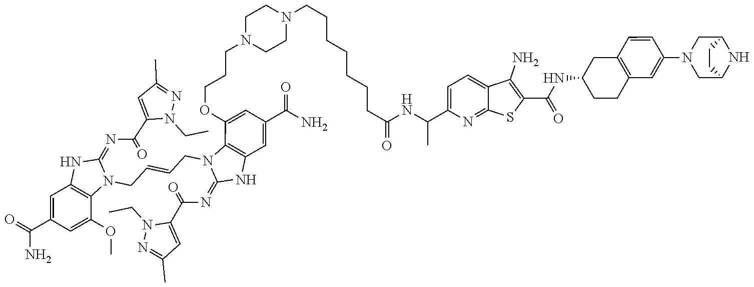 [SMILES: CCn1nc(C)cc1C(=O)/N=c1/[nH]c2cc(C(N)=O)cc(OC)c2n1C/C=C/Cn1/c(=N\C(=O)c2cc(C)nn2CC)[nH]c2cc(C(N)=O)cc(OCCCN3CCN(CCCCCCCC(=O)NC(C)c4ccc5c(N)c(C(=O)N[C@H]6CCc7cc(N8C[C@H]9CC[C@@H](C8)N9)ccc7C6)sc5n4)CC3)c21] |
| 220 | 1042.56 | 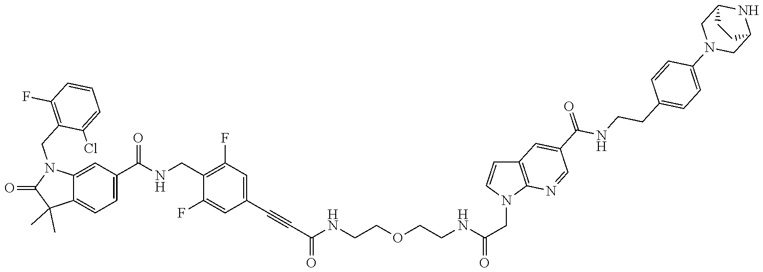 [SMILES: CC1(C)C(=O)N(Cc2c(F)cccc2Cl)c2cc(C(=O)NCc3c(F)cc(C#CC(=O)NCCOCCNC(=O)Cn4ccc5cc(C(=O)NCCc6ccc(N7C[C@H]8CC[C@@H](C7)N8)cc6)cnc54)cc3F)ccc21] |
| 221 | 1377.67 | 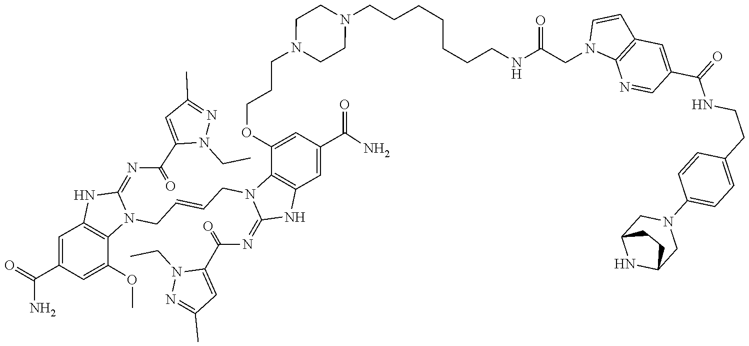 [SMILES: CCn1nc(C)cc1C(=O)/N=c1/[nH]c2cc(C(N)=O)cc(OC)c2n1C/C=C/Cn1/c(=N\C(=O)c2cc(C)nn2CC)[nH]c2cc(C(N)=O)cc(OCCCN3CCN(CCCCCCCNC(=O)Cn4ccc5cc(C(=O)NCCc6ccc(N7C[C@H]8CC[C@@H](C7)N8)cc6)cnc54)CC3)c21] |
| 222 | 969.51 | 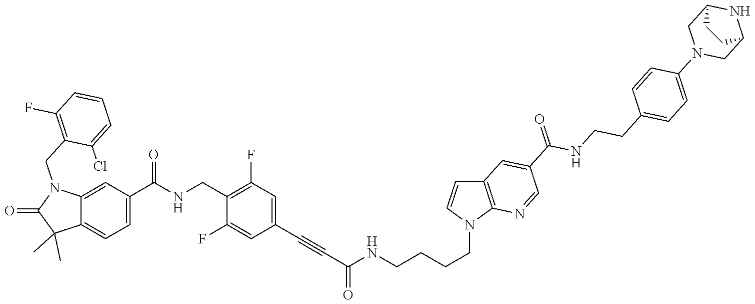 [SMILES: CC1(C)C(=O)N(Cc2c(F)cccc2Cl)c2cc(C(=O)NCc3c(F)cc(C#CC(=O)NCCCCn4ccc5cc(C(=O)NCCc6ccc(N7C[C@H]8CC[C@@H](C7)N8)cc6)cnc54)cc3F)ccc21] |
| 223 | 1306.59 | 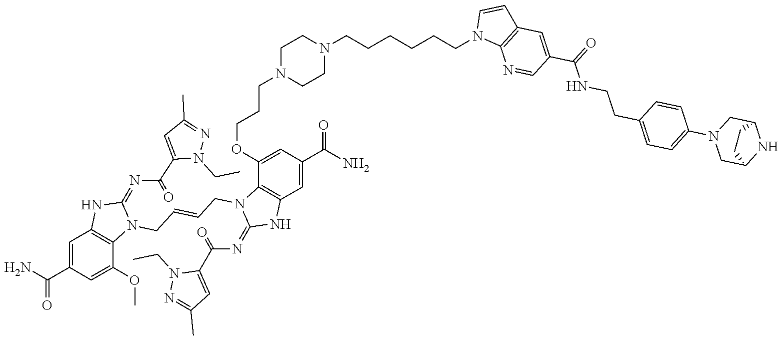 [SMILES: CCn1nc(C)cc1C(=O)/N=c1/[nH]c2cc(C(N)=O)cc(OC)c2n1C/C=C/Cn1/c(=N\C(=O)c2cc(C)nn2CC)[nH]c2cc(C(N)=O)cc(OCCCN3CCN(CCCCCCn4ccc5cc(C(=O)NCCc6ccc(N7C[C@H]8CC[C@@H](C7)N8)cc6)cnc54)CC3)c21] |
| 224 | 1408.79 | 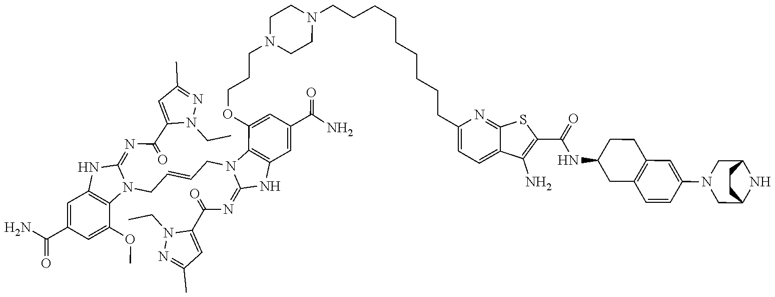 [SMILES: CCn1nc(C)cc1C(=O)/N=c1/[nH]c2cc(C(N)=O)cc(OC)c2n1C/C=C/Cn1/c(=N\C(=O)c2cc(C)nn2CC)[nH]c2cc(C(N)=O)cc(OCCCN3CCN(CCCCCCCCCc4ccc5c(N)c(C(=O)N[C@H]6CCc7cc(N8C[C@H]9CC[C@@H](C8)N9)ccc7C6)sc5n4)CC3)c21] |
| 225 | 1041.63 | 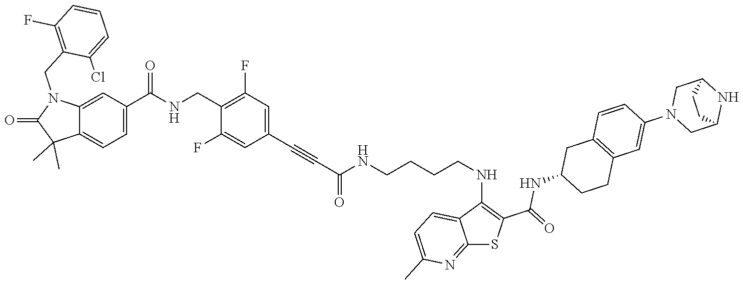 [SMILES: Cc1ccc2c(NCCCCNC(=O)C#Cc3cc(F)c(CNC(=O)c4ccc5c(c4)N(Cc4c(F)cccc4Cl)C(=O)C5(C)C)c(F)c3)c(C(=O)N[C@H]3CCc4cc(N5C[C@H]6CC[C@@H](C5)N6)ccc4C3)sc2n1] |
| 226 | 1378.72 | 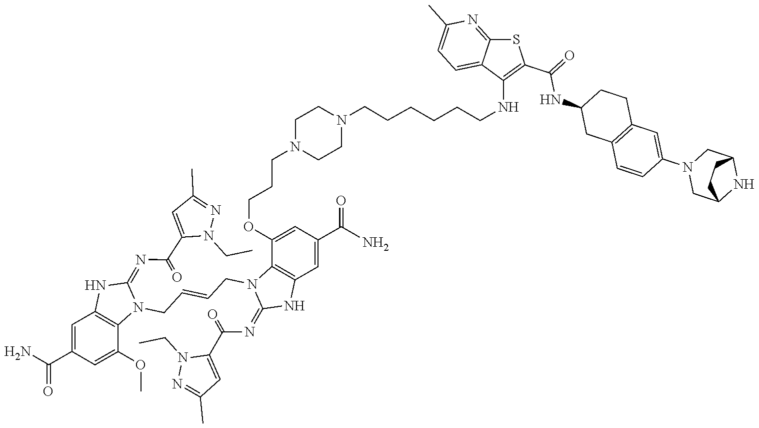 [SMILES: CCn1nc(C)cc1C(=O)/N=c1/[nH]c2cc(C(N)=O)cc(OC)c2n1C/C=C/Cn1/c(=N\C(=O)c2cc(C)nn2CC)[nH]c2cc(C(N)=O)cc(OCCCN3CCN(CCCCCCNc4c(C(=O)N[C@H]5CCc6cc(N7C[C@H]8CC[C@@H](C7)N8)ccc6C5)sc5nc(C)ccc45)CC3)c21] |
| 227 | 1020.53 | 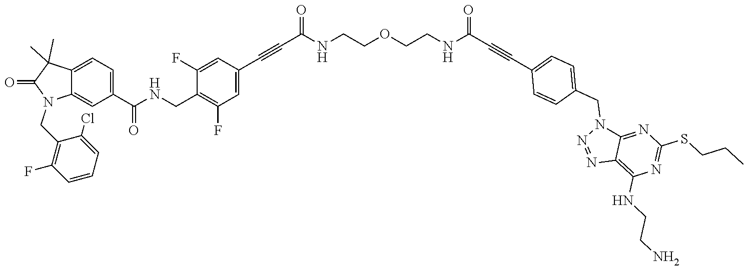 [SMILES: CCCSc1nc(NCCN)c2nnn(Cc3ccc(C#CC(=O)NCCOCCNC(=O)C#Cc4cc(F)c(CNC(=O)c5ccc6c(c5)N(Cc5c(F)cccc5Cl)C(=O)C6(C)C)c(F)c4)cc3)c2n1] |
| 228 | 944.86 | 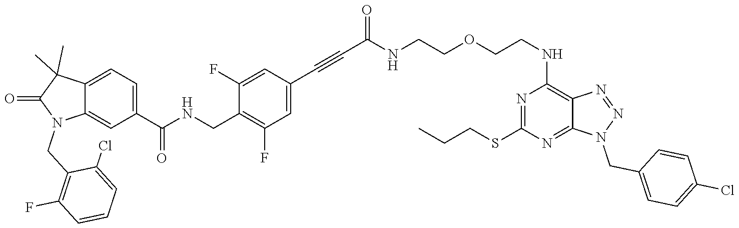 [SMILES: CCCSc1nc(NCCOCCNC(=O)C#Cc2cc(F)c(CNC(=O)c3ccc4c(c3)N(Cc3c(F)cccc3Cl)C(=O)C4(C)C)c(F)c2)c2nnn(Cc3ccc(Cl)cc3)c2n1] |
| 229 | 1355.64 | 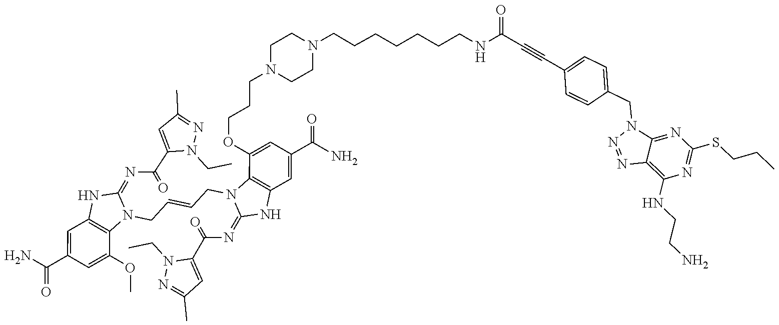 [SMILES: CCCSc1nc(NCCN)c2nnn(Cc3ccc(C#CC(=O)NCCCCCCCN4CCN(CCCOc5cc(C(N)=O)cc6[nH]/c(=N/C(=O)c7cc(C)nn7CC)n(C/C=C/Cn7/c(=N\C(=O)c8cc(C)nn8CC)[nH]c8cc(C(N)=O)cc(OC)c87)c56)CC4)cc3)c2n1] |
| 230 | 1265.94 | 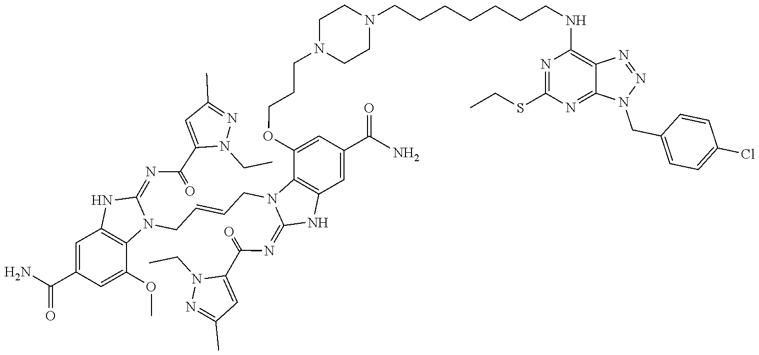 [SMILES: CCSc1nc(NCCCCCCCN2CCN(CCCOc3cc(C(N)=O)cc4[nH]/c(=N/C(=O)c5cc(C)nn5CC)n(C/C=C/Cn5/c(=N\C(=O)c6cc(C)nn6CC)[nH]c6cc(C(N)=O)cc(OC)c65)c34)CC2)c2nnn(Cc3ccc(Cl)cc3)c2n1] |
| 231 | 1144.59 | 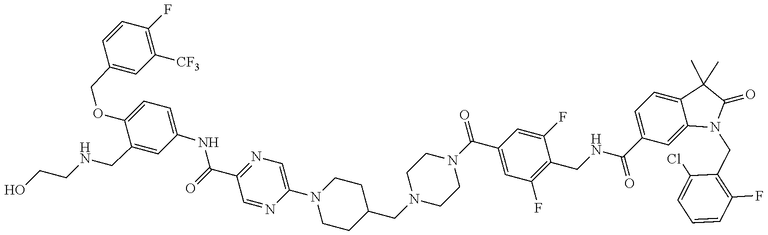 [SMILES: CC1(C)C(=O)N(Cc2c(F)cccc2Cl)c2cc(C(=O)NCc3c(F)cc(C(=O)N4CCN(CC5CCN(c6cnc(C(=O)Nc7ccc(OCc8ccc(F)c(C(F)(F)F)c8)c(CNCCO)c7)cn6)CC5)CC4)cc3F)ccc21] |
| 232 | 956.36 | 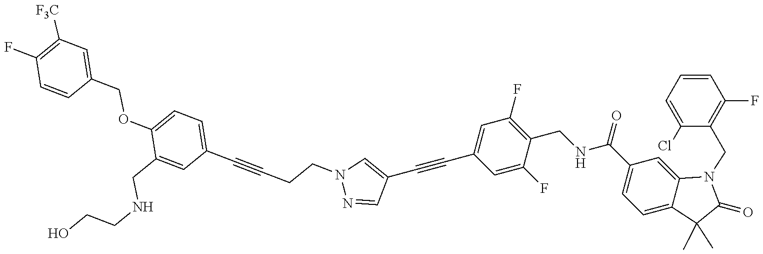 [SMILES: CC1(C)C(=O)N(Cc2c(F)cccc2Cl)c2cc(C(=O)NCc3c(F)cc(C#Cc4cnn(CCC#Cc5ccc(OCc6ccc(F)c(C(F)(F)F)c6)c(CNCCO)c5)c4)cc3F)ccc21] |
| 233 | 964.42 | 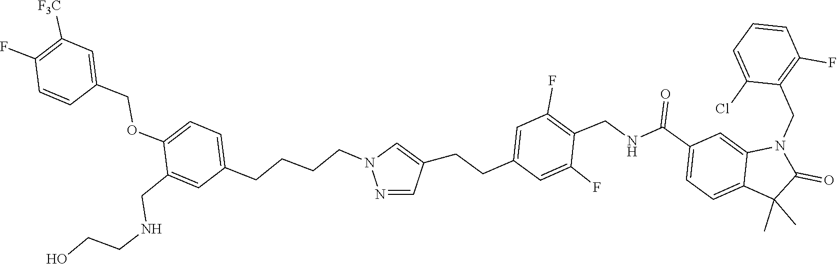 [SMILES: CC1(C)C(=O)N(Cc2c(F)cccc2Cl)c2cc(C(=O)NCc3c(F)cc(CCc4cnn(CCCCc5ccc(OCc6ccc(F)c(C(F)(F)F)c6)c(CNCCO)c5)c4)cc3F)ccc21] |
| 234 | 918.15 | 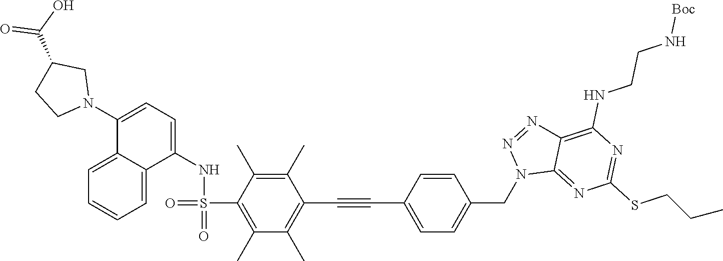 [SMILES: CCCSc1nc(NCCNC(=O)OC(C)(C)C)c2nnn(Cc3ccc(C#Cc4c(C)c(C)c(S(=O)(=O)Nc5ccc(N6CC[C@H](C(=O)O)C6)c6ccccc56)c(C)c4C)cc3)c2n1] |
| 235 | 924.53 | 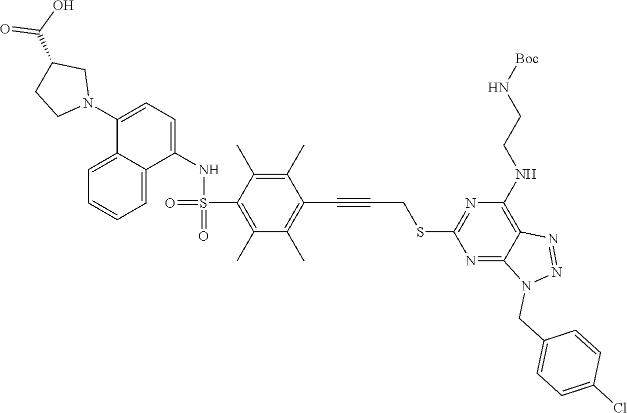 [SMILES: Cc1c(C)c(S(=O)(=O)Nc2ccc(N3CC[C@H](C(=O)O)C3)c3ccccc23)c(C)c(C)c1C#CCSc1nc(NCCNC(=O)OC(C)(C)C)c2nnn(Cc3ccc(Cl)cc3)c2n1] |
| 236 | 908.10 | 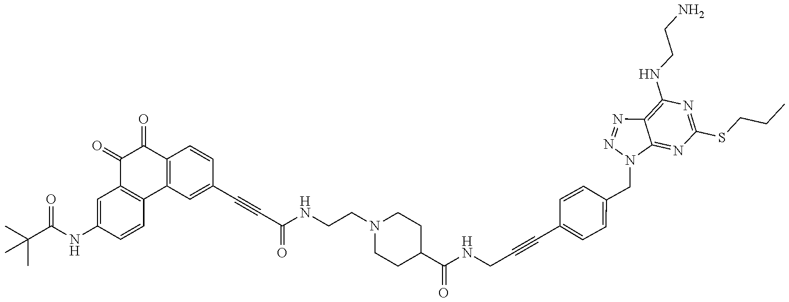 [SMILES: CCCSc1nc(NCCN)c2nnn(Cc3ccc(C#CCNC(=O)C4CCN(CCNC(=O)C#Cc5ccc6c(c5)-c5ccc(NC(=O)C(C)(C)C)cc5C(=O)C6=O)CC4)cc3)c2n1] |
| 237 | 916.16 | 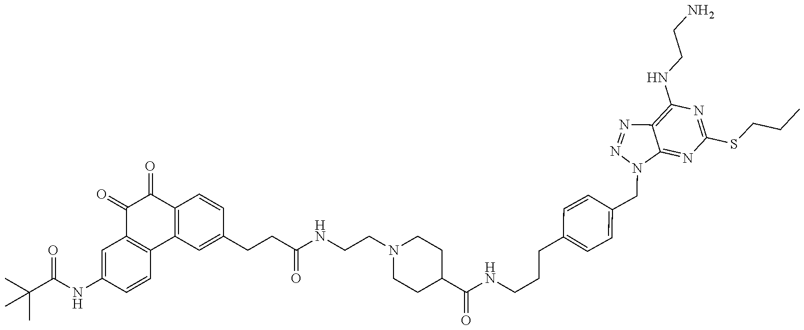 [SMILES: CCCSc1nc(NCCN)c2nnn(Cc3ccc(CCCNC(=O)C4CCN(CCNC(=O)CCc5ccc6c(c5)-c5ccc(NC(=O)C(C)(C)C)cc5C(=O)C6=O)CC4)cc3)c2n1] |
| 238 | 1212.52 | 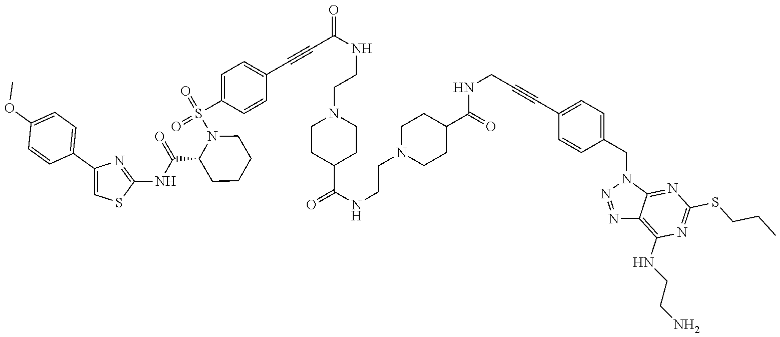 [SMILES: CCCSc1nc(NCCN)c2nnn(Cc3ccc(C#CCNC(=O)C4CCN(CCNC(=O)C5CCN(CCNC(=O)C#Cc6ccc(S(=O)(=O)N7CCCC[C@@H]7C(=O)Nc7nc(-c8ccc(OC)cc8)cs7)cc6)CC5)CC4)cc3)c2n1] |
| 239 | 1220.59 | 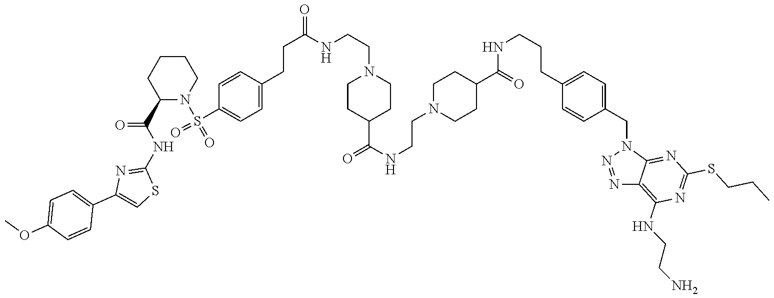 [SMILES: CCCSc1nc(NCCN)c2nnn(Cc3ccc(CCCNC(=O)C4CCN(CCNC(=O)C5CCN(CCNC(=O)CCc6ccc(S(=O)(=O)N7CCCC[C@@H]7C(=O)Nc7nc(-c8ccc(OC)cc8)cs7)cc6)CC5)CC4)cc3)c2n1] |
| 240 | 970.13 | 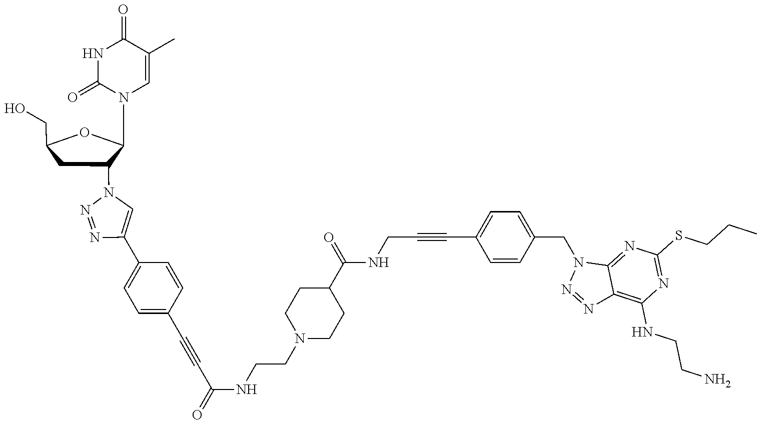 |
| 241 | 978.19 | 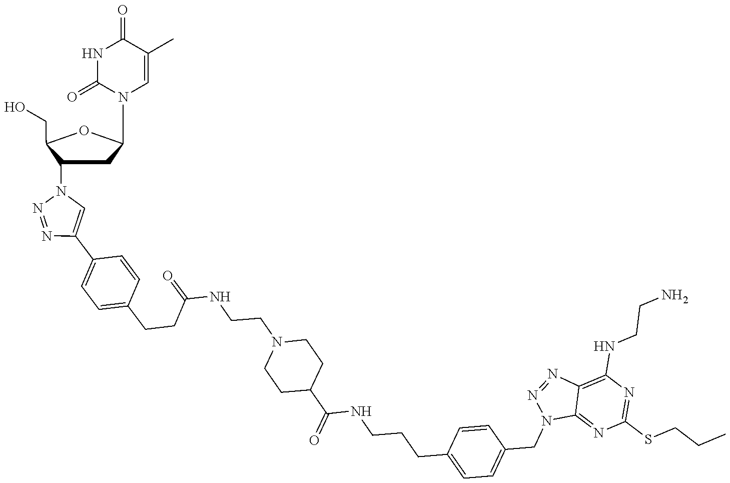 [SMILES: CCCSc1nc(NCCN)c2nnn(Cc3ccc(CCCNC(=O)C4CCN(CCNC(=O)CCc5ccc(-c6cn([C@@H]7C[C@H](n8cc(C)c(=O)[nH]c8=O)O[C@@H]7CO)nn6)cc5)CC4)cc3)c2n1] |

### Example 2. Representative Synthetic Schemes

### Synthesis of Intermediates

### Intermediate Scheme 1 Synthesis of Int-1, Int-2, Int-3, Int-4, Int-5, Int-6, and Int-7

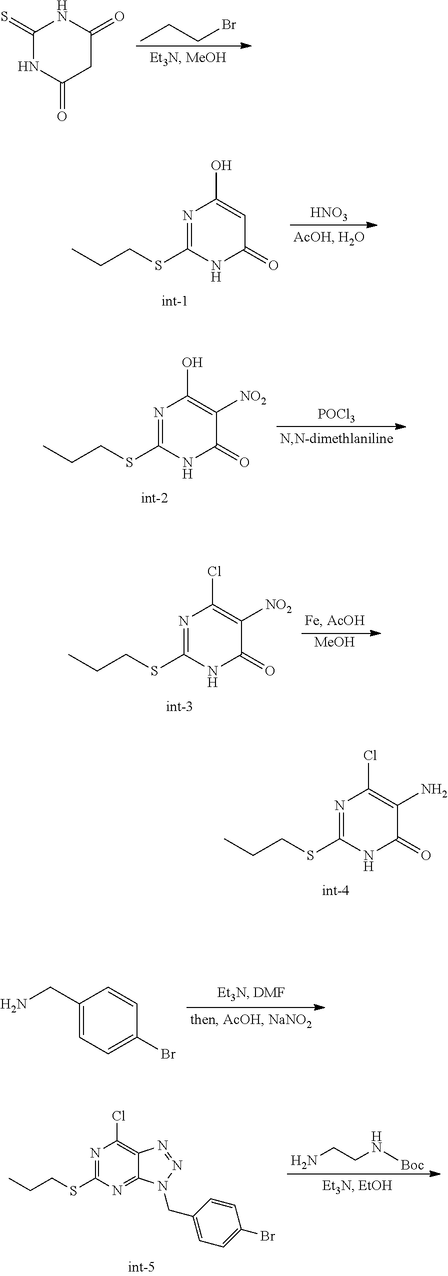

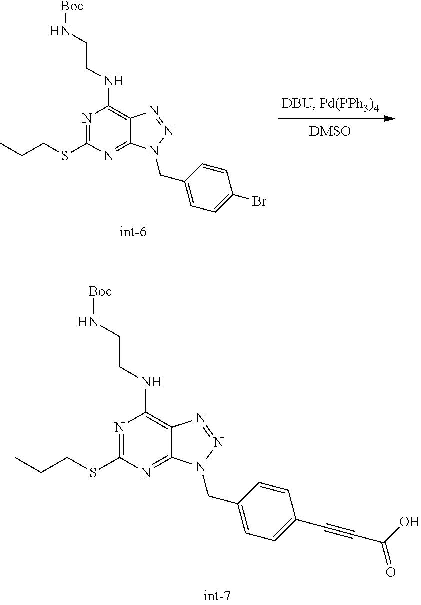 [SMILES: CCCSc1nc(NCCNC(=O)OC(C)(C)C)c2nnn(Cc3ccc(Br)cc3)c2n1 ; CCCSc1nc(NCCNC(=O)OC(C)(C)C)c2nnn(Cc3ccc(C#CC(=O)O)cc3)c2n1]

### Intermediate Scheme 2 Synthesis of Int-8, Int-9, Int-10, and Int-11

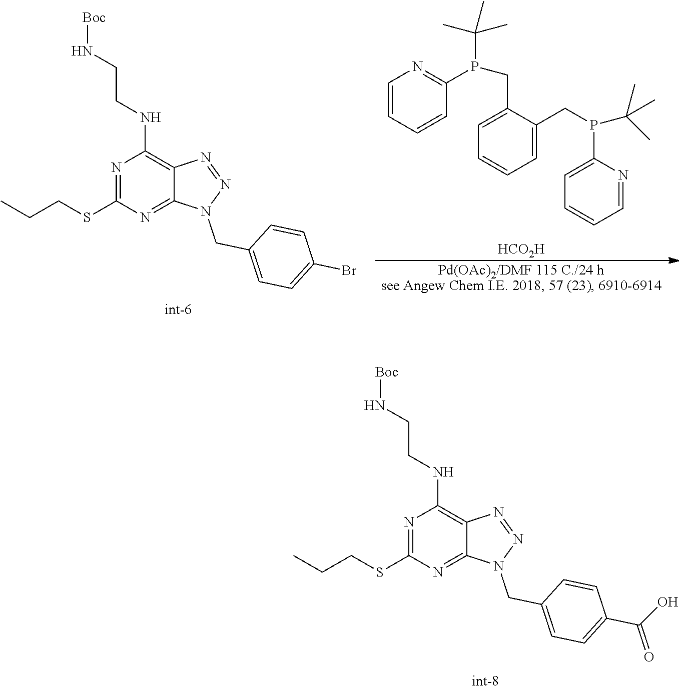 [SMILES: CCCSc1nc(NCCNC(=O)OC(C)(C)C)c2nnn(Cc3ccc(Br)cc3)c2n1 ; CC(C)(C)P(Cc1ccccc1CP(c1ccccn1)C(C)(C)C)c1ccccn1 ; "HCO2H" ; "Pd(OAc)2/DMF 115 C./24 hsee Angew Chem I.E. 2018, 57 (23), 6910-6914" ; CCCSc1nc(NCCNC(=O)OC(C)(C)C)c2nnn(Cc3ccc(C(=O)O)cc3)c2n1]

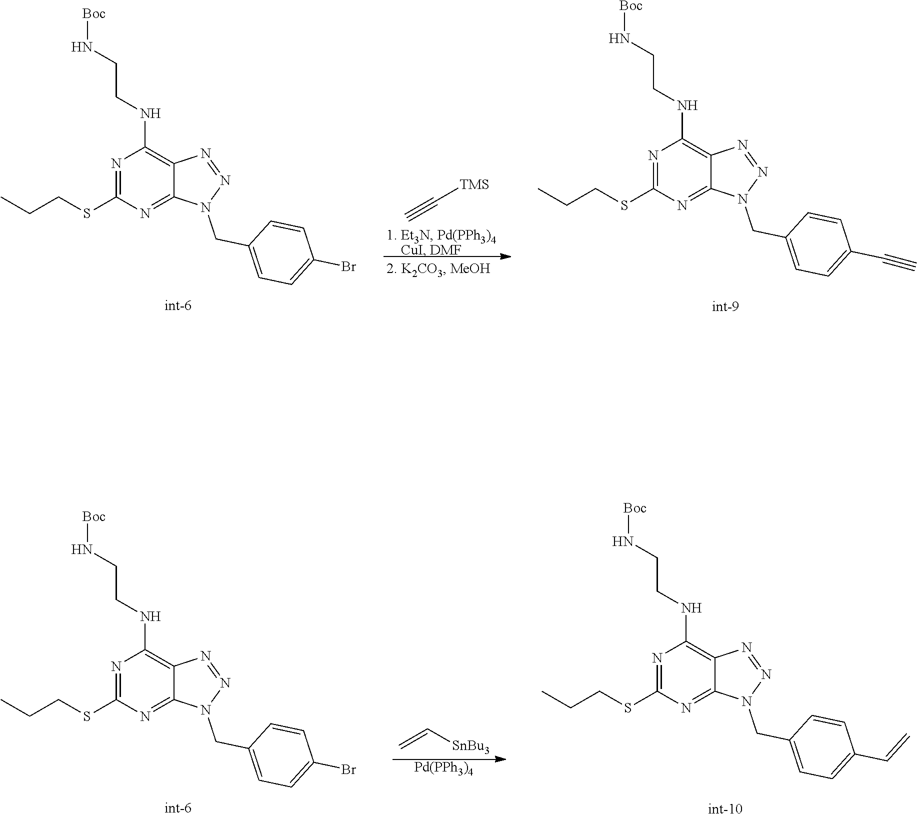

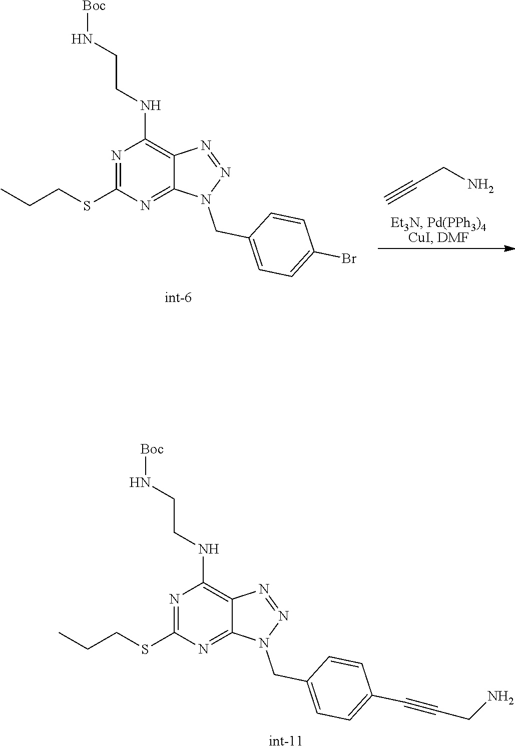

### Intermediate Scheme 3 Synthesis of Int-12, Int-13, Int-14, Int-15, Int-16, and Int-17

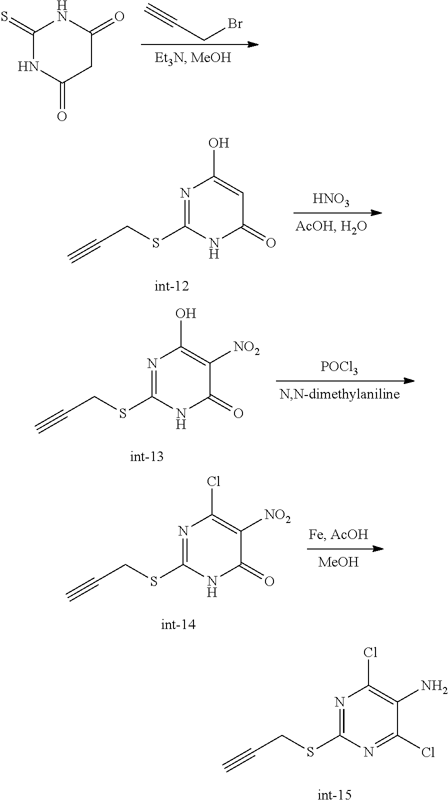

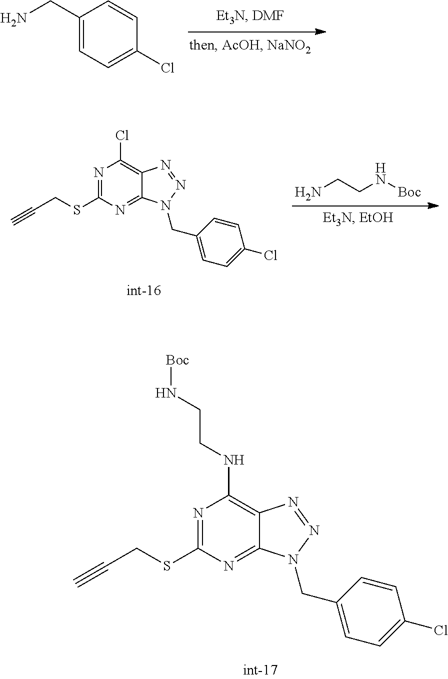 [SMILES: NCc1ccc(Cl)cc1 ; C#CCSc1nc(Cl)c2nnn(Cc3ccc(Cl)cc3)c2n1 ; CC(C)(C)OC(=O)NCCN ; C#CCSc1nc(NCCNC(=O)OC(C)(C)C)c2nnn(Cc3ccc(Cl)cc3)c2n1]

### Intermediate Scheme 4 Synthesis of Intermediates Int-18, Int-19, Int-20, Int-21, Int-22, and Int-23

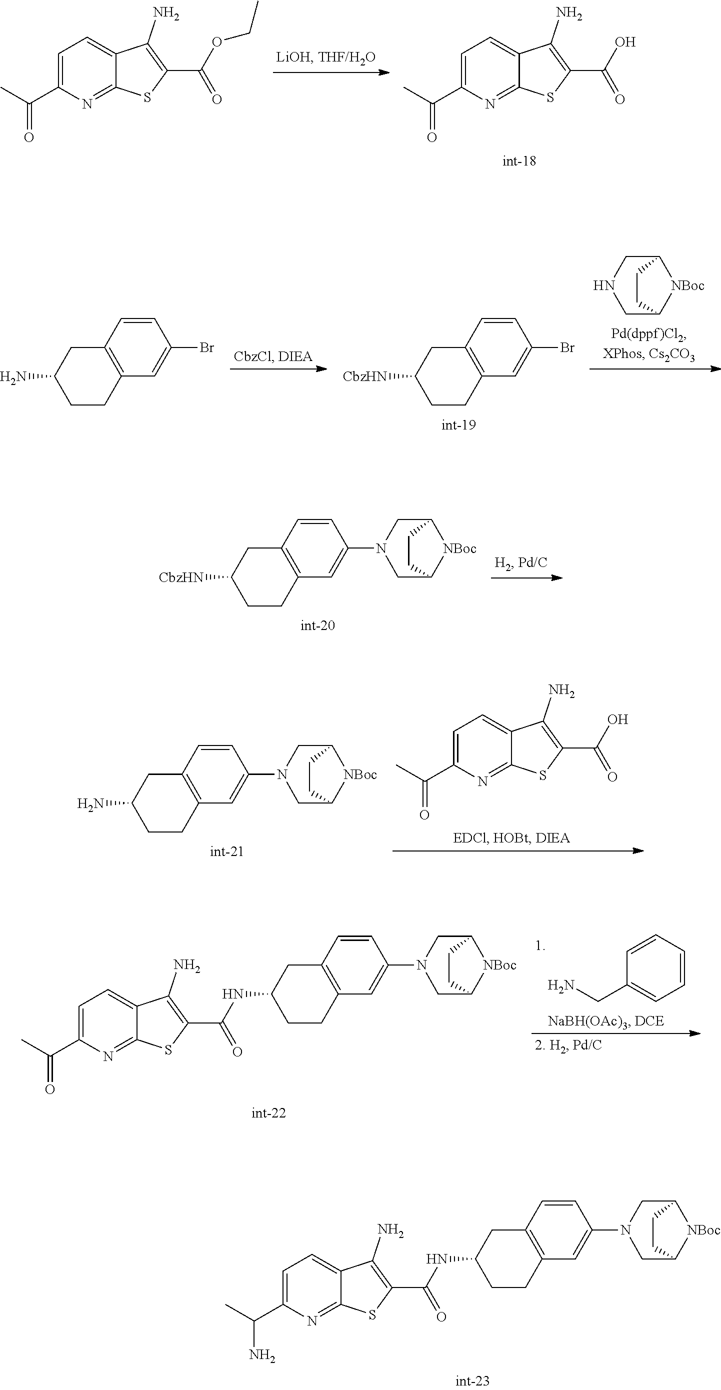

### Intermediate Scheme 5 Synthesis of Intermediates Int-24, Int-25, Int-26 1

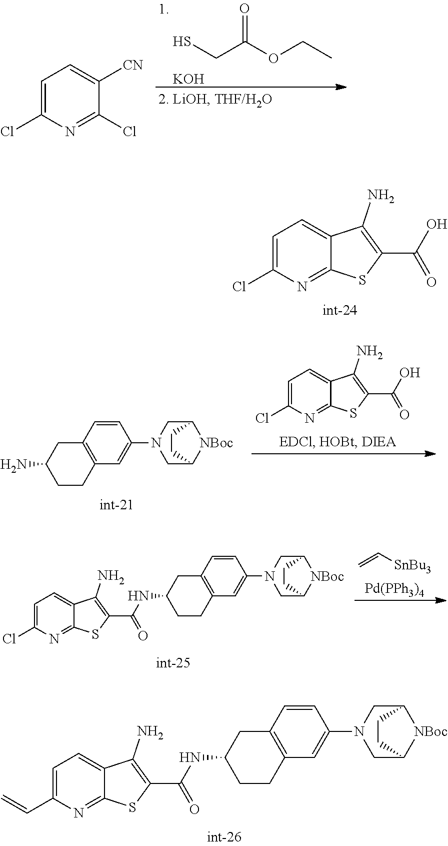

### Intermediate Scheme 6 Synthesis of Intermediates Int-27, Int-28, and Int-29

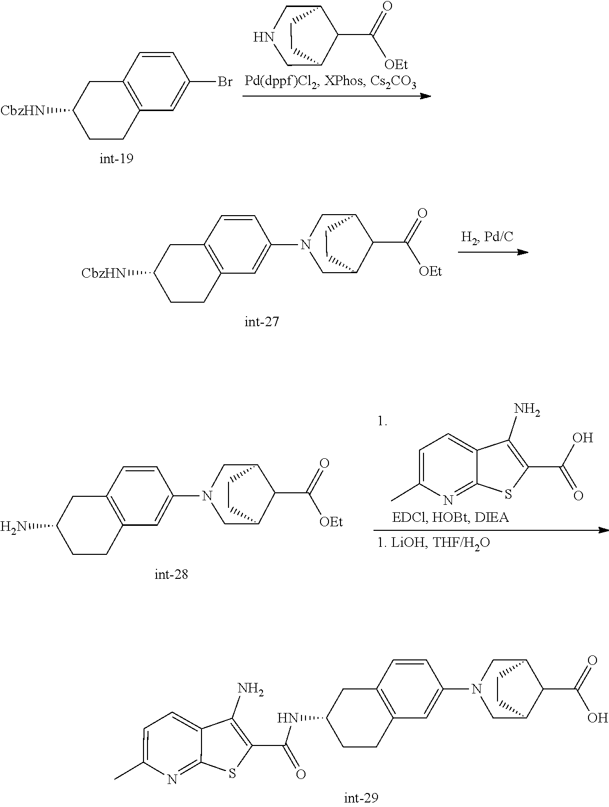 [SMILES: O=C(N[C@H]1CCc2cc(Br)ccc2C1)OCc1ccccc1 ; CCOC(=O)C1[C@@H]2CC[C@H]1CNC2 ; CCOC(=O)C1[C@@H]2CC[C@H]1CN(c1ccc3c(c1)CC[C@H](NC(=O)OCc1ccccc1)C3)C2 ; CCOC(=O)C1[C@@H]2CC[C@H]1CN(c1ccc3c(c1)CC[C@H](N)C3)C2 ; Cc1ccc2c(N)c(C(=O)O)sc2n1 ; Cc1ccc2c(N)c(C(=O)N[C@H]3CCc4cc(N5C[C@H]6CC[C@@H](C5)C6C(=O)O)ccc4C3)sc2n1]

### Intermediate Scheme 7 Synthesis of Intermediate Int-30

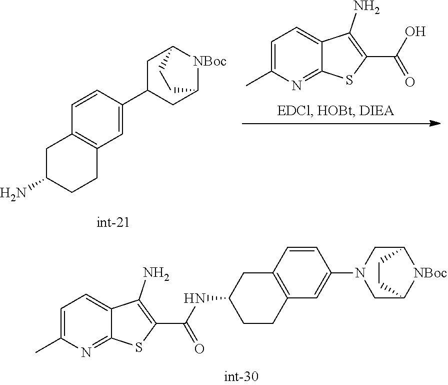 [SMILES: CC(C)(C)OC(=O)N1[C@@H]2CC[C@H]1CC(c1ccc3c(c1)CC[C@H](N)C3)C2 ; Cc1ccc2c(N)c(C(=O)O)sc2n1 ; Cc1ccc2c(N)c(C(=O)N[C@H]3CCc4cc(N5C[C@H]6CC[C@@H](C5)N6C(=O)OC(C)(C)C)ccc4C3)sc2n1]

### Intermediate Scheme 8 Synthesis of Intermediates Int-31, Int-32, Int-33, and Int-34

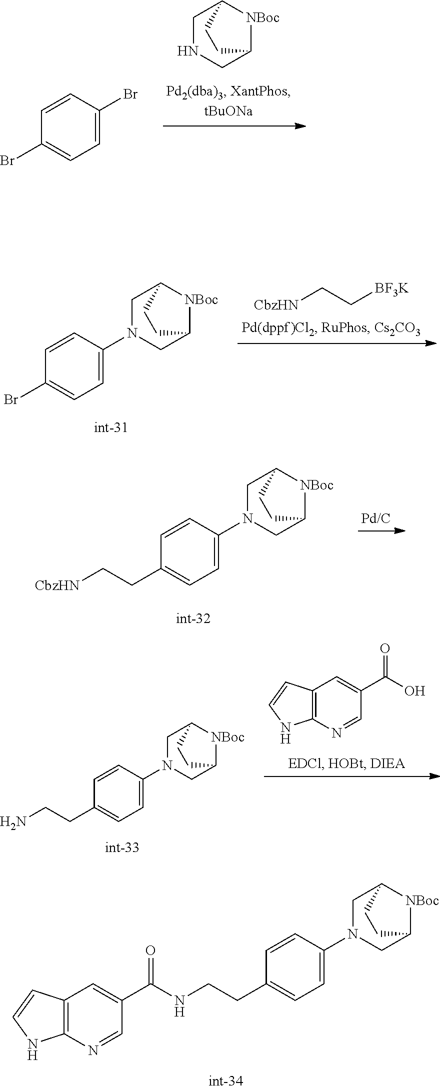

### Intermediate Scheme 9 Synthesis Intermediate Int-35

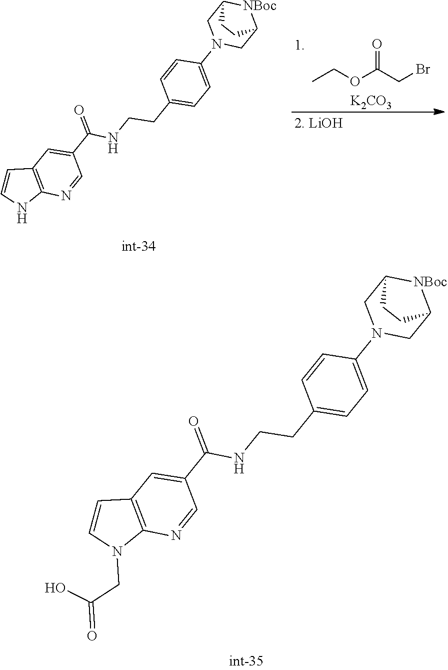

### Intermediate Scheme 10 Synthesis of Intermediates Int-36, Int-37 and Int-38

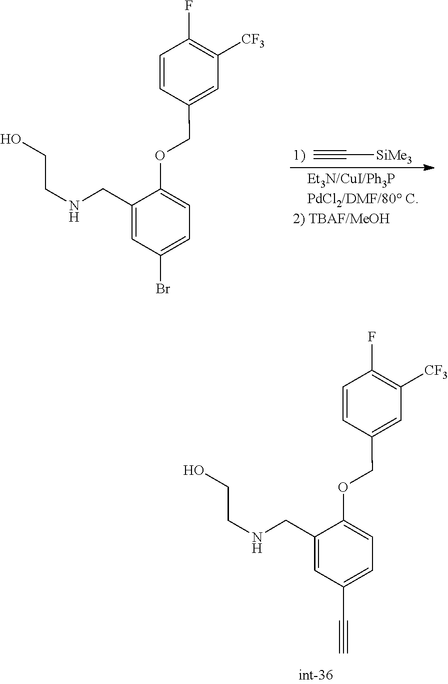

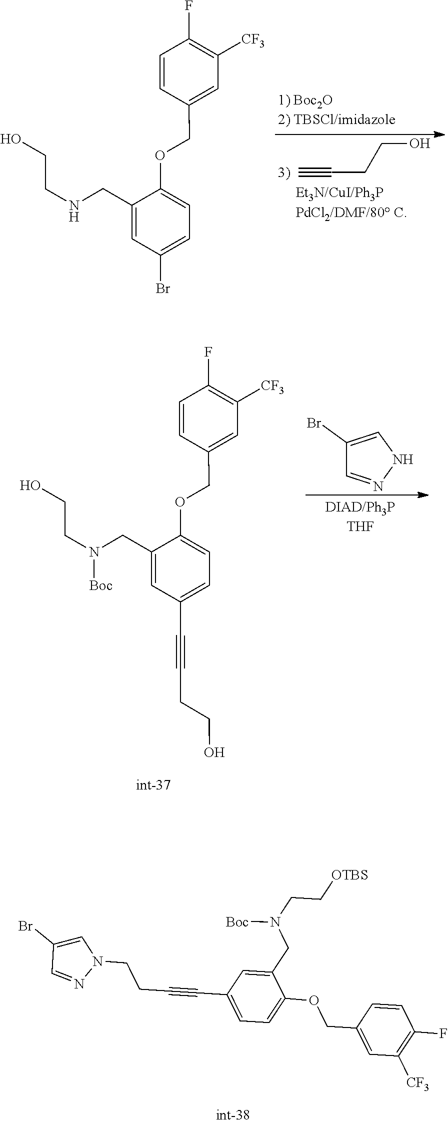

### Intermediate Scheme 11 Synthesis of Intermediates Int-39, Int-40, Int-41, Int-42, Int-43, Int-44, Int-45, Int-46

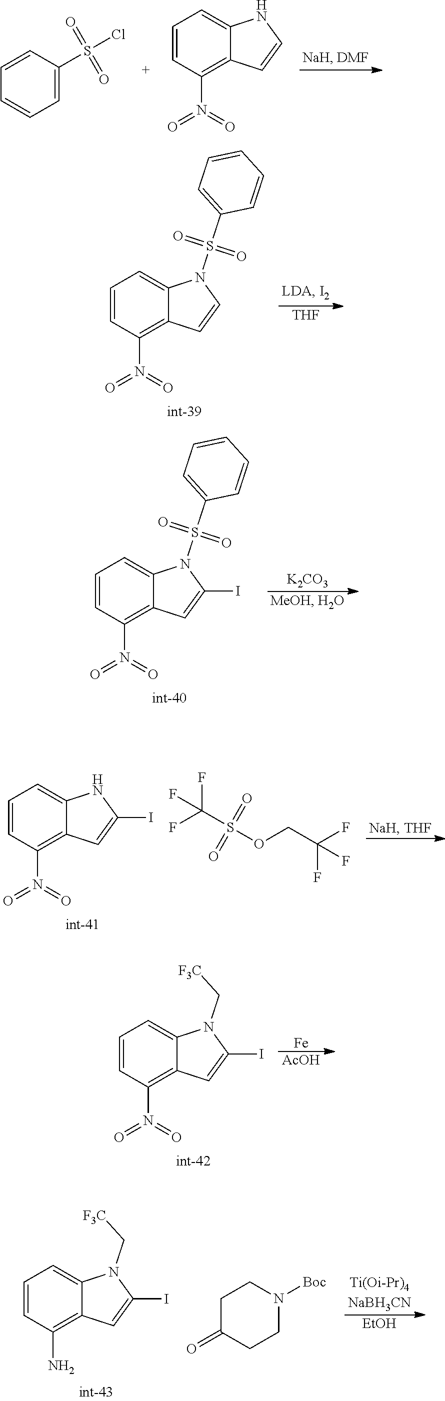

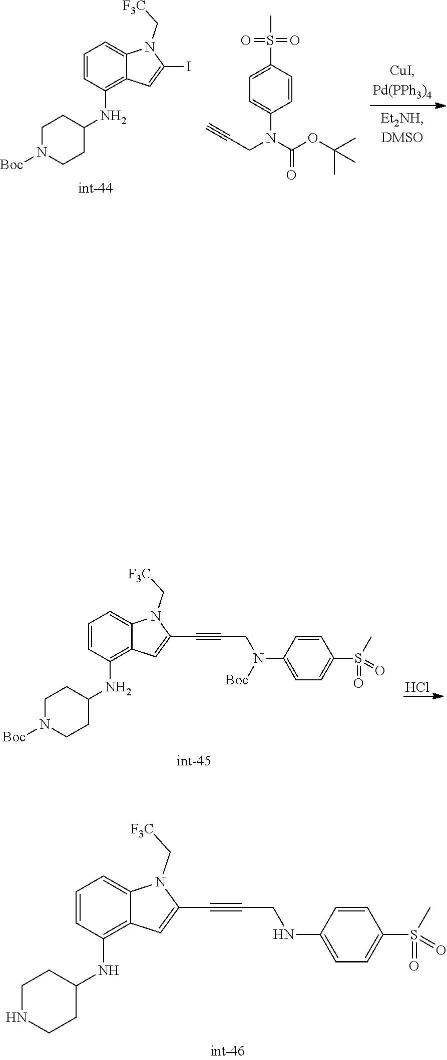 [SMILES: CC(C)(C)OC(=O)N1CCC(Nc2cccc3c2cc(I)n3CC(F)(F)F)CC1 ; C#CCN(C(=O)OC(C)(C)C)c1ccc(S(C)(=O)=O)cc1 ; CC(C)(C)OC(=O)N1CCC(Nc2cccc3c2cc(C#CCN(C(=O)OC(C)(C)C)c2ccc(S(C)(=O)=O)cc2)n3CC(F)(F)F)CC1 ; Cl ; CS(=O)(=O)c1ccc(NCC#Cc2cc3c(NC4CCNCC4)cccc3n2CC(F)(F)F)cc1]

### Intermediate Scheme 12 Synthesis of Intermediates Int-47, Int-48, Int-49, and Int-50

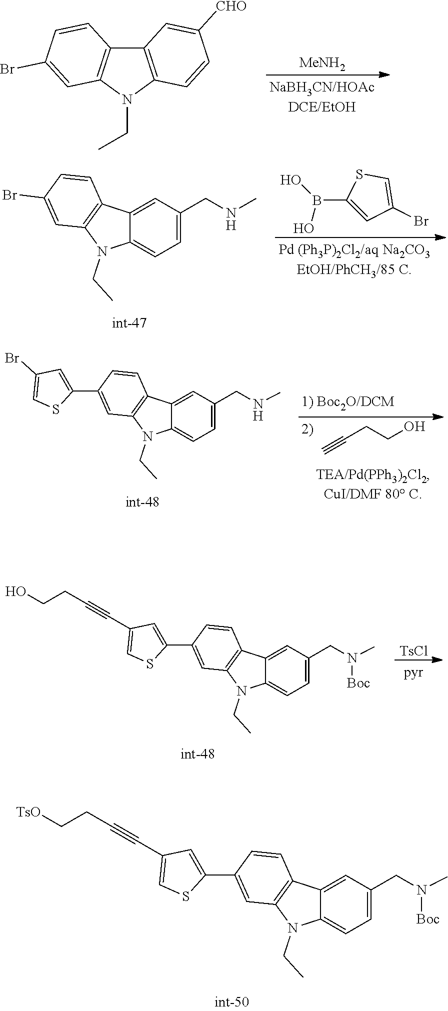

### Intermediate Scheme 13 Synthesis of Intermediates Int-51 and Int-52

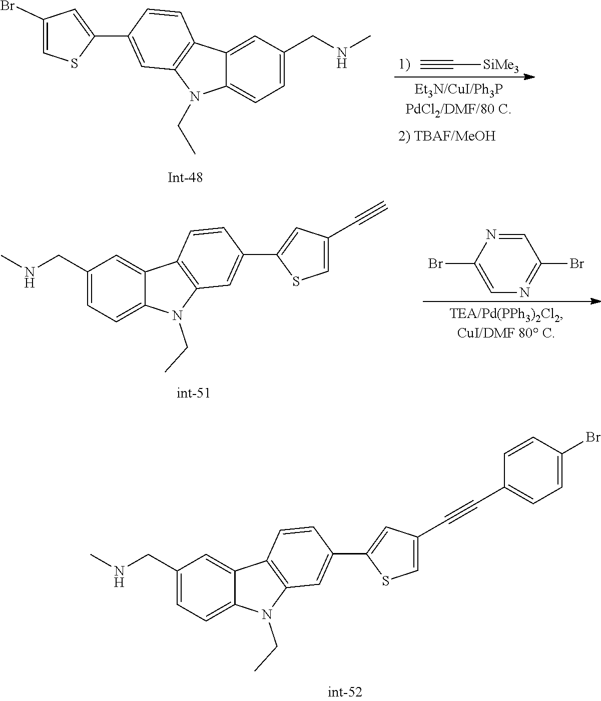

### Intermediate Scheme 14 Synthesis of Intermediates Int-53 and Int 54

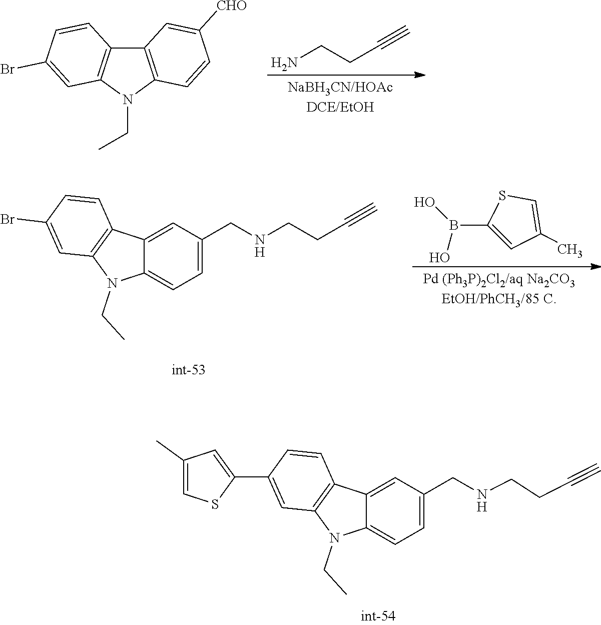

### Intermediate Scheme 15 Synthesis of Intermediates Int-55, Int-56, Int-57, Int-58, and Int-59

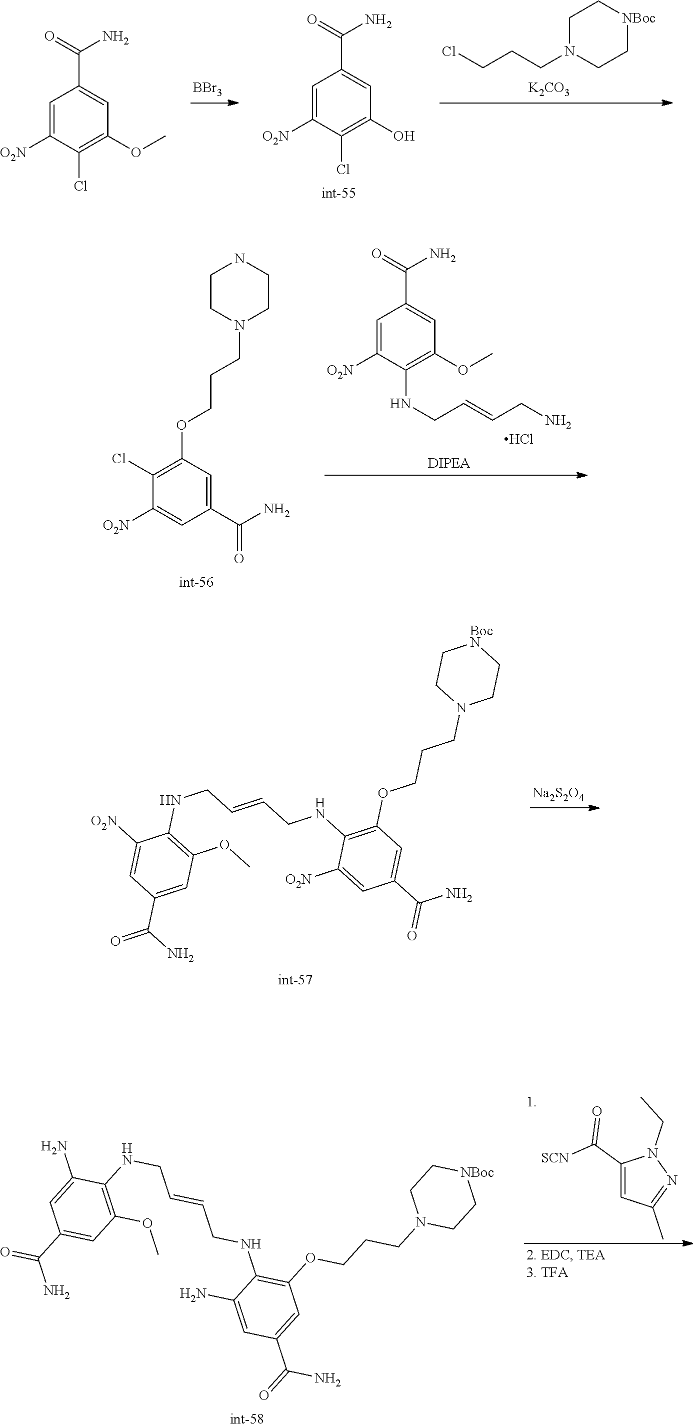

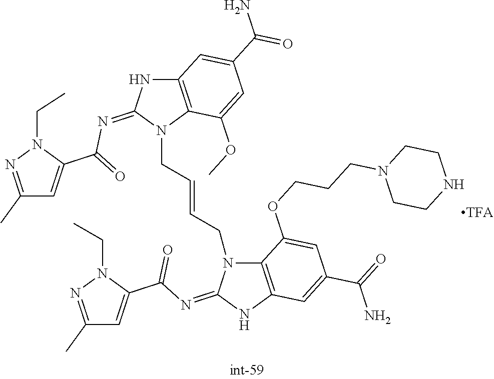 [SMILES: CCn1nc(C)cc1C(=O)/N=c1/[nH]c2cc(C(N)=O)cc(OC)c2n1C/C=C/Cn1/c(=N\C(=O)c2cc(C)nn2CC)[nH]c2cc(C(N)=O)cc(OCCCN3CCNCC3)c21]

### Intermediate Scheme 16 Synthesis of Intermediates Int-60 and Int-61

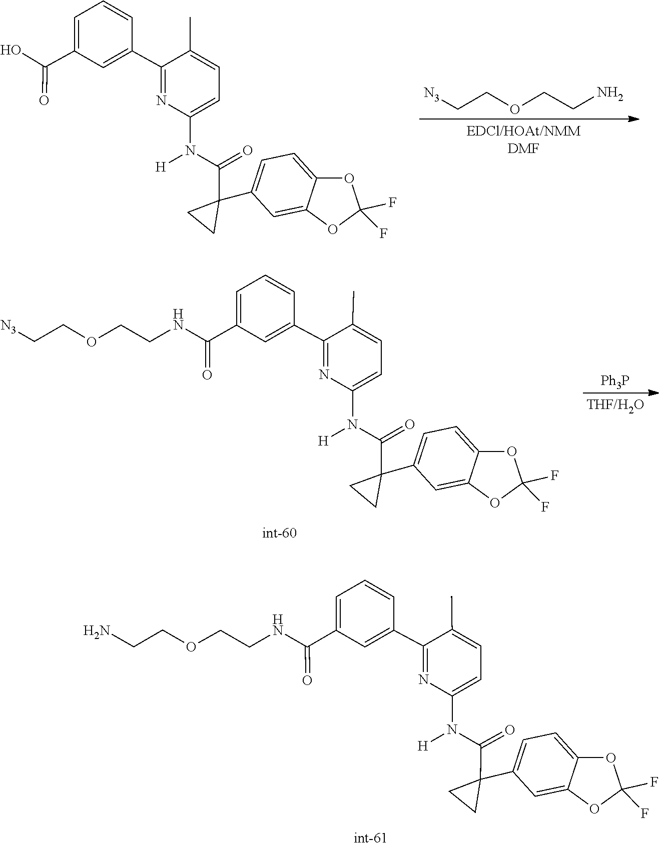 [SMILES: Cc1ccc(NC(=O)C2(c3ccc4c(c3)OC(F)(F)O4)CC2)nc1-c1cccc(C(=O)O)c1 ; [N-]=[N+]=NCCOCCN ; Cc1ccc(NC(=O)C2(c3ccc4c(c3)OC(F)(F)O4)CC2)nc1-c1cccc(C(=O)NCCOCCN=[N+]=[N-])c1 ; "Ph3P" ; Cc1ccc(NC(=O)C2(c3ccc4c(c3)OC(F)(F)O4)CC2)nc1-c1cccc(C(=O)NCCOCCN)c1]

### Intermediate Scheme 17 Synthesis of Intermediate Int-62

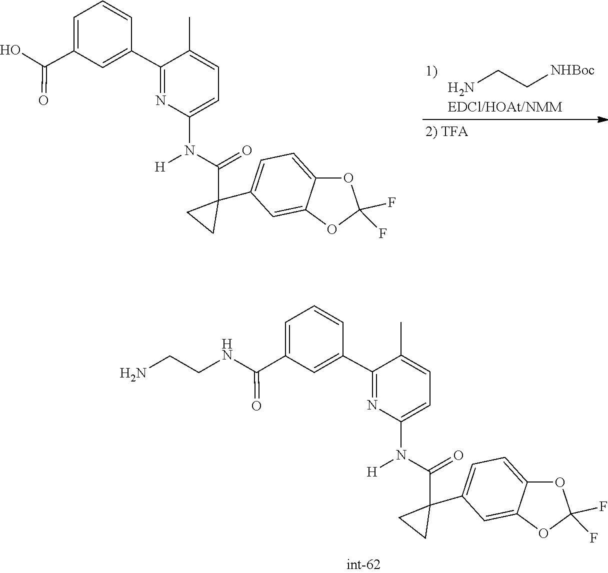 [SMILES: Cc1ccc(NC(=O)C2(c3ccc4c(c3)OC(F)(F)O4)CC2)nc1-c1cccc(C(=O)O)c1 ; CC(C)(C)OC(=O)NCCN ; Cc1ccc(NC(=O)C2(c3ccc4c(c3)OC(F)(F)O4)CC2)nc1-c1cccc(C(=O)NCCN)c1]

### Intermediate Scheme 17 Synthesis of Intermediate Int-63

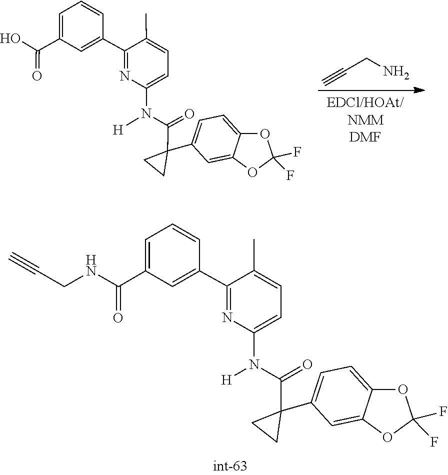

### Intermediate Scheme 19 Synthesis of Intermediate Int-64

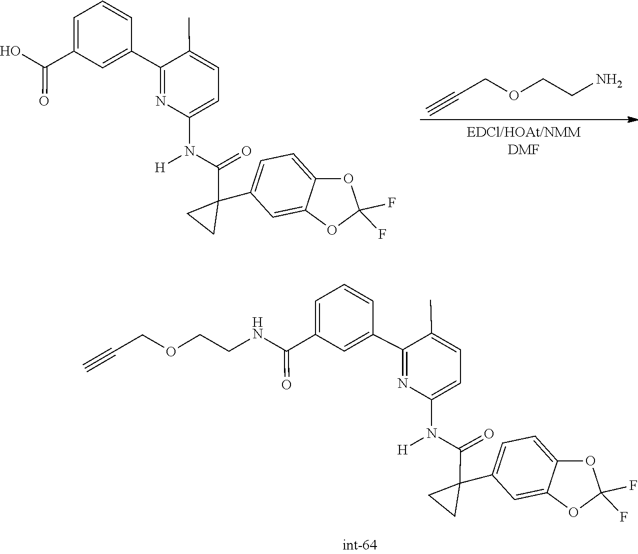

### Intermediate Scheme 20 Synthesis of Intermediate Int-65

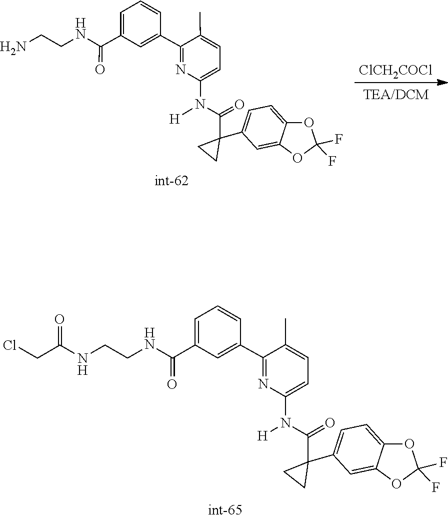 [SMILES: Cc1ccc(NC(=O)C2(c3ccc4c(c3)OC(F)(F)O4)CC2)nc1-c1cccc(C(=O)NCCN)c1 ; "ClCH2COCl" ; Cc1ccc(NC(=O)C2(c3ccc4c(c3)OC(F)(F)O4)CC2)nc1-c1cccc(C(=O)NCCNC(=O)CCl)c1]

### Intermediate Scheme 21 Synthesis of Intermediate Int-66

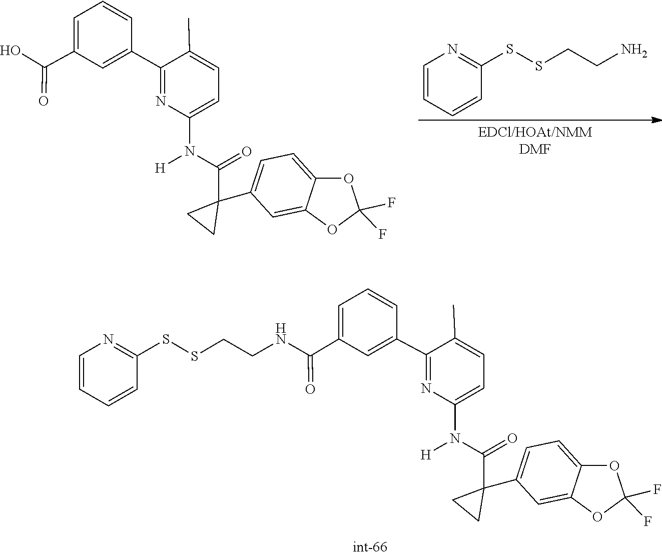 [SMILES: Cc1ccc(NC(=O)C2(c3ccc4c(c3)OC(F)(F)O4)CC2)nc1-c1cccc(C(=O)O)c1 ; NCCSSc1ccccn1 ; Cc1ccc(NC(=O)C2(c3ccc4c(c3)OC(F)(F)O4)CC2)nc1-c1cccc(C(=O)NCCSSc2ccccn2)c1]

### Intermediate Scheme 22 Synthesis of Intermediate Int-67

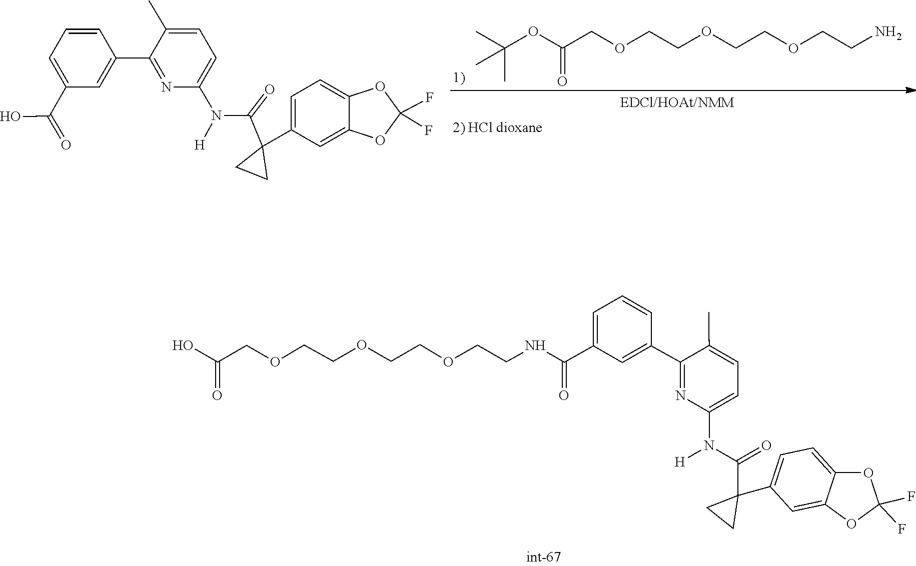 [SMILES: Cc1ccc(NC(=O)C2(c3ccc4c(c3)OC(F)(F)O4)CC2)nc1-c1cccc(C(=O)O)c1 ; CC(C)(C)OC(=O)COCCOCCOCCN ; Cc1ccc(NC(=O)C2(c3ccc4c(c3)OC(F)(F)O4)CC2)nc1-c1cccc(C(=O)NCCOCCOCCOCC(=O)O)c1]

### Intermediate Scheme 23 Synthesis of CFTR Intermediate Int-68

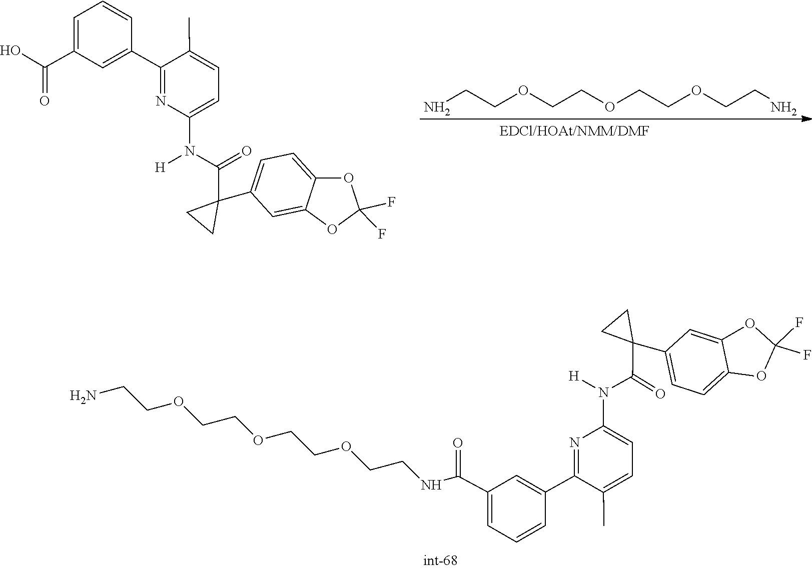 [SMILES: Cc1ccc(NC(=O)C2(c3ccc4c(c3)OC(F)(F)O4)CC2)nc1-c1cccc(C(=O)O)c1 ; NCCOCCOCCOCCN ; Cc1ccc(NC(=O)C2(c3ccc4c(c3)OC(F)(F)O4)CC2)nc1-c1cccc(C(=O)NCCOCCOCCOCCN)c1]

### Intermediate Scheme 24 Synthesis of Intermediate Int-68

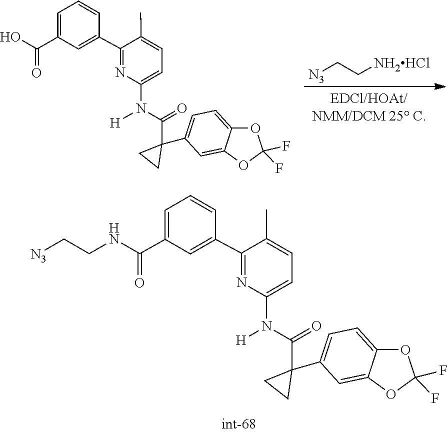

### Intermediate Scheme 25 Synthesis of Intermediate Int-69

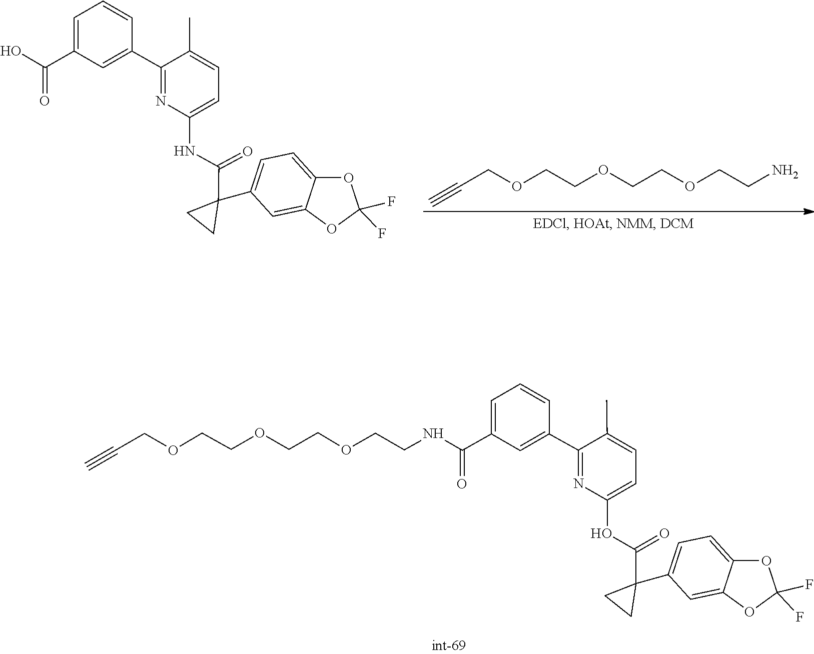

### Intermediate Scheme 26 Synthesis of Intermediates Int-70, Int-71, Int-72, Int-73, Int-74, and Int-75

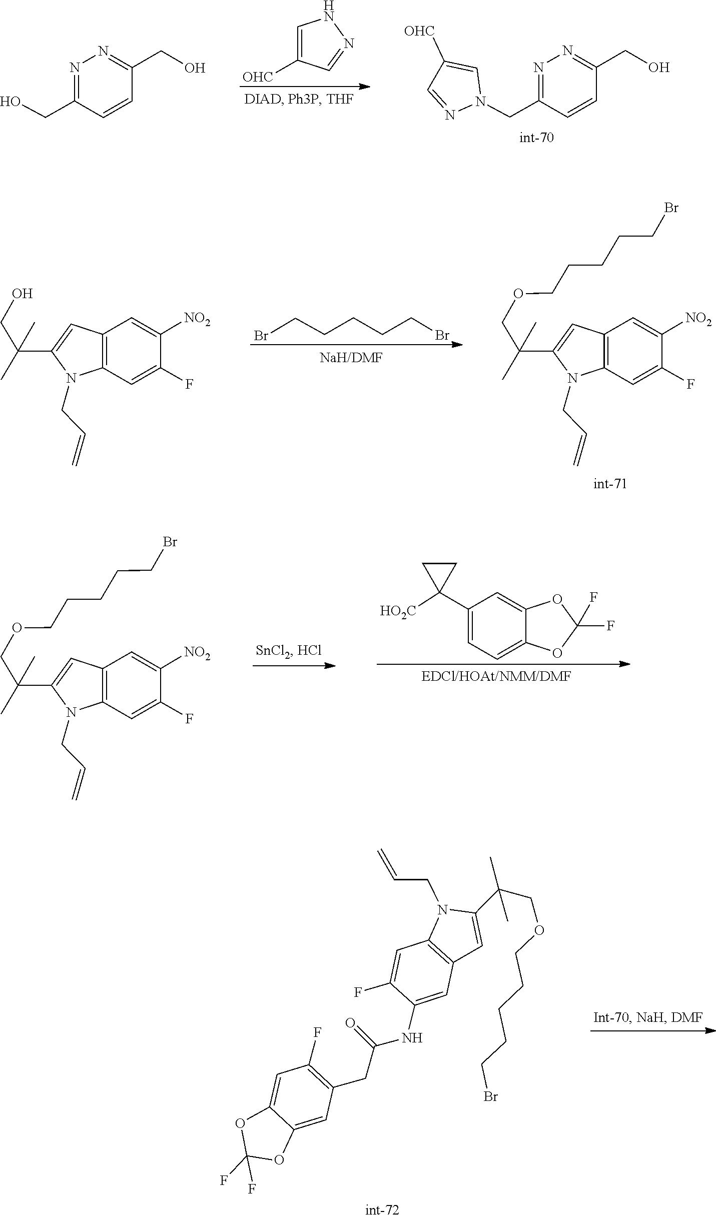

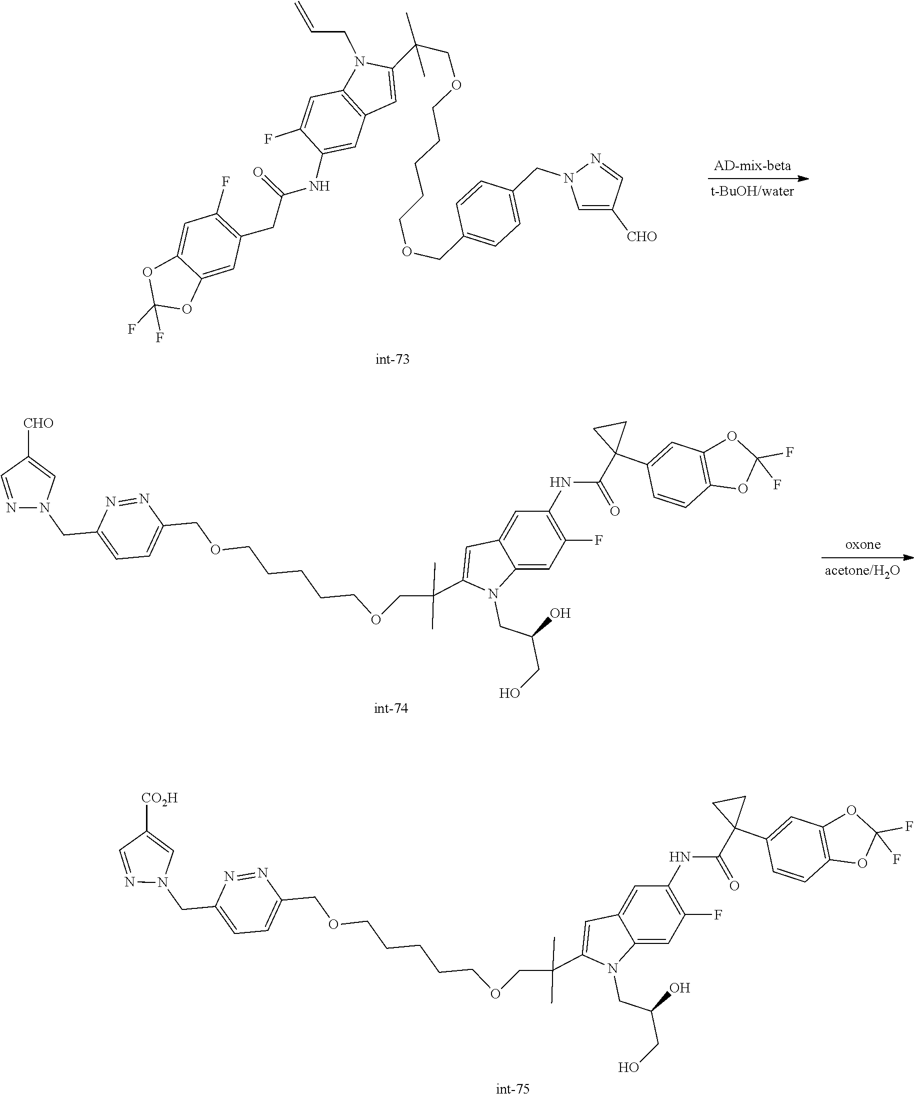 [SMILES: C=CCn1c(C(C)(C)COCCCCCOCc2ccc(Cn3cc(C=O)cn3)cc2)cc2cc(NC(=O)Cc3cc4c(cc3F)OC(F)(F)O4)c(F)cc21 ; "int-73" ; CC(C)(COCCCCCOCc1ccc(Cn2cc(C=O)cn2)nn1)c1cc2cc(NC(=O)C3(c4ccc5c(c4)OC(F)(F)O5)CC3)c(F)cc2n1C[C@@H](O)CO ; "int-74" ; CC(C)(COCCCCCOCc1ccc(Cn2cc(C(=O)O)cn2)nn1)c1cc2cc(NC(=O)C3(c4ccc5c(c4)OC(F)(F)O5)CC3)c(F)cc2n1C[C@@H](O)CO ; "int-75"]

### Intermediate Scheme 27 Synthesis of Intermediates Int-76, Int-77, Int-78 and Int-79

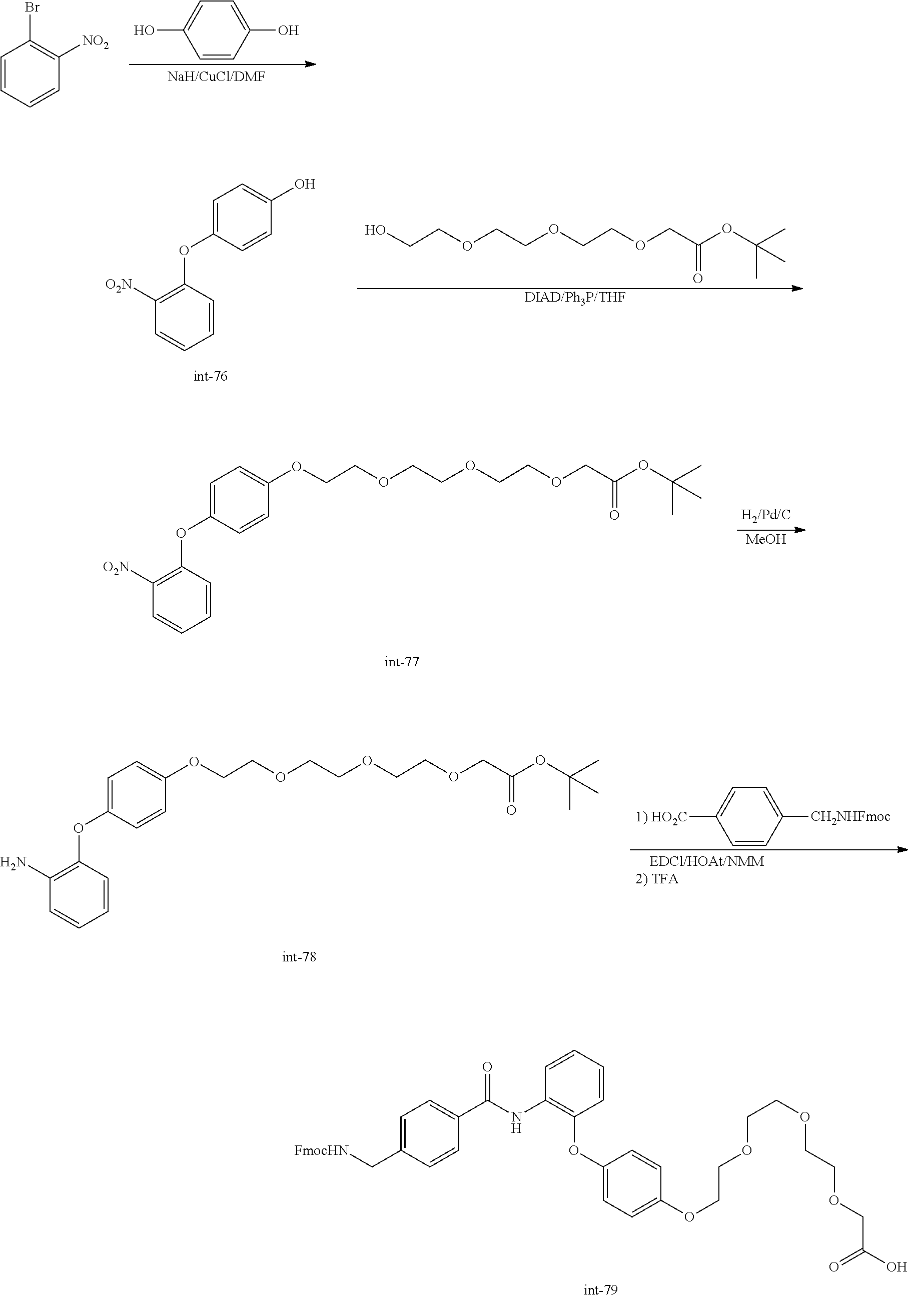

### Intermediate Scheme 28 Synthesis of Intermediates Int-80, Int-81, and Int-82

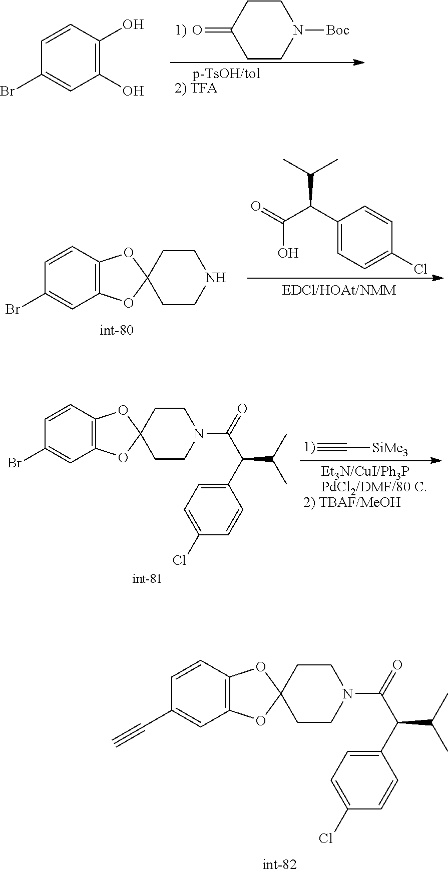

### Intermediate Scheme 29 Synthesis of Intermediates Int-83, Int-84, and Int-85

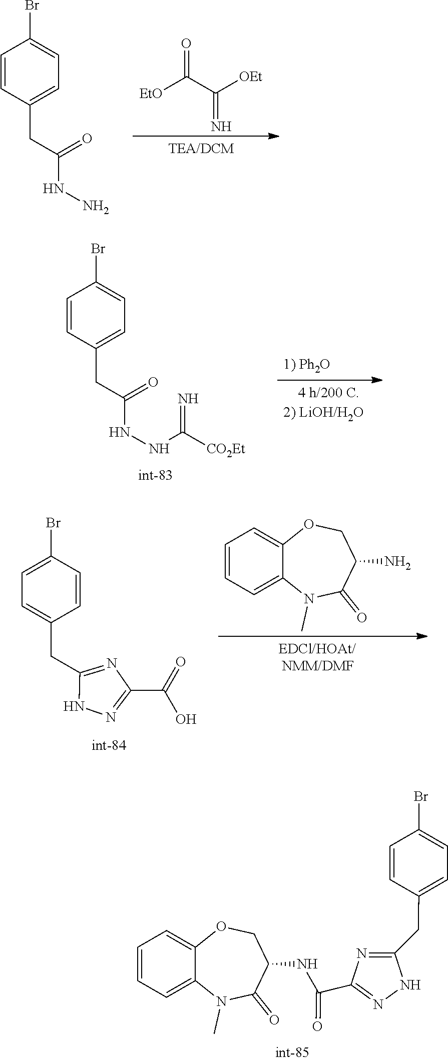

### Intermediate Scheme 30 Synthesis of Intermediate Int-86

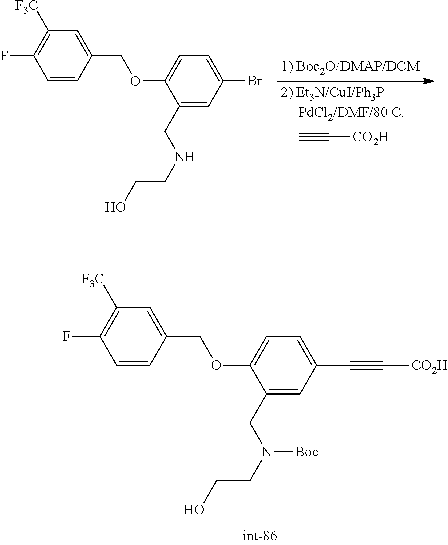

### Intermediate Scheme 31 Synthesis of Intermediates Int-87 and Int-88

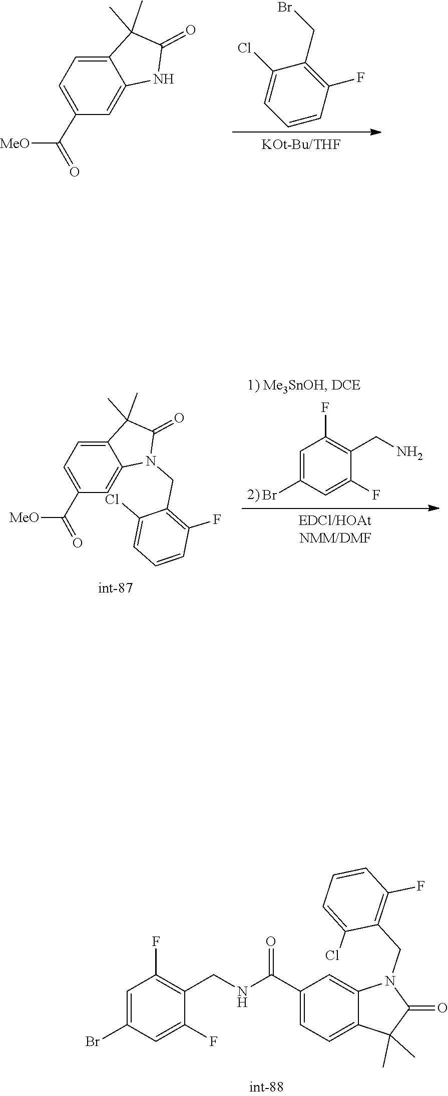 [SMILES: COC(=O)c1ccc2c(c1)NC(=O)C2(C)C ; Fc1cccc(Cl)c1CBr ; COC(=O)c1ccc2c(c1)N(Cc1c(F)cccc1Cl)C(=O)C2(C)C ; NCc1c(F)cc(Br)cc1F ; CC1(C)C(=O)N(Cc2c(F)cccc2Cl)c2cc(C(=O)NCc3c(F)cc(Br)cc3F)ccc21]

### Intermediate Scheme 32 Synthesis of Intermediates Int-89, Int-90, Int-91, Int-92

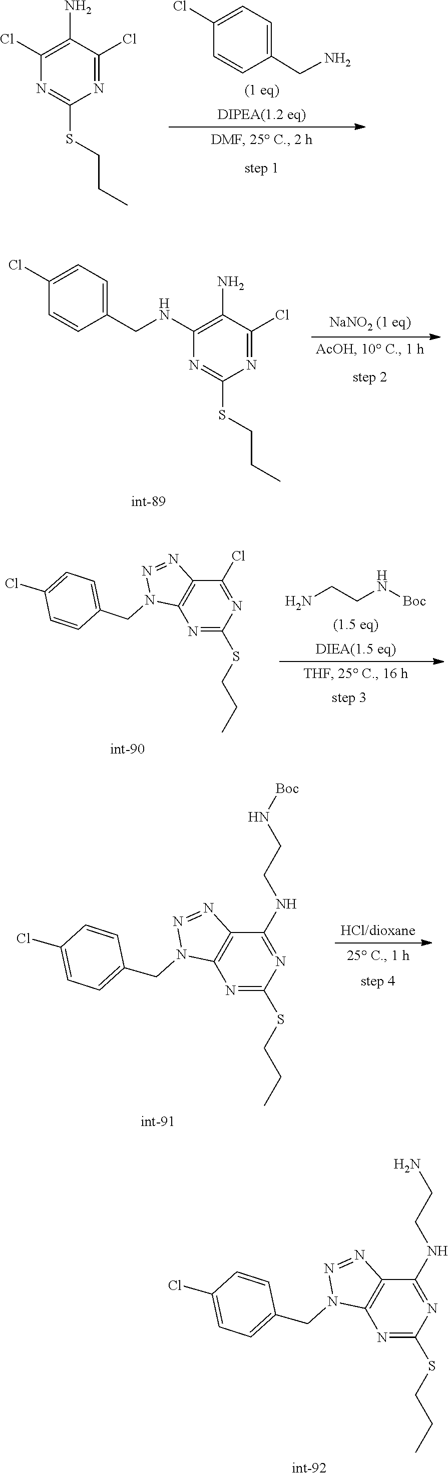

### Step 1: Synthesis of Int-89

Detailed Synthetic Procedure: To a solution of 4,6-dichloro-2-(propylthio)pyrimidin-5-amine (2 g, 8.40 mmol, 1 eq) and p-chlorobenzylamine (1.19 g, 8.40 mmol, 1.03 mL, 1 eq) in DMF (20 mL) was added DIPEA (1.30 g, 10.08 mmol, 1.76 mL, 1.2 eq). The mixture was stirred at 25° C. for 2 hr. LCMS showed 4,6-dichloro-2-(propylthio)pyrimidin-5-amine was consumed completely and one main peak with desired MW was detected, and TLC (PE:EA=3:1, Rf=0.2) showed a new spot was detected. The reaction mixture was diluted with water 100 mL and extracted with EA 45 mL (15 mL*3), dried over [Na₂SO₄], filtered and concentrated under reduced pressure to give a residue. The residue was purified by flash silica gel chromatography (ISCO®; 40 g SepaFlash® Silica Flash Column, Eluent of 25-35% Ethyl acetate/Petroleum ether gradient @40 mL/min) to give int-89 (1.60 g, 4.66 mmol, 55.49% yield, 100% purity) was obtained as a red solid, which was confirmed by LCMS and ¹H-NMR.

### Mass Found

Retention time=0.941 min, (M+H)=343.2, 5-95AB_R_220&254.M

Retention time=0.939 min, (M+H)=343.1, 5-95AB_R_220&254.M

### NMR Data

¹H NMR (400 MHz, CHLOROFORM-d) δ=7.23 (d, J=1.6 Hz, 4H), 5.94-5.71 (m, 1H), 4.69-4.59 (m, 2H), 3.02-2.96 (m, 2H), 1.74-1.59 (m, 2H), 0.99-0.94 (m, 3H).

### Step 2: Synthesis of Int-90

Detailed Synthetic Procedure: int-89 (0.5 g, 1.46 mmol, 1 eq) was dissolved in AcOH (5 mL) and cooled in an ice bath. An aqueous solution of NaNO₂ (111.70 mg, 1.62 mmol, 1 eq) was added dropwise, keeping the temperature no more than 10° C., the reaction was then stirred at 10° C. for 1 hr. TLC (PE:EA=3:1, Rf=0.6) showed starting material was consumed, and a new spot was detected. The reaction solution was dissolved in EA 5 mL, washed three times with water. And then neutralized with saturated sodium bicarbonate solution to neutral, washed, drying the organic phase. After evaporation, int-90 (0.47 g, 1.31 mmol, 89.69% yield, 98.468% purity) was obtained as a red oil, which was confirmed by LCMS and ¹H-NMR.

### Mass:

Retention time=1.025 min, (M+H)=354.0, 5-95AB_R_220&254.M

### NMR Data:

¹H NMR (400 MHz, CHLOROFORM-d) δ=7.43-7.36 (m, 2H), 7.36-7.28 (m, 2H), 5.74 (s, 2H), 3.21-3.16 (m, 2H), 1.83-1.77 (m, 2H), 1.11-1.07 (m, 3H).

### Step 3: Synthesis of Int-91

Detailed Synthetic Procedure: To a solution of int-90 (0.2 g, 564.56 μmol, 1 eq) and N-Boc-ethylenediamine (135.68 mg, 846.84 μmol, 133.02 μL, 1.5 eq) in THE (2 mL) was added DIEA (109.45 mg, 846.84 μmol, 147.50 μL, 1.5 eq). The mixture was stirred at 25° C. for 16 hr. LCMS showed int-90 was consumed completely and one main peak with desired MW was detected. The reaction mixture was concentrated under reduced pressure to remove solvent. The residue was diluted with water 3 mL and extracted with EA 9 mL (3 mL*3). The combined organic layers were dried over [Na₂SO₄], filtered and concentrated under reduced pressure to give the desired compound, and used in the next step directly without any purification. Int-91 (0.12 g, 251.04 μmol, 44.47% yield, 100% purity) was obtained as a white solid, which was confirmed by LCMS and ¹H-NMR.

### Mass:

Retention time=1.084 min, (M+H)=478.2, 5-95AB_R_220&254.lcm

Retention time=1.016 min, (M+H)=478.0, 5-95AB_R_220&254.M

### NMR Data:

¹H NMR (400 MHz, DMSO-d6) δ=8.92 (s, 1H), 7.43 (d, J=8.4 Hz, 2H), 7.38-7.30 (m, 2H), 6.90 (d, J=4.8 Hz, 1H), 5.69 (s, 2H), 3.23-3.17 (m, 2H), 3.12-3.05 (m, 2H), 1.72-1.61 (m, 2H), 1.41-1.27 (m, 9H), 1.21 (d, J=17.2 Hz, 2H), 1.00-0.95 (m, 3H).

### Step 4: Synthesis of Int-92

Detailed Synthetic Procedure: To a solution of int-91 (0.11 g, 230.12 μmol, 1 eq) in HCl/dioxane (1.1 mL). The mixture was stirred at 25° C. for 1 hr. LCMS showed int-91 was consumed completely and one main peak with desired MW was detected. The reaction mixture was concentrated to give the residue. The residue was purified by prep-HPLC (column: 3_Phenomenex Luna C18 75*30 mm*3 um; mobile phase: [water (HCl)-ACN]; B %: 32%-52%, 6 min) to give int-92 (0.0589 g, 142.15 μmol, 61.77% yield, 100% purity, HCl) was obtained as a yellow solid, which was confirmed by LCMS and ¹H-NMR.

### Mass:

Retention time=0.764 min, (M+H)=378.2, 5-95AB_R_220&254.M

Retention time=0.735 min, (M+H)=378.0, 5-95AB_R_220&254.M

### NMR Data:

1H NMR (400 MHz, DMSO-d6) δ=8.98-8.95 (m, 1H), 8.07-7.84 (m, 3H), 7.47-7.40 (m, 2H), 7.39-7.32 (m, 2H), 5.71 (s, 2H), 3.76-3.71 (m, 2H), 3.11-3.07 (m, 4H), 1.72-1.62 (m, 2H), 1.00-0.95 (m, 3H).

### Intermediate Scheme 33 Synthesis of Intermediates Int-93, Int-94, Int-95, Int-96

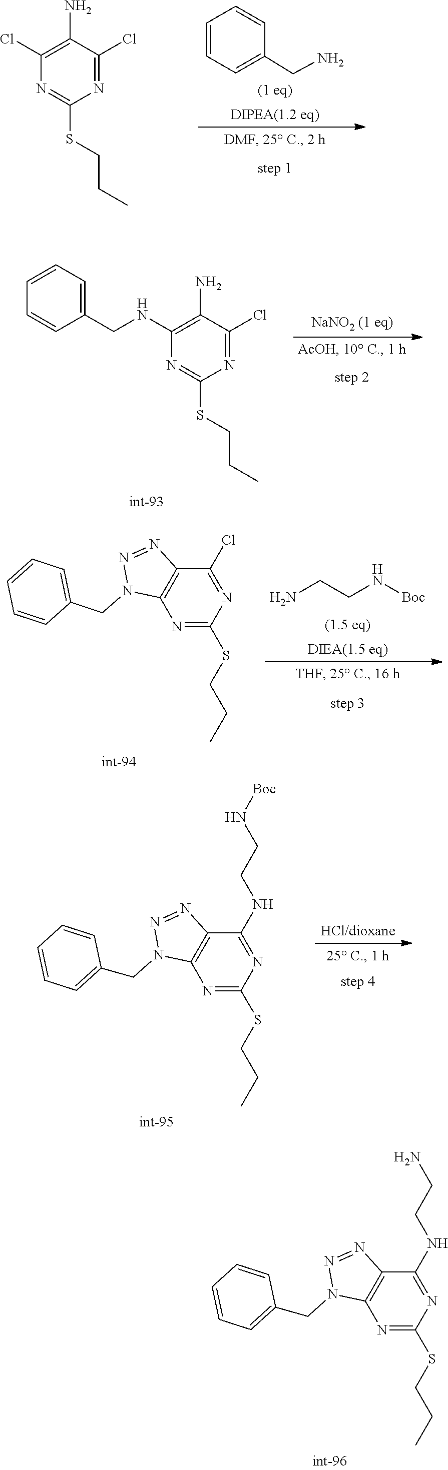

### Step 1: Synthesis of Int-93

Detailed Synthetic Procedure: To a solution of 4,6-dichloro-2-(propylthio)pyrimidin-5-amine (2 g, 8.40 mmol, 1 eq) and benzylamine (899.92 mg, 8.40 mmol, 915.49 μL, 1 eq) in DMF (20 mL) was added DIPEA (1.30 g, 10.08 mmol, 1.76 mL, 1.2 eq). The mixture was stirred at 25° C. for 2 hr. LCMS showed Reactant 1 was consumed completely and one main peak with desired MW was detected, and TLC (PE:EA=3:1, Rf=0.2) showed a new spot was detected. The reaction mixture was diluted with water 100 mL and extracted with EA 45 mL (15 mL*3), dried over [Na₂SO₄], filtered and concentrated under reduced pressure to give a residue. The residue was purified by flash silica gel chromatography (ISCO®; 40 g SepaFlash® Silica Flash Column, Eluent of 25˜35% Ethyl acetate/Petroleum ether gradient @40 mL/min) to give int-93 (1.55 g, 5.02 mmol, 59.76% yield, 100% purity) was obtained as a red solid, which was confirmed by LCMS and ¹H-NMR.

### Mass Found

Retention time=0.902 min, (M+H)=309.2, 5-95AB_R_220&254.M.

Retention time=0.907 min, (M+H)=309.1, 5-95AB_R_220&254.M.

### NMR Data

¹H NMR (400 MHz, CHLOROFORM-d) δ=7.39-7.28 (m, 5H), 5.65 (d, J=3.6 Hz, 1H), 4.68 (d, J=5.2 Hz, 2H), 3.05-3.00 (m, 2H), 1.75-1.66 (m, 2H), 1.01-0.96 (m, 3H).

### Step 2: Synthesis of Int-94

Detailed Synthetic Procedure: int-93 (0.5 g, 1.62 mmol, 1 eq) was dissolved in AcOH (5 mL) and cooled in an ice bath. An aqueous solution of NaNO₂ (111.70 mg, 1.62 mmol, 1 eq) was added dropwise, Keeping the temperature no more than 10° C., the reaction was then stirred at 10° C. for 1 hr. TLC (PE:EA=3:1, Rf=0.6) showed starting material was consumed and a new spot was detected. The reaction solution was dissolved in EA 5 mL, washed three times with water and then neutralized with saturated sodium bicarbonate solution to neutral, washed, drying the organic phase. After evaporation, int-94 (0.48 g, 1.44 mmol, 89.00% yield, 96-959% purity) was obtained as a red oil, which was confirmed by LCMS and ¹H-NMR.

### Mass:

Retention time=0.995 min, (M+H)=320.0, 5-95AB_R_220&254.M.

### NMR Data:

¹H NMR (400 MHz, CHLOROFORM-d) δ=7.52-7.41 (m, 2H), 7.40-7.30 (m, 3H), 5.78 (s, 2H), 3.22-3.16 (m, 2H), 1.83-1.77 (m, 2H), 1.12-1.04 (m, 3H).

### Step 3: Synthesis of Int-95

Detailed Synthetic Procedure: To a solution of int-94 (0.2 g, 625.37 μmol, 1 eq) and N-Boc-Ethylenediamine (150.29 mg, 938.06 μmol, 147.34 μL, 1.5 eq) in THE (2 mL) was added DIEA (121.23 mg, 938.06 μmol, 163.39 μL, 1.5 eq). The mixture was stirred at 25° C. for 16 hr. LCMS showed int-94 was consumed completely and one main peak with desired MW was detected. The reaction mixture was concentrated under reduced pressure to remove solvent. The residue was diluted with water 3 mL and extracted with EA 9 mL (3 mL*3). The combined organic layers were dried over Na₂SO₄, filtered and concentrated under reduced pressure to give the desired compound, and used in the next step directly without any purification. Int-95 (0.11 g, 247.99 μmol, 39.66% yield, 100% purity) was obtained as a white solid, which was confirmed by LCMS and ¹H-NMR.

### Mass:

Retention time=1.053 min, (M+H)=444.2, 5-95AB_R_220&254.lcm.

Retention time=0.986 min, (M+H)=444.1, 5-95AB_R_220&254.M.

### NMR Data:

¹H NMR (400 MHz, DMSO-d6) δ=8.95-8.86 (m, 1H), 7.37-7.28 (m, 5H), 6.91-6.88 (m, 1H), 5.67 (s, 2H), 3.24-3.17 (m, 2H), 3.12-3.05 (m, 2H), 1.72-1.62 (m, 2H), 1.42-1.27 (m, 9H), 1.25-1.13 (m, 2H), 1.00-0.95 (m, 3H).

### Step 4: Synthesis of Int-96

Detailed Synthetic Procedure: To a solution of int-95 (0.11 g, 247.99 μmol, 1 eq) in HCl/dioxane (1 mL). The mixture was stirred at 25° C. for 1 hr. LCMS showed int-95 was consumed completely and one main peak with desired MW was detected. The reaction mixture was concentrated to give the residue. The residue was purified by prep-HPLC (column: 3_Phenomenex Luna C18 75*30 mm*3 um; mobile phase: [water (HCl)-ACN]; B %: 27%-47%, 6 min) to give int-96 (0.05676 g, 149.40 μmol, 60.25% yield, 100% purity, HCl) was obtained as a yellow solid, which was confirmed by LCMS and ¹H-NMR.

### Mass:

Retention time=0.730 min, (M+H)=344.3, 5-95AB_R_220&254.M.

Retention time=0.708 min, (M+H)=344.1, 5-95AB_R_220&254.M.

### NMR Data:

¹H NMR (400 MHz, DMSO-d6) δ=8.97-8.94 (m, 1H), 7.97 (s, 3H), 7.40-7.27 (m, 5H), 5.70 (s, 2H), 3.77-3.71 (m, 2H), 3.14-3.05 (m, 4H), 1.73-1.63 (m, 2H), 1.01-0.97 (m, 3H).

### Intermediate Scheme 34 Synthesis of Intermediates Int-97 and Int-98

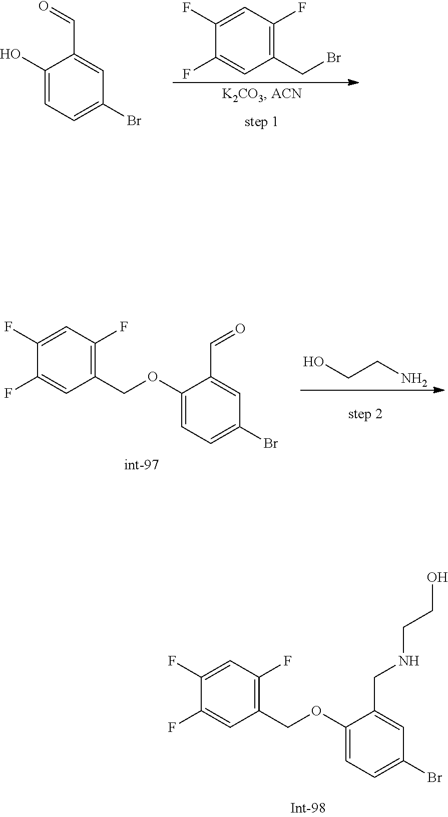

### Step 1: Synthesis of Intermediate 97

Detailed Synthetic Procedure: To a solution of 5-bromo-2-hydroxybenzaldehyde (1 g, 4.97 mmol, 1 eq) and K₂CO₃ (1.38 g, 9.95 mmol, 2 eq) in ACN (10 mL) was added 1-(bromomethyl)-2,4,5-trifluorobenzene (1.68 g, 7.46 mmol, 1.5 eq). The mixture was stirred at 60° C. for 12 hr. TLC indicated starting material was consumed completely and one new spot formed (PE:EA=5:1, Rf=0.70). The reaction mixture was concentrated under reduced pressure to give a residue, which was purified by column chromatography (SiO₂, EA/PE=0%˜35%) to get int-97 (700 mg, 2.03 mmol, 40.77% yield) as a white solid.

### NMR Data:

¹H NMR (400 MHz, CHLOROFORM-d) δ=10.33 (s, 1H), 7.88 (d, J=2.4 Hz, 1H), 7.59-7.56 (m, 1H), 7.31-7.22 (m, 1H), 6.97-6.87 (m, 2H), 5.10 (s, 2H)

### Step 2: Synthesis of Int-98

Detailed Synthetic Procedure: A solution of int-97 (50 mg, 144.88 μmol, 1 eq) and 2-aminoethanol (10.62 mg, 173.86 μmol, 10.51 μL, 1.2 eq) in MeOH (0.5 mL) was stirred at 25° C. for 0.5 hrs before NaBH₃CN (13.66 mg, 217.32 μmol, 1.5 eq) was added into the mixture. The mixture was stirred at 25° C. for 1 hr. LC-MS showed 43% of desired mass and no starting material was detected. The reaction mixture was concentrated under reduced pressure to give a residue, which was purified by reversed-phase column chromatography (0.1% FA condition). The eluent was concentrated and lyophilized to get int-98 (20 mg, 51.26 μmol, 35.38% yield, 100% purity) as a white solid.

### Mass:

Retention time: 0.692 min, (M+H)=389.9, 5-95AB_R_220&254.lcm.

Retention time: 0.480 min, (M+H)=389.7, 5-95AB_R_220&254.lcm.

### NMR Data:

¹H NMR (400 MHz, DMSO-d6) δ=8.24-8.18 (m, 1H), 7.75-7.67 (m, 1H), 7.67-7.59 (m, 1H), 7.56-7.50 (m, 1H), 7.44-7.39 (m, 1H), 7.10-7.07 (m, 1H), 5.13 (s, 2H), 3.73 (d, J=8.0 Hz, 2H), 3.49-3.44 (m, 2H), 2.61-2.56 (m, 2H)

### Intermediate Synthesis 35 Synthesis of Intermediates Int-99 and Int-100

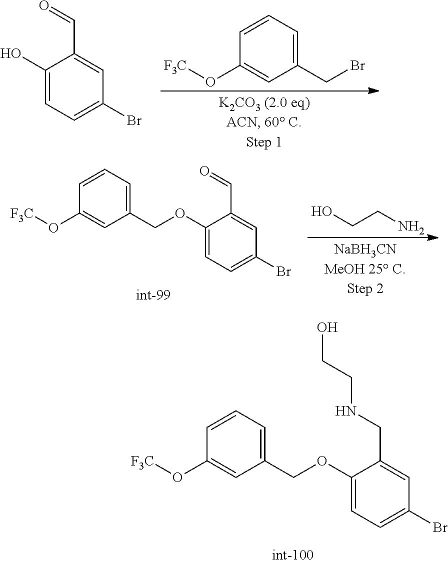

### Step 1: Synthesis of Int-99

Detailed Synthetic Procedure: To a solution of 5-bromo-2-hydroxybenzaldehyde (1 g, 4.97 mmol, 1 eq) in ACN (5 mL) was added K₂CO₃ (1.38 g, 9.95 mmol, 2 eq) at 25° C., then 1-(bromomethyl)-3-(trifluoromethoxy)benzene (1.90 g, 7.46 mmol, 1.21 mL, 1.5 eq) in ACN (5 mL) was added to the reaction mixture. The mixture was stirred at 60° C. for 12 hrs. LCMS showed 66.16% desired molecular weight was detected. The solvent was filtered and evaporated under reduced pressure to give crude product. The residue was purified by column chromatography system (SiO₂, Petroleum ether/Ethyl acetate=100/0 to 5/1, Rf=0.70) to give int-99 (1.6 g, 3.96 mmol, 79.60% yield, 92.83% purity) as yellow oil and confirmed by HNMR.

### Mass Found

LCMS: Retention time: 0.735 min, (M+H)=376.7, 5-95AB_1 min·1 cm.

### NMR Data

¹H NMR (400 MHz, CHLOROFORM-d) δ=10.47 (s, 1H), 7.98 (d, J=2.4 Hz, 1H), 7.66-7.62 (m, 1H), 7.49-7.43 (m, 1H), 7.37 (d, J=8.0 Hz, 1H), 7.30 (s, 1H), 7.24 (d, J=8.0 Hz, 1H), 6.94 (d, J=8.8 Hz, 1H), 5.20 (s, 2H).

### Step 2: Synthesis of Int-100

Detailed Synthetic Procedure: To a solution of int-99 (0.2 g, 533.14 μmol, 1 eq) in MeOH (2 mL) was added 2-aminoethanol (39.08 mg, 639.77 μmol, 38.69 μL, 1.2 eq), the mixture was stirred at 25° C. for 30 min. Then NaBH₃CN (50.26 mg, 799.71 μmol, 1.5 eq) was added to the mixture. The mixture was stirred at 25° C. for 1 hr. LCMS showed 63.25% desired molecular weight was detected. The mixture was concentrated to give crude product. The crude product was purified by reverse-phase (0.1% FA condition) and lyophilized to give int-100 (200 mg, 473.63 μmol, 88.84% yield, 99.51% purity) as white solid which was confirmed by LCMS, ¹H-NMR and ¹⁹F-NMR.

### Mass Found

LCMS: Retention time: 0.498 min, (M+H)=421.8, 5-95AB_1 min·1 cm

LCMS: Retention time: 0.505 min, (M+H)=421.7, 5-95AB_1 min·1 cm

### NMR Data

¹H NMR (400 MHz, DMSO-d6) δ=8.20 (s, 1H), 7.56-7.49 (m, 3H), 7.46 (s, 1H), 7.40-7.37 (m, 1H), 7.33 (d, J=8.0 Hz, 1H), 7.01 (d, J=8.8 Hz, 1H), 5.20 (s, 2H), 3.75 (s, 2H), 3.47 (s, 2H), 2.61-2.57 (m, 2H).

### Intermediate Synthesis 36 Synthesis of Intermediates Int-101 and Int-102

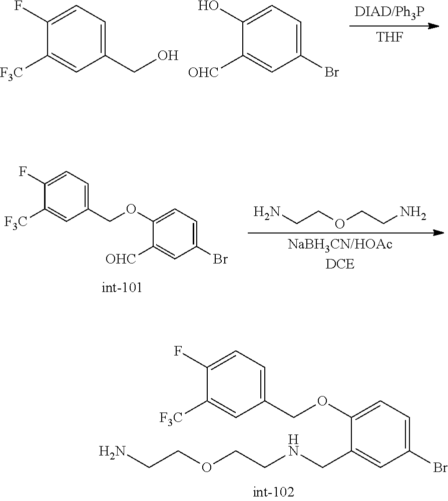 [SMILES: OCc1ccc(F)c(C(F)(F)F)c1 ; O=Cc1cc(Br)ccc1O ; O=Cc1cc(Br)ccc1OCc1ccc(F)c(C(F)(F)F)c1 ; NCCOCCN ; NCCOCCNCc1cc(Br)ccc1OCc1ccc(F)c(C(F)(F)F)c1 ; "THF"]

### Intermediate Synthesis 37 Synthesis of Intermediates Int-103

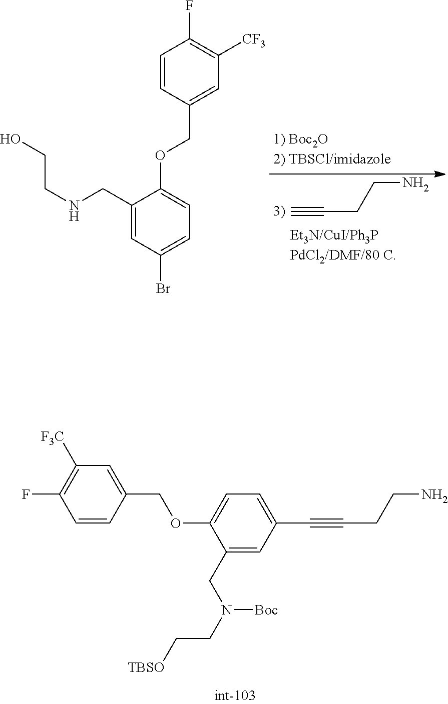

### Intermediate Synthesis 38 Synthesis of Intermediates Int-104 and Int-105

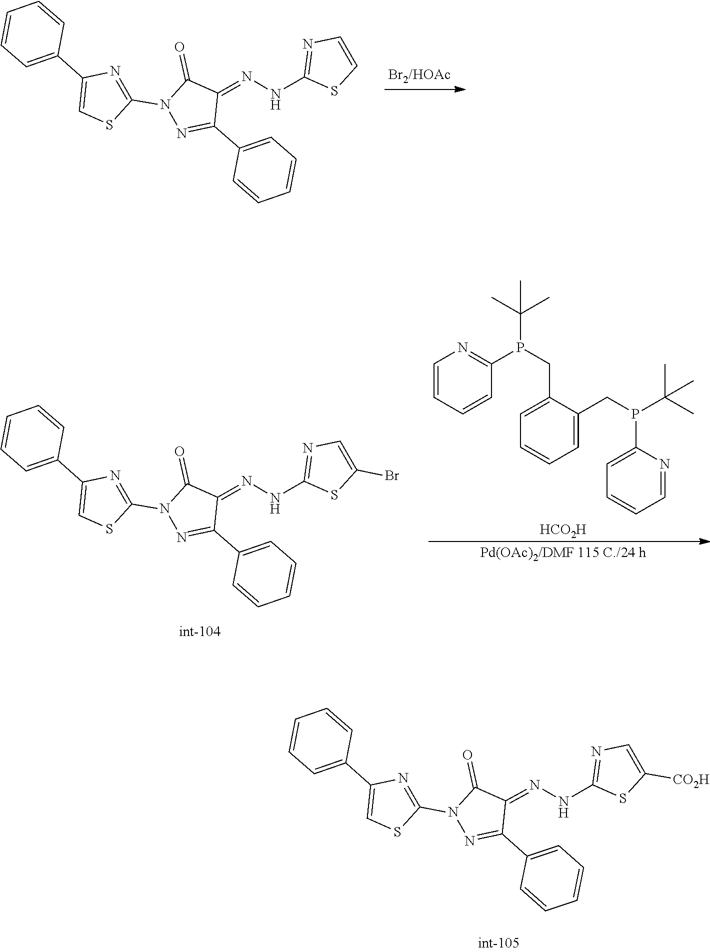 [SMILES: O=C1/C(=N/Nc2nccs2)C(c2ccccc2)=NN1c1nc(-c2ccccc2)cs1 ; O=C1/C(=N/Nc2ncc(Br)s2)C(c2ccccc2)=NN1c1nc(-c2ccccc2)cs1 ; CC(C)(C)P(Cc1ccccc1CP(c1ccccn1)C(C)(C)C)c1ccccn1 ; "Pd(OAc)2/DMF 115 C./24 h" ; "HCO2H" ; O=C(O)c1cnc(N/N=C2/C(=O)N(c3nc(-c4ccccc4)cs3)N=C2c2ccccc2)s1]

### Intermediate Synthesis 39 Synthesis of Intermediates Int-106, Int-107 and Int-108

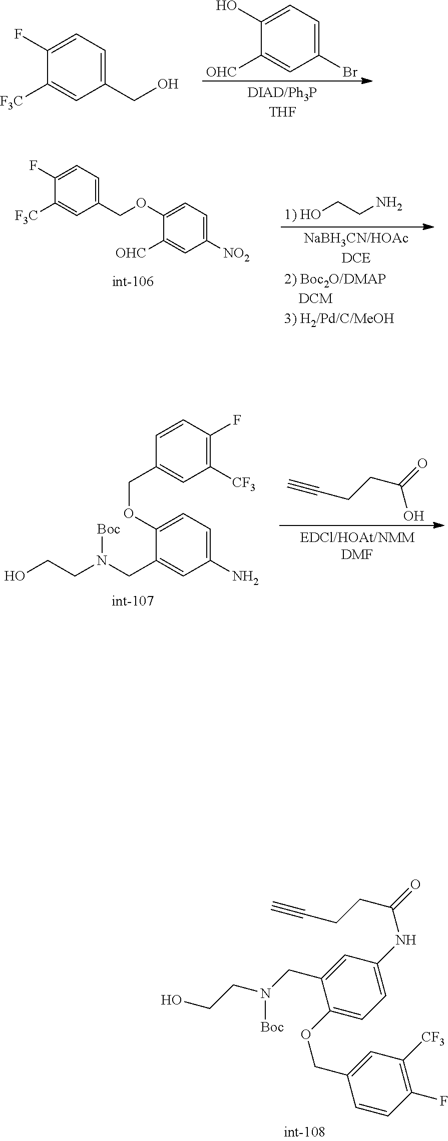

### Intermediate Synthesis 39 Synthesis of Intermediates Int-109 and Int-110

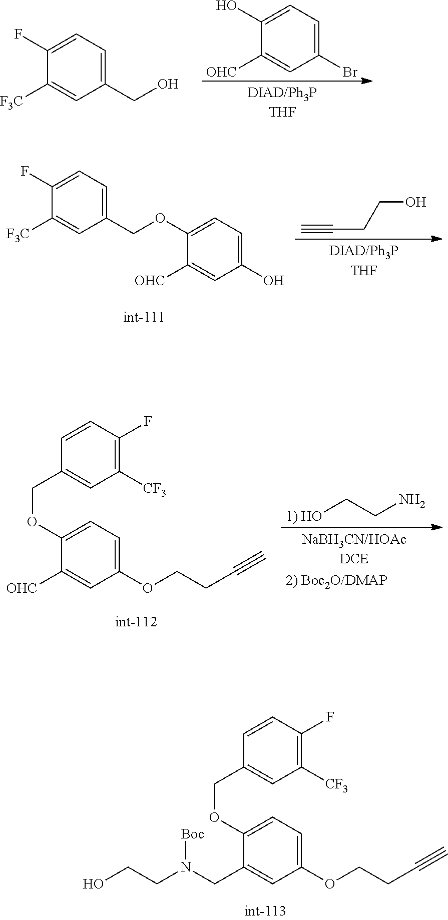

### Intermediate Synthesis 39 Synthesis of Intermediates Int-111, Int-112 and Int-113

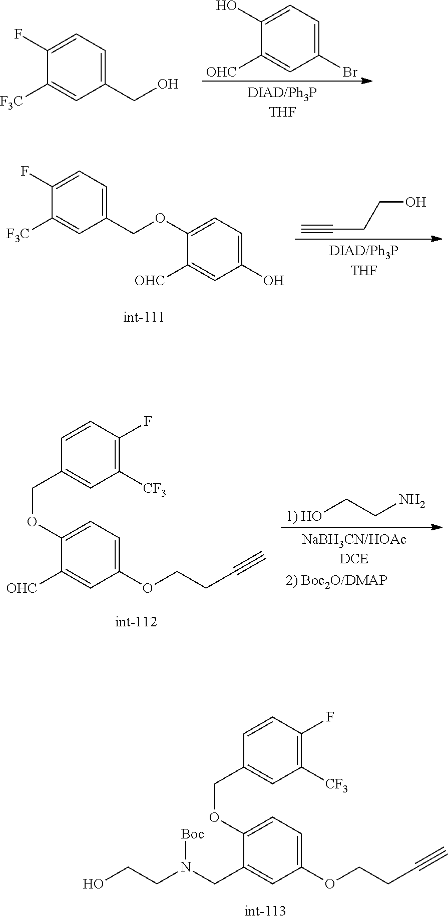

### Intermediate Synthesis 39 Synthesis of Intermediates Int-114 and Int-115

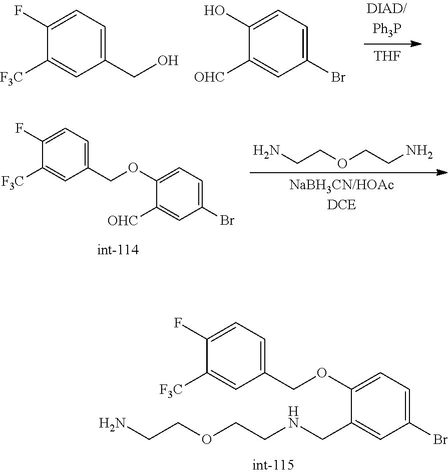 [SMILES: OCc1ccc(F)c(C(F)(F)F)c1 ; O=Cc1cc(Br)ccc1O ; O=Cc1cc(Br)ccc1OCc1ccc(F)c(C(F)(F)F)c1 ; NCCOCCN ; NCCOCCNCc1cc(Br)ccc1OCc1ccc(F)c(C(F)(F)F)c1 ; "THF"]

### Intermediate Synthesis 39 Synthesis of Intermediates Int-116 and Int-117

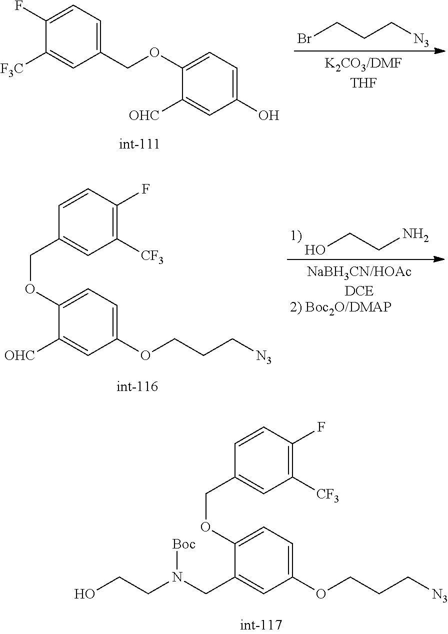

### Intermediate Synthesis 40 Synthesis of Intermediates Int-118 and Int-119

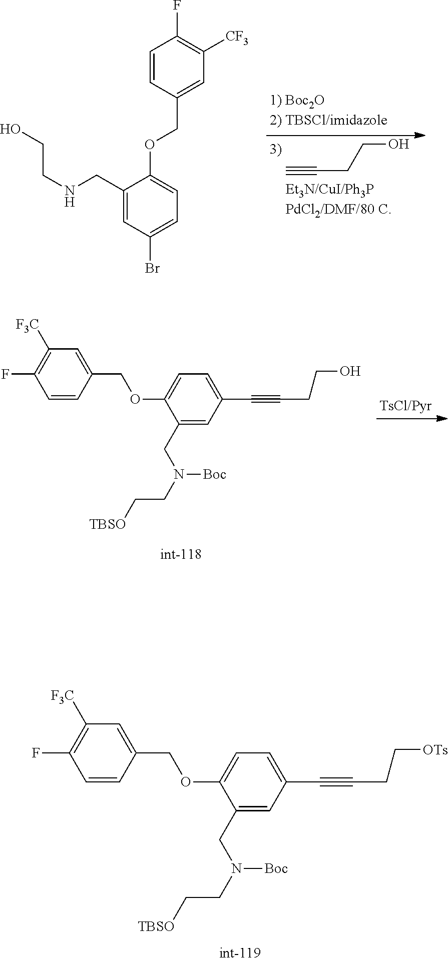

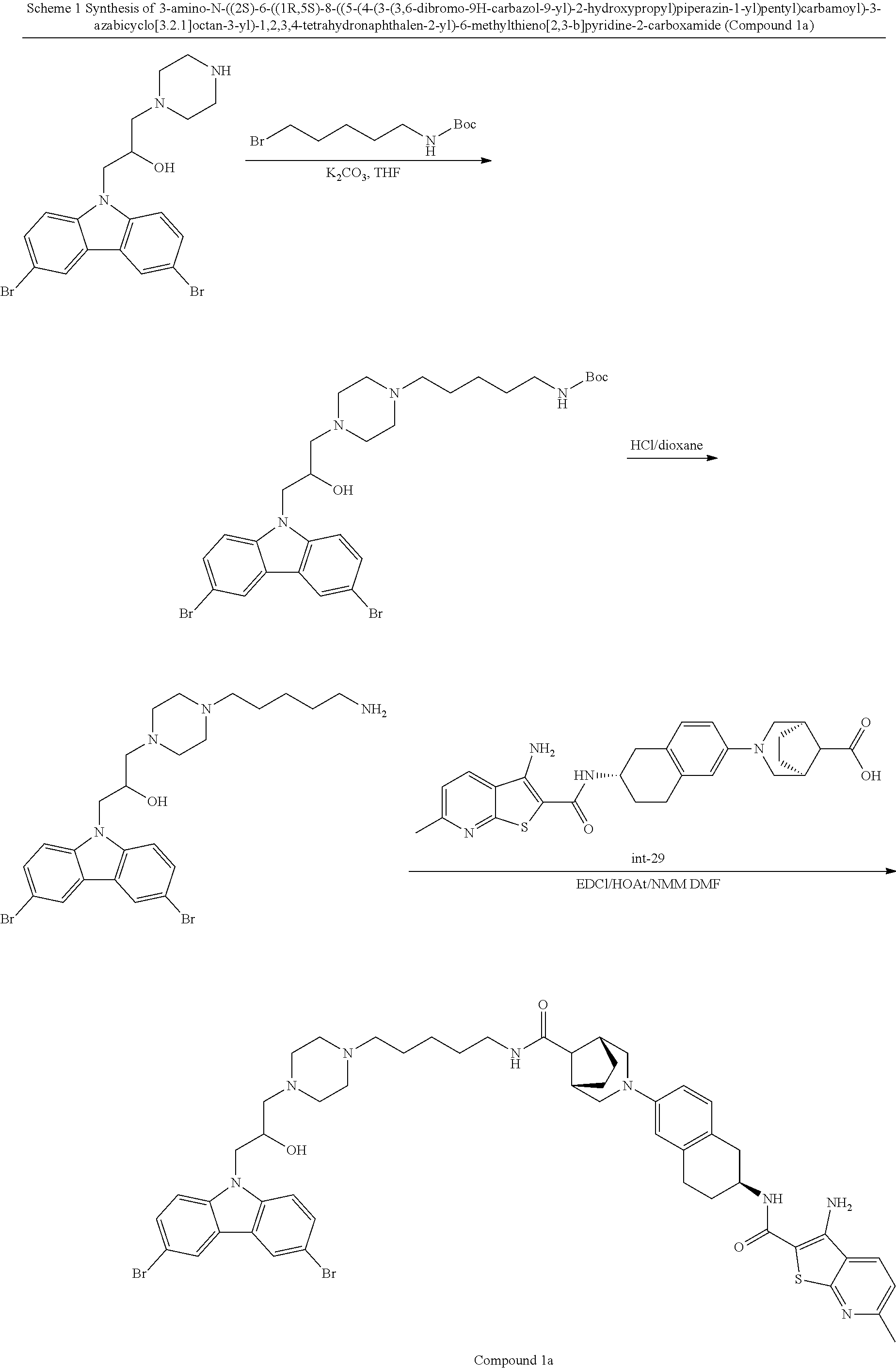 [SMILES: OC(CN1CCNCC1)Cn1c2ccc(Br)cc2c2cc(Br)ccc21 ; CC(C)(C)OC(=O)NCCCCCBr ; CC(C)(C)OC(=O)NCCCCCN1CCN(CC(O)Cn2c3ccc(Br)cc3c3cc(Br)ccc32)CC1 ; NCCCCCN1CCN(CC(O)Cn2c3ccc(Br)cc3c3cc(Br)ccc32)CC1 ; Cc1ccc2c(N)c(C(=O)N[C@H]3CCc4cc(N5C[C@H]6CC[C@@H](C5)C6C(=O)O)ccc4C3)sc2n1 ; Cc1ccc2c(N)c(C(=O)N[C@H]3CCc4cc(N5C[C@H]6CC[C@@H](C5)C6C(=O)NCCCCCN5CCN(CC(O)Cn6c7ccc(Br)cc7c7cc(Br)ccc76)CC5)ccc4C3)sc2n1]

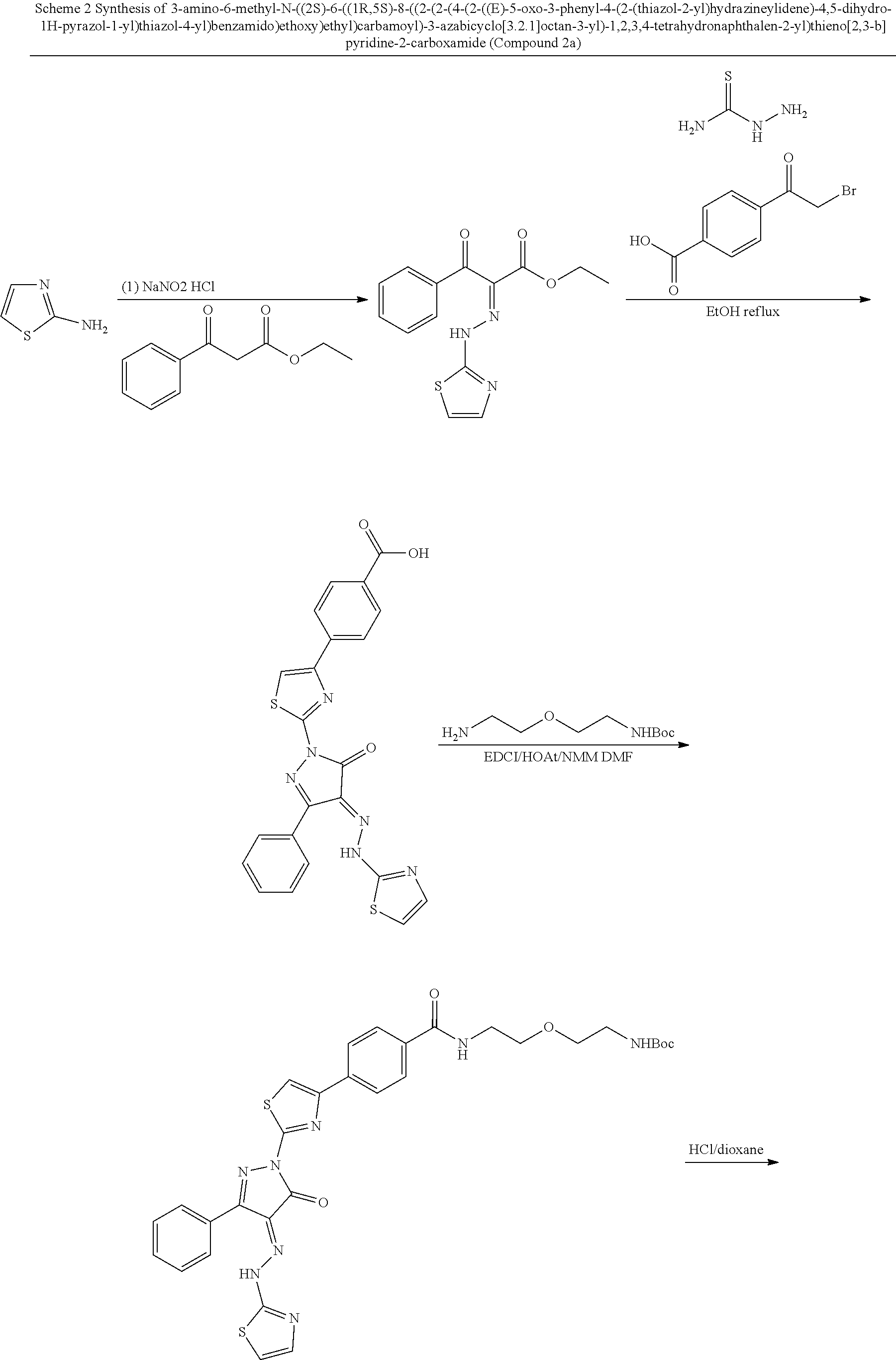

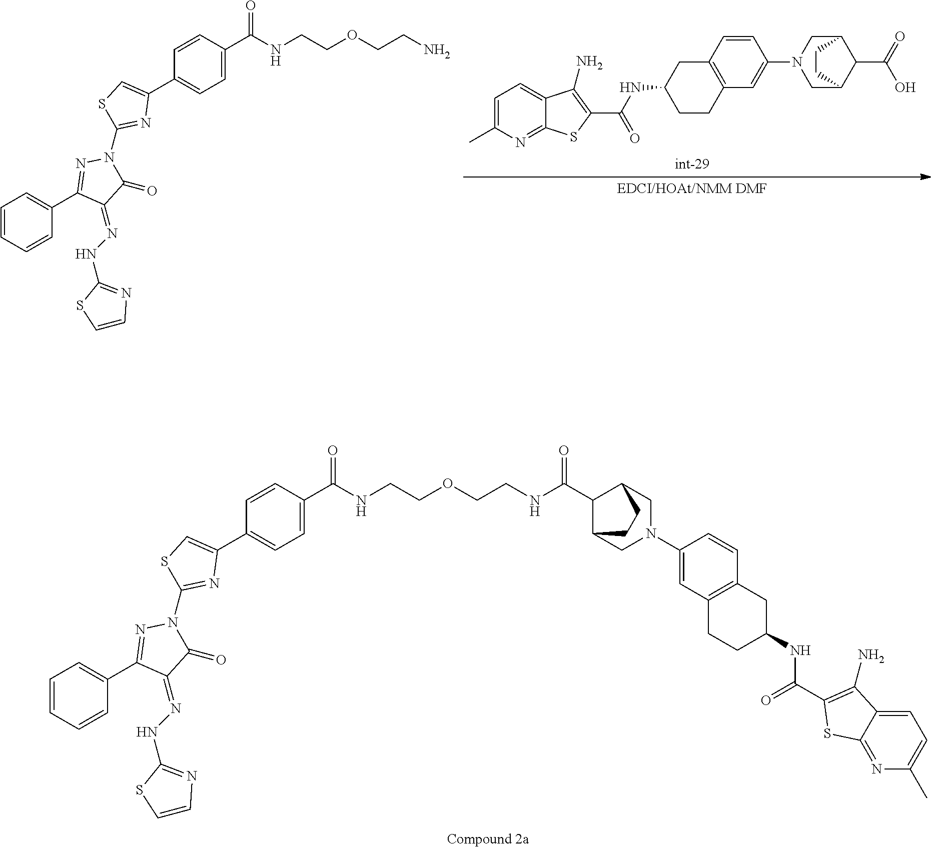 [SMILES: NCCOCCNC(=O)c1ccc(-c2csc(N3N=C(c4ccccc4)/C(=N\Nc4nccs4)C3=O)n2)cc1 ; Cc1ccc2c(N)c(C(=O)N[C@H]3CCc4cc(N5C[C@H]6CC[C@@H](C5)C6C(=O)O)ccc4C3)sc2n1 ; Cc1ccc2c(N)c(C(=O)N[C@H]3CCc4cc(N5C[C@H]6CC[C@@H](C5)C6C(=O)NCCOCCNC(=O)c5ccc(-c6csc(N7N=C(c8ccccc8)/C(=N\Nc8nccs8)C7=O)n6)cc5)ccc4C3)sc2n1]

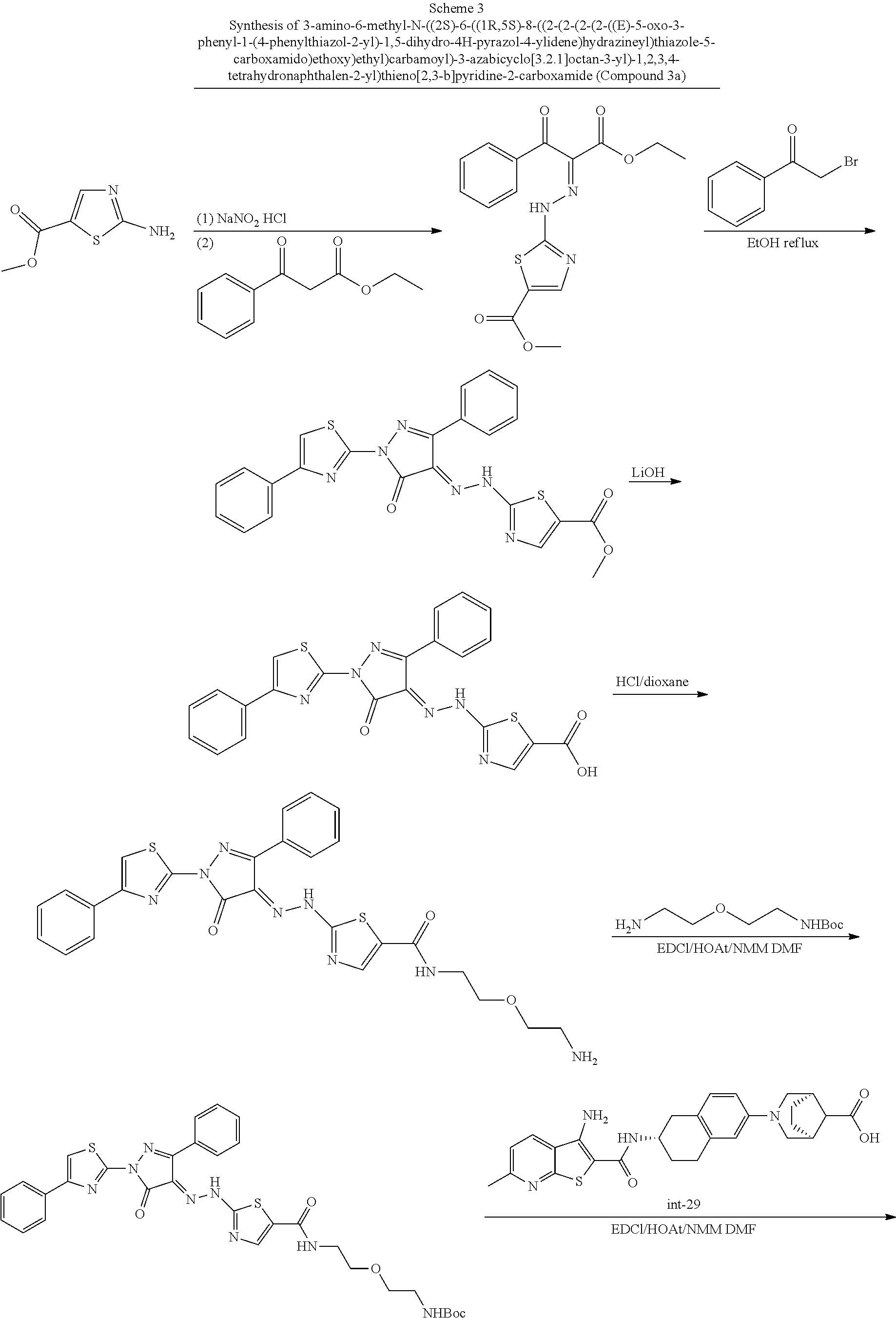

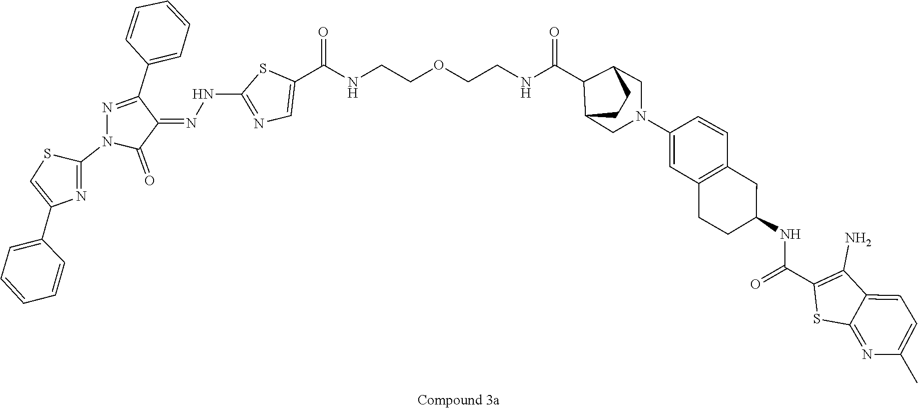 [SMILES: Cc1ccc2c(N)c(C(=O)N[C@H]3CCc4cc(N5C[C@H]6CC[C@@H](C5)C6C(=O)NCCOCCNC(=O)c5cnc(N/N=C6/C(=O)N(c7nc(-c8ccccc8)cs7)N=C6c6ccccc6)s5)ccc4C3)sc2n1]

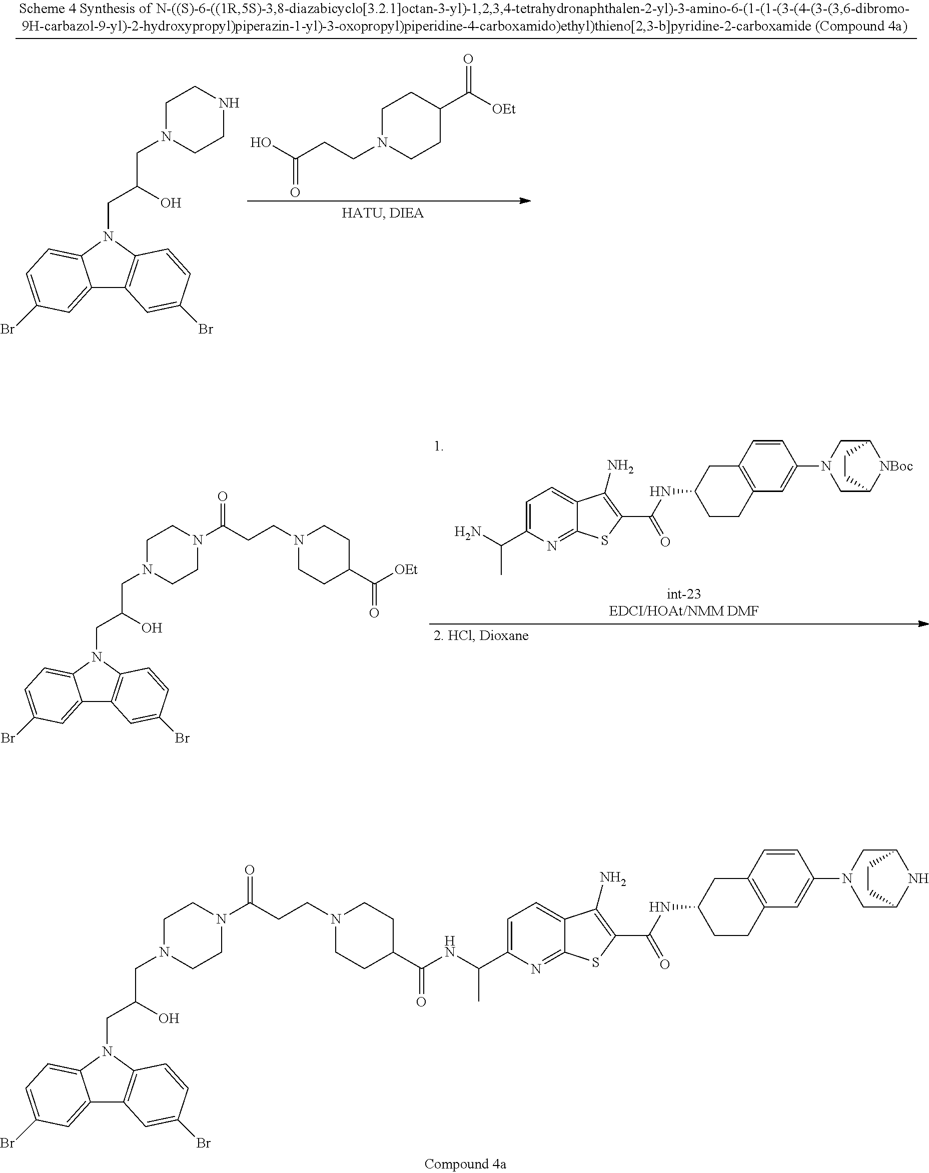

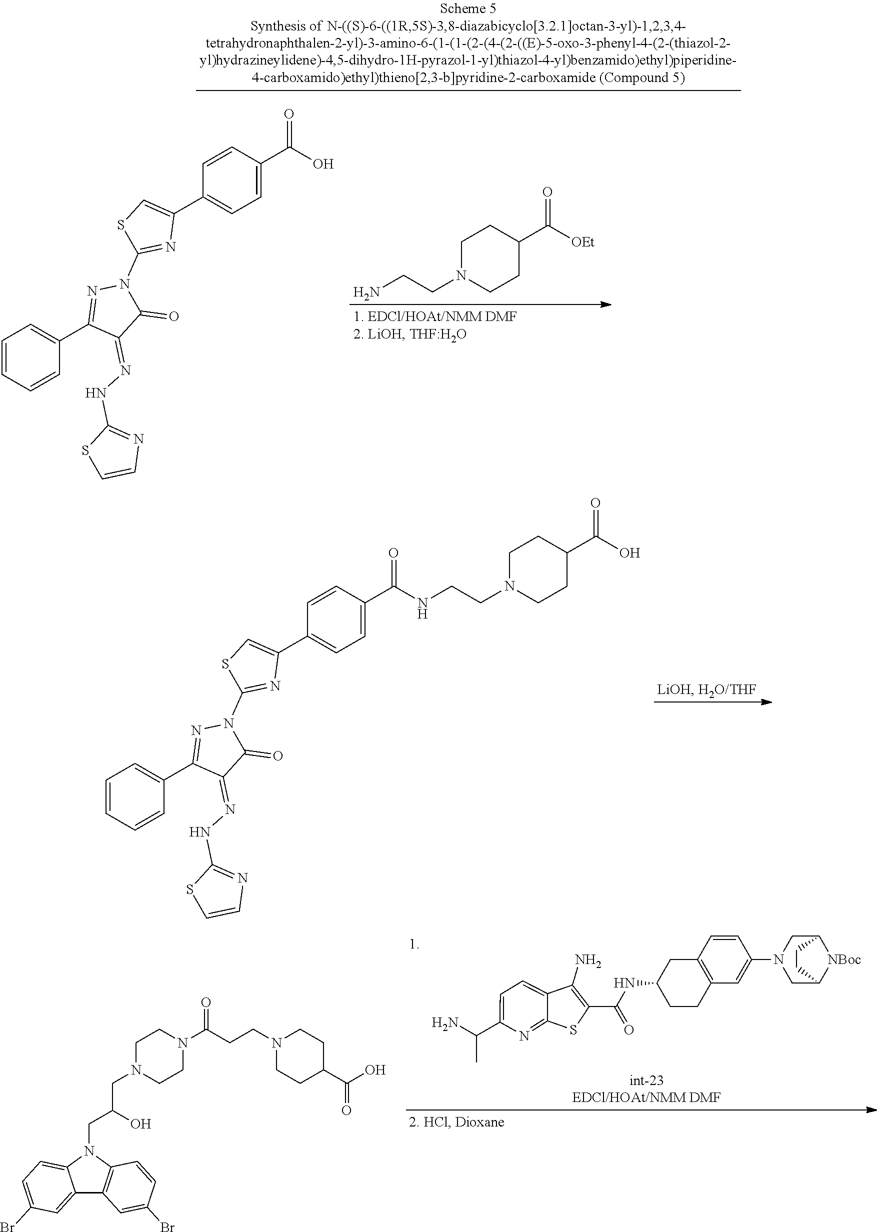 [SMILES: O=C(O)c1ccc(-c2csc(N3N=C(c4ccccc4)/C(=N\Nc4nccs4)C3=O)n2)cc1 ; CCOC(=O)C1CCN(CCN)CC1 ; O=C(NCCN1CCC(C(=O)O)CC1)c1ccc(-c2csc(N3N=C(c4ccccc4)/C(=N\Nc4nccs4)C3=O)n2)cc1 ; O=C(O)C1CCN(CCC(=O)N2CCN(CC(O)Cn3c4ccc(Br)cc4c4cc(Br)ccc43)CC2)CC1 ; CC(N)c1ccc2c(N)c(C(=O)N[C@H]3CCc4cc(N5C[C@H]6CC[C@@H](C5)N6C(=O)OC(C)(C)C)ccc4C3)sc2n1]

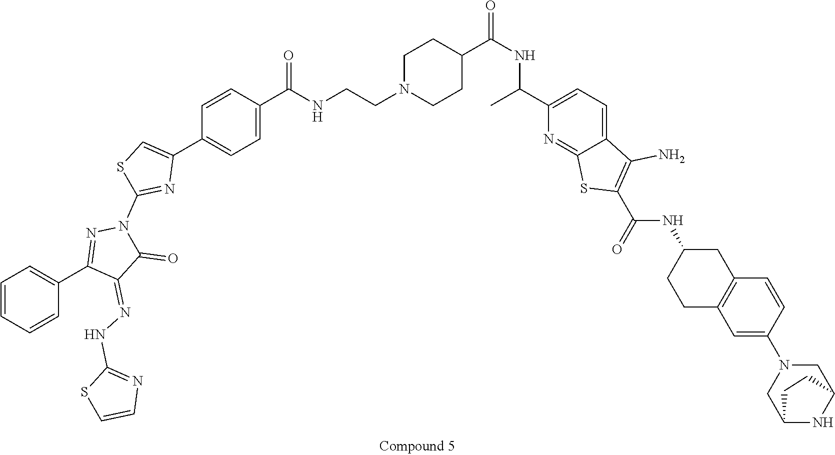 [SMILES: CC(NC(=O)C1CCN(CCNC(=O)c2ccc(-c3csc(N4N=C(c5ccccc5)/C(=N\Nc5nccs5)C4=O)n3)cc2)CC1)c1ccc2c(N)c(C(=O)N[C@H]3CCc4cc(N5C[C@H]6CC[C@@H](C5)N6)ccc4C3)sc2n1]

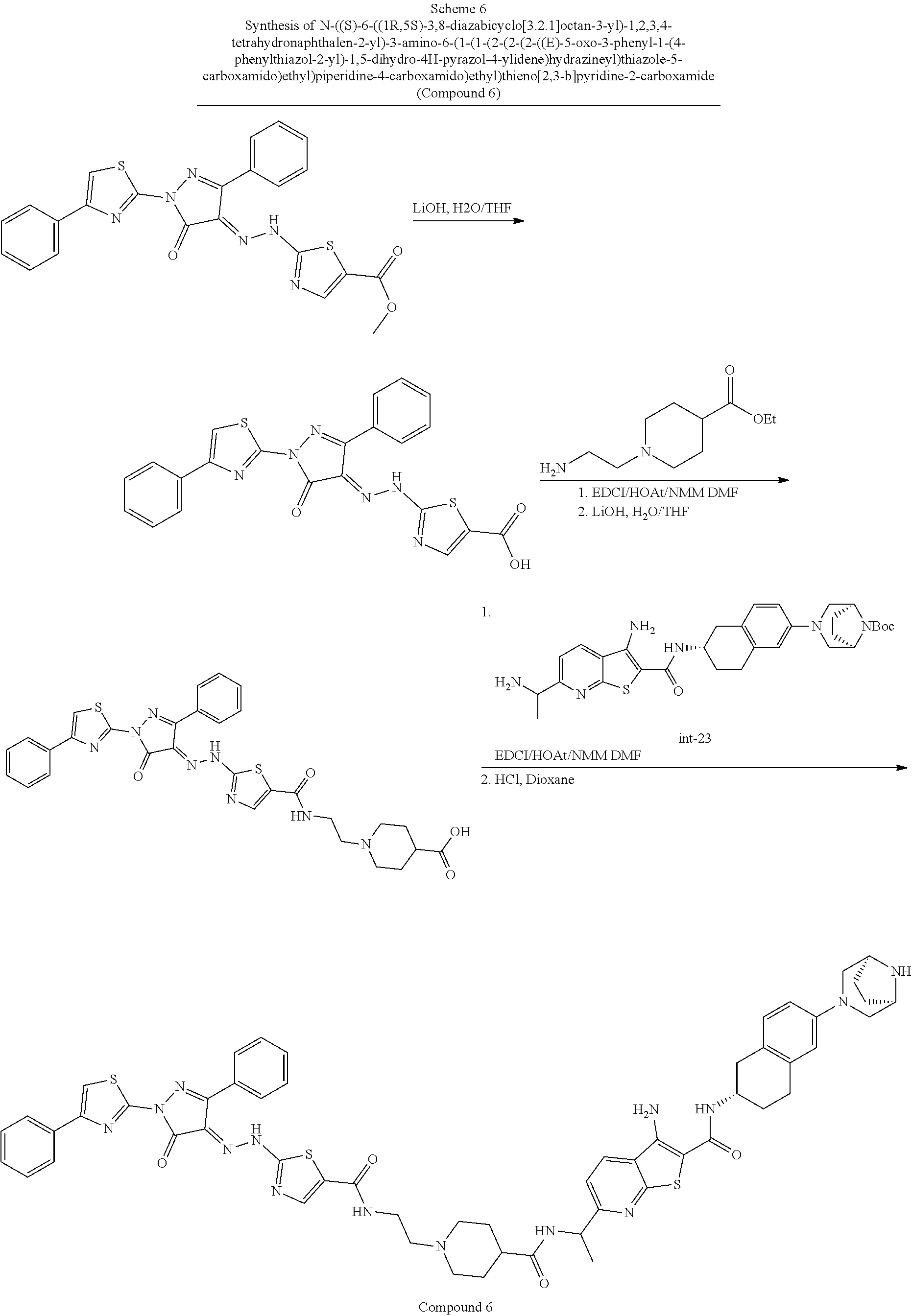

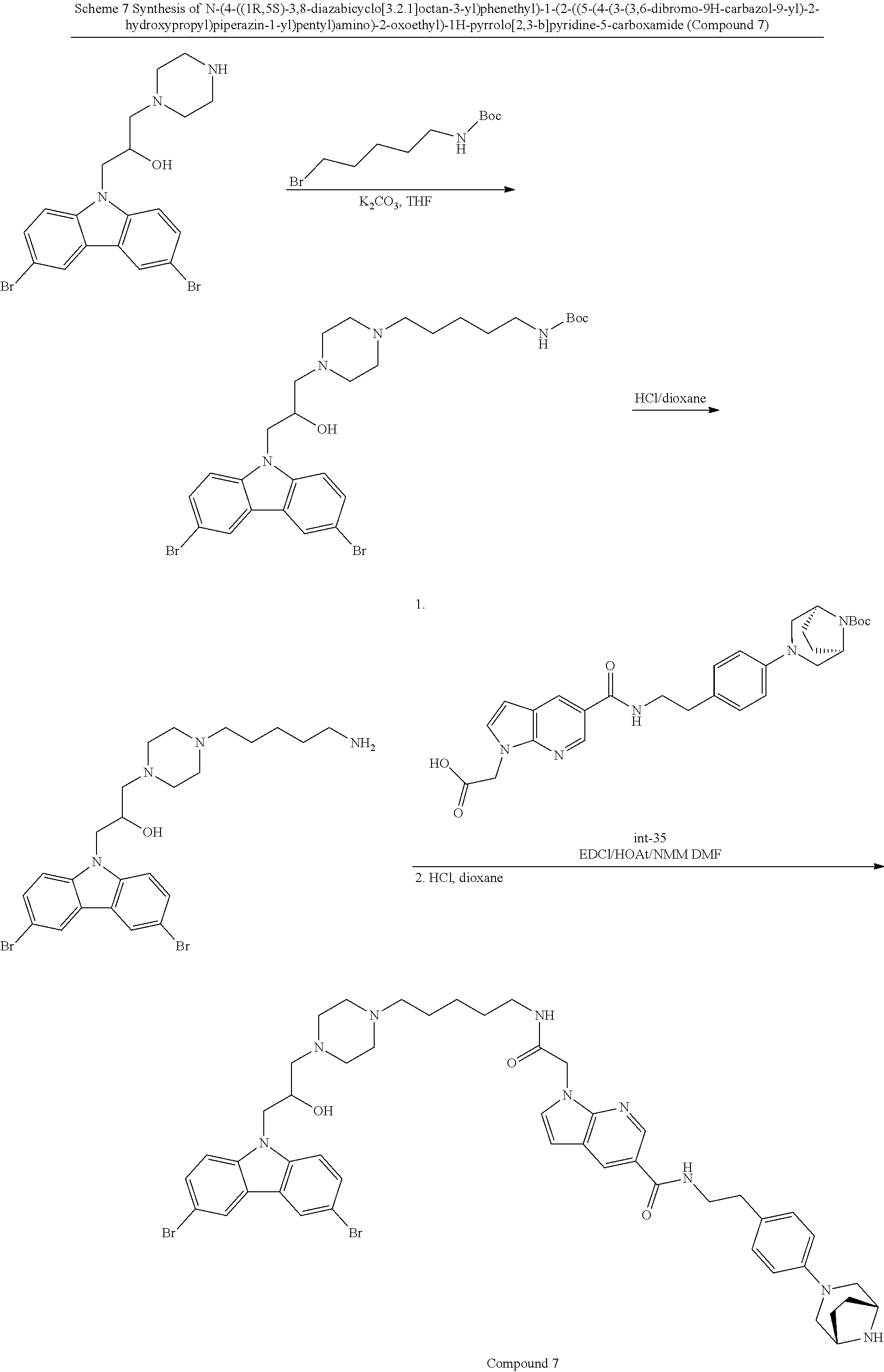 [SMILES: OC(CN1CCNCC1)Cn1c2ccc(Br)cc2c2cc(Br)ccc21 ; CC(C)(C)OC(=O)NCCCCCBr ; CC(C)(C)OC(=O)NCCCCCN1CCN(CC(O)Cn2c3ccc(Br)cc3c3cc(Br)ccc32)CC1 ; NCCCCCN1CCN(CC(O)Cn2c3ccc(Br)cc3c3cc(Br)ccc32)CC1 ; CC(C)(C)OC(=O)N1[C@@H]2CC[C@H]1CN(c1ccc(CCNC(=O)c3cnc4c(ccn4CC(=O)O)c3)cc1)C2 ; O=C(Cn1ccc2cc(C(=O)NCCc3ccc(N4C[C@H]5CC[C@@H](C4)N5)cc3)cnc21)NCCCCCN1CCN(CC(O)Cn2c3ccc(Br)cc3c3cc(Br)ccc32)CC1]

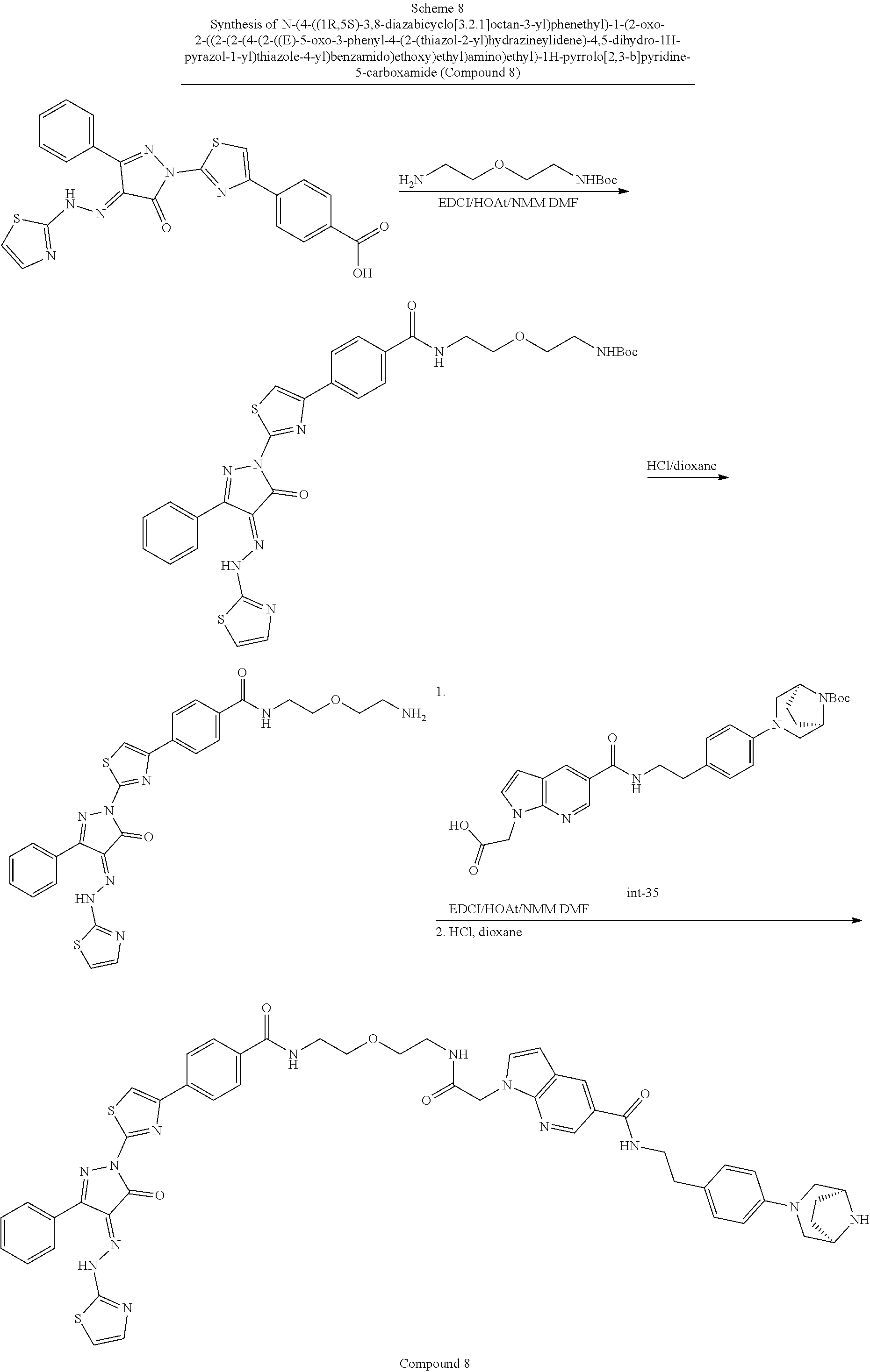

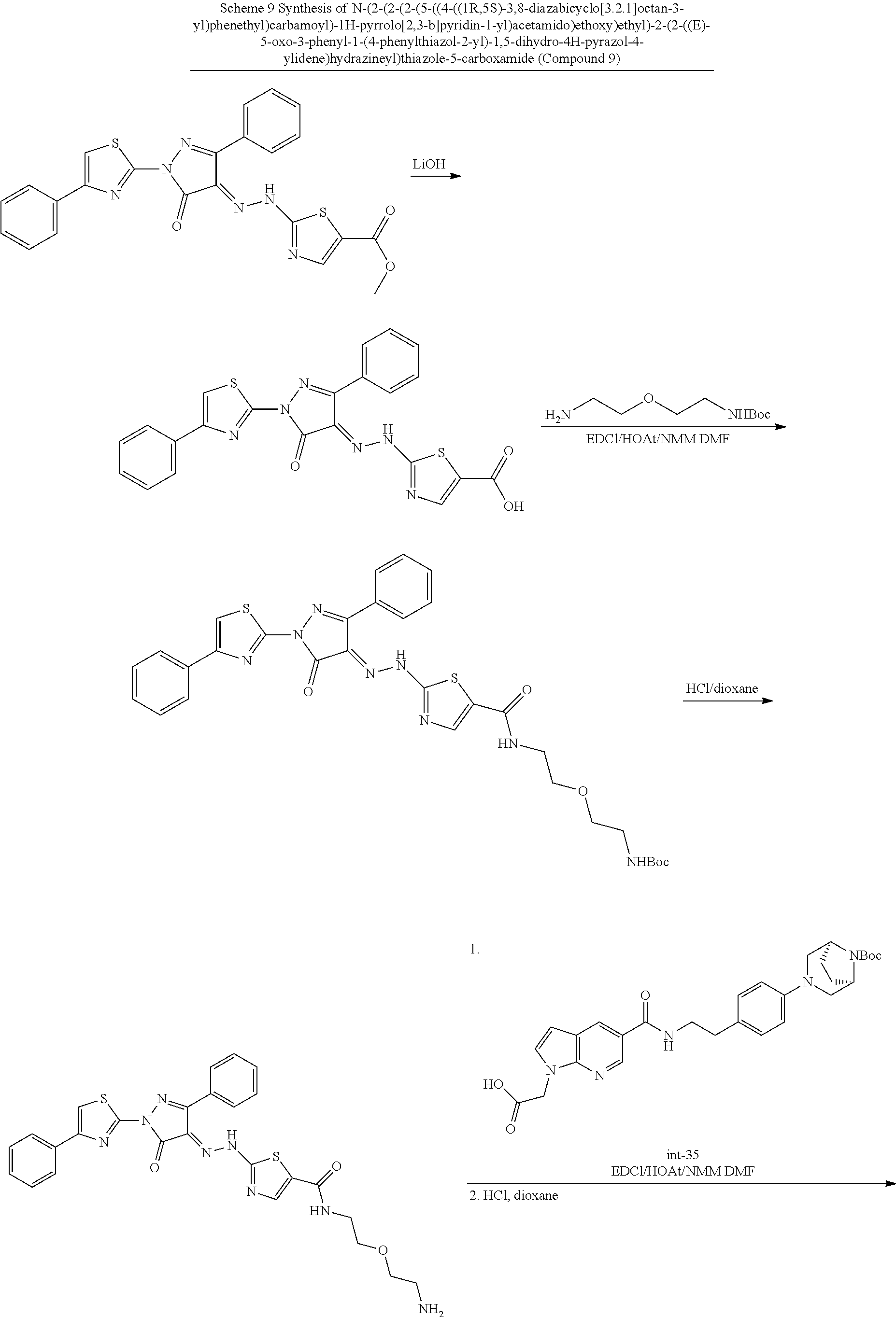

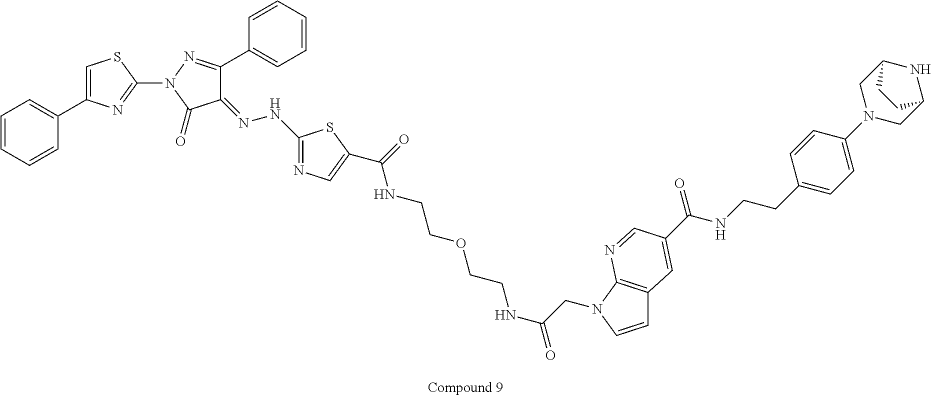 [SMILES: O=C(Cn1ccc2cc(C(=O)NCCc3ccc(N4C[C@H]5CC[C@@H](C4)N5)cc3)cnc21)NCCOCCNC(=O)c1cnc(N/N=C2/C(=O)N(c3nc(-c4ccccc4)cs3)N=C2c2ccccc2)s1]

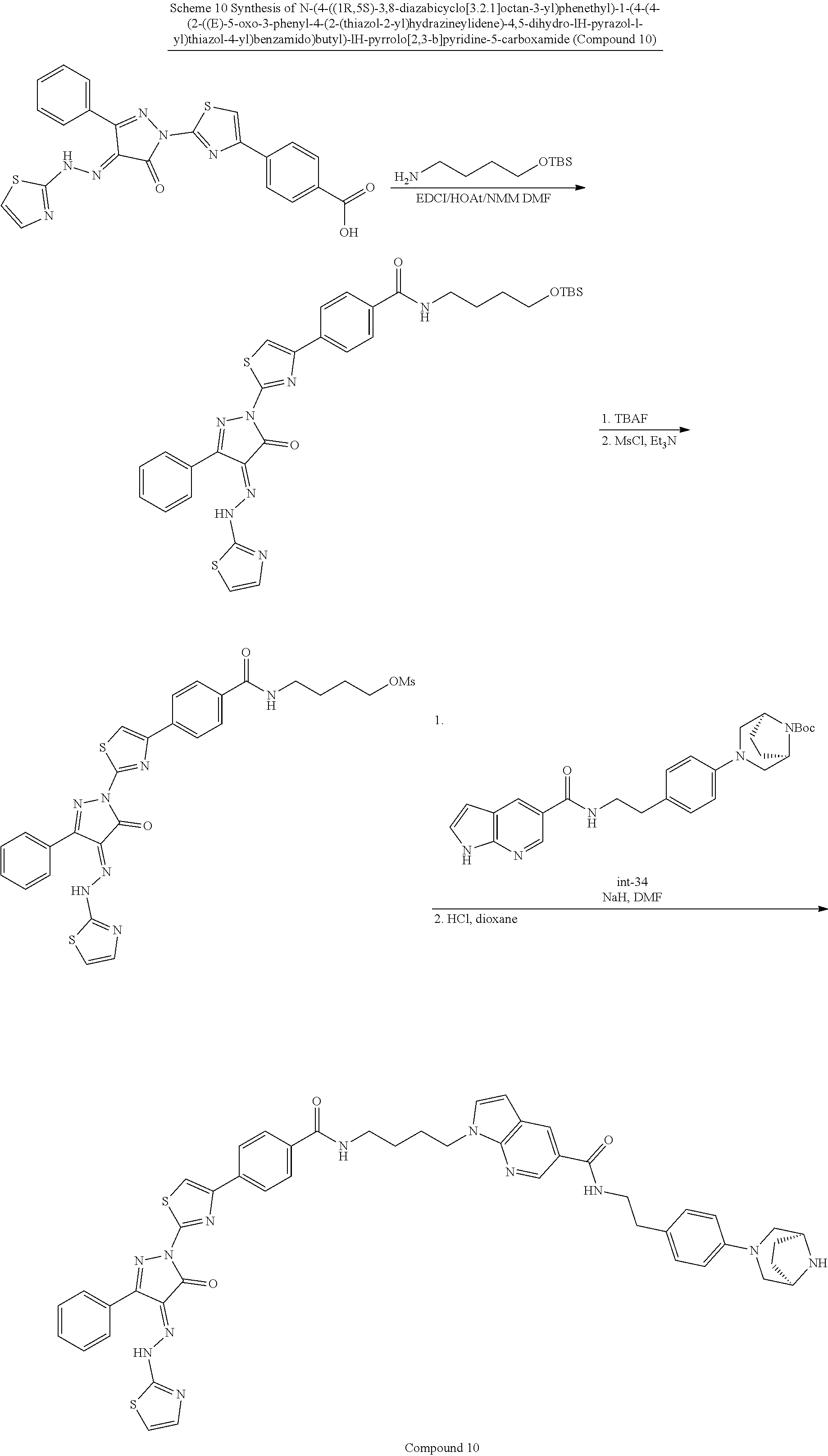 [SMILES: O=C(O)c1ccc(-c2csc(N3N=C(c4ccccc4)/C(=N\Nc4nccs4)C3=O)n2)cc1 ; CC(C)(C)[Si](C)(C)OCCCCN ; CC(C)(C)[Si](C)(C)OCCCCNC(=O)c1ccc(-c2csc(N3N=C(c4ccccc4)/C(=N\Nc4nccs4)C3=O)n2)cc1 ; CS(=O)(=O)OCCCCNC(=O)c1ccc(-c2csc(N3N=C(c4ccccc4)/C(=N\Nc4nccs4)C3=O)n2)cc1 ; CC(C)(C)OC(=O)N1[C@@H]2CC[C@H]1CN(c1ccc(CCNC(=O)c3cnc4[nH]ccc4c3)cc1)C2 ; O=C(NCCCCn1ccc2cc(C(=O)NCCc3ccc(N4C[C@H]5CC[C@@H](C4)N5)cc3)cnc21)c1ccc(-c2csc(N3N=C(c4ccccc4)/C(=N\Nc4nccs4)C3=O)n2)cc1]

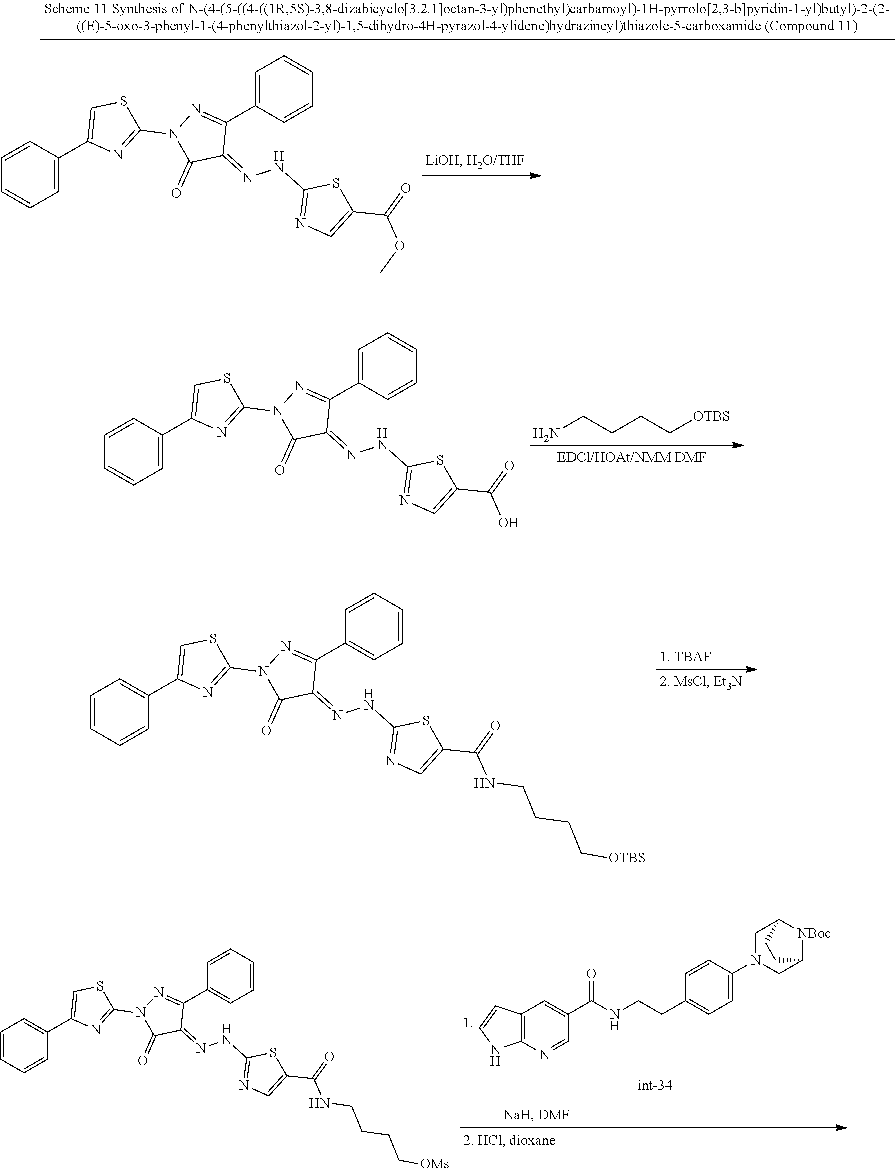 [SMILES: COC(=O)c1cnc(N/N=C2/C(=O)N(c3nc(-c4ccccc4)cs3)N=C2c2ccccc2)s1 ; O=C(O)c1cnc(N/N=C2/C(=O)N(c3nc(-c4ccccc4)cs3)N=C2c2ccccc2)s1 ; CC(C)(C)[Si](C)(C)OCCCCN ; CC(C)(C)[Si](C)(C)OCCCCNC(=O)c1cnc(N/N=C2/C(=O)N(c3nc(-c4ccccc4)cs3)N=C2c2ccccc2)s1 ; CS(=O)(=O)OCCCCNC(=O)c1cnc(N/N=C2/C(=O)N(c3nc(-c4ccccc4)cs3)N=C2c2ccccc2)s1 ; CC(C)(C)OC(=O)N1[C@@H]2CC[C@H]1CN(c1ccc(CCNC(=O)c3cnc4[nH]ccc4c3)cc1)C2]

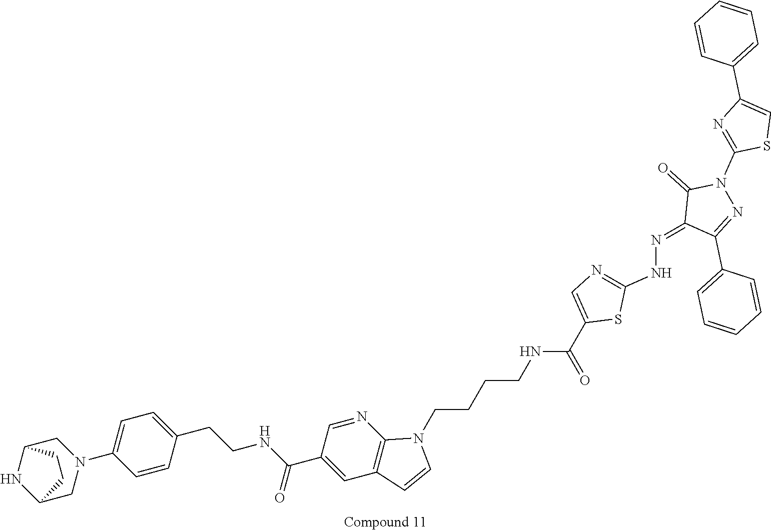 [SMILES: O=C(NCCc1ccc(N2C[C@H]3CC[C@@H](C2)N3)cc1)c1cnc2c(ccn2CCCCNC(=O)c2cnc(N/N=C3/C(=O)N(c4nc(-c5ccccc5)cs4)N=C3c3ccccc3)s2)c1]

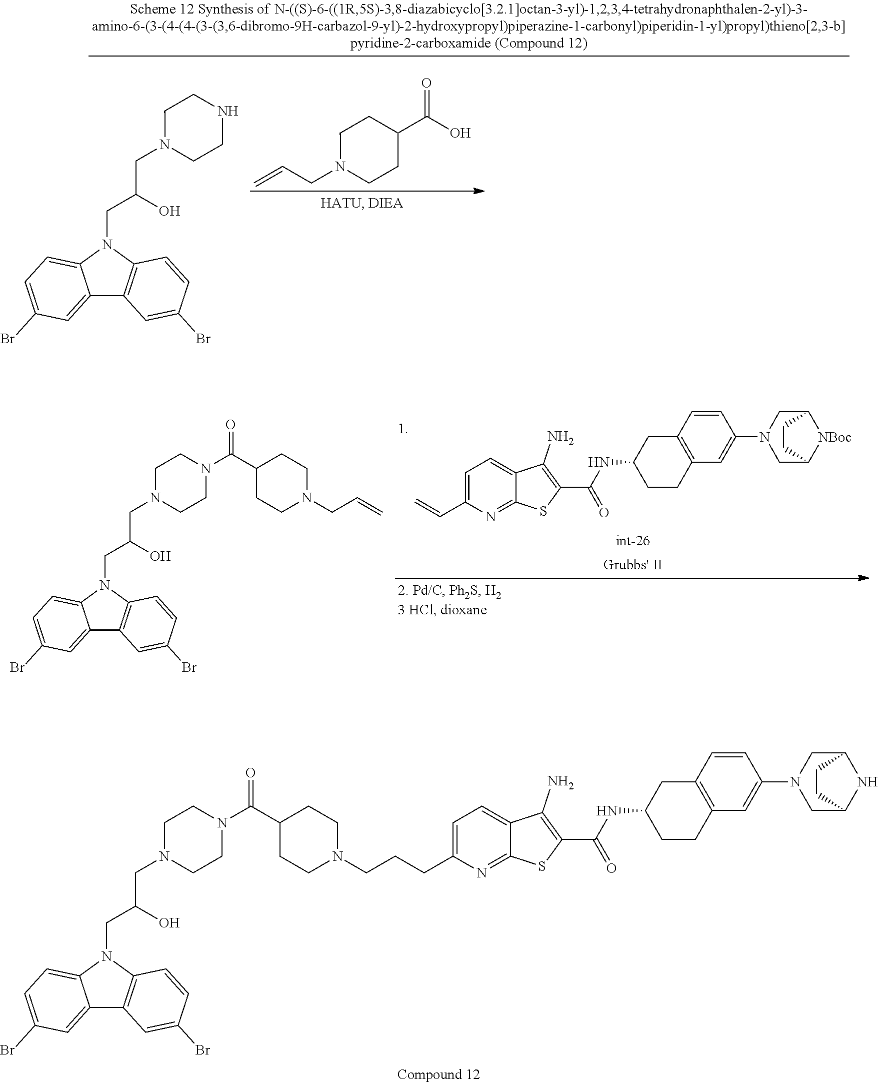

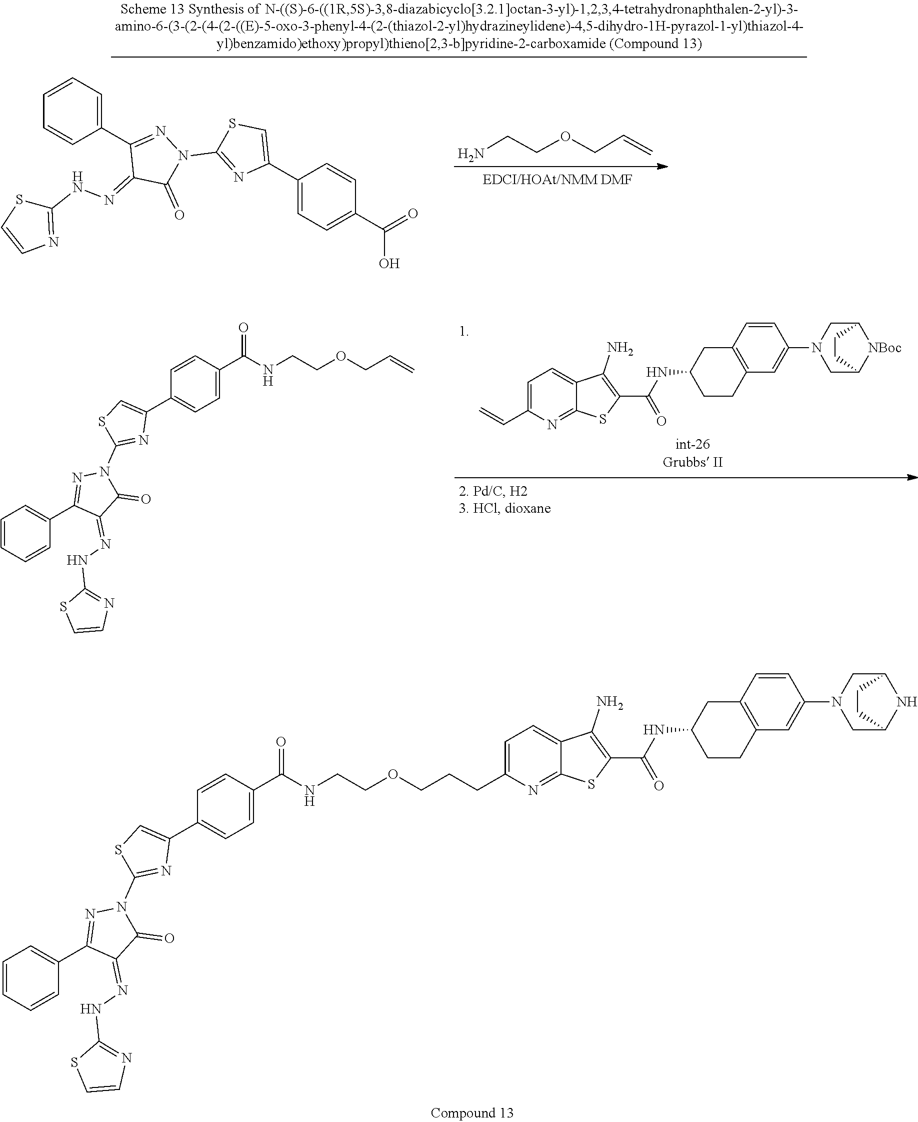

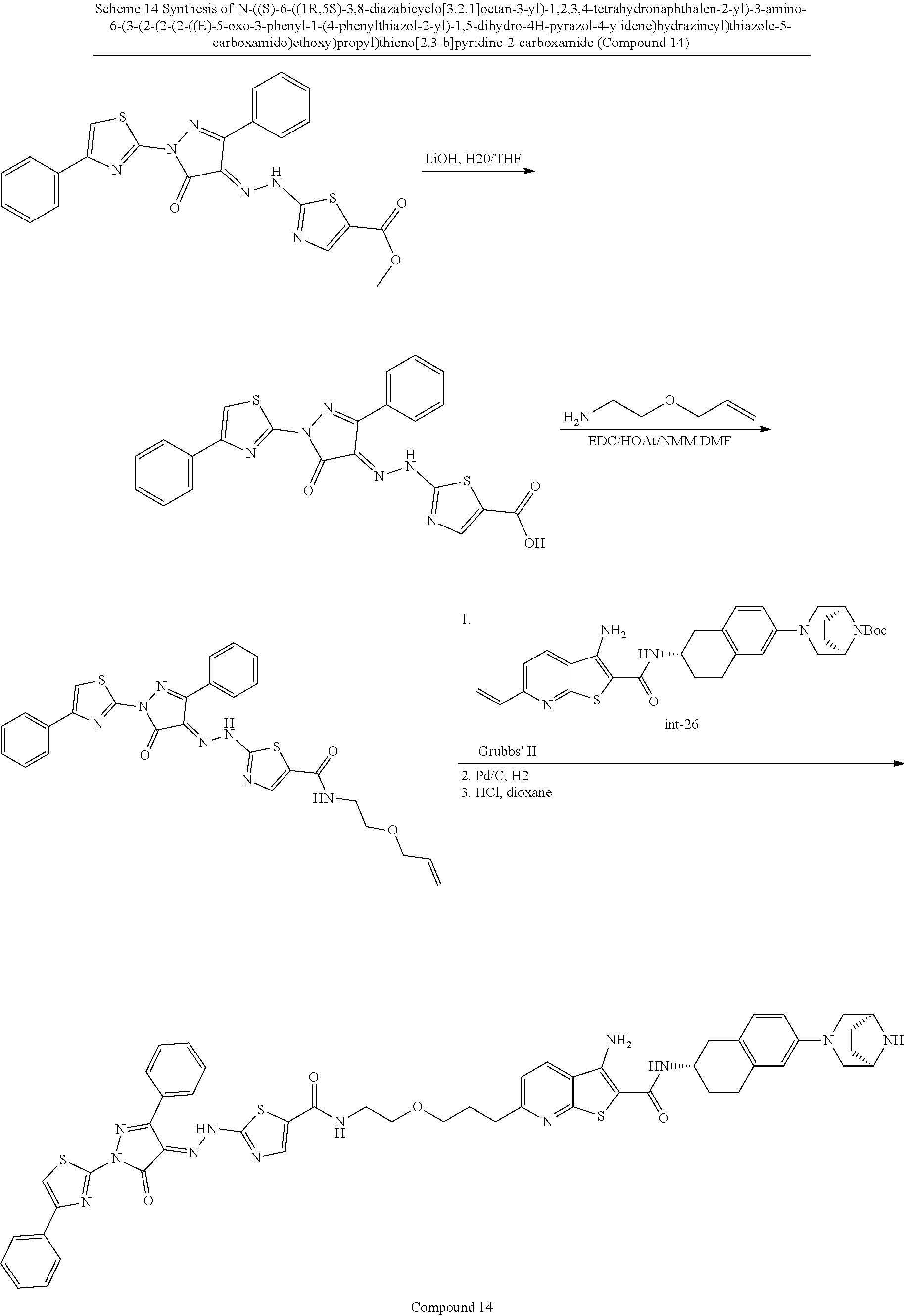

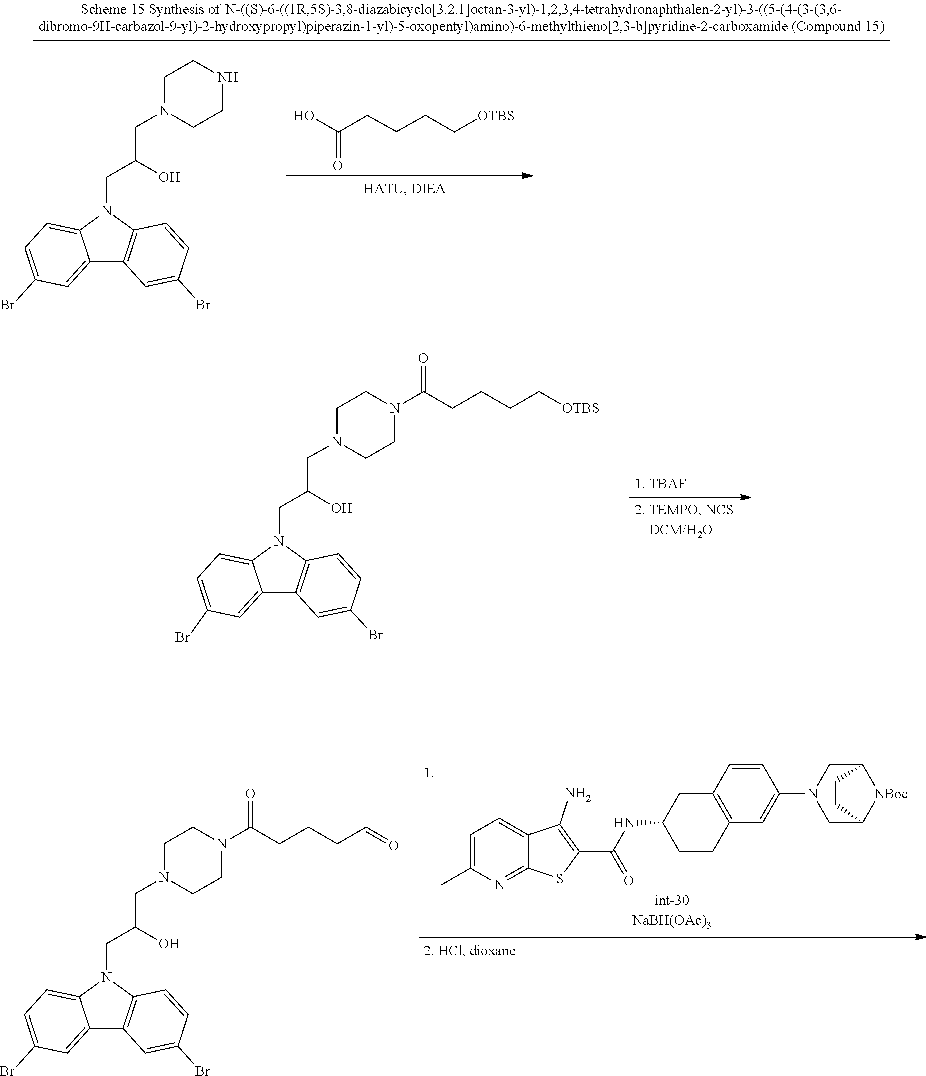 [SMILES: OC(CN1CCNCC1)Cn1c2ccc(Br)cc2c2cc(Br)ccc21 ; CC(C)(C)[Si](C)(C)OCCCCC(=O)O ; CC(C)(C)[Si](C)(C)OCCCCC(=O)N1CCN(CC(O)Cn2c3ccc(Br)cc3c3cc(Br)ccc32)CC1 ; O=CCCCC(=O)N1CCN(CC(O)Cn2c3ccc(Br)cc3c3cc(Br)ccc32)CC1 ; Cc1ccc2c(N)c(C(=O)N[C@H]3CCc4cc(N5C[C@H]6CC[C@@H](C5)N6C(=O)OC(C)(C)C)ccc4C3)sc2n1]

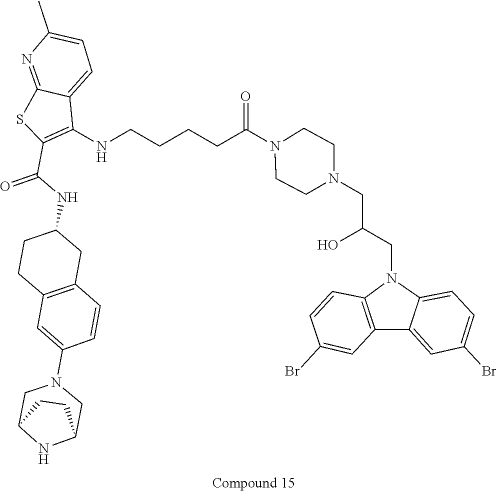 [SMILES: Cc1ccc2c(NCCCCC(=O)N3CCN(CC(O)Cn4c5ccc(Br)cc5c5cc(Br)ccc54)CC3)c(C(=O)N[C@H]3CCc4cc(N5C[C@H]6CC[C@@H](C5)N6)ccc4C3)sc2n1]

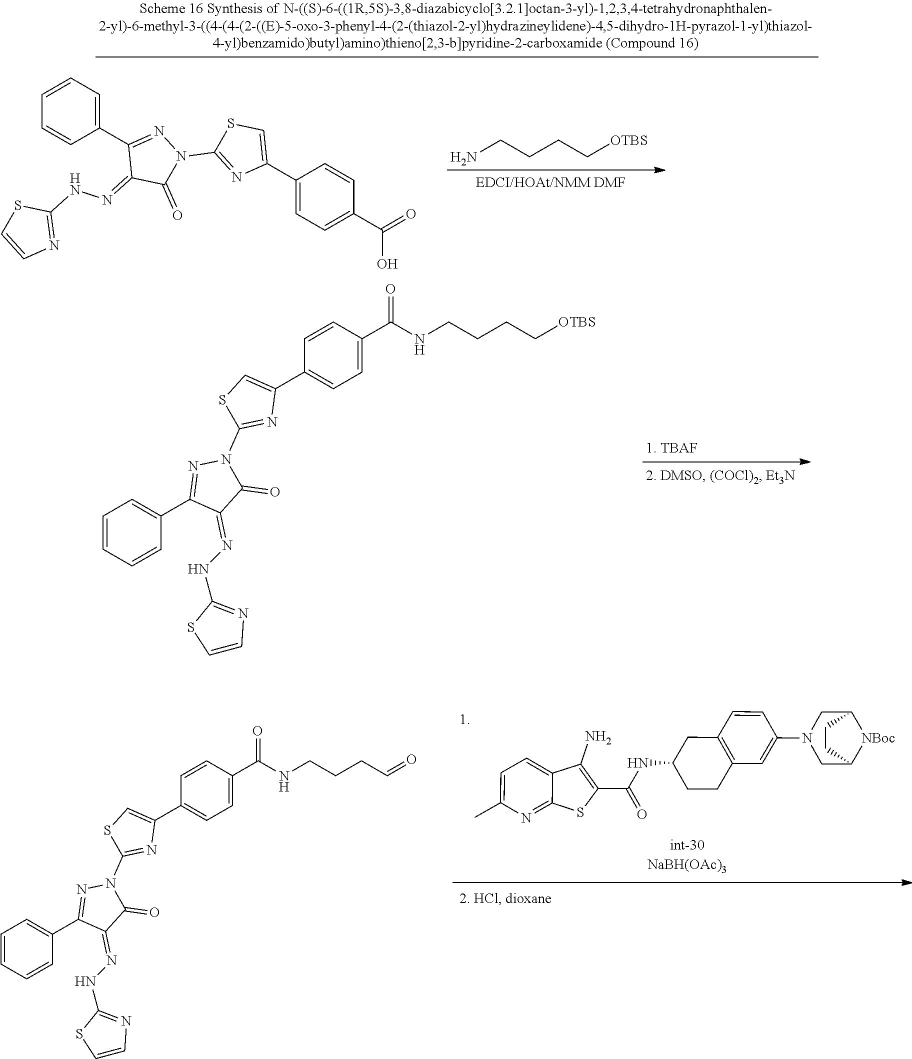

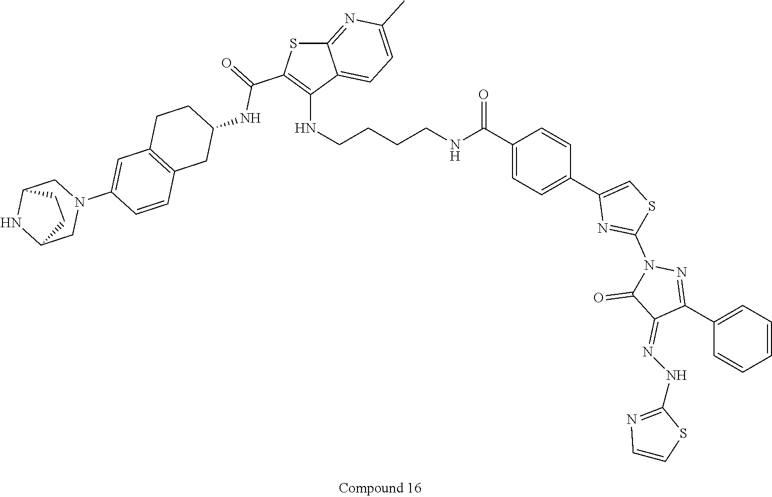 [SMILES: Cc1ccc2c(NCCCCNC(=O)c3ccc(-c4csc(N5N=C(c6ccccc6)/C(=N\Nc6nccs6)C5=O)n4)cc3)c(C(=O)N[C@H]3CCc4cc(N5C[C@H]6CC[C@@H](C5)N6)ccc4C3)sc2n1]

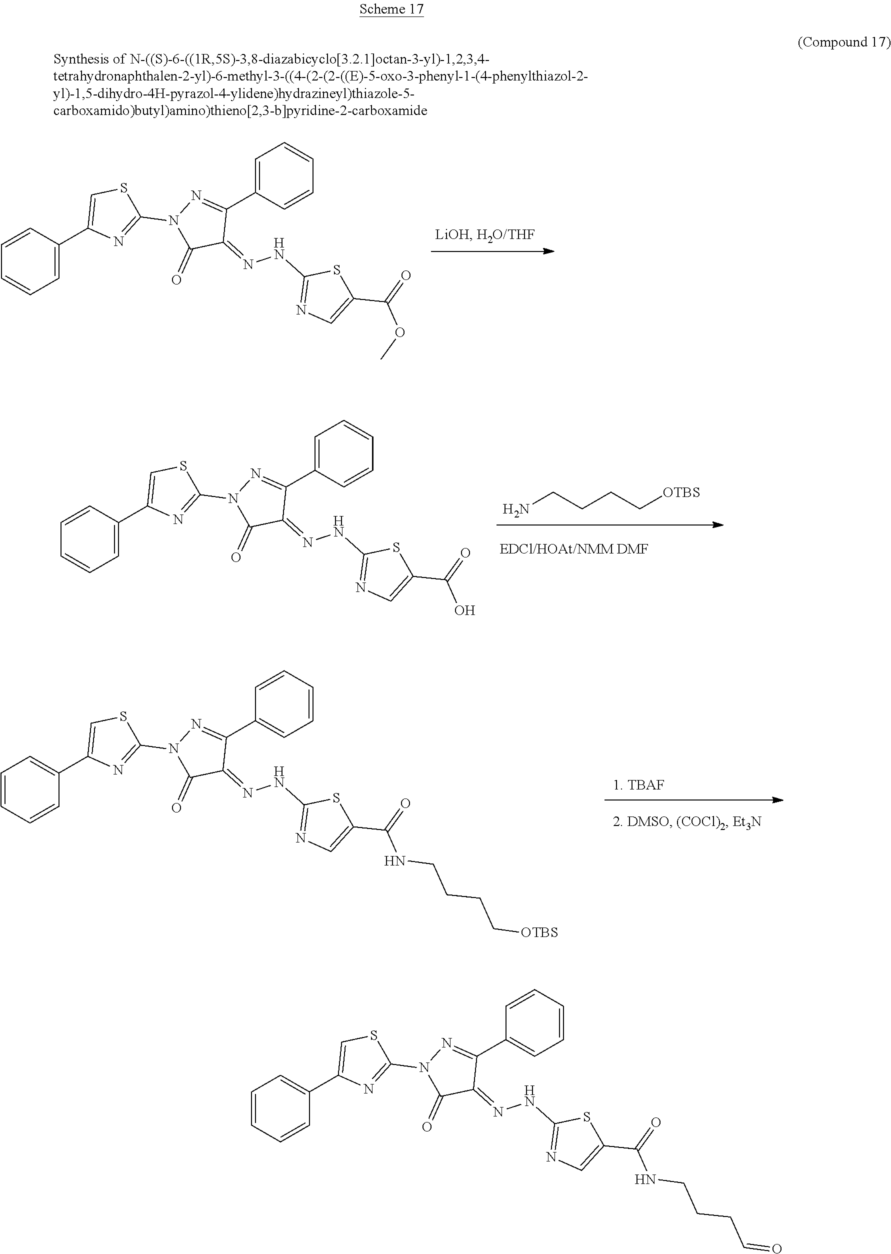 [SMILES: COC(=O)c1cnc(N/N=C2/C(=O)N(c3nc(-c4ccccc4)cs3)N=C2c2ccccc2)s1 ; O=C(O)c1cnc(N/N=C2/C(=O)N(c3nc(-c4ccccc4)cs3)N=C2c2ccccc2)s1 ; CC(C)(C)[Si](C)(C)OCCCCN ; CC(C)(C)[Si](C)(C)OCCCCNC(=O)c1cnc(N/N=C2/C(=O)N(c3nc(-c4ccccc4)cs3)N=C2c2ccccc2)s1 ; O=CCCCNC(=O)c1cnc(N/N=C2/C(=O)N(c3nc(-c4ccccc4)cs3)N=C2c2ccccc2)s1]

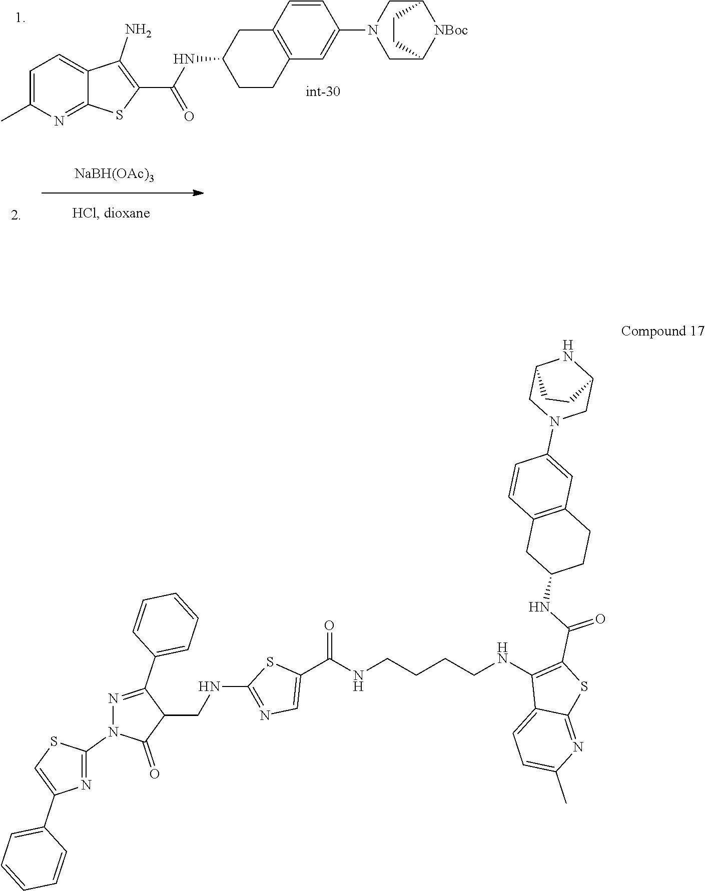 [SMILES: Cc1ccc2c(N)c(C(=O)N[C@H]3CCc4cc(N5C[C@H]6CC[C@@H](C5)N6C(=O)OC(C)(C)C)ccc4C3)sc2n1 ; Cc1ccc2c(NCCCCNC(=O)c3cnc(NCC4C(=O)N(c5nc(-c6ccccc6)cs5)N=C4c4ccccc4)s3)c(C(=O)N[C@H]3CCc4cc(N5C[C@H]6CC[C@@H](C5)N6)ccc4C3)sc2n1 ; "NaBH(OAc)3"]

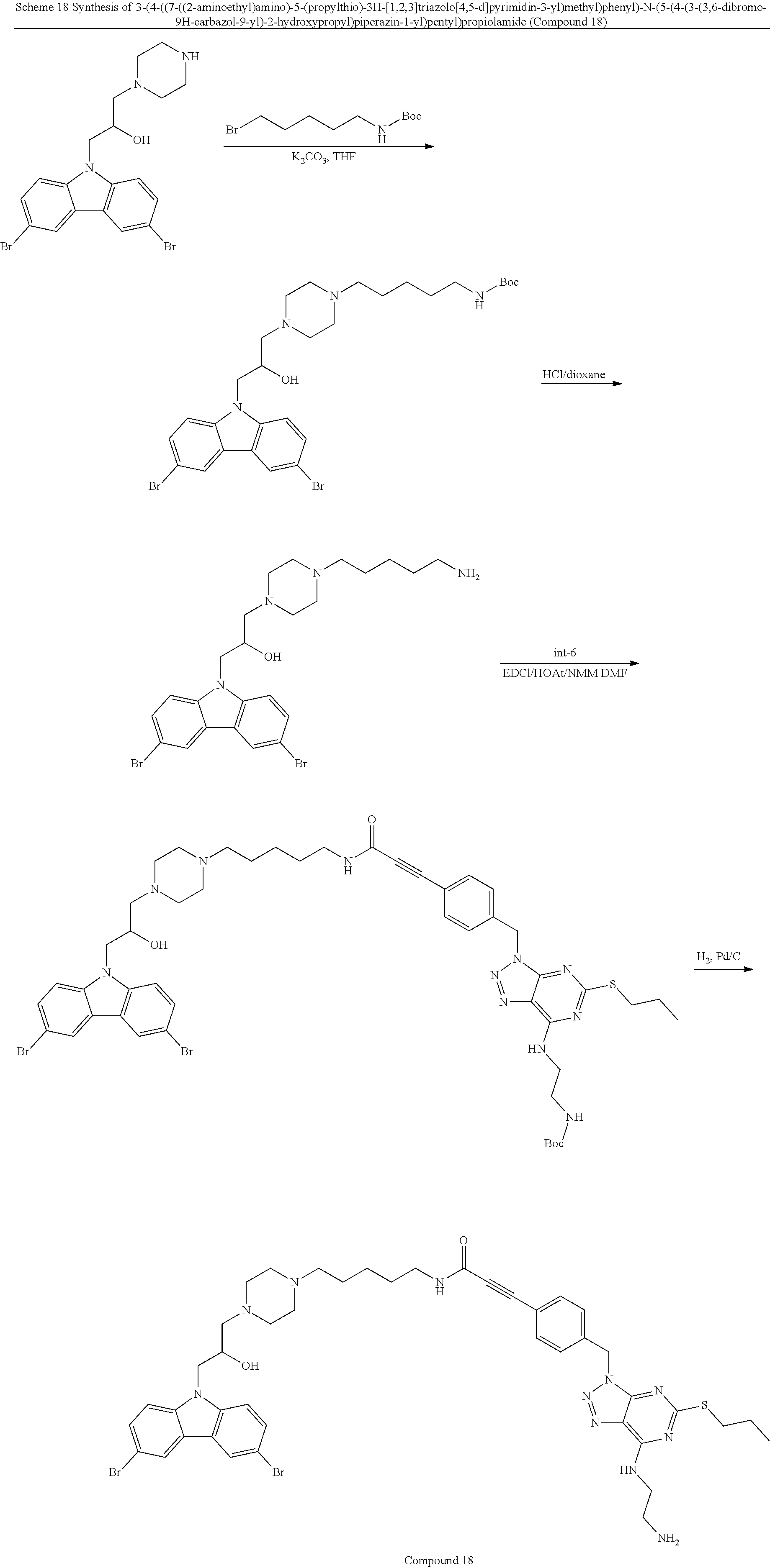 [SMILES: OC(CN1CCNCC1)Cn1c2ccc(Br)cc2c2cc(Br)ccc21 ; CC(C)(C)OC(=O)NCCCCCBr ; CC(C)(C)OC(=O)NCCCCCN1CCN(CC(O)Cn2c3ccc(Br)cc3c3cc(Br)ccc32)CC1 ; NCCCCCN1CCN(CC(O)Cn2c3ccc(Br)cc3c3cc(Br)ccc32)CC1 ; CCCSc1nc(NCCNC(=O)OC(C)(C)C)c2nnn(Cc3ccc(C#CC(=O)NCCCCCN4CCN(CC(O)Cn5c6ccc(Br)cc6c6cc(Br)ccc65)CC4)cc3)c2n1 ; CCCSc1nc(NCCN)c2nnn(Cc3ccc(C#CC(=O)NCCCCCN4CCN(CC(O)Cn5c6ccc(Br)cc6c6cc(Br)ccc65)CC4)cc3)c2n1]

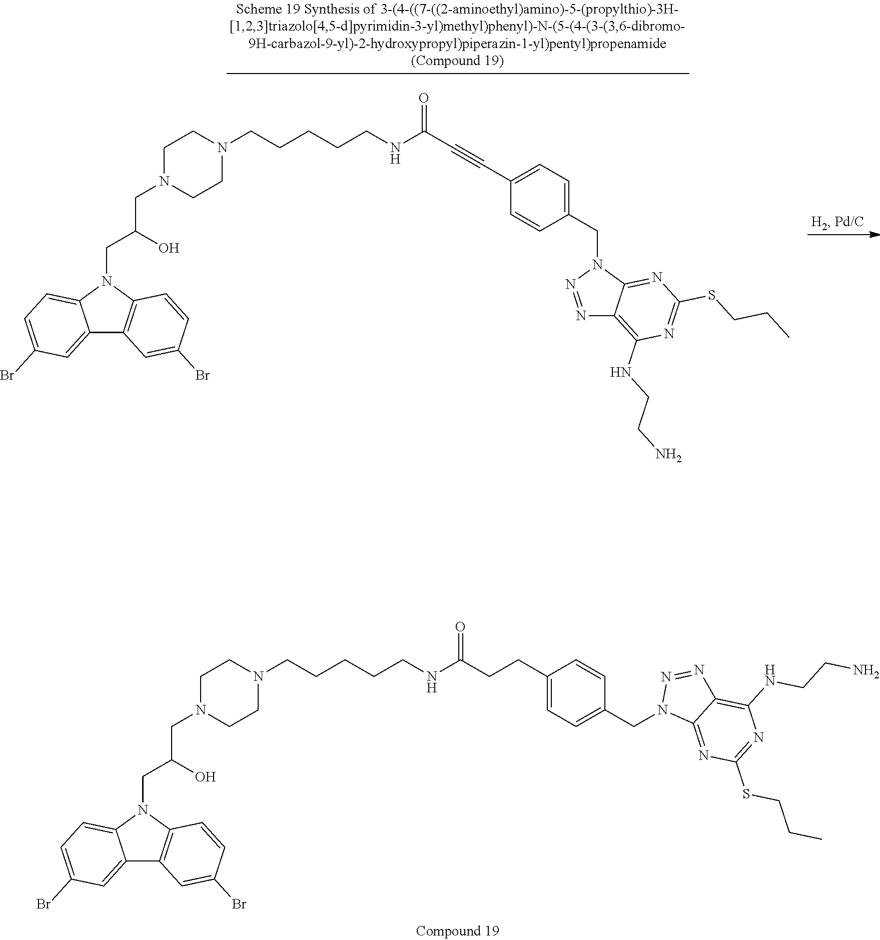 [SMILES: CCCSc1nc(NCCN)c2nnn(Cc3ccc(C#CC(=O)NCCCCCN4CCN(CC(O)Cn5c6ccc(Br)cc6c6cc(Br)ccc65)CC4)cc3)c2n1 ; CCCSc1nc(NCCN)c2nnn(Cc3ccc(CCC(=O)NCCCCCN4CCN(CC(O)Cn5c6ccc(Br)cc6c6cc(Br)ccc65)CC4)cc3)c2n1]

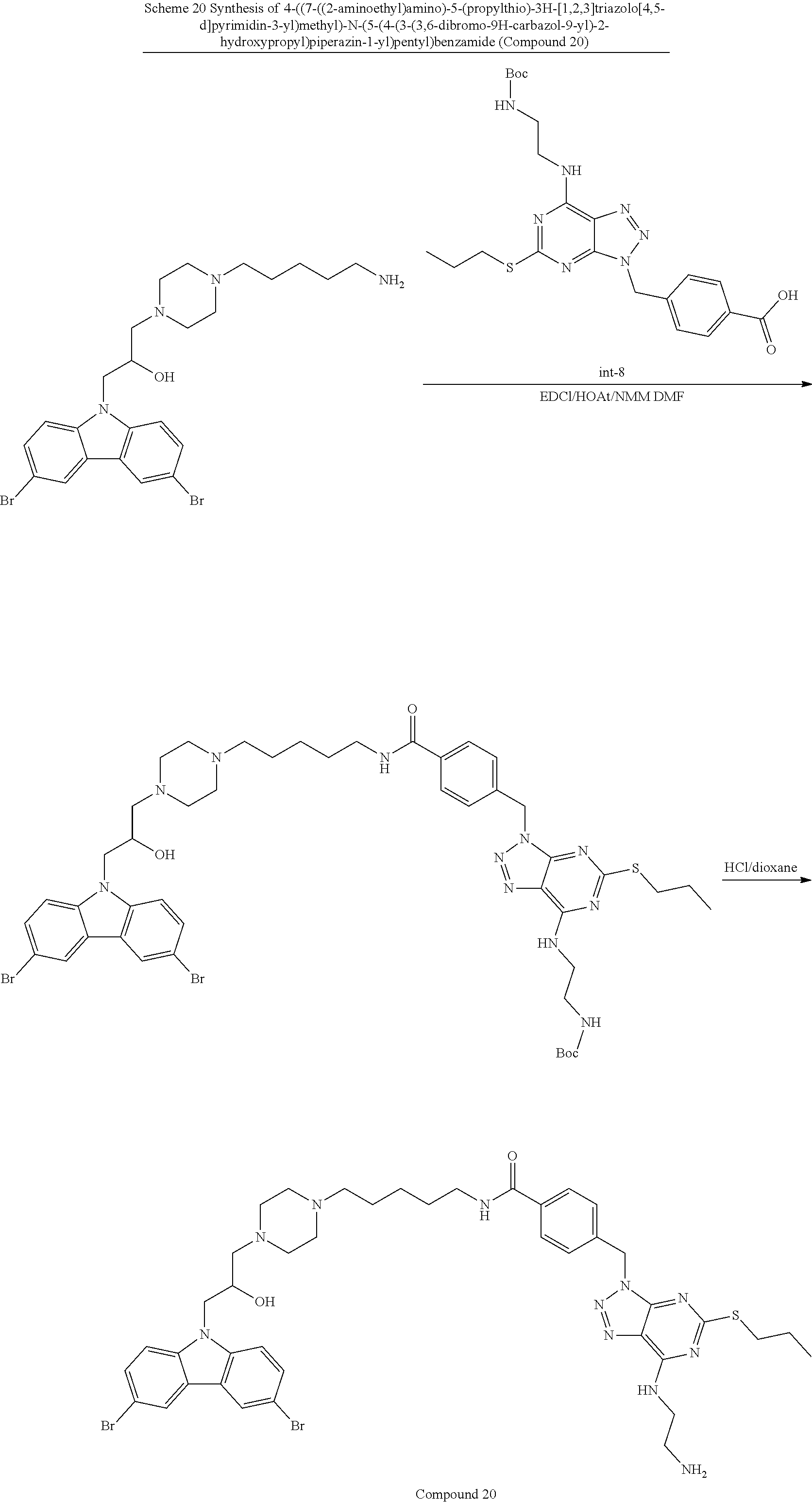 [SMILES: NCCCCCN1CCN(CC(O)Cn2c3ccc(Br)cc3c3cc(Br)ccc32)CC1 ; CCCSc1nc(NCCNC(=O)OC(C)(C)C)c2nnn(Cc3ccc(C(=O)O)cc3)c2n1 ; CCCSc1nc(NCCNC(=O)OC(C)(C)C)c2nnn(Cc3ccc(C(=O)NCCCCCN4CCN(CC(O)Cn5c6ccc(Br)cc6c6cc(Br)ccc65)CC4)cc3)c2n1 ; CCCSc1nc(NCCN)c2nnn(Cc3ccc(C(=O)NCCCCCN4CCN(CC(O)Cn5c6ccc(Br)cc6c6cc(Br)ccc65)CC4)cc3)c2n1]

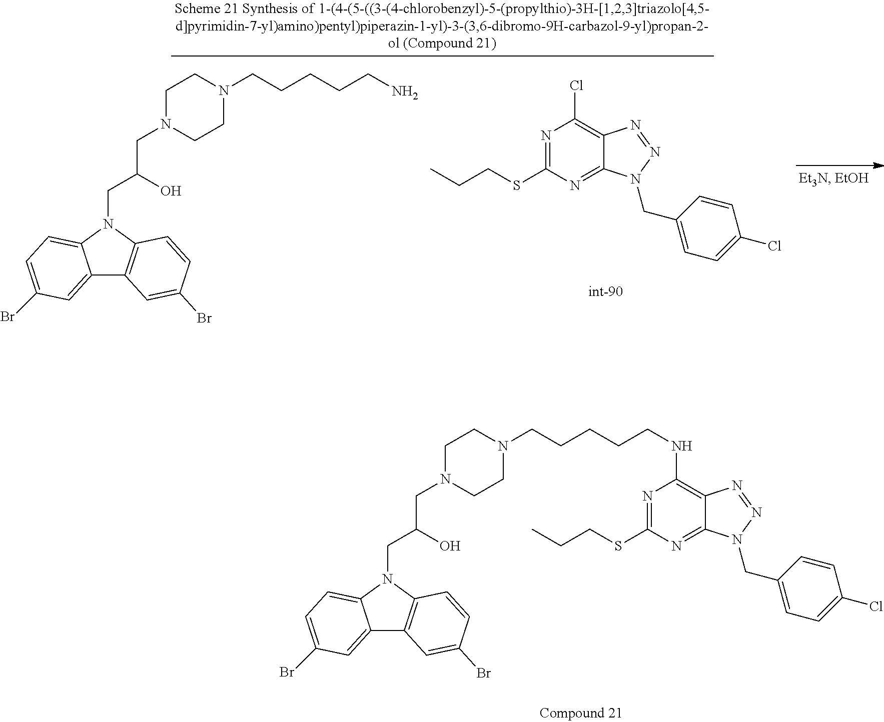 [SMILES: NCCCCCN1CCN(CC(O)Cn2c3ccc(Br)cc3c3cc(Br)ccc32)CC1 ; CCCSc1nc(Cl)c2nnn(Cc3ccc(Cl)cc3)c2n1 ; CCCSc1nc(NCCCCCN2CCN(CC(O)Cn3c4ccc(Br)cc4c4cc(Br)ccc43)CC2)c2nnn(Cc3ccc(Cl)cc3)c2n1]

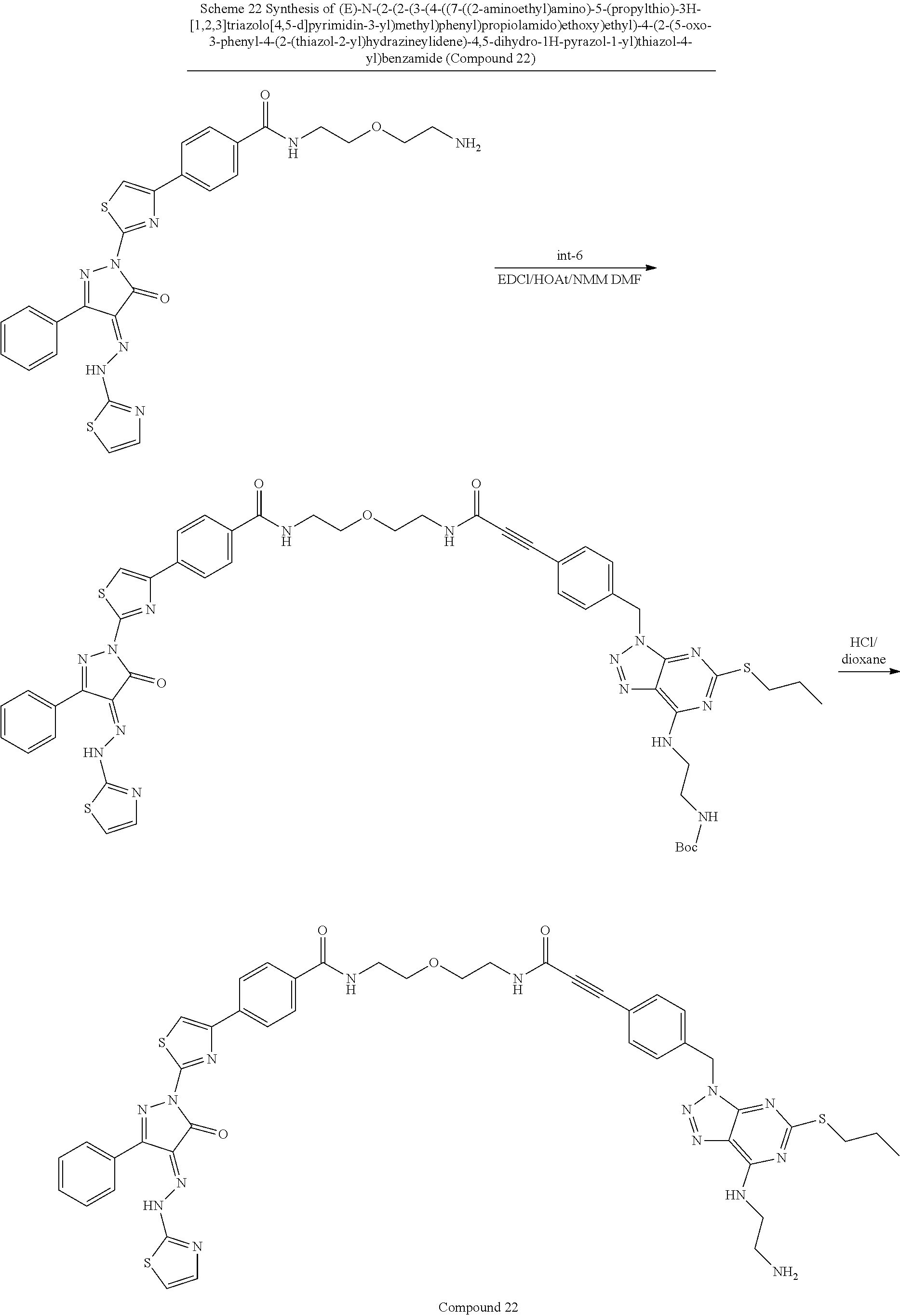 [SMILES: NCCOCCNC(=O)c1ccc(-c2csc(N3N=C(c4ccccc4)/C(=N\Nc4nccs4)C3=O)n2)cc1 ; CCCSc1nc(NCCNC(=O)OC(C)(C)C)c2nnn(Cc3ccc(C#CC(=O)NCCOCCNC(=O)c4ccc(-c5csc(N6N=C(c7ccccc7)/C(=N\Nc7nccs7)C6=O)n5)cc4)cc3)c2n1 ; CCCSc1nc(NCCN)c2nnn(Cc3ccc(C#CC(=O)NCCOCCNC(=O)c4ccc(-c5csc(N6N=C(c7ccccc7)/C(=N\Nc7nccs7)C6=O)n5)cc4)cc3)c2n1]

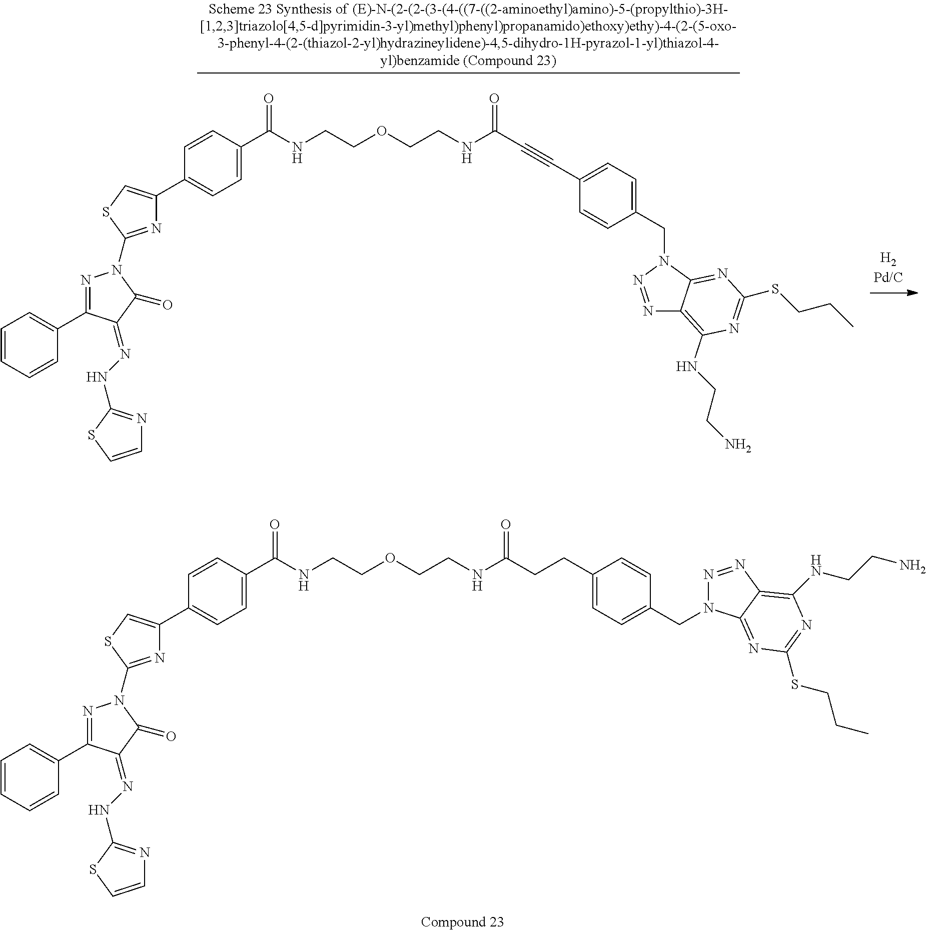 [SMILES: CCCSc1nc(NCCN)c2nnn(Cc3ccc(C#CC(=O)NCCOCCNC(=O)c4ccc(-c5csc(N6N=C(c7ccccc7)/C(=N\Nc7nccs7)C6=O)n5)cc4)cc3)c2n1 ; CCCSc1nc(NCCN)c2nnn(Cc3ccc(CCC(=O)NCCOCCNC(=O)c4ccc(-c5csc(N6N=C(c7ccccc7)/C(=N\Nc7nccs7)C6=O)n5)cc4)cc3)c2n1]

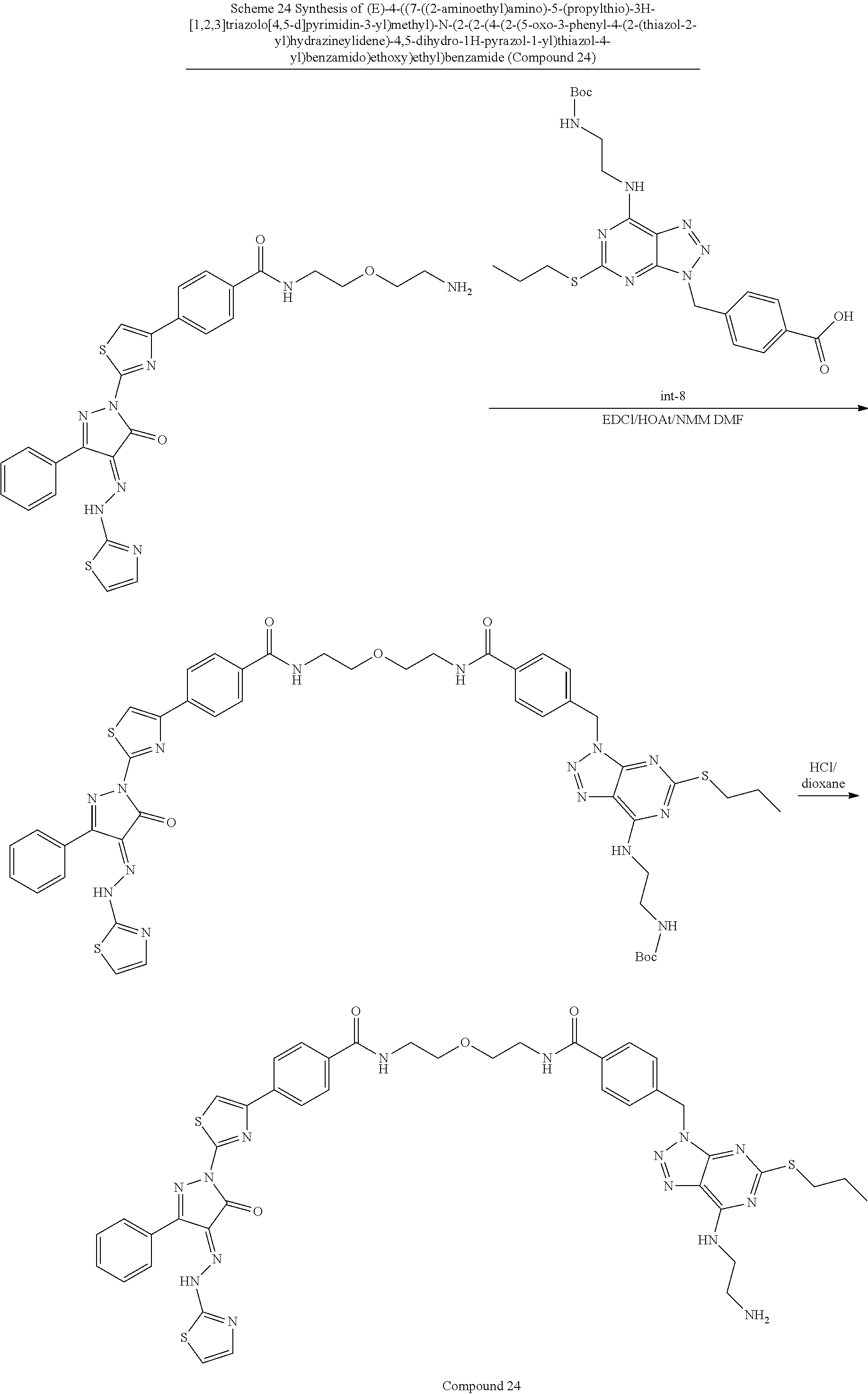 [SMILES: NCCOCCNC(=O)c1ccc(-c2csc(N3N=C(c4ccccc4)/C(=N\Nc4nccs4)C3=O)n2)cc1 ; CCCSc1nc(NCCNC(=O)OC(C)(C)C)c2nnn(Cc3ccc(C(=O)O)cc3)c2n1 ; CCCSc1nc(NCCNC(=O)OC(C)(C)C)c2nnn(Cc3ccc(C(=O)NCCOCCNC(=O)c4ccc(-c5csc(N6N=C(c7ccccc7)/C(=N\Nc7nccs7)C6=O)n5)cc4)cc3)c2n1 ; CCCSc1nc(NCCN)c2nnn(Cc3ccc(C(=O)NCCOCCNC(=O)c4ccc(-c5csc(N6N=C(c7ccccc7)/C(=N\Nc7nccs7)C6=O)n5)cc4)cc3)c2n1]

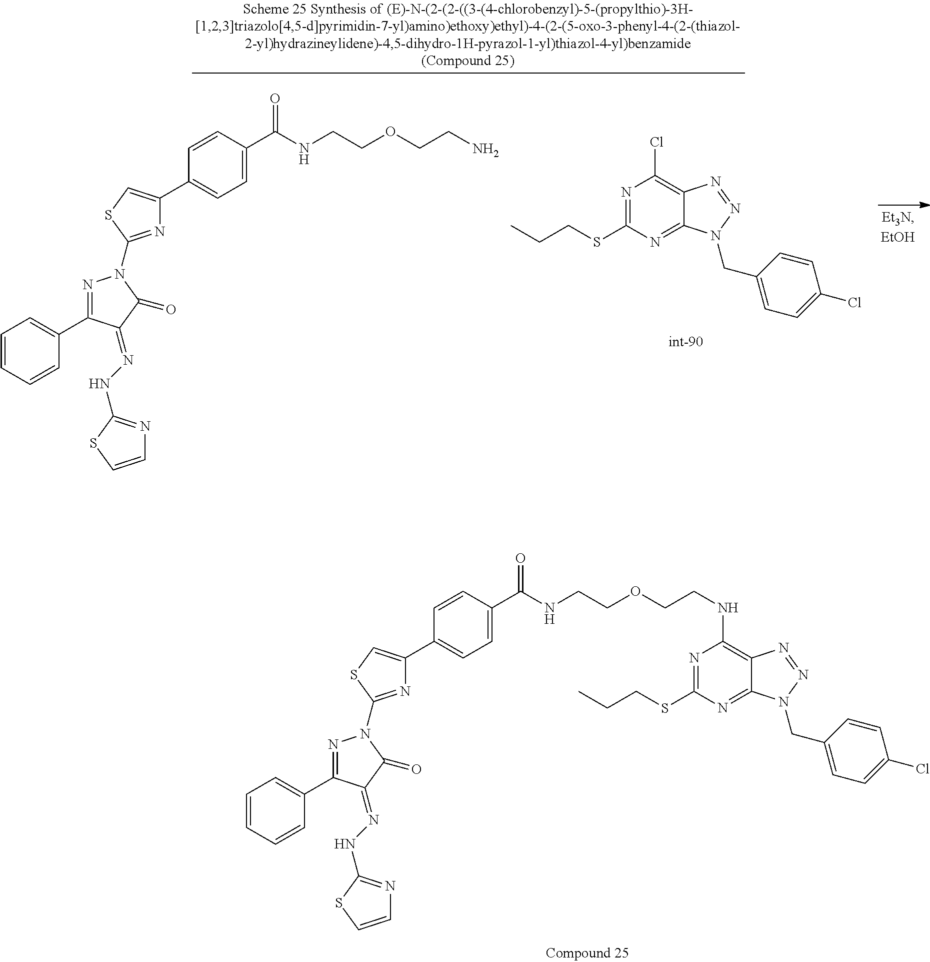 [SMILES: NCCOCCNC(=O)c1ccc(-c2csc(N3N=C(c4ccccc4)/C(=N\Nc4nccs4)C3=O)n2)cc1 ; CCCSc1nc(Cl)c2nnn(Cc3ccc(Cl)cc3)c2n1 ; CCCSc1nc(NCCOCCNC(=O)c2ccc(-c3csc(N4N=C(c5ccccc5)/C(=N\Nc5nccs5)C4=O)n3)cc2)c2nnn(Cc3ccc(Cl)cc3)c2n1]

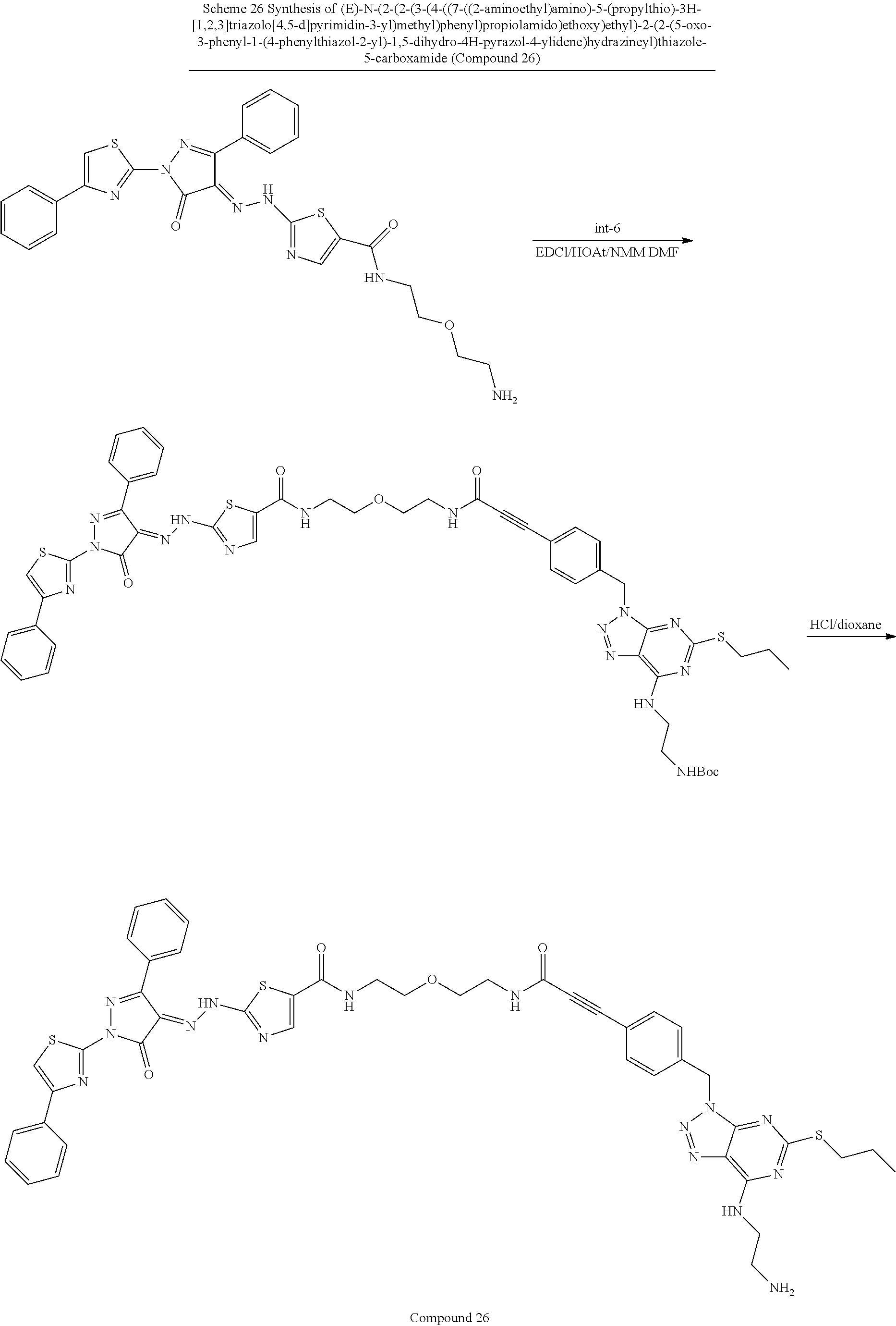 [SMILES: NCCOCCNC(=O)c1cnc(N/N=C2/C(=O)N(c3nc(-c4ccccc4)cs3)N=C2c2ccccc2)s1 ; CCCSc1nc(NCCNC(=O)OC(C)(C)C)c2nnn(Cc3ccc(C#CC(=O)NCCOCCNC(=O)c4cnc(N/N=C5/C(=O)N(c6nc(-c7ccccc7)cs6)N=C5c5ccccc5)s4)cc3)c2n1 ; CCCSc1nc(NCCN)c2nnn(Cc3ccc(C#CC(=O)NCCOCCNC(=O)c4cnc(N/N=C5/C(=O)N(c6nc(-c7ccccc7)cs6)N=C5c5ccccc5)s4)cc3)c2n1]

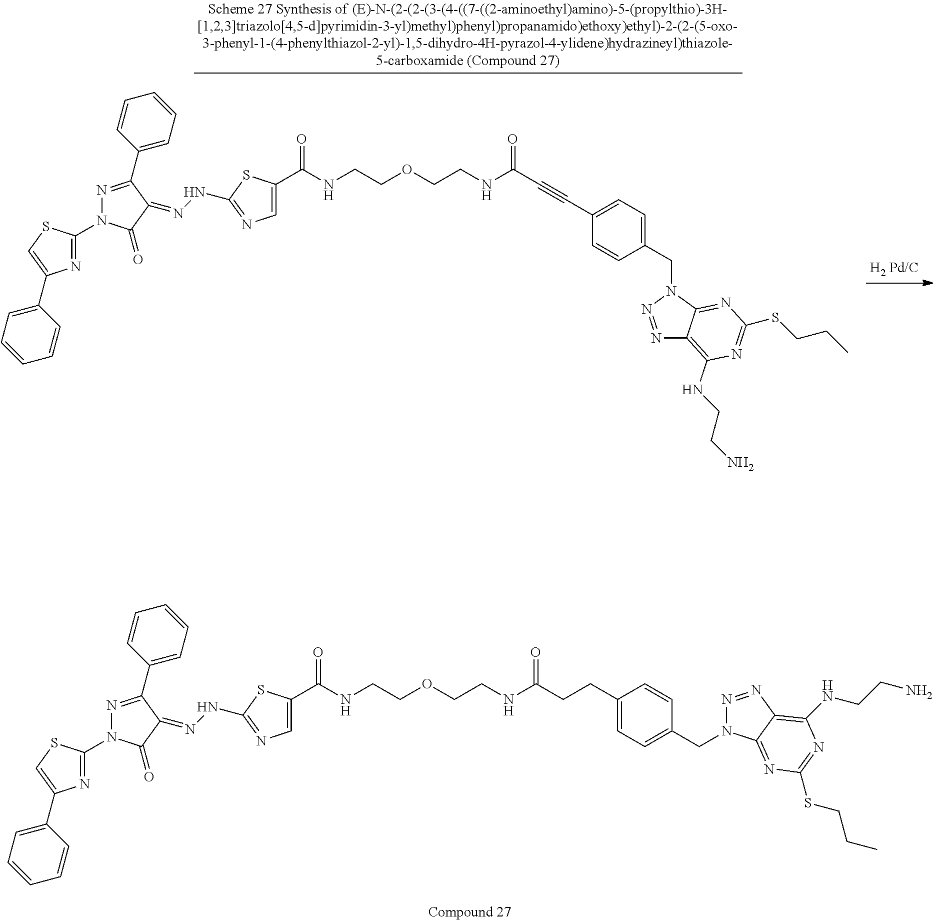 [SMILES: CCCSc1nc(NCCN)c2nnn(Cc3ccc(C#CC(=O)NCCOCCNC(=O)c4cnc(N/N=C5/C(=O)N(c6nc(-c7ccccc7)cs6)N=C5c5ccccc5)s4)cc3)c2n1 ; CCCSc1nc(NCCN)c2nnn(Cc3ccc(CCC(=O)NCCOCCNC(=O)c4cnc(N/N=C5/C(=O)N(c6nc(-c7ccccc7)cs6)N=C5c5ccccc5)s4)cc3)c2n1]

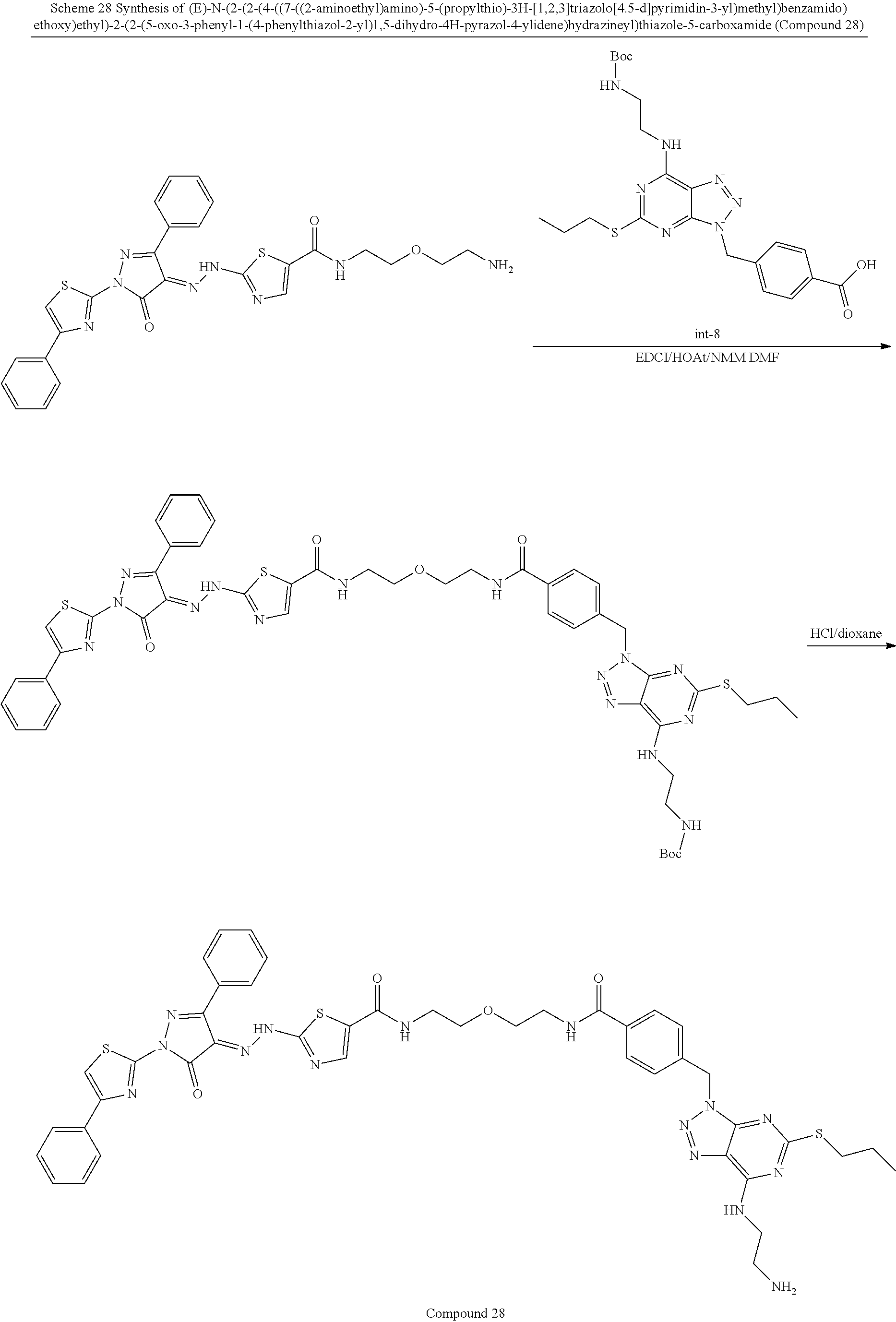 [SMILES: NCCOCCNC(=O)c1cnc(N/N=C2/C(=O)N(c3nc(-c4ccccc4)cs3)N=C2c2ccccc2)s1 ; CCCSc1nc(NCCNC(=O)OC(C)(C)C)c2nnn(Cc3ccc(C(=O)O)cc3)c2n1 ; CCCSc1nc(NCCNC(=O)OC(C)(C)C)c2nnn(Cc3ccc(C(=O)NCCOCCNC(=O)c4cnc(N/N=C5/C(=O)N(c6nc(-c7ccccc7)cs6)N=C5c5ccccc5)s4)cc3)c2n1 ; CCCSc1nc(NCCN)c2nnn(Cc3ccc(C(=O)NCCOCCNC(=O)c4cnc(N/N=C5/C(=O)N(c6nc(-c7ccccc7)cs6)N=C5c5ccccc5)s4)cc3)c2n1]

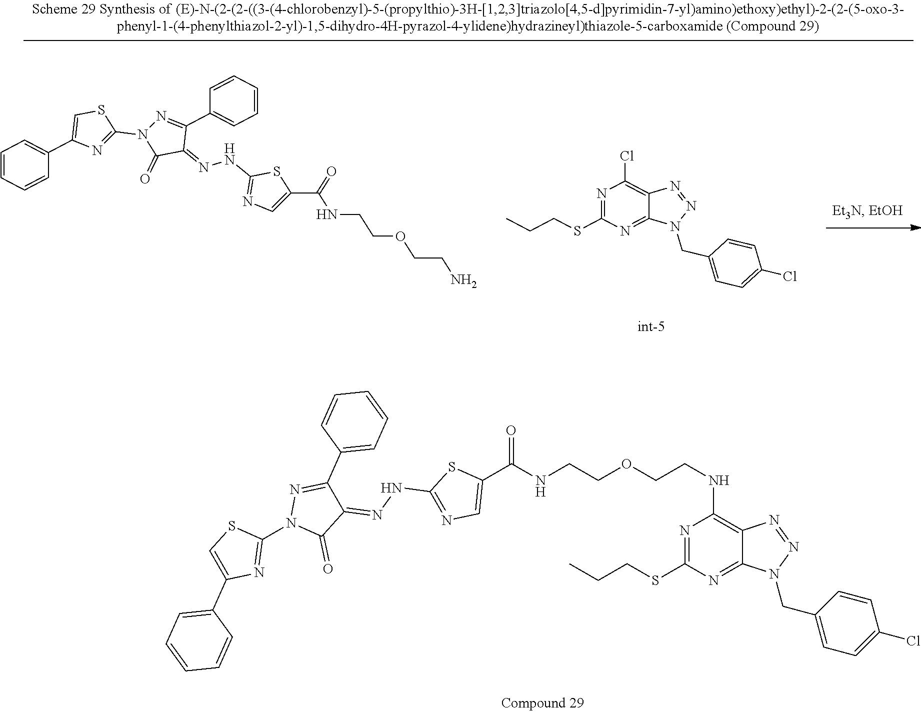 [SMILES: NCCOCCNC(=O)c1cnc(N/N=C2/C(=O)N(c3nc(-c4ccccc4)cs3)N=C2c2ccccc2)s1 ; CCCSc1nc(Cl)c2nnn(Cc3ccc(Cl)cc3)c2n1 ; CCCSc1nc(NCCOCCNC(=O)c2cnc(N/N=C3/C(=O)N(c4nc(-c5ccccc5)cs4)N=C3c3ccccc3)s2)c2nnn(Cc3ccc(Cl)cc3)c2n1]

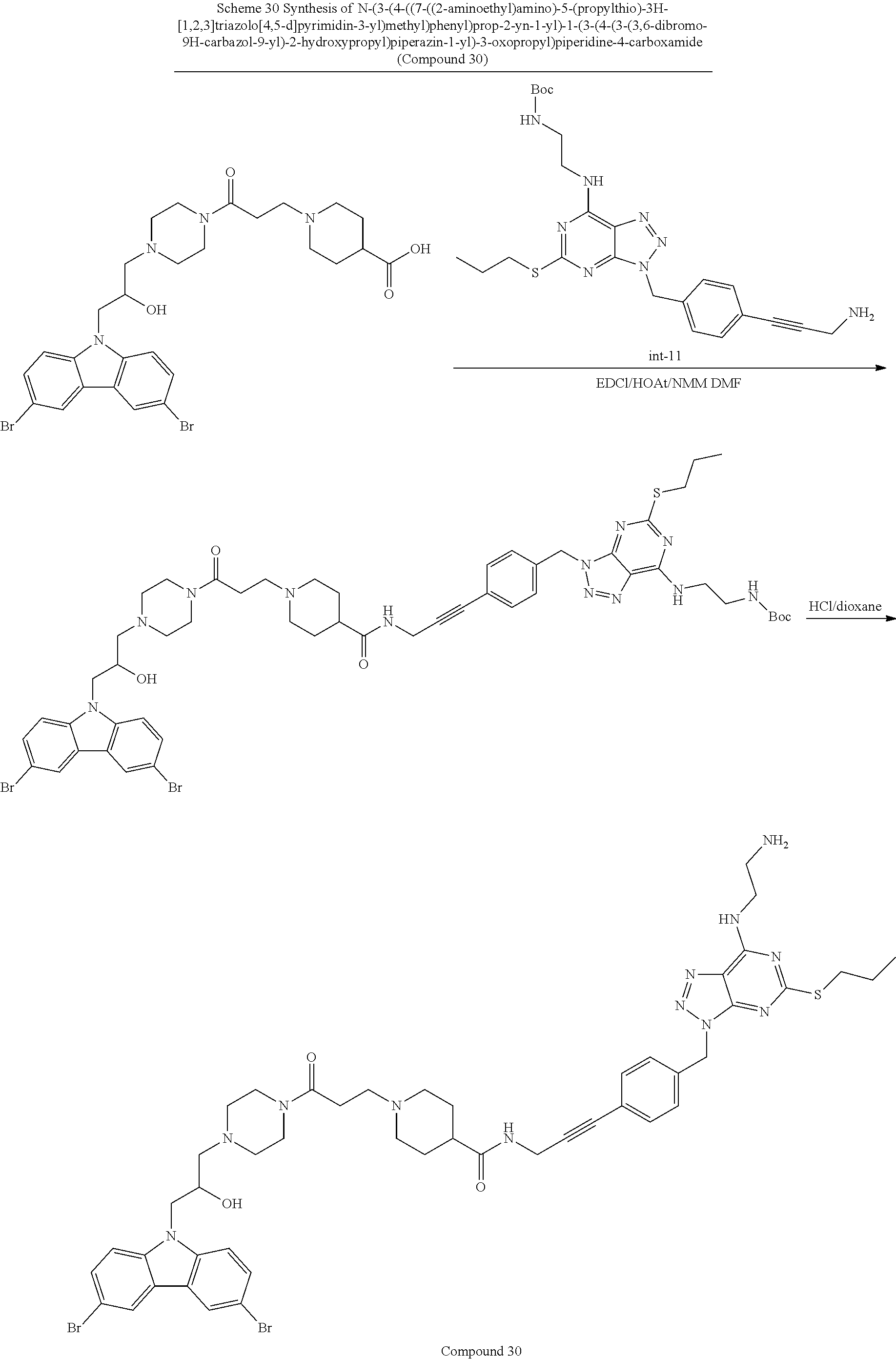

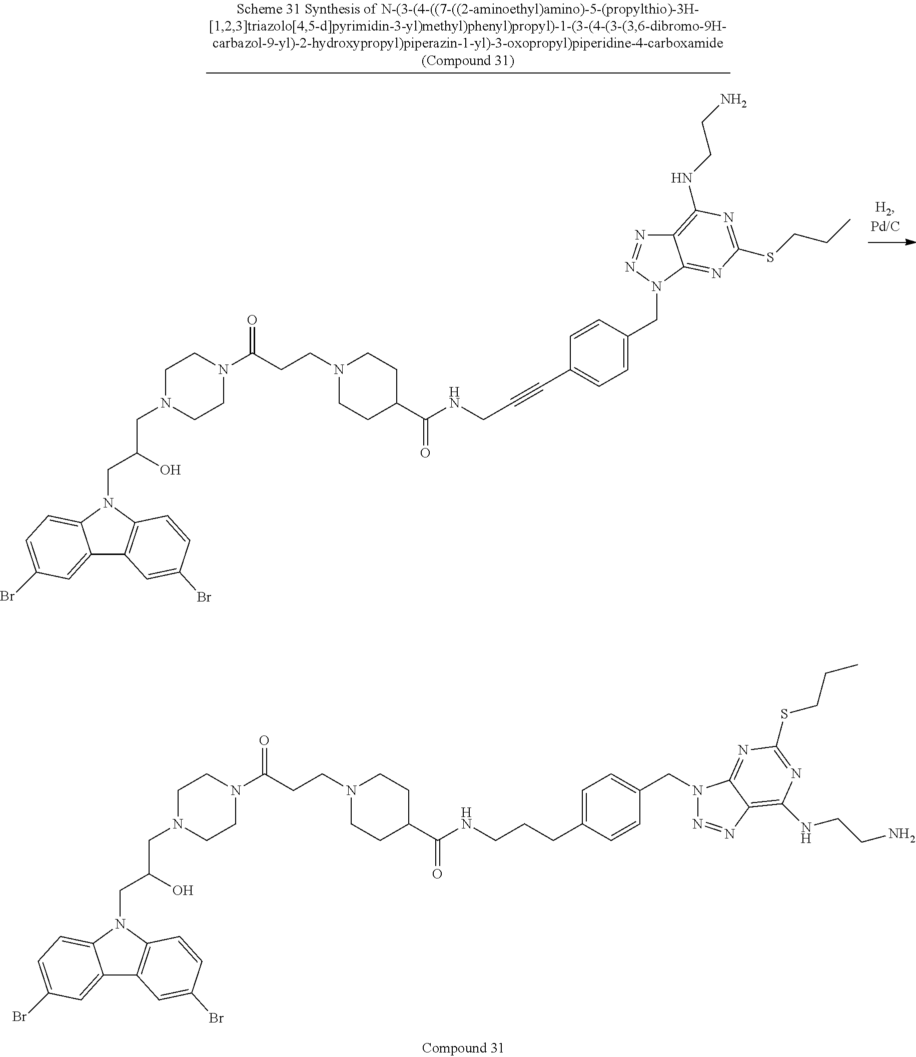 [SMILES: CCCSc1nc(NCCN)c2nnn(Cc3ccc(C#CCNC(=O)C4CCN(CCC(=O)N5CCN(CC(O)Cn6c7ccc(Br)cc7c7cc(Br)ccc76)CC5)CC4)cc3)c2n1 ; CCCSc1nc(NCCN)c2nnn(Cc3ccc(CCCNC(=O)C4CCN(CCC(=O)N5CCN(CC(O)Cn6c7ccc(Br)cc7c7cc(Br)ccc76)CC5)CC4)cc3)c2n1]

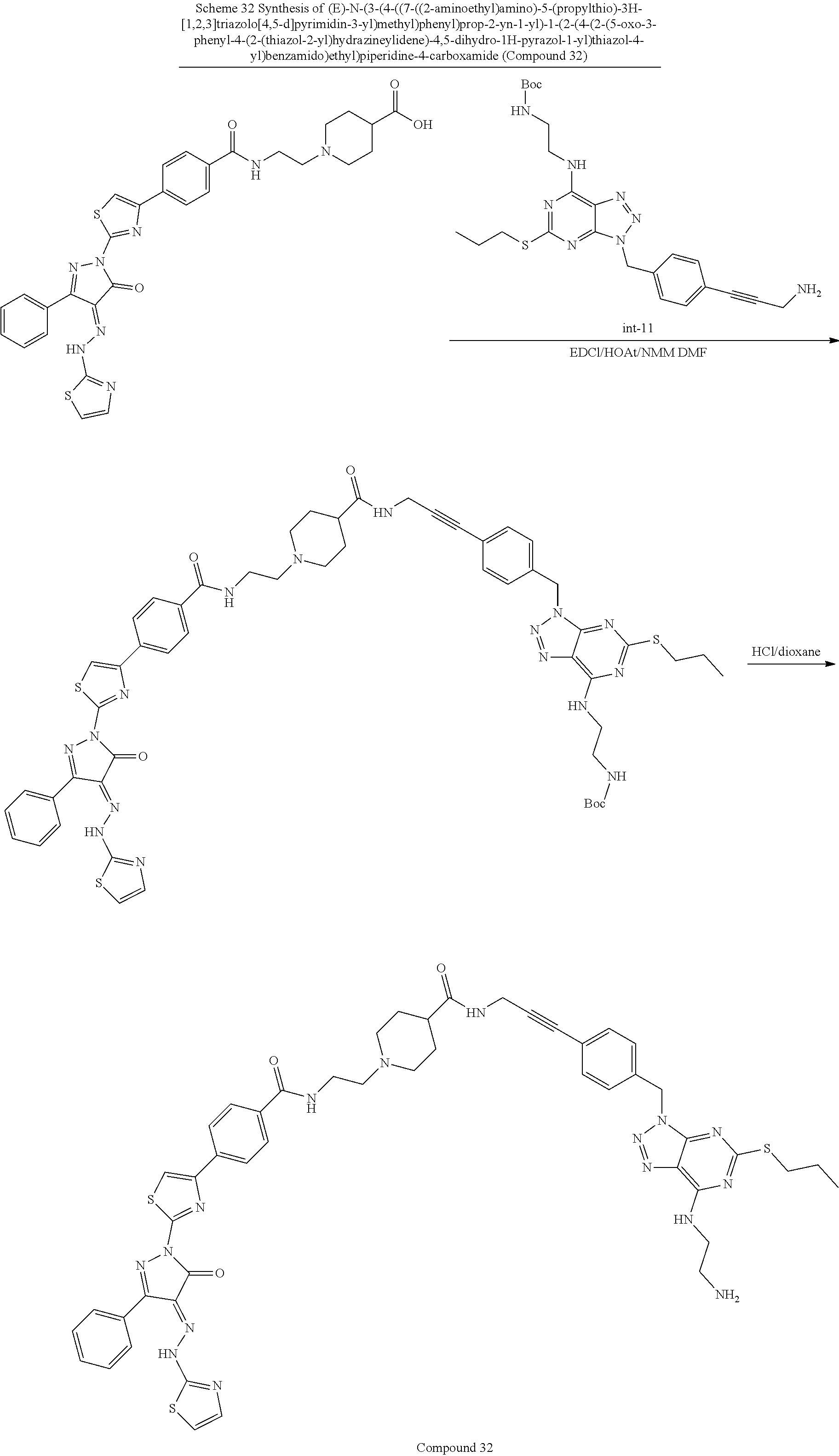 [SMILES: O=C(NCCN1CCC(C(=O)O)CC1)c1ccc(-c2csc(N3N=C(c4ccccc4)/C(=N\Nc4nccs4)C3=O)n2)cc1 ; CCCSc1nc(NCCNC(=O)OC(C)(C)C)c2nnn(Cc3ccc(C#CCN)cc3)c2n1 ; CCCSc1nc(NCCNC(=O)OC(C)(C)C)c2nnn(Cc3ccc(C#CCNC(=O)C4CCN(CCNC(=O)c5ccc(-c6csc(N7N=C(c8ccccc8)/C(=N\Nc8nccs8)C7=O)n6)cc5)CC4)cc3)c2n1 ; CCCSc1nc(NCCN)c2nnn(Cc3ccc(C#CCNC(=O)C4CCN(CCNC(=O)c5ccc(-c6csc(N7N=C(c8ccccc8)/C(=N\Nc8nccs8)C7=O)n6)cc5)CC4)cc3)c2n1]

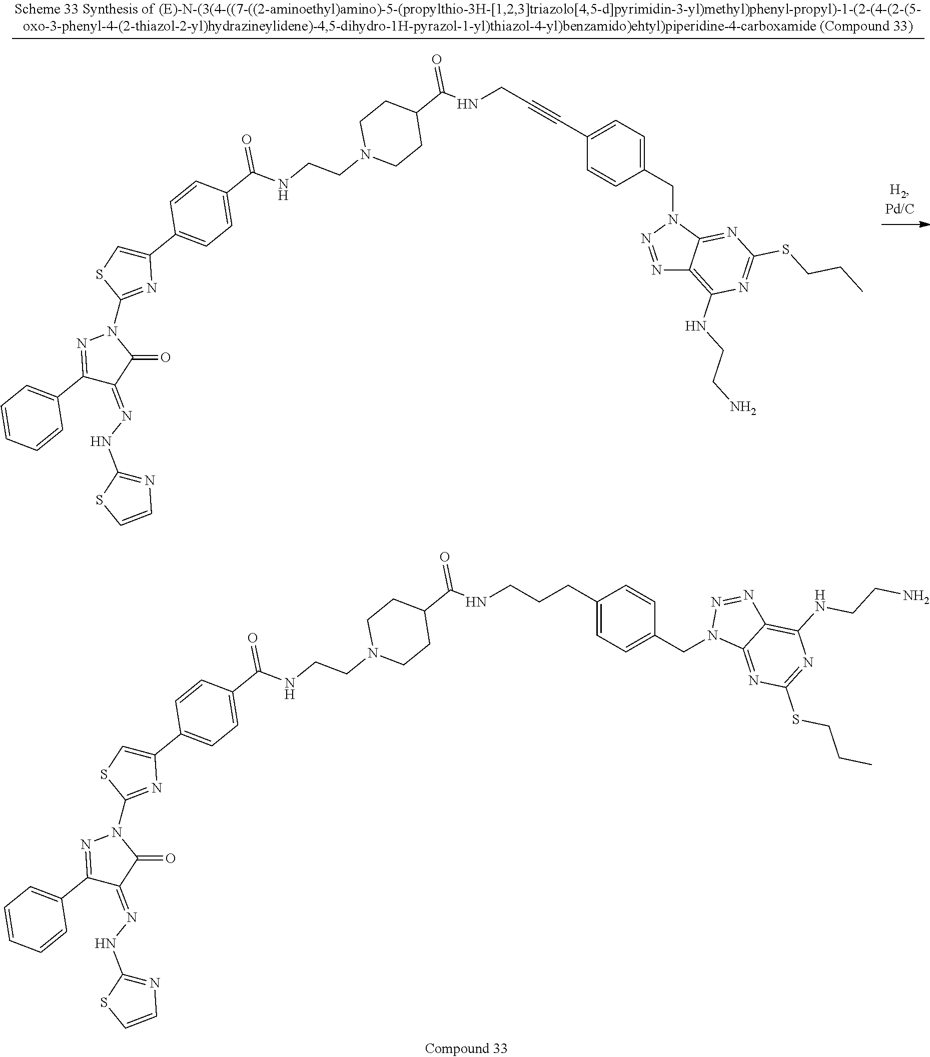 [SMILES: CCCSc1nc(NCCN)c2nnn(Cc3ccc(C#CCNC(=O)C4CCN(CCNC(=O)c5ccc(-c6csc(N7N=C(c8ccccc8)/C(=N\Nc8nccs8)C7=O)n6)cc5)CC4)cc3)c2n1 ; CCCSc1nc(NCCN)c2nnn(Cc3ccc(CCCNC(=O)C4CCN(CCNC(=O)c5ccc(-c6csc(N7N=C(c8ccccc8)/C(=N\Nc8nccs8)C7=O)n6)cc5)CC4)cc3)c2n1]

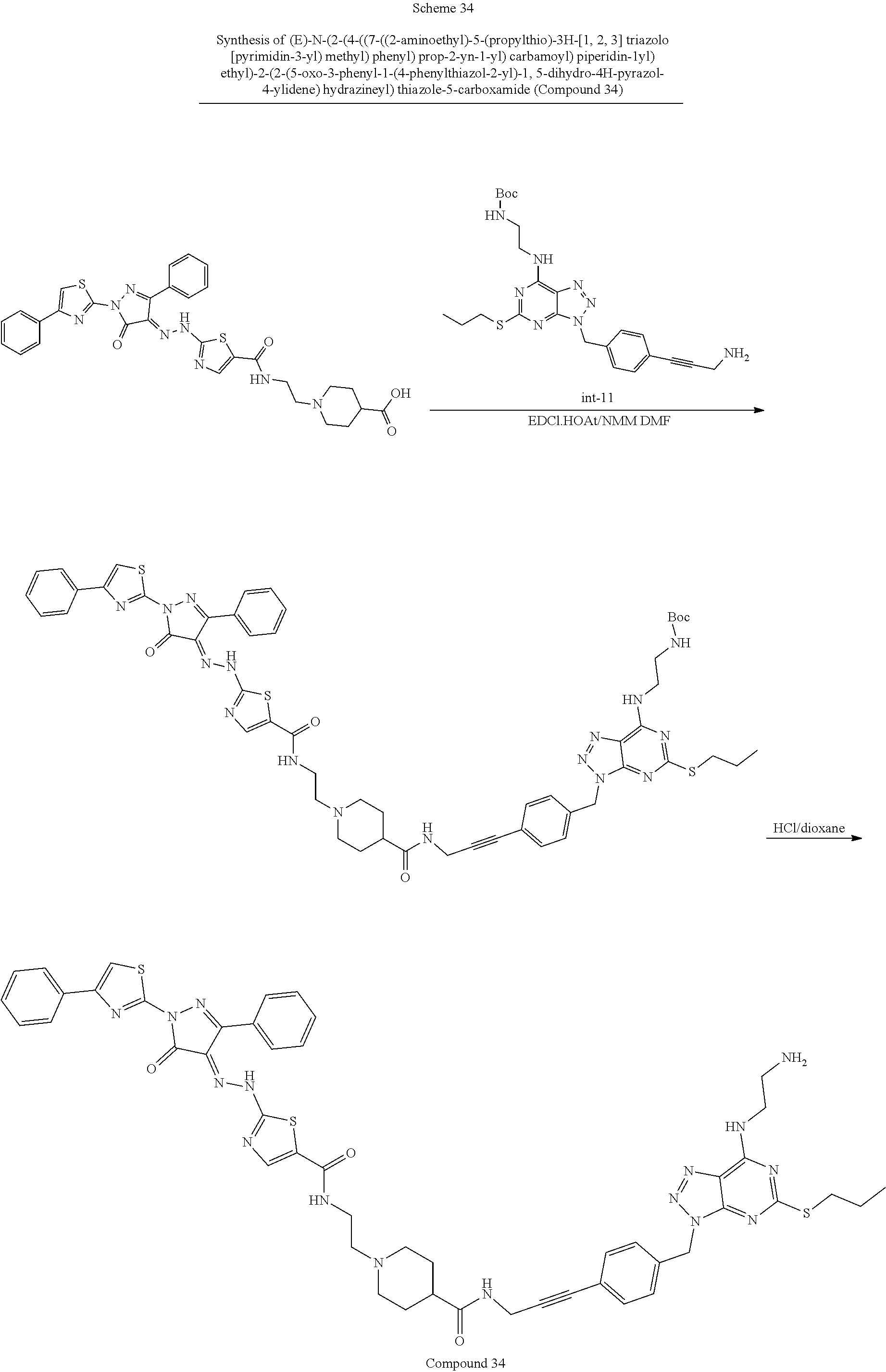 [SMILES: O=C(NCCN1CCC(C(=O)O)CC1)c1cnc(N/N=C2/C(=O)N(c3nc(-c4ccccc4)cs3)N=C2c2ccccc2)s1 ; CCCSc1nc(NCCNC(=O)OC(C)(C)C)c2nnn(Cc3ccc(C#CCN)cc3)c2n1 ; CCCSc1nc(NCCNC(=O)OC(C)(C)C)c2nnn(Cc3ccc(C#CCNC(=O)C4CCN(CCNC(=O)c5cnc(N/N=C6/C(=O)N(c7nc(-c8ccccc8)cs7)N=C6c6ccccc6)s5)CC4)cc3)c2n1 ; CCCSc1nc(NCCN)c2nnn(Cc3ccc(C#CCNC(=O)C4CCN(CCNC(=O)c5cnc(N/N=C6/C(=O)N(c7nc(-c8ccccc8)cs7)N=C6c6ccccc6)s5)CC4)cc3)c2n1]

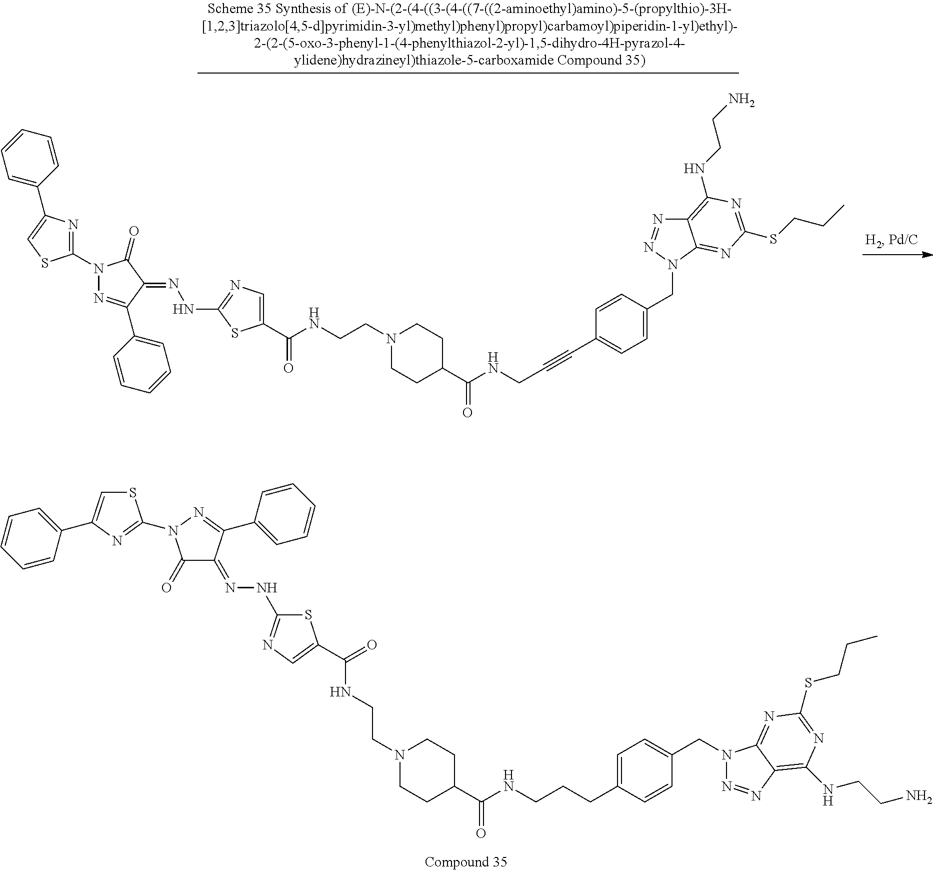 [SMILES: CCCSc1nc(NCCN)c2nnn(Cc3ccc(C#CCNC(=O)C4CCN(CCNC(=O)c5cnc(N/N=C6/C(=O)N(c7nc(-c8ccccc8)cs7)N=C6c6ccccc6)s5)CC4)cc3)c2n1 ; CCCSc1nc(NCCN)c2nnn(Cc3ccc(CCCNC(=O)C4CCN(CCNC(=O)c5cnc(N/N=C6/C(=O)N(c7nc(-c8ccccc8)cs7)N=C6c6ccccc6)s5)CC4)cc3)c2n1]

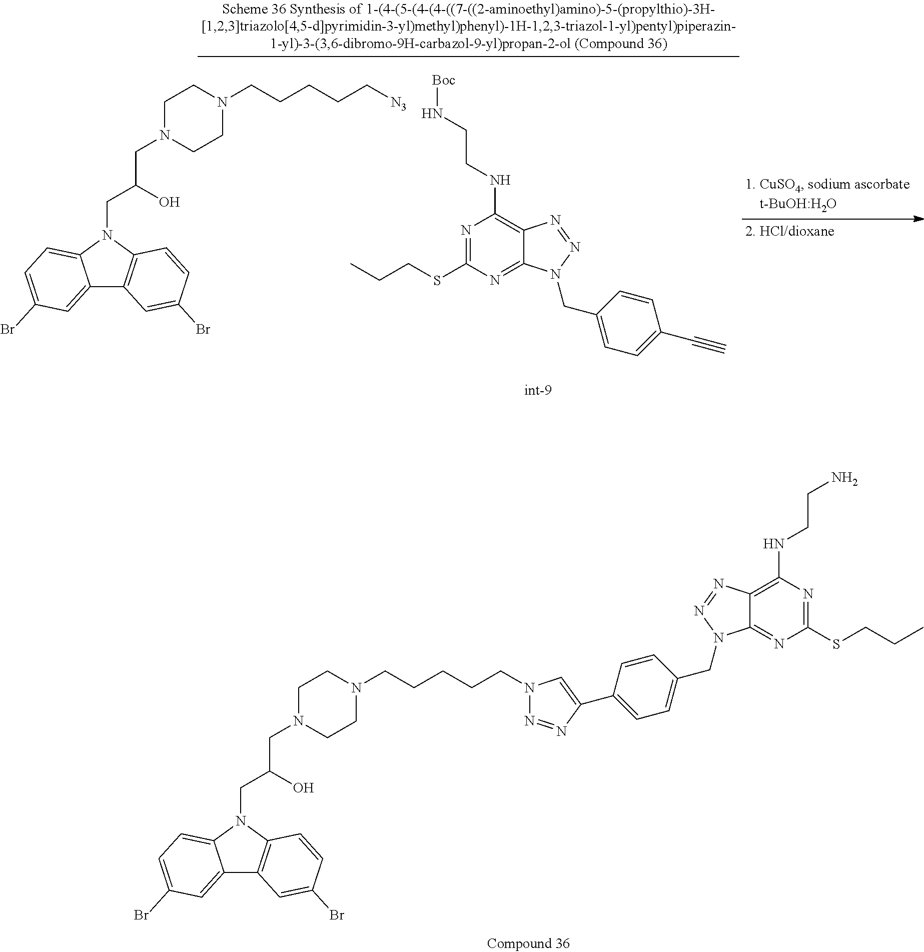

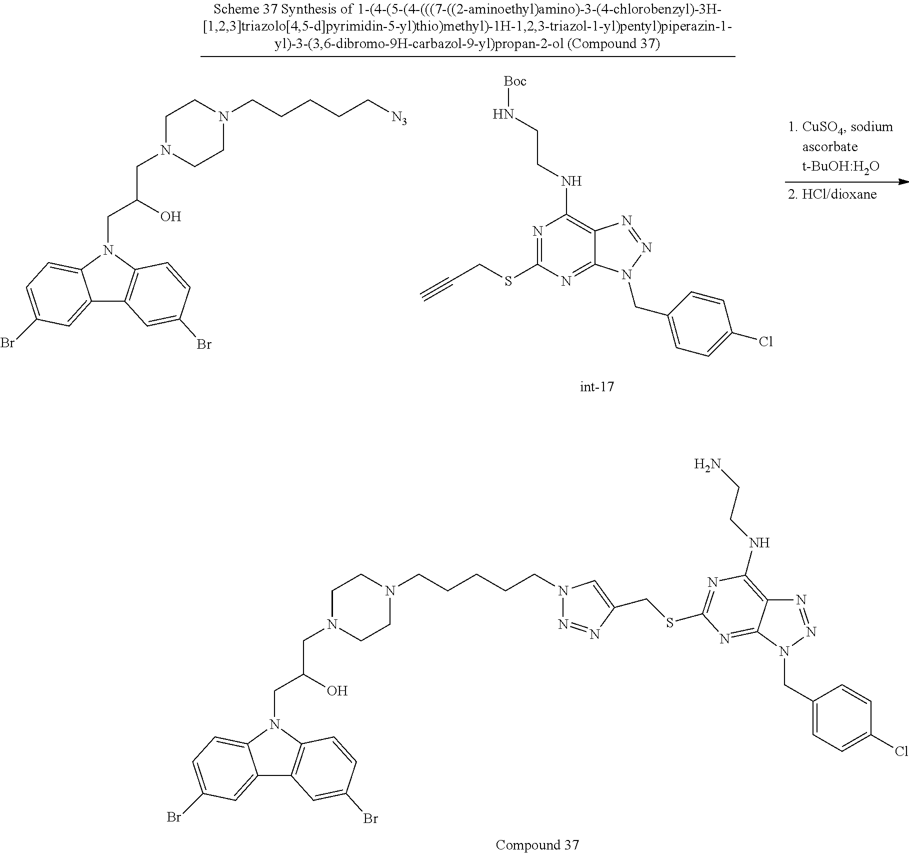 [SMILES: [N-]=[N+]=NCCCCCN1CCN(CC(O)Cn2c3ccc(Br)cc3c3cc(Br)ccc32)CC1 ; C#CCSc1nc(NCCNC(=O)OC(C)(C)C)c2nnn(Cc3ccc(Cl)cc3)c2n1 ; NCCNc1nc(SCc2cn(CCCCCN3CCN(CC(O)Cn4c5ccc(Br)cc5c5cc(Br)ccc54)CC3)nn2)nc2c1nnn2Cc1ccc(Cl)cc1]

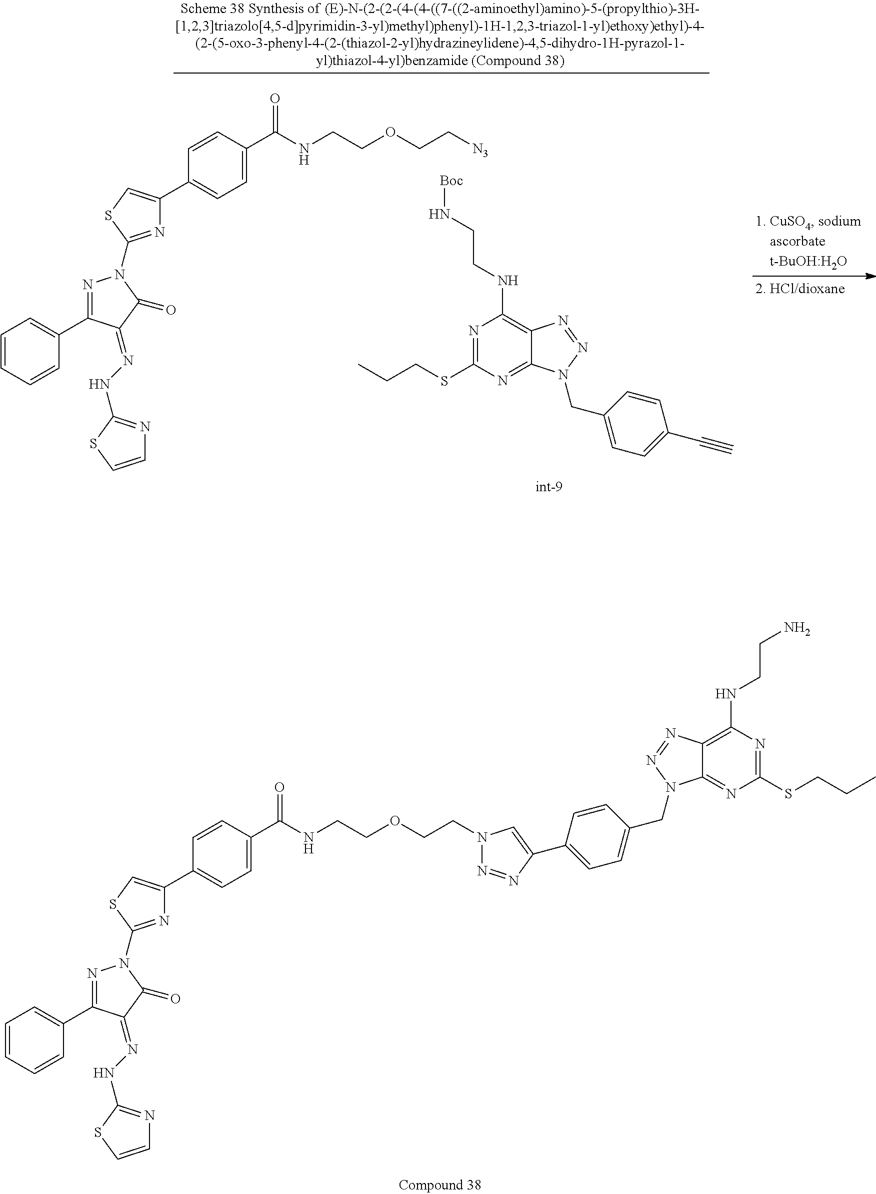

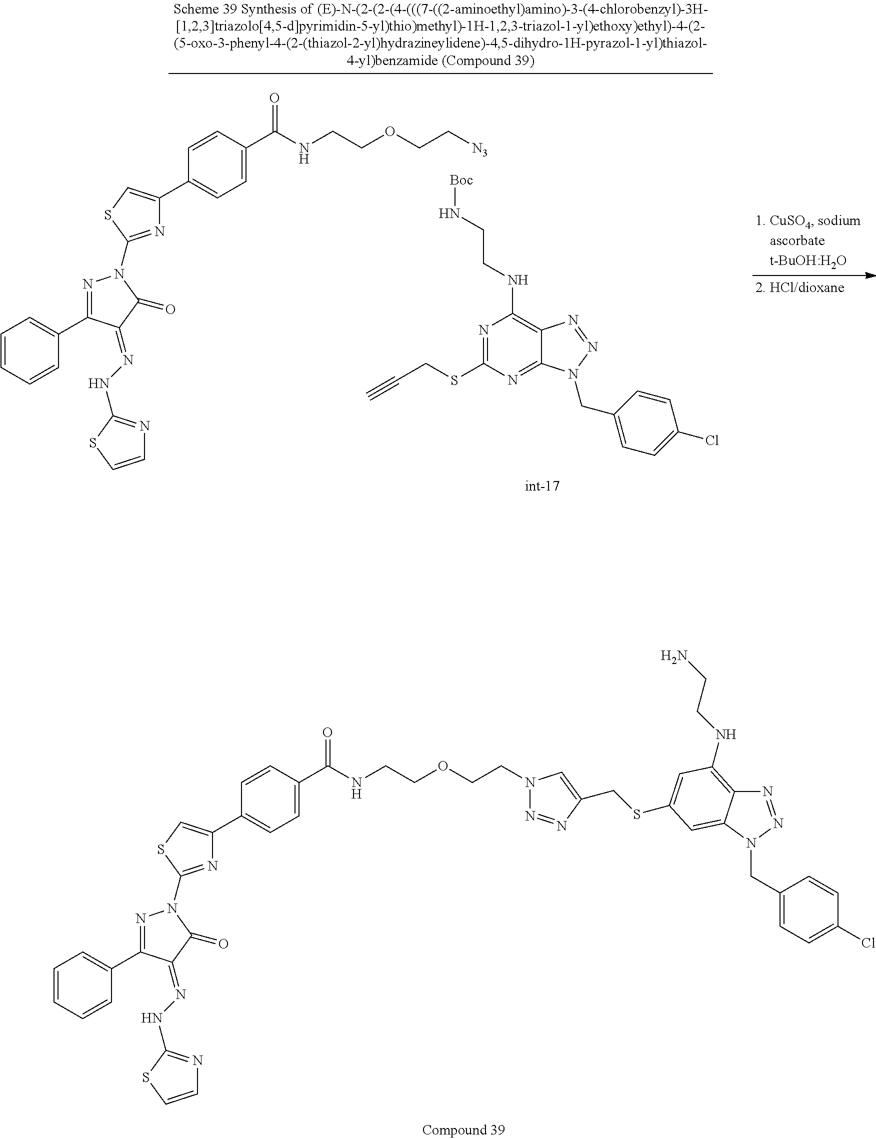

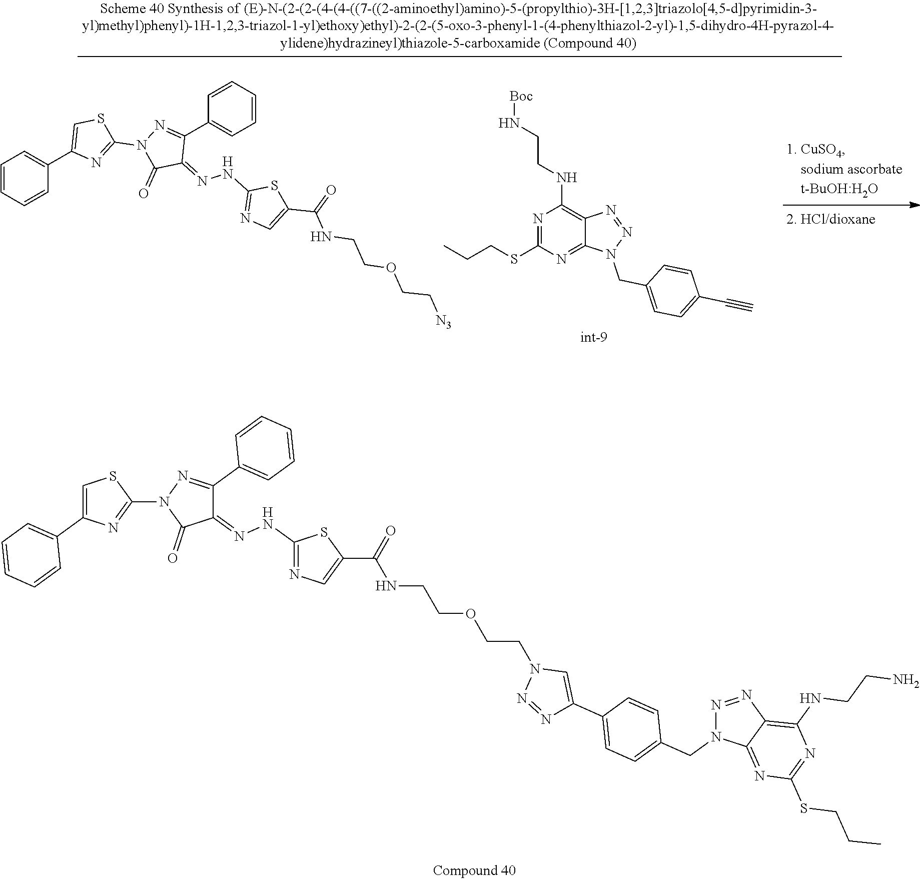 [SMILES: [N-]=[N+]=NCCOCCNC(=O)c1cnc(N/N=C2/C(=O)N(c3nc(-c4ccccc4)cs3)N=C2c2ccccc2)s1 ; C#Cc1ccc(Cn2nnc3c(NCCNC(=O)OC(C)(C)C)nc(SCCC)nc32)cc1 ; CCCSc1nc(NCCN)c2nnn(Cc3ccc(-c4cn(CCOCCNC(=O)c5cnc(N/N=C6/C(=O)N(c7nc(-c8ccccc8)cs7)N=C6c6ccccc6)s5)nn4)cc3)c2n1]

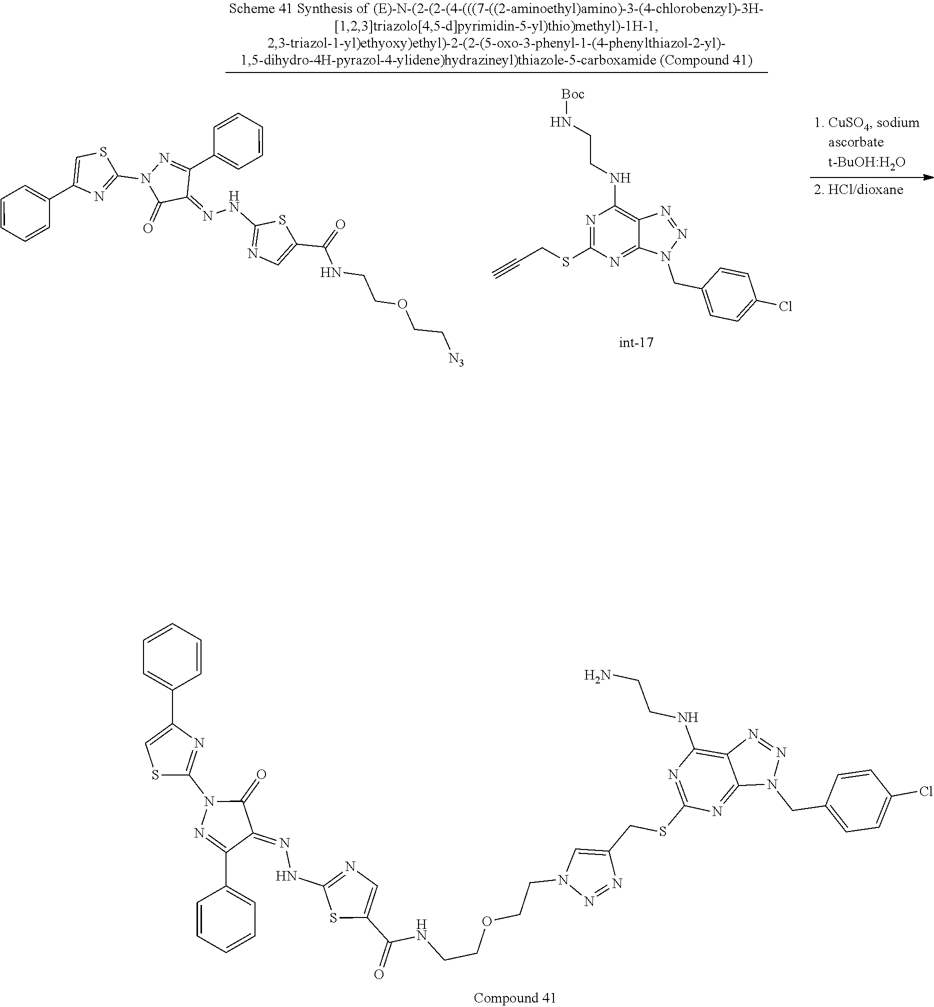 [SMILES: [N-]=[N+]=NCCOCCNC(=O)c1cnc(N/N=C2/C(=O)N(c3nc(-c4ccccc4)cs3)N=C2c2ccccc2)s1 ; C#CCSc1nc(NCCNC(=O)OC(C)(C)C)c2nnn(Cc3ccc(Cl)cc3)c2n1 ; NCCNc1nc(SCc2cn(CCOCCNC(=O)c3cnc(N/N=C4/C(=O)N(c5nc(-c6ccccc6)cs5)N=C4c4ccccc4)s3)nn2)nc2c1nnn2Cc1ccc(Cl)cc1]

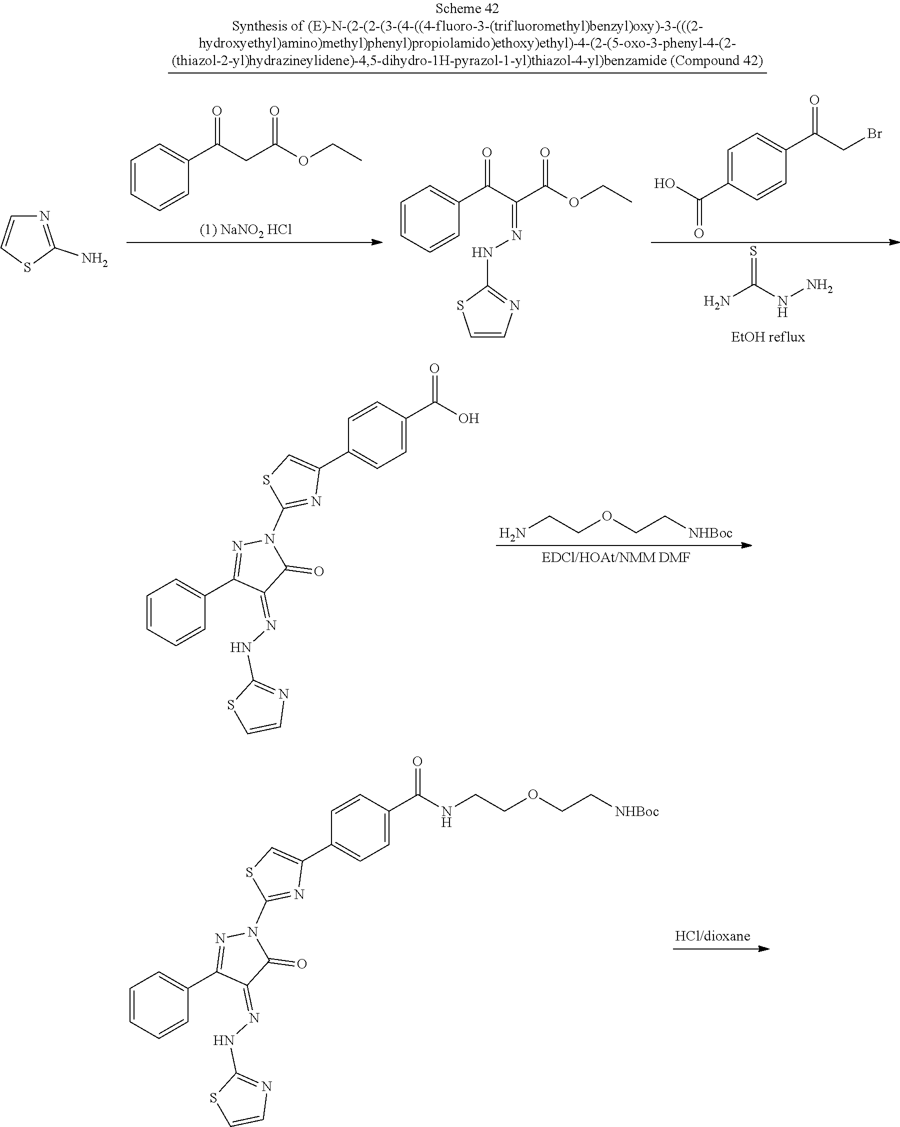

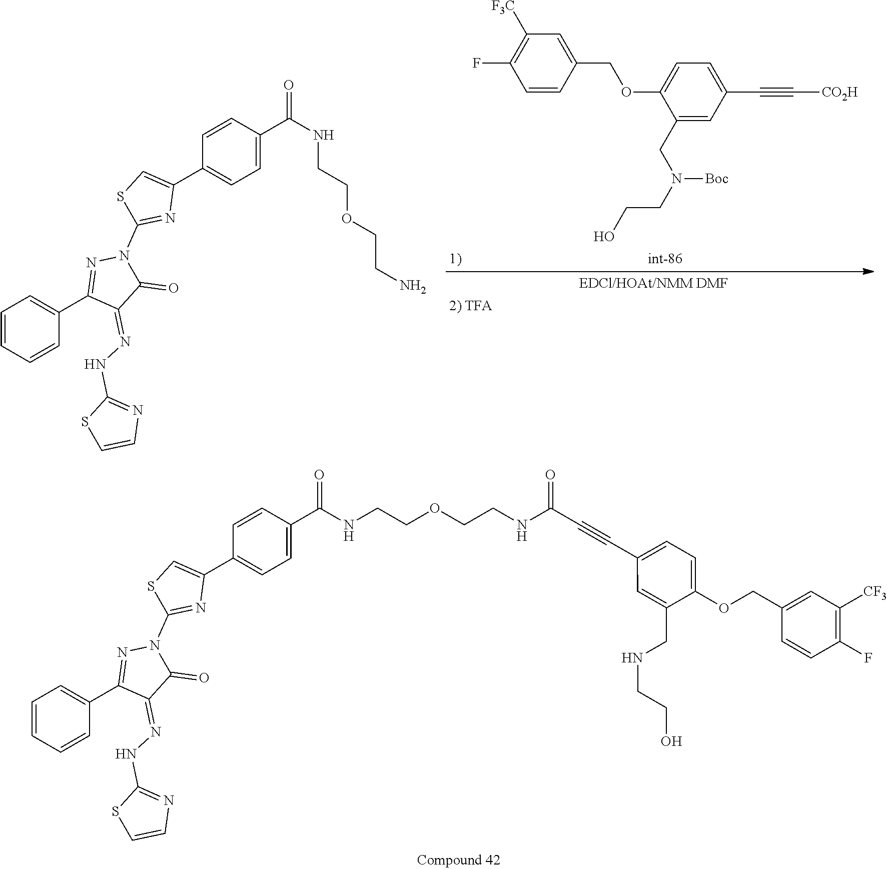 [SMILES: NCCOCCNC(=O)c1ccc(-c2csc(N3N=C(c4ccccc4)/C(=N\Nc4nccs4)C3=O)n2)cc1 ; CC(C)(C)OC(=O)N(CCO)Cc1cc(C#CC(=O)O)ccc1OCc1ccc(F)c(C(F)(F)F)c1 ; O=C(C#Cc1ccc(OCc2ccc(F)c(C(F)(F)F)c2)c(CNCCO)c1)NCCOCCNC(=O)c1ccc(-c2csc(N3N=C(c4ccccc4)/C(=N\Nc4nccs4)C3=O)n2)cc1]

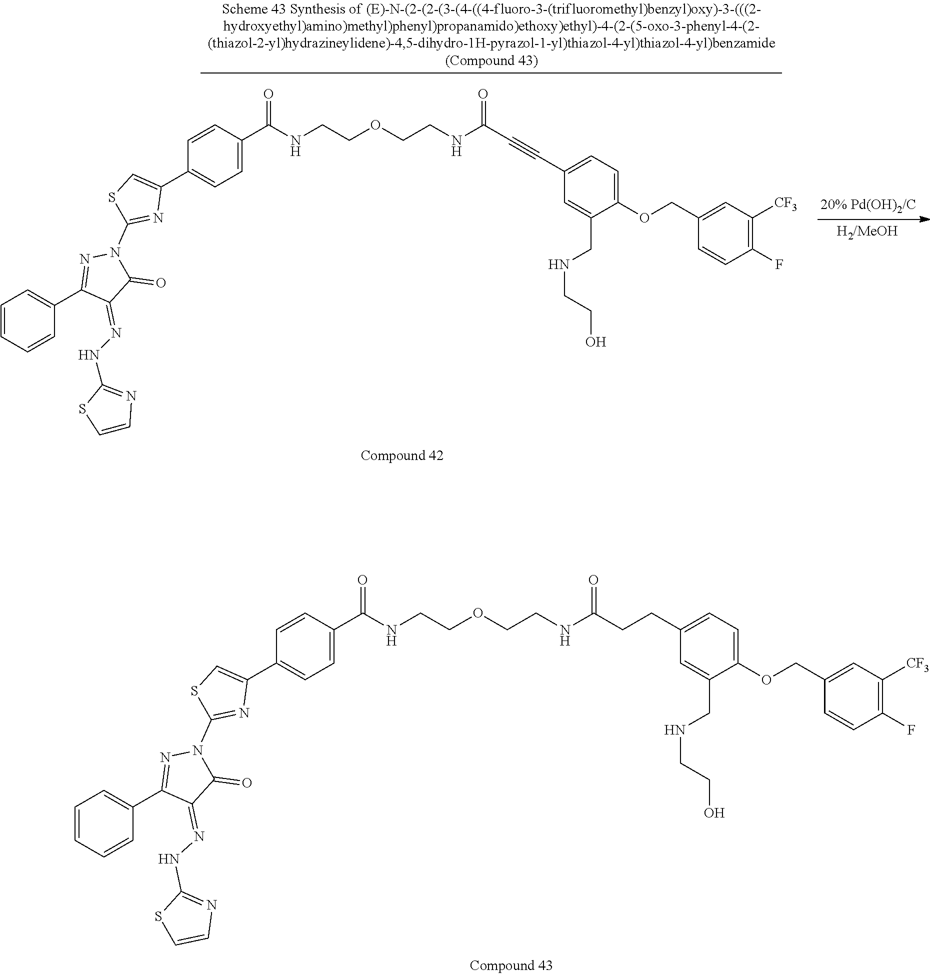 [SMILES: O=C(C#Cc1ccc(OCc2ccc(F)c(C(F)(F)F)c2)c(CNCCO)c1)NCCOCCNC(=O)c1ccc(-c2csc(N3N=C(c4ccccc4)/C(=N\Nc4nccs4)C3=O)n2)cc1 ; O=C(CCc1ccc(OCc2ccc(F)c(C(F)(F)F)c2)c(CNCCO)c1)NCCOCCNC(=O)c1ccc(-c2csc(N3N=C(c4ccccc4)/C(=N\Nc4nccs4)C3=O)n2)cc1]

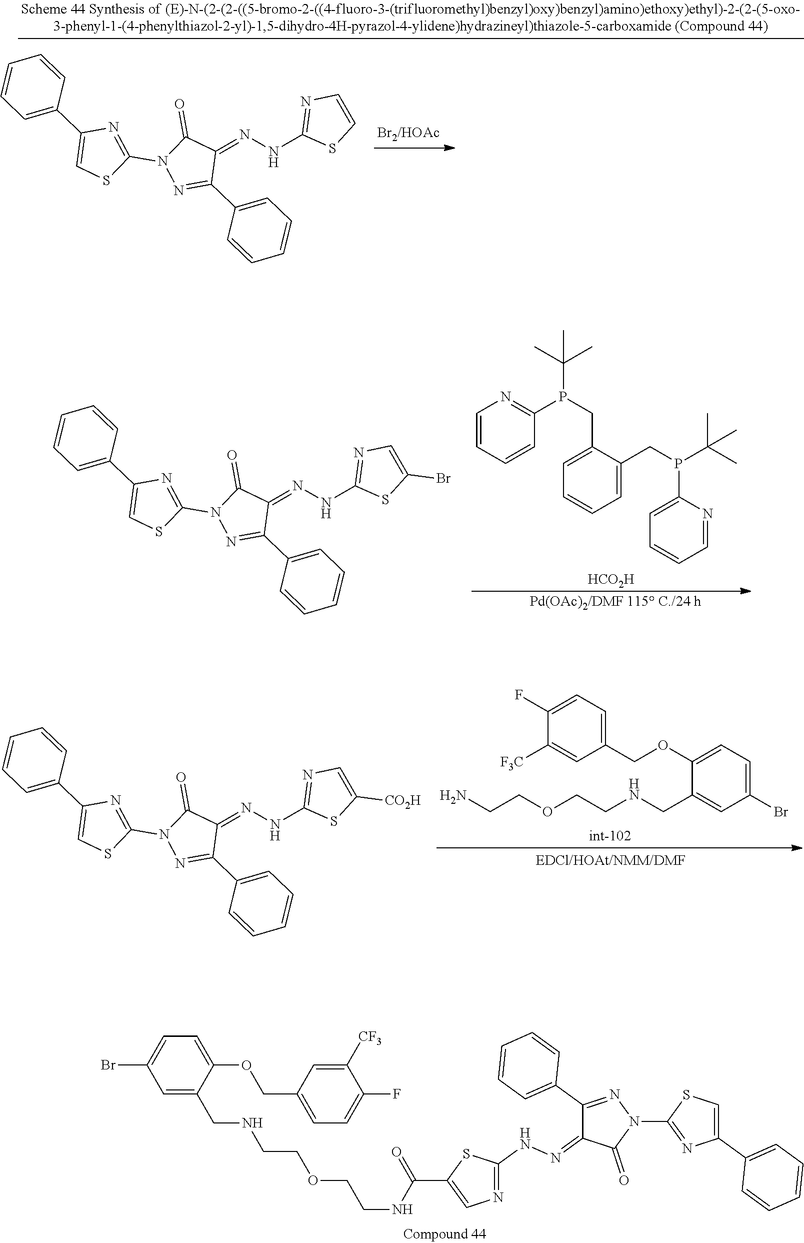

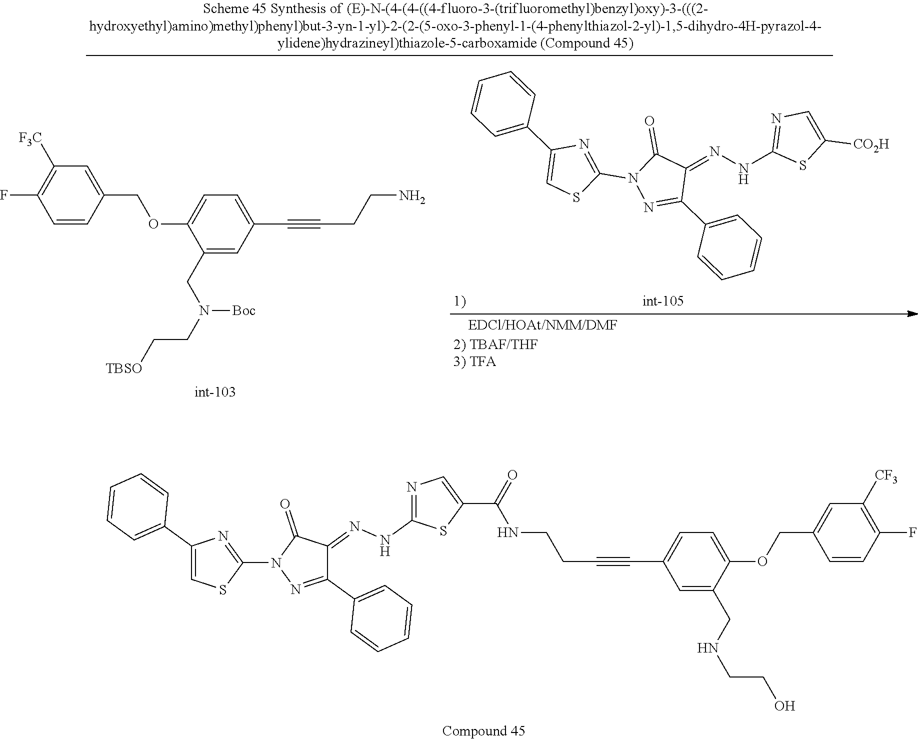

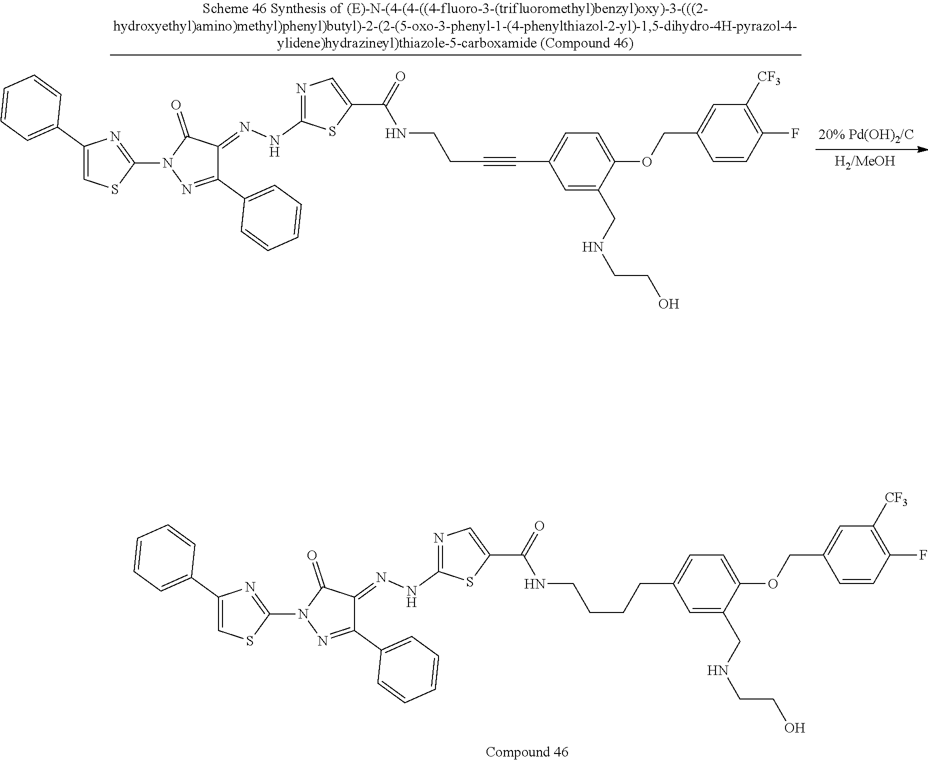 [SMILES: O=C(NCCC#Cc1ccc(OCc2ccc(F)c(C(F)(F)F)c2)c(CNCCO)c1)c1cnc(N/N=C2/C(=O)N(c3nc(-c4ccccc4)cs3)N=C2c2ccccc2)s1 ; O=C(NCCCCc1ccc(OCc2ccc(F)c(C(F)(F)F)c2)c(CNCCO)c1)c1cnc(N/N=C2/C(=O)N(c3nc(-c4ccccc4)cs3)N=C2c2ccccc2)s1]

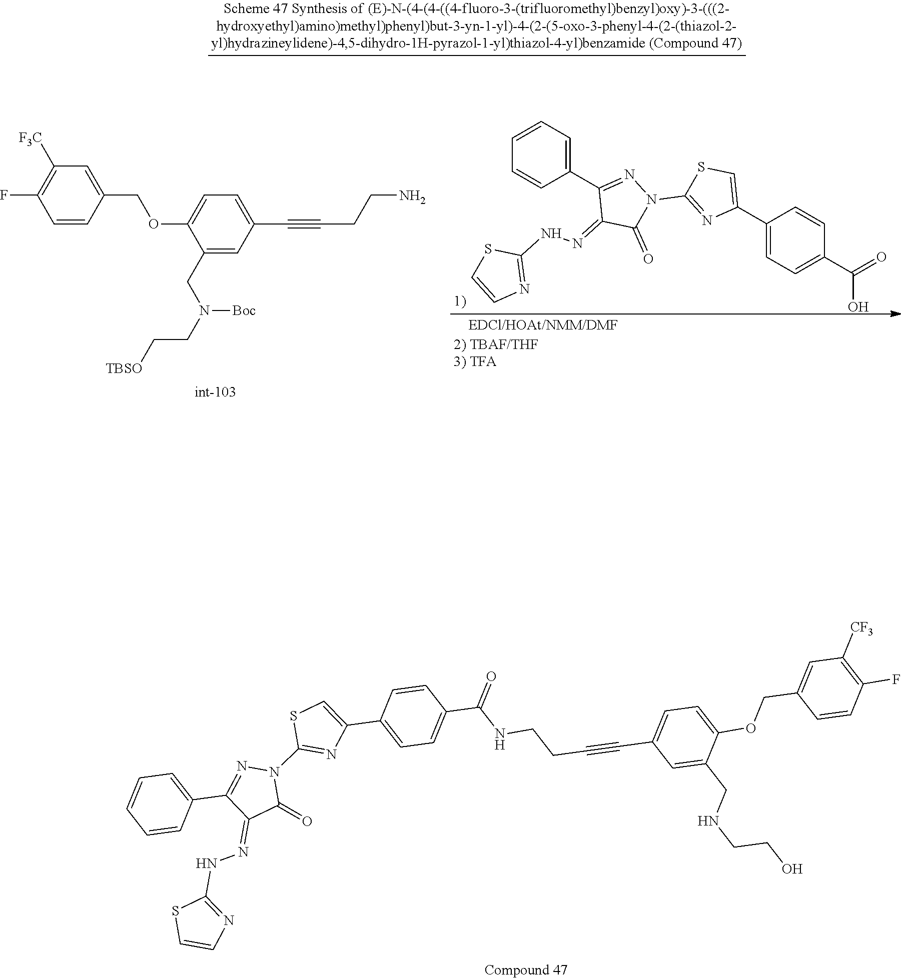 [SMILES: CC(C)(C)OC(=O)N(CCO[Si](C)(C)C(C)(C)C)Cc1cc(C#CCCN)ccc1OCc1ccc(F)c(C(F)(F)F)c1 ; O=C(O)c1ccc(-c2csc(N3N=C(c4ccccc4)/C(=N\Nc4nccs4)C3=O)n2)cc1 ; O=C(NCCC#Cc1ccc(OCc2ccc(F)c(C(F)(F)F)c2)c(CNCCO)c1)c1ccc(-c2csc(N3N=C(c4ccccc4)/C(=N\Nc4nccs4)C3=O)n2)cc1]

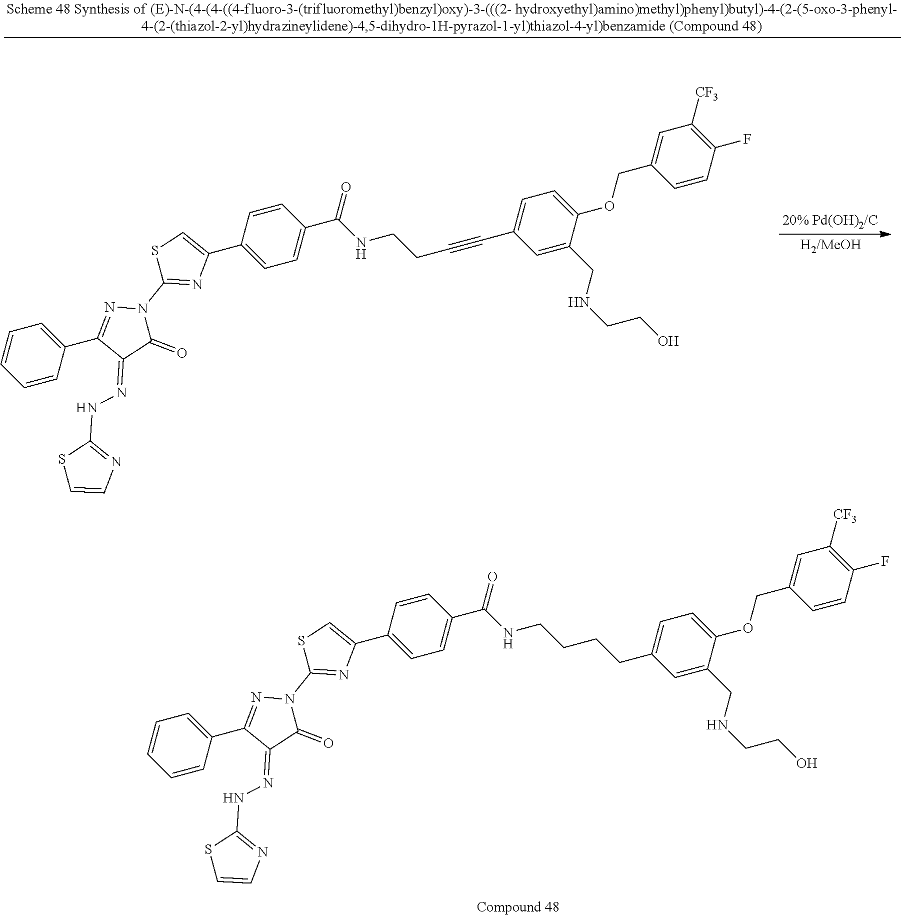 [SMILES: O=C(NCCC#Cc1ccc(OCc2ccc(F)c(C(F)(F)F)c2)c(CNCCO)c1)c1ccc(-c2csc(N3N=C(c4ccccc4)/C(=N\Nc4nccs4)C3=O)n2)cc1 ; O=C(NCCCCc1ccc(OCc2ccc(F)c(C(F)(F)F)c2)c(CNCCO)c1)c1ccc(-c2csc(N3N=C(c4ccccc4)/C(=N\Nc4nccs4)C3=O)n2)cc1]

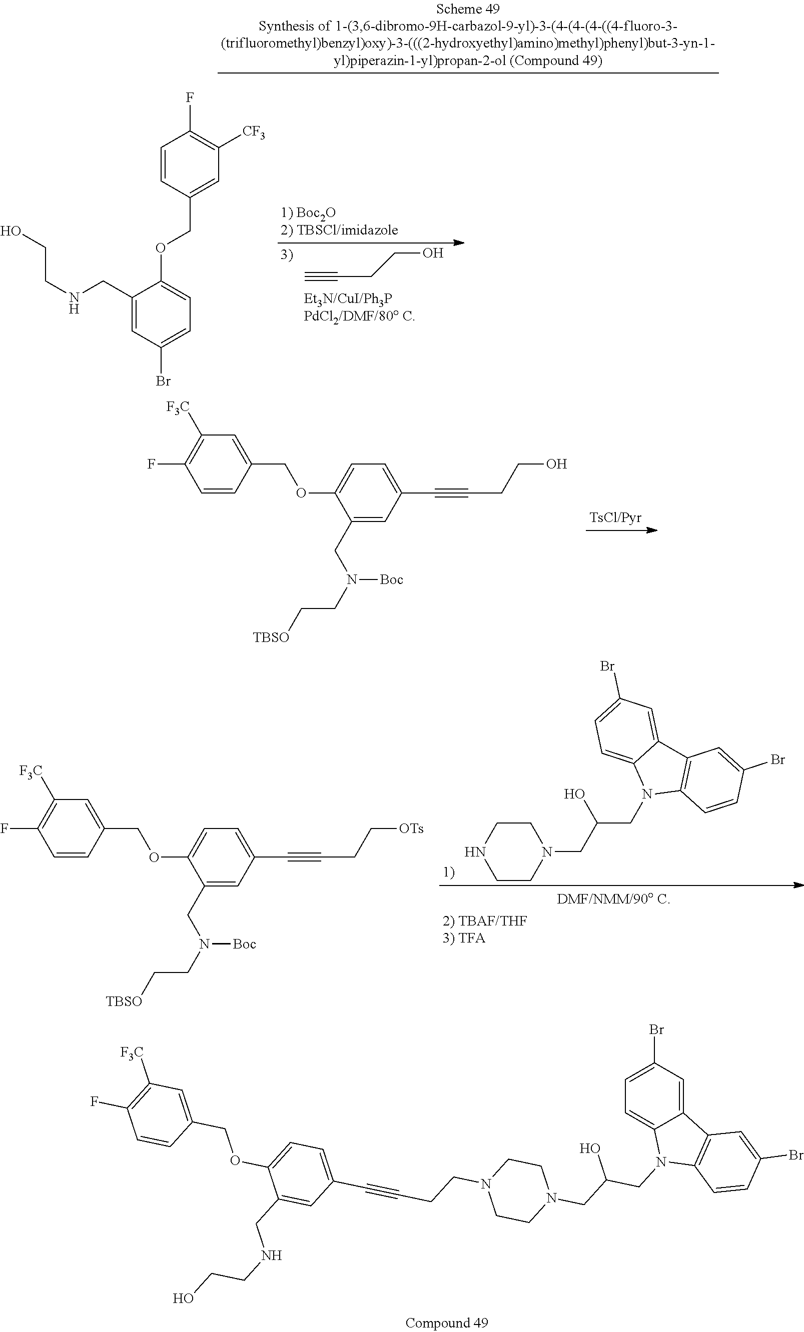

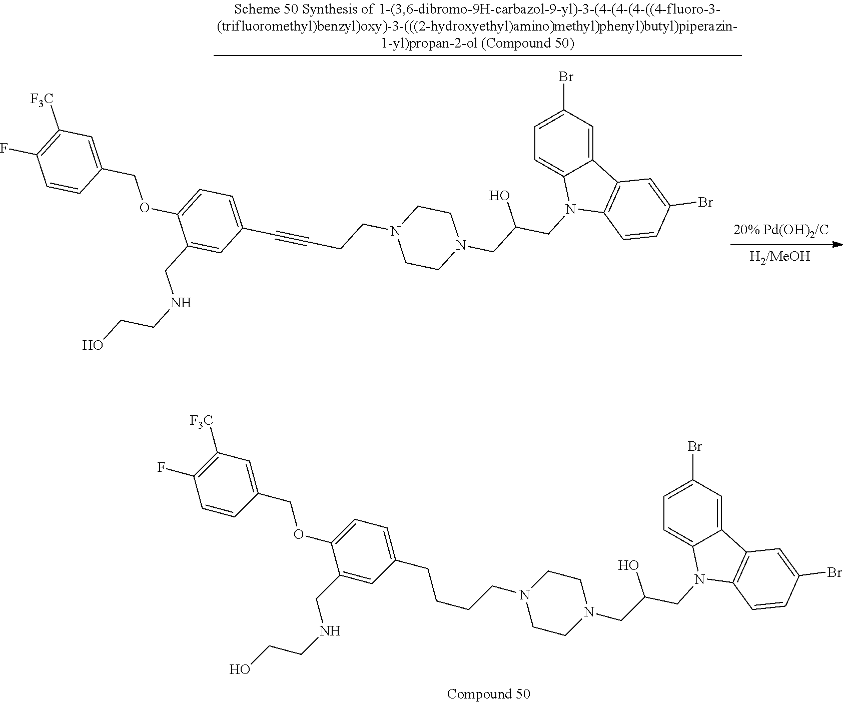 [SMILES: OCCNCc1cc(C#CCCN2CCN(CC(O)Cn3c4ccc(Br)cc4c4cc(Br)ccc43)CC2)ccc1OCc1ccc(F)c(C(F)(F)F)c1 ; OCCNCc1cc(CCCCN2CCN(CC(O)Cn3c4ccc(Br)cc4c4cc(Br)ccc43)CC2)ccc1OCc1ccc(F)c(C(F)(F)F)c1]

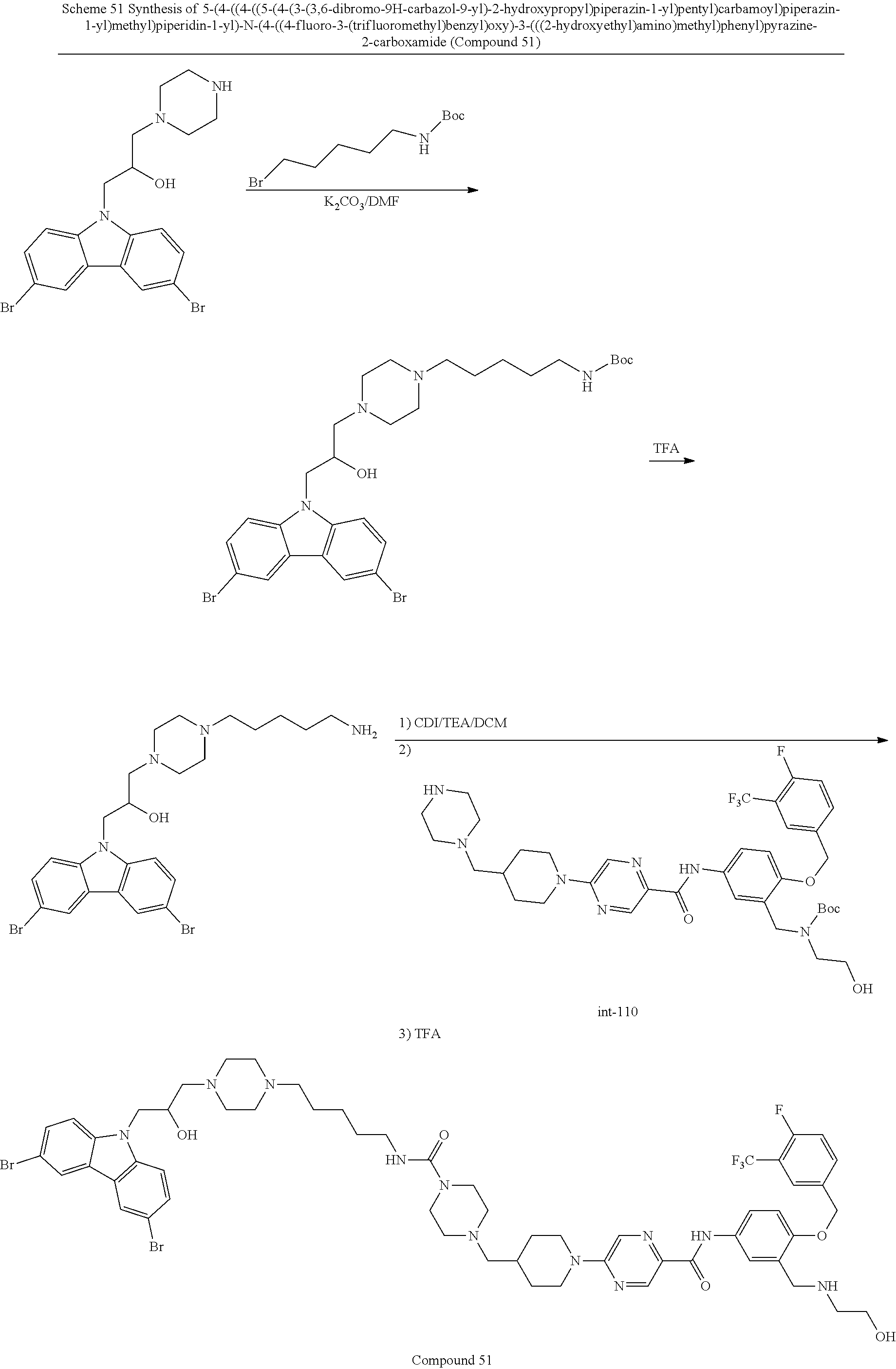 [SMILES: OC(CN1CCNCC1)Cn1c2ccc(Br)cc2c2cc(Br)ccc21 ; CC(C)(C)OC(=O)NCCCCCBr ; CC(C)(C)OC(=O)NCCCCCN1CCN(CC(O)Cn2c3ccc(Br)cc3c3cc(Br)ccc32)CC1 ; NCCCCCN1CCN(CC(O)Cn2c3ccc(Br)cc3c3cc(Br)ccc32)CC1 ; CC(C)(C)OC(=O)N(CCO)Cc1cc(NC(=O)c2cnc(N3CCC(CN4CCNCC4)CC3)cn2)ccc1OCc1ccc(F)c(C(F)(F)F)c1 ; O=C(Nc1ccc(OCc2ccc(F)c(C(F)(F)F)c2)c(CNCCO)c1)c1cnc(N2CCC(CN3CCN(C(=O)NCCCCCN4CCN(CC(O)Cn5c6ccc(Br)cc6c6cc(Br)ccc65)CC4)CC3)CC2)cn1]

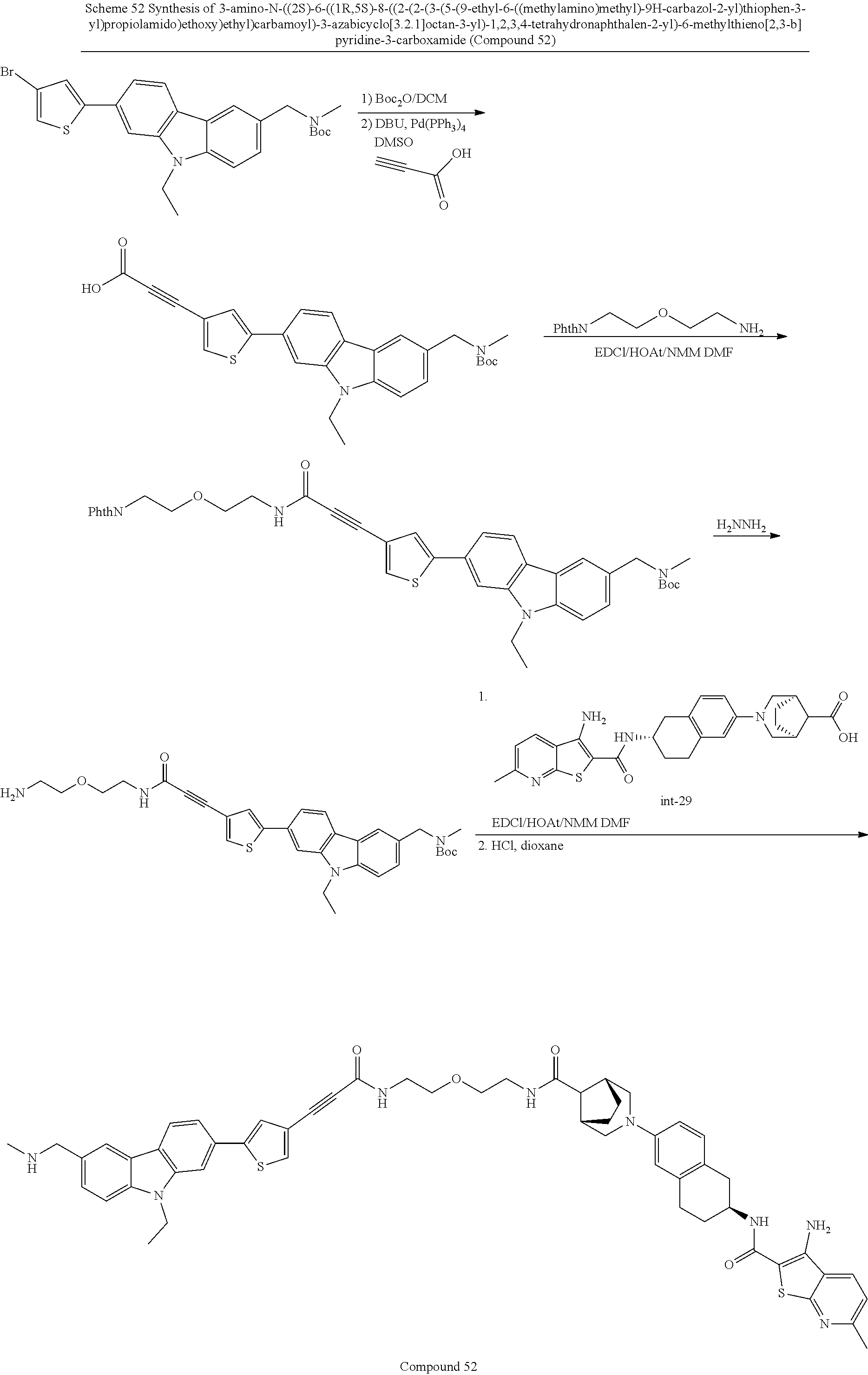

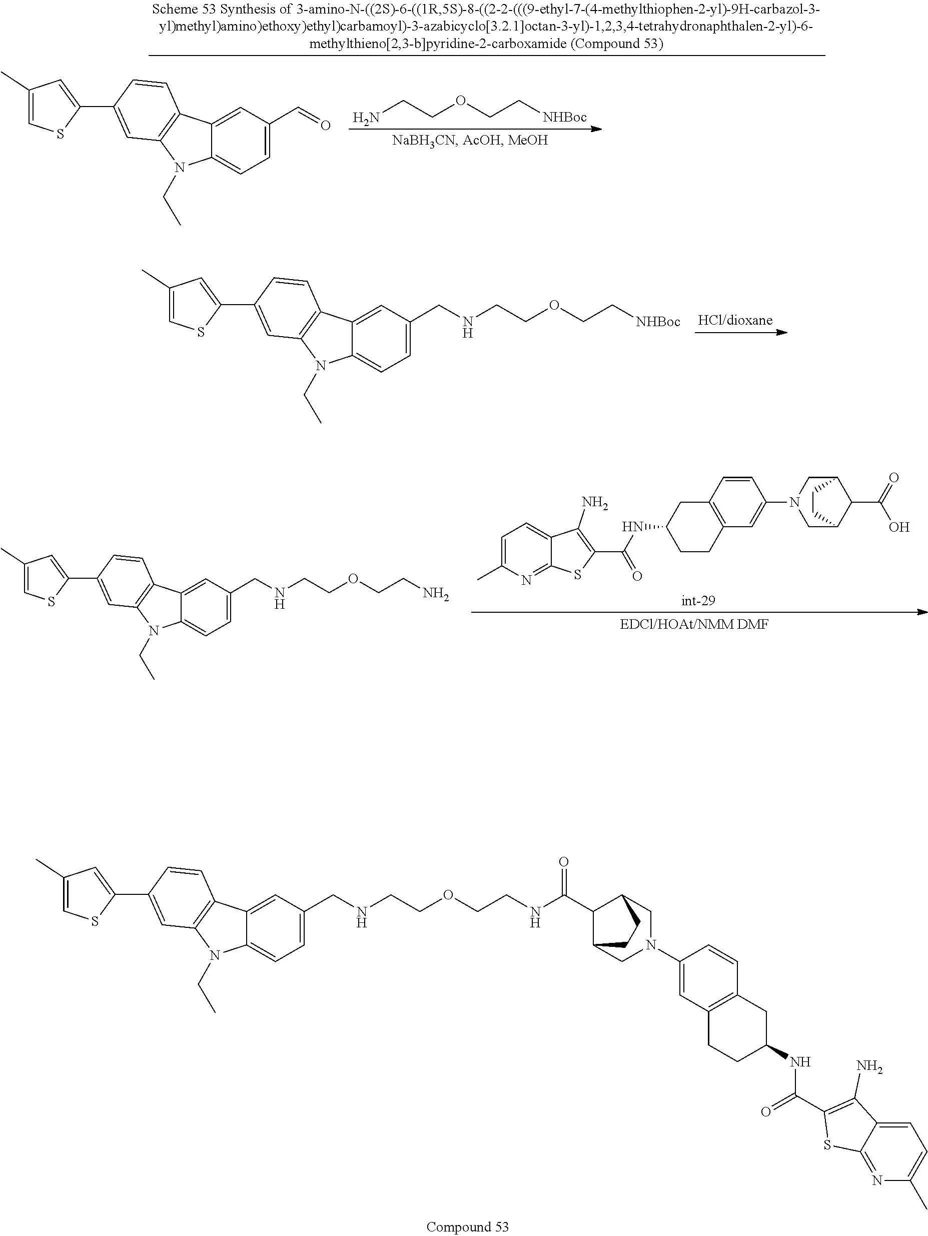

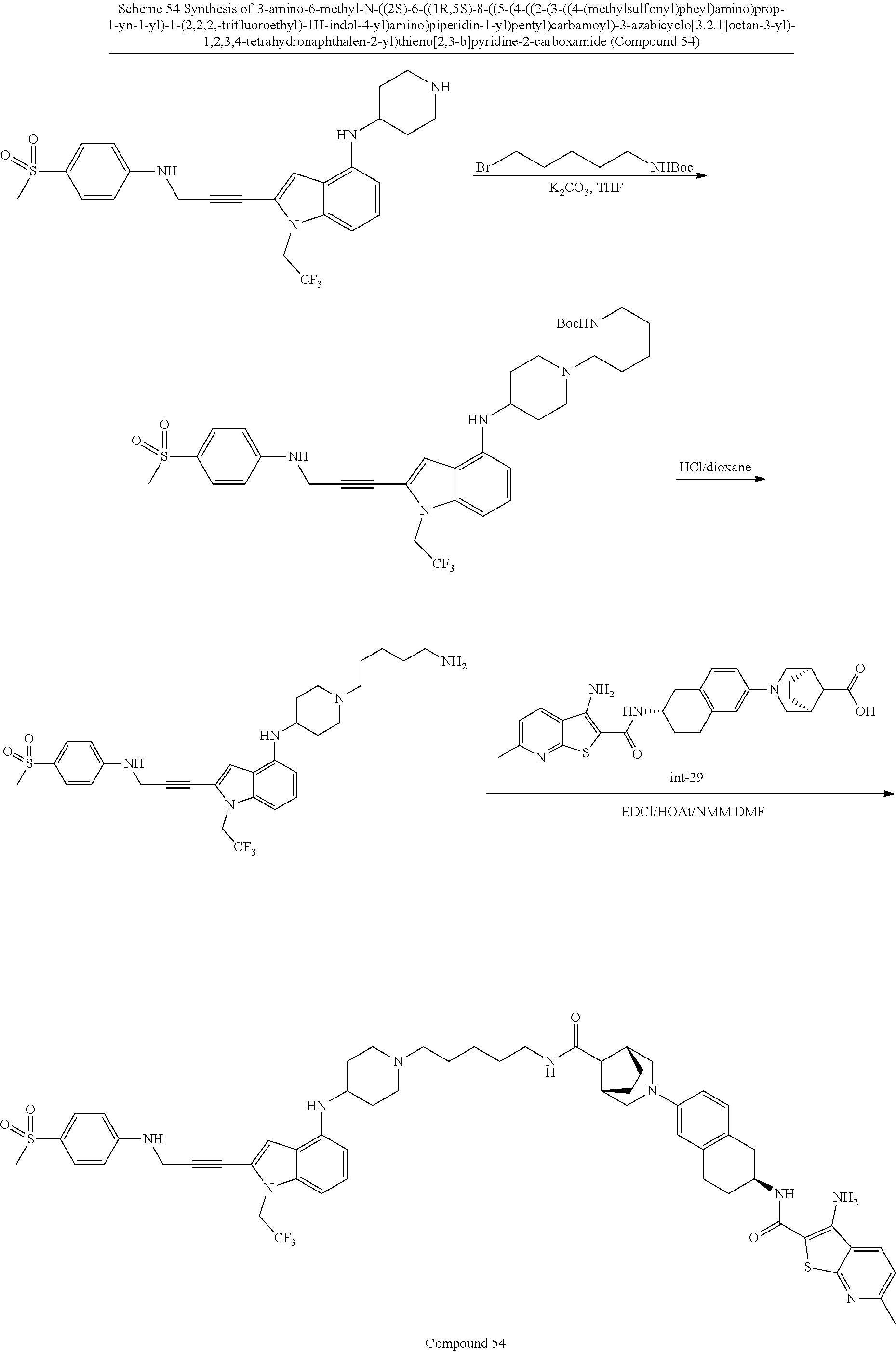 [SMILES: CS(=O)(=O)c1ccc(NCC#Cc2cc3c(NC4CCNCC4)cccc3n2CC(F)(F)F)cc1 ; CC(C)(C)OC(=O)NCCCCCBr ; CC(C)(C)OC(=O)NCCCCCN1CCC(Nc2cccc3c2cc(C#CCNc2ccc(S(C)(=O)=O)cc2)n3CC(F)(F)F)CC1 ; CS(=O)(=O)c1ccc(NCC#Cc2cc3c(NC4CCN(CCCCCN)CC4)cccc3n2CC(F)(F)F)cc1 ; Cc1ccc2c(N)c(C(=O)N[C@H]3CCc4cc(N5C[C@H]6CC[C@@H](C5)C6C(=O)O)ccc4C3)sc2n1 ; Cc1ccc2c(N)c(C(=O)N[C@H]3CCc4cc(N5C[C@H]6CC[C@@H](C5)C6C(=O)NCCCCCN5CCC(Nc6cccc7c6cc(C#CCNc6ccc(S(C)(=O)=O)cc6)n7CC(F)(F)F)CC5)ccc4C3)sc2n1]

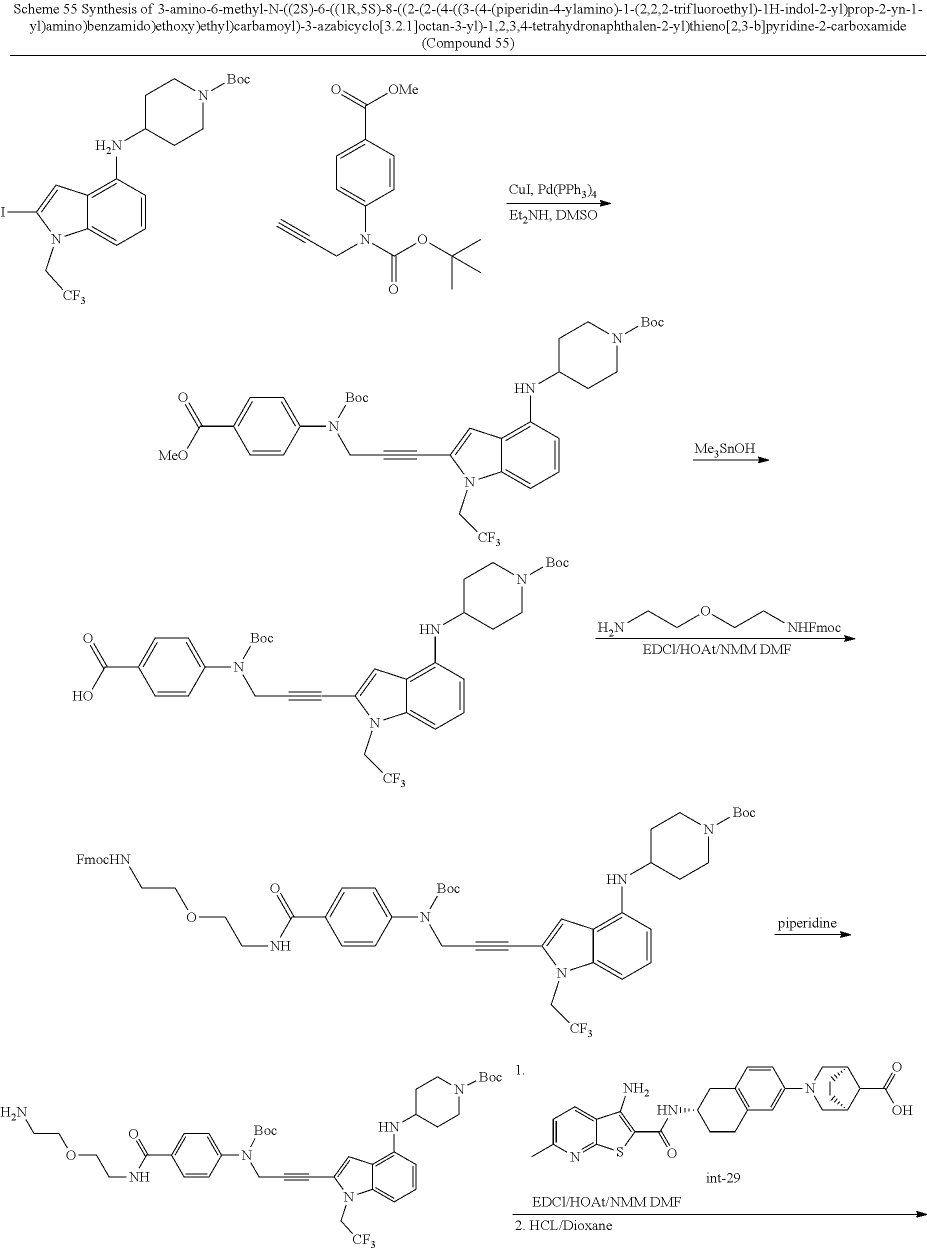

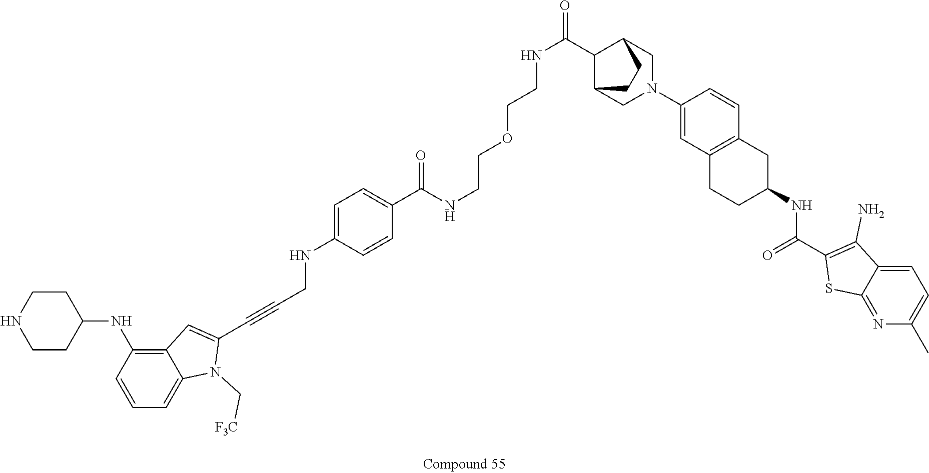 [SMILES: Cc1ccc2c(N)c(C(=O)N[C@H]3CCc4cc(N5C[C@H]6CC[C@@H](C5)C6C(=O)NCCOCCNC(=O)c5ccc(NCC#Cc6cc7c(NC8CCNCC8)cccc7n6CC(F)(F)F)cc5)ccc4C3)sc2n1]

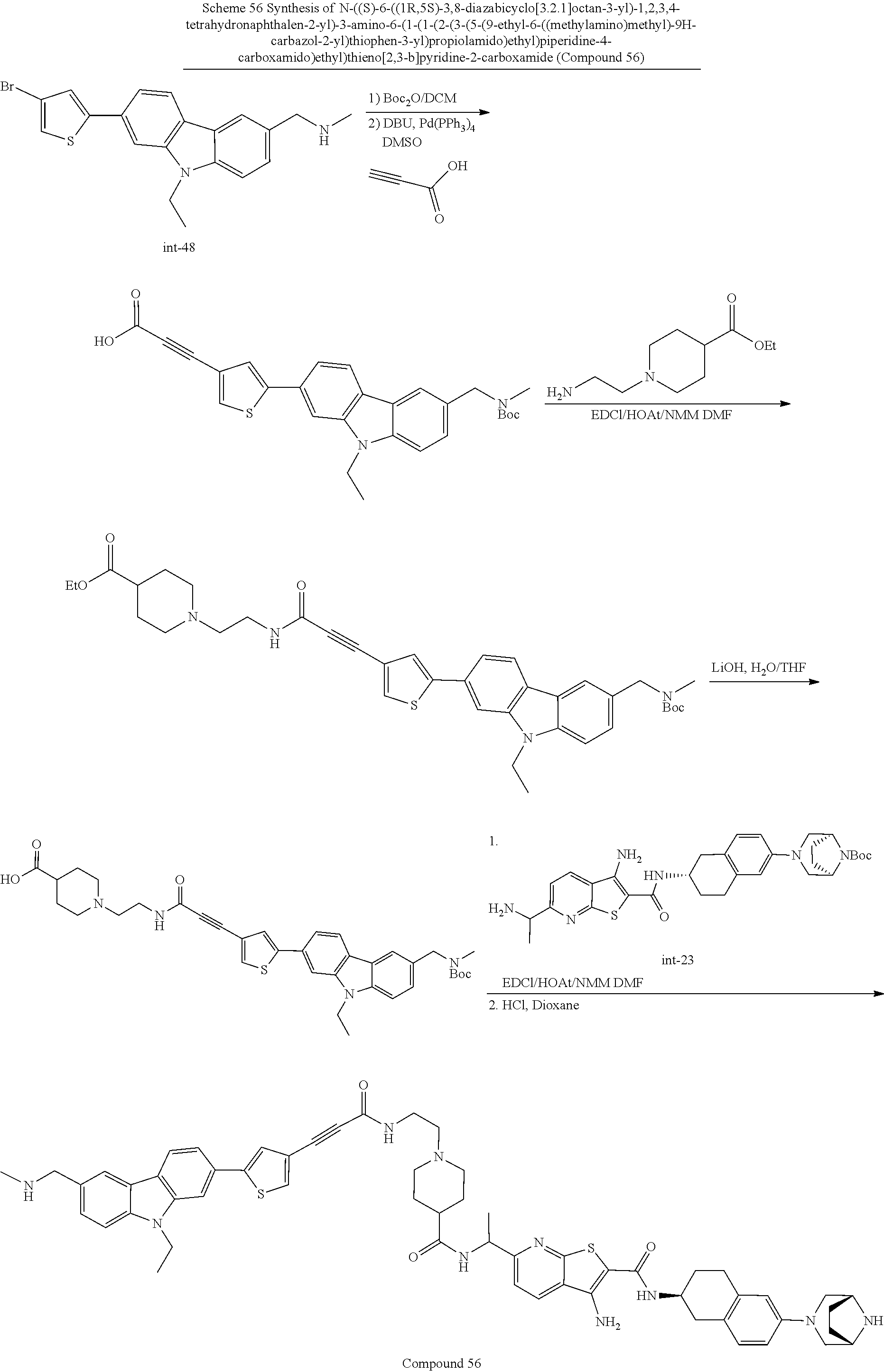

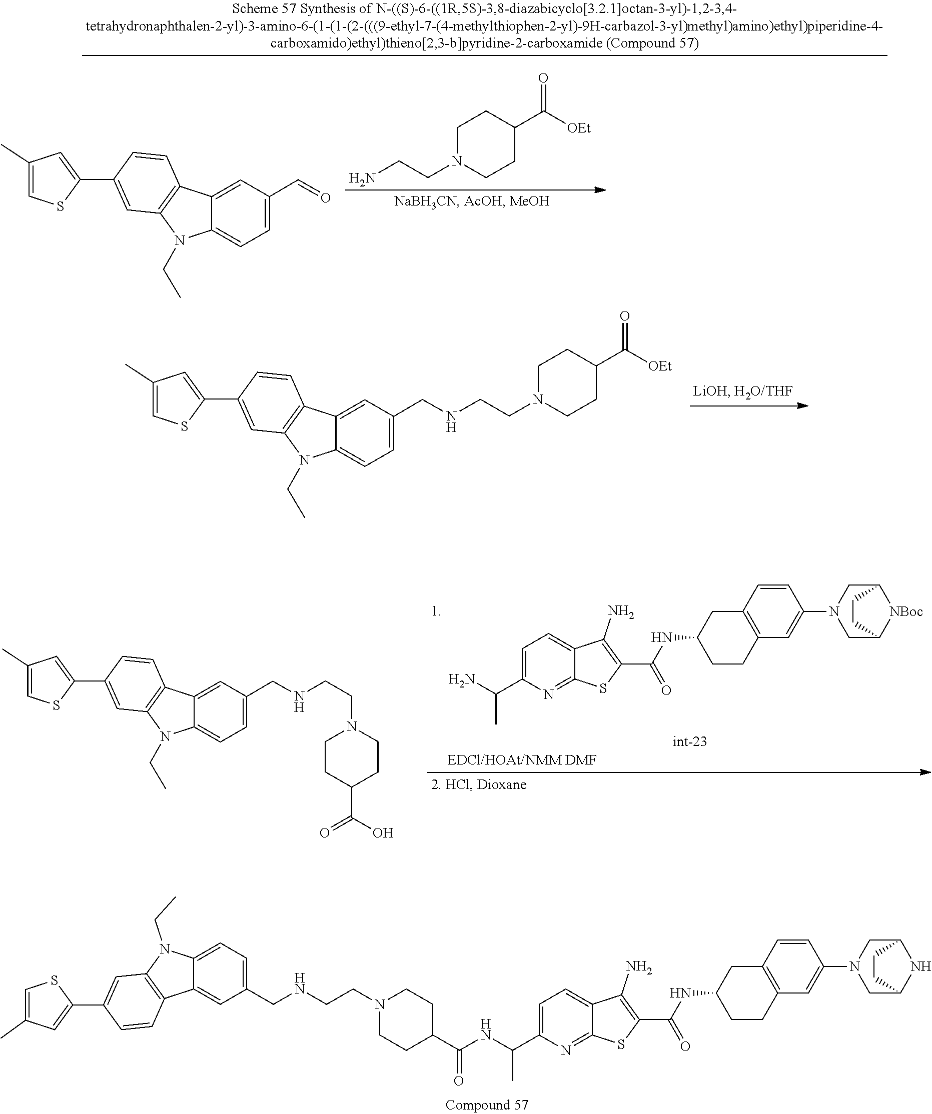 [SMILES: CCn1c2ccc(C=O)cc2c2ccc(-c3cc(C)cs3)cc21 ; CCOC(=O)C1CCN(CCN)CC1 ; CCOC(=O)C1CCN(CCNCc2ccc3c(c2)c2ccc(-c4cc(C)cs4)cc2n3CC)CC1 ; CCn1c2ccc(CNCCN3CCC(C(=O)O)CC3)cc2c2ccc(-c3cc(C)cs3)cc21 ; CC(N)c1ccc2c(N)c(C(=O)N[C@H]3CCc4cc(N5C[C@H]6CC[C@@H](C5)N6C(=O)OC(C)(C)C)ccc4C3)sc2n1 ; CCn1c2ccc(CNCCN3CCC(C(=O)NC(C)c4ccc5c(N)c(C(=O)N[C@H]6CCc7cc(N8C[C@H]9CC[C@@H](C8)N9)ccc7C6)sc5n4)CC3)cc2c2ccc(-c3cc(C)cs3)cc21]

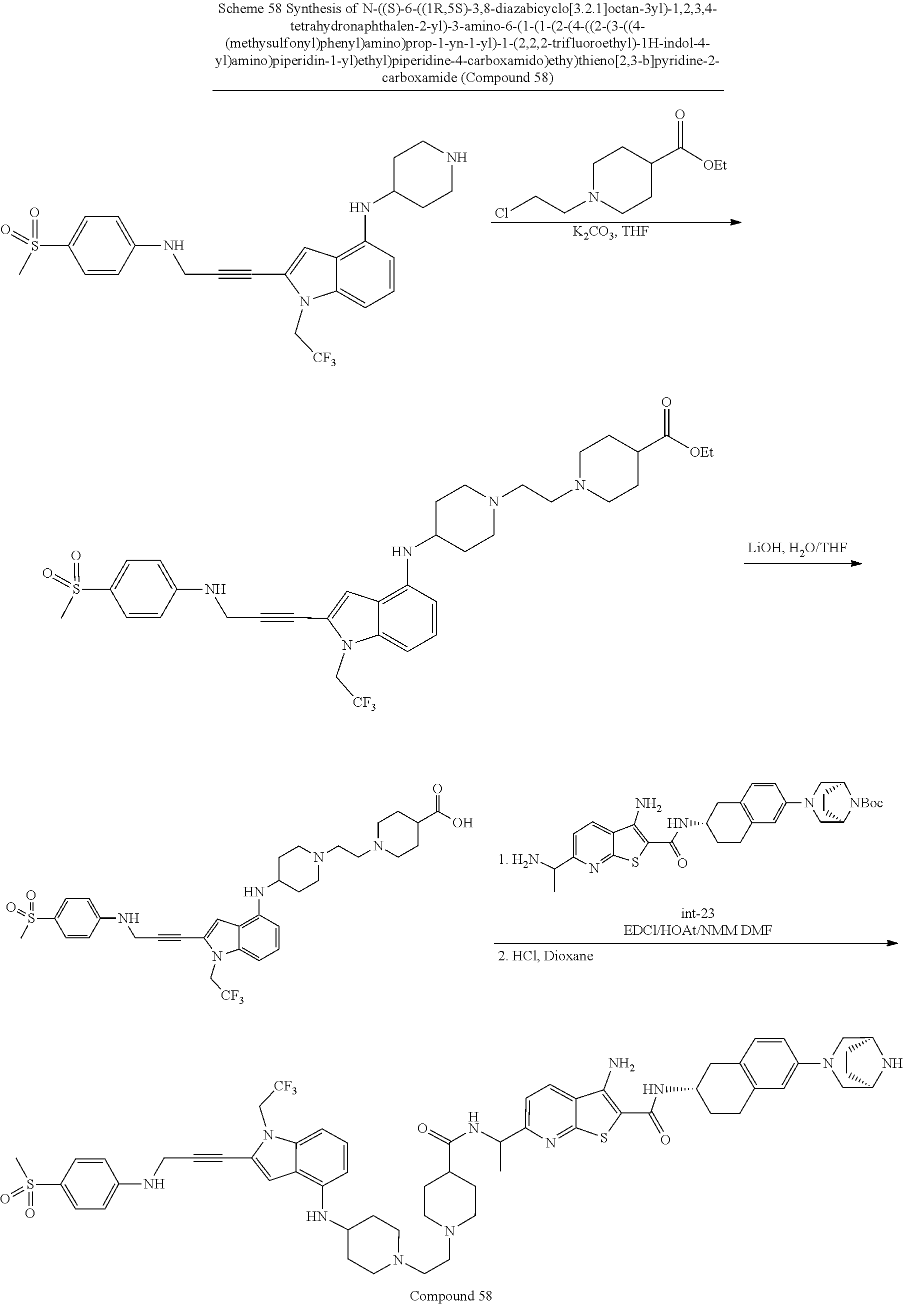

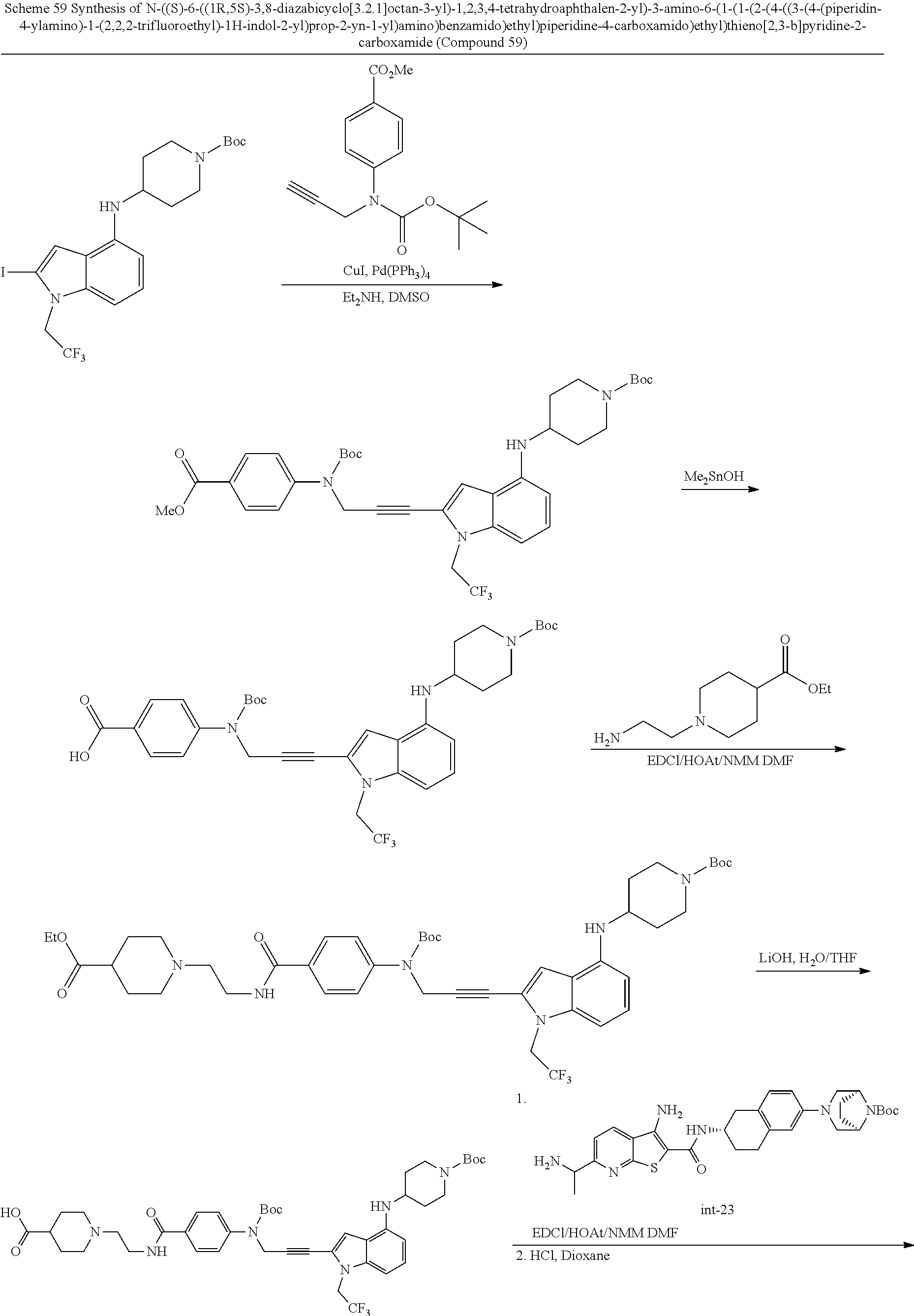

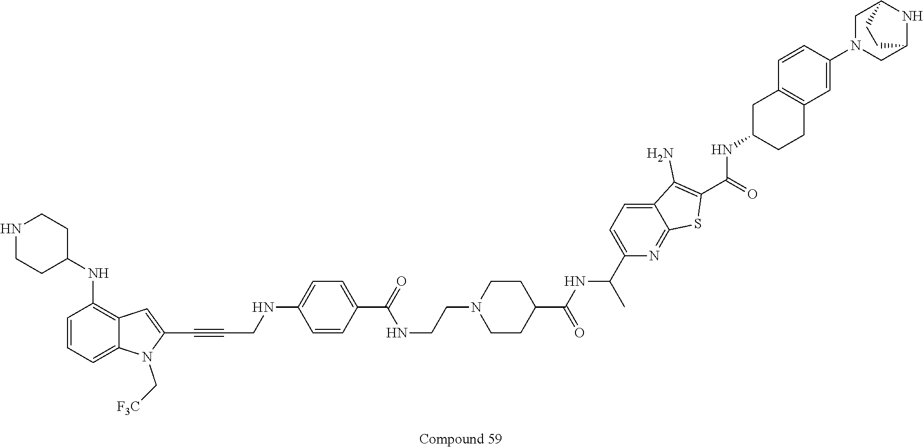 [SMILES: CC(NC(=O)C1CCN(CCNC(=O)c2ccc(NCC#Cc3cc4c(NC5CCNCC5)cccc4n3CC(F)(F)F)cc2)CC1)c1ccc2c(N)c(C(=O)N[C@H]3CCc4cc(N5C[C@H]6CC[C@@H](C5)N6)ccc4C3)sc2n1]

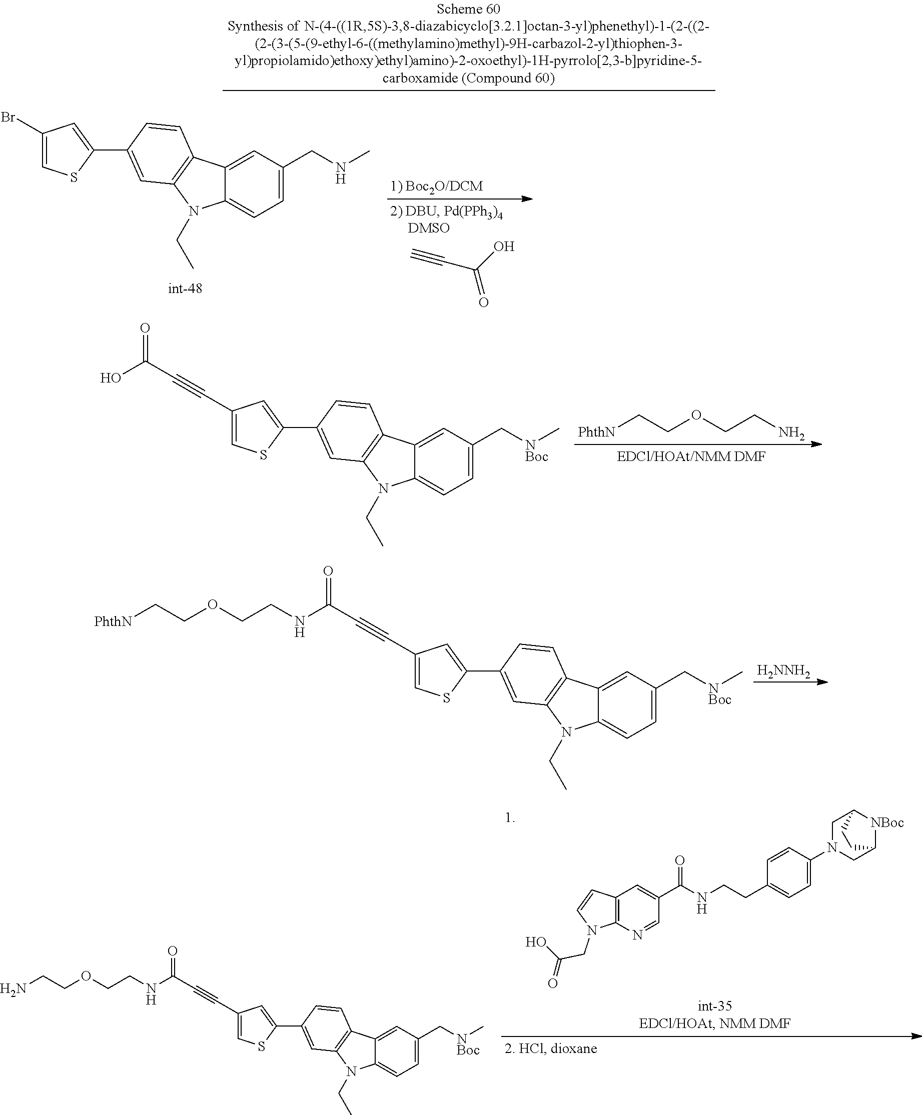

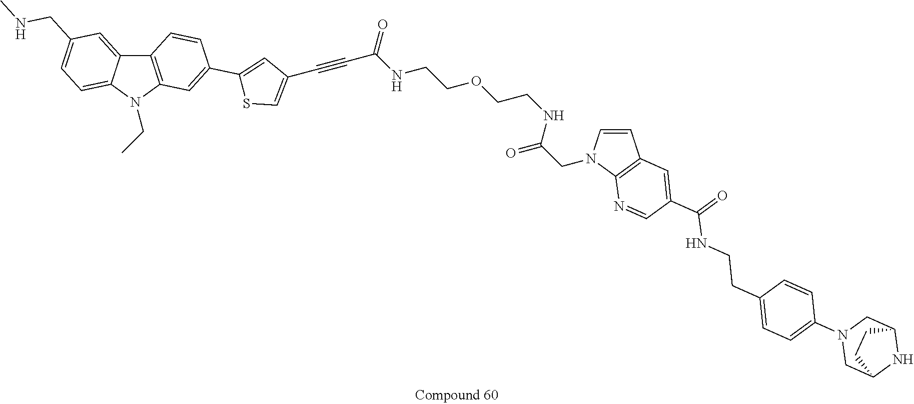 [SMILES: CCn1c2ccc(CNC)cc2c2ccc(-c3cc(C#CC(=O)NCCOCCNC(=O)Cn4ccc5cc(C(=O)NCCc6ccc(N7C[C@H]8CC[C@@H](C7)N8)cc6)cnc54)cs3)cc21]

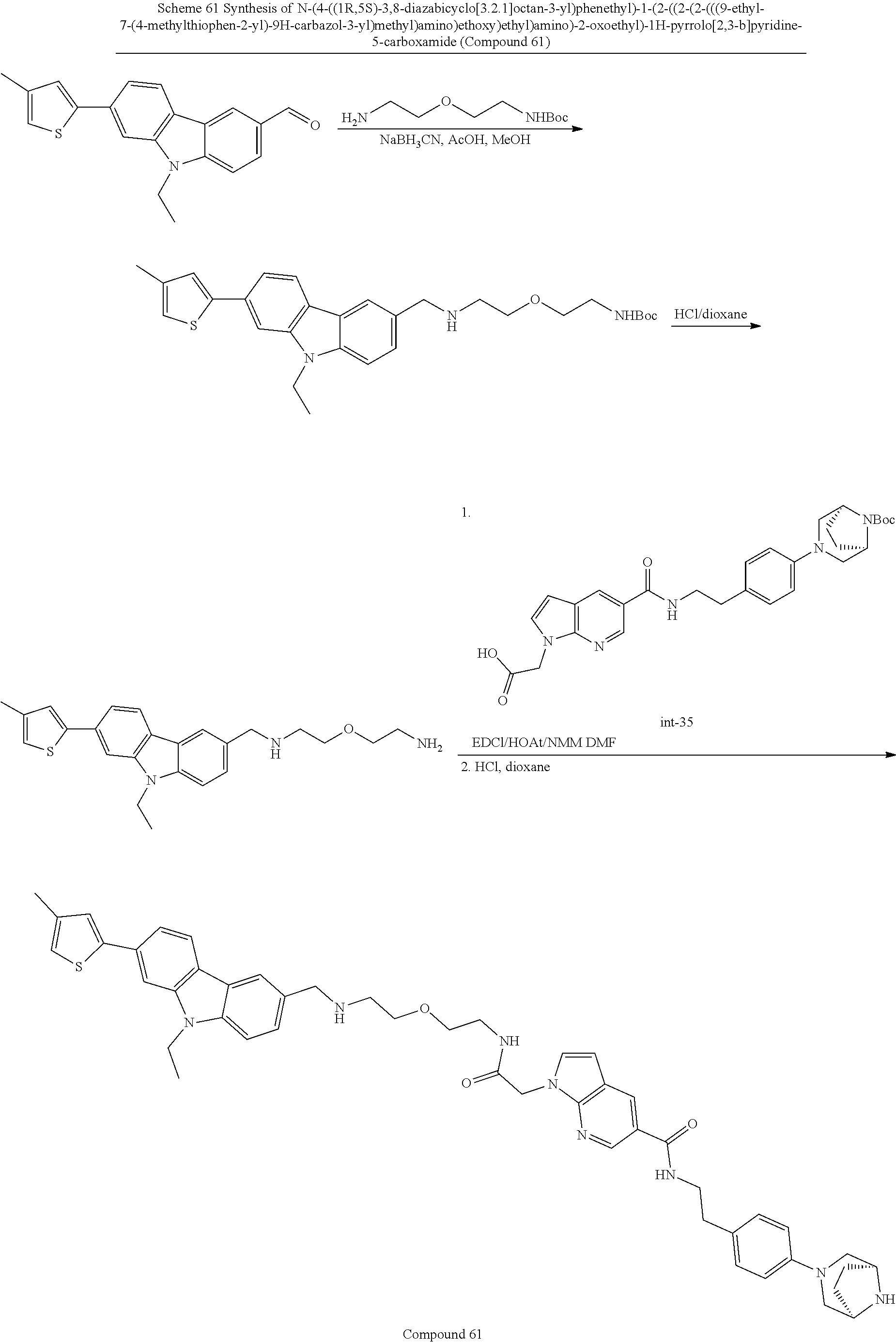 [SMILES: CCn1c2ccc(C=O)cc2c2ccc(-c3cc(C)cs3)cc21 ; CC(C)(C)OC(=O)NCCOCCN ; CCn1c2ccc(CNCCOCCNC(=O)OC(C)(C)C)cc2c2ccc(-c3cc(C)cs3)cc21 ; CC(C)(C)OC(=O)N1[C@@H]2CC[C@H]1CN(c1ccc(CCNC(=O)c3cnc4c(ccn4CC(=O)O)c3)cc1)C2 ; CCn1c2ccc(CNCCOCCN)cc2c2ccc(-c3cc(C)cs3)cc21 ; CCn1c2ccc(CNCCOCCNC(=O)Cn3ccc4cc(C(=O)NCCc5ccc(N6C[C@H]7CC[C@@H](C6)N7)cc5)cnc43)cc2c2ccc(-c3cc(C)cs3)cc21]

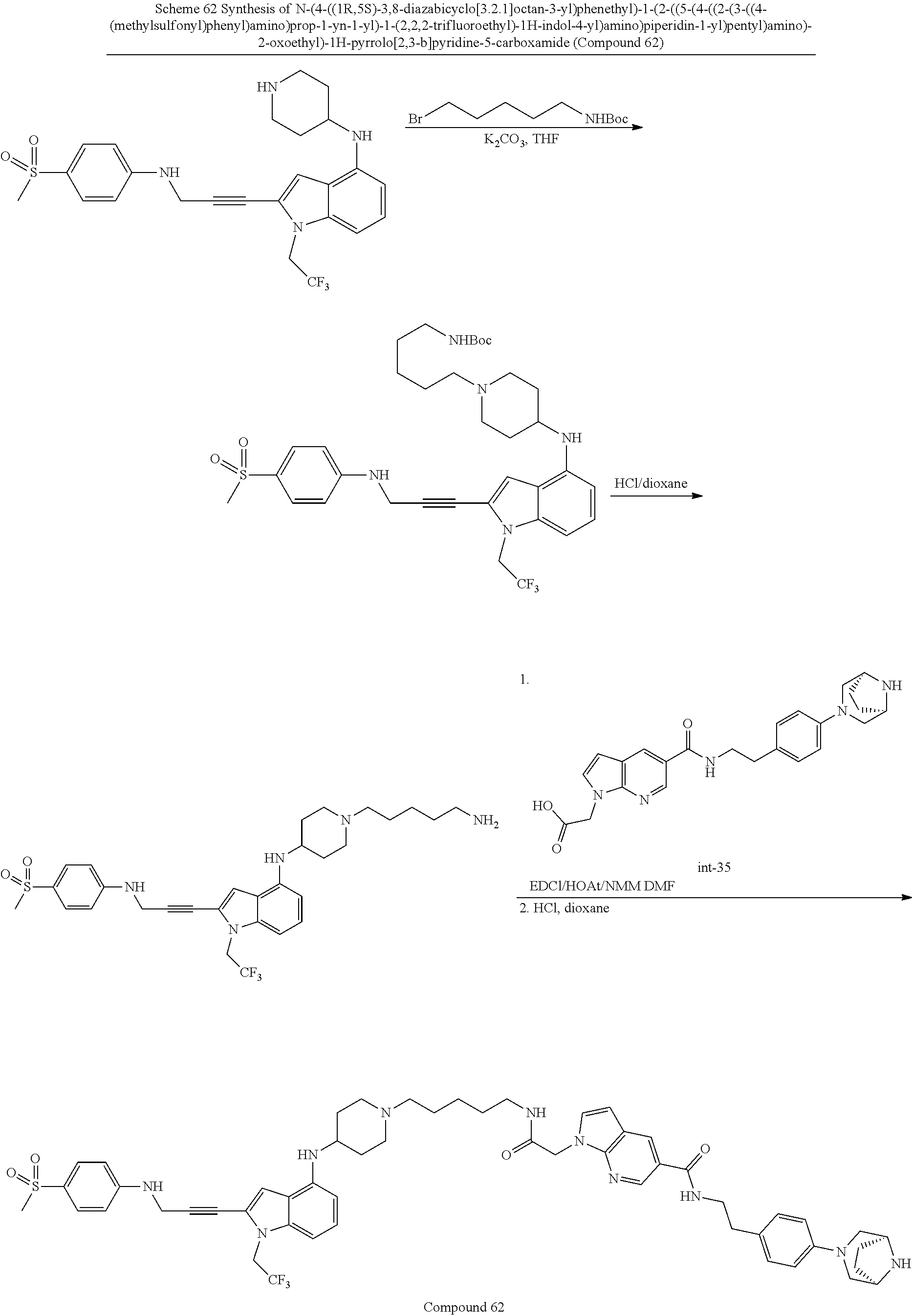 [SMILES: CS(=O)(=O)c1ccc(NCC#Cc2cc3c(NC4CCNCC4)cccc3n2CC(F)(F)F)cc1 ; CC(C)(C)OC(=O)NCCCCCBr ; CC(C)(C)OC(=O)NCCCCCN1CCC(Nc2cccc3c2cc(C#CCNc2ccc(S(C)(=O)=O)cc2)n3CC(F)(F)F)CC1 ; CS(=O)(=O)c1ccc(NCC#Cc2cc3c(NC4CCN(CCCCCN)CC4)cccc3n2CC(F)(F)F)cc1 ; O=C(O)Cn1ccc2cc(C(=O)NCCc3ccc(N4C[C@H]5CC[C@@H](C4)N5)cc3)cnc21 ; CS(=O)(=O)c1ccc(NCC#Cc2cc3c(NC4CCN(CCCCCNC(=O)Cn5ccc6cc(C(=O)NCCc7ccc(N8C[C@H]9CC[C@@H](C8)N9)cc7)cnc65)CC4)cccc3n2CC(F)(F)F)cc1]

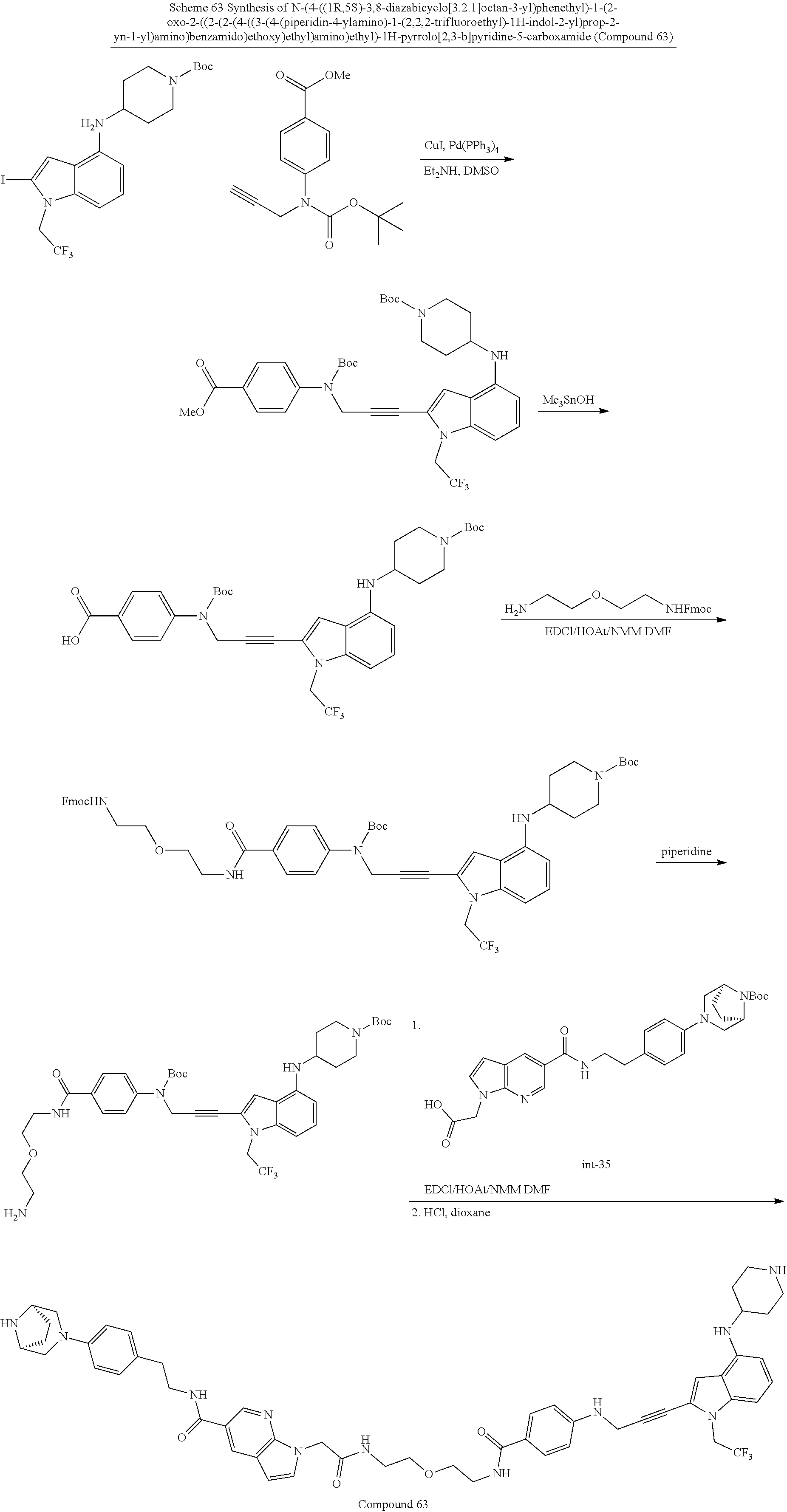

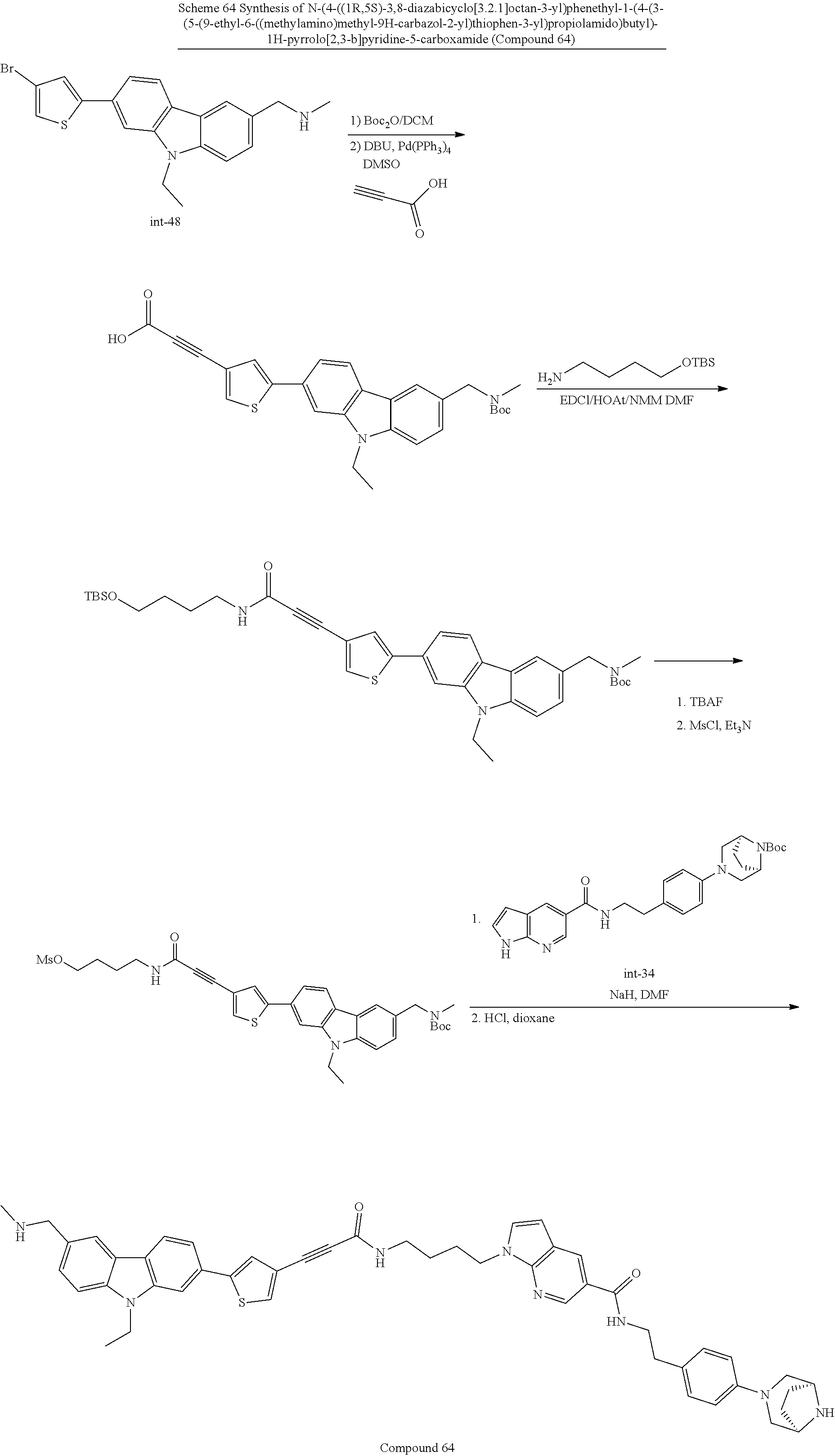

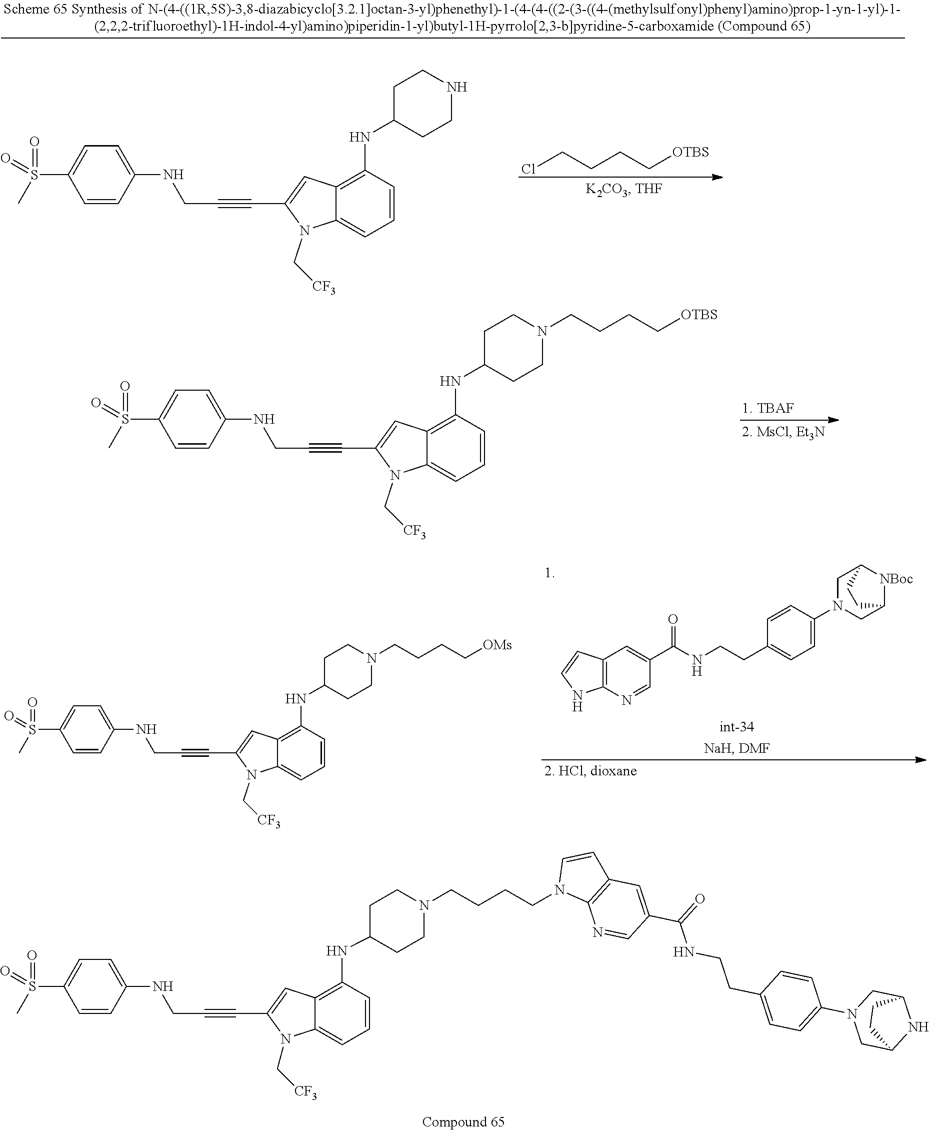 [SMILES: CS(=O)(=O)c1ccc(NCC#Cc2cc3c(NC4CCNCC4)cccc3n2CC(F)(F)F)cc1 ; CC(C)(C)[Si](C)(C)OCCCCCl ; CC(C)(C)[Si](C)(C)OCCCCN1CCC(Nc2cccc3c2cc(C#CCNc2ccc(S(C)(=O)=O)cc2)n3CC(F)(F)F)CC1 ; CS(=O)(=O)OCCCCN1CCC(Nc2cccc3c2cc(C#CCNc2ccc(S(C)(=O)=O)cc2)n3CC(F)(F)F)CC1 ; CC(C)(C)OC(=O)N1[C@@H]2CC[C@H]1CN(c1ccc(CCNC(=O)c3cnc4[nH]ccc4c3)cc1)C2 ; CS(=O)(=O)c1ccc(NCC#Cc2cc3c(NC4CCN(CCCCn5ccc6cc(C(=O)NCCc7ccc(N8C[C@H]9CC[C@@H](C8)N9)cc7)cnc65)CC4)cccc3n2CC(F)(F)F)cc1]

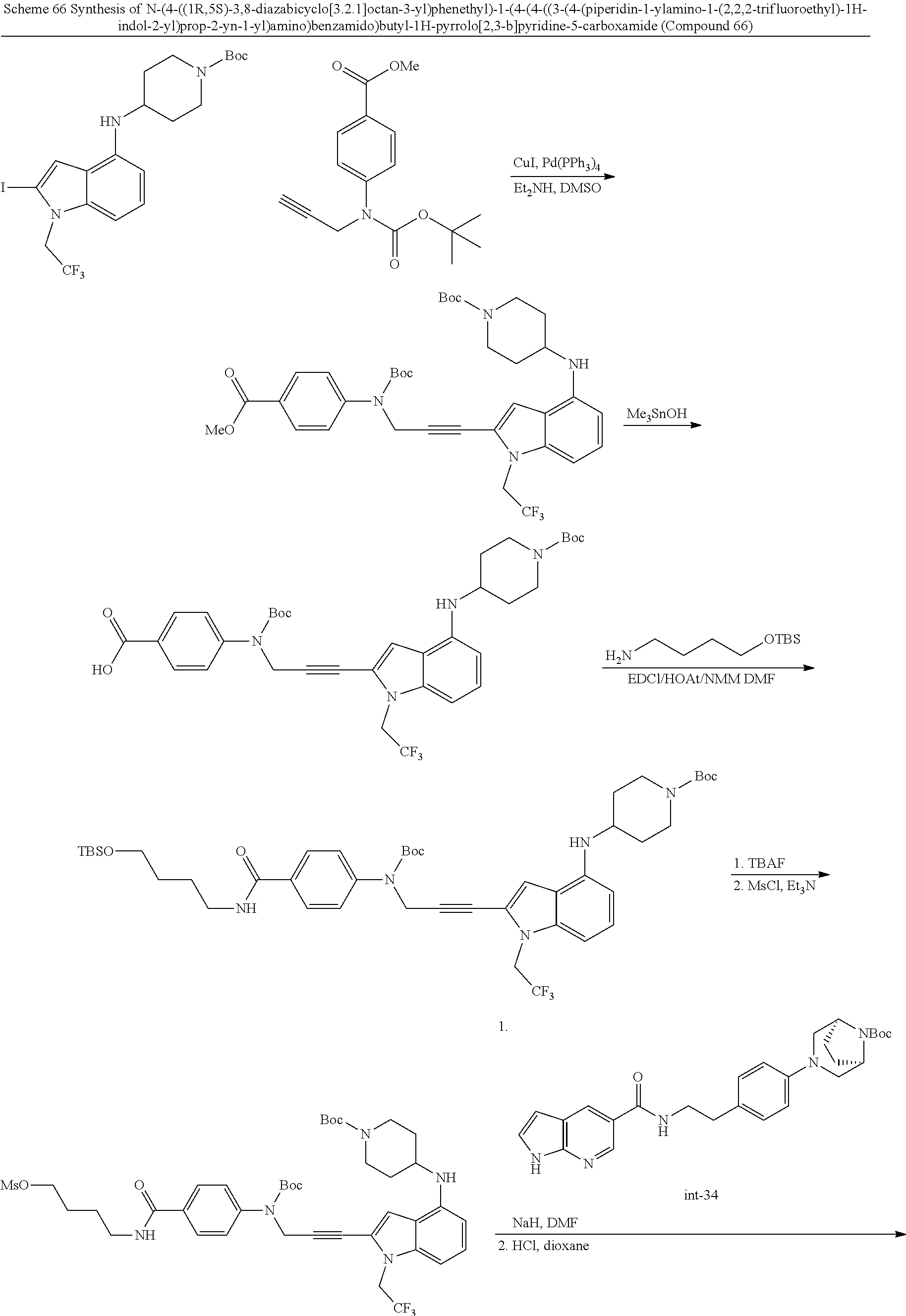

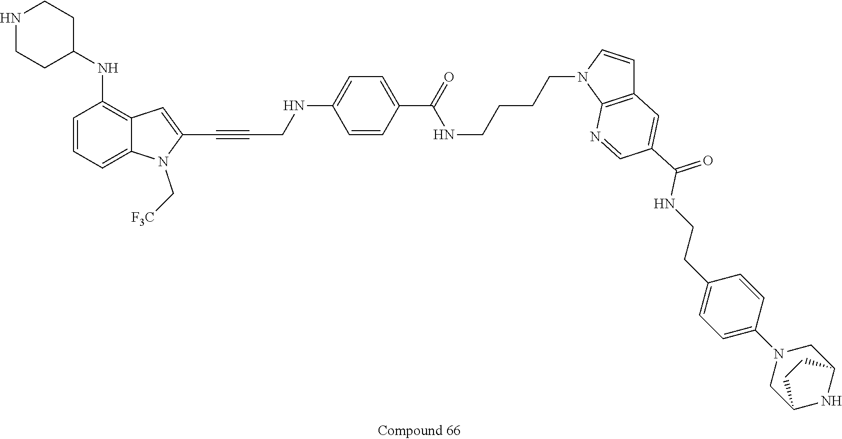 [SMILES: O=C(NCCCCn1ccc2cc(C(=O)NCCc3ccc(N4C[C@H]5CC[C@@H](C4)N5)cc3)cnc21)c1ccc(NCC#Cc2cc3c(NC4CCNCC4)cccc3n2CC(F)(F)F)cc1]

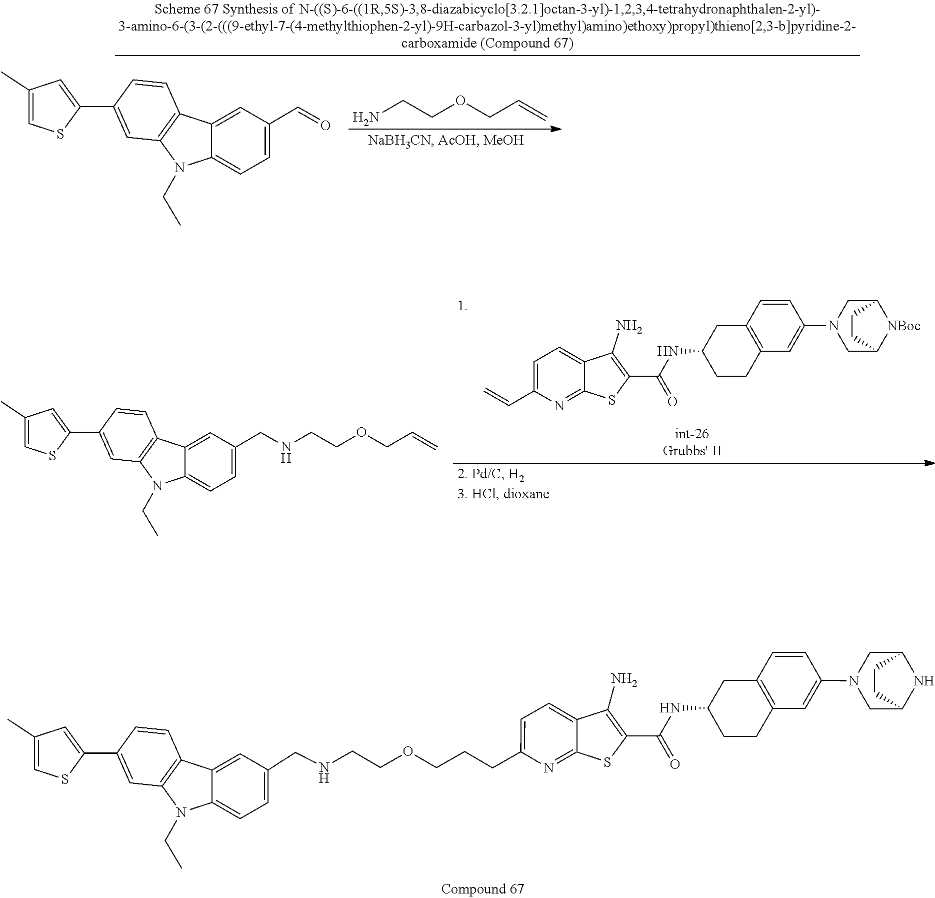

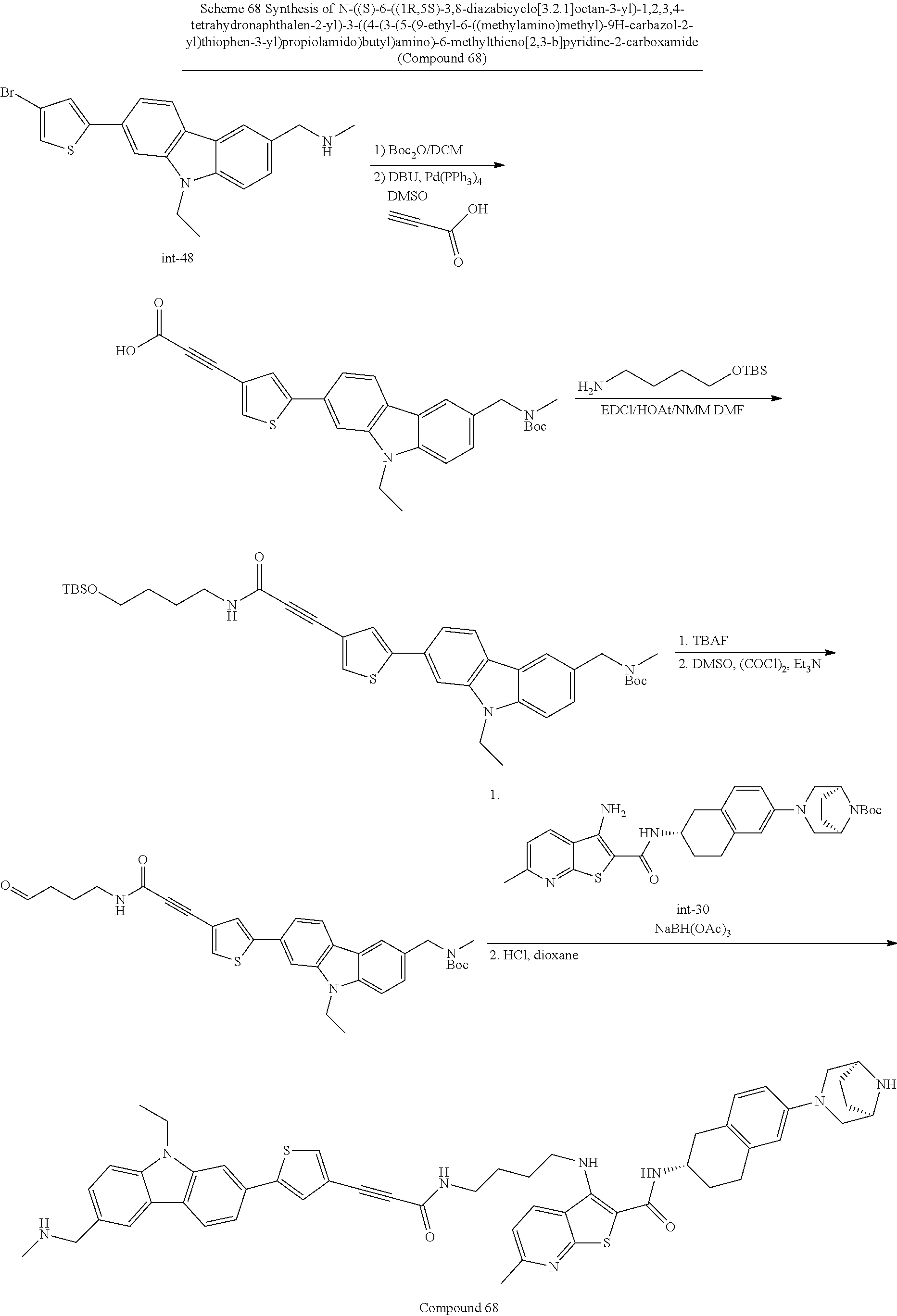

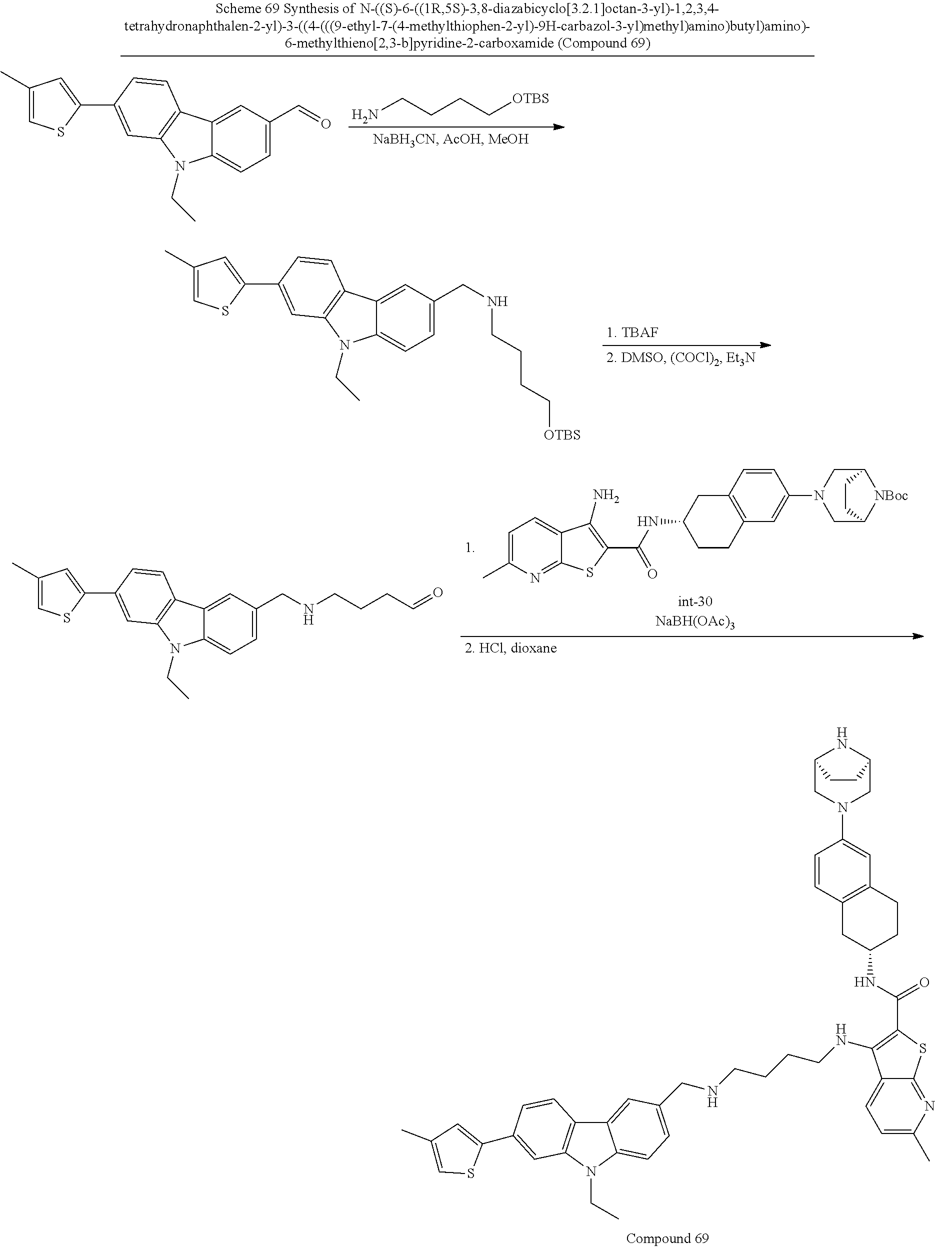

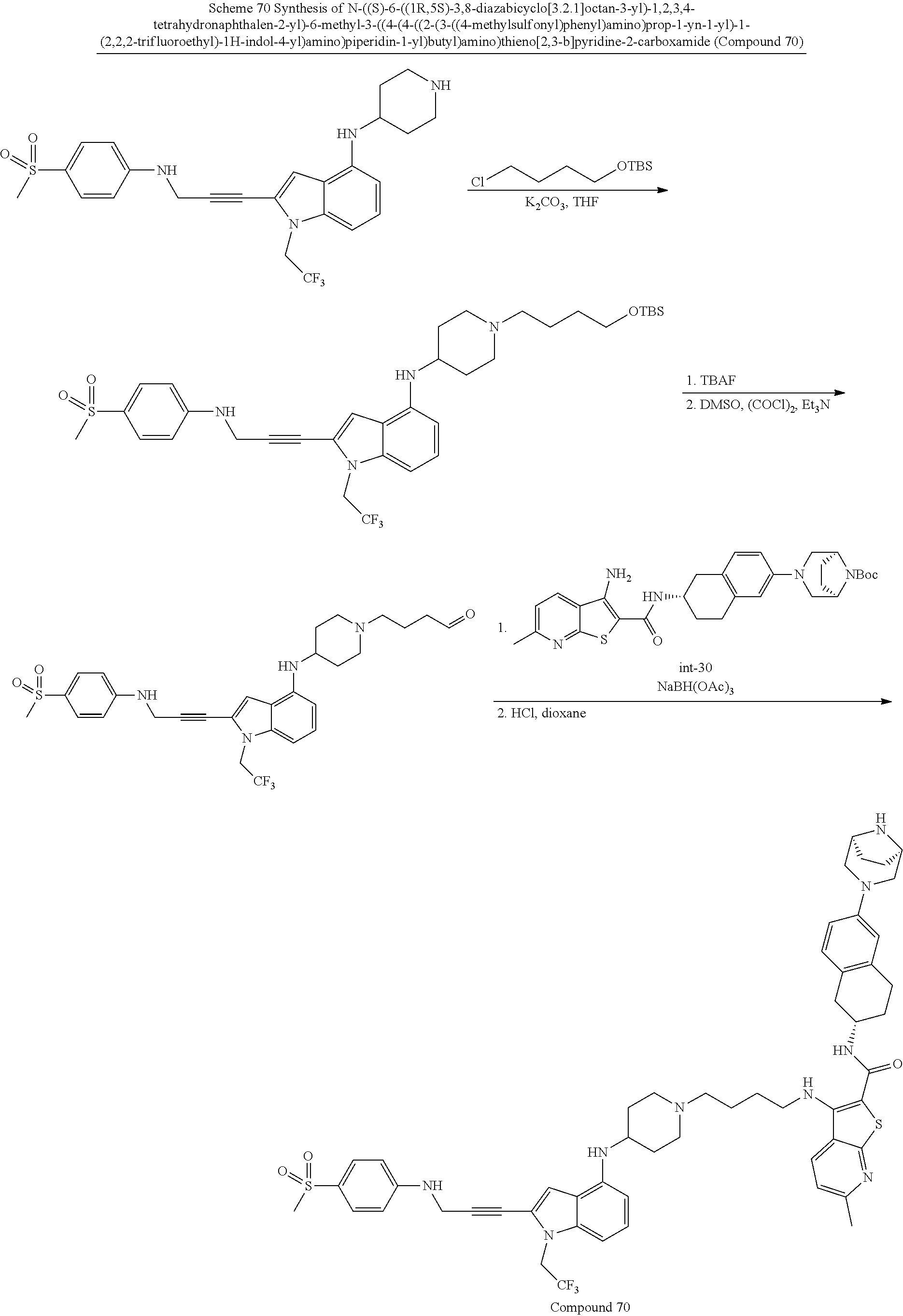 [SMILES: CS(=O)(=O)c1ccc(NCC#Cc2cc3c(NC4CCNCC4)cccc3n2CC(F)(F)F)cc1 ; CC(C)(C)[Si](C)(C)OCCCCCl ; CC(C)(C)[Si](C)(C)OCCCCN1CCC(Nc2cccc3c2cc(C#CCNc2ccc(S(C)(=O)=O)cc2)n3CC(F)(F)F)CC1 ; CS(=O)(=O)c1ccc(NCC#Cc2cc3c(NC4CCN(CCCC=O)CC4)cccc3n2CC(F)(F)F)cc1 ; Cc1ccc2c(N)c(C(=O)N[C@H]3CCc4cc(N5C[C@H]6CC[C@@H](C5)N6C(=O)OC(C)(C)C)ccc4C3)sc2n1 ; Cc1ccc2c(NCCCCN3CCC(Nc4cccc5c4cc(C#CCNc4ccc(S(C)(=O)=O)cc4)n5CC(F)(F)F)CC3)c(C(=O)N[C@H]3CCc4cc(N5C[C@H]6CC[C@@H](C5)N6)ccc4C3)sc2n1]

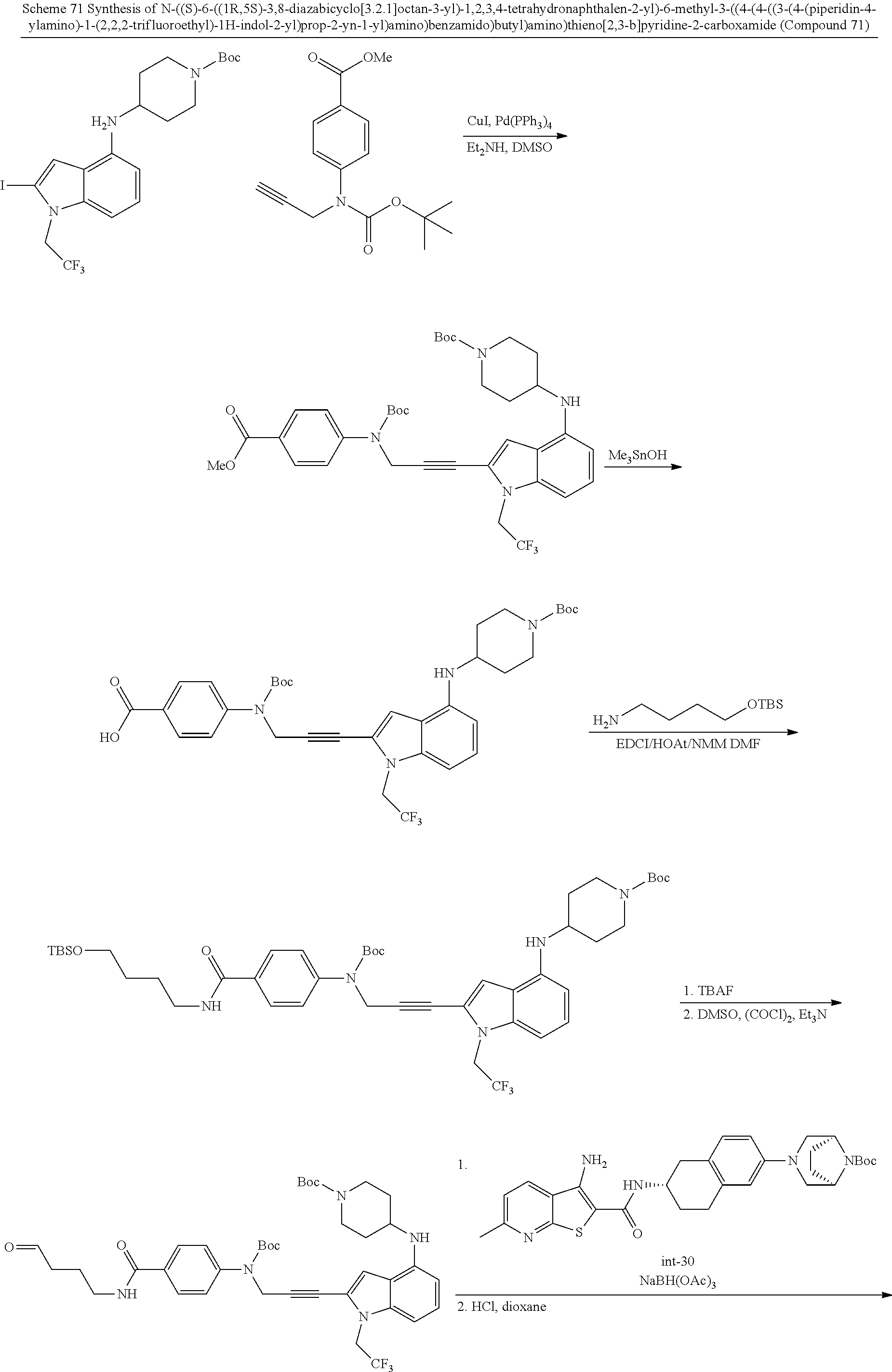

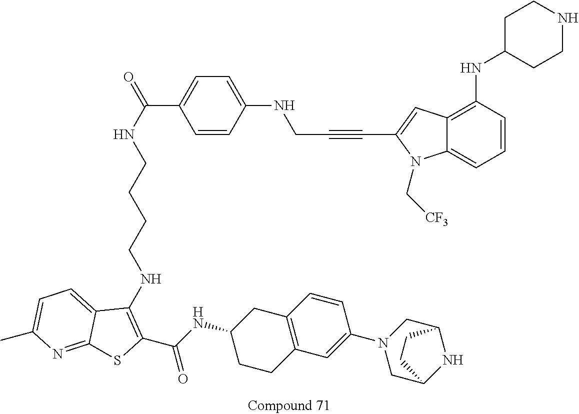 [SMILES: Cc1ccc2c(NCCCCNC(=O)c3ccc(NCC#Cc4cc5c(NC6CCNCC6)cccc5n4CC(F)(F)F)cc3)c(C(=O)N[C@H]3CCc4cc(N5C[C@H]6CC[C@@H](C5)N6)ccc4C3)sc2n1]

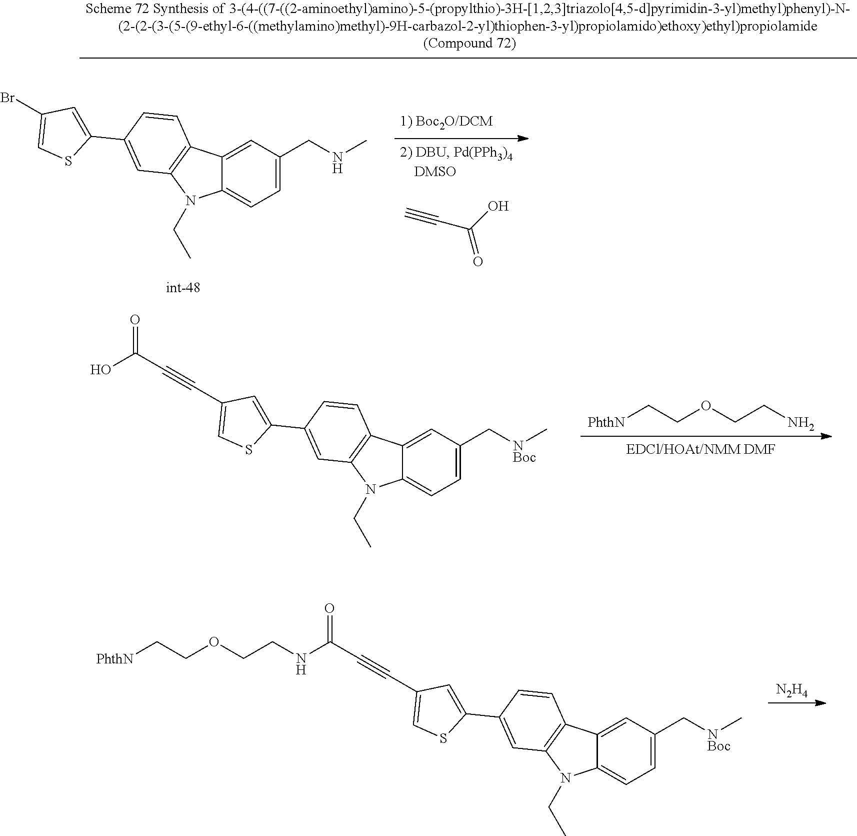

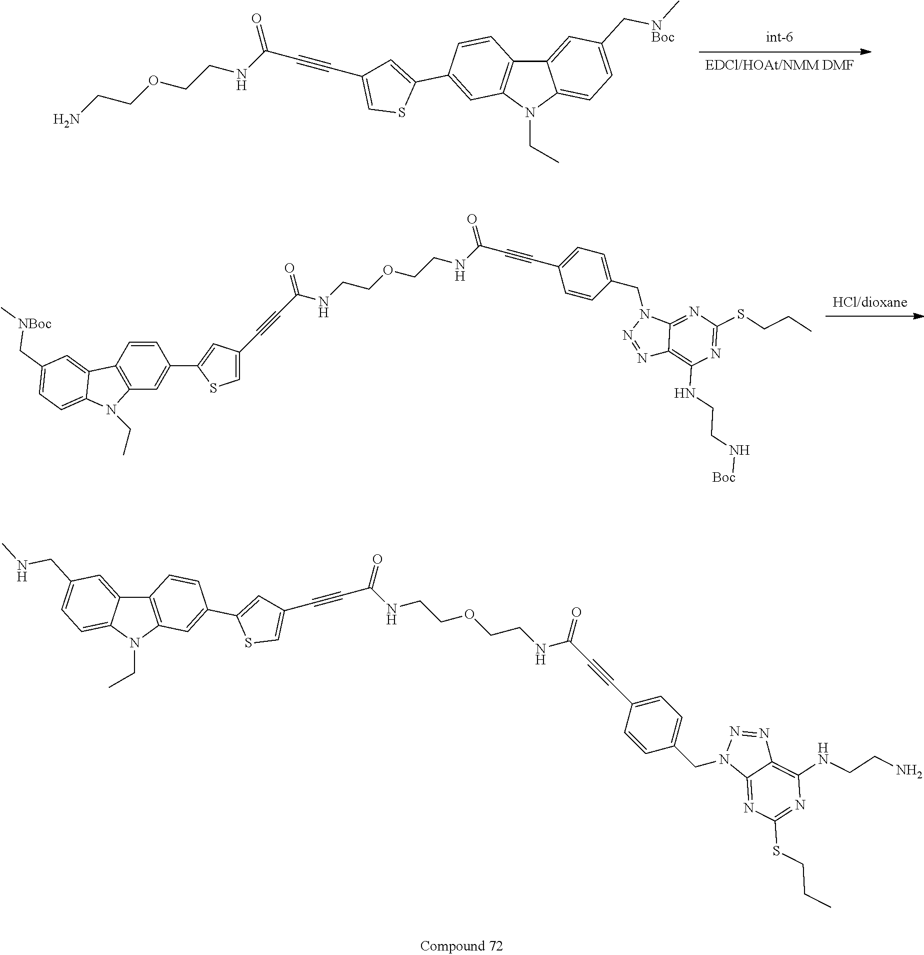

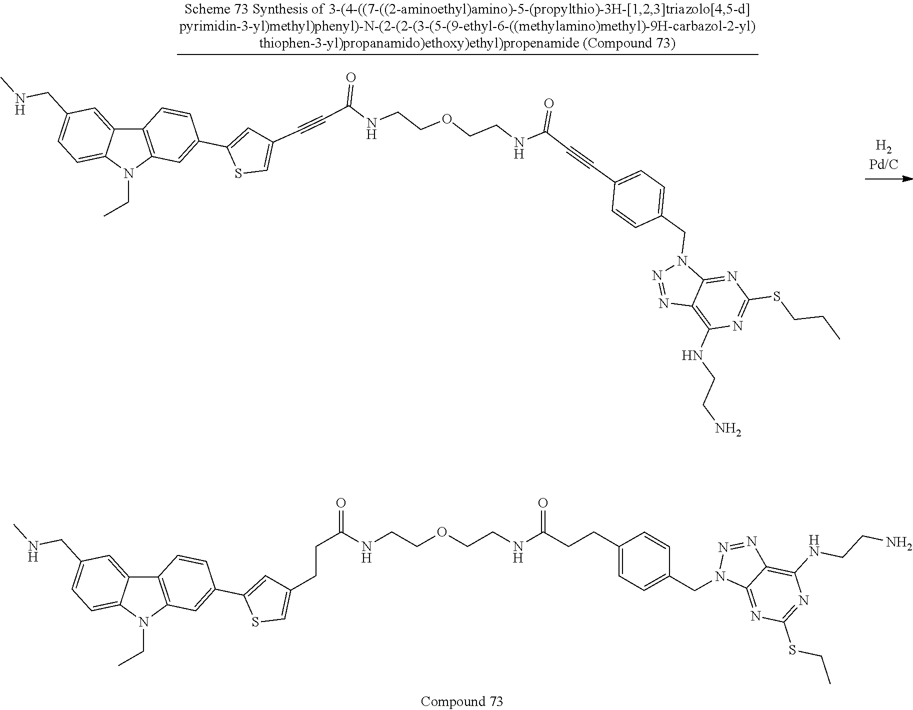

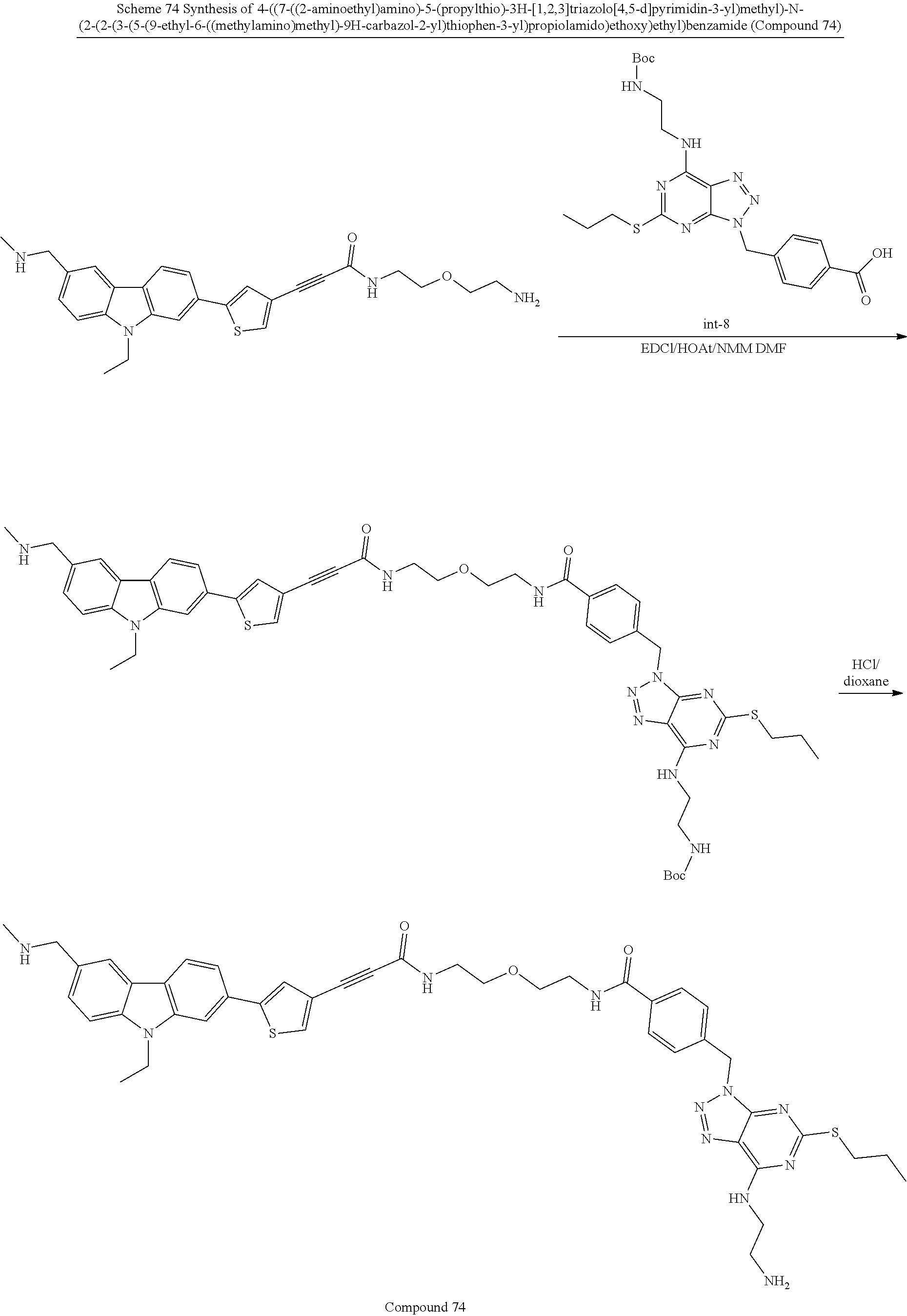

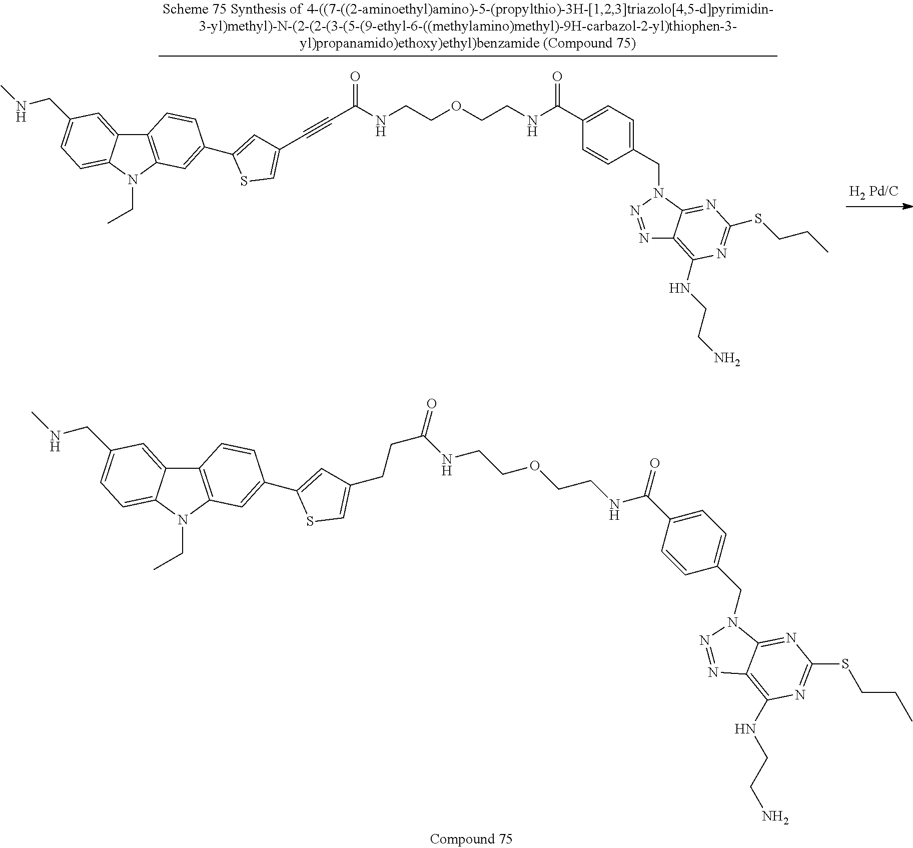

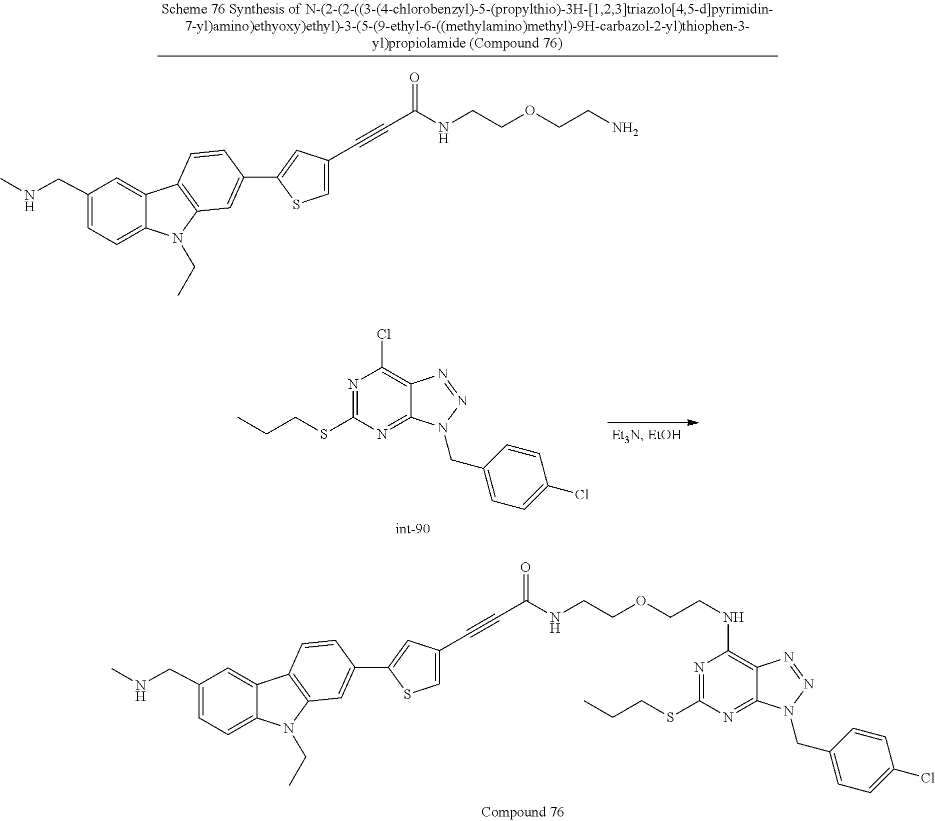 [SMILES: CCn1c2ccc(CNC)cc2c2ccc(-c3cc(C#CC(=O)NCCOCCN)cs3)cc21 ; CCCSc1nc(Cl)c2nnn(Cc3ccc(Cl)cc3)c2n1 ; CCCSc1nc(NCCOCCNC(=O)C#Cc2csc(-c3ccc4c5cc(CNC)ccc5n(CC)c4c3)c2)c2nnn(Cc3ccc(Cl)cc3)c2n1]

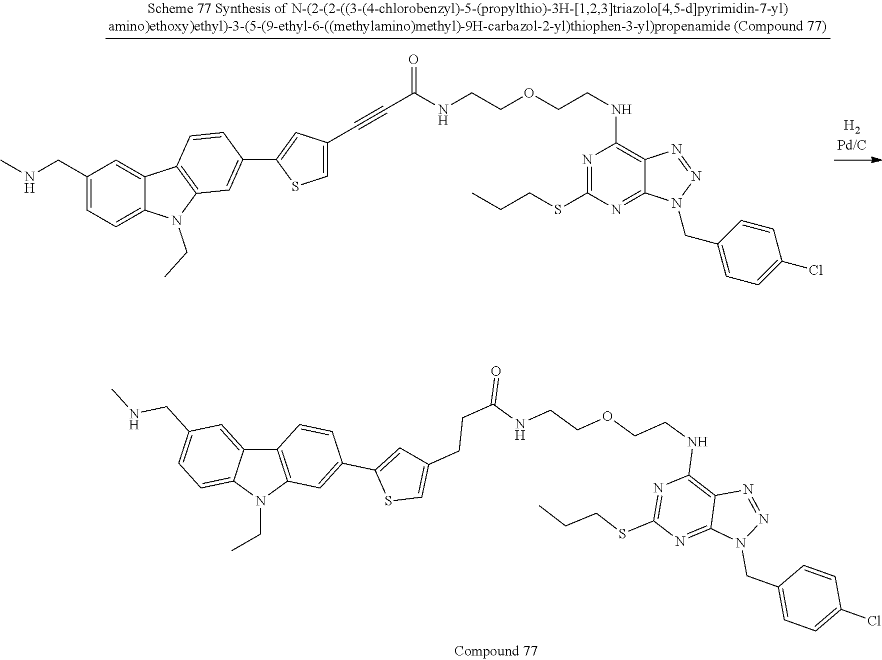 [SMILES: CCCSc1nc(NCCOCCNC(=O)C#Cc2csc(-c3ccc4c5cc(CNC)ccc5n(CC)c4c3)c2)c2nnn(Cc3ccc(Cl)cc3)c2n1 ; CCCSc1nc(NCCOCCNC(=O)CCc2csc(-c3ccc4c5cc(CNC)ccc5n(CC)c4c3)c2)c2nnn(Cc3ccc(Cl)cc3)c2n1]

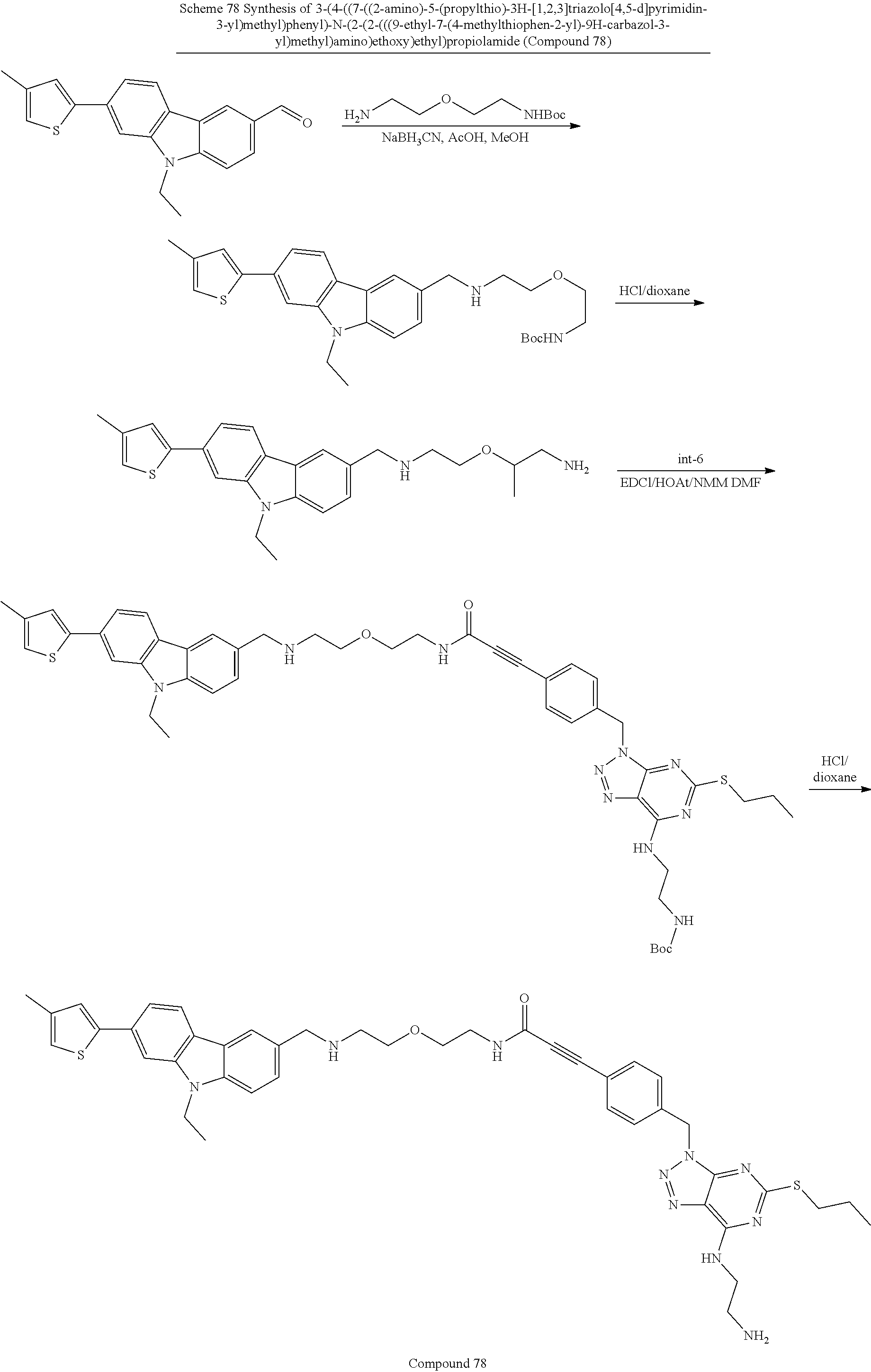

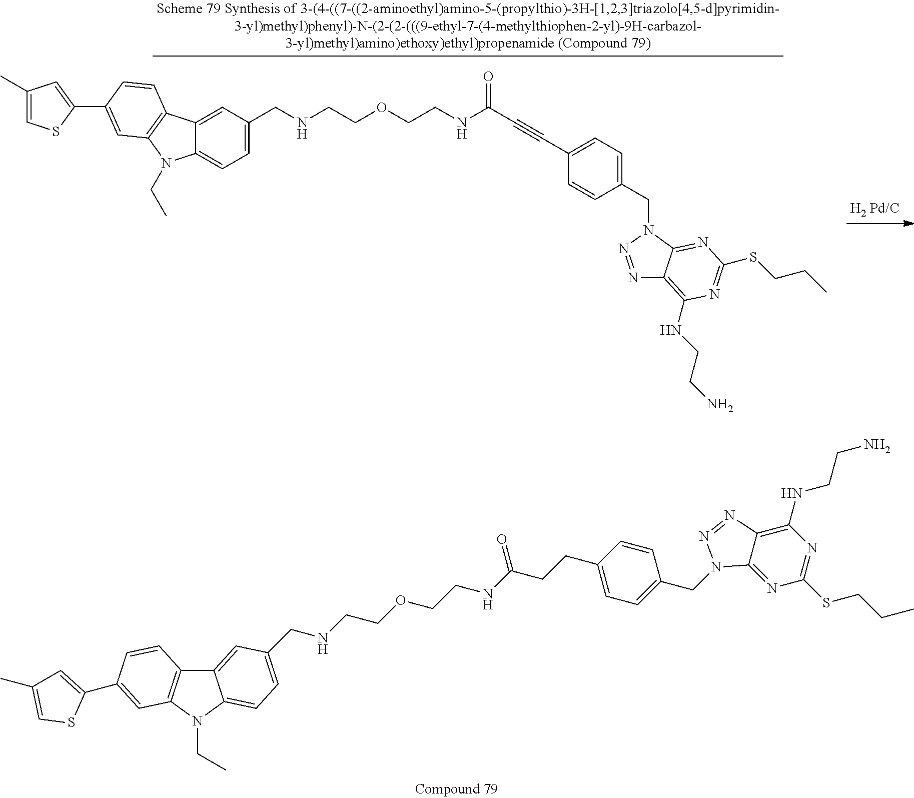

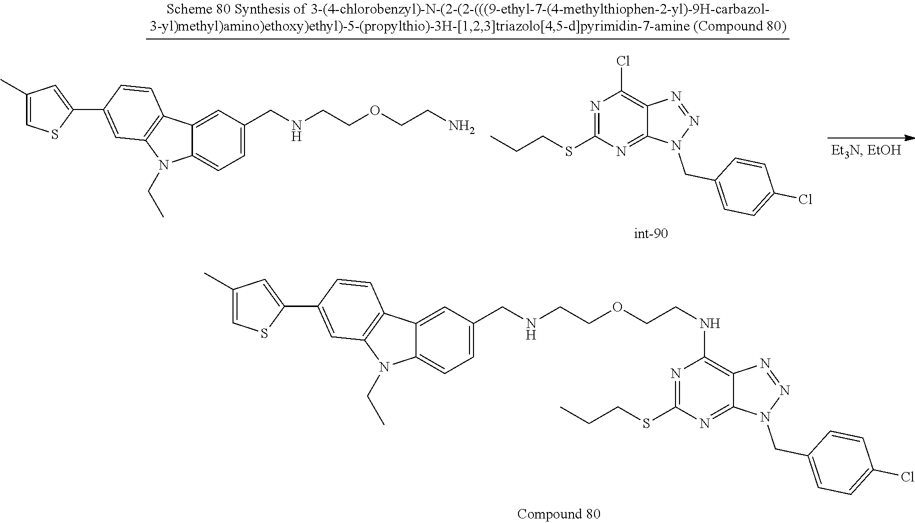

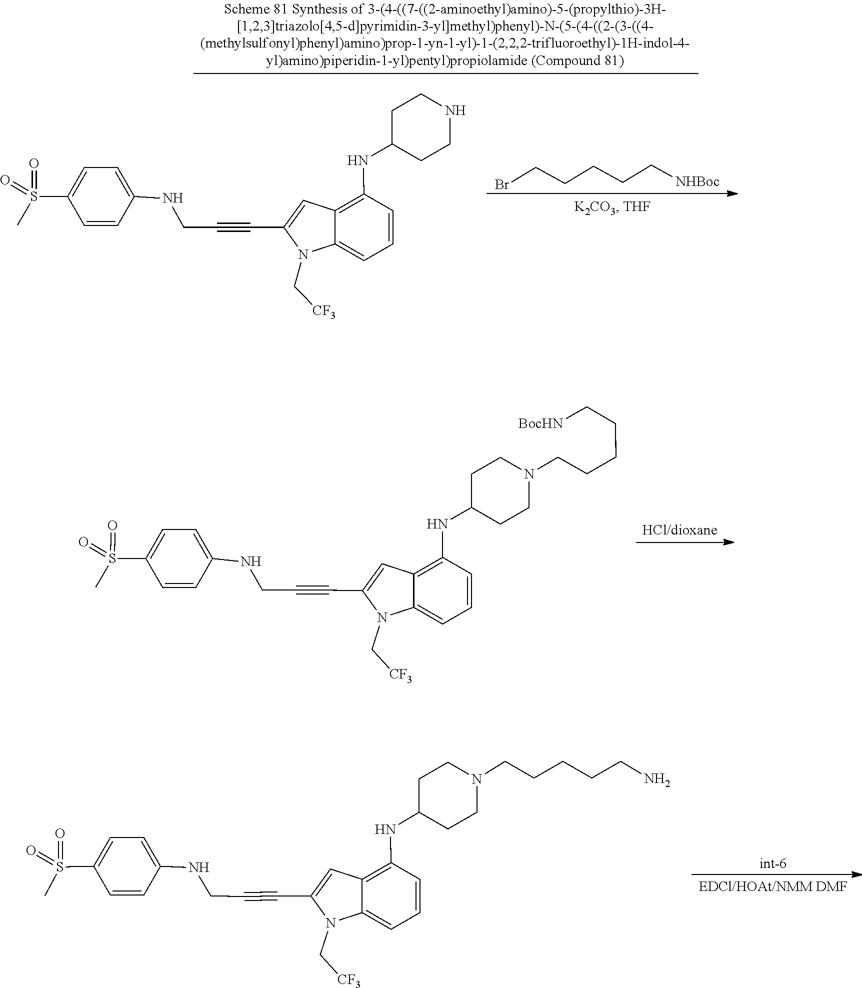 [SMILES: CS(=O)(=O)c1ccc(NCC#Cc2cc3c(NC4CCNCC4)cccc3n2CC(F)(F)F)cc1 ; CC(C)(C)OC(=O)NCCCCCBr ; CC(C)(C)OC(=O)NCCCCCN1CCC(Nc2cccc3c2cc(C#CCNc2ccc(S(C)(=O)=O)cc2)n3CC(F)(F)F)CC1 ; CS(=O)(=O)c1ccc(NCC#Cc2cc3c(NC4CCN(CCCCCN)CC4)cccc3n2CC(F)(F)F)cc1]

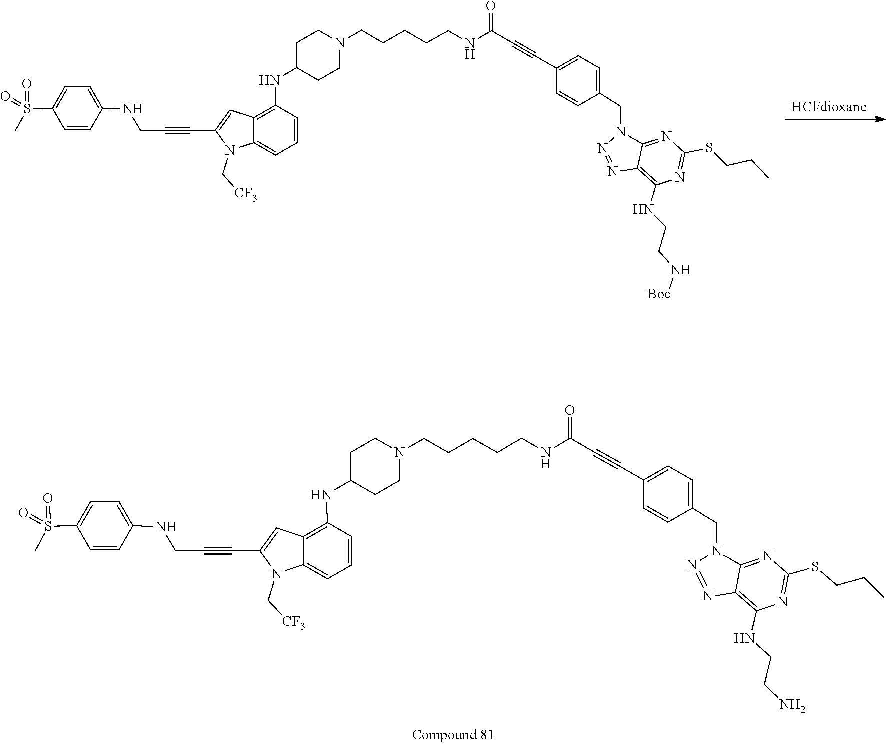

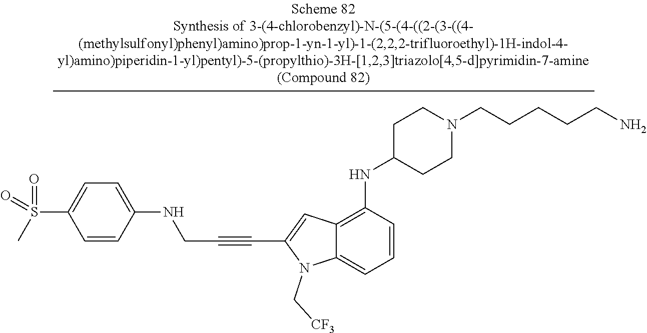

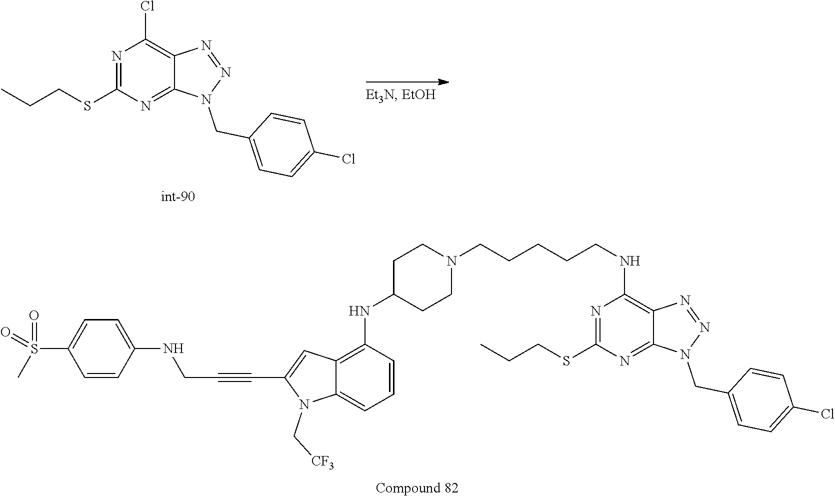 [SMILES: CCCSc1nc(Cl)c2nnn(Cc3ccc(Cl)cc3)c2n1 ; CCCSc1nc(NCCCCCN2CCC(Nc3cccc4c3cc(C#CCNc3ccc(S(C)(=O)=O)cc3)n4CC(F)(F)F)CC2)c2nnn(Cc3ccc(Cl)cc3)c2n1]

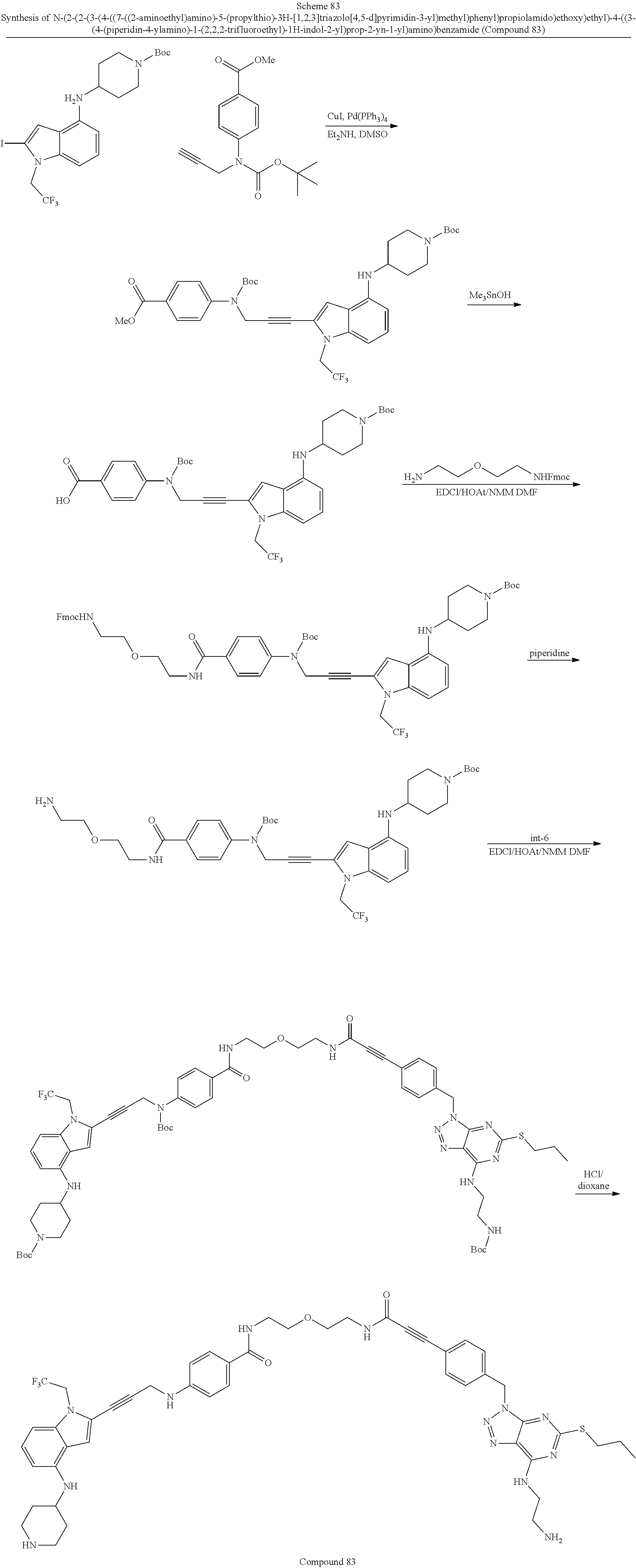

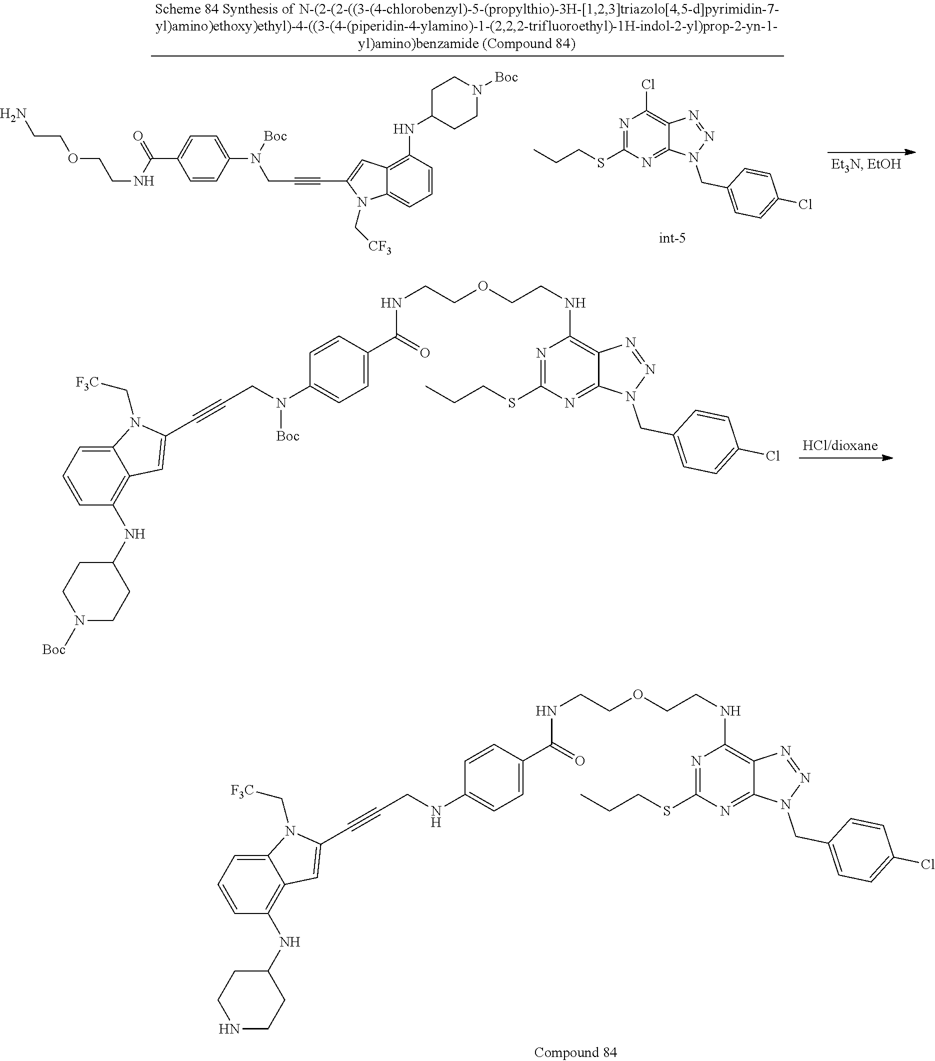 [SMILES: CC(C)(C)OC(=O)N1CCC(Nc2cccc3c2cc(C#CCN(C(=O)OC(C)(C)C)c2ccc(C(=O)NCCOCCN)cc2)n3CC(F)(F)F)CC1 ; CCCSc1nc(Cl)c2nnn(Cc3ccc(Cl)cc3)c2n1 ; CCCSc1nc(NCCOCCNC(=O)c2ccc(N(CC#Cc3cc4c(NC5CCN(C(=O)OC(C)(C)C)CC5)cccc4n3CC(F)(F)F)C(=O)OC(C)(C)C)cc2)c2nnn(Cc3ccc(Cl)cc3)c2n1 ; CCCSc1nc(NCCOCCNC(=O)c2ccc(NCC#Cc3cc4c(NC5CCNCC5)cccc4n3CC(F)(F)F)cc2)c2nnn(Cc3ccc(Cl)cc3)c2n1]

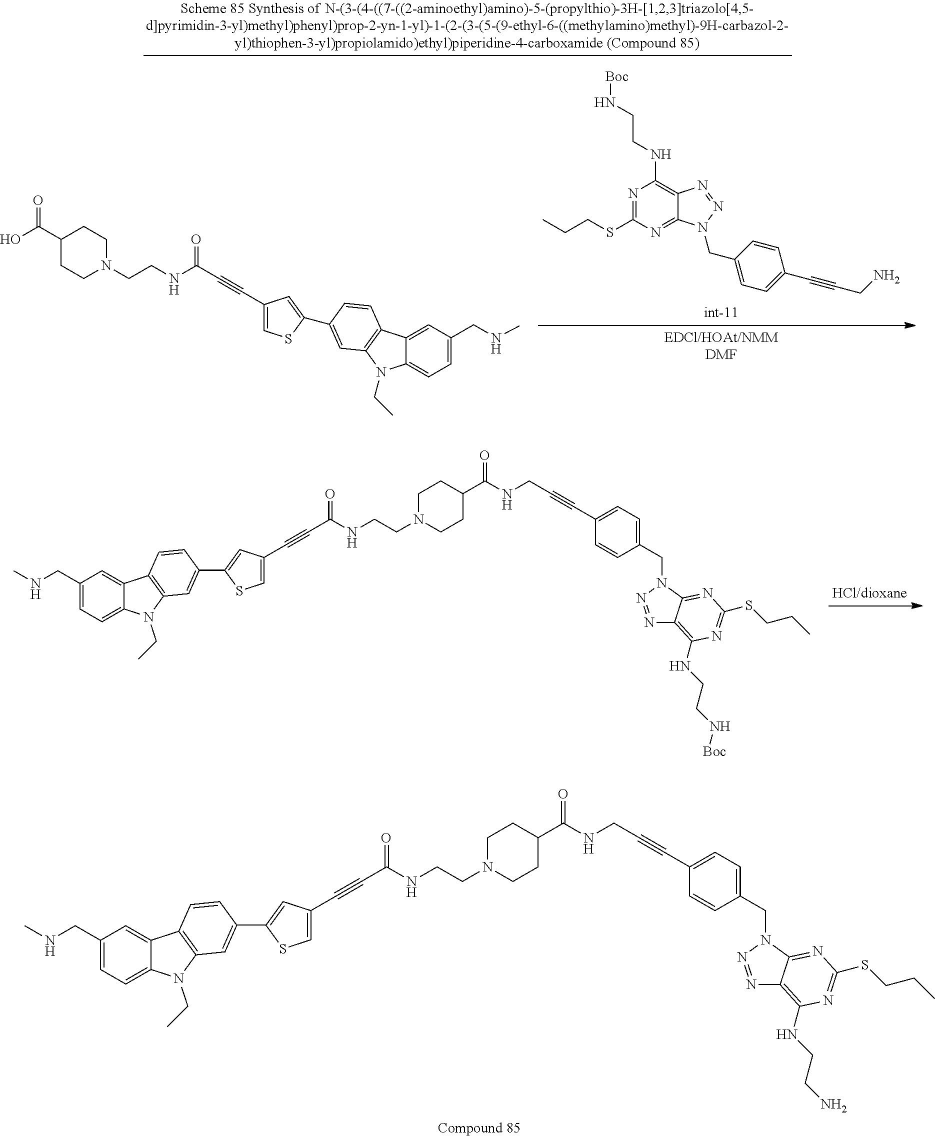

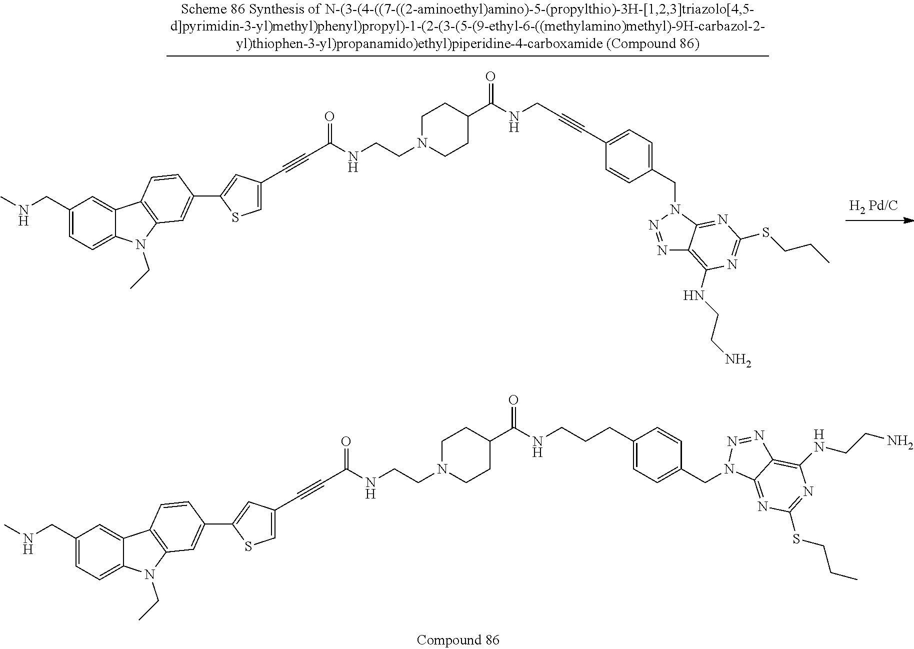

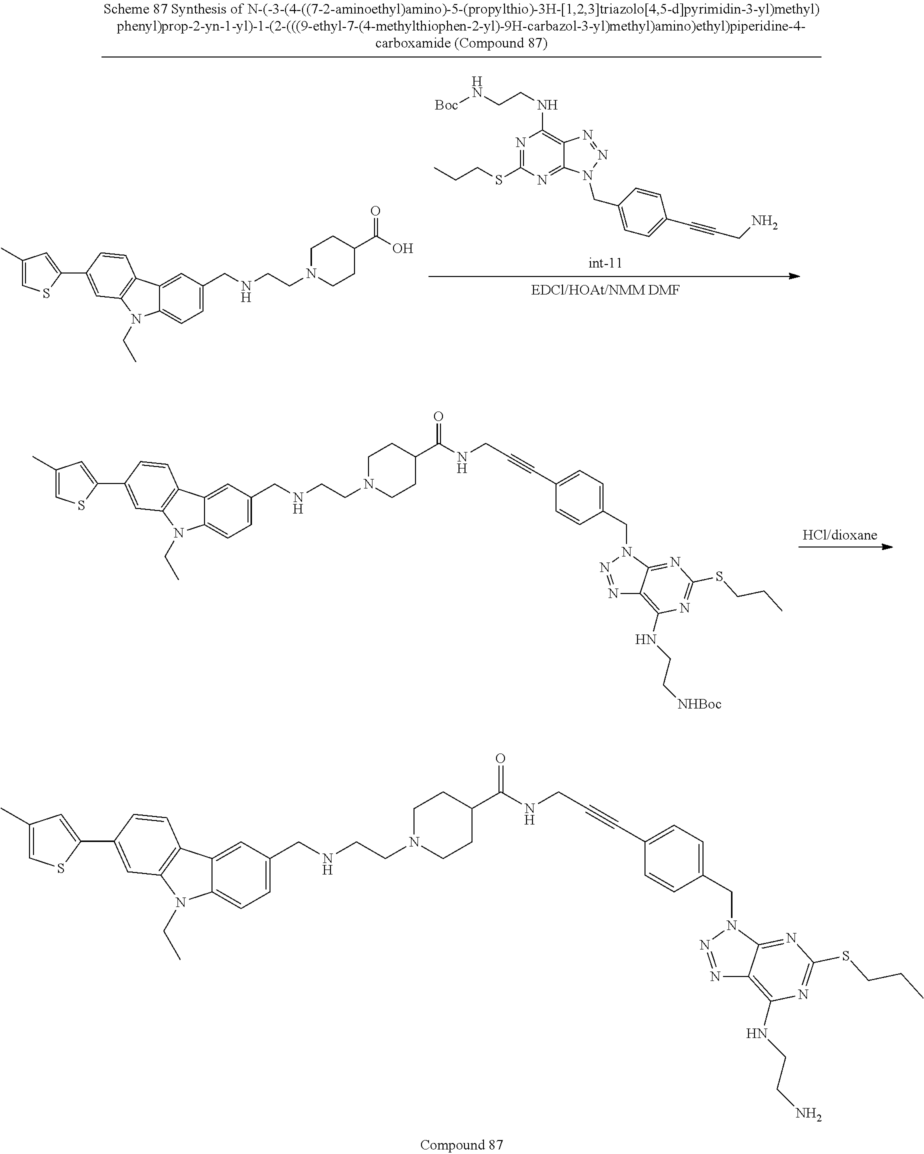

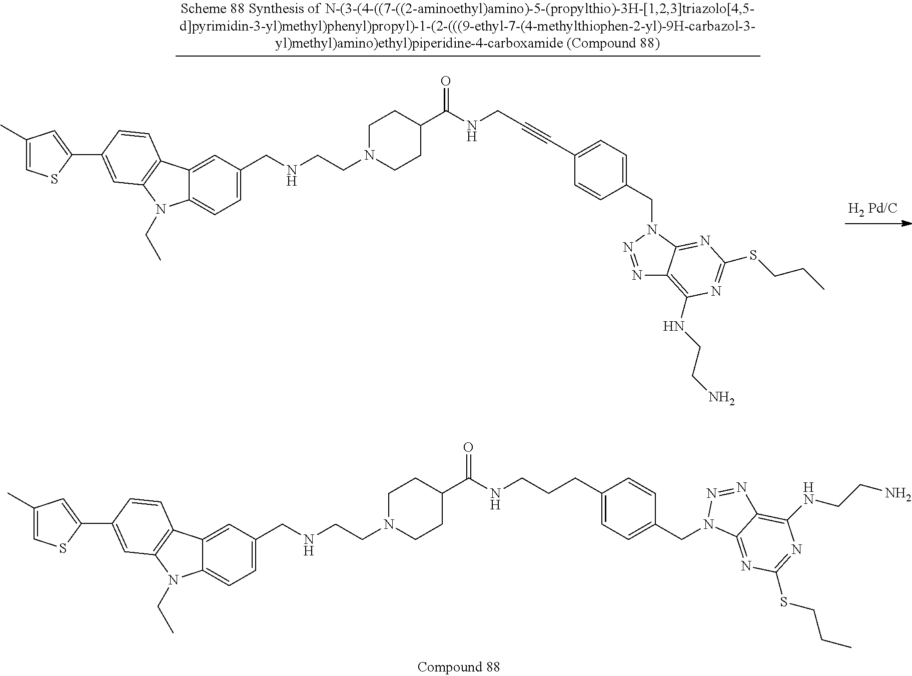

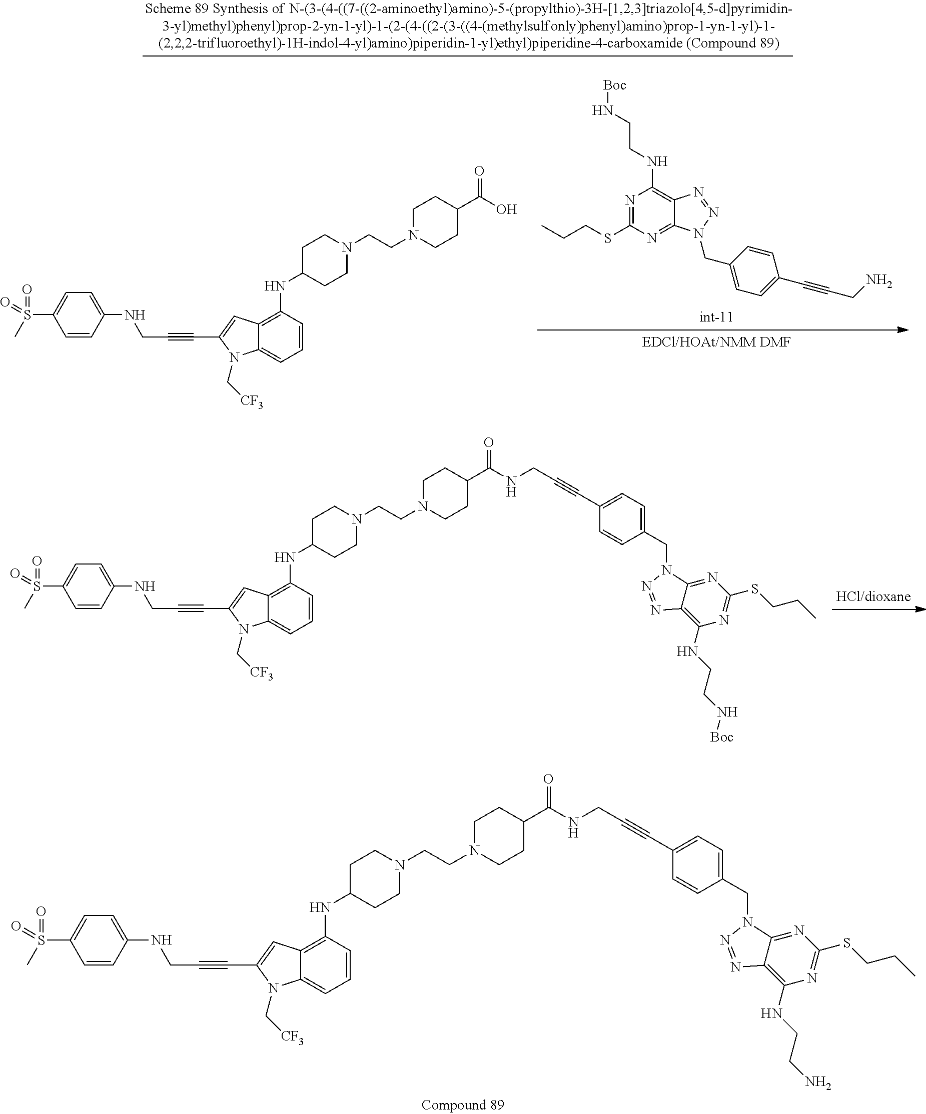 [SMILES: CS(=O)(=O)c1ccc(NCC#Cc2cc3c(NC4CCN(CCN5CCC(C(=O)O)CC5)CC4)cccc3n2CC(F)(F)F)cc1 ; CCCSc1nc(NCCNC(=O)OC(C)(C)C)c2nnn(Cc3ccc(C#CCN)cc3)c2n1 ; CCCSc1nc(NCCNC(=O)OC(C)(C)C)c2nnn(Cc3ccc(C#CCNC(=O)C4CCN(CCN5CCC(Nc6cccc7c6cc(C#CCNc6ccc(S(C)(=O)=O)cc6)n7CC(F)(F)F)CC5)CC4)cc3)c2n1 ; CCCSc1nc(NCCN)c2nnn(Cc3ccc(C#CCNC(=O)C4CCN(CCN5CCC(Nc6cccc7c6cc(C#CCNc6ccc(S(C)(=O)=O)cc6)n7CC(F)(F)F)CC5)CC4)cc3)c2n1]

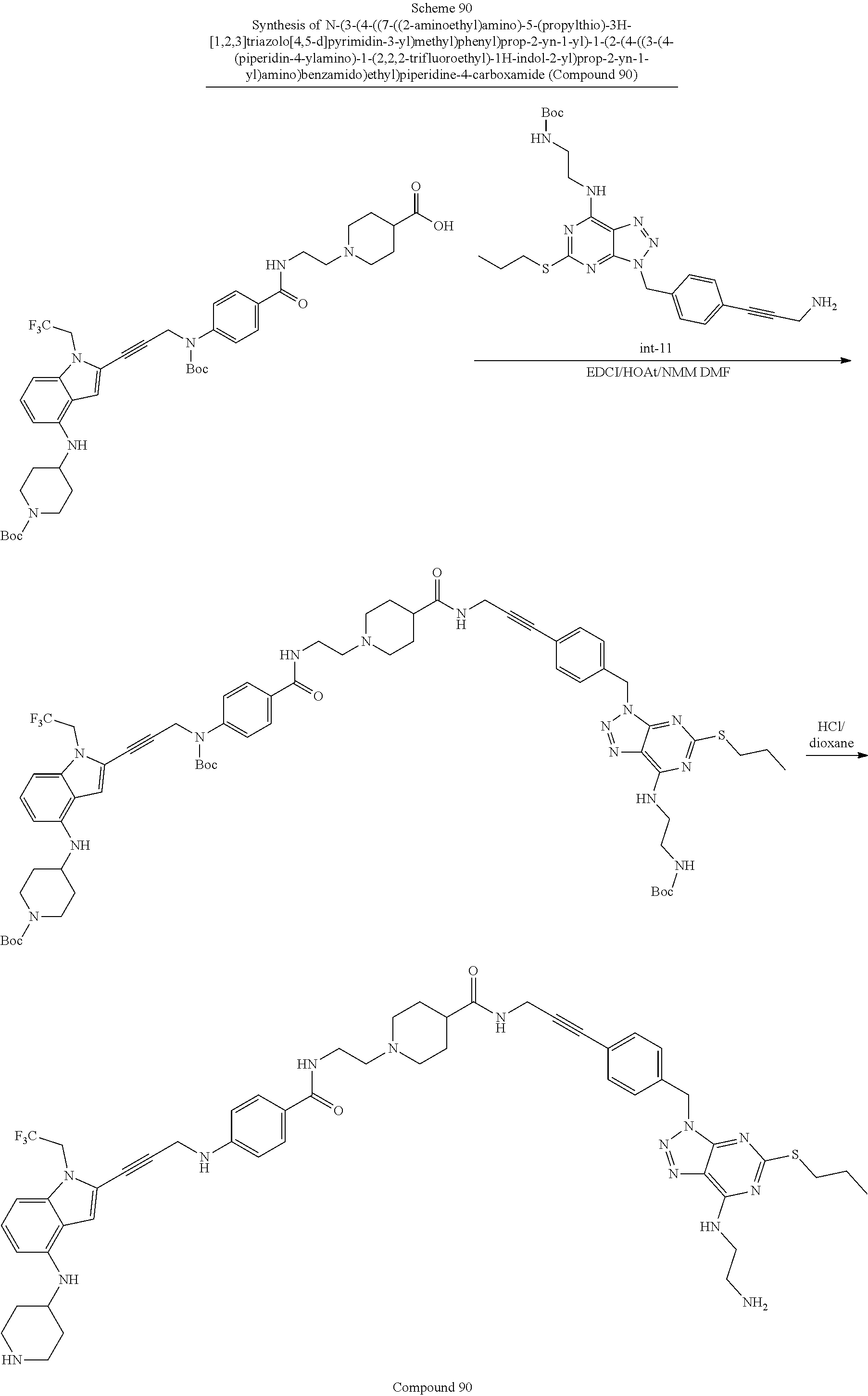 [SMILES: CC(C)(C)OC(=O)N1CCC(Nc2cccc3c2cc(C#CCN(C(=O)OC(C)(C)C)c2ccc(C(=O)NCCN4CCC(C(=O)O)CC4)cc2)n3CC(F)(F)F)CC1 ; CCCSc1nc(NCCNC(=O)OC(C)(C)C)c2nnn(Cc3ccc(C#CCN)cc3)c2n1 ; CCCSc1nc(NCCNC(=O)OC(C)(C)C)c2nnn(Cc3ccc(C#CCNC(=O)C4CCN(CCNC(=O)c5ccc(N(CC#Cc6cc7c(NC8CCN(C(=O)OC(C)(C)C)CC8)cccc7n6CC(F)(F)F)C(=O)OC(C)(C)C)cc5)CC4)cc3)c2n1 ; CCCSc1nc(NCCN)c2nnn(Cc3ccc(C#CCNC(=O)C4CCN(CCNC(=O)c5ccc(NCC#Cc6cc7c(NC8CCNCC8)cccc7n6CC(F)(F)F)cc5)CC4)cc3)c2n1]

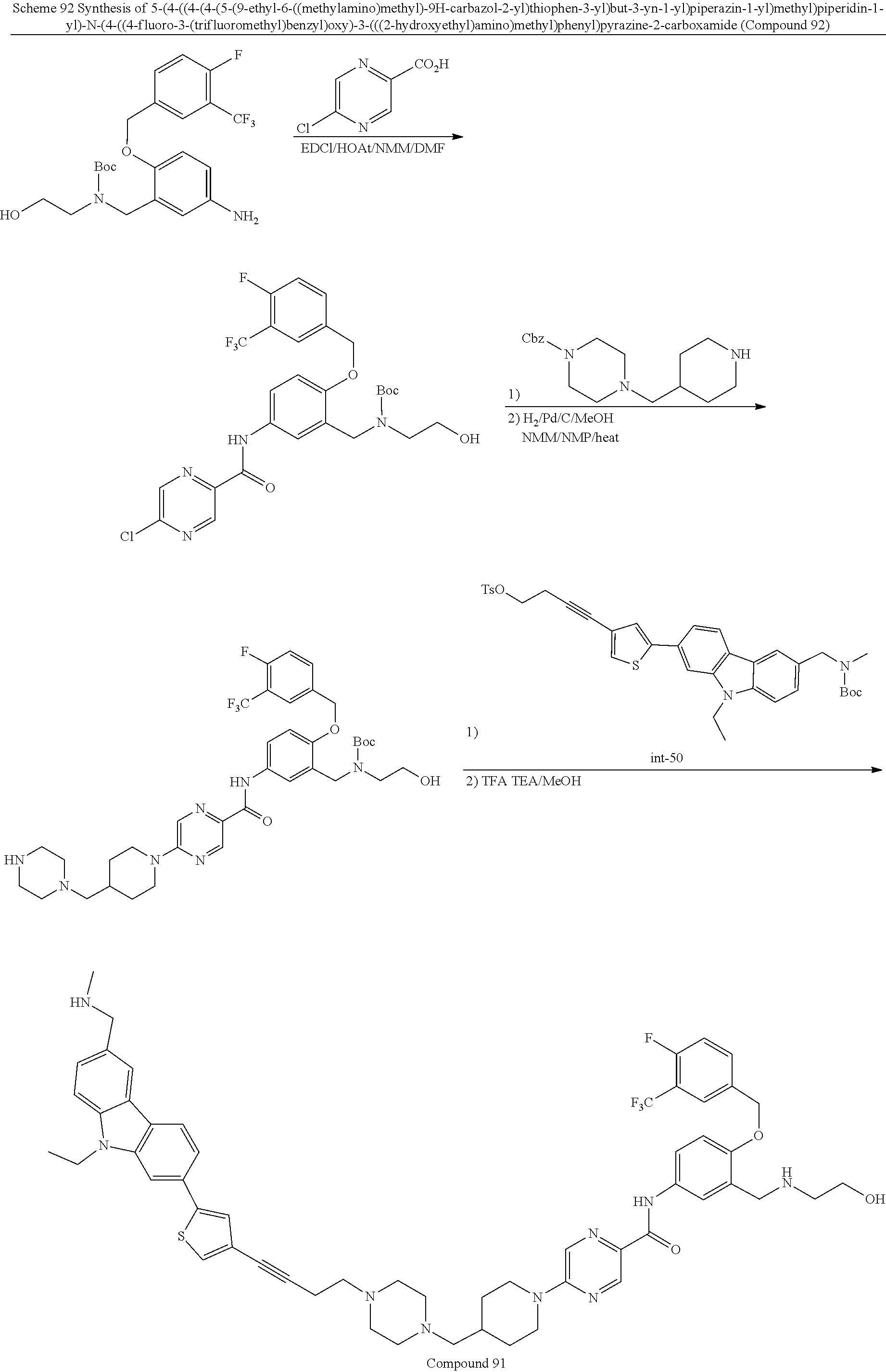

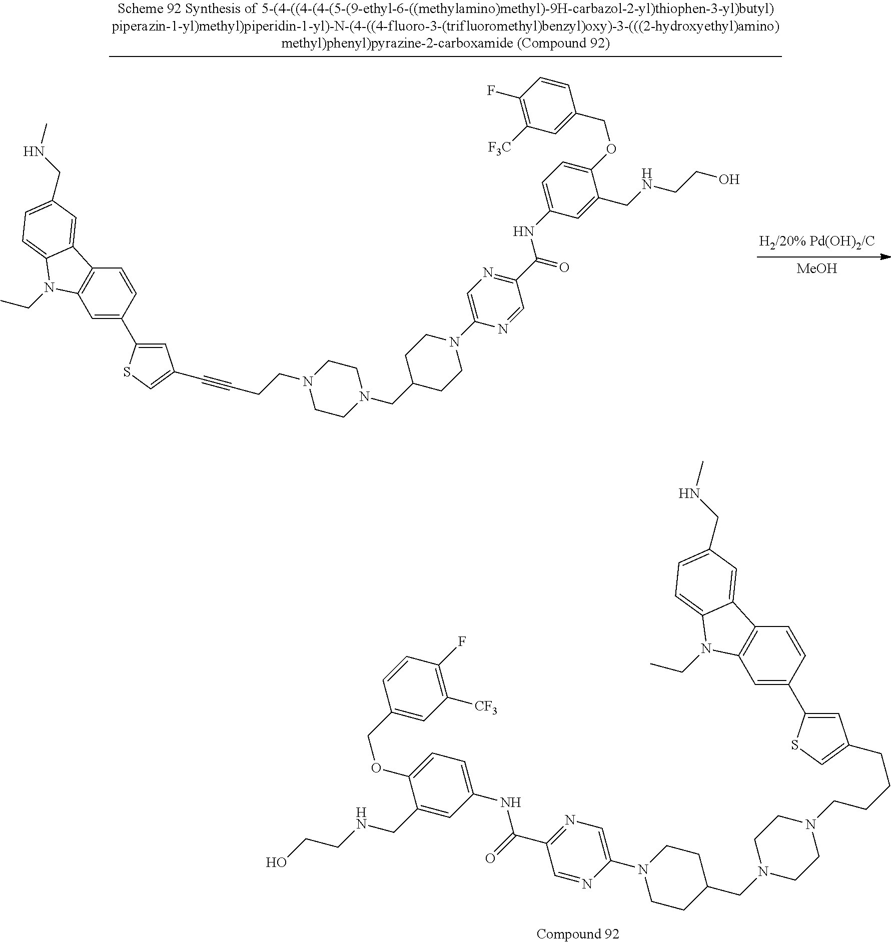 [SMILES: "MeOH" ; CCn1c2ccc(CNC)cc2c2ccc(-c3cc(C#CCCN4CCN(CC5CCN(c6cnc(C(=O)Nc7ccc(OCc8ccc(F)c(C(F)(F)F)c8)c(CNCCO)c7)cn6)CC5)CC4)cs3)cc21 ; CCn1c2ccc(CNC)cc2c2ccc(-c3cc(CCCCN4CCN(CC5CCN(c6cnc(C(=O)Nc7ccc(OCc8ccc(F)c(C(F)(F)F)c8)c(CNCCO)c7)cn6)CC5)CC4)cs3)cc21]

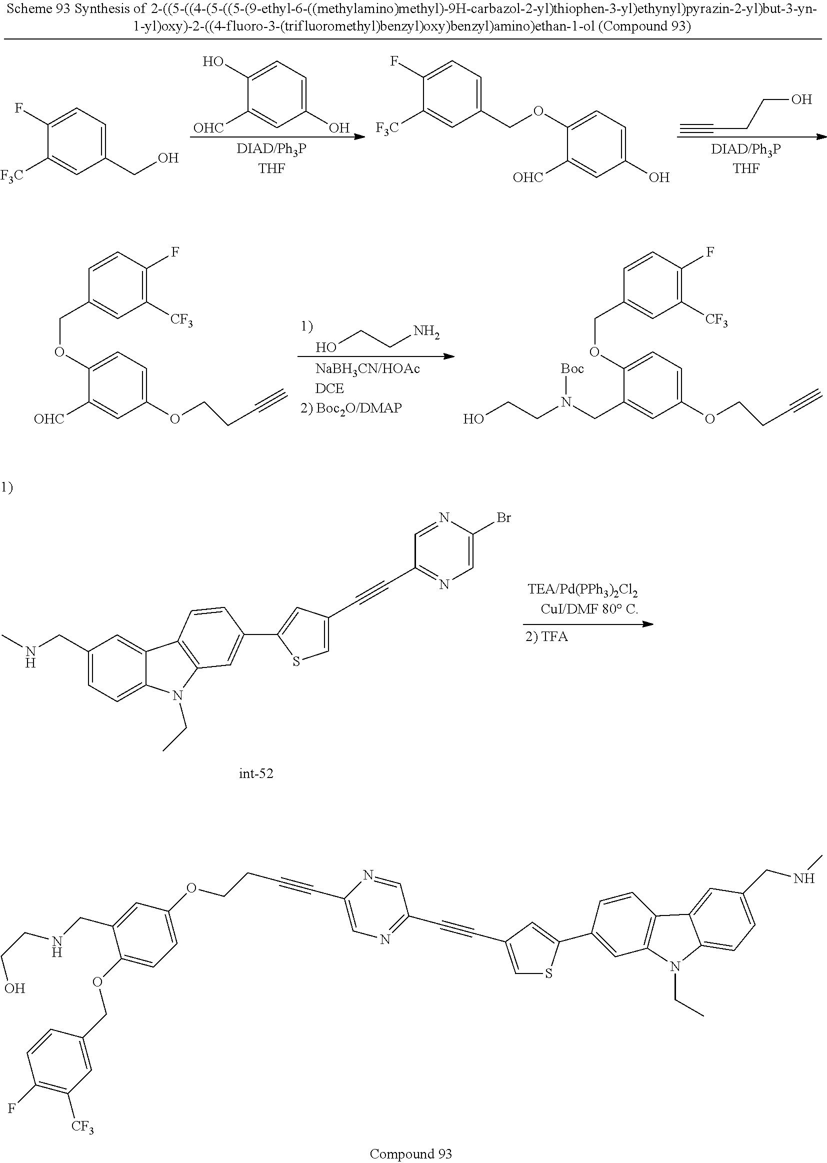

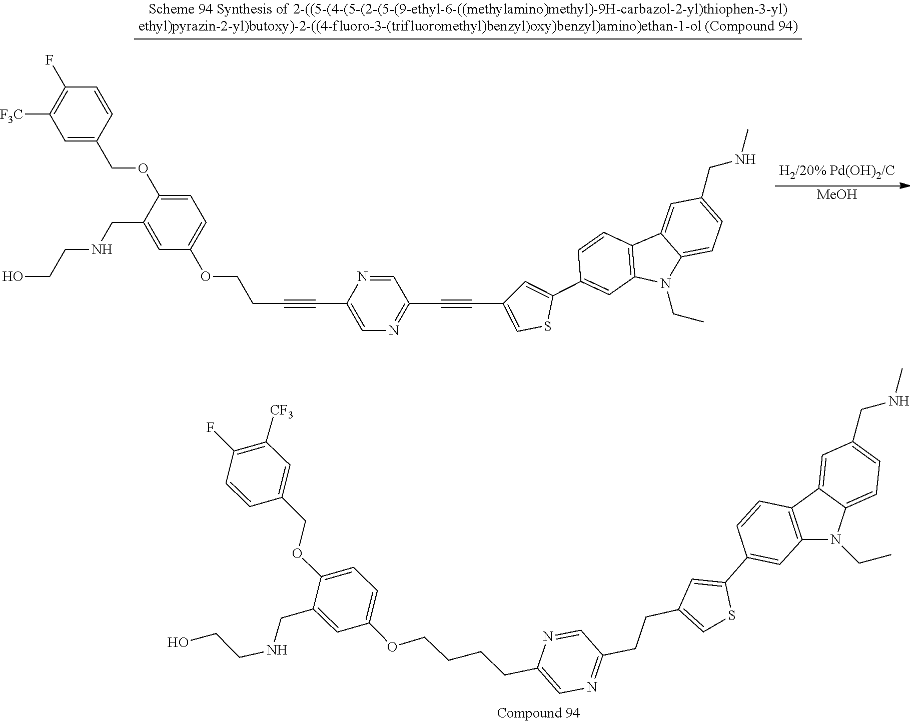 [SMILES: "MeOH" ; CCn1c2ccc(CNC)cc2c2ccc(-c3cc(C#Cc4cnc(C#CCCOc5ccc(OCc6ccc(F)c(C(F)(F)F)c6)c(CNCCO)c5)cn4)cs3)cc21 ; CCn1c2ccc(CNC)cc2c2ccc(-c3cc(CCc4cnc(CCCCOc5ccc(OCc6ccc(F)c(C(F)(F)F)c6)c(CNCCO)c5)cn4)cs3)cc21]

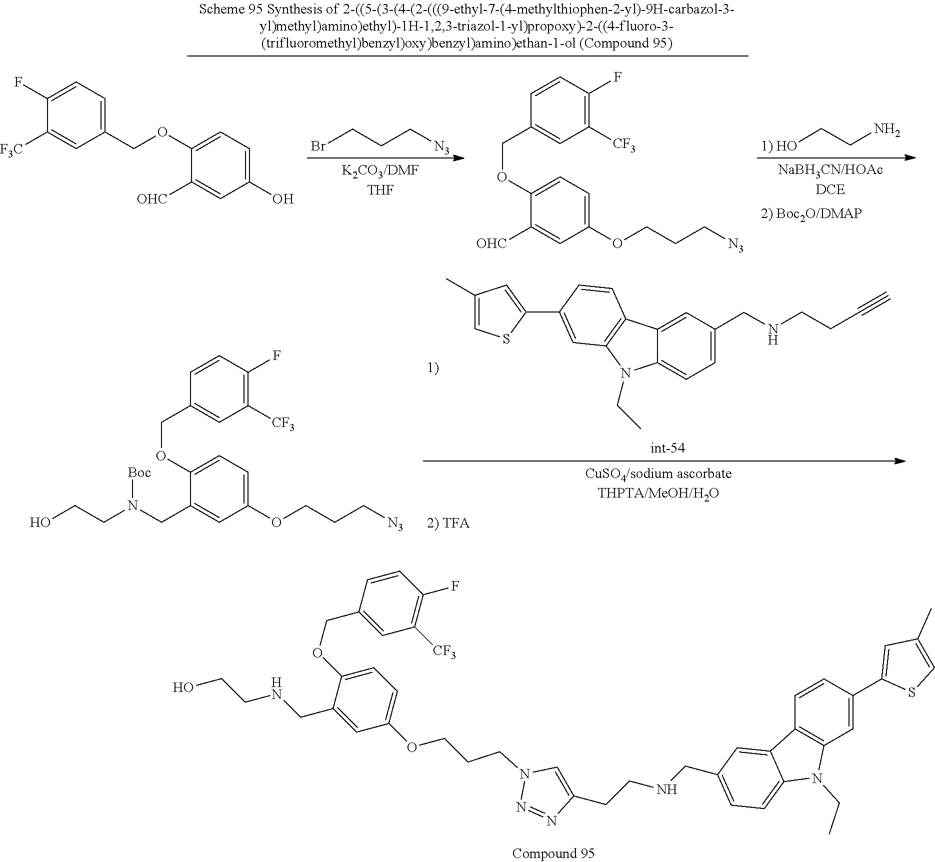

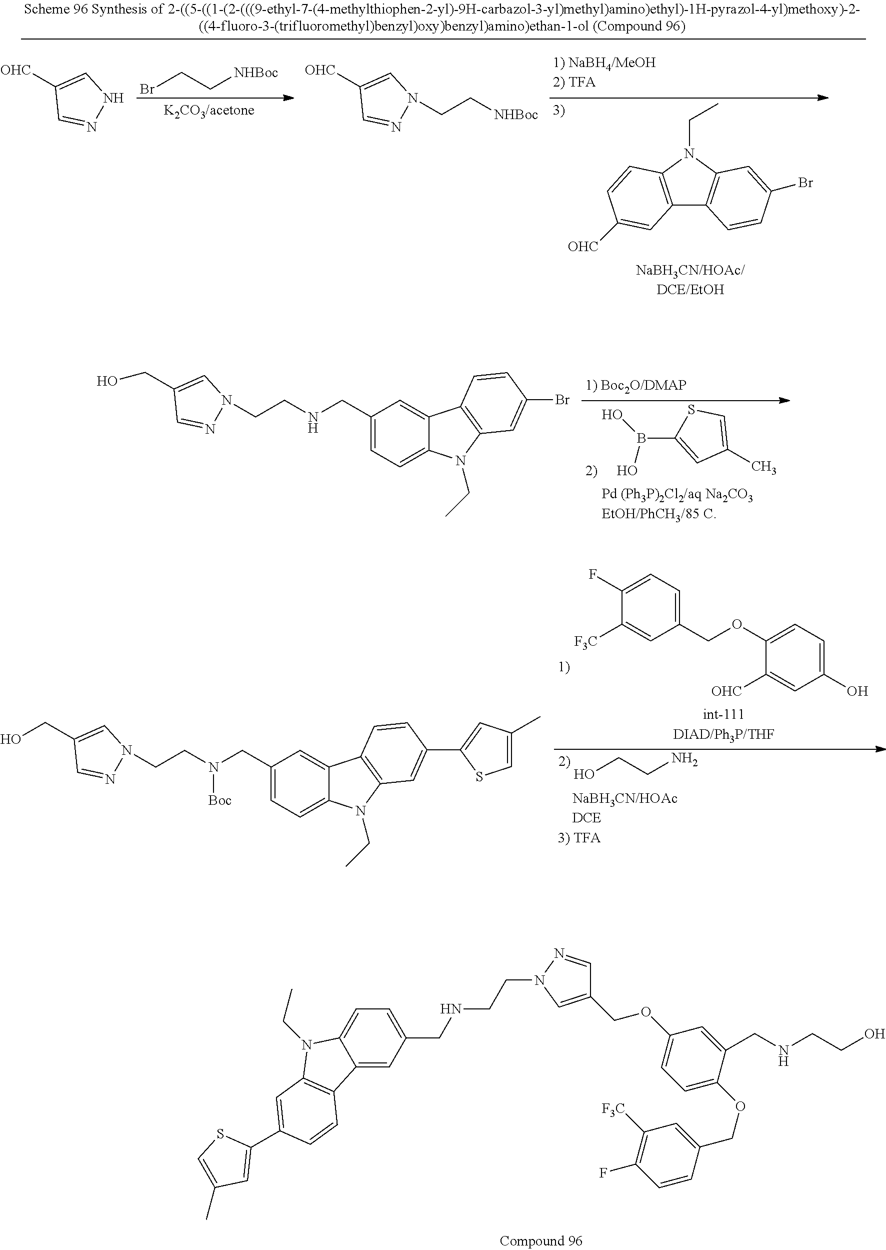

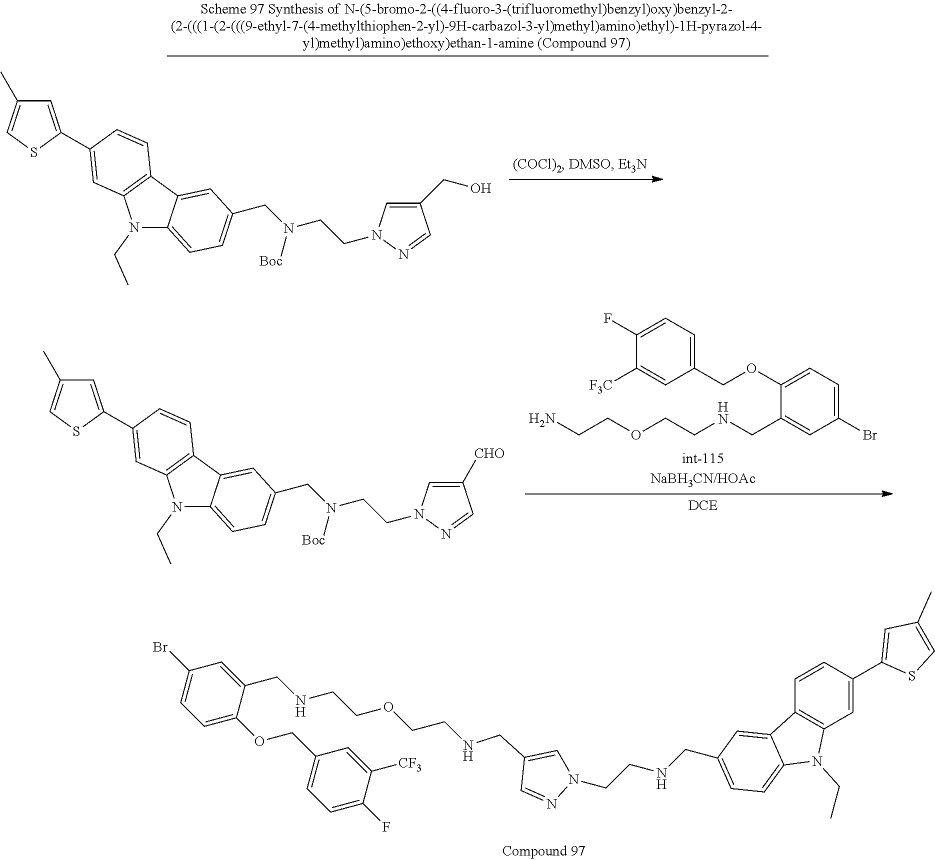 [SMILES: CCn1c2ccc(CN(CCn3cc(CO)cn3)C(=O)OC(C)(C)C)cc2c2ccc(-c3cc(C)cs3)cc21 ; CCn1c2ccc(CN(CCn3cc(C=O)cn3)C(=O)OC(C)(C)C)cc2c2ccc(-c3cc(C)cs3)cc21 ; NCCOCCNCc1cc(Br)ccc1OCc1ccc(F)c(C(F)(F)F)c1 ; CCn1c2ccc(CNCCn3cc(CNCCOCCNCc4cc(Br)ccc4OCc4ccc(F)c(C(F)(F)F)c4)cn3)cc2c2ccc(-c3cc(C)cs3)cc21]

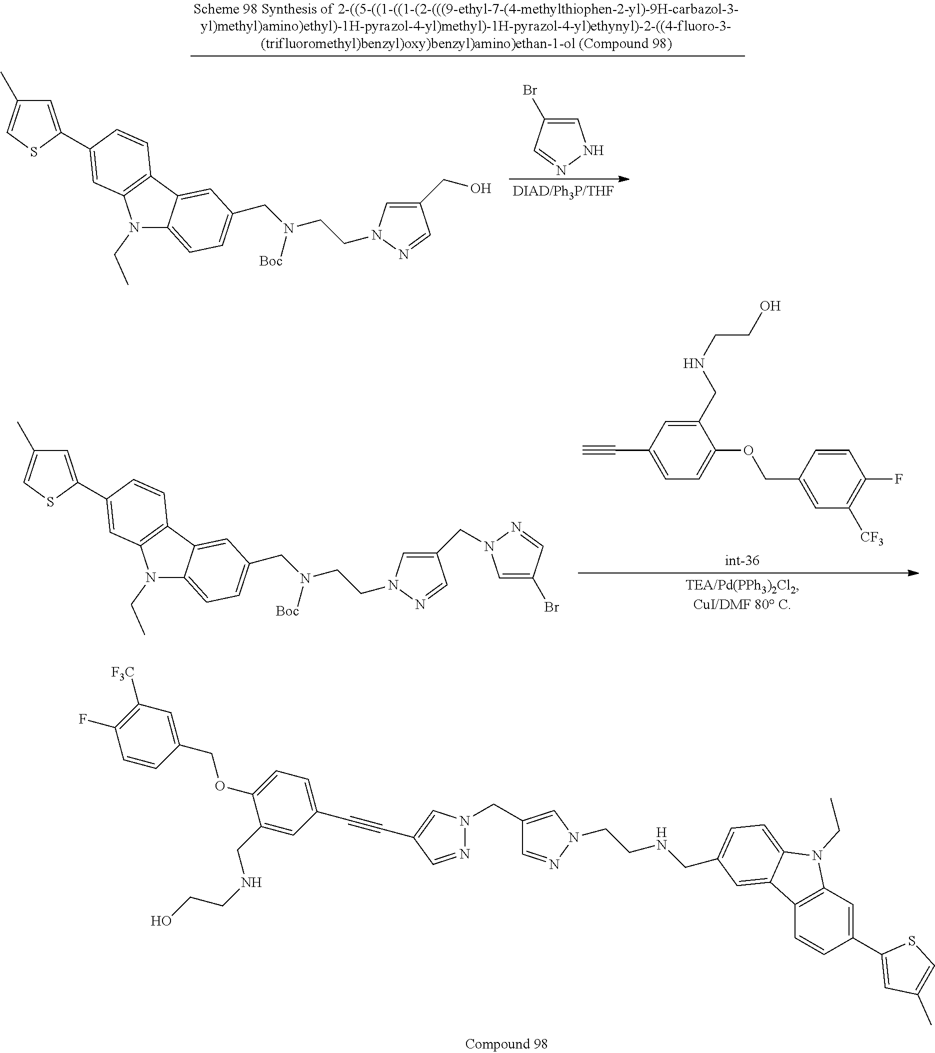

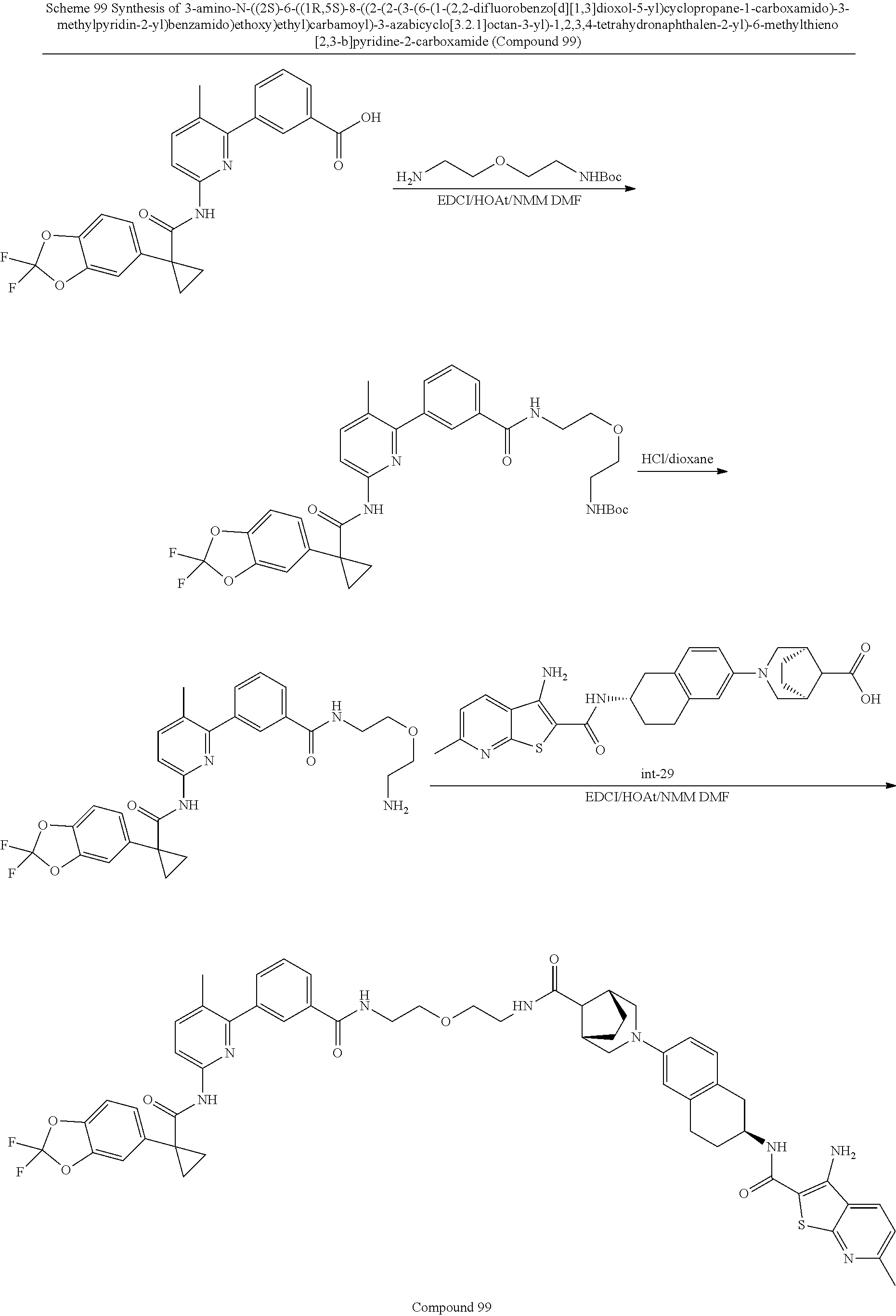

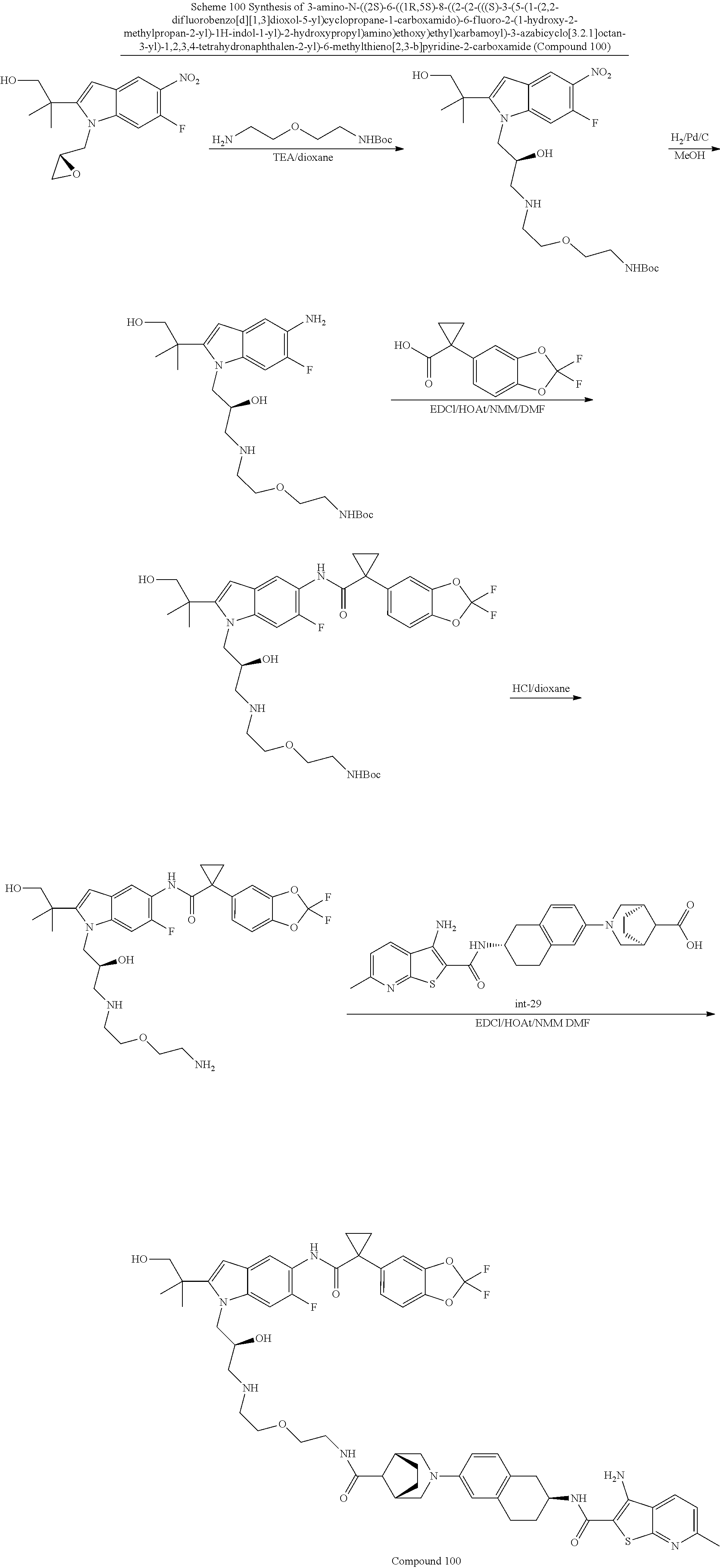

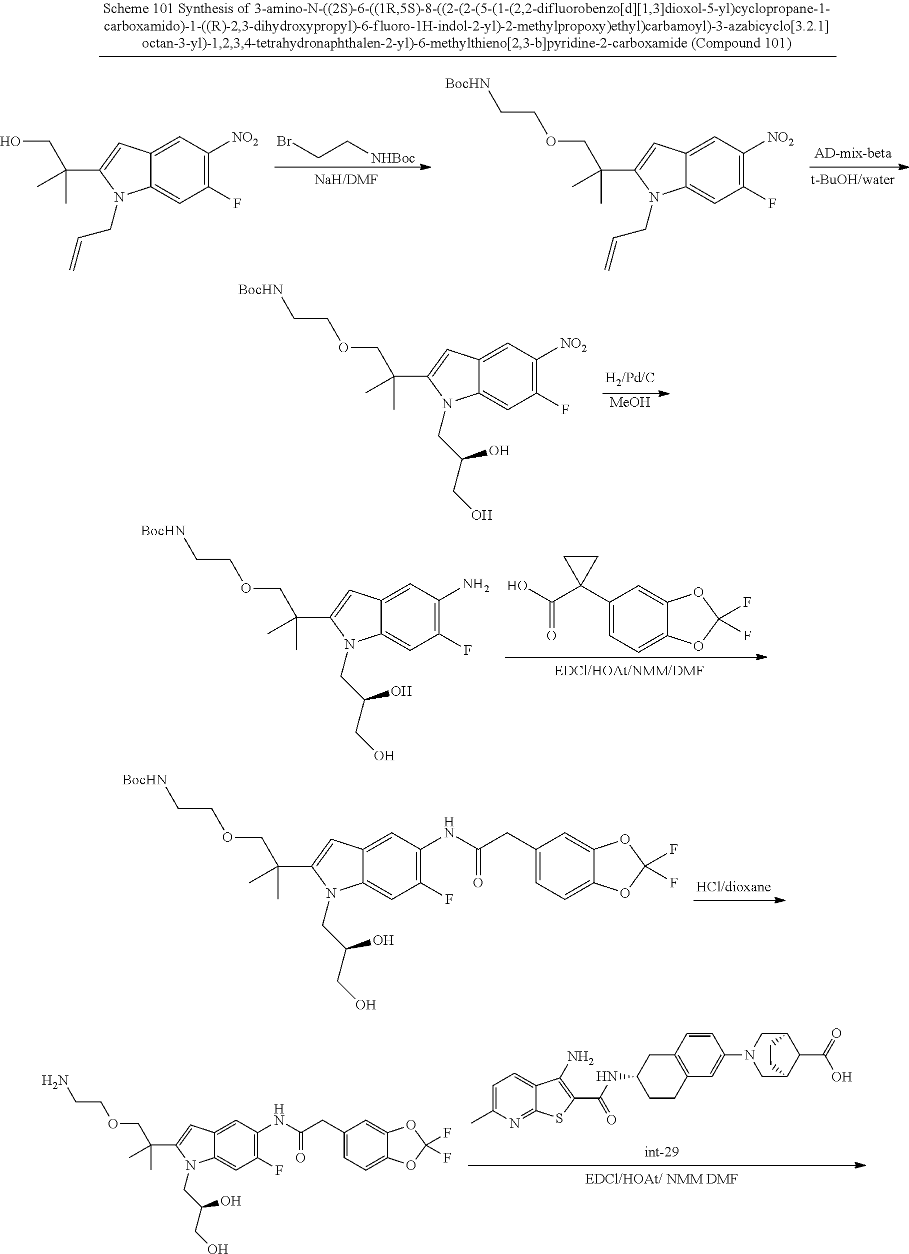

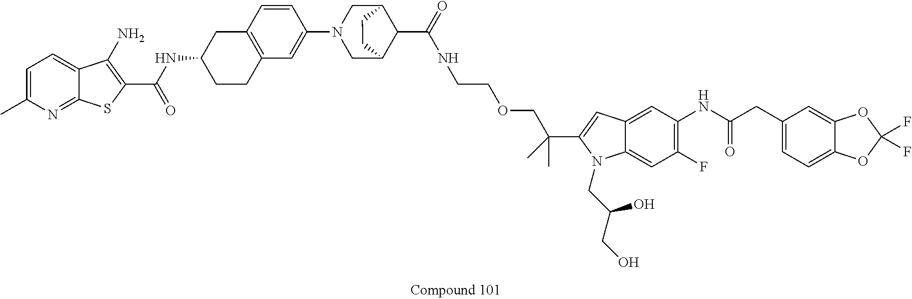 [SMILES: Cc1ccc2c(N)c(C(=O)N[C@H]3CCc4cc(N5C[C@H]6CC[C@@H](C5)C6C(=O)NCCOCC(C)(C)c5cc6cc(NC(=O)Cc7ccc8c(c7)OC(F)(F)O8)c(F)cc6n5C[C@@H](O)CO)ccc4C3)sc2n1]

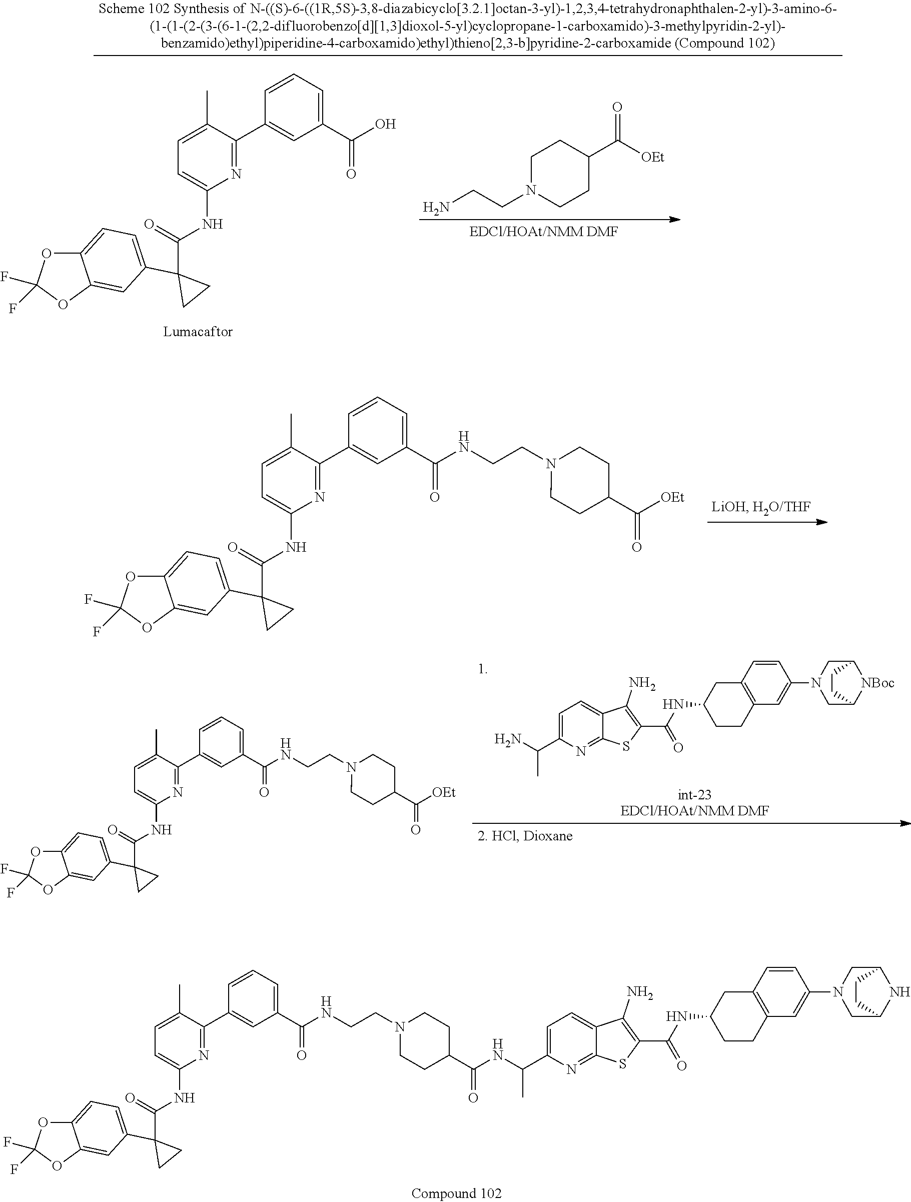 [SMILES: Cc1ccc(NC(=O)C2(c3ccc4c(c3)OC(F)(F)O4)CC2)nc1-c1cccc(C(=O)O)c1 ; CCOC(=O)C1CCN(CCN)CC1 ; CCOC(=O)C1CCN(CCNC(=O)c2cccc(-c3nc(NC(=O)C4(c5ccc6c(c5)OC(F)(F)O6)CC4)ccc3C)c2)CC1 ; CCOC(=O)C1CCN(CCNC(=O)c2cccc(-c3nc(NC(=O)C4(c5ccc6c(c5)OC(F)(F)O6)CC4)ccc3C)c2)CC1 ; CC(N)c1ccc2c(N)c(C(=O)N[C@H]3CCc4cc(N5C[C@H]6CC[C@@H](C5)N6C(=O)OC(C)(C)C)ccc4C3)sc2n1 ; Cc1ccc(NC(=O)C2(c3ccc4c(c3)OC(F)(F)O4)CC2)nc1-c1cccc(C(=O)NCCN2CCC(C(=O)NC(C)c3ccc4c(N)c(C(=O)N[C@H]5CCc6cc(N7C[C@H]8CC[C@@H](C7)N8)ccc6C5)sc4n3)CC2)c1]

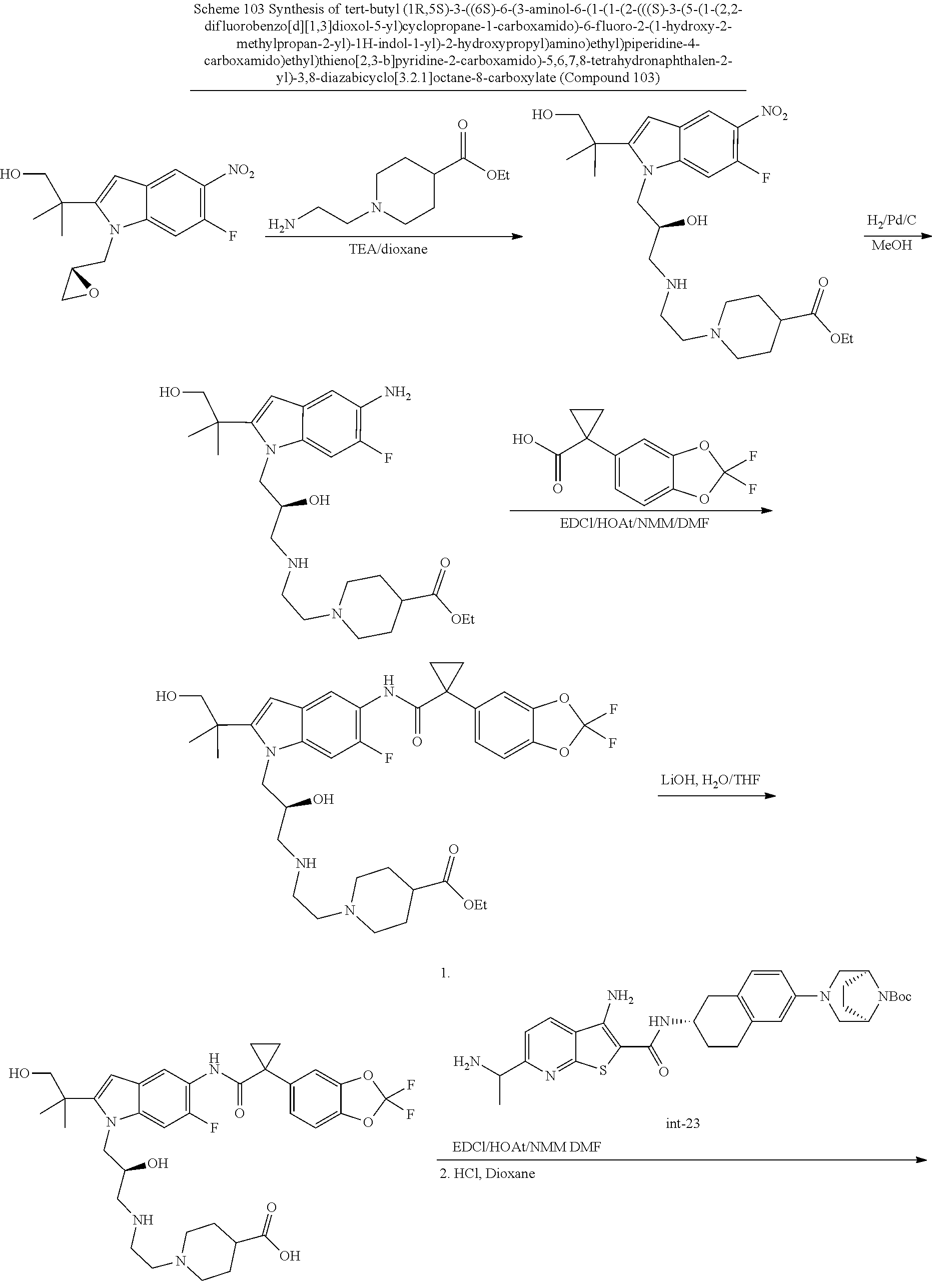

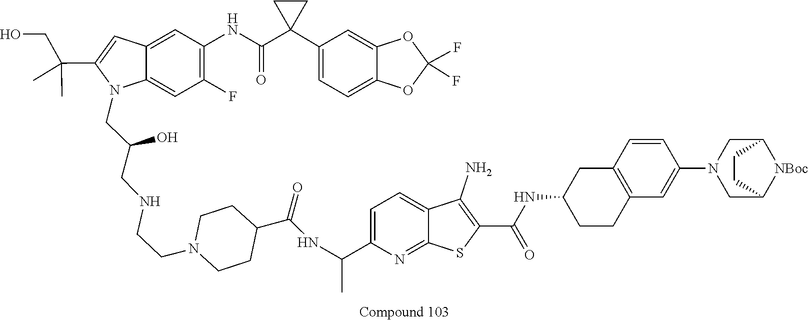 [SMILES: CC(NC(=O)C1CCN(CCNC[C@H](O)Cn2c(C(C)(C)CO)cc3cc(NC(=O)C4(c5ccc6c(c5)OC(F)(F)O6)CC4)c(F)cc32)CC1)c1ccc2c(N)c(C(=O)N[C@H]3CCc4cc(N5C[C@H]6CC[C@@H](C5)N6C(=O)OC(C)(C)C)ccc4C3)sc2n1]

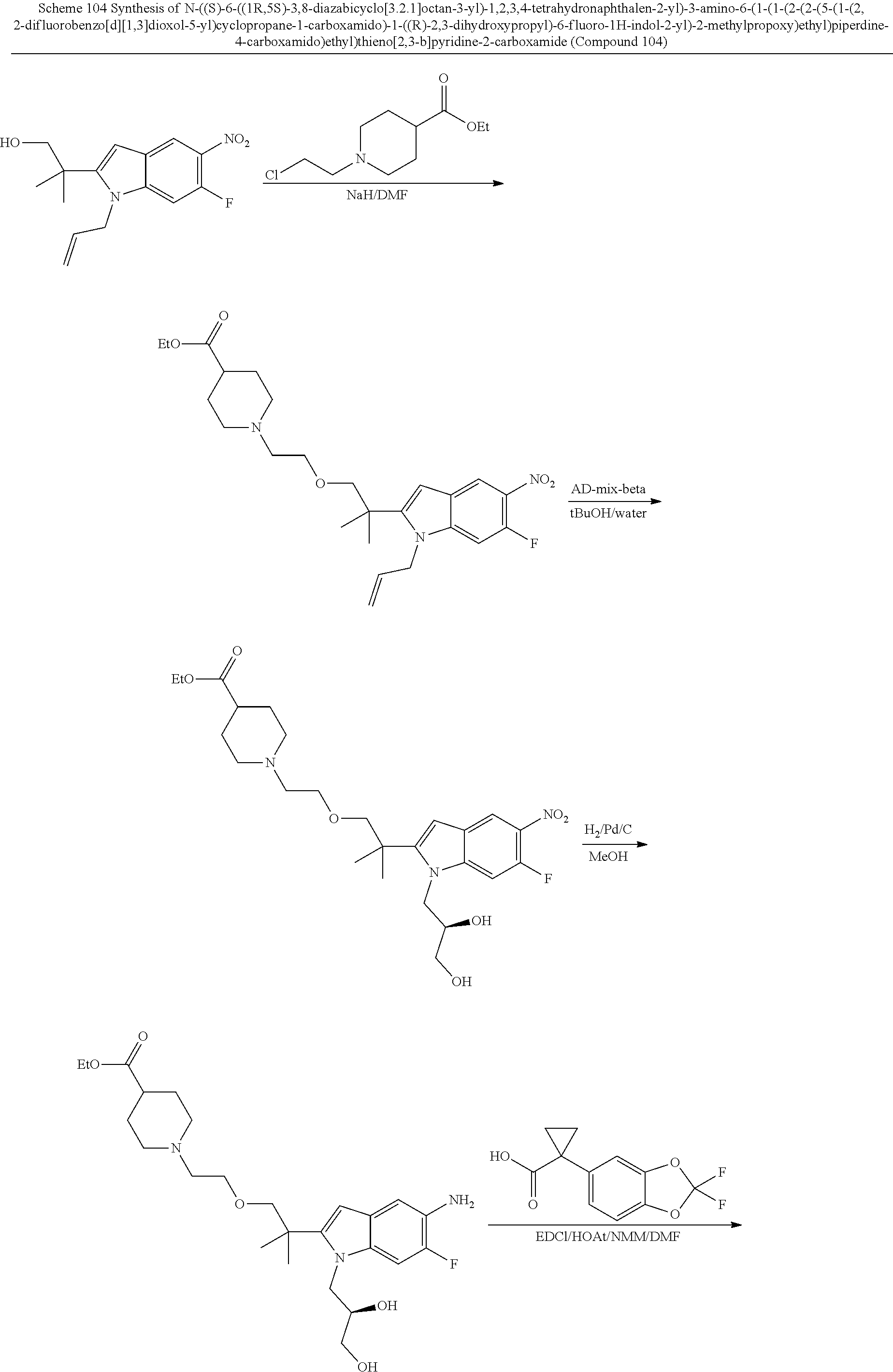

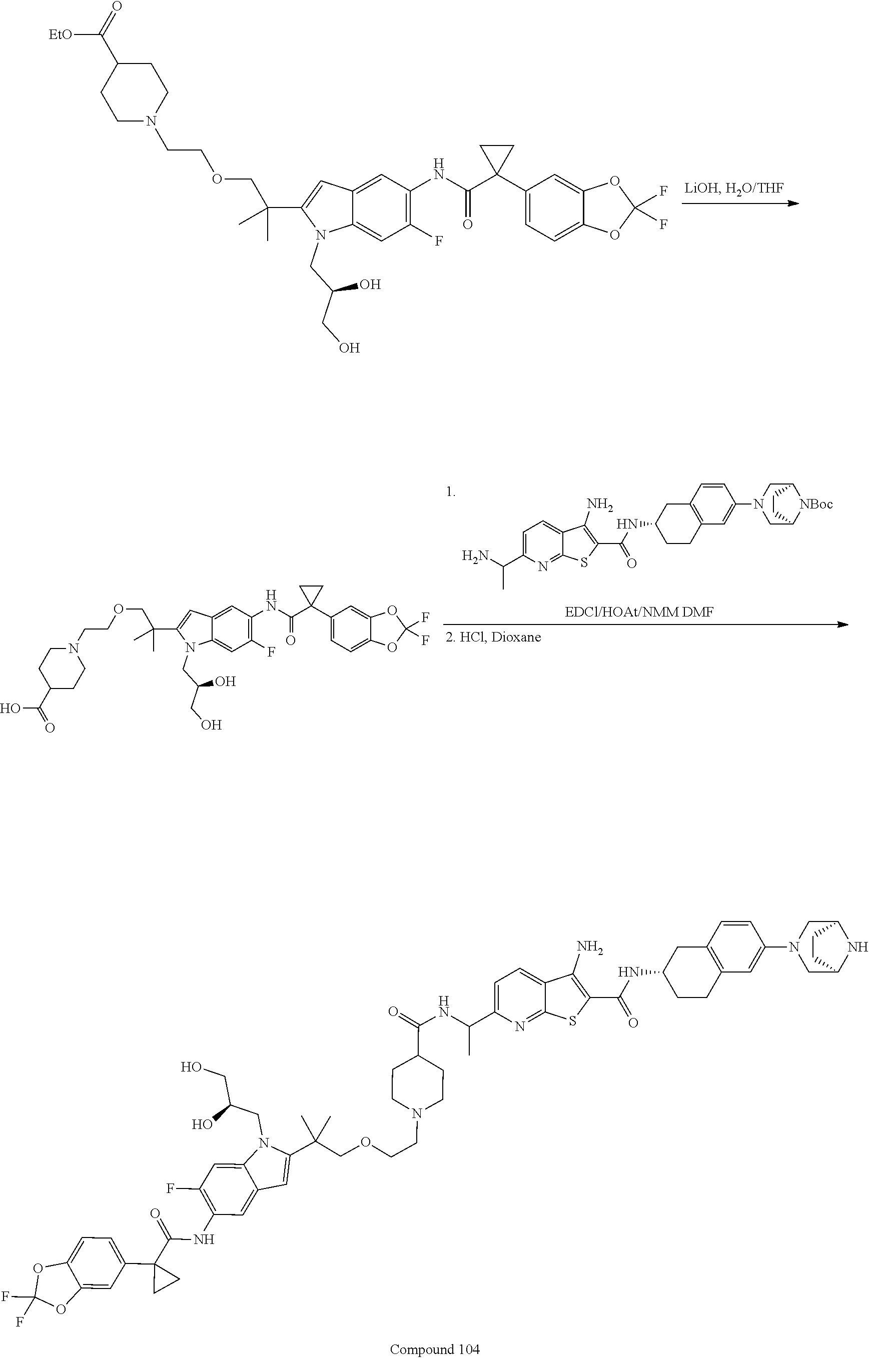 [SMILES: CCOC(=O)C1CCN(CCOCC(C)(C)c2cc3cc(NC(=O)C4(c5ccc6c(c5)OC(F)(F)O6)CC4)c(F)cc3n2C[C@@H](O)CO)CC1 ; CC(C)(COCCN1CCC(C(=O)O)CC1)c1cc2cc(NC(=O)C3(c4ccc5c(c4)OC(F)(F)O5)CC3)c(F)cc2n1C[C@@H](O)CO ; CC(N)c1ccc2c(N)c(C(=O)N[C@H]3CCc4cc(N5C[C@H]6CC[C@@H](C5)N6C(=O)OC(C)(C)C)ccc4C3)sc2n1 ; CC(NC(=O)C1CCN(CCOCC(C)(C)c2cc3cc(NC(=O)C4(c5ccc6c(c5)OC(F)(F)O6)CC4)c(F)cc3n2C[C@@H](O)CO)CC1)c1ccc2c(N)c(C(=O)N[C@H]3CCc4cc(N5C[C@H]6CC[C@@H](C5)N6)ccc4C3)sc2n1]

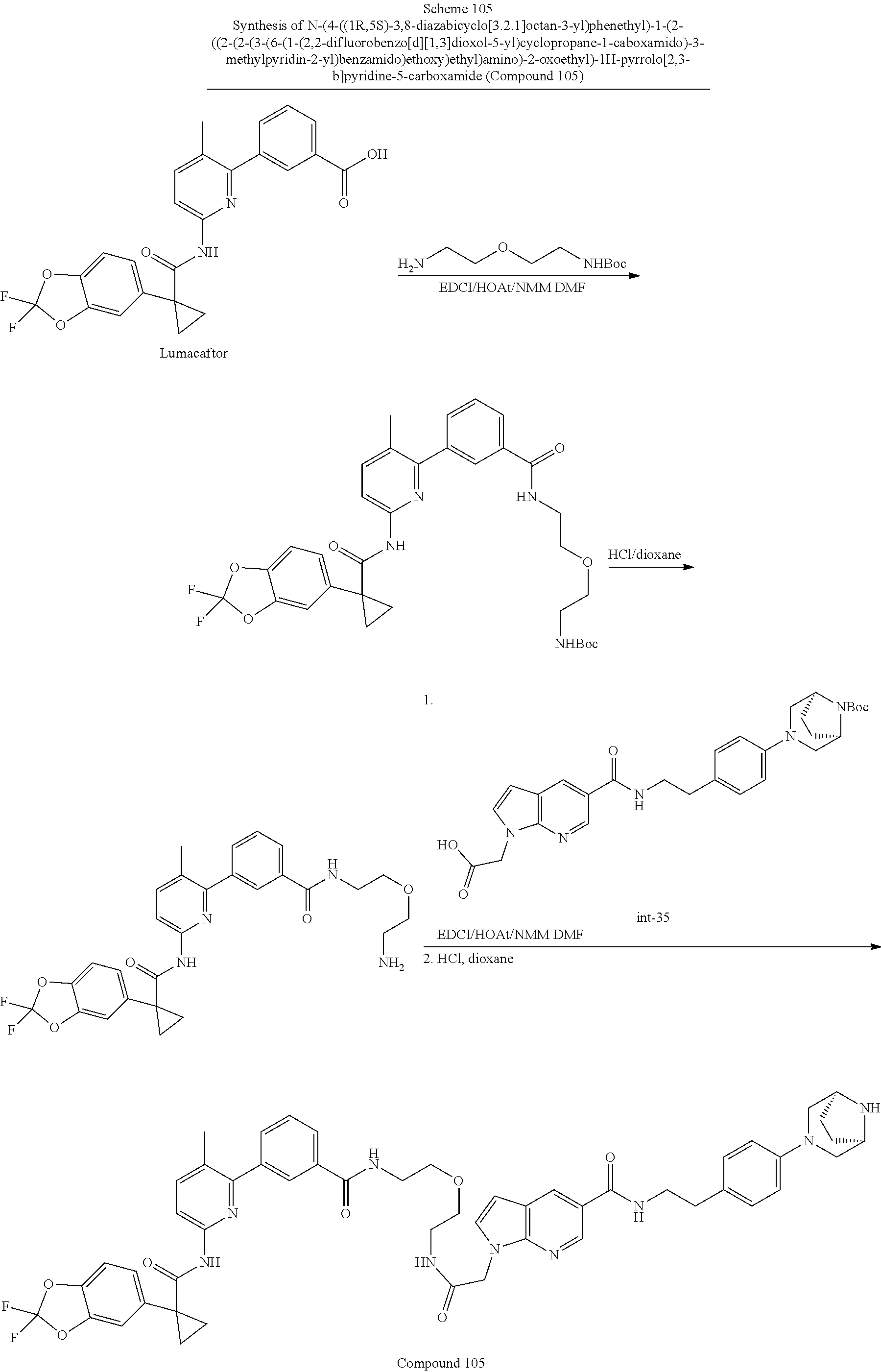

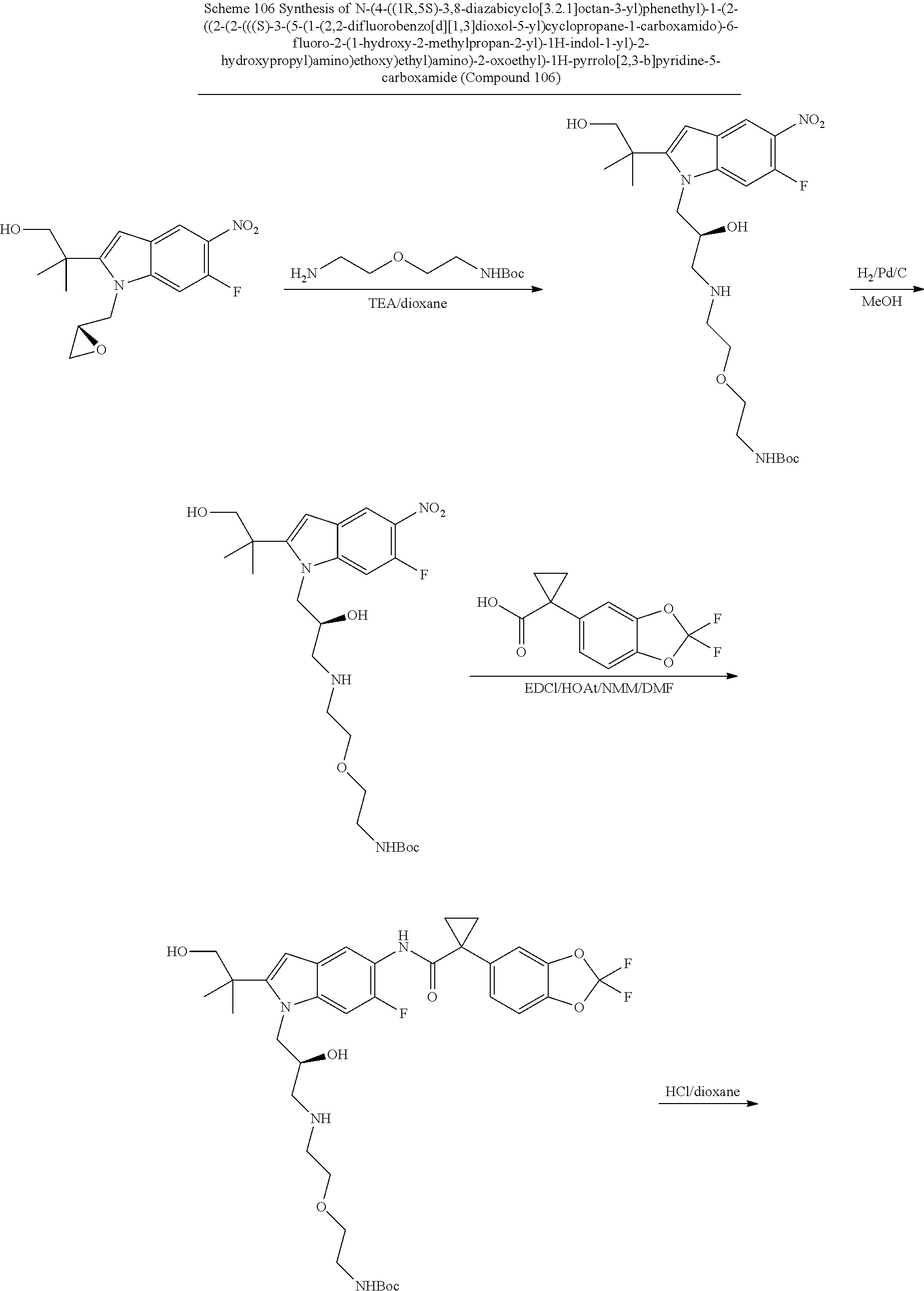

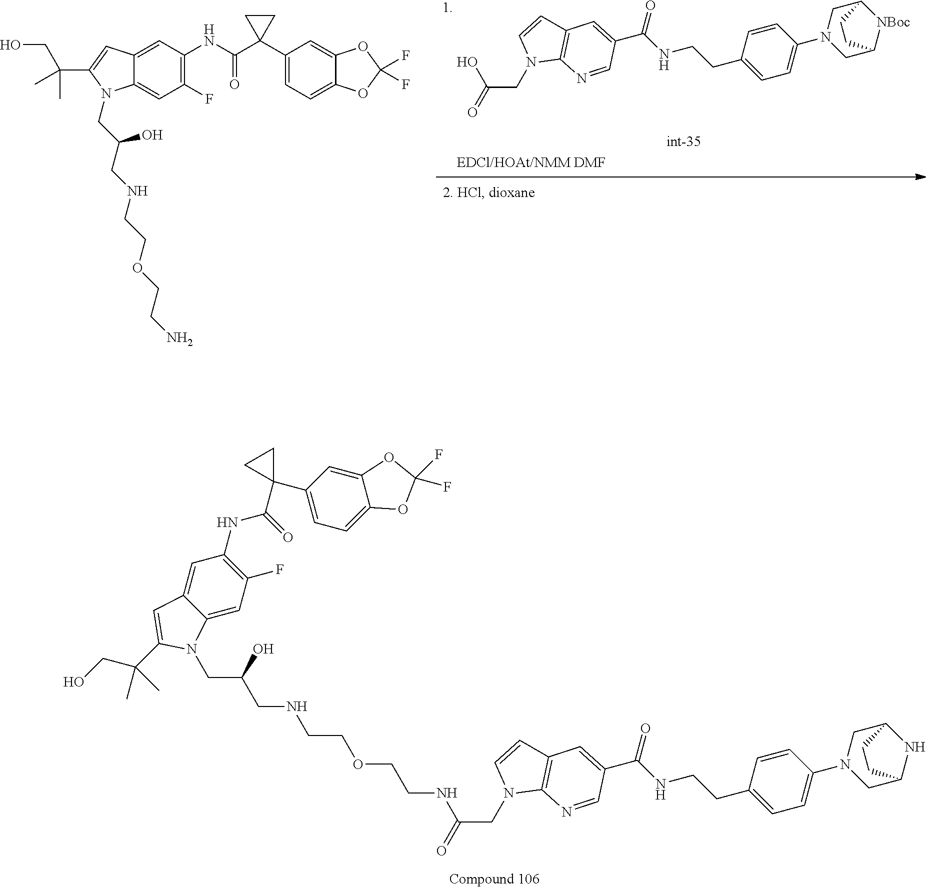 [SMILES: CC(C)(CO)c1cc2cc(NC(=O)C3(c4ccc5c(c4)OC(F)(F)O5)CC3)c(F)cc2n1C[C@@H](O)CNCCOCCN ; "1.    EDCl/HOAt/NMM DMF" ; CC(C)(C)OC(=O)N1[C@@H]2CC[C@H]1CN(c1ccc(CCNC(=O)c3cnc4c(ccn4CC(=O)O)c3)cc1)C2 ; CC(C)(CO)c1cc2cc(NC(=O)C3(c4ccc5c(c4)OC(F)(F)O5)CC3)c(F)cc2n1C[C@@H](O)CNCCOCCNC(=O)Cn1ccc2cc(C(=O)NCCc3ccc(N4C[C@H]5CC[C@@H](C4)N5)cc3)cnc21]

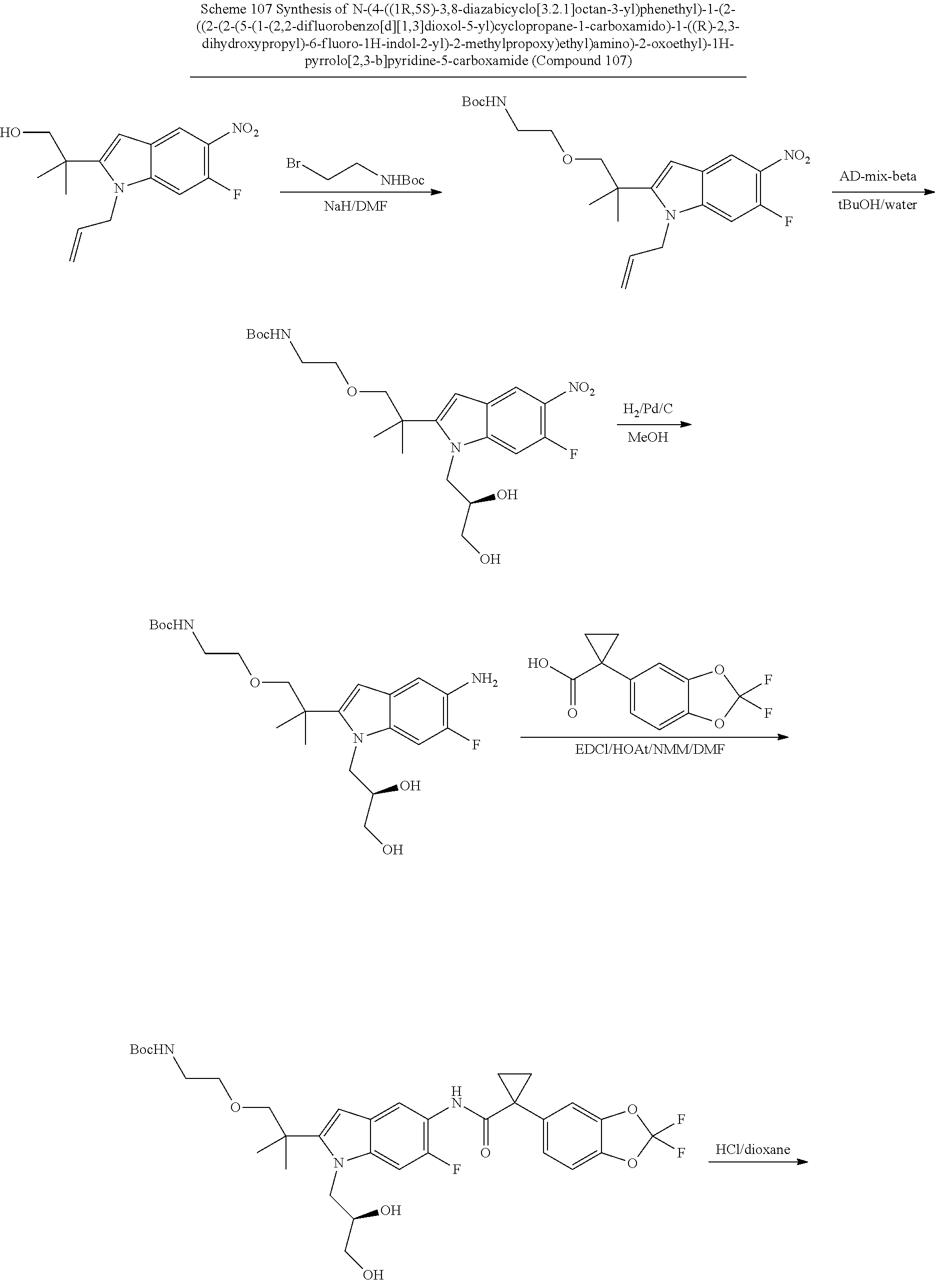

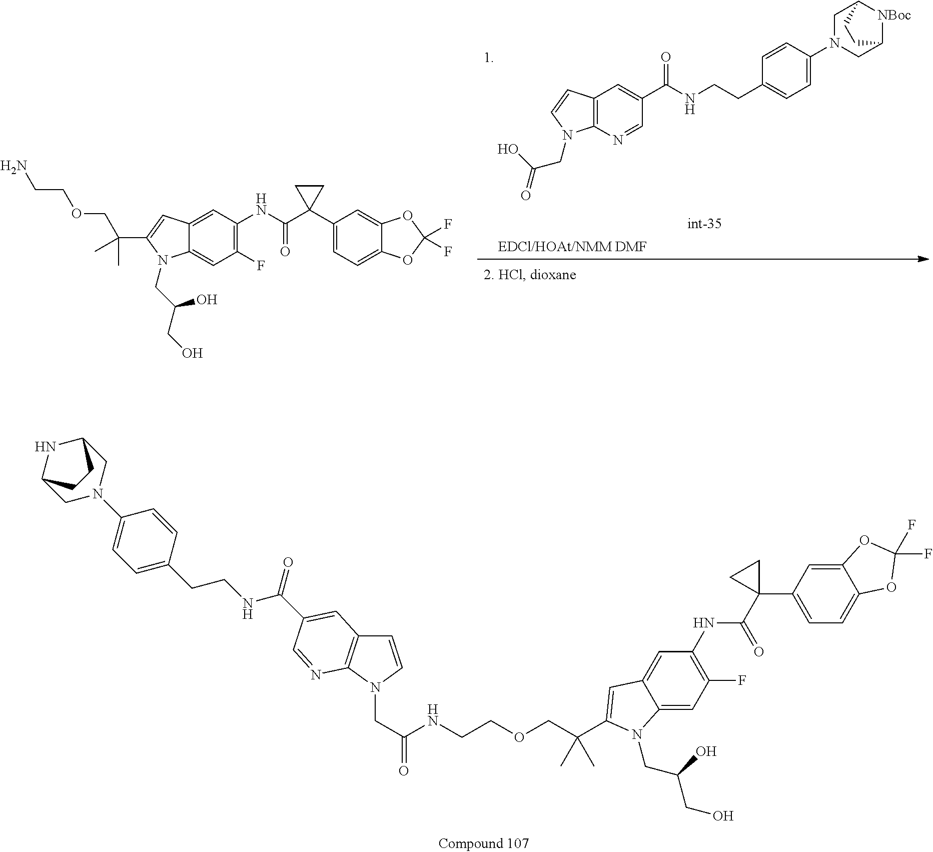 [SMILES: CC(C)(COCCN)c1cc2cc(NC(=O)C3(c4ccc5c(c4)OC(F)(F)O5)CC3)c(F)cc2n1C[C@@H](O)CO ; "1.    EDCl/HOAt/NMM DMF" ; CC(C)(C)OC(=O)N1[C@@H]2CC[C@H]1CN(c1ccc(CCNC(=O)c3cnc4c(ccn4CC(=O)O)c3)cc1)C2 ; CC(C)(COCCNC(=O)Cn1ccc2cc(C(=O)NCCc3ccc(N4C[C@H]5CC[C@@H](C4)N5)cc3)cnc21)c1cc2cc(NC(=O)C3(c4ccc5c(c4)OC(F)(F)O5)CC3)c(F)cc2n1C[C@@H](O)CO]

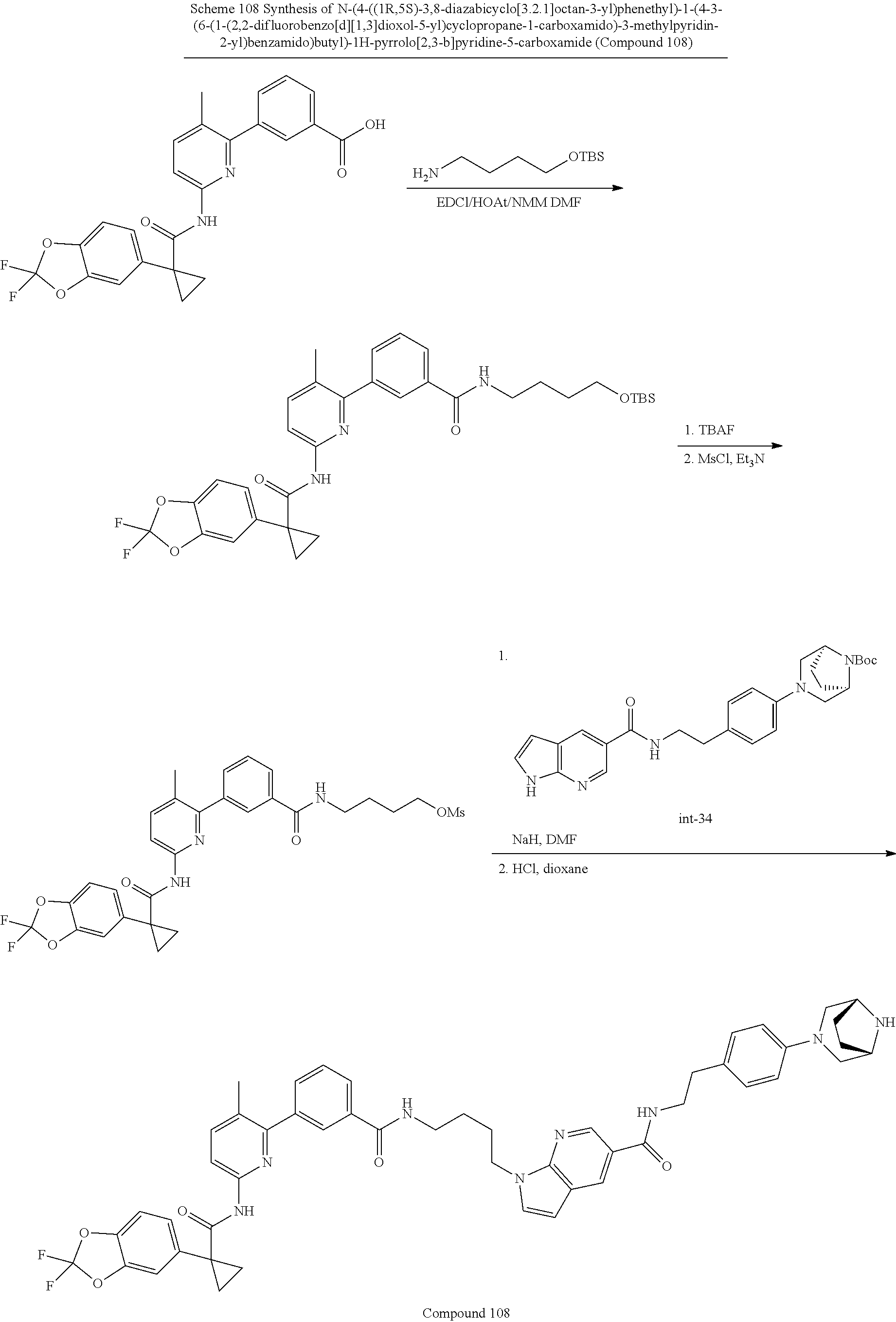

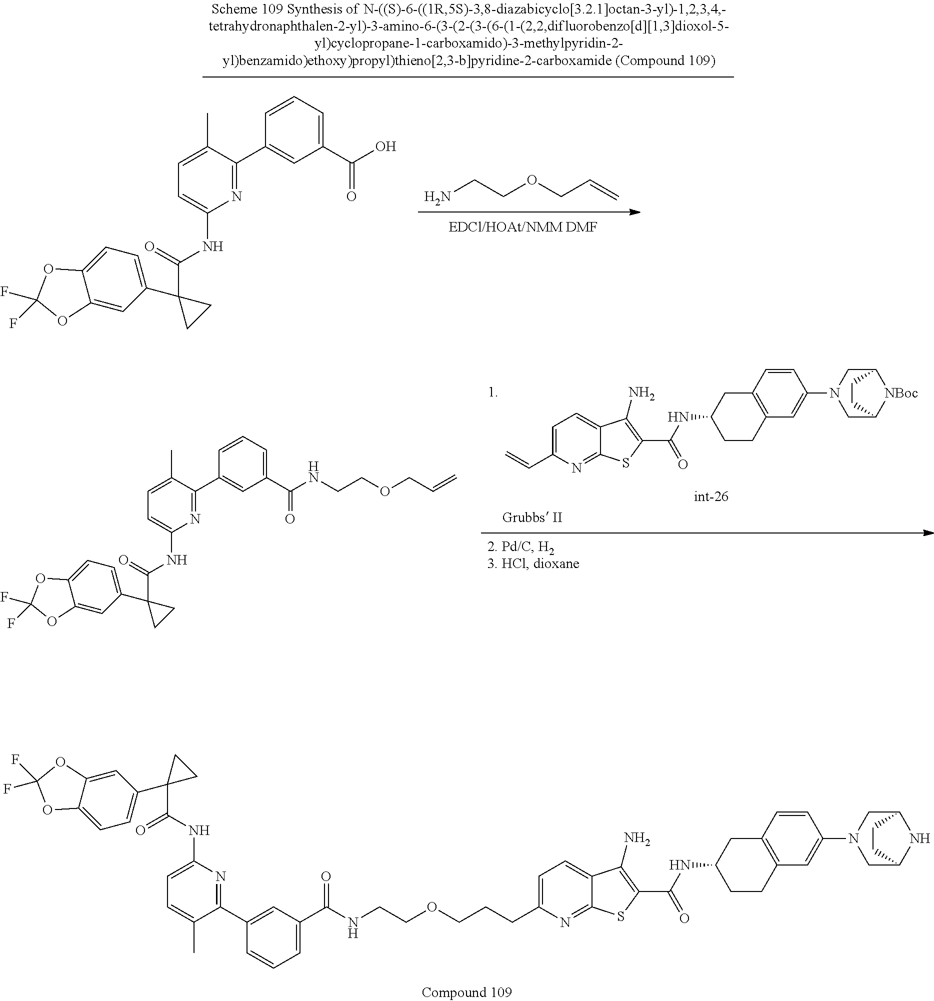

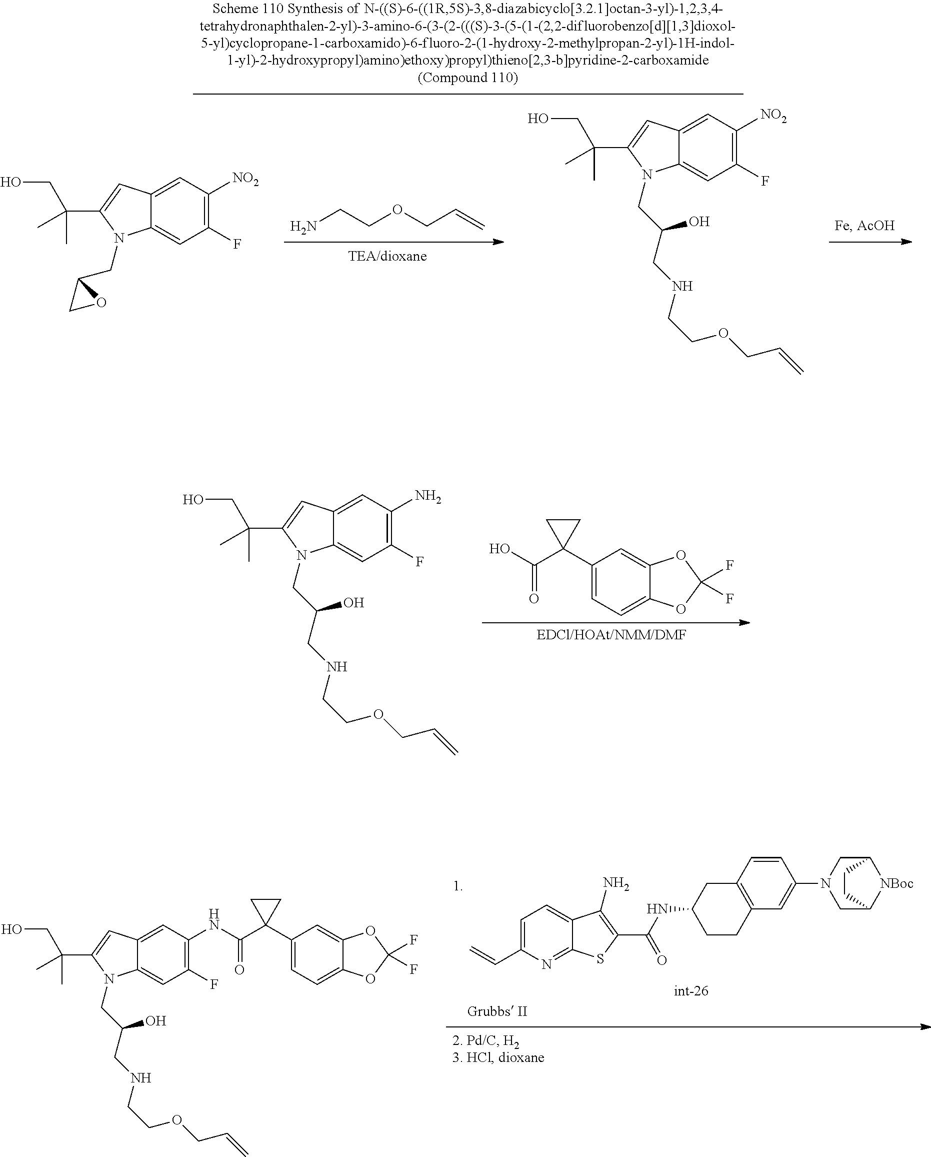

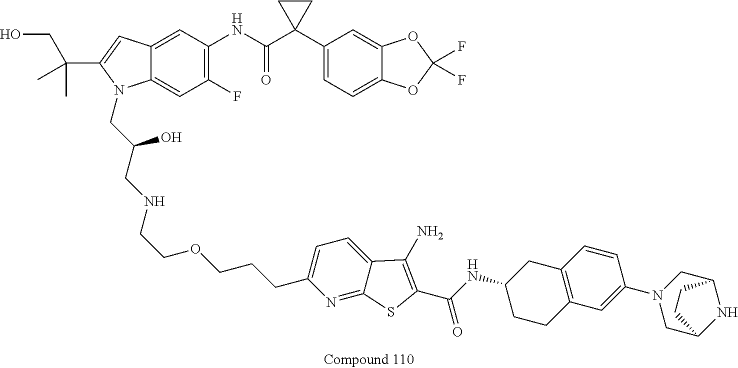 [SMILES: CC(C)(CO)c1cc2cc(NC(=O)C3(c4ccc5c(c4)OC(F)(F)O5)CC3)c(F)cc2n1C[C@@H](O)CNCCOCCCc1ccc2c(N)c(C(=O)N[C@H]3CCc4cc(N5C[C@H]6CC[C@@H](C5)N6)ccc4C3)sc2n1]

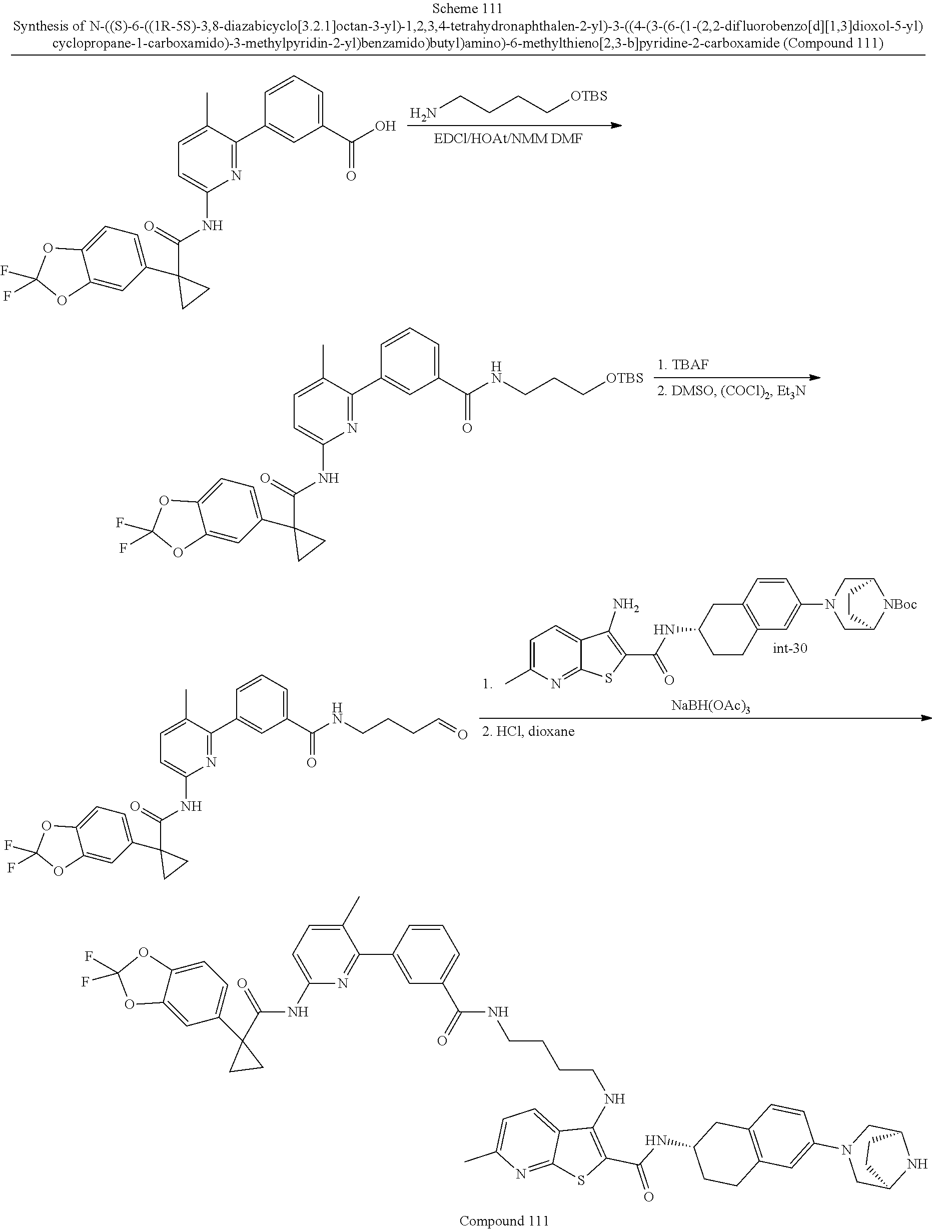

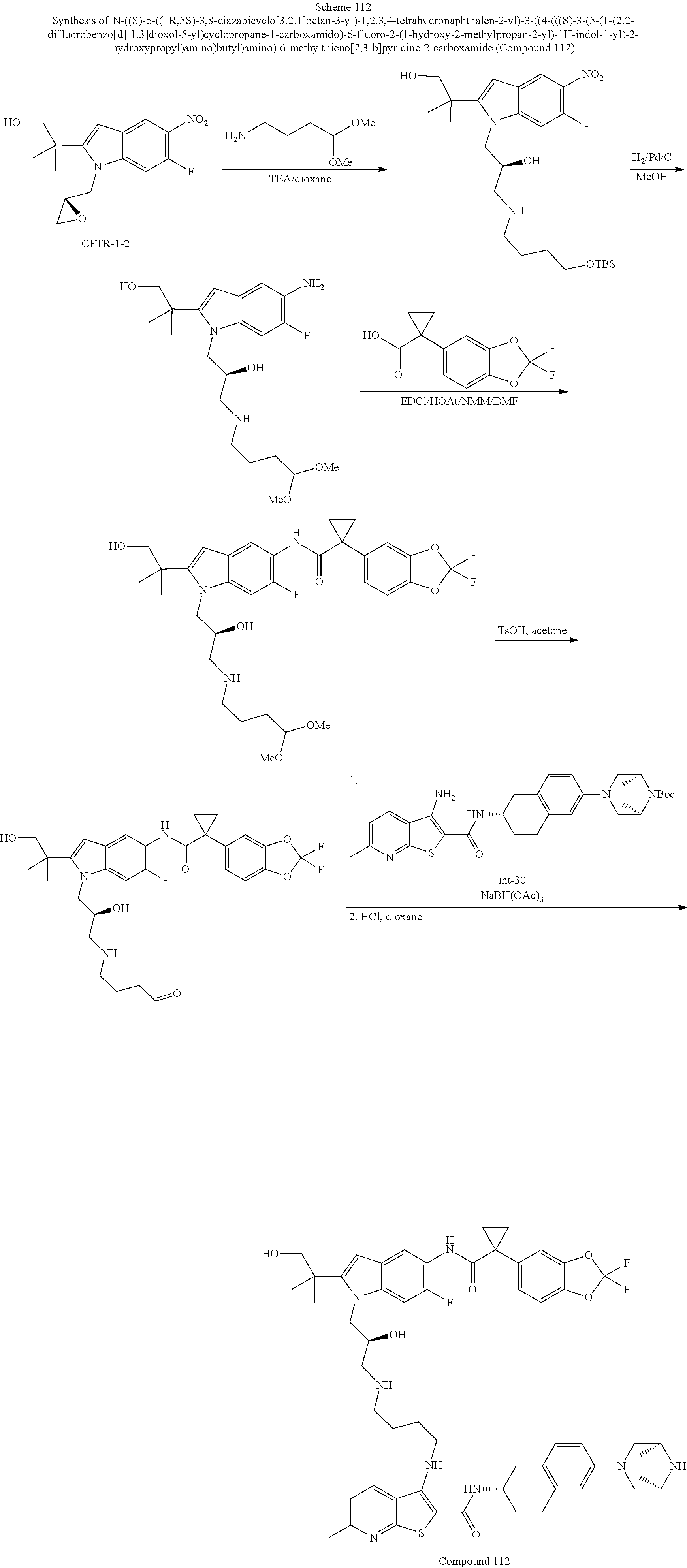

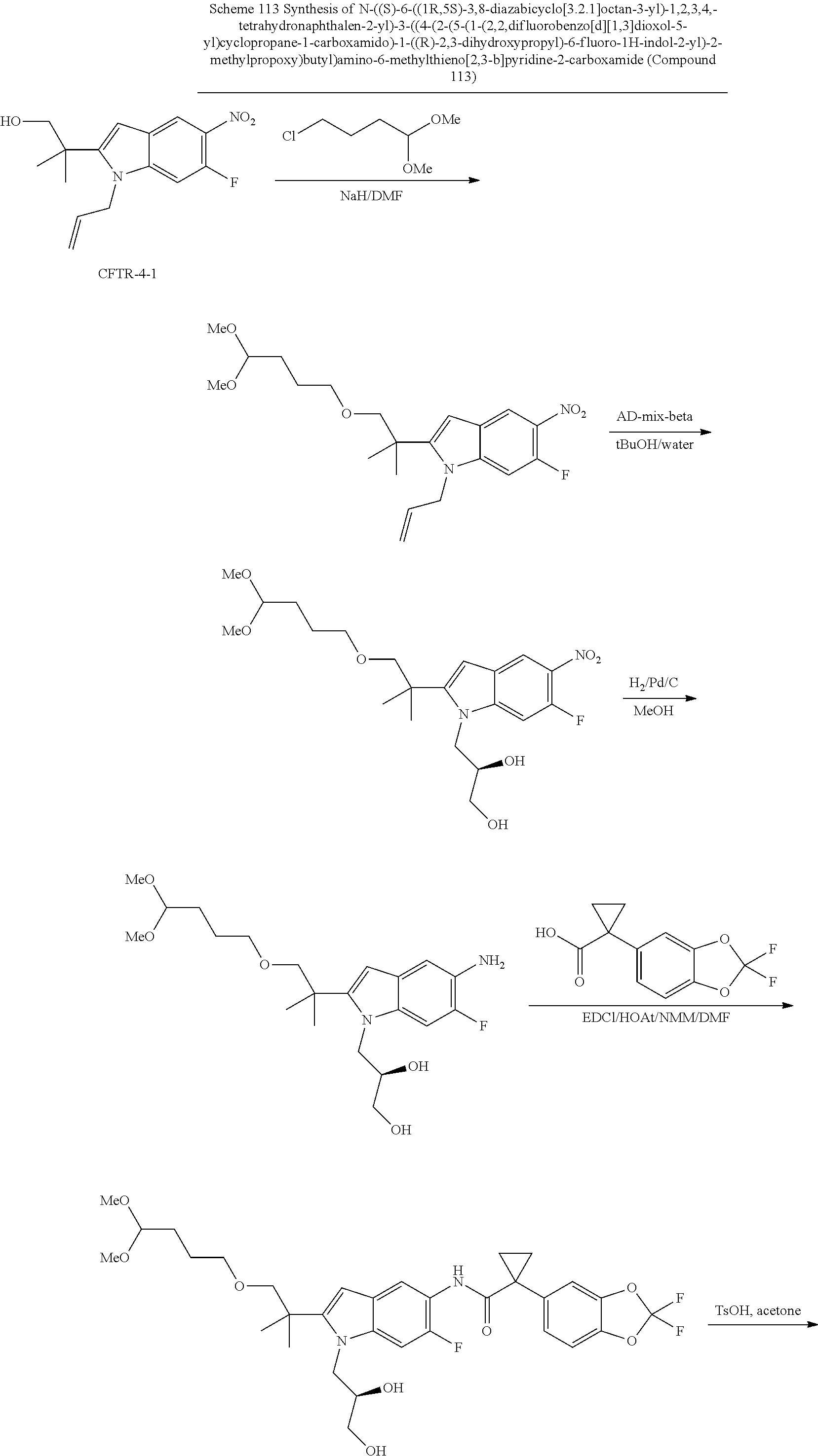

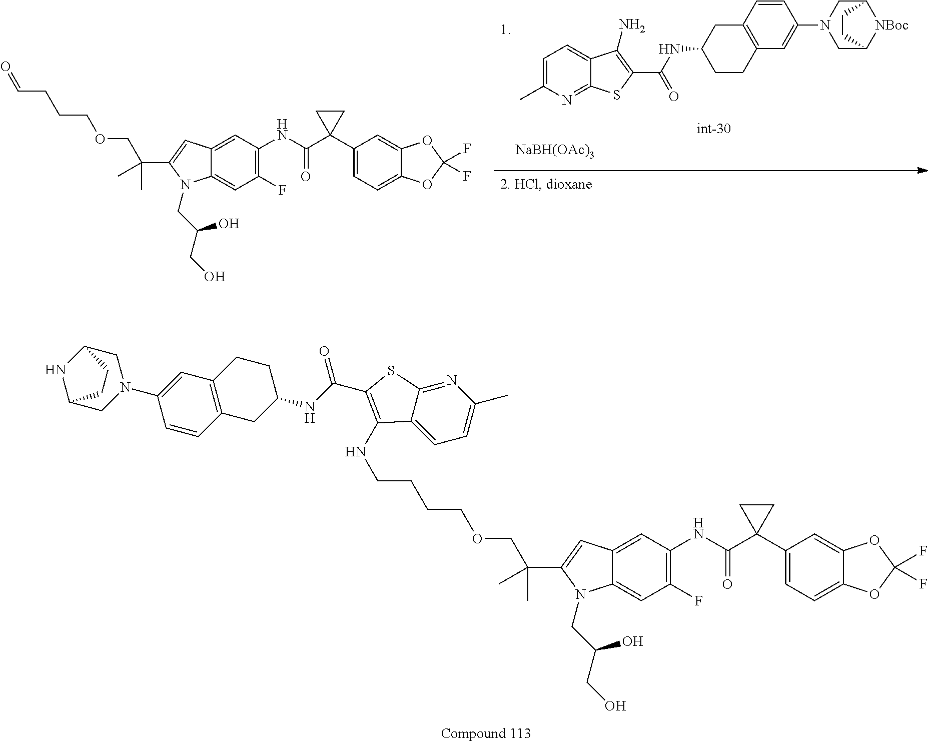

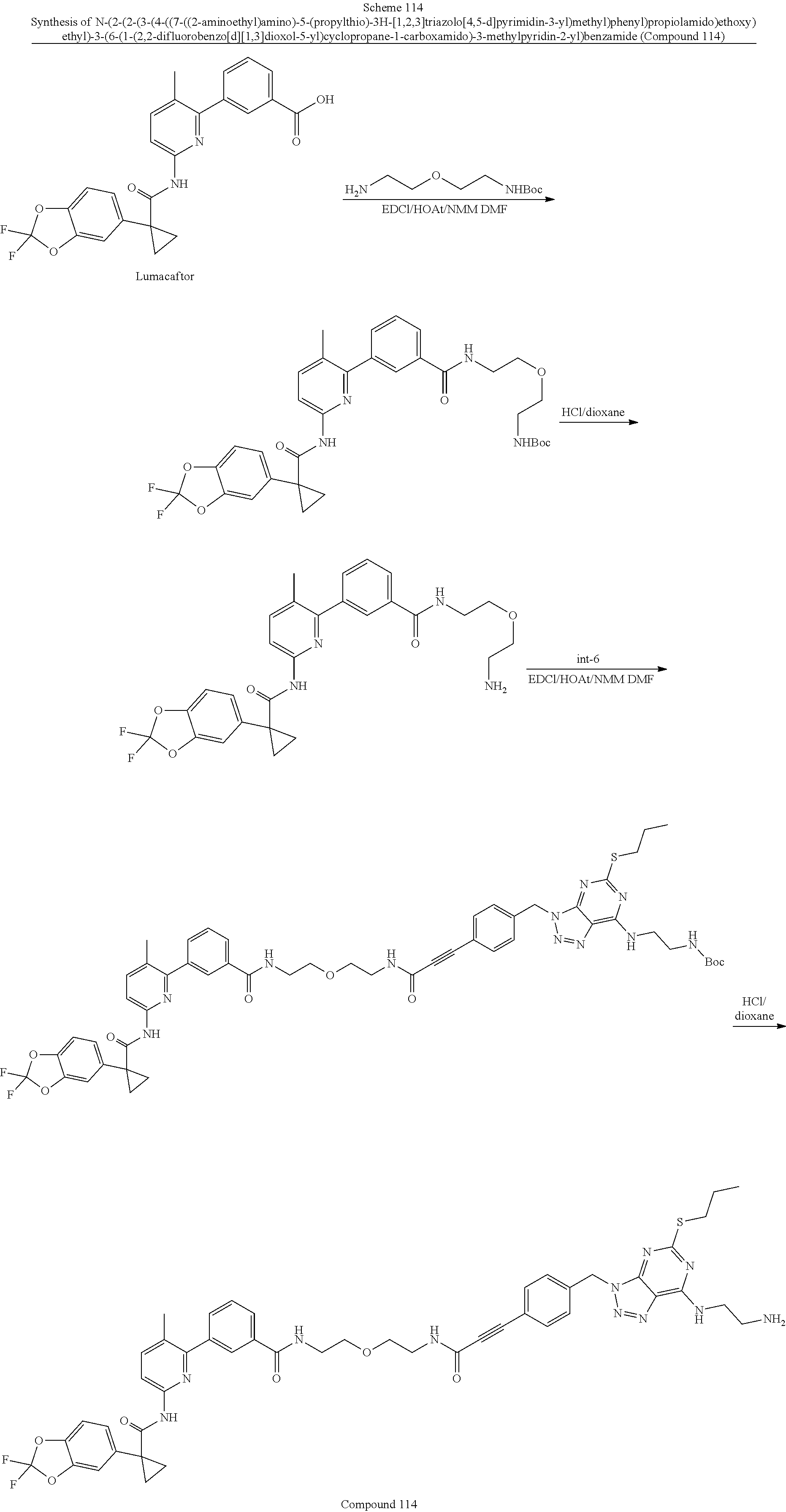 [SMILES: Cc1ccc(NC(=O)C2(c3ccc4c(c3)OC(F)(F)O4)CC2)nc1-c1cccc(C(=O)O)c1 ; CC(C)(C)OC(=O)NCCOCCN ; Cc1ccc(NC(=O)C2(c3ccc4c(c3)OC(F)(F)O4)CC2)nc1-c1cccc(C(=O)NCCOCCNC(=O)OC(C)(C)C)c1 ; Cc1ccc(NC(=O)C2(c3ccc4c(c3)OC(F)(F)O4)CC2)nc1-c1cccc(C(=O)NCCOCCN)c1 ; CCCSc1nc(NCCNC(=O)OC(C)(C)C)c2nnn(Cc3ccc(C#CC(=O)NCCOCCNC(=O)c4cccc(-c5nc(NC(=O)C6(c7ccc8c(c7)OC(F)(F)O8)CC6)ccc5C)c4)cc3)c2n1 ; CCCSc1nc(NCCN)c2nnn(Cc3ccc(C#CC(=O)NCCOCCNC(=O)c4cccc(-c5nc(NC(=O)C6(c7ccc8c(c7)OC(F)(F)O8)CC6)ccc5C)c4)cc3)c2n1]

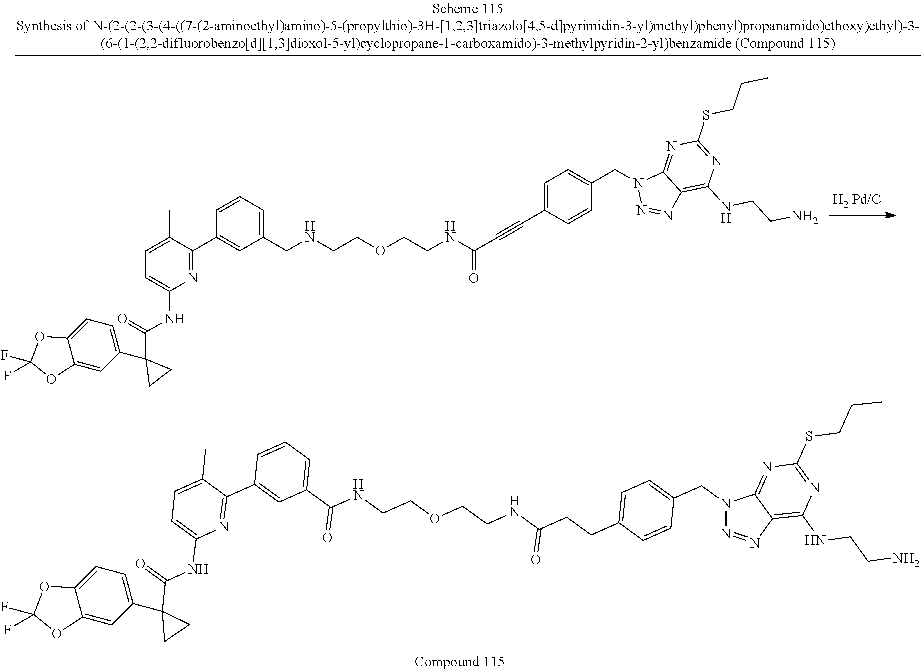

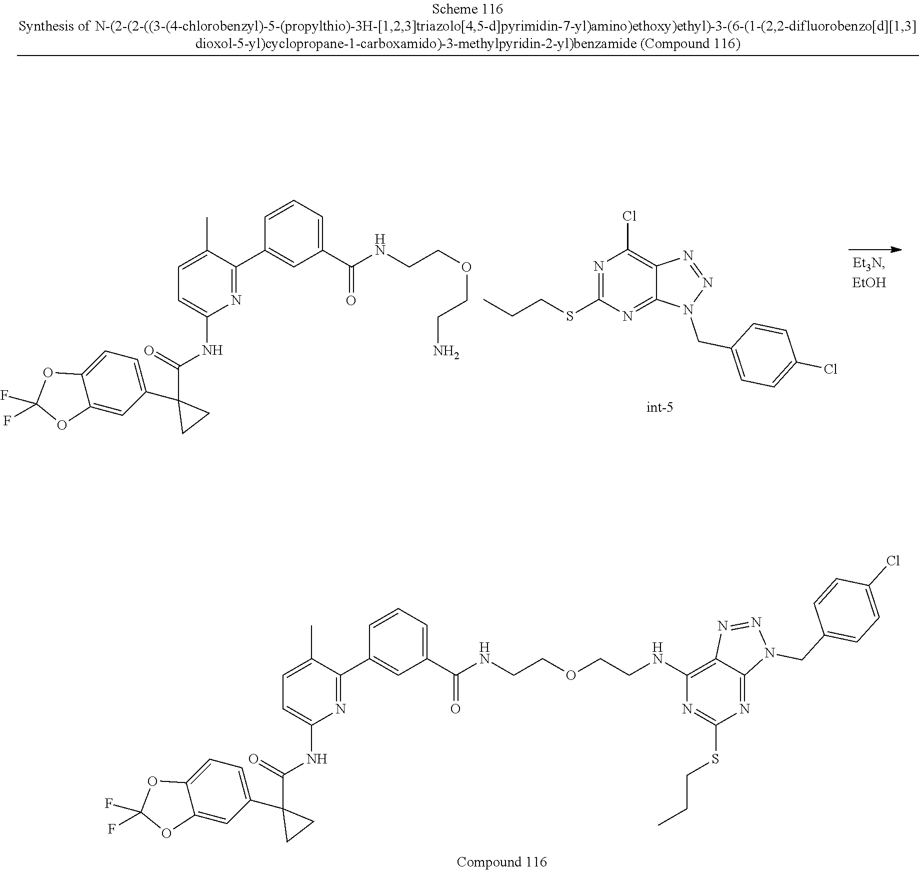

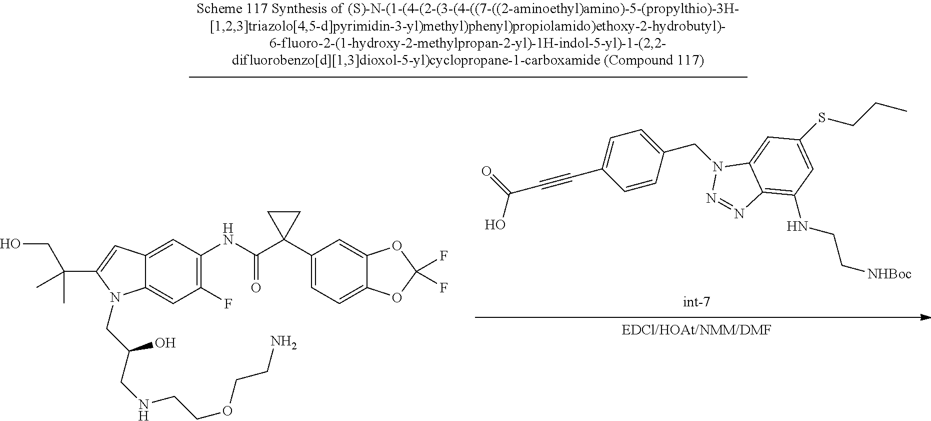 [SMILES: CC(C)(CO)c1cc2cc(NC(=O)C3(c4ccc5c(c4)OC(F)(F)O5)CC3)c(F)cc2n1C[C@@H](O)CNCCOCCN ; CCCSc1cc(NCCNC(=O)OC(C)(C)C)c2nnn(Cc3ccc(C#CC(=O)O)cc3)c2c1 ; "EDCl/HOAt/NMM/DMF"]

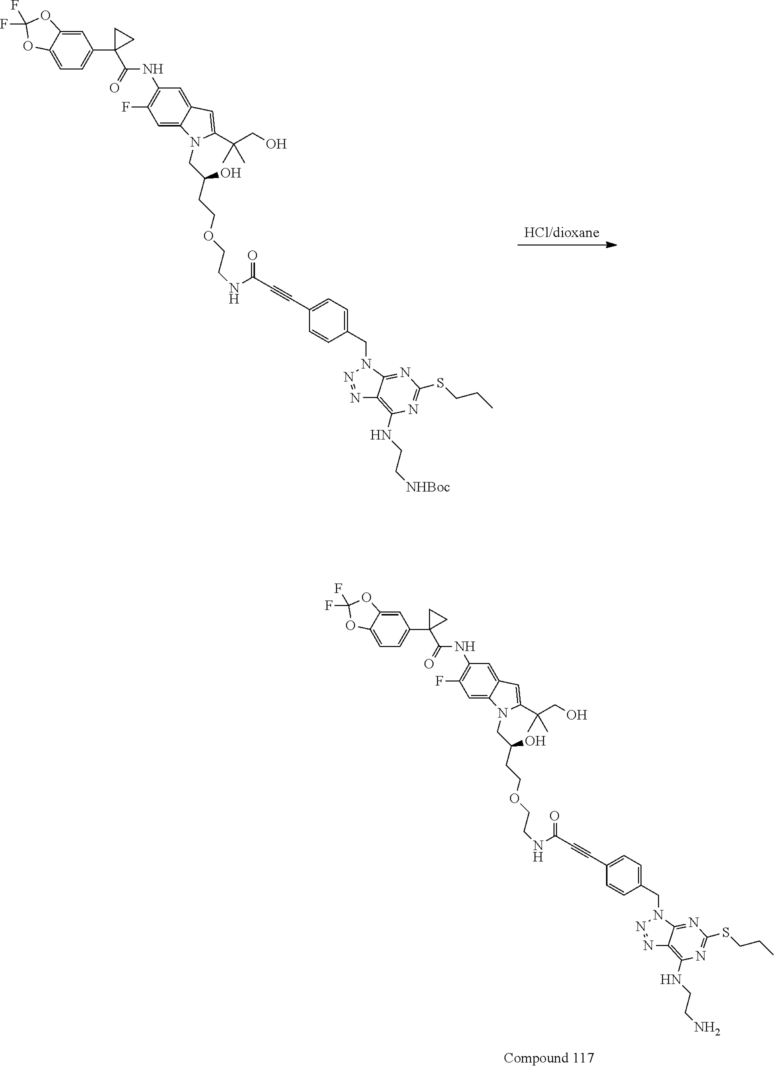 [SMILES: CCCSc1nc(NCCNC(=O)OC(C)(C)C)c2nnn(Cc3ccc(C#CC(=O)NCCOCC[C@H](O)Cn4c(C(C)(C)CO)cc5cc(NC(=O)C6(c7ccc8c(c7)OC(F)(F)O8)CC6)c(F)cc54)cc3)c2n1 ; "HCl/dioxane" ; CCCSc1nc(NCCN)c2nnn(Cc3ccc(C#CC(=O)NCCOCC[C@H](O)Cn4c(C(C)(C)CO)cc5cc(NC(=O)C6(c7ccc8c(c7)OC(F)(F)O8)CC6)c(F)cc54)cc3)c2n1]

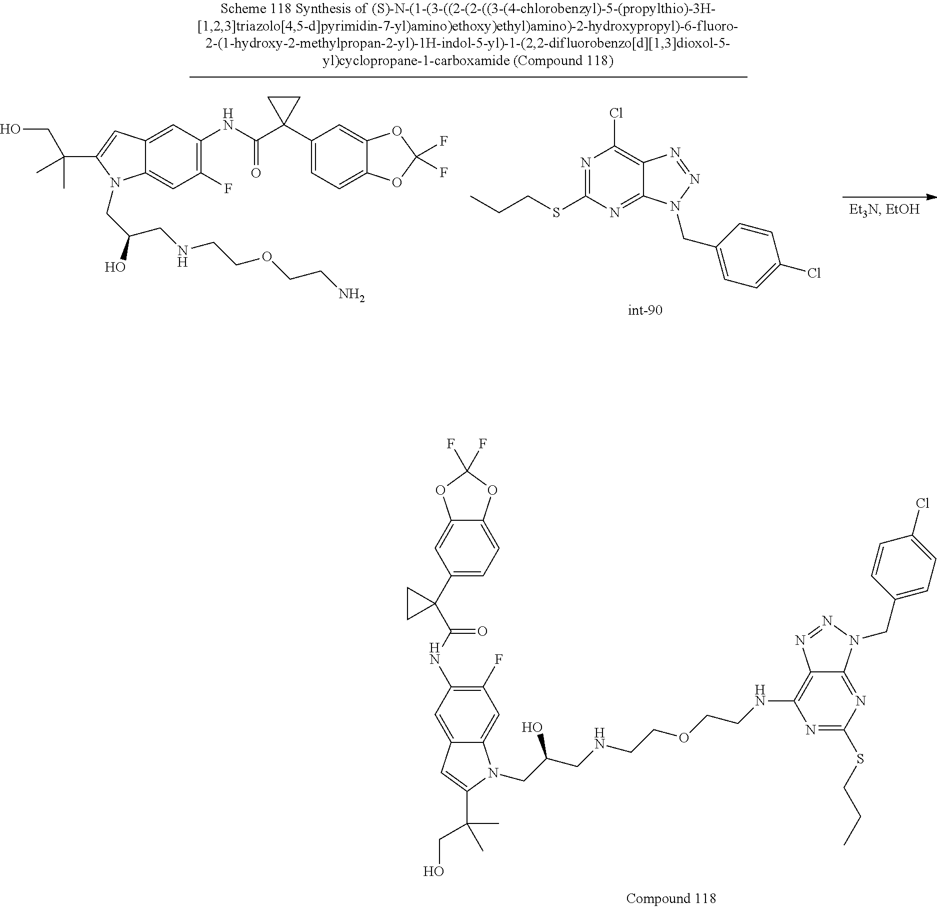

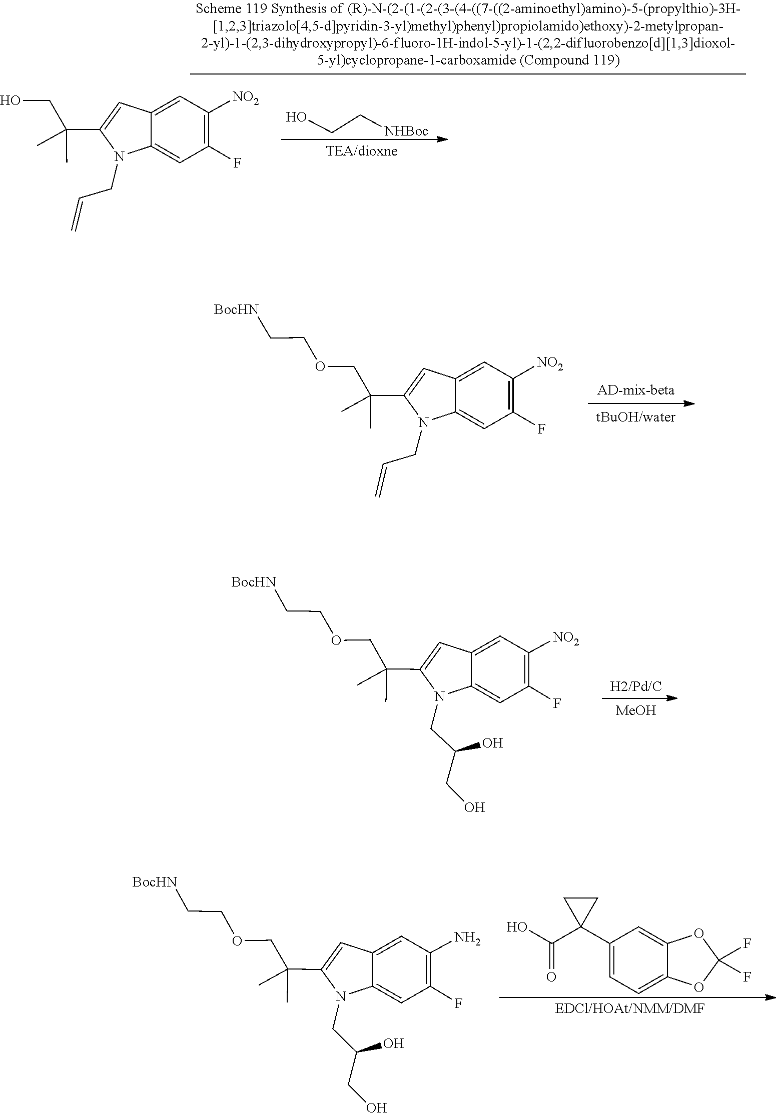

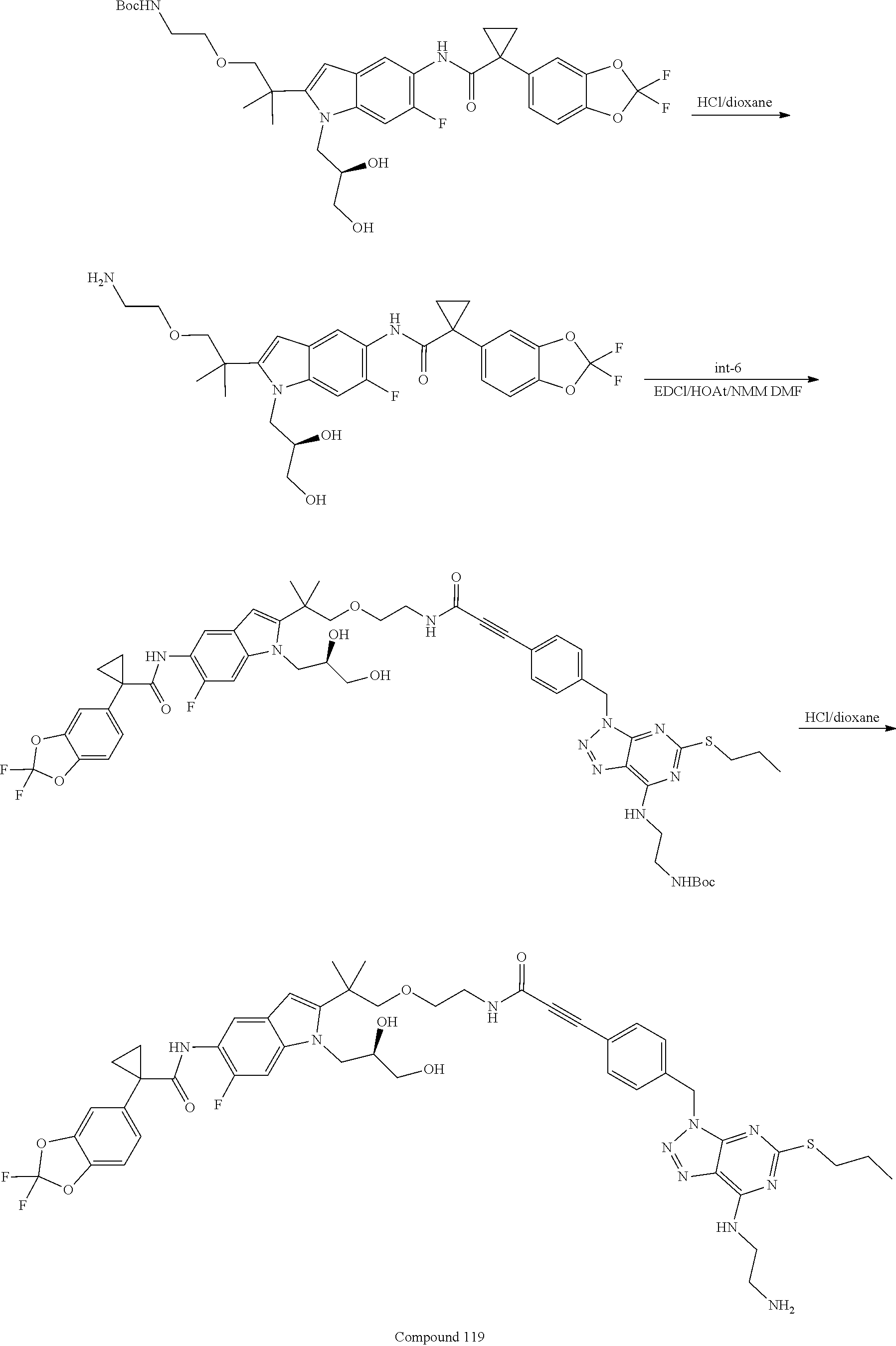 [SMILES: CC(C)(C)OC(=O)NCCOCC(C)(C)c1cc2cc(NC(=O)C3(c4ccc5c(c4)OC(F)(F)O5)CC3)c(F)cc2n1C[C@@H](O)CO ; CC(C)(COCCN)c1cc2cc(NC(=O)C3(c4ccc5c(c4)OC(F)(F)O5)CC3)c(F)cc2n1C[C@@H](O)CO ; CCCSc1nc(NCCNC(=O)OC(C)(C)C)c2nnn(Cc3ccc(C#CC(=O)NCCOCC(C)(C)c4cc5cc(NC(=O)C6(c7ccc8c(c7)OC(F)(F)O8)CC6)c(F)cc5n4C[C@@H](O)CO)cc3)c2n1 ; CCCSc1nc(NCCN)c2nnn(Cc3ccc(C#CC(=O)NCCOCC(C)(C)c4cc5cc(NC(=O)C6(c7ccc8c(c7)OC(F)(F)O8)CC6)c(F)cc5n4C[C@@H](O)CO)cc3)c2n1]

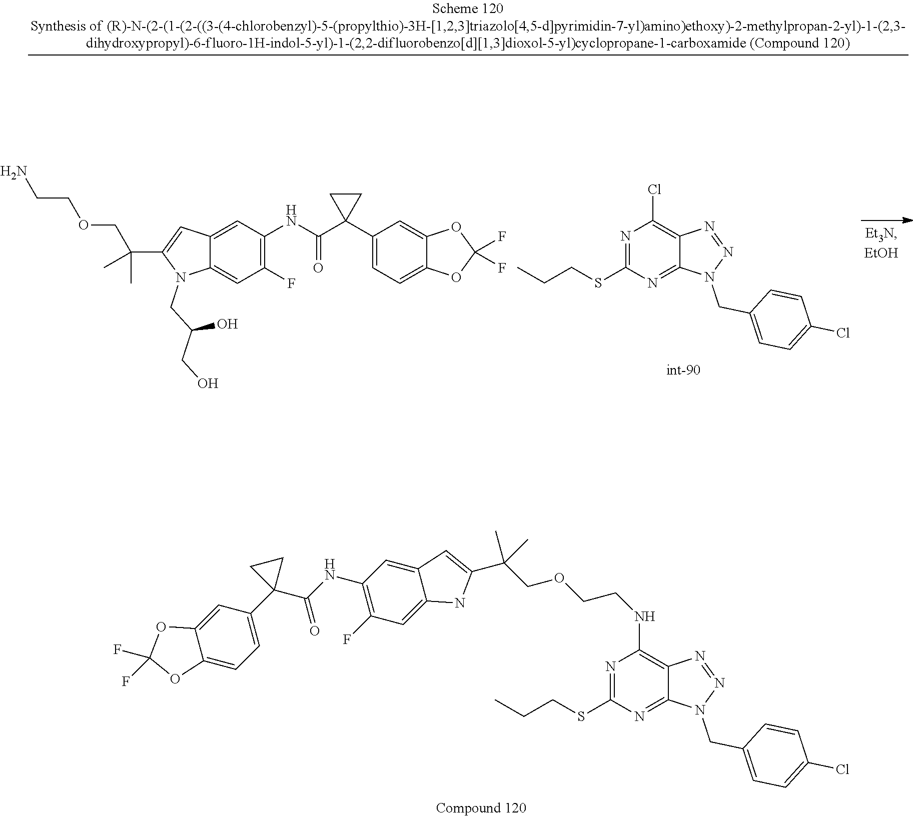

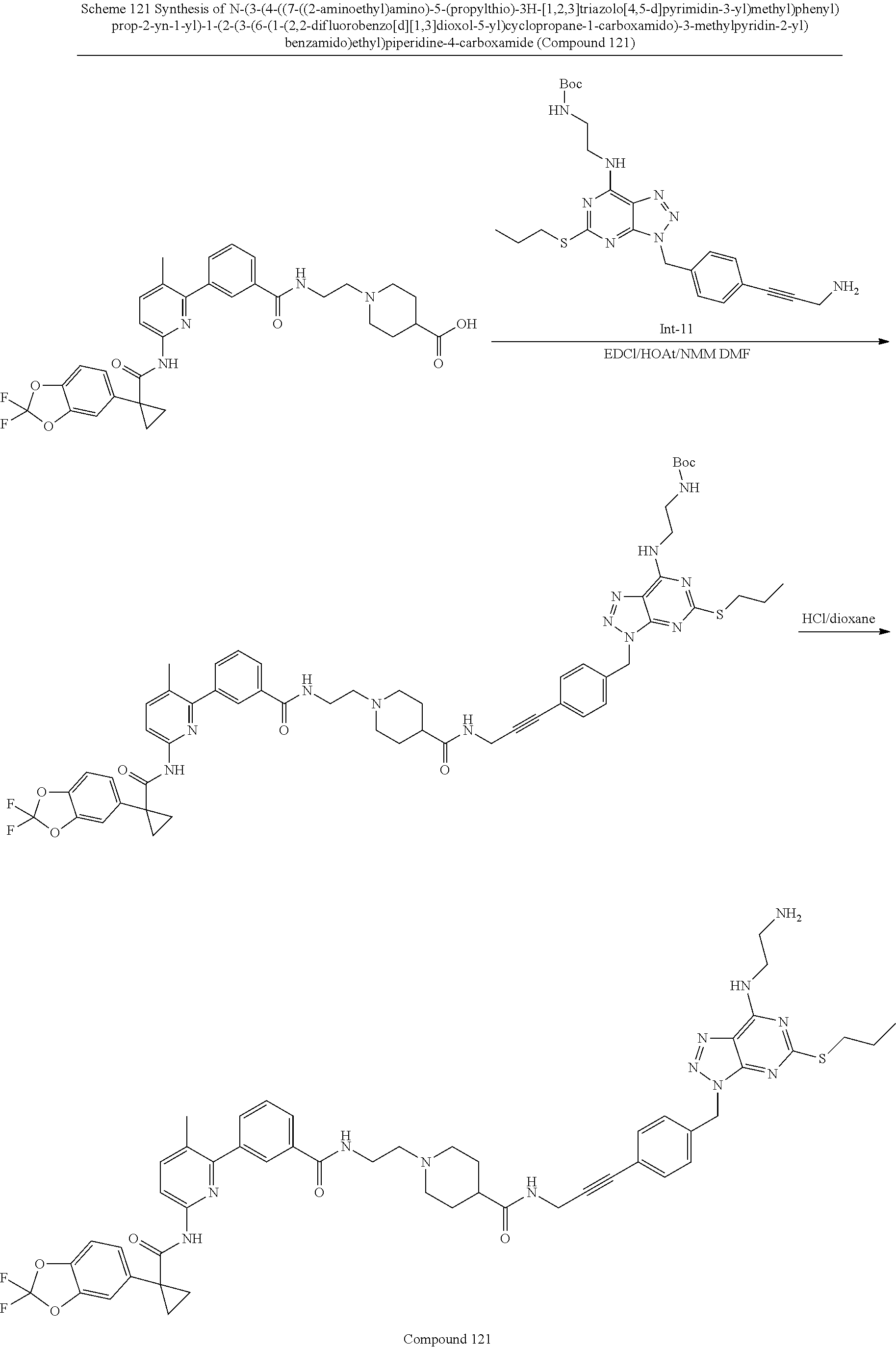 [SMILES: Cc1ccc(NC(=O)C2(c3ccc4c(c3)OC(F)(F)O4)CC2)nc1-c1cccc(C(=O)NCCN2CCC(C(=O)O)CC2)c1 ; CCCSc1nc(NCCNC(=O)OC(C)(C)C)c2nnn(Cc3ccc(C#CCN)cc3)c2n1 ; CCCSc1nc(NCCNC(=O)OC(C)(C)C)c2nnn(Cc3ccc(C#CCNC(=O)C4CCN(CCNC(=O)c5cccc(-c6nc(NC(=O)C7(c8ccc9c(c8)OC(F)(F)O9)CC7)ccc6C)c5)CC4)cc3)c2n1 ; CCCSc1nc(NCCN)c2nnn(Cc3ccc(C#CCNC(=O)C4CCN(CCNC(=O)c5cccc(-c6nc(NC(=O)C7(c8ccc9c(c8)OC(F)(F)O9)CC7)ccc6C)c5)CC4)cc3)c2n1]

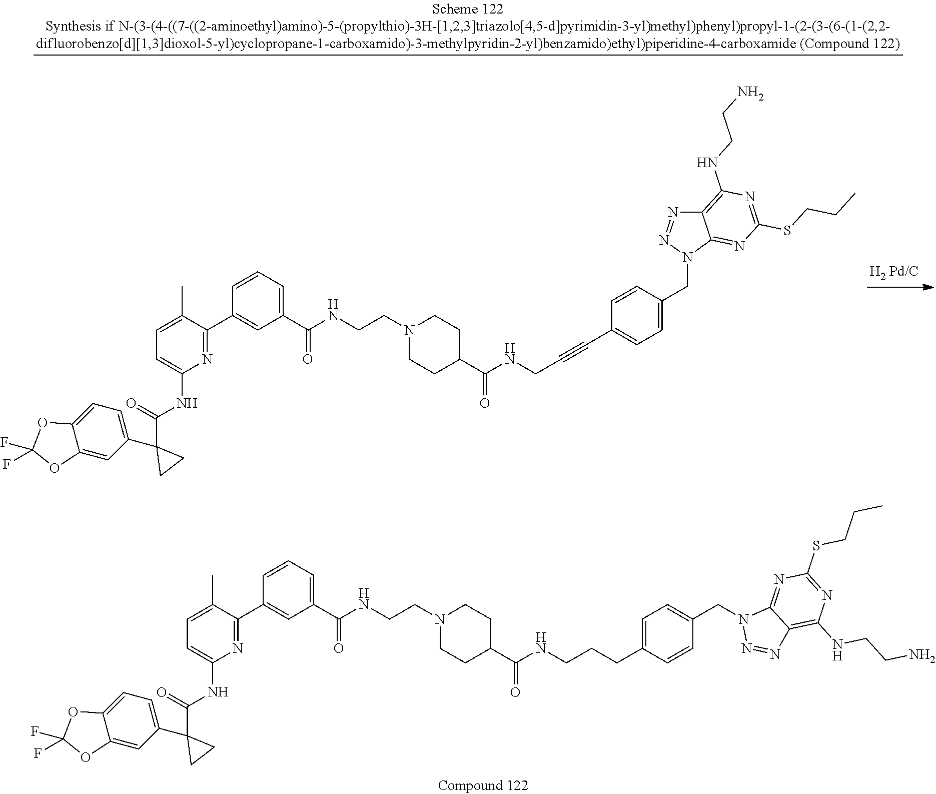

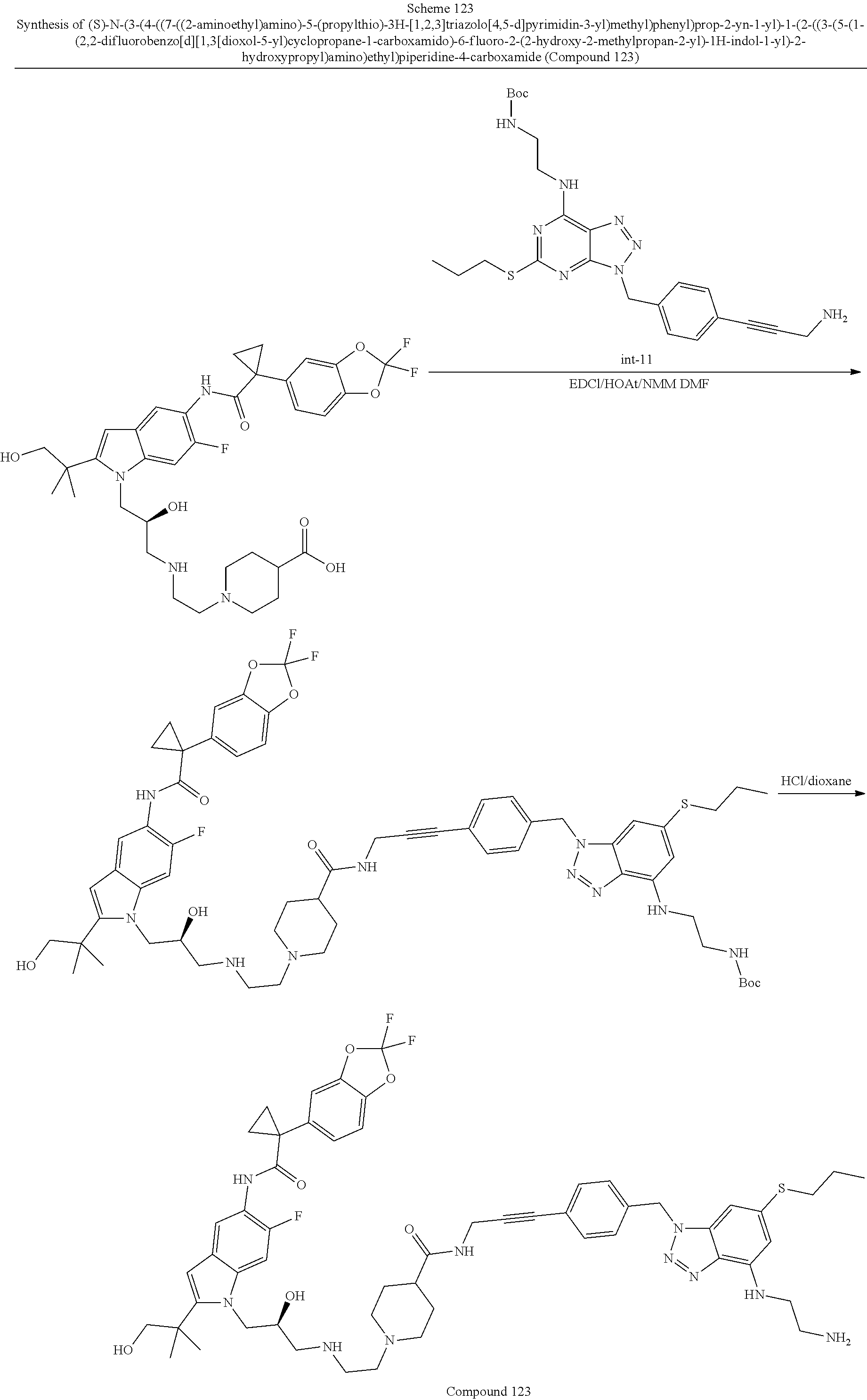

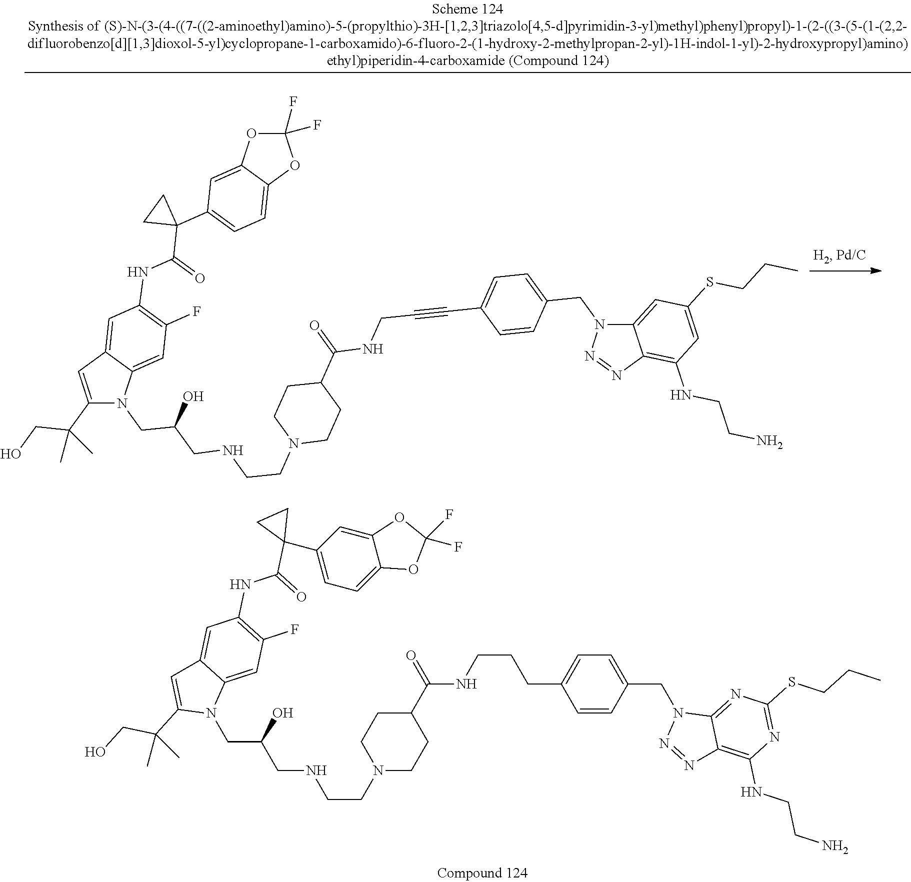

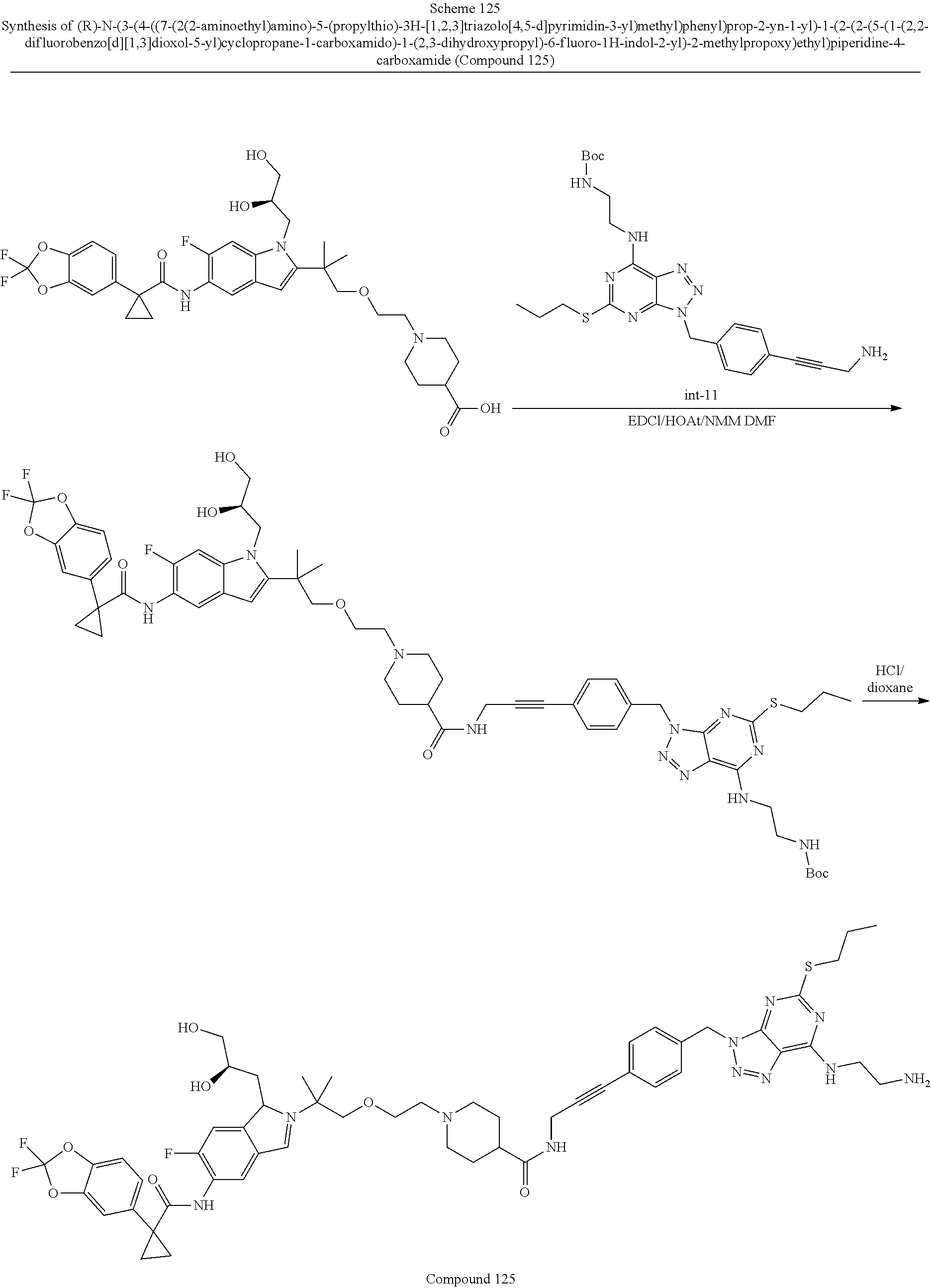

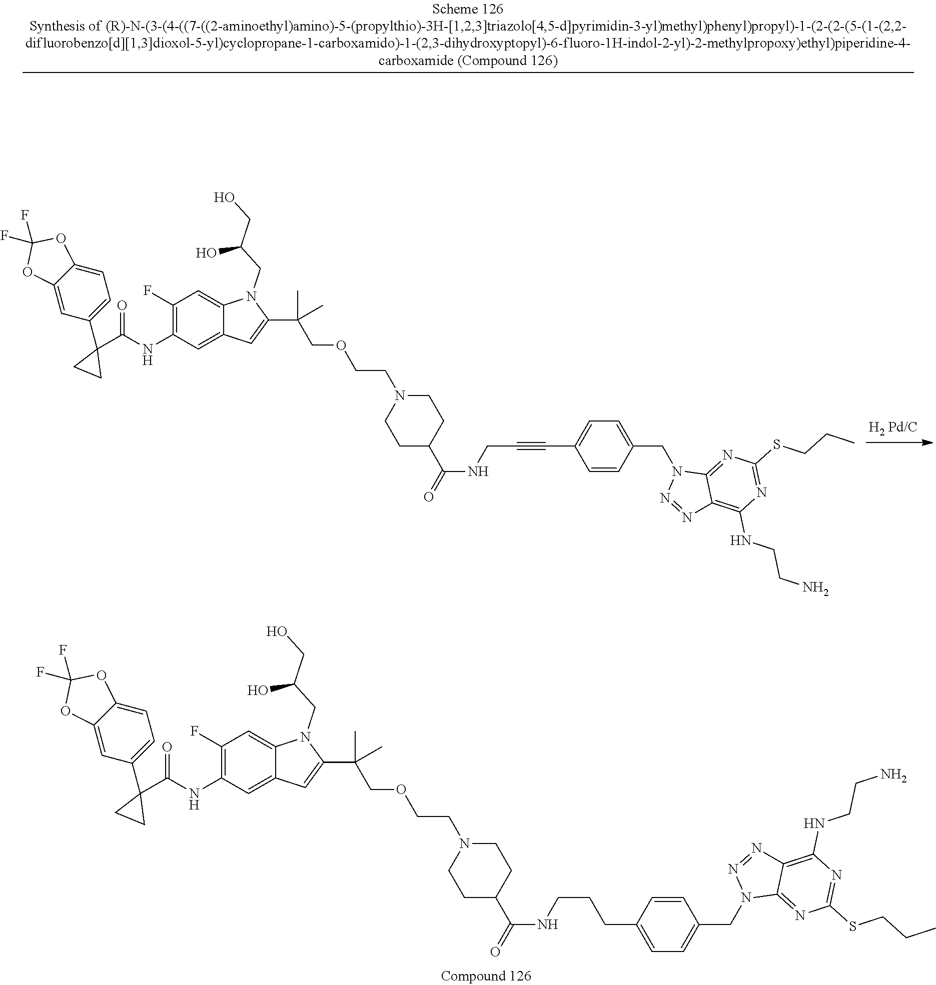

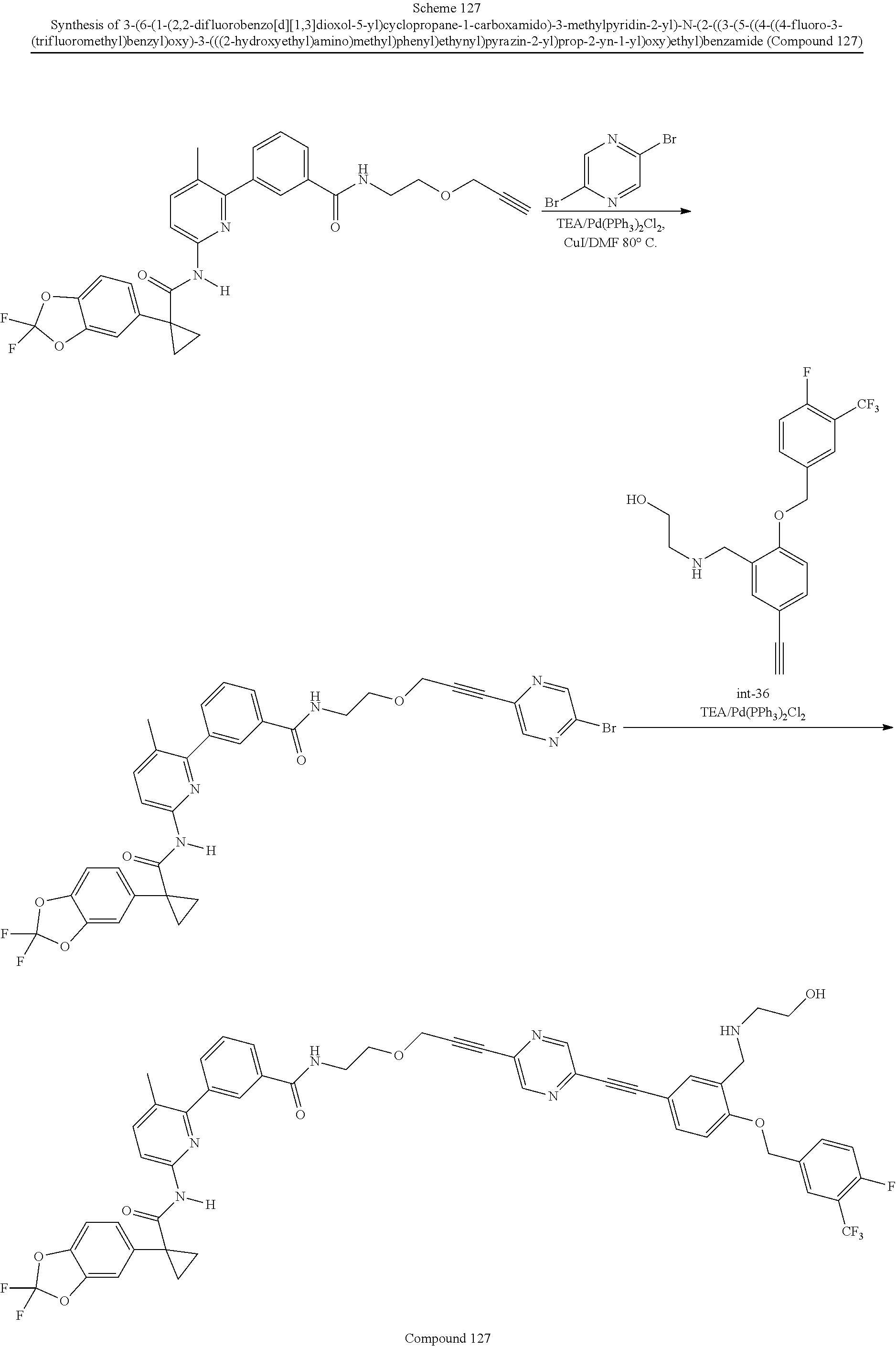

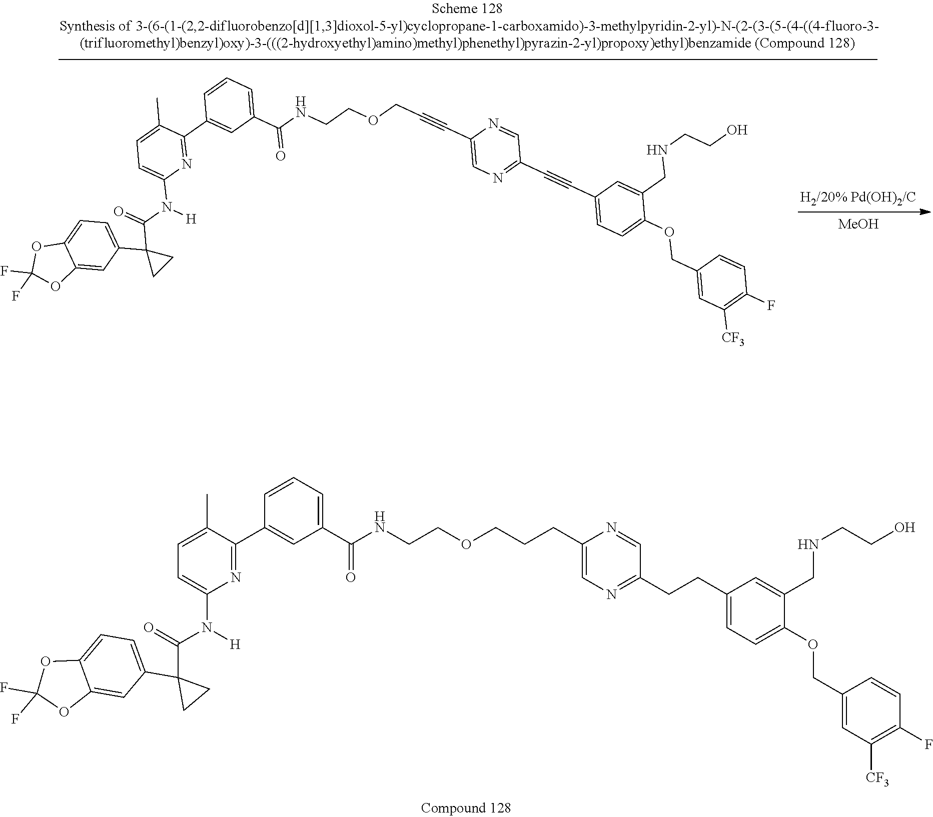 [SMILES: "MeOH" ; Cc1ccc(NC(=O)C2(c3ccc4c(c3)OC(F)(F)O4)CC2)nc1-c1cccc(C(=O)NCCOCC#Cc2cnc(C#Cc3ccc(OCc4ccc(F)c(C(F)(F)F)c4)c(CNCCO)c3)cn2)c1 ; Cc1ccc(NC(=O)C2(c3ccc4c(c3)OC(F)(F)O4)CC2)nc1-c1cccc(C(=O)NCCOCCCc2cnc(CCc3ccc(OCc4ccc(F)c(C(F)(F)F)c4)c(CNCCO)c3)cn2)c1]

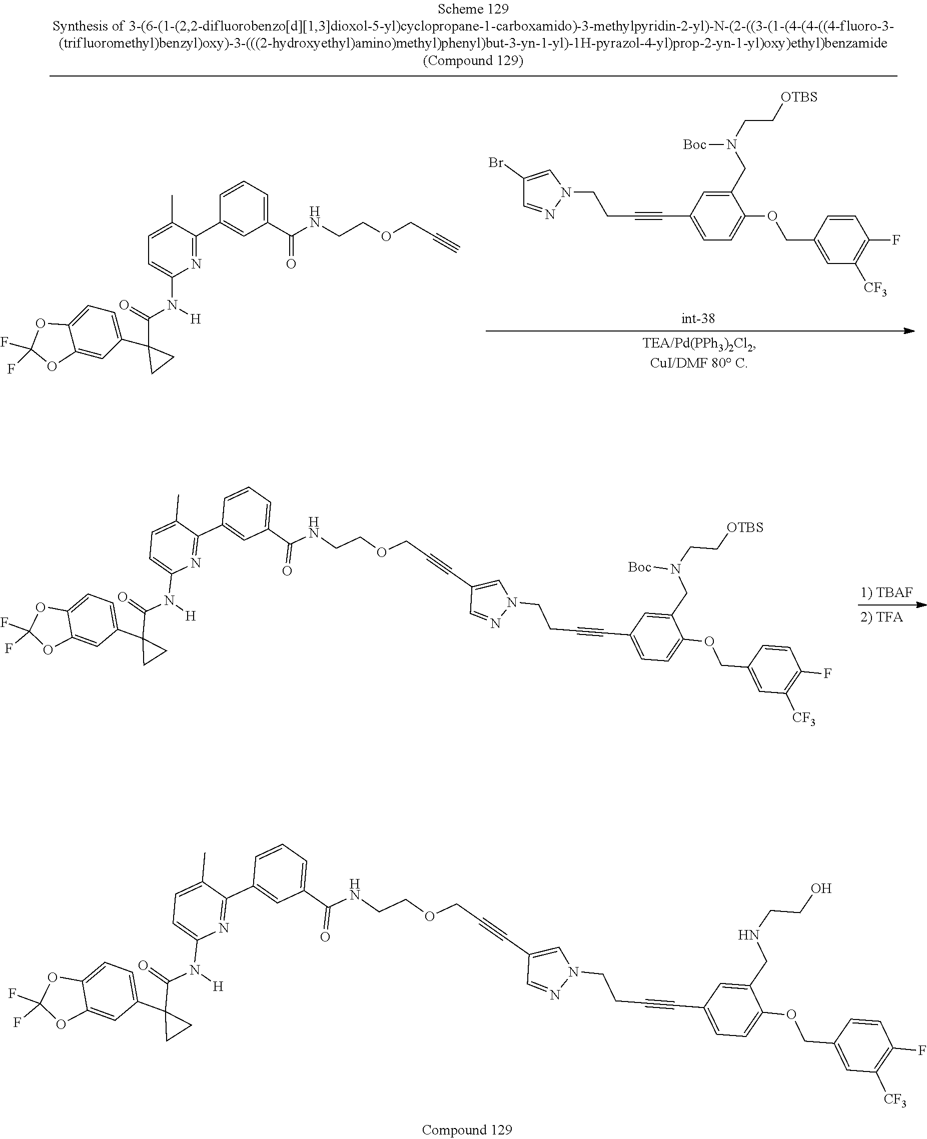 [SMILES: C#CCOCCNC(=O)c1cccc(-c2nc(NC(=O)C3(c4ccc5c(c4)OC(F)(F)O5)CC3)ccc2C)c1 ; CC(C)(C)OC(=O)N(CCO[Si](C)(C)C(C)(C)C)Cc1cc(C#CCCn2cc(Br)cn2)ccc1OCc1ccc(F)c(C(F)(F)F)c1 ; Cc1ccc(NC(=O)C2(c3ccc4c(c3)OC(F)(F)O4)CC2)nc1-c1cccc(C(=O)NCCOCC#Cc2cnn(CCC#Cc3ccc(OCc4ccc(F)c(C(F)(F)F)c4)c(CN(CCO[Si](C)(C)C(C)(C)C)C(=O)OC(C)(C)C)c3)c2)c1 ; Cc1ccc(NC(=O)C2(c3ccc4c(c3)OC(F)(F)O4)CC2)nc1-c1cccc(C(=O)NCCOCC#Cc2cnn(CCC#Cc3ccc(OCc4ccc(F)c(C(F)(F)F)c4)c(CNCCO)c3)c2)c1]

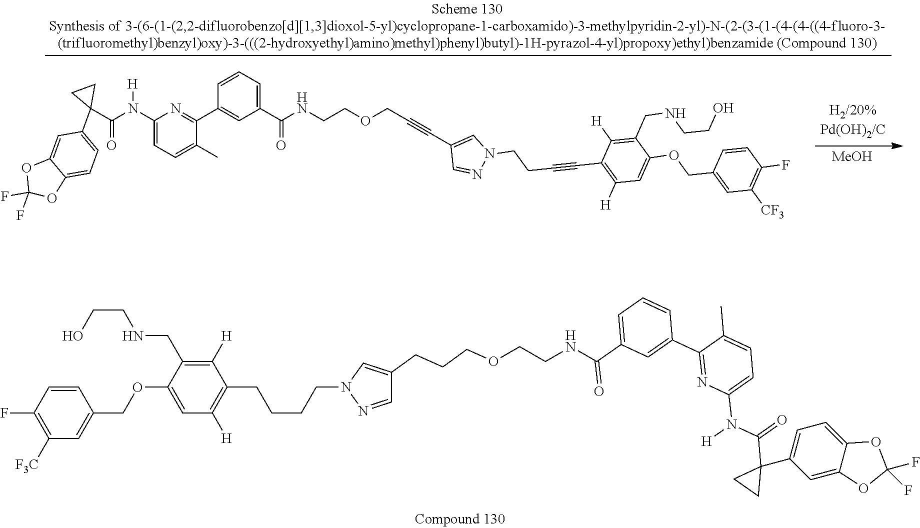 [SMILES: "MeOH" ; Cc1ccc(NC(=O)C2(c3ccc4c(c3)OC(F)(F)O4)CC2)nc1-c1cccc(C(=O)NCCOCC#Cc2cnn(CCC#Cc3ccc(OCc4ccc(F)c(C(F)(F)F)c4)c(CNCCO)c3)c2)c1 ; Cc1ccc(NC(=O)C2(c3ccc4c(c3)OC(F)(F)O4)CC2)nc1-c1cccc(C(=O)NCCOCCCc2cnn(CCCCc3ccc(OCc4ccc(F)c(C(F)(F)F)c4)c(CNCCO)c3)c2)c1]

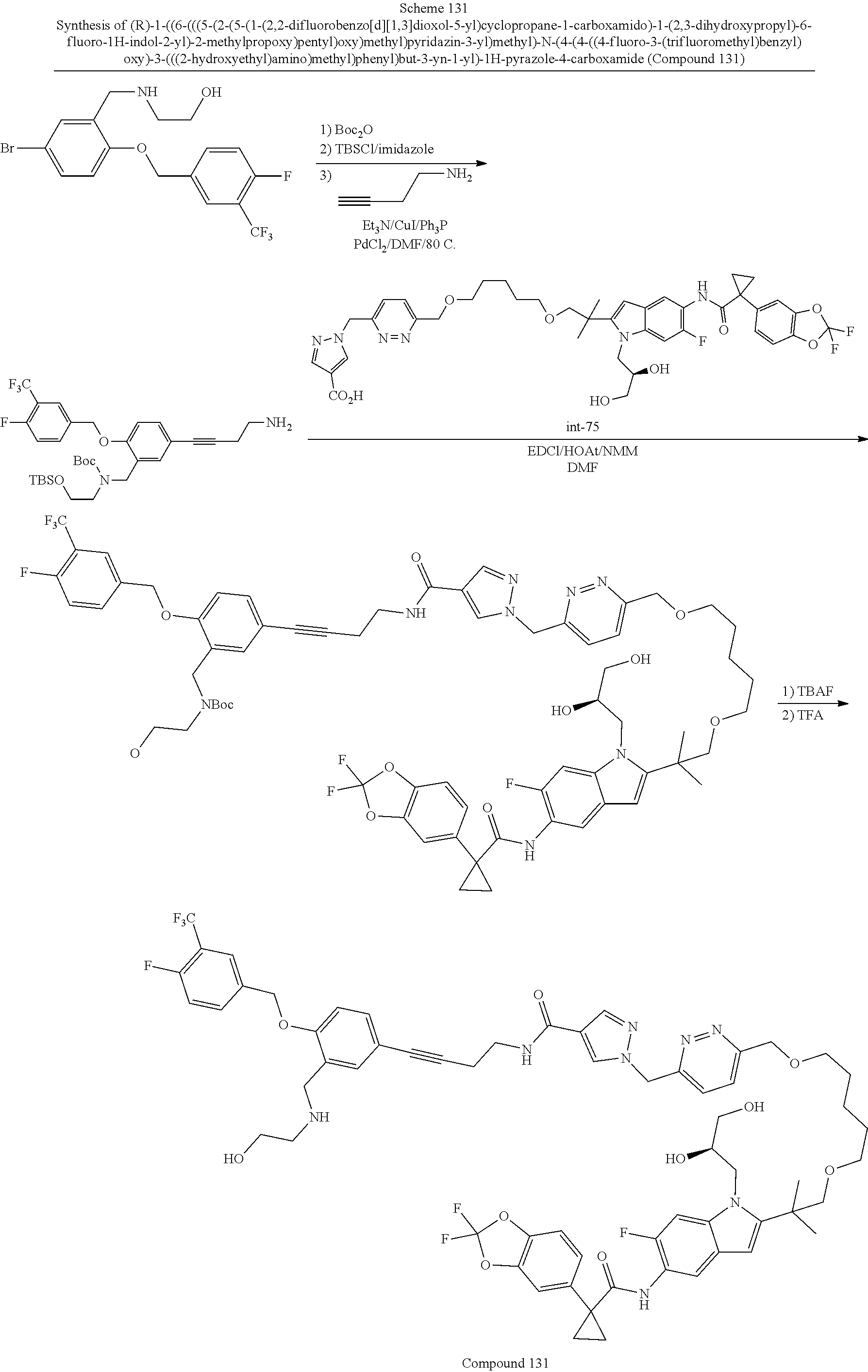

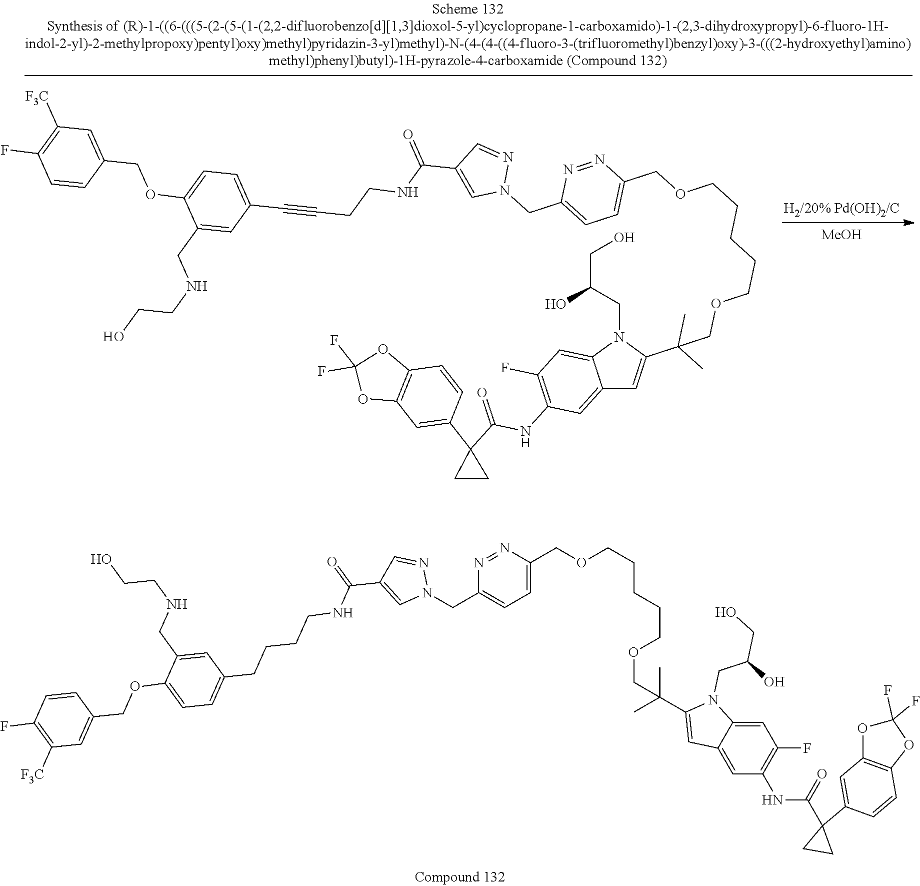 [SMILES: "MeOH" ; CC(C)(COCCCCCOCc1ccc(Cn2cc(C(=O)NCCC#Cc3ccc(OCc4ccc(F)c(C(F)(F)F)c4)c(CNCCO)c3)cn2)nn1)c1cc2cc(NC(=O)C3(c4ccc5c(c4)OC(F)(F)O5)CC3)c(F)cc2n1C[C@@H](O)CO ; CC(C)(COCCCCCOCc1ccc(Cn2cc(C(=O)NCCCCc3ccc(OCc4ccc(F)c(C(F)(F)F)c4)c(CNCCO)c3)cn2)nn1)c1cc2cc(NC(=O)C3(c4ccc5c(c4)OC(F)(F)O5)CC3)c(F)cc2n1C[C@H](O)CO]

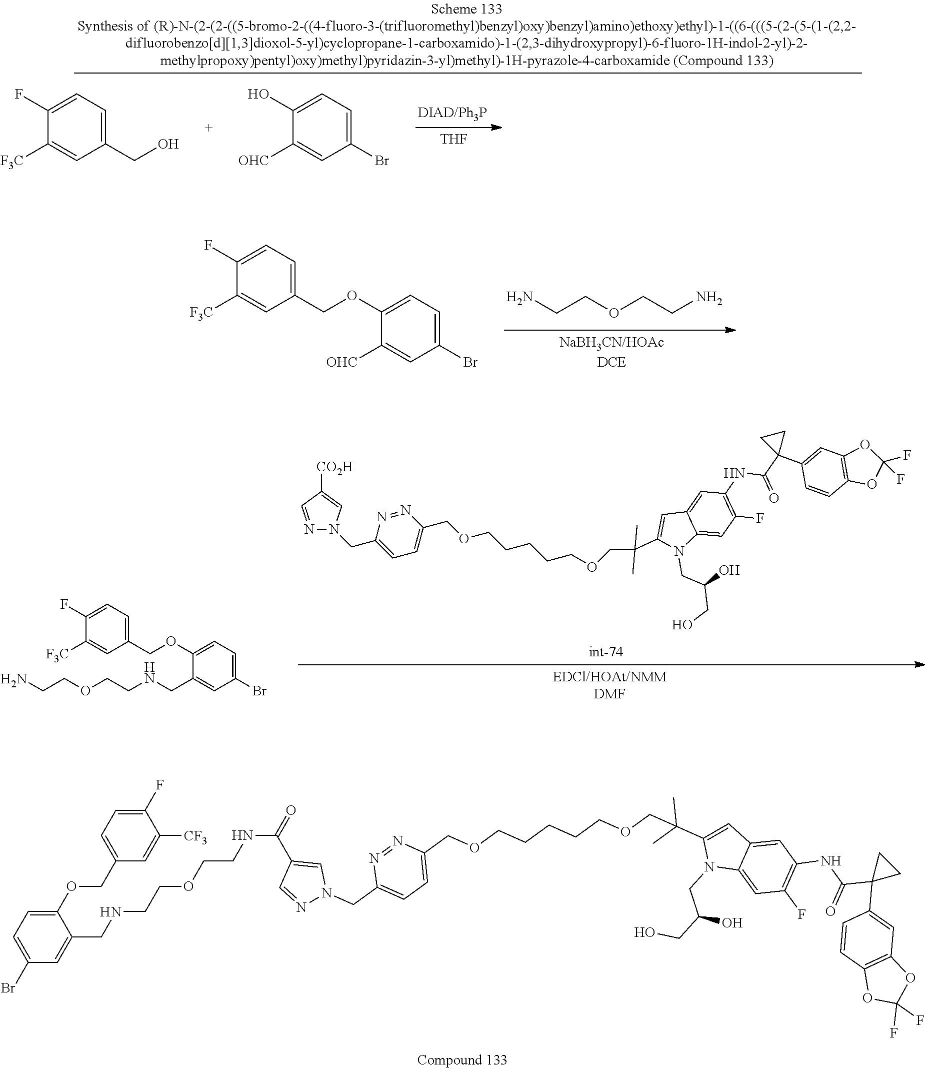

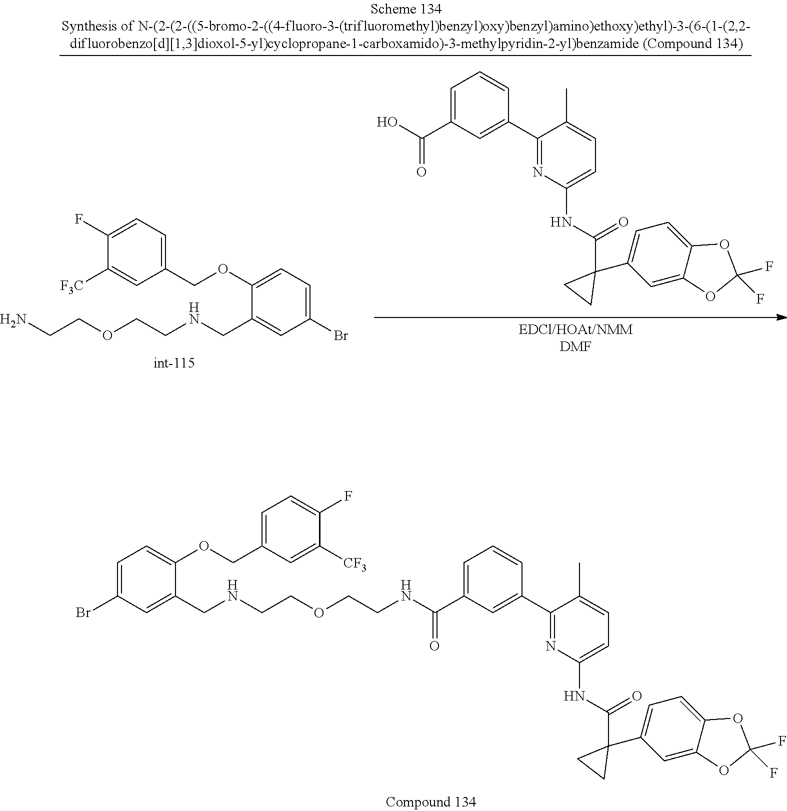 [SMILES: NCCOCCNCc1cc(Br)ccc1OCc1ccc(F)c(C(F)(F)F)c1 ; Cc1ccc(NC(=O)C2(c3ccc4c(c3)OC(F)(F)O4)CC2)nc1-c1cccc(C(=O)O)c1 ; Cc1ccc(NC(=O)C2(c3ccc4c(c3)OC(F)(F)O4)CC2)nc1-c1cccc(C(=O)NCCOCCNCc2cc(Br)ccc2OCc2ccc(F)c(C(F)(F)F)c2)c1]

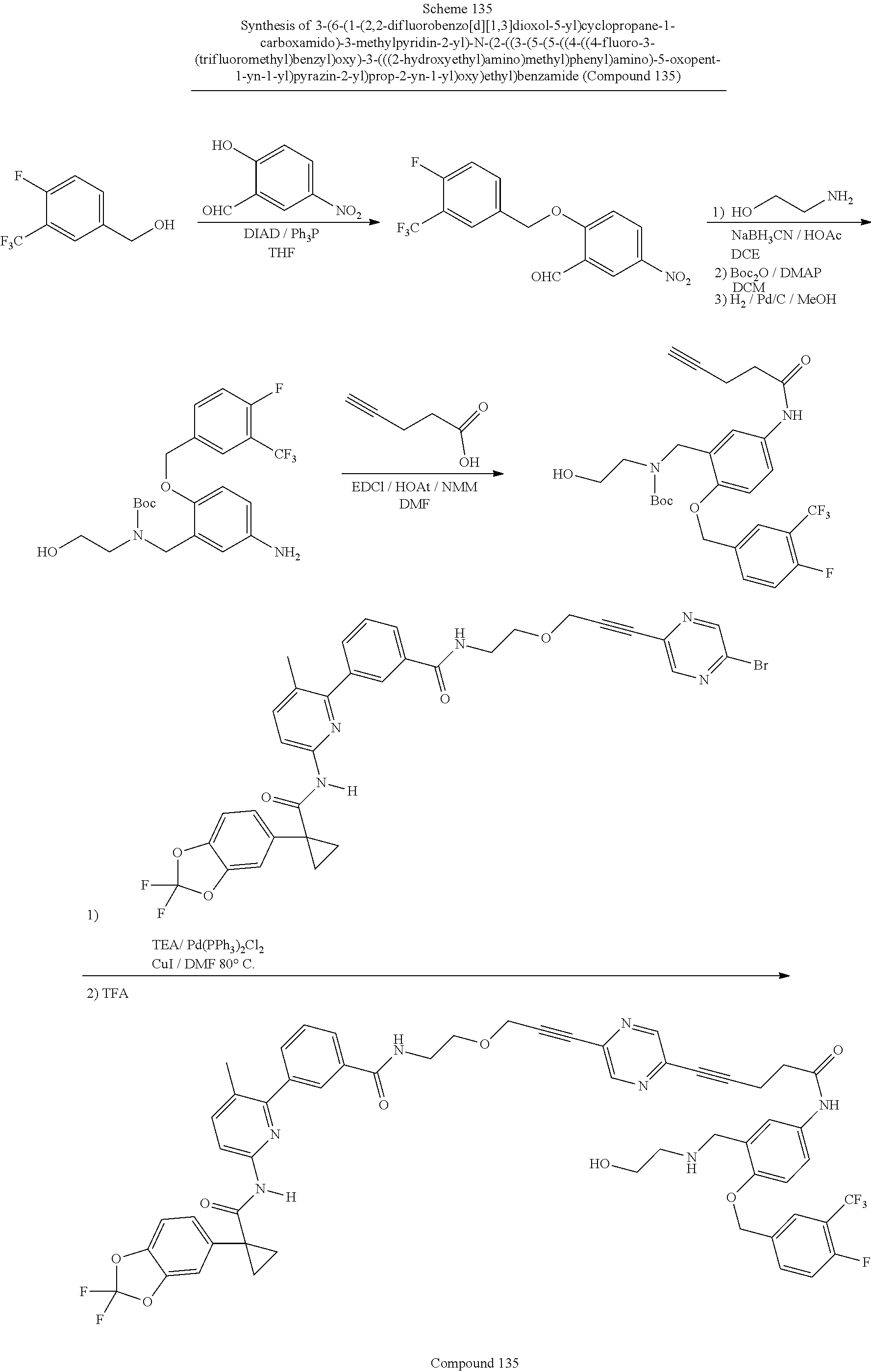

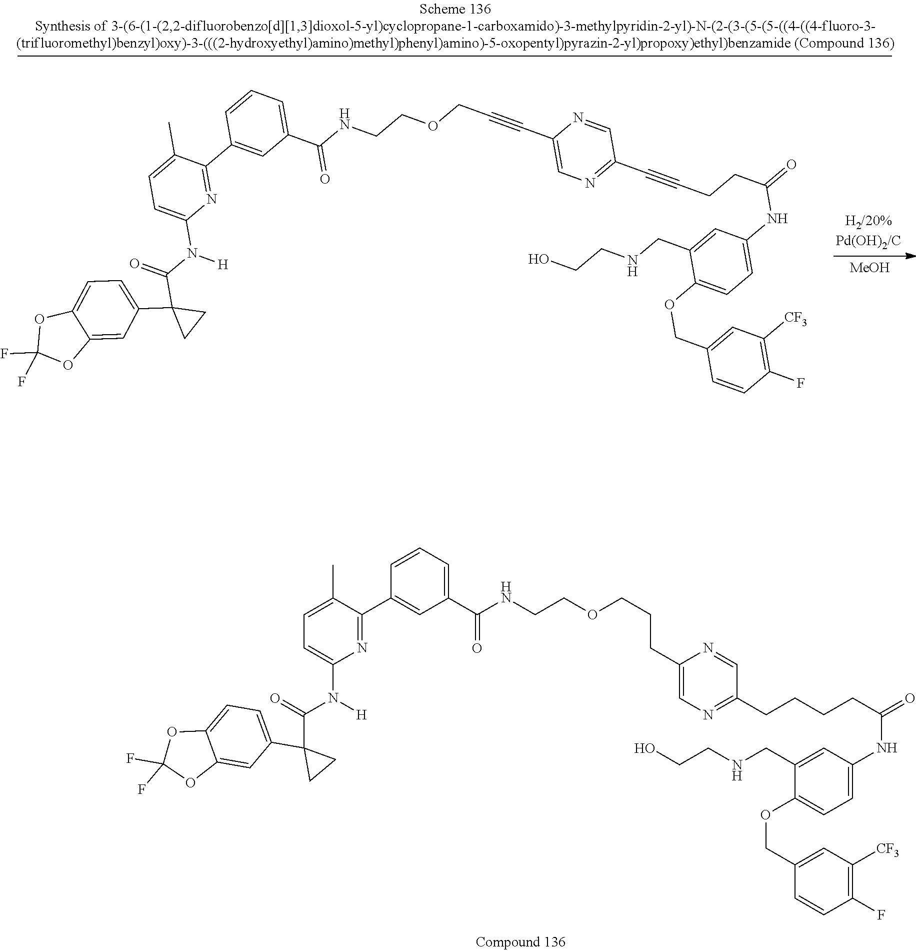 [SMILES: "MeOH" ; Cc1ccc(NC(=O)C2(c3ccc4c(c3)OC(F)(F)O4)CC2)nc1-c1cccc(C(=O)NCCOCC#Cc2cnc(C#CCCC(=O)Nc3ccc(OCc4ccc(F)c(C(F)(F)F)c4)c(CNCCO)c3)cn2)c1 ; Cc1ccc(NC(=O)C2(c3ccc4c(c3)OC(F)(F)O4)CC2)nc1-c1cccc(C(=O)NCCOCCCc2cnc(CCCCC(=O)Nc3ccc(OCc4ccc(F)c(C(F)(F)F)c4)c(CNCCO)c3)cn2)c1]

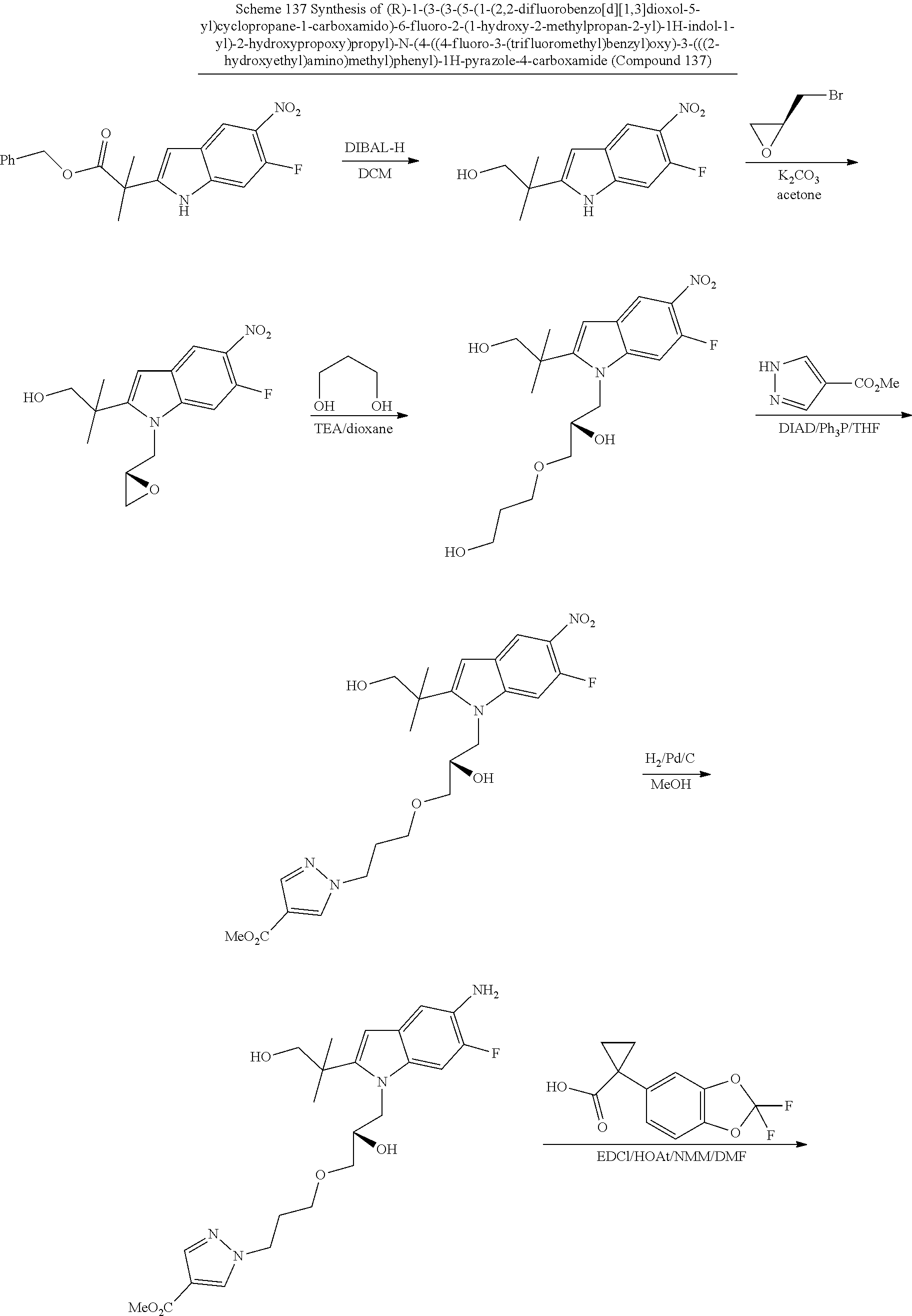

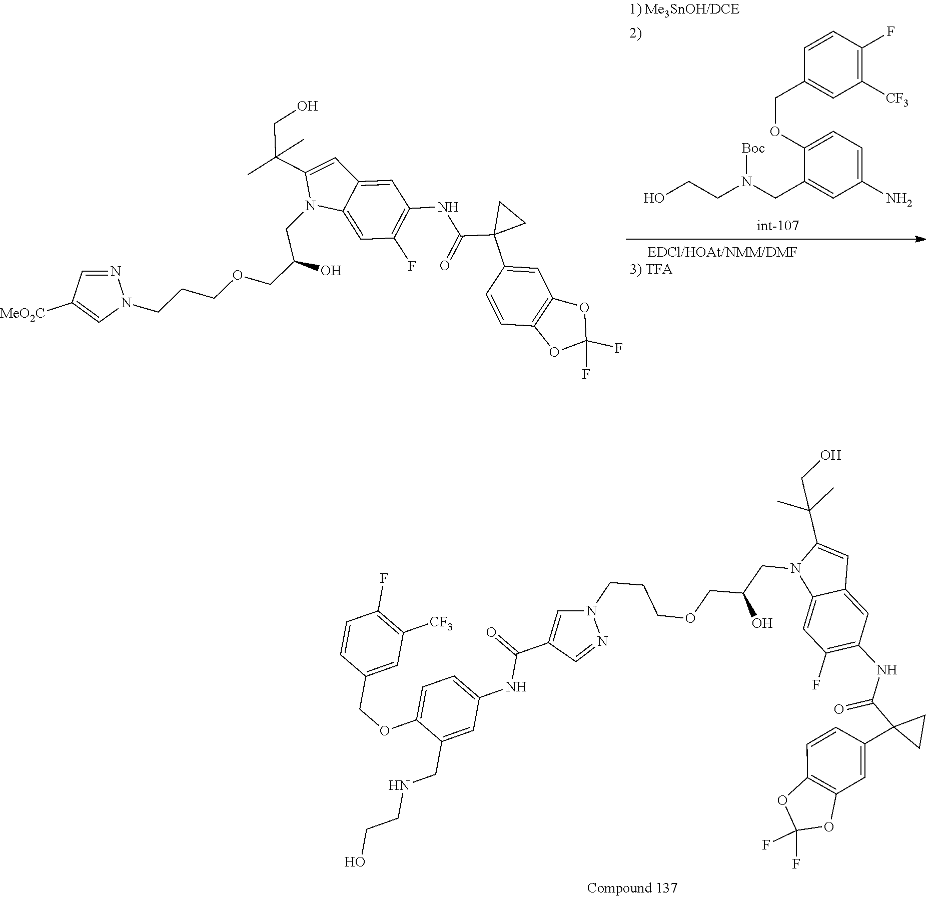 [SMILES: COC(=O)c1cnn(CCCOC[C@H](O)Cn2c(C(C)(C)CO)cc3cc(NC(=O)C4(c5ccc6c(c5)OC(F)(F)O6)CC4)c(F)cc32)c1 ; CC(C)(C)OC(=O)N(CCO)Cc1cc(N)ccc1OCc1ccc(F)c(C(F)(F)F)c1 ; CC(C)(CO)c1cc2cc(NC(=O)C3(c4ccc5c(c4)OC(F)(F)O5)CC3)c(F)cc2n1C[C@@H](O)COCCCn1cc(C(=O)Nc2ccc(OCc3ccc(F)c(C(F)(F)F)c3)c(CNCCO)c2)cn1]

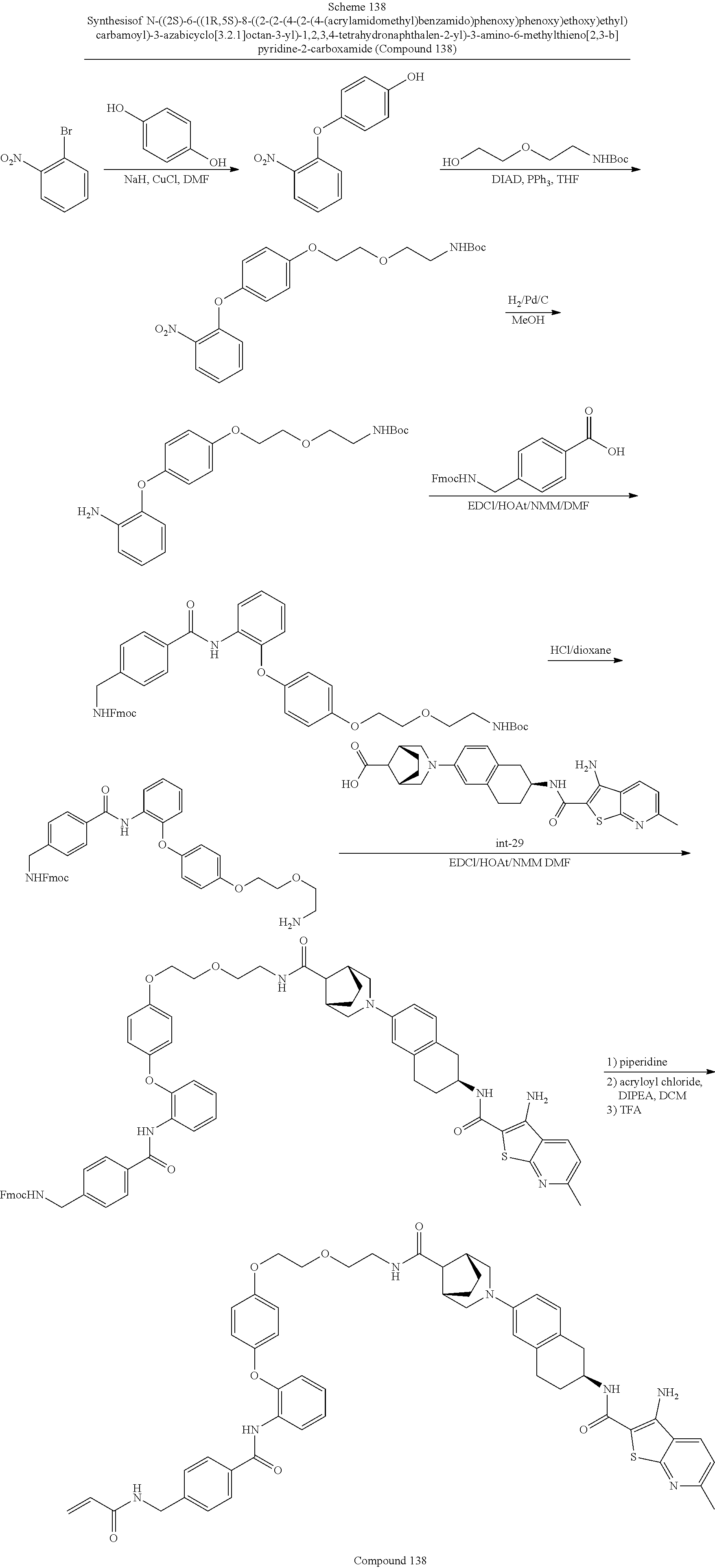

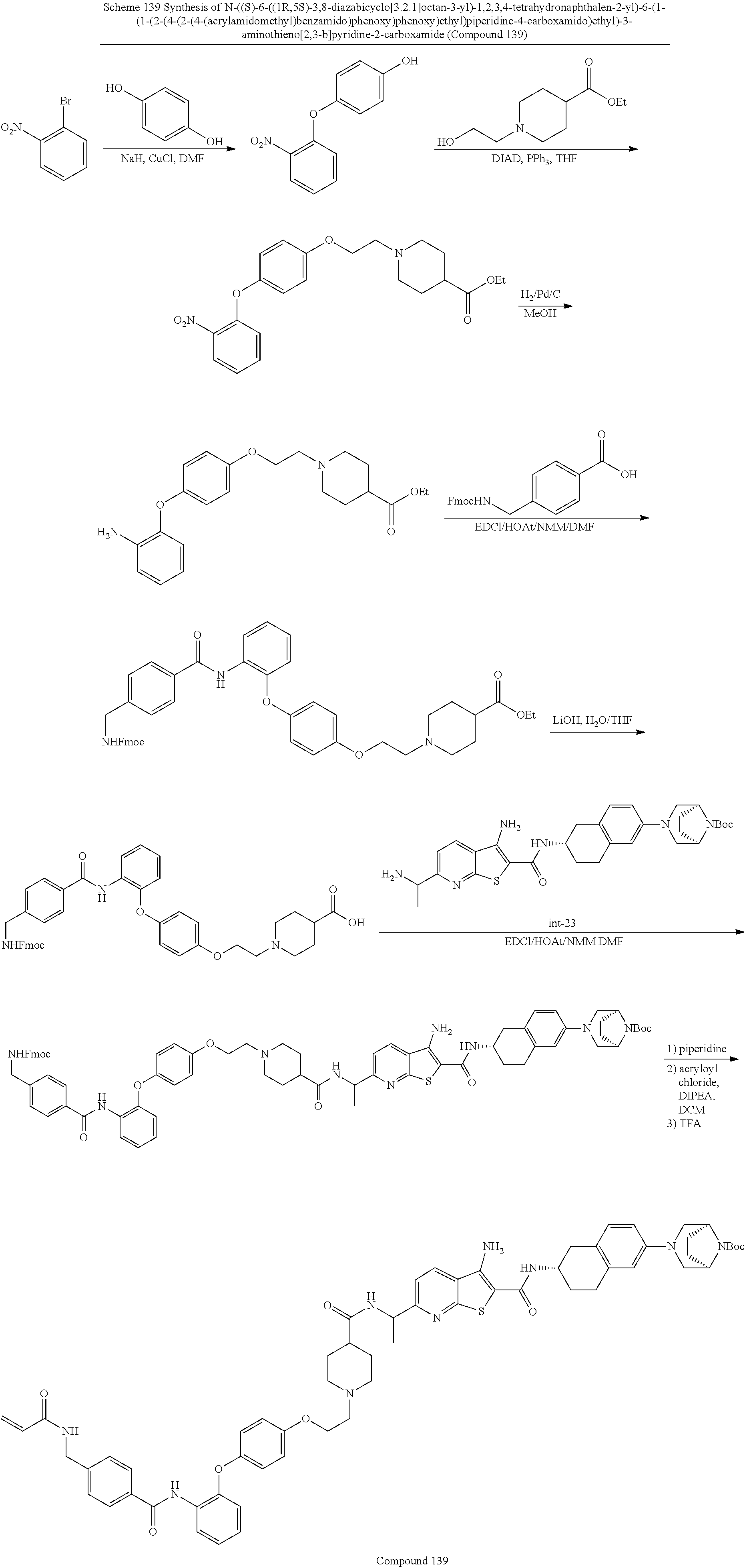

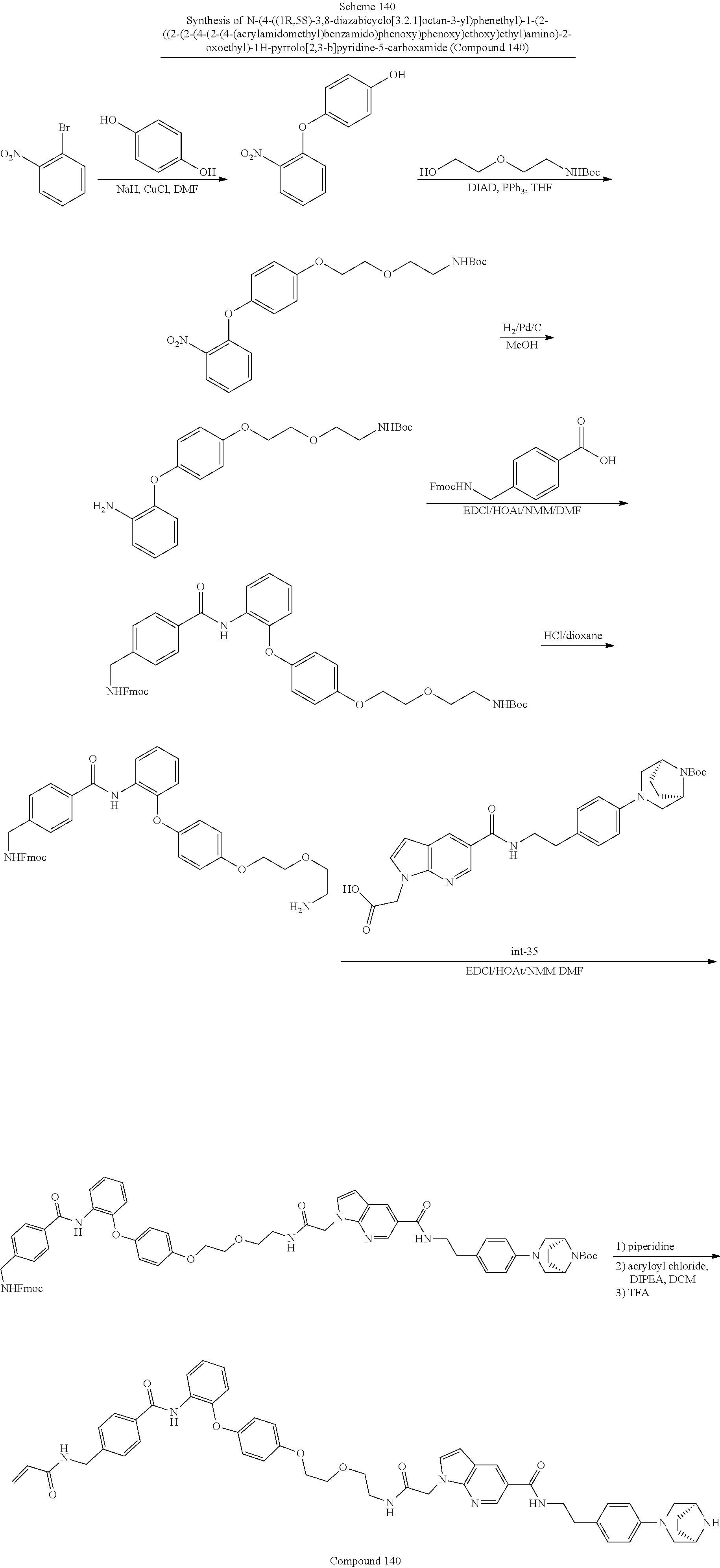

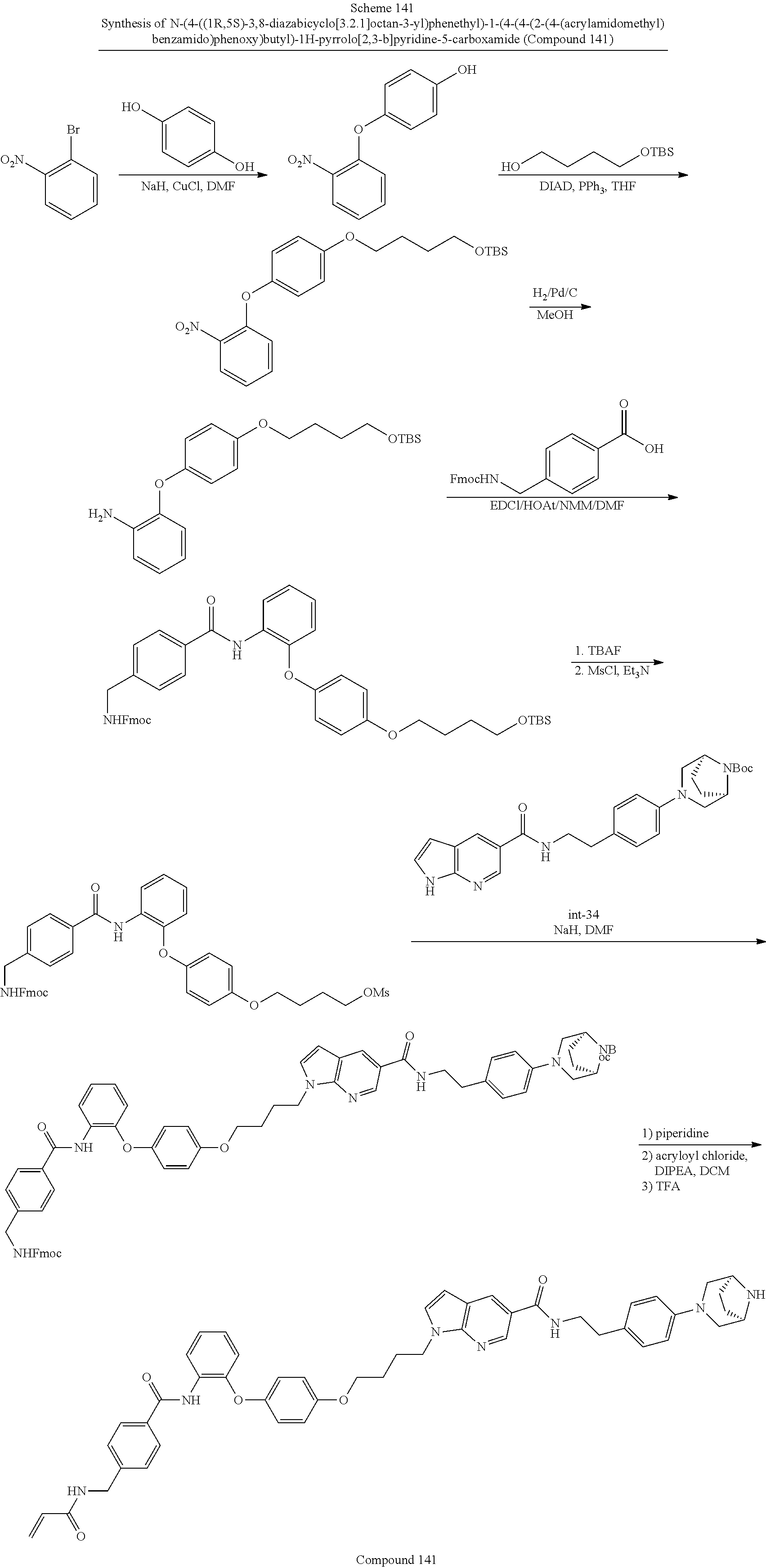

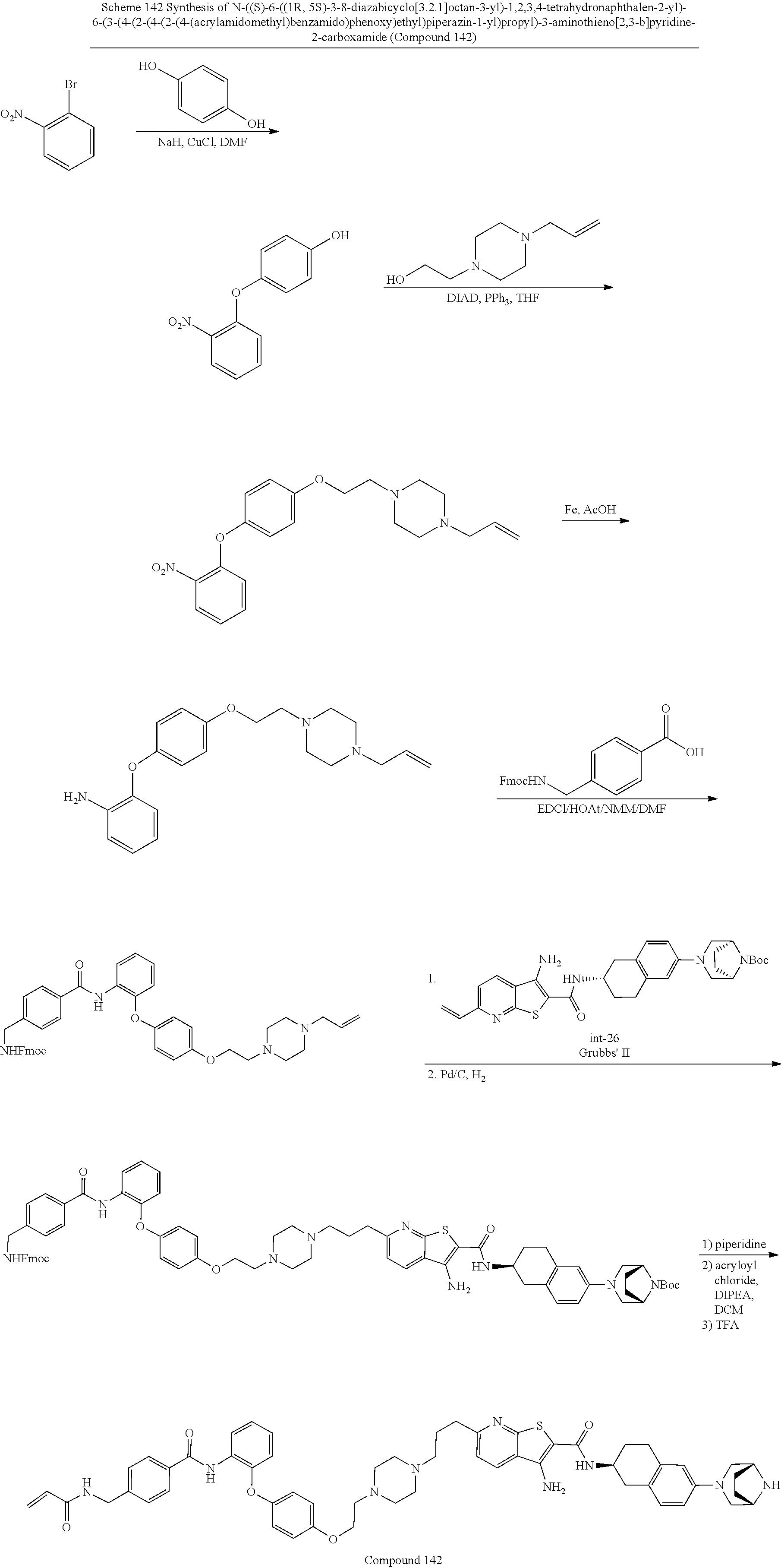

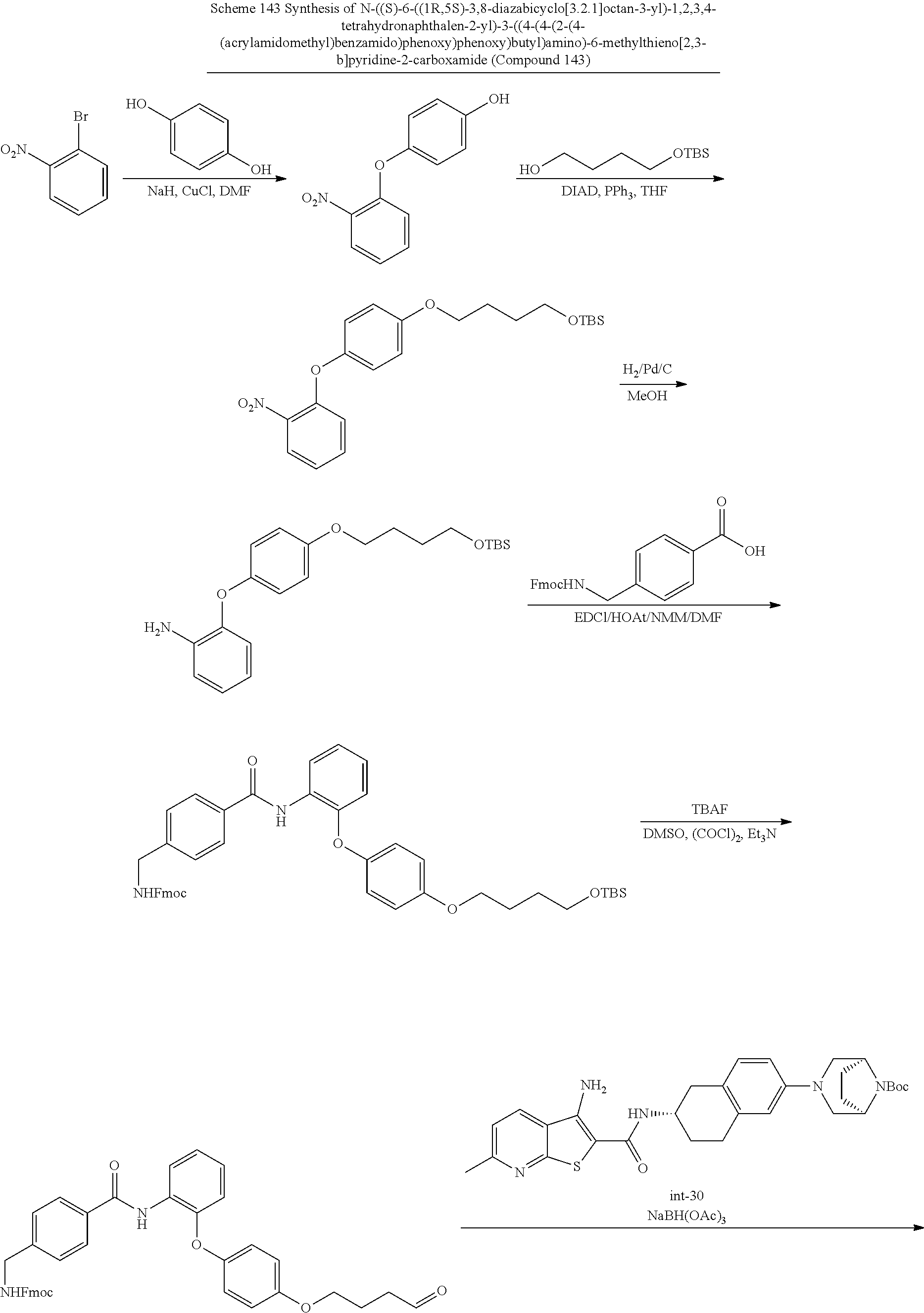

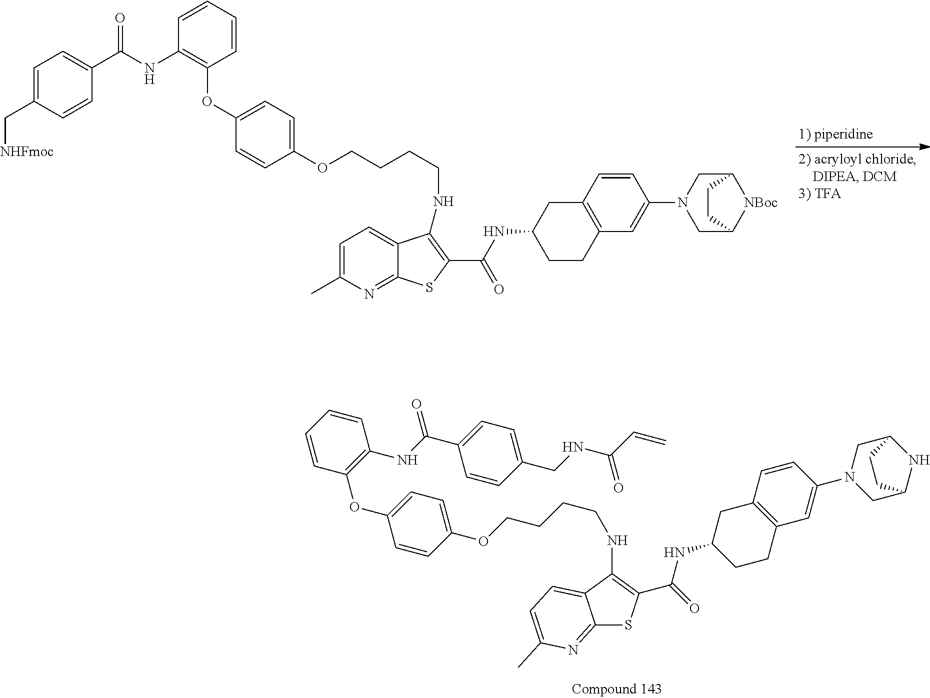

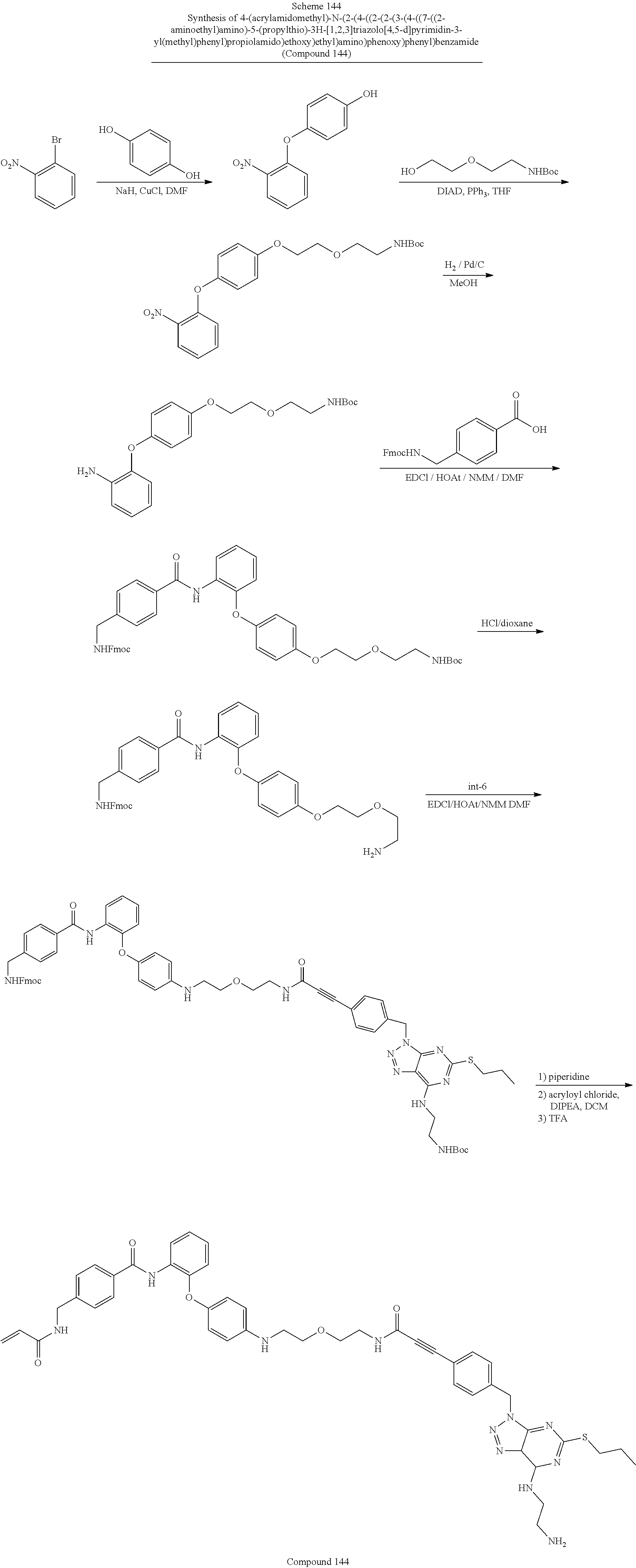

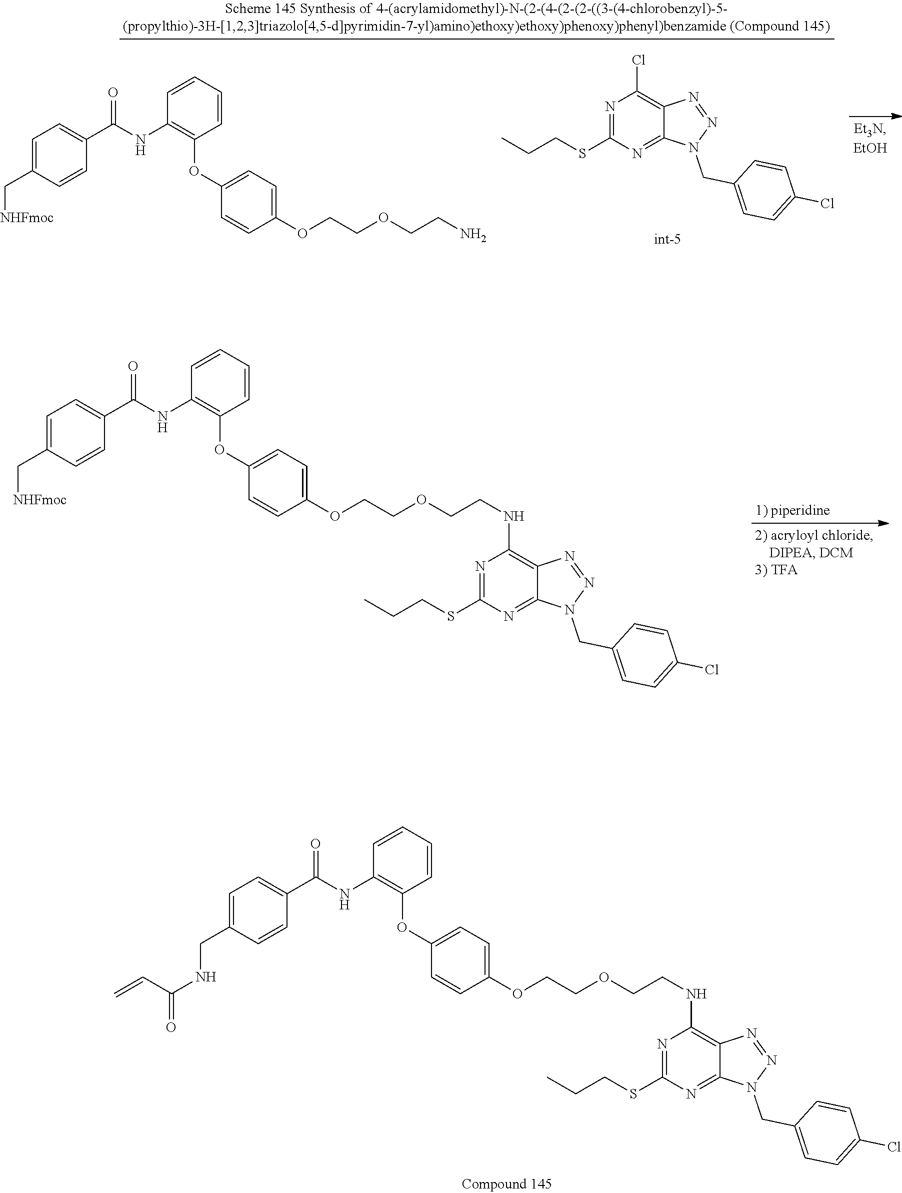

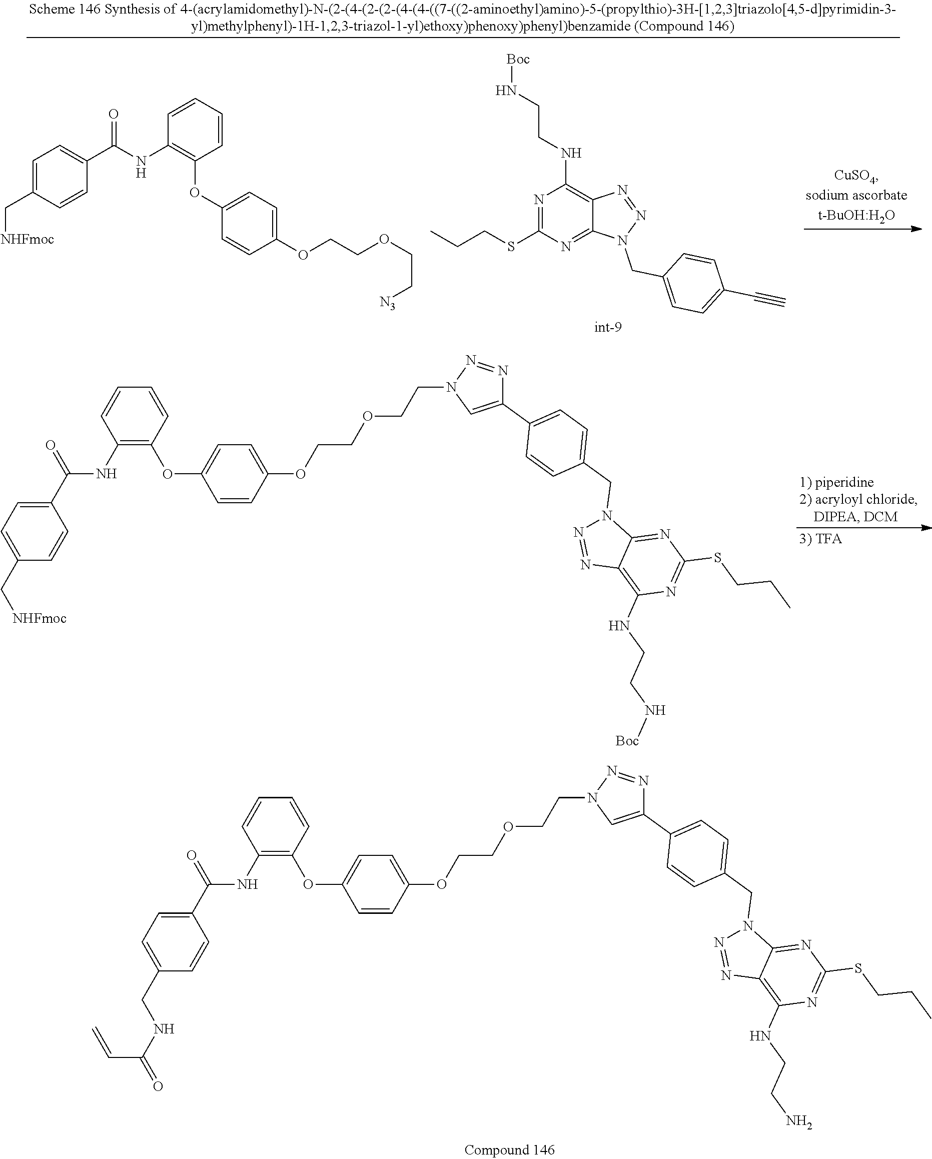

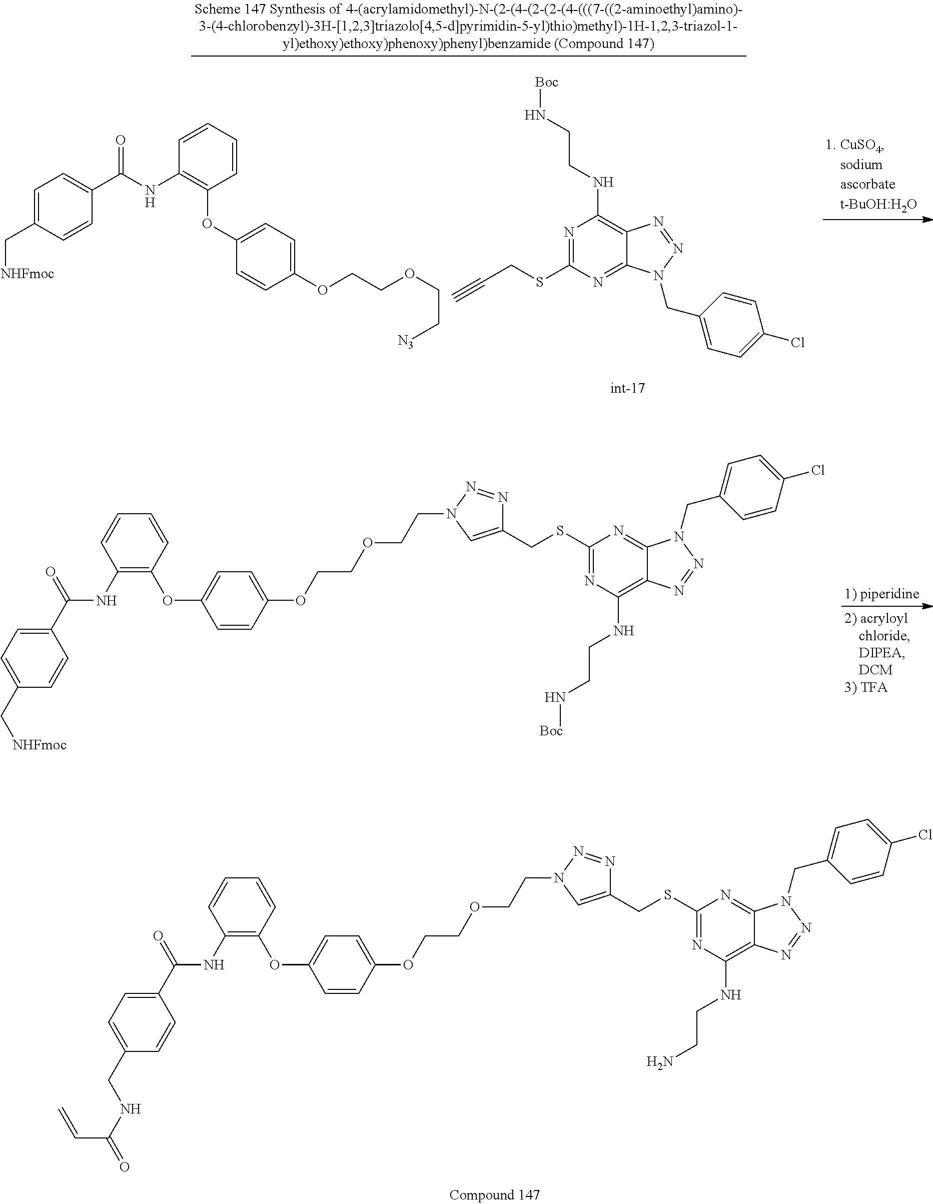

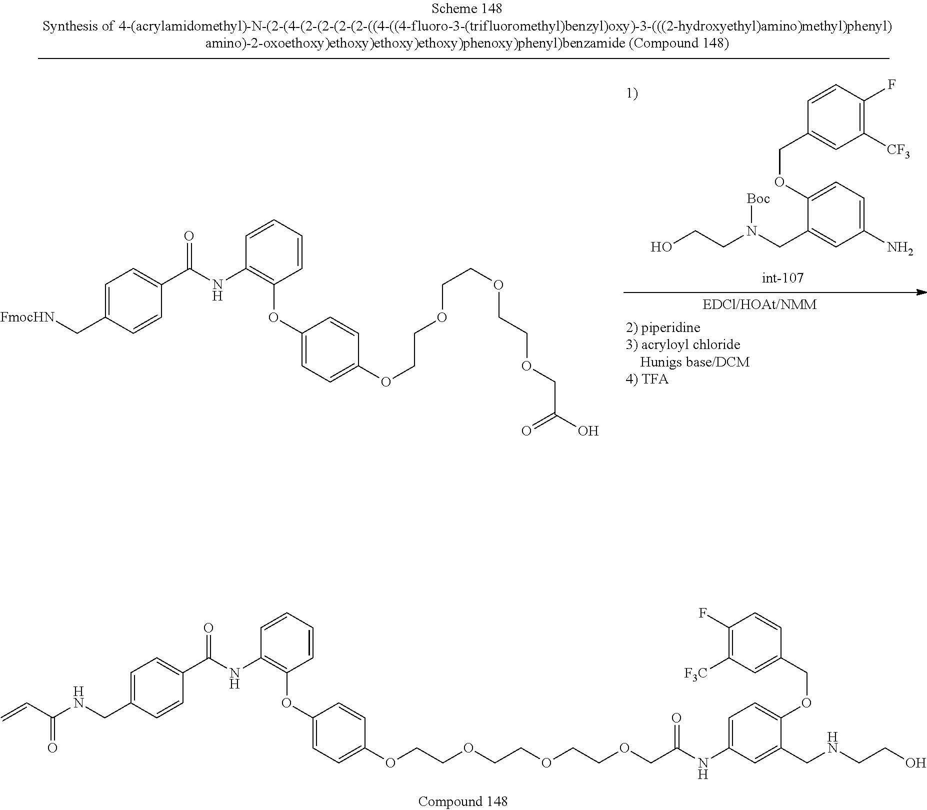

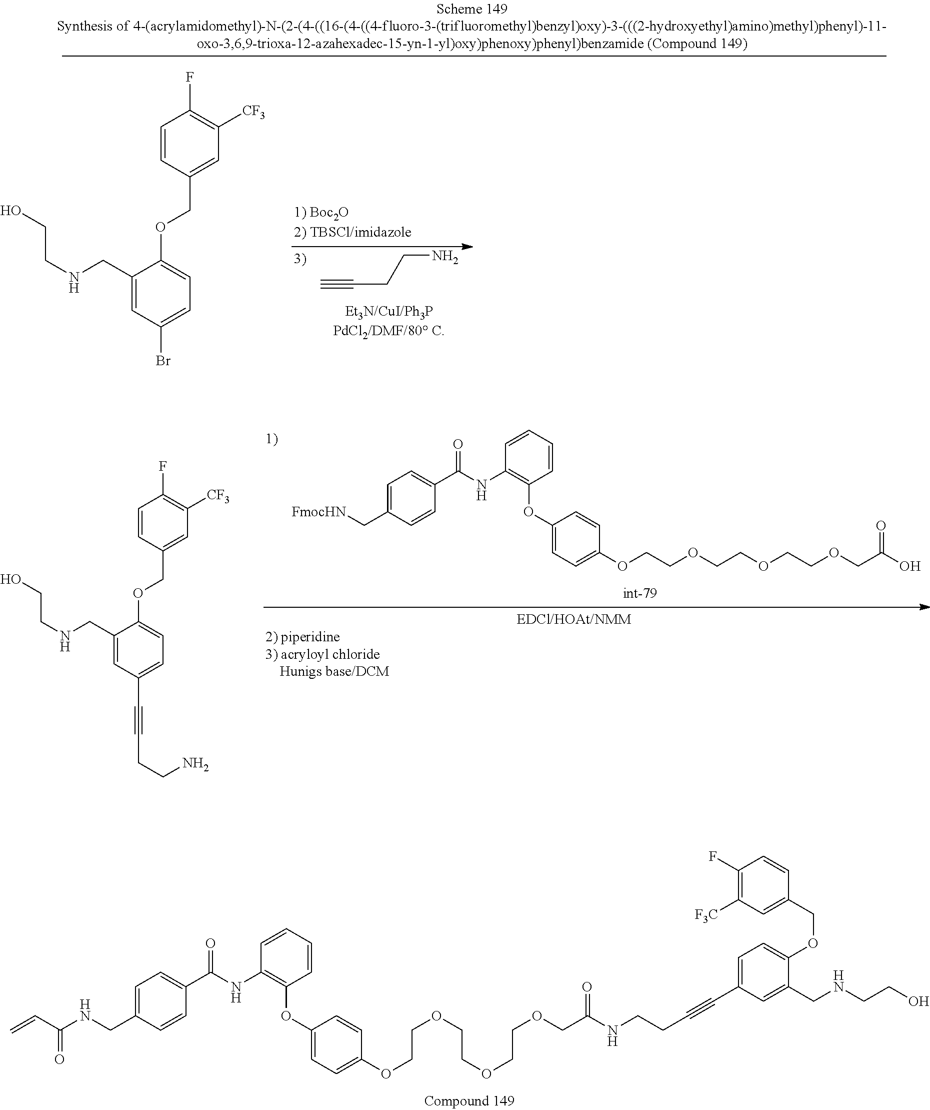

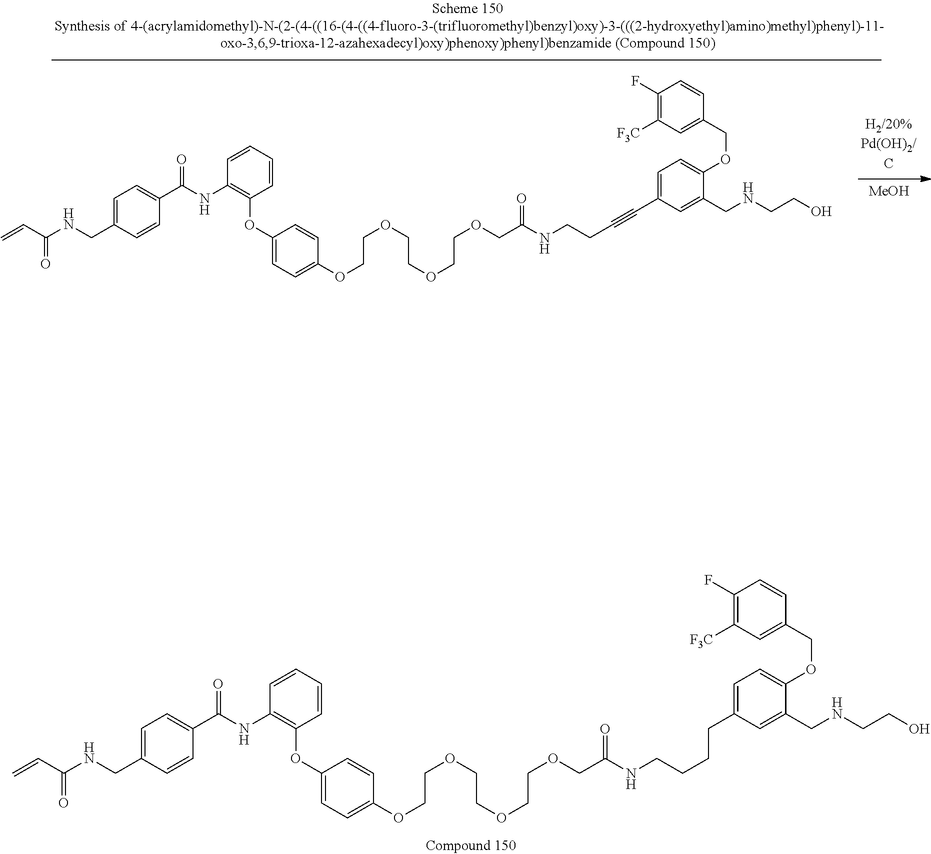 [SMILES: "MeOH" ; C=CC(=O)NCc1ccc(C(=O)Nc2ccccc2Oc2ccc(OCCOCCOCCOCC(=O)NCCC#Cc3ccc(OCc4ccc(F)c(C(F)(F)F)c4)c(CNCCO)c3)cc2)cc1 ; C=CC(=O)NCc1ccc(C(=O)Nc2ccccc2Oc2ccc(OCCOCCOCCOCC(=O)NCCCCc3ccc(OCc4ccc(F)c(C(F)(F)F)c4)c(CNCCO)c3)cc2)cc1]

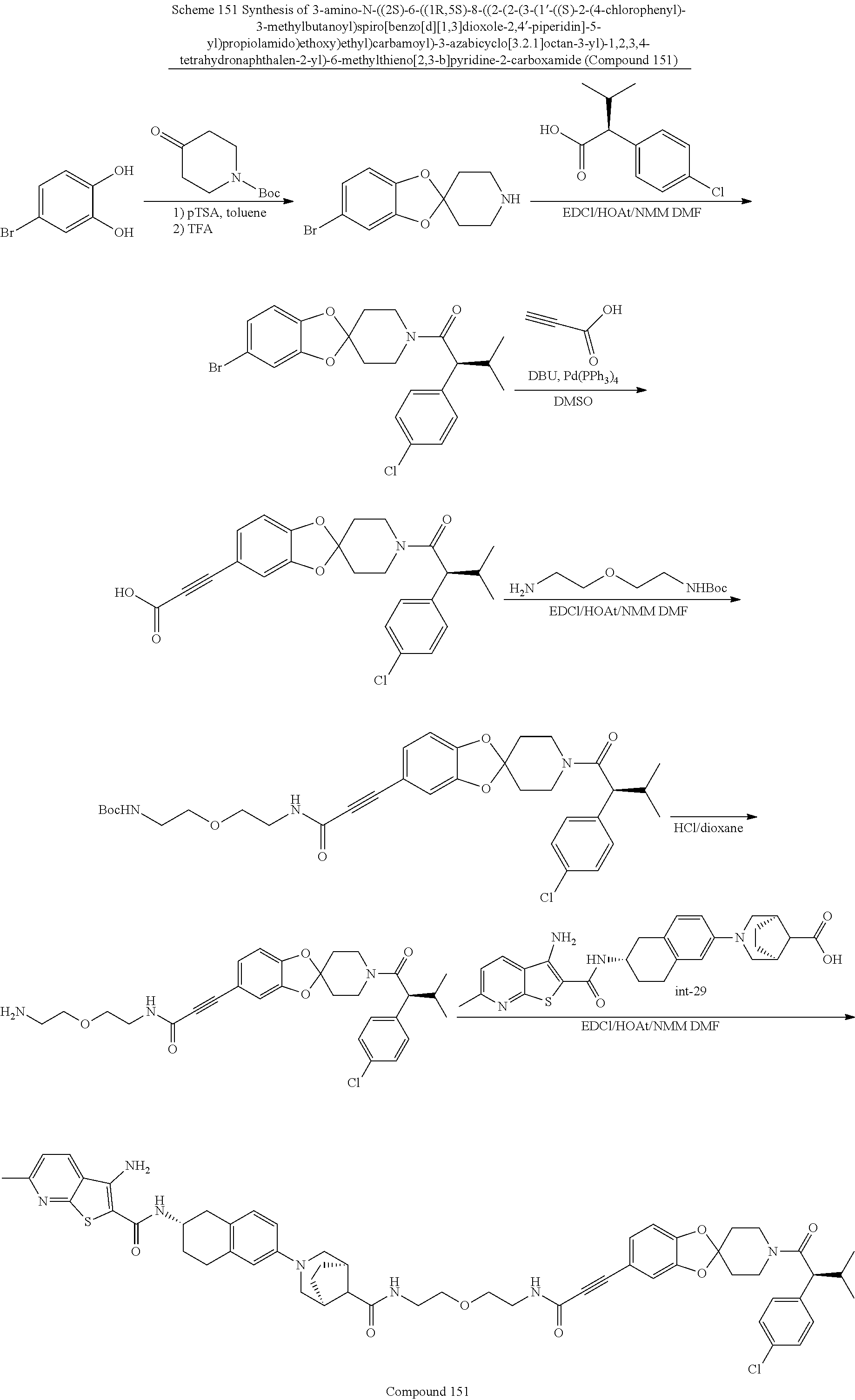

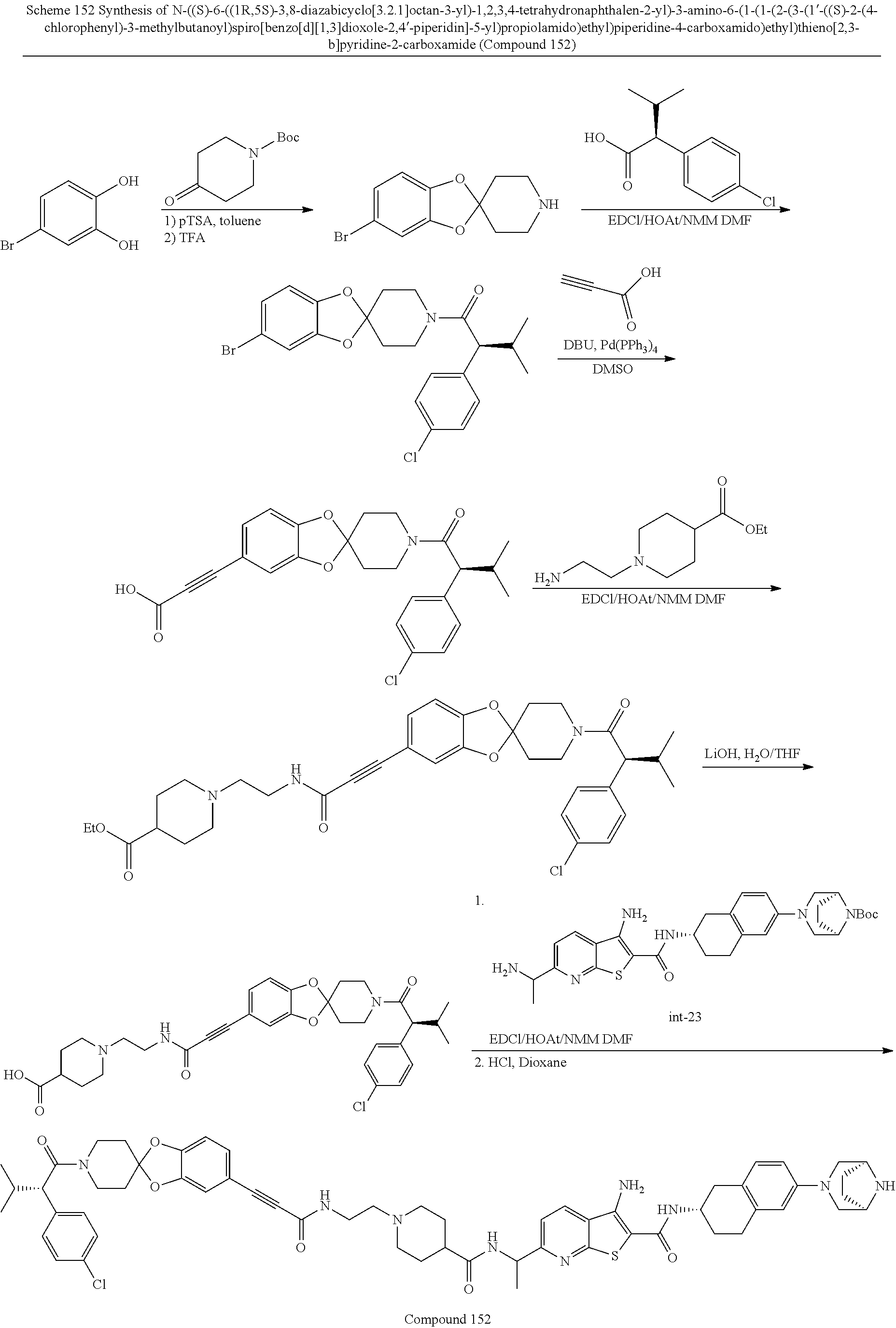

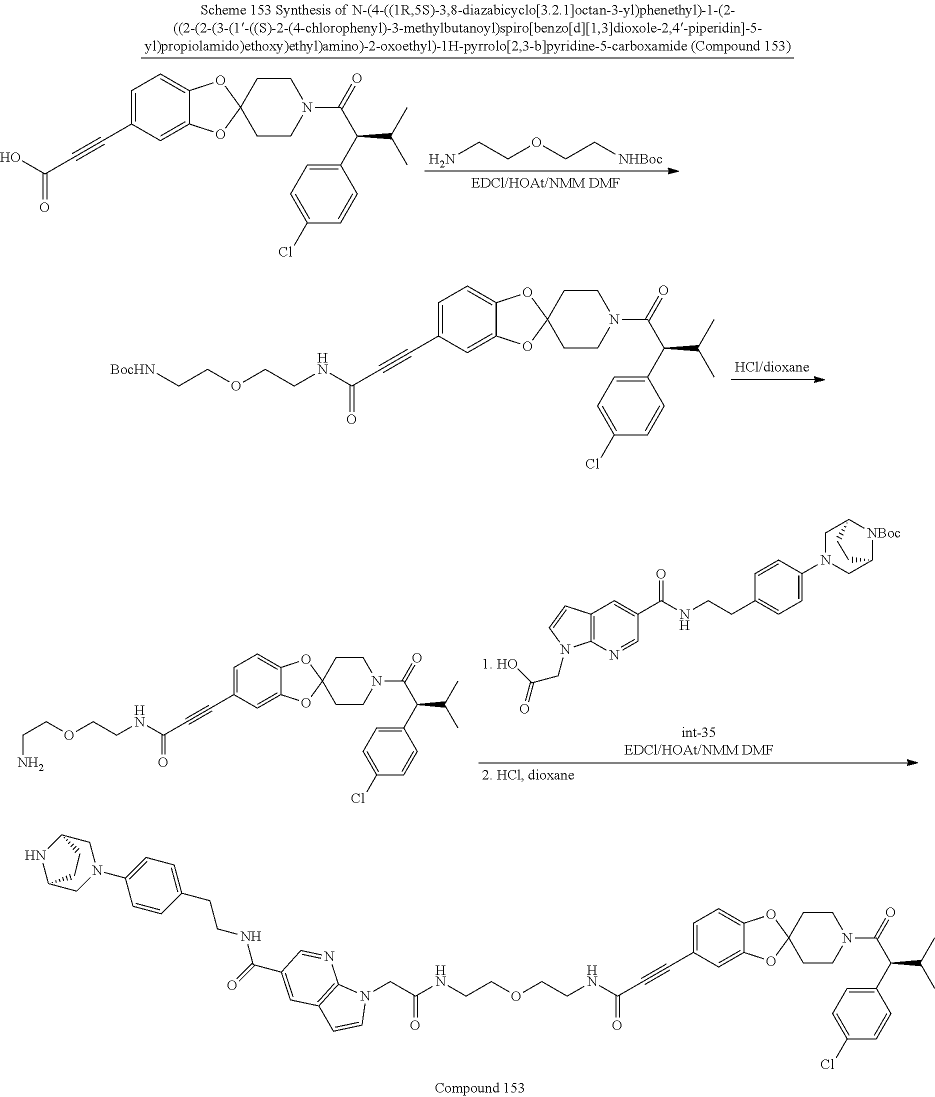

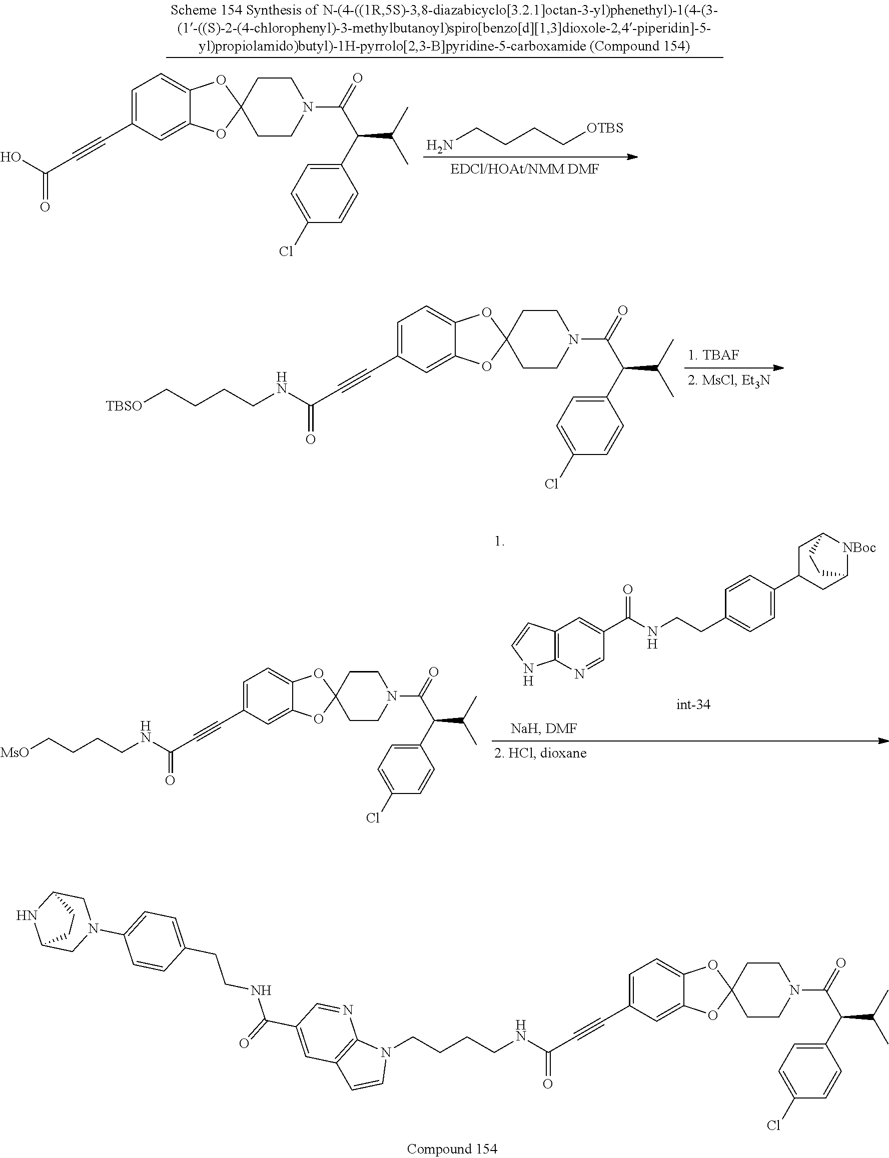 [SMILES: CC(C)[C@H](C(=O)N1CCC2(CC1)Oc1ccc(C#CC(=O)O)cc1O2)c1ccc(Cl)cc1 ; CC(C)(C)[Si](C)(C)OCCCCN ; CC(C)[C@H](C(=O)N1CCC2(CC1)Oc1ccc(C#CC(=O)NCCCCO[Si](C)(C)C(C)(C)C)cc1O2)c1ccc(Cl)cc1 ; CC(C)[C@H](C(=O)N1CCC2(CC1)Oc1ccc(C#CC(=O)NCCCCOS(C)(=O)=O)cc1O2)c1ccc(Cl)cc1 ; CC(C)(C)OC(=O)N1[C@@H]2CC[C@H]1CC(c1ccc(CCNC(=O)c3cnc4[nH]ccc4c3)cc1)C2 ; CC(C)[C@H](C(=O)N1CCC2(CC1)Oc1ccc(C#CC(=O)NCCCCn3ccc4cc(C(=O)NCCc5ccc(N6C[C@H]7CC[C@@H](C6)N7)cc5)cnc43)cc1O2)c1ccc(Cl)cc1]

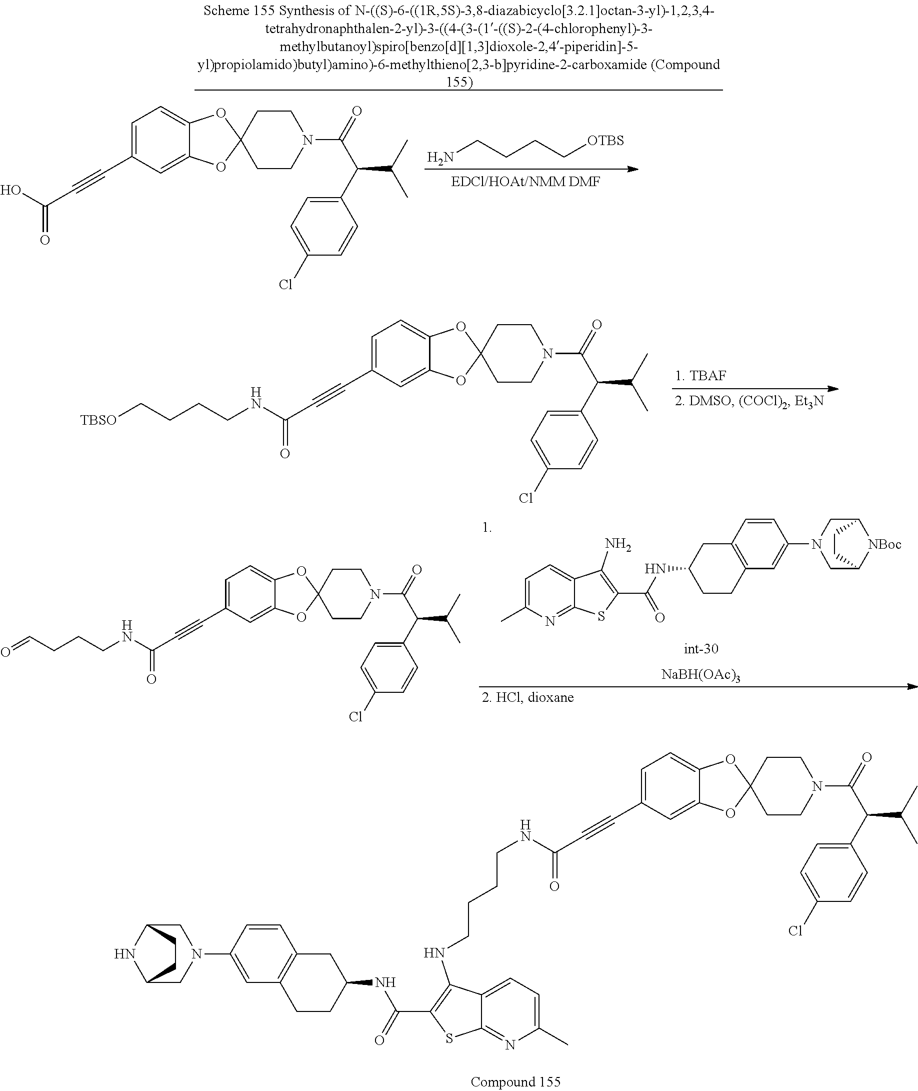

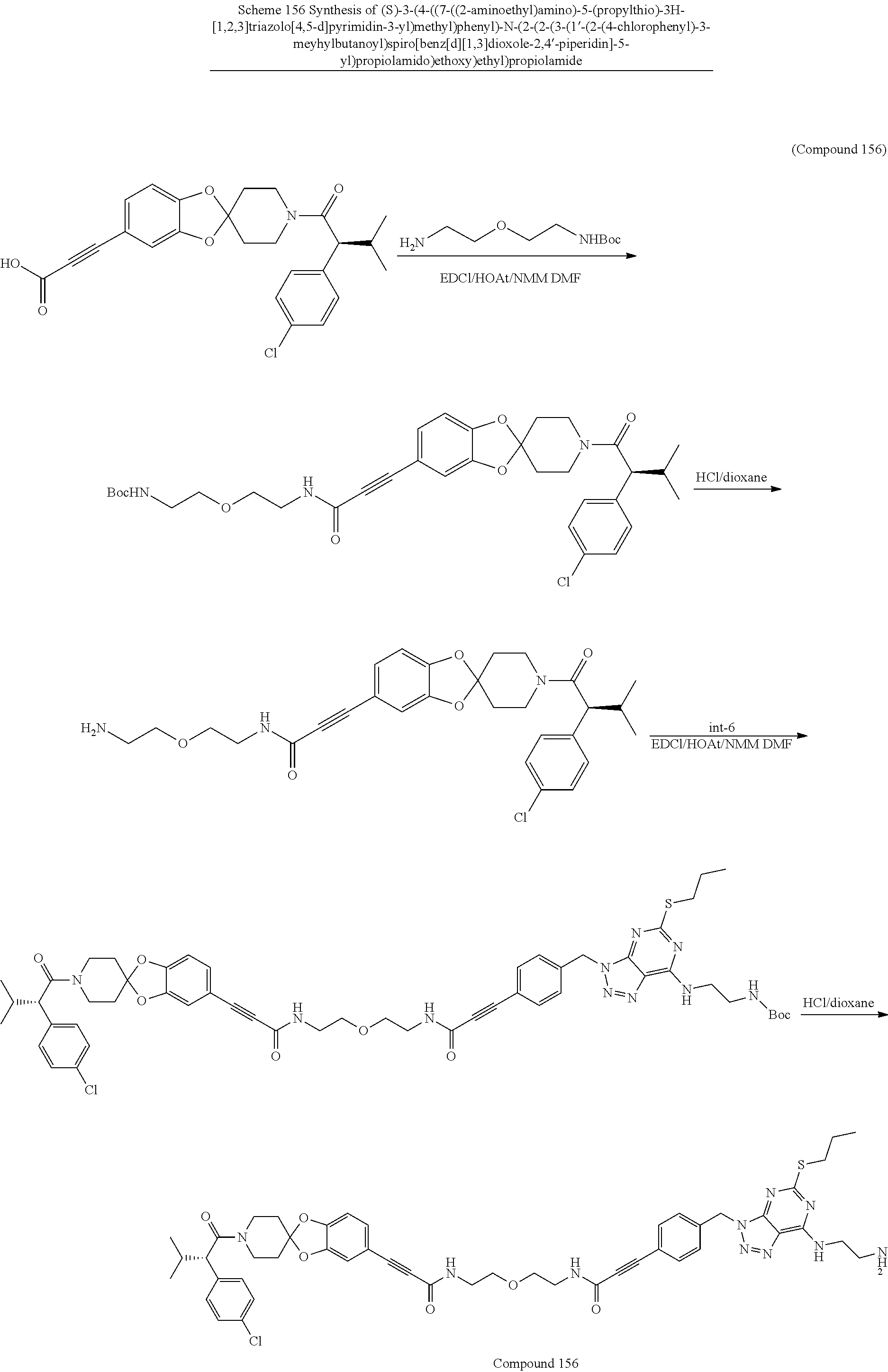

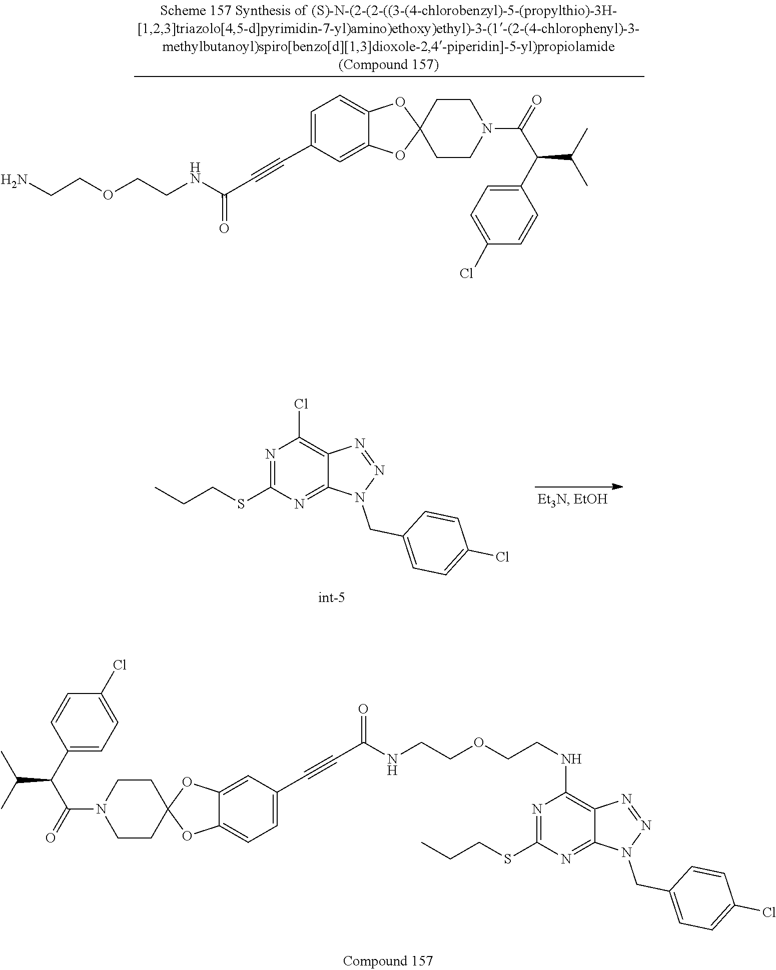

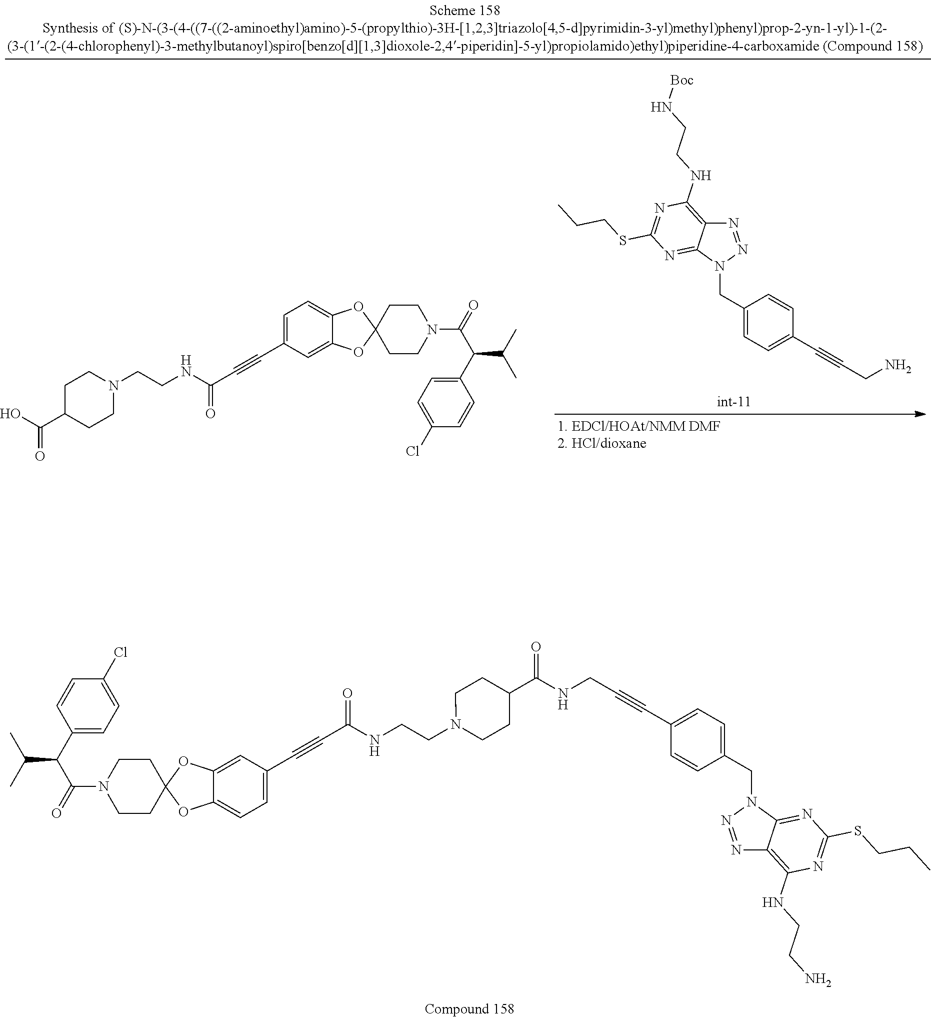 [SMILES: CC(C)[C@H](C(=O)N1CCC2(CC1)Oc1ccc(C#CC(=O)NCCN3CCC(C(=O)O)CC3)cc1O2)c1ccc(Cl)cc1 ; CCCSc1nc(NCCNC(=O)OC(C)(C)C)c2nnn(Cc3ccc(C#CCN)cc3)c2n1 ; CCCSc1nc(NCCN)c2nnn(Cc3ccc(C#CCNC(=O)C4CCN(CCNC(=O)C#Cc5ccc6c(c5)OC5(CCN(C(=O)[C@H](c7ccc(Cl)cc7)C(C)C)CC5)O6)CC4)cc3)c2n1]

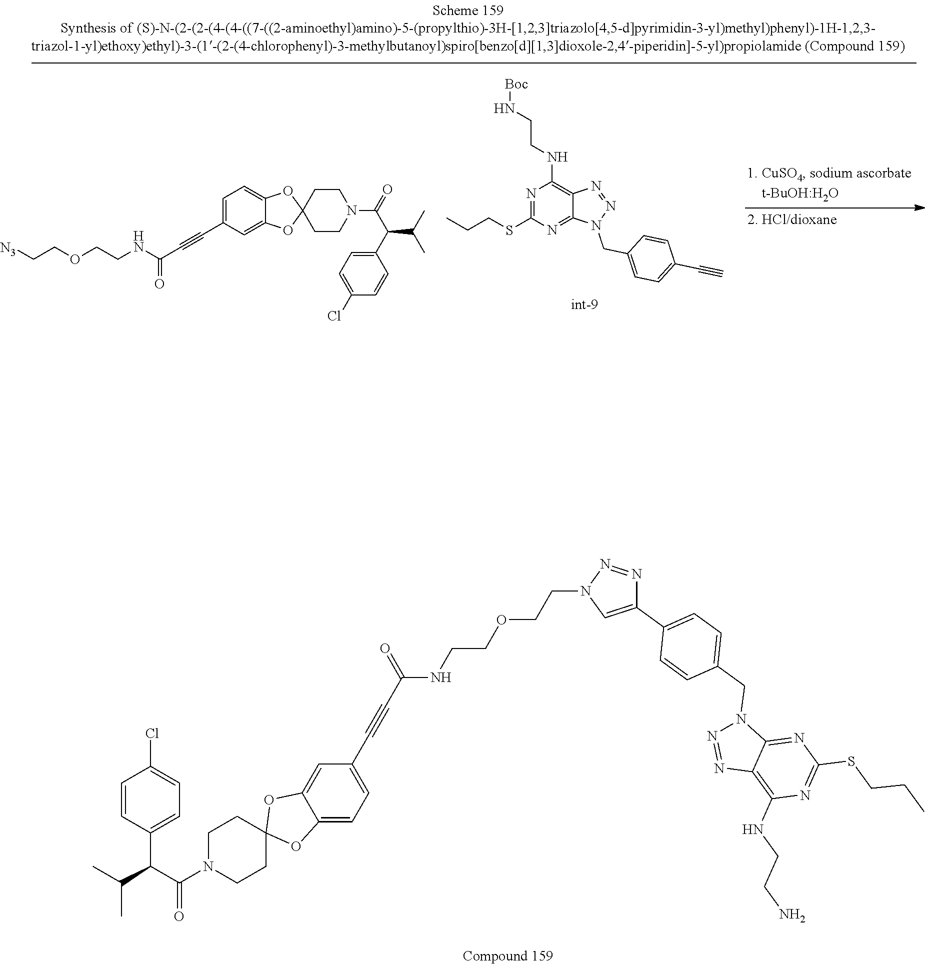

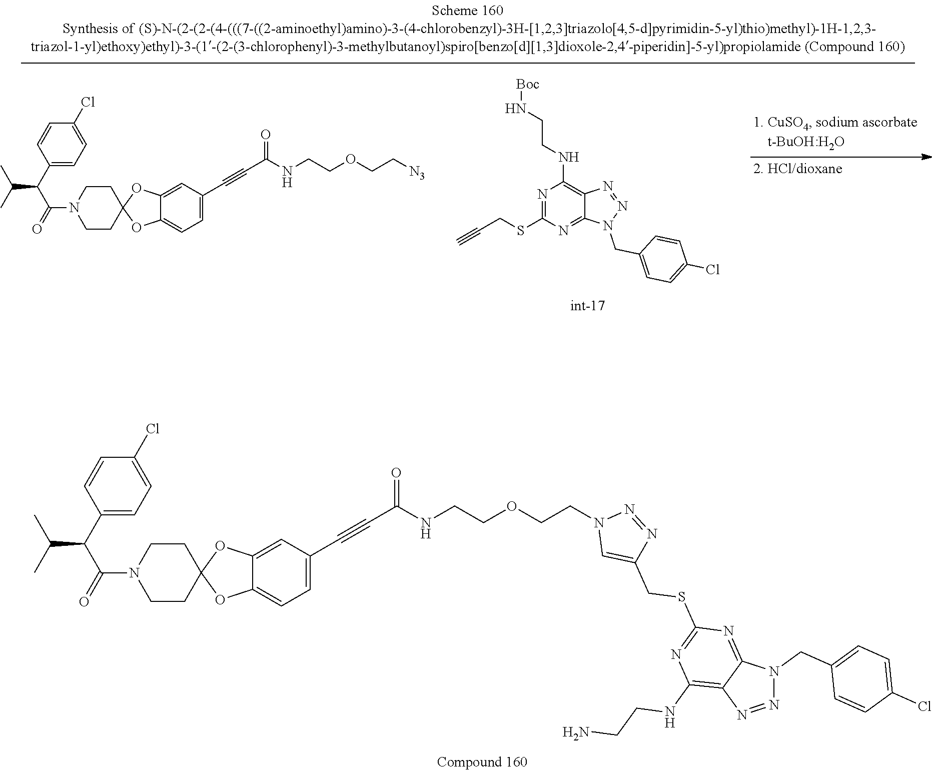 [SMILES: CC(C)[C@H](C(=O)N1CCC2(CC1)Oc1ccc(C#CC(=O)NCCOCCN=[N+]=[N-])cc1O2)c1ccc(Cl)cc1 ; C#CCSc1nc(NCCNC(=O)OC(C)(C)C)c2nnn(Cc3ccc(Cl)cc3)c2n1 ; CC(C)[C@H](C(=O)N1CCC2(CC1)Oc1ccc(C#CC(=O)NCCOCCn3cc(CSc4nc(NCCN)c5nnn(Cc6ccc(Cl)cc6)c5n4)nn3)cc1O2)c1ccc(Cl)cc1]

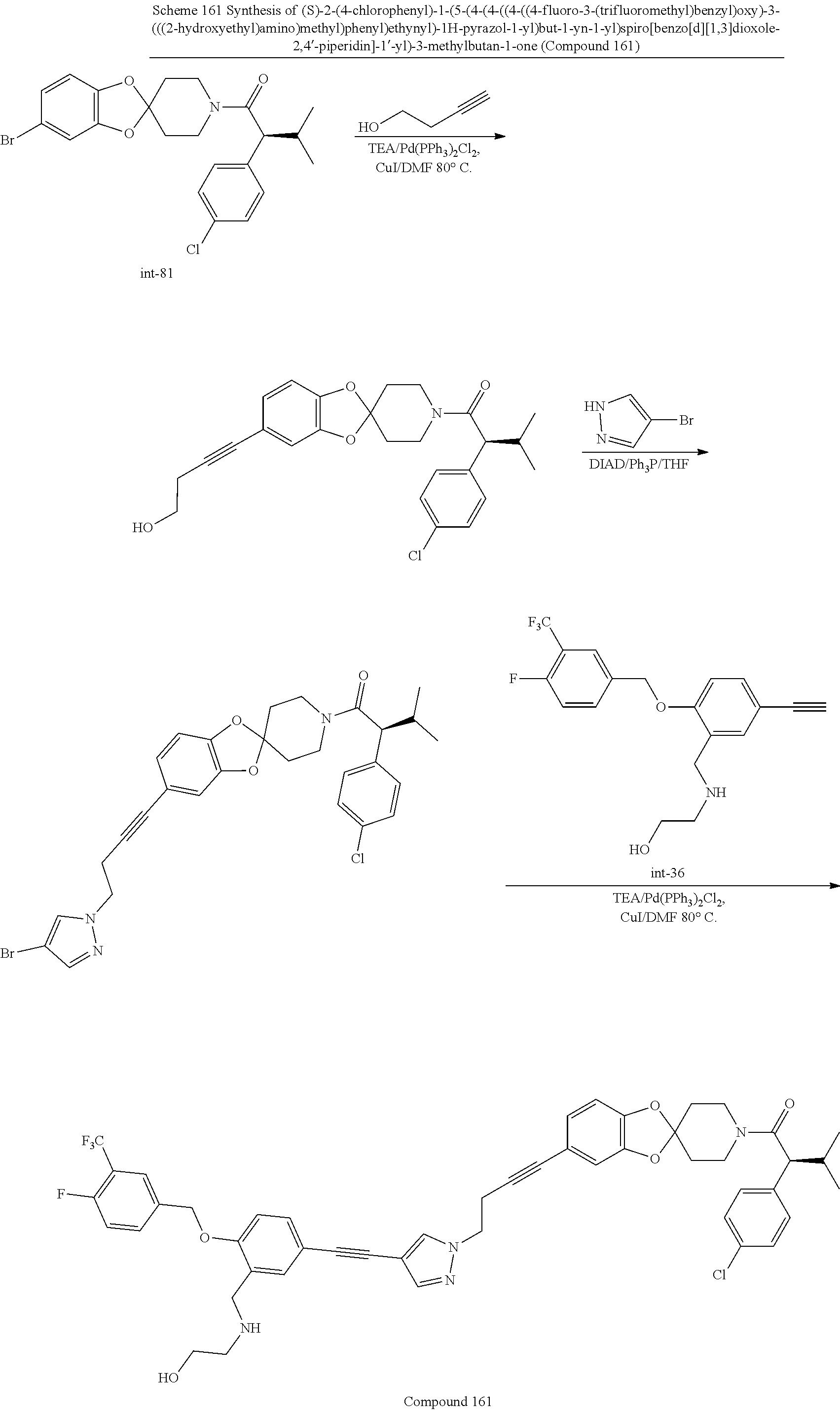

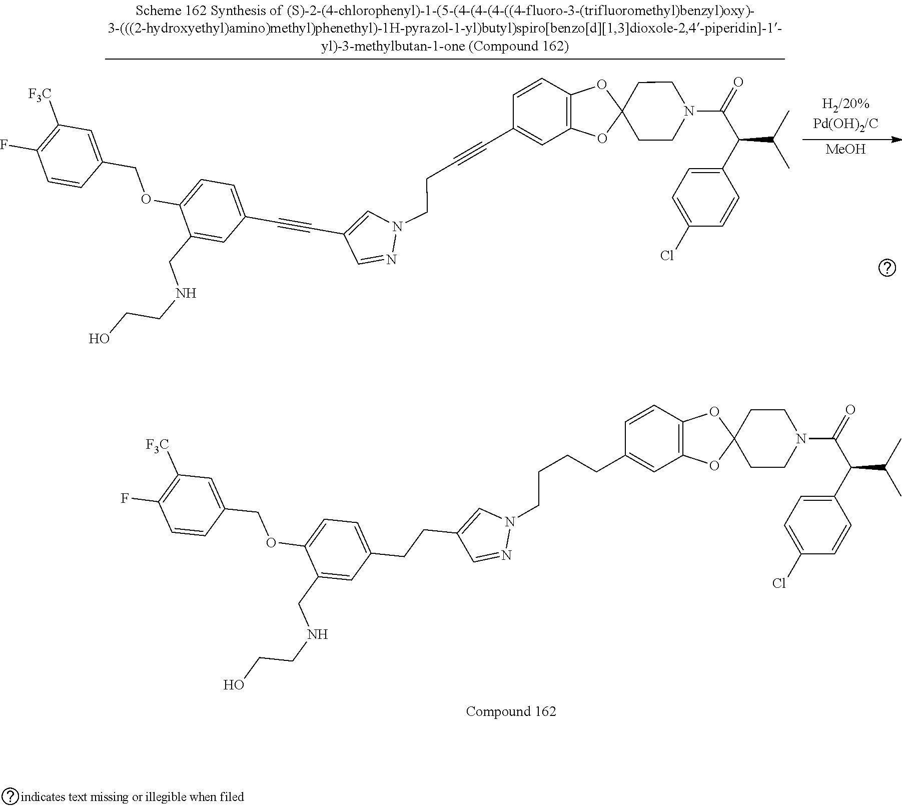 [SMILES: "MeOH" ; CC(C)[C@H](C(=O)N1CCC2(CC1)Oc1ccc(C#CCCn3cc(C#Cc4ccc(OCc5ccc(F)c(C(F)(F)F)c5)c(CNCCO)c4)cn3)cc1O2)c1ccc(Cl)cc1 ; CC(C)[C@H](C(=O)N1CCC2(CC1)Oc1ccc(CCCCn3cc(CCc4ccc(OCc5ccc(F)c(C(F)(F)F)c5)c(CNCCO)c4)cn3)cc1O2)c1ccc(Cl)cc1]

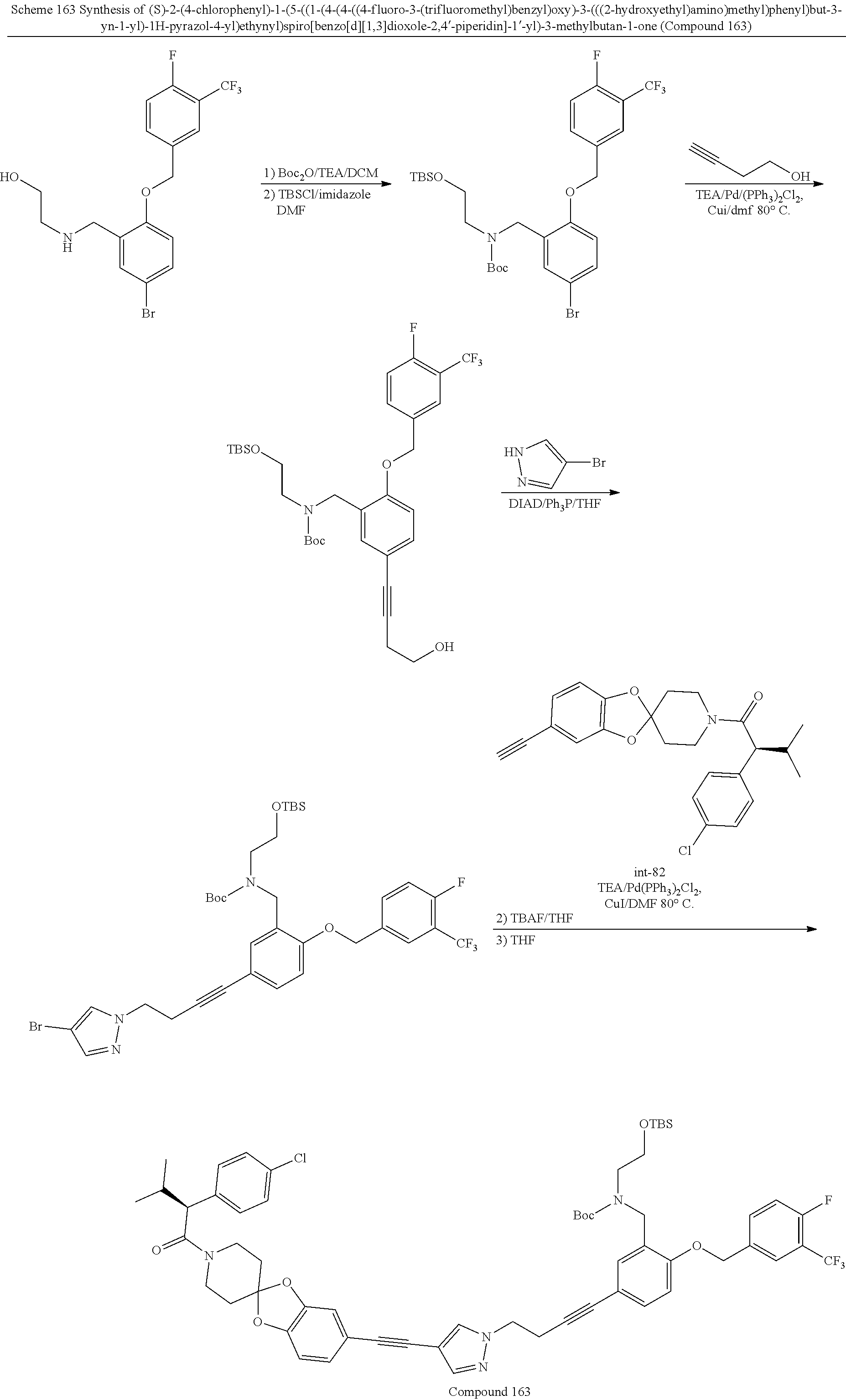

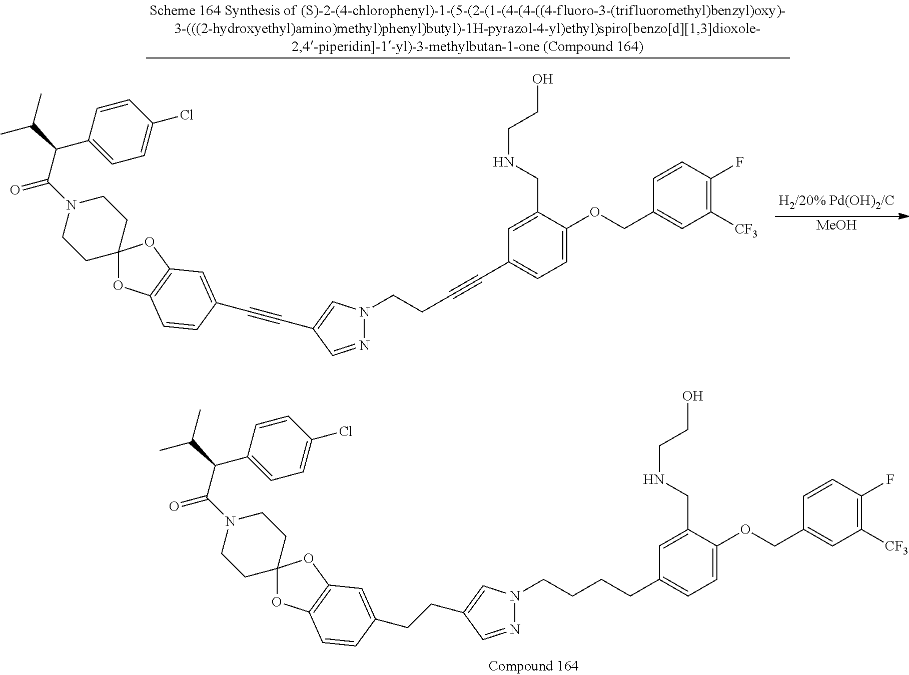 [SMILES: "MeOH" ; CC(C)[C@H](C(=O)N1CCC2(CC1)Oc1ccc(C#Cc3cnn(CCC#Cc4ccc(OCc5ccc(F)c(C(F)(F)F)c5)c(CNCCO)c4)c3)cc1O2)c1ccc(Cl)cc1 ; CC(C)[C@H](C(=O)N1CCC2(CC1)Oc1ccc(CCc3cnn(CCCCc4ccc(OCc5ccc(F)c(C(F)(F)F)c5)c(CNCCO)c4)c3)cc1O2)c1ccc(Cl)cc1]

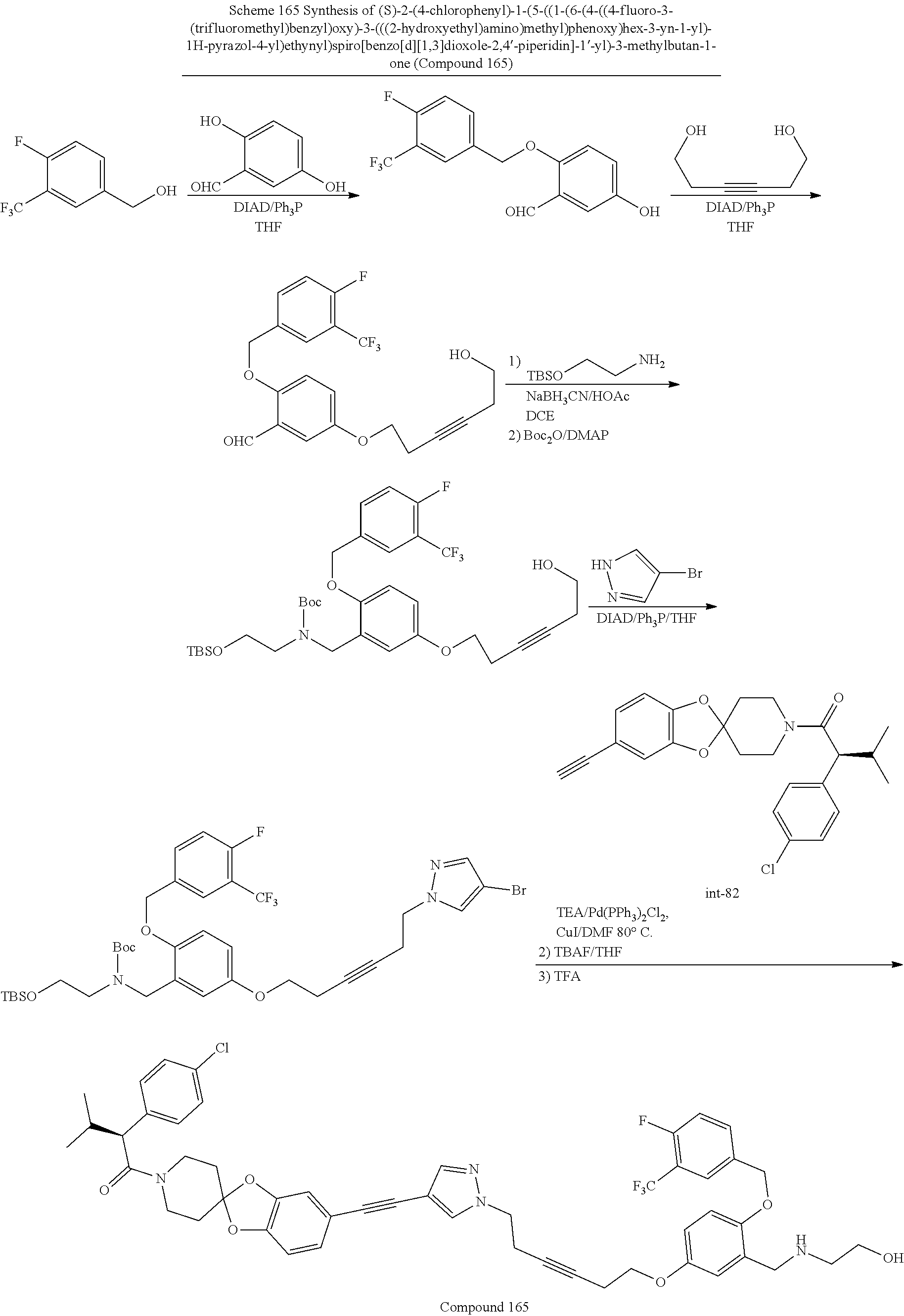

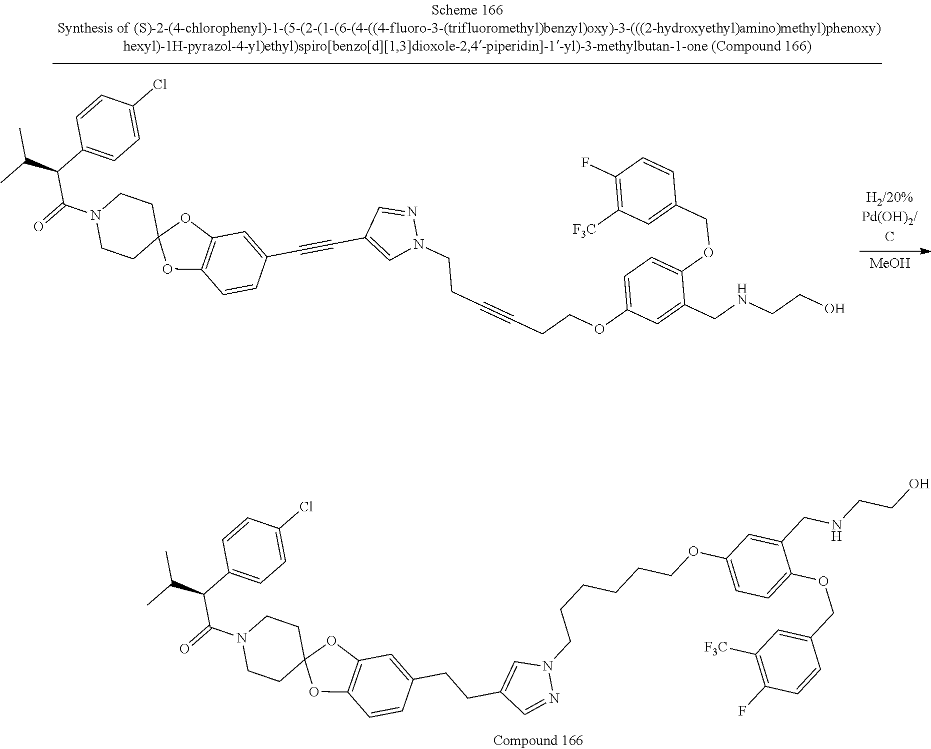 [SMILES: "MeOH" ; CC(C)[C@H](C(=O)N1CCC2(CC1)Oc1ccc(C#Cc3cnn(CCC#CCCOc4ccc(OCc5ccc(F)c(C(F)(F)F)c5)c(CNCCO)c4)c3)cc1O2)c1ccc(Cl)cc1 ; CC(C)[C@H](C(=O)N1CCC2(CC1)Oc1ccc(CCc3cnn(CCCCCCOc4ccc(OCc5ccc(F)c(C(F)(F)F)c5)c(CNCCO)c4)c3)cc1O2)c1ccc(Cl)cc1]

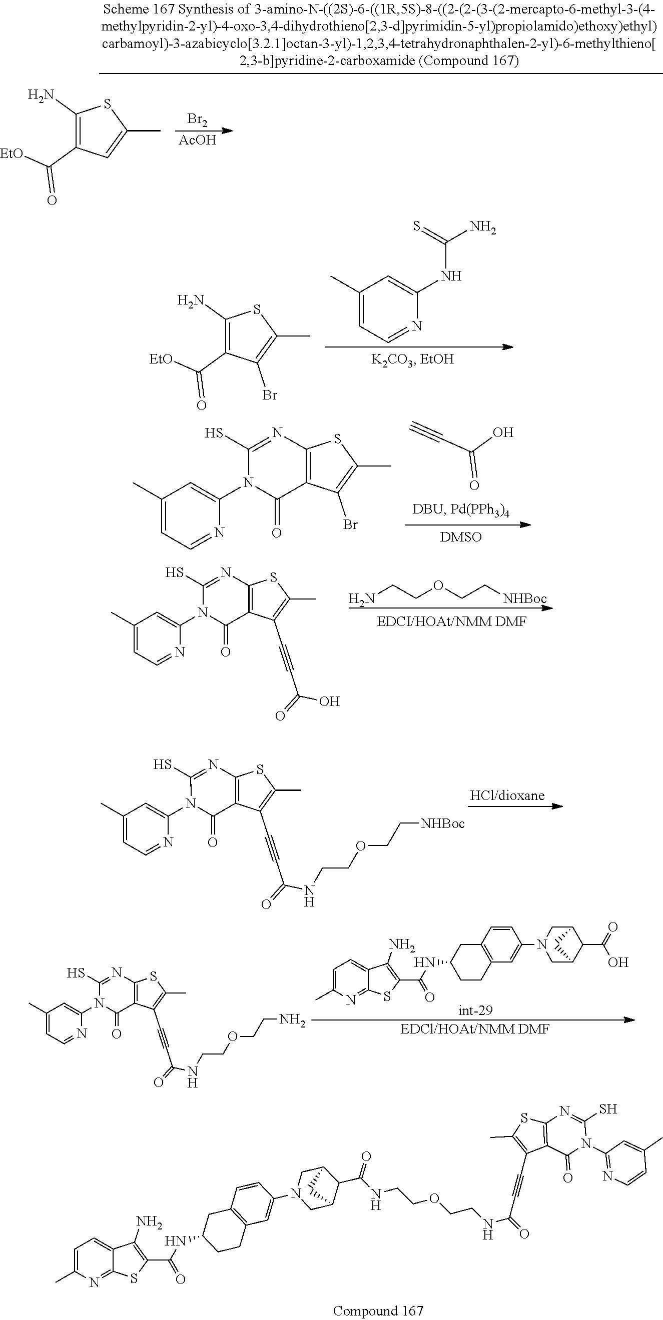

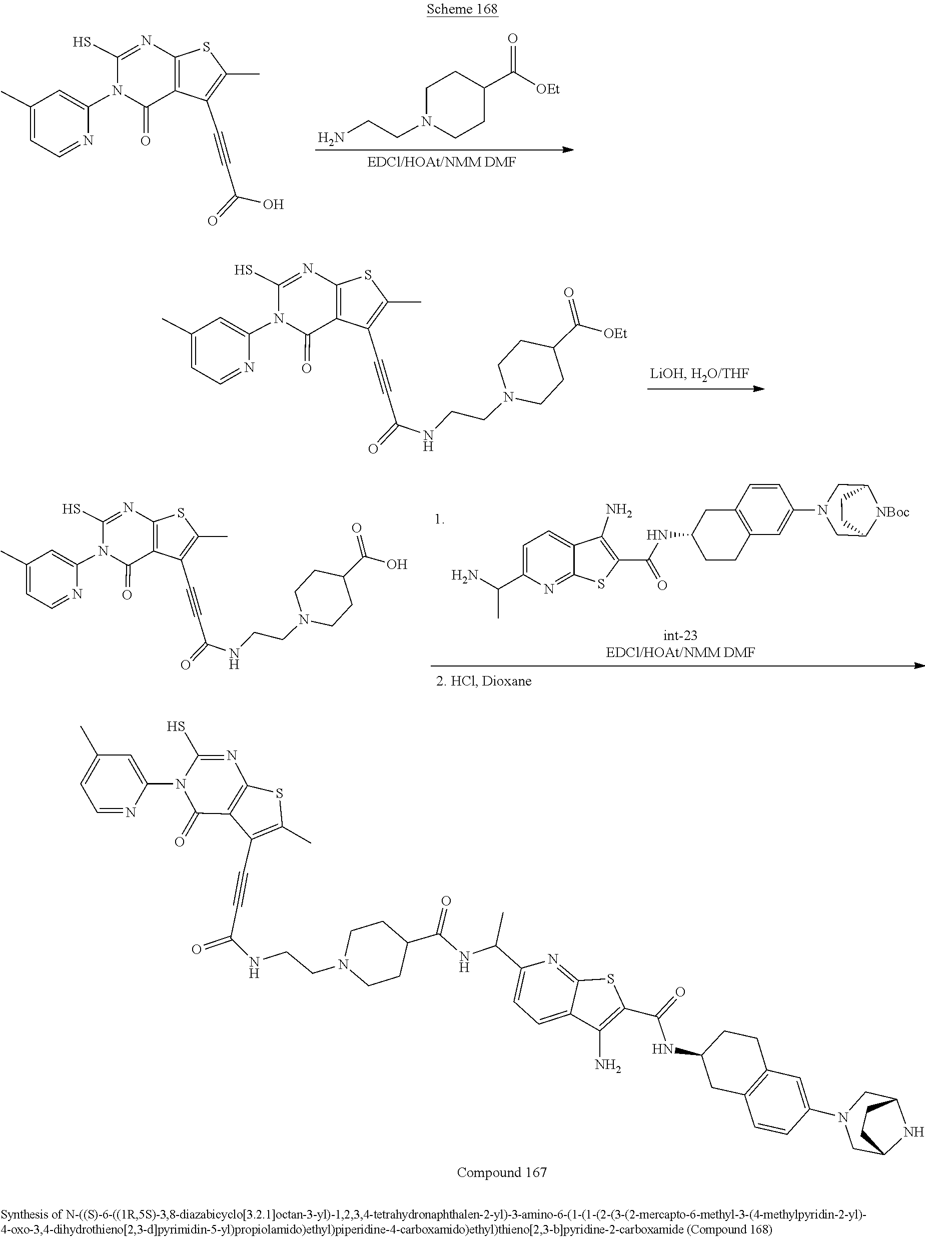 [SMILES: Cc1ccnc(-n2c(S)nc3sc(C)c(C#CC(=O)O)c3c2=O)c1 ; CCOC(=O)C1CCN(CCN)CC1 ; CCOC(=O)C1CCN(CCNC(=O)C#Cc2c(C)sc3nc(S)n(-c4cc(C)ccn4)c(=O)c23)CC1 ; Cc1ccnc(-n2c(S)nc3sc(C)c(C#CC(=O)NCCN4CCC(C(=O)O)CC4)c3c2=O)c1 ; CC(N)c1ccc2c(N)c(C(=O)N[C@H]3CCc4cc(N5C[C@H]6CC[C@@H](C5)N6C(=O)OC(C)(C)C)ccc4C3)sc2n1 ; Cc1ccnc(-n2c(S)nc3sc(C)c(C#CC(=O)NCCN4CCC(C(=O)NC(C)c5ccc6c(N)c(C(=O)N[C@H]7CCc8cc(N9C[C@H]%10CC[C@@H](C9)N%10)ccc8C7)sc6n5)CC4)c3c2=O)c1]

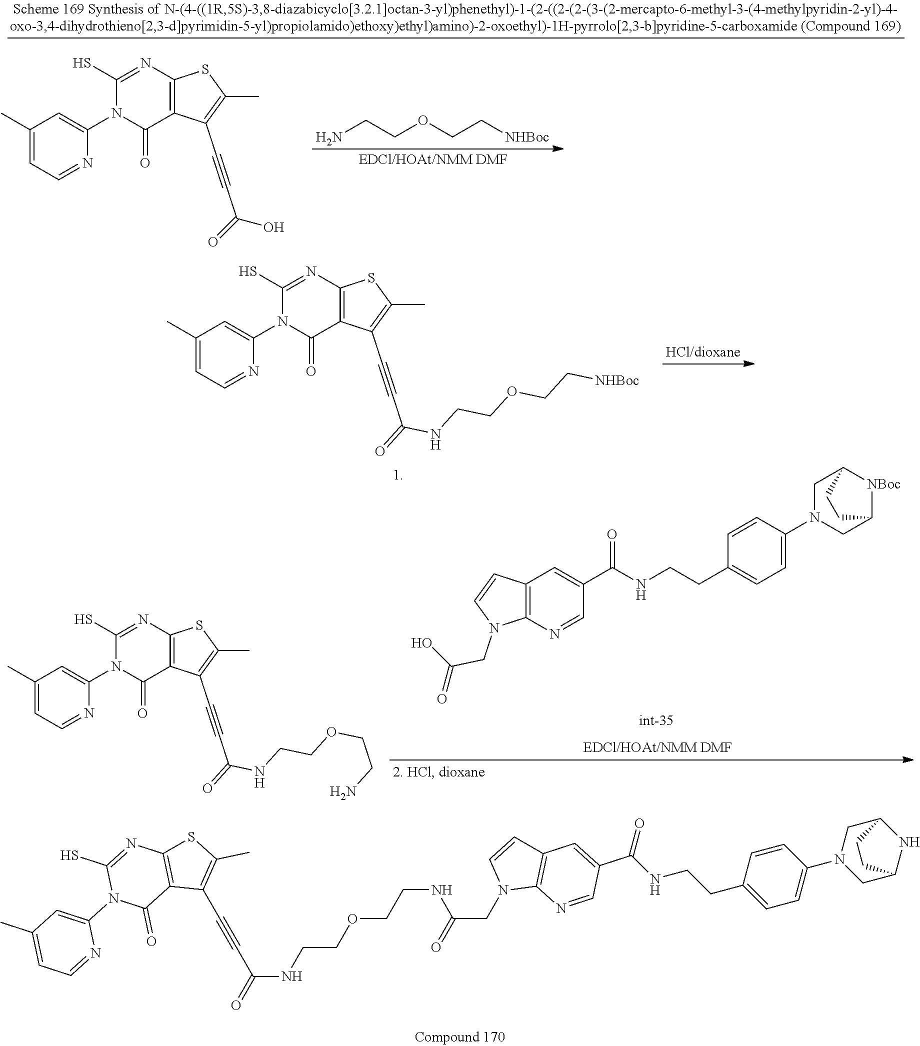 [SMILES: Cc1ccnc(-n2c(S)nc3sc(C)c(C#CC(=O)O)c3c2=O)c1 ; CC(C)(C)OC(=O)NCCOCCN ; Cc1ccnc(-n2c(S)nc3sc(C)c(C#CC(=O)NCCOCCNC(=O)OC(C)(C)C)c3c2=O)c1 ; Cc1ccnc(-n2c(S)nc3sc(C)c(C#CC(=O)NCCOCCN)c3c2=O)c1 ; CC(C)(C)OC(=O)N1[C@@H]2CC[C@H]1CN(c1ccc(CCNC(=O)c3cnc4c(ccn4CC(=O)O)c3)cc1)C2 ; Cc1ccnc(-n2c(S)nc3sc(C)c(C#CC(=O)NCCOCCNC(=O)Cn4ccc5cc(C(=O)NCCc6ccc(N7C[C@H]8CC[C@@H](C7)N8)cc6)cnc54)c3c2=O)c1]

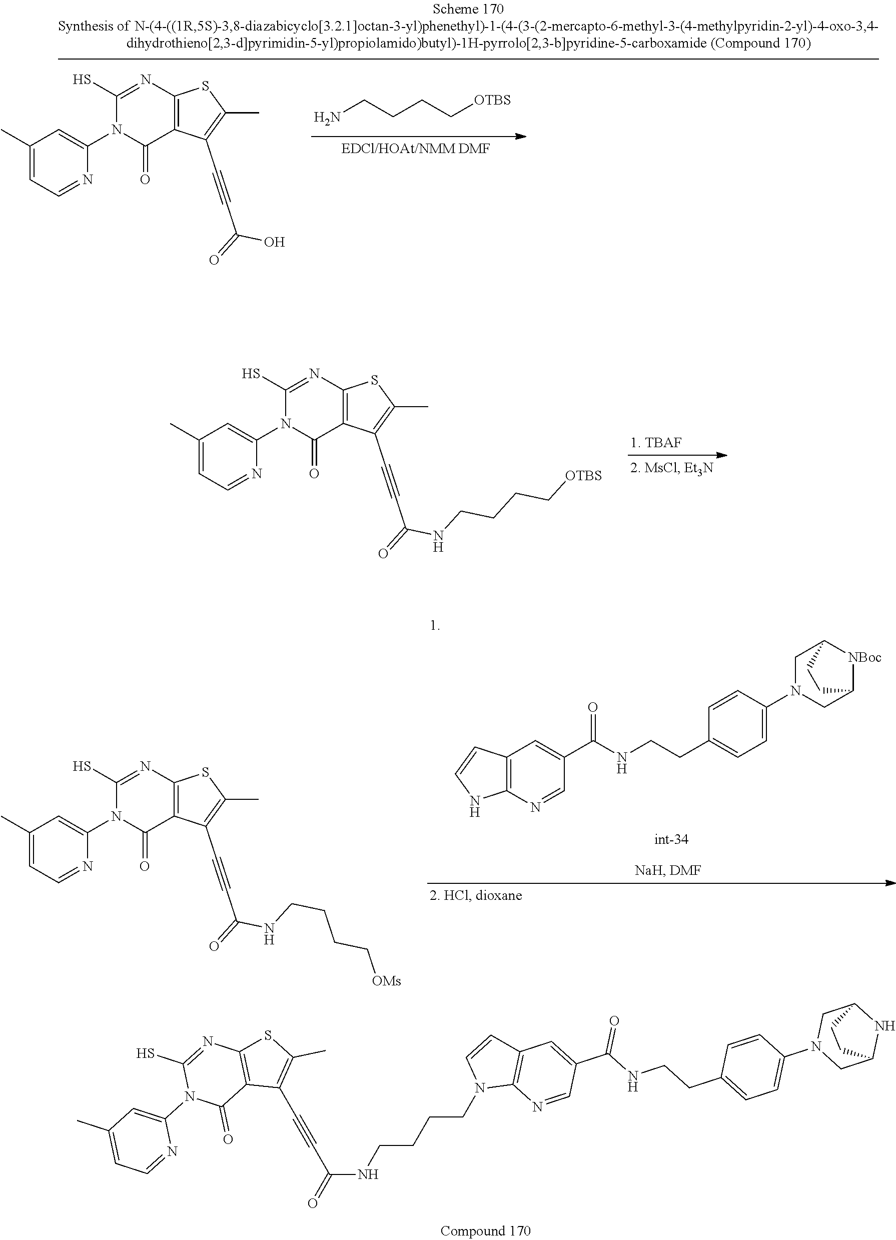 [SMILES: Cc1ccnc(-n2c(S)nc3sc(C)c(C#CC(=O)O)c3c2=O)c1 ; CC(C)(C)[Si](C)(C)OCCCCN ; Cc1ccnc(-n2c(S)nc3sc(C)c(C#CC(=O)NCCCCO[Si](C)(C)C(C)(C)C)c3c2=O)c1 ; Cc1ccnc(-n2c(S)nc3sc(C)c(C#CC(=O)NCCCCOS(C)(=O)=O)c3c2=O)c1 ; CC(C)(C)OC(=O)N1[C@@H]2CC[C@H]1CN(c1ccc(CCNC(=O)c3cnc4[nH]ccc4c3)cc1)C2 ; Cc1ccnc(-n2c(S)nc3sc(C)c(C#CC(=O)NCCCCn4ccc5cc(C(=O)NCCc6ccc(N7C[C@H]8CC[C@@H](C7)N8)cc6)cnc54)c3c2=O)c1]

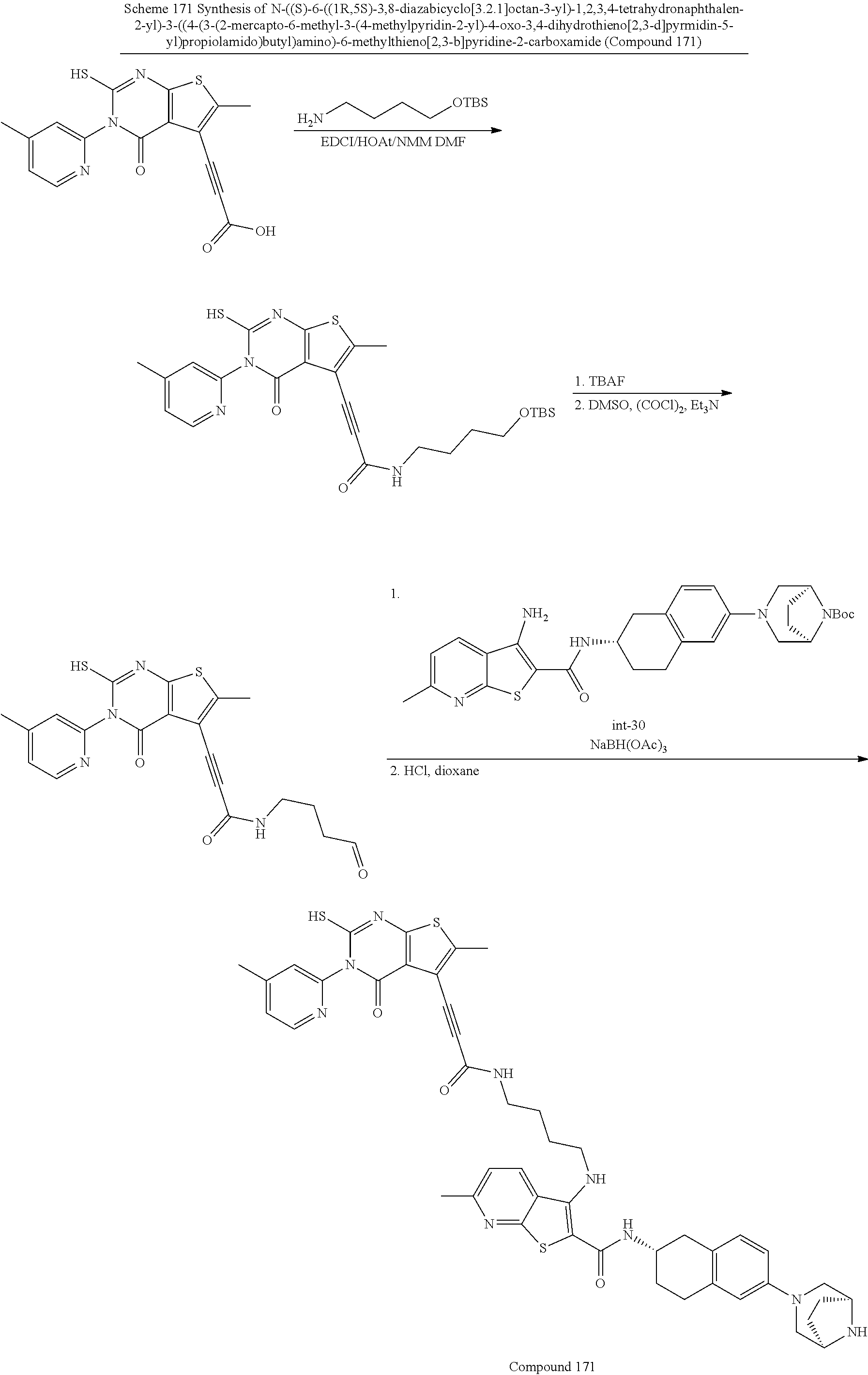

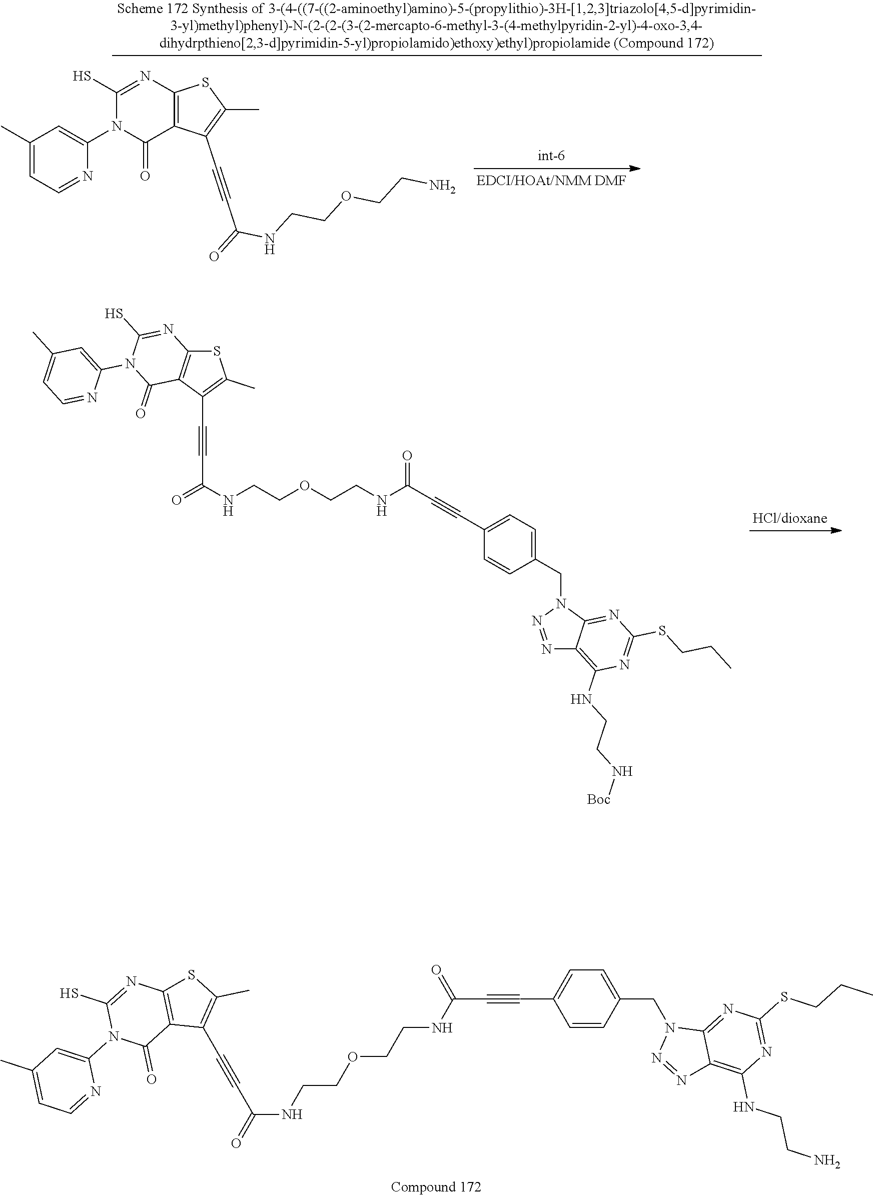

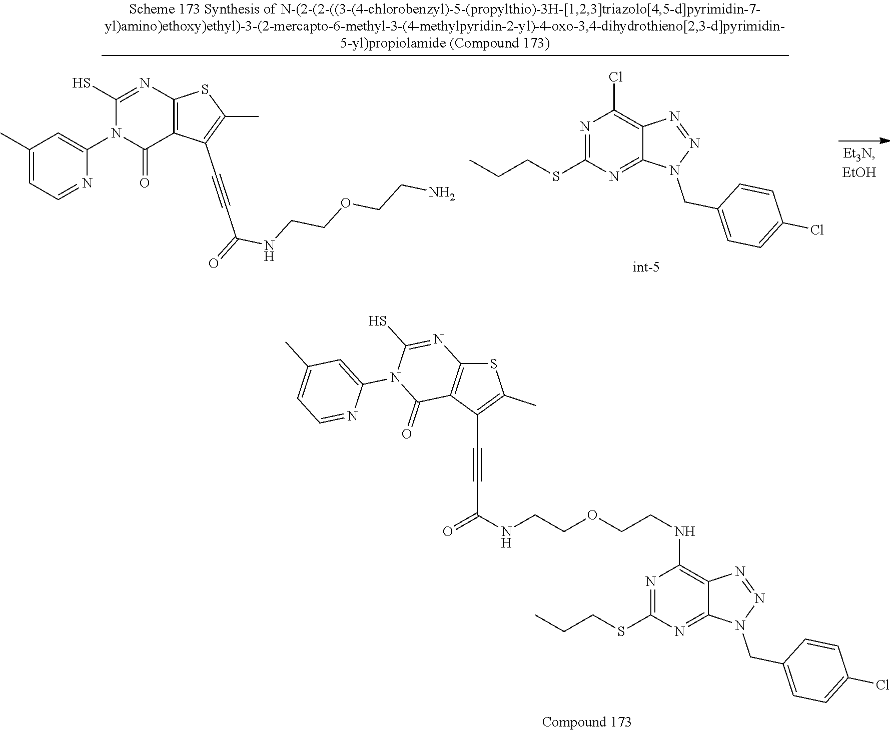 [SMILES: Cc1ccnc(-n2c(S)nc3sc(C)c(C#CC(=O)NCCOCCN)c3c2=O)c1 ; CCCSc1nc(Cl)c2nnn(Cc3ccc(Cl)cc3)c2n1 ; CCCSc1nc(NCCOCCNC(=O)C#Cc2c(C)sc3nc(S)n(-c4cc(C)ccn4)c(=O)c23)c2nnn(Cc3ccc(Cl)cc3)c2n1]

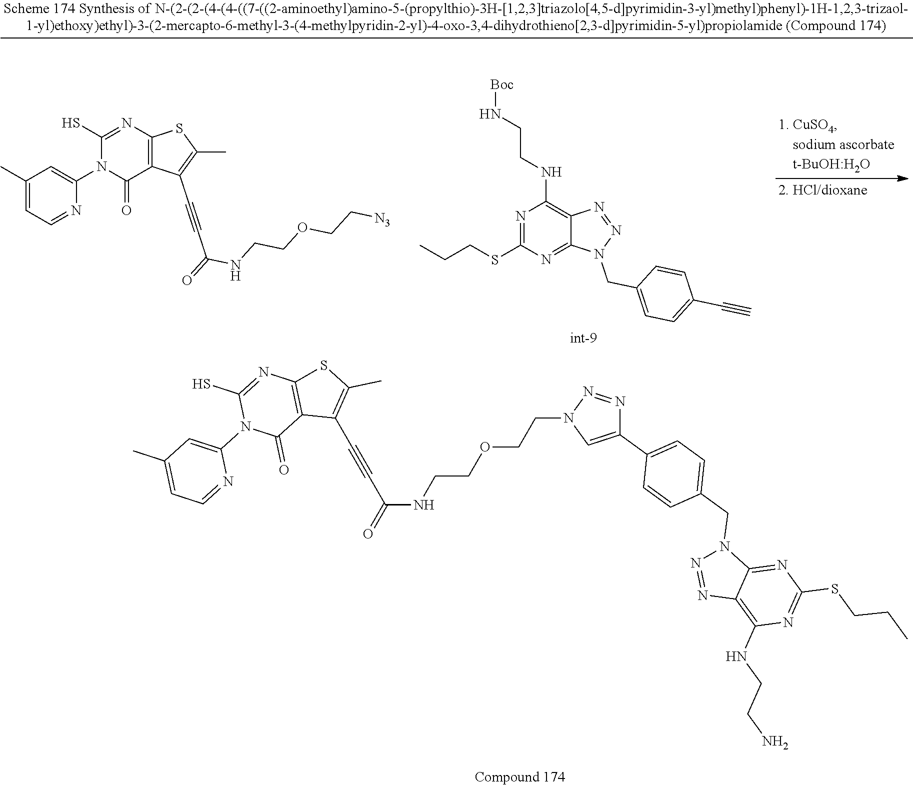 [SMILES: Cc1ccnc(-n2c(S)nc3sc(C)c(C#CC(=O)NCCOCCN=[N+]=[N-])c3c2=O)c1 ; C#Cc1ccc(Cn2nnc3c(NCCNC(=O)OC(C)(C)C)nc(SCCC)nc32)cc1 ; CCCSc1nc(NCCN)c2nnn(Cc3ccc(-c4cn(CCOCCNC(=O)C#Cc5c(C)sc6nc(S)n(-c7cc(C)ccn7)c(=O)c56)nn4)cc3)c2n1]

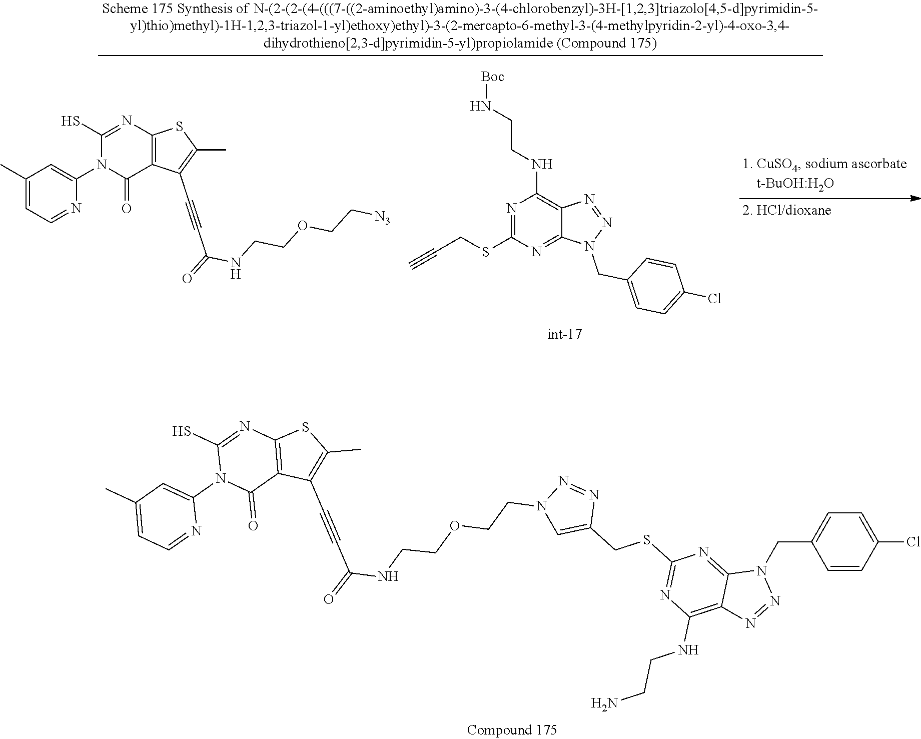 [SMILES: Cc1ccnc(-n2c(S)nc3sc(C)c(C#CC(=O)NCCOCCN=[N+]=[N-])c3c2=O)c1 ; C#CCSc1nc(NCCNC(=O)OC(C)(C)C)c2nnn(Cc3ccc(Cl)cc3)c2n1 ; Cc1ccnc(-n2c(S)nc3sc(C)c(C#CC(=O)NCCOCCn4cc(CSc5nc(NCCN)c6nnn(Cc7ccc(Cl)cc7)c6n5)nn4)c3c2=O)c1]

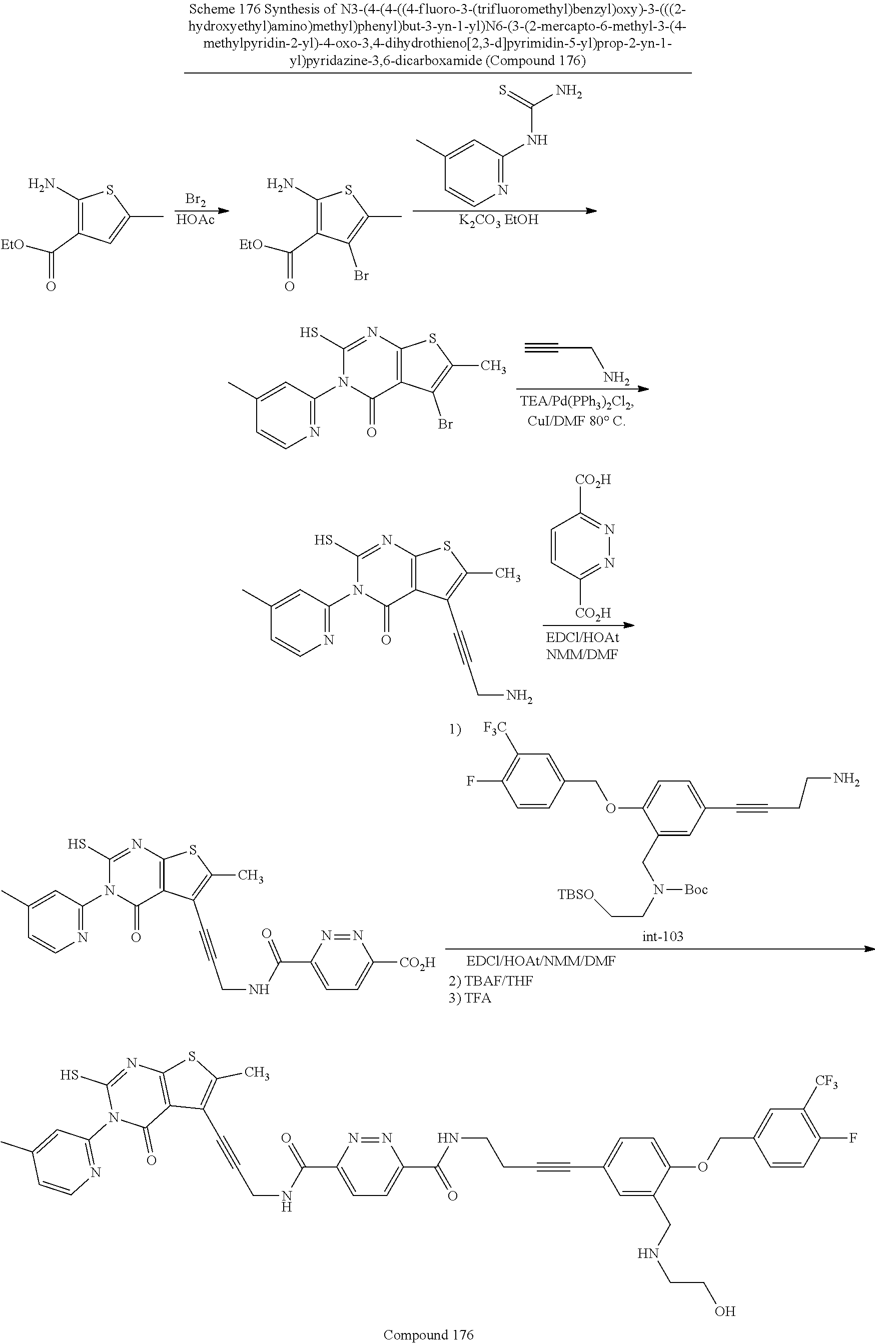

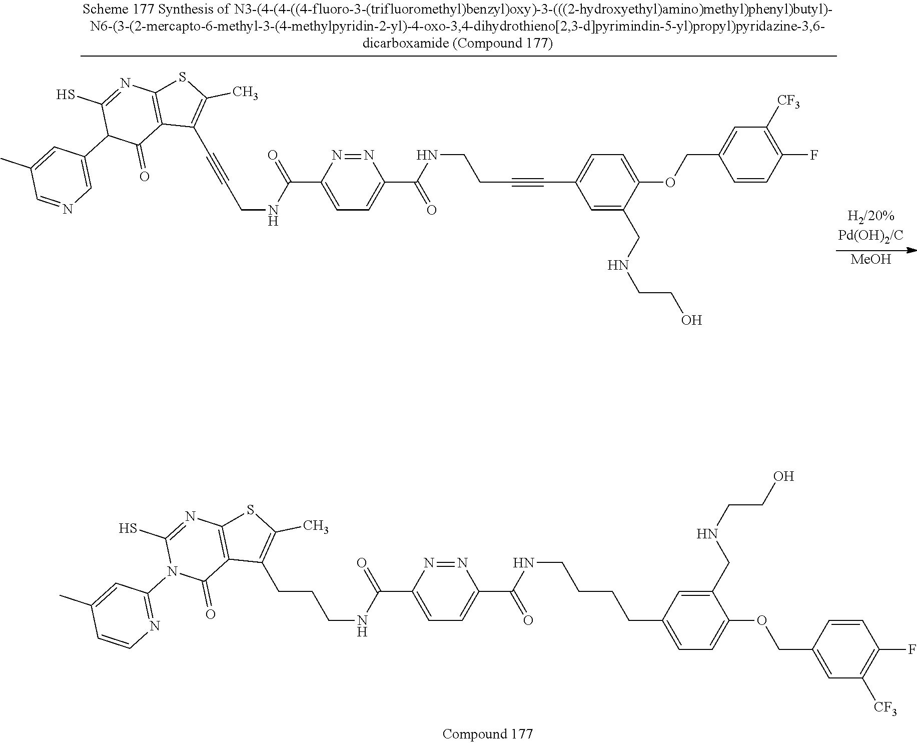 [SMILES: "MeOH" ; Cc1cncc(C2C(=O)c3c(sc(C)c3C#CCNC(=O)c3ccc(C(=O)NCCC#Cc4ccc(OCc5ccc(F)c(C(F)(F)F)c5)c(CNCCO)c4)nn3)N=C2S)c1 ; Cc1ccnc(-n2c(S)nc3sc(C)c(CCCNC(=O)c4ccc(C(=O)NCCCCc5ccc(OCc6ccc(F)c(C(F)(F)F)c6)c(CNCCO)c5)nn4)c3c2=O)c1]

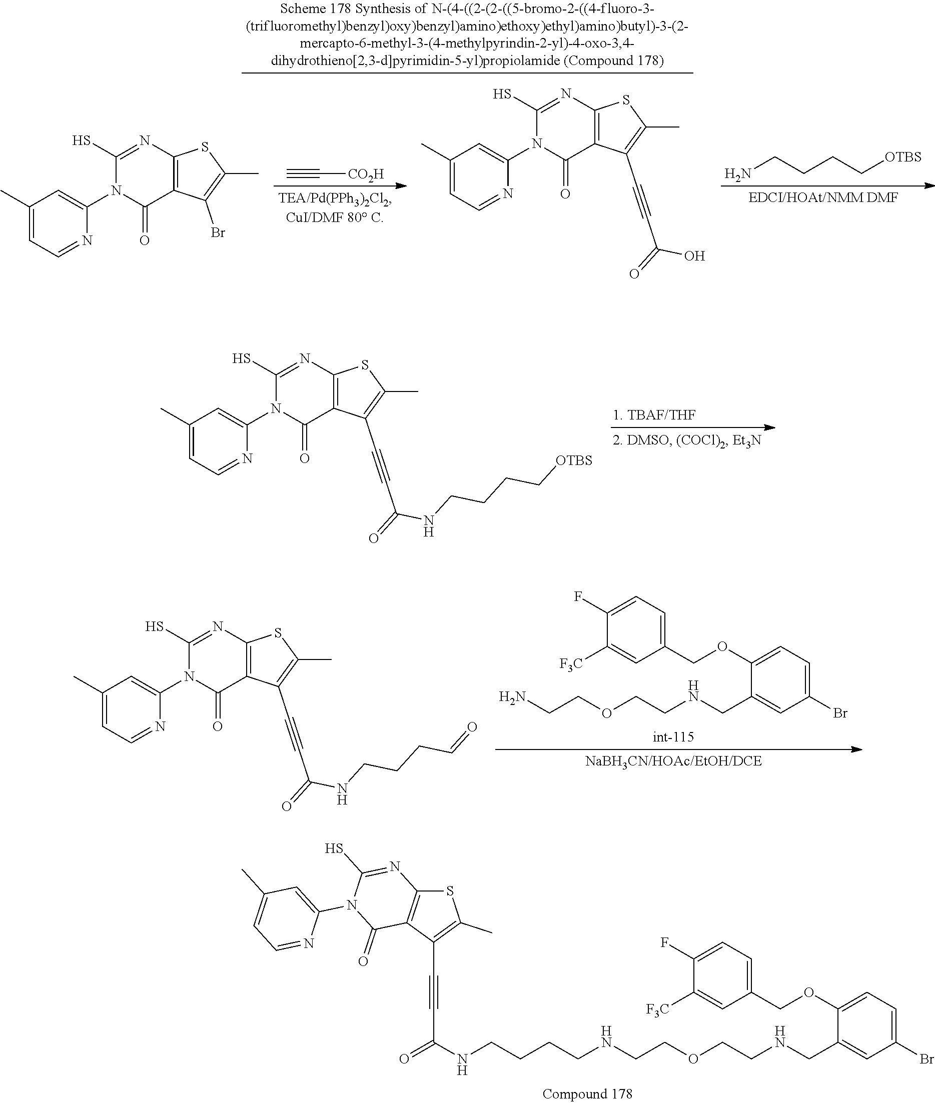

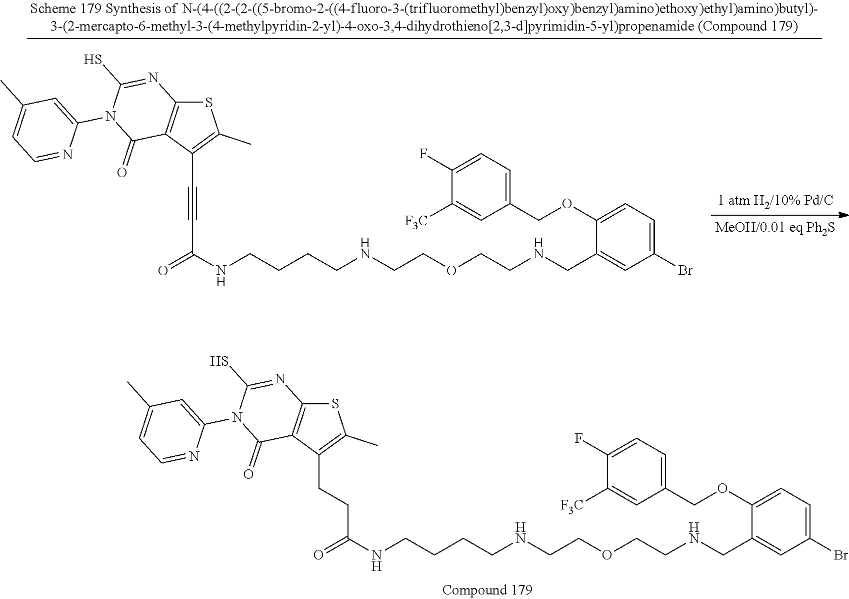 [SMILES: Cc1ccnc(-n2c(S)nc3sc(C)c(C#CC(=O)NCCCCNCCOCCNCc4cc(Br)ccc4OCc4ccc(F)c(C(F)(F)F)c4)c3c2=O)c1 ; Cc1ccnc(-n2c(S)nc3sc(C)c(CCC(=O)NCCCCNCCOCCNCc4cc(Br)ccc4OCc4ccc(F)c(C(F)(F)F)c4)c3c2=O)c1]

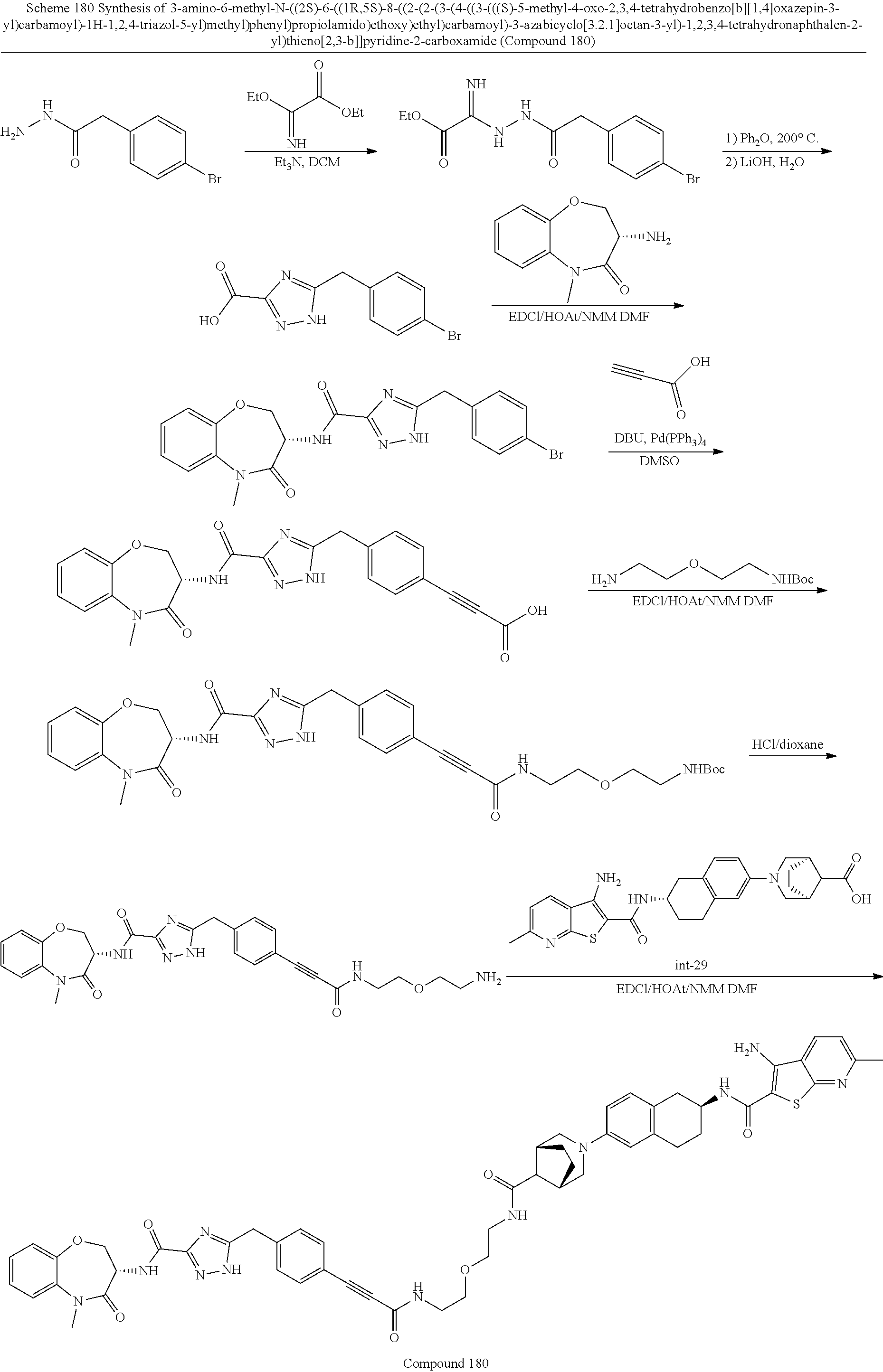

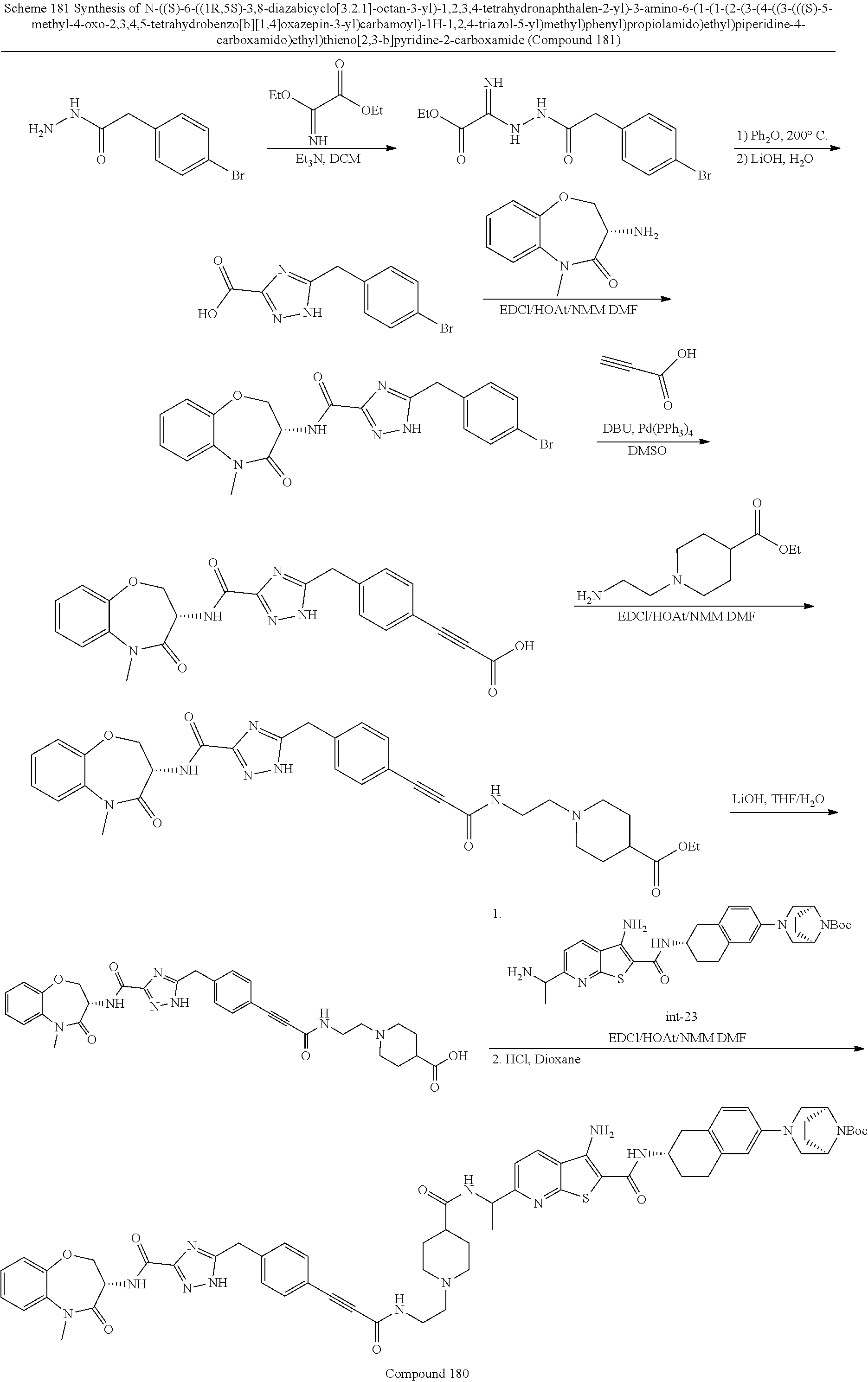

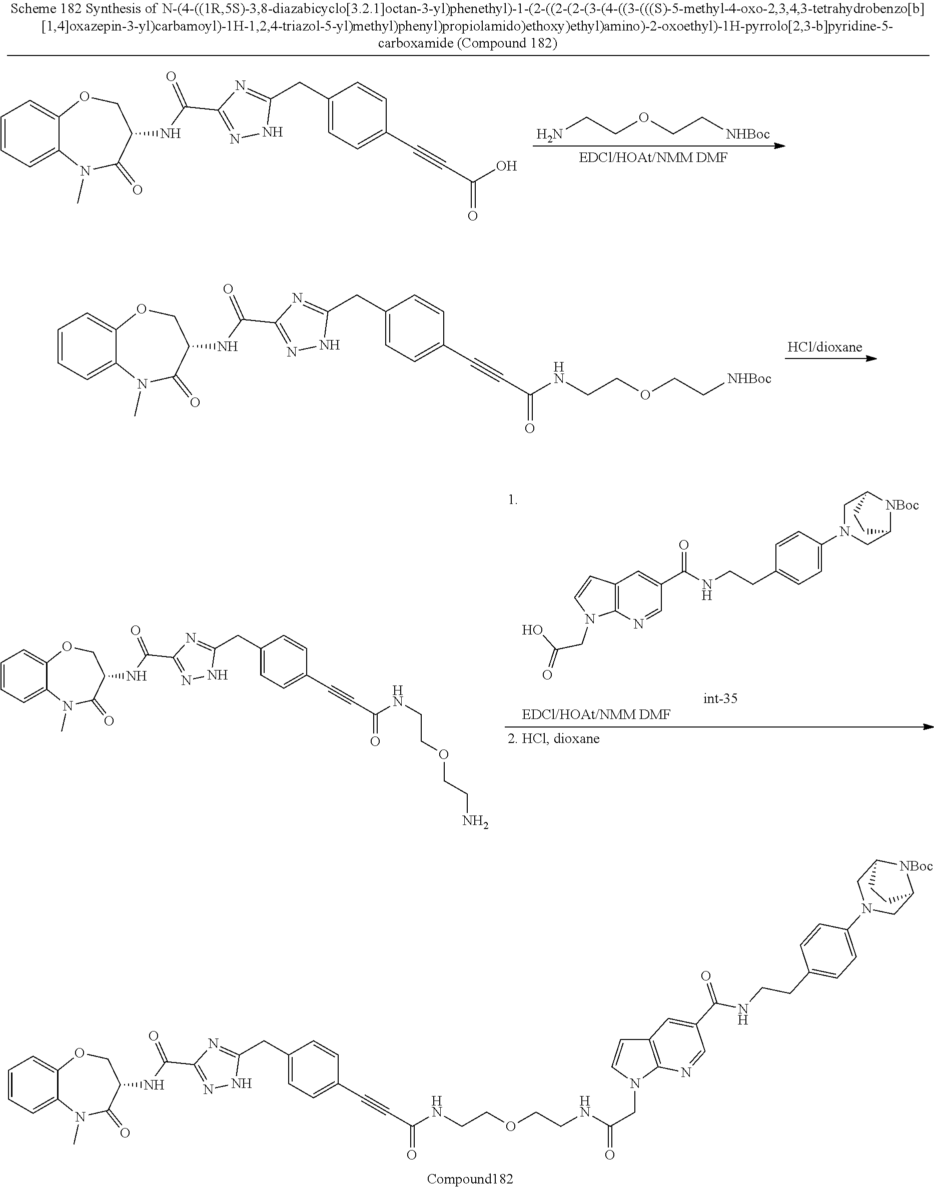 [SMILES: CN1C(=O)[C@@H](NC(=O)c2n[nH]c(Cc3ccc(C#CC(=O)O)cc3)n2)COc2ccccc21 ; CC(C)(C)OC(=O)NCCOCCN ; CN1C(=O)[C@@H](NC(=O)c2n[nH]c(Cc3ccc(C#CC(=O)NCCOCCNC(=O)OC(C)(C)C)cc3)n2)COc2ccccc21 ; CN1C(=O)[C@@H](NC(=O)c2n[nH]c(Cc3ccc(C#CC(=O)NCCOCCN)cc3)n2)COc2ccccc21 ; CC(C)(C)OC(=O)N1[C@@H]2CC[C@H]1CN(c1ccc(CCNC(=O)c3cnc4c(ccn4CC(=O)O)c3)cc1)C2 ; CN1C(=O)[C@@H](NC(=O)c2n[nH]c(Cc3ccc(C#CC(=O)NCCOCCNC(=O)Cn4ccc5cc(C(=O)NCCc6ccc(N7C[C@H]8CC[C@@H](C7)N8C(=O)OC(C)(C)C)cc6)cnc54)cc3)n2)COc2ccccc21]

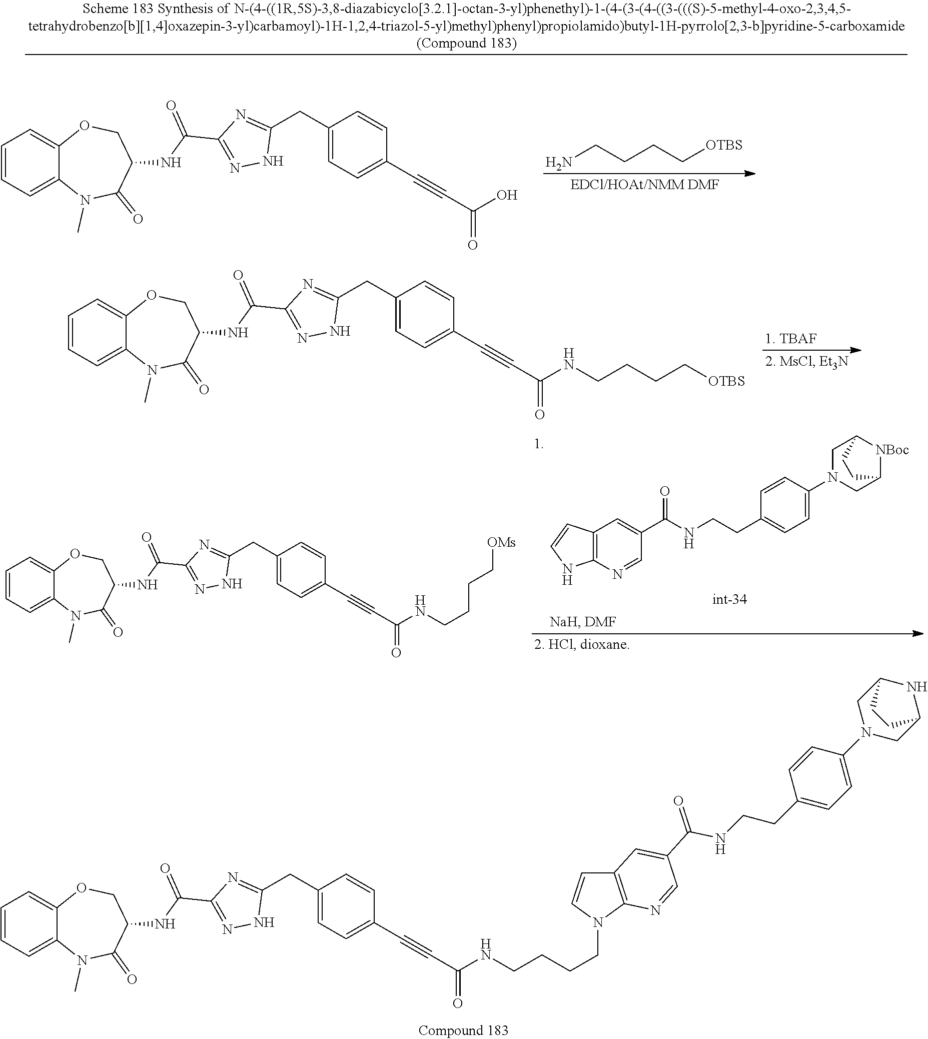 [SMILES: CN1C(=O)[C@@H](NC(=O)c2n[nH]c(Cc3ccc(C#CC(=O)O)cc3)n2)COc2ccccc21 ; CC(C)(C)[Si](C)(C)OCCCCN ; CN1C(=O)[C@@H](NC(=O)c2n[nH]c(Cc3ccc(C#CC(=O)NCCCCO[Si](C)(C)C(C)(C)C)cc3)n2)COc2ccccc21 ; CN1C(=O)[C@@H](NC(=O)c2n[nH]c(Cc3ccc(C#CC(=O)NCCCCOS(C)(=O)=O)cc3)n2)COc2ccccc21 ; CC(C)(C)OC(=O)N1[C@@H]2CC[C@H]1CN(c1ccc(CCNC(=O)c3cnc4[nH]ccc4c3)cc1)C2 ; CN1C(=O)[C@@H](NC(=O)c2n[nH]c(Cc3ccc(C#CC(=O)NCCCCn4ccc5cc(C(=O)NCCc6ccc(N7C[C@H]8CC[C@@H](C7)N8)cc6)cnc54)cc3)n2)COc2ccccc21]

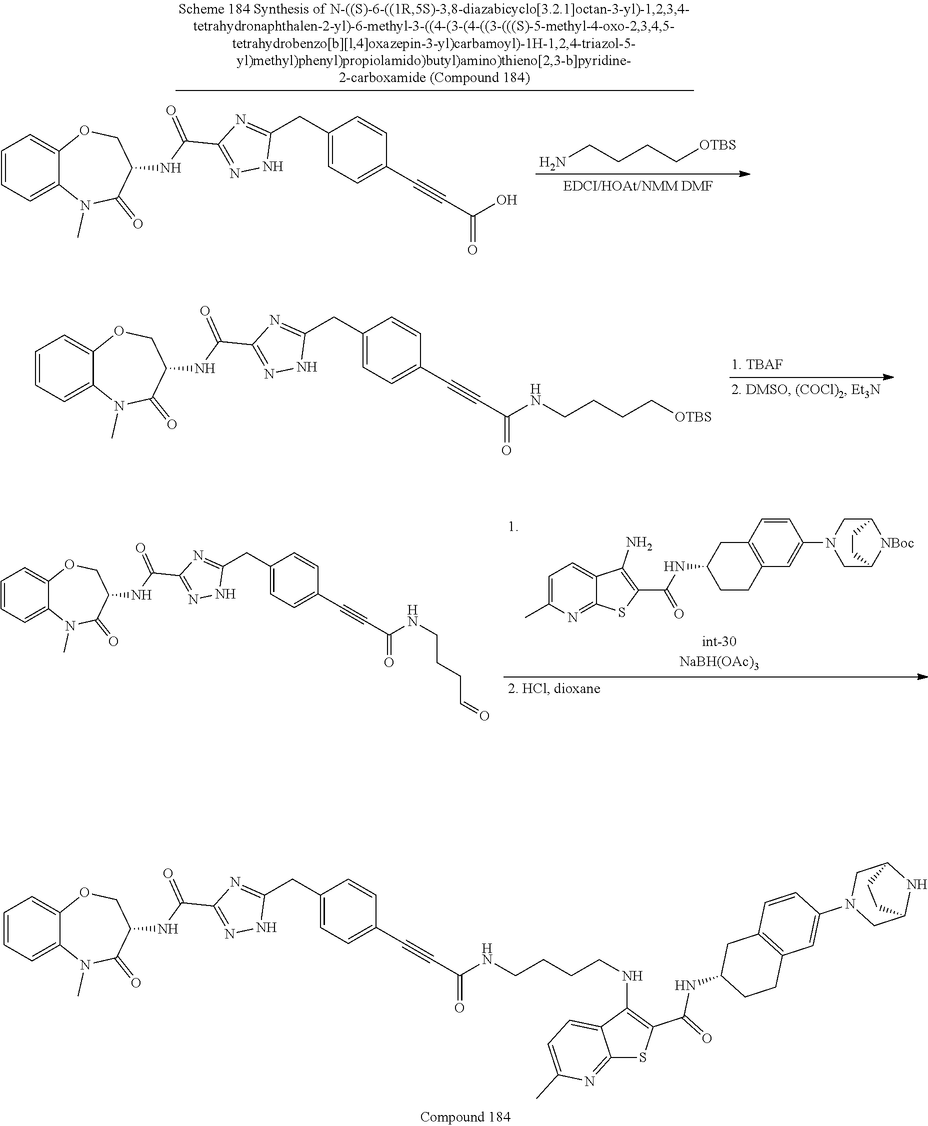 [SMILES: CN1C(=O)[C@@H](NC(=O)c2n[nH]c(Cc3ccc(C#CC(=O)O)cc3)n2)COc2ccccc21 ; CC(C)(C)[Si](C)(C)OCCCCN ; CN1C(=O)[C@@H](NC(=O)c2n[nH]c(Cc3ccc(C#CC(=O)NCCCCO[Si](C)(C)C(C)(C)C)cc3)n2)COc2ccccc21 ; CN1C(=O)[C@@H](NC(=O)c2n[nH]c(Cc3ccc(C#CC(=O)NCCCC=O)cc3)n2)COc2ccccc21 ; Cc1ccc2c(N)c(C(=O)N[C@H]3CCc4cc(N5C[C@H]6CC[C@@H](C5)N6C(=O)OC(C)(C)C)ccc4C3)sc2n1 ; Cc1ccc2c(NCCCCNC(=O)C#Cc3ccc(Cc4nc(C(=O)N[C@H]5COc6ccccc6N(C)C5=O)n[nH]4)cc3)c(C(=O)N[C@H]3CCc4cc(N5C[C@H]6CC[C@@H](C5)N6)ccc4C3)sc2n1]

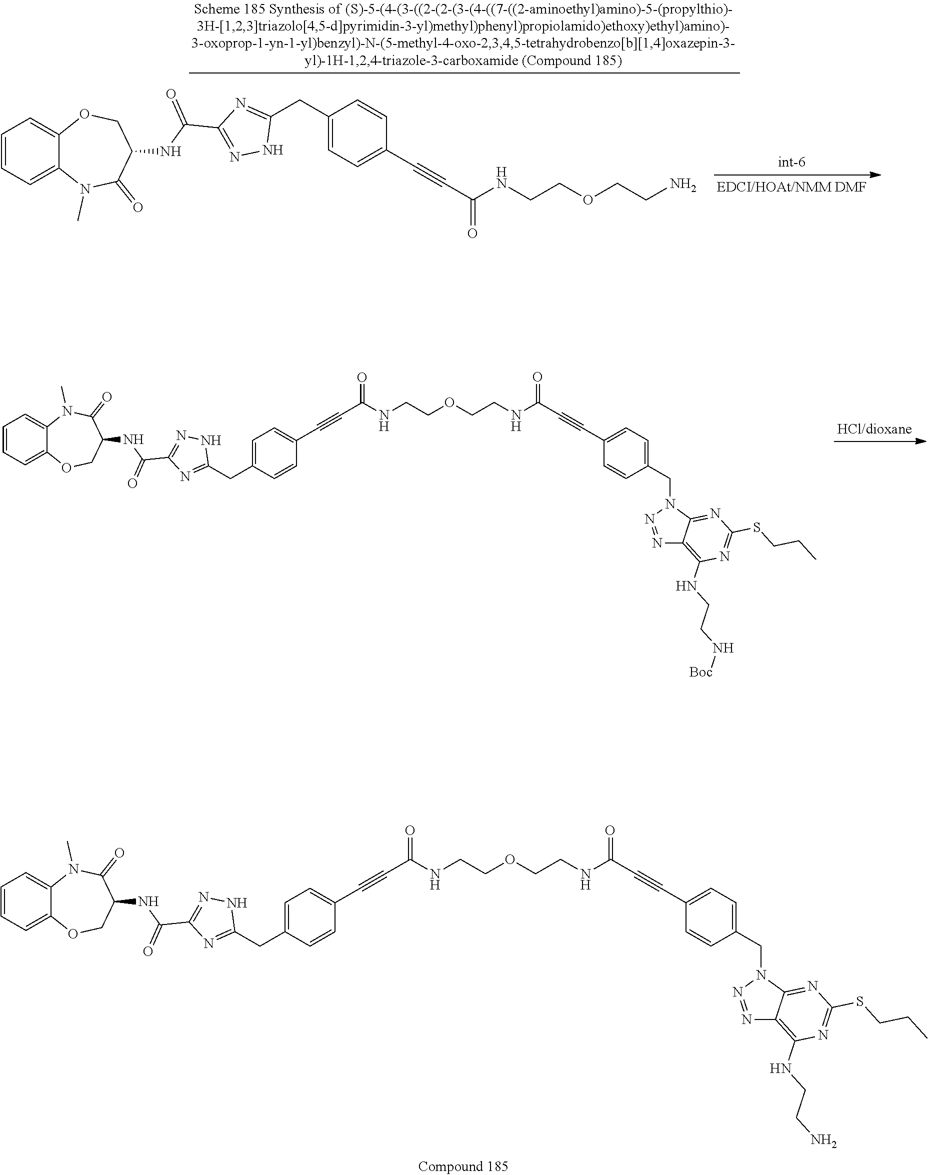 [SMILES: CN1C(=O)[C@@H](NC(=O)c2n[nH]c(Cc3ccc(C#CC(=O)NCCOCCN)cc3)n2)COc2ccccc21 ; CCCSc1nc(NCCNC(=O)OC(C)(C)C)c2nnn(Cc3ccc(C#CC(=O)NCCOCCNC(=O)C#Cc4ccc(Cc5nc(C(=O)N[C@H]6COc7ccccc7N(C)C6=O)n[nH]5)cc4)cc3)c2n1 ; CCCSc1nc(NCCN)c2nnn(Cc3ccc(C#CC(=O)NCCOCCNC(=O)C#Cc4ccc(Cc5nc(C(=O)N[C@H]6COc7ccccc7N(C)C6=O)n[nH]5)cc4)cc3)c2n1]

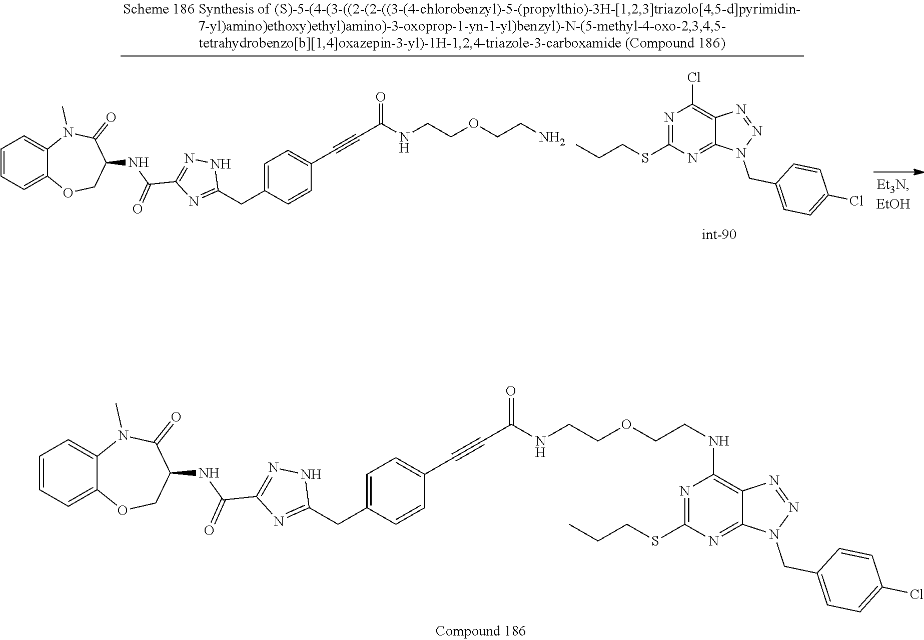

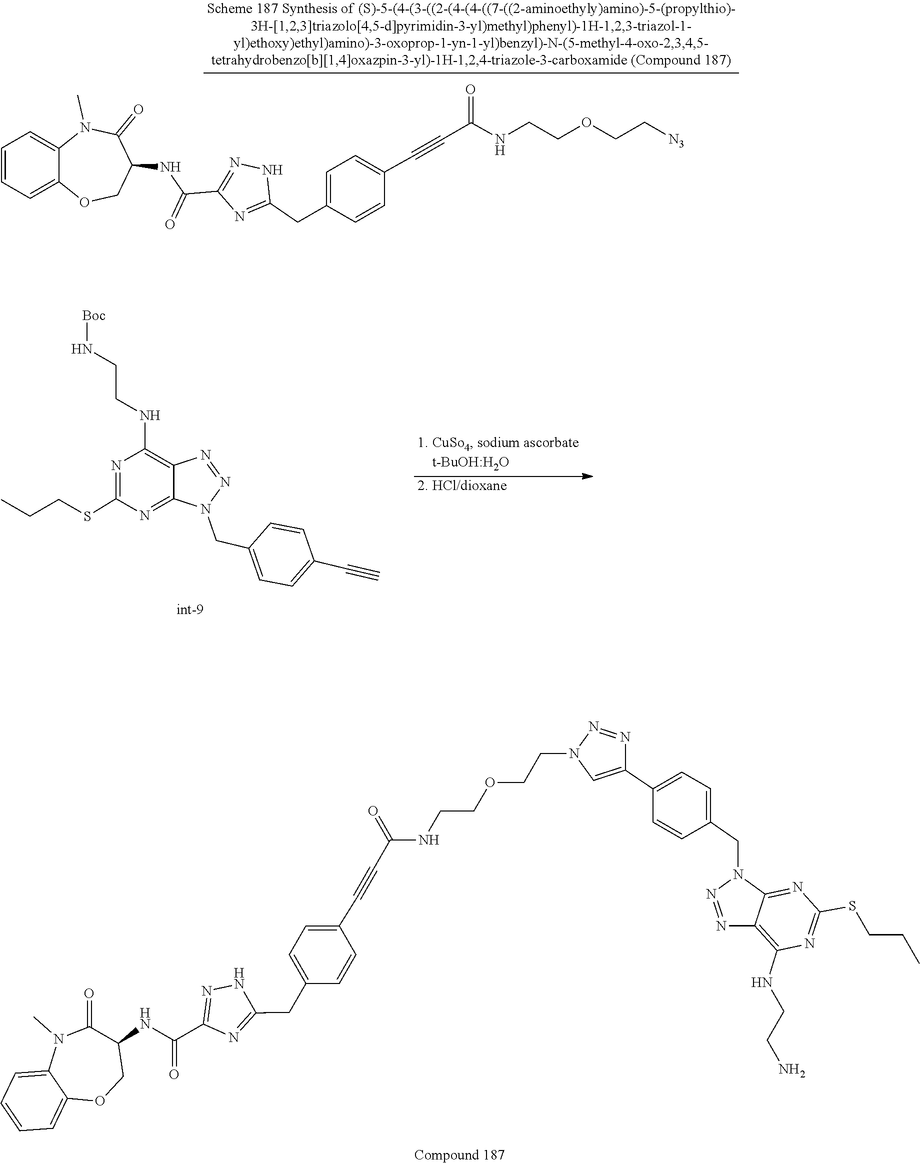 [SMILES: CN1C(=O)[C@@H](NC(=O)c2n[nH]c(Cc3ccc(C#CC(=O)NCCOCCN=[N+]=[N-])cc3)n2)COc2ccccc21 ; C#Cc1ccc(Cn2nnc3c(NCCNC(=O)OC(C)(C)C)nc(SCCC)nc32)cc1 ; CCCSc1nc(NCCN)c2nnn(Cc3ccc(-c4cn(CCOCCNC(=O)C#Cc5ccc(Cc6nc(C(=O)N[C@H]7COc8ccccc8N(C)C7=O)n[nH]6)cc5)nn4)cc3)c2n1]

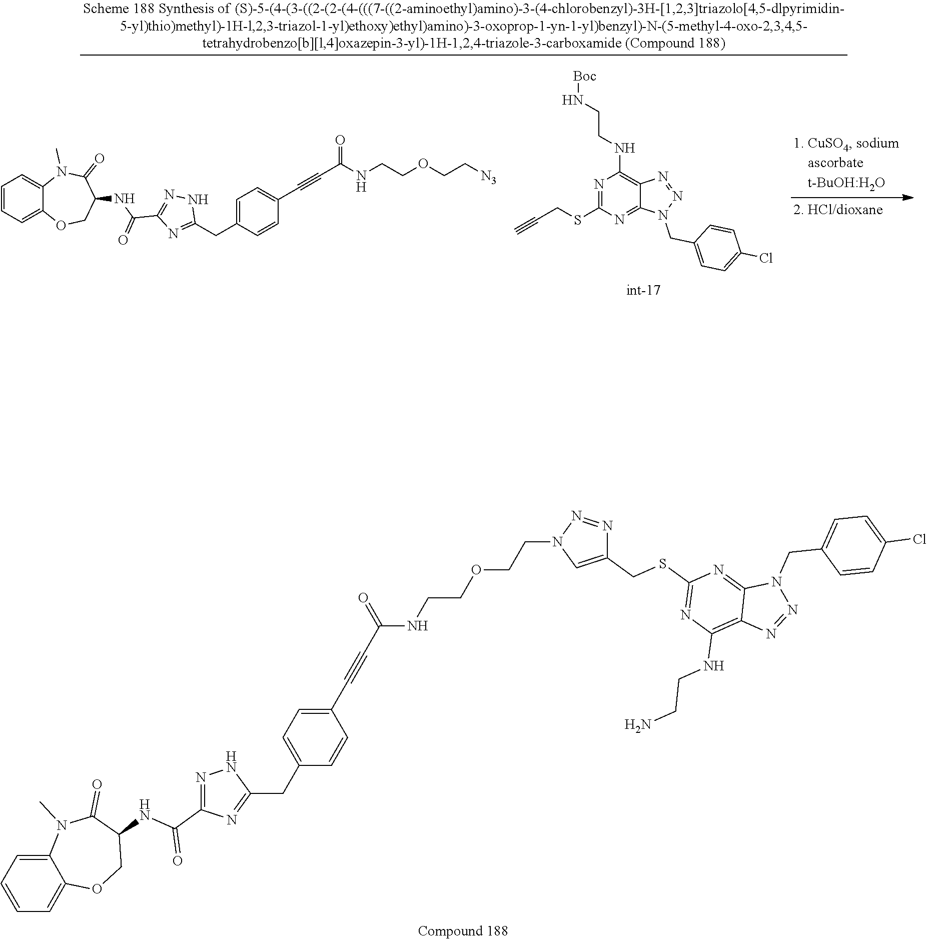 [SMILES: CN1C(=O)[C@@H](NC(=O)c2n[nH]c(Cc3ccc(C#CC(=O)NCCOCCN=[N+]=[N-])cc3)n2)COc2ccccc21 ; C#CCSc1nc(NCCNC(=O)OC(C)(C)C)c2nnn(Cc3ccc(Cl)cc3)c2n1 ; CN1C(=O)[C@@H](NC(=O)c2n[nH]c(Cc3ccc(C#CC(=O)NCCOCCn4cc(CSc5nc(NCCN)c6nnn(Cc7ccc(Cl)cc7)c6n5)nn4)cc3)n2)COc2ccccc21]

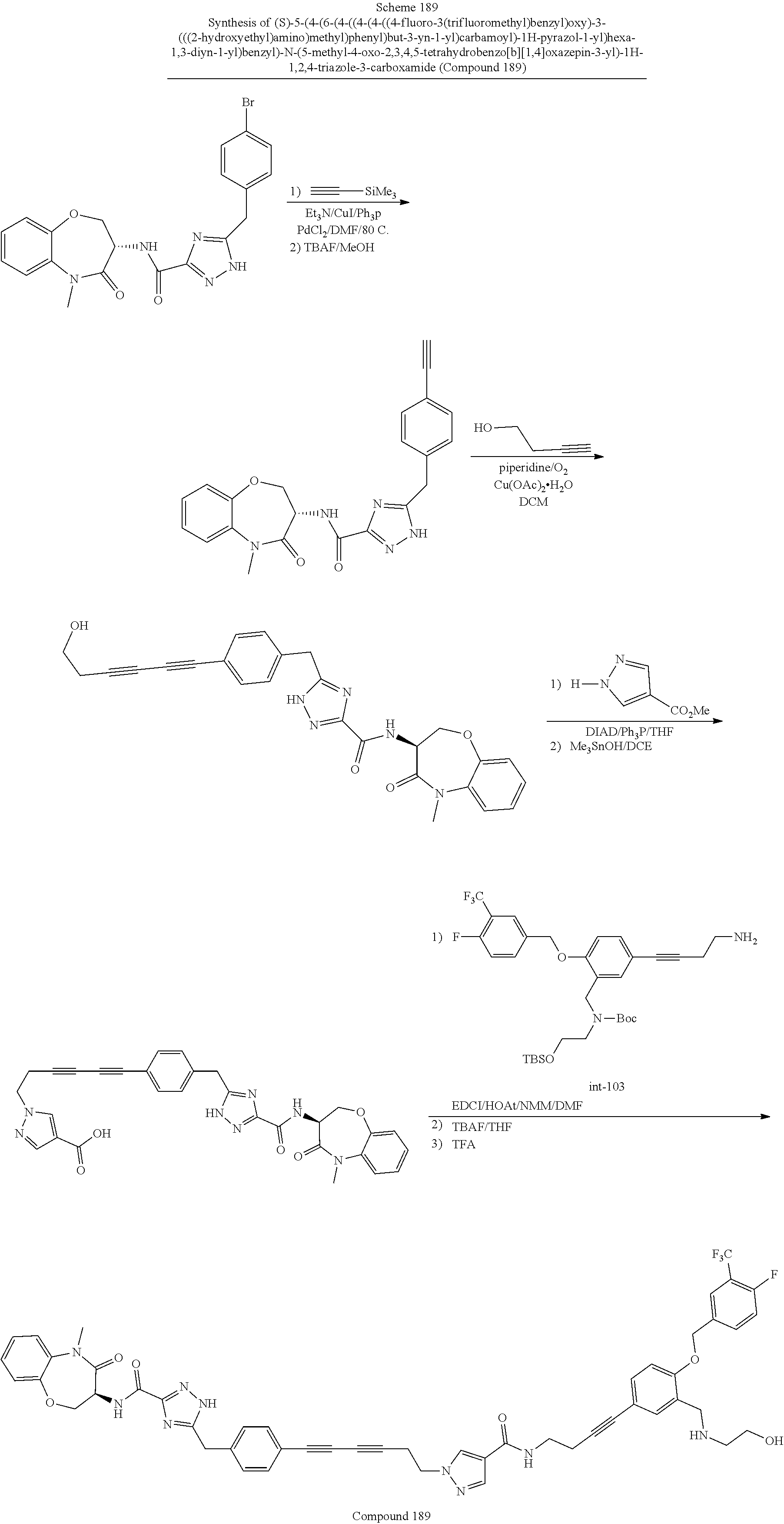

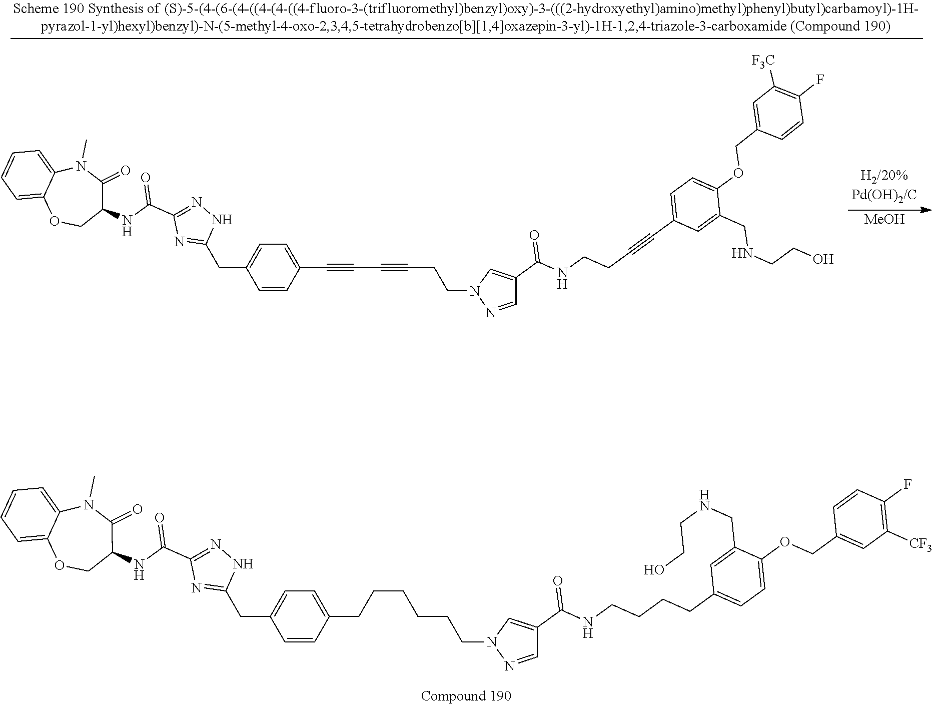 [SMILES: "MeOH" ; CN1C(=O)[C@@H](NC(=O)c2n[nH]c(Cc3ccc(C#CC#CCCn4cc(C(=O)NCCC#Cc5ccc(OCc6ccc(F)c(C(F)(F)F)c6)c(CNCCO)c5)cn4)cc3)n2)COc2ccccc21 ; CN1C(=O)[C@@H](NC(=O)c2n[nH]c(Cc3ccc(CCCCCCn4cc(C(=O)NCCCCc5ccc(OCc6ccc(F)c(C(F)(F)F)c6)c(CNCCO)c5)cn4)cc3)n2)COc2ccccc21]

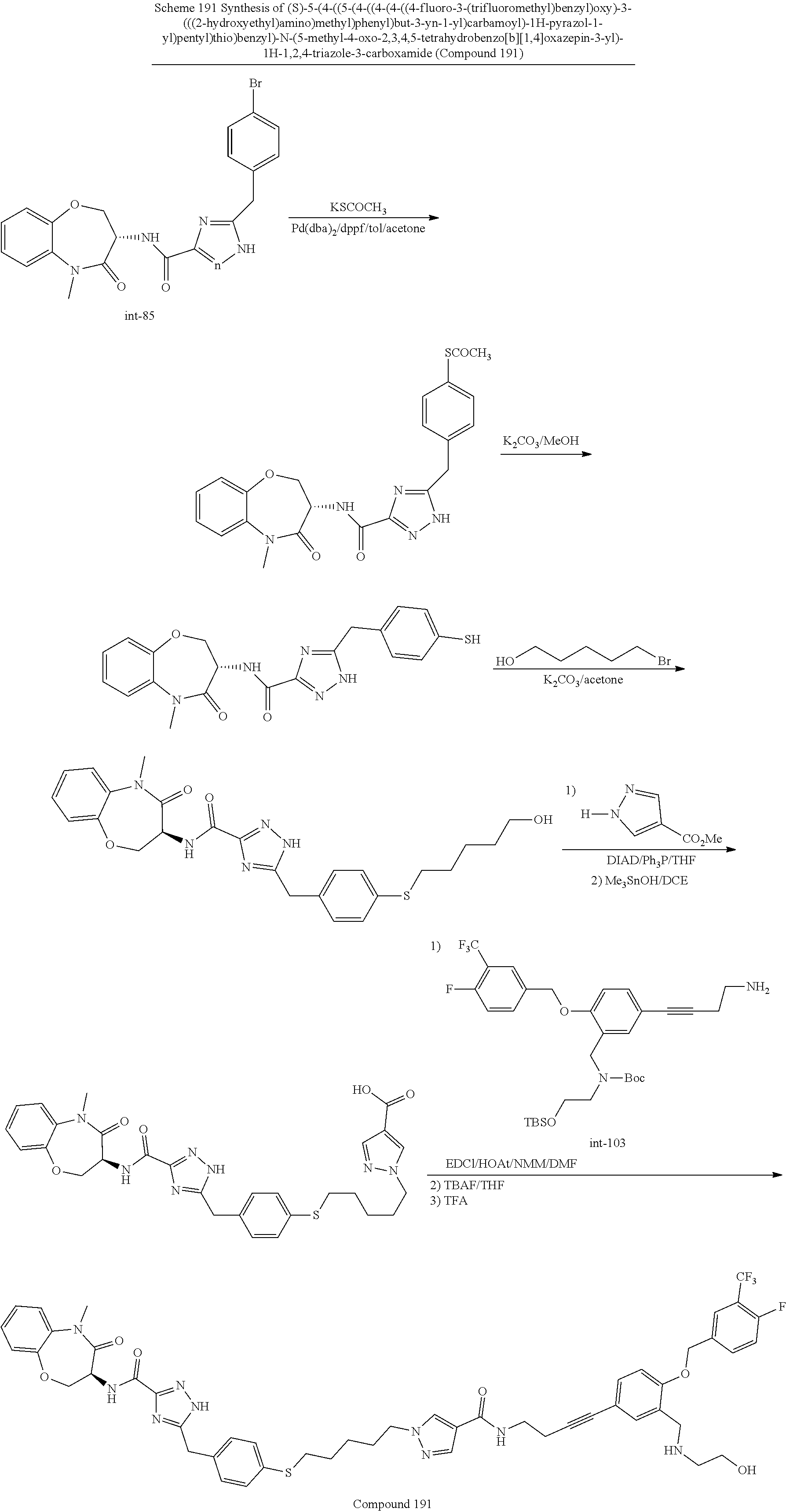

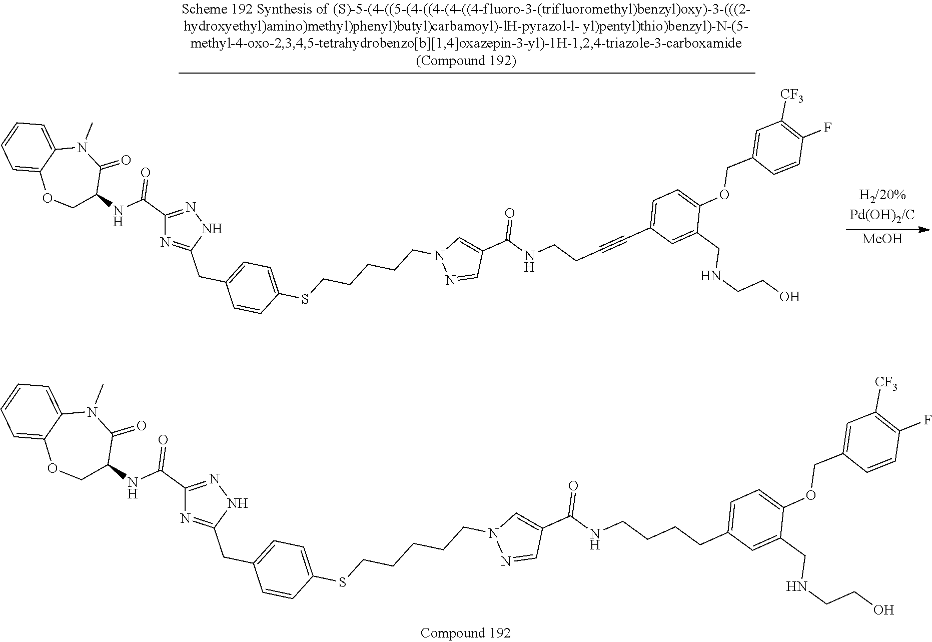 [SMILES: "MeOH" ; CN1C(=O)[C@@H](NC(=O)c2n[nH]c(Cc3ccc(SCCCCCn4cc(C(=O)NCCC#Cc5ccc(OCc6ccc(F)c(C(F)(F)F)c6)c(CNCCO)c5)cn4)cc3)n2)COc2ccccc21 ; CN1C(=O)[C@@H](NC(=O)c2n[nH]c(Cc3ccc(SCCCCCn4cc(C(=O)NCCCCc5ccc(OCc6ccc(F)c(C(F)(F)F)c6)c(CNCCO)c5)cn4)cc3)n2)COc2ccccc21]

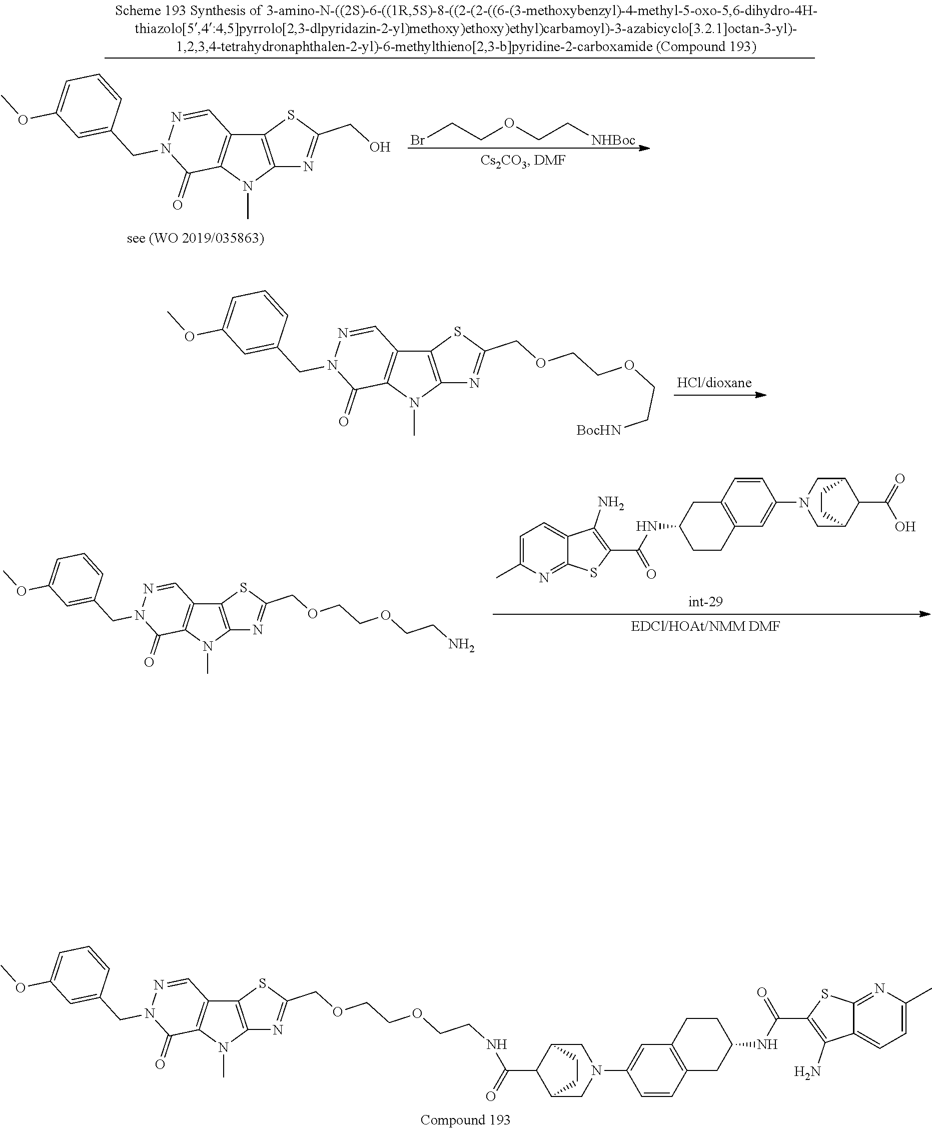

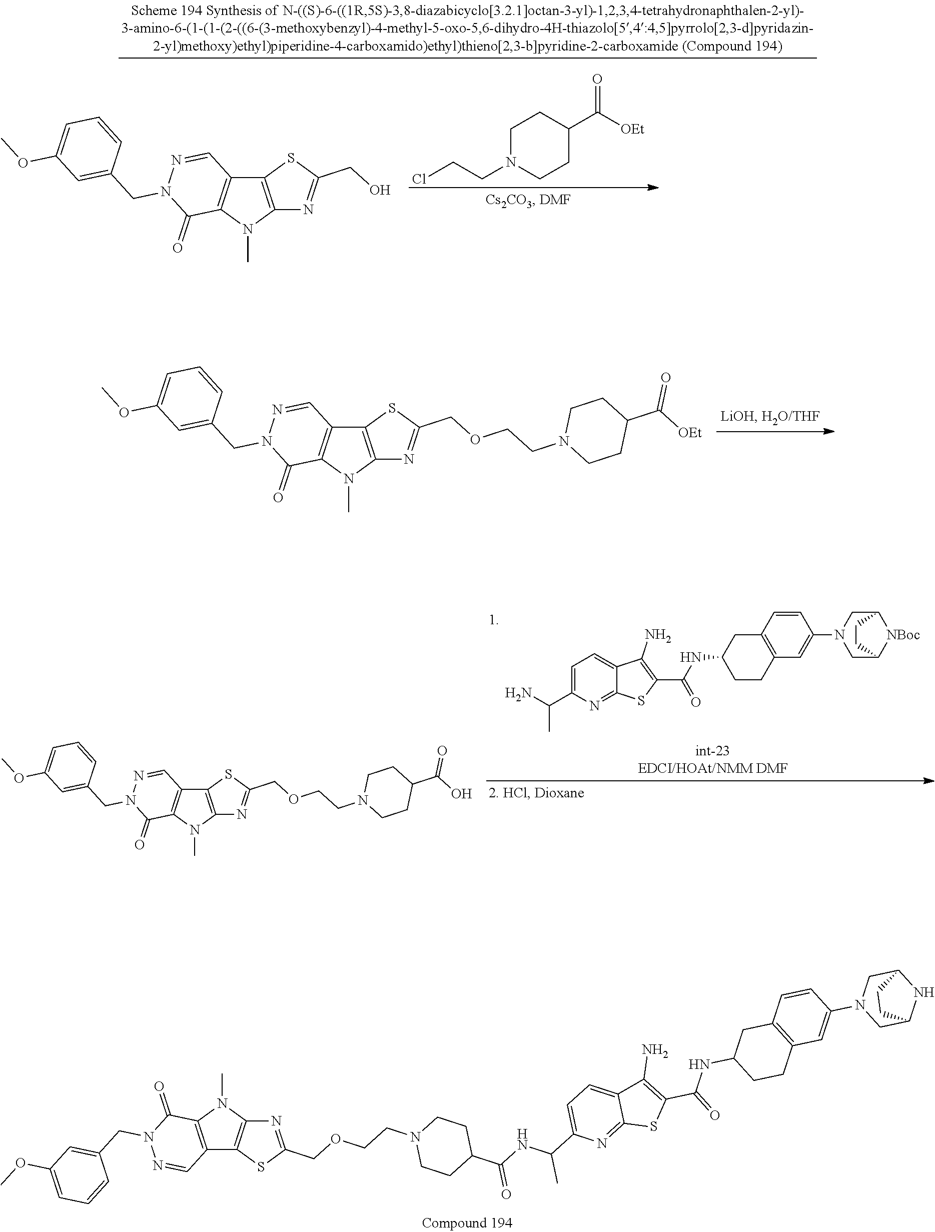 [SMILES: COc1cccc(Cn2ncc3c4sc(CO)nc4n(C)c3c2=O)c1 ; CCOC(=O)C1CCN(CCCl)CC1 ; CCOC(=O)C1CCN(CCOCc2nc3c(s2)c2cnn(Cc4cccc(OC)c4)c(=O)c2n3C)CC1 ; CC(N)c1ccc2c(N)c(C(=O)N[C@H]3CCc4cc(N5C[C@H]6CC[C@@H](C5)N6C(=O)OC(C)(C)C)ccc4C3)sc2n1 ; COc1cccc(Cn2ncc3c4sc(COCCN5CCC(C(=O)O)CC5)nc4n(C)c3c2=O)c1 ; COc1cccc(Cn2ncc3c4sc(COCCN5CCC(C(=O)NC(C)c6ccc7c(N)c(C(=O)NC8CCc9cc(N%10C[C@H]%11CC[C@@H](C%10)N%11)ccc9C8)sc7n6)CC5)nc4n(C)c3c2=O)c1]

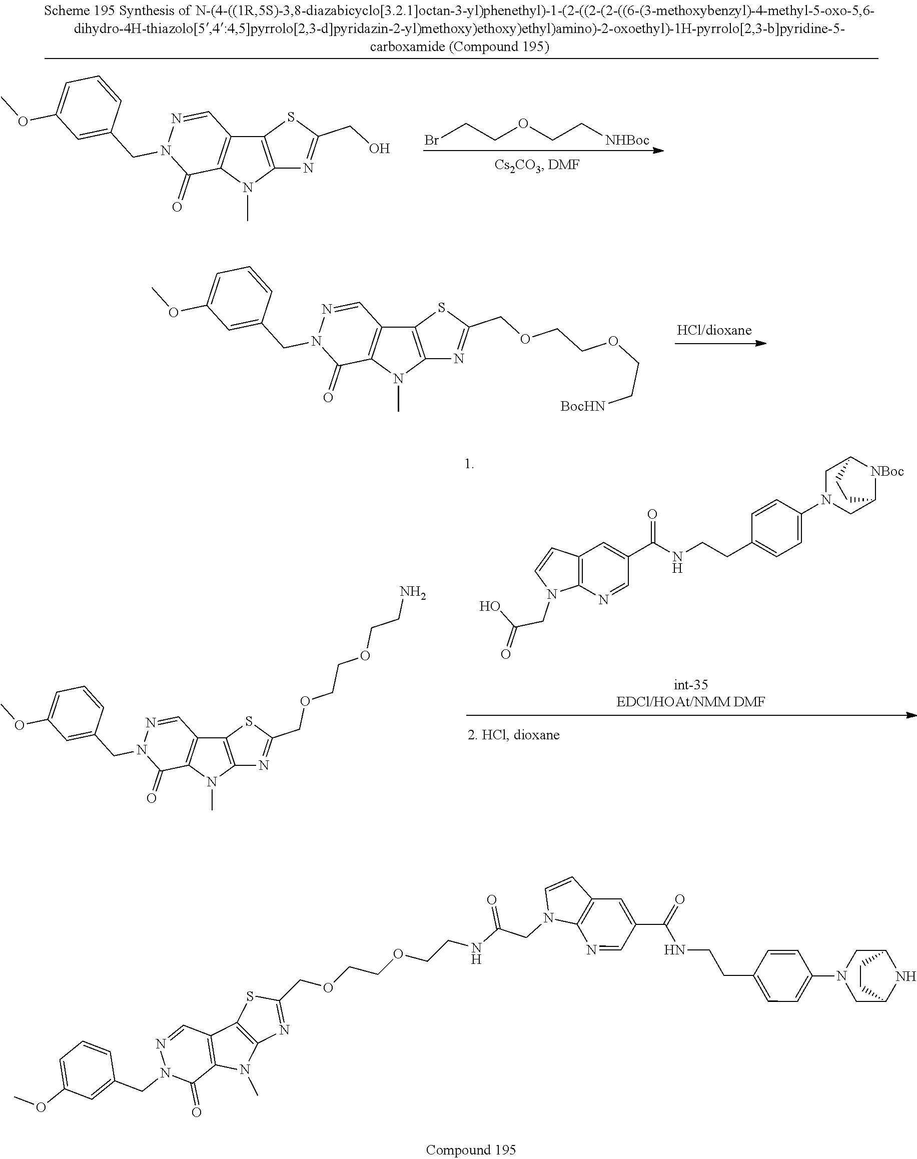 [SMILES: COc1cccc(Cn2ncc3c4sc(CO)nc4n(C)c3c2=O)c1 ; CC(C)(C)OC(=O)NCCOCCBr ; COc1cccc(Cn2ncc3c4sc(COCCOCCNC(=O)OC(C)(C)C)nc4n(C)c3c2=O)c1 ; COc1cccc(Cn2ncc3c4sc(COCCOCCN)nc4n(C)c3c2=O)c1 ; CC(C)(C)OC(=O)N1[C@@H]2CC[C@H]1CN(c1ccc(CCNC(=O)c3cnc4c(ccn4CC(=O)O)c3)cc1)C2 ; COc1cccc(Cn2ncc3c4sc(COCCOCCNC(=O)Cn5ccc6cc(C(=O)NCCc7ccc(N8C[C@H]9CC[C@@H](C8)N9)cc7)cnc65)nc4n(C)c3c2=O)c1]

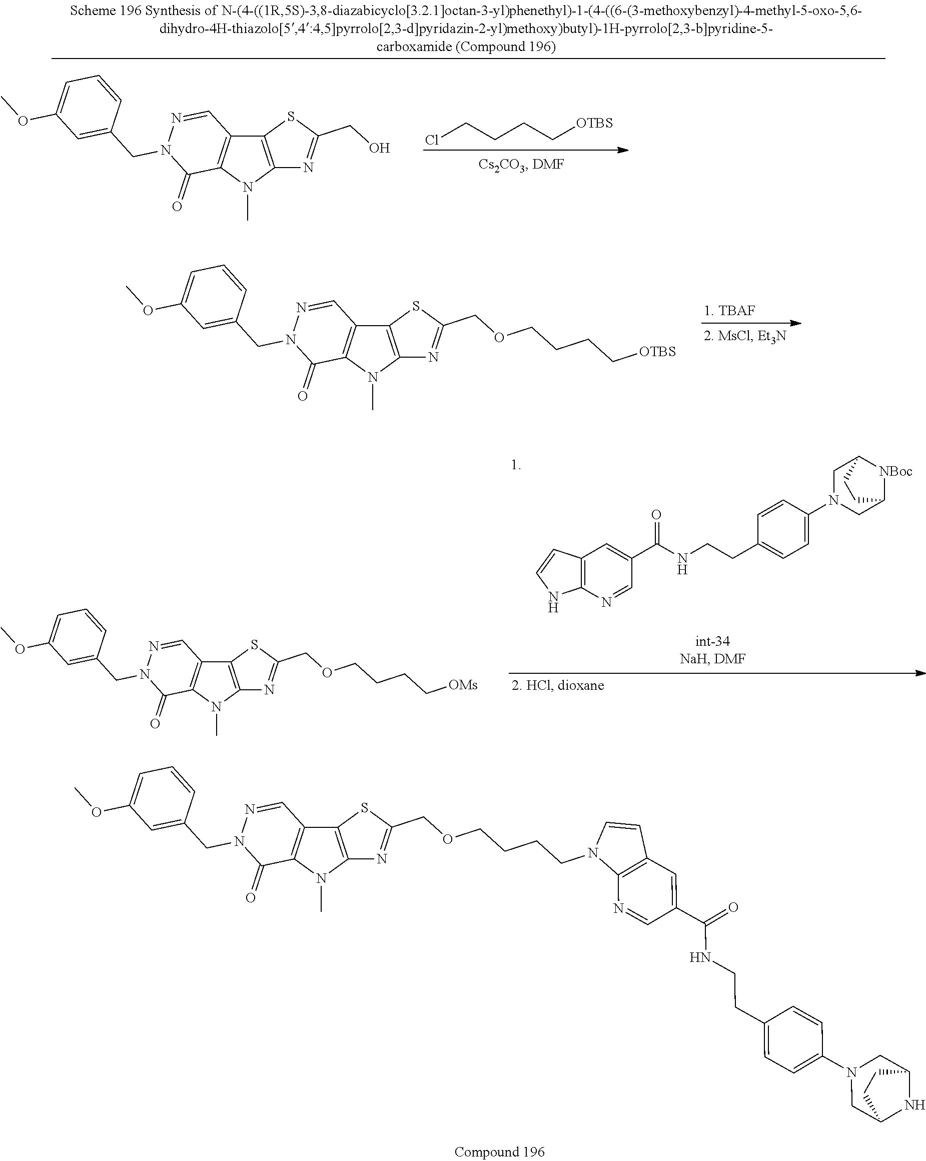

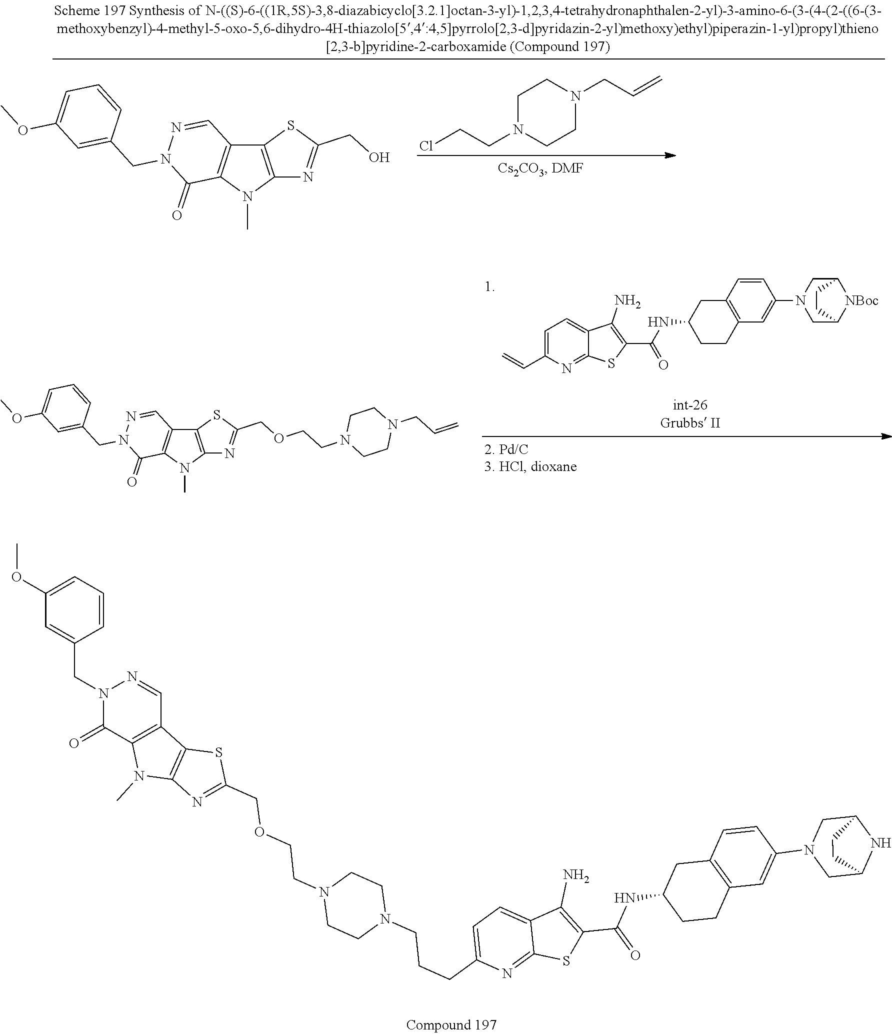 [SMILES: COc1cccc(Cn2ncc3c4sc(CO)nc4n(C)c3c2=O)c1 ; C=CCN1CCN(CCCl)CC1 ; C=Cc1ccc2c(N)c(C(=O)N[C@H]3CCc4cc(N5C[C@H]6CC[C@@H](C5)N6C(=O)OC(C)(C)C)ccc4C3)sc2n1 ; C=CCN1CCN(CCOCc2nc3c(s2)c2cnn(Cc4cccc(OC)c4)c(=O)c2n3C)CC1 ; COc1cccc(Cn2ncc3c4sc(COCCN5CCN(CCCc6ccc7c(N)c(C(=O)N[C@H]8CCc9cc(N%10C[C@H]%11CC[C@@H](C%10)N%11)ccc9C8)sc7n6)CC5)nc4n(C)c3c2=O)c1]

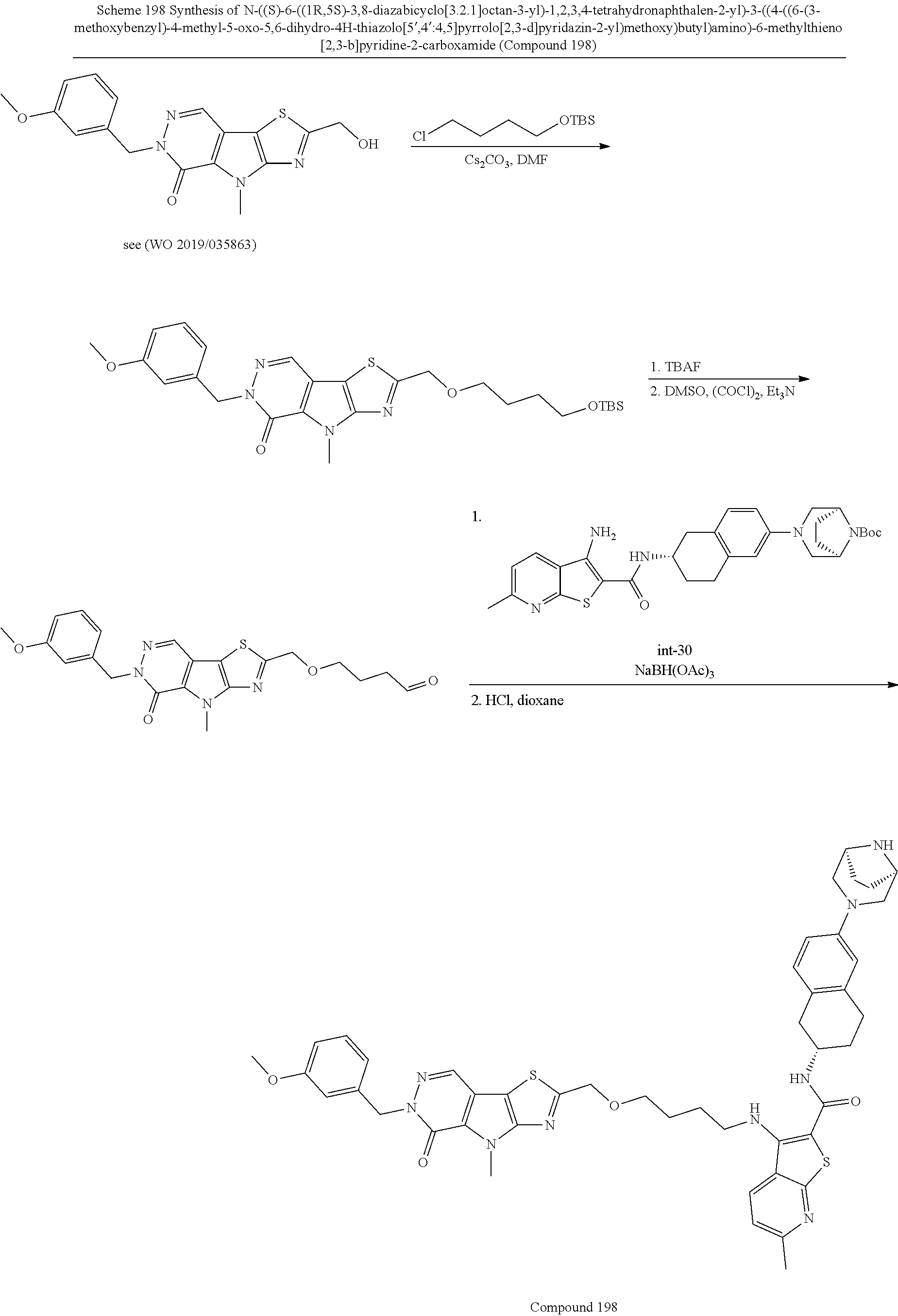 [SMILES: COc1cccc(Cn2ncc3c4sc(CO)nc4n(C)c3c2=O)c1 ; CC(C)(C)[Si](C)(C)OCCCCCl ; COc1cccc(Cn2ncc3c4sc(COCCCCO[Si](C)(C)C(C)(C)C)nc4n(C)c3c2=O)c1 ; Cc1ccc2c(N)c(C(=O)N[C@H]3CCc4cc(N5C[C@H]6CC[C@@H](C5)N6C(=O)OC(C)(C)C)ccc4C3)sc2n1 ; COc1cccc(Cn2ncc3c4sc(COCCCC=O)nc4n(C)c3c2=O)c1 ; COc1cccc(Cn2ncc3c4sc(COCCCCNc5c(C(=O)N[C@H]6CCc7cc(N8C[C@H]9CC[C@@H](C8)N9)ccc7C6)sc6nc(C)ccc56)nc4n(C)c3c2=O)c1]

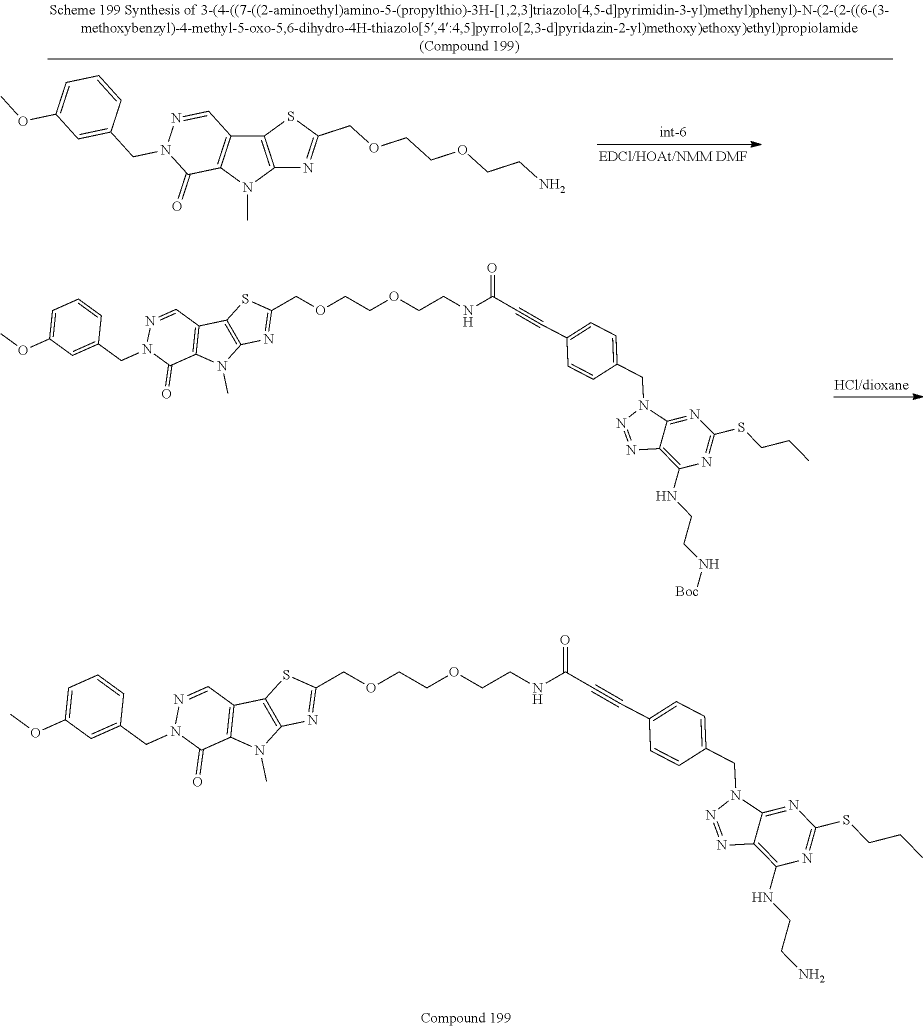

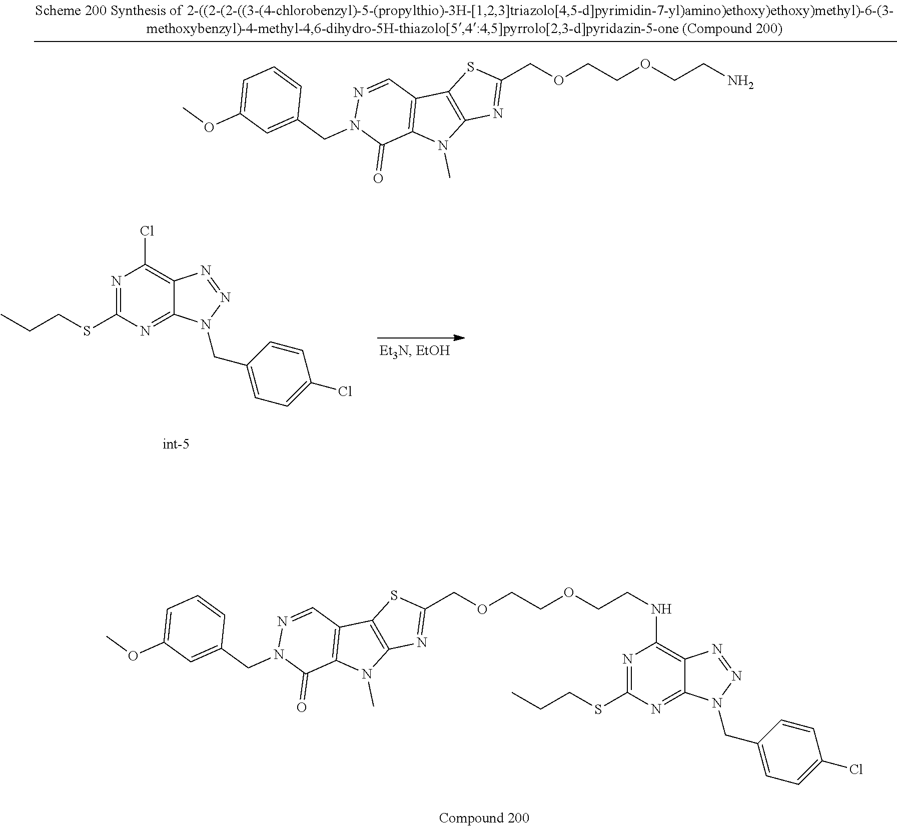 [SMILES: COc1cccc(Cn2ncc3c4sc(COCCOCCN)nc4n(C)c3c2=O)c1 ; CCCSc1nc(Cl)c2nnn(Cc3ccc(Cl)cc3)c2n1 ; CCCSc1nc(NCCOCCOCc2nc3c(s2)c2cnn(Cc4cccc(OC)c4)c(=O)c2n3C)c2nnn(Cc3ccc(Cl)cc3)c2n1]

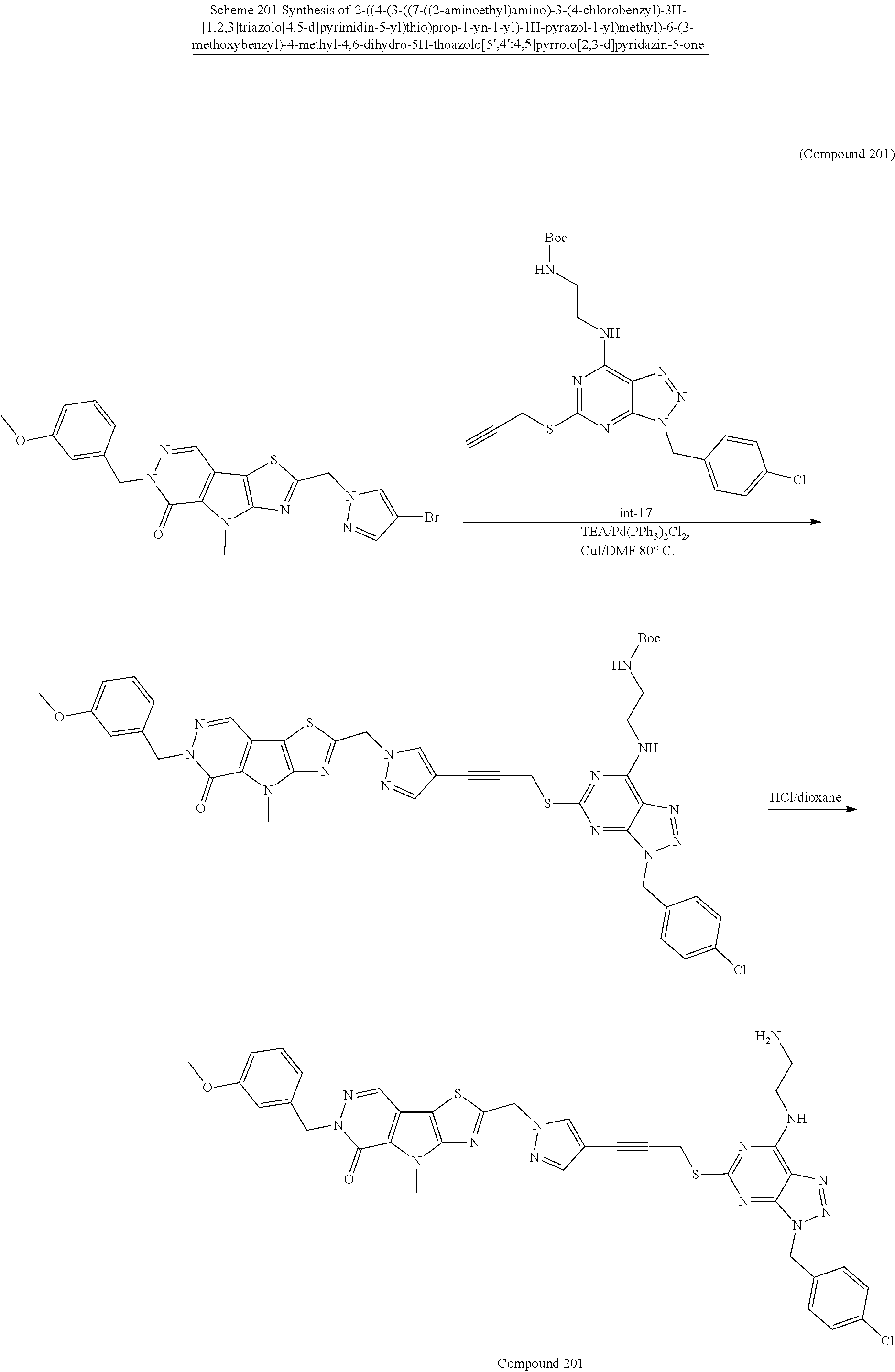 [SMILES: COc1cccc(Cn2ncc3c4sc(Cn5cc(Br)cn5)nc4n(C)c3c2=O)c1 ; C#CCSc1nc(NCCNC(=O)OC(C)(C)C)c2nnn(Cc3ccc(Cl)cc3)c2n1 ; COc1cccc(Cn2ncc3c4sc(Cn5cc(C#CCSc6nc(NCCNC(=O)OC(C)(C)C)c7nnn(Cc8ccc(Cl)cc8)c7n6)cn5)nc4n(C)c3c2=O)c1 ; COc1cccc(Cn2ncc3c4sc(Cn5cc(C#CCSc6nc(NCCN)c7nnn(Cc8ccc(Cl)cc8)c7n6)cn5)nc4n(C)c3c2=O)c1]

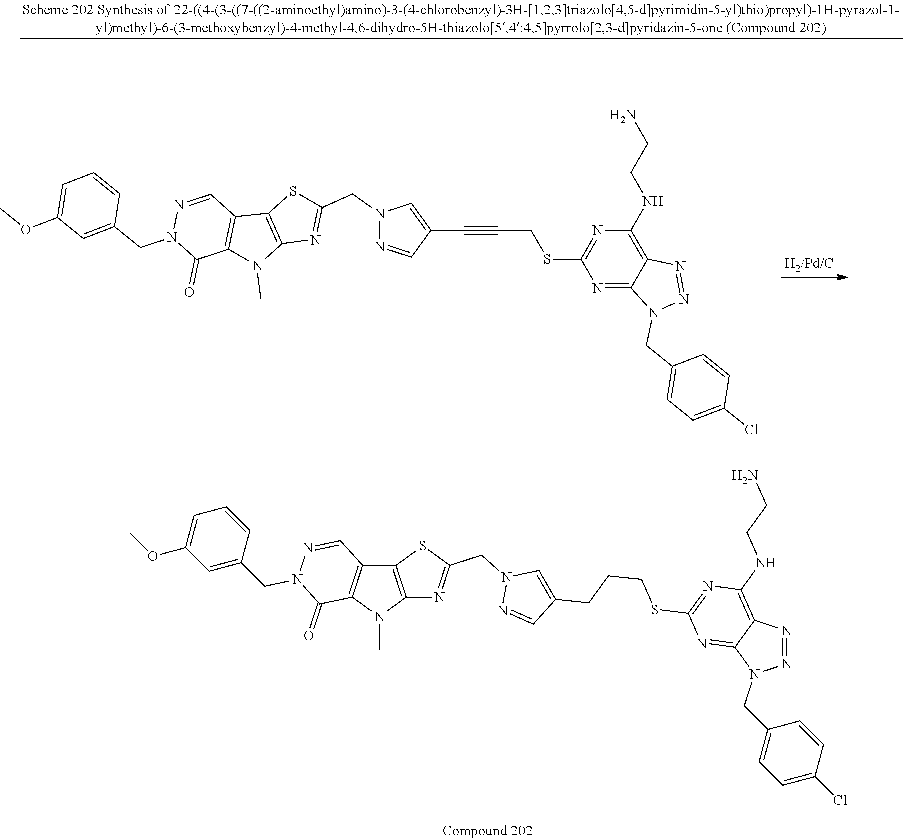

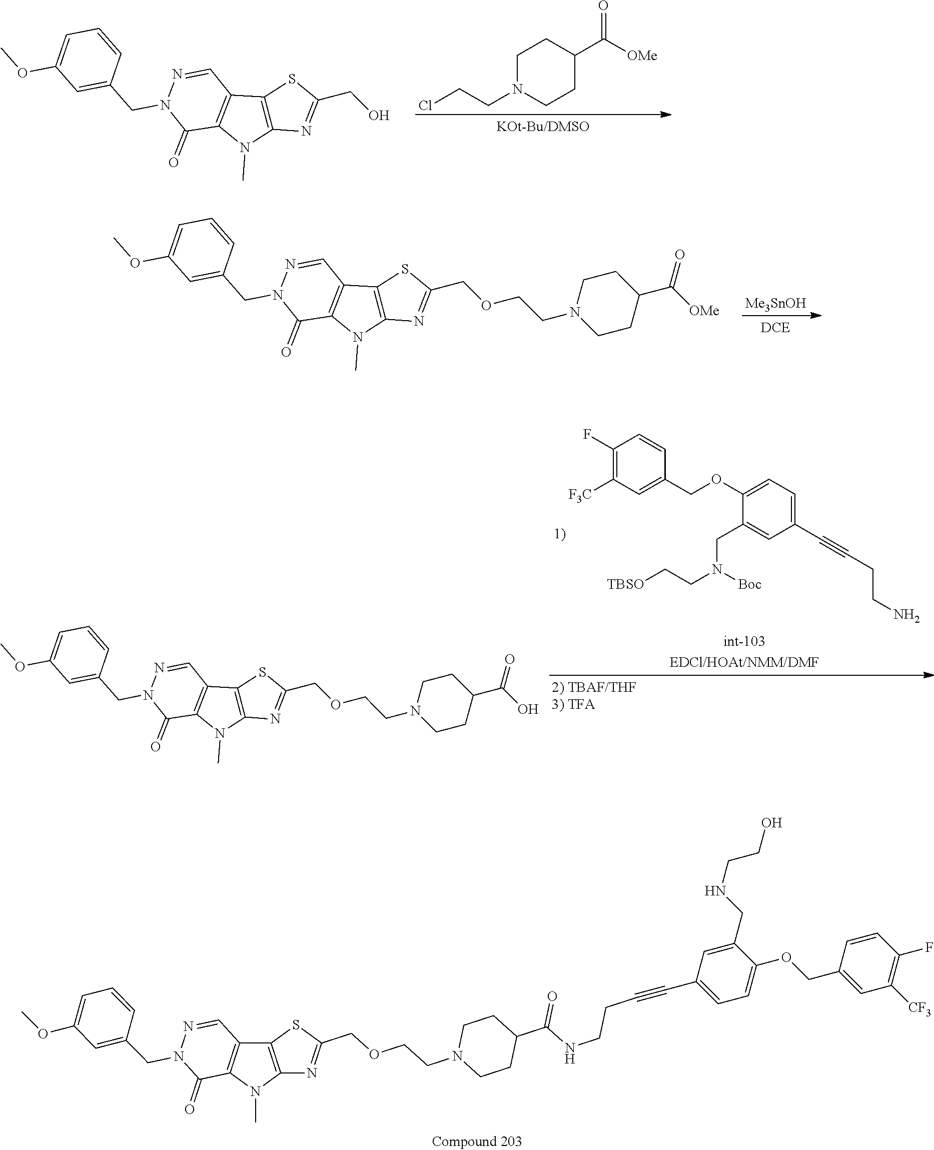

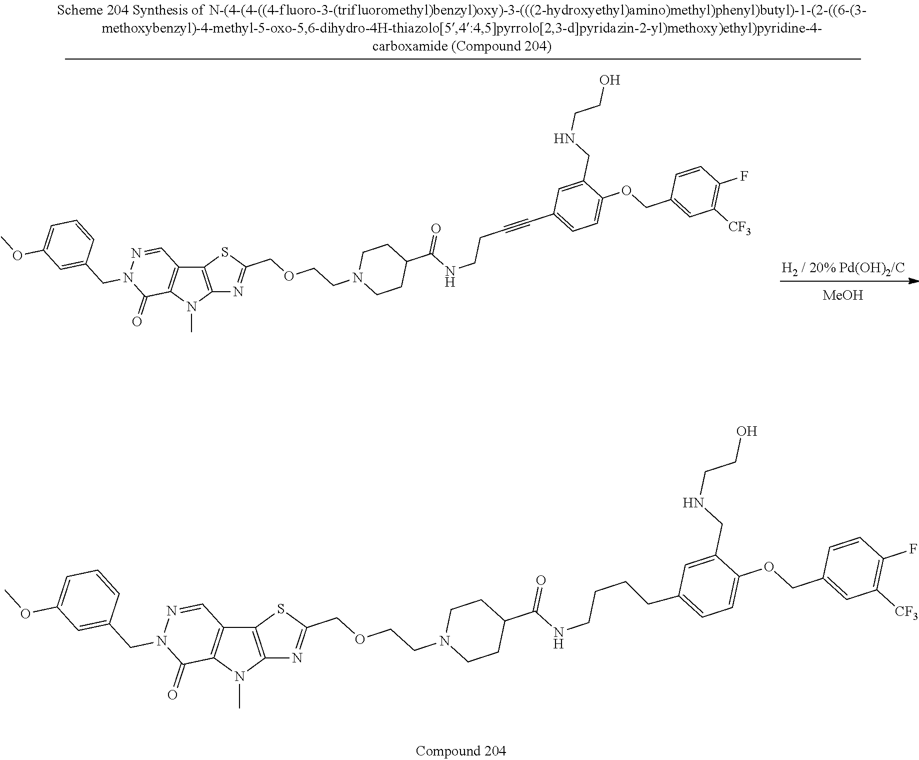 [SMILES: "MeOH" ; COc1cccc(Cn2ncc3c4sc(COCCN5CCC(C(=O)NCCC#Cc6ccc(OCc7ccc(F)c(C(F)(F)F)c7)c(CNCCO)c6)CC5)nc4n(C)c3c2=O)c1 ; COc1cccc(Cn2ncc3c4sc(COCCN5CCC(C(=O)NCCCCc6ccc(OCc7ccc(F)c(C(F)(F)F)c7)c(CNCCO)c6)CC5)nc4n(C)c3c2=O)c1]

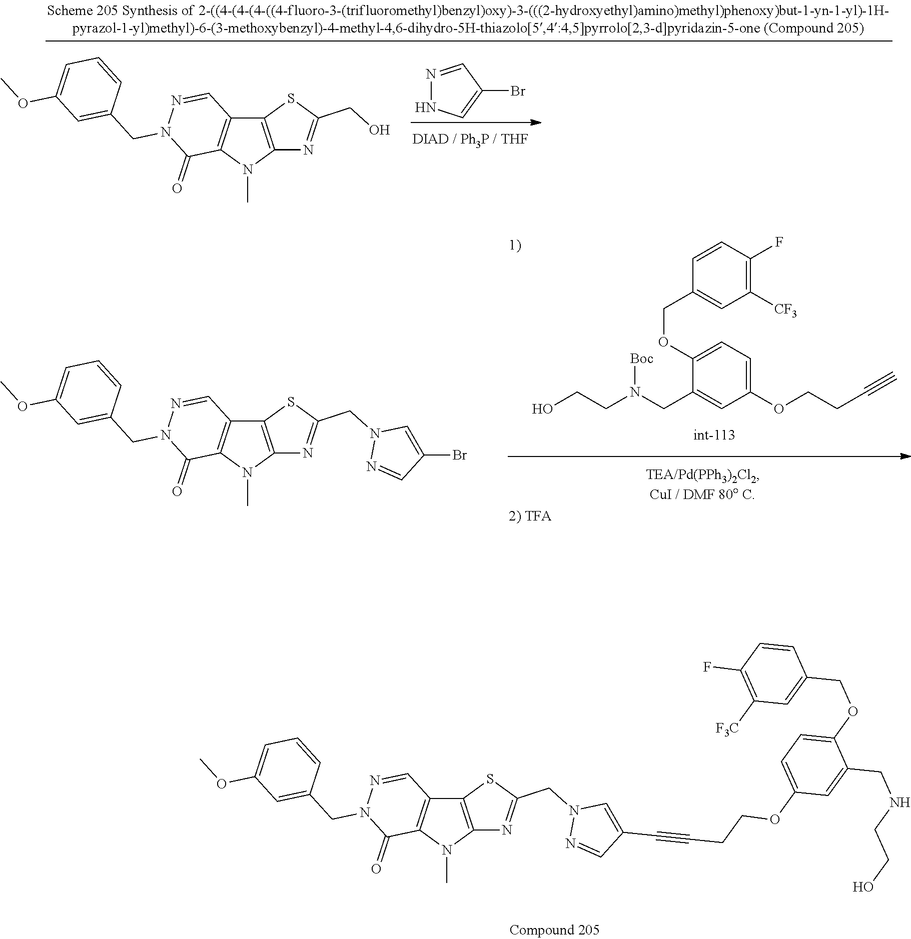 [SMILES: COc1cccc(Cn2ncc3c4sc(CO)nc4n(C)c3c2=O)c1 ; Brc1cn[nH]c1 ; COc1cccc(Cn2ncc3c4sc(Cn5cc(Br)cn5)nc4n(C)c3c2=O)c1 ; C#CCCOc1ccc(OCc2ccc(F)c(C(F)(F)F)c2)c(CN(CCO)C(=O)OC(C)(C)C)c1 ; COc1cccc(Cn2ncc3c4sc(Cn5cc(C#CCCOc6ccc(OCc7ccc(F)c(C(F)(F)F)c7)c(CNCCO)c6)cn5)nc4n(C)c3c2=O)c1]

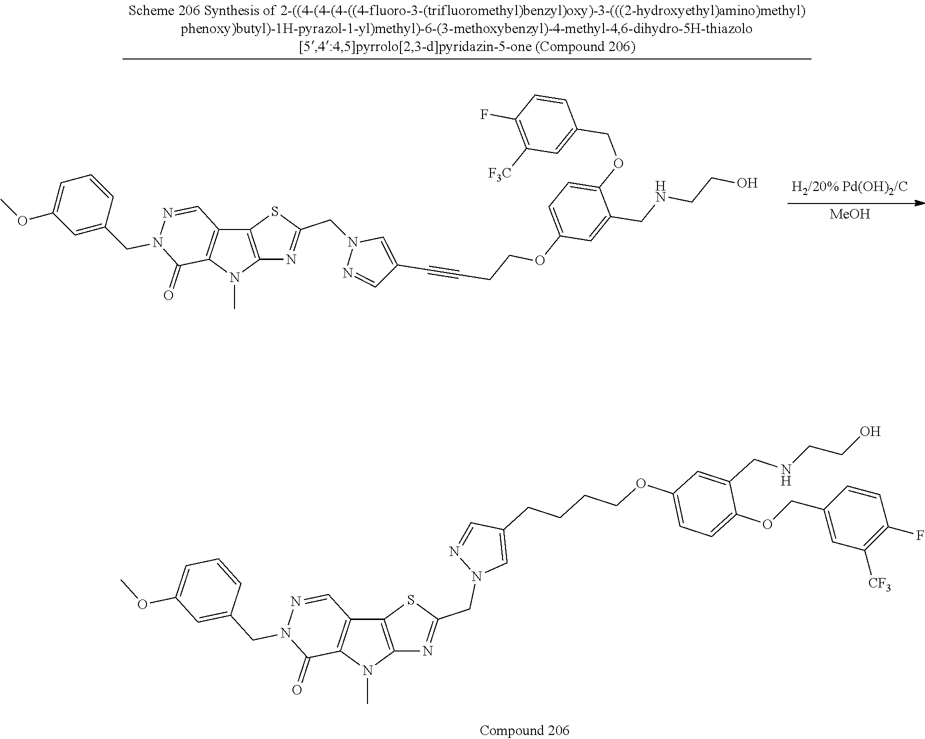 [SMILES: "MeOH" ; COc1cccc(Cn2ncc3c4sc(Cn5cc(C#CCCOc6ccc(OCc7ccc(F)c(C(F)(F)F)c7)c(CNCCO)c6)cn5)nc4n(C)c3c2=O)c1 ; COc1cccc(Cn2ncc3c4sc(Cn5cc(CCCCOc6ccc(OCc7ccc(F)c(C(F)(F)F)c7)c(CNCCO)c6)cn5)nc4n(C)c3c2=O)c1]

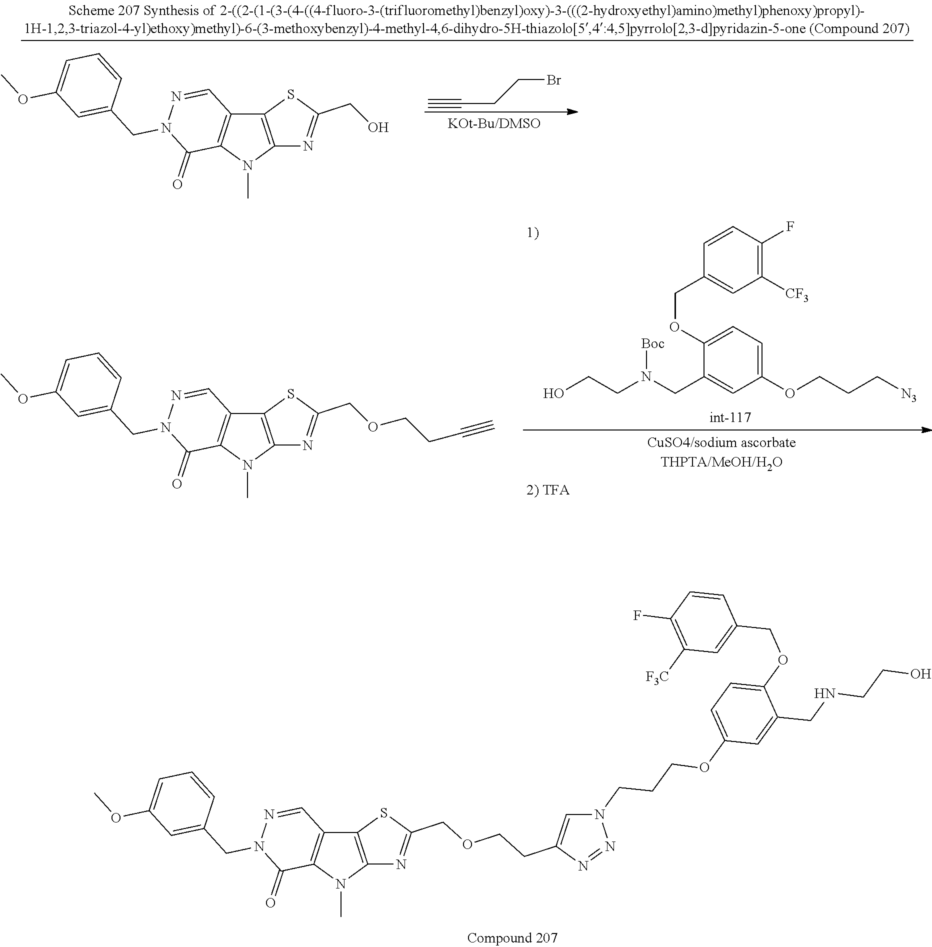

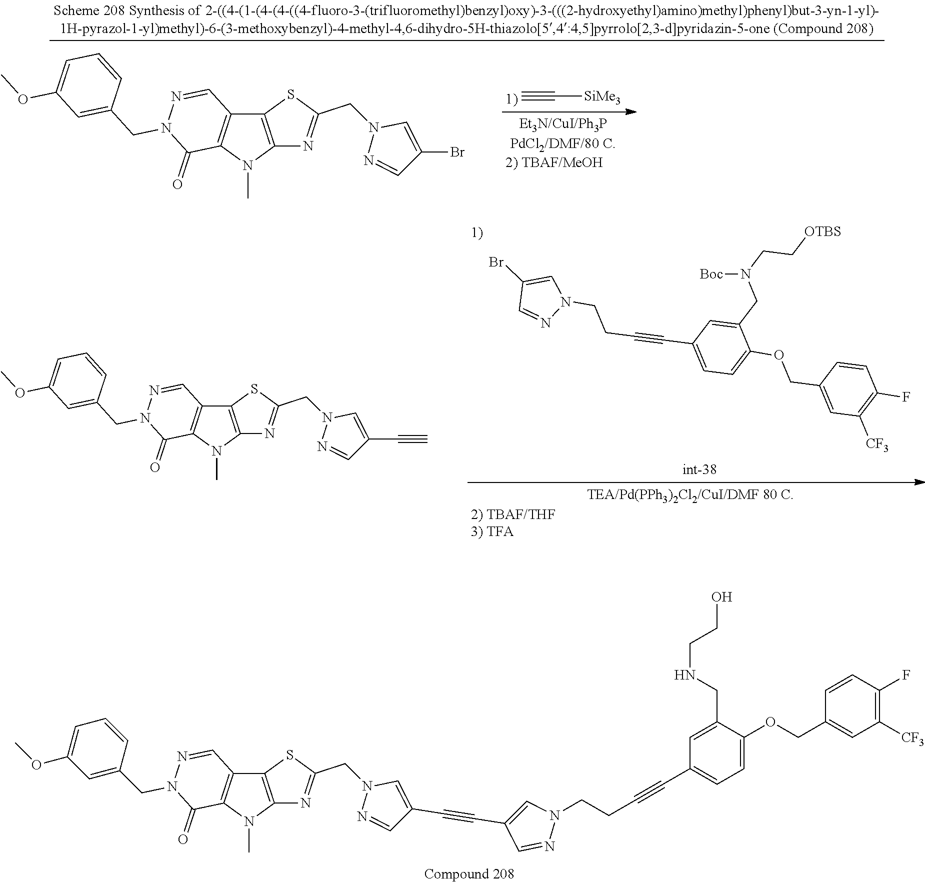

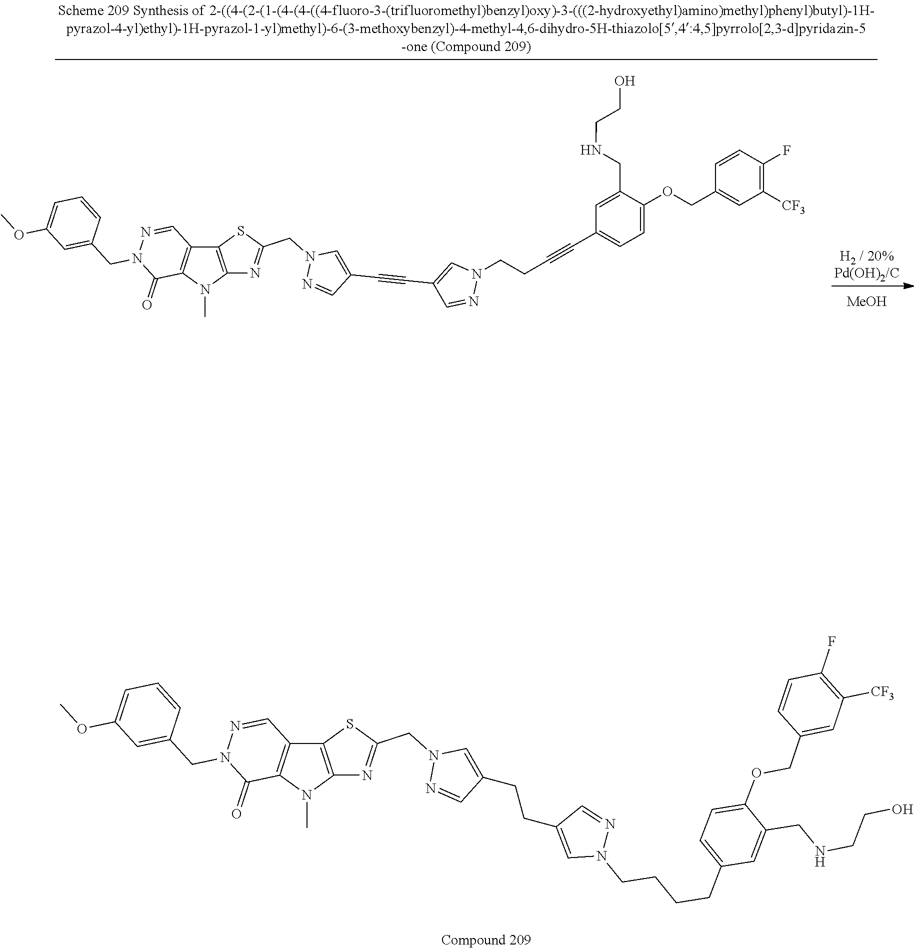 [SMILES: "MeOH" ; COc1cccc(Cn2ncc3c4sc(Cn5cc(C#Cc6cnn(CCC#Cc7ccc(OCc8ccc(F)c(C(F)(F)F)c8)c(CNCCO)c7)c6)cn5)nc4n(C)c3c2=O)c1 ; COc1cccc(Cn2ncc3c4sc(Cn5cc(CCc6cnn(CCCCc7ccc(OCc8ccc(F)c(C(F)(F)F)c8)c(CNCCO)c7)c6)cn5)nc4n(C)c3c2=O)c1]

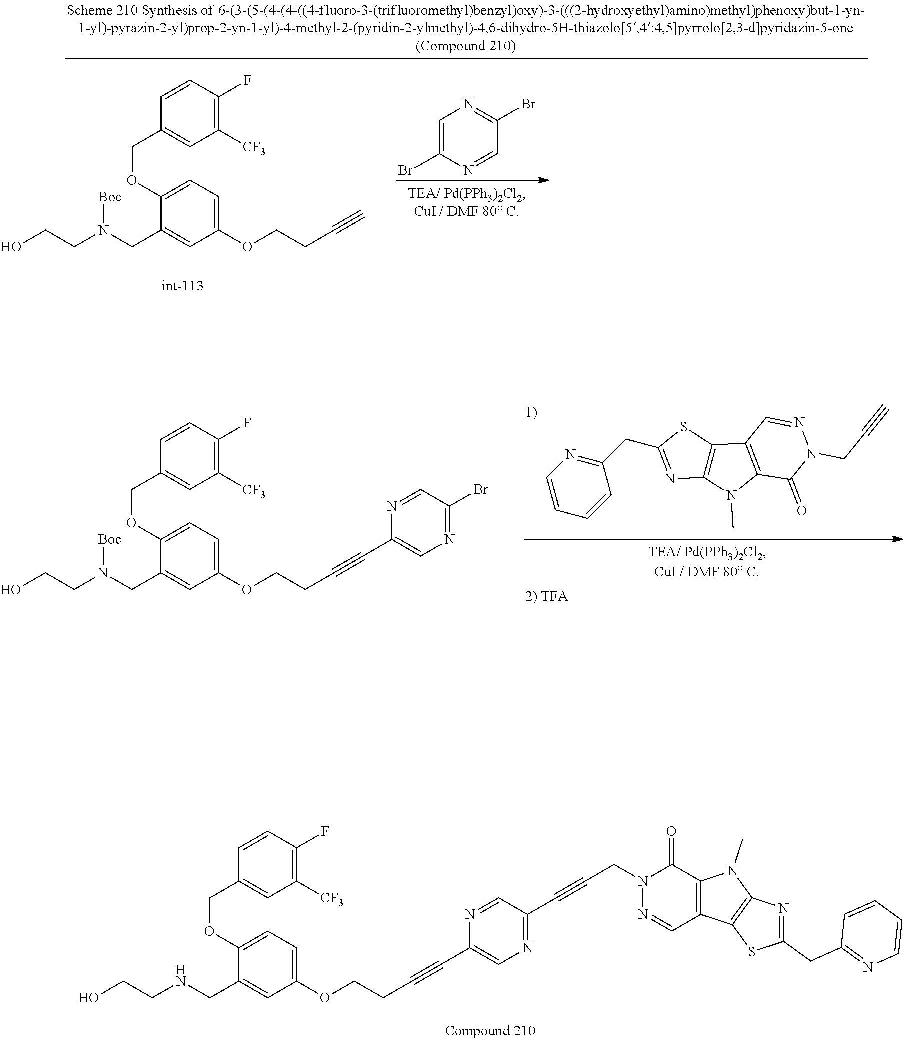 [SMILES: C#CCCOc1ccc(OCc2ccc(F)c(C(F)(F)F)c2)c(CN(CCO)C(=O)OC(C)(C)C)c1 ; Brc1cnc(Br)cn1 ; CC(C)(C)OC(=O)N(CCO)Cc1cc(OCCC#Cc2cnc(Br)cn2)ccc1OCc1ccc(F)c(C(F)(F)F)c1 ; C#CCn1ncc2c3sc(Cc4ccccn4)nc3n(C)c2c1=O ; Cn1c2nc(Cc3ccccn3)sc2c2cnn(CC#Cc3cnc(C#CCCOc4ccc(OCc5ccc(F)c(C(F)(F)F)c5)c(CNCCO)c4)cn3)c(=O)c21]

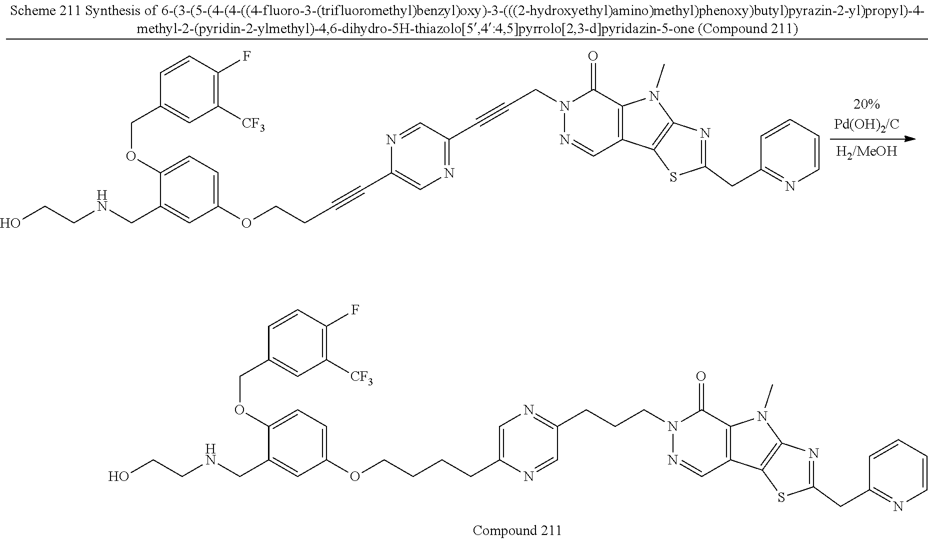 [SMILES: Cn1c2nc(Cc3ccccn3)sc2c2cnn(CC#Cc3cnc(C#CCCOc4ccc(OCc5ccc(F)c(C(F)(F)F)c5)c(CNCCO)c4)cn3)c(=O)c21 ; Cn1c2nc(Cc3ccccn3)sc2c2cnn(CCCc3cnc(CCCCOc4ccc(OCc5ccc(F)c(C(F)(F)F)c5)c(CNCCO)c4)cn3)c(=O)c21]

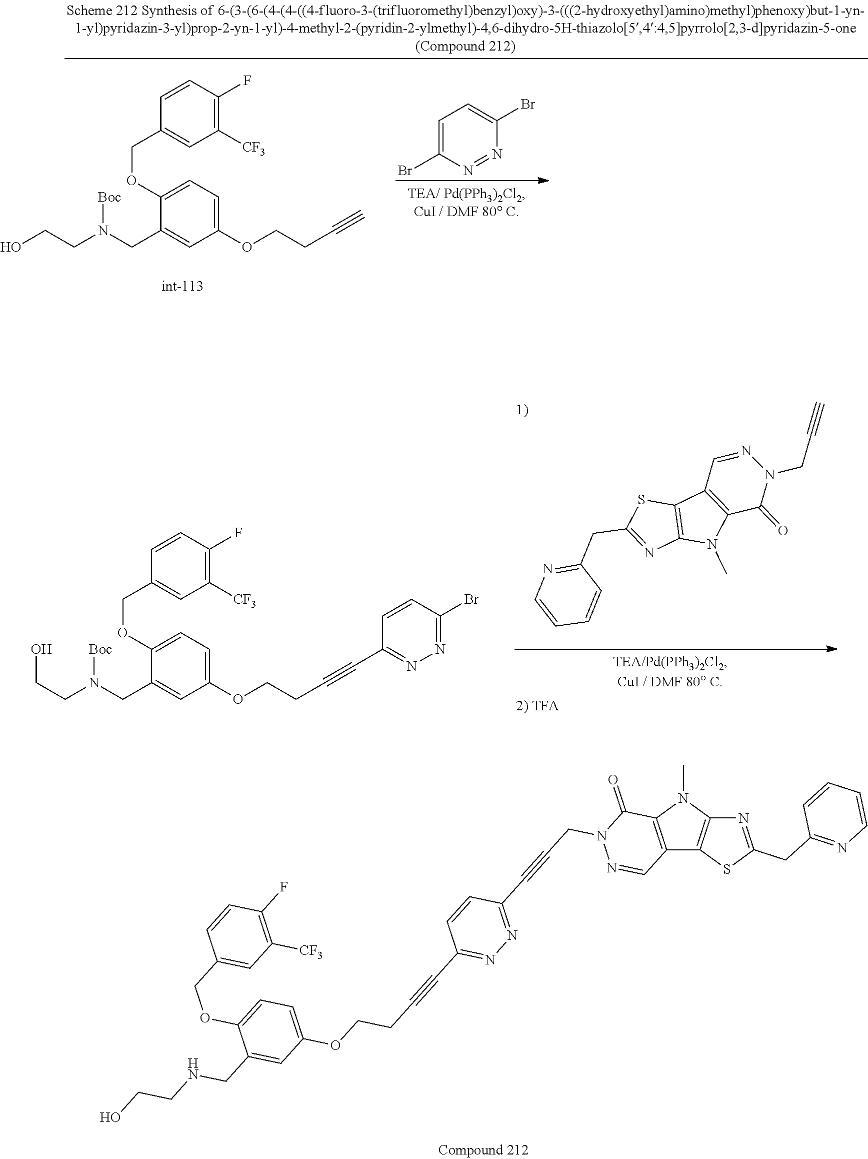 [SMILES: C#CCCOc1ccc(OCc2ccc(F)c(C(F)(F)F)c2)c(CN(CCO)C(=O)OC(C)(C)C)c1 ; Brc1ccc(Br)nn1 ; CC(C)(C)OC(=O)N(CCO)Cc1cc(OCCC#Cc2ccc(Br)nn2)ccc1OCc1ccc(F)c(C(F)(F)F)c1 ; C#CCn1ncc2c3sc(Cc4ccccn4)nc3n(C)c2c1=O ; Cn1c2nc(Cc3ccccn3)sc2c2cnn(CC#Cc3ccc(C#CCCOc4ccc(OCc5ccc(F)c(C(F)(F)F)c5)c(CNCCO)c4)nn3)c(=O)c21]

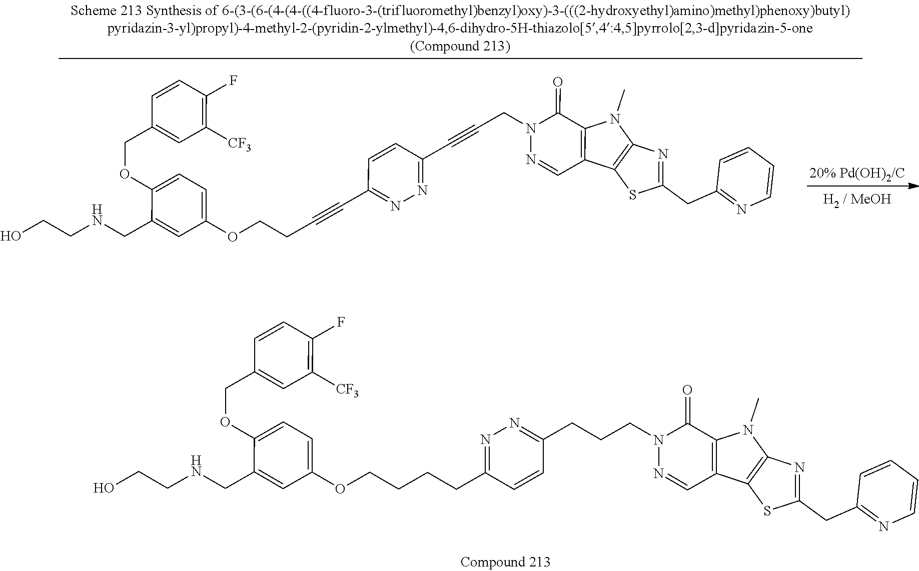 [SMILES: Cn1c2nc(Cc3ccccn3)sc2c2cnn(CC#Cc3ccc(C#CCCOc4ccc(OCc5ccc(F)c(C(F)(F)F)c5)c(CNCCO)c4)nn3)c(=O)c21 ; "H2 / MeOH" ; Cn1c2nc(Cc3ccccn3)sc2c2cnn(CCCc3ccc(CCCCOc4ccc(OCc5ccc(F)c(C(F)(F)F)c5)c(CNCCO)c4)nn3)c(=O)c21]

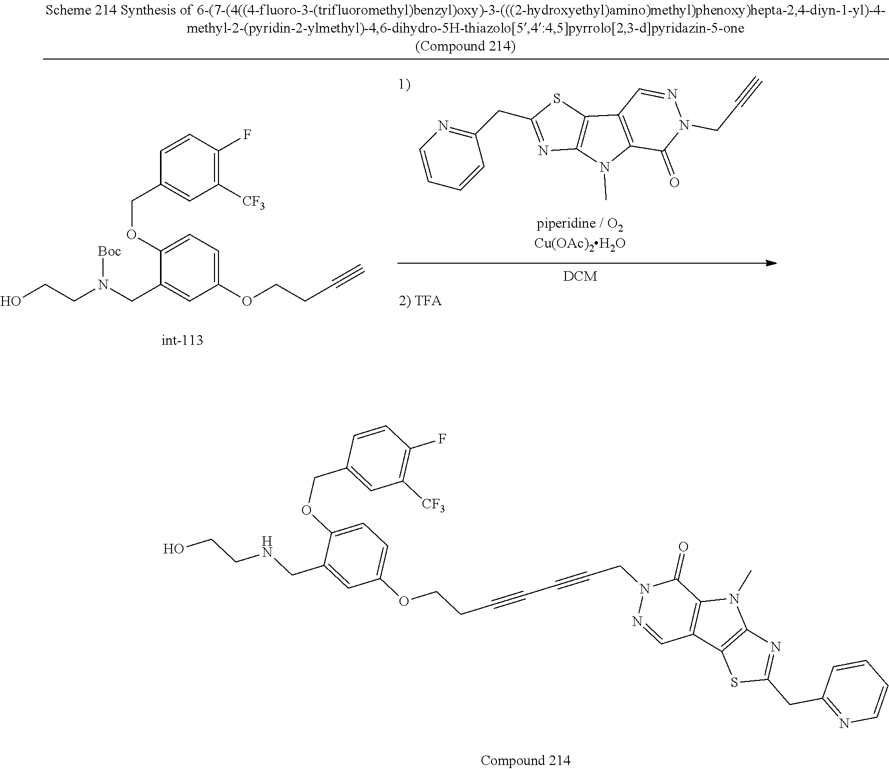 [SMILES: C#CCCOc1ccc(OCc2ccc(F)c(C(F)(F)F)c2)c(CN(CCO)C(=O)OC(C)(C)C)c1 ; C#CCn1ncc2c3sc(Cc4ccccn4)nc3n(C)c2c1=O ; Cn1c2nc(Cc3ccccn3)sc2c2cnn(CC#CC#CCCOc3ccc(OCc4ccc(F)c(C(F)(F)F)c4)c(CNCCO)c3)c(=O)c21]

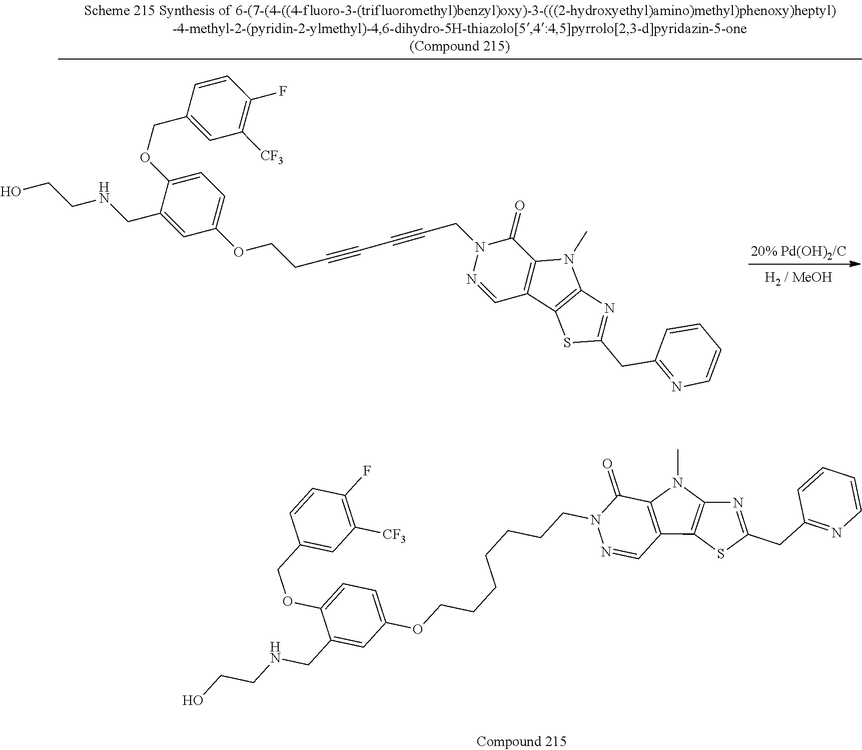 [SMILES: Cn1c2nc(Cc3ccccn3)sc2c2cnn(CC#CC#CCCOc3ccc(OCc4ccc(F)c(C(F)(F)F)c4)c(CNCCO)c3)c(=O)c21 ; "H2 / MeOH" ; Cn1c2nc(Cc3ccccn3)sc2c2cnn(CCCCCCCOc3ccc(OCc4ccc(F)c(C(F)(F)F)c4)c(CNCCO)c3)c(=O)c21]

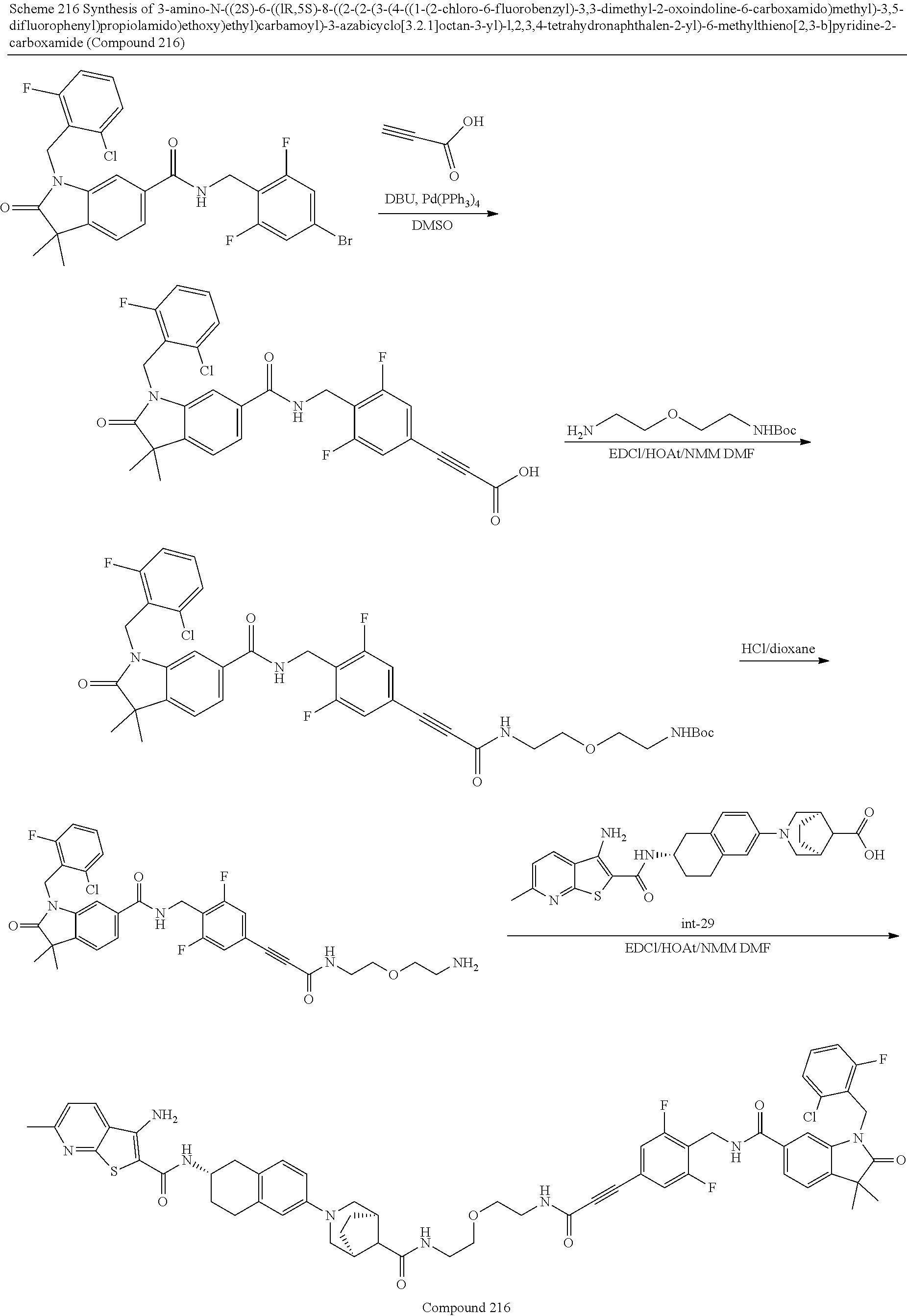

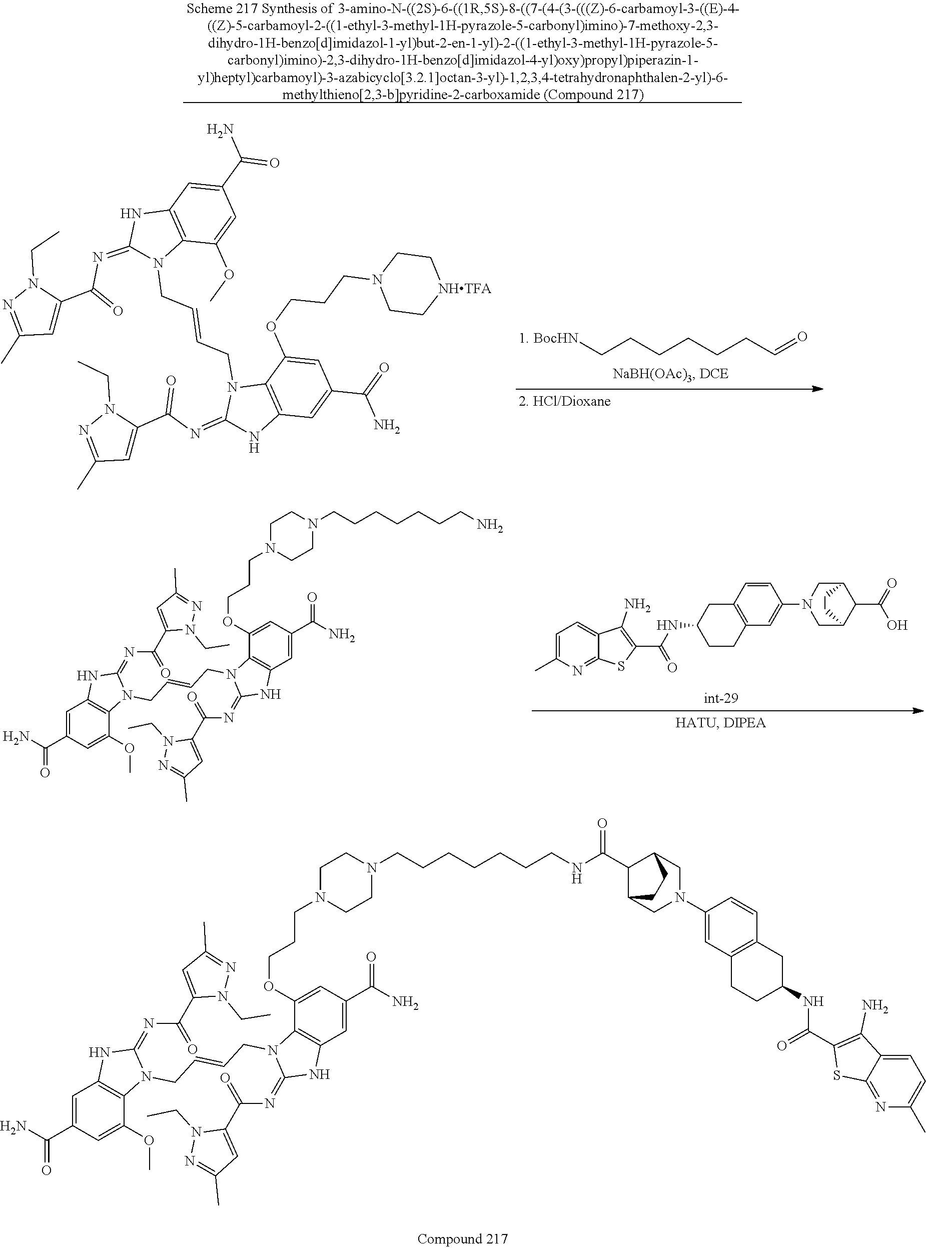

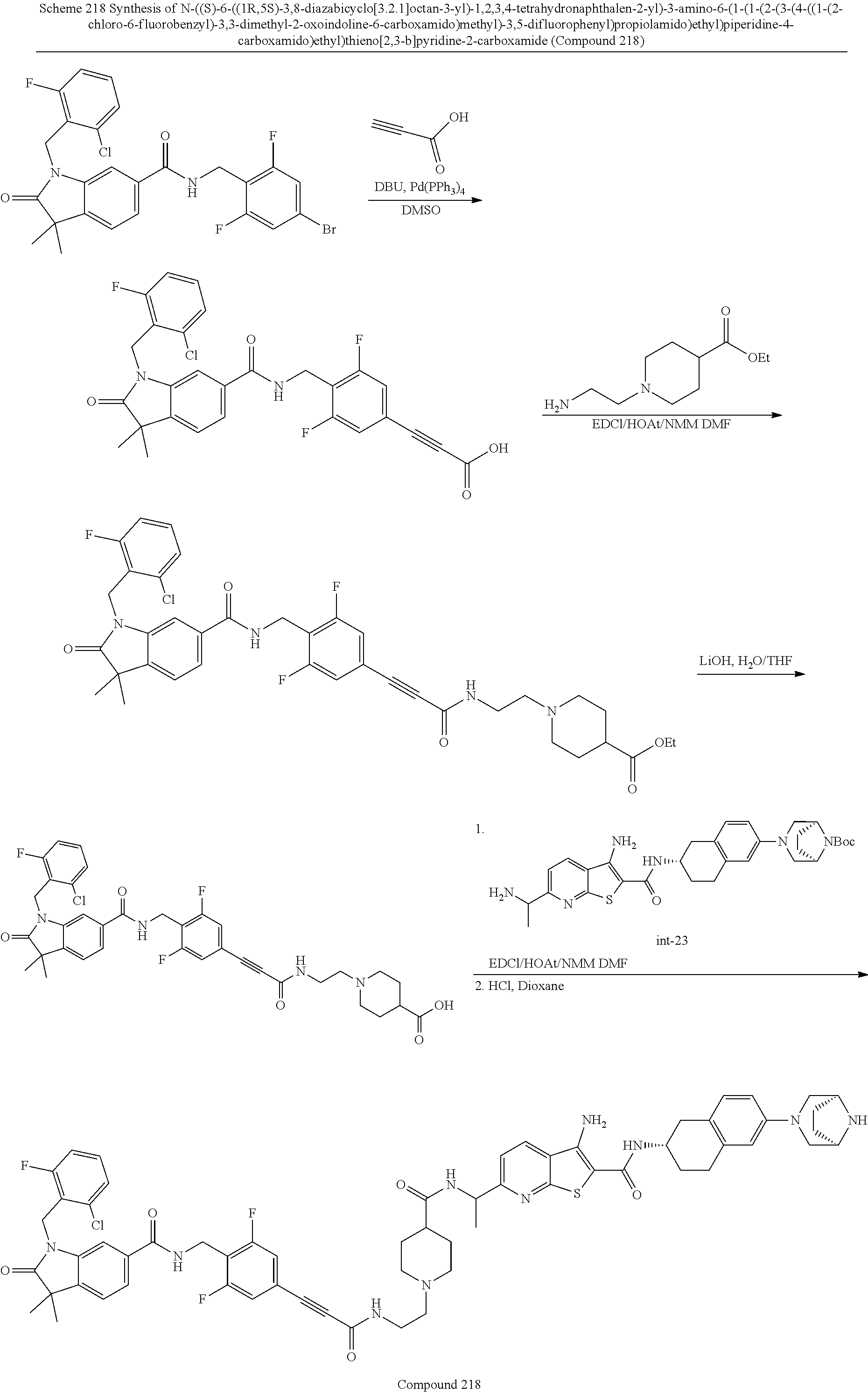

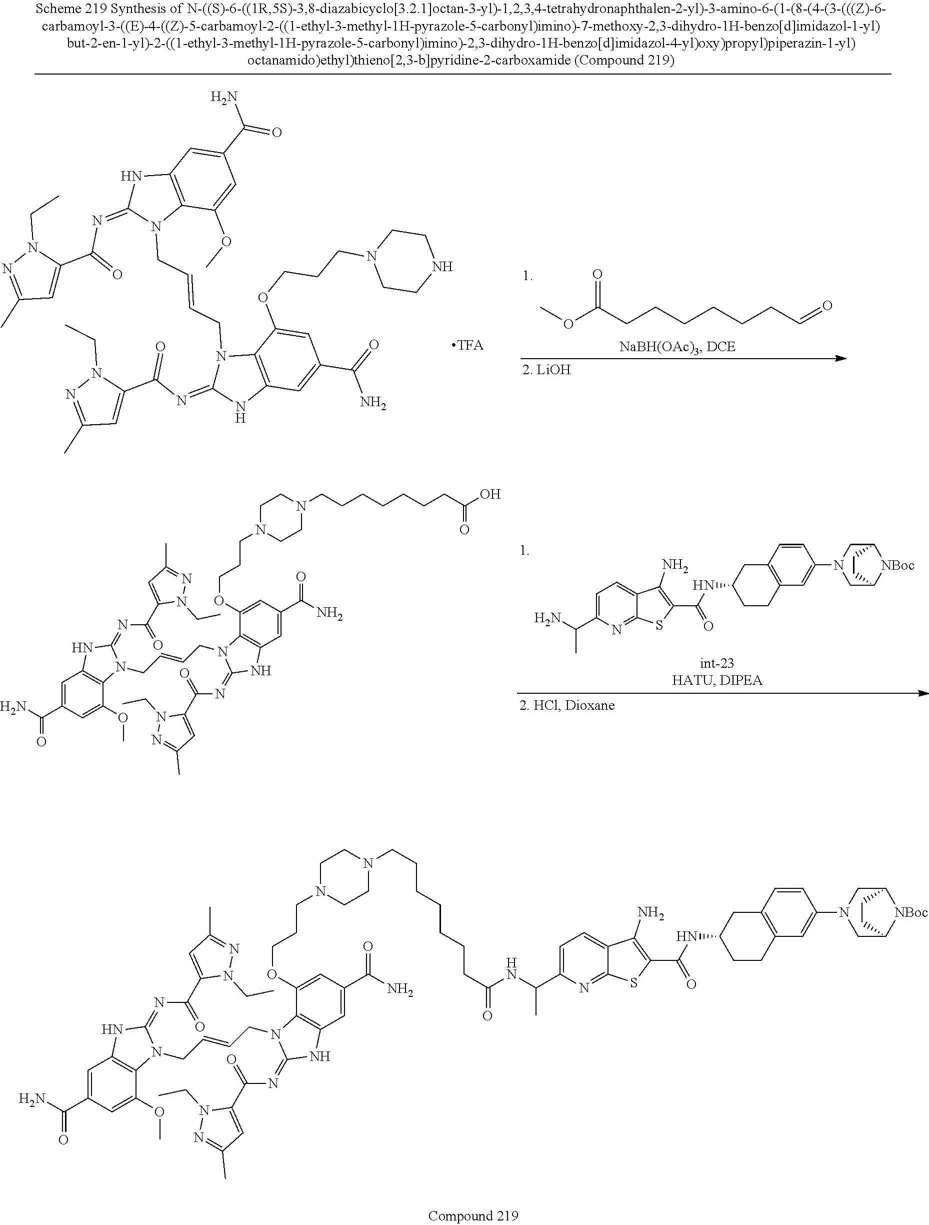

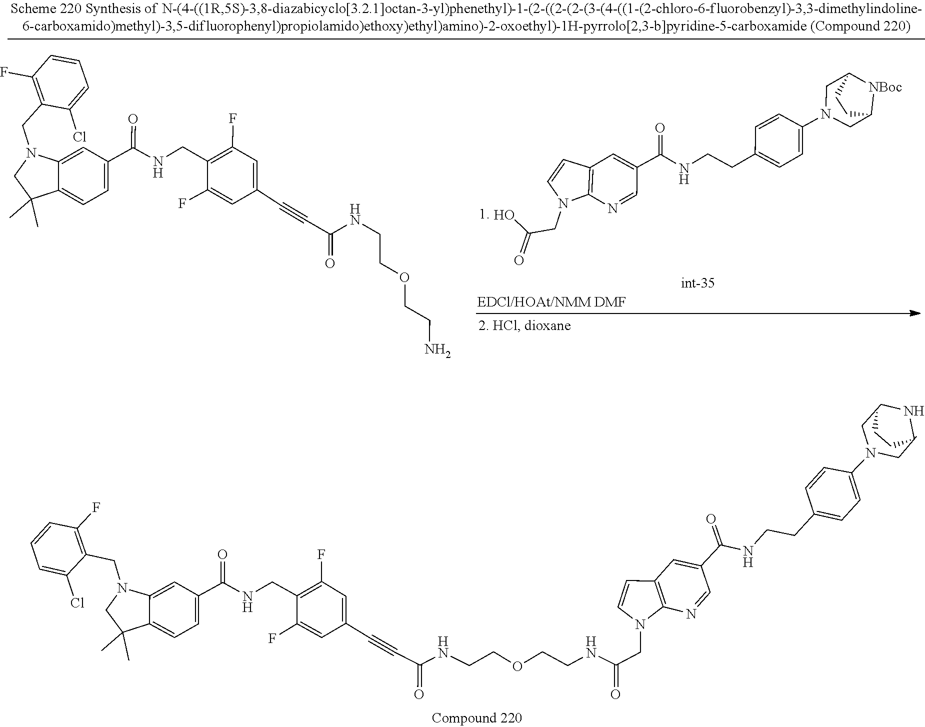 [SMILES: CC1(C)CN(Cc2c(F)cccc2Cl)c2cc(C(=O)NCc3c(F)cc(C#CC(=O)NCCOCCN)cc3F)ccc21 ; CC(C)(C)OC(=O)N1[C@@H]2CC[C@H]1CN(c1ccc(CCNC(=O)c3cnc4c(ccn4CC(=O)O)c3)cc1)C2 ; CC1(C)CN(Cc2c(F)cccc2Cl)c2cc(C(=O)NCc3c(F)cc(C#CC(=O)NCCOCCNC(=O)Cn4ccc5cc(C(=O)NCCc6ccc(N7C[C@@H]8CC[C@](C)(C7)NC8)cc6)cnc54)cc3F)ccc21]

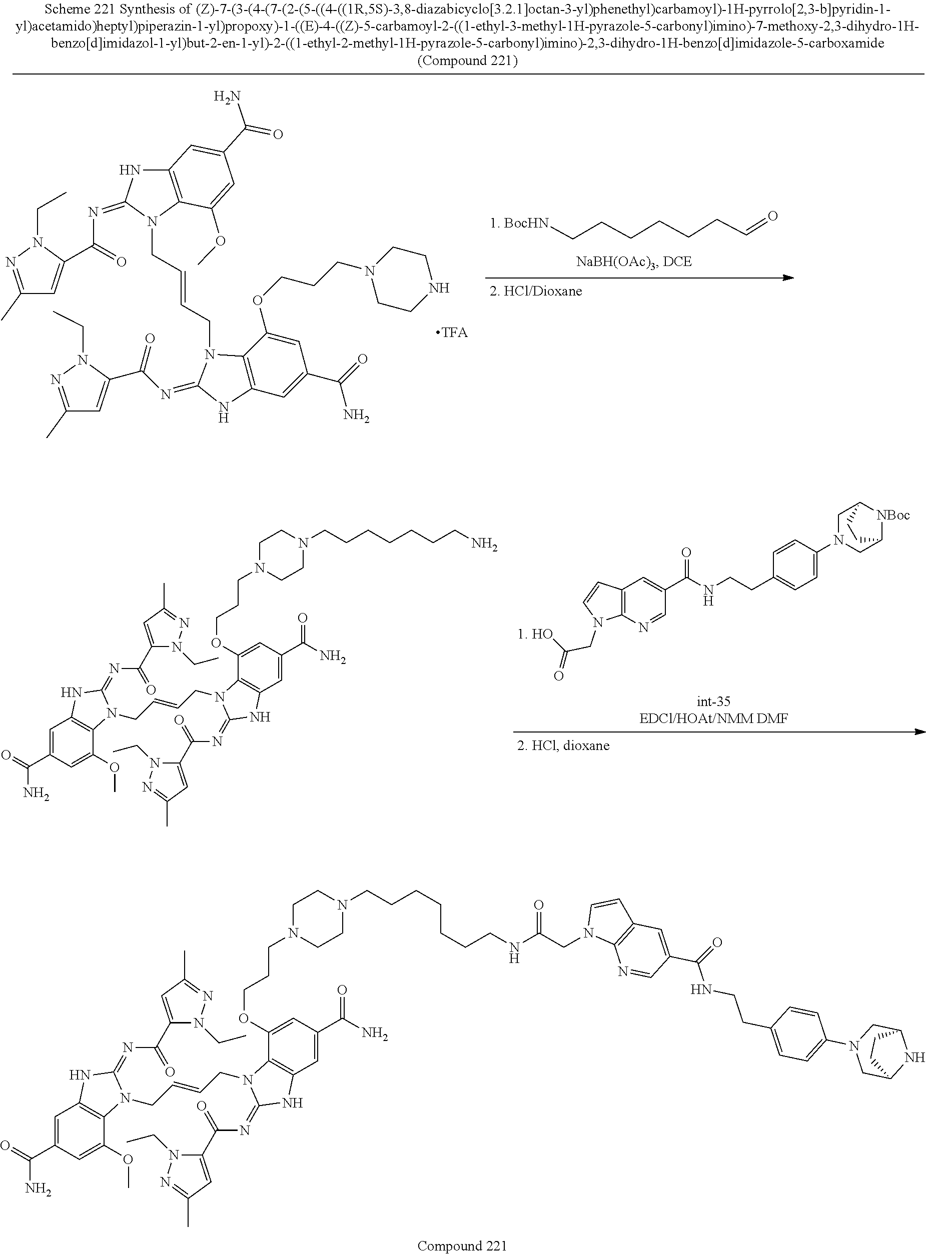

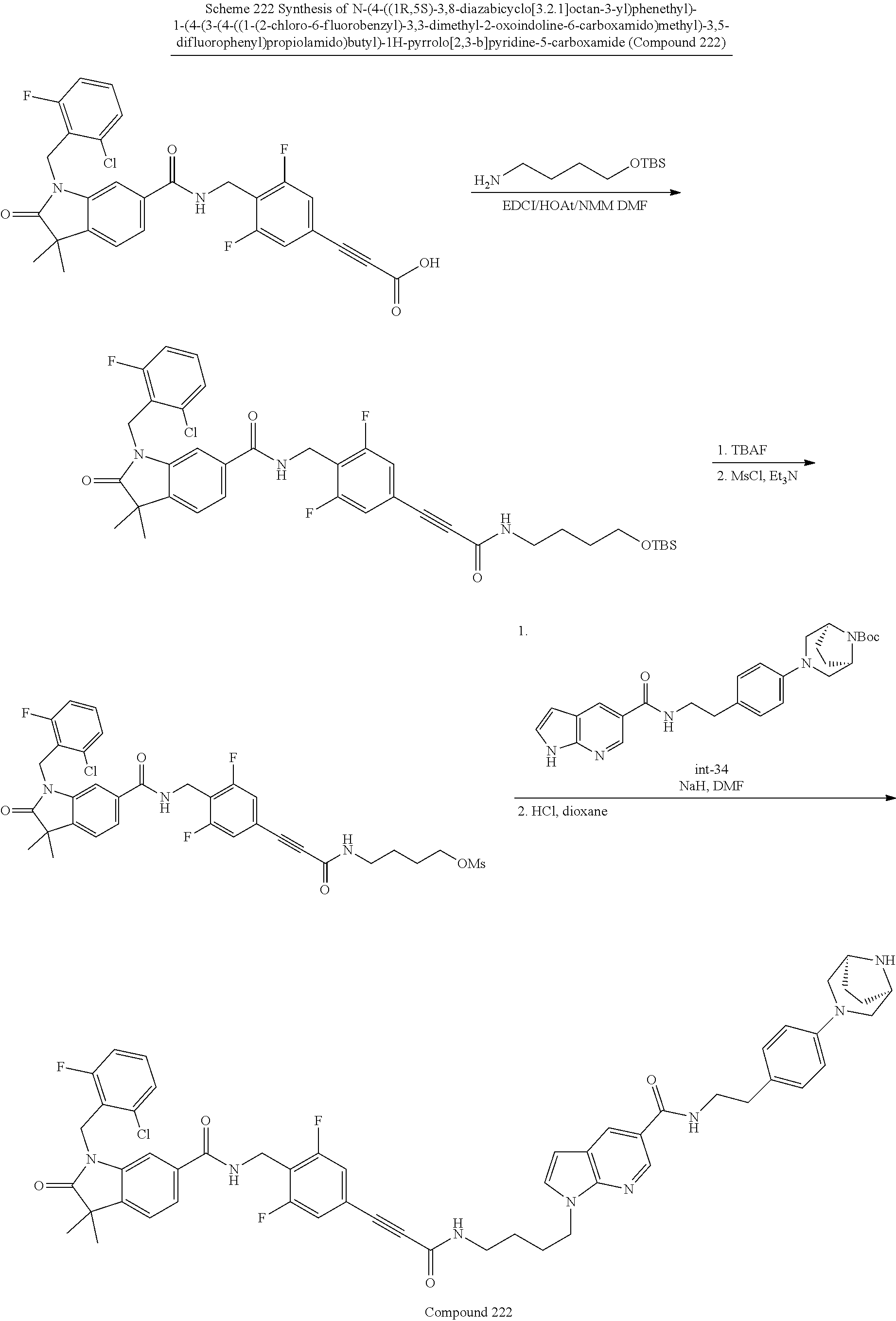 [SMILES: CC1(C)C(=O)N(Cc2c(F)cccc2Cl)c2cc(C(=O)NCc3c(F)cc(C#CC(=O)O)cc3F)ccc21 ; CC(C)(C)[Si](C)(C)OCCCCN ; CC1(C)C(=O)N(Cc2c(F)cccc2Cl)c2cc(C(=O)NCc3c(F)cc(C#CC(=O)NCCCCO[Si](C)(C)C(C)(C)C)cc3F)ccc21 ; CC1(C)C(=O)N(Cc2c(F)cccc2Cl)c2cc(C(=O)NCc3c(F)cc(C#CC(=O)NCCCCOS(C)(=O)=O)cc3F)ccc21 ; CC(C)(C)OC(=O)N1[C@@H]2CC[C@H]1CN(c1ccc(CCNC(=O)c3cnc4[nH]ccc4c3)cc1)C2 ; CC1(C)C(=O)N(Cc2c(F)cccc2Cl)c2cc(C(=O)NCc3c(F)cc(C#CC(=O)NCCCCn4ccc5cc(C(=O)NCCc6ccc(N7C[C@H]8CC[C@@H](C7)N8)cc6)cnc54)cc3F)ccc21]

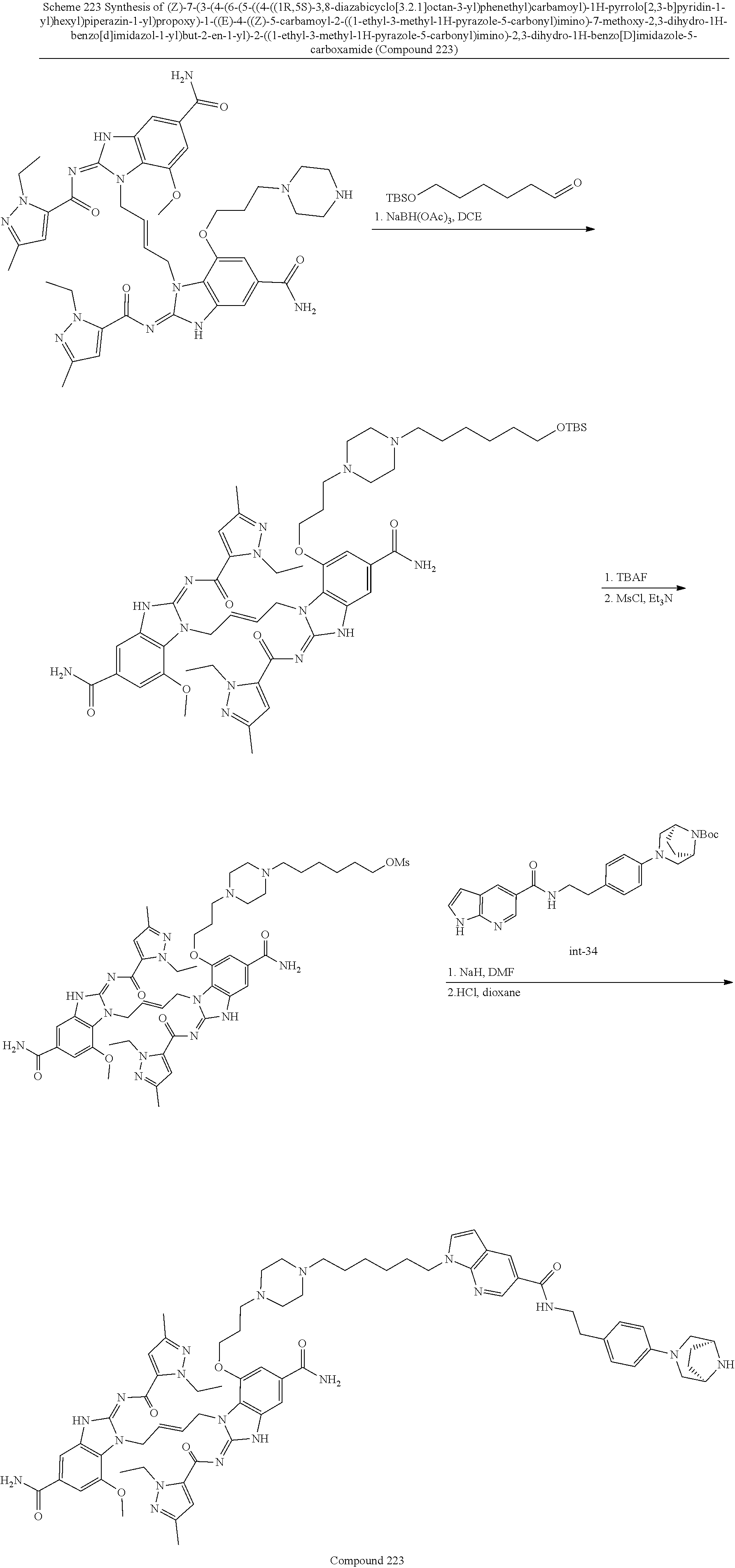

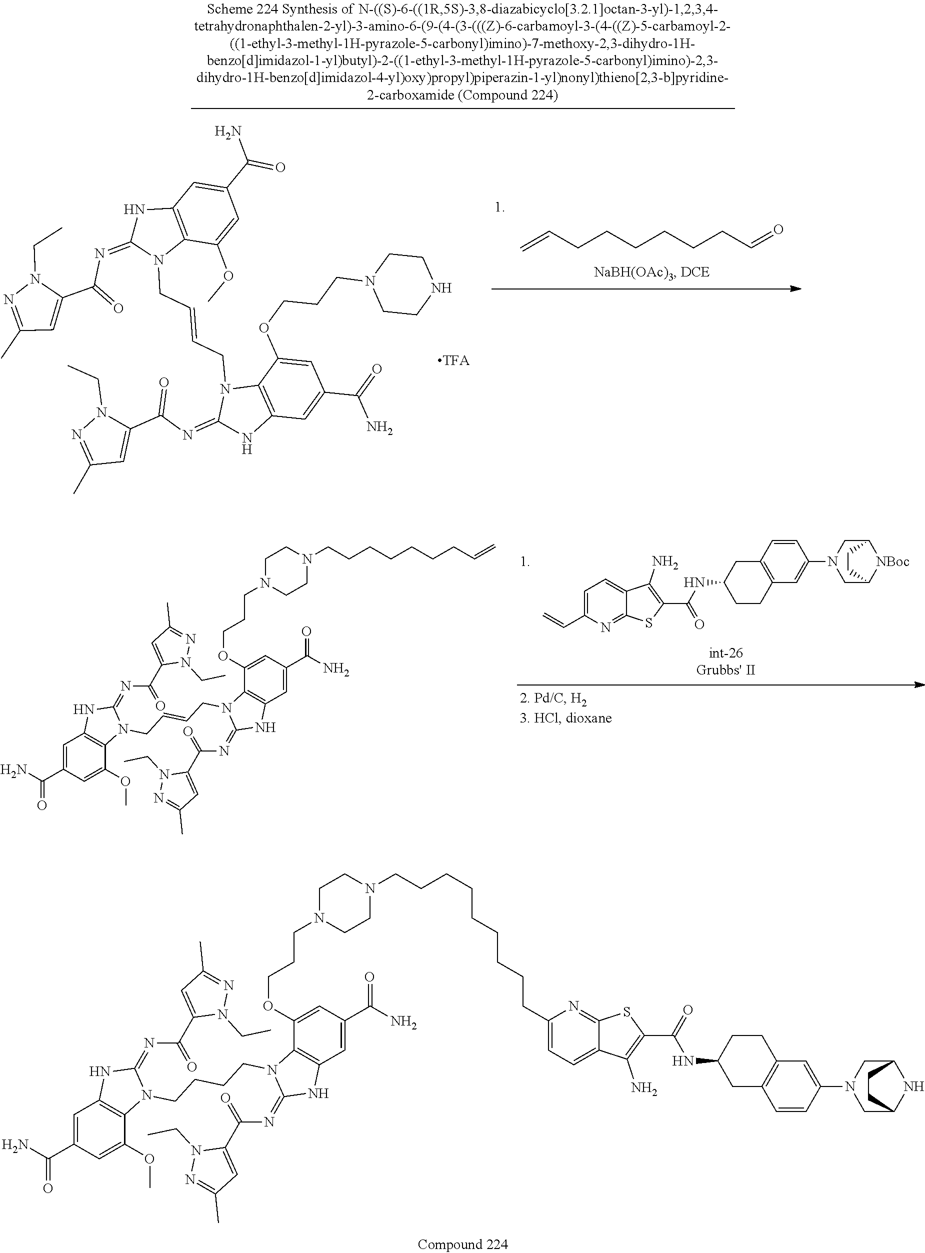

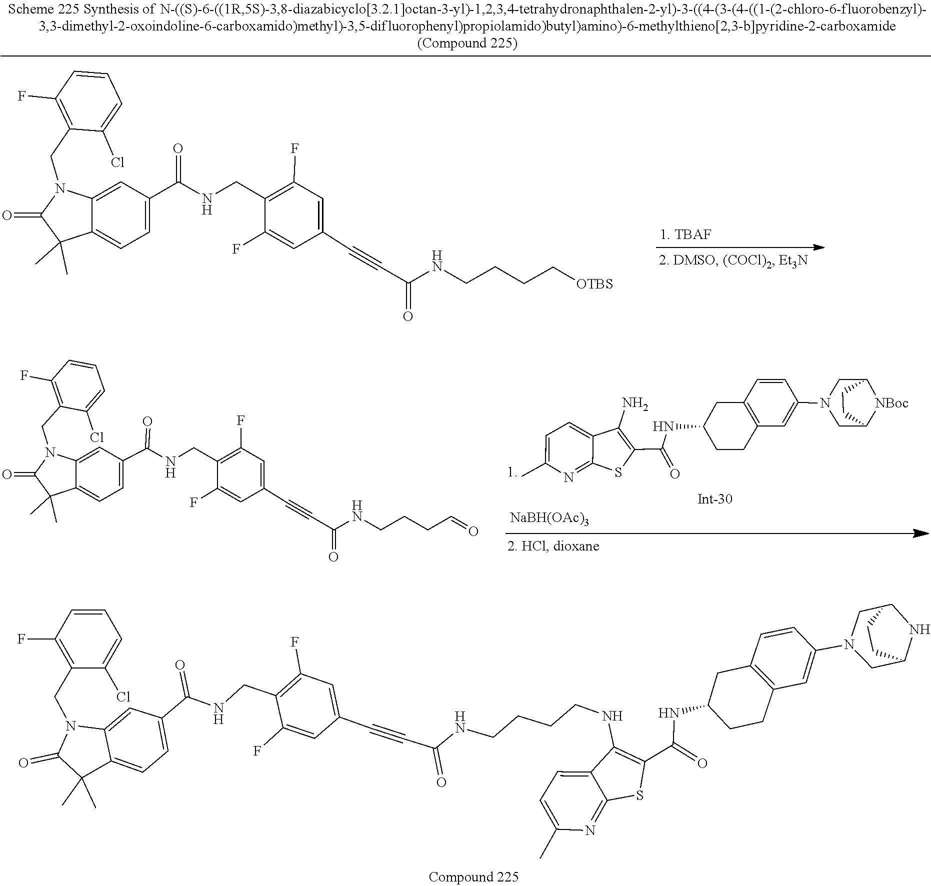 [SMILES: CC1(C)C(=O)N(Cc2c(F)cccc2Cl)c2cc(C(=O)NCc3c(F)cc(C#CC(=O)NCCCCO[Si](C)(C)C(C)(C)C)cc3F)ccc21 ; CC1(C)C(=O)N(Cc2c(F)cccc2Cl)c2cc(C(=O)NCc3c(F)cc(C#CC(=O)NCCCC=O)cc3F)ccc21 ; Cc1ccc2c(N)c(C(=O)N[C@H]3CCc4cc(N5C[C@H]6CC[C@@H](C5)N6C(=O)OC(C)(C)C)ccc4C3)sc2n1 ; Cc1ccc2c(NCCCCNC(=O)C#Cc3cc(F)c(CNC(=O)c4ccc5c(c4)N(Cc4c(F)cccc4Cl)C(=O)C5(C)C)c(F)c3)c(C(=O)N[C@H]3CCc4cc(N5C[C@@H]6CC[C@H](C5)NC6)ccc4C3)sc2n1 ; "NaBH(OAc)3"]

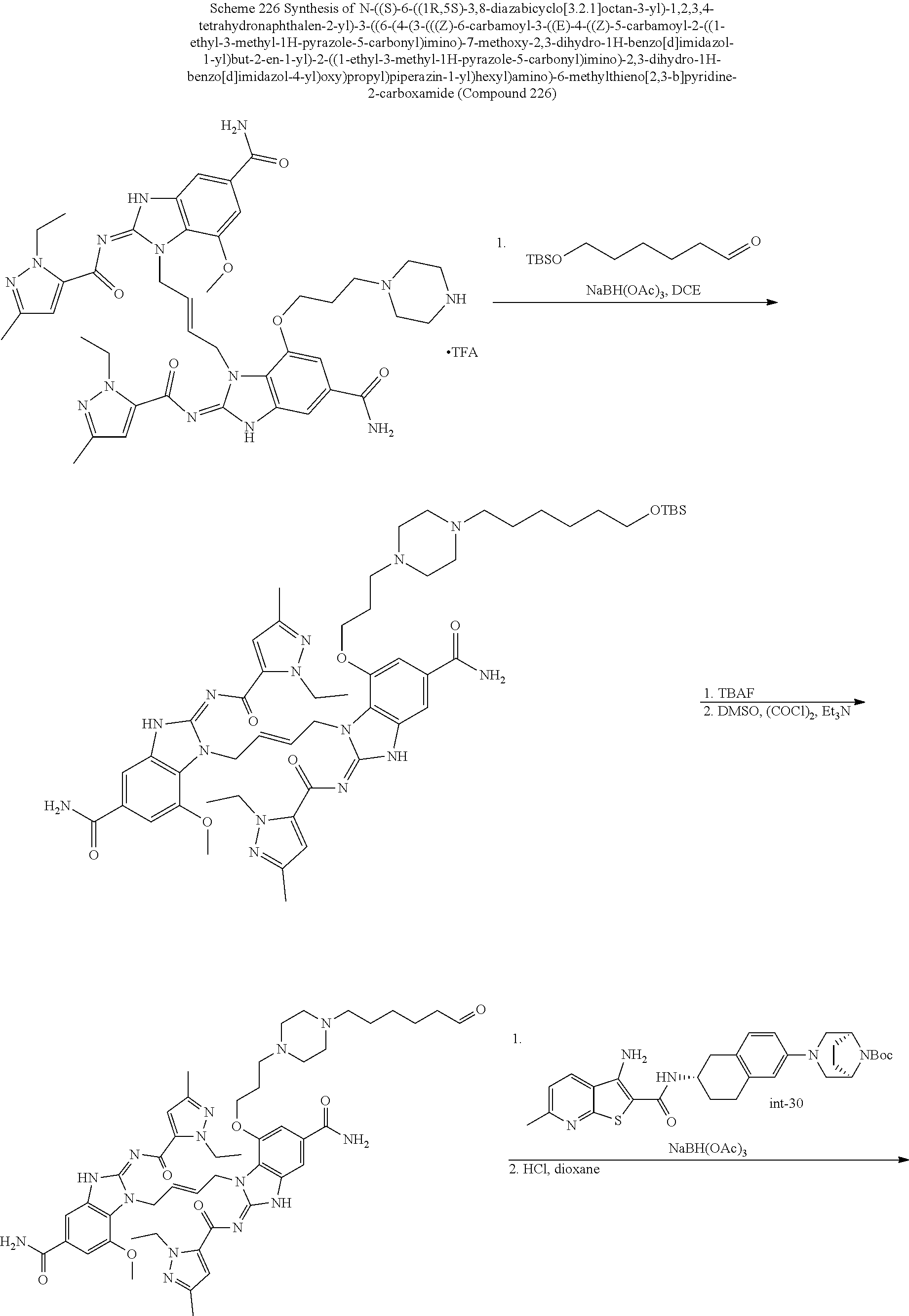

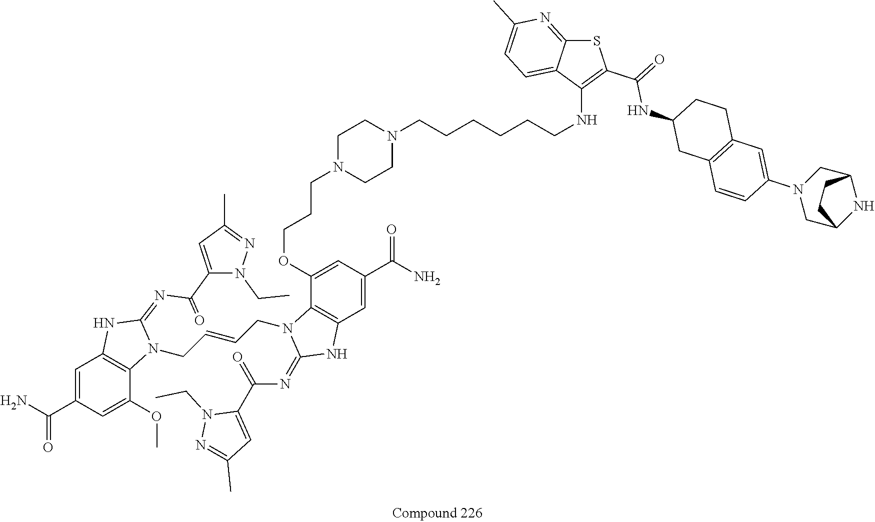 [SMILES: CCn1nc(C)cc1C(=O)/N=c1/[nH]c2cc(C(N)=O)cc(OC)c2n1C/C=C/Cn1/c(=N\C(=O)c2cc(C)nn2CC)[nH]c2cc(C(N)=O)cc(OCCCN3CCN(CCCCCCNc4c(C(=O)N[C@H]5CCc6cc(N7C[C@H]8CC[C@@H](C7)N8)ccc6C5)sc5nc(C)ccc45)CC3)c21]

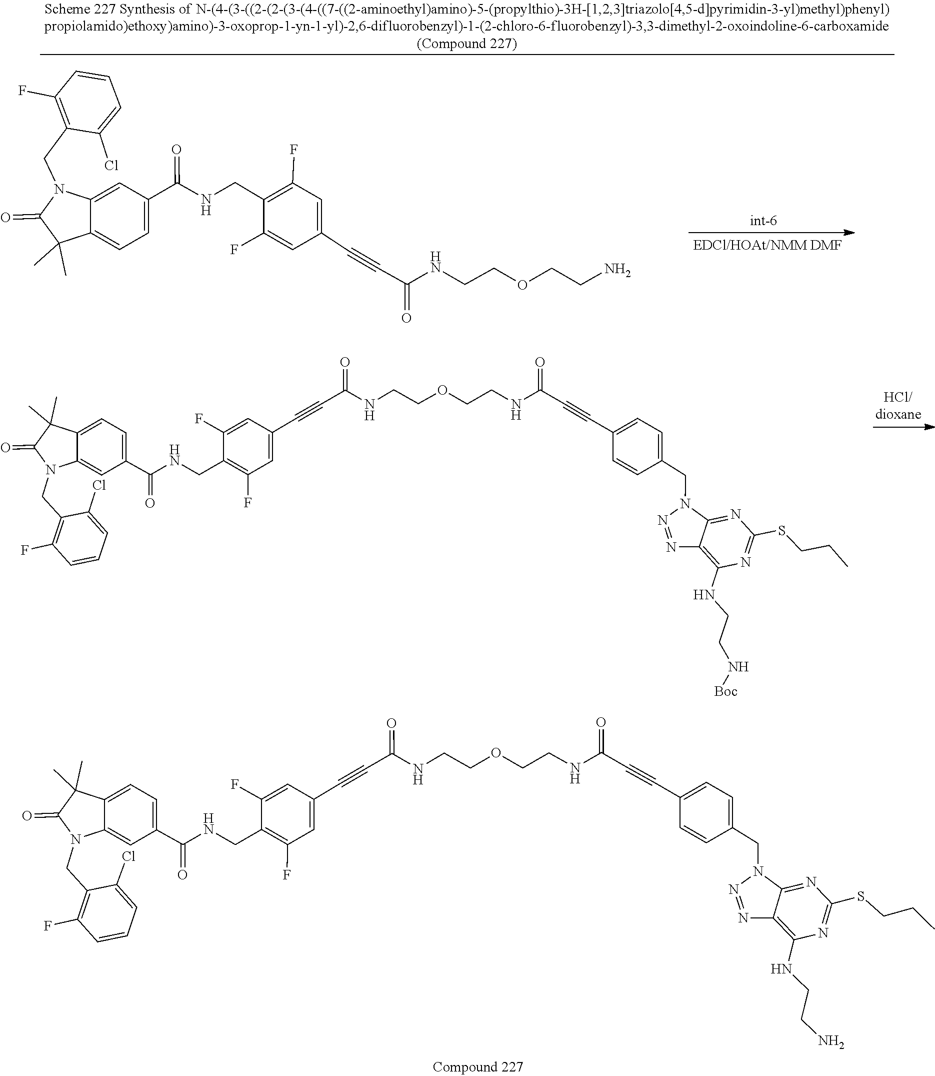 [SMILES: CC1(C)C(=O)N(Cc2c(F)cccc2Cl)c2cc(C(=O)NCc3c(F)cc(C#CC(=O)NCCOCCN)cc3F)ccc21 ; CCCSc1nc(NCCNC(=O)OC(C)(C)C)c2nnn(Cc3ccc(C#CC(=O)NCCOCCNC(=O)C#Cc4cc(F)c(CNC(=O)c5ccc6c(c5)N(Cc5c(F)cccc5Cl)C(=O)C6(C)C)c(F)c4)cc3)c2n1 ; CCCSc1nc(NCCN)c2nnn(Cc3ccc(C#CC(=O)NCCOCCNC(=O)C#Cc4cc(F)c(CNC(=O)c5ccc6c(c5)N(Cc5c(F)cccc5Cl)C(=O)C6(C)C)c(F)c4)cc3)c2n1]

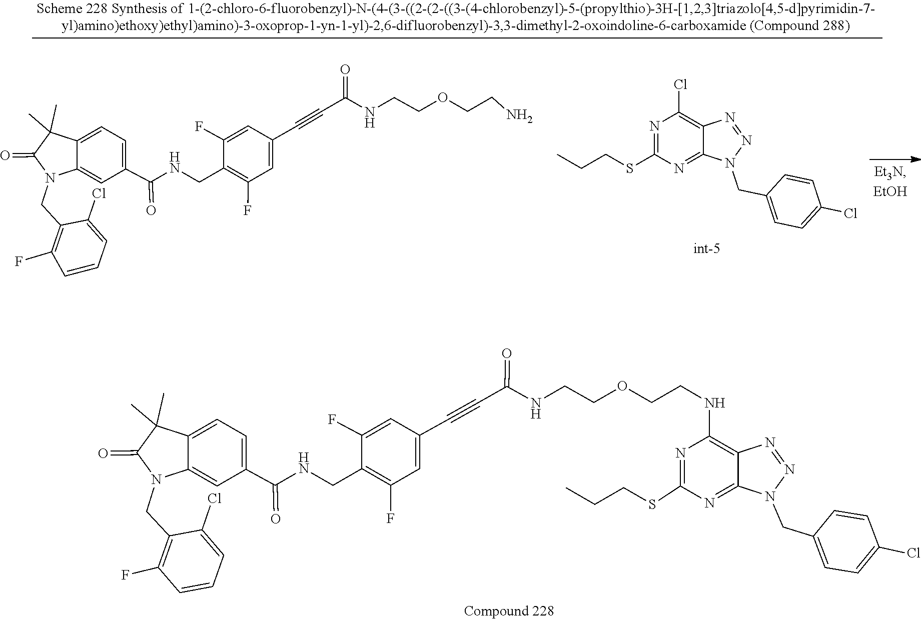 [SMILES: CC1(C)C(=O)N(Cc2c(F)cccc2Cl)c2cc(C(=O)NCc3c(F)cc(C#CC(=O)NCCOCCN)cc3F)ccc21 ; CCCSc1nc(Cl)c2nnn(Cc3ccc(Cl)cc3)c2n1 ; CCCSc1nc(NCCOCCNC(=O)C#Cc2cc(F)c(CNC(=O)c3ccc4c(c3)N(Cc3c(F)cccc3Cl)C(=O)C4(C)C)c(F)c2)c2nnn(Cc3ccc(Cl)cc3)c2n1]

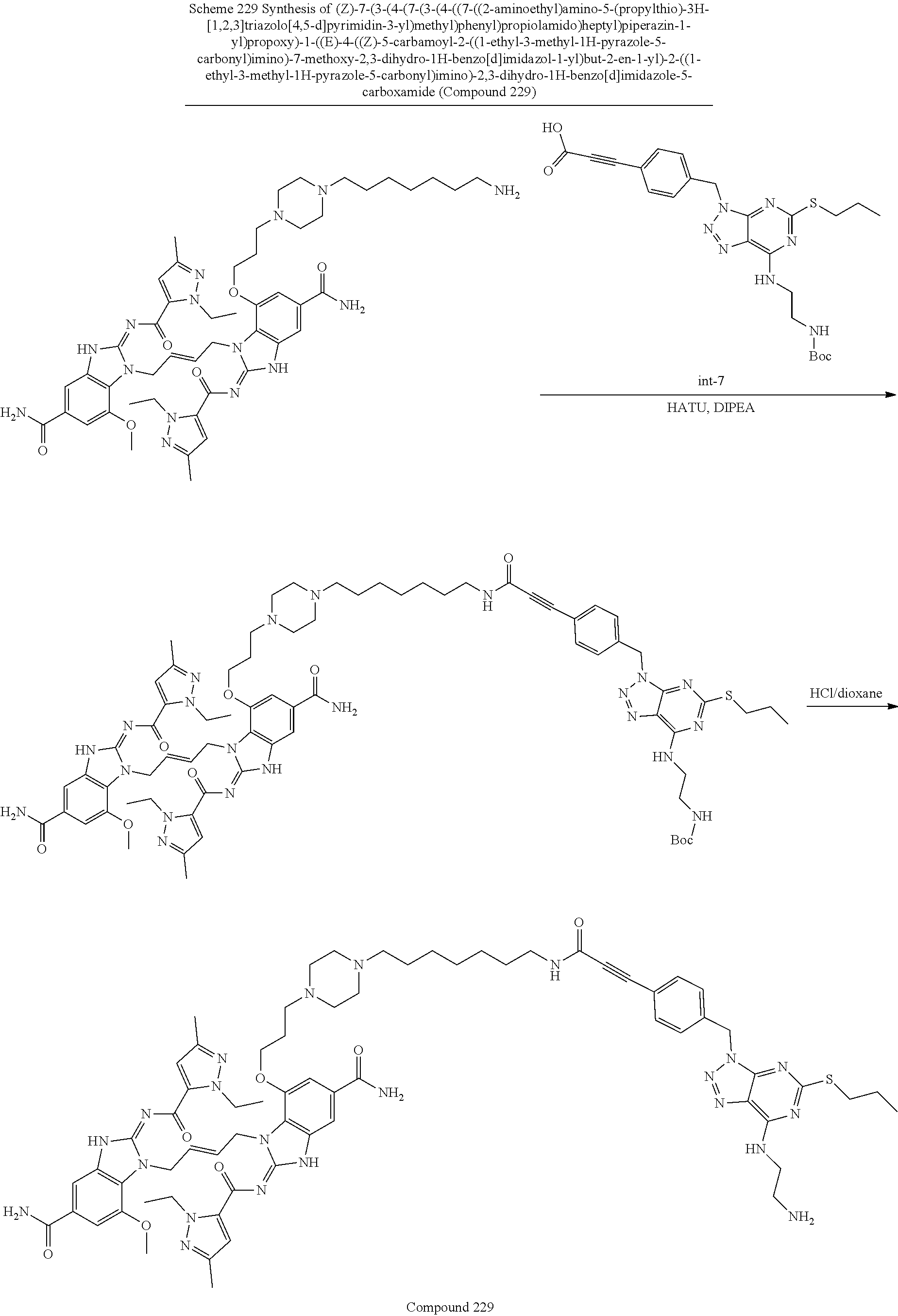

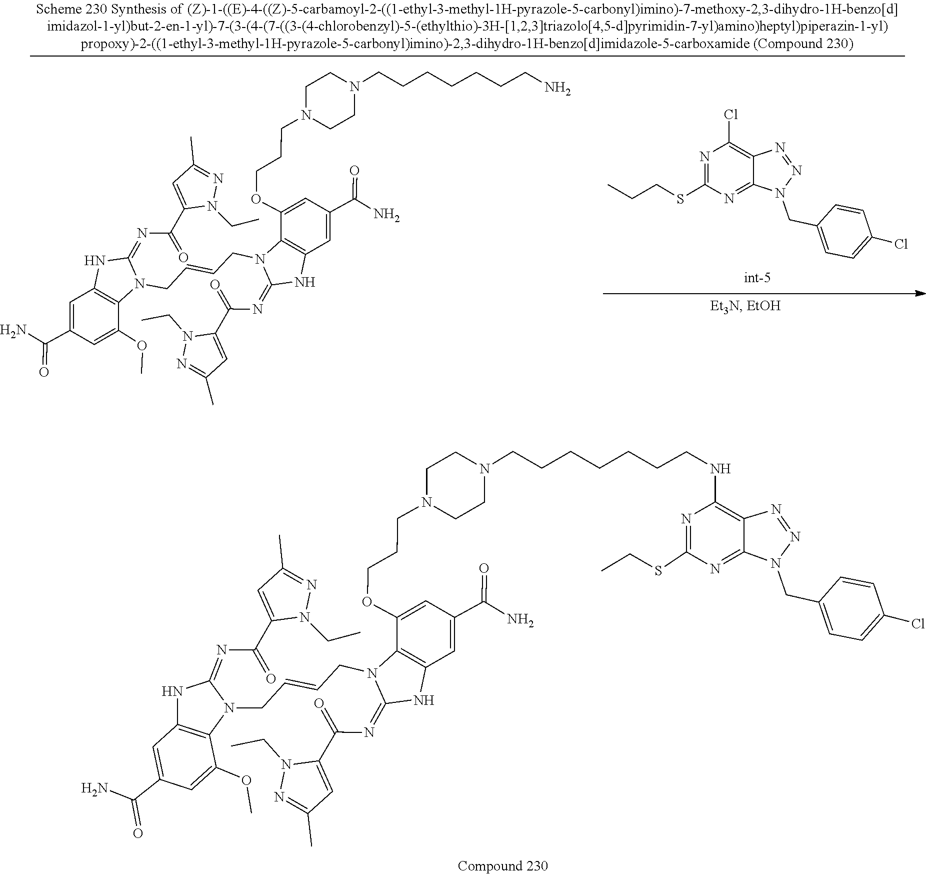

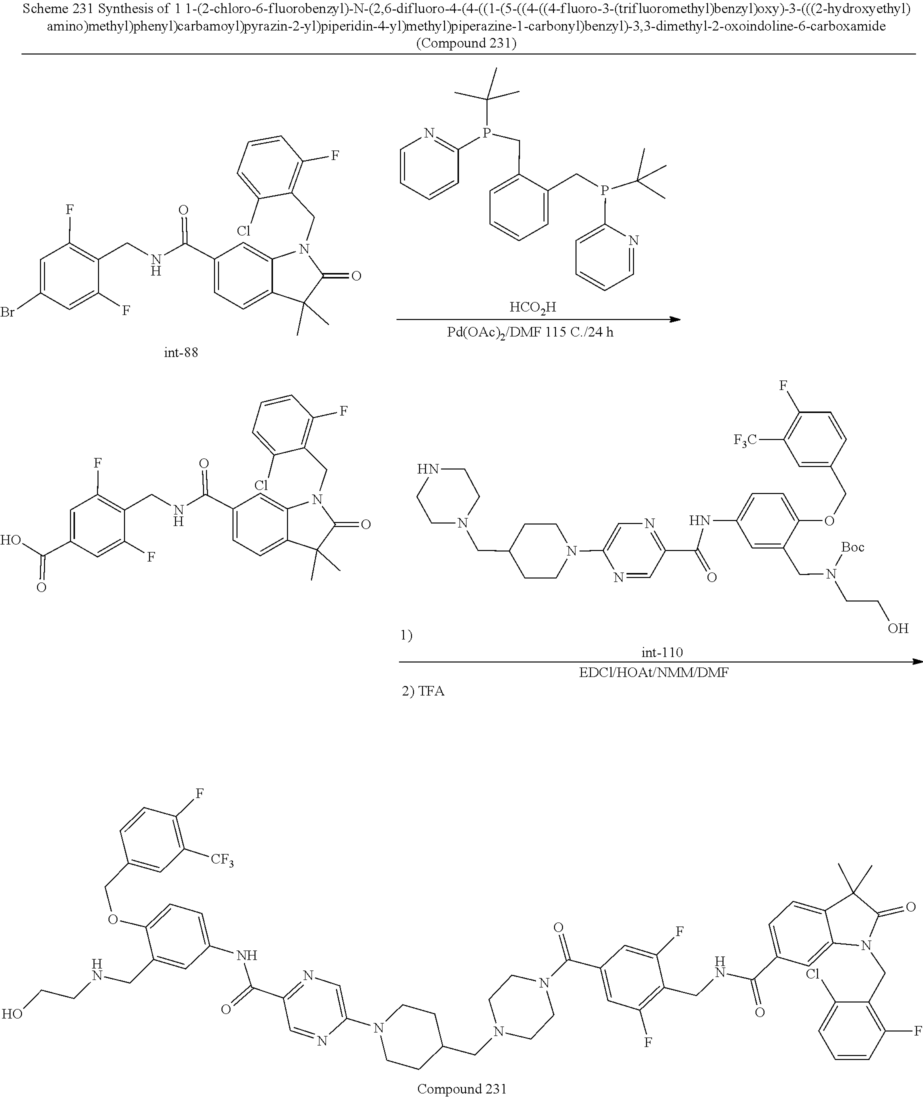

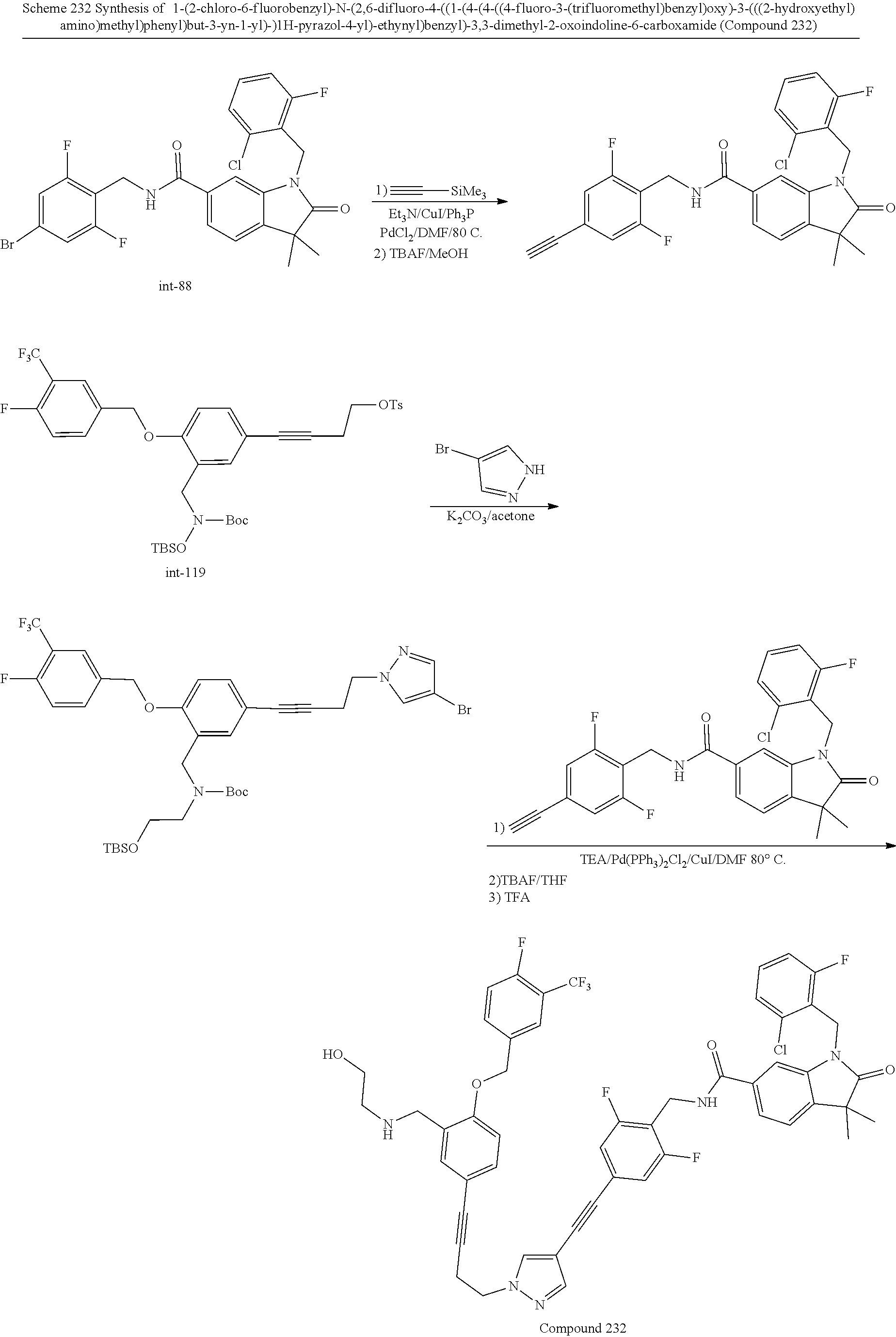

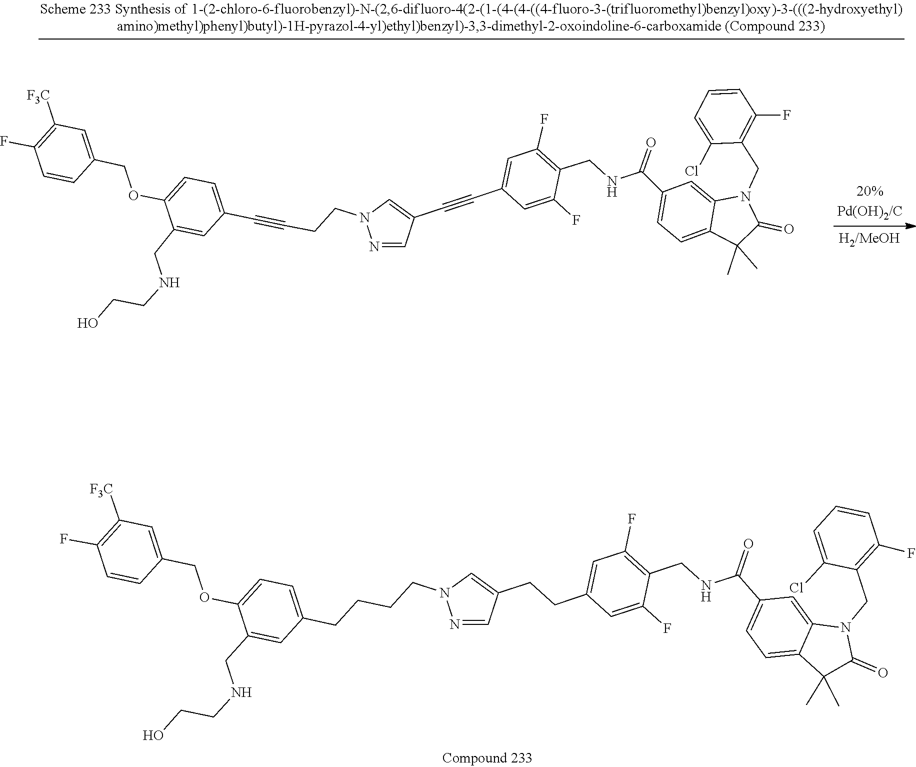 [SMILES: CC1(C)C(=O)N(Cc2c(F)cccc2Cl)c2cc(C(=O)NCc3c(F)cc(C#Cc4cnn(CCC#Cc5ccc(OCc6ccc(F)c(C(F)(F)F)c6)c(CNCCO)c5)c4)cc3F)ccc21 ; CC1(C)C(=O)N(Cc2c(F)cccc2Cl)c2cc(C(=O)NCc3c(F)cc(CCc4cnn(CCCCc5ccc(OCc6ccc(F)c(C(F)(F)F)c6)c(CNCCO)c5)c4)cc3F)ccc21]

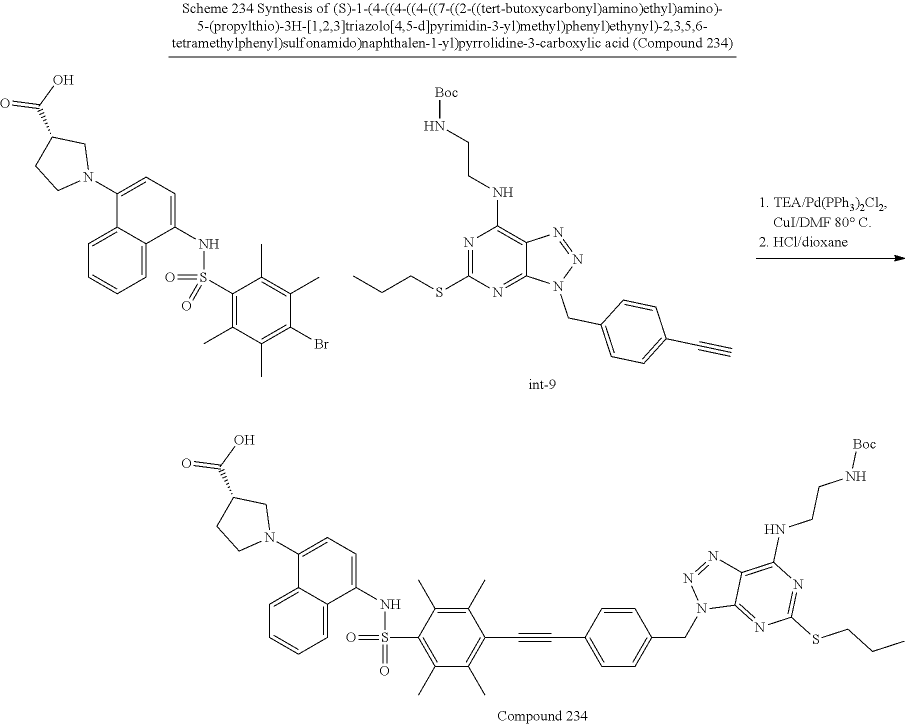

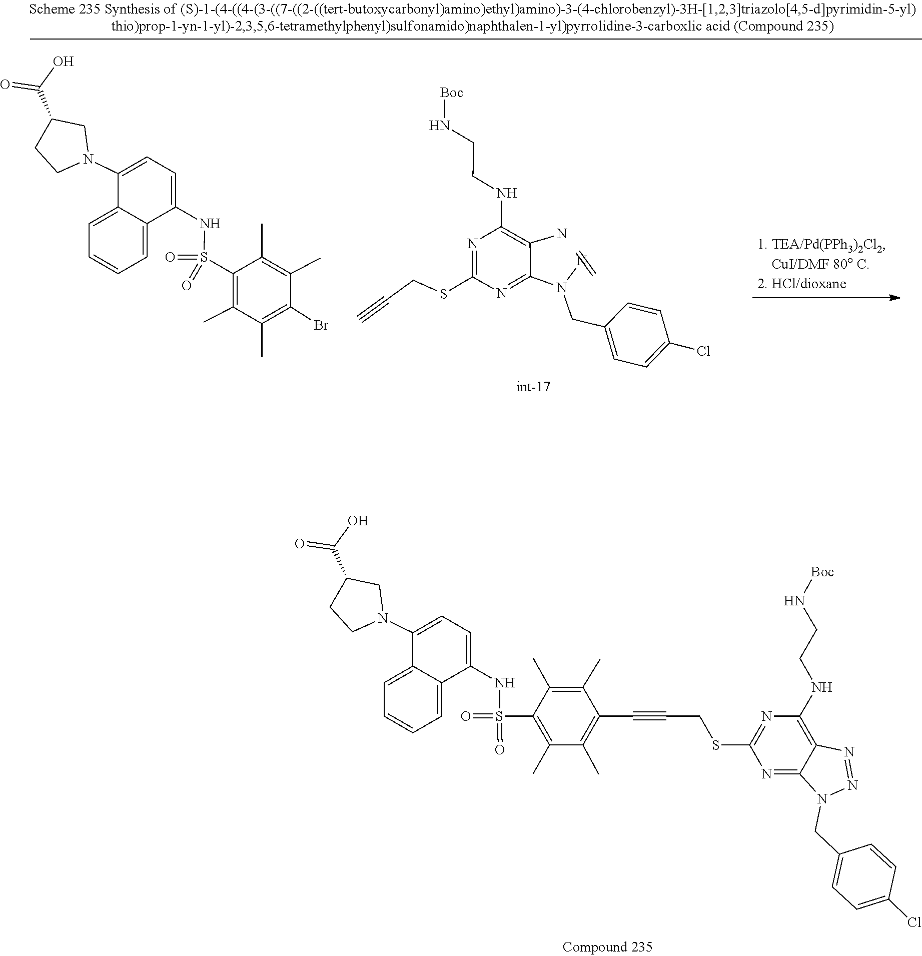

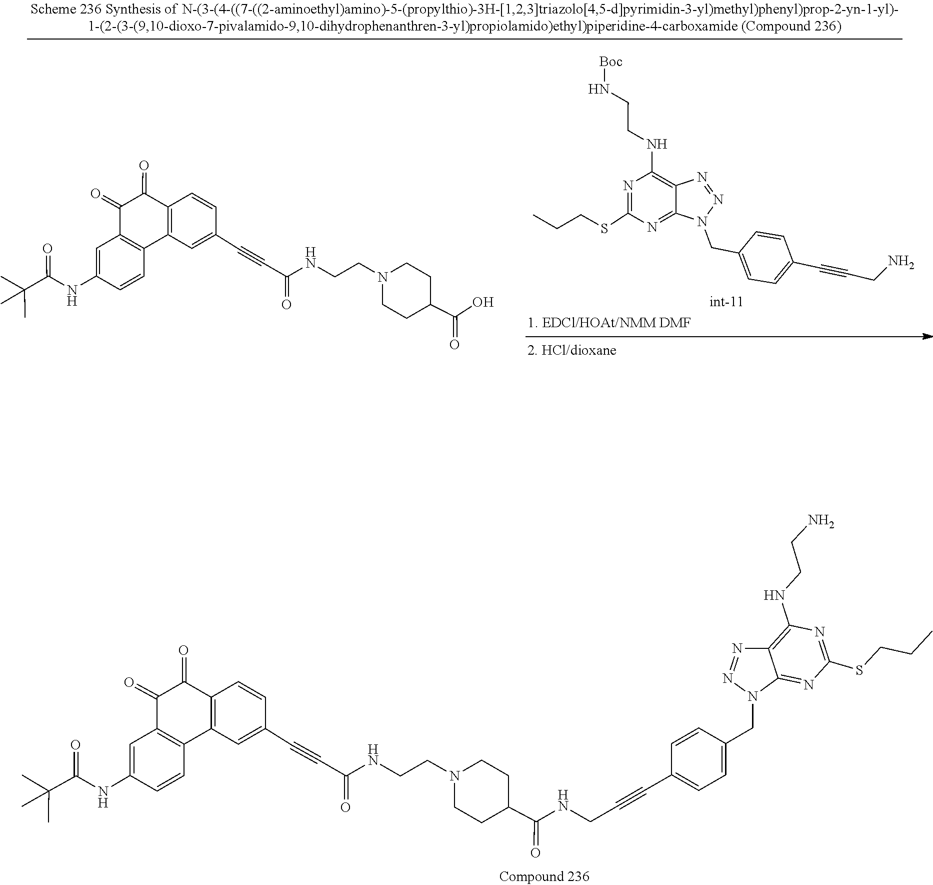 [SMILES: CC(C)(C)C(=O)Nc1ccc2c(c1)C(=O)C(=O)c1ccc(C#CC(=O)NCCN3CCC(C(=O)O)CC3)cc1-2 ; CCCSc1nc(NCCNC(=O)OC(C)(C)C)c2nnn(Cc3ccc(C#CCN)cc3)c2n1 ; CCCSc1nc(NCCN)c2nnn(Cc3ccc(C#CCNC(=O)C4CCN(CCNC(=O)C#Cc5ccc6c(c5)-c5ccc(NC(=O)C(C)(C)C)cc5C(=O)C6=O)CC4)cc3)c2n1]

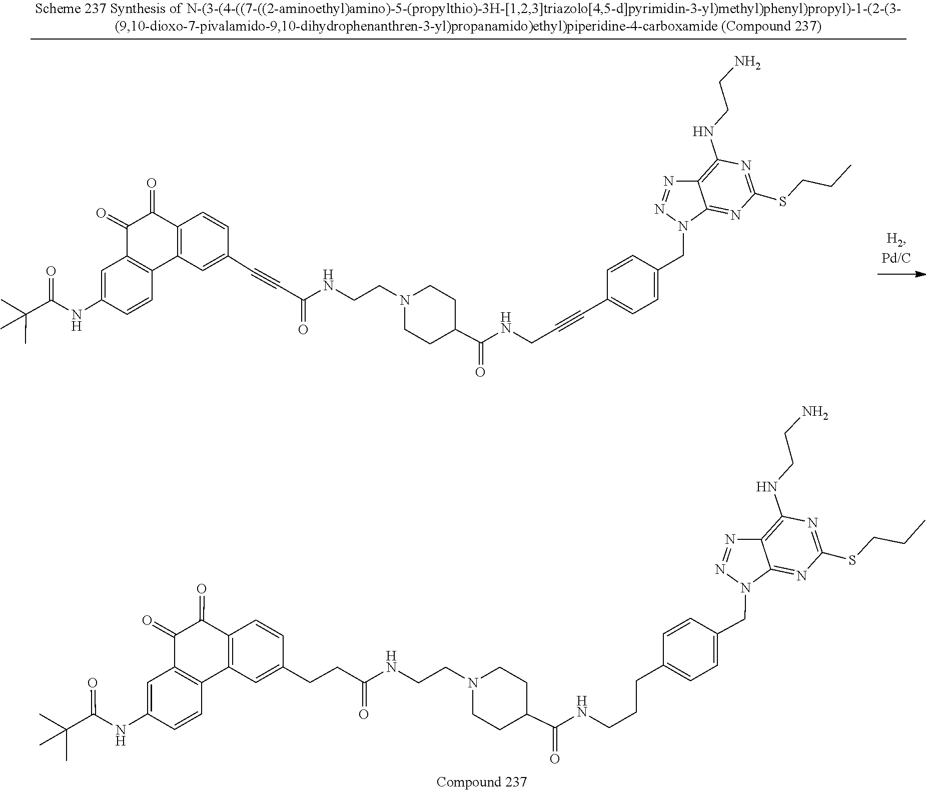

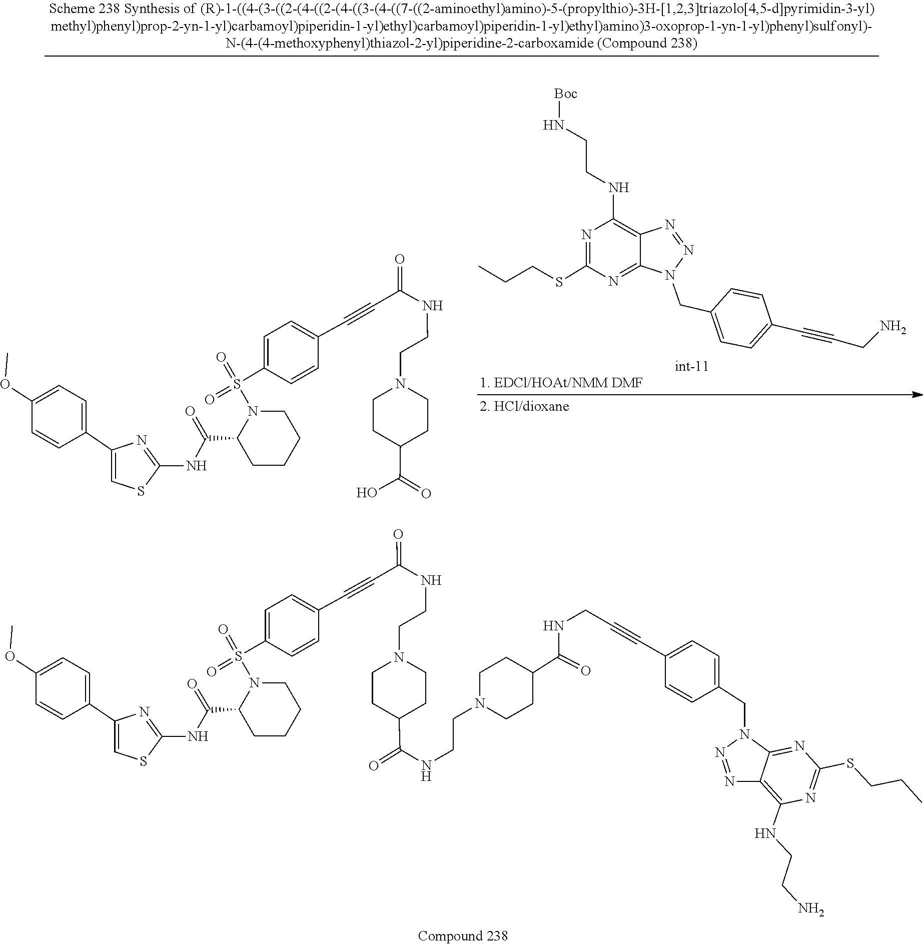 [SMILES: COc1ccc(-c2csc(NC(=O)[C@H]3CCCCN3S(=O)(=O)c3ccc(C#CC(=O)NCCN4CCC(C(=O)O)CC4)cc3)n2)cc1 ; CCCSc1nc(NCCNC(=O)OC(C)(C)C)c2nnn(Cc3ccc(C#CCN)cc3)c2n1 ; CCCSc1nc(NCCN)c2nnn(Cc3ccc(C#CCNC(=O)C4CCN(CCNC(=O)C5CCN(CCNC(=O)C#Cc6ccc(S(=O)(=O)N7CCCC[C@@H]7C(=O)Nc7nc(-c8ccc(OC)cc8)cs7)cc6)CC5)CC4)cc3)c2n1]

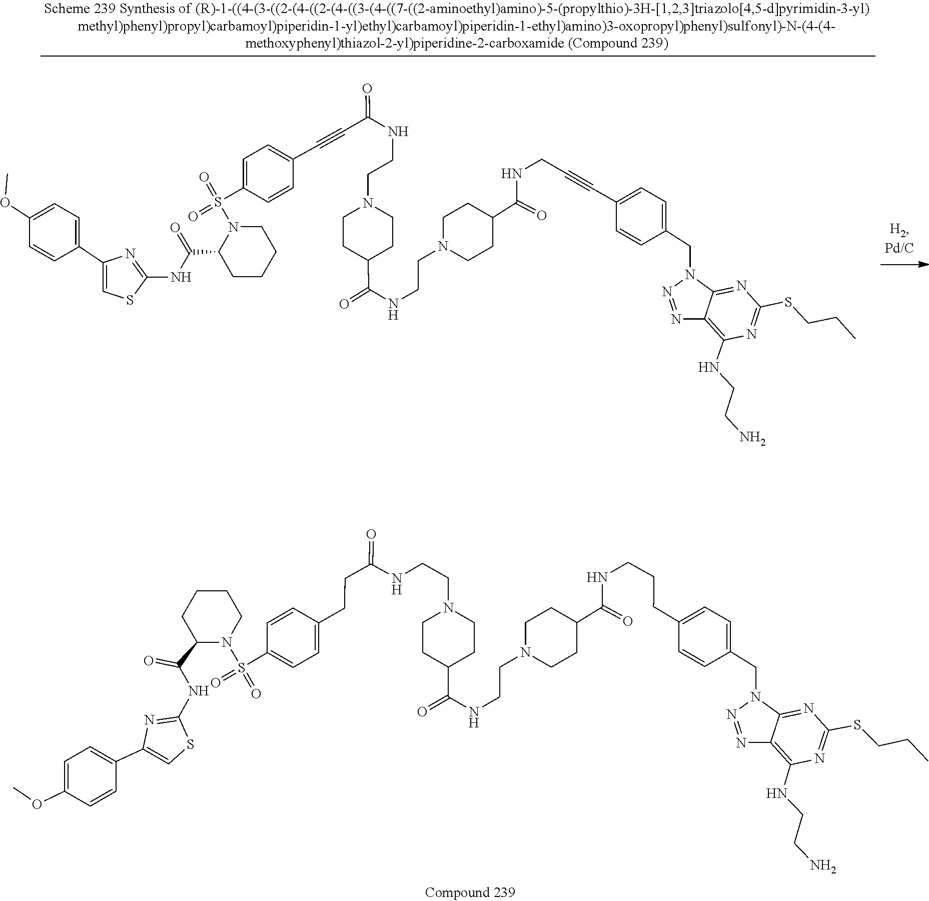

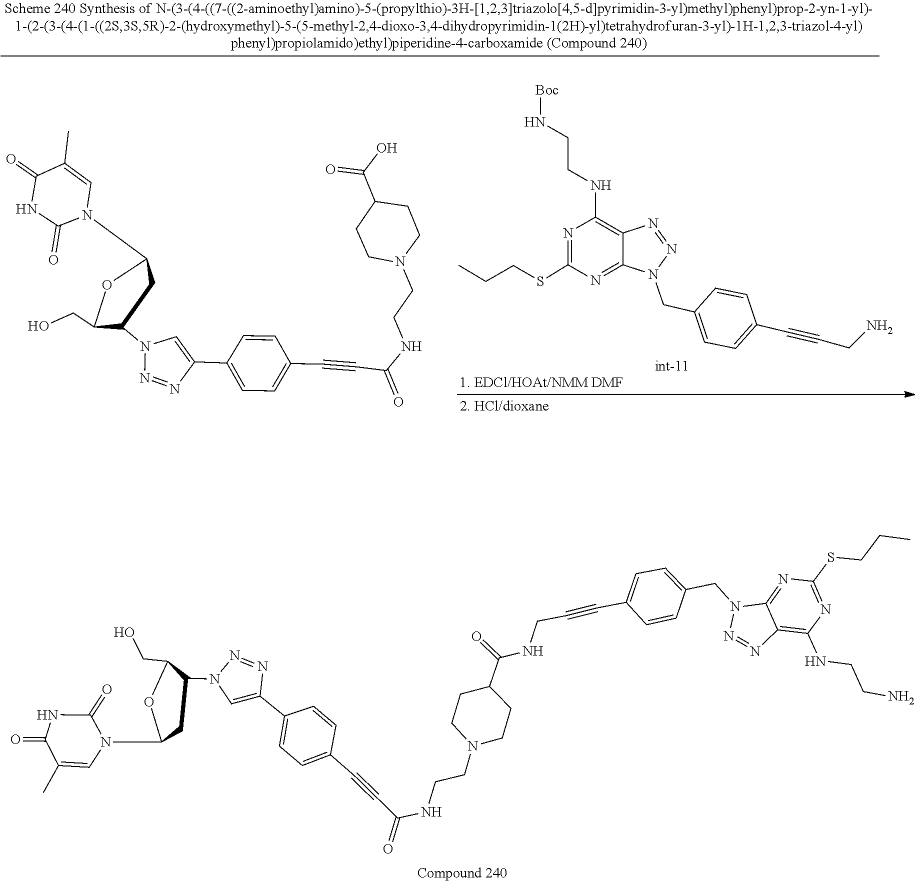

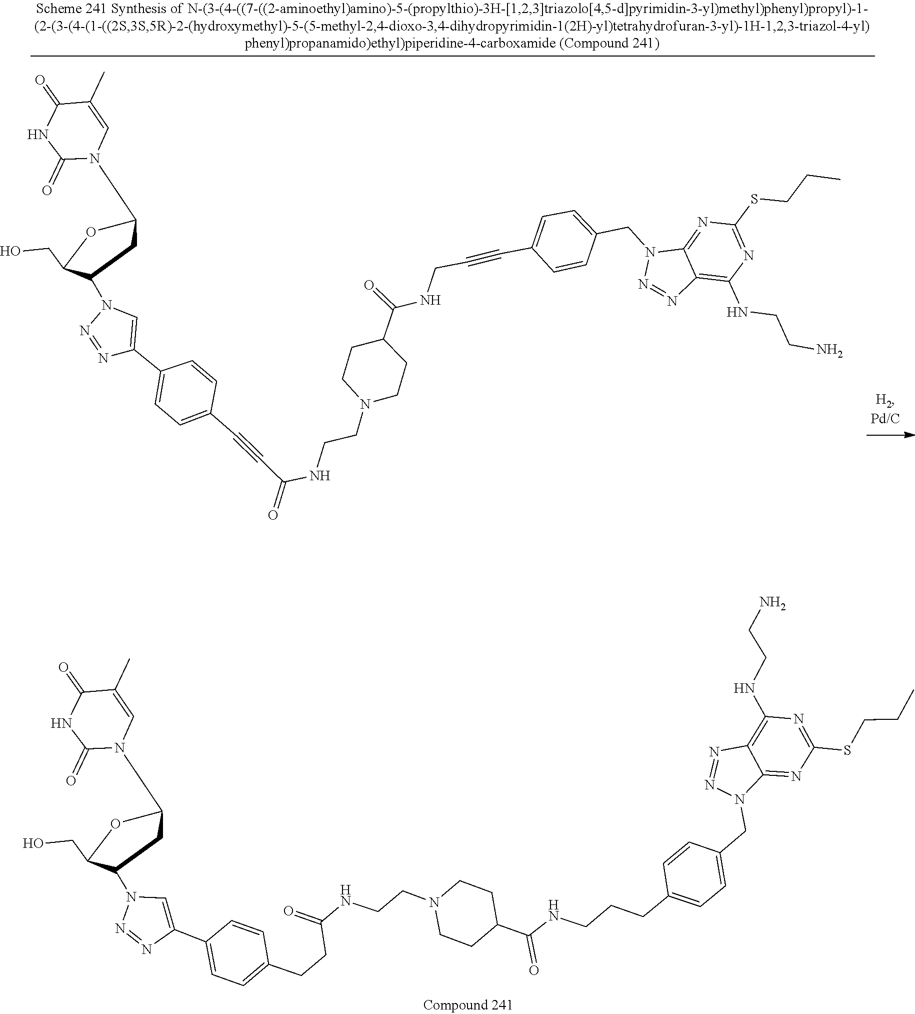 [SMILES: CCCSc1nc(NCCN)c2nnn(Cc3ccc(C#CCNC(=O)C4CCN(CCNC(=O)C#Cc5ccc(-c6cn([C@@H]7C[C@H](n8cc(C)c(=O)[nH]c8=O)O[C@@H]7CO)nn6)cc5)CC4)cc3)c2n1 ; CCCSc1nc(NCCN)c2nnn(Cc3ccc(CCCNC(=O)C4CCN(CCNC(=O)CCc5ccc(-c6cn([C@@H]7C[C@H](n8cc(C)c(=O)[nH]c8=O)O[C@@H]7CO)nn6)cc5)CC4)cc3)c2n1]

All publications and patent applications cited in this specification are herein incorporated by reference as if each individual publication or patent application were specifically and individually indicated to be incorporated by referenced. All crystal structures cited by RCSB PDB code are also incorporated by reference.

Although the foregoing invention has been described in some detail by way of illustration and example for the purposes of clarity of understanding, it will be readily apparent to one of ordinary skill in the art in light of the teaching of this invention that certain changes and modifications may be made thereto without departing from the spirit or scope of the invention as defined in the embodiments and/or claims.

1-279. (canceled)

280. A compound of Formula 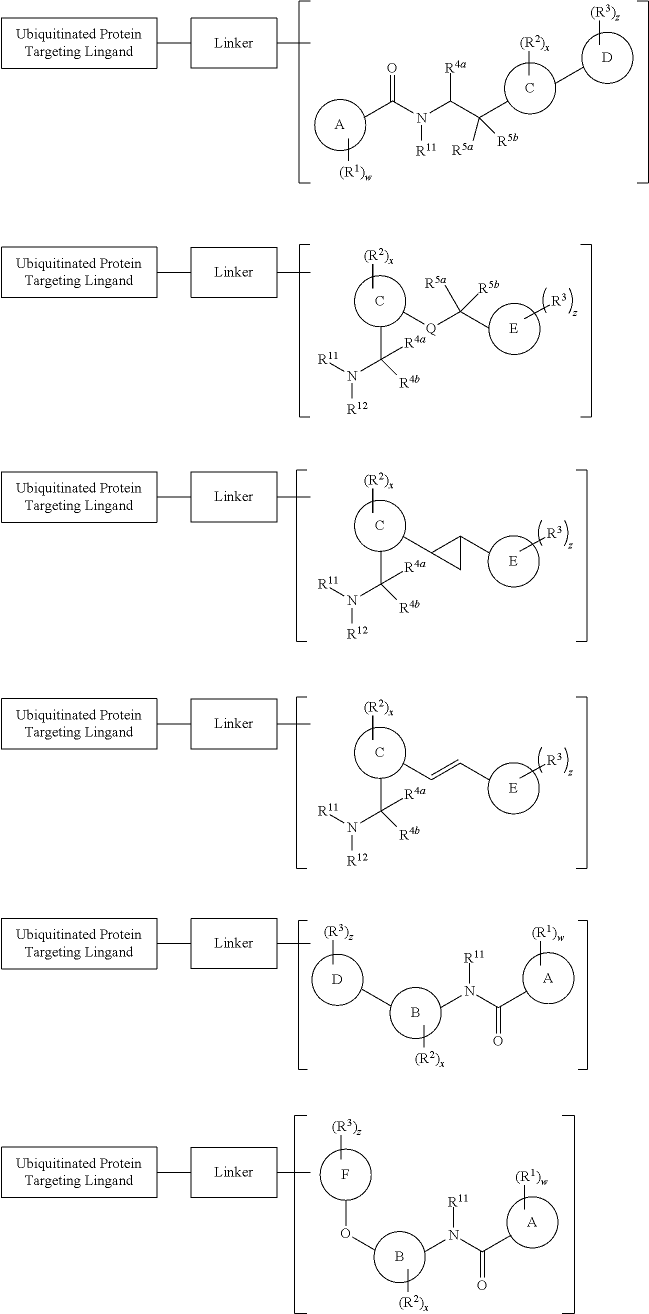 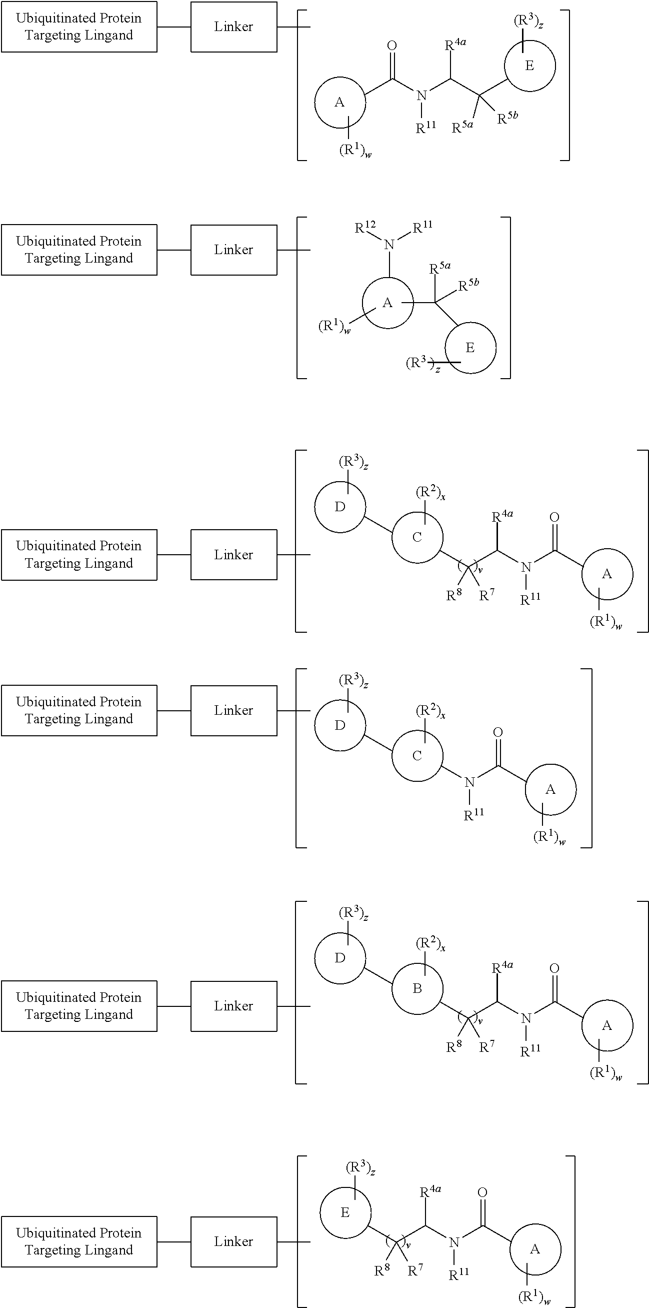
or a pharmaceutically acceptable salt thereof;
wherein: 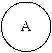
is aryl, heteroaryl, or bicycle; 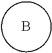
is a bicycle; 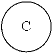
is aryl, heteroaryl, or bicycle; 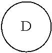
is a heterocycle; 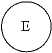
is aryl or heteroaryl, and 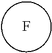
is a heterocycle bonded through a carbon atom;
wherein:
v is 0, 1, 2, or 3;
w is 0, 1, 2, 3, or 4 as allowed by valence;
x is 0, 1, 2, 3, or 4 as allowed by valence;
z is 0, 1, 2, 3, or 4 as allowed by valence;
Q is O, NR¹¹, CR⁷R⁸, or S;
R¹ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²¹;
R² is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —OC(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²²;
R³ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²³;
R^{4a} and R^{5a} are independently selected at each instance from hydrogen, halogen, alkyl, haloalkyl, alkenyl, alkynyl, heterocycle, aryl, heteroaryl, wherein each alkyl, haloalkyl, alkenyl, alkynyl, heterocycle, aryl, and heteroaryl is optionally substituted as allowed by valence with 1, 2, 3, or 4 substituents selected from R²⁴; or
R^{4b} and R^{5b} are independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²⁵;
R^{4a} and R^{4b} together with the atom to which they are attached are combined to form a spirocycle; or
R^{5a} and R^{5b} together with the atom to which they are attached are combined to form a spirocycle;
each R⁷ and R⁸ is independently selected from hydrogen, alkyl, and haloalkyl;
R¹⁰, is independently selected at each instance from hydrogen, alkyl, haloalkyl, alkenyl, alkynyl, —OR¹¹, —NR¹¹R¹², .SR¹¹, aryl, heterocycle, and heteroaryl; each of which alkyl, haloalkyl, alkenyl, alkynyl, heterocycle, aryl, and heteroaryl is optionally substituted as allowed by valence with 1, 2, 3, or 4 substituents selected from R³⁰;
R¹¹ and R¹² are independently selected at each instance from hydrogen, alkyl, haloalkyl, alkenyl, alkynyl, aryl, heterocycle, heteroaryl, —C(O)R⁴⁰, —S(O)R⁴⁰, and —S(O)₂R⁴⁰; each of which alkyl, haloalkyl, alkenyl, alkynyl, aryl, heterocycle, and heteroaryl is optionally substituted as allowed by valence with 1, 2, 3, or 4 substituents selected from R³¹;
R²¹, R²², R²³, R²⁴, and R²⁵ are independently selected at each instance from hydrogen, halogen, alkyl, haloalkyl, alkenyl, alkynyl, heterocycle, aryl, heteroaryl, cyano, nitro, —C(O)R⁴⁰, —OC(O)R⁴⁰, —NR⁴¹C(O)R⁴⁰, —OR⁴¹, —NR⁴¹R⁴², —S(O)R⁴⁰, —S(O)₂R⁴⁰, —OS(O)R⁴⁰, —OS(O)₂R⁴⁰, —NR⁴¹S(O)R⁴⁰, —NR⁴¹S(O)₂R⁴⁰, and —SR⁴¹, wherein each alkyl, haloalkyl, alkenyl, alkynyl, heterocycle, aryl, and heteroaryl is optionally substituted as allowed by valence with 1, 2, 3, or 4 substituents selected from R⁴³;
R³⁰ and R³¹ are independently selected at each instance from hydrogen, halogen, alkyl, haloalkyl, alkenyl, alkynyl, heterocycle, aryl, heteroaryl, cyano, nitro, —C(O)R⁴⁰, —OC(O)R⁴⁰, —NR⁴¹C(O)R⁴⁰, —OR⁴¹, —NR⁴¹R⁴², —S(O)R⁴⁰, —S(O)₂R⁴⁰, —OS(O)R⁴⁰, —OS(O)₂R⁴⁰, —NR⁴¹S(O)R⁴⁰, —NR⁴¹S(O)₂R⁴⁰, and —SR⁴¹, wherein each alkyl, haloalkyl, alkenyl, alkynyl, heterocycle, aryl, and heteroaryl is optionally substituted as allowed by valence with 1, 2, 3, or 4 substituents selected from R⁴³;
R⁴⁰ is independently selected at each instance from hydrogen, alkyl, haloalkyl, alkenyl, alkynyl, aryl, heterocycle, heteroaryl, amino, hydroxyl, alkoxy, —NHalkyl, and —N(alkyl)₂, each of which except hydrogen is optionally substituted as allowed by valence with 1, 2, 3, or 4 substituents selected from R⁴³;
R⁴¹ and R⁴² are independently selected at each instance from hydrogen, alkyl, haloalkyl, alkenyl, alkynyl, aryl, heterocycle, and heteroaryl; each of which except hydrogen is optionally substituted as allowed by valence with 1, 2, 3, or 4 substituents selected from R⁴³;
R⁴³ is independently selected at each instance from hydrogen, halogen, cyano, nitro, alkyl, haloalkyl, alkenyl, alkynyl, aryl, heterocycle, heteroaryl, amino, hydroxyl, alkoxy, —NHalkyl, —N(alkyl)₂, —OC(O)alkyl, —NHC(O)alkyl, and —N(alkyl)C(O)alkyl;
Ubiquitinated Protein Targeting Ligand is a ligand that binds a Target Ubiquitinated Protein; and
wherein Linker is of Formula: 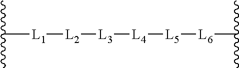
wherein
L₁, L₂, L₃, L₄, L₅, and L₆ are independently selected from the group consisting of a bond, alkyl, alkene, alkyne, haloalkyl, alkoxy, aryl, heterocycle, heteroaryl, bicycle, —C(O)—, —C(O)O—, —OC(O)—, —SO₂—, —S(O)—, —C(S)—, —C(O)NR¹¹, NR¹¹C(O)—, —O—, —S—, —P(O)(OR¹¹)O—, —P(O)(OR¹¹)—, polyethylene glycol, lactic acid, and glycolic acid, each of which except bond is optionally substituted with 1, 2, 3, or 4 substituents independently selected from R⁴⁴; wherein L₁, L₂, L₃, L₄, L₅, and L₆ are selected such that there are no more than two of the same moieties connected together (e.g, L₁, L₂, and L₃ cannot all three be —C(O)—) and O and N atoms are not directly linked together except within aromatic rings (e.g. L₁ and L₂ cannot both be —O—or NR¹¹);
R⁴⁴ is independently selected at each instance from hydrogen, halogen, alkyl, haloalkyl, alkenyl, alkynyl, aryl, heterocycle, heteroaryl, amino, hydroxyl, alkoxy, —NR¹¹R¹², halogen, cyano, nitro, —OC(O)R⁴⁰, —NR¹¹C(O)R⁴⁰—C(O)R⁴⁰, —OP(O)(R⁴⁰)₂, —P(O)(R⁴⁰)₂, —NR¹¹P(O)(R⁴⁰)₂—SR¹¹, —OR¹¹. —S(O)R⁴⁰, —S(O)₂R⁴⁰, and —N(alkyl)C(O)R⁴⁰, each of which except hydrogen is optionally substituted as allowed by valence with 1, 2, 3, or 4 substituents selected from R⁴⁵;
R⁴⁵ is independently selected at each instance from hydrogen, halogen, cyano, nitro, alkyl, haloalkyl, alkenyl, alkynyl, aryl, heterocycle, heteroaryl, amino, hydroxyl, alkoxy, —NHalkyl, and —N(alkyl)₂, —OC(O)alkyl, —NHC(O)alkyl, and —N(alkyl)C(O)alkyl; and
Linker replaces or is covalently attached to a R¹, R², R³, R^{4a}, R^{4b}, R^{5a}, R^{5b}, R⁷, R⁸, R¹⁰, R¹¹, or R¹².

281. The compound of claim 280, wherein Linker-Ubiquitinated Protein Targeting Ligand replaces a R¹, R², R³, RR^{4b}, R^{5a}, R^{5b}, R⁷, R⁸, R¹⁰, R¹¹, or R¹² group.

282. The compound of claim 280, wherein Linker-Ubiquitinated Protein Targeting Ligand is covalently attached to a R¹, R², R³, R^{4a}, R^{4b}, R^{5a}, R^{5b}, R⁷, R⁸, R¹⁰, R¹¹, or R¹² group as allowed by valence.

283. The compound of claim 280, wherein the compound is of any one of the following Formulae: 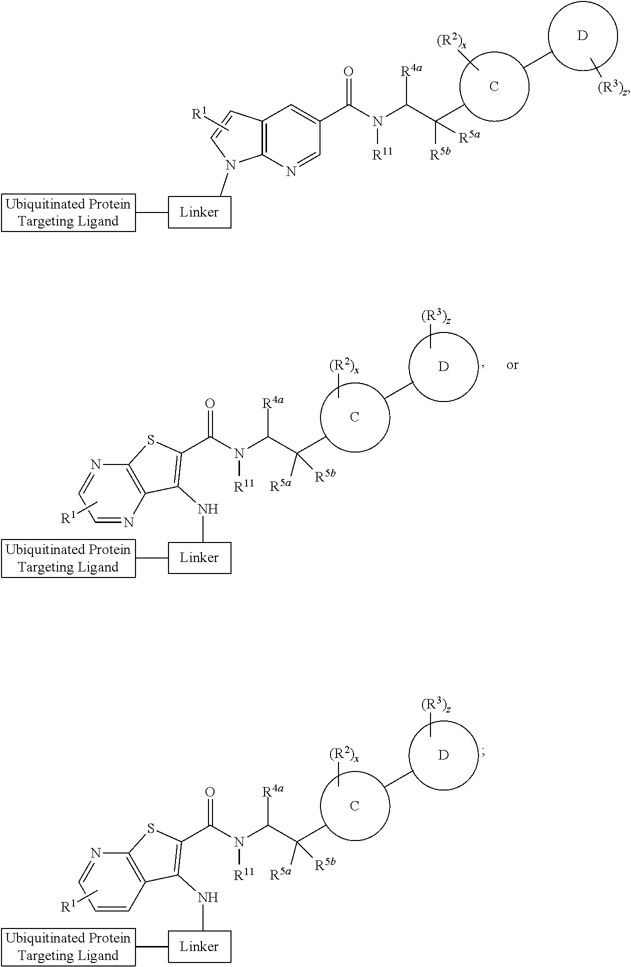
or a pharmaceutically acceptable salt thereof.

284. The compound of claim 280, wherein the compound is of any one of the following Formulae: 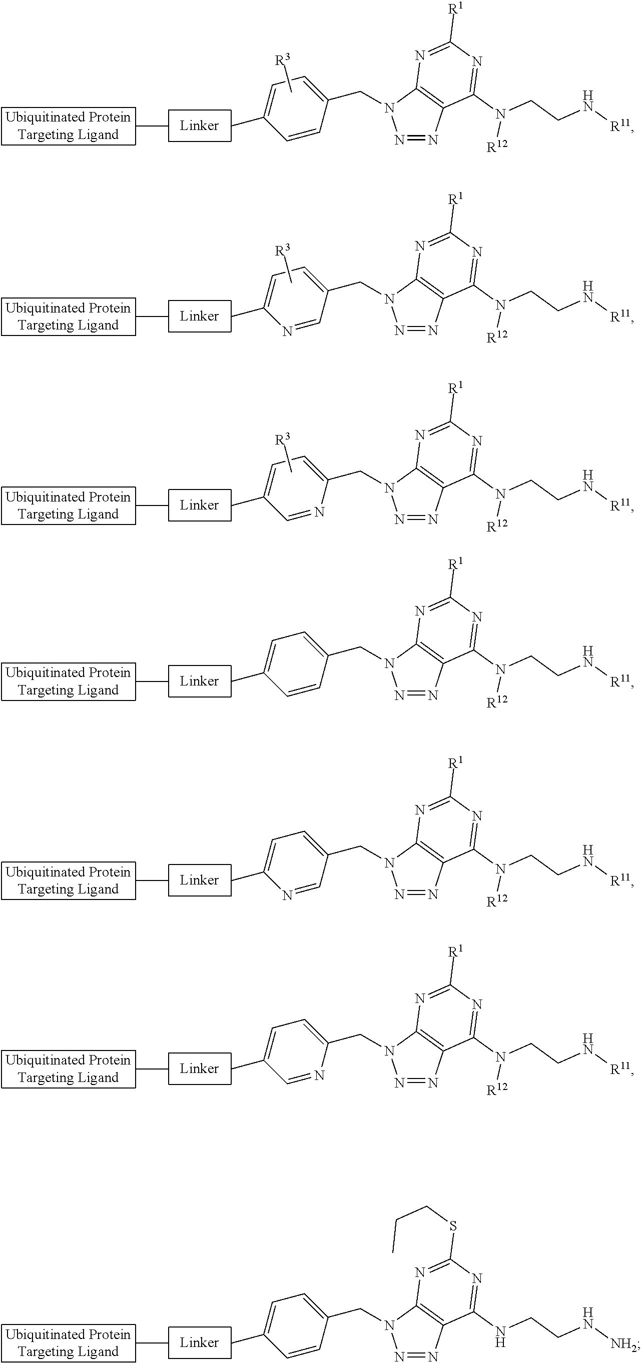
or a pharmaceutically acceptable salt thereof.

285. The compound of claim 280, wherein R¹ is hydrogen, halogen, cyano, nitro, —C(O)R¹⁰, —OC(O)R¹⁰, —NR¹¹C(O)R¹⁰, OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, or —SR¹¹.

286. The compound of claim 280, wherein R¹ is alkyl, haloalkyl, alkenyl, alkynyl, heterocycle, aryl, or heteroaryl, wherein the alkyl, haloalkyl, alkenyl, alkynyl, heterocycle, aryl, or heteroaryl is optionally substituted as allowed by valence with 1, 2, 3, or 4 substituents selected from R²¹.

287. The compound of claim 280, wherein the compound is of any one of the following Formulae: 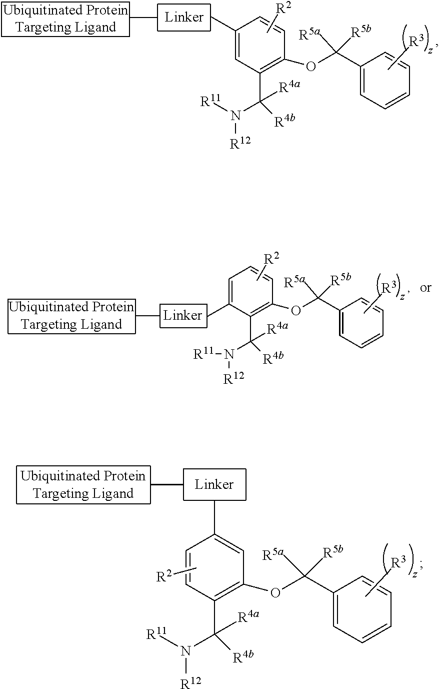
or a pharmaceutically acceptable salt thereof.

288. The compound of claim 280, wherein R² is hydrogen, halogen, cyano, nitro, —C(O)R¹⁰, —OC(O)R¹⁰, —OC(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, or —SR¹¹.

289. The compound of claim 280, wherein R² is alkyl, haloalkyl, alkenyl, alkynyl, heterocycle, aryl, or heteroaryl, wherein the alkyl, haloalkyl, alkenyl, alkynyl, heterocycle, aryl, or heteroaryl is optionally substituted as allowed by valence with 1, 2, 3, or 4 substituents selected from R²².

290. The compound of claim 280, wherein R³ is hydrogen.

291. The compound of claim 280, wherein R³ is halogen.

292. The compound of claim 280, wherein R³ alkyl optionally substituted with 1, 2, 3, or 4 substituents selected from R²³.

293. The compound of claim 280, wherein the Ubiquitinated Protein Targeting Ligand is a ligand that binds CFTR, phenylalanine hydroxylase, p53, rhodopsin, c-myc., RIPK1, CDKN1B, ABCA4, ABCB11, choline acetylase, CYLD, NEMO, AH receptor-interacting protein, PCDC4, RIPK2, BAX, P21, SERPINA1, PKLR, KEAP1, PTEN, IRAK4, TK2, KCNQ1, or STING1.

294. The compound of claim 280, wherein the compound is selected from any one of the compounds depicted in Table 1, or a pharmaceutically acceptable salt thereof.

295. A pharmaceutical composition comprising an effective amount of a compound of claim 280 or a pharmaceutically acceptable salt thereof and a pharmaceutically acceptable carrier.

296. A method of treating a disorder mediated by the Target Ubiquitinated Protein in a human comprising administering an effective amount of a compound of claim 280, or a pharmaceutically acceptable salt thereof.
